SEQUENCE LISTING (1 sequence)

SEQ ID NO: 1 - protein, 727 aa, Thermus thermophilus
MVGADLGLWNRLEPALAYLAPEERAKVREAYRFAEEAHRGQLRRSGEPYITHPVAVAEILAGLQMDADTVAAGLLHDTVEDCGVAPEELERRFGPTVRRIVEGETKVSKLYKLANLEGEERRAEDLRQMFIAMAEDVRIIIVKLADRLHNLRTLEHMPPEKQKRIAQETLEIYAPLAHRLGMGQLKWELEDLSFRYLHPEAFASLSARIQATQEARERLIQKAIHLLQETLARDELLQSQLQGFEVTGRPKHLYSIWKKMEREGKTLEQIYDLLAVRVILDPKPAPTRESQALREKQVCYHVLGLVHALWQPIPGRVKDYIAVPKPNGYQSLHTTVIALEGLPLEVQIRTREMHRVAEYGIAAHWLYKEGLTDPEEVKRRVSWLKSIQEWQKEFSSSREFVEAVTKDLLGGRVFVFTPKGRIINLPKGATPVDFAYHIHTEVGHHMVGAKVNGRIVPLSYELQNGEIVEILTSKNAHPSKGWLEFAKTRSAKSKIRQYFRAQERQETLERGQHLLERYLKRRGLPKPTDSQLEEAAKRLSLPPSPEELYLALALNRLTPRQVAEKLYPKALLKPEKPKPQVRNEWGIRLEQDLQAPIRLASCCEPMKGDPILGFVTRGRGVTVHRADCPNLRRLLQGPEADRVIGAYWEGVGGKVVVLEVLAQDRAGLLRDVMQVVAEAGKSALGSETRVLGPLARIRLRLAVQDGEEERILQAVQKVSGVKEARWA

(19) United States
(12) Patent Application Publication
(10) Pub. No.: US 2023/0128889 A1
(43) Pub. Date: Apr. 27, 2023

(54) REL/RELA/SPOT SMALL MOLECULES MODULATORS AND SCREENING METHODS

(71) Applicants: Université Libre de Bruxelles, Bruxelles (BE); Universitat Autónoma de Barcelona, Bellaterra (ES)

(72) Inventors: Abel GARCIA PINO, Bruxelles (BE); Cédric Pierre GOVAERTS, Uccle (BE); Hanna AINELO, Etterbeek (BE); Hedvig TAMMAN, Uccle (BE); Vasili HAURYLIUK, Umeå (SE); Leonardo PARDO CARASCO, Barcelona (ES); Minos Timotheos MATSOUKAS, Athens (GR)

(73) Assignees: Université Libre de Bruxelles, Bruxelles (BE); Universitat Autónoma de Barcelona, Bellaterra (ES)

(21) Appl. No.: 17/915,155

(22) PCT Filed: Mar. 30, 2020
(86) PCT No.: PCT/EP2020/059005

§ 371 Date: Sep. 28, 2022

(51) Int. Cl. G16C 20/64; G16C 20/50; G16B 15/30

(52) U.S. Cl. CPC G16C 20/64; G16C 20/50; G16B 15/30

(57) ABSTRACT

The present invention concerns screening methods to identify compounds that regulate activity of RSH enzymes such as Rel, and specifically Rel synthetase and/or Rel hydrolase activity. Also intended are compounds that interact and regulate Rel synthetase and/or hydrolase activity. These compounds are valuable to target persister cells not affected by traditional antibiotics.

### FIELD OF THE INVENTION

The invention relates to the elucidation of the different forms of the Rel enzyme crystal structure, to screening methods to identify Rel modulators binding into the catalytic site of said crystal structures and to the molecules identified thereby. The invention is of particular interest to the field of molecular biology, more particular in the development of anti-persister drugs against microorganisms such as antibiotic resistant bacteria.

### BACKGROUND OF THE INVENTION

The overuse and misuse of antibiotics combined with a lack of progress in the development of new antibacterial drugs have led to the emergence of pathogenic antibiotic resistant bacteria. The incidence of these bacteria (also known as “superbugs”) is increasing at an alarming rate, and thus bacterial infections are resurging as a prominent threat to human health (Ventola, The antibiotic resistance crisis, Pharmacy and therapeutics, 2015). In the last years, multiple health instances have repeatedly warned about these pathogenic antibiotic (multi)resistant bacteria and the threats they pose to human health (Michael et al., The antimicrobial resistance crisis: causes, consequences, and management, Frontiers in public health, 2013).

One mechanism that bacteria use to survive in presence of antibiotics is by the phenomenon of bacterial persistence. Whereas the majority of a bacterial population will proliferate quickly in an infected host organism, a smaller fraction of this population will actively suppress growth. Since the majority of all clinically used antibiotics target rapidly dividing bacteria, the small population of bacteria in the persistence state will not be affected by these drugs and are able to switch back to their normal, non-persistent state post-antibiotic treatment(s). Hence, persister cells are a primary source of chronic infections because they are difficult or often even impossible to eradicate using conventional antibiotics. Additionally, these persisters provide a viable cell reservoir wherefrom resistant mutants can arise, because mutations increasing antibiotic tolerance favor selection of resistance mutations (Windels et al., Bacterial persistence promotes the evolution of antibiotic resistance, 2019).

A growing body of experimental evidence supports the notion that the stringent response, a bacterial phenotypic resetting that is crucial to cope with adverse environmental changes, is intricately involved in the formation of persister cells. A crucial mediator of this stringent response is the alarmone guanosine polyphosphate (guanosine 3′,5′-bisdiphosphate and guanosine 5′-triphosphate-3′-diphosphate), abbreviated as (p)ppGpp. The levels of (p)ppGpp are tightly regulated by the concerted opposing activities of RelA/SpoT homologue (RSH) enzymes that can both transfer a pyrophosphate group of ATP to the 3′ position of GDP (or GTP) or remove the 3′ pyrophosphate moiety from (p)ppGpp (Geiger et al., Role of the (p)ppGpp Synthase RSH, a RelA/SpoT Homolog, in Stringent Response and Virulence of Staphylococcus aureus, Infection and immunity, 2010). While RSH enzymes are universally conserved in bacteria, they are not present in humans which mark them as a very promising drug target. While progress is being made, these bifunctional RSH enzymes have proven to be difficult to structurally characterize since they display poor stability upon crystallization and have a tendency to aggregate. Despite a considerable amount of research, it remains unclear how the two opposing activities of Rel are controlled at the molecular level.

Taken together, there is an unmet need to develop innovative strategies that are effective in counteracting pathogenic antibiotic resistant bacteria and bacterial persistence in general, including new classes of antimicrobials, vaccines, and treatment strategies. Approaches which allow screening for compounds that are able to modulate one or both activities of RSH enzymes entail great value to generate these novel antimicrobials.

### SUMMARY OF THE INVENTION

The present inventors have identified the structural changes that Rel, an RSH enzyme, undergoes upon ligand binding and which are necessary to perform its biological functions (i.e. Rel hydrolase and synthetase activity). Furthermore, the inventors have developed new screening methods to identify compounds that interfere with these conformational changes. Finally, promising Rel interacting compounds have been identified by these methods and are a potent manner of steering Rel activity, and thus counteracting persister cells.

As evidenced in the Examples section, by elucidation of the three dimensional structure of the complete Rel enzyme at an unprecedented resolution, distinct conformations of the protein could be observed which are each connected to different activity states of Rel. This information evidences the presence of an allosteric mechanism which acts as a Rel activity switch. The binding of GDP/ATP stretches apart the N-terminal catalytic domains of Rel_{Tf} (Rel_{Tt}^{NTD}) activating the synthetase domain and allosterically blocking the hydrolase active site. Conversely, binding of ppGpp unlocks the hydrolase domain and triggers recoil of both NTDs, which partially buries the synthetase active site and precludes the binding of synthesis precursors. By further extensive structural analyses, key atomic coordinates of the three dimensional structures of Rel were identified that are able to discriminate the different conformational states. In addition, key amino acid residues were discovered in the protein structure that are of high value for candidate compounds to interact with and to modulate Rel synthetase and/or hydrolase activity. Based on this newly obtained structural information, screening methods were developed to identify such compounds. By using this novel screening approach, the inventors have found Rel interacting compounds that are capable of steering Rel activity, and are thus ultimately capable of counteracting persister cells.

The invention therefore relates to the following aspects:
1. A method for identifying compounds that modulate Rel hydrolase and/or Rel synthetase activity comprising the step of employing a three dimensional structure represented by a set of atomic coordinates presented in Table 1, 2, 3, or 4 or a subset thereof, or atomic coordinates which deviate from those in Table 1, 2, 3, or 4, or a subset thereof, by a root mean square deviation (RMSD) of residue over protein backbone atoms by no more than 3 Å and assessing the degree of fit of a candidate compound to said three-dimensional protein structure of Rel.
2. The method according to aspect 1, whereby interactions of said candidate compound with one or more amino acid residues of a region on the surface of the protein defined by amino acid residues: Arg43, Ser45, His156, Thr153, Met157, Asn150, Leu154, Lys161, Arg147, Lys143, Glu168, and Ile165 of the Rel amino acid sequence as defined in SEQ ID NO: 1 or an amino acid sequence with at least 70% sequence identity to the amino acid sequence of SEQ ID NO: 1 indicate the candidate compound is a modulator of Rel hydrolase activity, or of Rel hydrolase and synthetase activity.
3. The method according to aspect 1, whereby interactions of said candidate compound with one or more amino acid residues of a region on the surface of the protein defined by amino acid residues: Asn327, Tyr329, Lys325, His333, Arg277, Arg349, Gln347, Glu345, Asp272, Arg316, Lys251, Arg249, Ala275, Arg355, Ser255, and Lys186 of the Rel amino acid sequence as defined in SEQ ID NO: 1 or an amino acid sequence with at least 70% sequence identity to the amino acid sequence of SEQ ID NO: 1 indicate the candidate compound is a modulator of Rel synthetase activity or of Rel synthetase and hydrolase activity.
4. The method according to aspect 1, whereby interactions of said candidate compound with one or more amino acid residues of a region on the surface of the protein defined by amino acid residues: Lys164, Asp200, Tyr201, Arg204, Tyr211, Lys212, His219, Arg221, Arg222, Arg225 of the Rel amino acid sequence as defined in SEQ ID NO: 1 or an amino acid sequence with at least 70% sequence identity to the amino acid sequence of SEQ ID NO: 1 indicate the candidate compound is an allosteric compound or an effector of the Rel synthetase and/or hydrolase activity.
5. The method according to any one of aspects 1 to 4, further comprising determining a score of said candidate compound to modulate Rel hydrolase and/or Rel synthetase activity based on the number of interactions with said amino acid residues.
6. The method according to anyone of aspects 1 to 5, further comprising comparing the conformational state of Rel before and after said candidate compound binds to Rel, wherein a change in conformational state is indicative for the candidate compound to be a bona fide modulator of Rel hydrolase and/or Rel synthetase activity, preferably wherein the conformational state of Rel before candidate compound binding is the resting conformational state characterized by the atomic coordinates of Table 1.
7. The method according to any one of aspects 1 or 2, further comprising comparing the conformational state of Rel with or without said candidate compound binding to Rel, wherein a change in conformational state is indicative for the candidate compound to be a bona fide modulator of Rel hydrolase or Rel hydrolase and synthetase activity, preferably wherein the conformational state of Rel without candidate compound binding is the (P)ppGpp bound conformational state characterized by the atomic coordinates of Table 3.
8. The method according to anyone of aspects 1 or 3, further comprising comparing the conformational state of Rel with or without said candidate compound binding to Rel, wherein a change in conformational state is indicative for the candidate compound to be a bona fide modulator of Rel synthetase or Rel synthetase and hydrolase activity, preferably wherein the conformational state of Rel without candidate compound binding is the AMP-G4P bound conformational state characterized by the atomic coordinates of Table 2.
9. The method according to anyone of aspects 1 or 4, further comprising comparing the conformational state of Rel with or without said candidate compound binding to the allosteric site of Rel, wherein a change in conformational state is indicative for the candidate compound to be a bona fide effector of the Rel hydrolase and/or synthetase activity, preferably wherein the conformational state of Rel without candidate compound binding is the conformational state characterized by the atomic coordinates of Table 4.
10. The method according to aspect 7, wherein the candidate compound is considered a Rel hydrolase inhibitor when upon binding with one or more of said Rel amino acid residues, Rel is stabilized in an open state.
11. The method according to aspect 8, wherein the candidate compound is considered a Rel synthetase inhibitor when upon binding with one or more of said Rel amino acid residues, Rel is stabilized in a closed state.
12. The method according to any one of aspects 1 to 11, further comprising testing of the ability of the candidate compounds for modulating Rel synthetase and/or Rel hydrolase activity.
13. The method according to any of aspects 1 to 12, which is a computer-implemented method, said computer comprising an inputting device, a processor, an user interface, and an outputting device, wherein said method comprises the steps of:
   a) generating a three-dimensional structure of said atomic coordinates, or said subset thereof;
   b) fitting the structure of step a) with the structure of a candidate compound by computational modeling;
   c) selecting a candidate compound that possesses energetically favorable interactions with the structure of step a).
14. The method according to aspect 13, wherein said fitting comprises superimposing the structure of step a) with the structure of said candidate compound.
15. The method according to aspect 13 or 14, wherein said modeling comprises docking modeling.
16. The method according to any one of aspects 13 to 15, wherein said candidate compound of step c) can bind to at least 1 amino acid residue of the structure of step a) without steric interference.
17. An in vitro method for identifying a compound which modulates Rel hydrolase and/or synthetase activity, comprising the steps of:
   a) providing a candidate compound;
   b) providing a Rel polypeptide;
   c) contacting said candidate compound with said Rel polypeptide;
   d) determining the hydrolase and/or synthetase activity of Rel in the presence and absence of said candidate compound; and
   e) identifying said candidate compound as a compound which modulates Rel hydrolase and/or synthetase activity if a change in activity is detected.
18. The method according to aspect 17, wherein said compound is inhibiting the hydrolase and/or synthetase activity of Rel; or wherein said compound is stimulating the hydrolase and/or synthetase activity of Rel.
19. The method according to aspect 17 or 18, wherein said Rel polypeptide is as defined in SEQ ID NO:1 or has at least 70% sequence identity to the amino acid sequence of as defined in SEQ ID NO: 1.
20. A modulator of Rel hydrolase and/or synthetase activity obtained by the methods of any one of aspects 1 to 19.
21. The modulator according to aspect 20, which is an inhibitor of the Rel hydrolase and/or synthetase activity.
22. The modulator according to aspect 20, which is an effector of the Rel hydrolase and/or synthetase activity, or which is a compound increasing the Rel hydrolase and/or synthetase activity.
23. The modulator according to any one of aspects 20 to 22,
   wherein said modulator is a compound of formula (I), or an isomer, preferably a stereo-isomer or a tautomer, a solvate, a salt, preferably a pharmaceutically acceptable salt, or a prodrug thereof,

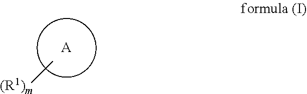

wherein m is an integer selected from 1, 2, 3, 4, 5, 6, or 7;
wherein each R¹ is independently selected from halogen, ═O, nitro, or a group comprising hydroxyl, —SH, —NH₂, C(O)OH, heterocyclyl, heteroaryl, alkyl, haloalkyl, cycloalkyl, cycloalkenyl, hydroxycarbonylalkyl, hydroxycarbonylalkenyl, alkenyl, aryl, heteroalkyl, heteroalkenyl, alkyloxy, arylalkyl, arylalkenyl-, aryl-heteroalkyl-, aryl-heteroalkenyl-, aryl-heteroaryl-; aryl-heterocyclyl-, heterocyclyl-alkyl-, heterocyclyl-alkenyl-, heterocyclyl-heteroalkyl-, heterocyclyl-heteroalkenyl-, heteroaryl-alkyl-, heteroaryl-alkenyl-, heteroaryl-heteroalkyl-, heteroaryl-heteroalkenyl-, alkyloxy-, haloalkoxy-, alkenyloxy-, aryloxy-; heteroaryloxy-, heterocylyloxy-, alkylthio-, alkenylthio-, arylthio-, heteroarylthio-, heterocyclylthio-, aryl-alkyloxy-, heteroaryl-alkyloxy-, heterocyclyl-alkyloxy-, aryl-alkylthio-, heteroaryl-alkylthio-, heterocyclyl-alkylthio-, alkyl-SO2-, alkenyl-SO2-, heteroalkyl-SO2-, heteroalkenyl-SO2-, aryl-SO2-, heteroaryl-SO2-, heterocyclyl-SO2-, Heteroaryl-heteroalkyl-heteroaryl-, Heteroaryl-heteroalkenyl-heteroaryl-, heteroaryl-alkyl-heteroaryl-, Heteroaryl-alkenyl-heteroaryl-, Aryl-heteroaryl-alkyl-, aryl-heteroaryl-heteroalkyl-, aryl-heteroaryl-alkenyl-, aryl-heteroaryl-heteroalkenyl-, Alkyloxy-aryl-alkyl-, Alkyloxy-heteroaryl-alkyl-, Alkyloxy-aryl-alkenyl-, Alkyloxy-heteroaryl-alkenyl-, Alkyloxy-heterocyclyl-alkyl-, Alkyloxy-heterocyclyl-alkenyl-, Alkyloxy-heterocyclyl-heteroalkyl-, Alkyloxy-heterocyclyl-heteroalkenyl-, aryl-alkenyl-heteroaryl-heteroalkyl, aryl-alkyl-heterocyclyl-heteroalkyl, Aryl-heteroalkyl-heteroaryl-, aryl-heteroalkenyl-heteroaryl-, aryl-heteroalkyl-heterocyclyl-, aryl-heteroaryl-heterocyclyl-; Aryl-heteroalkyl-heteroaryl-alkyl-, alkenyl-aryl-heteroalkenyl, Aryl-alkyl-heterocyclyl-SO2-, aryl-alkenyl-heterocyclyl-SO2-, heteroaryl-alkyl-heterocyclyl-SO2-, heteroaryl-alkenyl-heterocyclyl-SO2-, Aryl-heteroalkenyl-heteroaryl-alkyl-, alkenyl-aryl-heteroalkenyl-, Aryl-Alkyl-heterocyclyl-alkyl-, Aryl-Alkyl-heterocyclyl-alkenyl-, Aryl-Alkyl-heterocyclyl-heteroalkyl-, Aryl-Alkyl-heterocyclyl-heteroalkenyl-, Aryl-alkenyl-heterocyclyl-alkyloxy-, Aryl-alkyl-heterocyclyl-alkyloxy-, Aryl-alkenyl-heteroaryl-alkyloxy-, Aryl-alkyl-heteroaryl-alkyloxy-, aryl-imino-, heteroalkyl-aryl-imino-, alkenyl-aryl-imino-; and wherein each of said groups can be unsubstituted or substituted with one or more substituents each independently selected from the group comprising halogen, nitro, oxo, alkyloxy, —C(O)OH, —NH₂, hydroxycarbonylalkyl, hydroxycarbonylalkenyl, hydroxyl, alkyl, alkenyl, haloalkyl, haloalkenyl, heteroalkyl, heteroalkenyl, ═S, —SH, aryl, nitroaryl-, heteroaryl, heterocyclyl; aryl-alkyl-; aryl-alkenyl-; arylheteroalkyl-; arylheteroalkenyl-; heterocyclyl-alkyl-imino, aryl-imino-, heteroalkyl-aryl-imino-; and
wherein cycle A is selected from the group represented by formula (Ia);

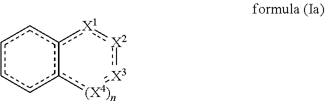

wherein the dotted line represents an optional double bond;
wherein n is an integer selected from 0 or 1;
wherein each of X¹, X², X³, and X⁴ is independently selected from the group comprising N, NH, S, O, C═O, C═S, CH, C(Z¹)₂, and N(Z²), and
   wherein each Z¹ is independently selected from selected from the group comprising hydrogen, halogen, nitro, hydroxyl, —SH, —NH₂, —C(O)OH, alkyl, alkenyl, hydroxycarbonylalkyl, hydroxycarbonylalkenyl, heteroalkyl, heteroalkenyl, aryl, heteroaryl, heterocyclyl, alkyloxy, alkylthio, arylalkyl-, aryl-alkenyl-, aryl-heteroalkyl-, aryl-heteroalkenyl-, heterocyclyl-heteroalkyl, heterocyclyl-heteroalkenyl-, heteroaryl-heteroalkyl-, heteroaryl-heteroalkenyl-, Heteroaryl-heteroalkyl-heteroaryl-, Heteroaryl-heteroalkenyl-heteroaryl-, Aryl-heteroaryl-alkyl-, Aryl-heteroaryl-heteroalkyl-, Aryl-heteroaryl-alkenyl, Aryl-heteroaryl-heteroalkenyl, Alkyloxy-aryl-alkyl-, and Alkyloxy-aryl-alkenyl-; and
   wherein each Z² is independently selected from the group comprising alkyl, alkenyl, hydroxycarbonylalkyl, hydroxycarbonylalkenyl, heteroalkyl, heteroalkenyl, aryl, heteroaryl, heterocyclyl, aryl-heteroalkyl-, aryl-heteroalkenyl-, heterocyclyl-heteroalkyl, heterocyclyl-heteroalkenyl-, heteroaryl-heteroalkyl-, heteroaryl-heteroalkenyl-, Heteroaryl-heteroalkyl-heteroaryl-, Heteroaryl-heteroalkenyl-heteroaryl-, Aryl-heteroaryl-alkyl-, Aryl-heteroaryl-heteroalkyl-, Aryl-heteroaryl-alkenyl, Aryl-heteroaryl-heteroalkenyl, Alkyloxy-aryl-alkyl-, and Alkyloxy-aryl-alkenyl-; or
wherein when X² and X³ are each independently selected from C(Z¹)₂ or N(Z²) as defined above, two Z¹, or Z¹ together with Z² together with the atom to which they are attached form a ring selected from the group comprising heterocyclyl, cycloalkyl, cycloalkenyl, aryl, and heteroaryl.
or
wherein said modulator is a compound of formula (II), or an isomer, preferably a stereo-isomer or a tautomer, a solvate, a salt, preferably a pharmaceutically acceptable salt, or a prodrug thereof,

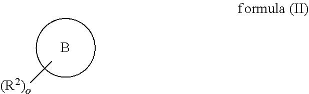

wherein o is an integer selected from 1, 2, 3, 4, 5, 6, or 7; and preferably selected from 1, 2, 3, 4, or 5, and preferably selected from 1, 2, 3, or 4;
wherein each R² is independently selected from halogen, ═S, ═O, nitro, or a group comprising hydroxyl, —SH, —NH₂, —C(O)OH, alkyl, alkenyl, haloalkyl, cycloalkyl, cycloalkenyl, heteroalkyl, heteroalkenyl, aryl, heteroaryl, heterocyclyl, arylalkyl-, arylalkenyl-, aryl-heteroalkyl-, aryl-heteroalkenyl-, aryl-heteroaryl-; aryl-heterocyclyl-, heterocyclyl-alkyl-; heterocyclyl-alkenyl-, heterocyclyl-heteroalkyl-, heterocyclyl-heteroalkenyl-, heteroaryl-alkyl-, heteroaryl-alkenyl-, heteroaryl-heteroalkyl-, heteroaryl-heteroalkenyl-, alkyloxy, haloalkoxy, alkenyloxy, aryloxy; heteroaryloxy, heterocylyloxy, alkylthio, alkenylthio, arylthio, heteroarylthio, heterocyclylthio, aryl-alkyloxy-, heteroaryl-alkyloxy-, heterocyclyl-alkyloxy-, aryl-alkylthio-, heteroaryl-alkylthio-, heterocyclyl-alkylthio-, hydroxycarbonylalkyl-, hydroxycarbonylalkenyl-, alkyl-SO2-, alkenyl-SO2-, heteroalkyl-SO2-, heteroalkenyl-SO2-, aryl-SO2-, heteroaryl-SO2-, heterocyclyl-SO2-, heteroaryl-alkyl-SO2-; heteroaryl-alkenyl-SO2-, heteroaryl-heteroalkyl-SO2-, heteroaryl-heteroalkenyl-SO2- and heteroaryl-NH—SO2-,
   and wherein each of said groups can be unsubstituted or substituted with one or more substituents each independently selected from the group comprising halogen, nitro, oxo (═O), alkyloxy, —C(O)OH, —NH₂, hydroxycarbonylalkyl, hydroxycarbonylalkenyl, hydroxyl, alkyl, alkenyl, heteroalkyl, heteroalkenyl ═S, —SH, aryl, heteroaryl, heterocyclyl.
wherein cycle B is selected from the group represented by formula (IIa);

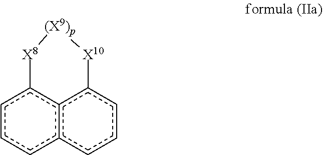

wherein the dotted line represents an optional double bond;
wherein p is an integer selected from 0 or 1;
wherein each of X⁸, X⁹, and X¹⁰ is independently selected from NH, N—OH, S, O, C═O, C═S, C(Z³)₂ and N(Z⁴), and wherein each Z³ is independently selected from the group comprising hydrogen, halogen, nitro, hydroxyl, —SH, —NH₂, —C(O)OH, alkyl, alkenyl, hydroxycarbonylalkyl, hydroxycarbonylalkenyl, heteroalkyl, heteroalkenyl, aryl, heteroaryl, heterocyclyl, and alkyloxy, and wherein each Z⁴ is independently selected from the group comprising alkyl, alkenyl, hydroxycarbonylalkyl, hydroxycarbonylalkenyl, heteroalkyl, heteroalkenyl, aryl, heteroaryl, heterocyclyl, and alkyloxy;
   and preferably with the proviso that when p is 1 and X⁹ is N—OH, then X⁸ and X¹⁰ are C═O, and/or
   preferably with the proviso that when p is 0 and X⁸ is NH, then X¹⁰ is C═O, or when p is 0 and X⁸ is C═O, then X¹⁰ is NH.
or
wherein said modulator is a compound of formula (III), or an isomer, preferably a stereo-isomer or a tautomer, a solvate, a salt, preferably a pharmaceutically acceptable salt, or a prodrug thereof,

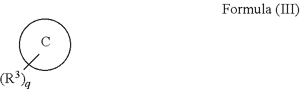

wherein q is an integer selected from 1, 2, 3, 4, 5, or 6; and
wherein each R³ is independently selected from halogen, ═S, ═O, nitro, or a group comprising hydroxyl, —SH, —NH₂, —C(O)OH, alkyl, alkenyl, haloalkyl, hydroxycarbonylalkyl, hydroxycarbonylalkenyl, cycloalkyl, cycloalkenyl, heteroalkyl, heteroalkenyl, aryl, heteroaryl, heterocyclyl, alkyloxy, alkylthio, heterocyclyl-alkyl-, heterocyclyl-heteroalkyl-, heterocyclyl-heteroaryl, aryl-alkyl-, arylalkenyl-, aryl-heteroalkyl-; aryl-heteroalkenyl-, aryl-heteroaryl-; aryl-heterocyclyl-, heterocyclyl-alkyl-; heterocyclyl-alkenyl-, heterocyclyl-heteroalkenyl-, heteroarylalkyl-, heteroarylalkenyl-, heteroaryl-heteroalkyl-, heteroaryl-heteroalkenyl-, alkyloxy-, alkenyloxy-, aryloxy-; heteroaryloxy-, heterocylyloxy-, alkylthio-, alkenylthio-, arylthio-, heteroarylthio-, heterocyclylthio-, aryl-alkyloxy-, heteroaryl-alkyloxy-, heterocyclyl-alkyloxy-, aryl-alkylthio-, heteroaryl-alkylthio-, heterocyclyl-alkylthio-, alkyl-SO2-, alkenyl-SO2-, heteroalkyl-SO2-, heteroalkenyl-SO2-, aryl-SO2-, heteroaryl-SO2-, heterocyclyl-SO2-, heteroaryl-heteroalkyl-heteroaryl-, Heteroaryl-heteroalkenyl-heteroaryl-, heteroaryl-alkyl-heteroaryl-, Heteroaryl-alkenyl-heteroaryl-, Aryl-heteroaryl-alkyl-, aryl-heteroaryl-alkenyl-, aryl-heteroaryl-heteroalkyl-, aryl-heteroaryl-heteroalkenyl-, aryl-alkenyl-, Alkyloxy-aryl-alkyl-, Alkyloxy-heteroaryl-alkyl-, Alkyloxy-heteroaryl-alkenyl-, Alkyloxy-heterocyclyl-alkyl-, Alkyloxy-heterocyclyl-alkenyl-, Alkyloxy-heterocyclyl-heteroalkyl-, Alkyloxy-heterocyclyl-heteroalkenyl-, Aryl-heteroalkyl-heteroaryl-, aryl-heteroalkenyl-heteroaryl-, Aryl-heteroalkyl-heteroaryl-alkyl-, Aryl-heteroalkenyl-heteroaryl-alkyl-, aryl-heteroalkyl-heterocyclyl-, aryl-heteroaryl-heterocyclyl-; Aryl-alkyl-heterocyclyl Aryl-alkenyl-heterocyclyl, alkenyl-aryl-heteroalkenyl-, Aryl-Alkyl-heterocyclyl-alkyl-, Aryl-Alkyl-heterocyclyl-alkenyl-, Aryl-Alkyl-heterocyclyl-heteroalkyl-, Aryl-Alkyl-heterocyclyl-heteroalkenyl-, Aryl-alkenyl-heterocyclyl-alkyloxy-, Aryl-alkyl-heterocyclyl-alkyloxy-, Aryl-alkenyl-heteroaryl-alkyloxy-, Aryl-alkyl-heteroaryl-alkyloxy-, Aryl-alkyl-heterocyclyl-SO2-, aryl-alkenyl-heterocyclyl-SO2-, heteroaryl-alkyl-heterocyclyl-SO2-, heteroaryl-alkenyl-heterocyclyl-SO2-; aryl-amino, and aryl-NH— and wherein each of said groups can be unsubstituted or substituted with one or more substituents each independently selected from the group comprising halogen, nitro, oxo, alkyloxy, —C(O)OH, —NH₂, hydroxycarbonylalkyl, hydroxycarbonylalkenyl, hydroxyl, alkyl, alkenyl, haloalkyl, haloalkenyl, heteroalkyl, heteroalkenyl ═S, —SH, aryl, heteroaryl, heterocyclyl; aryl-alkyl-; aryl-alkenyl-; arylheteroalkyl-; arylheteroalkenyl-; and heterocyclyl-alkyl-; and
wherein cycle C is selected from the group represented by formula (IIIa);

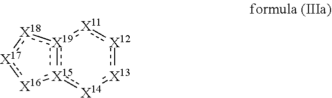

wherein the dotted line represents an optional double bond;
wherein each of X¹⁵ and X¹⁹ is independently selected from N, C, and CH,
wherein each of X¹¹, X¹², X¹³, X¹⁴, X¹⁶, X¹⁷, and X¹⁸ is independently selected from the group comprising N, NH, S, O, C═O, C═S, CH, C(Z⁵)₂ and N(Z⁶), and
   wherein each Z⁵ is independently selected from the group comprising hydrogen, halogen, nitro, hydroxyl, —SH, —NH₂, —C(O)OH, alkyl, heteroalkyl, alkenyl, heteroalkenyl, haloalkyl, aryl, heteroaryl, heterocyclyl, alkyloxy, alkylthio, heterocyclyl-alkyl-, heterocyclyl-alkenyl-, heterocyclyl-heteroalkyl-, heterocyclyl-heteroalkenyl-, aryl-alkyl-, aryl-alkenyl-, aryl-heteroalkyl-; aryl-heteroalkenyl-, heteroaryl-alkyl-, heteroaryl-alkenyl-, heteroaryl-heteroalkyl-, heteroaryl-heteroalkenyl-, heteroaryl-alkyl-heteroaryl-, Heteroaryl-heteroalkyl-heteroaryl-, Aryl-heteroaryl-alkyl-, Aryl-heteroaryl-alkenyl, aryl-heteroaryl-heteroalkyl-, Aryl-heteroaryl-heteroalkenyl, aryl-heteroalkyl-heterocyclyl-, hydroxycarbonylalkyl, and hydroxycarbonylalkenyl; and
   wherein each Z⁶ is independently selected from the group comprising alkyl, alkenyl, haloalkyl, hydroxycarbonylalkyl, hydroxycarbonylalkenyl, heteroalkyl, heteroalkenyl, aryl, heteroaryl, heterocyclyl, alkyloxy, arylalkyl-, arylalkenyl-, arylheteroalkyl-, arylheteroalkenyl-, heteroaryl-alkyl-, heteroaryl-alkenyl-, heterocyclyl-alkyl-, heterocyclyl-alkenyl-, heterocyclyl-heteroalkyl-; heterocyclyl-heteroalkenyl-, heteroaryl-heteroalkyl-, heteroaryl-heteroalkenyl-, heteroaryl-alkyl-heteroaryl-, Heteroaryl-heteroalkyl-heteroaryl-, Aryl-heteroaryl-alkyl-, Aryl-heteroaryl-alkenyl, aryl-heteroaryl-heteroalkyl-, and Aryl-heteroaryl-heteroalkenyl.
or
wherein said modulator is a compound of formula (IV) or an isomer, preferably a stereo-isomer or a tautomer, a solvate, a salt, preferably a pharmaceutically acceptable salt, or a prodrug thereof,

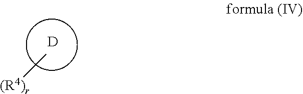

wherein cycle D is selected from the group heteroaryl, aryl, heterocyclyl, and cycloalkyl;
wherein r is an integer selected from 1, 2, 3, 4, 5 or 6; and preferably selected from 1, 2, 3 or 4; and
wherein each R⁴ is independently selected from halogen, nitro, or a group comprising hydroxyl, —NH₂, —C(O)OH, alkyl, alkenyl, haloalkyl, cycloalkyl, cycloalkenyl, heteroalkyl, heteroalkenyl, aryl, heteroaryl, heterocyclyl, arylalkyl-, arylalkenyl-, aryl-heteroalkyl-, aryl-heteroalkenyl-, aryl-heteroaryl-; aryl-heterocyclyl-, alkyl-heteroaryl-, alkenyl-heteroaryl-, heteroalkyl-heteroaryl-, heteroalkyl-heterocyclyl, heteroalkenyl-heteroaryl-, heteroalkenyl-heterocyclyl-, heterocyclyl-alkyl-; heterocyclyl-alkenyl-, heterocyclyl-heteroalkyl-, heterocyclyl-heteroalkenyl-, heterocyclyl-heterocyclyl-, heteroaryl-alkyl-, heteroaryl-alkenyl-, heteroaryl-heteroalkyl-, heteroaryl-heteroalkenyl-, alkyloxy, haloalkoxy, alkenyloxy, aryloxy; heteroaryloxy, heterocylyloxy, alkylthio, alkenylthio, arylthio, heteroarylthio, heterocyclylthio, aryl-alkyloxy-, heteroaryl-alkyloxy-, heterocyclyl-alkyloxy-, aryl-alkylthio-, heteroaryl-alkylthio-, heterocyclyl-alkylthio-, hydroxycarbonylalkyl, hydroxycarbonylalkenyl, alkyl-SO2-, alkenyl-SO2-, heteroalkyl-SO2-, heteroalkenyl-SO2-, aryl-SO2-, heteroaryl-SO2-, heterocyclyl-SO2-, heteroaryl-alkyl-SO2-; heteroaryl-alkenyl-SO2-, heteroaryl-heteroalkyl-SO2-, heteroaryl-heteroalkenyl-SO2-, heteroaryl-heteroalkyl-heteroaryl-, heteroaryl-heteroalkenyl-heteroaryl-, heteroaryl-alkyl-heteroaryl-, heteroaryl-alkenyl-heteroaryl-, Aryl-heteroaryl-alkyl-, aryl-heteroaryl-alkenyl-, aryl-heteroaryl-heteroalkyl-, aryl-heteroaryl-heteroalkenyl-, aryl-heteroalkyl-heteroaryl-; aryl-heteroalkyl-aryl-; aryl-heteroalkyl-heteroaryl-heteroalkyl-; aryl-heteroalkyl-heteroaryl-heteroalkenyl, heteroaryl-heterocylcyl-alkyl, and aryl-NH—, aryl-NH-heteroaryl-heteroalkenyl-, nitroaryl-, nitroaryl-NH—, nitroaryl-NH-heteroaryl-heteroalkenyl-; and wherein each of said groups can be unsubstituted or substituted with one or more substituents each independently selected from the group comprising halogen, nitro, oxo, alkyloxy, —C(O)OH, —NH₂, hydroxycarbonylalkyl, hydroxycarbonylalkenyl, hydroxyl, alkyl, alkenyl, heteroalkyl, heteroalkenyl ═S, —SH, aryl, nitroaryl-, nitroaryl-NH, heteroaryl, and heterocyclyl.
24. The modulator according to any one of aspects 20 to 23, for use in treating infections with antibiotic (multi)resistant bacteria.
25. The modulator according to any one of aspects 20 to 24, for use in treating infections with dormant, latent or persistent bacteria.
26. Use of the crystal structure of the Rel polypeptide as defined by the atomic coordinates presented in any one of Tables 1 to 4, or a subset thereof, or atomic coordinates which deviate from those in any one of Tables 1 to 4, or a subset thereof, by RMSD over protein backbone atoms by no more than 3 Å for designing and/or identifying a compound which modulates Rel hydrolase and/or synthetase activity.
27. A computer system comprising:
   a) a database containing information comprising the atomic coordinates, or a subset thereof as defined in any one of Tables 1 to 4, stored on a computer readable storage medium; and
   b) an user interface to view the information.
28. Use of a computer system as defined in aspect 27 for designing and/or identifying a compound which modulates Rel activity.
29. A crystal of Rel in its unbound resting state, comprising a structure characterized by the atomic coordinates or a subset thereof as defined in Table 1.
30. A crystal of Rel in its synthetase active form, comprising a structure characterized by the atomic coordinates or a subset thereof as defined in Table 2.
31. A crystal of Rel in its hydrolase active form, comprising a structure characterized by the atomic coordinates or a subset thereof as defined in Table 3.
32. A crystal of Rel in its allosteric state, comprising a structure characterized by the atomic coordinates or a subset thereof as defined in Table 4.
33. A method for producing a medicament, pharmaceutical composition or drug, the process comprising: (a) providing a compound according to anyone of aspects 20 to 25 and (b) preparing a medicament, pharmaceutical composition or drug containing said compound.
34. A computer system, intended to generate three dimensional structural representations of a Rel enzyme, Rel enzyme homologues or analogues, complexes of Rel enzyme with binding compounds or modulators, or complexes of Rel enzyme homologues or analogues with binding compounds or modulators, or, to analyse or optimise binding of compounds or modulators to said Rel enzyme or homologues or analogues, or complexes thereof, the system containing computer-readable data comprising one or more of:
   (a) the coordinates of the Rel enzyme structure, listed in any one of Tables 1 to 4, optionally varied by a root mean square deviation of residue backbone atoms of not more than 3 Å, or selected coordinates thereof;
   (b) the coordinates of a Rel enzyme homologue or analogue generated by homology modeling of the target based on the data in (a);
   (c) the coordinates of a candidate binding compound or modulator generated by interpreting X-ray crystallographic data or NMR data by reference to the coordinates of the Rel enzyme structure, listed in any one of Tables 1 to 4, optionally varied by a root mean square deviation of residue backbone atoms of not more than 3 Å, or selected coordinates thereof, and
   (d) structure factor data derivable from the coordinates of (a), (b) or (c).
35. A computer-readable storage medium, comprising a data storage material encoded with computer readable data, wherein the data comprises one or more of
   (a) the coordinates of the Rel enzyme structure, listed in any one of Tables 1 to 4, optionally varied by a root mean square deviation of residue backbone atoms of not more than 3 Å, or selected coordinates thereof;
   (b) the coordinates of a Rel enzyme homologue or analogue generated by homology modeling of the target based on the data in (a);
   (c) the coordinates of a candidate binding compound or modulator generated by interpreting X-ray crystallographic data or NMR data by reference to the coordinates of the Rel enzyme structure, listed in any one of Tables 1 to 4, optionally varied by a root mean square deviation of residue backbone atoms of not more than 3 Å, or selected coordinates thereof, and
   (d) structure factor data derivable from the coordinates of (a), (b) or (c).
36. A computer-readable storage medium comprising a data storage material encoded with a first set of computer-readable data comprising a Fourier transform of at least a portion of the structural coordinates of the Rel enzyme listed in any one of Tables 1 to 4, optionally varied by a root mean square deviation of residue backbone atoms of not more than 3 Å, or selected coordinates thereof; which data, when combined with a second set of machine readable data comprising an X-ray diffraction pattern of a molecule or molecular complex of unknown structure, using a machine programmed with the instructions for using said first set of data and said second set of data, can determine at least a portion of the structure coordinates corresponding to the second set of machine readable data.
37. The computer system according to aspect 34 or computer-readable storage medium according to any one of aspects 35 to 36 further comprising a database containing information on the three dimensional structure of candidate compounds or modulators which are small molecules.
38. A method of treating or preventing infections with antibiotic (multi)resistant bacteria in a subject comprising a Rel modulator as described in aspects 20 to 25, or a pharmaceutical composition comprising a Rel modulator as described in aspects 20 to 25.
39. Use of a Rel modulator as described in aspects 20 to 25, or a pharmaceutical composition comprising a Rel modulator as described in aspects 20 to 25, for the manufacture of a medicament for the prevention or treatment of an antibiotic (multi)resistant bacterial infection.

### BRIEF DESCRIPTION OF THE DRAWINGS

FIG. 1. Biochemical characterization of full-length Rel_{Tt}. (a) Hydrolysis activity of 100 nM full-length Rel_{Tt} was assayed in the presence of 0.5 mM 3H-labeled ppGpp substrate either at 4° C. or 40° C. Synthetic activity of 30 nM full-length Rel_{Tt} was assayed at 40° C. in the presence of either 1 mM ATP (b) or 1 mM APPNP (c), as well 0.3 mM of 3H-labeled GDP and 0.1 mM ppGpp. As indicated on the figure, the reactions were supplemented with 120 nM T. thermophilus 70S IC(MV) added either alone or in combination with 2 μM deacylated E. coli tRNAVal. All experiments were performed in HEPES:Polymix buffer, pH 7.5, 5 mM Mg²⁺, either in the presence (a) or absence (b and c) of 0.5 mM Mn²⁺. Error bars represent Standard deviations of the turnover estimates by linear regression. All the reaction parameters are shown in Table 5.

FIG. 2. Structure of the different Rel_{Tt}^{NTD} catalytic states. (a) Structure of Rel_{Tt}^{NTD} in the resting (nucleotide free) state showing the synthetase domain and hydrolase domain and the α9-α10 α-helical substructure connecting the hydrolase domain and the synthetase domain. (b) Structure of Rel_{Tt}^{NTD} in the active hydrolase state (closed state), bound to ppGpp. (c) Details of the Rel_{Tt}^{NTD}-ppGpp binding interface, all important catalytic residues as well as the Mn²⁺ ion are labeled in the figure. (d) Impact of active site substitutions on the hydrolase activity of Rel_{Tt}, based on the interactions observed in the crystal structure of the Rel_{Tt}^{NTD}-ppGpp complex. In every case substitutions affecting the direct interaction with the guanosine base (R43A), the ribose (Y49F), residues involved in catalysis (D81N, D81E and N150A) or interfering with the accommodation of the phosphate groups of (p)ppGpp (R147G) abrogate hydrolysis. (e) Structure of Rel_{Tt}^{NTD} in the active synthetase state (open state), bound to ppGp_{N}p and AMP. (f) Details of the Rel_{Tt}^{NTD} ppGp_{N}p-AMP binding interface, all important catalytic residues are labeled in the figure with residues directly involved in catalysis shown in bold. (g) Superposition of the SYN-domain of the closed (in light contrast) and open (in dark contrast) states illustrating the rigid body rotation movement of the α9-α10-α13 ‘transmission core’ induced by the binding of nucleotides, that results in the opening of the enzyme. (h) Superposition of the HD-domain of the close (light contrast) and open (dark contrast) states showing the allosteric changes triggered by the activation of the SYN-domain (HD partial occlusion) and the activation of the HD domain (relocation of the α6-α7 snaplock). (i) ppGpp synthetase activity of Rel_{Tt}, and R249A/R277A, R272A/R277A, R249A/R277A/Y329, R272A/R277A/Y329 substituted versions alone, and activated by either T. thermophilus 70S ribosome initiation complex (70S IC) or the 70S IC with deacylated tRNA^{Val} in the A-site.

FIG. 3. Conformations observed in the crystal structure of the unbound Rel_{Tt}^{NTD} (resting state of the enzyme). (a) Lattice packing of the P41212 crystals of free Rel_{Tt}^{NTD}. (b) Representation of the Rel_{Tt}^{NTD} topology with the HD-domain, the α9-α10 linker region and the SYN-domain. (c) Conformation 1, consistent with most of the conformations observed in the catalytic domains of Rel enzymes. (d) In conformation 2 both catalytic domains are arranged as in conformation 1, however the α6-α7 motif (labeled in the figure) protrudes away from the structure and is stabilized by lattice contacts. (e) Superposition of the catalytic domains of different Rel-like enzymes in the resting state (from T. thermophilus, M. tuberculosis and S. dysgalactiae. From the superposition it becomes apparent that the α6-α7 α-helical motif of the hydrolase domain accounts for the main differences in conformation between these structures. (f) Detailed view of the α6-α7 α-helical motif as in (b) but now including α6-α7 from Mesh1 a constitutively active ppGpp hydrolase from H. sapiens. (g) The alternative conformation observed in the lattice, shown in panel (c), is most likely the result of the lattice constrains on the fold. Nevertheless it underscores the dynamic nature of this catalytic domain and, this dynamic interplay involving the ppGpp binding site in the HD-domain must have a strong impact in catalysis.

FIG. 4. Electron density map representation (unbiased mFo-DFc), of the hydrolase domain RelTtNTD as observed in the closed form bound to ppGpp, after refinement with Buster/TNT. The map was calculated from the MR solution omitting the ligand.

FIG. 5. (a) Structure of the Rel_{Tt}^{NTD}-ppGpp complex (shown in light contrast) superimposed on the Rel_{Seq}^{NTD}-ppG2′:3′p complex, shown in strong contrast (b). Structure of the Rel_{Tt}^{NTD}ppGpp complex (light contrasting) superimposed on the Mesh1-NADP complex (shown in strong contrast). The comparison reveals a conserved active site architecture held together by the presence of a Mn²⁺ ion that coordinates residues from three different α-helices (α3, α4 and α8) and is directly involved in catalysis. In addition these superpositions suggest a crucial role for α-helices α6 and α7 in the accommodation and stabilization of the substrate in the active site. (c) Surface representation of Rel_{Tt}^{NTD}-ppGpp. The nucleotide binding site (for ppGpp or pppGpp) is traced in black dashed lines, highlighting the peculiar surface electrostatics of the active site with one acid half involved directly in hydrolysis and positive half involved in the stabilization of the large number of phosphate groups carried by (p)ppGpp.

FIG. 6. Allosteric rearrangements and active site reshaping of Rel_{Tt}^{NTD} as a function of nucleotides (colored as in FIG. 2b). (a) The hydrolase active site in the catalytically compatible form observed in the Rel_{Tt}^{NTD}-ppGpp complex forms an L-shape crevice to accommodate (p)ppGpp (shown as a solid black volume). The allosteric arrangements involved in the active site setup are couple to the closing of the enzyme that constricts the synthetase active site (shown as a light contrasting solid volume) and prevents ppGpp synthesis. (b) Analysis of the hydrolase (solid symbols) and synthetase active site (open symbols) dimensions from the structures complexes of Rel_{Tt}^{NTD}-ppGpp and Rel_{Tt}^{NTD}-ppGpNp-AMP. In the Rel_{Tt}^{NTD}-ppGpp complex the HD active site is larger and 2 Å broader on average than when the enzyme is in the active synthetase form in contrast with what is observed in the synthetase site which becomes much larger and significantly broader in the Rel_{Tt}^{NTD}-ppGp_{N}p-AMP compared with the closed form. (c) Active site representation of the enzyme in the open form (Rel_{Tt}^{NTD}-ppGp_{N}p-AMP complex) shown as in (a). The figure shows the stretching of the two catalytic domains involved in the correct arrangement of the synthetase catalytic site which is coupled to the closing and inactivation of the hydrolase domain catalytic site.

FIG. 7. (a) Electron density map representation (unbiased mFo-DFc), of the synthetase domain Rel_{Tt}^{NTD} as observed in the open form bound to ppGp_{N}p and AMP (ball-and-stick models), after refinement with Buster/TNT. The map was calculated from the MR solution omitting the ligand. (b) Superposition of the open and closed conformations of Rel_{Tt}^{NTD} displaying the different positions of the α9-α10-α13 ‘transmission core’. The rigid body swivel of the transmission core triggers the opening of the enzyme and partial occlusion of the HD-domain active site. (c) Details of the conformational rearrangement of the transmission core induced by the binding of nucleotides.

FIG. 8. (a) Superposition of the active site residues of Rel_{Tt}^{NTD} in the PC state on the active site residues of the RelP small alarmone syntetase in a pre-catalytic state (bound to GTP and APCPP). The catalytic residues of Rel_{Tt}^{NTD} are shown in bold and the equivalent residues of RelP in regular font. The G-loop that stabilizes the guanosine group of GTP/GDP and the product (p)ppGpp is labeled as well as the amphipathic α-helix α13 that contains a positive surface that coordinates the pyrophosphate group that is transferred from ATP to GDP or GTP. The comparison shows a remarkably conserved active despite both domains having less than 20% sequence identity. (b) Same as in (a) but displaying the ligands observed bound in the active site of both complexes; ppGp_{N}p and AMP in the case of the post-catalytic state complex of Rel_{Tt}^{NTD} and GTP and AMP in the case of the precatalytic state complex of RelP. From the structural comparison it becomes apparent that both enzymes most accommodate the two substrates in the same way with AMP and APCPP bound in almost the same position and orientation and ppGp_{N}p accommodated with a small rearrangement of the G-loop likely to compensate for the lack of the extra phosphate group present in the GTP molecule. The catalytic residues D272, E345 and Q347 suggested to be involved in the hydrolysis and transferring of the pyrophosphate group are labeled together with the G-loop, α13 and the αP group of AMP.

FIG. 9. Rel_{Tt}^{NTD} conformational dynamics in the presence of nucleotides assessed by smFRET. (a) Structural models of Rel_{Tt}^{NTD} in the open and closed conformations (to probe the inter-domain movements associated with the nucleotides allosteric control). Dye attachment sites are Leu6Cys (L6) and Thr287Cys (T287). 1D projection histograms of the FRET efficiency of the Rel_{Tt}^{NTD}_{6/287} in the presence of ppGpp (b), GDP+APCPP (c), APCPP (d), GDP (e) and GDP+ATP (f). The analysis of the smFRET data suggest that the high FRET state of the enzyme in the presence of ppGpp and APCPP is consistent with the closed form observed in the crystal structure of the complex with ppGpp. By contrast the low FRET state of the enzyme observed in the presence of GDP and GDP+APCPP is consistent with the open state of the enzyme observed in the structure of Rel_{Tt}^{NTD}-ppGpNp-AMP. (g) Titration of GDP into Rel_{Tt}^{NTD}. (h) Titration of APCPP into Rel_{Tt}^{NTD} in the presence of saturating amounts of GDP. (i) Structural models of Rel_{Tt}^{NTD} in the open and closed conformations. Dye attachment sites are Leu6Cys and Thr124Cys (to probe HD-domain movements associated with the nucleotides allosteric control). 1D projection histograms of the FRET efficiency of the RelTtNTD6/124 in the presence of ppGpp (j), GDP+APCPP (k) and ppGpp+EDTA (l). The lower FRET state of the hydrolase domain in the presence of ppGpp is consistent with the movement of α-helices α6 and α7 which allow the binding of the substrate into the active site. By contrast the higher FRET state observed in the presence of GDP+APCPP is compatible with the snaplock switch that closes the hydrolase state coupled to the activation of the synthetase domain and the overall stretching of both catalytic domains.

FIG. 10. Single molecule FRET analysis. Two-dimensional (2D) histograms of the FRET efficiency E versus the stoichiometry S and PDA analysis of Rel_{Tt}^{NTD}_{6/287} in the presence of ppGpp (a), GDP+APCPP (b), APCPP (c), GDP (d) and GDP+ATP (e). Two-dimensional (2D) histograms of the FRET efficiency E versus the stoichiometry S and PDA analysis of Rel_{Tt}^{NTD}_{6/124} in the presence of ppGpp (f), APCPP (g) and ppGpp+EDTA (h).

FIG. 11. ITC measurements. (a) Titration of APCPP into Rel_{Tt}^{NTD}. (b) Theoretical thermodynamic parameters involved in the binding of APCPP to Rel_{Tt}^{NTD} obtained from the differences between the binding of APCPP to Rel_{Tt}^{NTD}-GDP and the interaction of Rel_{Tt}^{NTD} with GDP. All the thermodynamic parameters associated with each titration are listed in Table 6.

FIG. 12. Cartoon representation of the molecular model for the mechanism of regulation of Rel_{Tt}^{NTD} catalysis as a function of nucleotides. In absence of ligands the resting state of the enzyme is consistent with the conformations observed in RelA_{Mtb}^{NTD} and RelA_{Ec} with the catalytic site of the SYN-domain partially occluded (the ATP binding site completely buried in the resting state). The ppGpp synthesis cycle is initiated by GDP binding which triggers an open form that allows the consequent binding of ATP, followed by the synthesis of ppGpp. Moreover, the stabilization of this open state is coupled to changes in the active site of the HD-domain that preclude hydrolysis. Conversely, ppGpp binding to the HD-domain triggers the overall closing of the enzyme resulting in the complete occlusion of the active site of the SYN-domain safe-guarding against non-productive ppGpp synthesis and the alignment of the active site residues in the HD-domain, to accommodate ppGpp and catalyze the hydrolysis of the 3′-pyrophosphate group of the molecule.

FIG. 13. Putative novel site that stabilizes the enzyme in a resting-like inactive state. (a) Superposition of different Rel catalytic domains. (b) S. aureus Rel hydrolase active. (c) Structure of the catalytic region of S. aureus Rel bound to GDP (with GDP in the synthetase domain). (d) Electrondensity corresponding to the GDP molecule observed in the active site of S. aureus Rel. Electrondensity corresponding to the pppGpp molecule observed in the active site of S. aureus Rel hydrolase domain (e) and synthetase domain (f). Additional pppGpp molecule (pppG2′:3′p) observed in the structure of S. aureus Rel bound to pppGpp, at interface between both catalytic domains (g).

FIG. 14. Putative novel site that stabilizes the enzyme in a resting-like inactive state. (a) Domain representation of a long RSH enzyme such as Rel or RelA. (b) Cartoon representation of the secondary structure of the catalytic domains of Rel/RelA. (c) Structure of the catalytic region of S. aureus Rel unbound. (d) Structure of the catalytic region of S. aureus Rel bound to pppGpp. (e) Highlight of the interactions of pppGpp in the hydrolase active site of S. aureus Rel. (f) Highlight of the interactions of pppGpp in the synthetase active site of S. aureus Rel.

### DETAILED DESCRIPTION OF THE INVENTION

As used herein, the singular forms “a”, “an”, and “the” include both singular and plural referents unless the context clearly dictates otherwise.

The terms “comprising”, “comprises” and “comprised of” as used herein are synonymous with “including”, “includes” or “containing”, “contains”, and are inclusive or open-ended and do not exclude additional, non-recited members, elements or method steps. The terms also encompass “consisting of” and “consisting essentially of”, which enjoy well-established meanings in patent terminology.

The recitation of numerical ranges by endpoints includes all numbers and fractions subsumed within the respective ranges, as well as the recited endpoints.

The terms “about” or “approximately” as used herein when referring to a measurable value such as a parameter, an amount, a temporal duration, and the like, are meant to encompass variations of and from the specified value, such as variations of ±10% or less, preferably ±5% or less, more preferably ±1% or less, and still more preferably ±0.1% or less of and from the specified value, insofar such variations are appropriate to perform in the disclosed invention. It is to be understood that the value to which the modifier “about” refers is itself also specifically, and preferably, disclosed.

Whereas the terms “one or more” or “at least one”, such as one or more members or at least one member of a group of members, is clear per se, by means of further exemplification, the term encompasses inter alia a reference to any one of said members, or to any two or more of said members, such as, e.g., any 3 or more, 4 or more, 5 or more, 6 or more, or 7 or more etc. of said members, and up to all said members. In another example, “one or more” or “at least one” may refer to 1, 2, 3, 4, 5, 6, 7 or more.

The discussion of the background to the invention herein is included to explain the context of the invention. This is not to be taken as an admission that any of the material referred to was published, known, or part of the common general knowledge in any country as of the priority date of any of the claims.

Throughout this disclosure, various publications, patents and published patent specifications are referenced by an identifying citation. All documents cited in the present specification are hereby incorporated by reference in their entirety. In particular, the teachings or sections of such documents herein specifically referred to are incorporated by reference.

Unless otherwise defined, all terms used in disclosing the invention, including technical and scientific terms, have the meaning as commonly understood by one of ordinary skill in the art to which this invention belongs. By means of further guidance, term definitions are included to better appreciate the teaching of the invention. When specific terms are defined in connection with a particular aspect of the invention or a particular embodiment of the invention, such connotation is meant to apply throughout this specification, i.e., also in the context of other aspects or embodiments of the invention, unless otherwise defined.

In the following passages, different aspects or embodiments of the invention are defined in more detail. Each aspect or embodiment so defined may be combined with any other aspect(s) or embodiment(s) unless clearly indicated to the contrary. In particular, any feature indicated as being preferred or advantageous may be combined with any other feature or features indicated as being preferred or advantageous.

Reference throughout this specification to “one embodiment”, “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention, and form different embodiments, as would be understood by those in the art. For example, in the appended claims, any of the claimed embodiments can be used in any combination.

Amino acids are referred to herein with their full name, their three-letter abbreviation or their one letter abbreviation.

The following detailed description is not to be taken in a limiting sense. The scope of the present invention is defined by the appended claims. It is evident that disclosed embodiments may relate to both RSH enzyme modulators such as Rel modulators and methods to identify Rel and/or RSH enzyme modulators. Certain embodiments directed to the Rel and/or RSH modulators may apply to the methods or uses described herein. It is evident that the terms such as “(candidate) compound”, “(candidate) binding compound”, and “(candidate) ligand” may be used interchangeably to describe the invention.

A skilled person is aware of standard molecular biology techniques that are available in the art (Sambrook et al., Molecular cloning: a laboratory manual, Cold Spring Harbor laboratory press, 1989; Ausubel et al., Current protocols in molecular biology, John Wiley and Sons, 1989; Perbal, A Practical Guide to Molecular Cloning, John Wiley & Sons, 1988; Watson et al., Recombinant DNA, Scientific American Books, New York; Birren et al. Genome Analysis: A Laboratory Manual Series, Vols. 1-4 Cold Spring Harbor laboratory press, New York, 1998).

The term “RSH enzymes” as used herein is an abbreviation for the group of RelA/SpoT homolog enzymes. RSH enzymes derive their name from the sequence similarity to the RelA and SpoT enzymes of Escherichia coli. RSH enzymes comprise a family of enzymes that synthesize and/or hydrolyze the alarmone ppGpp and play a central role in the bacterial stringent response. So-called “Long” RSH enzymes that comprise a hydrolase and synthetase domain have been identified in a vast and diverse amount of bacteria and plant chloroplasts, while specific RSH enzymes that only synthesize or hydrolyze (p)ppGpp have also been discovered in disparate bacteria and animals respectively. In the art, RSH enzymes are stratified into three groups based on their activity: long RSH enzymes, small alarmone synthetases (SASs), and small alarmone hydrolases (SAHs). These initial groups have been further classified in a plethora of subgroups (Atkinson et al., The RelA/SpoT Homolog (RSH) Superfamily: Distribution and functional evolution of ppGpp synthetases and hydrolases across the tree of life, Plos One, 2011). Long RSHs comprise two catalytic domains (the (p)ppGpp hydrolase (HD) domain and the (p)ppGpp synthetase (SYN) domain) and a C-terminal protein domain that is involved in regulation of the enzyme. In contrast, both SASs and SAHs lack the conserved C-terminal regulatory domain. According to the art, long RSHs are most broadly distributed and often further comprise TGS (ThrRS, GTPase, and SpoT) and ACT (Aspartokinase, Chorismate mutase and TyrA) domains in their C-terminal domain, which may play a role in sensing stress signals such as starvation signals and transducing said signal to the catalytic domain. The “Rel protein” or “Rel enzyme” as referred to herein is an example of a bifunctional RelA/SpoT homolog that is able to both synthesize and hydrolyse (p)ppGpp. Hence, Rel is able to control (p)ppGpp levels by its opposing activities. Structural similarities have been described between the Rel hydrolase domain and the 3′,5′-cyclic-nucleotide phosphodiesterase superfamily (Enzyme commission number (E.C. number) 3.1.4.17), as well as between the Rel synthetase domain and the nucleotidyltransferase superfamily (E.C. 2.7.7.-), particularly DNA polymerase β (Hogg et al., Conformational Antagonism between Opposing Active Sites in a Bifunctional RelA/SpoT Homolog Modulates (p)ppGpp Metabolism during the Stringent Response, Cell, 2004).

The term “small” as used as used herein, e.g. in terms such as “small molecule” or “small compound” or “small candidate (binding) compound” refers to a low molecular weight compound that is organic, anorganic or organometallic and has a molecular weight of less than 1000 Da, and for instance has a molecular weight of less than 900 Da, or less than 750 Da, or even less than 600 Da. Small compounds used in the methods herein may be naturally occurring or solely occurring due to chemical synthesis.

The term “stringent response”, used interchangeably in the art with “stringent control” is indicative for a stress response mediated by RSH enzymes in response to various stress conditions including the non-limiting examples of amino acid starvation, fatty acid limitation, iron limitation, and heat shock. In such stress conditions, the stringent response mediates a profound shift in gene expression from a program focused on growth to a gene expression profile that allows prolonged survival in a stationary phase following failure of aminoacyl-tRNA pools to support protein synthesis. Hence, the stringent response is a key mediator in the process of bacterial persister cell formation. The stringent response has been extensively described in the art (inter alia in Traxler et al., The global, ppGpp-mediated stringent response to amino acid starvation in Escherichia coli, Molecular microbiology, 2013). The stringent response is governed by the alarmones guanosine 5′,3′ bispyrophosphate and guanosine pentaphosphate (ppGpp and pppGpp respectively). (p)ppGpp accumulation will actively inhibit resource intensive cellular processes including replication, transcription and translation. (p)ppGpp has been demonstrated to bind to RNA polymerase proximal to its active site which causes a cessation of transcription of stable RNAs. Furthermore, (p)ppGpp decreases the half-life of the open complex at most promoters that have been tested in the art, hereby mediating a strong down regulation of promoters with intrinsically short half-lives, such as those of stable RNA genes. Taken together, the stringent response includes a large-scale down regulation of the translation apparatus (Barker et al., Mechanism of regulation of transcription initiation by ppGpp. Effects of ppGpp on transcription initiation in vivo and in vitro, Journal of molecular biology, 2001). Additionally, (p)ppGpp has been shown to upregulate transcription of promoters that act on amino acid biosynthesis genes together with RNA-polymerase binding transcription factor DksA (Paul et al., DksA potentiates direct activation of amino acid promoters by ppGpp, PNAS USA, 2005).

“Persister cells”, or short “persisters” as used herein is used to describe a population of bacterial cells that are in or going into a metabolically inactive (i.e. dormant) or near dormant state characterized by no growth or very slow growth, also called a stationary phase (Lewis, Persister cells, dormancy and infectious disease, Nature reviews microbiology, 2007). Typically, in an infected organism which is optionally being treated with antibiotics, persister cells amount to a small fraction of the total bacterial population present in said infected organism. Upon termination of antibiotics treatment, persister cells can leave their dormant state and return to a growth-focused gene expression signature, and expand to a full size bacterial infection. Persister cells are often described to constitute a subpopulation of bacteria that, due their slow growth rate, become highly tolerant to antibiotics. Persistent bacterial cells are not per definition originating from genetic mutation, although a skilled person is aware that persistence of a bacterial cell is associated with the emergence of antibiotic resistance (Windels et al., Bacterial persistence promotes the evolution of antibiotic resistance, 2019). Links between (p)ppGpp production and formation of bacterial persister cells have been described (inter alia in Korch et al., Characterization of the hipA7 allele of Escherichia coli and evidence that high persistence is governed by (p)ppGpp synthesis, Molecular microbiology, 2003). Persister cells may form within biofilms.

The term “biofilm” is commonly used in the art and is indicative for a collection or aggregate of (syntrophic) microorganisms such as bacteria wherein the different cells adhere to each other, and optionally the surface contacting the cells, or a portion of the cells. Biofilms are further characterized by a viscous extracellular matrix comprising extracellular polymeric substance (EPS) produced by microorganisms of the biofilm, wherein the microorganisms are embedded by the EPS. Biofilms may be formed both in or on organisms and on non-living surfaces in a wide array of different settings. Biofilms are complex microbiological systems wherein the microorganism comprised in said biofilm may be organized into a functional unit or functional community (Lopez et al., Biofilms, Cold Spring Harbor perspectives in biology, 2010).

The term “alarmones” is known to a skilled person and refers to intracellular signal molecules that are produced as a consequence of and in response to environmental cues. The main function of alarmones is to regulate gene expression. Typically, the concentration of alarmones rises when a cell experiences stressful environmental factors. (p)ppGpp is considered a textbook example of an alarmone (Hauryliuk et al., Recent functional insights into the role of (p)ppGpp in bacterial physiology, Nature reviews microbiology, 2015).

“Modulator” as used herein indicates a molecule that influences one or more (enzymatic) activities of one or more proteins upon interaction with (and/or binding of) said protein. As used herein, the modulating effect of the modulators described herein is intended to act on the hydrolase activity, or the synthetase activity, or both the hydrolase and synthetase activity of the Rel protein as defined herein. The principal binding site of a modulator is commonly termed the orthosteric site, which may be for example the active site of an enzyme where it engages in a binding with (a) substrate(s). Additionally, modulators may exert their activity by binding to a second binding site, commonly referred to as an allosteric binding site. In instances where a modulator impacts both the hydrolase and the synthetase activity, the direction of activity modulation is not limited to upregulation or downregulation of both enzymatic activities, but can also entail an upregulation of one enzymatic activity and downregulation of the other (e.g. upregulation of hydrolase activity and downregulation of synthetase activity by a single modulator, or vice versa). Hence, a modulator as discussed herein can refer to a molecule that is a hydrolase activator, hydrolase inhibitor, synthetase activator, or synthetase inhibitor, or any one or more of these. In certain embodiments, the molecule modulates the activity level of one enzymatic domain or enzymatic moiety but induces no significant effect on the activity level of the other enzymatic domain or enzymatic moiety.

“Synthetase” as used herein refers to an enzyme, or enzyme domain that catalyses a synthesis process. In the context of the invention, “synthetase activity” refers to the transfer of pyrophosphate from ATP to the 3′ position of the ribose of GDP or GTP. The term “hydrolase” used herein is indicative for a class of enzymes or enzyme domains that utilise water to disrupt, or break a chemical bond, generating two distinct molecules from one molecule. Hence, it is evident that hydrolase refers to an enzyme capable of conducting hydrolysis. Unless explicitly mentioned, by hydrolase activity herein is meant the hydrolysis of (p)ppGpp, i.e. removal of the 3′ pyrophosphate moiety from (p)ppGpp. Long RSH enzymes such as Rel are known to comprise both the synthetase and hydrolase activity described above and are thus able to both synthesize and degrade alarmones (i.e. (p)ppGpp).

In the context of the invention, a modulator is said to be an “inhibitor” when a consequence of interaction between the modulator and the target protein, here RSH enzymes such as the Rel protein, is that at least one activity of said target protein is reduced, either partially (i.e. to a certain degree) or completely. In the latter case it is understood that due to interaction with the modulator an enzymatic activity of the target protein is diminished to 0%, or below an activity level that can be measured by methods available in the art (such as in Gratani et al., Regulation of the opposing (p)ppGpp synthetase and hydrolase activities in a bifunctional RelA/SpoT homologue from Staphylococcus aureus, PLoS genetics, 2018). “Inhibition” as used herein refers to the inhibition of a process, herein a molecular process, more particularly either the RSH or Rel enzyme hydrolase activity, synthetase activity, or both. It is evident to a skilled person that inhibition can be used interchangeably with the term “attenuation”. In certain embodiments, the inhibitor selectively inhibits Rel hydrolase activity. In alternative embodiments, the inhibitor selectively inhibits Rel synthetase activity. In yet alternative embodiments, the inhibitor selectively inhibits both Rel hydrolase and synthetase activity. Both reversible and irreversible inhibitors are envisaged herein. “Reversible inhibition” and “irreversible inhibition” are known terms to person skilled in the art and are commonly used to further specify an enzyme inhibitor. Binding of an inhibitor to an enzyme is either reversible or irreversible. Irreversible inhibitors usually react with the enzyme and induce a chemical change or modification (e.g. via covalent bond formation). These inhibitors typically modify key amino acid residues needed for enzymatic activity. In contrast, reversible inhibitors bind non-covalently and different types of inhibition have been described depending on whether these inhibitors bind to the enzyme, the enzyme-substrate complex, or both. Methods to measure the dissociation constant (K_{d}) of a reversible inhibitor are well known to a skilled person (Pollard, A guide to simple and informative binding assays, Molecular biology of the cell, 2010).

The term “dissociation constant”, or “K_{d}” used herein is an equilibrium constant that quantitatively expresses the propensity of a larger object to separate or dissociate reversibly into smaller components. It is known to a person skilled in the art that the dissociation constant is routinely used to quantify the affinity between a ligand and a drug and is therefore indicative for how tightly or strongly a ligand binds to its target protein. The affinity of a ligand for a protein is associated with the amount of non-covalent intermolecular interactions between the ligand and the protein such as hydrogen bonds, electrostatic interactions, hydrophobic interactions and Van der Waals forces. In addition, the concentration of other molecules present in the proximal environment the ligand-protein interaction takes place in can also affect affinities. This observation is known to a skilled person as molecular crowding (Rivas et al., Macromolecular crowding in vitro, in vivo, and in between, Trends in biochemical sciences, 2016).

“In silico analysis” as defined herein is indicative for an analysis performed on a computing system or by use of a computer simulation system that is guided by a set of specific instructions such as a molecular docking computer program or tool. “Molecular docking” indicates a method that allows prediction of a binding and/or preferred orientation of one molecule to a second molecule when bound to each other to form a stable complex. Hence, it is understood that molecular docking software predicts the behaviour of molecules in binding sites of target proteins. Molecular docking software tools and programs that allow assessing of specificity of a candidate molecule or candidate compound against a particular target have been described in the art. Molecular docking software allows searching for complementarities between shape and/or electrostatics of binding sites surfaces and ligands. A molecular docking process can be separated into two major steps: searching and scoring. Numerous examples of different docking tools and programs have been described and are thus known to a skilled person (Pagadala et al., Software for molecular docking: a review, Biophysical Reviews, 2017). Two main popular molecular docking approaches have been described, a first being molecular docking relying on shape complementarity or geometric matching, and a second one relying on simulating the docking process whereby ligand-protein pairwise interaction energies are calculated.

A “conformational change” as described herein is to be understood as a change in the three-dimensional shape of a molecule, here an RSH enzyme such as Rel. A conformational change may be induced by numerous factors including the non-limiting examples of temperature, pH, voltage, light, ion concentration, post translational modification or binding to a second molecule. The conformational change as described in the current application is a consequence, either directly or indirectly, of binding to a modulator molecule. A protein may display different functions and/or engage in distinct interactions depending on its conformation. In light of the current invention, the conformational state may impact the hydrolase and/or synthetase activity levels. In certain embodiments, specific conformations partially or even completely inhibit hydrolase and/or synthetase activity. In alternative embodiments, specific conformations cause an upregulation of the hydrolase and/or synthetase activity. When “stabilization” of a conformational state is described in the context of the current invention upon binding a Rel modulator, it is intended that the Rel protein adopts a particular state such as but not limited to an open or closed state for at least the time window wherein candidate compound-Rel interaction is occurring.

The term “crystal structure” as used herein is a three-dimensional description of ordered arrangements or structures of elements such as atoms, ions, or molecules in a crystalline material. Crystal structure refers to a protein crystal structure obtained by protein crystallography, the process of forming a protein crystal by experimentation, unless stated otherwise. In a typical protein crystallization process, proteins are dissolved in an aqueous environment comprising a sample solution until supersaturation is obtained. Different approaches have been described in detail in the art and include as non-limiting examples vapor diffusion, batch, microdialysis and liquid-liquid diffusion. Once a protein crystal is obtained, different techniques such as X-ray diffraction, cryo-electron microscopy, or nuclear magnetic resonance are suitable to determine the protein crystal structure. The term “supersaturation” refers to a condition of a solution that contains more of a dissolved material than can be dissolved by the solvent under normal conditions and has been defined in the art as a non-equilibrium condition in which some quantity of the macromolecule in excess of the solubility limit, under specific chemical and physical conditions, is nonetheless present in solution (McPherson and Gavira, Introduction to protein crystallization, Structural biology communications, 2014). Protein crystals thus also compose a large amount of solvent molecules such as the non-limiting example of water. Due to the different methodologies for preparing a protein crystal, these crystals further comprise a varying range of buffers, salts, small binding proteins, and precipitation agents which can vary substantially in concentration.

Typical crystals have a size of between 20 μm to multiple mm. A crystal optimal for X-ray diffraction analysis is ideally free of cracks and other defects.

In certain embodiments, the amino acid sequence of Rel as used by the (screening) methods described herein has at least 70%, preferably at least 80% sequence identity to the amino acid sequence of the Thermus thermophilus RelA/SpoT (P)ppGpp synthetase I as defined in SEQ ID NO: 1:

>tr|A0A1J1EKV2|A0A1J1EKV2_THETH (P)ppGpp
synthetase I SpoT/RelA OS = Thermus thermophilus
OX = 274 GN = TTMY_1322 PE = 3 SV = 1
MVGADLGLWNRLEPALAYLAPEERAKVREAYRFAEEAHRGQLRRSGEPYI
THPVAVAEILAGLQMDADTVAAGLLHDTVEDCGVAPEELERRFGPTVRRI
VEGETKVSKLYKLANLEGEERRAEDLRQMFIAMAEDVRIIIVKLADRLHN
LRTLEHMPPEKQKRIAQETLEIYAPLAHRLGMGQLKWELEDLSFRYLHPE
AFASLSARIQATQEARERLIQKAIHLLQETLARDELLQSQLQGFEVTGRP
KHLYSIWKKMEREGKTLEQIYDLLAVRVILDPKPAPTRESQALREKQVCY
HVLGLVHALWQPIPGRVKDYIAVPKPNGYQSLHTTVIALEGLPLEVQIRT
REMHRVAEYGIAAHWLYKEGLTDPEEVKRRVSWLKSIQEWQKEFSSSREF
VEAVTKDLLGGRVFVFTPKGRIINLPKGATPVDFAYHIHTEVGHHMVGAK
VNGRIVPLSYELQNGEIVEILTSKNAHPSKGWLEFAKTRSAKSKIRQYFR
AQERQETLERGQHLLERYLKRRGLPKPTDSQLEEAAKRLSLPPSPEELYL
ALALNRLTPRQVAEKLYPKALLKPEKPKPQVRNEWGIRLEQDLQAPIRLA
SCCEPMKGDPILGFVTRGRGVTVHRADCPNLRRLLQGPEADRVIGAYWEG
VGGKVVVLEVLAQDRAGLLRDVMQVVAEAGKSALGSETRVLGPLARIRLR
LAVQDGEEERILQAVQKVSGVKEARWA

In certain embodiments, the amino acid sequence of a Rel as used by the (screening) methods described herein has preferably at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, at least about 99.5% sequence identity to the amino acid sequence defined in SEQ ID NO: 1. In certain embodiments, the amino acid sequence of a Rel as used by the (screening) methods described herein is as defined in SEQ ID NO: 1.

In a first aspect, the invention relates to a method for identifying compounds that modulate Rel hydrolase and/or Rel synthetase activity comprising the step of employing a three dimensional structure represented by a set of atomic coordinates presented in Table 1, 2, 3, or 4 or a subset thereof, or atomic coordinates which deviate from those in Table 1, 2, 3, or 4, or a subset thereof, by RMSD over protein backbone atoms by no more than 3 Å and assessing the degree of fit to the three-dimensional protein structure of Rel of said candidate compound. “RMSD”, “root-mean-square deviation”, or “root-mean-square deviation of atomic positions” as used herein is indicative for a quantitative measurement of similarity between two or more protein structures, more specifically the measure of the average distance between the (backbone) atoms of superimposed proteins. The RMSD value is commonly calculated by the formula:

$$R MSD = √ ( ( \frac{1}{N} ) {\sum }_{i = 1}^{N} {δ}_{i}^{2}$$

wherein δ is the distance between atom i and the mean position of the N equivalent atoms, or alternatively a reference structure. When calculating the RMSD for backbone, heavy atoms values are calculated for C, N, O, and C_{α} or solely for C_{α}. As a RMSD value represents a distance, the value is commonly expressed in the art in Å (Angstrom). 1 Å corresponds to 10⁻¹⁰ m, or 0.1 nanometer. A skilled person appreciates that a lower RMSD indicates smaller structural differences between the compared structures, or between a structure and a reference structure. In certain embodiments, the atomic coordinates used in the method deviate by no more than 2.5 Å, preferably no more than 2 Å, more preferable no more than 1.5 Å, even more preferably no more than 1 Å from the atomic coordinates of Table 1, 2, 3, or 4. In certain embodiments where a subset of atomic coordinates is used, the subset of coordinates is uniquely present in Table 1, 2, 3, or 4. In alternative embodiments where a subset of atomic coordinates is used, the subset of coordinates is present in each of Table 1, 2, 3, and 4.

The term “atomic coordinates” as used herein refers to a position of an atom in space, typically expressed by a set of X, Y, and Z Cartesian coordinates and the chemical element each atom represents. Atomic coordinates for a certain protein structure are typically combined in atomic coordinate data files, which can have various data formats, including the formats of Tables 1, 2, 3, or 4 as enclosed in this specification. Other non-limiting data formats include Protein Data Bank (PDB) format or various text formats. Minor variations in the atomic coordinates are envisaged, and the claims have been formulated with the intent of encompassing such variations. In certain embodiments, the atomic coordinates further contain additional information. It is evident to a skilled person that a three-dimensional rigid body rotation or a translation of said atomic coordinates does not alter the structure of the molecule. It is evident that, since the atomic coordinates disclosed herein are a relative collection of points delineating a three-dimensional structure, a distinct set of coordinates may define a similar or identical three-dimensional structure. In view hereof, multiple computer analysis tools and programs have been developed to assess whether a molecular structure bears similarity to the structured defined by the atomic coordinates, or a subset of atomic coordinates described herein in Table 1, 2, 3, or 4. By means of illustration and not limitation, a suitable software application for conducting such analyses is the Molecular Similarity program of QUANTA (Molecular Simulations Inc., San Diego, Calif.). The Molecular Similarity program and consorts permit extensive comparison between different structures, different conformations of the same structure, and different parts of the same structure. The method of comparison typically involves a step of calculating one or more optimal translations and rotations required such that the RMSD of the fit over the specified pairs of equivalent atoms is an absolute minimum. Therefore, atomic coordinates of a Rel protein or fragments leading to the atomic coordinates in any of Tables 1, 2, 3, or 4 by translations and/or rotations are within the scope of the present invention.

“Degree of fit”, or alternatively “goodness of fit” in the art, is an expression to indicate the likelihood that a certain candidate binding mode represents a favourable binding interaction and allows ranking of different ligands relative to each other. In certain embodiments, the degree of fit between the three-dimensional Rel structure and the candidate Rel modulator is expressed with a numerical value. In alternative embodiments, the degree of fit is expressed by an illustration of the superimposed Rel structure and the compound structure. In certain embodiments, the degree of fit of a ligand is expressed relative to the fit of a known ligand of the Rel protein. A degree of fit may be expressed as an absolute or relative value, depending on the template used for calculating the quantitative score. When the degree(s) of fit are expressed as absolute values, this absolute value corresponds to a score given to a candidate compound based on the number of interactions in silico predicted to occur with a set of atomic coordinates as described in Tables 1 to 4 herein, and/or with a set of amino acid residues in said region on the surface of the protein as described herein. Said number of interaction can be one or more such as two, three, four, five, six, seven, eight, nine, ten, more than ten, or all amino acid residues in said region on the surface of the protein as defined herein. In certain embodiments, the atomic coordinates described in Tables 1 to 4, and/or the amino acid residues cited herein to constitute a surface region of the protein are further abstracted to a pharmacophore, i.e. a set of molecular features required for molecular recognition of a ligand by a biological macromolecule, herein the candidate compound and the Rel protein, Rel hydrolase, or Rel synthetase domain. In certain embodiments, a degree of fit (i.e. a fitting score) of 2.4 is used as threshold for candidate compounds to be considered for further examination and/or validation. In alternative embodiments, a fitting score of 3.0 is used. In alternative embodiments, a fitting score of between 2.4 and 3.0 is used, preferably between 2.5 and 3.0, between 2.7 and 3.0, between 2.9 and 3.0. In alternative embodiments a fitting score of between 2.4 and 2.9 is used, preferably between 2.4 and 2.7, between 2.4 and 2.5. In certain embodiments, a variable fitting score threshold is used depending on the molecular weight of candidate compounds. In further embodiments, candidate compounds of 301 Da to 330 Da have a fitting score threshold of 2.4, candidate compounds of 331 Da to 380 Da a fitting score threshold of 2.5, candidate compounds of 381 Da to 420 Da a fitting score threshold of 2.7, candidate compounds of 421 Da to 490 Da a fitting score threshold of 2.9, and candidate compounds of 491 Da to 540 Da a fitting score threshold of 3.0. When the degree of fit is a relative value, this degree of fit may be expressed relative to a reference compound known to modulate the activity of the Rel protein. In such embodiments, a candidate compound is considered a bona fide modulator of Rel when the degree of fit is at least 50%, preferably at least 60%, at least 70%, at least 80%, at least 85%, at least 90%, most preferably at least 95% to a reference compound known to modulate the activity of Rel. It is evident that a direct comparison between the degrees of fit of multiple ligands may be derived from this initial score. Numerous scoring functions or mechanisms have been described in the art (inter alia in Fu and Zhang, An overview of scoring functions used for protein-ligand interactions in molecular docking, Interdisciplinary Science: Computational Life Sciences, 2019), and it is evident that different such scoring functions are suitable for generating a degree of fit between a candidate compound and the Rel protein. For example, when using the AMBER scoring function (Wang et al., Development and testing of a general amber force field, Journal of Computational Chemistry, 2004), a candidate compound is considered to be a candidate bona fide modulator when a docking score threshold is met. In certain embodiments a docking score threshold of −8.9 kcal/mol is used. In certain embodiments a docking score threshold of between −8.9 kcal/mol and −10.5 kcal/mol is used. In further embodiments a docking score threshold of between −9.4 kcal/mol and −10.5 kcal/mol is used. In yet further embodiments a docking score threshold of between −9.7 kcal and −10.5 kcal/mol is used. In alternative further embodiments a docking score threshold of between −8.9 kcal/mol and −10.3 kcal/mol is used. In further embodiments a docking score threshold of between −8.9 kcal/mol and −9.7 kcal/mol is used. In further further embodiments a docking score threshold of between −8.9 kcal/mol and −9.4 kcal/mol is used. In alternative embodiments, a docking score threshold of −10.5 kcal/mol was used. In yet alternative embodiments, a variable docking score threshold was used, preferably based on the molecular weight of the candidate compounds. In further embodiments, compounds with a molecular weight of 301 Da to 330 Da are assigned a docking score threshold of −8.9 kcal/mol, compounds with a molecular weight of 331 Da to 380 Da are assigned a docking score of −9.4 kcal/mol, compounds with a molecular weight of 381 Da to 420 Da are assigned a docking score of −9.7 kcal/mol, compounds with a molecular weight of 421 Da to 490 Da are assigned a docking score of −10.3 kcal/mol, and compounds with a molecular weight of 491 to 540 Da are assigned a docking score threshold of −10.5 kcal/mol.

In certain embodiments, the method further comprises assessing interactions of said candidate compound to one or more amino acid residues of a region on the surface of the protein defined by amino acid residues: Arg43, Ser45, His156, Thr153, Met157, Asn150, Leu154, Lys161, Arg147, Lys143, Glu168, and 11e165 of the Rel amino acid sequence as defined in SEQ ID NO: 1, wherein an interaction indicates the candidate compound is a modulator of Rel hydrolase activity, or of Rel hydrolase and synthetase activity. In further embodiments, the method comprises assessing whether the candidate compound interacts with at least 2, at least 3, at least 4, at least 5, at least 6, at least 7, at least 8, at least 9, at least 10, at least 11, or all amino acid residues of the group consisting of Arg43, Ser45, His156, Thr153, Met157, Asn150, Leu154, Lys161, Arg147, Lys143, Glu168, and 11e165 of the Rel amino acid sequence as defined in SEQ ID NO: 1. In certain embodiments, the candidate compound is an inhibitor of Rel hydrolase activity.

The term “region on the surface of the protein” as used herein intends to refer to a surface patch that defines a binding site which involves the residues that are listed with respect to said region.

In certain embodiments, the method further comprises assessing interactions of said candidate compound to one or more amino acid residues of a region on the surface of the protein defined by amino acid residues: Asn327, Tyr329, Lys325, His333, Arg277, Arg349, Gln347, Glu345, Asp272, Arg316, Lys251, Arg249, Ala275, Arg355, Ser255, and Lys186 of the Rel amino acid sequence as defined in SEQ ID NO: 1, wherein an interaction indicates the candidate compound is a modulator of Rel synthetase activity or of Rel synthetase and hydrolase activity. In certain embodiments, the method comprises assessing whether the candidate compound interacts with at least 2, at least 3, at least 4, at least 5, at least 6, at least 7, at least 8, at least 9, at least 10, at least 11, at least 12, at least 13, at least 14, at least 15, or all amino acid residues of the group consisting of Asn327, Tyr329, Lys325, His333, Arg277, Arg349, Gln347, Glu345, Asp272, Arg316, Lys251, Arg249, Ala275, Arg355, Ser255, and Lys186 of the Rel amino acid sequence as defined in SEQ ID NO: 1. In certain embodiments, the method further comprises a step to assess whether a candidate compound is an inhibitor of Rel synthetase activity based on interaction with a specific amino acid residue or a specific subset of amino residues of the group described above. In alternative embodiments, the method further comprises a step to assess whether a candidate compound is an inhibitor or Rel synthetase and hydrolase activity based on interaction with a specific amino acid residue or a specific subset of amino residues of the group described above.

In certain embodiments, the method further comprises assessing interactions of said candidate compound to one or more amino acid residues of a region on the surface of the protein defined by amino acid residues: Lys164, Asp200, Tyr201, Arg204, Tyr211, Lys212, His219, Arg221, Arg222, Arg225 of the Rel amino acid sequence as defined in SEQ ID NO: 1, wherein an interaction indicates the candidate compound is an allosteric compound or an effector of the Rel synthetase and/or hydrolase activity. In certain embodiments, the method comprises assessing whether the candidate allosteric compound interacts with at least 2, at least 3, at least 4, at least 5, at least 6, at least 7, at least 8, at least 9, or all amino acid residues of the group consisting of: Lys164, Asp200, Tyr201, Arg204, Tyr211, Lys212, His219, Arg221, Arg222, Arg225 of the Rel amino acid sequence as defined in SEQ ID NO: 1. In certain embodiments, the candidate allosteric compound is an allosteric hydrolase and/or synthetase inhibitor. In alternative embodiments, the candidate allosteric compound is an allosteric hydrolase and/or synthetase activator. In certain embodiments, the allosteric compound is a Rel hydrolase inhibitor and a Rel synthetase activator. In certain embodiments, the allosteric compound is a Rel hydrolase activator and a Rel synthetase inhibitor. In certain embodiments, the candidate allosteric compound inhibits the synthetase and/or hydrolase activity by at least 50%, preferably at least 60%, preferably at least 75%, more preferably at least 90%, most preferably at least 95% compared to the Rel synthetase activity and/or hydrolase activity in absence of said allosteric compound. In certain embodiments, the method further comprises comparing the conformational state of Rel with or without said candidate compound binds to the allosteric site of Rel, wherein a change in conformational state is indicative for the candidate compound to be a bona fide effector of the Rel hydrolase and/or synthetase activity, preferably wherein the conformational state of Rel without candidate binding is the conformational state characterized by the atomic coordinates of Table 4.

An “allosteric site” of an enzyme as used herein refers to a site not part of an active site of said enzyme discussed, thus a site other than the enzyme's active site(s). It is said that the regulatory site of an allosteric protein is physically distinct from its active (catalytic or enzymatic) site (Kirschner, Allosteric regulation of enzyme activity; an introduction to the molecular basis of and the experimental approaches to the problem, Current topics in microbiology and immunology, 1968). “Allosteric modulators” or “allosteric modulators” in the context of the invention are therefore modulators that bind to a site different to the enzyme's active site(s) but nonetheless have an effect on the enzymatic activity of said enzyme. Allosteric modulators that enhance the activity of an enzyme are referred to as allosteric activators. The latter can initiate and/or maintain a hyperactive enzyme. In contrast, modulators that decrease the enzymatic activity are called allosteric inhibitors. In the context of the current invention, allosteric modulators may be allosteric activators for a first enzymatic (hydrolase or synthetase) activity and optionally also be an allosteric inhibitor for a second enzymatic (hydrolase or synthetase) activity of the target protein. Since allosteric regulation is often induced by an effect of the allosteric modulator on the conformation of the target enzyme or affected enzymatic domain, it is therefore said that allosteric modulators may induce a conformational change on the protein they bind to.

In certain embodiments, the method further comprises determining a score of a candidate compound to modulate Rel hydrolase and/or Rel synthetase activity based on the number of interactions with said amino acid residues. In certain embodiments, the score is directly proportional to the amount of interactions with said residues. A skilled person understands that in these embodiments the score for Rel hydrolase modulators is dependent on the amount of interactions with amino acid residues of the group consisting of Arg43, Ser45, His156, Thr153, Met157, Asn150, Leu154, Lys161, Arg147, Lys143, Glu168, and Ile165 of the Rel amino acid sequence as defined in SEQ ID NO: 1, the score for Rel hydrolase modulators is dependent on the amount of interactions with amino acid residues of the group consisting of Asn327, Tyr329, Lys325, His333, Arg277, Arg349, Gln347, Glu345, Asp272, Arg316, Lys251, Arg249, Ala275, Arg355, Ser255, and Lys186 of the Rel amino acid sequence as defined in SEQ ID NO: 1, and the score for allosteric Rel synthetase and/or Rel hydrolase modulators is dependent on the amount of interactions with amino acid residues of the group consisting of Lys164, Asp200, Tyr201, Arg204, Tyr211, Lys212, His219, Arg221, Arg222, Arg225 of the Rel amino acid sequence as defined in SEQ ID NO: 1. It is evident that a single candidate compound may be characterized by two separate scores indicative for Rel hydrolase and Rel synthetase modulation respectively when compared to the hydrolase and synthetase activity of an identical Rel protein in absence of any hydrolase and/or synthetase modulator. The score may be expressed as an absolute value and/or as a relative value compared to one or more reference Rel modulator molecules. In an illustrative embodiment, the score may be a positive integer that is a sum of the number of interactions between the amino acid residues described herein and the candidate compound. In an alternative illustrative embodiment, the score may be a percentage, wherein 0% indicates no interaction(s) between the candidate compound and the Rel protein, and 100% indicates an interaction with each of the amino acid residues described herein that are indicated to form, or be part of, the relevant portion of the Rel surface region as defined herein. It is evident that a candidate compound with a higher score, said score being linearly correlated to the amount of interactions, indicates a higher likelihood of a candidate compound to be a strong modulator (e.g. inhibitor) of the Rel protein when compared to a candidate compound with a lower score.

In certain embodiments, the method further comprises comparing the conformational state of Rel before and after said candidate compound binds to Rel, wherein a change in conformational state is indicative for the candidate compound to be a bona fide modulator of Rel hydrolase and/or Rel synthetase activity, preferably wherein the conformational state of Rel before candidate binding is the resting conformational state characterized by the atomic coordinates of Table 1. In certain embodiments, the method comprises detection of any atomic coordinates that are different after binding of the candidate Rel modulator from the atomic coordinates characterizing the resting conformational state of Rel shown in Table 1. By a conformational change as used herein it is intended a structural change, or structural transition, in the three dimensional structure of the Rel before and after binding of the candidate compound to Rel. A conformational change can be any transition from the following Rel conformations: open conformation, closed conformation, intermediate conformation (indicative for a structurally folded Rel protein that is distinct from the open and closed conformation), and an (partially) unfolded Rel conformation.

In certain embodiments, the method further comprises comparing the conformational state of Rel with or without said candidate compound binds to Rel, wherein a change in conformational state is indicative for the candidate compound to be a bona fide modulator of Rel hydrolase or Rel hydrolase and synthetase activity, preferably wherein the conformational state of Rel without candidate binding is the (p)ppGpp bound conformational state characterized by the atomic coordinates of Table 3. In further embodiments, the candidate compound is considered a Rel hydrolase inhibitor by the methods described herein when upon binding with one or more of said Rel amino acid residues, Rel is stabilized in an open state. In further embodiments, the candidate compound (completely) inhibits Rel hydrolase activity and partially inhibits Rel synthetase activity when Rel is stabilized in an open state. In certain embodiments, the candidate inhibitor reduces Rel hydrolase activity by at least 50%, preferably at least 60%, preferably at least 75%, preferably at least 90%, more preferably at least 95%, most preferably at least 99% compared to the Rel hydrolase activity in absence of said inhibitor.

In certain embodiments, the method further comprises comparing the conformational state of Rel with or without said candidate compound binds to Rel, wherein a change in conformational state is indicative for the candidate compound to be a bona fide modulator of Rel synthetase or Rel synthetase and hydrolase activity, preferably wherein the conformational state of Rel without candidate binding is the AMP-G4P bound conformational state characterized by the atomic coordinates of Table 2. In further embodiments, the candidate compound is considered a Rel synthetase inhibitor when upon binding with one or more of said Rel amino acid residues, Rel is stabilized in a closed state. In further embodiments, the candidate compound (completely) inhibits Rel synthetase activity and partially inhibits Rel hydrolase activity when Rel is stabilized in an open state. In certain embodiments, the candidate inhibitor reduces Rel synthetase activity by at least 50%, preferably at least 60%, preferably at least 75%, preferably at least 90%, more preferably at least 95%, most preferably at least 99% compared to the Rel hydrolase activity in absence of said inhibitor.

In certain embodiments, the method further comprises testing of the ability of the candidate compounds for modulating Rel synthetase and/or Rel hydrolase activity. In certain embodiments, the method comprises in vitro and/or in vivo testing of the ability of the candidate compounds for Rel modulation Rel synthetase and/or Rel hydrolase activity. In certain embodiments, the testing of the candidate compounds involves testing of said compound in competition with one or more natural Rel substrates including but not limited to (p)ppGpp and/or AMP-G4P.

By means of an illustrative example, in vitro testing of the hydrolase activity of Rel in presence of an candidate Rel synthetase-modulating compound can comprise contacting said candidate compound with recombinant Rel protein and measuring removal of the 3′ pyrophosphate moiety from (p)ppGpp (i.e. monitoring the hydrolysis reaction mediated by Rel). In vitro synthetase activity testing can be performed in a similar assay, whereby synthesis of (p)ppGpp can be monitored (i.e. transfer of the pyrophosphate group of ATP onto the 3′ of GDP or GTP). Similar experimental conditions can be devised for in vivo activity testing. Methods for assessing a plethora of different enzymatic activities are known in the art (Ou et al., Methods of measuring enzyme activity ex vivo and in vivo, Annual review of analytical chemistry, 2018).

In certain embodiments, the methods described herein are computer-implemented methods. In further embodiments, the computer comprising an inputting device, a processor, a user interface, and an outputting device, wherein said method comprises the steps of:

a) generating a three-dimensional structure of said atomic coordinates, or said subset thereof;

b) fitting the structure of step a) with the structure of a candidate compound by computational modeling;

c) selecting a ligand that possesses energetically favorable interactions with the structure of step a).

In certain embodiments, the method further comprises selection of ligands that possess multiple energetically favorable interactions with said three-dimensional structure in favor of ligands that possess one energetically favorable interaction with said three-dimensional structure. In certain embodiments, the three-dimensional structure is generated using the atomic coordinates from at least one list of atomic coordinates of Table 2, 3, or 4. In alternative embodiments, the three-dimensional structure is generated using a subset of atomic coordinates from Table 2, 3, or 4. In further embodiments, the three-dimensional structure is generated using a subset of atomic coordinates that are unique for Table 2, 3, or 4. By the term “energetically favorable interaction” as used herein is envisaged any interaction with interaction energies <0 kJ/mol. Alternatively an energetically favorable interaction may be expressed as an interaction having a negative Gibbs free energy (ΔG) value. Since a protein-ligand association extent is correlated to the magnitude of a negative ΔG, ΔG can be regarded as determinant for the stability of the protein-ligand complex under investigation, or, alternatively, the binding affinity of a ligand to a given acceptor, in the context of the current specification the RSH enzyme Rel. Free energy is a function of the states of a system and, as thus, ΔG values are defined by the initial and final thermodynamic state, regardless of any intermediates states. The concept of energetically favorable interactions is known to a person skilled in the art (Du et al., Insights into Protein-Ligand Interactions: Mechanisms, Models, and Methods, International journal of molecular sciences, 2016).

In certain embodiments, the method comprises superimposing the generated three-dimensional structure with the structure of the candidate compound. In further embodiments, the method comprises selecting from a collection of distinct structure-candidate compound superimposed orientations a most favorable orientation of said structure with said candidate compound. Hence, in certain embodiments, the method comprises docking modeling or molecular docking. In certain embodiments, the method comprises a computer-implemented step of proposing candidate structure modifications to further increasing the number of favorable interactions with the generated three-dimensional structure. In yet further embodiments, the method comprises ranking an obtained collection of candidate compounds based on the number of favorable interactions they engage in with the generated three-dimensional structure, wherein candidate compounds with a higher number of favorable interactions are ranked higher than candidate compounds with fewer favorable interactions.

The terms “docking modeling” and “molecular docking” are indicative for one or more quantitative and/or qualitative analyses of a molecular structure based on structural information and interaction models. Modeling may refer to any one of numeric-based molecular dynamic models, interactive computer graphic models, energy minimization models, distance geometry, molecular mechanics models, or any structure-based constraints model. These illustrative molecular modeling approaches may be employed to the atomic coordinates or a subset of atomic coordinates as described herein in any one of Tables 1, 2, 3, or 4 to obtain a range of three-dimensional models and to investigate the structure of any binding sites, such as the binding sites of candidate Rel modulators. Modeling methods and tools have been developed to design or select chemical molecules that have a complementarity to particular target regions, in the context of the invention a particular target region of Rel. In certain embodiments, the chemical molecule, i.e. the candidate compound has a stereochemical complementarity to said target regions. Stereochemical complementarity refers to a scenario wherein there are a number of energetically favorable contacts between the candidate compound and (the target region of) Rel. A skilled person appreciates that if a certain number of energetically favorable interactions are sufficient to modulate Rel activity, and that it is thereby not a precondition that all the key amino acid residues as described herein are engaged in an energetically favorable interaction. Non-limiting examples of software programs suitable for conducting molecular docking analysis have been described in detail in the art (Pagadala et al., Software for molecular docking: a review, Biophysical Reviews, 2017).

Any computer system or any computer-implemented method relying on a computer system described herein may further comprise means for machine learning of said device to predict candidate Rel modulators and/or score candidate Rel modulators based on input of a reference set of candidate compounds by a user, or based on date generated from earlier fitting and/or selection steps of candidate modulators. The combination of machine learning models for in silico screening and prediction of enzyme binding molecules or modulators is known in the art, and therefore also envisaged by the current invention (Li, et al., Machine learning models combined with virtual screening and molecular docking to predict human Topoisomerase I inhibitors, Molecules, 2019). Non-limiting examples of machine learning models, i.e. machine learning algorithms include Linear regression, logistic regression, decision trees, support vector machines, naive Bayes, k-nearest neighbors (kNN), k-means, random forest, dimensionality reduction algorithms, and gradient boosting algorithms such as gradient boosting machine (GBM), XGBoost, LightGBM, and CatBoost.

In certain embodiments, the method comprises selecting a candidate compound that can bind to at least 1 amino acid residue, preferably more than 1 amino acid residue of the generated three-dimensional structure without steric interference. The terms “steric interference”, “steric hindrance”, and “steric effects” are known to a person skilled in the art. Steric interference or alternatively referred to as steric hindrance is a consequence of a steric effect, and indicates the slowing of chemical reactions due to steric bulk.

Further aspects herein relate to an in vitro method for identifying a compound which modulates Rel hydrolase and/or synthetase activity comprising the steps of:

a) providing a candidate compound;

b) providing the Rel polypeptide;

c) contacting said candidate compound with said Rel polypeptide;

d) determining the hydrolase and/or synthetase activity of Rel in the presence and absence of said candidate compound; and

e) identifying said candidate compound as a compound which modulates Rel hydrolase and/or synthetase activity if a change in activity is detected.

An illustrative method to assess hydrolase and/or synthetase activity is described above. In certain embodiments, the method comprises further selecting additional candidate compounds based on common structural features from a database. In certain embodiments, recombinant Rel protein is used in the methods described herein. Means and method to produce and purify recombinant protein have been described in detail in the art (inter alia in Grasslund et al., Protein production and purification, Nature methods, 2011). In certain embodiments, the complete Rel amino acid sequence is provided (i.e. SEQ ID NO: 1) or an amino acid sequence with at least 70%, preferably at least 80% sequence identity to SEQ ID NO: 1, while in alternative embodiments a functional fragment of Rel is provided, for example the hydrolase domain or the synthetase domain. In certain embodiments, the method may express a difference in hydrolase and/or synthetase activity or Rel in presence of absence of a candidate compound by a quantitative indication, such as a ratio. In certain embodiments, the method further comprises immobilization of the Rel protein or the candidate compound on a solid surface. In further embodiments, the method comprises a step of washing away excess Rel protein or excess candidate compound prior to determining the hydrolase and/or synthetase activity. In certain embodiments, the method comprises detecting a change in hydrolase and/or synthetase activity by colorimetry or spectrophotometry. In certain embodiments, a change of activity is considered as an increase of hydrolase and/or synthetase activity of the Rel protein by at least 10%, preferably 25%, preferably 50%, preferably 75%, preferably 100% in presence of said candidate compound when compared to the respective hydrolase and/or synthetase activity when the enzymatic activity of said Rel protein is assessed in absence of any (candidate) compound. In certain embodiments, a change of activity is considered as a decrease of hydrolase and/or synthetase activity of the Rel protein by at least 10%, preferably 25%, preferably 50%, preferably 75%, preferably 100% in presence of said candidate compound when compared to the respective hydrolase and/or synthetase when the enzymatic activity of said Rel protein is assessed in absence of any (candidate) compound. In certain embodiments, the method identifies candidate compounds capable of inhibiting the hydrolase and/or synthetase activity. In alternative embodiments, the method identifies candidate compounds capable of stimulating the hydrolase and/or synthetase activity.

Using the crystal structure as defined herein, the inventors have identified a number of candidate compounds that fit within said model and have subsequently confirmed their modulatory effect on the Rel enzyme activity.

Thus, a further aspect of the invention relates to Rel modulators obtained by any of the methods described herein. In certain embodiments, the present invention relates to a modulator of Rel hydrolase and/or synthetase activity obtained by the methods as defined herein. In certain embodiments, the present invention relates to an inhibitor of Rel hydrolase and/or synthetase activity obtained by the methods as defined herein. In certain embodiments, the present invention relates to an activator of Rel hydrolase and/or synthetase activity obtained by the methods as defined herein. In certain embodiments, the present invention relates to an effector of Rel hydrolase and/or synthetase activity obtained by the methods as defined herein.

The present invention thus also relates to modulators of Rel hydrolase and/or synthetase activity. In an embodiment, a compound as identified herein as “modulator of Rel hydrolase and/or synthetase activity” is a compound having a general formula selected from the group comprising formula (I), formula (II), formula (III) and formula (IV) as defined herein. The term “a compound of formula (I)”, or “a compound of formula (II)”, or “a compound of formula (III)”, or “a compound of formula (IV)”, as described herein, intends to also encompass an isomer, preferably a stereo-isomer or a tautomer, a solvate, a salt, preferably a pharmaceutically acceptable salt, or a prodrug of said (respective) compound, preferably a pharmaceutically acceptable salt, solvate, hydrate, polymorph, tautomer, stereoisomer, or prodrug of said (respective) compound.

In an embodiment, a compound as identified herein as modulator of Rel hydrolase and/or synthetase activity is
(i) a compound of formula (I), or an isomer, preferably a stereo-isomer or a tautomer, a solvate, a salt, preferably a pharmaceutically acceptable salt, or a prodrug thereof,

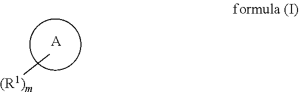

wherein m is an integer selected from 1, 2, 3, 4, 5, 6, or 7;
wherein each R¹ is independently selected from halogen, ═O, nitro, or a group comprising hydroxyl, —SH, —NH₂, C(O)OH, heterocyclyl, heteroaryl, alkyl, haloalkyl, cycloalkyl, cycloalkenyl, hydroxycarbonylalkyl, hydroxycarbonylalkenyl, alkenyl, aryl, heteroalkyl, heteroalkenyl, alkyloxy, arylalkyl, arylalkenyl-, aryl-heteroalkyl-, aryl-heteroalkenyl-, aryl-heteroaryl-; aryl-heterocyclyl-, heterocyclyl-alkyl-, heterocyclyl-alkenyl-, heterocyclyl-heteroalkyl-, heterocyclyl-heteroalkenyl-, heteroaryl-alkyl-, heteroaryl-alkenyl-, heteroaryl-heteroalkyl-, heteroaryl-heteroalkenyl-, alkyloxy-, haloalkoxy-, alkenyloxy-, aryloxy-; heteroaryloxy-, heterocylyloxy-, alkylthio-, alkenylthio-, arylthio-, heteroarylthio-, heterocyclylthio-, aryl-alkyloxy-, heteroaryl-alkyloxy-, heterocyclyl-alkyloxy-, aryl-alkylthio-, heteroaryl-alkylthio-, heterocyclyl-alkylthio-, alkyl-SO2-, alkenyl-SO2-, heteroalkyl-SO2-, heteroalkenyl-SO2-, aryl-SO2-, heteroaryl-SO2-, heterocyclyl-SO2-, Heteroaryl-heteroalkyl-heteroaryl-, Heteroaryl-heteroalkenyl-heteroaryl-, heteroaryl-alkyl-heteroaryl-, Heteroaryl-alkenyl-heteroaryl-, Aryl-heteroaryl-alkyl-, aryl-heteroaryl-heteroalkyl-, aryl-heteroaryl-alkenyl-, aryl-heteroaryl-heteroalkenyl-, Alkyloxy-aryl-alkyl-, Alkyloxy-heteroaryl-alkyl-, Alkyloxy-aryl-alkenyl-, Alkyloxy-heteroaryl-alkenyl-, Alkyloxy-heterocyclyl-alkyl-, Alkyloxy-heterocyclyl-alkenyl-, Alkyloxy-heterocyclyl-heteroalkyl-, Alkyloxy-heterocyclyl-heteroalkenyl-, aryl-alkenyl-heteroaryl-heteroalkyl, aryl-alkyl-heterocyclyl-heteroalkyl, Aryl-heteroalkyl-heteroaryl-, aryl-heteroalkenyl-heteroaryl-, aryl-heteroalkyl-heterocyclyl-, aryl-heteroaryl-heterocyclyl-; Aryl-heteroalkyl-heteroaryl-alkyl-, alkenyl-aryl-heteroalkenyl, Aryl-alkyl-heterocyclyl-SO2-, aryl-alkenyl-heterocyclyl-SO2-, heteroaryl-alkyl-heterocyclyl-SO2-, heteroaryl-alkenyl-heterocyclyl-SO2-, Aryl-heteroalkenyl-heteroaryl-alkyl-, alkenyl-aryl-heteroalkenyl-, Aryl-Alkyl-heterocyclyl-alkyl-, Aryl-Alkyl-heterocyclyl-alkenyl-, Aryl-Alkyl-heterocyclyl-heteroalkyl-, Aryl-Alkyl-heterocyclyl-heteroalkenyl-, Aryl-alkenyl-heterocyclyl-alkyloxy-, Aryl-alkyl-heterocyclyl-alkyloxy-, Aryl-alkenyl-heteroaryl-alkyloxy-, Aryl-alkyl-heteroaryl-alkyloxy-, aryl-imino-, heteroalkyl-aryl-imino-, alkenyl-aryl-imino-; and wherein each of said groups can be unsubstituted or substituted with one or more substituents each independently selected from the group comprising halogen, nitro, oxo, alkyloxy, —C(O)OH, —NH₂, hydroxycarbonylalkyl, hydroxycarbonylalkenyl, hydroxyl, alkyl, alkenyl, haloalkyl, haloalkenyl, heteroalkyl, heteroalkenyl, ═S, —SH, aryl, nitroaryl-, heteroaryl, heterocyclyl; aryl-alkyl-; aryl-alkenyl-; arylheteroalkyl-; arylheteroalkenyl-; heterocyclyl-alkyl-imino, aryl-imino-, heteroalkyl-aryl-imino-; and
wherein cycle A is selected from the group represented by formula (Ia);

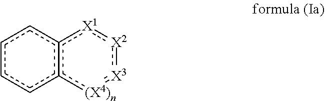

wherein the dotted line represents an optional double bond;
wherein n is an integer selected from 0 or 1;
wherein each of X¹, X², X³, and X⁴ is independently selected from the group comprising N, NH, S, O, C═O, C═S, CH, C(Z¹)₂, and N(Z²), and
   wherein each Z¹ is independently selected from selected from the group comprising hydrogen, halogen, nitro, hydroxyl, —SH, —NH₂, —C(O)OH, alkyl, alkenyl, hydroxycarbonylalkyl, hydroxycarbonylalkenyl, heteroalkyl, heteroalkenyl, aryl, heteroaryl, heterocyclyl, alkyloxy, alkylthio, arylalkyl-, aryl-alkenyl-, aryl-heteroalkyl-, aryl-heteroalkenyl-, heterocyclyl-heteroalkyl, heterocyclyl-heteroalkenyl-, heteroaryl-heteroalkyl-, heteroaryl-heteroalkenyl-, Heteroaryl-heteroalkyl-heteroaryl-, Heteroaryl-heteroalkenyl-heteroaryl-, Aryl-heteroaryl-alkyl-, Aryl-heteroaryl-heteroalkyl-, Aryl-heteroaryl-alkenyl, Aryl-heteroaryl-heteroalkenyl, Alkyloxy-aryl-alkyl-, and Alkyloxy-aryl-alkenyl-; and
   wherein each Z² is independently selected from the group comprising alkyl, alkenyl, hydroxycarbonylalkyl, hydroxycarbonylalkenyl, heteroalkyl, heteroalkenyl, aryl, heteroaryl, heterocyclyl, aryl-heteroalkyl-, aryl-heteroalkenyl-, heterocyclyl-heteroalkyl, heterocyclyl-heteroalkenyl-, heteroaryl-heteroalkyl-, heteroaryl-heteroalkenyl-, Heteroaryl-heteroalkyl-heteroaryl-, Heteroaryl-heteroalkenyl-heteroaryl-, Aryl-heteroaryl-alkyl-, Aryl-heteroaryl-heteroalkyl-, Aryl-heteroaryl-alkenyl, Aryl-heteroaryl-heteroalkenyl, Alkyloxy-aryl-alkyl-, and Alkyloxy-aryl-alkenyl-; or
wherein when X² and X³ are each independently selected from C(Z¹)₂ or N(Z²) as defined above, two Z¹, or Z¹ together with Z² together with the atom to which they are attached form a ring selected from the group comprising heterocyclyl, C₃₋₁₈cycloalkyl, cycloalkenyl, aryl, and heteroaryl.
or
(ii) a compound of formula (II), or an isomer, preferably a stereo-isomer or a tautomer, a solvate, a salt, preferably a pharmaceutically acceptable salt, or a prodrug thereof,

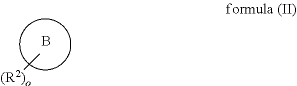

wherein o is an integer selected from 1, 2, 3, 4, 5, 6, or 7; and preferably selected from 1, 2, 3, 4, or 5, and preferably selected from 1, 2, 3, or 4;
wherein each R² is independently selected from halogen, ═S, ═O, nitro, or a group comprising hydroxyl, —SH, —NH₂, —C(O)OH, alkyl, alkenyl, haloalkyl, cycloalkyl, cycloalkenyl, heteroalkyl, heteroalkenyl, aryl, heteroaryl, heterocyclyl, arylalkyl-, arylalkenyl-, aryl-heteroalkyl-, aryl-heteroalkenyl-, aryl-heteroaryl-; aryl-heterocyclyl-, heterocyclyl-alkyl-; heterocyclyl-alkenyl-, heterocyclyl-heteroalkyl-, heterocyclyl-heteroalkenyl-, heteroaryl-alkyl-, heteroaryl-alkenyl-, heteroaryl-heteroalkyl-, heteroaryl-heteroalkenyl-, alkyloxy, haloalkoxy, alkenyloxy, aryloxy; heteroaryloxy, heterocylyloxy, alkylthio, alkenylthio, arylthio, heteroarylthio, heterocyclylthio, aryl-alkyloxy-, heteroaryl-alkyloxy-, heterocyclyl-alkyloxy-, aryl-alkylthio-, heteroaryl-alkylthio-, heterocyclyl-alkylthio-, hydroxycarbonylalkyl-, hydroxycarbonylalkenyl-, alkyl-SO2-, alkenyl-SO2-, heteroalkyl-SO2-, heteroalkenyl-SO2-, aryl-SO2-, heteroaryl-SO2-, heterocyclyl-SO2-, heteroaryl-alkyl-SO2-; heteroaryl-alkenyl-SO2-, heteroaryl-heteroalkyl-SO2-, heteroaryl-heteroalkenyl-SO2- and heteroaryl-NH—SO2-,
   and wherein each of said groups can be unsubstituted or substituted with one or more substituents each independently selected from the group comprising halogen, nitro, oxo (═O), alkyloxy, —C(O)OH, —NH₂, hydroxycarbonylalkyl, hydroxycarbonylalkenyl, hydroxyl, alkyl, alkenyl, heteroalkyl, heteroalkenyl ═S, —SH, aryl, heteroaryl, heterocyclyl.
wherein cycle B is selected from the group represented by formula (IIa);

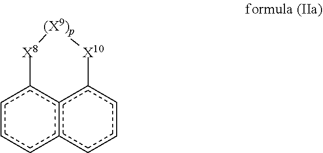

wherein the dotted line represents an optional double bond;
wherein p is an integer selected from 0 or 1;
wherein each of X⁸, X⁹, and X¹⁰ is independently selected from NH, N—OH, S, O, C═O, C═S, C(Z³)₂ and N(Z⁴), and wherein each Z³ is independently selected from the group comprising hydrogen, halogen, nitro, hydroxyl, —SH, —NH₂, —C(O)OH, alkyl, alkenyl, hydroxycarbonylalkyl, hydroxycarbonylalkenyl, heteroalkyl, heteroalkenyl, aryl, heteroaryl, heterocyclyl, and alkyloxy, and wherein each Z⁴ is independently selected from the group comprising alkyl, alkenyl, hydroxycarbonylalkyl, hydroxycarbonylalkenyl, heteroalkyl, heteroalkenyl, aryl, heteroaryl, heterocyclyl, and alkyloxy;
   and preferably with the proviso that when p is 1 and X⁹ is N—OH, then X⁸ and X¹⁰ are C═O, and/or
   preferably with the proviso that when p is 0 and X⁸ is NH, then X¹⁰ is C═O, or when p is 0 and X⁸ is C═O, then X¹⁰ is NH.
or
(iii) a compound of formula (III), or an isomer, preferably a stereo-isomer or a tautomer, a solvate, a salt, preferably a pharmaceutically acceptable salt, or a prodrug thereof,

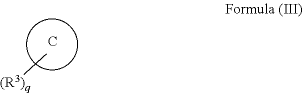

wherein q is an integer selected from 1, 2, 3, 4, 5, or 6; and
wherein each R³ is independently selected from halogen, ═S, ═O, nitro, or a group comprising hydroxyl, —SH, —NH₂, —C(O)OH, alkyl, alkenyl, haloalkyl, hydroxycarbonylalkyl, hydroxycarbonylalkenyl, cycloalkyl, cycloalkenyl, heteroalkyl, heteroalkenyl, aryl, heteroaryl, heterocyclyl, alkyloxy, alkylthio, heterocyclyl-alkyl-, heterocyclyl-heteroalkyl-, heterocyclyl-heteroaryl, aryl-alkyl-, arylalkenyl-, aryl-heteroalkyl-; aryl-heteroalkenyl-, aryl-heteroaryl-; aryl-heterocyclyl-, heterocyclyl-alkyl-; heterocyclyl-alkenyl-, heterocyclyl-heteroalkenyl-, heteroarylalkyl-, heteroarylalkenyl-, heteroaryl-heteroalkyl-, heteroaryl-heteroalkenyl-, alkyloxy-, alkenyloxy-, aryloxy-; heteroaryloxy-, heterocylyloxy-, alkylthio-, alkenylthio-, arylthio-, heteroarylthio-, heterocyclylthio-, aryl-alkyloxy-, heteroaryl-alkyloxy-, heterocyclyl-alkyloxy-, aryl-alkylthio-, heteroaryl-alkylthio-, heterocyclyl-alkylthio-, alkyl-SO2-, alkenyl-SO2-, heteroalkyl-SO2-, heteroalkenyl-SO2-, aryl-SO2-, heteroaryl-SO2-, heterocyclyl-SO2-, heteroaryl-heteroalkyl-heteroaryl-, Heteroaryl-heteroalkenyl-heteroaryl-, heteroaryl-alkyl-heteroaryl-, Heteroaryl-alkenyl-heteroaryl-, Aryl-heteroaryl-alkyl-, aryl-heteroaryl-alkenyl-, aryl-heteroaryl-heteroalkyl-, aryl-heteroaryl-heteroalkenyl-, aryl-alkenyl-, Alkyloxy-aryl-alkyl-, Alkyloxy-heteroaryl-alkyl-, Alkyloxy-heteroaryl-alkenyl-, Alkyloxy-heterocyclyl-alkyl-, Alkyloxy-heterocyclyl-alkenyl-, Alkyloxy-heterocyclyl-heteroalkyl-, Alkyloxy-heterocyclyl-heteroalkenyl-, Aryl-heteroalkyl-heteroaryl-, aryl-heteroalkenyl-heteroaryl-, Aryl-heteroalkyl-heteroaryl-alkyl-, Aryl-heteroalkenyl-heteroaryl-alkyl-, aryl-heteroalkyl-heterocyclyl-, aryl-heteroaryl-heterocyclyl-; Aryl-alkyl-heterocyclyl Aryl-alkenyl-heterocyclyl, alkenyl-aryl-heteroalkenyl-, Aryl-Alkyl-heterocyclyl-alkyl-, Aryl-Alkyl-heterocyclyl-alkenyl-, Aryl-Alkyl-heterocyclyl-heteroalkyl-, Aryl-Alkyl-heterocyclyl-heteroalkenyl-, Aryl-alkenyl-heterocyclyl-alkyloxy-, Aryl-alkyl-heterocyclyl-alkyloxy-, Aryl-alkenyl-heteroaryl-alkyloxy-, Aryl-alkyl-heteroaryl-alkyloxy-, Aryl-alkyl-heterocyclyl-SO2-, aryl-alkenyl-heterocyclyl-SO2-, heteroaryl-alkyl-heterocyclyl-SO2-, heteroaryl-alkenyl-heterocyclyl-SO2-; aryl-amino, and aryl-NH— and wherein each of said groups can be unsubstituted or substituted with one or more substituents each independently selected from the group comprising halogen, nitro, oxo, alkyloxy, —C(O)OH, —NH₂, hydroxycarbonylalkyl, hydroxycarbonylalkenyl, hydroxyl, alkyl, alkenyl, haloalkyl, haloalkenyl, heteroalkyl, heteroalkenyl ═S, —SH, aryl, heteroaryl, heterocyclyl; aryl-alkyl-; aryl-alkenyl-; arylheteroalkyl-; arylheteroalkenyl-; and heterocyclyl-alkyl-; and
wherein cycle C is selected from the group represented by formula (IIIa);

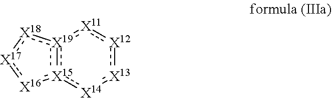

wherein the dotted line represents an optional double bond;
wherein each of X¹⁵ and X¹⁹ is independently selected from N, C, and CH,
wherein each of X¹¹, X¹², X¹³, X¹⁴, X¹⁶, X¹⁷, and X¹⁸ is independently selected from the group comprising N, NH, S, O, C═O, C═S, CH, C(Z⁵)₂ and N(Z⁶), and
   wherein each Z⁵ is independently selected from the group comprising hydrogen, halogen, nitro, hydroxyl, —SH, —NH₂, —C(O)OH, alkyl, heteroalkyl, alkenyl, heteroalkenyl, haloalkyl, aryl, heteroaryl, heterocyclyl, alkyloxy, alkylthio, heterocyclyl-alkyl-, heterocyclyl-alkenyl-, heterocyclyl-heteroalkyl-, heterocyclyl-heteroalkenyl-, aryl-alkyl-, aryl-alkenyl-, aryl-heteroalkyl-; aryl-heteroalkenyl-, heteroaryl-alkyl-, heteroaryl-alkenyl-, heteroaryl-heteroalkyl-, heteroaryl-heteroalkenyl-, heteroaryl-alkyl-heteroaryl-, Heteroaryl-heteroalkyl-heteroaryl-, Aryl-heteroaryl-alkyl-, Aryl-heteroaryl-alkenyl, aryl-heteroaryl-heteroalkyl-, Aryl-heteroaryl-heteroalkenyl, aryl-heteroalkyl-heterocyclyl-, hydroxycarbonylalkyl, and hydroxycarbonylalkenyl; and
   wherein each Z⁶ is independently selected from the group comprising alkyl, alkenyl, haloalkyl, hydroxycarbonylalkyl, hydroxycarbonylalkenyl, heteroalkyl, heteroalkenyl, aryl, heteroaryl, heterocyclyl, alkyloxy, arylalkyl-, arylalkenyl-, arylheteroalkyl-, arylheteroalkenyl-, heteroaryl-alkyl-, heteroaryl-alkenyl-, heterocyclyl-alkyl-, heterocyclyl-alkenyl-, heterocyclyl-heteroalkyl-; heterocyclyl-heteroalkenyl-, heteroaryl-heteroalkyl-, heteroaryl-heteroalkenyl-, heteroaryl-alkyl-heteroaryl-, Heteroaryl-heteroalkyl-heteroaryl-, Aryl-heteroaryl-alkyl-, Aryl-heteroaryl-alkenyl, aryl-heteroaryl-heteroalkyl-, and Aryl-heteroaryl-heteroalkenyl.
or
(iv) a compound of formula (IV) or an isomer, preferably a stereo-isomer or a tautomer, a solvate, a salt, preferably a pharmaceutically acceptable salt, or a prodrug thereof,

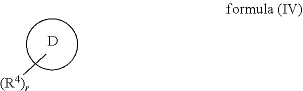

wherein cycle D is selected from the group heteroaryl, aryl, heterocyclyl, and cycloalkyl;
wherein r is an integer selected from 1, 2, 3, 4, 5 or 6; and preferably selected from 1, 2, 3 or 4; and
wherein each R⁴ is independently selected from halogen, nitro, or a group comprising hydroxyl, —NH₂, —C(O)OH, alkyl, alkenyl, haloalkyl, cycloalkyl, cycloalkenyl, heteroalkyl, heteroalkenyl, aryl, heteroaryl, heterocyclyl, arylalkyl-, arylalkenyl-, aryl-heteroalkyl-, aryl-heteroalkenyl-, aryl-heteroaryl-; aryl-heterocyclyl-, alkyl-heteroaryl-, alkenyl-heteroaryl-, heteroalkyl-heteroaryl-, heteroalkyl-heterocyclyl, heteroalkenyl-heteroaryl-, heteroalkenyl-heterocyclyl-, heterocyclyl-alkyl-; heterocyclyl-alkenyl-, heterocyclyl-heteroalkyl-, heterocyclyl-heteroalkenyl-, heterocyclyl-heterocyclyl-, heteroaryl-alkyl-, heteroaryl-alkenyl-, heteroaryl-heteroalkyl-, heteroaryl-heteroalkenyl-, alkyloxy, haloalkoxy, alkenyloxy, aryloxy; heteroaryloxy, heterocylyloxy, alkylthio, alkenylthio, arylthio, heteroarylthio, heterocyclylthio, aryl-alkyloxy-, heteroaryl-alkyloxy-, heterocyclyl-alkyloxy-, aryl-alkylthio-, heteroaryl-alkylthio-, heterocyclyl-alkylthio-, hydroxycarbonylalkyl, hydroxycarbonylalkenyl, alkyl-SO2-, alkenyl-SO2-, heteroalkyl-SO2-, heteroalkenyl-SO2-, aryl-SO2-, heteroaryl-SO2-, heterocyclyl-SO2-, heteroaryl-alkyl-SO2-; heteroaryl-alkenyl-SO2-, heteroaryl-heteroalkyl-SO2-, heteroaryl-heteroalkenyl-SO2-, heteroaryl-heteroalkyl-heteroaryl-, heteroaryl-heteroalkenyl-heteroaryl-, heteroaryl-alkyl-heteroaryl-, heteroaryl-alkenyl-heteroaryl-, Aryl-heteroaryl-alkyl-, aryl-heteroaryl-alkenyl-, aryl-heteroaryl-heteroalkyl-, aryl-heteroaryl-heteroalkenyl-, aryl-heteroalkyl-heteroaryl-; aryl-heteroalkyl-aryl-; aryl-heteroalkyl-heteroaryl-heteroalkyl-; aryl-heteroalkyl-heteroaryl-heteroalkenyl, heteroaryl-heterocylcyl-alkyl, and aryl-NH—, aryl-NH-heteroaryl-heteroalkenyl-, nitroaryl-, nitroaryl-NH—, nitroaryl-NH-heteroaryl-heteroalkenyl-, and wherein each of said groups can be unsubstituted or substituted with one or more substituents each independently selected from the group comprising halogen, nitro, oxo, alkyloxy, —C(O)OH, —NH₂, hydroxycarbonylalkyl, hydroxycarbonylalkenyl, hydroxyl, alkyl, alkenyl, heteroalkyl, heteroalkenyl ═S, —SH, aryl, nitroaryl-, nitroaryl-NH, heteroaryl, and heterocyclyl.

Preferred statements and embodiments of the compounds as identified and defined herein, as Rel modulators are set herein below. Each statement or embodiment of a compound (Rel modulator) so defined may be combined with any other statement and/or embodiment, unless clearly indicated to the contrary. In particular, any feature indicated as being preferred or advantageous may be combined with any other features or statements indicated as being preferred or advantageous. Hereto, embodiments of compounds as identified herein as Rel modulators are in particular captured by any one or any combination of one or more of the below numbered statements and embodiments, with any other aspect and/or embodiment.

Statement 1: Modulator of Rel hydrolase and/or synthetase activity, wherein said modulator is a compound of formula (I),

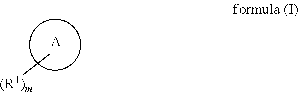

or an isomer, preferably a stereo-isomer or a tautomer, a solvate, a salt, preferably a pharmaceutically acceptable salt, or a prodrug thereof,
wherein m is an integer selected from 1, 2, 3, 4, 5, 6, or 7;
wherein each R¹ is independently selected from halogen, ═O, nitro, or a group comprising hydroxyl, —SH, —NH₂, C(O)OH, heterocyclyl, heteroaryl, C₁₋₆alkyl, haloC₁₋₆alkyl, C₃₋₁₈cycloalkyl, cycloalkenyl, hydroxycarbonylC₁₋₆alkyl, hydroxycarbonylC₂₋₆alkenyl, C₂₋₆alkenyl, C₅₋₁₂aryl, heteroC₁₋₆alkyl, heteroC₂₋₆alkenyl, C₁₋₆alkyloxy, C₅₋₁₂arylC₁₋₆alkyl, C₅₋₁₂arylC₂₋₆alkenyl-, C₅₋₁₂aryl-heteroC₁₋₆alkyl-, C₅₋₁₂aryl-heteroC₂₋₆alkenyl-, C₅₋₁₂aryl-heteroaryl-; C₅₋₁₂aryl-heterocyclyl-, heterocyclyl-C₁₋₆alkyl-, heterocyclyl-C₂₋₆alkenyl-, heterocyclyl-heteroC₁₋₆alkyl-, heterocyclyl-heteroC₂₋₆alkenyl-, heteroaryl-C₁₋₆alkyl-, heteroaryl-C₁₋₆alkenyl-, heteroaryl-heteroC₁₋₆alkyl-, heteroaryl-heteroC₂₋₆alkenyl-, C₁₋₆alkyloxy-, haloC₁₋₆alkoxy-, C₂₋₆alkenyloxy-, C₅₋₁₂aryloxy-; heteroaryloxy-, heterocylyloxy-, C₁₋₆alkylthio-, C₂₋₆alkenylthio-, C₅₋₁₂arylthio-, heteroarylthio-, heterocyclylthio-, C₅₋₁₂aryl-C₁₋₆alkyloxy-, heteroaryl-C₁₋₆alkyloxy-, heterocyclyl-C₁₋₆alkyloxy-, C₅₋₁₂aryl-C₁₋₆alkylthio-, heteroaryl-C₁₋₆alkylthio-, heterocyclyl-C₁₋₆alkylthio-, C₁₋₆alkyl-SO2-, C₂₋₆alkenyl-SO2-, heteroC₁₋₆alkyl-SO2-, heteroC₂₋₆alkenyl-SO2-, C₅₋₁₂aryl-SO2-, heteroaryl-SO2-, heterocyclyl-SO2-, Heteroaryl-heteroC₁₋₆alkyl-heteroaryl-, Heteroaryl-heteroC₂₋₆alkenyl-heteroaryl-, heteroaryl-C₁₋₆alkyl-heteroaryl-, Heteroaryl-C₂₋₆alkenyl-heteroaryl-, C₅₋₁₂Aryl-heteroaryl-C₁₋₆alkyl-, C₅₋₁₂aryl-heteroaryl-heteroC₁₋₆alkyl-, C₅₋₁₂aryl-heteroaryl-C₂₋₆alkenyl-, C₅₋₁₂aryl-heteroaryl-heteroC₂₋₆alkenyl-, C₁₋₆Alkyloxy-C₅₋₁₂aryl-C₁₋₆alkyl-, C₁₋₆Alkyloxy-heteroaryl-C₁₋₆alkyl-, C₁₋₆Alkyloxy-C₅₋₁₂aryl-C₂₋₆alkenyl-, C₁₋₆Alkyloxy-heteroaryl-C₂₋₆alkenyl-, C₁₋₆Alkyloxy-heterocyclyl-C₁₋₆alkyl-, C₁₋₆Alkyloxy-heterocyclyl-C₂₋₆alkenyl-, C₁₋₆Alkyloxy-heterocyclyl-heteroC₁₋₆alkyl-, C₁₋₆Alkyloxy-heterocyclyl-heteroC₂₋₆alkenyl-, C_{5-1 2}aryl-C₂₋₆alkenyl-heteroaryl-heteroC₁₋₆alkyl, C₅₋₁₂aryl-C₁₋₆alkyl-heterocyclyl-heteroC₁₋₆alkyl, C₅₋₁₂Aryl-heteroC₁₋₆alkyl-heteroaryl-, C₅₋₁₂aryl-heteroC₂₋₆alkenyl-heteroaryl-, C₅₋₁₂aryl-heteroC₁₋₆alkyl-heterocyclyl-, C₅₋₁₂aryl-heteroaryl-heterocyclyl-; C₅₋₁₂Aryl-heteroC₁₋₆alkyl-heteroaryl-C₁₋₆alkyl-, C₂₋₆alkenyl-C₅₋₁₂aryl-heteroC₂₋₆alkenyl, C₅₋₁₂Aryl-C₁₋₆alkyl-heterocyclyl-SO2-, C₅₋₁₂aryl-C₂₋₆alkenyl-heterocyclyl-SO2-, heteroaryl-C₁₋₆alkyl-heterocyclyl-SO2-, heteroaryl-C₂₋₆alkenyl-heterocyclyl-SO2-, C₅₋₁₂Aryl-heteroC₂₋₆alkenyl-heteroaryl-C₁₋₆alkyl-, C₂₋₆alkenyl-C₅₋₁₂aryl-heteroC₂₋₆alkenyl-, C₅₋₁₂Aryl-C₁₋₆Alkyl-heterocyclyl-C₁₋₆alkyl-, C₅₋₁₂Aryl-C₁₋₆Alkyl-heterocyclyl-C₂₋₆alkenyl-, C₅₋₁₂Aryl-C₁₋₆Alkyl-heterocyclyl-heteroC₁₋₆alkyl-, C₅₋₁₂Aryl-C₁₋₆Alkyl-heterocyclyl-heteroC₂₋₆alkenyl-, C₅₋₁₂Aryl-C₂₋₆alkenyl-heterocyclyl-C₁₋₆alkyloxy-, C₅₋₁₂Aryl-C₁₋₆alkyl-heterocyclyl-C₁₋₆alkyloxy-, C₅₋₁₂Aryl-C₂₋₆alkenyl-heteroaryl-C₁₋₆alkyloxy-, C₅₋₁₂Aryl-C₁₋₆alkyl-heteroaryl-C₁₋₆alkyloxy-, C₅₋₁₂aryl-imino-, heteroC₁₋₆alkyl-C₅₋₁₂aryl-imino-, C₂₋₆alkenyl-C₅₋₁₂aryl-imino-; and wherein each of said groups can be unsubstituted or substituted with one or more substituents each independently selected from the group comprising halogen, nitro, oxo, C₁₋₆alkyloxy, —C(O)OH, —NH₂, hydroxycarbonylC₁₋₆alkyl, hydroxycarbonylC₂₋₆alkenyl, hydroxyl, C₁₋₆alkyl, C₂₋₆alkenyl, haloC₁₋₆alkyl, haloC₂₋₆alkenyl, heteroC₁₋₆alkyl, heteroC₂₋₆alkenyl, ═S, —SH, C₅₋₁₂aryl, nitroC₅₋₁₂aryl-, heteroaryl, heterocyclyl; C₅₋₁₂aryl-C₁₋₆alkyl-; C₅₋₁₂aryl-C₂₋₆alkenyl-; C₅₋₁₂arylheteroC₁₋₆alkyl-; C₅₋₁₂arylheteroC₂₋₆alkenyl-; heterocyclyl-C₁₋₆alkyl-imino, C₅₋₁₂aryl-imino-, heteroC₁₋₆alkyl-C₅₋₁₂aryl-imino-; and
wherein cycle A is selected from the group represented by formula (Ia);

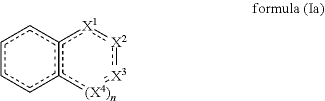

wherein the dotted line represents an optional double bond;
wherein n is an integer selected from 0 or 1;
wherein each of X¹, X², X³, and X⁴ is independently selected from the group comprising N, NH, S, O, C═O, C═S, CH, C(Z¹)₂, and N(Z²), and
   wherein each Z¹ is independently selected from selected from the group comprising hydrogen, halogen, nitro, hydroxyl, —SH, —NH2, —C(O)OH, C₁₋₆alkyl, C₂₋₆alkenyl, hydroxycarbonyl C₁₋₆alkyl, hydroxycarbonylC₂₋₆alkenyl, heteroC₁₋₆alkyl, heteroC₂₋₆alkenyl, C₅₋₁₂aryl, heteroaryl, heterocyclyl, C₁₋₆alkyloxy, C₁₋₆alkylthio, C₅₋₁₂arylC₁₋₆alkyl-, C₅₋₁₂arylC₂₋₆alkenyl-, C₅₋₁₂aryl-heteroC₁₋₆alkyl-, C₅₋₁₂aryl-heteroC₂₋₆alkenyl-, heterocyclyl-heteroC₁₋₆alkyl, heterocyclyl-heteroC₂₋₆alkenyl-, heteroaryl-heteroC₁₋₆alkyl-, heteroaryl-heteroC₂₋₆alkenyl-, Heteroaryl-heteroC₁₋₆alkyl-heteroaryl-, Heteroaryl-heteroC₂₋₆alkenyl-heteroaryl-, C₅₋₁₂Aryl-heteroaryl-C₁₋₆alkyl-, C₅₋₁₂Aryl-heteroaryl-heteroC₁₋₆alkyl-, C₅₋₁₂Aryl-heteroaryl-C₂₋₆alkenyl, C₅₋₁₂Aryl-heteroaryl-heteroC₂₋₆alkenyl, C₁₋₆Alkyloxy-aryl-C₁₋₆alkyl-, and C₁₋₆Alkyloxy-aryl-C₂₋₆alkenyl-; and
   wherein each Z² is independently selected from the group comprising C₁₋₆alkyl, C₂₋₆alkenyl, hydroxycarbonylC₁₋₆alkyl, hydroxycarbonylC₂₋₆alkenyl, heteroC₁₋₆alkyl, heteroC₂₋₆alkenyl, C₅₋₁₂aryl, heteroaryl, heterocyclyl, C₅₋₁₂C5-12aryl-heteroC₁₋₆alkyl-, C₅₋₁₂aryl-heteroC₂₋₆alkenyl-, heterocyclyl-heteroC₁₋₆alkyl, heterocyclyl-heteroC₂₋₆alkenyl-, heteroaryl-heteroC₁₋₆alkyl-, heteroaryl-heteroC₂₋₆alkenyl-, Heteroaryl-heteroC₁₋₆alkyl-heteroaryl-, Heteroaryl-heteroC₂₋₆alkenyl-heteroaryl-, C₅₋₁₂Aryl-heteroaryl-C₁₋₆alkyl-, C₅₋₁₂Aryl-heteroaryl-heteroC₁₋₆alkyl-, C₅₋₁₂Aryl-heteroaryl-C₂₋₆alkenyl, C₅₋₁₂Aryl-heteroaryl-heteroC₂₋₆alkenyl, C₁₋₆Alkyloxy-aryl-C₁₋₆alkyl-, and C₁₋₆Alkyloxy-aryl-C₂₋₆alkenyl-; or
wherein when X² and X³ are each independently selected from C(Z¹)₂ or N(Z²) as defined above, two Z¹, or Z¹ together with Z² together with the atom to which they are attached form a ring selected from the group comprising heterocyclyl, cycloalkyl, cycloalkenyl, C₅₋₁₂aryl, and heteroaryl.

Statement 2: The modulator according to statement 1, wherein said modulator is a compound of formula (I),

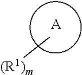

wherein m is an integer selected from 1, 2, 3, 4, or 5;
wherein each R¹ is independently selected from halogen, ═O, nitro, or a group comprising hydroxyl, C(O)OH, heterocyclyl, heteroaryl, C₁₋₆alkyl, hydroxycarbonylC₁₋₆alkyl, hydroxycarbonylC₂₋₆alkenyl, C₂₋₆alkenyl, C₅₋₁₂aryl, heteroC₁₋₆alkyl, heteroC₂₋₆alkenyl, C₁₋₆alkyloxy, C₅₋₁₂arylC₁₋₆alkyl, C₅₋₁₂arylC₂₋₆alkenyl-, C₅₋₁₂aryl-heteroC₁₋₆alkyl-, C₅₋₁₂aryl-heteroC₂₋₆alkenyl-, heterocyclyl-C₁₋₆alkyl-, heterocyclyl-C₂₋₆alkenyl-, heterocyclyl-heteroC₁₋₆alkyl-, heterocyclyl-heteroC₂₋₆alkenyl-, heteroaryl-C₁₋₆alkyl-, heteroaryl-C₁₋₆alkenyl-, heteroaryl-heteroC₁₋₆alkyl-, heteroaryl-heteroC₂₋₆alkenyl-, C₁₋₆alkyl-SO2-, heteroC₁₋₆alkyl-SO2-, C₅₋₁₂aryl-C₁₋₆alkyl-heterocyclyl-SO2-, heterocyclyl-SO2-, Heteroaryl-heteroC₁₋₆alkyl-heteroaryl-, Heteroaryl-heteroC₂₋₆alkenyl-heteroaryl-, Heteroaryl-C₂₋₆alkenyl-heteroaryl-, C₅₋₁₂Aryl-heteroaryl-C₁₋₆alkyl-, C₅₋₁₂aryl-heteroaryl-heteroC₂₋₆alkenyl-, C₁₋₆Alkyloxy-C₅₋₁₂aryl-C₂₋₆alkenyl-, C₁₋₆Alkyloxy-heterocyclyl-heteroC₁₋₆alkyl-, C₅₋₁₂aryl-C₂₋₆alkenyl-heteroaryl-heteroC₁₋₆alkyl, C₅₋₁₂aryl-C₁₋₆alkyl-heterocyclyl-heteroC₁₋₆alkyl, C₅₋₁₂Aryl-heteroC₁₋₆alkyl-heteroaryl-C₁₋₆alkyl-, C₂₋₆alkenyl-C₅₋₁₂aryl-heteroC₂₋₆alkenyl, C₅₋₁₂aryl-imino-, heteroC₁₋₆alkyl-C₅₋₁₂aryl-imino-, C₂₋₆alkenyl-C₅₋₁₂aryl-imino-, and wherein each of said groups can be unsubstituted or substituted with one or more substituents each independently selected from the group comprising halogen, nitro, oxo, C₁₋₆alkyloxy, —C(O)OH, hydroxyl, hydroxycarbonylC₁₋₆alkyl-, hydroxycarbonylC₂₋₆alkenyl-, C₁₋₆alkyl, C₂₋₆alkenyl, C₅₋₁₂aryl, and nitroC₅₋₁₂aryl; and
wherein cycle A is selected from the group represented by formula (Ia);

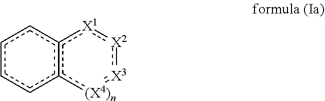

wherein the dotted line represents an optional double bond;
wherein n is an integer selected from 0 or 1;
wherein each of X¹, X², X³, and X⁴ is independently selected from the group comprising N, NH, S, O, C═O, C═S, CH, C(Z¹)₂, and N(Z²), and
   wherein each Z¹ is independently selected from selected from the group comprising hydrogen, halogen, nitro, hydroxyl, —SH, —NH2, —C(O)OH, C₁₋₆alkyl, C₂₋₆alkenyl, hydroxycarbonyl C₁₋₆alkyl, hydroxycarbonylC₂₋₆alkenyl, heteroC₁₋₆alkyl, heteroC₂₋₆alkenyl, C₅₋₁₂aryl, heteroaryl, heterocyclyl, C₁₋₆alkyloxy, C₁₋₆alkylthio, C₅₋₁₂arylC₁₋₆alkyl-, C₅₋₁₂aryl-C₂₋₆alkenyl-, C₅₋₁₂aryl-heteroC₁₋₆alkyl-, C₅₋₁₂aryl-heteroC₂₋₆alkenyl-, heterocyclyl-heteroC₁₋₆alkyl, heterocyclyl-heteroC₂₋₆alkenyl-, heteroaryl-heteroC₁₋₆alkyl-, heteroaryl-heteroC₂₋₆alkenyl-, Heteroaryl-heteroC₁₋₆alkyl-heteroaryl-, Heteroaryl-heteroC₂₋₆alkenyl-heteroaryl-, C₅₋₁₂Aryl-heteroaryl-C₁₋₆alkyl-, C₅₋₁₂Aryl-heteroaryl-heteroC₁₋₆alkyl-, C₅₋₁₂Aryl-heteroaryl-C₂₋₆alkenyl, C₅₋₁₂Aryl-heteroaryl-heteroC₂₋₆alkenyl, C₁₋₆Alkyloxy-aryl-C₁₋₆alkyl-, and C₁₋₆Alkyloxy-aryl-C₂₋₆alkenyl-; and
   wherein each Z² is independently selected from the group comprising C₁₋₆alkyl, C₂₋₆alkenyl, hydroxycarbonylC₁₋₆alkyl, hydroxycarbonylC₂₋₆alkenyl, heteroC₁₋₆alkyl, heteroC₂₋₆alkenyl, C₅₋₁₂aryl, heteroaryl, heterocyclyl, C₅₋₁₂aryl-heteroC₁₋₆alkyl-, C₅₋₁₂aryl-heteroC₂₋₆alkenyl-, heterocyclyl-heteroC₁₋₆alkyl, heterocyclyl-heteroC₂₋₆alkenyl-, heteroaryl-heteroC₁₋₆alkyl-, heteroaryl-heteroC₂₋₆alkenyl-, Heteroaryl-heteroC₁₋₆alkyl-heteroaryl-, Heteroaryl-heteroC₂₋₆alkenyl-heteroaryl-, C₅₋₁₂Aryl-heteroaryl-C₁₋₆alkyl-, C₅₋₁₂Aryl-heteroaryl-heteroC₁₋₆alkyl-, C₅₋₁₂Aryl-heteroaryl-C₂₋₆alkenyl, C₅₋₁₂Aryl-heteroaryl-heteroC₂₋₆alkenyl, C₁₋₆Alkyloxy-aryl-C₁₋₆alkyl-, and C₁₋₆Alkyloxy-aryl-C₂₋₆alkenyl-; or
wherein when X² and X³ are each independently selected from C(Z¹)₂ or N(Z²) as defined above, two Z¹, or Z¹ together with Z² together with the atom to which they are attached form a ring selected from the group comprising heterocyclyl, C₃₋₁₈cycloalkyl, cycloalkenyl, C₅₋₁₂aryl, and heteroaryl.

Statement 3: The modulator according to any of statements 1 to 2, wherein said modulator is a compound of formula (I),

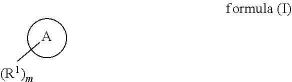

wherein m is an integer selected from 1, 2, 3, 4, or 5;
wherein each R¹ is independently selected from halogen, ═O, nitro, or a group comprising hydroxyl, C(O)OH, heterocyclyl, heteroaryl, C₁₋₆alkyl, hydroxycarbonylC₁₋₆alkyl, hydroxycarbonylC₂₋₆alkenyl, C₂₋₆alkenyl, C₅₋₁₂aryl, heteroC₁₋₆alkyl, heteroC₂₋₆alkenyl, C₁₋₆alkyloxy, C₅₋₁₂arylC₁₋₆alkyl, C₅₋₁₂arylC₂₋₆alkenyl-, C₅₋₁₂aryl-heteroC₁₋₆alkyl-, C₅₋₁₂aryl-heteroC₂₋₆alkenyl-, heterocyclyl-C₁₋₆alkyl-, heterocyclyl-C₂₋₆alkenyl-, heterocyclyl-heteroC₁₋₆alkyl-, heterocyclyl-heteroC₂₋₆alkenyl-, heteroaryl-C₁₋₆alkyl-, heteroaryl-C₁₋₆alkenyl-, heteroaryl-heteroC₁₋₆alkyl-, heteroaryl-heteroC₂₋₆alkenyl-, C₁₋₆alkyl-SO2-, heteroC₁₋₆alkyl-SO2-, C₅₋₁₂aryl-C₁₋₆alkyl-heterocyclyl-SO2-, heterocyclyl-SO2-, Heteroaryl-heteroC₁₋₆alkyl-heteroaryl-, Heteroaryl-heteroC₂₋₆alkenyl-heteroaryl-, Heteroaryl-C₂₋₆alkenyl-heteroaryl-, C₅₋₁₂Aryl-heteroaryl-C₁₋₆alkyl-, C₅₋₁₂aryl-heteroaryl-heteroC₂₋₆alkenyl-, C₁₋₆Alkyloxy-C₅₋₁₂aryl-C₂₋₆alkenyl-, C₁₋₆Alkyloxy-heterocyclyl-heteroC₁₋₆alkyl-, C₅₋₁₂aryl-C₂₋₆alkenyl-heteroaryl-heteroC₁₋₆alkyl, C₅₋₁₂aryl-C₁₋₆alkyl-heterocyclyl-heteroC₁₋₆alkyl, C₅₋₁₂Aryl-heteroC₁₋₆alkyl-heteroaryl-C₁₋₆alkyl-, C₂₋₆alkenyl-C₅₋₁₂aryl-hetero C₂₋₆alkenyl, C₅₋₁₂aryl-imino-, heteroC₁₋₆alkyl-C₅₋₁₂aryl-imino-, C₂₋₆alkenyl-C₅₋₁₂aryl-imino-, and wherein each of said groups can be unsubstituted or substituted with one or more substituents each independently selected from the group comprising halogen, nitro, oxo, C₁₋₆alkyloxy, —C(O)OH, hydroxyl, hydroxycarbonylC₁₋₆alkyl-, hydroxycarbonylC₂₋₆alkenyl-, C₁₋₆alkyl, C₂₋₆alkenyl, C₅₋₁₂aryl, and nitroC₅₋₁₂aryl; and
wherein cycle A is selected from the group represented by formula (Ia);

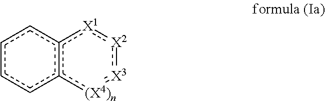

wherein n is 1;
wherein the dotted line represents an optional double bond;
wherein each of X¹, X², X³, and X⁴ is independently selected from the group comprising N, NH, S, O, C═O, C═S, CH, C(Z¹)₂, and N(Z²), and
   wherein each Z¹ is independently selected from selected from the group comprising hydrogen, halogen, nitro, hydroxyl, —SH, —NH2, —C(O)OH, C₁₋₆alkyl, C2-6alkenyl, hydroxycarbonylC1-6alkyl, hydroxycarbonylC2-6alkenyl, heteroC1-6alkyl, heteroC2-6alkenyl, C5-12aryl, heteroaryl, heterocyclyl, C1-6alkyloxy, C1-6alkylthio, C5-12arylC1-6alkyl-, C5-12aryl-C2-6alkenyl-, C5-12aryl-heteroC1-6alkyl-, C5-12aryl-heteroC2-6alkenyl-, heterocyclyl-heteroC1-6alkyl, heterocyclyl-heteroC2-6alkenyl-, heteroaryl-heteroC1-6alkyl-, heteroaryl-heteroC2-6alkenyl-, Heteroaryl-heteroC1-6alkyl-heteroaryl-, Heteroaryl-heteroC2-6alkenyl-heteroaryl-, C5-12Aryl-heteroaryl-C1-6alkyl-, C5-12Aryl-heteroaryl-heteroC1-6alkyl-, C5-12Aryl-heteroaryl-C2-6alkenyl, C5-12Aryl-heteroaryl-heteroC2-6alkenyl, C1-6Alkyloxy-aryl-C1-6alkyl-, and C1-6Alkyloxy-aryl-C2-6alkenyl-; and
   wherein each Z² is independently selected from the group comprising C₁₋₆alkyl, C₂₋₆alkenyl, hydroxycarbonylC1-6alkyl, hydroxycarbonylC2-6alkenyl, heteroC1-6alkyl, heteroC2-6alkenyl, C5-12aryl, heteroaryl, heterocyclyl, C5-12aryl-heteroC1-6alkyl-, C5-12aryl-heteroC2-6alkenyl-, heterocyclyl-heteroC1-6alkyl, heterocyclyl-heteroC2-6alkenyl-, heteroaryl-heteroC1-6alkyl-, heteroaryl-heteroC2-6alkenyl-, Heteroaryl-heteroC1-6alkyl-heteroaryl-, Heteroaryl-heteroC2-6alkenyl-heteroaryl-, C5-12Aryl-heteroaryl-C1-6alkyl-, C5-12Aryl-heteroaryl-heteroC1-6alkyl-, C5-12Aryl-heteroaryl-C2-6alkenyl, C5-12Aryl-heteroaryl-heteroC2-6alkenyl, C1-6Alkyloxy-aryl-C1-6alkyl-, and C1-6Alkyloxy-aryl-C2-6alkenyl-; or
wherein when X² and X³ are each independently selected from C(Z¹)₂ or N(Z²) as defined above, two Z¹, or Z¹ together with Z² together with the atom to which they are attached form a ring selected from the group comprising heterocyclyl, C₃₋₁₈cycloalkyl, cycloalkenyl, C₅₋₁₂aryl, and heteroaryl.

Statement 4: The modulator according to any of statements 1 to 3 wherein said modulator is a compound of formula (I),

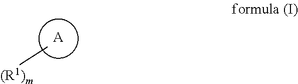

wherein m is an integer selected from 1, 2, 3, 4, or 5;
wherein each R¹ is independently selected from halogen, ═O, nitro, or a group comprising hydroxyl, C(O)OH, heterocyclyl, heteroaryl, C₁₋₆alkyl, hydroxycarbonylC₁₋₆alkyl, hydroxycarbonylC₂₋₆alkenyl, C₂₋₆alkenyl, C₅₋₁₂aryl, heteroC₁₋₆alkyl, heteroC₂₋₆alkenyl, C₁₋₆alkyloxy, C₅₋₁₂arylC₁₋₆alkyl, C₅₋₁₂arylC₂₋₆alkenyl-, C₅₋₁₂aryl-heteroC₁₋₆alkyl-, C₅₋₁₂aryl-heteroC₂₋₆alkenyl-, heterocyclyl-C₁₋₆alkyl-, heterocyclyl-C₂₋₆alkenyl-, heterocyclyl-heteroC₁₋₆alkyl-, heterocyclyl-heteroC₂₋₆alkenyl-, heteroaryl-C₁₋₆alkyl-, heteroaryl-C₁₋₆alkenyl-, heteroaryl-heteroC₁₋₆alkyl-, heteroaryl-heteroC₂₋₆alkenyl-, C₁₋₆alkyl-SO2-, heteroC₁₋₆alkyl-SO2-, C₅₋₁₂aryl-C₁₋₆alkyl-heterocyclyl-SO2-, heterocyclyl-SO2-, Heteroaryl-heteroC₁₋₆alkyl-heteroaryl-, Heteroaryl-heteroC₂₋₆alkenyl-heteroaryl-, Heteroaryl-C₂₋₆alkenyl-heteroaryl-, C₅₋₁₂Aryl-heteroaryl-C₁₋₆alkyl-, C₅₋₁₂aryl-heteroaryl-heteroC₂₋₆alkenyl-, C₁₋₆Alkyloxy-C₅₋₁₂aryl-C₂₋₆alkenyl-, C₁₋₆Alkyloxy-heterocyclyl-heteroC₁₋₆alkyl-, C₅₋₁₂aryl-C₂₋₆alkenyl-heteroaryl-heteroC₁₋₆alkyl, C₅₋₁₂aryl-C₁₋₆alkyl-heterocyclyl-heteroC₁₋₆alkyl, C₅₋₁₂Aryl-heteroC₁₋₆alkyl-heteroaryl-C₁₋₆alkyl-, C₂₋₆alkenyl-C₅₋₁₂aryl-heteroC₂₋₆alkenyl, C₅₋₁₂aryl-imino-, heteroC₁₋₆alkyl-C₅₋₁₂aryl-imino-, C₂₋₆alkenyl-C₅₋₁₂aryl-imino-, and wherein each of said groups can be unsubstituted or substituted with one or more substituents each independently selected from the group comprising halogen, nitro, oxo, C₁₋₆alkyloxy, —C(O)OH, hydroxyl, hydroxycarbonylC₁₋₆alkyl-, hydroxycarbonylC₂₋₆alkenyl-, C₁₋₆alkyl, C₂₋₆alkenyl, C₅₋₁₂aryl, and nitroC₅₋₁₂aryl; and
wherein cycle A is selected from the group represented by formula (Ia);

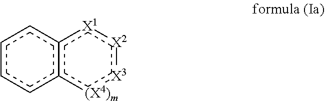

wherein n is 1;
wherein the dotted line represents an optional double bond;
wherein each of X¹, X², X³, and X⁴ is independently selected from the group comprising N, NH, S, O, C═O, C═S, CH, C(Z¹)₂, and N(Z²), and
   wherein each Z¹ is independently selected from the group comprising hydrogen, halogen, nitro, hydroxyl, —SH, —NH₂, —C(O)OH, C₁₋₆alkyl, C₂₋₆alkenyl, hydroxycarbonylC₁₋₆alkyl, hydroxycarbonylC₂₋₆alkenyl, heteroC₁₋₆alkyl, heteroC₂₋₆alkenyl, C₅₋₁₂aryl, C₅₋₁₂arylC₁₋₆alkyl-, heteroaryl, heterocyclyl, C₁₋₆alkyloxy, and C₁₋₆alkylthio; and
   wherein each Z² is independently selected from the group comprising C₁₋₆alkyl, C₂₋₆alkenyl, hydroxycarbonylC₁₋₆alkyl, hydroxycarbonylC₂₋₆alkenyl, heteroC₁₋₆alkyl, heteroC₂₋₆alkenyl, C₅₋₁₂aryl, heteroaryl, and heterocyclyl, or
wherein when X² and X³ are each independently selected from C(Z¹)₂ or N(Z²) as defined above, two Z¹, or Z¹ together with Z² together with the atom to which they are attached form a ring selected from the group comprising heterocyclyl, C₃₋₁₈cycloalkyl, cycloalkenyl, C₅₋₁₂aryl, and heteroaryl.

Statement 5: The modulator according to any of statements 1 to 4, wherein said modulator is any of compounds 1 to 24 as selected from Table A. Yes/No in the 5^{th} and 6^{th} columns indicates whether there is, or respectively is not an inhibition of the synthetase (ST) and/or hydrolase (HD) activity.

TABLE A

| Compound | | | | | |
|---|---|---|---|---|---|
| # | Molport ID | ZINC ID | ST | HD | |
| 1 | MolPort- 002-360-101 | ZINC0000197 01838 | No | Yes | 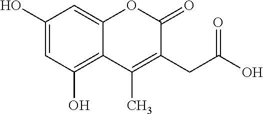 [SMILES: Cc1c(CC(=O)O)c(=O)oc2cc(O)cc(O)c12] |
| 2 | MolPort- 002-521-254 | ZINC0000040 63453 | No | Yes | 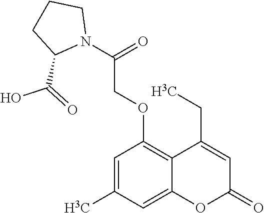 [SMILES: [3CH3]Cc1cc(=O)oc2cc([3CH3])cc(OCC(=O)N3CCC[C@H]3C(=O)O)c12] |
| 3 | MolPort- 002-467-279 | ZINC0000042 00302 | No | Yes | 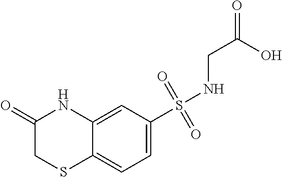 [SMILES: O=C(O)CNS(=O)(=O)c1ccc2c(c1)NC(=O)CS2] |
| 4 | MolPort- 006-316-947 | ZINC0000204 26247 | No | Yes | 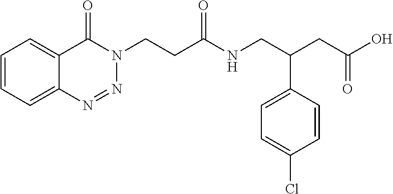 [SMILES: O=C(O)CC(CNC(=O)CCn1nnc2ccccc2c1=O)c1ccc(Cl)cc1] |
| 5 | MolPort- 002-771-642 | ZINC0000198 52658 | No | Yes | 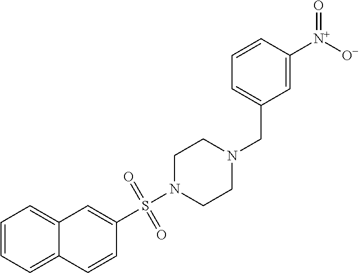 [SMILES: O=[N+]([O-])c1cccc(CN2CCN(S(=O)(=O)c3ccc4ccccc4c3)CC2)c1] |
| 6 | MolPort- 039-295-727 | ZINC0002380 32832 | No | Yes | 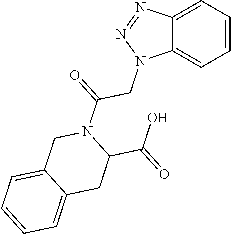 [SMILES: O=C(O)C1Cc2ccccc2CN1C(=O)Cn1nnc2ccccc21] |
| 7 | MolPort- 005-744-141 | ZINC0000114 51159 | No | Yes | 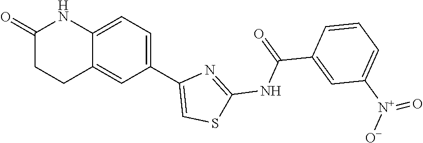 [SMILES: O=C1CCc2cc(-c3csc(NC(=O)c4cccc([N+](=O)[O-])c4)n3)ccc2N1] |
| 8 | MolPort- 009-605-967 | ZINC0000979 71241 | No | Yes | 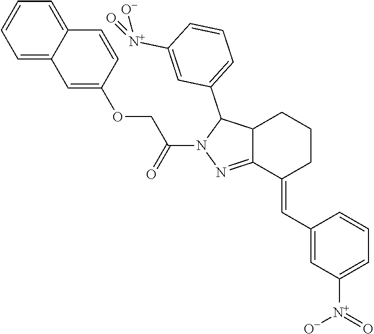 [SMILES: O=C(COc1ccc2ccccc2c1)N1N=C2/C(=C/c3cccc([N+](=O)[O-])c3)CCCC2C1c1cccc([N+](=O)[O-])c1] |
| 9 | MolPort- 005-910-590 | ZINC0000129 02007 | no | yes | 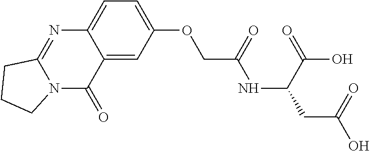 [SMILES: O=C(O)C[C@H](NC(=O)COc1ccc2nc3n(c(=O)c2c1)CCC3)C(=O)O] |
| 10 | MolPort- 001-988-631 | ZINC0000000 52427 | Yes | No | 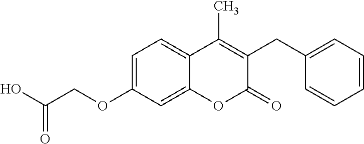 [SMILES: Cc1c(Cc2ccccc2)c(=O)oc2cc(OCC(=O)O)ccc12] |
| 11 | MolPort- 000-851-674 | ZINC0000004 54681 | Yes | No | 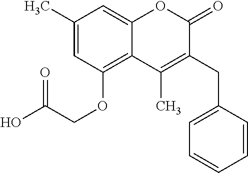 [SMILES: Cc1cc(OCC(=O)O)c2c(C)c(Cc3ccccc3)c(=O)oc2c1] |
| 12 | MolPort- 002-646-536 | ZINC0000042 82624 | Yes | No | 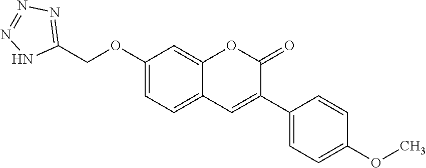 [SMILES: COc1ccc(-c2cc3ccc(OCc4nnn[nH]4)cc3oc2=O)cc1] |
| 13 | MolPort- 002-639-453 | ZINC0000061 96170 | Yes | No | 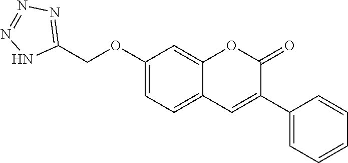 [SMILES: O=c1oc2cc(OCc3nnn[nH]3)ccc2cc1-c1ccccc1] |
| 14 | MolPort- 002-646-803 | ZINC0000062 25332 | Yes | No | 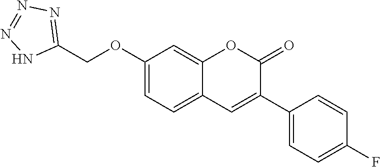 [SMILES: O=c1oc2cc(OCc3nnn[nH]3)ccc2cc1-c1ccc(F)cc1] |
| 15 | MolPort- 002-208-461 | ZINC0000006 22142 | Yes | No | 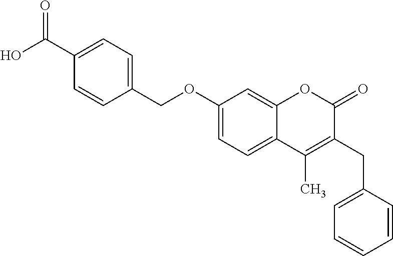 [SMILES: Cc1c(Cc2ccccc2)c(=O)oc2cc(OCc3ccc(C(=O)O)cc3)ccc12] |
| 16 | MolPort- 006-317-247 | ZINC0000138 04391 | Yes | Yes | 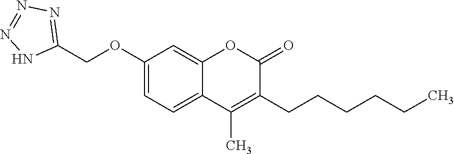 [SMILES: CCCCCCc1c(C)c2ccc(OCc3nnn[nH]3)cc2oc1=O] |
| 17 | MolPort- 005-977-395 | ZINC0000131 15587 | Yes | Yes | 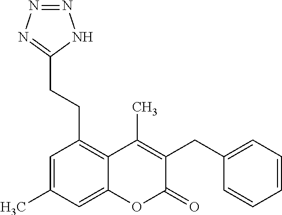 [SMILES: Cc1cc(CCc2nnn[nH]2)c2c(C)c(Cc3ccccc3)c(=O)oc2c1] |
| 18 | MolPort- 005-979-219 | ZINC0000060 59515 | Yes | Yes | 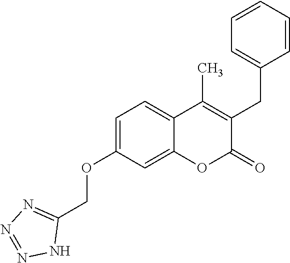 [SMILES: Cc1c(Cc2ccccc2)c(=O)oc2cc(OCc3nnn[nH]3)ccc12] |
| 19 | MolPort- 005-971-847 | ZINC0000024 50594 | Yes | Yes | 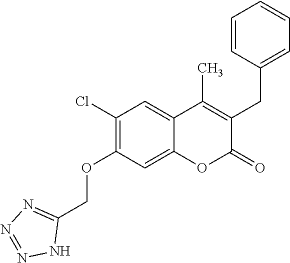 [SMILES: Cc1c(Cc2ccccc2)c(=O)oc2cc(OCc3nnn[nH]3)c(Cl)cc12] |
| 20 | MolPort- 002-643-495 | ZINC0000062 25320 | Yes | Yes | 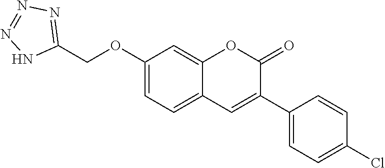 [SMILES: O=c1oc2cc(OCc3nnn[nH]3)ccc2cc1-c1ccc(Cl)cc1] |
| 21 | MolPort- 002-636-616 | ZINC0000061 40623 | Yes | Yes | 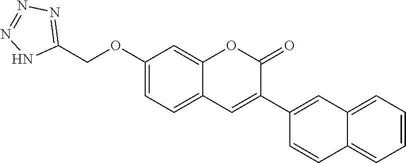 [SMILES: O=c1oc2cc(OCc3nnn[nH]3)ccc2cc1-c1ccc2ccccc2c1] |
| 22 | MolPort- 039-321-539 | ZINC0003300 32877 | Yes | Yes | 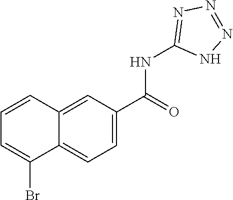 [SMILES: O=C(Nc1nnn[nH]1)c1ccc2c(Br)cccc2c1] |
| 23 | MolPort- 005-654-010 | ZINC0000127 72125 | Yes | Yes | 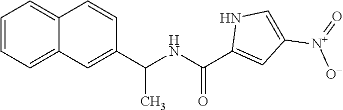 [SMILES: CC(NC(=O)c1cc([N+](=O)[O-])c[nH]1)c1ccc2ccccc2c1] |
| 24 | MolPort- 000-453-834 | ZINC0000093 56746 | Yes | Yes | 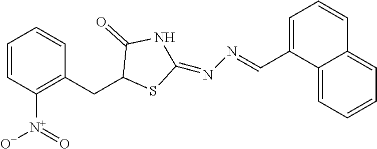 [SMILES: O=C1N/C(=N\N=C\c2cccc3ccccc23)SC1Cc1ccccc1[N+](=O)[O-]] |

Statement 6: The modulator according to any of statements 1 to 3, wherein said modulator is a compound of formula (I),

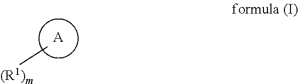

wherein m is an integer selected from 1, 2, 3, 4, or 5;

wherein each R¹ is independently selected from halogen, ═O, nitro, or a group comprising hydroxyl, heterocyclyl, heteroaryl, C₁₋₆alkyl, C₂₋₆alkenyl, C₅₋₁₂aryl, heteroC₁₋₆alkyl, heteroC₂₋₆alkenyl, C₅₋₁₂arylC₁₋₆alkyl, C₅₋₁₂arylC₂₋₆alkenyl-, C₅₋₁₂aryl-heteroC₁₋₆alkyl-, C₅₋₁₂aryl-heteroC₂₋₆alkenyl-, heterocyclyl-C₁₋₆alkyl-, heterocyclyl-heteroC₁₋₆alkyl-, heterocyclyl-C₂₋₆alkenyl-, heterocyclyl-heteroC₂₋₆alkenyl-, heteroaryl-C₁₋₆alkyl-, heteroaryl-C₁₋₆alkenyl-, heteroaryl-heteroC₁₋₆alkyl-, heteroaryl-heteroC₂₋₆alkenyl-, C₁₋₆alkyl-SO2-, heteroC₁₋₆alkyl-SO2-, heterocyclyl-SO2-, Heteroaryl-heteroC₁₋₆alkyl-heteroaryl-, Heteroaryl-heteroC₂₋₆alkenyl-heteroaryl-, Heteroaryl-C₂₋₆alkenyl-heteroaryl-, C₅₋₁₂Aryl-heteroaryl-C₁₋₆alkyl-, C₅₋₁₂aryl-heteroaryl-heteroC₂₋₆alkenyl-, C₁₋₆Alkyloxy-C₅₋₁₂aryl-C₂₋₆alkenyl-, C₁₋₆Alkyloxy-heterocyclyl-heteroC₁₋₆alkyl-, C₅₋₁₂Aryl-heteroC₁₋₆alkyl-heteroaryl-C₁₋₆alkyl-, C₂₋₆alkenyl-C₅₋₁₂aryl-heteroC₂₋₆alkenyl, C₅₋₁₂aryl-imino-, heteroC₁₋₆alkyl-C₅₋₁₂aryl-imino-, C₂₋₆alkenyl-C₅₋₁₂aryl-imino-, and wherein each of said groups can be unsubstituted or substituted with one or more substituents each independently selected from the group comprising halogen, nitro, oxo, C₁₋₆alkyloxy, —C(O)OH, hydroxyl, hydroxycarbonylC₁₋₆alkyl-, hydroxycarbonylC₂₋₆alkenyl-, C₁₋₆alkyl, C₂₋₆alkenyl, and C₅₋₁₂aryl;

wherein cycle A is selected from the group represented by formula (Ib);

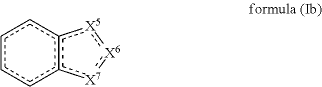

wherein the dotted line represents an optional double bond;
wherein each of X⁵, X⁶, and X⁷ is independently selected from the group comprising N, NH, S, O, C═O, C═S, CH, C(Z¹)₂, and N(Z²), and
   wherein each Z¹ is independently selected from selected from the group comprising hydrogen, halogen, nitro, hydroxyl, —SH, —NH2, —C(O)OH, C₁₋₆alkyl, C₂₋₆alkenyl, hydroxycarbonylC₁₋₆alkyl, hydroxycarbonylC₂₋₆alkenyl, heteroC₁₋₆alkyl, heteroC₂₋₆alkenyl, C₅₋₁₂aryl, heteroaryl, heterocyclyl, C₁₋₆alkyloxy, C₁₋₆alkylthio, C₅₋₁₂arylC₁₋₆alkyl-, C₅₋₁₂aryl-C₂₋₆alkenyl-, C₅₋₁₂aryl-heteroC₁₋₆alkyl-, C₅₋₁₂aryl-heteroC₂₋₆alkenyl-, heterocyclyl-heteroC₁₋₆alkyl, heterocyclyl-heteroC₂₋₆alkenyl-, heteroaryl-heteroC₁₋₆alkyl-, heteroaryl-heteroC₂₋₆alkenyl-, Heteroaryl-heteroC₁₋₆alkyl-heteroaryl-Heteroaryl-heteroC₂₋₆alkenyl-heteroaryl-, C₅₋₁₂Aryl-heteroaryl-C₁₋₆alkyl-, C₅₋₁₂Aryl-heteroaryl-heteroC₁₋₆alkyl-, C₅₋₁₂Aryl-heteroaryl-C₂₋₆alkenyl, C₅₋₁₂Aryl-heteroaryl-heteroC₂₋₆alkenyl, C₁₋₆Alkyloxy-aryl-C₁₋₆alkyl-, and C₁₋₆Alkyloxy-aryl-C₂₋₆alkenyl-; and
   wherein each Z²is independently selected from the group comprising C₁₋₆alkyl, C₂₋₆alkenyl, hydroxycarbonylC₁₋₆alkyl, hydroxycarbonylC₂₋₆alkenyl, heteroC₁₋₆alkyl, heteroC₂₋₆alkenyl, C₅₋₁₂aryl, heteroaryl, heterocyclyl, C₅₋₁₂aryl-heteroC₁₋₆alkyl-, C₅₋₁₂aryl-heteroC₂₋₆alkenyl-, heterocyclyl-heteroC₁₋₆alkyl, heterocyclyl-heteroC₂₋₆alkenyl-, heteroaryl-heteroC₁₋₆alkyl-, heteroaryl-heteroC₂₋₆alkenyl-, Heteroaryl-heteroC₁₋₆alkyl-heteroaryl-, Heteroaryl-heteroC₂₋₆alkenyl-heteroaryl-, C₅₋₁₂Aryl-heteroaryl-C₁₋₆alkyl-, C₅₋₁₂Aryl-heteroaryl-heteroC₁₋₆alkyl-, C₅₋₁₂Aryl-heteroaryl-C₂₋₆alkenyl, C₅₋₁₂Aryl-heteroaryl-heteroC2-6alkenyl, C₁₋₆Alkyloxy-aryl-C₁₋₆alkyl-, and C₁₋₆Alkyloxy-aryl-C₂₋₆alkenyl-.

Statement 7: The modulator according to any of statements 1 to 3 and 6, wherein said modulator is a compound of formula (I),

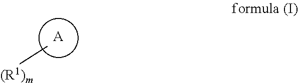

wherein m is an integer selected from 1, 2, 3, 4, or 5; and preferably from 1, 2, 3 or 4

wherein each R¹ is independently selected from halogen, ═O, nitro, or a group comprising hydroxyl, heteroaryl, heterocyclyl, heteroC₁₋₆alkyl, heteroC₂₋₆alkenyl, C₁₋₆alkyl, C₂₋₆alkenyl, C₅₋₁₂aryl, C₅₋₁₂arylC₁₋₆alkyl, C₅₋₁₂arylC₂₋₆alkenyl-, C₅₋₁₂aryl-heteroC₁₋₆alkyl-, C₅₋₁₂aryl-heteroC₂₋₆alkenyl-, heterocyclyl-C₁₋₆alkyl-, heterocyclyl-heteroC₁₋₆alkyl-, heterocyclyl-C₂₋₆alkenyl-, heterocyclyl-heteroC₂₋₆alkenyl-, heteroaryl-C₁₋₆alkyl-, heteroaryl-C₁₋₆alkenyl-, heteroaryl-heteroC₁₋₆alkyl-, heteroaryl-heteroC₂₋₆alkenyl-, SO2-, heteroC₁₋₆alkyl-SO2-, heterocyclyl-SO2-, Heteroaryl-heteroC₁₋₆alkyl-hetero Heteroaryl-heteroC₂₋₆alkenyl-heteroaryl-, Heteroaryl-C₂₋₆alkenyl-heteroaryl-, C₅₋₁₂Aryl-heteroaryl-C₁₋₆alkyl-, C₅₋₁₂aryl-heteroaryl-heteroC₂₋₆alkenyl-, C₁₋₆Alkyloxy-C₅₋₁₂aryl-C₂₋₆alkenyl-, C₁₋₆Alkyloxy-heterocyclyl-heteroC₁₋₆alkyl-, C_{5-1 2}Aryl-heteroC₁₋₆alkyl-heteroaryl-C₁₋₆alkyl-, C₂₋₆alkenyl-C₅₋₁₂aryl-heteroC₂₋₆alkenyl, C₅₋₁₂aryl-imino-, heteroC₁₋₆alkyl-C₅₋₁₂-aryl-imino-, C₂₋₆alkenyl-C₅₋₁₂aryl-imino-, and wherein each of said groups can be unsubstituted or substituted with one or more substituents each independently selected from the group comprising halogen, nitro, oxo, C₁₋₆alkyloxy, —C(O)OH, hydroxyl, hydroxycarbonylC₁₋₆alkyl-, hydroxycarbonylC₂₋₆alkenyl-, C₁₋₆alkyl, C₂₋₆alkenyl, and C₅₋₁₂aryl, and

wherein cycle A is selected from the group represented by formula (Ib);

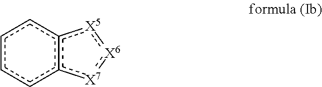

wherein the dotted line represents an optional double bond;
wherein each of X⁵, X⁶, and X⁷ is independently selected from the group comprising N, NH, S, O, C═O, C═S, CH, C(Z¹)₂, and N(Z²), and
   wherein each Z¹ is independently selected from the group comprising hydrogen, halogen, nitro, hydroxyl, —SH, —NH₂, —C(O)OH, C₁₋₆alkyl, C₂₋₆alkenyl, hydroxycarbonylC₁₋₆alkyl, hydroxycarbonylC₂₋₆alkenyl, heteroC₁₋₆alkyl, heteroC₂₋₆alkenyl, C₅₋₁₂aryl, C₅₋₁₂arylC₁₋₆alkyl-, heteroaryl, heterocyclyl, C₁₋₆alkyloxy, and C₁₋₆alkylthio; and
   wherein each Z² is independently selected from the group comprising C₁₋₆alkyl, C₂₋₆alkenyl, hydroxycarbonylC₁₋₆alkyl, hydroxycarbonylC₂₋₆alkenyl, heteroC₁₋₆alkyl, heteroC₂₋₆alkenyl, C₅₋₁₂aryl, heteroaryl, and heterocyclyl,

Statement 8: The modulator according to any of statements 1 to 3 and 6 to 7, wherein said modulator is any of compounds 25 to 46 as selected from Table B. Yes/No in the 5^{th} and 6^{th} columns indicates whether there is, or respectively is not an inhibition of the synthetase (ST) and/or hydrolase (HD) activity.

TABLE B

| Compound | Molport | | | | |
|---|---|---|---|---|---|
| # | ID | ZINC ID | ST | HD | |
| 25 | MolPort- 000-645- 550 | ZINC00000285 3349 | No | Yes | 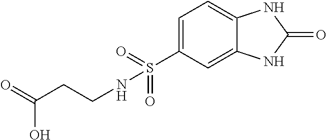 [SMILES: O=C(O)CCNS(=O)(=O)c1ccc2[nH]c(=O)[nH]c2c1] |
| 26 | MolPort- 009-323- 507 | ZINC00004011 9627 | No | Yes | 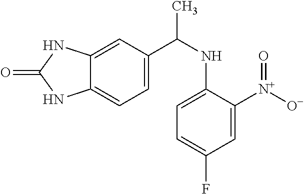 [SMILES: CC(Nc1ccc(F)cc1[N+](=O)[O-])c1ccc2[nH]c(=O)[nH]c2c1] |
| 27 | MolPort- 006-067- 386 | ZINC00000471 6673 | No | Yes | 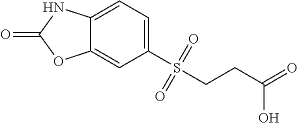 [SMILES: O=C(O)CCS(=O)(=O)c1ccc2[nH]c(=O)oc2c1] |
| 28 | MolPort- 038-405- 199 | ZINC00022000 6608 | No | Yes | 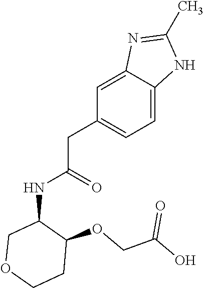 [SMILES: Cc1nc2cc(CC(=O)N[C@@H]3COCC[C@@H]3OCC(=O)O)ccc2[nH]1] |
| 29 | MolPort- 008-347- 407 | ZINC00003212 4075 | No | Yes | 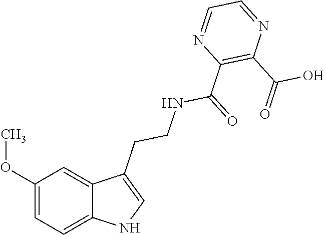 [SMILES: COc1ccc2[nH]cc(CCNC(=O)c3nccnc3C(=O)O)c2c1] |
| 30 | MolPort- 009-293- 972 | ZINC00002509 9308 | No | Yes | 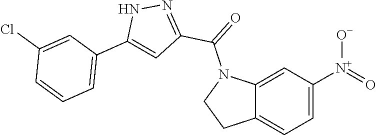 [SMILES: O=C(c1cc(-c2cccc(Cl)c2)[nH]n1)N1CCc2ccc([N+](=O)[O-])cc21] |
| 31 | MolPort- 001-895- 231 | ZINC00000653 4237 | No | Yes | 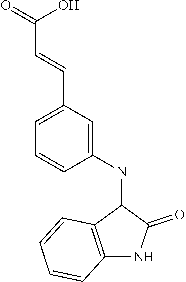 [SMILES: O=C(O)/C=C/c1cccc(NC2C(=O)Nc3ccccc32)c1] |
| 32 | MolPort- 035-871- 076 | ZINC00009611 7868 | No | Yes | 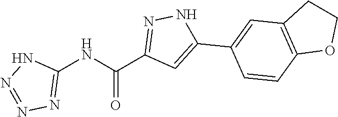 [SMILES: O=C(Nc1nnn[nH]1)c1cc(-c2ccc3c(c2)CCO3)[nH]n1] |
| 33 | MolPort- 002-267- 714 | ZINC00000111 5769 | No | Yes | 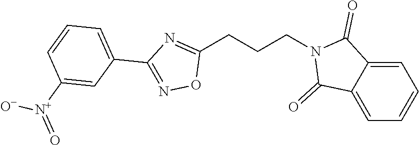 [SMILES: O=C1c2ccccc2C(=O)N1CCCc1nc(-c2cccc([N+](=O)[O-])c2)no1] |
| 34 | MolPort- 005-684- 722 | ZINC00001264 8085 | No | Yes | 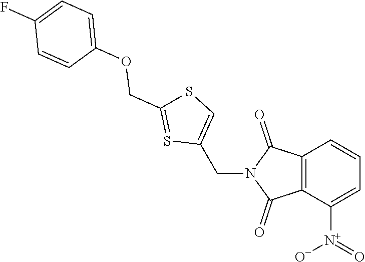 |
| 35 | MolPort- 045-943- 055 | ZINC00095297 4179 | no | yes | 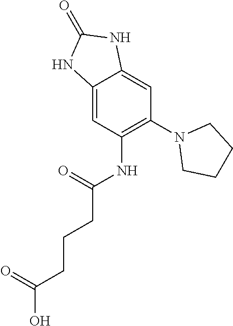 [SMILES: O=C(O)CCCC(=O)Nc1cc2[nH]c(=O)[nH]c2cc1N1CCCC1] |
| 36 | MolPort- 001-966- 650 | ZINC00000229 1004 | no | yes | 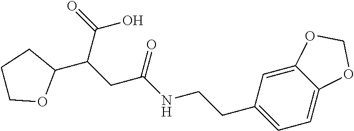 [SMILES: O=C(CC(C(=O)O)C1CCCO1)NCCc1ccc2c(c1)OCO2] |
| 37 | MolPort- 044-261- 510 | ZINC00001315 0353 | Yes | No | 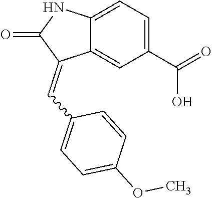 [SMILES: COc1ccc(C=C2C(=O)Nc3ccc(C(=O)O)cc32)cc1] |
| 38 | MolPort- 001-026- 283 | ZINC00001237 7200 | Yes | No | 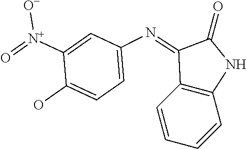 [SMILES: O=C1Nc2ccccc2/C1=N\c1ccc(O)c([N+](=O)[O-])c1] |
| 39 | MolPort- 001-620- 066 | ZINC00001237 7203 | Yes | No | 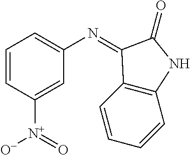 [SMILES: O=C1Nc2ccccc2/C1=N\c1cccc([N+](=O)[O-])c1] |
| 40 | MolPort- 002-954- 680 | ZINC00000057 5100 | Yes | No | 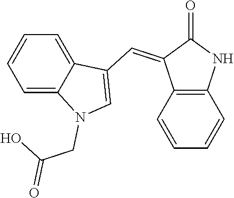 [SMILES: O=C(O)Cn1cc(/C=C2/C(=O)Nc3ccccc32)c2ccccc21] |
| 41 | MolPort- 000-419- 121 | ZINC00000082 3853 | Yes | Yes | 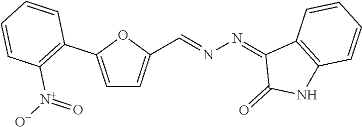 [SMILES: O=C1Nc2ccccc2/C1=N/N=C/c1ccc(-c2ccccc2[N+](=O)[O-])o1] |
| 42 | MolPort- 005-375- 663 | ZINC00001346 0182 | Yes | Yes | 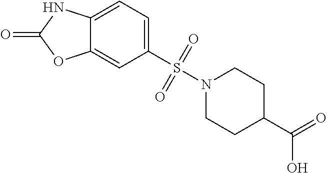 [SMILES: O=C(O)C1CCN(S(=O)(=O)c2ccc3[nH]c(=O)oc3c2)CC1] |
| 43 | MolPort- 020-098- 540 | ZINC00006938 4434 | Yes | Yes | 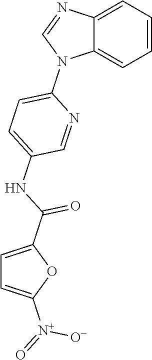 [SMILES: O=C(Nc1ccc(-n2cnc3ccccc32)nc1)c1ccc([N+](=O)[O-])o1] |
| 44 | MolPort- 028-811- 591 | ZINC00008987 4952 | Yes | Yes | 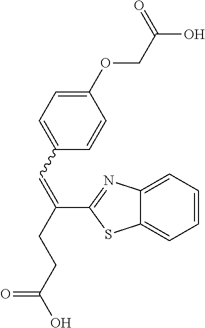 [SMILES: O=C(O)CCC(=Cc1ccc(OCC(=O)O)cc1)c1nc2ccccc2s1] |
| 45 | MolPort- 003-002- 903 | ZINC00001817 3319 | Yes | Yes | 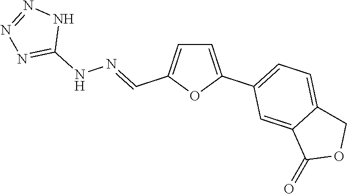 [SMILES: O=C1OCc2ccc(-c3ccc(/C=N/Nc4nnn[nH]4)o3)cc21] |
| 46 | MolPort- 019-773- 598 | ZINC00003391 9407 | Yes | Yes | 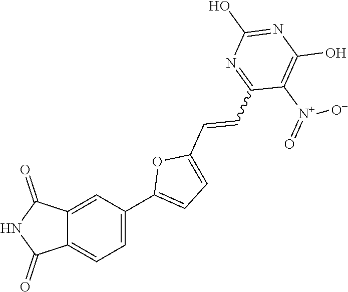 [SMILES: O=C1NC(=O)c2cc(-c3ccc(C=Cc4nc(O)nc(O)c4[N+](=O)[O-])o3)ccc21] |

Statement 9: Modulator of Rel hydrolase and/or synthetase activity, wherein said modulator is a compound of formula (II), or an isomer, preferably a stereo-isomer or a tautomer, a solvate, a salt, preferably a pharmaceutically acceptable salt, or a prodrug thereof,

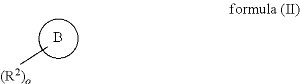

wherein o is an integer selected from 1, 2, 3, 4, 5, 6, or 7; and preferably selected from 1, 2, 3, 4, or 5, and preferably selected from 1, 2, 3, or 4;
wherein each R² is independently selected from halogen, ═S, ═O, nitro, or a group comprising hydroxyl, —SH, —NH₂, —C(O)OH, C₁₋₆alkyl, C₂₋₆alkenyl, haloC₁₋₆alkyl, C₃₋₁₈cycloalkyl, cycloalkenyl, heteroC₁₋₆alkyl, heteroC₂₋₆alkenyl, C₅₋₁₂aryl, heteroaryl, heterocyclyl, C₅₋₁₂arylC₁₋₆alkyl-, C₅₋₁₂arylC₂₋₆alkenyl-, C₅₋₁₂aryl-heteroC₁₋₆alkyl-, C₅₋₁₂aryl-heteroC₂₋₆alkenyl-, C₅₋₁₂aryl-heteroaryl-; C₅₋₁₂aryl-heterocyclyl-, heterocyclyl-C₁₋₆alkyl-; heterocyclyl-C₂₋₆alkenyl-, heterocyclyl-heteroC₁₋₆alkyl-, heterocyclyl-heteroC₂₋₆alkenyl-, heteroaryl-C₁₋₆alkyl-, heteroaryl-C₁₋₆alkenyl-, heteroaryl-heteroC₁₋₆alkyl-, heteroaryl-heteroC₂₋₆alkenyl-, C₁₋₆alkyloxy, haloC₁₋₆alkoxy, C₂₋₆alkenyloxy, C₅₋₁₂aryloxy; heteroaryloxy, heterocylyloxy, C₁₋₆alkylthio, C₂₋₆alkenylthio, C₅₋₁₂arylthio, heteroarylthio, heterocyclylthio, C_{5 42}aryl-C₁₋₆alkyloxy-, heteroaryl-C₁₋₆alkyloxy-, heterocyclyl-C₁₋₆alkyloxy-, C₅₋₁₂aryl-C₁₋₆alkylthio-, heteroaryl-C₁₋₆alkylthio-, heterocyclyl-C₁₋₆alkylthio-, hydroxycarbonylC₁₋₆alkyl-, hydroxycarbonylC₂₋₆alkenyl-, C₁₋₆alkyl-SO2-, C₂₋₆alkenyl-SO2-, heteroC₁₋₆alkyl-SO2-, heteroC₂₋₆alkenyl-SO2-, C₅₋₁₂aryl-SO2-, heteroaryl-SO2-, heterocyclyl-SO2-, heteroaryl-C₁₋₆alkyl-SO2-; heteroaryl-C₂₋₆alkenyl-SO2-, heteroaryl-heteroC₁₋₆alkyl-SO2-, heteroaryl-heteroC₂₋₆alkenyl-SO2- and heteroaryl-NH—SO2-,
   and wherein each of said groups can be unsubstituted or substituted with one or more substituents each independently selected from the group comprising halogen, nitro, oxo, C₁₋₆alkyloxy, —C(O)OH, —NH₂, hydroxycarbonylC₁₋₆alkyl, hydroxycarbonylC₂₋₆alkenyl, hydroxyl, C₁₋₆alkyl, C₂₋₆alkenyl, heteroC₁₋₆alkyl, heteroC₂₋₆alkenyl ═S, —SH, C₅₋₁₂aryl, heteroaryl, heterocyclyl.
wherein cycle B is selected from the group represented by formula (IIa);

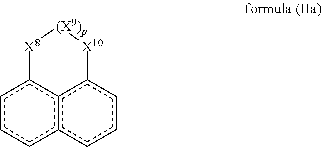

wherein the dotted line represents an optional double bond;
wherein p is an integer selected from 0 or 1;
wherein each of X⁸, X⁹, and X¹⁰ is independently selected from NH, N—OH, S, O, C═O, C═S, C(Z³)₂, and N(Z⁴), and
   wherein each Z³ is independently selected from the group comprising hydrogen, halogen, nitro, hydroxyl, —SH, —NH₂, —C(O)OH, C₁₋₆alkyl, C₂₋₆alkenyl, hydroxycarbonylC₁₋₆alkyl, hydroxycarbonylC₂₋₆alkenyl, heteroC₁₋₆alkyl, heteroC₂₋₆alkenyl, C₅₋₁₂aryl, heteroaryl, heterocyclyl, and C₁₋₆alkyloxy, and
   wherein each Z⁴ is independently selected from the group comprising C₁₋₆alkyl, C₂₋₆alkenyl, hydroxycarbonylC₁₋₆alkyl, hydroxycarbonylC₂₋₆alkenyl, heteroC₁₋₆alkyl, heteroC₂₋₆alkenyl, C₅₋₁₂aryl, heteroaryl, heterocyclyl, and C₁₋₆alkyloxy;
   and preferably with the proviso that when p is 1 and X⁹ is N—OH then X⁸ and X¹⁰ are C═O, and/or
   preferably with the proviso that when p is 0 and X⁸ is NH, then X¹⁰ is C═O, or when p is 0 and X⁸ is C═O, then X¹⁰ is NH.

Statement 10: The modulator according to statement 9, wherein said modulator is a compound of formula (II),

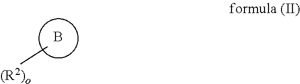

wherein o is an integer selected from 1, 2, 3 or 4;
wherein each R² is independently selected from halogen, ═O, nitro, or a group comprising hydroxyl, —C(O)OH, C₁₋₆alkyl, heteroC₁₋₆alkyl, C₅₋₁₂aryl, heteroaryl, C₅₋₁₂arylC₁₋₆alkyl-, C₅₋₁₂aryl-heteroC₁₋₆alkyl-, C₅₋₁₂aryl-heteroaryl-; heteroaryl-C₁₋₆alkyl-, heteroaryl-heteroC₁₋₆alkyl-, C₁₋₆alkyloxy, C₅₋₁₂aryloxy; heteroaryloxy, C₅₋₁₂aryl-C₁₋₆alkyloxy-, heteroaryl-C₁₋₆alkyloxy-, hydroxycarbonylC₁₋₆alkyl-, C₁₋₆alkyl-SO2-, heteroC₁₋₆alkyl-SO2-, C₅₋₁₂aryl-SO2-, heteroaryl-SO2-, heteroaryl-C₁₋₆alkyl-SO2-; heteroaryl-heteroC₁₋₆alkyl-SO2-, and heteroaryl-NH—SO2,
   and wherein each of said groups can be unsubstituted or substituted with one or more substituents each independently selected from the group comprising halogen, nitro, ═O, C₁₋₆alkyloxy, —C(O)OH, C₁₋₆alkyl, heteroC₁₋₆alkyl,
wherein cycle B is selected from the group represented by formula (IIa)

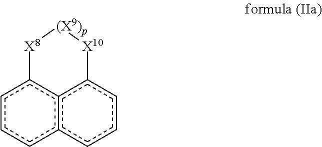

wherein the dotted line represents an optional double bond;
wherein p is an integer selected from 0 or 1;
wherein each of X⁸, X⁹, and X¹⁰ is independently selected from NH, N—OH, C═O, and C(Z³)₂, and N(Z⁴), and wherein each Z³ is independently selected from the group comprising hydrogen, halogen, nitro, hydroxyl, —C(O)OH, C₁₋₆alkyl, C₂₋₆alkenyl, and C₁₋₆alkyloxy; wherein each Z⁴ is independently selected from the group comprising C₁₋₆alkyl, C₂₋₆alkenyl, hydroxycarbonylC₁₋₆alkyl, hydroxycarbonylC₂₋₆alkenyl, heteroC₁₋₆alkyl, heteroC₂₋₆alkenyl, C₅₋₁₂aryl, heteroaryl, heterocyclyl, and C₁₋₆alkyloxy,
and preferably with the proviso that when p is 1 and X⁹ is N—OH, then X⁸ and X¹⁰ are C═O, and/or
preferably with the proviso that when p is 0 and X⁸ is NH, then X¹⁰ is C═O, or when p is 0 and X⁸ is C═O, then X¹⁰ is NH.

Statement 11: The modulator according any of statements 9 or 10, wherein said modulator is a compound of formula (II),

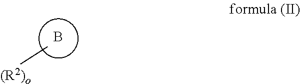

wherein o is an integer selected from 1, 2, 3 or 4; and preferably selected from 1, 2 or 3;
wherein each R² is independently selected from halogen, ═O, nitro, or a group comprising C₁₋₆alkyl, heteroC₁₋₆alkyl, C₅₋₁₂aryl, heteroaryl, C₁₋₆alkyl-SO2-, heteroC₁₋₆alkyl-SO2-, heteroaryl-heteroC₁₋₆alkyl-SO2-, heteroaryl-heteroC₁₋₆alkyl, and heteroaryl-NH-SO2, and wherein each of said groups can be unsubstituted or substituted with one or more substituents each independently selected from the group comprising halogen, nitro, ═O, C(O)OH, heteroC₁₋₆alkyl and C₁₋₆alkyl; and
wherein cycle B is selected from the group represented by formula (IIa);

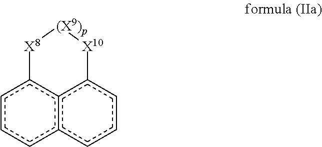

wherein the dotted line represents an optional double bond;
wherein p is 0; and
wherein each of X⁸ and X¹⁰ is independently selected from NH, C═O, C(Z³)₂ and N(Z⁴), and wherein each Z³ is independently selected from the group comprising hydrogen, nitro, hydroxyl, and —C(O)OH and wherein each Z⁴ is independently selected from the group comprising C₁₋₆alkyl, C₂₋₆alkenyl, hydroxycarbonylC₁₋₆alkyl, hydroxycarbonylC _{2 -6}alkenyl, heteroC₁₋₆alkyl, heteroC₂₋₆alkenyl, C₅₋₁₂aryl, heteroaryl, heterocyclyl, and C₁₋₆alkyloxy; and preferably wherein X⁸ is NH and X^{th} is C═O, or preferably wherein X⁸ is C═O and X¹⁰ is NH.

Statement 12: The modulator according to any of statements 9 to 11, wherein said modulator is any of compounds 47-49 as selected from Table C. Yes/No in the 5^{th} and 6^{th} columns indicates whether there is, or respectively is not an inhibition of the synthetase (ST) and/or hydrolase (HD) activity.

TABLE C

| Compound | Molport | | | | |
|---|---|---|---|---|---|
| # | ID | ZINC ID | ST | HD | |
| 47 | MolPort- 000-646- 902 | ZINC00000205 5771 | Yes | Yes | 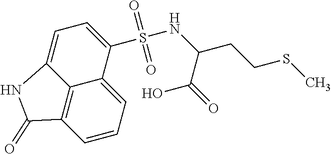 [SMILES: CSCCC(NS(=O)(=O)c1ccc2c3c(cccc13)C(=O)N2)C(=O)O] |
| 48 | MolPort- 000-628- 281 | ZINC00000082 8930 | Yes | Yes | 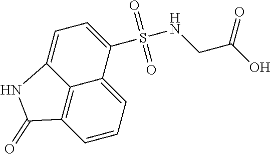 [SMILES: O=C(O)CNS(=O)(=O)c1ccc2c3c(cccc13)C(=O)N2] |
| 49 | MolPort- 023-244- 932 | ZINC00007910 3031 | Yes | Yes | 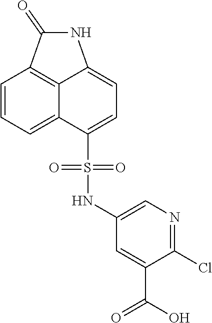 [SMILES: O=C(O)c1cc(NS(=O)(=O)c2ccc3c4c(cccc24)C(=O)N3)cnc1Cl] |

Statement 13: The modulator according to statement 9 or 10, wherein said modulator is a compound of formula (II),

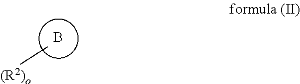

wherein o is an integer selected from 1, 2, 3 or 4;
wherein each R² is independently selected from halogen, ═O, nitro, or a group comprising hydroxyl, C₁₋₆alkyl, heteroC₁₋₆alkyl, C₅₋₁₂aryl, heteroaryl, C₁₋₆alkyl-SO2-, heteroC₁₋₆alkyl-SO2-, C₅₋₁₂aryl-SO2-, heteroaryl-SO2-, heteroaryl-C₁₋₆alkyl-SO2-; and heteroaryl-heteroC₁₋₆alkyl-SO2-, and wherein each of said groups can be unsubstituted or substituted with one or more substituents each independently selected from the group comprising halogen, nitro, ═O, C₁₋₆alkyloxy, —C(O)OH, C₁₋₆alkyl, and heteroC₁₋₆alkyl;
wherein cycle B is a group represented by formula (IIa),

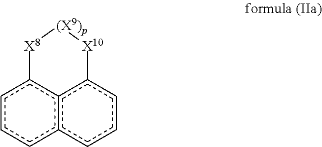

wherein the dotted line represents an optional double bond;
wherein p is 1; and
wherein each of X⁸, X⁹, and X¹⁰ is independently selected from NH, N—OH, C═O, C(Z³)₂ and N(Z⁴), wherein each Z³ is independently selected from the group comprising hydrogen, halogen, nitro, hydroxyl, —C(O)OH, C₁₋₆alkyl, C₂₋₆alkenyl, and C₁₋₆alkyloxy wherein each Z⁴ is independently selected from the group comprising C₁₋₆alkyl, C₂₋₆alkenyl, hydroxycarbonylC₁₋₆alkyl, hydroxycarbonylC₂₋₆alkenyl, heteroC₁₋₆alkyl, heteroC₂₋₆alkenyl, C₅₋₁₂aryl, heteroaryl, heterocyclyl, and C₁₋₆alkyloxy; and preferably with the proviso that when X⁹ is N—OH or NH, X⁸ and X¹⁰ are C═O.

Statement 14: The modulator according to any of statements 9, 10, and 13, wherein said modulator is any of compounds 50-51 as selected from Table D. Yes/No in the 5^{th} and 6^{th} columns indicates whether there is, or respectively is not an inhibition of the synthetase (ST) and/or hydrolase (HD) activity.

TABLE D

| Compound # | Molport ID | ZINC ID | ST | HD | |
|---|---|---|---|---|---|
| 50 | MolPort- 000-564-789 | ZINC000000100308 | Yes | No | 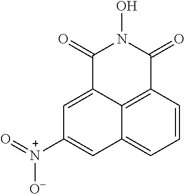 [SMILES: O=C1c2cccc3cc([N+](=O)[O-])cc(c23)C(=O)N1O] |
| 51 | MolPort- 008-346-372 | ZINC000035686171 | Yes | Yes | 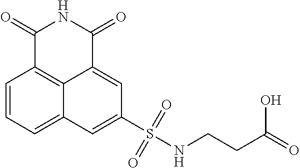 [SMILES: O=C(O)CCNS(=O)(=O)c1cc2c3c(cccc3c1)C(=O)NC2=O] |

Statement 15: Modulator of Rel hydrolase and/or synthetase activity, wherein said modulator is a compound of formula (III), or an isomer, preferably a stereo-isomer or a tautomer, a solvate, a salt, preferably a pharmaceutically acceptable salt, or a prodrug thereof,

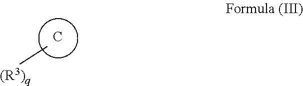

wherein q is an integer selected from 1, 2, 3, 4, 5, or 6; and
wherein each R³ is independently selected from halogen, ═S, ═O, nitro, or a group comprising hydroxyl, —SH, —NH₂, —C(O)OH, C₁₋₆alkyl, C₂₋₆alkenyl, haloC₁₋₆alkyl, hydroxycarbonylC₁₋₆alkyl, hydroxycarbonylC₂₋₆alkenyl, C₃₋₁₈cycloalkyl, cycloalkenyl, heteroC₁₋₆alkyl, heteroC₂₋₆alkenyl, C₅₋₁₂aryl, heteroaryl, heterocyclyl, C₁₋₆alkyloxy, C₁₋₆alkylthio, heterocyclyl-C₁₋₆alkyl-, heterocyclyl-heteroC₁₋₆alkyl-, heterocyclyl-heteroaryl, C₅₋₁₂aryl-C₁₋₆alkyl-, C₅₋₁₂arylC₂₋₆alkenyl-, C₅₋₁₂aryl-heteroC₁₋₆alkyl-; C₅₋₁₂aryl-heteroC2-6alkenyl-, C₅₋₁₂aryl-heteroaryl-; C₅₋₁₂aryl-heterocyclyl-, heterocyclyl-C₁₋₆alkyl-; heterocyclyl-C₂₋₆alkenyl-, heterocyclyl-heteroC₂₋₆alkenyl-, heteroarylC₁₋₆alkyl-, heteroarylC₁₋₆alkenyl-, heteroaryl-heteroC₁₋₆alkyl-, heteroaryl-heteroC₂₋₆alkenyl-, C₁₋₆alkyloxy-, C₂₋₆alkenyloxy-, C₅₋₁₂aryloxy-; heteroaryloxy-, heterocylyloxy-, C₁₋₆alkylthio-, C₂₋₆alkenylthio-, C₅₋₁₂arylthio-, heteroarylthio-, heterocyclylthio-, C₅₋₁₂aryl-C₁₋₆alkyloxy-, heteroaryl-C₁₋₆alkyloxy-, heterocyclyl-C₁₋₆alkyloxy-, C₅₋₁₂aryl-C₁₋₆alkylthio-, heteroaryl-C₁₋₆alkylthio-, heterocyclyl-C₁₋₆alkylthio-, C₁₋₆alkyl-SO2-, C₂₋₆alkenyl-SO2-, heteroC₁₋₆alkyl-SO2-, heteroC₂₋₆alkenyl-SO2-, C₅₋₁₂aryl-SO2-, heteroaryl-SO2-, heterocyclyl-SO2-, heteroaryl-heteroC₁₋₆alkyl-heteroaryl-, Heteroaryl-heteroC₂₋₆alkenyl-heteroaryl-, heteroaryl-C₁₋₆alkyl-heteroaryl-, Heteroaryl-C₂₋₆alkenyl-heteroaryl-, C₅₋₁₂Aryl-heteroaryl-C₁₋₆alkyl-, C₅₋₁₂aryl-heteroaryl-C₂₋₆alkenyl-, C₅₋₁₂aryl-heteroaryl-heteroC₁₋₆alkyl-, C₅₋₁₂aryl-heteroaryl-heteroC₂₋₆alkenyl-, C₅₋₁₂aryl-C₂₋₆alkenyl-, C₁₋₆Alkyloxy-C₅₋₁₂aryl-C₁₋₆alkyl-, Alkyloxy-heteroaryl-C₁₋₆alkyl-, C₁₋₆Alkyloxy-heteroaryl-C₂₋₆alkenyl-, C₁₋₆Alkyloxy-heterocyclyl-C₁₋₆alkyl-, C₁₋₆Alkyloxy-heterocyclyl-C₂₋₆alkenyl-, C₁₋₆Alkyloxy-heterocyclyl-heteroC₁₋₆alkyl-, C₁₋₆Alkyloxy-heterocyclyl-heteroC₂₋₆alkenyl-, C₅₋₁₂Aryl-heteroC₁₋₆alkyl-heteroaryl-, C₅₋₁₂aryl-heteroC₂₋₆alkenyl-heteroaryl-, C₅₋₁₂Aryl-heteroC₁₋₆alkyl-heteroaryl-C₁₋₆alkyl-, C₅₋₁₂Aryl-heteroC₂₋₆alkenyl-heteroaryl-C₁₋₆alkyl-, C₅₋₁₂aryl-heteroC₁₋₆alkyl-heterocyclyl-, C₅₋₁₂aryl-heteroaryl-heterocyclyl-; C₅₋₁₂Aryl-C₁₋₆alkyl-heterocyclyl C₅₋₁₂Aryl-C₂₋₆alkenyl-heterocyclyl, C₂₋₆alkenyl-C₅₋₁₂aryl-heteroC₂₋₆alkenyl-, C₅₋₁₂ArylC₁₋₆Alkyl-heterocyclyl-C₁₋₆alkyl-, C₅₋₁₂Aryl-C₁₋₆Alkyl-heterocyclyl-C₂₋₆alkenyl-, C₅₋₁₂Aryl-C₁₋₆Alkyl-heterocyclyl-heteroC₁₋₆alkyl-, C₅₋₁₂Aryl-C₁₋₆Alkyl-heterocyclyl-heteroC₂₋₆alkenyl-, C₅₋₁₂Aryl-C₂₋₆alkenyl-heterocyclyl-C₁₋₆alkyloxy-, C₅₋₁₂Aryl-C₁₋₆alkyl-heterocyclyl-C₁₋₆alkyloxy-, C₅₋₁₂Aryl-C₂₋₆alkenyl-heteroaryl-C₁₋₆alkyloxy-, C₅₋₁₂Aryl-C₁₋₆alkyl-heteroaryl-C₁₋₆alkyloxy-, C₅₋₁₂Aryl-C₁₋₆alkyl-heterocyclyl-SO2-, C₅₋₁₂aryl-C₂₋₆alkenyl-heterocyclyl-SO2-, heteroaryl-C₁₋₆alkyl-heterocyclyl-SO2-, heteroaryl-C₂₋₆alkenyl-heterocyclyl-SO2-; C₅₋₁₂aryl-amino, and C₅₋₁₂aryl-NH— and wherein each of said groups can be unsubstituted or substituted with one or more substituents each independently selected from the group comprising halogen, nitro, oxo, C₁₋₆alkyloxy, —C(O)OH, —NH₂, hydroxycarbonylC₁₋₆alkyl, hydroxycarbonylC₂₋₆alkenyl, hydroxyl, C₁₋₆alkyl, C₂₋₆alkenyl, haloC₁₋₆alkyl, haloC₂₋₆alkenyl, heteroC₁₋₆alkyl, heteroC₂₋₆alkenyl ═S, —SH, C₅₋₁₂aryl, heteroaryl, heterocyclyl; C₅₋₁₂aryl-C₁₋₆alkyl-; C₅₋₁₂aryl-C₂₋₆alkenyl-; C₅₋₁₂arylheteroC₁₋₆alkyl-; C₅₋₁₂arylheteroC₂₋₆alkenyl-; and heterocyclyl-C₁₋₆alkyl-; and
wherein cycle C is selected from the group represented by formula (IIIa);

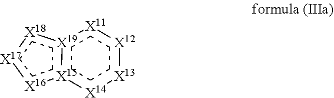

wherein the dotted line represents an optional double bond;
wherein each of X¹⁵ and X¹⁹ is independently selected from N, C, and CH,
wherein each of X¹¹, X¹², X¹³, X¹⁴, X¹⁶, X¹⁷, and X¹⁸ is independently selected from the group comprising N, NH, S, O, C═O, C═S, CH, C(Z⁵)₂ and N(Z⁶), and
   wherein each Z⁵ is independently selected from the group comprising hydrogen, halogen, nitro, hydroxyl, —SH, —NH₂, —C(O)OH, C₁₋₆alkyl, heteroC₁₋₆alkyl, C₂₋₆alkenyl, heteroC₂₋₆alkenyl, C₅₋₁₂aryl, heteroaryl, heterocyclyl, C₁₋₆alkyloxy, C₁₋₆alkylthio heterocyclyl-C₁₋₆alkyl-, heterocyclyl-C₂₋₆alkenyl-, heterocyclyl-heteroC₁₋₆alkyl-, heterocyclyl-heteroC₂₋₆alkenyl-, C₅₋₁₂arylC₁₋₆alkyl-, C₅₋₁₂aryl-C₂₋₆alkenyl-, C₅₋₁₂aryl-heteroC₁₋₆alkyl-; C₅₋₁₂aryl-heteroC₂₋₆alkenyl-, heteroarylC₁₋₆alkyl-, heteroaryl-C₁₋₆alkenyl-, heteroaryl-hetero C₁₋₆alkyl-, heteroaryl-heteroC₂₋₆alkenyl-, heteroaryl-C₁₋₆alkyl-heteroaryl-, Heteroaryl-heteroC₁₋₆alkyl-heteroaryl-, C₅₋₁₂Aryl-heteroaryl-C₁₋₆alkyl-, C₅₋₁₂Aryl-heteroaryl-C₂₋₆alkenyl, C₅₋₁₂aryl-heteroaryl-heteroC₁₋₆alkyl-, C₅₋₁₂Aryl-heteroaryl-heteroC₂₋₆alkenyl, C₅₋₁₂aryl-heteroC₁₋₆alkyl-heterocyclyl-, hydroxycarbonylC₁₋₆alkyl, and hydroxycarbonylC₂₋₆alkenyl; and
   wherein each Z⁶ is independently selected from the group comprising C₁₋₆alkyl, C₂₋₆alkenyl, halo C₁₋₆alkyl, hydroxycarbonylC₁₋₆alkyl, hydroxycarbonylC₂₋₆alkenyl, heteroC₁₋₆alkyl, heteroC₂₋₆alkenyl, C₅₋₁₂aryl, heteroaryl, heterocyclyl, C₁₋₆alkyloxy, C₅₋₁₂arylC₁₋₆alkyl-, C₅₋₁₂arylC₂₋₆alkenyl-, C₅₋₁₂arylheteroC₁₋₆alkyl-, C₅₋₁₂arylheteroC₂₋₆alkenyl-, heteroarylC₁₋₆-alkyl-, heteroaryl-C₁₋₆alkenyl-, heterocyclyl-C₁₋₆alkyl-, heterocyclyl-C₂₋₆alkenyl-, heterocyclyl-heteroC₁₋₆alkyl-; heterocyclyl-heteroC₂₋₆alkenyl-, heteroaryl-heteroC₁₋₆alkyl-, heteroaryl-heteroC₂₋₆alkenyl-, heteroaryl-C₁₋₆alkyl-heteroaryl-, Heteroaryl-heteroC₁₋₆alkyl-heteroaryl-, C₅₋₁₂Aryl-heteroaryl-C₁₋₆alkyl-, C₅₋₁₂Aryl-heteroaryl-C₂₋₆alkenyl, C₅₋₁₂aryl-heteroaryl-heteroC₁₋₆alkyl-, and C₅₋₁₂Aryl-heteroaryl-heteroC₂₋₆alkenyl.

Statement 16: Modulator according to statement 15, wherein said modulator is a compound of formula (III),

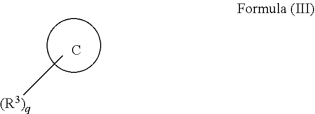

wherein q is an integer selected from 1, 2, 3 or 4; and
wherein each R³ is independently selected from halogen, ═O, nitro, or a group comprising hydroxyl, —C(O)OH, C₁₋₆alkyl, heteroC₁₋₆alkyl, C₁₋₆alkylthio, C₅₋₁₂aryl, heteroaryl, heterocyclyl, heterocyclyl-C₁₋₆alkyl-, heterocyclyl-heteroC₁₋₆alkyl-, heterocyclyl-heteroaryl, C₅₋₁₂aryl-C₁₋₆alkyl-, C₅₋₁₂aryl-heteroC₁₋₆alkyl-, C₅₋₁₂aryl-C₁₋₆alkyl-heterocyclyl-, C₅₋₁₂aryl-heteroC₁₋₆alkyl-heterocyclyl-, C₅₋₁₂aryl-heteroaryl-heterocyclyl-; C₅₋₁₂aryl-amino, and C₅₋₁₂aryl-NH— and wherein each of said groups can be unsubstituted or substituted with one or more substituents each independently selected from the group comprising oxo, nitro, —C(O)OH, hydroxyl, C₁₋₆alkyl, heteroC₁₋₆alkyl, heterocyclyl; heterocyclyl-C₁₋₆alkyl-, and hydroxycarbonyl C₁₋₆alkyl; and
wherein cycle C is selected from the group represented by formula (IIIa);

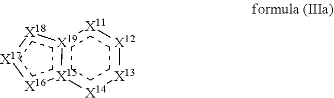

wherein the dotted line represents an optional double bond;
wherein each of X¹⁵ and X¹⁹ is independently selected from N, C, and CH,
wherein each of X¹¹, X¹², X¹³, X¹⁴, X¹⁶, X¹⁷, and X¹⁸ is independently selected from the group comprising N, NH, S, O, C═O, C═S, CH, C(Z⁵)₂ and N(Z⁶), and
   wherein each Z⁵ is independently selected from the group comprising hydrogen, halogen, nitro, hydroxyl, —SH, —NH₂, —C(O)OH, C₁₋₆alkyl, heteroC₁₋₆alkyl, C₂₋₆alkenyl, heteroC₂₋₆alkenyl, C₅₋₁₂aryl, heteroaryl, heterocyclyl, C₁₋₆alkyloxy, C₁₋₆alkylthio, heterocyclyl-C₁₋₆alkyl-, heterocyclyl-heteroC₁₋₆alkyl-, C₅₋₁₂aryl-heteroC₁₋₆alkyl-; C₅₋₁₂aryl-heteroC₁₋₆alkyl-heterocyclyl-, hydroxycarbonylC₁₋₆alkyl, and hydroxycarbonylC₂₋₆alkenyl; and
   wherein each Z⁶ is independently selected from the group comprising C₁₋₆alkyl, C₂₋₆alkenyl, hydroxycarbonylC₁₋₆alkyl, hydroxycarbonylC₂₋₆alkenyl, heteroC₁₋₆alkyl, heteroC₂₋₆alkenyl, C₅₋₁₂aryl, heteroaryl, heterocyclyl, and C₁₋₆alkyloxy.

Statement 17: Modulator according to statement 15 or 16, wherein said modulator is a compound of formula (III),

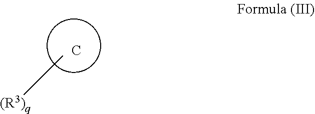

wherein q is an integer selected from 1, 2, 3 or 4; and
wherein each R³ is independently selected from ═O, or a group comprising hydroxyl, —C(O)OH, C₁₋₆alkyl, heteroC₁₋₆alkyl, C₁₋₆alkylthio, heterocyclyl, heterocyclyl-C₁₋₆alkyl-, heterocyclyl-heteroC₁₋₆alkyl-, heterocyclyl-heteroaryl, C₅₋₁₂aryl-C₁₋₆alkyl-, C₅₋₁₂aryl-heteroC₁₋₆alkyl-, C₅₋₁₂aryl-C₁₋₆alkyl-heterocyclyl-, C₅₋₁₂aryl-heteroC₁₋₆alkyl-heterocyclyl-, C₅₋₁₂aryl-heteroaryl-heterocyclyl-; C₅₋₁₂aryl-amino, and C₅₋₁₂aryl-NH— and wherein each of said groups can be unsubstituted or substituted with one or more substituents each independently selected from the group comprising oxo, nitro, —C(O)OH, hydroxyl, C₁₋₆alkyl, heteroC₁₋₆alkyl, heterocyclyl; heterocyclyl-C₁₋₆alkyl- and hydroxycarbonyl C₁₋₆alkyl;
wherein cycle C is selected from the group represented by formula (IIIa);

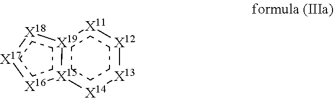

wherein the dotted line represents an optional double bond;
wherein each of X¹⁵ and X¹⁹ is independently selected from the group comprising N, C, and CH,
wherein each of X¹¹, X¹², X¹³, X¹⁴, X¹⁶, X¹⁷, and X¹⁸ is independently selected from the group comprising N, NH, S, O, C═O, CH, C(Z⁵)₂ and N(Z⁶), and
   wherein each Z⁵ is independently selected from a group comprising hydrogen, halogen, nitro, hydroxyl, —SH, —NH₂, —C(O)OH, C₁₋₆alkyl, C₂₋₆alkenyl, hydroxycarbonylC₁₋₆alkyl, hydroxycarbonylC₂₋₆alkenyl, heteroC₁₋₆alkyl, heteroC₂₋₆alkenyl, C₅₋₁₂aryl, heteroaryl, heterocyclyl, C₁₋₆alkyloxy, and C₁₋₆alkylthio, and
   wherein each Z⁶ is independently selected from the group comprising C₁₋₆alkyl, C₂₋₆alkenyl, hydroxycarbonylC₁₋₆alkyl, hydroxycarbonylC₂₋₆alkenyl, heteroC₁₋₆alkyl, heteroC₂₋₆alkenyl, C₅₋₁₂aryl, heteroaryl, and heterocyclyl.

Statement 18: Modulator according to any of statement 15 to 17, wherein said modulator is a compound of formula (III), and wherein cycle C is selected from the group comprising

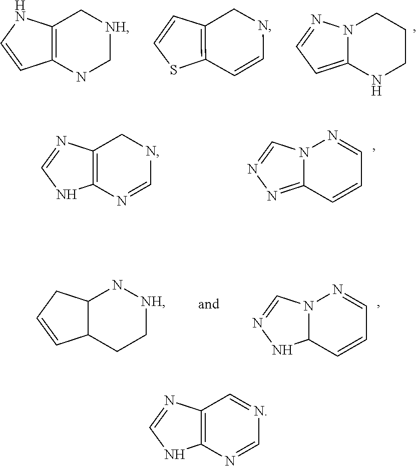

Statement 19: Modulator according to any of statements 15 to 18 wherein said modulator is any of compounds 52-56 as selected from Table E. Yes/No in the 5th and 6th columns indicates whether there is, or respectively is not an inhibition of the synthetase (ST) and/or hydrolase (HD) activity.

TABLE E

| Compound | Molport | | | | |
|---|---|---|---|---|---|
| # | ID | ZINC ID | ST | HD | |
| 52 | MolPort- 004-243- 877 | ZINC00000319 7434 | No | Yes | 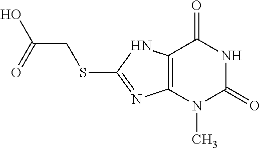 [SMILES: Cn1c(=O)[nH]c(=O)c2[nH]c(SCC(=O)O)nc21] |
| 53 | MolPort- 039-296- 313 | ZINC00029975 2468 | No | Yes | 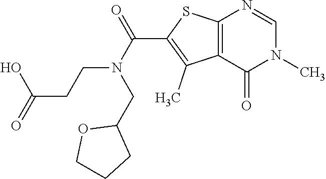 [SMILES: Cc1c(C(=O)N(CCC(=O)O)CC2CCCO2)sc2ncn(C)c(=O)c12] |
| 54 | MolPort- 042-686- 924 | ZINC00058365 0643 | No | Yes | 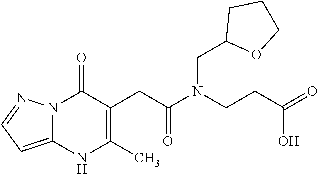 [SMILES: Cc1[nH]c2ccnn2c(=O)c1CC(=O)N(CCC(=O)O)CC1CCCO1] |
| 55 | MolPort- 008-347- 743 | ZINC00003212 4528 | No | Yes | 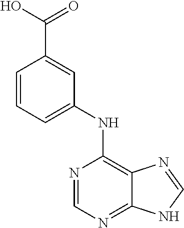 [SMILES: O=C(O)c1cccc(Nc2ncnc3[nH]cnc23)c1] |
| 56 | MolPort- 007-661- 717 | ZINC00002035 5818 | No | Yes | 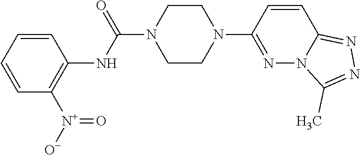 [SMILES: Cc1nnc2ccc(N3CCN(C(=O)Nc4ccccc4[N+](=O)[O-])CC3)nn12] |

Statement 20: Modulator of Rel hydrolase and/or synthetase activity, wherein said modulator is a compound of formula (IV),

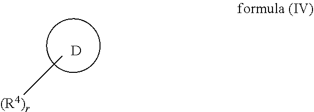

wherein cycle D is selected from the group heteroaryl, C₅₋₁₂aryl, heterocyclyl, and C₃₋₁₈cycloalkyl;
wherein r is an integer selected from 1, 2, 3, 4, 5 or 6; and preferably selected from 1, 2, 3 or 4; and
wherein each R⁴ is independently selected from halogen, nitro, or a group comprising hydroxyl, —NH₂, —C(O)OH, C₁₋₆alkyl, C₂₋₆alkenyl, haloC₁₋₆alkyl, C₃₋₁₈cycloalkyl, cycloalkenyl, heteroC₁₋₆alkyl, heteroC₂₋₆alkenyl, C₅₋₁₂aryl, heteroaryl, heterocyclyl, C₅₋₁₂arylC₁₋₆alkyl-, C₅₋₁₂arylC₂₋₆alkenyl-, C₅₋₁₂aryl-heteroC₁₋₆alkyl-, C₅₋₁₂aryl-heteroC₂₋₆alkenyl-, C₅₋₁₂aryl-heteroaryl-; C₅₋₁₂aryl-heterocyclyl-, C₁₋₆alkyl-heteroaryl-, C₂₋₆alkenyl-heteroaryl-, heteroC₁₋₆alkyl-heteroaryl-, heteroC₁₋₆alkyl-heterocyclyl, heteroC₂₋₆alkenyl-heteroaryl-, heteroC₂₋₆alkenyl-heterocyclyl-, heterocyclyl-C₁₋₆alkyl-; heterocyclyl-C₂₋₆alkenyl-, heterocyclyl-heteroC₁₋₆alkyl-, heterocyclyl-heteroC₂₋₆alkenyl-, heterocyclyl-heterocyclyl-, heteroaryl-C₁₋₆alkyl-, heteroaryl-C₁₋₆alkenyl-, heteroaryl-heteroC₁₋₆alkyl-, heteroaryl-heteroC₂₋₆alkenyl-, C₁₋₆alkyloxy, haloC₁₋₆alkoxy, C₂₋₆alkenyloxy, C₅₋₁₂aryloxy; heteroaryloxy, heterocylyloxy, C₁₋₆alkylthio, C₂₋₆alkenylthio, C₅₋₁₂arylthio, heteroarylthio, heterocyclylthio, C₅₋₁₂aryl-C₁₋₆alkyloxy-, heteroaryl-C₁₋₆alkyloxy-, heterocyclyl-C₁₋₆alkyloxy-, C₅₋₁₂aryl-C₁₋₆alkylthio-, heteroaryl-C₁₋₆alkylthio-, heterocyclyl-C₁₋₆alkylthio-, hydroxycarbonylC₁₋₆alkyl, hydroxycarbonylC₂₋₆alkenyl, C₁₋₆alkyl-SO2-, C₂₋₆alkenyl-SO2-, heteroC₁₋₆alkyl-SO2-, heteroC₂₋₆alkenyl-SO2-, C₅₋₁₂aryl-SO2-, heteroaryl-SO2-, heterocyclyl-SO2-, heteroaryl-C₁₋₆alkyl-SO2-; heteroaryl-C₂₋₆alkenyl-SO2-, heteroaryl-heteroC₁₋₆alkyl-SO2-, heteroaryl-heteroC₂₋₆alkenyl-SO2-, heteroaryl-heteroC₁₋₆alkyl-heteroaryl-, heteroaryl-heteroC₂₋₆alkenyl-heteroaryl-, heteroaryl-C₁₋₆alkyl-heteroaryl-, heteroaryl-C₂₋₆alkenyl-heteroaryl-, C₅₋₁₂Aryl-heteroaryl-C₁₋₆alkyl-, C₅₋₁₂aryl-heteroaryl-C₂₋₆alkenyl-, C₅₋₁₂aryl-heteroaryl-heteroC₁₋₆alkyl-, C₅₋₁₂aryl-heteroaryl-heteroC₂₋₆alkenyl-, C₅₋₁₂aryl-heteroC₁₋₆alkyl-heteroaryl-; C₅₋₁₂aryl-heteroC₁₋₆alkyl-C₅₋₁₂aryl-; C₅₋₁₂aryl-heteroC₁₋₆alkyl-heteroaryl-heteroC₁₋₆alkyl-; aryl-heteroC₁₋₆alkyl-heteroaryl-heteroC₂₋₆alkenyl, heteroaryl-heterocylcyl-C₁₋₆alkyl, and C₅₋₁₂aryl-NH—, aryl-NH-heteroaryl-heteroalkenyl-, nitroC₅₋₁₂aryl-, nitroC₅₋₁₂aryl-NH—, nitroC₅₋₁₂aryl-NH-heteroaryl-heteroalkenyl-; and wherein each of said groups can be unsubstituted or substituted with one or more substituents each independently selected from the group comprising halogen, nitro, oxo, C₁₋₆alkyloxy, —C(O)OH, —NH₂, hydroxycarbonylC₁₋₆alkyl, hydroxycarbonylC₂₋₆alkenyl, hydroxyl, C₁₋₆alkyl, C₂₋₆alkenyl, heteroC₁₋₆alkyl, heteroC₂₋₆alkenyl ═S, —SH, C₅₋₁₂aryl, nitroC₅₋₁₂aryl-, nitroC₅₋₁₂aryl-NH, heteroaryl, and heterocyclyl.

Statement 21: The modulator according to statement 20,
wherein cycle D is selected from the group heteroaryl, C₅₋₁₂aryl, heterocyclyl, and C₃₋₁₈cycloalkyl;
wherein r is an integer selected from 1, 2, 3 or 4; and
wherein each R⁴ is independently selected from halogen, nitro, or a group comprising hydroxyl, —NH₂, —C(O)OH, C₁₋₆alkyl, C₂₋₆alkenyl, C₃₋₁₈cycloalkyl, cycloalkenyl, heteroC₁₋₆alkyl, heteroC₂₋₆alkenyl, C₅₋₁₂aryl, heteroaryl, heterocyclyl, C₅₋₁₂arylC₁₋₆alkyl-, C₅₋₁₂arylC₂₋₆alkenyl-, C₅₋₁₂aryl-heteroC₁₋₆alkyl-, C₅₋₁₂aryl-heteroC₂₋₆alkenyl-, C₅₋₁₂aryl-heteroaryl-; C₅₋₁₂aryl-heterocyclyl-, C₁₋₆alkyl-heteroaryl-, C₂₋₆alkenyl-heteroaryl-, heteroC₁₋₆alkyl-heteroaryl-, heteroC₁₋₆alkyl-heterocyclyl, heteroC₂₋₆alkenyl-heteroaryl-, heteroC₂₋₆alkenyl-heterocyclyl-, heterocyclyl-C₁₋₆alkyl-; heterocyclyl-C₂₋₆alkenyl-, heterocyclyl-heteroC₁₋₆alkyl-, heterocyclyl-heteroC₂₋₆alkenyl-, heterocyclyl-heterocyclyl-, heteroaryl-C₁₋₆alkyl-, heteroaryl-C₁₋₆alkenyl-, heteroaryl-heteroC₁₋₆alkyl-, heteroaryl-heteroC₂₋₆alkenyl-, C₁₋₆alkyloxy, C₂₋₆alkenyloxy, C₅₋₁₂aryloxy; C₂₋₆alkenylthio, C₅₋₁₂arylthio, heteroarylthio, heterocyclylthio, heteroaryl-C₁₋₆alkyloxy-, heterocyclyl-C₁₋₆alkyloxy-, C₅₋₁₂aryl-C₁₋₆alkylthio-, heteroaryl-C₁₋₆alkylthio-, heterocyclyl-C₁₋₆alkylthio-, hydroxycarbonylC₁₋₆alkyl, hydroxycarbonylC₂₋₆alkenyl, C₅₋₁₂aryl-SO2-, heteroaryl-SO2-, heterocyclyl-SO2-, heteroaryl-heteroC₁₋₆alkyl-heteroaryl-, heteroaryl-heteroC₂₋₆alkenyl-heteroaryl-, heteroaryl-C₁₋₆alkyl-heteroaryl-, heteroaryl-C₂₋₆alkenyl-heteroaryl-, C₅₋₁₂Aryl-heteroaryl-C₁₋₆alkyl-, C₅₋₁₂aryl-heteroaryl-C₂₋₆alkenyl-, C₅₋₁₂aryl-heteroaryl-heteroC₁₋₆alkyl-, C₅₋₁₂aryl-heteroaryl-heteroC₂₋₆alkenyl-, C₅₋₁₂aryl-heteroC₁₋₆alkyl-heteroaryl-; C₅₋₁₂aryl-heteroC₁₋₆alkyl-C₅₋₁₂aryl-; C₅₋₁₂aryl-heteroC₁₋₆alkyl-heteroaryl-heteroC₁₋₆alkyl-; aryl-heteroC₁₋₆alkyl-heteroaryl-heteroC₂₋₆alkenyl, heteroaryl-heterocylcyl-C₁₋₆alkyl, and C₅₋₁₂aryl-NH—; aryl-NH-heteroaryl-heteroalkenyl-, nitroC₅₋₁₂aryl-, nitroC₅₋₁₂aryl-NH—, nitroC₅₋₁₂aryl-NH-heteroaryl-heteroalkenyl-, and wherein each of said groups can be unsubstituted or substituted with one or more substituents each independently selected from the group comprising halogen, nitro, oxo, C₁₋₆alkyloxy, —C(O)OH, —NH₂, hydroxycarbonylC₁₋₆alkyl, hydroxycarbonylC₂₋₆alkenyl, hydroxyl, C₁₋₆alkyl, C₂₋₆alkenyl, heteroC₁₋₆alkyl, heteroC₂₋₆alkenyl ═S, —SH, C₅₋₁₂aryl, nitroC₅₋₁₂aryl-, nitroC₅₋₁₂aryl-NH, heteroaryl, and heterocyclyl.

Statement 22: The modulator according to statement 20 or 21, wherein said modulator is a compound of formula (IV),

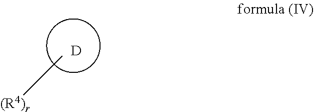

wherein cycle D is selected from the group comprising heteroaryl, C₅₋₁₂aryl, heterocyclyl, and C₃₋₁₈cycloalkyl; and preferably from the group comprising heteroaryl and C₅₋₁₂aryl,
wherein r is an integer selected from 1, 2, 3 or 4; and
wherein each R⁴ is independently selected from halogen, nitro, or a group comprising —NH₂, —C(O)OH, hydroxyl, C₁₋₆alkyl, heteroC₁₋₆alkyl, C₅₋₁₂aryl, heteroaryl, heterocyclyl, C₅₋₁₂arylC₁₋₆alkyl-, C₅₋₁₂aryl-heteroC₁₋₆alkyl-, C₅₋₁₂aryl-heteroC₂₋₆alkenyl-, C₅₋₁₂aryl-heteroaryl-; C₅₋₁₂aryl-heterocyclyl-, C₁₋₆alkyl-heteroaryl-, heteroC₁₋₆alkyl-heteroaryl-, heteroC₁₋₆alkyl-heterocyclyl, heterocyclyl-C₁₋₆alkyl-; heterocyclyl-heteroC₁₋₆alkyl-, heterocyclyl-heterocyclyl-, heteroaryl-C₁₋₆alkyl-, heteroaryl-heteroC₁₋₆alkyl-, C₁₋₆alkyloxy, C₁₋₆alkylthio C₅₋₁₂arylthio, heterocyclylthio, C₅₋₁₂aryl-C₁₋₆alkyloxy-, heteroaryl-C₁₋₆alkyloxy-, heterocyclyl-C₁₋₆alkyloxy-, Heteroaryl-heteroC₁₋₆alkyl-heteroaryl-, heteroaryl-C₁₋₆alkyl-heteroaryl-, C₅₋₁₂Aryl-heteroaryl-C₁₋₆alkyl-,C₅₋₁₂aryl-heteroaryl-heteroC₁₋₆alkyl-, C₅₋₁₂aryl-heteroaryl-heteroC₂₋₆alkenyl-, C₅₋₁₂aryl-heteroC₁₋₆alkyl-heteroaryl-; C₅₋₁₂aryl-heteroC₁₋₆alkyl-C₅₋₁₂aryl-, aryl-heteroC₁₋₆alkyl-heteroaryl-heteroC₂₋₆alkenyl, heteroaryl-heterocylcyl-C₁₋₆alkyl, and C₅₋₁₂aryl-NH—, aryl-NH-heteroaryl-heteroalkenyl-, nitroC₅₋₁₂aryl-, nitroC₅₋₁₂aryl-NH—, nitroC₅₋₁₂aryl-NH-heteroaryl-heteroalkenyl-; and wherein each of said groups can be unsubstituted or substituted with one or more substituents each independently selected from the group comprising halogen, nitro, oxo, C₁₋₆alkyloxy, —C(O)OH, —NH₂, hydroxycarbonylC₁₋₆alkyl, hydroxycarbonylC₂₋₆alkenyl, hydroxyl, C₁₋₆alkyl, C₂₋₆alkenyl, heteroC₁₋₆alkyl, heteroC₂₋₆alkenyl ═S, —SH, C₅₋₁₂aryl, nitroC₅₋₁₂aryl-, nitroC₅₋₁₂aryl-NH, heteroaryl, and heterocyclyl.

Statement 23: The modulator according to any of statements 20 to 22,

wherein cycle D is a heteroaryl, and preferably a heteroaryl selected from the group comprising triazol-2-yl, pyridinyl, 1H-pyrazol-5-yl, pyrrolyl, furanyl, thiophenyl, pyrazolyl, imidazolyl, oxazolyl, isoxazolyl, thiazolyl, isothiazolyl, triazolyl, oxadiazolyl, thiadiazolyl, tetrazolyl, oxatriazolyl, thiatriazolyl, pyrimidyl, pyrazinyl, pyridazinyl, oxazinyl, dioxinyl, thiazinyl, triazinyl, imidazo[2,1-b][1,3]thiazolyl, thieno[3,2-b]furanyl, thieno[3,2-b]thiophenyl, thieno[2,3-d][1,3]thiazolyl, thieno[2,3-d]imidazolyl, tetrazolo[1,5-a]pyridinyl, indolyl, indolizinyl, isoindolyl, benzofuranyl, isobenzofuranyl, benzothiophenyl, isobenzothiophenyl, indazolyl, benzimidazolyl, 1,3-benzoxazolyl, 1,2-benzisoxazolyl, 2,1-benzisoxazolyl, 1,3-benzothiazolyl, 1,2-benzoisothiazolyl, 2,1-benzoisothiazolyl, benzotriazolyl, 1,2,3-benzoxadiazolyl, 2,1,3-benzoxadiazolyl, 1,2,3-benzothiadiazolyl, 2,1,3-benzothiadiazolyl, benzo[d]oxazol-2(3H)-one, 2,3-dihydro-benzofuranyl, thienopyridinyl, purinyl, imidazo[1,2-a]pyridinyl, 6-oxo-pyridazin-1(6H)-yl, 2-oxopyridin-1(2H)-yl, 6-oxo-pyridazin-1(6H)-yl, 2-oxopyridin-1(2H)-yl, 1,3-benzodioxolyl, quinolinyl, isoquinolinyl, cinnolinyl, quinazolinyl, and quinoxalinyl,
or

wherein said cycle D is an aryl, and preferably an aryl selected from the group comprising phenyl, biphenyl, naphthyl, 5,6,7,8-tetrahydronaphthalenyl, 1,2,6,7,8,8a-hexahydroacenaphthylenyl, 1,2-dihydroacenaphthylenyl, and 2,3-dihydro-1H-indenyl.

Statement 24: The modulator according to any of statements 20 to 23, wherein said modulator is any of the compounds 57-74 as selected from Table F. Yes/No in the 5^{th} and 6^{th} columns indicates whether there is, or respectively is not an inhibition of the synthetase (ST) and/or hydrolase (HD) activity.

TABLE F

| Compound | Molport | | | | |
|---|---|---|---|---|---|
| # | ID | ZINC ID | ST | HD | |
| 57 | MolPort- 005-307- 181 | ZINC00001508 1699 | No | Yes | 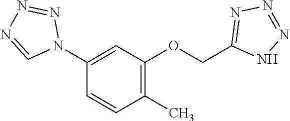 [SMILES: Cc1ccc(-n2cnnn2)cc1OCc1nnn[nH]1] |
| 58 | 005-307- 179 | ZINC00001508 1695 | No | Yes | 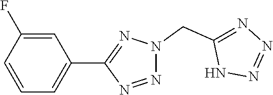 [SMILES: Fc1cccc(-c2nnn(Cc3nnn[nH]3)n2)c1] |
| 59 | MolPort- 038-408- 638 | ZINC00022047 5689 | No | Yes | 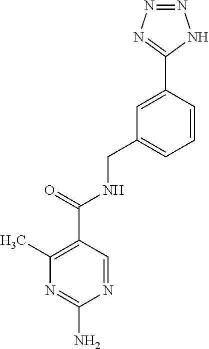 [SMILES: Cc1nc(N)ncc1C(=O)NCc1cccc(-c2nnn[nH]2)c1] |
| 60 | MolPort- 010-337- 764 | ZINC00004736 9304 | No | Yes | 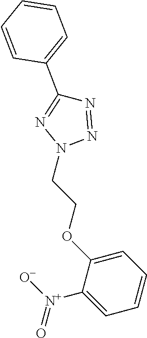 [SMILES: O=[N+]([O-])c1ccccc1OCCn1nnc(-c2ccccc2)n1] |
| 61 | MolPort- 041-865- 652 | ZINC00023751 6182 | No | Yes | 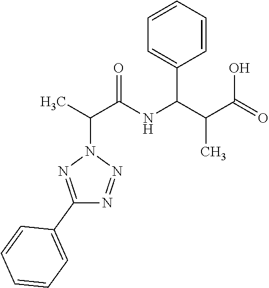 [SMILES: CC(C(=O)O)C(NC(=O)C(C)n1nnc(-c2ccccc2)n1)c1ccccc1] |
| 62 | MolPort- 004-172- 327 | ZINC00000719 2776 | No | Yes | 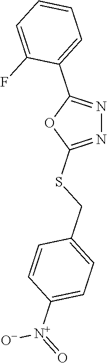 [SMILES: O=[N+]([O-])c1ccc(CSc2nnc(-c3ccccc3F)o2)cc1] |
| 63 | MolPort- 019-682- 935 | ZINC00006547 9810 | No | Yes | 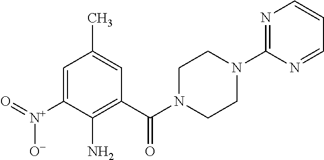 [SMILES: Cc1cc(C(=O)N2CCN(c3ncccn3)CC2)c(N)c([N+](=O)[O-])c1] |
| 64 | MolPort- 006-755- 431 | ZINC00000255 0097 | No | Yes | 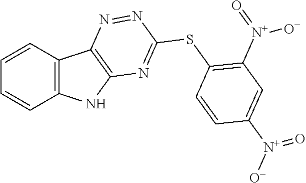 [SMILES: O=[N+]([O-])c1ccc(Sc2nnc3c(n2)[nH]c2ccccc23)c([N+](=O)[O-])c1] |
| 65 | MolPort- 004-275- 317 | ZINC00000264 1443 | No | Yes | 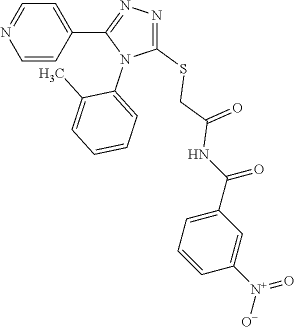 [SMILES: Cc1ccccc1-n1c(SCC(=O)NC(=O)c2cccc([N+](=O)[O-])c2)nnc1-c1ccncc1] |
| 66 | MolPort- 002-690- 320 | ZINC00000887 2830 | Yes | No | 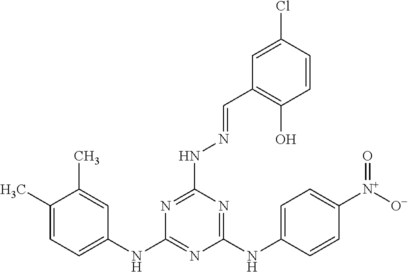 [SMILES: Cc1ccc(Nc2nc(N/N=C/c3cc(Cl)ccc3O)nc(Nc3ccc([N+](=O)[O-])cc3)n2)cc1C] |
| 67 | MolPort- 001-664- 849 | ZINC00000079 7417 | Yes | Yes | 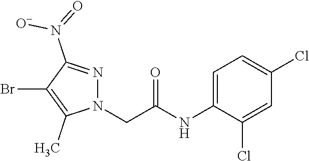 |
| 68 | MolPort- 002-956- 919 | ZINC00001388 0536 | Yes | Yes | 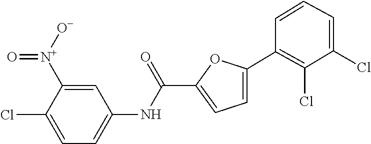 [SMILES: O=C(Nc1ccc(Cl)c([N+](=O)[O-])c1)c1ccc(-c2cccc(Cl)c2Cl)o1] |
| 69 | MolPort- 010-719- 657 | ZINC00006459 0439 | Yes | Yes | 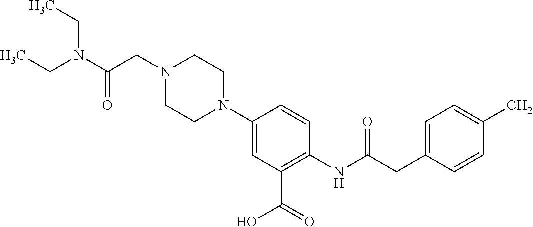 [SMILES: CCN(CC)C(=O)CN1CCN(c2ccc(NC(=O)Cc3ccc(C)cc3)c(C(=O)O)c2)CC1] |
| 70 | MolPort- 010-719- 658 | ZINC00006459 0441 | Yes | Yes | 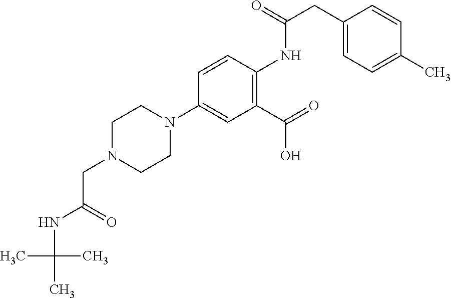 [SMILES: Cc1ccc(CC(=O)Nc2ccc(N3CCN(CC(=O)NC(C)(C)C)CC3)cc2C(=O)O)cc1] |
| 71 | MolPort- 001-656- 638 | ZINC00001396 0070 | Yes | Yes | 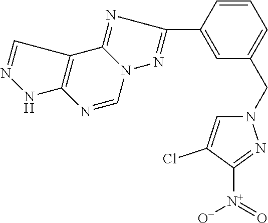 [SMILES: O=[N+]([O-])c1nn(Cc2cccc(-c3nc4c5cn[nH]c5ncn4n3)c2)cc1Cl] |
| 72 | MolPort- 001-497- 383 | ZINC00006819 8266 | Yes | Yes | 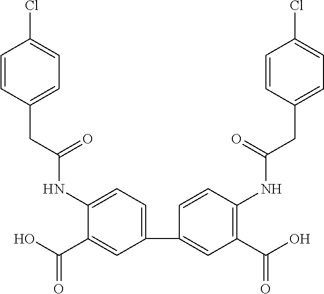 [SMILES: O=C(Cc1ccc(Cl)cc1)Nc1ccc(-c2ccc(NC(=O)Cc3ccc(Cl)cc3)c(C(=O)O)c2)cc1C(=O)O] |
| 73 | MolPort- 010-718- 979 | ZINC00003542 2706 | Yes | Yes | 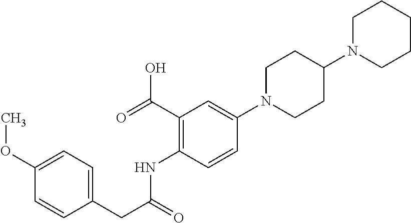 [SMILES: COc1ccc(CC(=O)Nc2ccc(N3CCC(N4CCCCC4)CC3)cc2C(=O)O)cc1] |
| 74 | MolPort- 010-719- 004 | ZINC00003542 2715 | Yes | Yes | 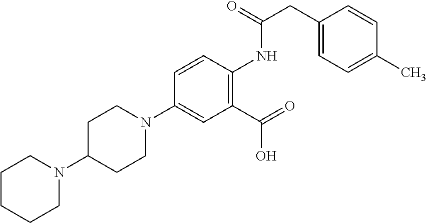 [SMILES: Cc1ccc(CC(=O)Nc2ccc(N3CCC(N4CCCCC4)CC3)cc2C(=O)O)cc1] |

Statement 25: Modulator of Rel hydrolase and/or synthetase activity, wherein said modulator is a compound selected from the group of compounds as given in Table A, Table B, Table C, Table D, Table E, and Table F, or an isomer, preferably a stereo-isomer or a tautomer, a solvate, a salt, preferably a pharmaceutically acceptable salt, or a prodrug thereof.

In Tables A to F the identified compounds were indicated by their respective Molport ID's (https://www.molport.com) or Zinc ID's (https://www.zinc.docking.org).

As indicated above, the “Yes/No” in the 5^{th} and 6^{th} columns of TABLES A to F indicates whether there is, or respectively is not an inhibition of the synthetase (ST) and/or hydrolase (HD) activity. Activities of all the above listed compounds (or other compounds as encompassed by the present invention) were confirmed via in vitro biochemical testing. The action of a candidate compound is evaluated based on its effect on the production of (p)ppGpp. Therefore, in order to assess the potential influence of a candidate compound on synthesis activity, the enzyme was mixed with GDP or GTP and radioactive ATP to produce (p)ppGpp in absence or presence of the candidate compound. Subsequently, the reaction is developed by thin layer chromatography (TLC). In an alternative assay that enables determining the effect of a compound on the hydrolysis activity, radioactive (p)ppGpp is incubated with the enzyme and any decrease of the (p)ppGpp spot on TLC is evaluated when the assay is conducted in presence of the compound and compared to control conditions where no candidate compound is added during incubation. Hence, the inhibitory activity of compounds is assessed based on the effect on the hydrolysis reaction.

The term “alkyl” or “C₁₋₁₈alkyl” as used herein means C₁-C₁₈ normal, secondary, or tertiary, linear, branched or straight hydrocarbon with no site of unsaturation. Examples are methyl, ethyl, 1-propyl (n-propyl), 2-propyl (iPr), 1-butyl, 2-methyl-1-propyl(i-Bu), 2-butyl (s-Bu), 2-dimethyl-2-propyl (t-Bu), 1-pentyl (n-pentyl), 2-pentyl, 3-pentyl, 2-methyl-2-butyl, 3-methyl-2-butyl, 3-methyl-1-butyl, 2-methyl-1-butyl, 1-hexyl, 2-hexyl, 3-hexyl, 2-methyl-2-pentyl, 3-methyl-2-pentyl, 4-methyl-2-pentyl, 3-methyl-3-pentyl, 2-methyl-3-pentyl, 2,3-dimethyl-2-butyl, 3,3-dimethyl-2-butyl, n-heptyl, n-octyl, n-nonyl, n-decyl, n-undecyl, n-dodecyl, n-tridecyl, n-tetradecyl, n-pentadecyl, n-hexadecyl, n-heptadecyl, n-octadecyl, n-nonadecyl, and n-icosyl. In particular embodiments, the term alkyl refers to C₁₋₁₈alkyl (C₁₋₁₈ hydrocarbons), for instance C₁₋₁₂alkyl (C₁₋₁₂ hydrocarbons), yet more in particular to C₁₋₉alkyl (C₁₋₉ hydrocarbons), yet more in particular to C₁₋₆alkyl (C₁₋₆ hydrocarbons) as further defined herein above.

The term “haloalkyl” as a group or part of a group, refers to an alkyl group having the meaning as defined above wherein one, two, or three hydrogen atoms are each replaced with a halogen as defined herein. Non-limiting examples of such haloalkyl groups include chloromethyl, 1-bromoethyl, fluoromethyl, difluoromethyl, trifluoromethyl, 1,1,1-trifluoroethyl and the like.

The term “alkoxy” or “alkyloxy”, as a group or part of a group, refers to a group having the formula —OR^{b} wherein R^{b} is C₁₋₆alkyl as defined herein above. Non-limiting examples of suitable C₁₋₆alkoxy include methoxy, ethoxy, propoxy, isopropoxy, butoxy, isobutoxy, sec-butoxy, tert-butoxy, pentyloxy and hexyloxy. The term “haloalkoxy”, as a group or part of a group, refers to a group of formula —O—R^{c}, wherein R^{c} is haloalkyl as defined herein. Non-limiting examples of suitable haloalkoxy include fluoromethoxy, difluoromethoxy, trifluoromethoxy, 2,2,2-trifluoroethoxy, 1,1,2,2-tetrafluoroethoxy, 2-fluoroethoxy, 2-chloroethoxy, 2,2-difluoroethoxy, 2,2,2-trichloroethoxy, trichloromethoxy, 2-bromoethoxy, pentafluoroethyl, 3,3,3-trichloropropoxy, 4,4,4-trichlorobutoxy.

The term “cycloalkyl” or “C₃₋₁₈cycloalkyl” as used herein and unless otherwise stated means a saturated hydrocarbon monovalent group having from 3 to 18 carbon atoms consisting of or comprising a C₃₋₁₀ monocyclic or C₇₋₁₈ polycyclic saturated hydrocarbon, such as for instance cyclopropyl, cyclobutyl, cyclopentyl, cyclopropylethylene, methylcyclopropylene, cyclohexyl, cycloheptyl, cyclooctyl, cyclooctylmethylene, norbornyl, fenchyl, trimethyltricycloheptyl, decalinyl, adamantyl and the like. In particular embodiments, the term cycloalkyl refers to C₃₋₁₀cycloalkyl (saturated cyclic C₃₋₁₀hydrocarbons), yet more in particular to C₃₋₉cycloalkyl (saturated cyclic C₃₋₉hydrocarbons), still more in particular to C₃₋₆cycloalkyl (saturated cyclic C₃₋₆hydrocarbons) as further defined herein above.

The term “alkenyl” or “C₂₋₁₈alkenyl” as used herein is C₂-C₁₈ normal, secondary or tertiary, linear, branched or straight hydrocarbon with at least one site (usually 1 to 3, preferably 1) of unsaturation, namely a carbon-carbon, sp2 double bond. Examples include, but are not limited to: ethylene or vinyl (—CH═CH₂), allyl (—CH₂CH═CH₂), and 5-hexenyl (—CH₂CH₂CH₂CH₂CH═CH₂). The double bond may be in the cis or trans configuration. In particular embodiments, the term alkenyl refers to C₂₋₁₂alkenyl (C₂₋₁₂hydrocarbons), yet more in particular to C₂₋₉ alkenyl (C₂₋₉ hydrocarbons), still more in particular to C₂₋₆ alkenyl (C₂₋₆hydrocarbons) as further defined herein above with at least one site (usually 1 to 3, preferably 1) of unsaturation, namely a carbon-carbon, sp2 double bond.

The term “alkenyloxy”, as a group or part of a group, refers to a group having the formula —OR^{d} wherein R^{d} is alkenyl as defined herein above.

The term “cycloalkenyl” as used herein refers to a non-aromatic hydrocarbon group having from 5 to 18 carbon atoms with at least one site (usually 1 to 3, preferably 1) of unsaturation, namely a carbon-carbon, sp2 double bond and consisting of or comprising a C₅₋₁₀ monocyclic or C₇₋₁₈ polycyclic hydrocarbon. Examples include, but are not limited to: cyclopentenyl (—C₅H₇), cyclopentenylpropylene, methylcyclohexenylene and cyclohexenyl (—C₆H₉). The double bond may be in the cis or trans configuration. In particular embodiments, the term cycloalkenyl refers to C₅₋₁₂cycloalkenyl (cyclic C₅₋₁₂ hydrocarbons), yet more in particular to C₅₋₉cycloalkenyl (cyclic C₅₋₉ hydrocarbons), still more in particular to C₅₋₆cycloalkenyl (cyclic C₅₋₆hydrocarbons) as further defined herein above with at least one site of unsaturation, namely a carbon-carbon, sp2 double bond.

The term “alkylene” as used herein each refer to a saturated, branched or straight chain hydrocarbon group of 1-18 carbon atoms (more in particular C₁₋₁₂, C₁₋₉ or C₁₋₆ carbon atoms), and having two monovalent group centers derived by the removal of two hydrogen atoms from the same or two different carbon atoms of a parent alkane. Typical alkylene include, but are not limited to: methylene (—CH₂—), 1,2-ethyl (—CH₂CH₂—), 1,3-propyl (—CH₂CH₂CH₂—), 1,4-butyl (—CH₂CH₂CH₂CH₂—), and the like.

The term “alkenylene” as used herein each refer to a branched or straight chain hydrocarbon of 2-18 carbon atoms (more in particular C₂₋₁₂, C₂₋₉ or C₂₋₆ carbon atoms) with at least one site (usually 1 to 3, preferably 1) of unsaturation, namely a carbon-carbon, sp2 double bond, and having two monovalent centers derived by the removal of two hydrogen atoms from the same or two different carbon atoms of a parent alkene.

The term “heteroalkyl” as used herein refers to an alkyl wherein one or more carbon atoms are replaced by one or more atoms independently selected from the group comprising oxygen, nitrogen and sulphur atom with the proviso that said chain may not contain two adjacent O atoms or two adjacent S atoms. Said one or more atoms replacing said carbon atoms may be positioned at the beginning of the hydrocarbon chain, in the hydrocarbon chain or at the end of the hydrocarbon chain. This means that one or more —CH₃ of said alkyl can be replaced by —NH₂ and/or that one or more —CH₂— of said alkyl can be replaced by —NH—, —O— or —S—. In some embodiments the term heteroalkyl encompasses an alkyl which comprises one or more heteroatoms in the hydrocarbon chain, said heteroatoms being selected from the atoms consisting of O, S, and N, whereas the heteroatoms may be positioned at the beginning of the hydrocarbon chain, in the hydrocarbon chain or at the end of the hydrocarbon chain. The S atoms in said chains may be optionally oxidized with one or two oxygen atoms, to afford sulfoxides and sulfones, respectively. Furthermore, the heteroalkyl groups in the compounds of the present invention can contain an oxo or thio group at any carbon or heteroatom that will result in a stable compound. Exemplary heteroalkyl groups include, but are not limited to, alcohols, alkyl ethers, primary, secondary, and tertiary alkyl amines, amides, ketones, esters, alkyl sulfides, and alkyl sulfones. The term heteroalkyl thus comprises but is not limited to —R^{a}—S—; —R^{a}—O—, —R^{a}—N(R^{o})₂—O—R^{b}, —NR^{o}—R^{b}, —R^{a}—O—R^{b}, —O—R^{a}—S—R^{b}, —S—R^{a}, —O—R^{a}—NR⁰R^{b}, —NR^{o}—R^{a}—S—R^{b}, —R^{a}—NR^{o}—R^{b}, —NR⁰R^{a}—S—R^{b}, —S—R^{b}, wherein R^{a} is alkylene, R^{b} is alkyl, and R^{o} is hydrogen or alkyl as defined herein. In particular embodiments, the term encompasses heteroC₁₋₁₂alkyl, heteroC₁₋₉alkyl and heteroC₁₋₆alkyl. In some embodiments heteroalkyl is selected from the group comprising alkyloxy, alkyl-oxy-alkyl, (mono or di)alkylamino, (mono or di-)alkyl-amino-alkyl, alkylthio, and alkyl-thio-alkyl.

The term “heteroalkenyl” as used herein refers to an alkenyl wherein one or more carbon atoms are replaced by one or more atoms independently selected from oxygen, nitrogen and sulphur atom, with the proviso that said chain may not contain two adjacent O atoms or two adjacent S atoms. Said one or more atoms replacing said carbon atoms may be positioned at the beginning of the hydrocarbon chain, in the hydrocarbon chain or at the end of the hydrocarbon chain. This means that one or more —CH₃ of said alkenyl can be replaced by —NH₂, that one or more —CH₂— of said alkenyl can be replaced by —NH—, —O— or —S— and/or that one or more —CH═ of said alkenyl can be replaced by —N═. In some embodiments the term heteroalkenyl encompasses an alkenyl which comprises one or more heteroatoms in the hydrocarbon chain, said heteroatoms being selected from the atoms consisting of O, S, and N, whereas the heteroatoms may be positioned at the beginning of the hydrocarbon chain, in the hydrocarbon chain or at the end of the hydrocarbon chain. The S atoms in said chains may be optionally oxidized with one or two oxygen atoms, to afford sulfoxides and sulfones, respectively. Furthermore, the heteroalkyl groups in the compounds of the present invention can contain an oxo or thio group at any carbon or heteroatom that will result in a stable compound. The term heteroalkenyl thus comprises imines, —O-alkenyl, —NH-alkenyl, —N(alkenyl)₂, —N(alkyl)(alkenyl), and —S-alkenyl. The term heteroalkenyl thus comprises but is not limited to —R^{d}—O—, —O—R^{d}, —NH—(R^{d}), —N═R^{d}, —N(R^{d}))₂, —N(R^{b})(R^{d}), —NH—NH—R^{d}, —R^{d}═N—N═R^{d}, —R^{d}═N—N═, —R^{d}—S—, —S—R^{d} wherein R^{b} is alkyl and R^{d} is alkenyl as defined herein. In particular embodiments, the term heteroalkenyl encompasses heteroC₂₋₁₈alkenyl, heteroC₂₋₁₂alkenyl, heteroC₂₋₉alkenyl and heteroC₂₋₆alkenyl. In some embodiments heteroalkenyl is selected from the group comprising alkenyloxy, alkenyl-oxy-alkenyl, (mono or di-)alkenylamino, (mono or di-)alkenyl-amino-alkenyl, alkenylthio, and alkenyl-thio-alkenyl.

The term “heteroalkylene” as used herein refers to an alkylene wherein one or more carbon atoms are replaced by one or more oxygen, nitrogen or sulphur atoms, with the proviso that said chain may not contain two adjacent O atoms or two adjacent S atoms. This means that one or more —CH₃ of said alkylene can be replaced by —NH₂ and/or that one or more —CH₂— of said alkylene can be replaced by —NH—, —O— or —S—. The S atoms in said chains may be optionally oxidized with one or two oxygen atoms, to afford sulfoxides and sulfones, respectively. Furthermore, the heteroalkylene groups in the compounds of the present invention can contain an oxo or thio group at any carbon or heteroatom that will result in a stable compound.

The term “heteroalkenylene” as used herein refers to an alkenylene wherein one or more carbon atoms are replaced by one or more oxygen, nitrogen or sulphur atoms, with the proviso that said chain may not contain two adjacent O atoms or two adjacent S atoms. This means that one or more —CH₃ of said alkenylene can be replaced by —NH₂, that one or more —CH₂— of said alkenylene can be replaced by —NH—, —O— or —S— and/or that one or more —CH═ of said alkenylene can be replaced by —N═. The S atoms in said chains may be optionally oxidized with one or two oxygen atoms, to afford sulfoxides and sulfones, respectively. Furthermore, the heteroalkenylene groups in the compounds of the present invention can contain an oxo or thio group at any carbon or heteroatom that will result in a stable compound.

The term “aryl” as used herein means an aromatic hydrocarbon of 5-20 carbon atoms derived by the removal of hydrogen from a carbon atom of an aromatic ring system. Examples of aryl groups include, but are not limited to 1 ring, or 2 or 3 rings fused together, of which at least one ring is aromatic. Such ring can be derived from benzene, naphthalene, anthracene, biphenyl, 2,3-dihydro-1H-indenyl, 5,6,7,8-tetrahydronaphthalenyl, 1,2,6,7,8,8a-hexahydroacenaphthylenyl, 1,2-dihydroacenaphthylenyl, and the like. Particular aryl groups are phenyl and naphthyl, especially phenyl.

The term “aryloxy”, as a group or part of a group, refers to a group having the formula —OR^{g} wherein R^{g} is aryl as defined herein above.

The term “arylalkyl” or “arylalkyl-” as used herein refers to an alkyl in which one of the hydrogen atoms bonded to a carbon atom, typically a terminal or sp3 carbon atom, is replaced with an aryl. Typical arylalkyl groups include, but are not limited to, benzyl, 2-phenylethan-1-yl, 2-phenylethen-1-yl, naphthylmethyl, 2-naphthylethyl, and the like. The arylalkyl group can comprise 6 to 20 carbon atoms, e.g. the alkyl moiety of the arylalkyl group is 1 to 6 carbon atoms and the aryl moiety is 5 to 14 carbon atoms.

The term “arylalkyloxy”, as a group or part of a group, refers to a group having the formula —O—R^{a}—R^{g} wherein R^{g} is aryl, and R^{a} is alkylene as defined herein above.

The term “arylalkenyl” or “arylalkenyl-” as used herein refers to an alkenyl in which one of the hydrogen atoms bonded to a carbon atom, is replaced with an aryl.

The term “aryl-alkenyl” as a group or part of a group refers to an alkenyl in which one of the hydrogen atoms bonded to a carbon atom, is replaced with an aryl. The aryl-alkenyl group can comprise 6 to 20 atoms, e.g. the alkenyl moiety of the aryl-alkenyl group can comprise 1 to 6 carbon atoms and the aryl moiety can comprise 5 to 14 atoms, such as ═CH—R^{g}, wherein R^{g} is aryl as defined herein above.

The term “arylheteroalkyl” or “arylheteroalkyl-” as used herein refers to a heteroalkyl in which one of the hydrogen atoms bonded to a carbon atom, typically a terminal or sp3 carbon atom, is replaced with an aryl. The arylheteroalkyl group can comprise 6 to 20 carbon atoms, e.g. the heteroalkyl moiety of the arylheteroalkyl group is 1 to 6 carbon atoms and the aryl moiety is 5 to 14 carbon atoms. In some embodiments arylheteroalkyl is selected from the group comprising aryl-O-alkyl, arylalkyl-O-alkyl, aryl-NH-alkyl, aryl-N(alkyl)₂, arylalkyl-NH-alkyl, arylalkyl-N-(alkyl)₂, aryl-S-alkyl, and arylalkyl-S-alkyl.

The term “arylheteroalkenyl” or “arylheteroalkenyl-” as used herein refers to a heteroalkenyl in which one of the hydrogen atoms bonded to a carbon atom, is replaced with an aryl. The arylheteroalkenyl group can comprise 6 to 20 carbon atoms, e.g. the heteroalkenyl moiety of the arylheteroalkenyl group is 1 to 6 carbon atoms and the aryl moiety is 5 to 14 carbon atoms. In some embodiments arylheteroalkenyl is selected from the group comprising aryl-O-alkenyl, arylalkenyl-O-alkenyl, aryl-NH-alkenyl, arylalkenyl-NH-alkenyl, aryl-S-alkenyl, and arylalkenyl-S-alkenyl.

The term “heterocyclyl” as used herein refer to non-aromatic, fully saturated or partially unsaturated ring system of 3 to 18 atoms including at least one N, O, S, or P (for example, 3 to 7 member monocyclic, 7 to 11 member bicyclic, or comprising a total of 3 to 10 ring atoms) wherein at least one ring is a heterocyclyl and wherein said ring may be fused to an aryl, cycloalkyl, heteroaryl and/or heterocyclyl ring. Each ring of the heterocyclyl may have 1, 2, 3 or 4 heteroatoms selected from N, O and/or S, where the N and S heteroatoms may optionally be oxidized and the N heteroatoms may optionally be quaternized; and wherein at least one carbon atom of heterocyclyl can be oxidized to form at least one C═O. The heterocyclic may be attached at any heteroatom or carbon atom of the ring or ring system, where valence allows. The rings of multi-ring heterocyclyls may be fused, bridged and/or joined through one or more spiro atoms.

Non limiting exemplary heterocyclic groups include piperidinyl, piperazinyl, homopiperazinyl, morpholinyl, tetrahydropyranyl, tetrahydrofuranyl, pyrrolidinyl, aziridinyl, oxiranyl, thiiranyl, azetidinyl, oxetanyl, thietanyl, 2-imidazolinyl, pyrazolidinyl imidazolidinyl, isoxazolinyl, oxazolidinyl, isoxazolidinyl, thiazolidinyl, isothiazolidinyl, succinimidyl, 3H-indolyl, indolinyl, isoindolinyl, chromanyl (also known as 3,4-dihydrobenzo[b]pyranyl), 2H-pyrrolyl, 1-pyrrolinyl, 2-pyrrolinyl, 3-pyrrolinyl, 4H-quinolizinyl, 2-oxopiperazinyl, 2-pyrazolinyl, 3-pyrazolinyl, tetrahydro-2H-pyranyl, 2H-pyranyl, 4H-pyranyl, 3,4-dihydro-2H-pyranyl, 3-dioxolanyl, 1,4-dioxanyl, 2,5-dioximidazolidinyl, 2-oxopiperidinyl, 2-oxopyrrolodinyl, indolinyl, tetrahydrothiophenyl, tetrahydroquinolinyl, tetrahydroisoquinolin-1-yl, tetrahydroisoquinolin-2-yl, tetrahydroisoquinolin-3-yl, tetrahydroisoquinolin-4-yl, thiomorpholin-4-yl, thiomorpholin-4-ylsulfoxide, thiomorpholin-4-ylsulfone, 1,3-dioxolanyl, 1,4-oxathianyl, 1,4-dithianyl, 1,3,5-trioxanyl, 1H-pyrrolizinyl, tetrahydro-1,1-dioxothiophenyl, N-formylpiperazinyl, and morpholin-4-yl. The term “aziridinyl” as used herein includes aziridin-1-yl and aziridin-2-yl. The term “oxyranyl” as used herein includes oxyranyl-2-yl. The term “thiiranyl” as used herein includes thiiran-2-yl. The term “azetidinyl” as used herein includes azetidin-1-yl, azetidin-2-yl and azetidin-3-yl. The term “oxetanyl” as used herein includes oxetan-2-yl and oxetan-3-yl. The term “thietanyl” as used herein includes thietan-2-yl and thietan-3-yl. The term “pyrrolidinyl” as used herein includes pyrrolidin-1-yl, pyrrolidin-2-yl and pyrrolidin-3-yl. The term “tetrahydrofuranyl” as used herein includes tetrahydrofuran-2-yl and tetrahydrofuran-3-yl. The term “tetrahydrothiophenyl” as used herein includes tetrahydrothiophen-2-yl and tetrahydrothiophen-3-yl. The term “succinimidyl” as used herein includes succinimid-1-yl and succininmid-3-yl. The term “dihydropyrrolyl” as used herein includes 2,3-dihydropyrrol-1-yl, 2,3-dihydro-1H-pyrrol-2-yl, 2,3-dihydro-1H-pyrrol-3-yl, 2,5-dihydropyrrol-1-yl, 2,5-dihydro-1H-pyrrol-3-yl and 2,5-dihydropyrrol-5-yl. The term “2H-pyrrolyl” as used herein includes 2H-pyrrol-2-yl, 2H-pyrrol-3-yl, 2H-pyrrol-4-yl and 2H-pyrrol-5-yl. The term “3H-pyrrolyl” as used herein includes 3H-pyrrol-2-yl, 3H-pyrrol-3-yl, 3H-pyrrol-4-yl and 3H-pyrrol-5-yl. The term “dihydrofuranyl” as used herein includes 2,3-dihydrofuran-2-yl, 2,3-dihydrofuran-3-yl, 2,3-dihydrofuran-4-yl, 2,3-dihydrofuran-5-yl, 2,5-dihydrofuran-2-yl, 2,5-dihydrofuran-3-yl, 2,5-dihydrofuran-4-yl and 2,5-dihydrofuran-5-yl. The term “dihydrothiophenyl” as used herein includes 2,3-dihydrothiophen-2-yl, 2,3-dihydrothiophen-3-yl, 2,3-dihydrothiophen-4-yl, 2,3-dihydrothiophen-5-yl, 2,5-dihydrothiophen-2-yl, 2,5-dihydrothiophen-3-yl, 2,5-dihydrothiophen-4-yl and 2,5-dihydrothiophen-5-yl. The term “imidazolidinyl” as used herein includes imidazolidin-1-yl, imidazolidin-2-yl and imidazolidin-4-yl. The term “pyrazolidinyl” as used herein includes pyrazolidin-1-yl, pyrazolidin-3-yl and pyrazolidin-4-yl. The term “imidazolinyl” as used herein includes imidazolin-1-yl, imidazolin-2-yl, imidazolin-4-yl and imidazolin-5-yl. The term “pyrazolinyl” as used herein includes 1-pyrazolin-3-yl, 1-pyrazolin-4-yl, 2-pyrazolin-1-yl, 2-pyrazolin-3-yl, 2-pyrazolin-4-yl, 2-pyrazolin-5-yl, 3-pyrazolin-1-yl, 3-pyrazolin-2-yl, 3-pyrazolin-3-yl, 3-pyrazolin-4-yl and 3-pyrazolin-5-yl. The term “dioxolanyl” also known as “1,3-dioxolanyl” as used herein includes dioxolan-2-yl, dioxolan-4-yl and dioxolan-5-yl. The term “dioxolyl” also known as “1,3-dioxolyl” as used herein includes dioxol-2-yl, dioxol-4-yl and dioxol-5-yl. The term “oxazolidinyl” as used herein includes oxazolidin-2-yl, oxazolidin-3-yl, oxazolidin-4-yl and oxazolidin-5-yl. The term “isoxazolidinyl” as used herein includes isoxazolidin-2-yl, isoxazolidin-3-yl, isoxazolidin-4-yl and isoxazolidin-5-yl. The term “oxazolinyl” as used herein includes 2-oxazolinyl-2-yl, 2-oxazolinyl-4-yl, 2-oxazolinyl-5-yl, 3-oxazolinyl-2-yl, 3-oxazolinyl-4-yl, 3-oxazolinyl-5-yl, 4-oxazolinyl-2-yl, 4-oxazolinyl-3-yl, 4-oxazolinyl-4-yl and 4-oxazolinyl-5-yl. The term “isoxazolinyl” as used herein includes 2-isoxazolinyl-3-yl, 2-isoxazolinyl-4-yl, 2-isoxazolinyl-5-yl, 3-isoxazolinyl-3-yl, 3-isoxazolinyl-4-yl, 3-isoxazolinyl-5-yl, 4-isoxazolinyl-2-yl, 4-isoxazolinyl-3-yl, 4-isoxazolinyl-4-yl and 4-isoxazolinyl-5-yl. The term “thiazolidinyl” as used herein includes thiazolidin-2-yl, thiazolidin-3-yl, thiazolidin-4-yl and thiazolidin-5-yl. The term “isothiazolidinyl” as used herein includes isothiazolidin-2-yl, isothiazolidin-3-yl, isothiazolidin-4-yl and isothiazolidin-5-yl. The term “thiazolinyl” as used herein includes 2-thiazolinyl-2-yl, 2-thiazolinyl-4-yl, 2-thiazolinyl-5-yl, 3-thiazolinyl-2-yl, 3-thiazolinyl-4-yl, 3-thiazolinyl-5-yl, 4-thiazolinyl-2-yl, 4-thiazolinyl-3-yl, 4-thiazolinyl-4-yl and 4-thiazolinyl-5-yl. The term “isothiazolinyl” as used herein includes 2-isothiazolinyl-3-yl, 2-isothiazolinyl-4-yl, 2-isothiazolinyl-5-yl, 3-isothiazolinyl-3-yl, 3-isothiazolinyl-4-yl, 3-isothiazolinyl-5-yl, 4-isothiazolinyl-2-yl, 4-isothiazolinyl-3-yl, 4-isothiazolinyl-4-yl and 4-isothiazolinyl-5-yl. The term “piperidyl” also known as “piperidinyl” as used herein includes piperid-1-yl, piperid-2-yl, piperid-3-yl and piperid-4-yl. The term “dihydropyridinyl” as used herein includes 1,2-dihydropyridin-1-yl, 1,2-dihydropyridin-2-yl, 1,2-dihydropyridin-3-yl, 1,2-dihydropyridin-4-yl, 1,2-dihydropyridin-5-yl, 1,2-dihydropyridin-6-yl, 1,4-dihydropyridin-1-yl, 1,4-dihydropyridin-2-yl, 1,4-dihydropyridin-3-yl, 1,4-dihydropyridin-4-yl, 2,3-dihydropyridin-2-yl, 2,3-dihydropyridin-3-yl, 2,3-dihydropyridin-4-yl, 2,3-dihydropyridin-5-yl, 2,3-dihydropyridin-6-yl, 2,5-dihydropyridin-2-yl, 2,5-dihydropyridin-3-yl, 2,5-dihydropyridin-4-yl, 2,5-dihydropyridin-5-yl, 2,5-dihydropyridin-6-yl, 3,4-dihydropyridin-2-yl, 3,4-dihydropyridin-3-yl, 3,4-dihydropyridin-4-yl, 3,4-dihydropyridin-5-yl and 3,4-dihydropyridin-6-yl. The term “tetrahydropyridinyl” as used herein includes 1,2,3,4-tetrahydropyridin-1-yl, 1,2,3,4-tetrahydropyridin-2-yl, 1,2,3,4-tetrahydropyridin-3-yl, 1,2,3,4-tetrahydropyridin-4-yl, 1,2,3,4-tetrahydropyridin-5-yl, 1,2,3,4-tetrahydropyridin-6-yl, 1,2,3,6-tetrahydropyridin-1-yl, 1,2,3,6-tetrahydropyridin-2-yl, 1,2,3,6-tetrahydropyridin-3-yl, 1,2,3,6-tetrahydropyridin-4-yl, 1,2,3,6-tetrahydropyridin-5-yl, 1,2,3,6-tetrahydropyridin-6-yl, 2,3,4,5-tetrahydropyridin-2-yl, 2,3,4,5-tetrahydropyridin-3-yl, 2,3,4,5-tetrahydropyridin-3-yl, 2,3,4,5-tetrahydropyridin-4-yl, 2,3,4,5-tetrahydropyridin-5-yl and 2,3,4,5-tetrahydropyridin-6-yl. The term “tetrahydropyranyl” also known as “oxanyl” or “tetrahydro-2H-pyranyl”, as used herein includes tetrahydropyran-2-yl, tetrahydropyran-3-yl and tetrahydropyran-4-yl. The term “2H-pyranyl” as used herein includes 2H-pyran-2-yl, 2H-pyran-3-yl, 2H-pyran-4-yl, 2H-pyran-5-yl and 2H-pyran-6-yl. The term “4H-pyranyl” as used herein includes 4H-pyran-2-yl, 4H-pyran-3-yl and 4H-pyran-4-yl. The term “3,4-dihydro-2H-pyranyl” as used herein includes 3,4-dihydro-2H-pyran-2-yl, 3,4-dihydro-2H-pyran-3-yl, 3,4-dihydro-2H-pyran-4-yl, 3,4-dihydro-2H-pyran-5-yl and 3,4-dihydro-2H-pyran-6-yl. The term “3,6-dihydro-2H-pyranyl” as used herein includes 3,6-dihydro-2H-pyran-2-yl, 3,6-dihydro-2H-pyran-3-yl, 3,6-dihydro-2H-pyran-4-yl, 3,6-dihydro-2H-pyran-5-yl and 3,6-dihydro-2H-pyran-6-yl. The term “tetrahydrothiophenyl”, as used herein includes tetrahydrothiophen-2-yl, tetrahydrothiophenyl-3-yl and tetrahydrothiophenyl-4-yl. The term “2H-thiopyranyl” as used herein includes 2H-thiopyran-2-yl, 2H-thiopyran-3-yl, 2H-thiopyran-4-yl, 2H-thiopyran-5-yl and 2H-thiopyran-6-yl. The term “4H-thiopyranyl” as used herein includes 4H-thiopyran-2-yl, 4H-thiopyran-3-yl and 4H-thiopyran-4-yl. The term “3,4-dihydro-2H-thiopyranyl” as used herein includes 3,4-dihydro-2H-thiopyran-2-yl, 3,4-dihydro-2H-thiopyran-3-yl, 3,4-dihydro-2H-thiopyran-4-yl, 3,4-dihydro-2H-thiopyran-5-yl and 3,4-dihydro-2H-thiopyran-6-yl. The term “3,6-dihydro-2H-thiopyranyl” as used herein includes 3,6-dihydro-2H-thiopyran-2-yl, 3,6-dihydro-2H-thiopyran-3-yl, 3,6-dihydro-2H-thiopyran-4-yl, 3,6-dihydro-2H-thiopyran-5-yl and 3,6-dihydro-2H-thiopyran-6-yl. The term “piperazinyl” also known as “piperazidinyl” as used herein includes piperazin-1-yl and piperazin-2-yl. The term “morpholinyl” as used herein includes morpholin-2-yl, morpholin-3-yl and morpholin-4-yl. The term “thiomorpholinyl” as used herein includes thiomorpholin-2-yl, thiomorpholin-3-yl and thiomorpholin-4-yl. The term “dioxanyl” as used herein includes 1,2-dioxan-3-yl, 1,2-dioxan-4-yl, 1,3-dioxan-2-yl, 1,3-dioxan-4-yl, 1,3-dioxan-5-yl and 1,4-dioxan-2-yl. The term “dithianyl” as used herein includes 1,2-dithian-3-yl, 1,2-dithian-4-yl, 1,3-dithian-2-yl, 1,3-dithian-4-yl, 1,3-dithian-5-yl and 1,4-dithian-2-yl. The term “oxathianyl” as used herein includes oxathian-2-yl and oxathian-3-yl. The term “trioxanyl” as used herein includes 1,2,3-trioxan-4-yl, 1,2,3-trioxay-5-yl, 1,2,4-trioxay-3-yl, 1,2,4-trioxay-5-yl, 1,2,4-trioxay-6-yl and 1,3,4-trioxay-2-yl. The term “azepanyl” as used herein includes azepan-1-yl, azepan-2-yl, azepan-1-yl, azepan-3-yl and azepan-4-yl. The term “homopiperazinyl” as used herein includes homopiperazin-1-yl, homopiperazin-2-yl, homopiperazin-3-yl and homopiperazin-4-yl. The term “indolinyl” as used herein includes indolin-1-yl, indolin-2-yl, indolin-3-yl, indolin-4-yl, indolin-5-yl, indolin-6-yl, and indolin-7-yl. The term “quinolizinyl” as used herein includes quinolizidin yl, quinolizidin-2-yl, quinolizidin-3-yl and quinolizidin-4-yl. The term “isoindolinyl” as used herein includes isoindolin-1-yl, isoindolin-2-yl, isoindolin-3-yl, isoindolin-4-yl, isoindolin-5-yl, isoindolin-6-yl, and isoindolin-7-yl. The term “3H-indolyl” as used herein includes 3H-indol-2-yl, 3H-indol-3-yl, 3H-indol-4-yl, 3H-indol-5-yl, 3H-indol-6-yl, and 3H-indol-7-yl. The term “quinolizinyl” as used herein includes quinolizidin-1-yl, quinolizidin-2-yl, quinolizidin-3-yl and quinolizidin-4-yl. The term “quinolizinyl” as used herein includes quinolizidin-1-yl, quinolizidin-2-yl, quinolizidin-3-yl and quinolizidin-4-yl. The term “tetrahydroquinolinyl” as used herein includes tetrahydroquinolin-1-yl, tetrahydroquinolin-2-yl, tetrahydroquinolin-3-yl, tetrahydroquinolin-4-yl, tetrahydro quinolin-5-yl, tetrahydroquinolin-6-yl, tetrahydroquinolin-7-yl and tetrahydroquinolin-8-yl. The term “tetrahydroisoquinolinyl” as used herein includes tetrahydroisoquinolin-1-yl, tetrahydroisoquinolin-2-yl, tetrahydroisoquinolin-3-yl, tetrahydroisoquinolin-4-yl, tetrahydroisoquinolin-5-yl, tetrahydroisoquinolin-6-yl, tetrahydroisoquinolin-7-yl and tetrahydroisoquinolin-8-yl. The term “chromanyl” as used herein includes chroman-2-yl, chroman-3-yl, chroman-4-yl, chroman-5-yl, chroman-6-yl, chroman-7-yl and chroman-8-yl. The term “1H-pyrrolizine” as used herein includes 1H-pyrrolizin-1-yl, 1H-pyrrolizin-2-yl, 1H-pyrrolizin-3-yl, 1H-pyrrolizin-5-yl, 1H-pyrrolizin-6-yl and 1H-pyrrolizin-7-yl. The term “3H-pyrrolizine” as used herein includes 3H-pyrrolizin-1-yl, 3H-pyrrolizin-2-yl, 3H-pyrrolizin-3-yl, 3H-pyrrolizin-5-yl, 3H-pyrrolizin-6-yl and 3H-pyrrolizin-7-yl.

The term “heteroaryl” refers but is not limited to an aromatic ring system of 5 to 18 atoms including at least one N, O, S, or P, containing 1 or more rings, such as 1 or 2 or 3 or 4 rings, which can be fused together or linked covalently, each ring typically containing 5 to 6 atoms; at least one of said ring is aromatic, where the N and S heteroatoms may optionally be oxidized and the N heteroatoms may optionally be quaternized, and wherein at least one carbon atom of said heteroaryl can be oxidized to form at least one C═O. Such rings may be fused to an aryl, cycloalkyl, heteroaryl and/or heterocyclyl ring.

Non-limiting examples of such heteroaryl, include: triazol-2-yl, pyridinyl, 1H-pyrazol-5-yl, pyrrolyl, furanyl, thiophenyl, pyrazolyl, imidazolyl, oxazolyl, isoxazolyl, thiazolyl, isothiazolyl, triazolyl, oxadiazolyl, thiadiazolyl, tetrazolyl, oxatriazolyl, thiatriazolyl, pyrimidyl, pyrazinyl, pyridazinyl, oxazinyl, dioxinyl, thiazinyl, triazinyl, imidazo[2,1-b][1,3]thiazolyl, thieno[3,2-b]furanyl, thieno[3,2-b]thiophenyl, thieno[2,3-d][1,3]thiazolyl, thieno[2,3-d]imidazolyl, tetrazolo[1,5-a]pyridinyl, indolyl, indolizinyl, isoindolyl, benzofuranyl, isobenzofuranyl, benzothiophenyl, isobenzothiophenyl, indazolyl, benzimidazolyl, 1,3-benzoxazolyl, 1,2-benzisoxazolyl, 2,1-benzisoxazolyl, 1,3-benzothiazolyl, 1,2-benzoisothiazolyl, 2,1-benzoisothiazolyl, benzotriazolyl, 1,2,3-benzoxadiazolyl, 2,1,3-benzoxadiazolyl, 1,2,3-benzothiadiazolyl, 2,1,3-benzothiadiazolyl, benzo[d]oxazol-2(3H)-one, 2,3-dihydro-benzofuranyl, thienopyridinyl, purinyl, imidazo[1,2-a]pyridinyl, 6-oxo-pyridazin-1(6H)-yl, 2-oxopyridin-1(2H)-yl, 6-oxo-pyridazin-1(6H)-yl, 2-oxopyridin-1(2H)-yl, 1,3-benzodioxolyl, quinolinyl, isoquinolinyl, cinnolinyl, quinazolinyl, quinoxalinyl; preferably said heteroaryl group is selected from the group comprising pyridyl, 1,3-benzodioxolyl, benzo[d]oxazol-2(3H)-one, 2,3-dihydro-benzofuranyl, pyrazinyl, pyrazolyl, pyrrolyl, isoxazolyl, thiophenyl, imidazolyl, benzimidazolyl, pyrimidinyl, s-triazinyl, oxazolyl, isothiazolyl, furyl, thienyl, triazolyl thiazolyl, 5H-[1,2,4]triazino[5,6-b]indole, and 3,5,6,8,10,11-hexaazatricyclo[7.3.0.0²,⁶]dodeca-1(9), 2,4,7,11-pentaenyl.

The term “pyrrolyl” (also called azolyl) as used herein includes pyrrol-1-yl, pyrrol-2-yl and pyrrol-3-yl. The term “furanyl” (also called “furyl”) as used herein includes furan-2-yl and furan-3-yl (also called furan-2-yl and furan-3-yl). The term “thiophenyl” (also called “thienyl”) as used herein includes thiophen-2-yl and thiophen-3-yl (also called thien-2-yl and thien-3-yl). The term “pyrazolyl” (also called 1H-pyrazolyl and 1,2-diazolyl) as used herein includes pyrazol-1-yl, pyrazol-3-yl or 1H-pyrazol-5-yl, pyrazol-4-yl and pyrazol-5-yl. The term “imidazolyl” as used herein includes imidazol-1-yl, imidazol-2-yl, imidazol-4-yl and imidazol-5-yl. The term “oxazolyl” (also called 1,3-oxazolyl) as used herein includes oxazol-2-yl, oxazol-4-yl and oxazol-5-yl. The term “isoxazolyl” (also called 1,2-oxazolyl), as used herein includes isoxazol-3-yl, isoxazol-4-yl, and isoxazol-5-yl. The term “thiazolyl” (also called 1,3-thiazolyl),as used herein includes thiazol-2-yl, thiazol-4-yl and thiazol-5-yl (also called 2-thiazolyl, 4-thiazolyl and 5-thiazolyl). The term “isothiazolyl” (also called 1,2-thiazolyl) as used herein includes isothiazol-3-yl, isothiazol-4-yl, and isothiazol-5-yl. The term “triazolyl” as used herein includes triazol-2-yl, 1H-triazolyl and 4H-1,2,4-triazolyl, “1H-triazolyl” includes 1H-1,2,3-triazol-1-yl, 1H-1,2,3-triazol-4-yl, 1H-1,2,3-triazol-5-yl, 1H-1,2,4-triazol-1-yl, 1H-1,2,4-triazol-3-yl and 1H-1,2,4-triazol-5-yl. “4H-1,2,4-triazolyl” includes 4H-1,2,4-triazol-4-yl, and 4H-1,2,4-triazol-3-yl. The term “oxadiazolyl” as used herein includes 1,2,3-oxadiazol-4-yl, 1,2,3-oxadiazol-5-yl, 1,2,4-oxadiazol-3-yl, 1,2,4-oxadiazol-5-yl, 1,2,5-oxadiazol-3-yl and 1,3,4-oxadiazol-2-yl. The term “thiadiazolyl” as used herein includes 1,2,3-thiadiazol-4-yl, 1,2,3-thiadiazol-5-yl, 1,2,4-thiadiazol-3-yl, 1,2,4-thiadiazol-5-yl, 1,2,5-thiadiazol-3-yl (also called furazan-3-yl) and 1,3,4-thiadiazol-2-yl. The term “tetrazolyl” as used herein includes 1H-tetrazol-1-yl, 1H-tetrazol-5-yl, 2H-tetrazol-2-yl, and 2H-tetrazol-5-yl. The term “oxatriazolyl” as used herein includes 1,2,3,4-oxatriazol-5-yl and 1,2,3,5-oxatriazol-4-yl. The term “thiatriazolyl” as used herein includes 1,2,3,4-thiatriazol-5-yl and 1,2,3,5-thiatriazol-4-yl. The term “pyridinyl” (also called “pyridyl”) as used herein includes pyridin-2-yl, pyridin-3-yl and pyridin-4-yl (also called 2-pyridyl, 3-pyridyl and 4-pyridyl). The term “pyrimidyl” as used herein includes pyrimid-2-yl, pyrimid-4-yl, pyrimid-5-yl and pyrimid-6-yl. The term “pyrazinyl” as used herein includes pyrazin yl and pyrazin-3-yl. The term “pyridazinyl as used herein includes pyridazin-3-yl and pyridazin-4-yl. The term “oxazinyl” (also called “1,4-oxazinyl”) as used herein includes 1,4-oxazin-4-yl and 1,4-oxazin-5-yl. The term “dioxinyl” (also called “1,4-dioxinyl”) as used herein includes 1,4-dioxin-2-yl and 1,4-dioxin-3-yl. The term “thiazinyl” (also called “1,4-thiazinyl”) as used herein includes 1,4-thiazin-2-yl, 1,4-thiazin-3-yl, 1,4-thiazin-4-yl, 1,4-thiazin-5-yl and 1,4-thiazin-6-yl. The term “triazinyl” as used herein includes 1,3,5-triazin-2-yl, 1,2,4-triazin-3-yl, 1,2,4-triazin-5-yl, 1,2,4-triazin-6-yl, 1,2,3-triazin-4-yl and 1,2,3-triazin-5-yl. The term “imidazo[2,1-b][1,3]thiazolyl” as used herein includes imidazo[2,1-b][1,3]thiazol-2-yl, imidazo[2,1-b][1,3]thiazol-3-yl, imidazo[2,1-b][1,3]thiazol-5-yl and imidazo[2,1-b][1,3]thiazol-6-yl. The term “thieno[3,2-b]furanyl” as used herein includes thieno[3,2-b]furan-2-yl, thieno[3,2-b]furan-3-yl, thieno[3,2-b]furan-4-yl, and thieno[3,2-b]furan-5-yl. The term “thieno[3,2-b]thiophenyl” as used herein includes thieno[3,2-b]thien-2-yl, thieno[3,2-b]thien-3-yl, thieno[3,2-b]thien-5-yl and thieno[3,2-b]thien-6-yl. The term “thieno[2,3-d][1,3]thiazolyl” as used herein includes thieno[2,3-d][1,3]thiazol-2-yl, thieno[2,3-d][1,3]thiazol-5-yl and thieno[2,3-d][1,3]thiazol-6-yl. The term “thieno[2,3-d]imidazolyl” as used herein includes thieno[2,3-d]imidazol-2-yl, thieno[2,3-d]imidazol-4-yl and thieno[2,3-d]imidazol-5-yl. The term “tetrazolo[1,5-a]pyridinyl” as used herein includes tetrazolo[1,5-a]pyridine-5-yl, tetrazolo[1,5-a]pyridine-6-yl, tetrazolo[1,5-a]pyridine-7-yl, and tetrazolo[1,5-a]pyridine-8-yl. The term “indolyl” as used herein includes indol-1-yl, indol-2-yl, indol-3-yl,-indol-4-yl, indol-5-yl, indol-6-yl and indol-7-yl. The term “indolizinyl” as used herein includes indolizin-1-yl, indolizin-2-yl, indolizin-3-yl, indolizin-5-yl, indolizin-6-yl, indolizin-7-yl, and indolizin-8-yl. The term “isoindolyl” as used herein includes isoindol-1-yl, isoindol-2-yl, isoindol-3-yl, isoindol-4-yl, isoindol-5-yl, isoindol-6-yl and isoindol-7-yl. The term “benzofuranyl” (also called benzo[b]furanyl) as used herein includes benzofuran-2-yl, benzofuran-3-yl, benzofuran-4-yl, benzofuran-5-yl, benzofuran-6-yl and benzofuran-7-yl. The term “isobenzofuranyl” (also called benzo[c]furanyl) as used herein includes isobenzofuran-1-yl, isobenzofuran-3-yl, isobenzofuran-4-yl, isobenzofuran-5-yl, isobenzofuran-6-yl and isobenzofuran-7-yl. The term “benzothiophenyl” (also called benzo[b]thienyl) as used herein includes 2-benzo[b]thiophenyl, 3-benzo[b]thiophenyl, 4-benzo[b]thiophenyl, 5-benzo[b]thiophenyl, 6-benzo[b]thiophenyl and -7-benzo[b]thiophenyl (also called benzothien-2-yl, benzothien-3-yl, benzothien-4-yl, benzothien-5-yl, benzothien-6-yl and benzothien-7-yl). The term “isobenzothiophenyl” (also called benzo[c]thienyl) as used herein includes isobenzothien-1-yl, isobenzothien-3-yl, isobenzothien-4-yl, isobenzothien-5-yl, isobenzothien-6-yl and isobenzothien-7-yl. The term “indazolyl” (also called 1H-indazolyl or 2-azaindolyl) as used herein includes 1H-indazol-1-yl, 1H-indazol-3-yl, 1H-indazol-4-yl, 1H-indazol-5-yl, 1H-indazol-6-yl, 1H-indazol-7-yl, 2H-indazol-2-yl, 2H-indazol-3-yl, 2H-indazol-4-yl, 2H-indazol-5-yl, 2H-indazol-6-yl, and 2H-indazol-7-yl. The term “benzimidazolyl” as used herein includes benzimidazol-1-yl, benzimidazol-2-yl, benzimidazol-4-yl, benzimidazol-5-yl, benzimidazol-6-yl and benzimidazol-7-yl. The term “1,3-benzoxazolyl” as used herein includes 1,3-benzoxazol-2-yl, 1,3-benzoxazol-4-yl, 1,3-benzoxazol-5-yl, 1,3-benzoxazol-6-yl and 1,3-benzoxazol-7-yl. The term “1,2-benzisoxazolyl” as used herein includes 1,2-benzisoxazol-3-yl, 1,2-benzisoxazol-4-yl, 1,2-benzisoxazol-5-yl, 1,2-benzisoxazol-6-yl and 1,2-benzisoxazol-7-yl. The term “2,1-benzisoxazolyl” as used herein includes 2,1-benzisoxazol-3-yl, 2,1-benzisoxazol-4-yl, 2,1-benzisoxazol-5-yl, 2,1-benzisoxazol-6-yl and 2,1-benzisoxazol-7-yl. The term “1,3-benzothiazolyl” as used herein includes 1,3-benzothiazol-2-yl, 1,3-benzothiazol-4-yl, 1,3-benzothiazol-5-yl, 1,3-benzothiazol-6-yl and 1,3-benzothiazol-7-yl. The term “1,2-benzoisothiazolyl” as used herein includes 1,2-benzisothiazol-3-yl, 1,2-benzisothiazol-4-yl, 1,2-benzisothiazol-5-yl, 1,2-benzisothiazol-6-yl and 1,2-benzisothiazol-7-yl. The term “2,1-benzoisothiazolyl” as used herein includes 2,1-benzisothiazol-3-yl, 2,1-benzisothiazol-4-yl, 2,1-benzisothiazol-5-yl, 2,1-benzisothiazol-6-yl and 2,1-benzisothiazol-7-yl. The term “benzotriazolyl” as used herein includes benzotriazol-1-yl, benzotriazol-4-yl, benzotriazol-5-yl, benzotriazol-6-yl and benzotriazol-7-yl. The term “1,2,3-benzoxadiazolyl” as used herein includes 1,2,3-benzoxadiazol-4-yl, 1,2,3-benzoxadiazol-5-yl, 1,2,3-benzoxadiazol-6-yl and 1,2,3-benzoxadiazol-7-yl. The term “2,1,3-benzoxadiazolyl” as used herein includes 2,1,3-benzoxadiazol-4-yl, 2,1,3-benzoxadiazol-5-yl, 2,1,3-benzoxadiazol-6-yl and 2,1,3-benzoxadiazol-7-yl. The term “1,2,3-benzothiadiazolyl” as used herein includes 1,2,3-benzothiadiazol-4-yl, 1,2,3-benzothiadiazol-5-yl, 1,2,3-benzothiadiazol-6-yl and 1,2,3-benzothiadiazol-7-yl. The term “2,1,3-benzothiadiazolyl” as used herein includes 2,1,3-benzothiadiazol-4-yl, 2,1,3-benzothiadiazol-5-yl, 2,1,3-benzothiadiazol-6-yl and 2,1,3-benzothiadiazol-7-yl. The term “thienopyridinyl” as used herein includes thieno[2,3-b]pyridinyl, thieno[2,3-c]pyridinyl, thieno[3,2-c]pyridinyl and thieno[3,2-b]pyridinyl. The term “purinyl” as used herein includes purin-2-yl, purin-6-yl, purin-7-yl and purin-8-yl. The term “imidazo[1,2-a]pyridinyl”, as used herein includes imidazo[1,2-a]pyridin-2-yl, imidazo[1,2-a]pyridin-3-yl, imidazo[1,2-a]pyridin-4-yl, imidazo[1,2-a]pyridin-5-yl, imidazo[1,2-a]pyridin-6-yl and imidazo[1,2-a]pyridin-7-yl. The term “1,3-benzodioxolyl”, as used herein includes 1,3-benzodioxol-4-yl, 1,3-benzodioxol-5-yl, 1,3-benzodioxol-6-yl, and 1,3-benzodioxol-7-yl. The term “quinolinyl” as used herein includes quinolin-2-yl, quinolin-3-yl, quinolin-4-yl, quinolin-5-yl, quinolin-6-yl, quinolin-7-yl and quinolin-8-yl. The term “isoquinolinyl” as used herein includes isoquinolin-1-yl, isoquinolin-3-yl, isoquinolin-4-yl, isoquinolin-5-yl, isoquinolin-6-yl, isoquinolin-7-yl and isoquinolin-8-yl. The term “cinnolinyl” as used herein includes cinnolin-3-yl, cinnolin-4-yl, cinnolin-5-yl, cinnolin-6-yl, cinnolin-7-yl and cinnolin-8-yl. The term “quinazolinyl” as used herein includes quinazolin-2-yl, quinazolin-4-yl, quinazolin-5-yl, quinazolin-6-yl, quinazolin-7-yl and quinazolin-8-yl. The term “quinoxalinyl” as used herein includes quinoxalin-2-yl, quinoxalin-5-yl, and quinoxalin-6-yl.

The term “heterocyclyloxy”, as a group or part of a group, refers to a group having the formula —O—Rⁱ wherein Rⁱ is heterocyclyl as defined herein above.

The term “heterocyclylalkyloxy”, as a group or part of a group, refers to a group having the formula —O—R^{a}—Rⁱ wherein Rⁱ is heterocyclyl, and R^{a} is alkyl as defined herein above.

The term “heterocyclylalkyl”, as a group or part of a group, means an alkyl as defined herein, wherein at least one hydrogen atom is replaced by at least one heterocyclyl as defined herein. A non-limiting example of a heterocyclyl-alkyl group is 2-tetrahydrofuranyl-methyl.

The term “heteroaryloxy”, as a group or part of a group, refers to a group having the formula —O—R^{k} wherein R^{k} is heteroaryl as defined herein above.

The term “heteroarylalkyloxy”, as a group or part of a group, refers to a group having the formula —O—R^{a}—Rⁱ wherein Rⁱ is heteroaryl, and R^{a} is alkyl as defined herein above.

The term “heterocyclyl-alkyl” as a group or part of a group, refers to an alkyl in which one of the hydrogen atoms bonded to a carbon atom, typically a terminal or sp3 carbon atom, is replaced with a heterocyclyl. A non-limiting example of a heterocyclyl-alkyl group is 2-piperidinyl-methylene. The heterocyclyl-alkyl group can comprise 6 to 20 atoms, e.g. the alkyl moiety of the heterocycle-alkyl group is 1 to 6 carbon atoms and the heterocyclyl moiety is 5 to 14 atoms.

The term “heterocyclyl-alkenyl” as a group or part of a group refers to an alkenyl in which one of the hydrogen atoms bonded to a carbon atom, is replaced with an heterocyclyl. The heterocyclyl-alkenyl group can comprise 6 to 20 atoms, e.g. the alkenyl moiety of the heterocyclyl-alkenyl group is 1 to 6 carbon atoms and the heterocyclyl moiety is 5 to 14 atoms, such as ═CH—Rⁱ, wherein Rⁱ is heterocyclyl as defined herein above.

The term “heterocyclyl-heteroalkyl” as a group or part of a group refers to a heteroalkyl in which one of the hydrogen atoms bonded to a carbon atom, typically a terminal or sp3 carbon atom, is replaced with a heterocyclyl. The heterocyclyl-heteroalkyl group can comprise 6 to 20 atoms, e.g. the heteroalkyl moiety of the heterocyclyl-heteroalkyl group can comprise 1 to 6 carbon atoms and the heterocyclyl moiety can comprise 5 to 14 atoms. In some embodiments heterocyclyl-heteroalkyl is selected from the group comprising heterocyclyl-O-alkyl, heterocyclylalkyl-O-alkyl, heterocyclyl-NH-alkyl, heterocyclyl-N(alkyl)₂, heterocyclylalkyl-NH-alkyl, heterocyclylalkyl-N-(alkyl)₂, heterocyclyl-S-alkyl, and heterocyclylalkyl-S-alkyl.

The term “heterocyclyl-heteroalkenyl” as a group or part of a group refers to a heteroalkenyl in which one of the hydrogen atoms bonded to a carbon atom, is replaced with an heterocyclyl. The heterocyclyl-heteroalkenyl group can comprise 6 to 20 atoms, e.g. the heteroalkenyl moiety of the heterocyclyl-heteroalkenyl group can comprise 1 to 6 carbon atoms and the heterocyclyl moiety can comprise 5 to 14 atoms. In some embodiments heterocyclyl-heteroalkenyl is selected from the group comprising heterocyclyl-O-alkenyl, heterocyclylalkyl-O-alkenyl, heterocyclyl-NH-alkenyl, heterocyclyl-N(alkenyl)₂, heterocyclylalkyl-NH-alkenyl, heterocyclylalkyl-N-(alkenyl)₂, heterocyclyl-S-alkenyl, and heterocyclylalkenyl-S-alkenyl.

The term “heteroaryl-alkyl” as a group or part of a group refers to an alkyl in which one of the hydrogen atoms bonded to a carbon atom, typically a terminal or sp3 carbon atom, is replaced with a heteroaryl. An example of a heteroaryl-alkyl group is 2-pyridyl-methylene. The heteroaryl-alkyl group can comprise 6 to 20 atoms, e.g. the alkyl moiety of the heteroaryl-alkyl group can comprise 1 to 6 carbon atoms and the heteroaryl moiety can comprise 5 to 14 atoms.

The term “heteroaryl-alkenyl” as a group or part of a group refers to an alkenyl in which one of the hydrogen atoms bonded to a carbon atom, is replaced with an heteroaryl. The heteroaryl-alkenyl group can comprise 6 to 20 atoms, e.g. the alkenyl moiety of the heteroaryl-alkenyl group can comprise 1 to 6 carbon atoms and the heteroaryl moiety can comprise 5 to 14 atoms, such as ═CH—R^{k}, wherein R^{k} is heteroaryl as defined herein above.

The term “heteroaryl-heteroalkyl” as a group or part of a group as used herein refers to a heteroalkyl in which one of the hydrogen atoms bonded to a carbon atom, typically a terminal or sp3 carbon atom, is replaced with a heteroaryl. The heteroaryl-heteroalkyl group comprises 6 to 20 atoms, e.g. the heteroalkyl moiety of the heteroaryl-heteroalkyl group is 1 to 6 carbon atoms and the heteroaryl moiety is 5 to 14 atoms. In some embodiments heteroaryl-heteroalkyl is selected from the group comprising heteroaryl-O-alkyl, heteroarylalkyl-O-alkyl, heteroaryl-NH-alkyl, heteroaryl-N(alkyl)₂, heteroarylalkyl-NH-alkyl, heteroarylalkyl-N-(alkyl)₂, heteroaryl-S-alkyl, and heteroarylalkyl-S-alkyl.

The term “heteroaryl-heteroalkenyl” as a group or part of a group as used herein refers to a heteroalkenyl in which one of the hydrogen atoms bonded to a carbon atom, is replaced with an heteroaryl. The heteroaryl-heteroalkenyl group comprises 6 to 20 atoms, e.g. the heteroalkenyl moiety of the heteroaryl-heteroalkenyl group is 1 to 6 carbon atoms and the heteroaryl moiety is 5 to 14 atoms. In some embodiments heteroaryl-heteroalkenyl is selected from the group comprising heteroaryl-O-alkenyl, heteroarylalkenyl-O-alkenyl, heteroaryl-NH-alkenyl, heteroaryl-N(alkenyl)₂, heteroarylalkenyl-NH-alkenyl, heteroarylalkenyl-N-(alkenyl)₂, heteroaryl-S-alkenyl, and heteroarylalkenyl-S-alkenyl.

By way of example, carbon bonded heteroaryl or heterocyclic rings can be bonded at position 2, 3, 4, 5, or 6 of a pyridine, position 3, 4, 5, or 6 of a pyridazine, position 2, 4, 5, or 6 of a pyrimidine, position 2, 3, 5, or 6 of a pyrazine, position 2, 3, 4, or 5 of a furan, tetrahydrofuran, thiophene, pyrrole or tetrahydropyrrole, position 2, 4, or 5 of an oxazole, imidazole or thiazole, position 3, 4, or 5 of an isoxazole, pyrazole, or isothiazole, position 2 or 3 of an aziridine, position 2, 3, or 4 of an azetidine, position 2, 3, 4, 5, 6, 7, or 8 of a quinoline or position 1, 3, 4, 5, 6, 7, or 8 of an isoquinoline. Still more typically, carbon bonded heteroaryls and heterocyclyls include 2-pyridyl, 3-pyridyl, 4-pyridyl, 5-pyridyl, 6-pyridyl, 3-pyridazinyl, 4-pyridazinyl, 5-pyridazinyl, 6-pyridazinyl, 2-pyrimidinyl, 4-pyrimidinyl, 5-pyrimidinyl, 6-pyrimidinyl, 2-pyrazinyl, 3-pyrazinyl, 5-pyrazinyl, 6-pyrazinyl, 2-thiazolyl, 4-thiazolyl, or 5-thiazolyl. By way of example, nitrogen bonded heterocyclic rings are bonded at position 1 of an aziridine, azetidine, pyrrole, pyrrolidine, 2-pyrroline, 3-pyrroline, imidazole, imidazolidine, 2-imidazoline, 3-imidazoline, pyrazole, pyrazoline, 2-pyrazoline, 3-pyrazoline, piperidine, piperazine, indole, indoline, 1H-indazole, position 2 of a isoindole, or isoindoline, position 4 of a morpholine, and position 9 of a carbazole, or B-carboline. Still more typically, nitrogen bonded heteroaryls or heterocyclyls include 1-aziridyl, 1-azetedyl, 1-pyrrolyl, 1-imidazolyl, 1-pyrazolyl, and 1-piperidinyl.

As used herein and unless otherwise stated, the terms “alkoxy”, “cyclo-alkoxy”, “aryloxy”, “arylalkyloxy”, “heteroaryloxy” “heterocyclyloxy”, “alkylthio”, “cycloalkylthio”, “arylthio”, “arylalkylthio”, “heteroarylthio” and “heterocyclylthio” refer to substituents wherein an alkyl group, respectively a cycloalkyl, aryl, arylalkyl heteroaryl, or heterocyclyl (each of them such as defined herein), are attached to an oxygen atom or a sulfur atom through a single bond, such as but not limited to methoxy, ethoxy, propoxy, butoxy, thioethyl, thiomethyl, phenyloxy, benzyloxy, mercaptobenzyl and the like. The same definitions will apply for alkenyl and alkynyl instead of alkyl.

The term “alkylthio”, as a group or part of a group, refers to a group having the formula —S—R^{b} wherein R^{b} is alkyl as defined herein above. Non-limiting examples of alkylthio groups include methylthio (—SCH₃), ethylthio (—SCH₂CH₃), n-propylthio, isopropylthio, n-butylthio, isobutylthio, sec-butylthio, tert-butylthio and the like.

The term “alkenylthio”, as a group or part of a group, refers to a group having the formula —S—R^{d} wherein R^{d} is alkenyl as defined herein above.

The term “arylthio”, as a group or part of a group, refers to a group having the formula —S—R^{g} wherein R^{g} is aryl as defined herein above.

The term “arylalkylthio”, as a group or part of a group, refers to a group having the formula —S—R^{a}—R^{g} wherein R^{a} is alkylene and R^{g} is aryl as defined herein above.

The term “heterocyclylthio”, as a group or part of a group, refers to a group having the formula —S—Rⁱ wherein Rⁱ is heterocyclyl as defined herein above.

The term “heteroarylthio”, as a group or part of a group, refers to a group having the formula —S—R^{k} wherein R^{k} is heteroaryl as defined herein above.

The term “heterocyclylalkylthio”, as a group or part of a group, refers to a group having the formula —S—R^{a}—Rⁱ wherein R^{a} is alkylene and Rⁱ is heterocyclyl as defined herein above.

The term “heteroarylalkylthio”, as a group or part of a group, refers to a group having the formula —S—R^{a}—R^{k} wherein R^{a} is alkylene and R^{k} is heteroaryl as defined herein above.

The term “alkyl-SO2”, as a group or part of a group, refers to a group having the formula —SO2-R^{b} wherein R^{b} is alkyl as defined herein above. Non-limiting examples of alkyl-SO2 groups include methyl-SO2, ethyl-SO2 and the like.

The term “heteroalkyl-SO2”, as a group or part of a group, refers to a group having the formula —SO2-R^{e} wherein R^{e} is heteroalkyl as defined herein above.

The term “aryl-SO2”, as a group or part of a group, refers to a group having the formula —SO2-R^{g} wherein R^{g} is aryl as defined herein above.

The term “heteroaryl-SO2”, as a group or part of a group, refers to a group having the formula —SO2-R^{k} wherein R^{k} is heteroaryl as defined herein above.

The term “heterocyclyl-SO2”, as a group or part of a group, refers to a group having the formula —SO2-Rⁱ wherein Rⁱ is heterocyclyl as defined herein above.

The term “mono- or di-alkylamino”, as a group or part of a group, refers to a group of formula —N(R^{o})(R^{b}) wherein R^{o} is hydrogen, or alkyl, R^{b} is alkyl. Thus, alkylamino include mono-alkyl amino group (e.g. mono-alkylamino group such as methylamino and ethylamino), and di-alkylamino group (e.g. di-alkylamino group such as dimethylamino and diethylamino). Non-limiting examples of suitable mono- or di-alkylamino groups include n-propylamino, isopropylamino, n-butylamino, i-butylamino, sec-butylamino, t-butylamino, pentylamino, n-hexylamino, di-n-propylamino, di-i-propylamino, ethylmethylamino, methyl-n-propylamino, methyl-i-propylamino, n-butylmethylamino, i-butylmethylamino, t-butylmethylamino, ethyl-n-propylamino, ethyl-i-propylamino, n-butylethylamino, i-butylethylamino, t-butylethylamino, di-n-butylamino, di-i-butylamino, methylpentylamino, methylhexylamino, ethylpentylamino, ethylhexylamino, propylpentylamino, propylhexylamino, and the like.

The term “mono- or di-arylamino”, as a group or part of a group, refers to a group of formula —N(R^{q})(R^{r}) wherein R^{q} and R^{r} are each independently selected from hydrogen, aryl, or alkyl, wherein at least one of R^{q} or R^{r} is aryl.

The term “mono- or di-heteroarylamino”, as a group or part of a group, refers to a group of formula —N(R^{u})(R^{v}) wherein R^{u} and R^{v} are each independently selected from hydrogen, heteroaryl, or alkyl, wherein at least one of R^{u} or R^{v} is heteroaryl as defined herein.

The term “mono- or di-alkylamino-SO2”, as a group or part of a group, refers to a group of formula —SO2-N(R^{o})(R^{b}) wherein R^{o} is hydrogen, or alkyl, R^{b} is alkyl. “alkylamino” includes mono-alkyl amino group (e.g. mono-alkylamino group such as methylamino and ethylamino), and di-alkylamino group (e.g. di-alkylamino group such as dimethylamino and diethylamino).

The term “mono- or di-arylamino-SO2”, as a group or part of a group, refers to a group of formula —SO2-N(R^{q})(R^{r}) wherein R^{q} and R^{r} are each independently selected from hydrogen, aryl, or alkyl, wherein at least one of R^{q} or R^{r} is aryl.

The term “mono- or di-heteroarylamino-SO2”, as a group or part of a group, refers to a group of formula —SO2-N(R^{u})(R^{v}) wherein R^{u} and R^{v} are each independently selected from hydrogen, heteroaryl, or alkyl, wherein at least one of R^{u} or R^{v} is heteroaryl as defined herein.

The term “mono- or di-heterocyclylamino”, as a group or part of a group, refers to a group of formula —N(R^{w})(R^{x}) wherein R^{w} and R^{x} are each independently selected from hydrogen, heterocyclyl, or alkyl, wherein at least one of R^{w} or R^{x} is heterocyclyl as defined herein.

As used herein and unless otherwise stated, the term halogen means any atom selected from the group consisting of fluorine (F), chlorine (Cl), bromine (Br) and iodine (I).

The terminology regarding a chemical group “which optionally includes one or more heteroatoms, said heteroatoms being selected from the atoms consisting of O, S, and N” as used herein, refers to a group where one or more carbon atoms are replaced by an oxygen, nitrogen or sulphur atom and thus includes, depending on the group to which is referred, heteroalkyl, heteroalkenyl, heteroalkynyl, heteroalkyl, heteroalkenyl, heteroalkynyl, cycloheteroalkyl, cycloheteroalkenyl, cycloheteroalkynyl, heteroaryl, arylheteroalkyl, heteroarylalkyl, heteroarylheteroalkyl, arylheteroalkenyl, heteroarylalkenyl, heteroarylheteroalkenyl, heteroarylheteroalkenyl, arylheteroalkynyl, heteroarylalkynyl, heteroarylheteroalkynyl, among others. This term therefore comprises, depending on the group to which is referred, as an example alkoxy, alkenyloxy, alkynyloxy, alkyl-O-alkylene, alkenyl-O-alkylene, arylalkoxy, benzyloxy, heteroaryl-heteroalkyl, heterocyclyl-heteroalkyl, heteroaryl-alkoxy, heterocyclyl-alkoxy, among others. As an example, the terminology “alkyl which optionally includes one or more heteroatoms, said heteroatoms being selected from the atoms consisting of O, S, and N” therefore refers to heteroalkyl, meaning an alkyl which comprises one or more heteroatoms in the hydrocarbon chain, whereas the heteroatoms may be positioned at the beginning of the hydrocarbon chain, in the hydrocarbon chain or at the end of the hydrocarbon chain. Examples of heteroalkyl include methoxy, methylthio, ethoxy, propoxy, CH₃—O—CH₂—, CH₃—S—CH₂—, CH₃—CH₂—O—CH₂—, CH₃—NH—, (CH₃)₂—N—, (CH₃)₂—CH₂—NH—CH₂—CH₂—, among many other examples. As an example, the terminology “arylalkylene which optionally includes one or more heteroatoms in the alkylene chain, said heteroatoms being selected from the atoms consisting of O, S, and N” therefore refers to arylheteroalkylene, meaning an arylalkylene which comprises one or more heteroatoms in the hydrocarbon chain, whereas the heteroatoms may be positioned at the beginning of the hydrocarbon chain, in the hydrocarbon chain or at the end of the hydrocarbon chain. “Arylheteroalkylene” thus includes aryloxy, arylalkoxy, aryl-alkyl-NH— and the like and examples are phenyloxy, benzyloxy, aryl-CH₂—S—CH₂—, aryl-CH₂—O—CH₂—, aryl-NH—CH₂— among many other examples. The same counts for “heteroalkenylene”, and other terms used herein when referred to “which optionally includes one or more heteroatoms, said heteroatoms being selected from the atoms consisting of O, S, and N”.

The term “single bond” as used herein for a linking group i.e. in a way that a certain linking group is selected from a single bond, etc. in the formulas herein, refers to a molecule wherein the linking group is not present and therefore refers to compounds with a direct linkage via a single bond between the two moieties being linked by the linking group.

As used herein with respect to a substituting group, and unless otherwise stated, the terms “substituted” such as in “substituted alkyl”, “substituted alkenyl”, substituted alkynyl”, “substituted aryl”, “substituted heteroaryl”, “substituted heterocyclyl”, “substituted arylalkyl”, “substituted heteroaryl-alkyl”, “substituted heterocyclyl-alkyl” and the like refer to the chemical structures defined herein, and wherein the said alkyl, alkenyl, alkynyl, group and/or the said aryl, heteroaryl, or heterocyclyl may be optionally substituted with one or more substituents (preferable 1, 2, 3, 4, 5 or 6), meaning that one or more hydrogen atoms are each independently replaced with at least one substituent. Typical substituents include, but are not limited to and in a particular embodiment said substituents are being independently selected from the group consisting of halogen, amino, hydroxyl, sulfhydryl, alkyl, alkoxy, alkenyl, alkenyloxy, alkynyl, alkynyloxy, cycloalkyl, cycloalkenyl, cycloalkynyl, heteroalkyl, heteroalkenyl, heteroalkynyl, aryl, heteroaryl, heterocyclyl, arylalkyl, arylalkenyl, arylalkynyl, cycloalkyl-alkyl, cycloalkylalkenyl, cycloalkylalkynyl, heteroaryl-alkyl, heterocyclyl-alkyl, heteroaryl-alkenyl, heterocyclyl-alkenyl and heteroaryl-alkynyl, heterocyclyl-alkynyl, —X, -Z, —O⁻, —OZ, ═O, —SZ, —S⁻, ═S, —NZ₂, —N⁺Z₃, ═NZ, ═N—OZ, —CX₃ (e.g. trifluoromethyl), —CN, —OCN, —SCN, —N═C═O, —N═C═S, —NO, —NO₂, ═N₂, —N₃, —NZC(O)Z, —NZC(S)Z, —NZC(O)O⁻, —NZC(O)OZ, —NZC(S)OZ, —NZC(O)NZZ, NZC(NZ)Z, NZC(NZ)NZZ, —C(O)NZZ, —C(NZ)Z, —S(O)₂O⁻, —S(O)₂OZ, —S(O)₂Z, —OS(O)₂OZ, —OS(O)₂Z, —OS(O)₂O⁻, —S(O)₂NZ, —S(O)Z, —OP(O)(OZ)₂, —P(O)(OZ)₂, —P(O)(O⁻)₂, —P(O)(OZ)(O⁻), —P(O)(OH)₂, —C(O)Z, —C(O)X, —C(S)Z, —C(O)OZ, —C(O)O⁻, —C(S)OZ, —C(O)SZ, —C(S)SZ, —C(O)NZZ, —C(S)NZZ, —C(NZ)NZZ, —OC(O)Z, —OC(S)Z, —OC(O)O⁻, —OC(O)OZ, —OC(S)OZ, wherein each X is independently a halogen selected from F, Cl, Br, or I; and each Z is independently —H, alkyl, alkenyl, alkynyl, heteroalkyl, heteroalkenyl, heteroalkynyl, aryl, heteroaryl, heterocyclyl, cycloalkyl, protecting group or prodrug moiety, while two Z bonded to a nitrogen atom can be taken together with the nitrogen atom to which they are bonded to form a heteroaryl, or heterocyclyl. Alkyl(ene), alkenyl(ene), and alkynyl(ene) groups may also be similarly substituted.

Any substituent designation that is found in more than one site in a compound of this invention shall be independently selected.

Substituents optionally are designated with or without bonds. Regardless of bond indications, if a substituent is polyvalent (based on its position in the structure referred to), then any and all possible orientations of the substituent are intended.

The term “heteroatom(s)” as used herein means an atom selected from nitrogen, which can be quaternized; oxygen; and sulfur, including sulfoxide and sulfone.

The term “hydroxyl” as used herein means —OH.

The term “carbonyl” as used herein means carbon atom bonded to oxygen with a double bond, i.e., C═O.

The term “amino” as used herein means the —NH₂ group.

The term “imino” as used herein means the ═NH group.

The term “alkyl-imino”, as a group or part of a group, refers to a group having the formula ═N—R^{b} wherein R^{b} is alkyl as defined herein above.

The term “aryl-imino”, as a group or part of a group, refers to a group having the formula ═N—R^{g} wherein R^{g} is aryl as defined herein above.

The term “carboxyl” as used herein means —C(O)OH.

The term “hydroxycarbonylalkyl” as a group or part of a group, refers to a group having the formula —R^{b}—C(O)OH wherein R^{b} is alkyl as defined herein above. Non-limiting example of a hydroxycarbonylalkyl group includes e.g. hydroxycarbonylmethyl.

The term “hydroxycarbonylalkenyl” as a group or part of a group, refers to a group having the formula —R^{d}—C(O)OH wherein R^{d} is alkenyl as defined herein above. Non-limiting example of a hydroxycarbonylalkenyl group includes e.g hydroxycarbonylmethylene, hydroxycarbonylpropylene.

The compounds as defined herein can be prepared while using a series of chemical reactions well known to those skilled in the art. The compounds of interest having a structure according to the general formula (I), or general formula (II), or general formula (III), or general formula (IV), and all other formulas described herein and embodiments thereof can be prepared using a series of chemical reactions well known to those skilled in the art.

As used herein and unless otherwise stated, the term “enantiomer” means each individual optically active form of a compound as defined herein, having an optical purity or enantiomeric excess (as determined by methods standard in the art) of at least 80% (e.g. at least 90% of one enantiomer and at most 10% of the other enantiomer), preferably at least 90% and more preferably at least 98%.

The term “isomers” as used herein means all possible isomeric forms, including tautomeric and stereochemical forms, which the compounds of formulae herein may possess, but not including position isomers. Typically, the structures shown herein exemplify only one tautomeric or resonance form of the compounds, but the corresponding alternative configurations are contemplated as well. Unless otherwise stated, the chemical designation of compounds denotes the mixture of all possible stereochemically isomeric forms, said mixtures containing all diastereomers and enantiomers (since the compounds of formulae herein may have at least one chiral center) of the basic molecular structure, as well as the stereochemically pure or enriched compounds. More particularly, stereogenic centers may have either the R- or S-configuration, and multiple bonds may have either cis- or trans-configuration.

Pure isomeric forms of the said compounds are defined as isomers substantially free of other enantiomeric or diastereomeric forms of the same basic molecular structure. In particular, the term “stereoisomerically pure” or “chirally pure” relates to compounds having a stereoisomeric excess of at least about 80% (e.g. at least 90% of one isomer and at most 10% of the other possible isomers), preferably at least 90%, more preferably at least 94% and most preferably at least 97%. The terms “enantiomerically pure” and “diastereomerically pure” should be understood in a similar way, having regard to the enantiomeric excess, respectively the diastereomeric excess, of the mixture in question.

Separation of stereoisomers is accomplished by standard methods known to those skilled in the art. One enantiomer of a compound as defined herein can be separated substantially free of its opposing enantiomer by a method such as formation of diastereomers using optically active resolving agents. Separation of isomers in a mixture can be accomplished by any suitable method well known to those skilled in the art, including: (1) formation of ionic, diastereomeric salts with chiral compounds and separation by fractional crystallization or other methods, (2) formation of diastereomeric compounds with chiral derivatizing reagents, separation of the diastereomers, and conversion to the pure enantiomers, or (3) enantiomers can be separated directly under chiral conditions. Under method (1), diastereomeric salts can be formed by reaction of enantiomerically pure chiral bases such as brucine, quinine, ephedrine, strychnine, a-methyl-b-phenylethylamine (amphetamine), and the like with asymmetric compounds bearing acidic functionality, such as carboxylic acid and sulfonic acid. The diastereomeric salts may be induced to separate by fractional crystallization or ionic chromatography. For separation of the optical isomers of amino compounds, addition of chiral carboxylic or sulfonic acids, such as camphorsulfonic acid, tartaric acid, mandelic acid, or lactic acid can result in formation of the diastereomeric salts. Alternatively, by method (2), the substrate to be resolved may be reacted with one enantiomer of a chiral compound to form a diastereomeric pair. Diastereomeric compounds can be formed by reacting asymmetric compounds with enantiomerically pure chiral derivatizing reagents, such as menthyl derivatives, followed by separation of the diastereomers and hydrolysis to yield the free, enantiomerically enriched compound. A method of determining optical purity involves making chiral esters, such as a menthyl ester or Mosher ester, a-methoxy-a-(trifluoromethyl)phenyl acetate (Jacob III. (1982) J. Org. Chem. 47:4165), of the racemic mixture, and analyzing the NMR spectrum for the presence of the two atropisomeric diastereomers. Stable diastereomers can be separated and isolated by normal- and reverse-phase chromatography following methods for separation of atropisomeric naphthyl-isoquinolines (see e.g. WO 96/15111). Under method (3), a racemic mixture of two asymmetric enantiomers is separated by chromatography using a chiral stationary phase. Suitable chiral stationary phases are, for example, polysaccharides, in particular cellulose or amylose derivatives. Appropriate eluents or mobile phases for use in combination with said polysaccharide chiral stationary phases are hexane and the like, modified with an alcohol such as ethanol, isopropanol and the like.

The terms cis and trans are used herein in accordance with Chemical Abstracts nomenclature and include reference to the position of the substituents on a ring moiety. The absolute stereochemical configuration of the compounds of the formulae described herein may easily be determined by those skilled in the art while using well-known methods such as, for example, X-ray diffraction.

Also included within the scope of this invention are salts of compounds as defined herein with one or more amino acids, especially the naturally-occurring amino acids found as protein components. The amino acid typically is one bearing a side chain with a basic or acidic group, e.g., lysine, arginine or glutamic acid, or a neutral group such as glycine, serine, threonine, alanine, isoleucine, or leucine.

The term “pharmaceutically acceptable salts” as used herein means the therapeutically active non-toxic salt forms which the compounds as defined herein are able to form. Therefore, the compounds of this invention optionally comprise salts of the compounds herein, especially pharmaceutically acceptable non-toxic salts containing, for example, Na⁺, Li⁺, K⁺, Ca²⁺ and Mg²⁺. Such salts may include those derived by combination of appropriate cations such as alkali and alkaline earth metal ions or ammonium and quaternary amino ions with an acid anion moiety, typically a carboxylic acid. The compounds as defined herein may bear multiple positive or negative charges. The net charge of the compounds as defined herein may be either positive or negative. Any associated counter ions are typically dictated by the synthesis and/or isolation methods by which the compounds are obtained. Typical counter ions include, but are not limited to ammonium, sodium, potassium, lithium, halides, acetate, trifluoroacetate, etc., and mixtures thereof. It will be understood that the identity of any associated counter ion is not a critical feature as defined herein, and that the invention encompasses the compounds in association with any type of counter ion. Moreover, as the compounds can exist in a variety of different forms, the invention is intended to encompass not only forms of the compounds that are in association with counter ions (e.g., dry salts), but also forms that are not in association with counter ions (e.g., aqueous or organic solutions). Metal salts typically are prepared by reacting the metal hydroxide with a compound of this invention. Examples of metal salts which are prepared in this way are salts containing Li⁺, Na⁺, and K⁺. A less soluble metal salt can be precipitated from the solution of a more soluble salt by addition of the suitable metal compound. In addition, salts may be formed from acid addition of certain organic and inorganic acids to basic centers, typically amines, or to acidic groups. Examples of such appropriate acids include, for instance, inorganic acids such as hydrohalogen acids, e.g. hydrochloric or hydrobromic acid, sulfuric acid, nitric acid, phosphoric acid and the like; or organic acids such as, for example, acetic, propanoic, hydroxyacetic, 2-hydroxypropanoic, 2-oxopropanoic, lactic, pyruvic, oxalic (i.e. ethanedioic), malonic, succinic (i.e. butanedioic acid), maleic, fumaric, malic, tartaric, citric, methanesulfonic, ethanesulfonic, benzenesulfonic, p-toluenesulfonic, cyclohexanesulfamic, salicylic (i.e. 2-hydroxybenzoic), p-aminosalicylic and the like. Furthermore, this term also includes the solvates which the compounds of formulae herein as well as their salts are able to form, such as for example hydrates, alcoholates and the like. Finally, it is to be understood that the compositions herein comprise compounds as defined herein in their unionized, as well as zwitterionic form, and combinations with stoichiometric amounts of water as in hydrates.

The compounds as defined herein also include physiologically acceptable salts thereof. Examples of physiologically acceptable salts of the compounds as defined herein include salts derived from an appropriate base, such as an alkali metal (for example, sodium), an alkaline earth (for example, magnesium), ammonium and NX₄⁺ (wherein X is C₁-C₄ alkyl). Physiologically acceptable salts of an hydrogen atom or an amino group include salts of organic carboxylic acids such as acetic, benzoic, lactic, fumaric, tartaric, maleic, malonic, malic, isethionic, lactobionic and succinic acids; organic sulfonic acids, such as methanesulfonic, ethanesulfonic, benzenesulfonic and p-toluenesulfonic acids; and inorganic acids, such as hydrochloric, sulfuric, phosphoric and sulfamic acids. Physiologically acceptable salts of a compound containing a hydroxy group include the anion of said compound in combination with a suitable cation such as Na⁺ and NX₄⁺ (wherein X typically is independently selected from H or a C₁-C₄ alkyl group). However, salts of acids or bases which are not physiologically acceptable may also find use, for example, in the preparation or purification of a physiologically acceptable compound. All salts, whether or not derived from a physiologically acceptable acid or base, are within the scope of the present invention.

Another embodiment of this invention relates to “pro-drug” forms of the compounds as defined herein. It may be desirable to formulate the compounds of the present invention in the form of a chemical species which itself is not significantly biologically-active, but which when delivered to the animal, mammal or human will undergo a chemical reaction catalyzed by the normal function of the body, said chemical reaction having the effect of releasing a compound as defined herein. The term “pro-drug” thus relates to these species which are converted in vivo into the active pharmaceutical ingredient. For the purpose of the present invention the term “prodrug”, as used herein, relates to an inactive or significantly less active derivative of a compound such as represented by the structural formulae herein described, which undergoes spontaneous or enzymatic transformation within the body in order to release the pharmacologically active form of the compound.

In certain embodiments, the present invention relates to a compound of formula (I), or of formula (II) or of formula (III) or of formula (IV) as defined herein, or as represented in Tables A to F, for use as a medicament. In certain embodiments, the present invention relates to a compound of formula (I), or of formula (II) or of formula (III) or of formula (IV) as defined herein, or as represented in Tables A to F, for use as a modulator of Rel hydrolase and/or synthetase activity. In certain embodiments, the present invention relates to a compound of formula (I), or of formula (II) or of formula (III) or of formula (IV) as defined herein, for use as an inhibitor of the Rel hydrolase and/or synthetase activity or as represented in Tables A to F,. In certain embodiments, the present invention relates to a compound of formula (I), or of formula (II) or of formula (III) or of formula (IV) as defined herein, or as represented in any of Tables A to F, for use as an activator of Rel hydrolase and/or synthetase activity. In certain embodiments, the present invention relates to a compound of formula (I), or of formula (II) or of formula (III) or of formula (IV) as defined herein, or as represented in any of Tables A to F, for use as an effector of the Rel hydrolase and/or synthetase activity.

Compounds identified by the methods as described herein such as the herein mentioned compounds of general formula (I), or (II), or (III) or (IV) may be included in a pharmaceutical formulation. Techniques regarding the formulation and administration of pharmaceutical compositions are known to a skilled person and have been described in the art (e.g. the reference book: Remington: The Science and Practice of Pharmacy, periodically revised).

In certain embodiments, the present invention relates to a compound of formula (I), or of formula (II) or of formula (III), or of formula (IV) as defined herein, or as represented in any of Tables A to F, for use in treating infections with antibiotic (multi)resistant bacteria. In certain embodiments, the present invention relates to a compound of formula (I), or of formula (II) or of formula (III) or of formula (IV) as defined herein, or as represented in any of Tables A to F, for use in treating infections with dormant, latent or persistent bacteria.

Therefore, the invention further relates to a method of treating or preventing infections with antibiotic (multi)resistant bacteria in a subject comprising a Rel modulator as described in any embodiment herein, or a pharmaceutical composition comprising a Rel modulator as described herein. A skilled person appreciates that in order to be effective, the Rel modulator has to be administered in a therapeutically effective amount to achieve a biological or medical response in a subject. Method and practices to determine therapeutically effective doses of a pharmaceutical active ingredient, in the context of the current specification the Rel modulator are known to a person skilled in the art. Evidently, the required dosage or amount that is needed to arrive at a therapeutically effective dose needs to be determined on a case-by-case and subject-to-subject basis. It is standard practice to adapt a dosage to a certain individual to obtain an optimal, i.e. ideal effect or response. Optimally, a considerable number of distinct parameters need to be assessed when determining an optimal dosage, or dosage schedule and include but are by no means limited to the nature and degree of the disease to be treated, gender of the subject, subject age, body weight, other medical indications, nutrition, mode of administration, metabolic state, interference or influence of efficacy by other pharmaceutically active ingredients, etc. Furthermore each antibiotic (multi)resistant bacteria or bacterial infection may have a certain intrinsic degree of responsiveness to the used Rel modulator. The pharmaceutical compositions as referred to herein may comprise at least one additional pharmaceutical active ingredient. In certain embodiments, the pharmaceutical formulation further comprises one or more non-active pharmaceutical ingredients or inactive ingredients, also known in the art as excipients. Furthermore, the formulation may comprise pharmaceutically acceptable auxiliary substances as required to approximate physiological conditions, such as pH adjusting and buffering agents, preservatives, complexing agents, tonicity adjusting agents, wetting agents and the like.

Terms such as “subject”, “patient”, or “individual” may be used interchangeably herein and refer to animals, preferably warm-blooded animals, more preferably vertebrates, and even more preferably mammals. Preferred subjects are human subjects (Homo sapiens) including all genders and all age categories thereof. Adult subjects, elder subjects, newborn subjects, and foetuses are intended to be covered by the term “subject”.

The terms “treatment” or “treat” indicate the therapeutic treatment of an already developed disease or condition, such as the therapy of an (multi)resistant bacterial infection. Beneficial or desired clinical results may include, without limitation, alleviation of one or more symptoms or one or more biological markers, diminishment of extent of disease, stabilized (i.e., not worsening) state of disease, delay or slowing of disease progression, amelioration or palliation of the disease state, and the like. While the methods, uses, and modulators described herein act as a new class of therapeutics for (multi)resistant bacteria, it is evident that they may also be applied on bacteria or bacterial infections that do not show any antibiotic resistance, or only a limited degree of antibiotic resistance.

In certain embodiments, the Rel modulator used in a method of treatment is a Rel hydrolase and/or Rel synthetase inhibitor. In alternative embodiments, the Rel modulator used in the method of treatment is a Rel hydrolase and/or Rel synthetase activator. In certain embodiments, the method is directed to treatment of bacterial infections characterized by the presence of dormant, latent, or persistent bacteria. Also intended is treatment of a subject for treating or preventing infections with antibiotic (multi)resistant bacteria comprising at least two distinct Rel modulators as described herein. In certain embodiments, the Rel modulators as disclosed herein are used in conjunction with other distinct antibacterial molecules or compositions known in the art. In certain embodiments, the methods of treatment disclosed herein comprise use of at least one Rel modulator as disclosed herein and a distinct traditional antibacterial molecule or composition known in the art. In further embodiments, the traditional antibacterial molecule or composition acts on the ribosomal machinery of the bacteria. In certain further embodiments, the Rel modulator and the traditional antibacterial molecule or composition are used at distinct time points in therapy. In further embodiments, the traditional antibacterial molecule or composition and Rel modulator are used in an alternating manner. In certain embodiments, the subject is a subject diagnosed with a (multi)resistant bacterial infection. In certain embodiments, the bacterial infection is characterized by biofilm formation and/or deposition in said subject.

In certain aspects, the invention relates to the use of a Rel modulator as described herein, or a pharmaceutical composition comprising a Rel modulator as described in herein, for the manufacture of a medicament for the prevention or treatment of an antibiotic (multi)resistant bacterial infection. In certain embodiments, the use of a Rel modulator as described herein, or a pharmaceutical composition comprising a Rel modulator as described herein for the manufacture of a medicament for modulating the function of Rel is intended. Preferably, the activity of Rel is modulated by said Rel modulator or pharmaceutical composition in such a way that Rel hydrolase and/or Rel synthetase activity is inhibited or reduced. In alternative preferred embodiments, the activity of Rel is modulated by said Rel modulator or pharmaceutical composition in such a way that Rel hydrolase and/or Rel synthetase activity is upregulated. Furthermore, the use of a Rel modulator as disclosed herein for the manufacture of a medicament for the prevention or treatment of infections with dormant, latent, or persistent bacteria is also envisaged. Also intended is the use of a Rel modulator as described herein for the manufacture of a medicament for the prevention or treatment of infections characterized by biofilm formation.

In certain embodiments, the modulator of Rel as described herein is an inhibitor or Rel hydrolase and/or synthetase activity. In certain embodiments, the modulator of Rel is an inhibitor of Rel synthetase activity. In certain embodiments, the modulator of Rel is an effector of Rel hydrolase and/or synthetase activity. In certain embodiments, the modulator of Rel is an activator of Rel hydrolase activity. In certain embodiments, the modulator of Rel is an activator of Rel synthetase activity. In certain embodiments, the Rel modulator increases or decreases the Rel hydrolase activity with at least 30%, preferably at least 50%, at least 75%, most preferably at least 100% compared to Rel hydrolase activity in absence of said Rel modulator. In certain embodiments, the Rel modulator increases or decreases the Rel synthetase activity with at least 30%, preferably at least 50%, at least 75%, at least 100% compared to Rel synthetase activity in absence of said Rel modulator. In embodiments where the Rel modulator increases or upregulates Rel hydrolase or synthetase activity, said activity is upregulated by at least 1.5-fold, at least 2-fold, at least 3-fold, at least 5-fold, at least 10-fold, or more, when compared to the hydrolase or synthetase activity of Rel in identical conditions in absence of the Rel modulator.

“Effector” as used herein indicates a molecule which increases or decreases the activity of an enzyme by binding to the enzyme at a regulatory site which is optionally distinct from the catalytic site that binds the substrate. Hence, a skilled person appreciates that an effector molecule is a molecule that regulates the biological activity of a target protein by binding to said protein. Preferably, an effector is a “small” molecule as defined herein.

Therefore, a further aspect of the invention is directed to Rel modulators as described herein for use in treating infections with antibiotic (multi)resistant bacteria. In certain embodiments, the (multi)resistant bacteria of the bacterial infection are Gram-negative bacteria, Gram-positive bacteria, or a combination thereof. Non-limiting examples of (multi)resistant bacteria are found in Staphylococci, Enterococci, Gonococci, Streptococci, Salmonella, Mycobacteria, and numerous Gram-negative bacteria. In certain embodiments, the antibiotic (multi)resistant bacteria further are resistant to bacteriophages. By means of illustration and not limitation, specific examples of (multi)resistant bacteria are multiresistant tuberculosis bacterial strains, Common multidrug-resistant organisms are usually bacteria part of the following group of bacteria: Vancomycin-Resistant Enterococci, Methicillin-Resistant Staphylococcus aureus, Extended-spectrum β-lactamase producing Gram-negative bacteria, Klebsiella pneumoniae carbapenemase (KPC) producing Gram-negatives, Multidrug-Resistant Gram Negative (MDR GN) bacteria such as Enterobacter species, E. coli, Klebsiella pneumoniae, Acinetobacter baumannii, Pseudomonas aeruginosa. In certain embodiments, the bacterial infection is an infection caused by a bacteria or a combination of bacteria commonly annotated in the art as the ESKAPE group of bacteria (Boucher et al., Bad buds, no drugs: no ESKAPE! An update from the Infectious Diseases Society of America, Clinical infectious diseases, 2009). It is understood that the term “ESKAPE group” refers to a group of bacteria comprising Enterococcus faecium, Staphylococcus aureus, Klebsiella pneumoniae, Acinetobacter baumannii, Pseudomonas aeruginosa and Enterobacter species. A skilled person appreciates that the collection of bacterial strains and species that are annotated in the art as antibiotic (multi)resistant bacteria will evolve and likely expand over time, and that these organisms are also envisaged in this specification. It is evident that the Rel modulators as described herein are suitable for use in any bacterial infection that expresses a Rel protein as described herein.

Means and method to detect drug resistance and classify drug resistant bacteria have been described in the art and are therefore known to a skilled person (Fluit et al., Molecular detection of antimicrobial resistance, Clinical microbiology reviews, 2001).

In certain embodiments, (a portion of) antibiotic (multi)resistant bacteria in bacterial infections disclosed herein are dormant, latent, or persistent bacteria. In certain embodiments, a portion of the antibiotic (multi)resistant bacteria in bacterial are a combination of dormant, latent, persistent bacteria. In certain embodiments, a portion of the antibiotic (multi)resistant bacteria are metabolically active, i.e. displaying a normal metabolic state represented by a standard growth rate, and a distinct portion of said bacteria are characterized by a metabolically attenuated, or metabolically reduced, or metabolically inactive state. The terms dormant, latent and persistent are well known and characterized in detail in the art (e.g. in Cohen et al., Microbial persistence and the road to drug resistance, Cell host and microbe, 2013). It is evident that a bacterium may change one or more of its properties changing its state and is therefore to be classified as a dormant, latent or persistent bacterium at a later point in time, or vice versa no longer be classified as dormant, latent, or persistent bacterium.

Another aspect of the invention is directed to the use of the crystal structure of the Rel polypeptide as defined by the atomic coordinates presented in any one of Tables 1 to 4, or a subset thereof, or atomic coordinates which deviate from those in any one of Tables 1 to 4, or a subset thereof, by RMSD over protein backbone atoms by no more than 3 Å for designing and/or identifying a compound which modulates Rel hydrolase and/or synthetase activity. In certain embodiments, the use is directed to the design and/or identification of compounds which inhibit Rel hydrolase and/or Rel synthetase activity. In alternative embodiments, the use is directed to the design and/or identification of compounds which upregulate or enhance Rel hydrolase and/or Rel synthetase activity. In certain embodiments, the use is directed to designing and/or identifying allosteric Rel modulators. In certain embodiments, the use is directed to designing and/or identifying Rel modulators that bind to two distinct domain or regions of the Rel protein.

A different aspect of the invention regards a computer system comprising a database containing the atomic coordinates, or a subset thereof as defined in any one of Tables 1 to 4, stored on a computer readable storage medium, and a user interface to view the information. Also intended are data processing apparatuses, devices, and systems comprising a database containing the atomic coordinates, or a subset thereof as defined in any one of Tables 1 to 4, stored on a computer readable storage medium, and a user interface to view the information. Models and atomic coordinates as disclosed herein are typically stored on a machine-readable, or computer-readable medium which are known in the art and include as non-limiting examples magnetic or optical media and random-access or read-only memory, including tapes, diskettes, hard disks, CD-ROMs and DVDs, flash drives or chips, servers and the internet. In certain embodiments, the computer system comprises means for carrying out the methods as described herein. In certain embodiments, the computer system further comprises an input device to receive instructions from an operator. In certain embodiments, the computer system comprises and/or is connected to a remote data storage system, wherein the remote data storage system is located at a geographic location different from the location of the user interface to view the information. Said data storage system may be located in a network storage medium such as the internet, providing remote accessibility. In certain embodiments, the database comprised in the computer system is encrypted. In certain embodiments, the computer system has access to at least one database of compound structures, and a user can by appropriately instructing said computer system access said at least one database of compound structures. In certain embodiments, the compound, list of compounds, or compound database (also known as compound library) is loaded into the computer system by the operator. In alternative embodiments, the compound, list of compounds, or compound database is accessible by the computer system from a medium different than said computer system. In certain embodiments, the computer system comprises a processing unit to assess the degree of fit between any compound molecule loaded into the computer system and Rel. Also intended is a computer-readable storage medium comprising instructions which, when executed by a computer, causes the computer to carry out any one of the methods disclosed herein.

A further aspect relates to the use of a computer system as described herein for designing and/or identifying a compound (ligand) which modulates Rel activity. In certain embodiments, the use of said computer system is achieved by user input commands. In certain embodiments, the computer system comprises means to select candidate Rel modulators from a list of compounds, or a compound library. In certain embodiments, the computer system comprises means to select (a) candidate compound(s) and proposing structural changes to the at least one candidate compound to further increase the number of energetically favorable interactions between said compound and Rel and/or means to select (a) candidate compound(s) and proposing structural changes to the at least one candidate compound to reduce or eliminate structural interference between said candidate modulator and one or more Residues of Rel defined by the atomic coordinates in any one of Tables 1 to 4. When using a computer system as described herein, the user searching for Rel modulators, which may or may not be the operator of the computer is provided by an optionally printed list of candidate Rel modulator. The computer system provides the user with one or more candidate Rel modulators. In certain embodiments, the computer system can be used to only provide the uses with candidate compounds that inhibit Rel hydrolase and/or synthetase activity. In alternative embodiments, the computer system can be used to only provide the user with candidate compounds that upregulate Rel hydrolase and/or synthetase activity. In alternative embodiments, the computer system is used for designing and/or identifying an allosteric Rel modulator. In certain embodiments, the computer system is used to provide a visual representation, i.e. an image of the three-dimensional structure of Rel, optionally during interaction with the candidate Rel compound. In certain embodiments, a list of candidate Rel modulators is generated and stored, optionally sorted according to a scoring system as described herein, in an electronic file.

Further intended herein are the crystals comprising a Rel protein in one or more of its three-dimensional conformations. In a certain embodiment, a crystal of Rel in its unbound resting state is intended, comprising a structure characterized by the atomic coordinates or a subset thereof as defined in Table 1. It is understood that “unbound resting state” indicates the conformation the Rel protein adopts in absence of binding any Rel modulator or Rel substrate. In an alternative embodiment, a crystal of Rel in its synthetase active form is intended, comprising a structure characterized by the atomic coordinates or a subset thereof as defined in Table 2. In an alternative embodiment, a crystal structure of Rel in its hydrolase active form is intended, comprising a structure characterized by the atomic coordinates or a subset thereof as defined in Table 3. In yet an alternative embodiment, a crystal structure of Rel in its allosteric state is intended, comprising a structure characterized by the atomic coordinates or a subset thereof as defined in Table 4. The precise composition of the Rel crystals envisaged herein will depend on the method used to generate said crystal, and the prevalence of these different compositions are expected by a skilled person.

Any crystal structure disclosed herein is said to be characterized by, or conform to, or substantially conform to, a set or subset of atomic coordinates when a structure, or a substantial fragment of a structure has or falls within the limit RMSD value as disclosed herein. In a certain embodiment, at least 75%, preferably at least 80%, more preferably at least 90% of the crystal structure has the recited RMSD value. In certain embodiments, “substantially conform to” further refers to atoms of amino acid side chains. In this context, common amino acid side chains are side chains that are common between the structure substantially conform to a structure with particular atomic coordinates and structures being defined by said atomic coordinates of Tables 1, 2, 3, or 4.

Another aspect of the invention relates to a method for producing a medicament, pharmaceutical composition or drug, the process comprising providing a compound as described herein and preparing a medicament, pharmaceutical composition or drug containing said compound. In certain embodiments, the pharmaceutical composition is formulated into a unit dosage form, including but not limited to hard capsules, soft capsules, tablets, coated tablets such as lacquered tablets or sugar-coated tablets, granules, aqueous or oily solutions, syrups, emulsions, suspensions, ointments, pastes, lotions, gels, inhalants or suppositories. In certain embodiments, the pharmaceutical composition is administered systemically, however in alternative embodiments the pharmaceutical composition is administered locally. In further embodiments, the pharmaceutical composition is suitable for oral, rectal, bronchial, nasal, topical, buccal, sublingual, transdermal, vaginal or parenteral administration, or in a form suitable for administration by inhalation. In certain embodiments, depending on the specific administration route said pharmaceutical composition is suited for, the process further comprises addition of ingredients not considered an active pharmaceutical ingredient to improve administration of the pharmaceutical composition. The unit dosage form comprising the pharmaceutical composition may be characterized by an immediate release pattern, a delayed release pattern, or a sustained release pattern, which are each terms standardly used in the technical field of pharmacy and have been defined in Pharmacopeias published by government authorities or medical or pharmaceutical societies. A skilled person appreciates that these bodies of work are reference works in the field of pharmacy.

A further aspect of the invention is directed to a computer system, intended to generate three dimensional structural representations of a Rel enzyme, Rel enzyme homologues or analogues, complexes of Rel enzyme with binding compounds or modulators, or complexes of Rel enzyme homologues or analogues with compounds or modulators, or, to analyze or optimize binding of compounds or modulators to said Rel enzyme or homologues or analogues, or complexes thereof, the system containing computer-readable data comprising one or more of:

(a) the coordinates of the Rel enzyme structure, listed in any one of Tables 1 to 4, optionally varied by a root mean square deviation of residue backbone atoms of not more than 3 Å, or selected coordinates thereof;

(b) the coordinates of a Rel enzyme homologue or analogue generated by homology modeling of the target based on the data in (a);

(c) the coordinates of a candidate binding compound or modulator generated by interpreting X-ray crystallographic data or NMR data by reference to the coordinates of the Rel enzyme structure, listed in any one of Tables 1 to 4, optionally varied by a root mean square deviation of residue backbone atoms of not more than 3 Å, or selected coordinates thereof, and

(d) structure factor data derivable from the coordinates of (a), (b) or (c).

In certain embodiments, the computer system comprises data comprising any combination of (a), (b), (c), or (d). In further embodiments, the user is able to adjust, remove, or add further data to the computer system. In certain embodiments, the computer system is able to receive additional data, adjust data, or remove data pertaining to (a), (b), (c), or (d). In certain embodiments, the user is able to access synthesis protocols of compounds or modulators through the computer system. In certain embodiments, the computer system directs the user to a synthesis protocol.

The term ‘homolog’ used herein indicates a pair of genes, in the context of the invention a gene encoding Rel that are said or evidenced to have a shared ancestry. Homology as used in the art is typically indicative for a similar nucleotide sequence, or a nucleotide sequence encoding at least a similar amino acid sequence for both genes. A further sub classification of homology can be established which is then commonly based on orthology and paralogy. Genes are said to be orthologous if they share a common ancestral sequence and have been diverging from each other by at least one speciation event. Thus, orthologs arise when a species diverges into two separate species. In contrast, paralogous genes are genes that arise through duplication events in the last common ancestor of the species under investigation. “Analog” as used herein refers to at least two genes each present in distinct taxa that do not share a common ancestor but nevertheless have the same function, or share at least one common function. Sequence similarity of the gene or the gene product is not a prerequisite for two genes to be analogs.

A different aspect of the invention relates to a computer-readable storage medium, comprising a data storage material encoded with computer readable data, wherein the data comprises one or more of

(a) the coordinates of the Rel enzyme structure, listed in any one of Tables 1 to 4, optionally varied by a root mean square deviation of residue backbone atoms of not more than 3 Å, or selected coordinates thereof;

(b) the coordinates of a Rel enzyme homologue or analogue generated by homology modeling of the target based on the data in (a);

(c) the coordinates of a candidate binding compound or modulator generated by interpreting X-ray crystallographic data or NMR data by reference to the coordinates of the Rel enzyme structure, listed in any one of Tables 1 to 4, optionally varied by a root mean square deviation of residue backbone atoms of not more than 3 Å, or selected coordinates thereof, and

(d) structure factor data derivable from the coordinates of (a), (b) or (c).

In certain embodiments, the computer readable data is encrypted and requires authentication or authorization credentials from a user or second computer-readable storage system for a computer system to be able to access said data. In certain embodiments, the computer-readable storage medium is a physical storage medium. In alternative embodiments, the computer-readable storage medium is a non-physical storage medium or a storage medium perceived to be a non-physical storage medium (i.e. a cloud based storage medium).

In a different aspect the invention relates to a computer-readable storage medium comprising a data storage material encoded with a first set of computer-readable data comprising a Fourier transform of at least a portion of the structural coordinates of the Rel enzyme listed in any one of Tables 1 to 4, optionally varied by a root mean square deviation of residue backbone atoms of not more than 3 Å, or selected coordinates thereof; which data, when combined with a second set of machine readable data comprising an X-ray diffraction pattern of a molecule or molecular complex of unknown structure, using a machine programmed with the instructions for using said first set of data and said second set of data, can determine at least a portion of the structure coordinates corresponding to the second set of machine readable data. Fourier transformation in the context of the invention is to be interpreted as the application of a molecular-replacement approach.

As envisaged herein by the term “Fourier transform”, the three-dimensional transformation of a molecular model is calculated in a first step. Subsequently, the weighed reciprocal lattice is rotated according to the calculated transformation. Fourier transformation in molecular biology, and more specifically structure biology, has been described in the art (Rabinovich et al., Molecular replacement: the revival of the molecular Fourier transform method, Acta crystallographica section D biological crystallography, 1998). In certain embodiments, the X-ray diffraction pattern of a molecule or molecular complex of unknown structure is obtained by an apparatus operably coupled to said computer storage medium. In alternative embodiments, the X-ray diffraction pattern of a molecule or molecular complex of unknown structure is inputted to said computer-readable storage medium by user instructions. In yet alternative embodiments, the X-ray diffraction pattern of a molecule or molecular complex of unknown structure is retrieved by a computer system comprising the computer-readable storage medium from a public (accessible) database.

In certain embodiments, the computer system or computer-readable storage medium as described herein further comprises a database containing information on the three dimensional structure of candidate compounds or modulators which are small molecules. In certain embodiments, the computer system or computer-readable storage medium further comprises a means to retrieve information from public information databases on the three dimensional structure of candidate compounds or modulators, which are “small” molecules as defined herein, including the non-limiting examples of PubChem (https://pubchem.ncbi.nlm.nih.gov), the Zinc database (https://www.zinc.docking.org), and/or MolPort (https://www.molport.com). In certain embodiments, the computer system further generates information indicating which list or subset of atomic coordinates of any one of Tables 1, 2, 3, or 4 shows or is predicted to show the highest number of energetically favorable interactions with any candidate modulator assessed by said computer system. In certain embodiments the user receives an automatically generated list of candidate compounds ranked according to the number of energetically favorable interactions with the Rel protein as defined by each list or subset of atomic coordinates of any one of Tables 1 to 4. In further embodiments the computer system provides the user with a number of common structural groups any combination of candidate modulator may be differentiated by.

While the invention has been described in conjunction with specific embodiments thereof, it is evident that many alternatives, modifications, and variations will be apparent to those skilled in the art in light of the foregoing description. Accordingly, it is intended to embrace all such alternatives, modifications, and variations as follows in the spirit and broad scope of the appended claims. The herein disclosed aspects and embodiments of the invention are further supported by the following non-limiting examples.

### EXAMPLES

1. Structure Analysis of Rel Enzyme with Catalytically Engaged Synthetase or Nuclease Domains.

The paradigm of RSH catalysis is based on the structure of N-terminal region of S. dysgalactiae Rel (Rel_{Seq}^{NTD}) solved more than a decade ago. Rel_{Seq}^{NTD} is formed by two catalytic domains with opposing activities—ppGpp hydrolase (HD) and ppGpp synthetase (SYN) (Hogg et al., Conformational antagonism between opposing active sites in a bifunctional RelA/SpoT homolog modulates (p)ppGpp metabolism during the stringent response Cell, 2004). Serendipitously, the two Rel_{Seq}^{NTD} molecules observed in the same crystal lattice were locked in contrasting conformations leading to the hypothesis of reciprocal regulation of SYN and HD domains in archetypical RSH enzymes. However, the enzyme contained a nucleotide bound in each active site in one of the conformations and only one active site occupied in the other one. This contradicts earlier observations that suggested catalysis was incompatible with the simultaneous activation of synthetase and hydrolase function (Avarbock et al., Cloning and characterization of a bifunctional RelA/SpoT homologue from Mycobacterium tuberculosis, Gene, 1999, and Mechold et al., Differential regulation by ppGpp versus pppGpp in Escheria coli, Nucleic acids research, 2016). Thus, to directly test this hypothesis it is essential to solve the structures of Rel enzymes with catalytically engaged SYN or HD domains.

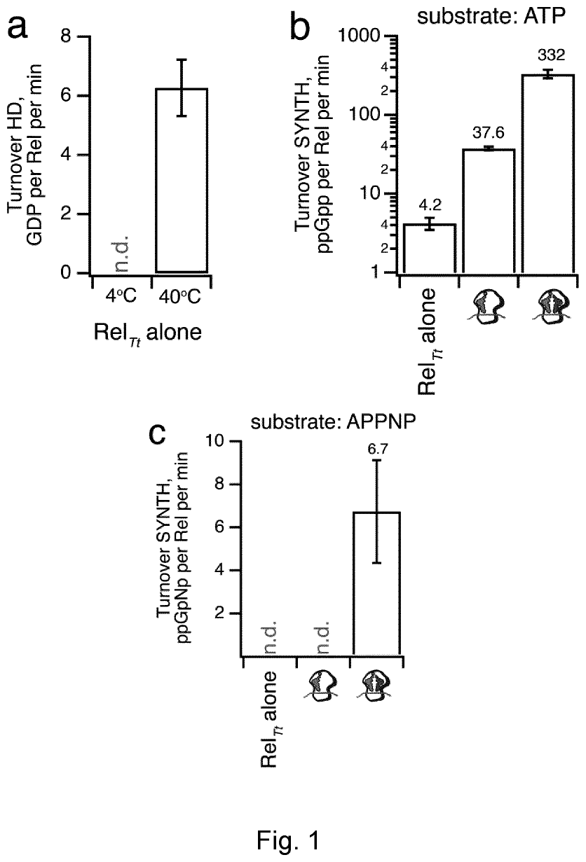

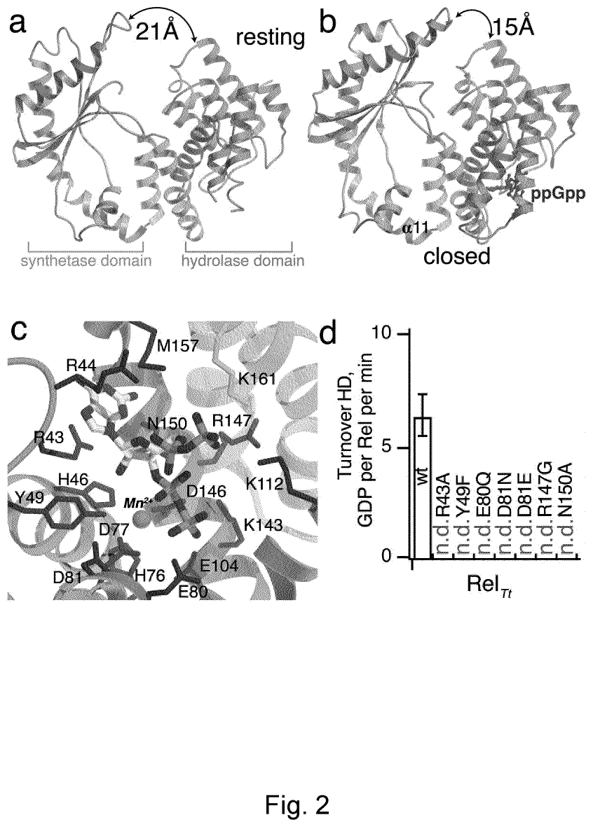

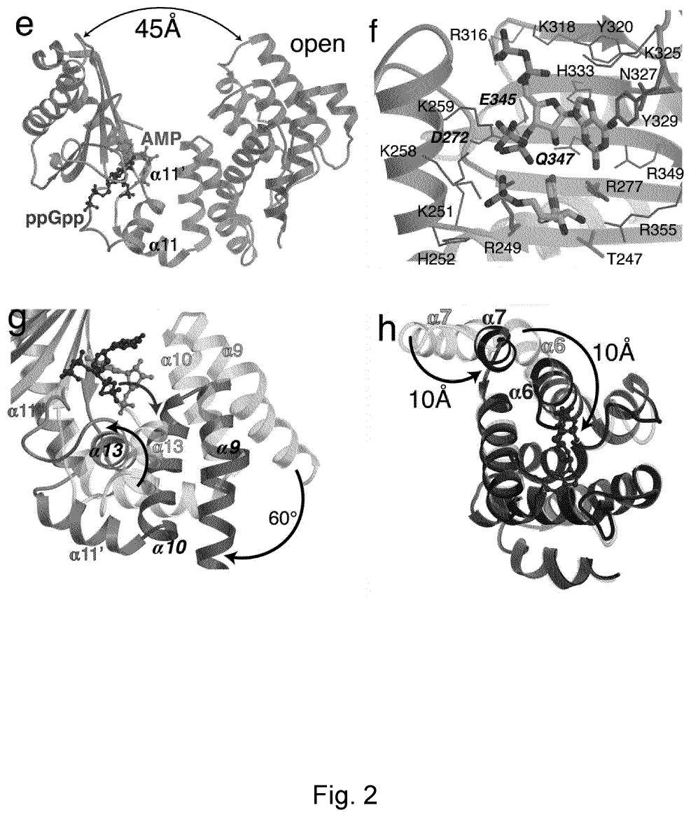

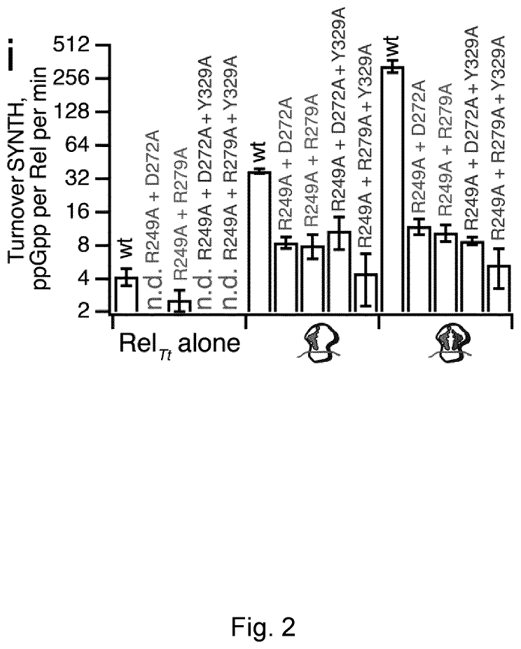

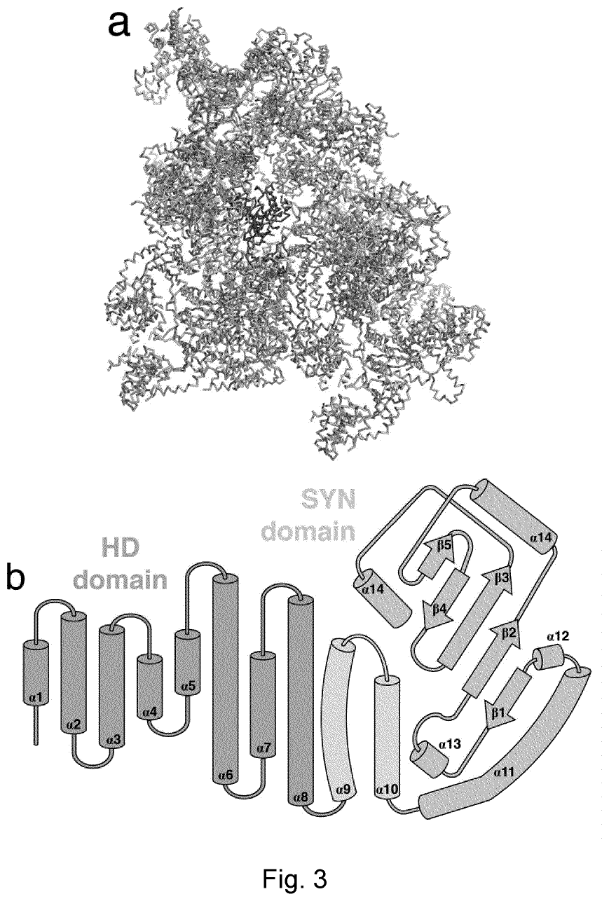

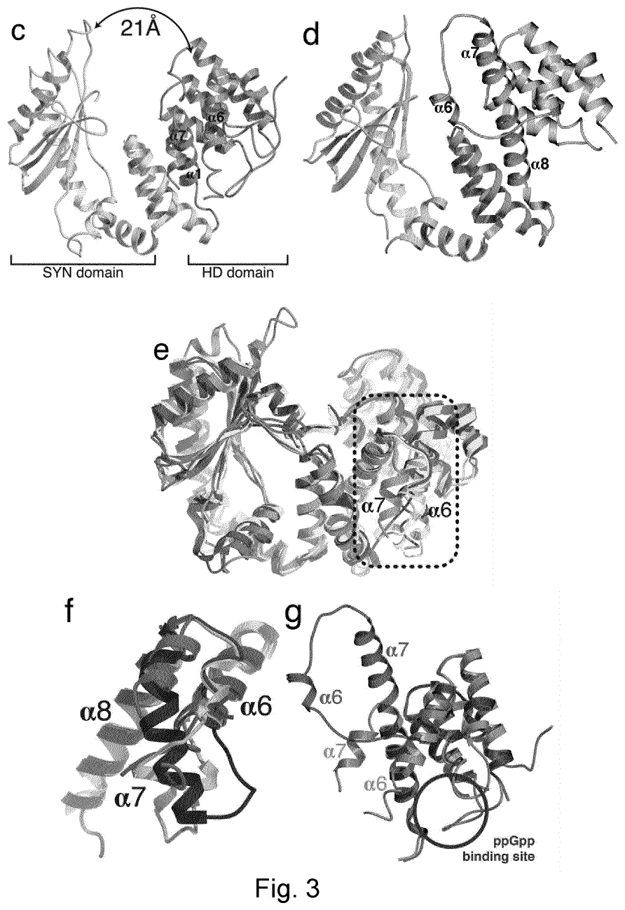

To understand how nucleotide binding stimulates the enzymatic capacity of RSH enzymes, we took advantage of T. thermophilus Rel NTD (Rel_{Tt}^{NTD}, amino acid positions 1-355) as an experimental system. Rel_{Tt}^{NTD} hydrolysis activity is virtually undetectable at 4° C. (FIG. 1a and Table 5), which is not surprising given that T. thermophilus has an optimal growth temperature of about 65° C. This enabled co-crystallization in the presence of the native ppGpp substrate. To generate the structure of Rel_{Tt}^{NTD} engaged in ppGpp synthesis, we used APPNP, a β-γ phosphate non hydrolysable ATP analogue, that reacts exceedingly slowly with GDP in the active center of Rel_{Tt}^{NTD} yielding ppGpNp (FIG. 1b-c and Table 5). The structure of unbound Rel_{Tt}^{NTD} (FIG. 2a and FIG. 3a-d) recapitulates the structures of Rel_{Seq}^{NTD}, M. tuberculosis Rel (RelA_{Mtb}^{NTD}) as well as the mono-functional synthetase-only E. coli RSH RelA (RelA_{Ec}) (Singal et al., Crystallographic and solution structure of the N-terminal domain of the Rel protein from Mycobacterium tuberculosis, FEBS, 2017). Only the conformation of α-helices α6, α7 and loop α6-α7 are noticeably different (FIG. 3c-d). In RelA_{Ec} the α6-α7 loop is projected towards the pseudo-hydrolase site of RelA_{Ec}, effectively blocking the site, whereas in Rel_{Tt}^{NTD}, RelA_{Mtb}^{NTD} and Rel_{Seq}^{NTD} α6-α7 is partially disordered and pointing in an opposite direction (Hogg et al., Conformational antagonism between opposing active sites in a bifunctional RelA/SpoT homolog modulates (p)ppGpp metabolism during the stringent response [corrected], Cell, 2004, Arenz et al., The stringent factor RelA adopts an open conformation on the ribosome to stimulate ppGpp synthesis, Nucleic acids research, 2016, Brown et al., Ribosome-dependent activation of stringent control, Nature 2016, and Loveland et al., Ribosome*RelA structures reveal the mechanism of stringent response activation, Elife, 2016) (FIG. 3e-f). Nucleotide-free Rel_{Tt}^{NTD} contains two different conformations in the same crystal lattice with a similar arrangement of both catalytic domains. The major difference between both conformations is that the α6-α7 motif of the hydrolase domain is observed projected completely away from the active site with α-helix α6 largely unwound in an extended conformation (FIG. 3c-d and 3f). Although this extreme conformation is primarily stabilized by the lattice contacts and likely to be a result of the crystallization process, it underscores the highly dynamic nature of the hydrolase domain, in particular the α6-α7 motif and its importance for catalysis.

TABLE 5
ppGpp synthesis and hydrolysis reaction parameters.
Each measurement was performed in triplicate.

| | Synthase activity | | | | | | Hydrolase |
|---|---|---|---|---|---|---|---|
| | Substrate: GDP + ATP | | | Substrate: GDP + APPNP | | | activity |
| | | | Rel_{Tt} + | | | Rel_{Tt} + | Substrate: |
| | | Rel_{Tt} + | IC + | | Rel_{Tt} + | IC + | ppGpp |
| | Rel_{Tt} | IC | tRNA^{Val} | Rel_{Tt} | IC | tRNA^{Val} | Rel_{Tt} |
| Rel_{Tt} | 4.2 ± 0.7 | 37.7 ± 1.9 | 332.3 ± 41.7 | N.D. | N.D. | 6.7 ± 2.4 | 6.3 ± 1.0 |
| R249A | N.D. | 8.5 ± 1.0 | 12.0 ± 2.0 | — | — | — | — |
| D272A | | | | | | | |
| R249A | 2.6 ± 0.6 | 8.6 ± 2 | 10.5 ± 1.8 | — | — | — | — |
| R277A | | | | | | | |
| R249A | N.D. | 10.9 ± 3.6 | 8.8 ± 0.69 | — | — | — | — |
| D272A | | | | | | | |
| Y329A | | | | | | | |
| R249A | N.D. | 4.5 ± 2.3 | 5.4 ± 2.1 | — | — | — | — |
| R277A | | | | | | | |
| Y329A | | | | | | | |
| R43A | — | — | — | — | — | — | N.D. |
| Y49F | — | — | — | — | — | — | N.D. |
| E80Q | — | — | — | — | — | — | N.D. |
| D81N | — | — | — | — | — | — | N.D. |
| D81E | — | — | — | — | — | — | N.D. |
| R147G | — | — | — | — | — | — | N.D. |
| N150A | — | — | — | — | — | — | N.D. |

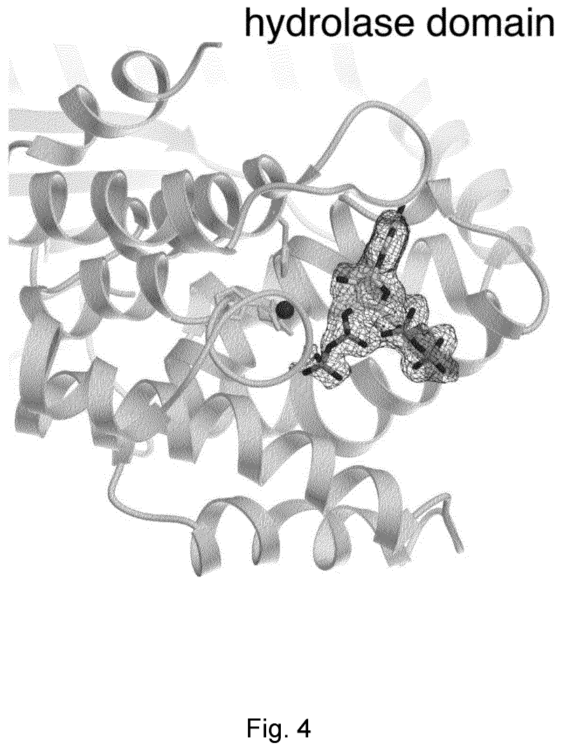

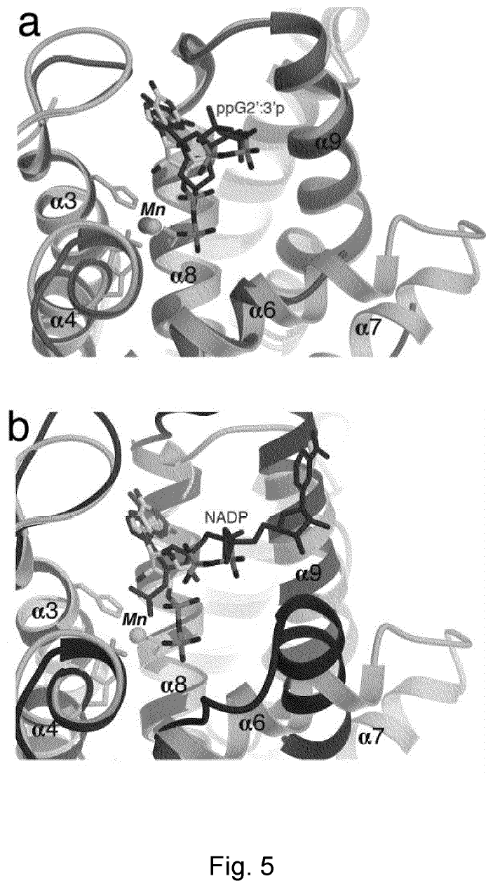

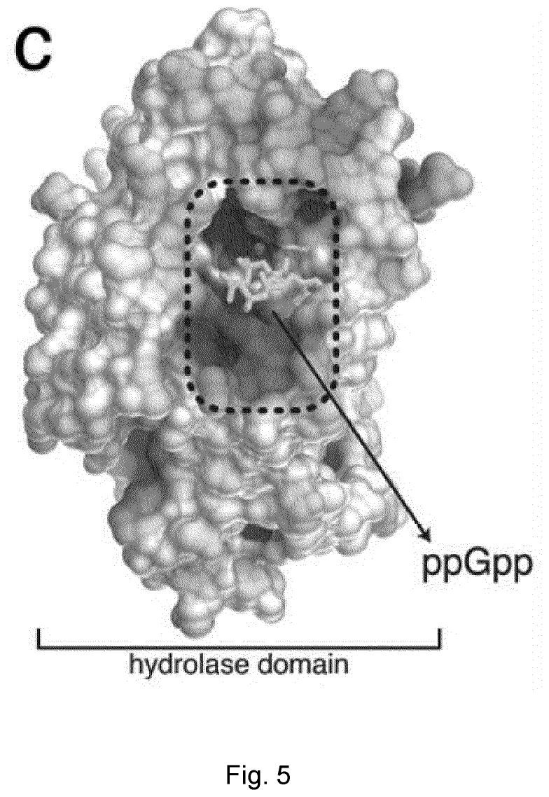

From the structure of the Rel_{Tt}^{NTD}-ppGpp complex (FIG. 2b and FIG. 4), it is immediately apparent that the complex is in a more compacted conformation than the resting state of Rel_{Tt}^{NTD} diverging from all other known conformation of Rel and RelA enzymes (FIGS. 2a and 2b). In the complex, ppGpp makes a large number of contacts with the enzyme (FIG. 2c) and is bound in a conformation reminiscent of that of ppG2′:3′p in the active site of Rel_{Seq}^{NTD} and NADP bound to a single-domain small alarmone hydrolase (SAH) RSH hMesh1 (Sun et al., A metazoan ortholog of SpoT hydrolyzes ppGpp and functions in starvation responses. Nature structural and molecular biology, 2010). (PDBID 5VXA) (FIG. 5a-b). The guanosine base is stacked between R43, R44 and M157 and makes hydrogen-bonds with S45, N150 and T153, while the ribose makes a van der Waals interaction with N150 and a hydrogen bond with Y49. The 2′- and 3′ oxygen atoms from the ribose are held very close (within 4.5 Å) to the Mn2+ ion and N150, which suggests an essential role for the metal ion in the deprotonation of the scissile bond and subsequent stabilization of a nucleophilic water molecule (FIG. 2c).

2. Characterization of Conformational Rearrangements During Substrate Binding.

The hydrolase active site has a remarkable distribution of surface electrostatics. The site consists of a deep and wide cavity with one half of the site positively charged and involved in the stabilization of the 5′ poly-phosphate groups of the substrate and the other predominately acidic and more directly involved in the 3′-pyrophosphate hydrolysis (FIG. 5c). The 3′-pyrophosphate group of ppGpp is bound in nearly the same position as that of ppG2′:3′p (FIG. 5a) in the acidic half of the active site formed by D77, E80, D81, E104 and D146 which is crucial for catalysis. This acidic section of the active site has already been highlighted as a functional hot-spot (Hogg et al., Conformational antagonism between opposing active sites in a bifunctional RelA/SpoT homolog modulates (p)ppGpp metabolism during the stringent response [corrected], Cell, 2004). Substitutions in the conserved HDX₃ED motif (Aravind et al., The HD domain defines a new superfamily of metal dependent phosphohydrolases. Trends in biochemical sciences, 1998) (GD or EV) completely abrogate hydrolysis in Rel_{Seq}^{NTD6} and the motif has diverged to ₈₂FPLADA₈₇ in the inactive hydrolase domain of E. coli RelA suggesting this could be one of the factors contributing to the lack of activity of the specialised E. coli synthetase. Indeed, conservative substitutions of E80 (E80Q) and D81 (D81N) or even changes in the distance of the carboxylate group relative to ppGpp in the HD domain (as in D81E) have a strong effect on the activity of the enzyme (FIG. 2d).

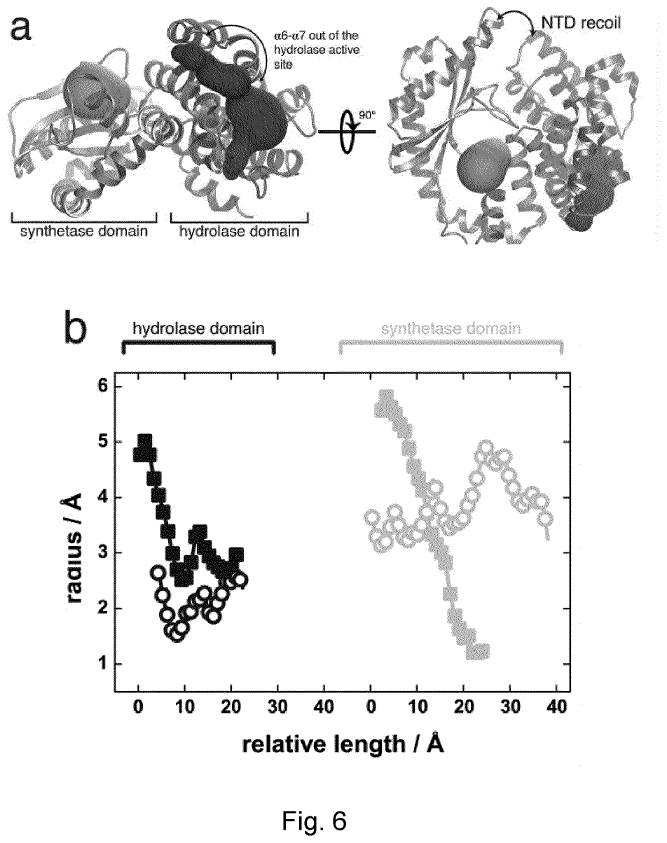

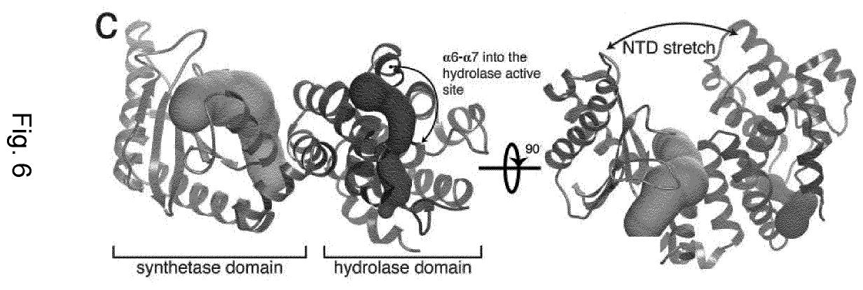

The 5′-pyrophosphate group of ppGpp is stabilized by the damping effect of K112, K143, R147 and K161 and projects towards the α-helix α6 (FIG. 2c). From this binding mode we propose that the relative spatial arrangement between α6 and α9 would determine the specificity of the hydrolase function. Indeed, as observed in the Rel_{Seq}^{NTD}-ppG2′:3′p and hMesh1-NADP complexes, it is the local disposition of α6 and α9 what allows accommodation of the 5′-pyrophosphate and nicotinamide riboside groups (FIG. 5a-b). As with the case of the acidic patch, substitutions at this positive site (R147G), towards the phosphate binding region also strongly affect hydrolysis (FIG. 2d). The other crucial observation from the Rel_{Tt}^{NTD}-ppGpp complex is that the entire SYN domain moves 6 Å closer to the HD domain compared to the resting state, sterically occluding and switching off the synthetase active site (FIG. 2a-b, FIG. 6a-b). In addition, the α6-α7 loop becomes fully structured and moves around 85° away from the position occupied by α6-α7 in RelA_{Ec} in the pseudo-active site of the HD domain. This movement of α6-α7 aligns together all the residues that constitute the aforementioned positive patch and allows the binding ppGpp in the active site. As a result, the hydrolase site of the enzyme becomes fully accessible in contrast with the synthetase site that is even more confined (FIG. 6a-b).

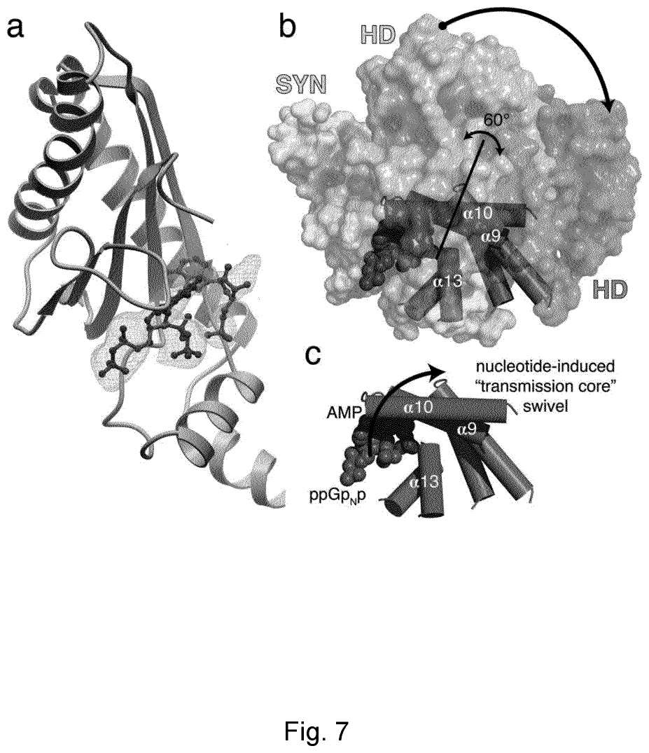

To understand how substrates control the ppGpp synthesis by the SYN domain, we solved the structure of Rel_{Tt}^{NTD} in a post-catalytic (PC) state (FIG. 2e) in complex with the reaction products AMP and ppGp_{N}p (FIG. 2f and FIG. 7). The structure of Rel_{Tt}^{NTD} in this PC conformation (FIG. 2e) shows a remarkably contrasting picture with the Rel_{Tt}^{NTD} ppGpp complex (FIG. 2b). The presence of two nucleotides in the synthetase site is accompanied by major rearrangements that stretch both domains almost 45 Å apart (FIG. 2e). The comparison of the two opposing conformations of the two active catalytic states suggests a potential allosteric signal transduction route (FIG. 2g). The central 3-α-helix bundle (C3HB) motif of the Rel_{Seq}^{NTD} enzyme forms a small hydrophobic core with α13 of the SYN domain that connects both catalytic domains. The wedging effect of the nucleotides is thus spread towards the HD domain via the α13-C3HB ‘transmission’ core that has swivelled orthogonally to the α9 dipole approximately 60° (FIG. 2g and FIG. 7b-c). This fractures α11 in two (α11′ and α11″), exposing the SYN domain active site and stretching the HD domain away. This conformational rearrangement portrays a much bigger allosteric effect associated with the switch to an active synthetase state than that expected from the two conformations of Rel_{Seq}^{NTD} which involved a rotation of 10° stretching both domains around 2.4 Å apart. Considering the lattice constraints and the partial occupancy of nucleotides in both active sites of Rel_{Seq}^{NTD}, it is not surprising that the enzyme is observed in an intermediate state closer to the resting state than to either active catalytic conformation. In the HD domain, these rearrangements are accompanied by a conformational change in loop α6-α7 that approaches towards the catalytic residues of the hydrolase center (FIG. 2h). These structural changes effectively close the hydrolase site, reducing its radius to almost half its size and expanding the radius and dimensions of synthetase active site in order to accommodate both nucleotide substrates (FIG. 7b-c). From the structure of the free Rel_{Tt}^{NTD} and Rel_{Seq}^{NTD} bound to GDP, it is clear that only the presence of both nucleotides in the active site of the synthetase domain can trigger such large domain rearrangements.

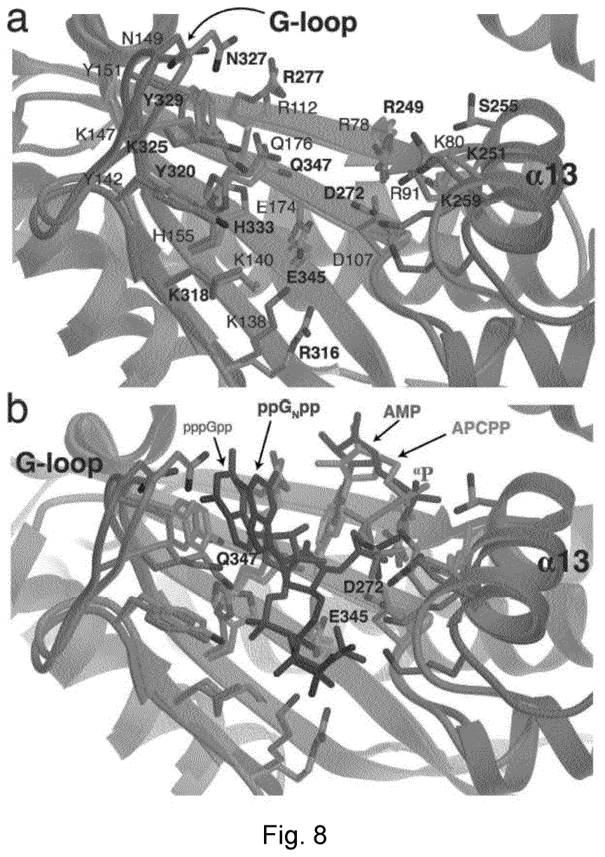

The PC active site of Rel_{Tt}^{NTD} resembles the pre-catalytic state observed in the structure of the RelP SAS enzyme from S. aureus, a single-domain (p)ppGpp synthetase-only RSH enzyme that lacks additional catalytic or regulatory domains. The overall interactions of ppGp_{N}p with the synthetase active site are similar to those observed in the RelP-GTP-APCPP (PDBID 6EWZ) and RelP-pppGpp (PDBID 6EX0) complexes (FIG. 2e, FIG. 8a-b) (Manav et al., Structural basis for (p)ppGpp synthesis by the Staphylococcus aureus small alarmone synthetase RelP, The journal of biological Chemistry, 2018). Only small differences in the orientation of the G-loop16 and the accommodation of the ligand are observed due to the lack of the additional phosphate group that is present in both RelP complexes (FIG. 8b). The coordination of the adenosine group of AMP in the active site also resembles that of the pre-catalytic RelP with the adenosine base stacked between R249 and R277 and the a-phosphate of AMP coordinated in the same manner as that observed in the RelP-GTP-APCPP complex by R249 and K215 (FIG. 8b). In addition, the β1-α13 loop and α13 contribute a patch of positive residues that stabilize the pyrophosphate group transferred to ppGpNp, which is around 2.0 Å away from the site it would occupy in a pre-catalytic state, similar to that of RelP, as part of APCPP. These two active site elements are observed in a similar orientation to that of the β1-α2 loop and α2 of RelP, coordinating the tri-phosphate groups of APCPP (FIG. 8b). Indeed mutations that destabilize ATP binding and affect the interaction of GDP/GTP with the G-loop (R249A, D272, R277A and Y329A) have a strong impact on the activity of the native full length Rel_{Tt} when it is assayed by itself or when the enzyme is activated by either T. thermophilus 70S ribosome initiation complex (70S IC) or the 70S IC with deacylated tRNA^{Val} in the A-site, the ultimate natural activator of ppGpp synthesis by Rel_{Tt} (FIG. 2i).

3. Investigation of Structure-Function Relationship by smFRET.

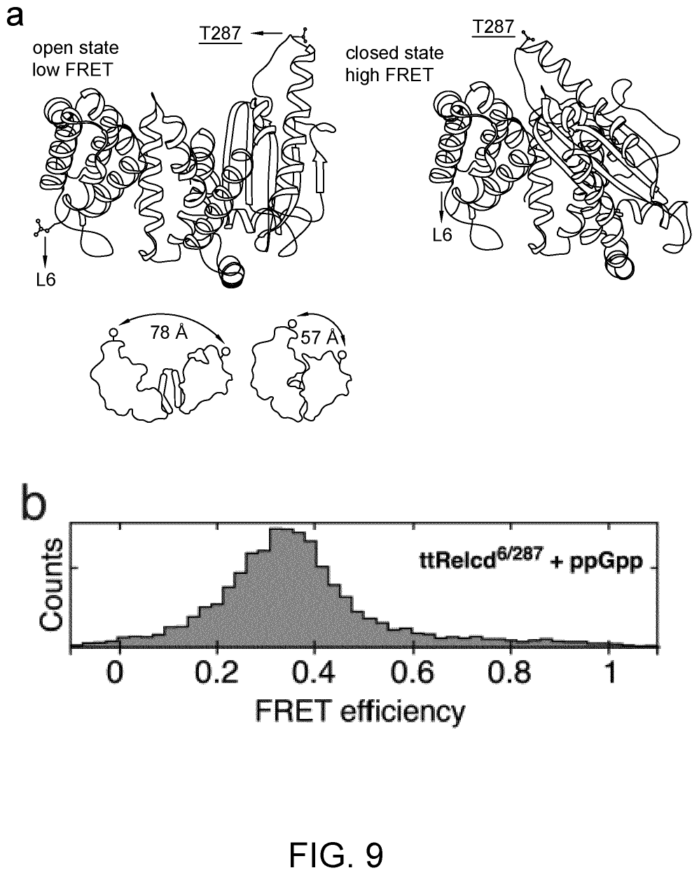

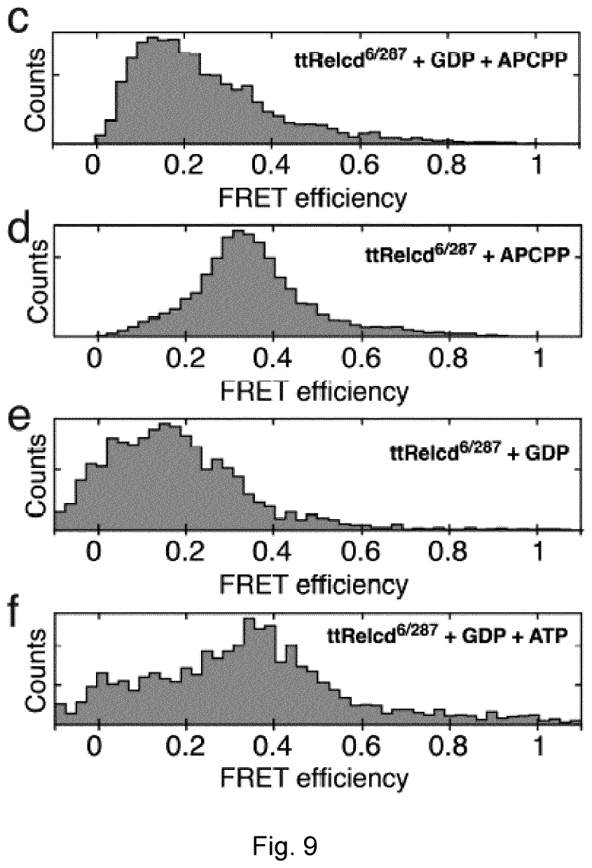

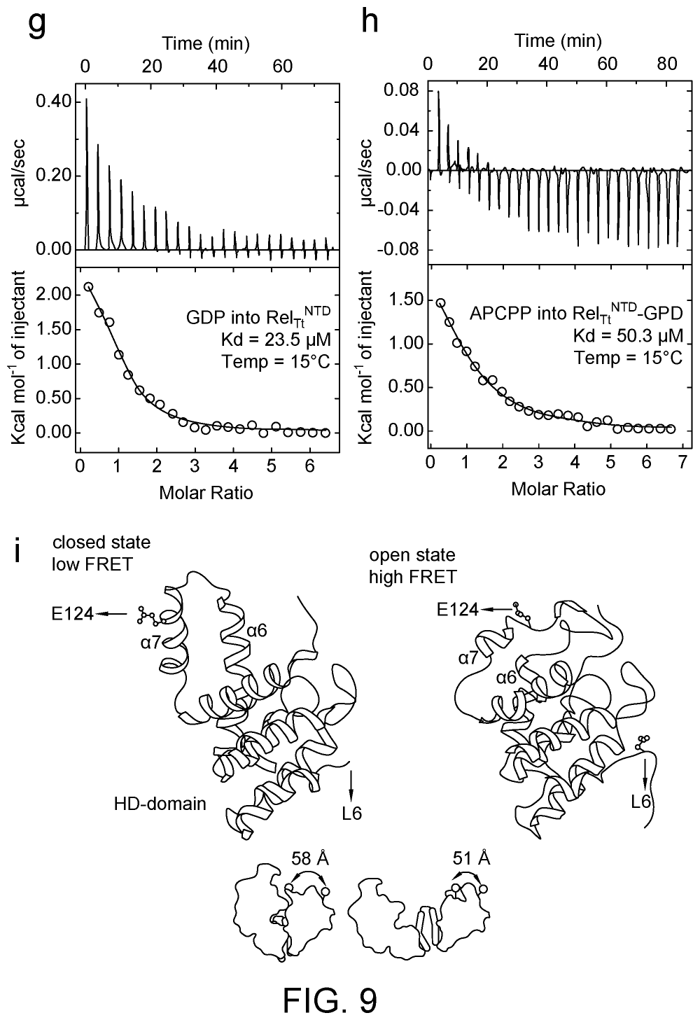

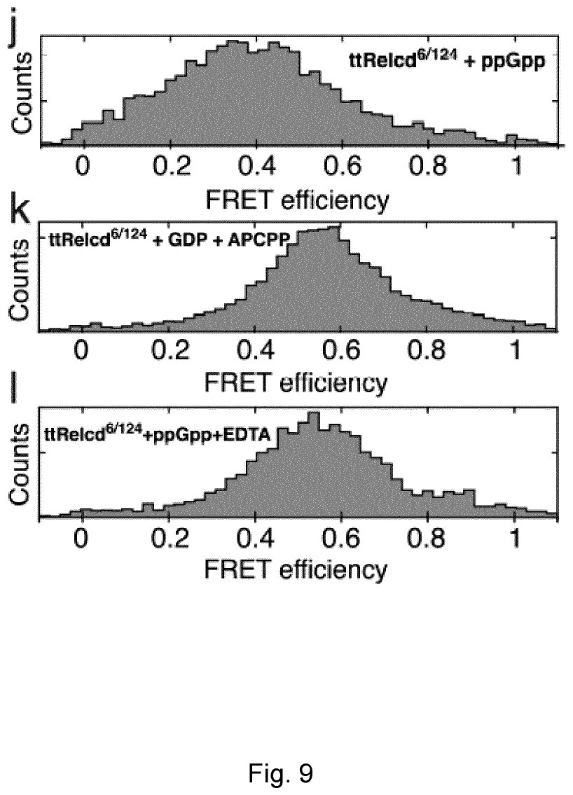

Our structural data suggest that the presence of nucleotides in either the hydrolase or the synthetase domain would prime the enzyme for that particular function, switching off the other catalytic site. Such an allosteric effect would manifest itself in the form of changes in the width of the conformational landscape that would tilt the dynamic equilibrium of the population ensemble towards the favoured state as a function of the concentration of the nucleotides in solution. We directly challenge this hypothesis using single-molecule fluorescence resonance energy transfer (smFRET). For this, we constructed Rel_{Tt}^{NTD}_{6/287}, a variant of Rel_{Tt}^{NTD} that allows fluorescent labels to be attached at cysteine residues introduced at positions 6 and 287 (FIG. 9a). The FRET-averaged inter-dye distance [symbol] R_{DA} [symbol] _{E} predicted for Rel_{Tt}^{NTD}_{6/287} based on our crystal structures is 75 Å for the open form (Rel_{Tt}^{NTD}-AMP-ppGp_{N}p complex) and 57 Å for the closed form (Rel_{Tt}^{NTD}-ppGp_{N}p complex).

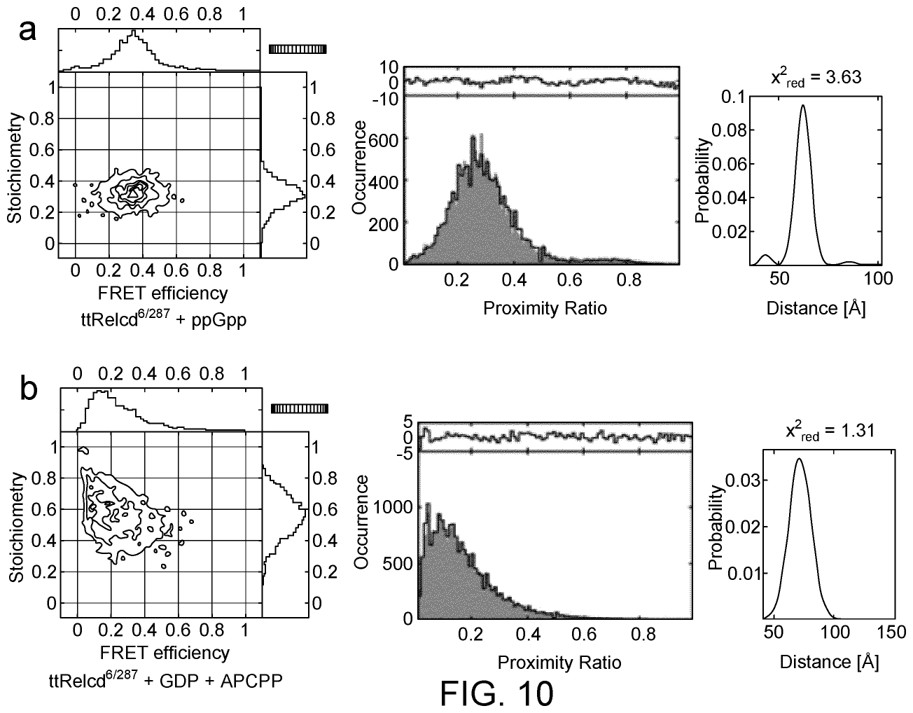

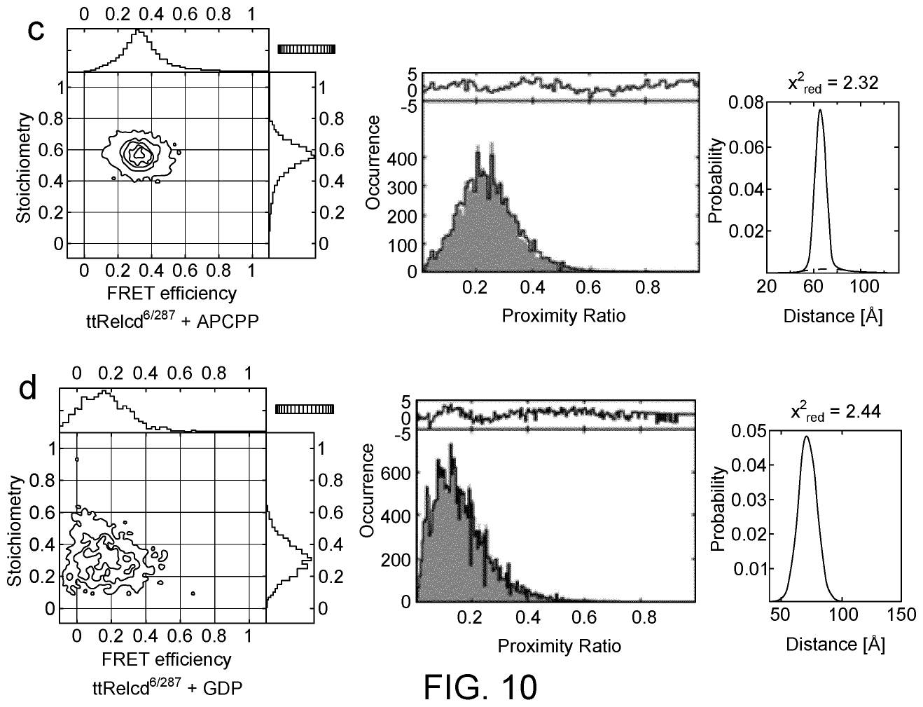

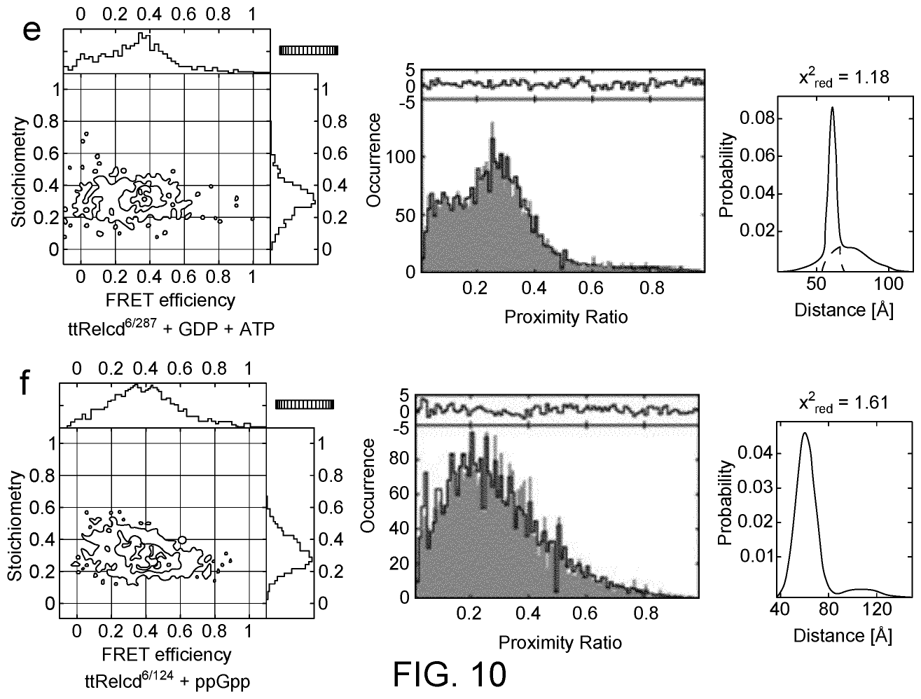

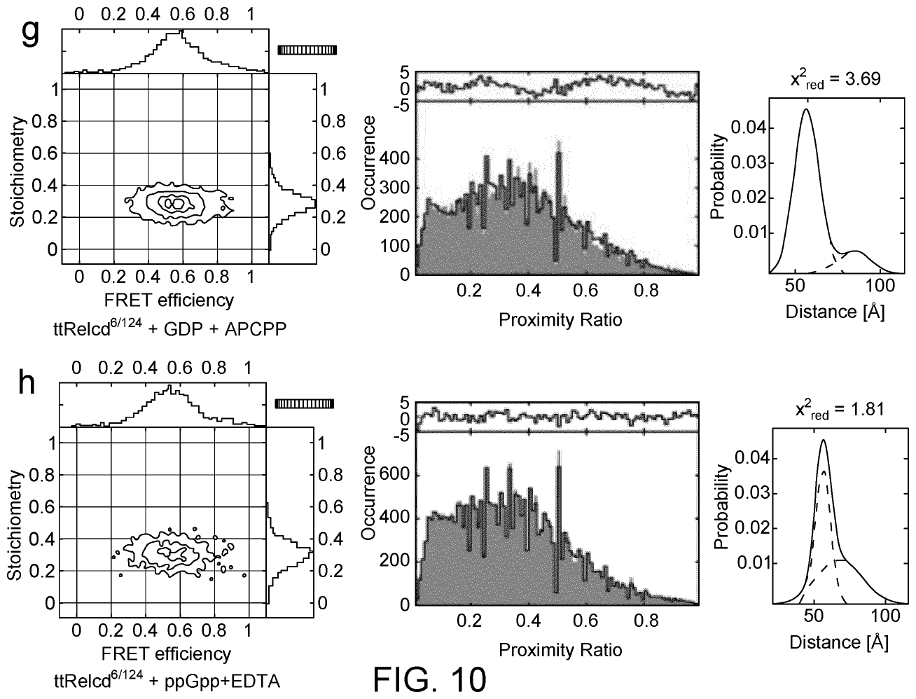

While in the presence of ppGpp, Rel_{Tt}^{NTD}_{6/287} shows a homogenous population with [symbol] R_{DA} [symbol] _{E} of 64 Å (FIG. 9b and FIG. 10a), when incubated with GDP combined with the non-hydrolysable ATP analogue—APCPP—the Rel_{Tt}^{NTD}_{6/287} population shifted to a [symbol] R_{DA} [symbol] _{E} of 72 Å consistent with the opening of the enzyme (FIG. 9c and FIG. 10b). These clearly different conformational states matched the theoretical states expected from the aforementioned crystal structures (FIG. 2b and FIG. 2e). In the presence of APCPP alone, Rel_{Tt}^{NTD}_{6/287} remained in the closed conformation (FIG. 9d and FIG. 10c). Conversely, GDP triggered the opening of the enzyme ( [symbol] R_{DA} [symbol] _{E} of 70 Å) (FIG. 9e and FIG. 10d). Interestingly, when ATP was added after pre-incubating the enzyme with GDP, the ppGpp produced as a result of the reaction returned the equilibrium to the closed state (FIG. 9f and FIG. 10e). This suggests the reaction occurs in a sequential manner with the guanosine substrates binding first in the active site.

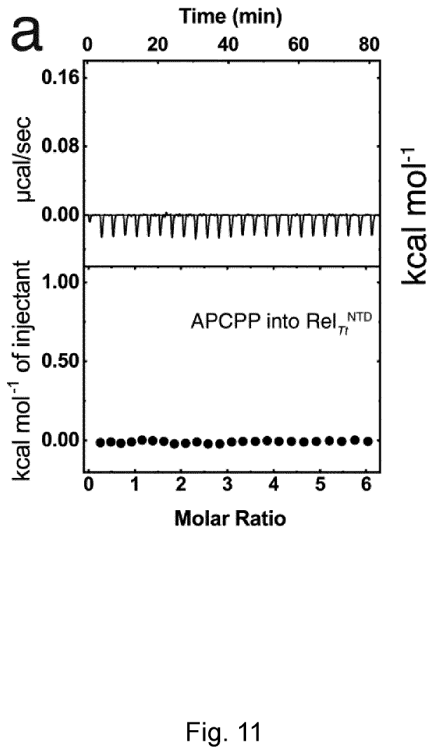

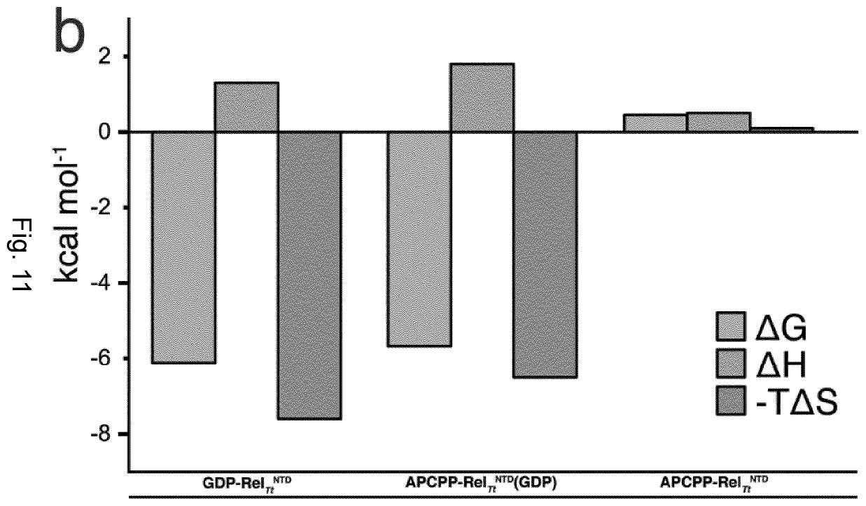

To directly address the sequential binding hypothesis, we monitored the binding of these nucleotides using Isothermal Titration Calorimetry (ITC). GDP binds Rel_{Tt}^{NTD} with an affinity of 23 μM (FIG. 9g and Table 6) and in excellent agreement with the smFRET data APCPP alone does not bind Rel_{Tt}^{NTD} (FIG. 11a and Table 6). However, once the Rel_{Tt}^{NTD}-GDP complex is formed, APCPP binds with an affinity of around 50 μM (FIG. 9h and Table 6). The incorporation of both GDP and APCPP is entropically driven (FIG. 11b and Table 6). In the case of the binding of GDP to the SYN-domain, the release of ordered water molecules from the GDP binding cleft is accompanied by the movement of α6-α7 that partially occludes the HD active center and an increase in the enzyme flexibility as observed in the crystal structure. All these structural changes are consistent with entropically driven binding events. The smFRET data shows that GDP binding is also coupled to the opening of the enzyme which reveals the otherwise buried ATP biding site. In addition, it creates an ‘entropy reservoir’ that drives the binding of APCPP with the release of water molecules from the now exposed binding cleft. This supports the notion that GDP must ‘open’ the enzyme to reveal the ATP binding site, as predicted from the analysis of the crystallographic data.

TABLE 6
Binding parameters obtained from the ITC titrations.

| | | ΔG | ΔH | −TΔS | |
|---|---|---|---|---|---|
| | Kd | (kcal | (kcal | (kcal | |
| Titration | (μM) | mol⁻¹) | mol⁻¹) | mol⁻¹) | n |
| GDP into Rel_{Tt}^{NTD} | 23.0 ± 0.5 | −6.12 ± 0.01 | 1.3 ± 1 | −7.6 ± 1 | 3 |
| GDP into Rel_{Tt}^{NTD}- | 24.7 ± 0.9 | −6.1 ± 0.1 | 0.4 ± 1 | −6.5 ± 1 | 3 |
| APCPP complex | | | | | |
| APCPP into | — | — | — | — | 2 |
| ttRelcd | | | | | |
| APCPP into | 50.4 | −5.67 ± 0.07 | 1.8 ± 1 | −7.5 ± 1 | 3 |
| Rel_{Tt}^{NTD}-GDP | | | | | |
| complex | | | | | |

The binding affinities were determined from fitting a single interaction model to the experimental ITC data according to the experimental setup.
Data represent mean values ± s.d.

We hypothesized that the motions of the α6-α7 loop were coupled to the allosteric switching of the enzyme, constituting a crucial element of the intramolecular crosstalk between domains. In the hydrolase-ON (Rel_{Tt}^{NTD}-ppGpp complex, close state), this loop was projected away from the hydrolase active site allowing the binding of ppGpp whereas in the synthetase-ON state (Rel_{Tt}^{NTD} post-catalytic open state) α6-α7 moved towards the hydrolase active site precluding the binding of ppGpp, effectively switching off the hydrolase function. We used Rel_{Tt}^{NTD}_{6/124}, a Rel_{Tt}^{NTD} variant fluorescently labeled via cysteine residues introduced at residue positions 6 and 124 (FIG. 9i). In the presence of ppGpp, Rel_{Tt}^{NTD}_{6/124} is observed in a low FRET state of [symbol] R_{DA} [symbol] _{E} 62 Å, indicating displacement of the loop away from the active site (FIG. 9j and FIG. 10f). Conversely, when bound to GDP and APCPP, the enzyme switched to a high FRET state ( [symbol] R_{DA} [symbol] _{E} of 55 Å) indicative of the loop movement towards the active site (FIG. 9k and FIG. 10g). These smFRET data are in good agreement with [symbol] R_{DA} [symbol] _{E} estimates based on the structural data that predicts a distance between dyes of 51 Å for the open state and 60 Å for the closed. The removal of the Mn²⁺ ion from the hydrolase site by incubation with EDTA precluded the close conformation even in the presence of ppGpp (FIG. 9l and FIG. 10h). These observations support the role of α6-α7 as an allosteric ‘snaplock’. When the snaplock is stabilized away from the hydrolase active site, the enzyme is predominantly in the closed state and in the hydrolase-ON conformation. By contrast, releasing the snaplock leads to closure of the hydrolase active site and the ‘snap’-opening of the enzyme exposing the synthetase active site (FIG. 7b-c).

4. Biological Significance of the Stretch/Recoil Mechanism.

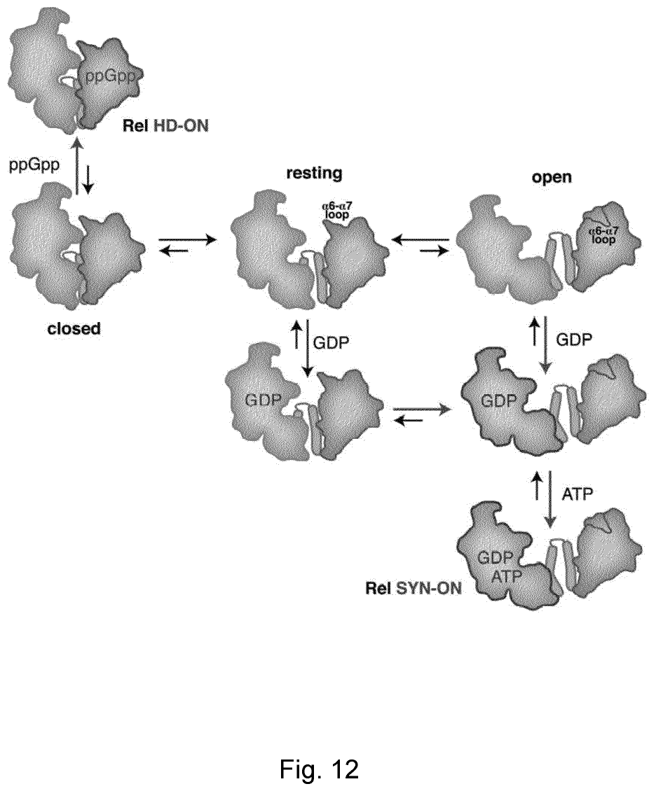

The dynamic modulation of the cellular alarmone levels is paramount to maintenance of cellular homeostasis. Here we showed that Rel enzymes possessing active hydrolase and synthetase domains rely on additional levels of allosteric regulation besides the control that the C-terminal regulatory domains exert, which prevent the occurrence of futile catalytic cycles. The activation of one of the catalytic domains entails the physical blockade and active site misalignment of the other. Our results support the view that the allosteric motion of α-helices α6 and α7 is coupled to the catalytic cycle precluding or allowing the access of substrate to the hydrolase active site whereas the relative conformational state between domains regulates the synthetase function, hindering access to the synthetase site in the close state. This allosteric control provides a bona fide on/off switch that renders one domain completely blocked while the other is active (FIG. 12). In addition, our results suggest that the allosteric control of long RSH enzymes may differ from that of SASs such RelP or RelQ that also bind their substrates in an ordered sequence but incorporate first with ATP and then GDP or GTP (Steinchen et al., Catalytic mechanism and allosteric regulation of an oligomeric (p)ppGpp synthetase by an alarmone, PNAS USA, 2015). We show that in long RSH enzymes this sequence is reversed. This divergence in the catalytic mechanism is most likely due to the loss of the hydrolase domain and with it the requirement of triggering an open state in SASs to bind ATP. The current view of the role of nucleotides in the control of Rel enzymes, based on known structures, does not provide a conclusive explanation as to how the conformational interplay between the two catalytic domains is linked to the activation of one site to the detriment of the other. Moreover in all known structures of different RSH enzymes the two catalytic domains are observed in similar conformations that are incompatible with an active synthetase state (Hogg et al., Cell, 2004, Singal et al., FEBS letters, 2017, Arenz et al., Nucleic acids research, 2016, Brown et al., Nature 2016, and Loveland et al., Elife, 2016).

The regulation of catalysis of bifunctional enzymes is usually dominated by allosteric transitions preventing the occurrence of futile cycles (Okar et al., PFK-2/FBPase-2: maker and breaker of the essential biofactor fructose-2,6-bisphosphate, Trends in biochemical sciences, 2001). The stretch/recoil mechanism we propose provides the basis for the macromolecular control of the enzyme by its opposing substrates. The synchronous action of this additional regulatory layer, together with the spatial control of the ribosome as the docking platform of the enzyme for (p)ppGpp synthesis (Arenz et al., Nucleic acids research, 2016, Brown et al., Nature 2016, and Loveland et al., Elife, 2016) or the PTS for ppGpp hydrolysis (Ronneau et al., Regulation of (p)ppGpp hydrolysis by a conserved archetypal regulatory domain, Nucleic acids research, 2019), denote a tightly regulated mechanism that prevents the occurrence of futile catalytic cycles and facilitates the action of the alarmone as a bacterial phenotypic switch.

5. Discovery of Novel ppGpp Allosteric Sites.

The alarmone nucleotides guanosine pentaphosphate (pppGpp) and tetraphosphate (ppGpp)—collectively referred to as (p)ppGpp—are central regulators of bacterial metabolism and stress responses, with effects on antibiotic tolerance and virulence (Gaca et al., Many means to a common end: the intricacies of (p)ppGpp metabolism and its control of bacterial homeostasis, J Bacteriology, 2015; Hauryliuk et al., Recent functional insights into the role of (p)ppGpp in bacterial physiology, Nat Rev Microbiol, 2015; Liu et al., Diversity in (p)ppGpp metabolism and effectors, Curr Opin Microbiol, 2015). The concentration of (p)ppGpp in the cell is controlled by enzymes belonging to the RelA/SpoT Homologue (RSH) protein family (Atkinson et al., The RelA/SpoT homolog (RSH) superfamily: distribution and functional evolution of ppGpp synthetases and hydrolases across the tree of life, PLoS One, 2011). RSH family members synthesize (p)ppGpp by transferring the pyrophosphate group of ATP onto either GTP or GDP, and/or degrade it by removing the diphosphate and converting the alarmone back to GTP or GDP. The pentaphosphate alarmone pppGpp is a much more potent activator than the tetraphosphate ppGpp (Kudrin et al., The ribosomal A-site finger is crucial for binding and activation of the stringent factor RelA, Nucleic Acids Res, 2018). However, the molecular details of this allosteric regulatory mechanism and the location of RelA's (p)ppGpp binding site have remained elusive due to the challenging nature of RelA (low solubility and stability) combined with its considerable structural complexity. Long RSHs are comprised of an N-terminal enzymatic half (N terminal domain region, NTD) and C-terminal regulatory half (C terminal domain region, CTD). The NTD contains two domains: the (p)ppGpp hydrolysis (HD) and (p)ppGpp synthesis (SYNTH) domains, while the CTD is comprised of the TGS (ThrRS, GTPase and SpoT), Helical, ZFD (Zinc Finger Domain; equivalent to CC, conserved cysteine as per (Atkinson et al., The RelA/SpoT homolog (RSH) superfamily: distribution and functional evolution of ppGpp synthetases and hydrolases across the tree of life, PLoS One, 2011)) and RRM (RNA recognition motif; equivalent to ACT, aspartokinase, chorismate mutase and TyrA, as per (Atkinson et al., The RelA/SpoT homolog (RSH) superfamily: distribution and functional evolution of ppGpp synthetases and hydrolases across the tree of life, PLoS One, 2011)).

Investigations of E. coli RelA (Agirrezabala et al., The ribosome triggers the stringent response by RelA via a highly distorted tRNA. EMBO Rep, 2013; Arenz et al., The stringent factor RelA adopts an open conformation on the ribosome to stimulate ppGpp synthesis. Nucleic Acids Res, 2016; Brown et al., Ribosome-dependent activation of stringent control. Nature, 2016; Gropp et al., Regulation of Escherichia coli RelA requires oligomerization of the C-terminal domain. 2001; Loveland et al., Ribosome*RelA structures reveal the mechanism of stringent response activation, Elife, 2016; Turnbull et al., Intramolecular Interactions Dominate the Autoregulation of Escherichia coli Stringent Factor RelA, Frontiers in Microbiology, 2019) as well as Rel enzymes from Bacillus subtilis by us, Mycobacterium tuberculosis (Avarbock et al., Functional regulation of the opposing (p)ppGpp synthetase/hydrolase activities of RelMtb from Mycobacterium tuberculosis, Biochemistry, 2005; Jain et al., Molecular dissection of the mycobacterial stringent response protein Rel, Protein Sci, 2006), Thermus thermophilus (Tamman et al., Nucleotide-mediated allosteric regulation of bifunctional Rel enzymes, BioRxiv, 2019) and Streptococcus equisimilis (Hogg et al., Conformational antagonism between opposing active sites in a bifunctional RelA/SpoT homolog modulates (p)ppGpp metabolism during the stringent response [corrected], Cell, 2004; Mechold et al., Intramolecular regulation of the opposing (p)ppGpp catalytic activities of Rel(Seq), the Rel/Spo enzyme from Streptococcus equisimilis, J Bacteriol, 2002) have been instrumental for our understanding of the molecular regulation of long ribosome-associated RSHs. Collectively these studies have established that i) the CTD mediates contact with the rRNA and a highly distorted A-site tRNA—so-called A/R tRNA—of the ‘starved’ ribosomal complex, ii) the CTD transmits the allosteric signal to regulate the synthetic activity of the NTD and, iii) the synthetic and hydrolytic activities of SYNTH and HD domains of the NTD allosterically oppose each other's enzymatic activities. This network of intramolecular allosteric regulation is likely to be exploited by (p)ppGpp in its positive regulation of RelA. We have identified a “hot spot” using hydrogen-deuterium exchange mass spectrometry (HDX-MS). This site a located in the region that connects the hydrolase and synthetase domains of RelANTD and consists of α-helices α8, α9, α10, α11′ and α12, involving residues K164, D200, Y201, R204, Y211, K212, H219, R221, R222 and R225. Y211 is particularly important for the interaction and substitutions different from F or H are not permissive decreasing significantly the turnover of the enzyme and its affinity for pppGpp.

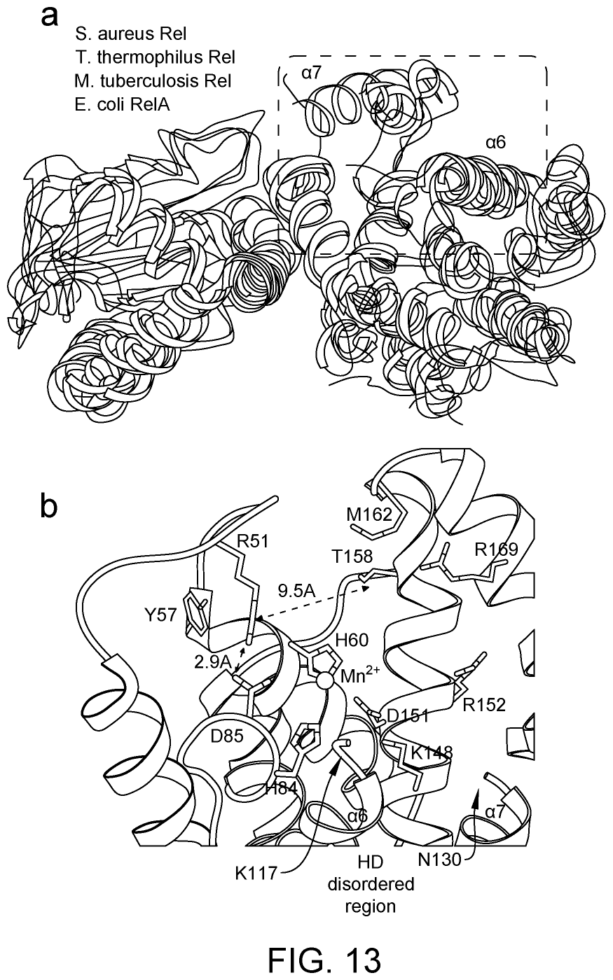

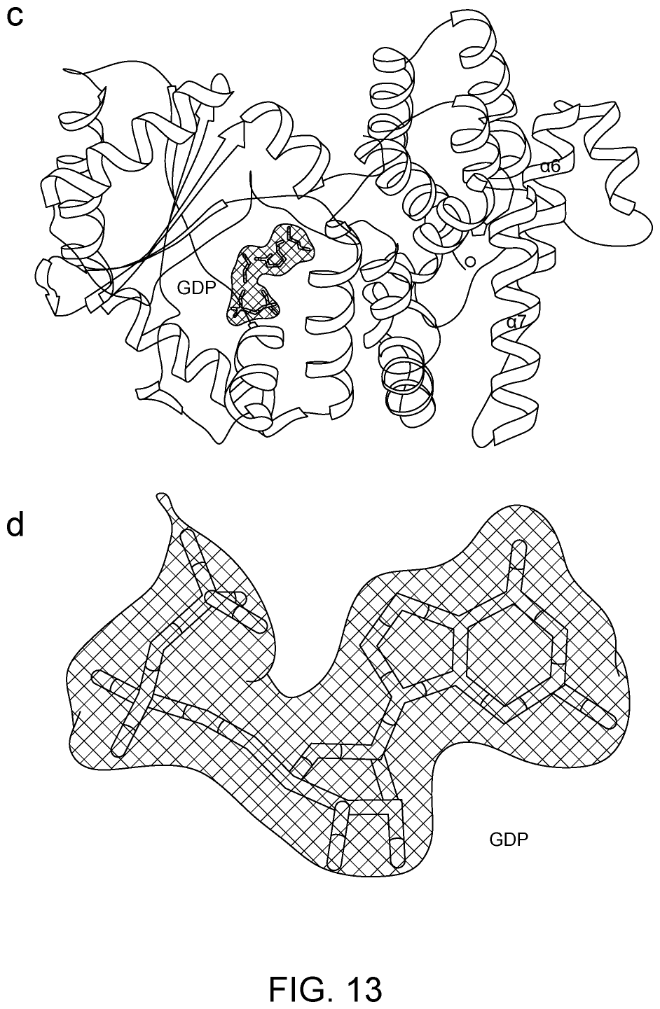

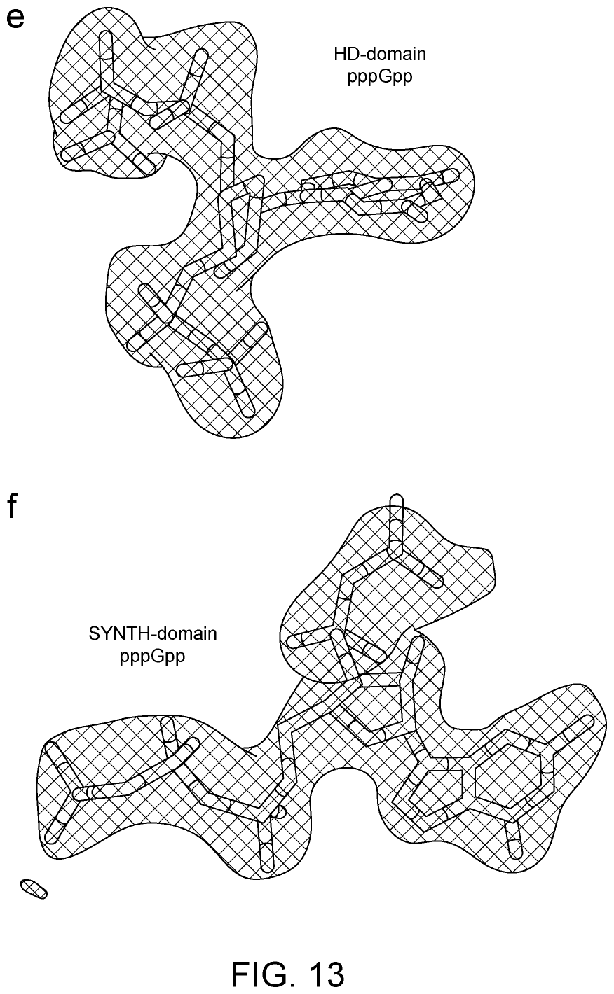

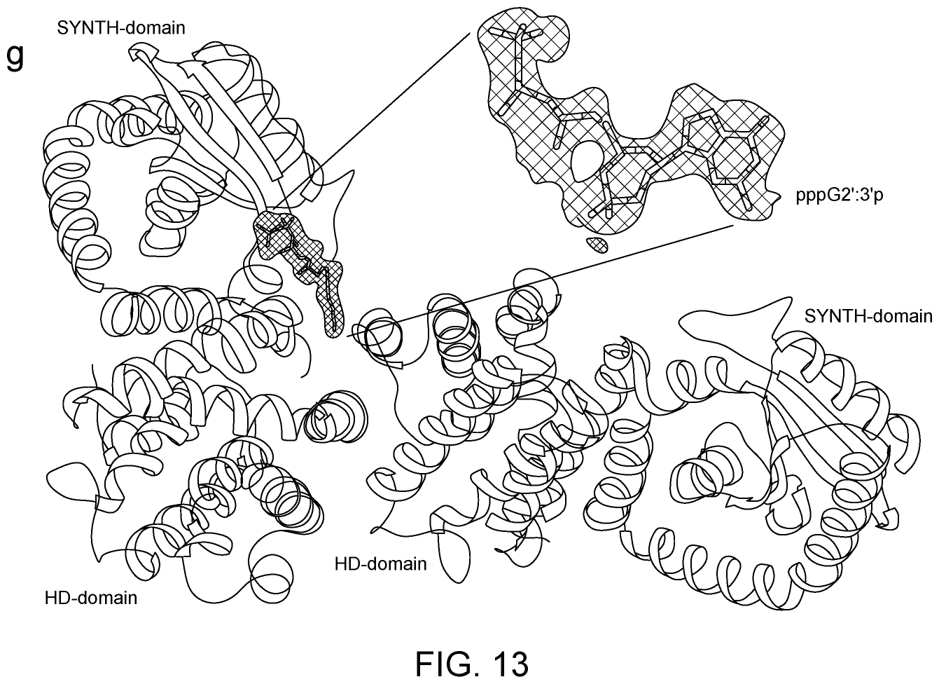

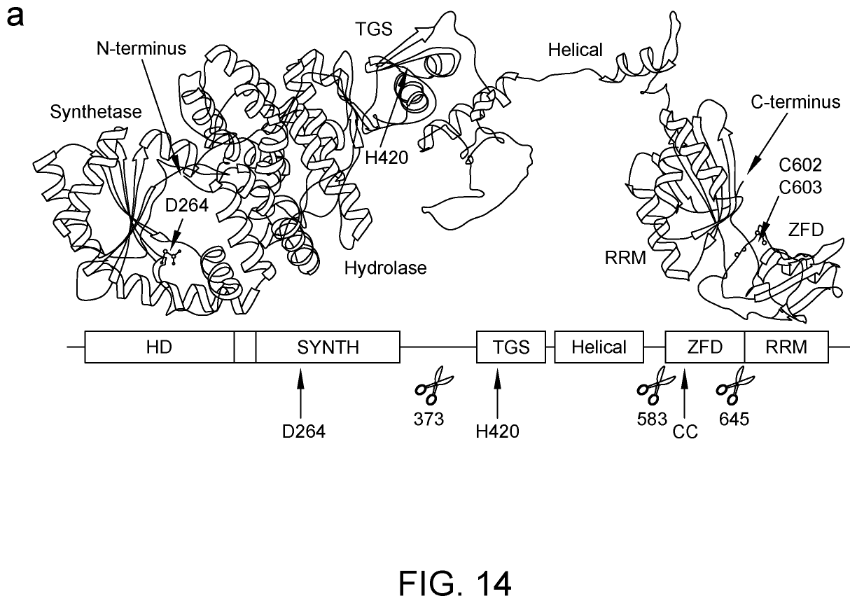

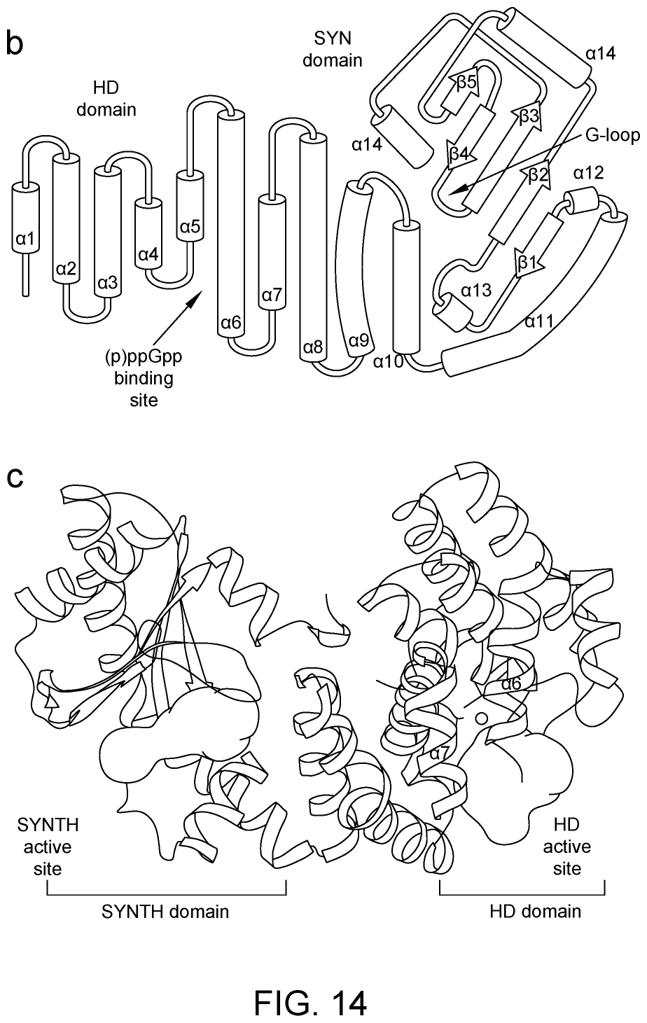

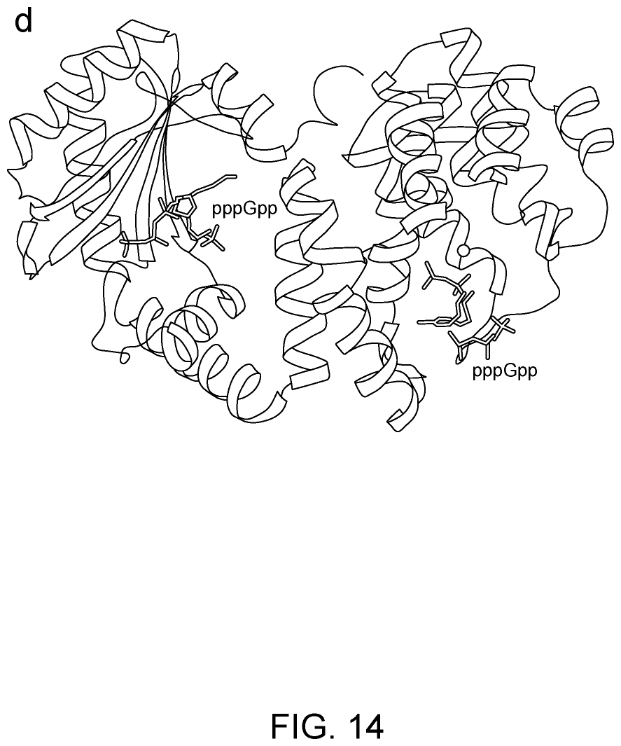

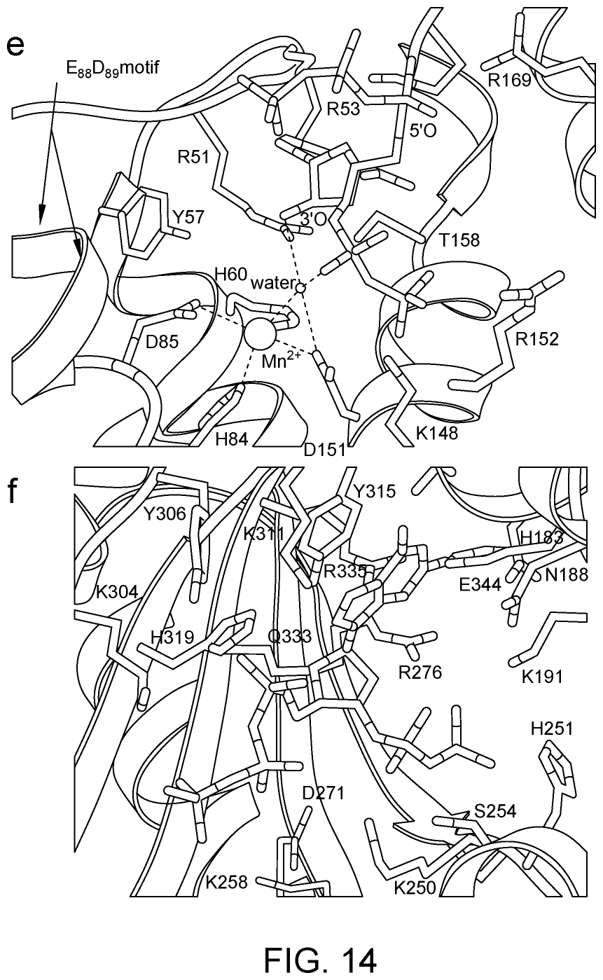

6. Putative Novel Site that Stabilizes the Enzyme in a Resting-Like Inactive State (FIG. 13 and FIG. 14).

The resting apo state of the catalytic NTD region of Rel/RelA enzymes is structurally conserved (FIG. 13A). The NTD is the catalytic core of long RSH enzymes, with its constituent SYNTH and HD domains regulating each other's activities. Therefore, to understand how the enzymatic activities of full-length Rel are regulated by ligands such as starved ribosomal complexes and tRNA, it is essential to understand the internal workings of the NTD itself. We solved the structure of the N-terminal catalytic domain region of Rel (FIG. 13B-C) from the human opportunistic Firmicute pathogen S. aureus (RelSaNTD).

The structure of RelSaNTD in a catalytically resting apo-state resembles that of other nucleotidefree Rel and RelA enzymes, both in terms of the overall fold of its catalytic domains, and their relative conformational state. While in the apo-state the hydrolase (HD) domain coordinates the Mn²⁺ ion that is essential for catalysis, the active site is not properly organized. R51 is misaligned and hydrogen-bonds D85 instead of the conserved T158(9.5 Å in the apo-state). The H-bond to T158 is crucial to orient the guanidine group for coordinating the guanine of (p)ppGpp. In addition this free state includes the increased flexibility of the region involving residues 117-130 of α6-α7 region for which we could not observe any density in the crystal structure. Recent structural studies of T. thermophilus Rel NTD (RelTtNTD) have identified the α6-α7 structural element as an allosteric switch linking the activation of one of the catalytic domains with the inactivation of the other. The aforementioned disorder in in the active site of apo-RelSaNTD, suggests that the apo-state is not compatible with active hydrolysis as observed in the case of apo-RelTtNTD. Therefore substrate binding to the HD site is likely required to trigger a conformational change in Rel that aligns the active site and allows (p)ppGpp hydrolysis. In the other catalytic domain, the dimensions of the SYNTH active site of RelSaNTD (with a volume of around 700 Å3) also resemble more that of RelTtNTD in the SYNTH-OFF conformation (around 780 Å3) with a completely buried ATP binding site and only the GDP binding site being exposed, than that of the active site of RelTtNTD in the SYNTH-ON which is approximately 1800 Å3 (FIG. 14C).

Substrate binding is required but not sufficient to lock a particular active catalytic state to gain insight into the intra-molecular regulation of the Rel catalytic domains by nucleotide substrates, we solved structures of RelSaNTD in a bound to either GDP or pppGpp. The structure of the RelSaNTD:GDP complex is similar to that of GDP-bound S. equisimilis Rel NTD (RelSeqNTD). GDP binding triggers only minor conformational changes compared to the unbound resting enzyme. These changes observed in the RelSaNTD:GDP complex are not sufficient to stabilize a conformation that is fully compatible with the active synthetase state. Such conformation was observed in for T. thermophilus Rel bound to both ppGpp and AMP. In this post-catalytic state, the enzyme was in an open conformation that involved the rotation of the SYNTH domain with respect to the central linker region that connects both domains, which exposed the ATP binding site. The inefficacy of GDP to trigger this open conformation could have regulatory implications in the outcome of catalysis. Thus the preference for GDP or GTP would be decided by the molecule that is more effective in stabilizing the active SYNTH conformation, with the extra phosphate of GTP likely the crucial group to trigger the open state. The structure of T. thermophilus Rel bound with ppGpp bound in the HD active site shows the enzyme undergoes a conformational change that results in a more compact NTD. We solved the structure of RelSaNTD in complex with pppGpp (FIG. 14D). While pppGpp is bound in an orientation strongly reminiscent of that observed in the RelTtNTD:ppGpp complex (FIG. 14E), the molecule is not hydrolysed due to the misalignment of the active site residues. This is likely the result of an additional molecule of pppGpp bound in the SYNTH active site of the enzyme (FIG. 13E-G).

The overall structure of the RelSaNTD:pppGpp complex is very similar to that of the resting state and the RelSaNTD:GDP complex. The presence of pppGpp in the HD active site triggers some notable conformational changes that are however not sufficient to induce a fully active hydrolase state as observed in the RelTtNTD:ppGpp complex. In general, Rel HD active sites are defined by a hydrophobic region that engages the base of the nucleotide and two opposing acid and basic sites that accommodate the 3′ and 5′ groups. In the RelSaNTD:pppGpp complex, the guanosine base of the alarmone is stacked between R51 (that now is within 3.3 Å of T158—compared to 9.5 Å in the apo-state and 9.0 Å in the GDP complex—) and M162 and makes additional Van der Waals interactions with K52, Y57, K59 and N155. As observed in RelSeq, the majority of hydrogen bond interactions of the base with the enzyme are via the backbone amide and carbonyl groups of α-helices α3 and α8. At the 5′ end the tri-phosphate group is exposed to the bulk solvent with only the α-phosphate interacting with R169 (FIG. 14E). The 3′-pyrophosphate moiety of the alarmone is oriented towards the acidic patch of the active site near the Mn2+ ion. This patch contains the D85, the E88D89 motif and D151, all involved in conditioning (p)ppGpp for the nucleophilic attack that triggers hydrolysis and activating of a water molecule for a nucleophilic attack. However the aforementioned misalignment of this patched positions E88D89 motif away from the scissile bond, likely preventing hydrolysis and allowing a rare glimpse of the pppGpp in the HD active site (FIG. 14E). The observed binding mode of pppGpp confirms the role of the Mn2+ ion in hydrolysis. In the structure, the Mn2+ is coordinating a water molecule that is directly poised for a nucleophilic attack on the phospho-esther bond (FIG. 14E). The α6-α7 region has been suggested as a conformational snaplock and specificity determinant of Rel, with a crucial role in the hydrolysis of (p)ppGpp. In the RelSaNTD:pppGpp complex residues from 112 to 130 from α6-α7 are not visible in th electron density suggesting that this partial disorder in the HD site involving both, the acid and basic sections, is strongly coupled a reduced hydrolysis by the enzyme, likely by interfering with the nucleophilic attack on the phosphor-esther bond. In addition to the pppGpp observed in the HD active site, the RelSaNTD:pppGpp structure also contains a pppGpp molecule in the SYNTH active site (FIG. 14D-F and Supplementary Figure Manav et al., Structural basis for (p)ppGpp synthesis by the Staphylococcus aureus small alarmone synthetase RelP, J Biol Chem, 2018). This SYNTH active site conformation is similar to the post-catalytic state observed in the pppGpp-bound complex of the single-domain Small Alarmone Synthetase (SAS) RelP from S. aureus (Manav et al., Structural basis for (p)ppGpp synthesis by the Staphylococcus aureus small alarmone synthetase RelP, J Biol Chem, 2018). Given the antagonistic allosteric coupling between the two catalytic domains in Rel enzymes, the presence of pppGpp in both sites precludes the authentic HDON state attained in the presence of the pppGpp substrate in the HD active site only. This hypothesis is consistent with the disordered acid patch of the HD domain in our RelSaNTD:pppGpp structure which, in turn, allows binding of pppGpp without hydrolysis.

The lattice constrains and SYNTH-domain occupancy, combined with the observed HD active site disorder, likely resulted in a slow rate of hydrolysis that allowed us to capture a leaving product of the hydrolase reaction at the interface the between the two molecules of the asymmetric unit.

Interestingly the reaction product is the unusual GTP derivative, guanosine 5′-triphosphate-2′:3′-cyclic mono-phosphate (pppG2′:3′p) (FIG. 13G). This observation is a direct recall to the RelSeqppG2′:3′p complex in which a partially hydrolyzed cyclic molecule (5′-diphosphate-2′:3′-cyclic mono-phosphate) is observed bound in the RelSeq active site. The biological relevance of this molecule remains a question, however such side product of hydrolysis can also be observed in solution.

Taken together, our structural results underscore the complexity of the activation of the catalytic domains of Rel enzymes. While substrate binding has been shown to be crucial for priming the enzyme for a particular catalytic function (Tamman et al., Nucleotide-mediated allosteric regulation of bifunctional Rel enzymes, BioRxiv, 2019; Hogg et al., Conformational antagonism between opposing active sites in a bifunctional RelA/SpoT homolog modulates (p)ppGpp metabolism during the stringent response [corrected]. Cell, 2004), this event on its own is not sufficient to guarantee the complete stabilization of an active catalytic state. This leaves the enzyme vulnerable to inhibition due to the serendipitous binding of nucleotides in both catalytic domains. In RelSeq, the simultaneous occupancy of both active sites precludes the enzyme from adopting a fully active catalytic conformation. This seems to be the case for the RelSaNTD:pppGpp as well. Considering the high efficiency of the enzyme in vivo, it is likely that additional layers of regulation are in place to fully stabilize of one conformation depending on a particular cellular state and prevent the occupancy of both sites at the same time.

TABLE 1
Rel unbound form crystal structure atomic coordinates: This is an intermediate
conformation (inactive), different from the open and not closed.

| | | | | | | | | | | |
|---|---|---|---|---|---|---|---|---|---|---|
| HELIX | 1 | 1 | TRP | C | 9 | LEU | C | 12 | 1 | 4 |
| HELIX | 2 | 2 | PRO | C | 21 | ALA | C | 37 | 1 | 17 |
| HELIX | 3 | 3 | THR | C | 51 | GLY | C | 62 | 1 | 12 |
| HELIX | 4 | 4 | ALA | C | 67 | LEU | C | 75 | 1 | 9 |
| HELIX | 5 | 5 | PRO | C | 86 | PHE | C | 93 | 1 | 8 |
| HELIX | 6 | 6 | PRO | C | 95 | VAL | C | 107 | 1 | 13 |
| HELIX | 7 | 7 | GLN | C | 128 | MET | C | 133 | 1 | 6 |
| HELIX | 8 | 8 | VAL | C | 137 | ARG | C | 152 | 1 | 16 |
| HELIX | 9 | 9 | PRO | C | 159 | ARG | C | 179 | 1 | 21 |
| HELIX | 10 | 10 | GLY | C | 183 | LEU | C | 197 | 1 | 15 |
| HELIX | 11 | 11 | PRO | C | 199 | ALA | C | 211 | 1 | 13 |
| HELIX | 12 | 12 | GLN | C | 213 | ARG | C | 233 | 1 | 21 |
| HELIX | 13 | 13 | SER | C | 254 | MET | C | 259 | 1 | 6 |
| HELIX | 14 | 14 | LEU | C | 293 | LEU | C | 309 | 1 | 17 |

| | | | | | | | | | | |
|---|---|---|---|---|---|---|---|---|---|---|
| SHEET | 1 | 1 | 1 | ALA | C | 275 | ILE | C | 279 | 0 |
| SHEET | 2 | 2 | 1 | VAL | C | 317 | ASP | C | 319 | 0 |
| SHEET | 3 | 3 | 1 | LEU | C | 332 | ALA | C | 338 | 0 |
| SHEET | 4 | 4 | 1 | LEU | C | 342 | ARG | C | 349 | 0 |

| | | | | | | | | | | | | |
|---|---|---|---|---|---|---|---|---|---|---|---|---|
| ATOM | 1 | N | ALA | C | 0 | 86.594 | 39.725 | 5.089 | 1.00 | 102.74 | | N |
| ANISOU | 1 | N | ALA | C | 0 | 12608 | 9713 | 16713 | −6606 | −2687 | −343 | N |
| ATOM | 2 | CA | ALA | C | 0 | 86.876 | 38.305 | 5.271 | 1.00 | 98.82 | | C |
| ANISOU | 2 | CA | ALA | C | 0 | 11865 | 9713 | 15970 | −6421 | −2528 | −286 | C |
| ATOM | 3 | C | ALA | C | 0 | 87.287 | 37.606 | 3.951 | 1.00 | 100.17 | | C |
| ANISOU | 3 | C | ALA | C | 0 | 11935 | 10036 | 16091 | −6484 | −2251 | 92 | C |
| ATOM | 4 | O | ALA | C | 0 | 86.625 | 37.773 | 2.919 | 1.00 | 98.64 | | O |
| ANISOU | 4 | O | ALA | C | 0 | 11950 | 9636 | 15895 | −6434 | −2133 | 297 | O |
| ATOM | 5 | CB | ALA | C | 0 | 85.682 | 37.610 | 5.910 | 1.00 | 96.27 | | C |
| ANISOU | 5 | CB | ALA | C | 0 | 11666 | 9517 | 15395 | −6022 | −2548 | −459 | C |
| ATOM | 6 | N | ALA | C | 1 | 88.408 | 36.844 | 4.006 | 1.00 | 95.94 | | N |
| ANISOU | 6 | N | ALA | C | 1 | 11064 | 9876 | 15511 | −6585 | −2163 | 165 | N |
| ATOM | 7 | CA | ALA | C | 1 | 89.030 | 36.075 | 2.914 | 1.00 | 94.34 | | C |
| ANISOU | 7 | CA | ALA | C | 1 | 10678 | 9919 | 15247 | −6642 | −1916 | 468 | C |
| ATOM | 8 | C | ALA | C | 1 | 89.236 | 34.596 | 3.337 | 1.00 | 94.08 | | C |
| ANISOU | 8 | C | ALA | C | 1 | 10417 | 10350 | 14980 | −6383 | −1847 | 431 | C |
| ATOM | 9 | O | ALA | C | 1 | 88.801 | 34.224 | 4.435 | 1.00 | 93.02 | | O |
| ANISOU | 9 | O | ALA | C | 1 | 10295 | 10319 | 14728 | −6185 | −1987 | 196 | O |
| ATOM | 10 | CB | ALA | C | 1 | 90.361 | 36.710 | 2.547 | 1.00 | 99.04 | | C |
| ANISOU | 10 | CB | ALA | C | 1 | 11062 | 10515 | 16052 | −7067 | −1901 | 578 | C |
| ATOM | 11 | N | VAL | C | 2 | 89.889 | 33.752 | 2.492 | 1.00 | 88.04 | | N |
| ANISOU | 11 | N | VAL | C | 2 | 9451 | 9864 | 14137 | −6378 | −1645 | 654 | N |
| ATOM | 12 | CA | VAL | C | 2 | 90.074 | 32.323 | 2.829 | 1.00 | 84.97 | | C |
| ANISOU | 12 | CA | VAL | C | 2 | 8881 | 9873 | 13532 | −6110 | −1598 | 629 | C |
| ATOM | 13 | C | VAL | C | 2 | 91.461 | 32.061 | 3.570 | 1.00 | 90.37 | | C |
| ANISOU | 13 | C | VAL | C | 2 | 9201 | 10882 | 14254 | −6234 | −1702 | 497 | C |
| ATOM | 14 | O | VAL | C | 2 | 92.223 | 32.990 | 3.887 | 1.00 | 91.95 | | O |
| ANISOU | 14 | O | VAL | C | 2 | 9283 | 11009 | 14646 | −6536 | −1812 | 405 | O |
| ATOM | 15 | CB | VAL | C | 2 | 89.811 | 31.370 | 1.591 | 1.00 | 85.74 | | C |
| ANISOU | 15 | CB | VAL | C | 2 | 8976 | 10113 | 13489 | −5944 | −1352 | 888 | C |
| ATOM | 16 | CGI | VAL | C | 2 | 89.767 | 29.890 | 1.982 | 1.00 | 82.48 | | C |
| ANISOU | 16 | CGI | VAL | C | 2 | 8461 | 10028 | 12851 | −5627 | −1333 | 849 | C |
| ATOM | 17 | CG2 | VAL | C | 2 | 88.502 | 31.722 | 0.893 | 1.00 | 83.71 | | C |
| ANISOU | 17 | CG2 | VAL | C | 2 | 9056 | 9549 | 13201 | −5837 | −1269 | 996 | C |
| ATOM | 18 | N | GLY | C | 3 | 91.659 | 30.793 | 3.939 | 1.00 | 85.02 | | N |
| ANISOU | 18 | N | GLY | C | 3 | 8384 | 10537 | 13385 | −5973 | −1696 | 465 | N |
| ATOM | 19 | CA | GLY | C | 3 | 92.839 | 30.249 | 4.581 | 1.00 | 85.73 | | C |
| ANISOU | 19 | CA | GLY | C | 3 | 8142 | 10983 | 13448 | −5978 | −1788 | 353 | C |
| ATOM | 20 | C | GLY | C | 3 | 93.628 | 29.439 | 3.578 | 1.00 | 88.63 | | C |
| ANISOU | 20 | C | GLY | C | 3 | 8262 | 11641 | 13772 | −5951 | −1607 | 540 | C |
| ATOM | 21 | O | GLY | C | 3 | 94.563 | 29.974 | 2.970 | 1.00 | 91.78 | | O |
| ANISOU | 21 | O | GLY | C | 3 | 8439 | 12117 | 14317 | −6239 | −1534 | 615 | O |
| ATOM | 22 | N | ALA | C | 4 | 93.218 | 28.169 | 3.340 | 1.00 | 80.46 | | N |
| ANISOU | 22 | N | ALA | C | 4 | 7276 | 10763 | 12534 | −5618 | −1527 | 619 | N |
| ATOM | 23 | CA | ALA | C | 4 | 93.938 | 27.279 | 2.424 | 1.00 | 80.41 | | C |
| ANISOU | 23 | CA | ALA | C | 4 | 7033 | 11053 | 12466 | −5533 | −1372 | 761 | C |
| ATOM | 24 | C | ALA | C | 4 | 93.246 | 26.966 | 1.093 | 1.00 | 85.19 | | C |
| ANISOU | 24 | C | ALA | C | 4 | 7797 | 11560 | 13011 | −5443 | −1143 | 991 | C |
| ATOM | 25 | O | ALA | C | 4 | 92.106 | 26.493 | 1.081 | 1.00 | 82.21 | | O |
| ANISOU | 25 | O | ALA | C | 4 | 7703 | 11022 | 12511 | −5218 | −1120 | 1024 | O |
| ATOM | 26 | CB | ALA | C | 4 | 94.283 | 25.996 | 3.127 | 1.00 | 80.05 | | C |
| ANISOU | 26 | CB | ALA | C | 4 | 6861 | 11306 | 12249 | −5223 | −1480 | 661 | C |
| ATOM | 27 | N | ASP | C | 5 | 93.973 | 27.186 | −0.032 | 1.00 | 85.25 | | N |
| ANISOU | 27 | N | ASP | C | 5 | 7597 | 11704 | 13089 | −5623 | −972 | 1144 | N |
| ATOM | 28 | CA | ASP | C | 5 | 93.500 | 26.942 | −1.408 | 1.00 | 84.21 | | C |
| ANISOU | 28 | CA | ASP | C | 5 | 7562 | 11536 | 12896 | −5565 | −745 | 1367 | C |
| ATOM | 29 | C | ASP | C | 5 | 93.749 | 25.484 | −1.803 | 1.00 | 88.11 | | C |
| ANISOU | 29 | C | ASP | C | 5 | 7910 | 12352 | 13216 | −5237 | −677 | 1384 | C |
| ATOM | 30 | O | ASP | C | 5 | 94.816 | 25.114 | −2.309 | 1.00 | 88.76 | | O |
| ANISOU | 30 | O | ASP | C | 5 | 7656 | 12776 | 13293 | −5264 | −611 | 1396 | O |
| ATOM | 31 | CB | ASP | C | 5 | 94.148 | 27.934 | −2.413 | 1.00 | 88.91 | | C |
| ANISOU | 31 | CB | ASP | C | 5 | 8019 | 12128 | 13634 | −5943 | −596 | 1531 | C |
| ATOM | 32 | CG | ASP | C | 5 | 93.437 | 28.173 | −3.750 | 1.00 | 93.23 | | C |
| ANISOU | 32 | CG | ASP | C | 5 | 8751 | 12530 | 14143 | −5961 | −381 | 1777 | C |
| ATOM | 33 | OD1 | ASP | C | 5 | 92.688 | 27.274 | −4.206 | 1.00 | 88.71 | | O |
| ANISOU | 33 | OD1 | ASP | C | 5 | 8327 | 11966 | 13415 | −5645 | −305 | 1826 | O |
| ATOM | 34 | OD2 | ASP | C | 5 | 93.655 | 29.251 | −4.352 | 1.00 | 101.81 | | O |
| ANISOU | 34 | OD2 | ASP | C | 5 | 9837 | 13495 | 15352 | −6300 | −297 | 1923 | O |
| ATOM | 35 | N | LEU | C | 6 | 92.742 | 24.654 | −1.565 | 1.00 | 83.82 | | N |
| ANISOU | 35 | N | LEU | C | 6 | 7621 | 11699 | 12528 | −4925 | −702 | 1374 | N |
| ATOM | 36 | CA | LEU | C | 6 | 92.830 | 23.233 | −1.861 | 1.00 | 83.23 | | C |
| ANISOU | 36 | CA | LEU | C | 6 | 7478 | 11855 | 12290 | −4590 | −670 | 1381 | C |
| ATOM | 37 | C | LEU | C | 6 | 92.335 | 22.886 | −3.260 | 1.00 | 87.17 | | C |
| ANISOU | 37 | C | LEU | C | 6 | 8061 | 12340 | 12719 | −4500 | −450 | 1560 | C |
| ATOM | 38 | O | LEU | C | 6 | 92.488 | 21.751 | −3.713 | 1.00 | 85.67 | | O |
| ANISOU | 38 | O | LEU | C | 6 | 7809 | 12337 | 12405 | −4231 | −406 | 1571 | O |
| ATOM | 39 | CB | LEU | C | 6 | 92.101 | 22.444 | −0.768 | 1.00 | 81.15 | | C |
| ANISOU | 39 | CB | LEU | C | 6 | 7437 | 11496 | 11899 | −4327 | −840 | 1268 | C |
| ATOM | 40 | CG | LEU | C | 6 | 92.882 | 22.093 | 0.526 | 1.00 | 86.87 | | C |
| ANISOU | 40 | CG | LEU | C | 6 | 8000 | 12404 | 12604 | −4256 | −1066 | 1087 | C |
| ATOM | 41 | CD1 | LEU | C | 6 | 94.051 | 23.013 | 0.782 | 1.00 | 89.89 | | C |
| ANISOU | 41 | CD1 | LEU | C | 6 | 8086 | 12919 | 13148 | −4550 | −1123 | 1001 | C |
| ATOM | 42 | CD2 | LEU | C | 6 | 91.982 | 22.148 | 1.707 | 1.00 | 88.12 | | C |
| ANISOU | 42 | CD2 | LEU | C | 6 | 8434 | 12365 | 12682 | −4177 | −1223 | 994 | C |
| ATOM | 43 | N | GLY | C | 7 | 91.809 | 23.890 | −3.953 | 1.00 | 85.70 | | N |
| ANISOU | 43 | N | GLY | C | 7 | 8010 | 11937 | 12615 | −4725 | −327 | 1695 | N |
| ATOM | 44 | CA | GLY | C | 7 | 91.300 | 23.754 | −5.311 | 1.00 | 85.29 | | C |
| ANISOU | 44 | CA | GLY | C | 7 | 8050 | 11857 | 12497 | −4676 | −118 | 1876 | C |
| ATOM | 45 | C | GLY | C | 7 | 89.997 | 22.987 | −5.351 | 1.00 | 86.56 | | C |
| ANISOU | 45 | C | GLY | C | 7 | 8523 | 11842 | 12522 | −4387 | −115 | 1877 | C |
| ATOM | 46 | O | GLY | C | 7 | 89.639 | 22.405 | −6.381 | 1.00 | 85.22 | | O |
| ANISOU | 46 | O | GLY | C | 7 | 8401 | 11725 | 12254 | −4239 | 33 | 1983 | O |
| ATOM | 47 | N | LEU | C | 8 | 89.286 | 22.986 | −4.221 | 1.00 | 81.65 | | N |
| ANISOU | 47 | N | LEU | C | 8 | 8104 | 11034 | 11885 | −4314 | −279 | 1752 | N |
| ATOM | 48 | CA | LEU | C | 8 | 88.022 | 22.288 | −4.100 | 1.00 | 78.46 | | C |
| ANISOU | 48 | CA | LEU | C | 8 | 7988 | 10477 | 11345 | −4076 | −290 | 1736 | C |
| ATOM | 49 | C | LEU | C | 8 | 86.867 | 23.118 | −4.655 | 1.00 | 82.36 | | C |
| ANISOU | 49 | C | LEU | C | 8 | 8743 | 10691 | 11858 | −4154 | −198 | 1824 | C |
| ATOM | 50 | O | LEU | C | 8 | 85.915 | 22.550 | −5.187 | 1.00 | 80.43 | | O |
| ANISOU | 50 | O | LEU | C | 8 | 8669 | 10393 | 11497 | −3975 | −112 | 1874 | O |
| ATOM | 51 | CB | LEU | C | 8 | 87.776 | 21.836 | −2.650 | 1.00 | 77.38 | | C |
| ANISOU | 51 | CB | LEU | C | 8 | 7945 | 10307 | 11147 | −3971 | −493 | 1571 | C |
| ATOM | 52 | CG | LEU | C | 8 | 88.857 | 20.942 | −2.019 | 1.00 | 82.60 | | C |
| ANISOU | 52 | CG | LEU | C | 8 | 8388 | 11232 | 11766 | −3843 | −617 | 1482 | C |
| ATOM | 53 | CD1 | LEU | C | 8 | 88.666 | 20.843 | −0.526 | 1.00 | 82.49 | | C |
| ANISOU | 53 | CD1 | LEU | C | 8 | 8471 | 11168 | 11703 | −3810 | −827 | 1335 | C |
| ATOM | 54 | CD2 | LEU | C | 8 | 88.909 | 19.551 | −2.661 | 1.00 | 82.98 | | C |
| ANISOU | 54 | CD2 | LEU | C | 8 | 8420 | 11427 | 11682 | −3559 | −560 | 1531 | C |
| ATOM | 55 | N | TRP | C | 9 | 86.957 | 24.447 | −4.589 | 1.00 | 81.10 | | N |
| ANISOU | 55 | N | TRP | C | 9 | 8615 | 10353 | 11847 | −4414 | −221 | 1840 | N |
| ATOM | 56 | CA | TRP | C | 9 | 85.886 | 25.261 | −5.133 | 1.00 | 81.21 | | C |
| ANISOU | 56 | CA | TRP | C | 9 | 8887 | 10088 | 11880 | −4461 | −158 | 1919 | C |
| ATOM | 57 | C | TRP | C | 9 | 85.877 | 25.277 | −6.670 | 1.00 | 86.02 | | C |
| ANISOU | 57 | C | TRP | C | 9 | 9477 | 10748 | 12459 | −4471 | 49 | 2129 | C |
| ATOM | 58 | O | TRP | C | 9 | 84.807 | 25.384 | −7.265 | 1.00 | 83.63 | | O |
| ANISOU | 58 | O | TRP | C | 9 | 9397 | 10290 | 12090 | −4370 | 119 | 2192 | O |
| ATOM | 59 | CB | TRP | C | 9 | 85.886 | 26.668 | −4.538 | 1.00 | 82.68 | | C |
| ANISOU | 59 | CB | TRP | C | 9 | 9160 | 10020 | 12234 | −4709 | −275 | 1863 | C |
| ATOM | 60 | CG | TRP | C | 9 | 84.785 | 27.519 | −5.089 | 1.00 | 83.86 | | C |
| ANISOU | 60 | CG | TRP | C | 9 | 9593 | 9868 | 12403 | −4718 | −240 | 1930 | C |
| ATOM | 61 | CD1 | TRP | C | 9 | 84.920 | 28.591 | −5.920 | 1.00 | 88.95 | | C |
| ANISOU | 61 | CD1 | TRP | C | 9 | 10301 | 10333 | 13162 | −4926 | −175 | 2090 | C |
| ATOM | 62 | CD2 | TRP | C | 9 | 83.382 | 27.259 | −4.993 | 1.00 | 81.43 | | C |
| ANISOU | 62 | CD2 | TRP | C | 9 | 9535 | 9434 | 11971 | −4491 | −252 | 1860 | C |
| ATOM | 63 | CE2 | TRP | C | 9 | 82.720 | 28.247 | −5.756 | 1.00 | 86.18 | | C |
| ANISOU | 63 | CE2 | TRP | C | 9 | 10340 | 9777 | 12627 | −4548 | −211 | 1961 | C |
| ATOM | 64 | CE3 | TRP | C | 9 | 82.613 | 26.305 | −4.305 | 1.00 | 80.51 | | C |
| ANISOU | 64 | CE3 | TRP | C | 9 | 9492 | 9402 | 11696 | −4258 | −303 | 1721 | C |
| ATOM | 65 | NE1 | TRP | C | 9 | 83.685 | 29.057 | −6.295 | 1.00 | 87.59 | | N |
| ANISOU | 65 | NE1 | TRP | C | 9 | 10418 | 9908 | 12954 | −4818 | −171 | 2111 | N |
| ATOM | 66 | CZ2 | TRP | C | 9 | 81.328 | 28.308 | −5.857 | 1.00 | 83.82 | | C |
| ANISOU | 66 | CZ2 | TRP | C | 9 | 10289 | 9326 | 12231 | −4356 | −220 | 1902 | C |
| ATOM | 67 | CZ3 | TRP | C | 9 | 81.233 | 26.366 | −4.407 | 1.00 | 80.58 | | C |
| ANISOU | 67 | CZ3 | TRP | C | 9 | 9738 | 9271 | 11607 | −4104 | −297 | 1670 | C |
| ATOM | 68 | CH2 | TRP | C | 9 | 80.604 | 27.358 | −5.174 | 1.00 | 81.86 | | C |
| ANISOU | 68 | CH2 | TRP | C | 9 | 10075 | 9200 | 11828 | −4142 | −258 | 1747 | C |
| ATOM | 69 | N | ASN | C | 10 | 87.060 | 25.135 | −7.308 | 1.00 | 85.59 | | N |
| ANISOU | 69 | N | ASN | C | 10 | 9143 | 10945 | 12434 | −4582 | 146 | 2224 | N |
| ATOM | 70 | CA | ASN | C | 10 | 87.209 | 25.086 | −8.770 | 1.00 | 86.07 | | C |
| ANISOU | 70 | CA | ASN | C | 10 | 9135 | 11129 | 12438 | −4600 | 351 | 2420 | C |
| ATOM | 71 | C | ASN | C | 10 | 86.887 | 23.693 | −9.310 | 1.00 | 88.63 | | C |
| ANISOU | 71 | C | ASN | C | 10 | 9441 | 11651 | 12584 | −4276 | 437 | 2409 | C |
| ATOM | 72 | O | ASN | C | 10 | 86.741 | 23.520 | −10.524 | 1.00 | 89.12 | | O |
| ANISOU | 72 | O | ASN | C | 10 | 9492 | 11806 | 12563 | −4226 | 603 | 2546 | O |
| ATOM | 73 | CB | ASN | C | 10 | 88.614 | 25.506 | −9.188 | 1.00 | 88.63 | | C |
| ANISOU | 73 | CB | ASN | C | 10 | 9140 | 11689 | 12845 | −4863 | 422 | 2506 | C |
| ATOM | 74 | CG | ASN | C | 10 | 88.891 | 26.945 | −8.870 | 1.00 | 123.67 | | C |
| ANISOU | 74 | CG | ASN | C | 10 | 13622 | 15899 | 17468 | −5221 | 349 | 2544 | C |
| ATOM | 75 | ND2 | ASN | C | 10 | 88.437 | 27.836 | −9.743 | 1.00 | 118.82 | | N |
| ANISOU | 75 | ND2 | ASN | C | 10 | 13186 | 15072 | 16888 | −5385 | 438 | 2733 | N |
| ATOM | 76 | OD1 | ASN | C | 10 | 89.438 | 27.277 | −7.811 | 1.00 | 121.68 | | O |
| ANISOU | 76 | OD1 | ASN | C | 10 | 13279 | 15629 | 17327 | −5338 | 193 | 2398 | O |
| ATOM | 77 | N | ARG | C | 11 | 86.778 | 22.707 | −8.401 | 1.00 | 82.66 | | N |
| ANISOU | 77 | N | ARG | C | 11 | 8692 | 10951 | 11765 | −4061 | 315 | 2246 | N |
| ATOM | 78 | CA | ARG | C | 11 | 86.472 | 21.311 | −8.684 | 1.00 | 80.24 | | C |
| ANISOU | 78 | CA | ARG | C | 11 | 8398 | 10784 | 11304 | −3753 | 346 | 2206 | C |
| ATOM | 79 | C | ARG | C | 11 | 84.957 | 21.057 | −8.506 | 1.00 | 81.70 | | C |
| ANISOU | 79 | C | ARG | C | 11 | 8910 | 10732 | 11400 | −3599 | 320 | 2168 | C |
| ATOM | 80 | O | ARG | C | 11 | 84.430 | 20.099 | −9.070 | 1.00 | 79.85 | | O |
| ANISOU | 80 | O | ARG | C | 11 | 8746 | 10553 | 11040 | −3381 | 383 | 2173 | O |
| ATOM | 81 | CB | ARG | C | 11 | 87.318 | 20.424 | −7.757 | 1.00 | 79.87 | | C |
| ANISOU | 81 | CB | ARG | C | 11 | 8171 | 10930 | 11245 | −3634 | 207 | 2065 | C |
| ATOM | 82 | CG | ARG | C | 11 | 87.491 | 18.975 | −8.196 | 1.00 | 86.51 | | C |
| ANISOU | 82 | CG | ARG | C | 11 | 8945 | 11970 | 11956 | −3334 | 231 | 2031 | C |
| ATOM | 83 | CD | ARG | C | 11 | 88.376 | 18.197 | −7.231 | 1.00 | 92.53 | | C |
| ANISOU | 83 | CD | ARG | C | 11 | 9547 | 12895 | 12713 | −3211 | 61 | 1895 | C |
| ATOM | 84 | NE | ARG | C | 11 | 87.832 | 18.141 | −5.869 | 1.00 | 93.76 | | N |
| ANISOU | 84 | NE | ARG | C | 11 | 9897 | 12863 | 12863 | −3203 | −122 | 1799 | N |
| ATOM | 85 | CZ | ARG | C | 11 | 87.134 | 17.122 | −5.374 | 1.00 | 106.26 | | C |
| ANISOU | 85 | CZ | ARG | C | 11 | 11689 | 14355 | 14332 | −2995 | −214 | 1748 | C |
| ATOM | 86 | NH1 | ARG | C | 11 | 86.684 | 17.163 | −4.126 | 1.00 | 97.35 | | N |
| ANISOU | 86 | NH1 | ARG | C | 11 | 10717 | 13093 | 13180 | −3023 | −369 | 1672 | N |
| ATOM | 87 | NH2 | ARG | C | 11 | 86.887 | 16.052 | −6.119 | 1.00 | 89.78 | | N |
| ANISOU | 87 | NH2 | ARG | C | 11 | 9656 | 12314 | 12144 | −2768 | −155 | 1771 | N |
| ATOM | 88 | N | LEU | C | 12 | 84.261 | 21.930 | −7.754 | 1.00 | 78.12 | | N |
| ANISOU | 88 | N | LEU | C | 12 | 8644 | 10030 | 11009 | −3714 | 225 | 2116 | N |
| ATOM | 89 | CA | LEU | C | 12 | 82.820 | 21.831 | −7.514 | 1.00 | 76.14 | | C |
| ANISOU | 89 | CA | LEU | C | 12 | 8674 | 9586 | 10670 | −3593 | 196 | 2057 | C |
| ATOM | 90 | C | LEU | C | 12 | 82.025 | 22.758 | −8.449 | 1.00 | 83.73 | | C |
| ANISOU | 90 | C | LEU | C | 12 | 9797 | 10368 | 11650 | −3655 | 298 | 2165 | C |
| ATOM | 91 | O | LEU | C | 12 | 81.012 | 22.326 | −8.993 | 1.00 | 82.56 | | O |
| ANISOU | 91 | O | LEU | C | 12 | 9804 | 10179 | 11386 | −3494 | 362 | 2171 | O |
| ATOM | 92 | CB | LEU | C | 12 | 82.500 | 22.114 | −6.031 | 1.00 | 75.51 | | C |
| ANISOU | 92 | CB | LEU | C | 12 | 8689 | 9389 | 10614 | −3638 | 13 | 1896 | C |
| ATOM | 93 | CG | LEU | C | 12 | 81.023 | 22.167 | −5.581 | 1.00 | 77.57 | | C |
| ANISOU | 93 | CG | LEU | C | 12 | 9211 | 9481 | 10781 | −3549 | −32 | 1801 | C |
| ATOM | 94 | CD1 | LEU | C | 12 | 80.376 | 20.793 | −5.597 | 1.00 | 75.40 | | C |
| ANISOU | 94 | CD1 | LEU | C | 12 | 9020 | 9299 | 10331 | −3335 | −2 | 1776 | C |
| ATOM | 95 | CD2 | LEU | C | 12 | 80.909 | 22.734 | −4.186 | 1.00 | 79.39 | | C |
| ANISOU | 95 | CD2 | LEU | C | 12 | 9489 | 9628 | 11046 | −3635 | −205 | 1642 | C |
| ATOM | 96 | N | GLU | C | 13 | 82.494 | 24.016 | −8.639 | 1.00 | 84.24 | | N |
| ANISOU | 96 | N | GLU | C | 13 | 9827 | 10323 | 11855 | −3888 | 303 | 2251 | N |
| ATOM | 97 | CA | GLU | C | 13 | 81.921 | 25.083 | −9.483 | 1.00 | 85.49 | | C |
| ANISOU | 97 | CA | GLU | C | 13 | 10150 | 10278 | 12056 | −3980 | 369 | 2378 | C |
| ATOM | 98 | C | GLU | C | 13 | 81.268 | 24.586 | −10.816 | 1.00 | 90.60 | | C |
| ANISOU | 98 | C | GLU | C | 13 | 10871 | 10993 | 12561 | −3810 | 534 | 2499 | C |
| ATOM | 99 | O | GLU | C | 13 | 80.122 | 24.972 | −11.062 | 1.00 | 89.01 | | O |
| ANISOU | 99 | O | GLU | C | 13 | 10896 | 10610 | 12316 | −3725 | 527 | 2492 | O |
| ATOM | 100 | CB | GLU | C | 13 | 82.998 | 26.172 | −9.747 | 1.00 | 90.06 | | C |
| ANISOU | 100 | CB | GLU | C | 13 | 10603 | 10822 | 12795 | −4286 | 384 | 2509 | C |
| ATOM | 101 | CG | GLU | C | 13 | 82.672 | 27.231 | −10.788 | 1.00 | 102.96 | | C |
| ANISOU | 101 | CG | GLU | C | 13 | 12379 | 12279 | 14462 | −4411 | 472 | 2709 | C |
| ATOM | 102 | CD | GLU | C | 13 | 81.602 | 28.228 | −10.392 | 1.00 | 130.04 | | C |
| ANISOU | 102 | CD | GLU | C | 13 | 16108 | 15340 | 17960 | −4422 | 347 | 2655 | C |
| ATOM | 103 | OE1 | GLU | C | 13 | 80.518 | 28.212 | −11.020 | 1.00 | 127.82 | | O |
| ANISOU | 103 | OE1 | GLU | C | 13 | 16025 | 14956 | 17584 | −4263 | 395 | 2704 | O |
| ATOM | 104 | OE2 | GLU | C | 13 | 81.846 | 29.032 | −9.462 | 1.00 | 129.42 | | O |
| ANISOU | 104 | OE2 | GLU | C | 13 | 16061 | 15085 | 18028 | −4577 | 191 | 2549 | O |
| ATOM | 105 | N | PRO | C | 14 | 81.933 | 23.760 | −11.684 | 1.00 | 89.49 | | N |
| ANISOU | 105 | N | PRO | C | 14 | 10541 | 11122 | 12339 | −3740 | 670 | 2589 | N |
| ATOM | 106 | CA | PRO | C | 14 | 81.290 | 23.354 | −12.951 | 1.00 | 88.26 | | C |
| ANISOU | 106 | CA | PRO | C | 14 | 10455 | 11035 | 12044 | −3580 | 819 | 2689 | C |
| ATOM | 107 | C | PRO | C | 14 | 80.186 | 22.315 | −12.819 | 1.00 | 87.58 | | C |
| ANISOU | 107 | C | PRO | C | 14 | 10506 | 10950 | 11820 | −3312 | 802 | 2558 | C |
| ATOM | 108 | O | PRO | C | 14 | 79.332 | 22.235 | −13.701 | 1.00 | 87.59 | | O |
| ANISOU | 108 | O | PRO | C | 14 | 10632 | 10930 | 11719 | −3190 | 887 | 2608 | O |
| ATOM | 109 | CB | PRO | C | 14 | 82.453 | 22.802 | −13.789 | 1.00 | 91.83 | | C |
| ANISOU | 109 | CB | PRO | C | 14 | 10630 | 11806 | 12454 | −3592 | 951 | 2786 | C |
| ATOM | 110 | CG | PRO | C | 14 | 83.705 | 23.160 | −13.042 | 1.00 | 98.98 | | C |
| ANISOU | 110 | CG | PRO | C | 14 | 11325 | 12784 | 13498 | −3807 | 881 | 2767 | C |
| ATOM | 111 | CD | PRO | C | 14 | 83.299 | 23.202 | −11.612 | 1.00 | 93.25 | | C |
| ANISOU | 111 | CD | PRO | C | 14 | 10719 | 11872 | 12841 | −3793 | 694 | 2591 | C |
| ATOM | 112 | N | ALA | C | 15 | 80.209 | 21.505 | −11.755 | 1.00 | 79.95 | | N |
| ANISOU | 112 | N | ALA | C | 15 | 9516 | 10022 | 10840 | −3230 | 692 | 2399 | N |
| ATOM | 113 | CA | ALA | C | 15 | 79.170 | 20.511 | −11.536 | 1.00 | 76.56 | | C |
| ANISOU | 113 | CA | ALA | C | 15 | 9224 | 9585 | 10280 | −3021 | 668 | 2282 | C |
| ATOM | 114 | C | ALA | C | 15 | 77.890 | 21.229 | −11.092 | 1.00 | 78.78 | | C |
| ANISOU | 114 | C | ALA | C | 15 | 9736 | 9646 | 10551 | −3029 | 601 | 2205 | C |
| ATOM | 115 | O | ALA | C | 15 | 76.795 | 20.685 | −11.213 | 1.00 | 76.83 | | O |
| ANISOU | 115 | O | ALA | C | 15 | 9621 | 9388 | 10183 | −2882 | 615 | 2131 | O |
| ATOM | 116 | CB | ALA | C | 15 | 79.619 | 19.511 | −10.487 | 1.00 | 76.79 | | C |
| ANISOU | 116 | CB | ALA | C | 15 | 9181 | 9703 | 10293 | −2961 | 557 | 2162 | C |
| ATOM | 117 | N | LEU | C | 16 | 78.042 | 22.477 | −10.639 | 1.00 | 76.96 | | N |
| ANISOU | 117 | N | LEU | C | 16 | 9545 | 9249 | 10447 | −3200 | 526 | 2213 | N |
| ATOM | 118 | CA | LEU | C | 16 | 76.981 | 23.362 | −10.167 | 1.00 | 77.09 | | C |
| ANISOU | 118 | CA | LEU | C | 16 | 9763 | 9051 | 10478 | −3206 | 436 | 2118 | C |
| ATOM | 119 | C | LEU | C | 16 | 76.324 | 24.157 | −11.312 | 1.00 | 84.28 | | C |
| ANISOU | 119 | C | LEU | C | 16 | 10812 | 9841 | 11370 | −3167 | 513 | 2228 | C |
| ATOM | 120 | O | LEU | C | 16 | 75.358 | 24.880 | −11.057 | 1.00 | 84.66 | | O |
| ANISOU | 120 | O | LEU | C | 16 | 11035 | 9726 | 11406 | −3113 | 439 | 2133 | O |
| ATOM | 121 | CB | LEU | C | 16 | 77.591 | 24.342 | −9.143 | 1.00 | 78.47 | | C |
| ANISOU | 121 | CB | LEU | C | 16 | 9918 | 9087 | 10812 | −3399 | 297 | 2062 | C |
| ATOM | 122 | CG | LEU | C | 16 | 77.342 | 24.144 | −7.650 | 1.00 | 81.39 | | C |
| ANISOU | 122 | CG | LEU | C | 16 | 10292 | 9463 | 11171 | −3401 | 146 | 1862 | C |
| ATOM | 123 | CD1 | LEU | C | 16 | 77.487 | 22.701 | −7.222 | 1.00 | 79.31 | | C |
| ANISOU | 123 | CD1 | LEU | C | 16 | 9965 | 9399 | 10769 | −3273 | 162 | 1808 | C |
| ATOM | 124 | CD2 | LEU | C | 16 | 78.293 | 24.996 | −6.859 | 1.00 | 85.37 | | C |
| ANISOU | 124 | CD2 | LEU | C | 16 | 10678 | 9925 | 11835 | −3600 | 44 | 1848 | C |
| ATOM | 125 | N | ALA | C | 17 | 76.841 | 24.031 | −12.562 | 1.00 | 82.26 | | N |
| ANISOU | 125 | N | ALA | C | 17 | 10475 | 9681 | 11099 | −3184 | 654 | 2420 | N |
| ATOM | 126 | CA | ALA | C | 17 | 76.357 | 24.704 | −13.785 | 1.00 | 82.80 | | C |
| ANISOU | 126 | CA | ALA | C | 17 | 10666 | 9667 | 11128 | −3151 | 738 | 2567 | C |
| ATOM | 127 | C | ALA | C | 17 | 74.840 | 24.938 | −13.807 | 1.00 | 84.29 | | C |
| ANISOU | 127 | C | ALA | C | 17 | 11070 | 9734 | 11222 | −2968 | 690 | 2451 | C |
| ATOM | 128 | O | ALA | C | 17 | 74.387 | 26.078 | −13.945 | 1.00 | 84.80 | | O |
| ANISOU | 128 | O | ALA | C | 17 | 11302 | 9578 | 11339 | −2992 | 624 | 2482 | O |
| ATOM | 129 | CB | ALA | C | 17 | 76.772 | 23.906 | −15.016 | 1.00 | 83.63 | | C |
| ANISOU | 129 | CB | ALA | C | 17 | 10636 | 10012 | 11126 | −3084 | 912 | 2712 | C |
| ATOM | 130 | N | TYR | C | 18 | 74.074 | 23.855 | −13.587 | 1.00 | 78.00 | | N |
| ANISOU | 130 | N | TYR | C | 18 | 10269 | 9077 | 10290 | −2793 | 707 | 2304 | N |
| ATOM | 131 | CA | TYR | C | 18 | 72.613 | 23.798 | −13.567 | 1.00 | 76.09 | | C |
| ANISOU | 131 | CA | TYR | C | 18 | 10180 | 8802 | 9929 | −2615 | 676 | 2158 | C |
| ATOM | 132 | C | TYR | C | 18 | 71.921 | 24.759 | −12.580 | 1.00 | 83.11 | | C |
| ANISOU | 132 | C | TYR | C | 18 | 11207 | 9496 | 10876 | −2629 | 517 | 1997 | C |
| ATOM | 133 | O | TYR | C | 18 | 70.684 | 24.805 | −12.568 | 1.00 | 82.22 | | O |
| ANISOU | 133 | O | TYR | C | 18 | 11201 | 9380 | 10658 | −2476 | 485 | 1855 | O |
| ATOM | 134 | CB | TYR | C | 18 | 72.149 | 22.352 | −13.309 | 1.00 | 73.54 | | C |
| ANISOU | 134 | CB | TYR | C | 18 | 9798 | 8675 | 9467 | −2493 | 713 | 2028 | C |
| ATOM | 135 | CG | TYR | C | 18 | 72.402 | 21.850 | −11.904 | 1.00 | 72.47 | | C |
| ANISOU | 135 | CG | TYR | C | 18 | 9611 | 8566 | 9357 | −2568 | 614 | 1890 | C |
| ATOM | 136 | CD1 | TYR | C | 18 | 71.531 | 22.165 | −10.861 | 1.00 | 73.49 | | C |
| ANISOU | 136 | CD1 | TYR | C | 18 | 9834 | 8633 | 9457 | −2564 | 501 | 1703 | C |
| ATOM | 137 | CD2 | TYR | C | 18 | 73.470 | 21.002 | −11.629 | 1.00 | 72.45 | | C |
| ANISOU | 137 | CD2 | TYR | C | 18 | 9466 | 8678 | 9386 | −2624 | 629 | 1936 | C |
| ATOM | 138 | CE1 | TYR | C | 18 | 71.747 | 21.695 | −9.569 | 1.00 | 72.60 | | C |
| ANISOU | 138 | CE1 | TYR | C | 18 | 9678 | 8566 | 9338 | −2638 | 412 | 1588 | C |
| ATOM | 139 | CE2 | TYR | C | 18 | 73.688 | 20.515 | −10.341 | 1.00 | 72.64 | | C |
| ANISOU | 139 | CE2 | TYR | C | 18 | 9460 | 8726 | 9414 | −2681 | 526 | 1823 | C |
| ATOM | 140 | CZ | TYR | C | 18 | 72.818 | 20.857 | −9.315 | 1.00 | 77.46 | | C |
| ANISOU | 140 | CZ | TYR | C | 18 | 10173 | 9274 | 9986 | −2697 | 422 | 1659 | C |
| ATOM | 141 | OH | TYR | C | 18 | 73.008 | 20.370 | −8.045 | 1.00 | 74.91 | | O |
| ANISOU | 141 | OH | TYR | C | 18 | 9827 | 8998 | 9639 | −2758 | 323 | 1559 | O |
| ATOM | 142 | N | LEU | C | 19 | 72.694 | 25.472 | −11.728 | 1.00 | 82.61 | | N |
| ANISOU | 142 | N | LEU | C | 19 | 11124 | 9295 | 10970 | −2803 | 413 | 1992 | N |
| ATOM | 143 | CA | LEU | C | 19 | 72.114 | 26.383 | −10.740 | 1.00 | 83.83 | | C |
| ANISOU | 143 | CA | LEU | C | 19 | 11397 | 9270 | 11185 | −2809 | 247 | 1812 | C |
| ATOM | 144 | C | LEU | C | 19 | 71.851 | 27.760 | −11.285 | 1.00 | 91.51 | | C |
| ANISOU | 144 | C | LEU | C | 19 | 12543 | 9980 | 12248 | −2808 | 179 | 1884 | C |
| ATOM | 145 | O | LEU | C | 19 | 72.515 | 28.191 | −12.227 | 1.00 | 92.28 | | O |
| ANISOU | 145 | O | LEU | C | 19 | 12653 | 10002 | 12409 | −2900 | 249 | 2118 | O |
| ATOM | 146 | CB | LEU | C | 19 | 72.951 | 26.480 | −9.455 | 1.00 | 84.41 | | C |
| ANISOU | 146 | CB | LEU | C | 19 | 11377 | 9322 | 11373 | −2977 | 139 | 1727 | C |
| ATOM | 147 | CG | LEU | C | 19 | 72.871 | 25.327 | −8.450 | 1.00 | 87.52 | | C |
| ANISOU | 147 | CG | LEU | C | 19 | 11663 | 9925 | 11664 | −2956 | 129 | 1585 | C |
| ATOM | 148 | CD1 | LEU | C | 19 | 73.822 | 25.566 | −7.308 | 1.00 | 88.78 | | C |
| ANISOU | 148 | CD1 | LEU | C | 19 | 11725 | 10063 | 11944 | −3125 | 22 | 1540 | C |
| ATOM | 149 | CD2 | LEU | C | 19 | 71.458 | 25.157 | −7.878 | 1.00 | 89.13 | | C |
| ANISOU | 149 | CD2 | LEU | C | 19 | 11959 | 10186 | 11721 | −2814 | 70 | 1355 | C |
| ATOM | 150 | N | ALA | C | 20 | 70.870 | 28.451 | −10.676 | 1.00 | 90.53 | | N |
| ANISOU | 150 | N | ALA | C | 20 | 12555 | 9723 | 12118 | −2700 | 35 | 1677 | N |
| ATOM | 151 | CA | ALA | C | 20 | 70.484 | 29.817 | −11.012 | 1.00 | 93.75 | | C |
| ANISOU | 151 | CA | ALA | C | 20 | 13166 | 9836 | 12618 | −2661 | −85 | 1692 | C |
| ATOM | 152 | C | ALA | C | 20 | 71.581 | 30.794 | −10.491 | 1.00 | 102.95 | | C |
| ANISOU | 152 | C | ALA | C | 20 | 14347 | 10760 | 14008 | −2907 | −195 | 1762 | C |
| ATOM | 153 | O | ALA | C | 20 | 72.322 | 30.415 | −9.576 | 1.00 | 102.55 | | O |
| ANISOU | 153 | O | ALA | C | 20 | 14149 | 10805 | 14011 | −3045 | −216 | 1688 | O |
| ATOM | 154 | CB | ALA | C | 20 | 69.129 | 30.136 | −10.386 | 1.00 | 94.05 | | C |
| ANISOU | 154 | CB | ALA | C | 20 | 13308 | 9855 | 12571 | −2447 | −220 | 1394 | C |
| ATOM | 155 | N | PRO | C | 21 | 71.742 | 32.018 | −11.067 | 1.00 | 103.25 | | N |
| ANISOU | 155 | N | PRO | C | 21 | 14562 | 10490 | 14177 | −2980 | −270 | 1914 | N |
| ATOM | 156 | CA | PRO | C | 21 | 72.782 | 32.940 | −10.574 | 1.00 | 105.99 | | C |
| ANISOU | 156 | CA | PRO | C | 21 | 14925 | 10600 | 14747 | −3248 | −379 | 1977 | C |
| ATOM | 157 | C | PRO | C | 21 | 72.882 | 33.043 | −9.051 | 1.00 | 110.40 | | C |
| ANISOU | 157 | C | PRO | C | 21 | 15415 | 11151 | 15381 | −3293 | −532 | 1696 | C |
| ATOM | 158 | O | PRO | C | 21 | 73.947 | 32.772 | −8.497 | 1.00 | 109.49 | | O |
| ANISOU | 158 | O | PRO | C | 21 | 15126 | 11130 | 15346 | −3500 | −511 | 1723 | O |
| ATOM | 159 | CB | PRO | C | 21 | 72.389 | 34.288 | −11.207 | 1.00 | 110.85 | | C |
| ANISOU | 159 | CB | PRO | C | 21 | 15827 | 10832 | 15460 | −3227 | −506 | 2080 | C |
| ATOM | 160 | CG | PRO | C | 21 | 70.994 | 34.080 | −11.772 | 1.00 | 113.94 | | C |
| ANISOU | 160 | CG | PRO | C | 21 | 16342 | 11283 | 15668 | −2896 | −493 | 2002 | C |
| ATOM | 161 | CD | PRO | C | 21 | 70.978 | 32.644 | −12.162 | 1.00 | 106.23 | | C |
| ANISOU | 161 | CD | PRO | C | 21 | 15150 | 10707 | 14507 | −2830 | −276 | 2037 | C |
| ATOM | 162 | N | GLU | C | 22 | 71.761 | 33.394 | −8.388 | 1.00 | 108.29 | | N |
| ANISOU | 162 | N | GLU | C | 22 | 15267 | 10811 | 15067 | −3083 | −685 | 1413 | N |
| ATOM | 163 | CA | GLU | C | 22 | 71.641 | 33.526 | −6.926 | 1.00 | 108.68 | | C |
| ANISOU | 163 | CA | GLU | C | 22 | 15265 | 10883 | 15145 | −3078 | −841 | 1105 | C |
| ATOM | 164 | C | GLU | C | 22 | 71.744 | 32.185 | −6.198 | 1.00 | 108.29 | | C |
| ANISOU | 164 | C | GLU | C | 22 | 14988 | 11216 | 14943 | −3073 | −737 | 1003 | C |
| ATOM | 165 | O | GLU | C | 22 | 72.256 | 32.142 | −5.077 | 1.00 | 107.73 | | O |
| ANISOU | 165 | O | GLU | C | 22 | 14814 | 11202 | 14917 | −3179 | −823 | 858 | O |
| ATOM | 166 | CB | GLU | C | 22 | 70.360 | 34.292 | −6.492 | 1.00 | 111.44 | | C |
| ANISOU | 166 | CB | GLU | C | 22 | 15793 | 11089 | 15461 | −2829 | −1030 | 814 | C |
| ATOM | 167 | CG | GLU | C | 22 | 69.247 | 34.447 | −7.529 | 1.00 | 124.29 | | C |
| ANISOU | 167 | CG | GLU | C | 22 | 17579 | 12671 | 16976 | −2574 | −1005 | 848 | C |
| ATOM | 168 | CD | GLU | C | 22 | 68.521 | 33.195 | −7.994 | 1.00 | 141.09 | | C |
| ANISOU | 168 | CD | GLU | C | 22 | 19587 | 15156 | 18864 | −2421 | −811 | 865 | C |
| ATOM | 169 | OE1 | GLU | C | 22 | 68.188 | 33.130 | −9.198 | 1.00 | 132.91 | | O |
| ANISOU | 169 | OE1 | GLU | C | 22 | 18641 | 14092 | 17768 | −2318 | −725 | 1041 | O |
| ATOM | 170 | OE2 | GLU | C | 22 | 68.274 | 32.289 | −7.164 | 1.00 | 131.22 | | O |
| ANISOU | 170 | OE2 | GLU | C | 22 | 18165 | 14210 | 17484 | −2412 | −752 | 707 | O |
| ATOM | 171 | N | GLU | C | 23 | 71.252 | 31.102 | −6.835 | 1.00 | 101.60 | | N |
| ANISOU | 171 | N | GLU | C | 23 | 14075 | 10617 | 13911 | −2949 | −565 | 1079 | N |
| ATOM | 172 | CA | GLU | C | 23 | 71.306 | 29.742 | −6.300 | 1.00 | 98.85 | | C |
| ANISOU | 172 | CA | GLU | C | 23 | 13547 | 10600 | 13410 | −2944 | −464 | 1021 | C |
| ATOM | 173 | C | GLU | C | 23 | 72.776 | 29.248 | −6.221 | 1.00 | 100.99 | | C |
| ANISOU | 173 | C | GLU | C | 23 | 13651 | 10944 | 13776 | −3165 | −395 | 1201 | C |
| ATOM | 174 | O | GLU | C | 23 | 73.145 | 28.579 | −5.253 | 1.00 | 99.47 | | O |
| ANISOU | 174 | O | GLU | C | 23 | 13330 | 10924 | 13540 | −3220 | −418 | 1103 | O |
| ATOM | 175 | CB | GLU | C | 23 | 70.427 | 28.778 | −7.138 | 1.00 | 98.39 | | C |
| ANISOU | 175 | CB | GLU | C | 23 | 13485 | 10745 | 13153 | −2767 | −311 | 1064 | C |
| ATOM | 176 | CG | GLU | C | 23 | 68.929 | 28.793 | −6.809 | 1.00 | 109.36 | | C |
| ANISOU | 176 | CG | GLU | C | 23 | 14947 | 12222 | 14381 | −2552 | −368 | 805 | C |
| ATOM | 177 | CD | GLU | C | 23 | 68.134 | 27.522 | −7.095 | 1.00 | 122.36 | | C |
| ANISOU | 177 | CD | GLU | C | 23 | 16522 | 14164 | 15807 | −2441 | −233 | 768 | C |
| ATOM | 178 | OE1 | GLU | C | 23 | 68.457 | 26.819 | −8.080 | 1.00 | 117.63 | | O |
| ANISOU | 178 | OE1 | GLU | C | 23 | 15878 | 13639 | 15176 | −2452 | −84 | 971 | O |
| ATOM | 179 | OE2 | GLU | C | 23 | 67.187 | 27.223 | −6.327 | 1.00 | 105.36 | | O |
| ANISOU | 179 | OE2 | GLU | C | 23 | 14350 | 12178 | 13505 | −2354 | −276 | 526 | O |
| ATOM | 180 | N | ARG | C | 24 | 73.610 | 29.601 | −7.227 | 1.00 | 97.56 | | N |
| ANISOU | 180 | N | ARG | C | 24 | 13213 | 10395 | 13460 | −3291 | −318 | 1459 | N |
| ATOM | 181 | CA | ARG | C | 24 | 75.034 | 29.234 | −7.276 | 1.00 | 97.09 | | C |
| ANISOU | 181 | CA | ARG | C | 24 | 12970 | 10426 | 13491 | −3498 | −248 | 1625 | C |
| ATOM | 182 | C | ARG | C | 24 | 75.824 | 30.032 | −6.223 | 1.00 | 102.07 | | C |
| ANISOU | 182 | C | ARG | C | 24 | 13559 | 10926 | 14296 | −3693 | −410 | 1523 | C |
| ATOM | 183 | O | ARG | C | 24 | 76.714 | 29.478 | −5.575 | 1.00 | 101.74 | | O |
| ANISOU | 183 | O | ARG | C | 24 | 13338 | 11053 | 14267 | −3792 | −414 | 1496 | O |
| ATOM | 184 | CB | ARG | C | 24 | 75.617 | 29.448 | −8.691 | 1.00 | 95.93 | | C |
| ANISOU | 184 | CB | ARG | C | 24 | 12821 | 10232 | 13394 | −3587 | −110 | 1920 | C |
| ATOM | 185 | CG | ARG | C | 24 | 76.183 | 28.176 | −9.336 | 1.00 | 96.36 | | C |
| ANISOU | 185 | CG | ARG | C | 24 | 12690 | 10583 | 13338 | −3559 | 75 | 2059 | C |
| ATOM | 186 | CD | ARG | C | 24 | 76.434 | 28.315 | −10.834 | 1.00 | 103.13 | | C |
| ANISOU | 186 | CD | ARG | C | 24 | 13555 | 11445 | 14182 | −3592 | 225 | 2324 | C |
| ATOM | 187 | NE | ARG | C | 24 | 75.282 | 28.871 | −11.552 | 1.00 | 111.48 | | N |
| ANISOU | 187 | NE | ARG | C | 24 | 14836 | 12351 | 15172 | −3434 | 224 | 2347 | N |
| ATOM | 188 | CZ | ARG | C | 24 | 75.366 | 29.711 | −12.582 | 1.00 | 127.05 | | C |
| ANISOU | 188 | CZ | ARG | C | 24 | 16924 | 14169 | 17180 | −3498 | 264 | 2560 | C |
| ATOM | 189 | NH1 | ARG | C | 24 | 76.551 | 30.091 | −13.045 | 1.00 | 118.18 | | N |
| ANISOU | 189 | NH1 | ARG | C | 24 | 15705 | 13040 | 16158 | −3747 | 328 | 2778 | N |
| ATOM | 190 | NH2 | ARG | C | 24 | 74.265 | 30.177 | −13.158 | 1.00 | 111.00 | | N |
| ANISOU | 190 | NH2 | ARG | C | 24 | 15100 | 12003 | 15071 | −3319 | 239 | 2559 | N |
| ATOM | 191 | N | ALA | C | 25 | 75.467 | 31.323 | −6.041 | 1.00 | 99.27 | | N |
| ANISOU | 191 | N | ALA | C | 25 | 13379 | 10268 | 14072 | −3728 | −560 | 1448 | N |
| ATOM | 192 | CA | ALA | C | 25 | 76.074 | 32.244 | −5.077 | 1.00 | 100.44 | | C |
| ANISOU | 192 | CA | ALA | C | 25 | 13521 | 10241 | 14398 | −3905 | −741 | 1321 | C |
| ATOM | 193 | C | ALA | C | 25 | 75.720 | 31.849 | −3.646 | 1.00 | 100.11 | | C |
| ANISOU | 193 | C | ALA | C | 25 | 13409 | 10361 | 14268 | −3817 | −854 | 1026 | C |
| ATOM | 194 | O | ALA | C | 25 | 76.524 | 32.062 | −2.737 | 1.00 | 100.90 | | O |
| ANISOU | 194 | O | ALA | C | 25 | 13398 | 10475 | 14464 | −3972 | −955 | 937 | O |
| ATOM | 195 | CB | ALA | C | 25 | 75.606 | 33.662 | −5.355 | 1.00 | 103.88 | | C |
| ANISOU | 195 | CB | ALA | C | 25 | 14204 | 10289 | 14977 | −3913 | −888 | 1297 | C |
| ATOM | 196 | N | LYS | C | 26 | 74.512 | 31.275 | −3.455 | 1.00 | 92.28 | | N |
| ANISOU | 196 | N | LYS | C | 26 | 12473 | 9511 | 13079 | −3580 | −834 | 875 | N |
| ATOM | 197 | CA | LYS | C | 26 | 73.981 | 30.774 | −2.183 | 1.00 | 89.83 | | C |
| ANISOU | 197 | CA | LYS | C | 26 | 12102 | 9406 | 12623 | −3486 | −913 | 611 | C |
| ATOM | 198 | C | LYS | C | 26 | 74.923 | 29.683 | −1.672 | 1.00 | 89.28 | | C |
| ANISOU | 198 | C | LYS | C | 26 | 11830 | 9595 | 12498 | −3590 | −846 | 683 | C |
| ATOM | 199 | O | LYS | C | 26 | 75.303 | 29.702 | −0.506 | 1.00 | 89.13 | | O |
| ANISOU | 199 | O | LYS | C | 26 | 11729 | 9656 | 12482 | −3656 | −963 | 528 | O |
| ATOM | 200 | CB | LYS | C | 26 | 72.573 | 30.195 | −2.419 | 1.00 | 91.03 | | C |
| ANISOU | 200 | CB | LYS | C | 26 | 12328 | 9704 | 12554 | −3247 | −846 | 511 | C |
| ATOM | 201 | CG | LYS | C | 26 | 71.640 | 30.195 | −1.212 | 1.00 | 112.17 | | C |
| ANISOU | 201 | CG | LYS | C | 26 | 15010 | 12524 | 15086 | −3133 | −964 | 190 | C |
| ATOM | 202 | CD | LYS | C | 26 | 70.210 | 29.780 | −1.619 | 1.00 | 120.77 | | C |
| ANISOU | 202 | CD | LYS | C | 26 | 16170 | 13744 | 15974 | −2912 | −898 | 89 | C |
| ATOM | 203 | CE | LYS | C | 26 | 69.216 | 29.810 | −0.477 | 1.00 | 124.60 | | C |
| ANISOU | 203 | CE | LYS | C | 26 | 16629 | 14437 | 16278 | −2809 | −991 | −233 | C |
| ATOM | 204 | NZ | LYS | C | 26 | 69.322 | 28.610 | 0.389 | 1.00 | 124.65 | | N |
| ANISOU | 204 | NZ | LYS | C | 26 | 16501 | 14761 | 16097 | −2882 | −914 | −227 | N |
| ATOM | 205 | N | VAL | C | 27 | 75.340 | 28.773 | −2.577 | 1.00 | 82.94 | | N |
| ANISOU | 205 | N | VAL | C | 27 | 10947 | 8916 | 11649 | −3593 | −672 | 914 | N |
| ATOM | 206 | CA | VAL | C | 27 | 76.260 | 27.651 | −2.342 | 1.00 | 80.96 | | C |
| ANISOU | 206 | CA | VAL | C | 27 | 10514 | 8898 | 11349 | −3652 | −603 | 1009 | C |
| ATOM | 207 | C | VAL | C | 27 | 77.691 | 28.142 | −2.018 | 1.00 | 86.09 | | C |
| ANISOU | 207 | C | VAL | C | 27 | 11020 | 9502 | 12189 | −3870 | −671 | 1064 | C |
| ATOM | 208 | O | VAL | C | 27 | 78.307 | 27.615 | −1.091 | 1.00 | 86.09 | | O |
| ANISOU | 208 | O | VAL | C | 27 | 10886 | 9668 | 12158 | −3912 | −733 | 996 | O |
| ATOM | 209 | CB | VAL | C | 27 | 76.217 | 26.629 | −3.515 | 1.00 | 82.38 | | C |
| ANISOU | 209 | CB | VAL | C | 27 | 10665 | 9205 | 11432 | −3561 | −411 | 1211 | C |
| ATOM | 210 | CG1 | VAL | C | 27 | 77.226 | 25.505 | −3.324 | 1.00 | 81.29 | | C |
| ANISOU | 210 | CG1 | VAL | C | 27 | 10345 | 9283 | 11257 | −3595 | −364 | 1297 | C |
| ATOM | 211 | CG2 | VAL | C | 27 | 74.817 | 26.054 | −3.681 | 1.00 | 80.19 | | C |
| ANISOU | 211 | CG2 | VAL | C | 27 | 10506 | 9003 | 10960 | −3366 | −358 | 1127 | C |
| ATOM | 212 | N | ARG | C | 28 | 78.207 | 29.150 | −2.766 | 1.00 | 83.25 | | N |
| ANISOU | 212 | N | ARG | C | 28 | 10693 | 8924 | 12016 | −4017 | −667 | 1188 | N |
| ATOM | 213 | CA | ARG | C | 28 | 79.538 | 29.744 | −2.552 | 1.00 | 84.82 | | C |
| ANISOU | 213 | CA | ARG | C | 28 | 10752 | 9070 | 12407 | −4265 | −727 | 1240 | C |
| ATOM | 214 | C | ARG | C | 28 | 79.715 | 30.099 | −1.058 | 1.00 | 89.38 | | C |
| ANISOU | 214 | C | ARG | C | 28 | 11296 | 9641 | 13025 | −4318 | −929 | 986 | C |
| ATOM | 215 | O | ARG | C | 28 | 80.716 | 29.720 | −0.442 | 1.00 | 89.27 | | O |
| ANISOU | 215 | O | ARG | C | 28 | 11090 | 9787 | 13043 | −4424 | −972 | 962 | O |
| ATOM | 216 | CB | ARG | C | 28 | 79.728 | 30.990 | −3.450 | 1.00 | 85.60 | | C |
| ANISOU | 216 | CB | ARG | C | 28 | 10961 | 8878 | 12687 | −4428 | −718 | 1392 | C |
| ATOM | 217 | CG | ARG | C | 28 | 81.188 | 31.322 | −3.782 | 1.00 | 92.07 | | C |
| ANISOU | 217 | CG | ARG | C | 28 | 11593 | 9720 | 13668 | −4713 | −682 | 1555 | C |
| ATOM | 218 | CD | ARG | C | 28 | 81.804 | 32.391 | −2.883 | 1.00 | 93.77 | | C |
| ANISOU | 218 | CD | ARG | C | 28 | 11805 | 9736 | 14087 | −4943 | −873 | 1426 | C |
| ATOM | 219 | NE | ARG | C | 28 | 83.264 | 32.273 | −2.851 | 1.00 | 86.55 | | N |
| ANISOU | 219 | NE | ARG | C | 28 | 10623 | 8986 | 13276 | −5191 | −845 | 1510 | N |
| ATOM | 220 | CZ | ARG | C | 28 | 84.092 | 33.131 | −2.263 | 1.00 | 81.29 | | C |
| ANISOU | 220 | CZ | ARG | C | 28 | 9885 | 8208 | 12794 | −5443 | −988 | 1424 | C |
| ATOM | 221 | NH1 | ARG | C | 28 | 83.619 | 34.205 | −1.643 | 1.00 | 57.80 | | N |
| ANISOU | 221 | NH1 | ARG | C | 28 | 7103 | 4923 | 9934 | −5477 | −1180 | 1246 | N |
| ATOM | 222 | NH2 | ARG | C | 28 | 85.400 | 32.918 | −2.286 | 1.00 | 59.00 | | N |
| ANISOU | 222 | NH2 | ARG | C | 28 | 6785 | 5591 | 10042 | −5657 | −947 | 1494 | N |
| ATOM | 223 | N | GLU | C | 29 | 78.685 | 30.757 | −0.481 | 1.00 | 86.07 | | N |
| ANISOU | 223 | N | GLU | C | 29 | 11053 | 9070 | 12582 | −4215 | −1054 | 779 | N |
| ATOM | 224 | CA | GLU | C | 29 | 78.577 | 31.175 | 0.917 | 1.00 | 86.92 | | C |
| ANISOU | 224 | CA | GLU | C | 29 | 11157 | 9174 | 12694 | −4222 | −1251 | 499 | C |
| ATOM | 225 | C | GLU | C | 29 | 78.636 | 29.959 | 1.858 | 1.00 | 87.40 | | C |
| ANISOU | 225 | C | GLU | C | 29 | 11083 | 9571 | 12554 | −4135 | −1251 | 409 | C |
| ATOM | 226 | O | GLU | C | 29 | 79.256 | 30.043 | 2.925 | 1.00 | 88.52 | | O |
| ANISOU | 226 | O | GLU | C | 29 | 11115 | 9798 | 12720 | −4219 | −1384 | 269 | O |
| ATOM | 227 | CB | GLU | C | 29 | 77.253 | 31.931 | 1.122 | 1.00 | 88.77 | | C |
| ANISOU | 227 | CB | GLU | C | 29 | 11607 | 9231 | 12890 | −4063 | −1351 | 297 | C |
| ATOM | 228 | CG | GLU | C | 29 | 77.395 | 33.378 | 1.554 | 1.00 | 104.99 | | C |
| ANISOU | 228 | CG | GLU | C | 29 | 13753 | 10995 | 15142 | −4165 | −1563 | 114 | C |
| ATOM | 229 | CD | GLU | C | 29 | 76.080 | 33.981 | 2.008 | 1.00 | 131.40 | | C |
| ANISOU | 229 | CD | GLU | C | 29 | 17281 | 14228 | 18417 | −3954 | −1689 | −150 | C |
| ATOM | 230 | OE1 | GLU | C | 29 | 75.202 | 34.213 | 1.145 | 1.00 | 124.64 | | O |
| ANISOU | 230 | OE1 | GLU | C | 29 | 16588 | 13223 | 17546 | −3822 | −1636 | −81 | O |
| ATOM | 231 | OE2 | GLU | C | 29 | 75.915 | 34.188 | 3.232 | 1.00 | 130.09 | | O |
| ANISOU | 231 | OE2 | GLU | C | 29 | 17082 | 14152 | 18194 | −3907 | −1843 | −439 | O |
| ATOM | 232 | N | ALA | C | 30 | 77.988 | 28.838 | 1.456 | 1.00 | 79.05 | | N |
| ANISOU | 232 | N | ALA | C | 30 | 10046 | 8694 | 11296 | −3973 | −1111 | 496 | N |
| ATOM | 233 | CA | ALA | C | 30 | 77.964 | 27.588 | 2.211 | 1.00 | 76.20 | | C |
| ANISOU | 233 | CA | ALA | C | 30 | 9602 | 8622 | 10729 | −3891 | −1102 | 458 | C |
| ATOM | 234 | C | ALA | C | 30 | 79.353 | 26.958 | 2.228 | 1.00 | 77.94 | | C |
| ANISOU | 234 | C | ALA | C | 30 | 9627 | 8980 | 11006 | −3991 | −1088 | 588 | C |
| ATOM | 235 | O | ALA | C | 30 | 79.785 | 26.476 | 3.278 | 1.00 | 77.49 | | O |
| ANISOU | 235 | O | ALA | C | 30 | 9481 | 9093 | 10869 | −3997 | −1191 | 491 | O |
| ATOM | 236 | CB | ALA | C | 30 | 76.943 | 26.633 | 1.618 | 1.00 | 74.52 | | C |
| ANISOU | 236 | CB | ALA | C | 30 | 9479 | 8515 | 10322 | −3724 | −956 | 536 | C |
| ATOM | 237 | N | TYR | C | 31 | 80.076 | 27.014 | 1.091 | 1.00 | 73.30 | | N |
| ANISOU | 237 | N | TYR | C | 31 | 8964 | 8339 | 10548 | −4066 | −970 | 797 | N |
| ATOM | 238 | CA | TYR | C | 31 | 81.439 | 26.502 | 1.021 | 1.00 | 73.47 | | C |
| ANISOU | 238 | CA | TYR | C | 31 | 8767 | 8517 | 10630 | −4153 | −957 | 902 | C |
| ATOM | 239 | C | TYR | C | 31 | 82.352 | 27.317 | 1.947 | 1.00 | 79.55 | | C |
| ANISOU | 239 | C | TYR | C | 31 | 9417 | 9260 | 11548 | −4333 | −1125 | 767 | C |
| ATOM | 240 | O | TYR | C | 31 | 83.173 | 26.731 | 2.652 | 1.00 | 79.95 | | O |
| ANISOU | 240 | O | TYR | C | 31 | 9309 | 9509 | 11559 | −4339 | −1203 | 720 | O |
| ATOM | 241 | CB | TYR | C | 31 | 81.980 | 26.498 | −0.422 | 1.00 | 74.65 | | C |
| ANISOU | 241 | CB | TYR | C | 31 | 8843 | 8646 | 10874 | −4209 | −787 | 1135 | C |
| ATOM | 242 | CG | TYR | C | 31 | 83.469 | 26.231 | −0.486 | 1.00 | 77.93 | | C |
| ANISOU | 242 | CG | TYR | C | 31 | 8997 | 9231 | 11380 | −4328 | −785 | 1208 | C |
| ATOM | 243 | CD1 | TYR | C | 31 | 83.971 | 24.939 | −0.368 | 1.00 | 78.91 | | C |
| ANISOU | 243 | CD1 | TYR | C | 31 | 8989 | 9608 | 11386 | −4196 | −765 | 1240 | C |
| ATOM | 244 | CD2 | TYR | C | 31 | 84.381 | 27.278 | −0.568 | 1.00 | 81.57 | | C |
| ANISOU | 244 | CD2 | TYR | C | 31 | 9342 | 9606 | 12045 | −4575 | −824 | 1226 | C |
| ATOM | 245 | CE1 | TYR | C | 31 | 85.343 | 24.691 | −0.376 | 1.00 | 81.63 | | C |
| ANISOU | 245 | CE1 | TYR | C | 31 | 9071 | 10142 | 11804 | −4276 | −780 | 1275 | C |
| ATOM | 246 | CE2 | TYR | C | 31 | 85.756 | 27.045 | −0.552 | 1.00 | 84.41 | | C |
| ANISOU | 246 | CE2 | TYR | C | 31 | 9426 | 10167 | 12478 | −4694 | −827 | 1265 | C |
| ATOM | 247 | CZ | TYR | C | 31 | 86.235 | 25.748 | −0.467 | 1.00 | 91.20 | | C |
| ANISOU | 247 | CZ | TYR | C | 31 | 10137 | 11307 | 13209 | −4528 | −804 | 1282 | C |
| ATOM | 248 | OH | TYR | C | 31 | 87.595 | 25.516 | −0.479 | 1.00 | 94.27 | | O |
| ANISOU | 248 | OH | TYR | C | 31 | 10233 | 11923 | 13662 | −4617 | −814 | 1298 | O |
| ATOM | 249 | N | ARG | C | 32 | 82.206 | 28.659 | 1.936 | 1.00 | 77.13 | | N |
| ANISOU | 249 | N | ARG | C | 32 | 9195 | 8700 | 11411 | −4472 | −1195 | 698 | N |
| ATOM | 250 | CA | ARG | C | 32 | 82.999 | 29.586 | 2.752 | 1.00 | 79.29 | | C |
| ANISOU | 250 | CA | ARG | C | 32 | 9377 | 8897 | 11851 | −4666 | −1366 | 552 | C |
| ATOM | 251 | C | ARG | C | 32 | 82.796 | 29.377 | 4.256 | 1.00 | 82.52 | | C |
| ANISOU | 251 | C | ARG | C | 32 | 9772 | 9443 | 12138 | −4588 | −1541 | 300 | C |
| ATOM | 252 | O | ARG | C | 32 | 83.770 | 29.426 | 5.017 | 1.00 | 84.02 | | O |
| ANISOU | 252 | O | ARG | C | 32 | 9790 | 9751 | 12385 | −4696 | −1658 | 211 | O |
| ATOM | 253 | CB | ARG | C | 32 | 82.736 | 31.049 | 2.346 | 1.00 | 81.48 | | C |
| ANISOU | 253 | CB | ARG | C | 32 | 9808 | 8820 | 12329 | −4809 | −1419 | 532 | C |
| ATOM | 254 | CG | ARG | C | 32 | 83.278 | 31.421 | 0.957 | 1.00 | 94.16 | | C |
| ANISOU | 254 | CG | ARG | C | 32 | 11390 | 10303 | 14083 | −4975 | −1272 | 800 | C |
| ATOM | 255 | CD | ARG | C | 32 | 84.786 | 31.662 | 0.936 | 1.00 | 101.82 | | C |
| ANISOU | 255 | CD | ARG | C | 32 | 12113 | 11355 | 15217 | −5249 | −1290 | 864 | C |
| ATOM | 256 | NE | ARG | C | 32 | 85.114 | 33.091 | 0.891 | 1.00 | 102.15 | | N |
| ANISOU | 256 | NE | ARG | C | 32 | 12232 | 11082 | 15498 | −5514 | −1399 | 839 | N |
| ATOM | 257 | CZ | ARG | C | 32 | 85.557 | 33.801 | 1.924 | 1.00 | 104.57 | | C |
| ANISOU | 257 | CZ | ARG | C | 32 | 12488 | 11312 | 15930 | −5660 | −1599 | 626 | C |
| ATOM | 258 | NH1 | ARG | C | 32 | 85.810 | 35.096 | 1.786 | 1.00 | 82.93 | | N |
| ANISOU | 258 | NH1 | ARG | C | 32 | 9849 | 8244 | 13417 | −5909 | −1702 | 617 | N |
| ATOM | 259 | NH2 | ARG | C | 32 | 85.751 | 33.221 | 3.105 | 1.00 | 89.27 | | N |
| ANISOU | 259 | NH2 | ARG | C | 32 | 10411 | 9618 | 13890 | −5561 | −1708 | 422 | N |
| ATOM | 260 | N | PHE | C | 33 | 81.536 | 29.122 | 4.670 | 1.00 | 76.25 | | N |
| ANISOU | 260 | N | PHE | C | 33 | 9146 | 8666 | 11160 | −4403 | −1553 | 185 | N |
| ATOM | 261 | CA | PHE | C | 33 | 81.151 | 28.861 | 6.060 | 1.00 | 75.48 | | C |
| ANISOU | 261 | CA | PHE | C | 33 | 9053 | 8735 | 10890 | −4316 | −1697 | −44 | C |
| ATOM | 262 | C | PHE | C | 33 | 81.771 | 27.538 | 6.521 | 1.00 | 75.60 | | C |
| ANISOU | 262 | C | PHE | C | 33 | 8932 | 9050 | 10743 | −4256 | −1686 | 32 | C |
| ATOM | 263 | O | PHE | C | 33 | 82.429 | 27.514 | 7.563 | 1.00 | 77.38 | | O |
| ANISOU | 263 | O | PHE | C | 33 | 9040 | 9416 | 10945 | −4300 | −1834 | −97 | O |
| ATOM | 264 | CB | PHE | C | 33 | 79.612 | 28.816 | 6.194 | 1.00 | 76.08 | | C |
| ANISOU | 264 | CB | PHE | C | 33 | 9324 | 8797 | 10786 | −4143 | −1673 | −157 | C |
| ATOM | 265 | CG | PHE | C | 33 | 79.049 | 28.687 | 7.594 | 1.00 | 78.33 | | C |
| ANISOU | 265 | CG | PHE | C | 33 | 9624 | 9270 | 10867 | −4063 | −1809 | −407 | C |
| ATOM | 266 | CD1 | PHE | C | 33 | 78.743 | 29.816 | 8.346 | 1.00 | 84.18 | | C |
| ANISOU | 266 | CD1 | PHE | C | 33 | 10404 | 9909 | 11672 | −4086 | −1980 | −682 | C |
| ATOM | 267 | CD2 | PHE | C | 33 | 78.782 | 27.438 | 8.143 | 1.00 | 79.00 | | C |
| ANISOU | 267 | CD2 | PHE | C | 33 | 9699 | 9633 | 10685 | −3964 | −1769 | −370 | C |
| ATOM | 268 | CE1 | PHE | C | 33 | 78.196 | 29.699 | 9.630 | 1.00 | 85.59 | | C |
| ANISOU | 268 | CE1 | PHE | C | 33 | 10583 | 10305 | 11634 | −4006 | −2097 | −927 | C |
| ATOM | 269 | CE2 | PHE | C | 33 | 78.249 | 27.320 | 9.433 | 1.00 | 82.48 | | C |
| ANISOU | 269 | CE2 | PHE | C | 33 | 10154 | 10278 | 10907 | −3911 | −1884 | −584 | C |
| ATOM | 270 | CZ | PHE | C | 33 | 77.950 | 28.450 | 10.164 | 1.00 | 82.71 | | C |
| ANISOU | 270 | CZ | PHE | C | 33 | 10196 | 10245 | 10986 | −3927 | −2040 | −867 | C |
| ATOM | 271 | N | ALA | C | 34 | 81.571 | 26.453 | 5.730 | 1.00 | 66.12 | | N |
| ANISOU | 271 | N | ALA | C | 34 | 7753 | 7936 | 9432 | −4146 | −1525 | 236 | N |
| ATOM | 272 | CA | ALA | C | 34 | 82.072 | 25.108 | 6.000 | 1.00 | 63.10 | | C |
| ANISOU | 272 | CA | ALA | C | 34 | 7286 | 7793 | 8897 | −4055 | −1515 | 332 | C |
| ATOM | 273 | C | ALA | C | 34 | 83.605 | 25.020 | 6.062 | 1.00 | 66.43 | | C |
| ANISOU | 273 | C | ALA | C | 34 | 7473 | 8319 | 9450 | −4147 | −1575 | 378 | C |
| ATOM | 274 | O | ALA | C | 34 | 84.129 | 24.322 | 6.930 | 1.00 | 65.71 | | O |
| ANISOU | 274 | O | ALA | C | 34 | 7297 | 8426 | 9243 | −4092 | −1686 | 335 | O |
| ATOM | 275 | CB | ALA | C | 34 | 81.535 | 24.147 | 4.965 | 1.00 | 61.14 | | C |
| ANISOU | 275 | CB | ALA | C | 34 | 7123 | 7555 | 8553 | −3929 | −1336 | 525 | C |
| ATOM | 276 | N | GLU | C | 35 | 84.318 | 25.722 | 5.144 | 1.00 | 63.20 | | N |
| ANISOU | 276 | N | GLU | C | 35 | 6954 | 7793 | 9266 | −4291 | −1505 | 467 | N |
| ATOM | 277 | CA | GLU | C | 35 | 85.784 | 25.746 | 5.071 | 1.00 | 64.22 | | C |
| ANISOU | 277 | CA | GLU | C | 35 | 6824 | 8045 | 9532 | −4409 | −1541 | 504 | C |
| ATOM | 278 | C | GLU | C | 35 | 86.388 | 26.302 | 6.348 | 1.00 | 68.86 | | C |
| ANISOU | 278 | C | GLU | C | 35 | 7301 | 8702 | 10161 | −4510 | −1752 | 292 | C |
| ATOM | 279 | O | GLU | C | 35 | 87.313 | 25.700 | 6.890 | 1.00 | 69.10 | | O |
| ANISOU | 279 | O | GLU | C | 35 | 7149 | 8956 | 10148 | −4478 | −1841 | 266 | O |
| ATOM | 280 | CB | GLU | C | 35 | 86.262 | 26.532 | 3.842 | 1.00 | 66.66 | | C |
| ANISOU | 280 | CB | GLU | C | 35 | 7060 | 8209 | 10057 | −4589 | −1414 | 639 | C |
| ATOM | 281 | CG | GLU | C | 35 | 87.746 | 26.375 | 3.534 | 1.00 | 80.81 | | C |
| ANISOU | 281 | CG | GLU | C | 35 | 8553 | 10189 | 11963 | −4709 | −1402 | 704 | C |
| ATOM | 282 | CD | GLU | C | 35 | 88.728 | 27.122 | 4.423 | 1.00 | 96.72 | | C |
| ANISOU | 282 | CD | GLU | C | 35 | 10383 | 12254 | 14113 | −4905 | −1576 | 537 | C |
| ATOM | 283 | OE1 | GLU | C | 35 | 88.484 | 28.316 | 4.712 | 1.00 | 89.55 | | O |
| ANISOU | 283 | OE1 | GLU | C | 35 | 9567 | 11119 | 13341 | −5079 | −1655 | 432 | O |
| ATOM | 284 | OE2 | GLU | C | 35 | 89.736 | 26.507 | 4.841 | 1.00 | 81.83 | | O |
| ANISOU | 284 | OE2 | GLU | C | 35 | 8265 | 10634 | 12194 | −4872 | −1649 | 496 | O |
| ATOM | 285 | N | GLU | C | 36 | 85.869 | 27.452 | 6.812 | 1.00 | 65.89 | | N |
| ANISOU | 285 | N | GLU | C | 36 | 7034 | 8134 | 9866 | −4614 | −1844 | 127 | N |
| ATOM | 286 | CA | GLU | C | 36 | 86.277 | 28.124 | 8.042 | 1.00 | 67.82 | | C |
| ANISOU | 286 | CA | GLU | C | 36 | 7203 | 8414 | 10153 | −4709 | −2056 | −115 | C |
| ATOM | 287 | C | GLU | C | 36 | 85.918 | 27.217 | 9.226 | 1.00 | 70.48 | | C |
| ANISOU | 287 | C | GLU | C | 36 | 7577 | 8983 | 10217 | −4524 | −2163 | −221 | C |
| ATOM | 288 | O | GLU | C | 36 | 86.737 | 27.019 | 10.114 | 1.00 | 70.39 | | O |
| ANISOU | 288 | O | GLU | C | 36 | 7412 | 9163 | 10170 | −4537 | −2313 | −330 | O |
| ATOM | 289 | CB | GLU | C | 36 | 85.548 | 29.472 | 8.143 | 1.00 | 70.35 | | C |
| ANISOU | 289 | CB | GLU | C | 36 | 7686 | 8441 | 10602 | −4810 | −2120 | −267 | C |
| ATOM | 290 | CG | GLU | C | 36 | 86.129 | 30.452 | 9.151 | 1.00 | 87.51 | | C |
| ANISOU | 290 | CG | GLU | C | 36 | 9771 | 10584 | 12896 | −4959 | −2339 | −524 | C |
| ATOM | 291 | CD | GLU | C | 36 | 85.420 | 31.793 | 9.246 | 1.00 | 119.88 | | C |
| ANISOU | 291 | CD | GLU | C | 36 | 14049 | 14362 | 17138 | −5038 | −2430 | −695 | C |
| ATOM | 292 | OE1 | GLU | C | 36 | 84.205 | 31.856 | 8.945 | 1.00 | 130.72 | | O |
| ANISOU | 292 | OE1 | GLU | C | 36 | 15638 | 15603 | 18426 | −4898 | −2363 | −690 | O |
| ATOM | 293 | OE2 | GLU | C | 36 | 86.078 | 32.781 | 9.644 | 1.00 | 112.89 | | O |
| ANISOU | 293 | OE2 | GLU | C | 36 | 13087 | 13359 | 16448 | −5232 | −2582 | −851 | O |
| ATOM | 294 | N | ALA | C | 37 | 84.710 | 26.614 | 9.185 | 1.00 | 66.51 | | N |
| ANISOU | 294 | N | ALA | C | 37 | 7278 | 8480 | 9513 | −4359 | −2081 | −176 | N |
| ATOM | 295 | CA | ALA | C | 37 | 84.165 | 25.694 | 10.192 | 1.00 | 66.04 | | C |
| ANISOU | 295 | CA | ALA | C | 37 | 7301 | 8630 | 9163 | −4204 | −2151 | −233 | C |
| ATOM | 296 | C | ALA | C | 37 | 85.024 | 24.438 | 10.408 | 1.00 | 70.98 | | C |
| ANISOU | 296 | C | ALA | C | 37 | 7812 | 9487 | 9671 | −4108 | −2183 | −107 | C |
| ATOM | 297 | O | ALA | C | 37 | 85.051 | 23.910 | 11.516 | 1.00 | 70.99 | | O |
| ANISOU | 297 | O | ALA | C | 37 | 7826 | 9677 | 9468 | −4033 | −2315 | −184 | O |
| ATOM | 298 | CB | ALA | C | 37 | 82.744 | 25.293 | 9.815 | 1.00 | 64.38 | | C |
| ANISOU | 298 | CB | ALA | C | 37 | 7311 | 8362 | 8787 | −4087 | −2019 | −173 | C |
| ATOM | 299 | N | HIS | C | 38 | 85.690 | 23.950 | 9.344 | 1.00 | 68.15 | | N |
| ANISOU | 299 | N | HIS | C | 38 | 7350 | 9119 | 9425 | −4097 | −2068 | 83 | N |
| ATOM | 300 | CA | HIS | C | 38 | 86.569 | 22.783 | 9.382 | 1.00 | 68.45 | | C |
| ANISOU | 300 | CA | HIS | C | 38 | 7270 | 9356 | 9382 | −3976 | −2105 | 193 | C |
| ATOM | 301 | C | HIS | C | 38 | 88.016 | 23.204 | 9.166 | 1.00 | 76.46 | | C |
| ANISOU | 301 | C | HIS | C | 38 | 7995 | 10453 | 10604 | −4087 | −2155 | 167 | C |
| ATOM | 302 | O | HIS | C | 38 | 88.831 | 22.362 | 8.780 | 1.00 | 77.00 | | O |
| ANISOU | 302 | O | HIS | C | 38 | 7926 | 10666 | 10664 | −3987 | −2142 | 272 | O |
| ATOM | 303 | CB | HIS | C | 38 | 86.157 | 21.757 | 8.316 | 1.00 | 66.80 | | C |
| ANISOU | 303 | CB | HIS | C | 38 | 7165 | 9111 | 9106 | −3838 | −1934 | 410 | C |
| ATOM | 304 | CG | HIS | C | 38 | 84.834 | 21.114 | 8.573 | 1.00 | 68.11 | | C |
| ANISOU | 304 | CG | HIS | C | 38 | 7591 | 9246 | 9042 | −3732 | −1893 | 447 | C |
| ATOM | 305 | CD2 | HIS | C | 38 | 84.388 | 20.454 | 9.667 | 1.00 | 69.75 | | C |
| ANISOU | 305 | CD2 | HIS | C | 38 | 7921 | 9573 | 9007 | −3660 | −2008 | 400 | C |
| ATOM | 306 | ND1 | HIS | C | 38 | 83.834 | 21.128 | 7.624 | 1.00 | 67.86 | | N |
| ANISOU | 306 | ND1 | HIS | C | 38 | 7708 | 9066 | 9011 | −3711 | −1713 | 544 | N |
| ATOM | 307 | CE1 | HIS | C | 38 | 82.814 | 20.478 | 8.164 | 1.00 | 65.99 | | C |
| ANISOU | 307 | CE1 | HIS | C | 38 | 7664 | 8867 | 8540 | −3637 | −1721 | 543 | C |
| ATOM | 308 | NE2 | HIS | C | 38 | 83.102 | 20.053 | 9.391 | 1.00 | 67.48 | | N |
| ANISOU | 308 | NE2 | HIS | C | 38 | 7851 | 9217 | 8573 | −3614 | −1891 | 468 | N |
| ATOM | 309 | N | ARG | C | 39 | 88.334 | 24.509 | 9.391 | 1.00 | 75.53 | | N |
| ANISOU | 309 | N | ARG | C | 39 | 7781 | 10244 | 10674 | −4296 | −2217 | 18 | N |
| ATOM | 310 | CA | ARG | C | 39 | 89.684 | 25.066 | 9.222 | 1.00 | 78.51 | | C |
| ANISOU | 310 | CA | ARG | C | 39 | 7870 | 10699 | 11261 | −4463 | −2264 | −25 | C |
| ATOM | 311 | C | ARG | C | 39 | 90.597 | 24.445 | 10.284 | 1.00 | 87.75 | | C |
| ANISOU | 311 | C | ARG | C | 39 | 8862 | 12156 | 12323 | −4369 | −2459 | −132 | C |
| ATOM | 312 | O | ARG | C | 39 | 90.435 | 24.725 | 11.474 | 1.00 | 90.40 | | O |
| ANISOU | 312 | O | ARG | C | 39 | 9224 | 12545 | 12577 | −4378 | −2634 | −316 | O |
| ATOM | 313 | CB | ARG | C | 39 | 89.672 | 26.615 | 9.299 | 1.00 | 77.35 | | C |
| ANISOU | 313 | CB | ARG | C | 39 | 7714 | 10342 | 11333 | −4724 | −2306 | −166 | C |
| ATOM | 314 | CG | ARG | C | 39 | 90.841 | 27.319 | 8.607 | 1.00 | 79.05 | | C |
| ANISOU | 314 | CG | ARG | C | 39 | 7674 | 10557 | 11804 | −4970 | −2269 | −135 | C |
| ATOM | 315 | CD | ARG | C | 39 | 90.364 | 28.334 | 7.573 | 1.00 | 77.43 | | C |
| ANISOU | 315 | CD | ARG | C | 39 | 7590 | 10038 | 11790 | −5163 | −2129 | −37 | C |
| ATOM | 316 | NE | ARG | C | 39 | 90.028 | 29.633 | 8.161 | 1.00 | 82.14 | | N |
| ANISOU | 316 | NE | ARG | C | 39 | 8305 | 10391 | 12516 | −5328 | −2260 | −227 | N |
| ATOM | 317 | CZ | ARG | C | 39 | 89.448 | 30.638 | 7.505 | 1.00 | 94.04 | | C |
| ANISOU | 317 | CZ | ARG | C | 39 | 9983 | 11569 | 14179 | −5472 | −2198 | −182 | C |
| ATOM | 318 | NH1 | ARG | C | 39 | 89.114 | 30.503 | 6.229 | 1.00 | 79.20 | | N |
| ANISOU | 318 | NH1 | ARG | C | 39 | 8176 | 9582 | 12335 | −5481 | −1994 | 58 | N |
| ATOM | 319 | NH2 | ARG | C | 39 | 89.195 | 31.785 | 8.122 | 1.00 | 80.91 | | N |
| ANISOU | 319 | NH2 | ARG | C | 39 | 8429 | 9679 | 12633 | −5593 | −2353 | −386 | N |
| ATOM | 320 | N | GLY | C | 40 | 91.485 | 23.553 | 9.843 | 1.00 | 84.60 | | N |
| ANISOU | 320 | N | GLY | C | 40 | 8295 | 11947 | 11900 | −4248 | −2435 | −21 | N |
| ATOM | 321 | CA | GLY | C | 40 | 92.411 | 22.836 | 10.709 | 1.00 | 86.09 | | C |
| ANISOU | 321 | CA | GLY | C | 40 | 8312 | 12419 | 11981 | −4111 | −2624 | −100 | C |
| ATOM | 322 | C | GLY | C | 40 | 91.915 | 21.467 | 11.125 | 1.00 | 88.48 | | C |
| ANISOU | 322 | C | GLY | C | 40 | 8810 | 12796 | 12012 | −3827 | −2681 | −7 | C |
| ATOM | 323 | O | GLY | C | 40 | 92.658 | 20.712 | 11.754 | 1.00 | 89.21 | | O |
| ANISOU | 323 | O | GLY | C | 40 | 8798 | 13106 | 11990 | −3669 | −2849 | −43 | O |
| ATOM | 324 | N | GLN | C | 41 | 90.647 | 21.145 | 10.800 | 1.00 | 83.58 | | N |
| ANISOU | 324 | N | GLN | C | 41 | 8483 | 11994 | 11281 | −3765 | −2556 | 111 | N |
| ATOM | 325 | CA | GLN | C | 41 | 90.045 | 19.841 | 11.095 | 1.00 | 82.95 | | C |
| ANISOU | 325 | CA | GLN | C | 41 | 8628 | 11944 | 10945 | −3537 | −2589 | 229 | C |
| ATOM | 326 | C | GLN | C | 41 | 90.554 | 18.845 | 10.057 | 1.00 | 89.74 | | C |
| ANISOU | 326 | C | GLN | C | 41 | 9424 | 12844 | 11829 | −3368 | −2506 | 391 | C |
| ATOM | 327 | O | GLN | C | 41 | 90.547 | 19.148 | 8.856 | 1.00 | 89.24 | | O |
| ANISOU | 327 | O | GLN | C | 41 | 9288 | 12691 | 11926 | −3436 | −2321 | 467 | O |
| ATOM | 328 | CB | GLN | C | 41 | 88.511 | 19.920 | 11.077 | 1.00 | 81.59 | | C |
| ANISOU | 328 | CB | GLN | C | 41 | 8759 | 11586 | 10657 | −3565 | −2475 | 276 | C |
| ATOM | 329 | CG | GLN | C | 41 | 87.826 | 18.687 | 11.662 | 1.00 | 93.64 | | C |
| ANISOU | 329 | CG | GLN | C | 41 | 10531 | 13155 | 11894 | −3395 | −2535 | 375 | C |
| ATOM | 330 | CD | GLN | C | 41 | 86.355 | 18.905 | 11.932 | 1.00 | 114.40 | | C |
| ANISOU | 330 | CD | GLN | C | 41 | 13407 | 15679 | 14381 | −3462 | −2456 | 359 | C |
| ATOM | 331 | NE2 | GLN | C | 41 | 85.545 | 17.878 | 11.697 | 1.00 | 104.10 | | N |
| ANISOU | 331 | NE2 | GLN | C | 41 | 12327 | 14324 | 12901 | −3364 | −2383 | 514 | N |
| ATOM | 332 | OE1 | GLN | C | 41 | 85.928 | 19.975 | 12.386 | 1.00 | 111.14 | | O |
| ANISOU | 332 | OE1 | GLN | C | 41 | 12983 | 15239 | 14007 | −3597 | −2472 | 194 | O |
| ATOM | 333 | N | LEU | C | 42 | 91.031 | 17.681 | 10.514 | 1.00 | 88.89 | | N |
| ANISOU | 333 | N | LEU | C | 42 | 9342 | 12873 | 11561 | −3141 | −2654 | 436 | N |
| ATOM | 334 | CA | LEU | C | 42 | 91.585 | 16.676 | 9.611 | 1.00 | 90.04 | | C |
| ANISOU | 334 | CA | LEU | C | 42 | 9420 | 13066 | 11724 | −2939 | −2613 | 554 | C |
| ATOM | 335 | C | LEU | C | 42 | 90.745 | 15.397 | 9.529 | 1.00 | 96.26 | | C |
| ANISOU | 335 | C | LEU | C | 42 | 10526 | 13733 | 12314 | −2742 | −2607 | 716 | C |
| ATOM | 336 | O | LEU | C | 42 | 90.134 | 14.992 | 10.518 | 1.00 | 95.63 | | O |
| ANISOU | 336 | O | LEU | C | 42 | 10680 | 13626 | 12031 | −2713 | −2721 | 736 | O |
| ATOM | 337 | CB | LEU | C | 42 | 93.048 | 16.366 | 9.981 | 1.00 | 92.81 | | C |
| ANISOU | 337 | CB | LEU | C | 42 | 9482 | 13680 | 12101 | −2809 | −2801 | 458 | C |
| ATOM | 338 | CG | LEU | C | 42 | 93.956 | 15.908 | 8.839 | 1.00 | 98.01 | | C |
| ANISOU | 338 | CG | LEU | C | 42 | 9898 | 14458 | 12885 | −2687 | −2721 | 492 | C |
| ATOM | 339 | CD1 | LEU | C | 42 | 94.643 | 17.087 | 8.170 | 1.00 | 99.06 | | C |
| ANISOU | 339 | CD1 | LEU | C | 42 | 9703 | 14680 | 13255 | −2940 | −2591 | 408 | C |
| ATOM | 340 | CD2 | LEU | C | 42 | 94.988 | 14.928 | 9.336 | 1.00 | 103.20 | | C |
| ANISOU | 340 | CD2 | LEU | C | 42 | 10428 | 15336 | 13448 | −2403 | −2951 | 442 | C |
| ATOM | 341 | N | ARG | C | 43 | 90.713 | 14.772 | 8.337 | 1.00 | 94.90 | | N |
| ANISOU | 341 | N | ARG | C | 43 | 10361 | 13499 | 12197 | −2622 | −2474 | 828 | N |
| ATOM | 342 | CA | ARG | C | 43 | 89.987 | 13.527 | 8.095 | 1.00 | 94.66 | | C |
| ANISOU | 342 | CA | ARG | C | 43 | 10619 | 13334 | 12012 | −2440 | −2467 | 975 | C |
| ATOM | 343 | C | ARG | C | 43 | 90.761 | 12.342 | 8.676 | 1.00 | 103.43 | | C |
| ANISOU | 343 | C | ARG | C | 43 | 11753 | 14547 | 12998 | −2169 | −2709 | 993 | C |
| ATOM | 344 | O | ARG | C | 43 | 92.001 | 12.371 | 8.722 | 1.00 | 104.85 | | O |
| ANISOU | 344 | O | ARG | C | 43 | 11652 | 14927 | 13262 | −2066 | −2821 | 896 | O |
| ATOM | 345 | CB | ARG | C | 43 | 89.717 | 13.324 | 6.590 | 1.00 | 93.12 | | C |
| ANISOU | 345 | CB | ARG | C | 43 | 10411 | 13040 | 11929 | −2405 | −2245 | 1063 | C |
| ATOM | 346 | N | ARG | C | 44 | 90.020 | 11.307 | 9.131 | 1.00 | 101.91 | | N |
| ANISOU | 346 | N | ARG | C | 44 | 11900 | 14220 | 12601 | −2059 | −2797 | 1116 | N |
| ATOM | 347 | CA | ARG | C | 44 | 90.586 | 10.078 | 9.698 | 1.00 | 104.77 | | C |
| ANISOU | 347 | CA | ARG | C | 44 | 12375 | 14611 | 12821 | −1789 | −3049 | 1168 | C |
| ATOM | 348 | C | ARG | C | 44 | 91.524 | 9.420 | 8.673 | 1.00 | 111.16 | | C |
| ANISOU | 348 | C | ARG | C | 44 | 13005 | 15475 | 13757 | −1534 | −3059 | 1150 | C |
| ATOM | 349 | O | ARG | C | 44 | 92.709 | 9.211 | 8.955 | 1.00 | 112.74 | | O |
| ANISOU | 349 | O | ARG | C | 44 | 12984 | 15867 | 13984 | −1353 | −3239 | 1053 | O |
| ATOM | 350 | CB | ARG | C | 44 | 89.457 | 9.113 | 10.105 | 1.00 | 104.47 | | C |
| ANISOU | 350 | CB | ARG | C | 44 | 12768 | 14365 | 12561 | −1771 | −3090 | 1338 | C |
| ATOM | 351 | N | SER | C | 45 | 90.989 | 9.179 | 7.457 | 1.00 | 107.13 | | N |
| ANISOU | 351 | N | SER | C | 45 | 12555 | 14825 | 13323 | −1524 | −2857 | 1219 | N |
| ATOM | 352 | CA | SER | C | 45 | 91.687 | 8.584 | 6.322 | 1.00 | 107.71 | | C |
| ANISOU | 352 | CA | SER | C | 45 | 12466 | 14953 | 13507 | −1293 | −2823 | 1193 | C |
| ATOM | 353 | C | SER | C | 45 | 92.750 | 9.543 | 5.759 | 1.00 | 111.13 | | C |
| ANISOU | 353 | C | SER | C | 45 | 12444 | 15649 | 14131 | −1363 | −2723 | 1052 | C |
| ATOM | 354 | O | SER | C | 45 | 93.895 | 9.540 | 6.228 | 1.00 | 112.59 | | O |
| ANISOU | 354 | O | SER | C | 45 | 12382 | 16061 | 14336 | −1242 | −2895 | 936 | O |
| ATOM | 355 | CB | SER | C | 45 | 90.689 | 8.161 | 5.236 | 1.00 | 109.48 | | C |
| ANISOU | 355 | CB | SER | C | 45 | 12892 | 14963 | 13740 | −1298 | −2625 | 1299 | C |
| ATOM | 356 | OG | SER | C | 45 | 89.871 | 7.069 | 5.630 | 1.00 | 117.77 | | O |
| ANISOU | 356 | OG | SER | C | 45 | 14342 | 15782 | 14622 | −1205 | −2739 | 1426 | O |
| ATOM | 357 | N | GLY | C | 46 | 92.326 | 10.380 | 4.809 | 1.00 | 105.20 | | N |
| ANISOU | 357 | N | GLY | C | 46 | 11596 | 14870 | 13506 | −1574 | −2454 | 1067 | N |
| ATOM | 358 | CA | GLY | C | 46 | 93.158 | 11.336 | 4.086 | 1.00 | 105.18 | | C |
| ANISOU | 358 | CA | GLY | C | 46 | 11200 | 15082 | 13682 | −1705 | −2310 | 976 | C |
| ATOM | 359 | C | GLY | C | 46 | 93.690 | 12.556 | 4.818 | 1.00 | 106.98 | | C |
| ANISOU | 359 | C | GLY | C | 46 | 11202 | 15452 | 13993 | −1950 | −2346 | 870 | C |
| ATOM | 360 | O | GLY | C | 46 | 94.209 | 12.462 | 5.945 | 1.00 | 107.43 | | O |
| ANISOU | 360 | O | GLY | C | 46 | 11223 | 15612 | 13981 | −1890 | −2572 | 790 | O |
| ATOM | 361 | N | GLU | C | 47 | 93.600 | 13.714 | 4.126 | 1.00 | 99.99 | | N |
| ANISOU | 361 | N | GLU | C | 47 | 10163 | 14572 | 13259 | −2227 | −2130 | 869 | N |
| ATOM | 362 | CA | GLU | C | 47 | 94.145 | 14.968 | 4.621 | 1.00 | 99.68 | | C |
| ANISOU | 362 | CA | GLU | C | 47 | 9894 | 14645 | 13334 | −2492 | −2148 | 762 | C |
| ATOM | 363 | C | GLU | C | 47 | 93.028 | 15.959 | 5.085 | 1.00 | 96.04 | | C |
| ANISOU | 363 | C | GLU | C | 47 | 9656 | 13945 | 12890 | −2767 | −2069 | 789 | C |
| ATOM | 364 | O | GLU | C | 47 | 92.248 | 15.520 | 5.930 | 1.00 | 93.97 | | O |
| ANISOU | 364 | O | GLU | C | 47 | 9677 | 13550 | 12476 | −2704 | −2174 | 811 | O |
| ATOM | 365 | CB | GLU | C | 47 | 95.137 | 15.551 | 3.600 | 1.00 | 103.08 | | C |
| ANISOU | 365 | CB | GLU | C | 47 | 9922 | 15312 | 13931 | −2598 | −2016 | 715 | C |
| ATOM | 366 | N | PRO | C | 48 | 92.936 | 17.262 | 4.665 | 1.00 | 88.58 | | N |
| ANISOU | 366 | N | PRO | C | 48 | 8603 | 12945 | 12107 | −3063 | −1914 | 779 | N |
| ATOM | 367 | CA | PRO | C | 48 | 91.920 | 18.161 | 5.243 | 1.00 | 85.91 | | C |
| ANISOU | 367 | CA | PRO | C | 48 | 8480 | 12384 | 11776 | −3272 | −1893 | 763 | C |
| ATOM | 368 | C | PRO | C | 48 | 90.507 | 17.627 | 5.304 | 1.00 | 85.18 | | C |
| ANISOU | 368 | C | PRO | C | 48 | 8760 | 12074 | 11530 | −3182 | −1846 | 855 | C |
| ATOM | 369 | O | PRO | C | 48 | 90.129 | 16.822 | 4.457 | 1.00 | 83.38 | | O |
| ANISOU | 369 | O | PRO | C | 48 | 8639 | 11795 | 11246 | −3029 | −1743 | 970 | O |
| ATOM | 370 | CB | PRO | C | 48 | 92.002 | 19.411 | 4.363 | 1.00 | 87.79 | | C |
| ANISOU | 370 | CB | PRO | C | 48 | 8585 | 12556 | 12215 | −3545 | −1708 | 787 | C |
| ATOM | 371 | CG | PRO | C | 48 | 92.829 | 19.014 | 3.196 | 1.00 | 93.35 | | C |
| ANISOU | 371 | CG | PRO | C | 48 | 9043 | 13445 | 12978 | −3473 | −1591 | 853 | C |
| ATOM | 372 | CD | PRO | C | 48 | 93.771 | 18.009 | 3.709 | 1.00 | 90.78 | | C |
| ANISOU | 372 | CD | PRO | C | 48 | 8562 | 13366 | 12564 | −3230 | −1771 | 774 | C |
| ATOM | 373 | N | TYR | C | 49 | 89.734 | 18.070 | 6.315 | 1.00 | 79.81 | | N |
| ANISOU | 373 | N | TYR | C | 49 | 8262 | 11288 | 10773 | −3278 | −1926 | 788 | N |
| ATOM | 374 | CA | TYR | C | 49 | 88.358 | 17.609 | 6.485 | 1.00 | 76.56 | | C |
| ANISOU | 374 | CA | TYR | C | 49 | 8181 | 10714 | 10195 | −3223 | −1885 | 856 | C |
| ATOM | 375 | C | TYR | C | 49 | 87.403 | 18.179 | 5.424 | 1.00 | 76.85 | | C |
| ANISOU | 375 | C | TYR | C | 49 | 8329 | 10560 | 10309 | −3324 | −1662 | 928 | C |
| ATOM | 376 | O | TYR | C | 49 | 86.602 | 17.422 | 4.874 | 1.00 | 74.55 | | O |
| ANISOU | 376 | O | TYR | C | 49 | 8226 | 10182 | 9917 | −3214 | −1566 | 1033 | O |
| ATOM | 377 | CB | TYR | C | 49 | 87.831 | 17.841 | 7.916 | 1.00 | 77.67 | | C |
| ANISOU | 377 | CB | TYR | C | 49 | 8460 | 10861 | 10191 | −3277 | −2041 | 748 | C |
| ATOM | 378 | CG | TYR | C | 49 | 86.596 | 17.023 | 8.204 | 1.00 | 77.15 | | C |
| ANISOU | 378 | CG | TYR | C | 49 | 8705 | 10708 | 9900 | −3193 | −2025 | 829 | C |
| ATOM | 379 | CD1 | TYR | C | 49 | 86.693 | 15.683 | 8.555 | 1.00 | 79.34 | | C |
| ANISOU | 379 | CD1 | TYR | C | 49 | 9107 | 11040 | 9998 | −3007 | −2134 | 922 | C |
| ATOM | 380 | CD2 | TYR | C | 49 | 85.324 | 17.559 | 8.026 | 1.00 | 76.33 | | C |
| ANISOU | 380 | CD2 | TYR | C | 49 | 8773 | 10463 | 9765 | −3298 | −1896 | 820 | C |
| ATOM | 381 | CE1 | TYR | C | 49 | 85.555 | 14.901 | 8.748 | 1.00 | 79.39 | | C |
| ANISOU | 381 | CE1 | TYR | C | 49 | 9405 | 10960 | 9800 | −2969 | −2109 | 1017 | C |
| ATOM | 382 | CE2 | TYR | C | 49 | 84.179 | 16.786 | 8.205 | 1.00 | 75.91 | | C |
| ANISOU | 382 | CE2 | TYR | C | 49 | 8980 | 10361 | 9503 | −3245 | −1862 | 893 | C |
| ATOM | 383 | CZ | TYR | C | 49 | 84.298 | 15.457 | 8.575 | 1.00 | 84.06 | | C |
| ANISOU | 383 | CZ | TYR | C | 49 | 10135 | 11446 | 10358 | −3099 | −1964 | 998 | C |
| ATOM | 384 | OH | TYR | C | 49 | 83.171 | 14.691 | 8.763 | 1.00 | 83.25 | | O |
| ANISOU | 384 | OH | TYR | C | 49 | 10293 | 11293 | 10046 | −3087 | −1931 | 1082 | O |
| ATOM | 385 | N | ILE | C | 50 | 87.517 | 19.488 | 5.110 | 1.00 | 72.39 | | N |
| ANISOU | 385 | N | ILE | C | 50 | 7653 | 9926 | 9926 | −3532 | −1590 | 874 | N |
| ATOM | 386 | CA | ILE | C | 50 | 86.683 | 20.208 | 4.131 | 1.00 | 69.69 | | C |
| ANISOU | 386 | CA | ILE | C | 50 | 7413 | 9395 | 9672 | −3637 | −1402 | 938 | C |
| ATOM | 387 | C | ILE | C | 50 | 86.490 | 19.426 | 2.824 | 1.00 | 72.03 | | C |
| ANISOU | 387 | C | ILE | C | 50 | 7743 | 9679 | 9945 | −3504 | −1232 | 1097 | C |
| ATOM | 388 | O | ILE | C | 50 | 85.434 | 19.549 | 2.201 | 1.00 | 69.97 | | O |
| ANISOU | 388 | O | ILE | C | 50 | 7663 | 9270 | 9654 | −3501 | −1102 | 1156 | O |
| ATOM | 389 | CB | ILE | C | 50 | 87.204 | 21.664 | 3.887 | 1.00 | 73.66 | | C |
| ANISOU | 389 | CB | ILE | C | 50 | 7757 | 9832 | 10400 | −3882 | −1377 | 884 | C |
| ATOM | 390 | CG1 | ILE | C | 50 | 86.213 | 22.532 | 3.068 | 1.00 | 72.22 | | C |
| ANISOU | 390 | CG1 | ILE | C | 50 | 7733 | 9413 | 10297 | −3985 | −1225 | 942 | C |
| ATOM | 391 | CG2 | ILE | C | 50 | 88.604 | 21.695 | 3.274 | 1.00 | 75.55 | | C |
| ANISOU | 391 | CG2 | ILE | C | 50 | 7688 | 10244 | 10774 | −3937 | −1351 | 925 | C |
| ATOM | 392 | CD1 | ILE | C | 50 | 84.781 | 22.593 | 3.583 | 1.00 | 74.75 | | C |
| ANISOU | 392 | CD1 | ILE | C | 50 | 8326 | 9589 | 10488 | −3931 | −1240 | 874 | C |
| ATOM | 393 | N | THR | C | 51 | 87.493 | 18.598 | 2.443 | 1.00 | 69.00 | | N |
| ANISOU | 393 | N | THR | C | 51 | 7184 | 9468 | 9566 | −3374 | −1246 | 1143 | N |
| ATOM | 394 | CA | THR | C | 51 | 87.479 | 17.746 | 1.250 | 1.00 | 67.32 | | C |
| ANISOU | 394 | CA | THR | C | 51 | 6969 | 9285 | 9323 | −3213 | −1112 | 1262 | C |
| ATOM | 395 | C | THR | C | 51 | 86.238 | 16.871 | 1.248 | 1.00 | 68.41 | | C |
| ANISOU | 395 | C | THR | C | 51 | 7406 | 9293 | 9292 | −3073 | −1087 | 1316 | C |
| ATOM | 396 | O | THR | C | 51 | 85.600 | 16.775 | 0.205 | 1.00 | 66.99 | | O |
| ANISOU | 396 | O | THR | C | 51 | 7313 | 9031 | 9111 | −3042 | −925 | 1400 | O |
| ATOM | 397 | CB | THR | C | 51 | 88.731 | 16.873 | 1.174 | 1.00 | 74.52 | | C |
| ANISOU | 397 | CB | THR | C | 51 | 7659 | 10424 | 10232 | −3046 | −1198 | 1248 | C |
| ATOM | 398 | CG2 | THR | C | 51 | 90.020 | 17.679 | 1.145 | 1.00 | 74.69 | | C |
| ANISOU | 398 | CG2 | THR | C | 51 | 7342 | 10628 | 10410 | −3198 | −1218 | 1182 | C |
| ATOM | 399 | OG1 | THR | C | 51 | 88.723 | 15.975 | 2.278 | 1.00 | 75.09 | | O |
| ANISOU | 399 | OG1 | THR | C | 51 | 7851 | 10521 | 10159 | −2889 | −1397 | 1201 | O |
| ATOM | 400 | N | HIS | C | 52 | 85.883 | 16.270 | 2.426 | 1.00 | 63.68 | | N |
| ANISOU | 400 | N | HIS | C | 52 | 6964 | 8688 | 8543 | −3007 | −1248 | 1271 | N |
| ATOM | 401 | CA | HIS | C | 52 | 84.699 | 15.431 | 2.610 | 1.00 | 61.02 | | C |
| ANISOU | 401 | CA | HIS | C | 52 | 6914 | 8244 | 8027 | −2921 | −1243 | 1320 | C |
| ATOM | 402 | C | HIS | C | 52 | 83.378 | 16.185 | 2.290 | 1.00 | 65.81 | | C |
| ANISOU | 402 | C | HIS | C | 52 | 7677 | 8704 | 8624 | −3046 | −1099 | 1317 | C |
| ATOM | 403 | O | HIS | C | 52 | 82.746 | 15.801 | 1.292 | 1.00 | 64.06 | | O |
| ANISOU | 403 | O | HIS | C | 52 | 7542 | 8411 | 8385 | −2982 | −957 | 1394 | O |
| ATOM | 404 | CB | HIS | C | 52 | 84.671 | 14.759 | 3.993 | 1.00 | 61.48 | | C |
| ANISOU | 404 | CB | HIS | C | 52 | 7096 | 8348 | 7916 | −2872 | −1446 | 1285 | C |
| ATOM | 405 | CG | HIS | C | 52 | 83.342 | 14.164 | 4.369 | 1.00 | 62.92 | | C |
| ANISOU | 405 | CG | HIS | C | 52 | 7572 | 8434 | 7903 | −2874 | −1435 | 1327 | C |
| ATOM | 406 | CD2 | HIS | C | 52 | 82.421 | 14.592 | 5.266 | 1.00 | 64.10 | | C |
| ANISOU | 406 | CD2 | HIS | C | 52 | 7857 | 8571 | 7926 | −2999 | −1463 | 1265 | C |
| ATOM | 407 | ND1 | HIS | C | 52 | 82.891 | 12.987 | 3.807 | 1.00 | 63.52 | | N |
| ANISOU | 407 | ND1 | HIS | C | 52 | 7812 | 8435 | 7886 | −2744 | −1396 | 1433 | N |
| ATOM | 408 | CE1 | HIS | C | 52 | 81.716 | 12.743 | 4.361 | 1.00 | 61.92 | | C |
| ANISOU | 408 | CE1 | HIS | C | 52 | 7838 | 8179 | 7510 | −2820 | −1393 | 1444 | C |
| ATOM | 409 | NE2 | HIS | C | 52 | 81.391 | 13.678 | 5.248 | 1.00 | 62.53 | | N |
| ANISOU | 409 | NE2 | HIS | C | 52 | 7897 | 8310 | 7550 | −2967 | −1428 | 1343 | N |
| ATOM | 410 | N | PRO | C | 53 | 82.934 | 17.252 | 3.034 | 1.00 | 63.45 | | N |
| ANISOU | 410 | N | PRO | C | 53 | 7408 | 8364 | 8338 | −3203 | −1134 | 1213 | N |
| ATOM | 411 | CA | PRO | C | 53 | 81.662 | 17.899 | 2.670 | 1.00 | 61.91 | | C |
| ANISOU | 411 | CA | PRO | C | 53 | 7358 | 8039 | 8124 | −3275 | −1012 | 1192 | C |
| ATOM | 412 | C | PRO | C | 53 | 81.615 | 18.411 | 1.225 | 1.00 | 66.78 | | C |
| ANISOU | 412 | C | PRO | C | 53 | 7921 | 8569 | 8885 | −3289 | −833 | 1272 | C |
| ATOM | 413 | O | PRO | C | 53 | 80.565 | 18.308 | 0.595 | 1.00 | 66.33 | | O |
| ANISOU | 413 | O | PRO | C | 53 | 8007 | 8432 | 8765 | −3250 | −717 | 1307 | O |
| ATOM | 414 | CB | PRO | C | 53 | 81.510 | 18.997 | 3.722 | 1.00 | 64.38 | | C |
| ANISOU | 414 | CB | PRO | C | 53 | 7662 | 8342 | 8458 | −3414 | −1116 | 1038 | C |
| ATOM | 415 | CG | PRO | C | 53 | 82.308 | 18.525 | 4.882 | 1.00 | 70.17 | | C |
| ANISOU | 415 | CG | PRO | C | 53 | 8331 | 9216 | 9114 | −3392 | −1299 | 990 | C |
| ATOM | 416 | CD | PRO | C | 53 | 83.503 | 17.889 | 4.243 | 1.00 | 66.55 | | C |
| ANISOU | 416 | CD | PRO | C | 53 | 7708 | 8830 | 8747 | −3300 | −1299 | 1088 | C |
| ATOM | 417 | N | VAL | C | 54 | 82.758 | 18.884 | 0.673 | 1.00 | 63.84 | | N |
| ANISOU | 417 | N | VAL | C | 54 | 7336 | 8236 | 8683 | −3343 | −809 | 1308 | N |
| ATOM | 418 | CA | VAL | C | 54 | 82.870 | 19.331 | −0.724 | 1.00 | 62.79 | | C |
| ANISOU | 418 | CA | VAL | C | 54 | 7134 | 8056 | 8667 | −3371 | −638 | 1411 | C |
| ATOM | 419 | C | VAL | C | 54 | 82.559 | 18.143 | −1.648 | 1.00 | 64.62 | | C |
| ANISOU | 419 | C | VAL | C | 54 | 7425 | 8329 | 8798 | −3187 | −534 | 1509 | C |
| ATOM | 420 | O | VAL | C | 54 | 81.674 | 18.259 | −2.491 | 1.00 | 62.82 | | O |
| ANISOU | 420 | O | VAL | C | 54 | 7316 | 8012 | 8540 | −3161 | −404 | 1558 | O |
| ATOM | 421 | CB | VAL | C | 54 | 84.259 | 19.959 | −1.013 | 1.00 | 68.33 | | C |
| ANISOU | 421 | CB | VAL | C | 54 | 7570 | 8847 | 9545 | −3492 | −639 | 1433 | C |
| ATOM | 422 | CG1 | VAL | C | 54 | 84.577 | 19.982 | −2.505 | 1.00 | 67.95 | | C |
| ANISOU | 422 | CG1 | VAL | C | 54 | 7422 | 8835 | 9562 | −3486 | −457 | 1570 | C |
| ATOM | 423 | CG2 | VAL | C | 54 | 84.358 | 21.353 | −0.418 | 1.00 | 69.29 | | C |
| ANISOU | 423 | CG2 | VAL | C | 54 | 7671 | 8860 | 9797 | −3707 | −712 | 1345 | C |
| ATOM | 424 | N | ALA | C | 55 | 83.240 | 16.987 | −1.432 | 1.00 | 61.02 | | N |
| ANISOU | 424 | N | ALA | C | 55 | 6903 | 8001 | 8283 | −3044 | −613 | 1520 | N |
| ATOM | 425 | CA | ALA | C | 55 | 83.074 | 15.740 | −2.195 | 1.00 | 59.30 | | C |
| ANISOU | 425 | CA | ALA | C | 55 | 6744 | 7813 | 7974 | −2848 | −557 | 1587 | C |
| ATOM | 426 | C | ALA | C | 55 | 81.654 | 15.226 | −2.137 | 1.00 | 58.33 | | C |
| ANISOU | 426 | C | ALA | C | 55 | 6888 | 7568 | 7705 | −2806 | −525 | 1591 | C |
| ATOM | 427 | O | ALA | C | 55 | 81.159 | 14.665 | −3.114 | 1.00 | 57.00 | | O |
| ANISOU | 427 | O | ALA | C | 55 | 6789 | 7375 | 7496 | −2704 | −414 | 1645 | O |
| ATOM | 428 | CB | ALA | C | 55 | 84.024 | 14.677 | −1.680 | 1.00 | 61.42 | | C |
| ANISOU | 428 | CB | ALA | C | 55 | 6932 | 8203 | 8203 | −2697 | −707 | 1569 | C |
| ATOM | 429 | N | VAL | C | 56 | 81.006 | 15.419 | −0.985 | 1.00 | 52.60 | | N |
| ANISOU | 429 | N | VAL | C | 56 | 6299 | 6793 | 6894 | −2892 | −621 | 1522 | N |
| ATOM | 430 | CA | VAL | C | 56 | 79.617 | 15.048 | −0.759 | 1.00 | 49.74 | | C |
| ANISOU | 430 | CA | VAL | C | 56 | 6166 | 6356 | 6377 | −2896 | −593 | 1506 | C |
| ATOM | 431 | C | VAL | C | 56 | 78.727 | 15.985 | −1.613 | 1.00 | 54.37 | | C |
| ANISOU | 431 | C | VAL | C | 56 | 6786 | 6863 | 7011 | −2955 | −436 | 1496 | C |
| ATOM | 432 | O | VAL | C | 56 | 77.841 | 15.501 | −2.319 | 1.00 | 53.57 | | O |
| ANISOU | 432 | O | VAL | C | 56 | 6796 | 6726 | 6831 | −2888 | −335 | 1525 | O |
| ATOM | 433 | CB | VAL | C | 56 | 79.273 | 15.053 | 0.757 | 1.00 | 51.23 | | C |
| ANISOU | 433 | CB | VAL | C | 56 | 6456 | 6571 | 6438 | −2979 | −740 | 1427 | C |
| ATOM | 434 | CG1 | VAL | C | 56 | 77.768 | 15.088 | 0.990 | 1.00 | 49.06 | | C |
| ANISOU | 434 | CG1 | VAL | C | 56 | 6368 | 6261 | 6013 | −3038 | −684 | 1378 | C |
| ATOM | 435 | CG2 | VAL | C | 56 | 79.903 | 13.856 | 1.457 | 1.00 | 51.36 | | C |
| ANISOU | 435 | CG2 | VAL | C | 56 | 6512 | 6642 | 6359 | −2886 | −892 | 1471 | C |
| ATOM | 436 | N | ALA | C | 57 | 79.011 | 17.305 | −1.599 | 1.00 | 51.65 | | N |
| ANISOU | 436 | N | ALA | C | 57 | 6347 | 6480 | 6799 | −3072 | −427 | 1456 | N |
| ATOM | 437 | CA | ALA | C | 57 | 78.253 | 18.284 | −2.373 | 1.00 | 51.28 | | C |
| ANISOU | 437 | CA | ALA | C | 57 | 6348 | 6331 | 6806 | −3116 | −311 | 1451 | C |
| ATOM | 438 | C | ALA | C | 57 | 78.372 | 17.996 | −3.855 | 1.00 | 57.49 | | C |
| ANISOU | 438 | C | ALA | C | 57 | 7093 | 7121 | 7628 | −3027 | −158 | 1571 | C |
| ATOM | 439 | O | ALA | C | 57 | 77.397 | 18.216 | −4.583 | 1.00 | 57.13 | | O |
| ANISOU | 439 | O | ALA | C | 57 | 7152 | 7014 | 7540 | −2991 | −56 | 1579 | O |
| ATOM | 440 | CB | ALA | C | 57 | 78.731 | 19.682 | −2.070 | 1.00 | 53.39 | | C |
| ANISOU | 440 | CB | ALA | C | 57 | 6528 | 6529 | 7231 | −3259 | −359 | 1405 | C |
| ATOM | 441 | N | GLU | C | 58 | 79.558 | 17.457 | −4.290 | 1.00 | 55.03 | | N |
| ANISOU | 441 | N | GLU | C | 58 | 6620 | 6909 | 7379 | −2975 | −149 | 1649 | N |
| ATOM | 442 | CA | GLU | C | 58 | 79.861 | 17.032 | −5.663 | 1.00 | 53.75 | | C |
| ANISOU | 442 | CA | GLU | C | 58 | 6381 | 6808 | 7232 | −2872 | −12 | 1750 | C |
| ATOM | 443 | C | GLU | C | 58 | 78.905 | 15.920 | −6.038 | 1.00 | 55.42 | | C |
| ANISOU | 443 | C | GLU | C | 58 | 6751 | 7009 | 7298 | −2725 | 30 | 1744 | C |
| ATOM | 444 | O | GLU | C | 58 | 78.348 | 15.966 | −7.130 | 1.00 | 55.14 | | O |
| ANISOU | 444 | O | GLU | C | 58 | 6753 | 6958 | 7239 | −2671 | 161 | 1787 | O |
| ATOM | 445 | CB | GLU | C | 58 | 81.300 | 16.525 | −5.791 | 1.00 | 56.41 | | C |
| ANISOU | 445 | CB | GLU | C | 58 | 6501 | 7300 | 7632 | −2820 | −45 | 1784 | C |
| ATOM | 446 | CG | GLU | C | 58 | 82.319 | 17.623 | −5.994 | 1.00 | 71.82 | | C |
| ANISOU | 446 | CG | GLU | C | 58 | 8245 | 9306 | 9737 | −2977 | −13 | 1825 | C |
| ATOM | 447 | CD | GLU | C | 58 | 83.708 | 17.155 | −6.386 | 1.00 | 109.30 | | C |
| ANISOU | 447 | CD | GLU | C | 58 | 12732 | 14265 | 14532 | −2920 | −8 | 1852 | C |
| ATOM | 448 | OE1 | GLU | C | 58 | 84.063 | 15.992 | −6.082 | 1.00 | 108.93 | | O |
| ANISOU | 448 | OE1 | GLU | C | 58 | 12666 | 14308 | 14414 | −2741 | −92 | 1808 | O |
| ATOM | 449 | OE2 | GLU | C | 58 | 84.453 | 17.967 | −6.981 | 1.00 | 111.32 | | O |
| ANISOU | 449 | OE2 | GLU | C | 58 | 12801 | 14601 | 14893 | −3058 | 71 | 1914 | O |
| ATOM | 450 | N | ILE | C | 59 | 78.682 | 14.940 | −5.127 | 1.00 | 50.57 | | N |
| ANISOU | 450 | N | ILE | C | 59 | 6241 | 6396 | 6579 | −2674 | −88 | 1695 | N |
| ATOM | 451 | CA | ILE | C | 59 | 77.751 | 13.845 | −5.371 | 1.00 | 49.28 | | C |
| ANISOU | 451 | CA | ILE | C | 59 | 6246 | 6201 | 6276 | −2573 | −66 | 1688 | C |
| ATOM | 452 | C | ILE | C | 59 | 76.388 | 14.444 | −5.695 | 1.00 | 53.98 | | C |
| ANISOU | 452 | C | ILE | C | 59 | 6962 | 6735 | 6812 | −2628 | 32 | 1650 | C |
| ATOM | 453 | O | ILE | C | 59 | 75.883 | 14.200 | −6.788 | 1.00 | 54.04 | | O |
| ANISOU | 453 | O | ILE | C | 59 | 6997 | 6743 | 6792 | −2544 | 148 | 1676 | O |
| ATOM | 454 | CB | ILE | C | 59 | 77.696 | 12.808 | −4.217 | 1.00 | 52.44 | | C |
| ANISOU | 454 | CB | ILE | C | 59 | 6771 | 6588 | 6564 | −2558 | −222 | 1662 | C |
| ATOM | 455 | CG1 | ILE | C | 59 | 79.036 | 12.047 | −4.079 | 1.00 | 53.87 | | C |
| ANISOU | 455 | CG1 | ILE | C | 59 | 6841 | 6830 | 6796 | −2440 | −333 | 1694 | C |
| ATOM | 456 | CG2 | ILE | C | 59 | 76.510 | 11.834 | −4.407 | 1.00 | 52.81 | | C |
| ANISOU | 456 | CG2 | ILE | C | 59 | 7022 | 6579 | 6463 | −2520 | −193 | 1655 | C |
| ATOM | 457 | CD1 | ILE | C | 59 | 79.146 | 11.098 | −2.828 | 1.00 | 58.83 | | C |
| ANISOU | 457 | CD1 | ILE | C | 59 | 7597 | 7434 | 7320 | −2430 | −527 | 1687 | C |
| ATOM | 458 | N | LEU | C | 60 | 75.861 | 15.299 | −4.795 | 1.00 | 51.12 | | N |
| ANISOU | 458 | N | LEU | C | 60 | 6651 | 6338 | 6436 | −2753 | −19 | 1575 | N |
| ATOM | 459 | CA | LEU | C | 60 | 74.578 | 16.001 | −4.912 | 1.00 | 50.74 | | C |
| ANISOU | 459 | CA | LEU | C | 60 | 6702 | 6248 | 6330 | −2792 | 42 | 1501 | C |
| ATOM | 460 | C | LEU | C | 60 | 74.471 | 16.831 | −6.186 | 1.00 | 58.63 | | C |
| ANISOU | 460 | C | LEU | C | 60 | 7656 | 7203 | 7417 | −2753 | 169 | 1553 | C |
| ATOM | 461 | O | LEU | C | 60 | 73.423 | 16.805 | −6.833 | 1.00 | 58.75 | | O |
| ANISOU | 461 | O | LEU | C | 60 | 7757 | 7213 | 7353 | −2694 | 252 | 1525 | O |
| ATOM | 462 | CB | LEU | C | 60 | 74.337 | 16.890 | −3.689 | 1.00 | 51.06 | | C |
| ANISOU | 462 | CB | LEU | C | 60 | 6761 | 6274 | 6364 | −2912 | −59 | 1391 | C |
| ATOM | 463 | CG | LEU | C | 60 | 74.486 | 16.241 | −2.308 | 1.00 | 55.11 | | C |
| ANISOU | 463 | CG | LEU | C | 60 | 7316 | 6853 | 6771 | −2971 | −194 | 1343 | C |
| ATOM | 464 | CD1 | LEU | C | 60 | 74.793 | 17.273 | −1.277 | 1.00 | 55.62 | | C |
| ANISOU | 464 | CD1 | LEU | C | 60 | 7323 | 6916 | 6894 | −3072 | −302 | 1253 | C |
| ATOM | 465 | CD2 | LEU | C | 60 | 73.241 | 15.498 | −1.914 | 1.00 | 56.82 | | C |
| ANISOU | 465 | CD2 | LEU | C | 60 | 7679 | 7127 | 6784 | −2990 | −184 | 1278 | C |
| ATOM | 466 | N | ALA | C | 61 | 75.558 | 17.534 | −6.570 | 1.00 | 57.71 | | N |
| ANISOU | 466 | N | ALA | C | 61 | 7404 | 7071 | 7453 | −2791 | 185 | 1636 | N |
| ATOM | 467 | CA | ALA | C | 61 | 75.637 | 18.318 | −7.808 | 1.00 | 58.40 | | C |
| ANISOU | 467 | CA | ALA | C | 61 | 7449 | 7122 | 7619 | −2778 | 304 | 1727 | C |
| ATOM | 468 | C | ALA | C | 61 | 75.601 | 17.392 | −9.050 | 1.00 | 64.54 | | C |
| ANISOU | 468 | C | ALA | C | 61 | 8205 | 7988 | 8330 | −2634 | 426 | 1802 | C |
| ATOM | 469 | O | ALA | C | 61 | 75.058 | 17.765 | −10.091 | 1.00 | 63.50 | | O |
| ANISOU | 469 | O | ALA | C | 61 | 8107 | 7842 | 8177 | −2582 | 532 | 1848 | O |
| ATOM | 470 | CB | ALA | C | 61 | 76.906 | 19.154 | −7.810 | 1.00 | 60.64 | | C |
| ANISOU | 470 | CB | ALA | C | 61 | 7582 | 7393 | 8066 | −2897 | 286 | 1805 | C |
| ATOM | 471 | N | GLY | C | 62 | 76.182 | 16.199 | −8.919 | 1.00 | 64.16 | | N |
| ANISOU | 471 | N | GLY | C | 62 | 8105 | 8027 | 8246 | −2557 | 393 | 1805 | N |
| ATOM | 472 | CA | GLY | C | 62 | 76.195 | 15.186 | −9.969 | 1.00 | 65.40 | | C |
| ANISOU | 472 | CA | GLY | C | 62 | 8242 | 8267 | 8339 | −2401 | 478 | 1840 | C |
| ATOM | 473 | C | GLY | C | 62 | 74.838 | 14.511 | −10.082 | 1.00 | 71.98 | | C |
| ANISOU | 473 | C | GLY | C | 62 | 9248 | 9067 | 9033 | −2333 | 496 | 1764 | C |
| ATOM | 474 | O | GLY | C | 62 | 74.499 | 13.938 | −11.124 | 1.00 | 72.91 | | O |
| ANISOU | 474 | O | GLY | C | 62 | 9378 | 9233 | 9090 | −2210 | 582 | 1773 | O |
| ATOM | 475 | N | LEU | C | 63 | 74.054 | 14.591 | −8.991 | 1.00 | 68.24 | | N |
| ANISOU | 475 | N | LEU | C | 63 | 8896 | 8533 | 8500 | −2422 | 413 | 1678 | N |
| ATOM | 476 | CA | LEU | C | 63 | 72.694 | 14.082 | −8.846 | 1.00 | 66.91 | | C |
| ANISOU | 476 | CA | LEU | C | 63 | 8877 | 8357 | 8188 | −2415 | 421 | 1588 | C |
| ATOM | 477 | C | LEU | C | 63 | 71.685 | 15.190 | −9.208 | 1.00 | 72.53 | | C |
| ANISOU | 477 | C | LEU | C | 63 | 9627 | 9050 | 8880 | −2432 | 488 | 1536 | C |
| ATOM | 478 | O | LEU | C | 63 | 70.479 | 14.936 | −9.203 | 1.00 | 72.27 | | O |
| ANISOU | 478 | O | LEU | C | 63 | 9688 | 9046 | 8725 | −2420 | 510 | 1442 | O |
| ATOM | 479 | CB | LEU | C | 63 | 72.485 | 13.646 | −7.383 | 1.00 | 66.55 | | C |
| ANISOU | 479 | CB | LEU | C | 63 | 8917 | 8300 | 8071 | −2523 | 293 | 1525 | C |
| ATOM | 480 | CG | LEU | C | 63 | 72.518 | 12.165 | −7.059 | 1.00 | 70.18 | | C |
| ANISOU | 480 | CG | LEU | C | 63 | 9464 | 8755 | 8445 | −2498 | 221 | 1537 | C |
| ATOM | 481 | CD1 | LEU | C | 63 | 73.905 | 11.566 | −7.284 | 1.00 | 71.09 | | C |
| ANISOU | 481 | CD1 | LEU | C | 63 | 9485 | 8868 | 8658 | −2391 | 173 | 1618 | C |
| ATOM | 482 | CD2 | LEU | C | 63 | 72.126 | 11.950 | −5.634 | 1.00 | 71.37 | | C |
| ANISOU | 482 | CD2 | LEU | C | 63 | 9707 | 8908 | 8501 | −2633 | 105 | 1493 | C |
| ATOM | 483 | N | GLN | C | 64 | 72.181 | 16.412 | −9.529 | 1.00 | 70.69 | | N |
| ANISOU | 483 | N | GLN | C | 64 | 9324 | 8768 | 8766 | −2460 | 510 | 1594 | N |
| ATOM | 484 | CA | GLN | C | 64 | 71.397 | 17.600 | −9.910 | 1.00 | 71.31 | | C |
| ANISOU | 484 | CA | GLN | C | 64 | 9453 | 8787 | 8853 | −2456 | 544 | 1562 | C |
| ATOM | 485 | C | GLN | C | 64 | 70.486 | 18.089 | −8.785 | 1.00 | 77.41 | | C |
| ANISOU | 485 | C | GLN | C | 64 | 10307 | 9536 | 9568 | −2521 | 454 | 1405 | C |
| ATOM | 486 | O | GLN | C | 64 | 69.410 | 18.621 | −9.064 | 1.00 | 76.85 | | O |
| ANISOU | 486 | O | GLN | C | 64 | 10304 | 9461 | 9435 | −2465 | 474 | 1316 | O |
| ATOM | 487 | CB | GLN | C | 64 | 70.587 | 17.370 | −11.211 | 1.00 | 72.04 | | C |
| ANISOU | 487 | CB | GLN | C | 64 | 9586 | 8929 | 8859 | −2320 | 659 | 1572 | C |
| ATOM | 488 | CG | GLN | C | 64 | 71.403 | 17.347 | −12.501 | 1.00 | 87.33 | | C |
| ANISOU | 488 | CG | GLN | C | 64 | 11434 | 10900 | 10848 | −2250 | 762 | 1726 | C |
| ATOM | 489 | CD | GLN | C | 64 | 71.168 | 18.565 | −13.366 | 1.00 | 103.54 | | C |
| ANISOU | 489 | CD | GLN | C | 64 | 13515 | 12891 | 12933 | −2225 | 818 | 1802 | C |
| ATOM | 490 | NE2 | GLN | C | 64 | 72.217 | 19.004 | −14.054 | 1.00 | 95.82 | | N |
| ANISOU | 490 | NE2 | GLN | C | 64 | 12446 | 11919 | 12042 | −2261 | 878 | 1965 | N |
| ATOM | 491 | OE1 | GLN | C | 64 | 70.055 | 19.110 | −13.448 | 1.00 | 96.89 | | O |
| ANISOU | 491 | OE1 | GLN | C | 64 | 12778 | 12008 | 12029 | −2172 | 805 | 1717 | O |
| ATOM | 492 | N | MET | C | 65 | 70.924 | 17.930 | −7.523 | 1.00 | 76.73 | | N |
| ANISOU | 492 | N | MET | C | 65 | 10205 | 9456 | 9492 | −2626 | 348 | 1359 | N |
| ATOM | 493 | CA | MET | C | 65 | 70.154 | 18.297 | −6.327 | 1.00 | 77.91 | | C |
| ANISOU | 493 | CA | MET | C | 65 | 10410 | 9630 | 9562 | −2694 | 257 | 1197 | C |
| ATOM | 494 | C | MET | C | 65 | 69.929 | 19.812 | −6.155 | 1.00 | 85.78 | | C |
| ANISOU | 494 | C | MET | C | 65 | 11420 | 10525 | 10646 | −2704 | 205 | 1118 | C |
| ATOM | 495 | O | MET | C | 65 | 70.687 | 20.624 | −6.695 | 1.00 | 85.98 | | O |
| ANISOU | 495 | O | MET | C | 65 | 11413 | 10428 | 10829 | −2716 | 210 | 1219 | O |
| ATOM | 496 | CB | MET | C | 65 | 70.766 | 17.678 | −5.057 | 1.00 | 80.59 | | C |
| ANISOU | 496 | CB | MET | C | 65 | 10730 | 10017 | 9873 | −2797 | 153 | 1186 | C |
| ATOM | 497 | CG | MET | C | 65 | 70.682 | 16.157 | −5.031 | 1.00 | 83.76 | | C |
| ANISOU | 497 | CG | MET | C | 65 | 11178 | 10496 | 10150 | −2789 | 167 | 1228 | C |
| ATOM | 498 | SD | MET | C | 65 | 68.977 | 15.552 | −5.046 | 1.00 | 87.20 | | S |
| ANISOU | 498 | SD | MET | C | 65 | 11719 | 11043 | 10370 | −2788 | 228 | 1104 | S |
| ATOM | 499 | CE | MET | C | 65 | 69.120 | 14.192 | −6.185 | 1.00 | 83.18 | | C |
| ANISOU | 499 | CE | MET | C | 65 | 11244 | 10521 | 9841 | −2702 | 304 | 1219 | C |
| ATOM | 500 | N | ASP | C | 66 | 68.852 | 20.170 | −5.412 | 1.00 | 84.92 | | N |
| ANISOU | 500 | N | ASP | C | 66 | 11364 | 10476 | 10426 | −2702 | 150 | 929 | N |
| ATOM | 501 | CA | ASP | C | 66 | 68.395 | 21.538 | −5.123 | 1.00 | 86.93 | | C |
| ANISOU | 501 | CA | ASP | C | 66 | 11652 | 10643 | 10733 | −2676 | 70 | 791 | C |
| ATOM | 502 | C | ASP | C | 66 | 69.478 | 22.400 | −4.474 | 1.00 | 92.48 | | C |
| ANISOU | 502 | C | ASP | C | 66 | 12319 | 11208 | 11613 | −2777 | −37 | 816 | C |
| ATOM | 503 | O | ASP | C | 66 | 70.306 | 21.880 | −3.721 | 1.00 | 93.09 | | O |
| ANISOU | 503 | O | ASP | C | 66 | 12331 | 11332 | 11706 | −2878 | −79 | 859 | O |
| ATOM | 504 | CB | ASP | C | 66 | 67.148 | 21.492 | −4.215 | 1.00 | 89.57 | | C |
| ANISOU | 504 | CB | ASP | C | 66 | 12007 | 11143 | 10881 | −2667 | 20 | 554 | C |
| ATOM | 505 | CG | ASP | C | 66 | 66.278 | 22.742 | −4.228 | 1.00 | 109.37 | | C |
| ANISOU | 505 | CG | ASP | C | 66 | 14559 | 13604 | 13392 | −2557 | −46 | 366 | C |
| ATOM | 506 | OD1 | ASP | C | 66 | 66.780 | 23.829 | −3.831 | 1.00 | 112.01 | | O |
| ANISOU | 506 | OD1 | ASP | C | 66 | 14911 | 13773 | 13876 | −2570 | −154 | 328 | O |
| ATOM | 507 | OD2 | ASP | C | 66 | 65.076 | 22.625 | −4.571 | 1.00 | 118.19 | | O |
| ANISOU | 507 | OD2 | ASP | C | 66 | 15691 | 14855 | 14359 | −2456 | −3 | 236 | O |
| ATOM | 508 | N | ALA | C | 67 | 69.440 | 23.724 | −4.732 | 1.00 | 88.91 | | N |
| ANISOU | 508 | N | ALA | C | 67 | 11916 | 10577 | 11290 | −2748 | −96 | 780 | N |
| ATOM | 509 | CA | ALA | C | 67 | 70.375 | 24.703 | −4.163 | 1.00 | 89.64 | | C |
| ANISOU | 509 | CA | ALA | C | 67 | 11987 | 10508 | 11566 | −2860 | −212 | 782 | C |
| ATOM | 510 | C | ALA | C | 67 | 70.576 | 24.518 | −2.659 | 1.00 | 91.32 | | C |
| ANISOU | 510 | C | ALA | C | 67 | 12144 | 10830 | 11725 | −2947 | −326 | 629 | C |
| ATOM | 511 | O | ALA | C | 67 | 71.707 | 24.565 | −2.179 | 1.00 | 91.31 | | O |
| ANISOU | 511 | O | ALA | C | 67 | 12065 | 10795 | 11834 | −3068 | −382 | 692 | O |
| ATOM | 512 | CB | ALA | C | 67 | 69.868 | 26.104 | −4.427 | 1.00 | 91.99 | | C |
| ANISOU | 512 | CB | ALA | C | 67 | 12393 | 10598 | 11961 | −2794 | −296 | 695 | C |
| ATOM | 513 | N | ASP | C | 68 | 69.471 | 24.288 | −1.933 | 1.00 | 86.25 | | N |
| ANISOU | 513 | N | ASP | C | 68 | 11527 | 10350 | 10894 | −2888 | −357 | 428 | N |
| ATOM | 514 | CA | ASP | C | 68 | 69.448 | 24.091 | −0.487 | 1.00 | 85.82 | | C |
| ANISOU | 514 | CA | ASP | C | 68 | 11428 | 10451 | 10729 | −2960 | −457 | 266 | C |
| ATOM | 515 | C | ASP | C | 68 | 70.070 | 22.753 | −0.068 | 1.00 | 83.67 | | C |
| ANISOU | 515 | C | ASP | C | 68 | 11099 | 10329 | 10362 | −3051 | −418 | 396 | C |
| ATOM | 516 | O | ASP | C | 68 | 70.690 | 22.694 | 0.991 | 1.00 | 82.97 | | O |
| ANISOU | 516 | O | ASP | C | 68 | 10961 | 10300 | 10263 | −3140 | −518 | 352 | O |
| ATOM | 517 | CB | ASP | C | 68 | 68.012 | 24.235 | 0.059 | 1.00 | 88.74 | | C |
| ANISOU | 517 | CB | ASP | C | 68 | 11828 | 10991 | 10899 | −2873 | −483 | 13 | C |
| ATOM | 518 | CG | ASP | C | 68 | 67.336 | 25.565 | −0.256 | 1.00 | 105.78 | | C |
| ANISOU | 518 | CG | ASP | C | 68 | 14050 | 13001 | 13142 | −2744 | −564 | −159 | C |
| ATOM | 519 | OD1 | ASP | C | 68 | 67.497 | 26.522 | 0.543 | 1.00 | 107.50 | | O |
| ANISOU | 519 | OD1 | ASP | C | 68 | 14264 | 13158 | 13422 | −2755 | −712 | −336 | O |
| ATOM | 520 | OD2 | ASP | C | 68 | 66.615 | 25.637 | −1.281 | 1.00 | 114.49 | | O |
| ANISOU | 520 | OD2 | ASP | C | 68 | 15211 | 14048 | 14241 | −2619 | −492 | −129 | O |
| ATOM | 521 | N | THR | C | 69 | 69.915 | 21.695 | −0.899 | 1.00 | 76.14 | | N |
| ANISOU | 521 | N | THR | C | 69 | 10162 | 9428 | 9340 | −3016 | −290 | 546 | N |
| ATOM | 522 | CA | THR | C | 69 | 70.464 | 20.353 | −0.637 | 1.00 | 74.26 | | C |
| ANISOU | 522 | CA | THR | C | 69 | 9903 | 9292 | 9019 | −3073 | −266 | 677 | C |
| ATOM | 523 | C | THR | C | 69 | 71.976 | 20.327 | −0.824 | 1.00 | 75.56 | | C |
| ANISOU | 523 | C | THR | C | 69 | 9987 | 9356 | 9367 | −3115 | −296 | 835 | C |
| ATOM | 524 | O | THR | C | 69 | 72.663 | 19.640 | −0.069 | 1.00 | 75.08 | | O |
| ANISOU | 524 | O | THR | C | 69 | 9890 | 9368 | 9267 | −3171 | −364 | 874 | O |
| ATOM | 525 | CB | THR | C | 69 | 69.809 | 19.284 | −1.527 | 1.00 | 83.34 | | C |
| ANISOU | 525 | CB | THR | C | 69 | 11105 | 10502 | 10059 | −3011 | −136 | 764 | C |
| ATOM | 526 | CG2 | THR | C | 69 | 69.800 | 17.901 | −0.877 | 1.00 | 80.71 | | C |
| ANISOU | 526 | CG2 | THR | C | 69 | 10805 | 10291 | 9570 | −3076 | −148 | 826 | C |
| ATOM | 527 | OG1 | THR | C | 69 | 68.482 | 19.677 | −1.862 | 1.00 | 86.03 | | O |
| ANISOU | 527 | OG1 | THR | C | 69 | 11492 | 10908 | 10289 | −2947 | −87 | 621 | O |
| ATOM | 528 | N | VAL | C | 70 | 72.488 | 21.050 | −1.846 | 1.00 | 70.47 | | N |
| ANISOU | 528 | N | VAL | C | 70 | 9309 | 8560 | 8908 | −3091 | −246 | 928 | N |
| ATOM | 529 | CA | VAL | C | 70 | 73.923 | 21.149 | −2.142 | 1.00 | 70.12 | | C |
| ANISOU | 529 | CA | VAL | C | 70 | 9154 | 8449 | 9038 | −3148 | −256 | 1069 | C |
| ATOM | 530 | C | VAL | C | 70 | 74.612 | 21.913 | −0.984 | 1.00 | 74.41 | | C |
| ANISOU | 530 | C | VAL | C | 70 | 9636 | 8969 | 9666 | −3260 | −409 | 965 | C |
| ATOM | 531 | O | VAL | C | 70 | 75.611 | 21.429 | −0.433 | 1.00 | 74.65 | | O |
| ANISOU | 531 | O | VAL | C | 70 | 9574 | 9073 | 9718 | −3311 | −472 | 1007 | O |
| ATOM | 532 | CB | VAL | C | 70 | 74.201 | 21.771 | −3.537 | 1.00 | 73.82 | | C |
| ANISOU | 532 | CB | VAL | C | 70 | 9606 | 8790 | 9652 | −3126 | −153 | 1202 | C |
| ATOM | 533 | CG1 | VAL | C | 70 | 75.692 | 21.784 | −3.842 | 1.00 | 74.72 | | C |
| ANISOU | 533 | CG1 | VAL | C | 70 | 9572 | 8900 | 9917 | −3205 | −147 | 1344 | C |
| ATOM | 534 | CG2 | VAL | C | 70 | 73.456 | 21.019 | −4.640 | 1.00 | 72.20 | | C |
| ANISOU | 534 | CG2 | VAL | C | 70 | 9460 | 8632 | 9342 | −3000 | −13 | 1275 | C |
| ATOM | 535 | N | ALA | C | 71 | 74.013 | 23.062 | −0.572 | 1.00 | 69.75 | | N |
| ANISOU | 535 | N | ALA | C | 71 | 9103 | 8289 | 9111 | −3278 | −482 | 806 | N |
| ATOM | 536 | CA | ALA | C | 71 | 74.460 | 23.904 | 0.538 | 1.00 | 69.83 | | C |
| ANISOU | 536 | CA | ALA | C | 71 | 9072 | 8266 | 9194 | −3371 | −640 | 659 | C |
| ATOM | 537 | C | ALA | C | 71 | 74.447 | 23.098 | 1.824 | 1.00 | 72.72 | | C |
| ANISOU | 537 | C | ALA | C | 71 | 9413 | 8835 | 9383 | −3390 | −724 | 569 | C |
| ATOM | 538 | O | ALA | C | 71 | 75.316 | 23.294 | 2.670 | 1.00 | 73.03 | | O |
| ANISOU | 538 | O | ALA | C | 71 | 9368 | 8902 | 9477 | −3472 | −843 | 521 | O |
| ATOM | 539 | CB | ALA | C | 71 | 73.547 | 25.104 | 0.674 | 1.00 | 71.11 | | C |
| ANISOU | 539 | CB | ALA | C | 71 | 9329 | 8305 | 9383 | −3333 | −704 | 476 | C |
| ATOM | 540 | N | ALA | C | 72 | 73.479 | 22.161 | 1.946 | 1.00 | 68.09 | | N |
| ANISOU | 540 | N | ALA | C | 72 | 8900 | 8396 | 8575 | −3327 | −662 | 555 | N |
| ATOM | 541 | CA | ALA | C | 72 | 73.325 | 21.259 | 3.091 | 1.00 | 67.70 | | C |
| ANISOU | 541 | CA | ALA | C | 72 | 8861 | 8548 | 8316 | −3359 | −725 | 508 | C |
| ATOM | 542 | C | ALA | C | 72 | 74.403 | 20.185 | 3.097 | 1.00 | 70.06 | | C |
| ANISOU | 542 | C | ALA | C | 72 | 9111 | 8884 | 8624 | −3373 | −737 | 687 | C |
| ATOM | 543 | O | ALA | C | 72 | 74.805 | 19.727 | 4.171 | 1.00 | 70.41 | | O |
| ANISOU | 543 | O | ALA | C | 72 | 9140 | 9048 | 8565 | −3417 | −846 | 664 | O |
| ATOM | 544 | CB | ALA | C | 72 | 71.953 | 20.612 | 3.072 | 1.00 | 67.44 | | C |
| ANISOU | 544 | CB | ALA | C | 72 | 8924 | 8647 | 8052 | −3319 | −644 | 457 | C |
| ATOM | 545 | N | GLY | C | 73 | 74.857 | 19.800 | 1.902 | 1.00 | 64.45 | | N |
| ANISOU | 545 | N | GLY | C | 73 | 8377 | 8085 | 8025 | −3321 | −634 | 854 | N |
| ATOM | 546 | CA | GLY | C | 73 | 75.918 | 18.817 | 1.732 | 1.00 | 63.88 | | C |
| ANISOU | 546 | CA | GLY | C | 73 | 8244 | 8045 | 7983 | −3294 | −647 | 1006 | C |
| ATOM | 547 | C | GLY | C | 73 | 77.231 | 19.312 | 2.295 | 1.00 | 68.05 | | C |
| ANISOU | 547 | C | GLY | C | 73 | 8627 | 8577 | 8652 | −3357 | −765 | 991 | C |
| ATOM | 548 | O | GLY | C | 73 | 77.970 | 18.549 | 2.922 | 1.00 | 68.33 | | O |
| ANISOU | 548 | O | GLY | C | 73 | 8622 | 8706 | 8634 | −3343 | −861 | 1028 | O |
| ATOM | 549 | N | LEU | C | 74 | 77.505 | 20.608 | 2.111 | 1.00 | 64.38 | | N |
| ANISOU | 549 | N | LEU | C | 74 | 8091 | 8006 | 8363 | −3429 | −774 | 928 | N |
| ATOM | 550 | CA | LEU | C | 74 | 78.719 | 21.238 | 2.599 | 1.00 | 65.73 | | C |
| ANISOU | 550 | CA | LEU | C | 74 | 8111 | 8174 | 8690 | −3523 | −884 | 894 | C |
| ATOM | 551 | C | LEU | C | 74 | 78.642 | 21.502 | 4.095 | 1.00 | 74.71 | | C |
| ANISOU | 551 | C | LEU | C | 74 | 9255 | 9398 | 9735 | −3575 | −1050 | 720 | C |
| ATOM | 552 | O | LEU | C | 74 | 79.684 | 21.654 | 4.726 | 1.00 | 77.55 | | O |
| ANISOU | 552 | O | LEU | C | 74 | 9485 | 9813 | 10167 | −3634 | −1168 | 686 | O |
| ATOM | 553 | CB | LEU | C | 74 | 79.000 | 22.545 | 1.846 | 1.00 | 66.04 | | C |
| ANISOU | 553 | CB | LEU | C | 74 | 8101 | 8042 | 8951 | −3613 | −841 | 899 | C |
| ATOM | 554 | CG | LEU | C | 74 | 79.079 | 22.477 | 0.316 | 1.00 | 68.66 | | C |
| ANISOU | 554 | CG | LEU | C | 74 | 8419 | 8299 | 9369 | −3580 | −672 | 1076 | C |
| ATOM | 555 | CD1 | LEU | C | 74 | 78.551 | 23.742 | −0.301 | 1.00 | 68.17 | | C |
| ANISOU | 555 | CD1 | LEU | C | 74 | 8436 | 8037 | 9427 | −3636 | −630 | 1061 | C |
| ATOM | 556 | CD2 | LEU | C | 74 | 80.496 | 22.199 | −0.157 | 1.00 | 71.53 | | C |
| ANISOU | 556 | CD2 | LEU | C | 74 | 8586 | 8741 | 9850 | −3622 | −651 | 1199 | C |
| ATOM | 557 | N | LEU | C | 75 | 77.429 | 21.530 | 4.686 | 1.00 | 71.75 | | N |
| ANISOU | 557 | N | LEU | C | 75 | 9010 | 9068 | 9184 | −3553 | −1063 | 598 | N |
| ATOM | 558 | CA | LEU | C | 75 | 77.289 | 21.835 | 6.122 | 1.00 | 72.85 | | C |
| ANISOU | 558 | CA | LEU | C | 75 | 9147 | 9325 | 9206 | −3600 | −1216 | 413 | C |
| ATOM | 559 | C | LEU | C | 75 | 76.736 | 20.699 | 7.000 | 1.00 | 75.57 | | C |
| ANISOU | 559 | C | LEU | C | 75 | 9580 | 9870 | 9262 | −3570 | −1248 | 421 | C |
| ATOM | 560 | O | LEU | C | 75 | 76.541 | 20.915 | 8.196 | 1.00 | 76.16 | | O |
| ANISOU | 560 | O | LEU | C | 75 | 9652 | 10081 | 9203 | −3610 | −1368 | 274 | O |
| ATOM | 561 | CB | LEU | C | 75 | 76.438 | 23.104 | 6.308 | 1.00 | 73.63 | | C |
| ANISOU | 561 | CB | LEU | C | 75 | 9294 | 9342 | 9341 | −3618 | −1242 | 206 | C |
| ATOM | 562 | CG | LEU | C | 75 | 76.727 | 24.270 | 5.358 | 1.00 | 79.27 | | C |
| ANISOU | 562 | CG | LEU | C | 75 | 9988 | 9809 | 10322 | −3652 | −1207 | 216 | C |
| ATOM | 563 | CD1 | LEU | C | 75 | 75.531 | 25.189 | 5.242 | 1.00 | 79.66 | | C |
| ANISOU | 563 | CD1 | LEU | C | 75 | 10148 | 9763 | 10356 | −3600 | −1206 | 45 | C |
| ATOM | 564 | CD2 | LEU | C | 75 | 77.992 | 25.013 | 5.746 | 1.00 | 83.94 | | C |
| ANISOU | 564 | CD2 | LEU | C | 75 | 10446 | 10330 | 11119 | −3771 | −1329 | 179 | C |
| ATOM | 565 | N | HIS | C | 76 | 76.530 | 19.491 | 6.434 | 1.00 | 70.95 | | N |
| ANISOU | 565 | N | HIS | C | 76 | 9076 | 9305 | 8576 | −3512 | −1153 | 593 | N |
| ATOM | 566 | CA | HIS | C | 76 | 76.013 | 18.310 | 7.139 | 1.00 | 70.67 | | C |
| ANISOU | 566 | CA | HIS | C | 76 | 9158 | 9422 | 8270 | −3510 | −1179 | 646 | C |
| ATOM | 567 | C | HIS | C | 76 | 76.815 | 17.938 | 8.419 | 1.00 | 80.32 | | C |
| ANISOU | 567 | C | HIS | C | 76 | 10348 | 10783 | 9386 | −3538 | −1357 | 636 | C |
| ATOM | 568 | O | HIS | C | 76 | 76.270 | 17.249 | 9.294 | 1.00 | 80.58 | | O |
| ANISOU | 568 | O | HIS | C | 76 | 10490 | 10966 | 9162 | −3573 | −1404 | 644 | O |
| ATOM | 569 | CB | HIS | C | 76 | 75.891 | 17.105 | 6.177 | 1.00 | 69.19 | | C |
| ANISOU | 569 | CB | HIS | C | 76 | 9059 | 9174 | 8056 | −3442 | −1072 | 844 | C |
| ATOM | 570 | CG | HIS | C | 76 | 77.161 | 16.325 | 5.945 | 1.00 | 72.54 | | C |
| ANISOU | 570 | CG | HIS | C | 76 | 9432 | 9565 | 8566 | −3370 | −1136 | 994 | C |
| ATOM | 571 | CD2 | HIS | C | 76 | 77.604 | 15.192 | 6.543 | 1.00 | 74.37 | | C |
| ANISOU | 571 | CD2 | HIS | C | 76 | 9737 | 9859 | 8662 | −3335 | −1245 | 1095 | C |
| ATOM | 572 | ND1 | HIS | C | 76 | 78.067 | 16.689 | 4.962 | 1.00 | 73.93 | | N |
| ANISOU | 572 | ND1 | HIS | C | 76 | 9470 | 9641 | 8978 | −3316 | −1090 | 1049 | N |
| ATOM | 573 | CE1 | HIS | C | 76 | 79.042 | 15.797 | 5.019 | 1.00 | 73.65 | | C |
| ANISOU | 573 | CE1 | HIS | C | 76 | 9402 | 9636 | 8946 | −3235 | −1172 | 1153 | C |
| ATOM | 574 | NE2 | HIS | C | 76 | 78.804 | 14.870 | 5.947 | 1.00 | 74.38 | | N |
| ANISOU | 574 | NE2 | HIS | C | 76 | 9632 | 9804 | 8824 | −3231 | −1277 | 1187 | N |
| ATOM | 575 | N | ASP | C | 77 | 78.095 | 18.404 | 8.518 | 1.00 | 80.26 | | N |
| ANISOU | 575 | N | ASP | C | 77 | 10188 | 10741 | 9566 | −3532 | −1455 | 621 | N |
| ATOM | 576 | CA | ASP | C | 77 | 79.006 | 18.133 | 9.635 | 1.00 | 82.83 | | C |
| ANISOU | 576 | CA | ASP | C | 77 | 10452 | 11198 | 9821 | −3536 | −1637 | 601 | C |
| ATOM | 577 | C | ASP | C | 77 | 79.464 | 19.357 | 10.426 | 1.00 | 92.31 | | C |
| ANISOU | 577 | C | ASP | C | 77 | 11517 | 12447 | 11111 | −3608 | −1760 | 393 | C |
| ATOM | 578 | O | ASP | C | 77 | 80.280 | 19.197 | 11.336 | 1.00 | 94.44 | | O |
| ANISOU | 578 | O | ASP | C | 77 | 11716 | 12841 | 11325 | −3607 | −1920 | 359 | O |
| ATOM | 579 | CB | ASP | C | 77 | 80.238 | 17.353 | 9.153 | 1.00 | 84.89 | | C |
| ANISOU | 579 | CB | ASP | C | 77 | 10634 | 11424 | 10196 | −3444 | −1676 | 759 | C |
| ATOM | 580 | CG | ASP | C | 77 | 79.986 | 15.913 | 8.768 | 1.00 | 97.49 | | C |
| ANISOU | 580 | CG | ASP | C | 77 | 12384 | 13000 | 11659 | −3351 | −1641 | 949 | C |
| ATOM | 581 | OD1 | ASP | C | 77 | 79.069 | 15.279 | 9.361 | 1.00 | 98.01 | | O |
| ANISOU | 581 | OD1 | ASP | C | 77 | 12629 | 13130 | 11478 | −3384 | −1652 | 981 | O |
| ATOM | 582 | OD2 | ASP | C | 77 | 80.701 | 15.409 | 7.880 | 1.00 | 105.20 | | O |
| ANISOU | 582 | OD2 | ASP | C | 77 | 13301 | 13900 | 12769 | −3251 | −1606 | 1060 | O |
| ATOM | 583 | N | THR | C | 78 | 78.970 | 20.571 | 10.105 | 1.00 | 90.94 | | N |
| ANISOU | 583 | N | THR | C | 78 | 11313 | 12168 | 11073 | −3661 | −1706 | 245 | N |
| ATOM | 584 | CA | THR | C | 78 | 79.389 | 21.791 | 10.825 | 1.00 | 93.39 | | C |
| ANISOU | 584 | CA | THR | C | 78 | 11510 | 12486 | 11491 | −3733 | −1839 | 26 | C |
| ATOM | 585 | C | THR | C | 78 | 78.973 | 21.802 | 12.304 | 1.00 | 102.22 | | C |
| ANISOU | 585 | C | THR | C | 78 | 12657 | 13829 | 12354 | −3749 | −1977 | −143 | C |
| ATOM | 586 | O | THR | C | 78 | 79.719 | 22.306 | 13.141 | 1.00 | 103.50 | | O |
| ANISOU | 586 | O | THR | C | 78 | 12706 | 14069 | 12551 | −3785 | −2137 | −273 | O |
| ATOM | 587 | CB | THR | C | 78 | 78.927 | 23.069 | 10.112 | 1.00 | 94.52 | | C |
| ANISOU | 587 | CB | THR | C | 78 | 11653 | 12427 | 11834 | −3773 | −1772 | −91 | C |
| ATOM | 588 | CG2 | THR | C | 78 | 79.659 | 23.314 | 8.803 | 1.00 | 90.35 | | C |
| ANISOU | 588 | CG2 | THR | C | 78 | 11053 | 11695 | 11581 | −3798 | −1675 | 60 | C |
| ATOM | 589 | OG1 | THR | C | 78 | 77.516 | 23.013 | 9.908 | 1.00 | 91.27 | | O |
| ANISOU | 589 | OG1 | THR | C | 78 | 11384 | 12027 | 11268 | −3725 | −1669 | −133 | O |
| ATOM | 590 | N | LEU | C | 79 | 77.800 | 21.224 | 12.614 | 1.00 | 100.87 | | N |
| ANISOU | 590 | N | LEU | C | 79 | 12626 | 13786 | 11915 | −3731 | −1914 | −139 | N |
| ATOM | 591 | CA | LEU | C | 79 | 77.195 | 21.183 | 13.946 | 1.00 | 102.98 | | C |
| ANISOU | 591 | CA | LEU | C | 79 | 12931 | 14312 | 11887 | −3758 | −2010 | −288 | C |
| ATOM | 592 | C | LEU | C | 79 | 77.956 | 20.405 | 15.013 | 1.00 | 112.54 | | C |
| ANISOU | 592 | C | LEU | C | 79 | 14130 | 15712 | 12919 | −3760 | −2162 | −214 | C |
| ATOM | 593 | O | LEU | C | 79 | 78.376 | 21.005 | 16.006 | 1.00 | 113.74 | | O |
| ANISOU | 593 | O | LEU | C | 79 | 14187 | 15993 | 13037 | −3780 | −2318 | −394 | O |
| ATOM | 594 | CB | LEU | C | 79 | 75.764 | 20.640 | 13.852 | 1.00 | 101.51 | | C |
| ANISOU | 594 | CB | LEU | C | 79 | 12885 | 14233 | 11450 | −3765 | −1877 | −264 | C |
| ATOM | 595 | CG | LEU | C | 79 | 74.745 | 21.536 | 13.191 | 1.00 | 104.74 | | C |
| ANISOU | 595 | CG | LEU | C | 79 | 13304 | 14543 | 11949 | −3741 | −1764 | −414 | C |
| ATOM | 596 | CD1 | LEU | C | 79 | 73.524 | 20.746 | 12.808 | 1.00 | 103.27 | | C |
| ANISOU | 596 | CD1 | LEU | C | 79 | 13238 | 14446 | 11554 | −3748 | −1609 | −324 | C |
| ATOM | 597 | CD2 | LEU | C | 79 | 74.374 | 22.696 | 14.096 | 1.00 | 108.93 | | C |
| ANISOU | 597 | CD2 | LEU | C | 79 | 13764 | 15186 | 12436 | −3741 | −1873 | −741 | C |
| ATOM | 598 | N | GLU | C | 80 | 78.081 | 19.069 | 14.821 | 1.00 | 112.43 | | N |
| ANISOU | 598 | N | GLU | C | 80 | 14226 | 15709 | 12781 | −3730 | −2128 | 42 | N |
| ATOM | 599 | CA | GLU | C | 80 | 78.640 | 18.047 | 15.724 | 1.00 | 115.74 | | C |
| ANISOU | 599 | CA | GLU | C | 80 | 14703 | 16283 | 12988 | −3709 | −2267 | 175 | C |
| ATOM | 600 | C | GLU | C | 80 | 79.638 | 18.579 | 16.821 | 1.00 | 125.99 | | C |
| ANISOU | 600 | C | GLU | C | 80 | 15859 | 17734 | 14277 | −3703 | −2481 | 20 | C |
| ATOM | 601 | O | GLU | C | 80 | 79.192 | 18.847 | 17.948 | 1.00 | 127.04 | | O |
| ANISOU | 601 | O | GLU | C | 80 | 16007 | 18095 | 14168 | −3751 | −2564 | −125 | O |
| ATOM | 602 | CB | GLU | C | 80 | 79.239 | 16.834 | 14.955 | 1.00 | 116.41 | | C |
| ANISOU | 602 | CB | GLU | C | 80 | 14861 | 16223 | 13148 | −3624 | −2244 | 448 | C |
| ATOM | 603 | CG | GLU | C | 80 | 80.124 | 17.141 | 13.753 | 1.00 | 125.51 | | C |
| ANISOU | 603 | CG | GLU | C | 80 | 15867 | 17170 | 14652 | −3558 | −2195 | 479 | C |
| ATOM | 604 | CD | GLU | C | 80 | 81.444 | 16.392 | 13.667 | 1.00 | 141.12 | | C |
| ANISOU | 604 | CD | GLU | C | 80 | 17779 | 19133 | 16706 | −3445 | −2314 | 613 | C |
| ATOM | 605 | OE1 | GLU | C | 80 | 81.500 | 15.219 | 14.104 | 1.00 | 139.94 | | O |
| ANISOU | 605 | OE1 | GLU | C | 80 | 17779 | 19038 | 16354 | −3381 | −2398 | 769 | O |
| ATOM | 606 | OE2 | GLU | C | 80 | 82.420 | 16.973 | 13.137 | 1.00 | 126.05 | | O |
| ANISOU | 606 | OE2 | GLU | C | 80 | 15673 | 17160 | 15059 | −3422 | −2326 | 562 | O |
| ATOM | 607 | N | ASP | C | 81 | 80.952 | 18.709 | 16.509 | 1.00 | 125.55 | | N |
| ANISOU | 607 | N | ASP | C | 81 | 15654 | 17585 | 14463 | −3648 | −2569 | 40 | N |
| ATOM | 608 | CA | ASP | C | 81 | 81.981 | 19.133 | 17.475 | 1.00 | 128.64 | | C |
| ANISOU | 608 | CA | ASP | C | 81 | 15893 | 18125 | 14858 | −3640 | −2779 | −104 | C |
| ATOM | 609 | C | ASP | C | 81 | 82.358 | 20.622 | 17.377 | 1.00 | 133.55 | | C |
| ANISOU | 609 | C | ASP | C | 81 | 16322 | 18669 | 15752 | −3709 | −2808 | −360 | C |
| ATOM | 610 | O | ASP | C | 81 | 82.735 | 21.216 | 18.396 | 1.00 | 135.42 | | O |
| ANISOU | 610 | O | ASP | C | 81 | 16458 | 19062 | 15933 | −3736 | −2971 | −566 | O |
| ATOM | 611 | CB | ASP | C | 81 | 83.236 | 18.236 | 17.377 | 1.00 | 131.69 | | C |
| ANISOU | 611 | CB | ASP | C | 81 | 16229 | 18519 | 15289 | −3530 | −2895 | 65 | C |
| ATOM | 612 | CG | ASP | C | 81 | 82.969 | 16.757 | 17.613 | 1.00 | 142.98 | | C |
| ANISOU | 612 | CG | ASP | C | 81 | 17880 | 19995 | 16449 | −3453 | −2918 | 314 | C |
| ATOM | 613 | OD1 | ASP | C | 81 | 82.767 | 16.368 | 18.789 | 1.00 | 145.09 | | O |
| ANISOU | 613 | OD1 | ASP | C | 81 | 18251 | 20465 | 16410 | −3465 | −3041 | 316 | O |
| ATOM | 614 | OD2 | ASP | C | 81 | 82.973 | 15.986 | 16.624 | 1.00 | 147.43 | | O |
| ANISOU | 614 | OD2 | ASP | C | 81 | 18522 | 20390 | 17102 | −3385 | −2819 | 507 | O |
| ATOM | 615 | N | CYS | C | 82 | 82.261 | 21.212 | 16.157 | 1.00 | 128.08 | | N |
| ANISOU | 615 | N | CYS | C | 82 | 15590 | 17731 | 15346 | −3742 | −2659 | −343 | N |
| ATOM | 616 | CA | CYS | C | 82 | 82.570 | 22.623 | 15.887 | 1.00 | 128.30 | | C |
| ANISOU | 616 | CA | CYS | C | 82 | 15475 | 17615 | 15658 | −3831 | −2676 | −545 | C |
| ATOM | 617 | C | CYS | C | 82 | 81.519 | 23.543 | 16.513 | 1.00 | 131.12 | | C |
| ANISOU | 617 | C | CYS | C | 82 | 15894 | 18008 | 15919 | −3871 | −2694 | −797 | C |
| ATOM | 618 | O | CYS | C | 82 | 80.357 | 23.151 | 16.679 | 1.00 | 129.14 | | O |
| ANISOU | 618 | O | CYS | C | 82 | 15791 | 17841 | 15435 | −3840 | −2608 | −780 | O |
| ATOM | 619 | CB | CYS | C | 82 | 82.740 | 22.905 | 14.391 | 1.00 | 127.34 | | C |
| ANISOU | 619 | CB | CYS | C | 82 | 15324 | 17230 | 15830 | −3859 | −2511 | −416 | C |
| ATOM | 620 | SG | CYS | C | 82 | 82.942 | 21.432 | 13.345 | 1.00 | 129.40 | | S |
| ANISOU | 620 | SG | CYS | C | 82 | 15661 | 17452 | 16055 | −3749 | −2368 | −81 | S |
| ATOM | 621 | N | GLY | C | 83 | 81.949 | 24.757 | 16.851 | 1.00 | 128.87 | | N |
| ANISOU | 621 | N | GLY | C | 83 | 15487 | 17668 | 15811 | −3940 | −2814 | −1042 | N |
| ATOM | 622 | CA | GLY | C | 83 | 81.115 | 25.785 | 17.463 | 1.00 | 129.10 | | C |
| ANISOU | 622 | CA | GLY | C | 83 | 15549 | 17717 | 15788 | −3956 | −2874 | −1337 | C |
| ATOM | 623 | C | GLY | C | 83 | 80.133 | 26.394 | 16.487 | 1.00 | 129.36 | | C |
| ANISOU | 623 | C | GLY | C | 83 | 15688 | 17510 | 15952 | −3951 | −2725 | −1356 | C |
| ATOM | 624 | O | GLY | C | 83 | 80.255 | 27.567 | 16.111 | 1.00 | 129.89 | | O |
| ANISOU | 624 | O | GLY | C | 83 | 15725 | 17349 | 16279 | −4004 | −2767 | −1512 | O |
| ATOM | 625 | N | VAL | C | 84 | 79.167 | 25.582 | 16.050 | 1.00 | 121.78 | | N |
| ANISOU | 625 | N | VAL | C | 84 | 14863 | 16592 | 14815 | −3889 | −2561 | −1194 | N |
| ATOM | 626 | CA | VAL | C | 84 | 78.139 | 26.008 | 15.115 | 1.00 | 119.12 | | C |
| ANISOU | 626 | CA | VAL | C | 84 | 14631 | 16068 | 14560 | −3857 | −2416 | −1202 | C |
| ATOM | 627 | C | VAL | C | 84 | 76.772 | 25.756 | 15.740 | 1.00 | 120.34 | | C |
| ANISOU | 627 | C | VAL | C | 84 | 14878 | 16458 | 14387 | −3795 | −2379 | −1332 | C |
| ATOM | 628 | O | VAL | C | 84 | 76.432 | 24.617 | 16.081 | 1.00 | 119.05 | | O |
| ANISOU | 628 | O | VAL | C | 84 | 14774 | 16515 | 13946 | −3788 | −2318 | −1184 | O |
| ATOM | 629 | CB | VAL | C | 84 | 78.291 | 25.379 | 13.698 | 1.00 | 120.56 | | C |
| ANISOU | 629 | CB | VAL | C | 84 | 14863 | 16058 | 14888 | −3851 | −2233 | −895 | C |
| ATOM | 630 | CG1 | VAL | C | 84 | 77.209 | 25.885 | 12.747 | 1.00 | 118.98 | | C |
| ANISOU | 630 | CG1 | VAL | C | 84 | 14769 | 15673 | 14762 | −3806 | −2099 | −919 | C |
| ATOM | 631 | CG2 | VAL | C | 84 | 79.675 | 25.651 | 13.116 | 1.00 | 120.94 | | C |
| ANISOU | 631 | CG2 | VAL | C | 84 | 14785 | 15933 | 15235 | −3925 | −2264 | −783 | C |
| ATOM | 632 | N | ALA | C | 85 | 76.018 | 26.845 | 15.926 | 1.00 | 115.86 | | N |
| ANISOU | 632 | N | ALA | C | 85 | 14320 | 15852 | 13850 | −3754 | −2428 | −1620 | N |
| ATOM | 633 | CA | ALA | C | 85 | 74.669 | 26.813 | 16.463 | 1.00 | 114.90 | | C |
| ANISOU | 633 | CA | ALA | C | 85 | 14248 | 15974 | 13433 | −3686 | −2394 | −1803 | C |
| ATOM | 634 | C | ALA | C | 85 | 73.731 | 26.535 | 15.296 | 1.00 | 114.97 | | C |
| ANISOU | 634 | C | ALA | C | 85 | 14361 | 15860 | 13464 | −3635 | −2199 | −1669 | C |
| ATOM | 635 | O | ALA | C | 85 | 73.896 | 27.160 | 14.248 | 1.00 | 113.16 | | O |
| ANISOU | 635 | O | ALA | C | 85 | 14162 | 15307 | 13528 | −3614 | −2162 | −1626 | O |
| ATOM | 636 | CB | ALA | C | 85 | 74.331 | 28.157 | 17.088 | 1.00 | 117.78 | | C |
| ANISOU | 636 | CB | ALA | C | 85 | 14567 | 16335 | 13850 | −3628 | −2551 | −2201 | C |
| ATOM | 637 | N | PRO | C | 86 | 72.730 | 25.635 | 15.422 | 1.00 | 110.63 | | N |
| ANISOU | 637 | N | PRO | C | 86 | 13867 | 15561 | 12606 | −3625 | −2074 | −1601 | N |
| ATOM | 638 | CA | PRO | C | 86 | 71.808 | 25.418 | 14.288 | 1.00 | 108.68 | | C |
| ANISOU | 638 | CA | PRO | C | 86 | 13704 | 15205 | 12385 | −3573 | −1897 | −1501 | C |
| ATOM | 639 | C | PRO | C | 86 | 71.082 | 26.699 | 13.847 | 1.00 | 113.48 | | C |
| ANISOU | 639 | C | PRO | C | 86 | 14320 | 15651 | 13146 | −3457 | −1923 | −1763 | C |
| ATOM | 640 | O | PRO | C | 86 | 70.522 | 26.748 | 12.753 | 1.00 | 111.77 | | O |
| ANISOU | 640 | O | PRO | C | 86 | 14170 | 15257 | 13040 | −3397 | −1805 | −1676 | O |
| ATOM | 641 | CB | PRO | C | 86 | 70.857 | 24.319 | 14.782 | 1.00 | 109.97 | | C |
| ANISOU | 641 | CB | PRO | C | 86 | 13902 | 15722 | 12158 | −3615 | −1792 | −1445 | C |
| ATOM | 642 | CG | PRO | C | 86 | 71.021 | 24.268 | 16.245 | 1.00 | 116.42 | | C |
| ANISOU | 642 | CG | PRO | C | 86 | 14658 | 16855 | 12723 | −3665 | −1923 | −1589 | C |
| ATOM | 643 | CD | PRO | C | 86 | 72.378 | 24.794 | 16.584 | 1.00 | 113.09 | | C |
| ANISOU | 643 | CD | PRO | C | 86 | 14172 | 16271 | 12527 | −3677 | −2089 | −1606 | C |
| ATOM | 644 | N | GLU | C | 87 | 71.154 | 27.751 | 14.681 | 1.00 | 112.30 | | N |
| ANISOU | 644 | N | GLU | C | 87 | 14111 | 15541 | 13016 | −3412 | −2098 | −2087 | N |
| ATOM | 645 | CA | GLU | C | 87 | 70.581 | 29.061 | 14.409 | 1.00 | 113.40 | | C |
| ANISOU | 645 | CA | GLU | C | 87 | 14274 | 15496 | 13315 | −3280 | −2179 | −2372 | C |
| ATOM | 646 | C | GLU | C | 87 | 71.504 | 29.838 | 13.467 | 1.00 | 117.08 | | C |
| ANISOU | 646 | C | GLU | C | 87 | 14791 | 15494 | 14200 | −3308 | −2231 | −2260 | C |
| ATOM | 647 | O | GLU | C | 87 | 70.995 | 30.515 | 12.575 | 1.00 | 116.60 | | O |
| ANISOU | 647 | O | GLU | C | 87 | 14816 | 15180 | 14306 | −3213 | −2210 | −2297 | O |
| ATOM | 648 | CB | GLU | C | 87 | 70.346 | 29.841 | 15.712 | 1.00 | 117.76 | | C |
| ANISOU | 648 | CB | GLU | C | 87 | 14747 | 16269 | 13728 | −3217 | −2364 | −2777 | C |
| ATOM | 649 | CG | GLU | C | 87 | 69.295 | 29.228 | 16.631 | 1.00 | 131.66 | | C |
| ANISOU | 649 | CG | GLU | C | 87 | 16442 | 18535 | 15046 | −3193 | −2308 | −2923 | C |
| ATOM | 650 | CD | GLU | C | 87 | 69.755 | 28.158 | 17.609 | 1.00 | 156.76 | | C |
| ANISOU | 650 | CD | GLU | C | 87 | 19565 | 22045 | 17952 | −3335 | −2306 | −2785 | C |
| ATOM | 651 | OE1 | GLU | C | 87 | 70.981 | 27.926 | 17.724 | 1.00 | 151.42 | | O |
| ANISOU | 651 | OE1 | GLU | C | 87 | 18879 | 21225 | 17428 | −3427 | −2381 | −2623 | O |
| ATOM | 652 | OE2 | GLU | C | 87 | 68.880 | 27.559 | 18.275 | 1.00 | 152.64 | | O |
| ANISOU | 652 | OE2 | GLU | C | 87 | 19006 | 21939 | 17051 | −3357 | −2234 | −2842 | O |
| ATOM | 653 | N | GLU | C | 88 | 72.859 | 29.712 | 13.648 | 1.00 | 113.76 | | N |
| ANISOU | 653 | N | GLU | C | 88 | 14314 | 14975 | 13934 | −3443 | −2296 | −2113 | N |
| ATOM | 654 | CA | GLU | C | 88 | 73.935 | 30.346 | 12.844 | 1.00 | 113.80 | | C |
| ANISOU | 654 | CA | GLU | C | 88 | 14332 | 14588 | 14318 | −3531 | −2338 | −1977 | C |
| ATOM | 655 | C | GLU | C | 88 | 73.740 | 30.047 | 11.348 | 1.00 | 114.99 | | C |
| ANISOU | 655 | C | GLU | C | 88 | 14573 | 14517 | 14602 | −3519 | −2153 | −1689 | C |
| ATOM | 656 | O | GLU | C | 88 | 73.961 | 30.917 | 10.499 | 1.00 | 114.92 | | O |
| ANISOU | 656 | O | GLU | C | 88 | 14630 | 14164 | 14870 | −3530 | −2173 | −1654 | O |
| ATOM | 657 | CB | GLU | C | 88 | 75.326 | 29.826 | 13.292 | 1.00 | 115.62 | | C |
| ANISOU | 657 | CB | GLU | C | 88 | 14447 | 14886 | 14596 | −3676 | −2391 | −1829 | C |
| ATOM | 658 | CG | GLU | C | 88 | 76.313 | 30.876 | 13.793 | 1.00 | 129.04 | | C |
| ANISOU | 658 | CG | GLU | C | 88 | 16079 | 16389 | 16561 | −3774 | −2583 | −1980 | C |
| ATOM | 659 | CD | GLU | C | 88 | 77.547 | 30.342 | 14.511 | 1.00 | 145.62 | | C |
| ANISOU | 659 | CD | GLU | C | 88 | 18034 | 18656 | 18641 | −3887 | −2668 | −1919 | C |
| ATOM | 660 | OE1 | GLU | C | 88 | 77.384 | 29.580 | 15.492 | 1.00 | 135.52 | | O |
| ANISOU | 660 | OE1 | GLU | C | 88 | 16708 | 17721 | 17064 | −3850 | −2699 | −1973 | O |
| ATOM | 661 | OE2 | GLU | C | 88 | 78.677 | 30.720 | 14.121 | 1.00 | 135.29 | | O |
| ANISOU | 661 | OE2 | GLU | C | 88 | 16653 | 17148 | 17604 | −4017 | −2712 | −1826 | O |
| ATOM | 662 | N | LEU | C | 89 | 73.320 | 28.797 | 11.051 | 1.00 | 108.61 | | N |
| ANISOU | 662 | N | LEU | C | 89 | 13774 | 13911 | 13582 | −3502 | −1982 | −1483 | N |
| ATOM | 663 | CA | LEU | C | 89 | 73.033 | 28.268 | 9.719 | 1.00 | 105.38 | | C |
| ANISOU | 663 | CA | LEU | C | 89 | 13438 | 13373 | 13229 | −3475 | −1794 | −1221 | C |
| ATOM | 664 | C | LEU | C | 89 | 71.693 | 28.816 | 9.246 | 1.00 | 108.76 | | C |
| ANISOU | 664 | C | LEU | C | 89 | 13960 | 13754 | 13609 | −3327 | −1756 | −1377 | C |
| ATOM | 665 | O | LEU | C | 89 | 71.586 | 29.213 | 8.092 | 1.00 | 107.45 | | O |
| ANISOU | 665 | O | LEU | C | 89 | 13873 | 13326 | 13626 | −3288 | −1691 | −1262 | O |
| ATOM | 666 | CB | LEU | C | 89 | 72.972 | 26.721 | 9.741 | 1.00 | 103.12 | | C |
| ANISOU | 666 | CB | LEU | C | 89 | 13138 | 13334 | 12709 | −3503 | −1657 | −996 | C |
| ATOM | 667 | CG | LEU | C | 89 | 74.212 | 25.948 | 10.196 | 1.00 | 107.09 | | C |
| ANISOU | 667 | CG | LEU | C | 89 | 13559 | 13915 | 13214 | −3607 | −1695 | −823 | C |
| ATOM | 668 | CD1 | LEU | C | 89 | 73.819 | 24.661 | 10.871 | 1.00 | 106.20 | | C |
| ANISOU | 668 | CD1 | LEU | C | 89 | 13464 | 14107 | 12780 | −3615 | −1648 | −744 | C |
| ATOM | 669 | CD2 | LEU | C | 89 | 75.119 | 25.639 | 9.039 | 1.00 | 107.98 | | C |
| ANISOU | 669 | CD2 | LEU | C | 89 | 13656 | 13819 | 13552 | −3646 | −1601 | −546 | C |
| ATOM | 670 | N | GLU | C | 90 | 70.668 | 28.819 | 10.133 | 1.00 | 106.28 | | N |
| ANISOU | 670 | N | GLU | C | 90 | 13628 | 13720 | 13033 | −3240 | −1796 | −1641 | N |
| ATOM | 671 | CA | GLU | C | 90 | 69.321 | 29.300 | 9.820 | 1.00 | 106.35 | | C |
| ANISOU | 671 | CA | GLU | C | 90 | 13694 | 13759 | 12956 | −3073 | −1773 | −1841 | C |
| ATOM | 672 | C | GLU | C | 90 | 69.330 | 30.779 | 9.448 | 1.00 | 111.66 | | C |
| ANISOU | 672 | C | GLU | C | 90 | 14445 | 14082 | 13899 | −2975 | −1920 | −2022 | C |
| ATOM | 673 | O | GLU | C | 90 | 68.631 | 31.183 | 8.518 | 1.00 | 110.91 | | O |
| ANISOU | 673 | O | GLU | C | 90 | 14443 | 13827 | 13872 | −2845 | −1878 | −2027 | O |
| ATOM | 674 | CB | GLU | C | 90 | 68.355 | 29.040 | 10.983 | 1.00 | 108.63 | | C |
| ANISOU | 674 | CB | GLU | C | 90 | 13908 | 14474 | 12894 | −3020 | −1797 | −2114 | C |
| ATOM | 675 | CG | GLU | C | 90 | 66.925 | 28.826 | 10.515 | 1.00 | 119.03 | | C |
| ANISOU | 675 | CG | GLU | C | 90 | 15245 | 15948 | 14034 | −2884 | −1686 | −2215 | C |
| ATOM | 676 | CD | GLU | C | 90 | 65.862 | 29.795 | 10.997 | 1.00 | 142.67 | | C |
| ANISOU | 676 | CD | GLU | C | 90 | 18193 | 19131 | 16883 | −2709 | −1797 | −2640 | C |
| ATOM | 677 | OE1 | GLU | C | 90 | 64.996 | 30.175 | 10.174 | 1.00 | 122.55 | | O |
| ANISOU | 677 | OE1 | GLU | C | 90 | 15683 | 16552 | 14330 | −2546 | −1758 | −2745 | O |
| ATOM | 678 | OE2 | GLU | C | 90 | 65.867 | 30.142 | 12.201 | 1.00 | 146.29 | | O |
| ANISOU | 678 | OE2 | GLU | C | 90 | 18570 | 19796 | 17219 | −2720 | −1927 | −2882 | O |
| ATOM | 679 | N | ARG | C | 91 | 70.147 | 31.568 | 10.155 | 1.00 | 109.87 | | N |
| ANISOU | 679 | N | ARG | C | 91 | 14190 | 13727 | 13827 | −3038 | −2102 | −2162 | N |
| ATOM | 680 | CA | ARG | C | 91 | 70.315 | 33.002 | 9.943 | 1.00 | 112.00 | | C |
| ANISOU | 680 | CA | ARG | C | 91 | 14553 | 13625 | 14376 | −2981 | −2280 | −2335 | C |
| ATOM | 681 | C | ARG | C | 91 | 71.114 | 33.296 | 8.642 | 1.00 | 116.00 | | C |
| ANISOU | 681 | C | ARG | C | 91 | 15156 | 13718 | 15201 | −3085 | −2222 | −2016 | C |
| ATOM | 682 | O | ARG | C | 91 | 70.936 | 34.365 | 8.044 | 1.00 | 117.12 | | O |
| ANISOU | 682 | O | ARG | C | 91 | 15435 | 13514 | 15552 | −3015 | −2315 | −2075 | O |
| ATOM | 683 | CB | ARG | C | 91 | 70.992 | 33.608 | 11.191 | 1.00 | 113.79 | | C |
| ANISOU | 683 | CB | ARG | C | 91 | 14703 | 13886 | 14647 | −3049 | −2491 | −2585 | C |
| ATOM | 684 | CG | ARG | C | 91 | 71.246 | 35.118 | 11.179 | 1.00 | 123.03 | | C |
| ANISOU | 684 | CG | ARG | C | 91 | 15975 | 14661 | 16112 | −3013 | −2717 | −2806 | C |
| ATOM | 685 | CD | ARG | C | 91 | 72.206 | 35.541 | 12.284 | 1.00 | 128.20 | | C |
| ANISOU | 685 | CD | ARG | C | 91 | 16537 | 15330 | 16845 | −3139 | −2905 | −2981 | C |
| ATOM | 686 | NE | ARG | C | 91 | 73.383 | 34.671 | 12.379 | 1.00 | 130.62 | | N |
| ANISOU | 686 | NE | ARG | C | 91 | 16724 | 15738 | 17167 | −3357 | −2815 | −2695 | N |
| ATOM | 687 | CZ | ARG | C | 91 | 74.497 | 34.819 | 11.666 | 1.00 | 141.21 | | C |
| ANISOU | 687 | CZ | ARG | C | 91 | 18074 | 16791 | 18789 | −3541 | −2791 | −2433 | C |
| ATOM | 688 | NH1 | ARG | C | 91 | 74.608 | 35.814 | 10.794 | 1.00 | 130.05 | | N |
| ANISOU | 688 | NH1 | ARG | C | 91 | 16803 | 14945 | 17664 | −3565 | −2845 | −2389 | N |
| ATOM | 689 | NH2 | ARG | C | 91 | 75.507 | 33.973 | 11.818 | 1.00 | 123.24 | | N |
| ANISOU | 689 | NH2 | ARG | C | 91 | 15665 | 14667 | 16493 | −3700 | −2718 | −2212 | N |
| ATOM | 690 | N | ARG | C | 92 | 71.960 | 32.339 | 8.196 | 1.00 | 110.70 | | N |
| ANISOU | 690 | N | ARG | C | 92 | 14418 | 13095 | 14549 | −3242 | −2073 | −1680 | N |
| ATOM | 691 | CA | ARG | C | 92 | 72.820 | 32.505 | 7.015 | 1.00 | 109.91 | | C |
| ANISOU | 691 | CA | ARG | C | 92 | 14364 | 12684 | 14711 | −3366 | −1998 | −1368 | C |
| ATOM | 692 | C | ARG | C | 92 | 72.361 | 31.804 | 5.722 | 1.00 | 109.97 | | C |
| ANISOU | 692 | C | ARG | C | 92 | 14430 | 12681 | 14671 | −3307 | −1783 | −1095 | C |
| ATOM | 693 | O | ARG | C | 92 | 72.736 | 32.262 | 4.638 | 1.00 | 109.49 | | O |
| ANISOU | 693 | O | ARG | C | 92 | 14449 | 12332 | 14819 | −3355 | −1741 | −899 | O |
| ATOM | 694 | CB | ARG | C | 92 | 74.259 | 32.057 | 7.342 | 1.00 | 110.25 | | C |
| ANISOU | 694 | CB | ARG | C | 92 | 14268 | 12783 | 14841 | −3574 | −1998 | −1204 | C |
| ATOM | 695 | CG | ARG | C | 92 | 75.087 | 33.077 | 8.122 | 1.00 | 121.32 | | C |
| ANISOU | 695 | CG | ARG | C | 92 | 15631 | 14030 | 16437 | −3695 | −2212 | −1390 | C |
| ATOM | 696 | CD | ARG | C | 92 | 76.565 | 32.716 | 8.177 | 1.00 | 127.39 | | C |
| ANISOU | 696 | CD | ARG | C | 92 | 16253 | 14815 | 17336 | −3908 | −2199 | −1196 | C |
| ATOM | 697 | NE | ARG | C | 92 | 77.180 | 32.707 | 6.844 | 1.00 | 133.70 | | N |
| ANISOU | 697 | NE | ARG | C | 92 | 17073 | 15399 | 18326 | −4016 | −2063 | −877 | N |
| ATOM | 698 | CZ | ARG | C | 92 | 78.486 | 32.808 | 6.610 | 1.00 | 146.20 | | C |
| ANISOU | 698 | CZ | ARG | C | 92 | 18545 | 16900 | 20106 | −4224 | −2065 | −722 | C |
| ATOM | 699 | NH1 | ARG | C | 92 | 79.341 | 32.941 | 7.616 | 1.00 | 135.82 | | N |
| ANISOU | 699 | NH1 | ARG | C | 92 | 17091 | 15676 | 18839 | −4343 | −2210 | −860 | N |
| ATOM | 700 | NH2 | ARG | C | 92 | 78.946 | 32.783 | 5.364 | 1.00 | 128.22 | | N |
| ANISOU | 700 | NH2 | ARG | C | 92 | 16280 | 14473 | 17966 | −4317 | −1924 | −436 | N |
| ATOM | 701 | N | PHE | C | 93 | 71.600 | 30.686 | 5.828 | 1.00 | 103.52 | | N |
| ANISOU | 701 | N | PHE | C | 93 | 13572 | 12181 | 13579 | −3224 | −1650 | −1072 | N |
| ATOM | 702 | CA | PHE | C | 93 | 71.206 | 29.854 | 4.677 | 1.00 | 100.53 | | C |
| ANISOU | 702 | CA | PHE | C | 93 | 13229 | 11833 | 13133 | −3176 | −1447 | −826 | C |
| ATOM | 703 | C | PHE | C | 93 | 69.698 | 29.478 | 4.565 | 1.00 | 104.00 | | C |
| ANISOU | 703 | C | PHE | C | 93 | 13709 | 12481 | 13326 | −3001 | −1370 | −959 | C |
| ATOM | 704 | O | PHE | C | 93 | 69.308 | 28.771 | 3.627 | 1.00 | 101.79 | | O |
| ANISOU | 704 | O | PHE | C | 93 | 13456 | 12238 | 12982 | −2958 | −1209 | −779 | O |
| ATOM | 705 | CB | PHE | C | 93 | 72.066 | 28.572 | 4.652 | 1.00 | 99.91 | | C |
| ANISOU | 705 | CB | PHE | C | 93 | 13047 | 11917 | 12996 | −3301 | −1328 | −570 | C |
| ATOM | 706 | CG | PHE | C | 93 | 73.552 | 28.828 | 4.755 | 1.00 | 101.42 | | C |
| ANISOU | 706 | CG | PHE | C | 93 | 13157 | 11973 | 13404 | −3468 | −1392 | −450 | C |
| ATOM | 707 | CD1 | PHE | C | 93 | 74.263 | 29.339 | 3.677 | 1.00 | 104.00 | | C |
| ANISOU | 707 | CD1 | PHE | C | 93 | 13508 | 12034 | 13974 | −3546 | −1348 | −253 | C |
| ATOM | 708 | CD2 | PHE | C | 93 | 74.233 | 28.587 | 5.939 | 1.00 | 103.37 | | C |
| ANISOU | 708 | CD2 | PHE | C | 93 | 13295 | 12382 | 13598 | −3556 | −1499 | −539 | C |
| ATOM | 709 | CE1 | PHE | C | 93 | 75.625 | 29.596 | 3.780 | 1.00 | 105.64 | | C |
| ANISOU | 709 | CE1 | PHE | C | 93 | 13613 | 12154 | 14371 | −3722 | −1402 | −158 | C |
| ATOM | 710 | CE2 | PHE | C | 93 | 75.602 | 28.832 | 6.034 | 1.00 | 106.94 | | C |
| ANISOU | 710 | CE2 | PHE | C | 93 | 13647 | 12739 | 14246 | −3708 | −1563 | −449 | C |
| ATOM | 711 | CZ | PHE | C | 93 | 76.287 | 29.330 | 4.952 | 1.00 | 105.30 | | C |
| ANISOU | 711 | CZ | PHE | C | 93 | 13446 | 12281 | 14281 | −3797 | −1509 | −264 | C |
| ATOM | 712 | N | GLY | C | 94 | 68.877 | 29.973 | 5.484 | 1.00 | 101.92 | | N |
| ANISOU | 712 | N | GLY | C | 94 | 13435 | 12365 | 12925 | −2900 | −1487 | −1286 | N |
| ATOM | 713 | CA | GLY | C | 94 | 67.444 | 29.705 | 5.470 | 1.00 | 101.03 | | C |
| ANISOU | 713 | CA | GLY | C | 94 | 13326 | 12493 | 12569 | −2741 | −1427 | −1457 | C |
| ATOM | 714 | C | GLY | C | 94 | 67.026 | 28.594 | 6.413 | 1.00 | 104.23 | | C |
| ANISOU | 714 | C | GLY | C | 94 | 13623 | 13324 | 12655 | −2802 | −1356 | −1513 | C |
| ATOM | 715 | O | GLY | C | 94 | 67.868 | 27.806 | 6.859 | 1.00 | 103.32 | | O |
| ANISOU | 715 | O | GLY | C | 94 | 13454 | 13298 | 12505 | −2959 | −1327 | −1347 | O |
| ATOM | 716 | N | PRO | C | 95 | 65.715 | 28.509 | 6.749 | 1.00 | 100.77 | | N |
| ANISOU | 716 | N | PRO | C | 95 | 13150 | 13170 | 11968 | −2684 | −1334 | −1749 | N |
| ATOM | 717 | CA | PRO | C | 95 | 65.259 | 27.437 | 7.653 | 1.00 | 99.53 | | C |
| ANISOU | 717 | CA | PRO | C | 95 | 12897 | 13440 | 11480 | −2780 | −1256 | −1785 | C |
| ATOM | 718 | C | PRO | C | 95 | 65.292 | 26.067 | 6.988 | 1.00 | 98.51 | | C |
| ANISOU | 718 | C | PRO | C | 95 | 12787 | 13378 | 11265 | −2888 | −1065 | −1469 | C |
| ATOM | 719 | O | PRO | C | 95 | 65.418 | 25.055 | 7.676 | 1.00 | 97.87 | | O |
| ANISOU | 719 | O | PRO | C | 95 | 12669 | 13534 | 10985 | −3032 | −1016 | −1375 | O |
| ATOM | 720 | CB | PRO | C | 95 | 63.841 | 27.864 | 8.024 | 1.00 | 102.86 | | C |
| ANISOU | 720 | CB | PRO | C | 95 | 13261 | 14137 | 11684 | −2622 | −1284 | −2140 | C |
| ATOM | 721 | CG | PRO | C | 95 | 63.381 | 28.694 | 6.880 | 1.00 | 107.86 | | C |
| ANISOU | 721 | CG | PRO | C | 95 | 13983 | 14493 | 12508 | −2426 | −1304 | −2186 | C |
| ATOM | 722 | CD | PRO | C | 95 | 64.594 | 29.368 | 6.311 | 1.00 | 103.56 | | C |
| ANISOU | 722 | CD | PRO | C | 95 | 13546 | 13484 | 12318 | −2462 | −1380 | −1990 | C |
| ATOM | 723 | N | THR | C | 96 | 65.194 | 26.058 | 5.646 | 1.00 | 91.62 | | N |
| ANISOU | 723 | N | THR | C | 96 | 11987 | 12283 | 10543 | −2814 | −972 | −1307 | N |
| ATOM | 724 | CA | THR | C | 96 | 65.245 | 24.889 | 4.779 | 1.00 | 88.63 | | C |
| ANISOU | 724 | CA | THR | C | 96 | 11638 | 11905 | 10131 | −2880 | −803 | −1022 | C |
| ATOM | 725 | C | THR | C | 96 | 66.552 | 24.128 | 5.007 | 1.00 | 90.10 | | C |
| ANISOU | 725 | C | THR | C | 96 | 11825 | 12009 | 10399 | −3038 | −797 | −759 | C |
| ATOM | 726 | O | THR | C | 96 | 66.507 | 22.950 | 5.347 | 1.00 | 89.11 | | O |
| ANISOU | 726 | O | THR | C | 96 | 11694 | 12068 | 10095 | −3146 | −726 | −637 | O |
| ATOM | 727 | CB | THR | C | 96 | 65.040 | 25.311 | 3.313 | 1.00 | 96.84 | | C |
| ANISOU | 727 | CB | THR | C | 96 | 12753 | 12695 | 11347 | −2745 | −740 | −930 | C |
| ATOM | 728 | CG2 | THR | C | 96 | 63.568 | 25.334 | 2.911 | 1.00 | 94.18 | | C |
| ANISOU | 728 | CG2 | THR | C | 96 | 12404 | 12547 | 10834 | −2602 | −681 | −1117 | C |
| ATOM | 729 | OG1 | THR | C | 96 | 65.626 | 26.604 | 3.102 | 1.00 | 99.95 | | O |
| ANISOU | 729 | OG1 | THR | C | 96 | 13198 | 12776 | 12001 | −2675 | −868 | −975 | O |
| ATOM | 730 | N | VAL | C | 97 | 67.704 | 24.821 | 4.893 | 1.00 | 86.07 | | N |
| ANISOU | 730 | N | VAL | C | 97 | 11321 | 11233 | 10147 | −3056 | −888 | −689 | N |
| ATOM | 731 | CA | VAL | C | 97 | 69.055 | 24.272 | 5.096 | 1.00 | 85.03 | | C |
| ANISOU | 731 | CA | VAL | C | 97 | 11161 | 11028 | 10118 | −3182 | −907 | −474 | C |
| ATOM | 732 | C | VAL | C | 97 | 69.249 | 23.829 | 6.549 | 1.00 | 87.93 | | C |
| ANISOU | 732 | C | VAL | C | 97 | 11473 | 11639 | 10296 | −3279 | −997 | −564 | C |
| ATOM | 733 | O | VAL | C | 97 | 69.829 | 22.772 | 6.777 | 1.00 | 85.84 | | O |
| ANISOU | 733 | O | VAL | C | 97 | 11205 | 11451 | 9959 | −3367 | −971 | −379 | O |
| ATOM | 734 | CB | VAL | C | 97 | 70.161 | 25.245 | 4.585 | 1.00 | 89.91 | | C |
| ANISOU | 734 | CB | VAL | C | 97 | 11776 | 11328 | 11057 | −3195 | −976 | −398 | C |
| ATOM | 735 | CG1 | VAL | C | 97 | 71.567 | 24.797 | 4.993 | 1.00 | 89.95 | | C |
| ANISOU | 735 | CG1 | VAL | C | 97 | 11709 | 11315 | 11153 | −3320 | −1022 | −240 | C |
| ATOM | 736 | CG2 | VAL | C | 97 | 70.075 | 25.426 | 3.070 | 1.00 | 88.74 | | C |
| ANISOU | 736 | CG2 | VAL | C | 97 | 11690 | 10967 | 11058 | −3124 | −862 | −233 | C |
| ATOM | 737 | N | ARG | C | 98 | 68.719 | 24.604 | 7.517 | 1.00 | 86.76 | | N |
| ANISOU | 737 | N | ARG | C | 98 | 11290 | 11627 | 10049 | −3248 | −1110 | −855 | N |
| ATOM | 738 | CA | ARG | C | 98 | 68.780 | 24.269 | 8.943 | 1.00 | 88.18 | | C |
| ANISOU | 738 | CA | ARG | C | 98 | 11413 | 12085 | 10008 | −3332 | −1198 | −968 | C |
| ATOM | 739 | C | ARG | C | 98 | 68.159 | 22.873 | 9.160 | 1.00 | 92.44 | | C |
| ANISOU | 739 | C | ARG | C | 98 | 11977 | 12899 | 10245 | −3414 | −1081 | −844 | C |
| ATOM | 740 | O | ARG | C | 98 | 68.851 | 21.981 | 9.644 | 1.00 | 91.06 | | O |
| ANISOU | 740 | O | ARG | C | 98 | 11814 | 12795 | 9989 | −3520 | −1099 | −673 | O |
| ATOM | 741 | CB | ARG | C | 98 | 68.084 | 25.349 | 9.793 | 1.00 | 89.90 | | C |
| ANISOU | 741 | CB | ARG | C | 98 | 11580 | 12432 | 10144 | −3252 | −1322 | −1337 | C |
| ATOM | 742 | CG | ARG | C | 98 | 68.190 | 25.128 | 11.302 | 1.00 | 100.63 | | C |
| ANISOU | 742 | CG | ARG | C | 98 | 12869 | 14107 | 11259 | −3333 | −1421 | −1477 | C |
| ATOM | 743 | CD | ARG | C | 98 | 66.919 | 25.536 | 12.021 | 1.00 | 110.02 | | C |
| ANISOU | 743 | CD | ARG | C | 98 | 13999 | 15614 | 12188 | −3258 | −1444 | −1813 | C |
| ATOM | 744 | NE | ARG | C | 98 | 66.996 | 25.323 | 13.466 | 1.00 | 118.95 | | N |
| ANISOU | 744 | NE | ARG | C | 98 | 15056 | 17087 | 13052 | −3343 | −1530 | −1943 | N |
| ATOM | 745 | CZ | ARG | C | 98 | 67.247 | 26.275 | 14.362 | 1.00 | 133.93 | | C |
| ANISOU | 745 | CZ | ARG | C | 98 | 16884 | 19041 | 14962 | −3290 | −1703 | −2227 | C |
| ATOM | 746 | NH1 | ARG | C | 98 | 67.455 | 27.527 | 13.971 | 1.00 | 125.19 | | N |
| ANISOU | 746 | NH1 | ARG | C | 98 | 15787 | 17639 | 14140 | −3162 | −1818 | −2411 | N |
| ATOM | 747 | NH2 | ARG | C | 98 | 67.288 | 25.983 | 15.655 | 1.00 | 117.00 | | N |
| ANISOU | 747 | NH2 | ARG | C | 98 | 14670 | 17245 | 12538 | −3369 | −1769 | −2328 | N |
| ATOM | 748 | N | ARG | C | 99 | 66.900 | 22.671 | 8.703 | 1.00 | 90.73 | | N |
| ANISOU | 748 | N | ARG | C | 99 | 11779 | 12812 | 9883 | −3368 | −965 | −914 | N |
| ATOM | 749 | CA | ARG | C | 99 | 66.155 | 21.400 | 8.758 | 1.00 | 90.78 | | C |
| ANISOU | 749 | CA | ARG | C | 99 | 11816 | 13061 | 9614 | −3470 | −841 | −804 | C |
| ATOM | 750 | C | ARG | C | 99 | 66.986 | 20.202 | 8.220 | 1.00 | 92.69 | | C |
| ANISOU | 750 | C | ARG | C | 99 | 12143 | 13151 | 9923 | −3553 | −780 | −458 | C |
| ATOM | 751 | O | ARG | C | 99 | 67.167 | 19.222 | 8.952 | 1.00 | 92.89 | | O |
| ANISOU | 751 | O | ARG | C | 99 | 12209 | 13330 | 9756 | −3683 | −794 | −338 | O |
| ATOM | 752 | CB | ARG | C | 99 | 64.813 | 21.506 | 7.984 | 1.00 | 92.38 | | C |
| ANISOU | 752 | CB | ARG | C | 99 | 12012 | 13357 | 9733 | −3387 | −723 | −926 | C |
| ATOM | 753 | CG | ARG | C | 99 | 63.711 | 22.311 | 8.680 | 1.00 | 108.81 | | C |
| ANISOU | 753 | CG | ARG | C | 99 | 13994 | 15735 | 11612 | −3323 | −764 | −1285 | C |
| ATOM | 754 | CD | ARG | C | 99 | 62.354 | 22.138 | 8.000 | 1.00 | 123.94 | | C |
| ANISOU | 754 | CD | ARG | C | 99 | 15882 | 17834 | 13376 | −3274 | −635 | −1387 | C |
| ATOM | 755 | NE | ARG | C | 99 | 61.668 | 20.912 | 8.437 | 1.00 | 137.89 | | N |
| ANISOU | 755 | NE | ARG | C | 99 | 17648 | 19907 | 14839 | −3474 | −521 | −1281 | N |
| ATOM | 756 | CZ | ARG | C | 99 | 60.412 | 20.585 | 8.129 | 1.00 | 151.10 | | C |
| ANISOU | 756 | CZ | ARG | C | 99 | 19268 | 21839 | 16304 | −3502 | −404 | −1379 | C |
| ATOM | 757 | NH1 | ARG | C | 99 | 59.885 | 19.453 | 8.577 | 1.00 | 132.91 | | N |
| ANISOU | 757 | NH1 | ARG | C | 99 | 16980 | 19786 | 13735 | −3729 | −310 | −1256 | N |
| ATOM | 758 | NH2 | ARG | C | 99 | 59.673 | 21.390 | 7.373 | 1.00 | 140.70 | | N |
| ANISOU | 758 | NH2 | ARG | C | 99 | 17887 | 20532 | 15042 | −3308 | −388 | −1600 | N |
| ATOM | 759 | N | ILE | C | 100 | 67.509 | 20.310 | 6.965 | 1.00 | 86.48 | | N |
| ANISOU | 759 | N | ILE | C | 100 | 11388 | 12069 | 9401 | −3470 | −726 | −305 | N |
| ATOM | 760 | CA | ILE | C | 100 | 68.307 | 19.280 | 6.279 | 1.00 | 84.67 | | C |
| ANISOU | 760 | CA | ILE | C | 100 | 11221 | 11684 | 9265 | −3500 | −671 | −12 | C |
| ATOM | 761 | C | ILE | C | 100 | 69.628 | 18.978 | 6.999 | 1.00 | 90.82 | | C |
| ANISOU | 761 | C | ILE | C | 100 | 11988 | 12418 | 10101 | −3558 | −790 | 109 | C |
| ATOM | 762 | O | ILE | C | 100 | 69.969 | 17.802 | 7.151 | 1.00 | 89.94 | | O |
| ANISOU | 762 | O | ILE | C | 100 | 11944 | 12333 | 9895 | −3618 | −787 | 295 | O |
| ATOM | 763 | CB | ILE | C | 100 | 68.539 | 19.590 | 4.771 | 1.00 | 86.03 | | C |
| ANISOU | 763 | CB | ILE | C | 100 | 11403 | 11598 | 9686 | −3387 | −583 | 92 | C |
| ATOM | 764 | CG1 | ILE | C | 100 | 67.232 | 19.999 | 4.062 | 1.00 | 85.67 | | C |
| ANISOU | 764 | CG1 | ILE | C | 100 | 11367 | 11602 | 9583 | −3302 | −486 | −46 | C |
| ATOM | 765 | CG2 | ILE | C | 100 | 69.177 | 18.385 | 4.063 | 1.00 | 85.78 | | C |
| ANISOU | 765 | CG2 | ILE | C | 100 | 11426 | 11465 | 9700 | −3402 | −518 | 357 | C |
| ATOM | 766 | CD1 | ILE | C | 100 | 67.412 | 20.764 | 2.758 | 1.00 | 90.31 | | C |
| ANISOU | 766 | CD1 | ILE | C | 100 | 11959 | 11941 | 10413 | −3171 | −441 | −7 | C |
| ATOM | 767 | N | VAL | C | 101 | 70.372 | 20.033 | 7.417 | 1.00 | 90.00 | | N |
| ANISOU | 767 | N | VAL | C | 101 | 11804 | 12234 | 10159 | −3533 | −907 | −1 | N |
| ATOM | 768 | CA | VAL | C | 101 | 71.654 | 19.914 | 8.129 | 1.00 | 91.51 | | C |
| ANISOU | 768 | CA | VAL | C | 101 | 11951 | 12404 | 10415 | −3577 | −1037 | 72 | C |
| ATOM | 769 | C | VAL | C | 101 | 71.424 | 19.245 | 9.495 | 1.00 | 99.15 | | C |
| ANISOU | 769 | C | VAL | C | 101 | 12949 | 13641 | 11082 | −3669 | −1115 | 41 | C |
| ATOM | 770 | O | VAL | C | 101 | 72.145 | 18.302 | 9.834 | 1.00 | 99.03 | | O |
| ANISOU | 770 | O | VAL | C | 101 | 12977 | 13642 | 11010 | −3707 | −1168 | 219 | O |
| ATOM | 771 | CB | VAL | C | 101 | 72.419 | 21.271 | 8.209 | 1.00 | 96.30 | | C |
| ANISOU | 771 | CB | VAL | C | 101 | 12458 | 12862 | 11270 | −3552 | −1142 | −60 | C |
| ATOM | 772 | CG1 | VAL | C | 101 | 73.594 | 21.213 | 9.177 | 1.00 | 97.48 | | C |
| ANISOU | 772 | CG1 | VAL | C | 101 | 12536 | 13063 | 11437 | −3607 | −1294 | −49 | C |
| ATOM | 773 | CG2 | VAL | C | 101 | 72.903 | 21.711 | 6.833 | 1.00 | 95.20 | | C |
| ANISOU | 773 | CG2 | VAL | C | 101 | 12304 | 12452 | 11416 | −3499 | −1064 | 57 | C |
| ATOM | 774 | N | GLU | C | 102 | 70.379 | 19.697 | 10.238 | 1.00 | 98.59 | | N |
| ANISOU | 774 | N | GLU | C | 102 | 12862 | 13795 | 10803 | −3698 | −1122 | −182 | N |
| ATOM | 775 | CA | GLU | C | 102 | 69.973 | 19.150 | 11.540 | 1.00 | 100.72 | | C |
| ANISOU | 775 | CA | GLU | C | 102 | 13153 | 14375 | 10740 | −3805 | −1177 | −228 | C |
| ATOM | 776 | C | GLU | C | 102 | 69.515 | 17.702 | 11.402 | 1.00 | 106.09 | | C |
| ANISOU | 776 | C | GLU | C | 102 | 13961 | 15139 | 11210 | −3903 | −1084 | −7 | C |
| ATOM | 777 | O | GLU | C | 102 | 69.785 | 16.900 | 12.290 | 1.00 | 106.81 | | O |
| ANISOU | 777 | O | GLU | C | 102 | 14118 | 15371 | 11096 | −4001 | −1154 | 106 | O |
| ATOM | 778 | CB | GLU | C | 102 | 68.827 | 19.967 | 12.157 | 1.00 | 103.23 | | C |
| ANISOU | 778 | CB | GLU | C | 102 | 13401 | 14942 | 10880 | −3799 | −1178 | −538 | C |
| ATOM | 779 | CG | GLU | C | 102 | 69.251 | 21.210 | 12.916 | 1.00 | 115.00 | | C |
| ANISOU | 779 | CG | GLU | C | 102 | 14786 | 16450 | 12459 | −3741 | −1332 | −792 | C |
| ATOM | 780 | CD | GLU | C | 102 | 68.109 | 21.920 | 13.619 | 1.00 | 136.08 | | C |
| ANISOU | 780 | CD | GLU | C | 102 | 17382 | 19401 | 14921 | −3711 | −1348 | −1125 | C |
| ATOM | 781 | OE1 | GLU | C | 102 | 68.206 | 22.120 | 14.851 | 1.00 | 131.84 | | O |
| ANISOU | 781 | OE1 | GLU | C | 102 | 16788 | 19118 | 14189 | −3753 | −1458 | −1276 | O |
| ATOM | 782 | OE2 | GLU | C | 102 | 67.109 | 22.261 | 12.944 | 1.00 | 129.45 | | O |
| ANISOU | 782 | OE2 | GLU | C | 102 | 16534 | 18553 | 14099 | −3632 | −1255 | −1246 | O |
| ATOM | 783 | N | GLY | C | 103 | 68.809 | 17.400 | 10.310 | 1.00 | 102.93 | | N |
| ANISOU | 783 | N | GLY | C | 103 | 13604 | 14649 | 10856 | −3879 | −938 | 49 | N |
| ATOM | 784 | CA | GLY | C | 103 | 68.294 | 16.070 | 10.001 | 1.00 | 103.32 | | C |
| ANISOU | 784 | CA | GLY | C | 103 | 13781 | 14734 | 10741 | −3977 | −843 | 244 | C |
| ATOM | 785 | C | GLY | C | 103 | 69.382 | 15.023 | 9.873 | 1.00 | 109.37 | | C |
| ANISOU | 785 | C | GLY | C | 103 | 14654 | 15317 | 11584 | −3980 | −908 | 520 | C |
| ATOM | 786 | O | GLY | C | 103 | 69.283 | 13.954 | 10.483 | 1.00 | 109.62 | | O |
| ANISOU | 786 | O | GLY | C | 103 | 14811 | 15444 | 11395 | −4101 | −942 | 668 | O |
| ATOM | 787 | N | GLU | C | 104 | 70.437 | 15.339 | 9.080 | 1.00 | 107.08 | | N |
| ANISOU | 787 | N | GLU | C | 104 | 14315 | 14771 | 11601 | −3845 | −933 | 587 | N |
| ATOM | 788 | CA | GLU | C | 104 | 71.611 | 14.484 | 8.867 | 1.00 | 107.95 | | C |
| ANISOU | 788 | CA | GLU | C | 104 | 14484 | 14712 | 11820 | −3796 | −1009 | 808 | C |
| ATOM | 789 | C | GLU | C | 104 | 72.260 | 14.247 | 10.233 | 1.00 | 116.67 | | C |
| ANISOU | 789 | C | GLU | C | 104 | 15606 | 15949 | 12775 | −3852 | −1179 | 831 | C |
| ATOM | 790 | O | GLU | C | 104 | 72.503 | 13.097 | 10.593 | 1.00 | 117.15 | | O |
| ANISOU | 790 | O | GLU | C | 104 | 15807 | 16007 | 12699 | −3892 | −1245 | 1015 | O |
| ATOM | 791 | CB | GLU | C | 104 | 72.606 | 15.147 | 7.884 | 1.00 | 108.31 | | C |
| ANISOU | 791 | CB | GLU | C | 104 | 14412 | 14537 | 12203 | −3656 | −1000 | 818 | C |
| ATOM | 792 | CG | GLU | C | 104 | 73.625 | 14.204 | 7.248 | 1.00 | 116.43 | | C |
| ANISOU | 792 | CG | GLU | C | 104 | 15478 | 15400 | 13359 | −3568 | −1029 | 1026 | C |
| ATOM | 793 | CD | GLU | C | 104 | 74.823 | 13.756 | 8.074 | 1.00 | 136.84 | | C |
| ANISOU | 793 | CD | GLU | C | 104 | 18058 | 18003 | 15930 | −3543 | −1206 | 1110 | C |
| ATOM | 794 | OE1 | GLU | C | 104 | 75.265 | 14.501 | 8.981 | 1.00 | 125.50 | | O |
| ANISOU | 794 | OE1 | GLU | C | 104 | 16527 | 16670 | 14490 | −3572 | −1314 | 995 | O |
| ATOM | 795 | OE2 | GLU | C | 104 | 75.343 | 12.655 | 7.780 | 1.00 | 129.82 | | O |
| ANISOU | 795 | OE2 | GLU | C | 104 | 17261 | 17022 | 15042 | −3476 | −1249 | 1279 | O |
| ATOM | 796 | N | THR | C | 105 | 72.451 | 15.335 | 11.014 | 1.00 | 116.20 | | N |
| ANISOU | 796 | N | THR | C | 105 | 15419 | 16009 | 12725 | −3853 | −1258 | 635 | N |
| ATOM | 797 | CA | THR | C | 105 | 73.003 | 15.348 | 12.371 | 1.00 | 118.77 | | C |
| ANISOU | 797 | CA | THR | C | 105 | 15727 | 16502 | 12897 | −3898 | −1425 | 600 | C |
| ATOM | 798 | C | THR | C | 105 | 72.175 | 14.455 | 13.327 | 1.00 | 126.42 | | C |
| ANISOU | 798 | C | THR | C | 105 | 16842 | 17708 | 13484 | −4047 | −1434 | 674 | C |
| ATOM | 799 | O | THR | C | 105 | 72.768 | 13.705 | 14.103 | 1.00 | 127.62 | | O |
| ANISOU | 799 | O | THR | C | 105 | 17085 | 17905 | 13498 | −4075 | −1565 | 820 | O |
| ATOM | 800 | CB | THR | C | 105 | 73.152 | 16.810 | 12.836 | 1.00 | 126.27 | | C |
| ANISOU | 800 | CB | THR | C | 105 | 16508 | 17523 | 13944 | −3870 | −1484 | 328 | C |
| ATOM | 801 | CG2 | THR | C | 105 | 73.574 | 16.937 | 14.293 | 1.00 | 128.64 | | C |
| ANISOU | 801 | CG2 | THR | C | 105 | 16780 | 18070 | 14027 | −3927 | −1641 | 228 | C |
| ATOM | 802 | OG1 | THR | C | 105 | 74.108 | 17.467 | 12.000 | 1.00 | 123.08 | | O |
| ANISOU | 802 | OG1 | THR | C | 105 | 15995 | 16889 | 13880 | −3772 | −1513 | 330 | O |
| ATOM | 803 | N | LYS | C | 106 | 70.818 | 14.519 | 13.238 | 1.00 | 124.43 | | N |
| ANISOU | 803 | N | LYS | C | 106 | 16609 | 17612 | 13056 | −4146 | −1299 | 580 | N |
| ATOM | 804 | CA | LYS | C | 106 | 69.862 | 13.747 | 14.054 | 1.00 | 126.37 | | C |
| ANISOU | 804 | CA | LYS | C | 106 | 16971 | 18122 | 12922 | −4333 | −1272 | 637 | C |
| ATOM | 805 | C | LYS | C | 106 | 69.794 | 12.263 | 13.675 | 1.00 | 132.72 | | C |
| ANISOU | 805 | C | LYS | C | 106 | 17991 | 18792 | 13646 | −4417 | −1245 | 933 | C |
| ATOM | 806 | O | LYS | C | 106 | 69.643 | 11.414 | 14.558 | 1.00 | 133.60 | | O |
| ANISOU | 806 | O | LYS | C | 106 | 18249 | 19036 | 13477 | −4564 | −1312 | 1082 | O |
| ATOM | 807 | CB | LYS | C | 106 | 68.454 | 14.380 | 14.019 | 1.00 | 128.23 | | C |
| ANISOU | 807 | CB | LYS | C | 106 | 17114 | 18599 | 13010 | −4401 | −1133 | 402 | C |
| ATOM | 808 | CG | LYS | C | 106 | 68.181 | 15.373 | 15.155 | 1.00 | 137.62 | | C |
| ANISOU | 808 | CG | LYS | C | 106 | 18157 | 20090 | 14041 | −4408 | −1203 | 124 | C |
| ATOM | 809 | CD | LYS | C | 106 | 67.497 | 14.724 | 16.371 | 1.00 | 142.66 | | C |
| ANISOU | 809 | CD | LYS | C | 106 | 18849 | 21121 | 14235 | −4616 | −1203 | 140 | C |
| ATOM | 810 | CE | LYS | C | 106 | 67.548 | 15.585 | 17.618 | 1.00 | 141.03 | | C |
| ANISOU | 810 | CE | LYS | C | 106 | 18506 | 21223 | 13857 | −4605 | −1309 | −113 | C |
| ATOM | 811 | NZ | LYS | C | 106 | 68.870 | 15.514 | 18.296 | 1.00 | 139.13 | | N |
| ANISOU | 811 | NZ | LYS | C | 106 | 18300 | 20911 | 13652 | −4561 | −1498 | −8 | N |
| ATOM | 812 | N | VAL | C | 107 | 69.889 | 11.957 | 12.366 | 1.00 | 129.97 | | N |
| ANISOU | 812 | N | VAL | C | 107 | 17673 | 18176 | 13535 | −4326 | −1155 | 1017 | N |
| ATOM | 813 | CA | VAL | C | 107 | 69.868 | 10.586 | 11.853 | 1.00 | 130.74 | | C |
| ANISOU | 813 | CA | VAL | C | 107 | 17973 | 18093 | 13607 | −4374 | −1139 | 1269 | C |
| ATOM | 814 | C | VAL | C | 107 | 71.215 | 9.901 | 12.128 | 1.00 | 138.31 | | C |
| ANISOU | 814 | C | VAL | C | 107 | 19036 | 18869 | 14645 | −4273 | −1322 | 1465 | C |
| ATOM | 815 | O | VAL | C | 107 | 71.225 | 8.818 | 12.715 | 1.00 | 140.24 | | O |
| ANISOU | 815 | O | VAL | C | 107 | 19490 | 19106 | 14691 | −4377 | −1410 | 1664 | O |
| ATOM | 816 | CB | VAL | C | 107 | 69.426 | 10.517 | 10.371 | 1.00 | 132.39 | | C |
| ANISOU | 816 | CB | VAL | C | 107 | 18163 | 18115 | 14025 | −4295 | −986 | 1257 | C |
| ATOM | 817 | CG1 | VAL | C | 107 | 69.776 | 9.173 | 9.748 | 1.00 | 132.09 | | C |
| ANISOU | 817 | CG1 | VAL | C | 107 | 18318 | 17824 | 14046 | −4274 | −1014 | 1498 | C |
| ATOM | 818 | CG2 | VAL | C | 107 | 67.934 | 10.795 | 10.239 | 1.00 | 131.82 | | C |
| ANISOU | 818 | CG2 | VAL | C | 107 | 18044 | 18255 | 13787 | −4428 | −823 | 1107 | C |
| ATOM | 819 | N | SER | C | 108 | 72.342 | 10.548 | 11.739 | 1.00 | 135.39 | | N |
| ANISOU | 819 | N | SER | C | 108 | 18521 | 18364 | 14556 | −4077 | −1388 | 1406 | N |
| ATOM | 820 | CA | SER | C | 108 | 73.703 | 10.049 | 11.986 | 1.00 | 136.97 | | C |
| ANISOU | 820 | CA | SER | C | 108 | 18762 | 18434 | 14847 | −3947 | −1571 | 1543 | C |
| ATOM | 821 | C | SER | C | 108 | 74.021 | 10.131 | 13.487 | 1.00 | 144.78 | | C |
| ANISOU | 821 | C | SER | C | 108 | 19768 | 19638 | 15604 | −4017 | −1734 | 1532 | C |
| ATOM | 822 | O | SER | C | 108 | 73.299 | 10.807 | 14.223 | 1.00 | 144.71 | | O |
| ANISOU | 822 | O | SER | C | 108 | 19694 | 19877 | 15410 | −4145 | −1691 | 1383 | O |
| ATOM | 823 | CB | SER | C | 108 | 74.732 | 10.839 | 11.180 | 1.00 | 139.35 | | C |
| ANISOU | 823 | CB | SER | C | 108 | 18860 | 18593 | 15493 | −3754 | −1573 | 1455 | C |
| ATOM | 824 | OG | SER | C | 108 | 74.588 | 10.639 | 9.782 | 1.00 | 145.43 | | O |
| ANISOU | 824 | OG | SER | C | 108 | 19628 | 19169 | 16459 | −3671 | −1442 | 1498 | O |
| ATOM | 825 | N | LYS | C | 109 | 75.072 | 9.409 | 13.945 | 1.00 | 144.55 | | N |
| ANISOU | 825 | N | LYS | C | 109 | 19828 | 19531 | 15563 | −3921 | −1928 | 1683 | N |
| ATOM | 826 | CA | LYS | C | 109 | 75.508 | 9.319 | 15.353 | 1.00 | 147.58 | | C |
| ANISOU | 826 | CA | LYS | C | 109 | 20254 | 20104 | 15717 | −3959 | −2114 | 1708 | C |
| ATOM | 827 | C | LYS | C | 109 | 74.516 | 8.504 | 16.219 | 1.00 | 155.34 | | C |
| ANISOU | 827 | C | LYS | C | 109 | 21473 | 21244 | 16306 | −4186 | −2118 | 1853 | C |
| ATOM | 828 | O | LYS | C | 109 | 74.899 | 8.007 | 17.284 | 1.00 | 157.32 | | O |
| ANISOU | 828 | O | LYS | C | 109 | 21853 | 21586 | 16335 | −4213 | −2294 | 1977 | O |
| ATOM | 829 | CB | LYS | C | 109 | 75.821 | 10.710 | 15.962 | 1.00 | 149.99 | | C |
| ANISOU | 829 | CB | LYS | C | 109 | 20303 | 20613 | 16074 | −3939 | −2141 | 1447 | C |
| ATOM | 830 | CG | LYS | C | 109 | 76.718 | 10.671 | 17.192 | 1.00 | 165.28 | | C |
| ANISOU | 830 | CG | LYS | C | 109 | 22231 | 22704 | 17865 | −3898 | −2367 | 1455 | C |
| ATOM | 831 | CD | LYS | C | 109 | 77.925 | 11.587 | 17.050 | 1.00 | 174.41 | | C |
| ANISOU | 831 | CD | LYS | C | 109 | 23135 | 23829 | 19304 | −3737 | −2458 | 1288 | C |
| ATOM | 832 | CE | LYS | C | 109 | 79.218 | 10.931 | 17.485 | 1.00 | 184.63 | | C |
| ANISOU | 832 | CE | LYS | C | 109 | 24460 | 25070 | 20620 | −3578 | −2683 | 1415 | C |
| ATOM | 833 | NZ | LYS | C | 109 | 79.265 | 10.648 | 18.947 | 1.00 | 192.97 | | N |
| ANISOU | 833 | NZ | LYS | C | 109 | 25627 | 26352 | 21341 | −3629 | −2867 | 1468 | N |
| ATOM | 834 | N | LEU | C | 110 | 73.260 | 8.341 | 15.738 | 1.00 | 152.59 | | N |
| ANISOU | 834 | N | LEU | C | 110 | 21181 | 20933 | 15864 | −4354 | −1928 | 1844 | N |
| ATOM | 835 | CA | LEU | C | 110 | 72.188 | 7.595 | 16.405 | 1.00 | 154.89 | | C |
| ANISOU | 835 | CA | LEU | C | 110 | 21674 | 21393 | 15784 | −4617 | −1893 | 1976 | C |
| ATOM | 836 | C | LEU | C | 110 | 72.257 | 6.085 | 16.124 | 1.00 | 161.04 | | C |
| ANISOU | 836 | C | LEU | C | 110 | 22769 | 21910 | 16509 | −4661 | −1970 | 2285 | C |
| ATOM | 837 | O | LEU | C | 110 | 71.757 | 5.294 | 16.932 | 1.00 | 163.41 | | O |
| ANISOU | 837 | O | LEU | C | 110 | 23293 | 22309 | 16487 | −4868 | −2031 | 2470 | O |
| ATOM | 838 | CB | LEU | C | 110 | 70.800 | 8.176 | 16.051 | 1.00 | 153.79 | | C |
| ANISOU | 838 | CB | LEU | C | 110 | 21422 | 21447 | 15563 | −4780 | −1661 | 1800 | C |
| ATOM | 839 | CG | LEU | C | 110 | 69.663 | 7.960 | 17.074 | 1.00 | 160.79 | | C |
| ANISOU | 839 | CG | LEU | C | 110 | 22379 | 22695 | 16019 | −5072 | −1608 | 1812 | C |
| ATOM | 840 | CD1 | LEU | C | 110 | 69.959 | 8.644 | 18.418 | 1.00 | 162.77 | | C |
| ANISOU | 840 | CD1 | LEU | C | 110 | 22500 | 23272 | 16071 | −5076 | −1711 | 1666 | C |
| ATOM | 841 | CD2 | LEU | C | 110 | 68.347 | 8.469 | 16.535 | 1.00 | 161.97 | | C |
| ANISOU | 841 | CD2 | LEU | C | 110 | 22411 | 23008 | 16121 | −5201 | −1380 | 1638 | C |
| ATOM | 842 | N | TYR | C | 111 | 72.907 | 5.684 | 15.011 | 1.00 | 156.44 | | N |
| ANISOU | 842 | N | TYR | C | 111 | 22213 | 20994 | 16233 | −4465 | −1982 | 2342 | N |
| ATOM | 843 | CA | TYR | C | 111 | 73.081 | 4.274 | 14.656 | 1.00 | 157.62 | | C |
| ANISOU | 843 | CA | TYR | C | 111 | 22662 | 20847 | 16378 | −4455 | −2082 | 2603 | C |
| ATOM | 844 | C | TYR | C | 111 | 74.269 | 3.626 | 15.375 | 1.00 | 164.31 | | C |
| ANISOU | 844 | C | TYR | C | 111 | 23668 | 21586 | 17177 | −4307 | −2361 | 2774 | C |
| ATOM | 845 | O | TYR | C | 111 | 74.471 | 2.413 | 15.265 | 1.00 | 165.44 | | O |
| ANISOU | 845 | O | TYR | C | 111 | 24102 | 21477 | 17282 | −4291 | −2494 | 3002 | O |
| ATOM | 846 | CB | TYR | C | 111 | 73.137 | 4.082 | 13.137 | 1.00 | 156.71 | | C |
| ANISOU | 846 | CB | TYR | C | 111 | 22506 | 20455 | 16581 | −4298 | −1982 | 2569 | C |
| ATOM | 847 | CG | TYR | C | 111 | 71.767 | 3.811 | 12.560 | 1.00 | 157.79 | | C |
| ANISOU | 847 | CG | TYR | C | 111 | 22716 | 20599 | 16638 | −4515 | −1783 | 2568 | C |
| ATOM | 848 | CD1 | TYR | C | 111 | 71.331 | 2.508 | 12.327 | 1.00 | 161.09 | | C |
| ANISOU | 848 | CD1 | TYR | C | 111 | 23438 | 20806 | 16963 | −4639 | −1824 | 2781 | C |
| ATOM | 849 | CD2 | TYR | C | 111 | 70.871 | 4.850 | 12.325 | 1.00 | 156.66 | | C |
| ANISOU | 849 | CD2 | TYR | C | 111 | 22345 | 20684 | 16494 | −4609 | −1570 | 2349 | C |
| ATOM | 850 | CE1 | TYR | C | 111 | 70.051 | 2.250 | 11.839 | 1.00 | 160.95 | | C |
| ANISOU | 850 | CE1 | TYR | C | 111 | 23474 | 20821 | 16859 | −4866 | −1644 | 2769 | C |
| ATOM | 851 | CE2 | TYR | C | 111 | 69.585 | 4.603 | 11.853 | 1.00 | 156.86 | | C |
| ANISOU | 851 | CE2 | TYR | C | 111 | 22418 | 20761 | 16422 | −4809 | −1395 | 2330 | C |
| ATOM | 852 | CZ | TYR | C | 111 | 69.184 | 3.302 | 11.598 | 1.00 | 164.58 | | C |
| ANISOU | 852 | CZ | TYR | C | 111 | 23677 | 21543 | 17313 | −4947 | −1426 | 2539 | C |
| ATOM | 853 | OH | TYR | C | 111 | 67.925 | 3.064 | 11.111 | 1.00 | 163.97 | | O |
| ANISOU | 853 | OH | TYR | C | 111 | 23625 | 21531 | 17146 | −5157 | −1253 | 2505 | O |
| ATOM | 854 | N | LYS | C | 112 | 75.024 | 4.436 | 16.147 | 1.00 | 161.83 | | N |
| ANISOU | 854 | N | LYS | C | 112 | 23170 | 21464 | 16856 | −4200 | −2464 | 2656 | N |
| ATOM | 855 | CA | LYS | C | 112 | 76.144 | 4.011 | 16.991 | 1.00 | 164.27 | | C |
| ANISOU | 855 | CA | LYS | C | 112 | 23580 | 21747 | 17088 | −4052 | −2738 | 2777 | C |
| ATOM | 856 | C | LYS | C | 112 | 75.563 | 3.255 | 18.200 | 1.00 | 171.85 | | C |
| ANISOU | 856 | C | LYS | C | 112 | 24834 | 22835 | 17627 | −4299 | −2833 | 2996 | C |
| ATOM | 857 | O | LYS | C | 112 | 76.125 | 2.244 | 18.633 | 1.00 | 174.11 | | O |
| ANISOU | 857 | O | LYS | C | 112 | 25398 | 22950 | 17805 | −4237 | −3056 | 3233 | O |
| ATOM | 858 | CB | LYS | C | 112 | 76.936 | 5.238 | 17.486 | 1.00 | 166.05 | | C |
| ANISOU | 858 | CB | LYS | C | 112 | 23493 | 22196 | 17403 | −3918 | −2792 | 2549 | C |
| ATOM | 859 | CG | LYS | C | 112 | 77.774 | 5.939 | 16.419 | 1.00 | 172.02 | | C |
| ANISOU | 859 | CG | LYS | C | 112 | 23968 | 22828 | 18562 | −3679 | −2741 | 2366 | C |
| ATOM | 860 | CD | LYS | C | 112 | 78.318 | 7.280 | 16.915 | 1.00 | 177.87 | | C |
| ANISOU | 860 | CD | LYS | C | 112 | 24397 | 23805 | 19381 | −3631 | −2756 | 2118 | C |
| ATOM | 861 | CE | LYS | C | 112 | 79.546 | 7.183 | 17.793 | 1.00 | 185.76 | | C |
| ANISOU | 861 | CE | LYS | C | 112 | 25360 | 24873 | 20348 | −3463 | −3019 | 2134 | C |
| ATOM | 862 | NZ | LYS | C | 112 | 80.773 | 6.913 | 17.002 | 1.00 | 191.88 | | N |
| ANISOU | 862 | NZ | LYS | C | 112 | 26032 | 25448 | 21427 | −3193 | −3110 | 2135 | N |
| ATOM | 863 | N | LEU | C | 113 | 74.427 | 3.761 | 18.730 | 1.00 | 168.45 | | N |
| ANISOU | 863 | N | LEU | C | 113 | 24338 | 22716 | 16950 | −4577 | −2666 | 2912 | N |
| ATOM | 864 | CA | LEU | C | 113 | 73.688 | 3.210 | 19.869 | 1.00 | 171.10 | | C |
| ANISOU | 864 | CA | LEU | C | 113 | 24898 | 23266 | 16848 | −4872 | −2700 | 3092 | C |
| ATOM | 865 | C | LEU | C | 113 | 72.796 | 2.041 | 19.423 | 1.00 | 175.26 | | C |
| ANISOU | 865 | C | LEU | C | 113 | 25736 | 23590 | 17264 | −5103 | −2634 | 3334 | C |
| ATOM | 866 | O | LEU | C | 113 | 72.593 | 1.095 | 20.191 | 1.00 | 177.68 | | O |
| ANISOU | 866 | O | LEU | C | 113 | 26360 | 23879 | 17271 | −5285 | −2762 | 3609 | O |
| ATOM | 867 | CB | LEU | C | 113 | 72.838 | 4.315 | 20.532 | 1.00 | 170.85 | | C |
| ANISOU | 867 | CB | LEU | C | 113 | 24618 | 23687 | 16610 | −5058 | −2533 | 2855 | C |
| ATOM | 868 | CG | LEU | C | 113 | 73.607 | 5.482 | 21.172 | 1.00 | 175.32 | | C |
| ANISOU | 868 | CG | LEU | C | 113 | 24902 | 24482 | 17230 | −4878 | −2618 | 2610 | C |
| ATOM | 869 | CD1 | LEU | C | 113 | 72.984 | 6.818 | 20.805 | 1.00 | 172.97 | | C |
| ANISOU | 869 | CD1 | LEU | C | 113 | 24272 | 24387 | 17062 | −4888 | −2407 | 2264 | C |
| ATOM | 870 | CD2 | LEU | C | 113 | 73.710 | 5.319 | 22.684 | 1.00 | 180.82 | | C |
| ANISOU | 870 | CD2 | LEU | C | 113 | 25700 | 25484 | 17521 | −4992 | −2776 | 2706 | C |
| ATOM | 871 | N | ALA | C | 114 | 72.277 | 2.113 | 18.175 | 1.00 | 168.99 | | N |
| ANISOU | 871 | N | ALA | C | 114 | 24859 | 22639 | 16709 | −5102 | −2442 | 3234 | N |
| ATOM | 872 | CA | ALA | C | 114 | 71.416 | 1.102 | 17.557 | 1.00 | 196.16 | | C |
| ANISOU | 872 | CA | ALA | C | 114 | 28553 | 25876 | 20102 | −5312 | −2361 | 3411 | C |
| ATOM | 873 | C | ALA | C | 114 | 72.240 | 0.115 | 16.731 | 1.00 | 220.10 | | C |
| ANISOU | 873 | C | ALA | C | 114 | 31805 | 28429 | 23394 | −5078 | −2525 | 3568 | C |
| ATOM | 874 | O | ALA | C | 114 | 73.133 | −0.545 | 17.259 | 1.00 | 181.53 | | O |
| ANISOU | 874 | O | ALA | C | 114 | 27136 | 23376 | 18463 | −4945 | −2782 | 3755 | O |
| ATOM | 875 | CB | ALA | C | 114 | 70.374 | 1.774 | 16.676 | 1.00 | 194.09 | | C |
| ANISOU | 875 | CB | ALA | C | 114 | 28061 | 25741 | 19941 | −5420 | −2073 | 3186 | C |
| ATOM | 876 | N | LEU | C | 126 | 64.683 | −1.006 | 15.097 | 1.00 | 154.16 | | N |
| ANISOU | 876 | N | LEU | C | 126 | 23538 | 20785 | 14249 | −6891 | −1398 | 3491 | N |
| ATOM | 877 | CA | LEU | C | 126 | 65.914 | −0.216 | 15.194 | 1.00 | 152.49 | | C |
| ANISOU | 877 | CA | LEU | C | 126 | 23172 | 20507 | 14260 | −6482 | −1518 | 3381 | C |
| ATOM | 878 | C | LEU | C | 126 | 66.193 | 0.654 | 13.934 | 1.00 | 152.85 | | C |
| ANISOU | 878 | C | LEU | C | 126 | 22923 | 20455 | 14697 | −6148 | −1403 | 3100 | C |
| ATOM | 879 | O | LEU | C | 126 | 67.057 | 1.537 | 13.979 | 1.00 | 150.33 | | O |
| ANISOU | 879 | O | LEU | C | 126 | 22394 | 20179 | 14546 | −5862 | −1445 | 2949 | O |
| ATOM | 880 | CB | LEU | C | 126 | 67.143 | −1.083 | 15.574 | 1.00 | 154.40 | | C |
| ANISOU | 880 | CB | LEU | C | 126 | 23732 | 20372 | 14563 | −6312 | −1813 | 3654 | C |
| ATOM | 881 | CG | LEU | C | 126 | 67.209 | −2.539 | 15.087 | 1.00 | 160.59 | | C |
| ANISOU | 881 | CG | LEU | C | 126 | 24909 | 20692 | 15416 | −6379 | −1944 | 3917 | C |
| ATOM | 882 | CD1 | LEU | C | 126 | 67.828 | −2.635 | 13.694 | 1.00 | 157.97 | | C |
| ANISOU | 882 | CD1 | LEU | C | 126 | 24515 | 19993 | 15513 | −6016 | −1964 | 3800 | C |
| ATOM | 883 | CD2 | LEU | C | 126 | 68.000 | −3.393 | 16.059 | 1.00 | 166.26 | | C |
| ANISOU | 883 | CD2 | LEU | C | 126 | 25992 | 21213 | 15968 | −6388 | −2233 | 4232 | C |
| ATOM | 884 | N | ARG | C | 127 | 65.430 | 0.429 | 12.840 | 1.00 | 148.88 | | N |
| ANISOU | 884 | N | ARG | C | 127 | 22404 | 19841 | 14325 | −6206 | −1257 | 3031 | N |
| ATOM | 885 | CA | ARG | C | 127 | 65.514 | 1.184 | 11.579 | 1.00 | 145.51 | | C |
| ANISOU | 885 | CA | ARG | C | 127 | 21720 | 19338 | 14231 | −5928 | −1131 | 2787 | C |
| ATOM | 886 | C | ARG | C | 127 | 64.298 | 2.113 | 11.389 | 1.00 | 148.87 | | C |
| ANISOU | 886 | C | ARG | C | 127 | 21854 | 20150 | 14560 | −6055 | −890 | 2519 | C |
| ATOM | 887 | O | ARG | C | 127 | 64.361 | 3.055 | 10.603 | 1.00 | 145.15 | | O |
| ANISOU | 887 | O | ARG | C | 127 | 21127 | 19709 | 14314 | −5814 | −795 | 2289 | O |
| ATOM | 888 | CB | ARG | C | 127 | 65.664 | 0.231 | 10.378 | 1.00 | 145.00 | | C |
| ANISOU | 888 | CB | ARG | C | 127 | 21833 | 18860 | 14400 | −5835 | −1162 | 2877 | C |
| ATOM | 889 | CG | ARG | C | 127 | 66.219 | 0.896 | 9.118 | 1.00 | 151.05 | | C |
| ANISOU | 889 | CG | ARG | C | 127 | 22376 | 19484 | 15532 | −5472 | −1096 | 2682 | C |
| ATOM | 890 | CD | ARG | C | 127 | 65.195 | 1.041 | 8.003 | 1.00 | 158.38 | | C |
| ANISOU | 890 | CD | ARG | C | 127 | 23179 | 20467 | 16531 | −5532 | −890 | 2519 | C |
| ATOM | 891 | NE | ARG | C | 127 | 65.331 | 0.007 | 6.971 | 1.00 | 165.59 | | N |
| ANISOU | 891 | NE | ARG | C | 127 | 24277 | 21016 | 17624 | −5453 | −938 | 2606 | N |
| ATOM | 892 | CZ | ARG | C | 127 | 65.791 | 0.217 | 5.739 | 1.00 | 174.81 | | C |
| ANISOU | 892 | CZ | ARG | C | 127 | 25329 | 22003 | 19087 | −5148 | −909 | 2498 | C |
| ATOM | 893 | NH1 | ARG | C | 127 | 66.168 | 1.432 | 5.361 | 1.00 | 156.85 | | N |
| ANISOU | 893 | NH1 | ARG | C | 127 | 22766 | 19858 | 16971 | −4911 | −828 | 2320 | N |
| ATOM | 894 | NH2 | ARG | C | 127 | 65.874 | −0.786 | 4.875 | 1.00 | 161.22 | | N |
| ANISOU | 894 | NH2 | ARG | C | 127 | 23786 | 19973 | 17499 | −5084 | −964 | 2565 | N |
| ATOM | 895 | N | GLN | C | 128 | 63.201 | 1.831 | 12.118 | 1.00 | 149.07 | | N |
| ANISOU | 895 | N | GLN | C | 128 | 21922 | 20477 | 14241 | −6433 | −798 | 2552 | N |
| ATOM | 896 | CA | GLN | C | 128 | 61.914 | 2.546 | 12.111 | 1.00 | 149.06 | | C |
| ANISOU | 896 | CA | GLN | C | 128 | 21659 | 20905 | 14073 | −6601 | −580 | 2302 | C |
| ATOM | 897 | C | GLN | C | 128 | 62.072 | 4.072 | 12.284 | 1.00 | 151.76 | | C |
| ANISOU | 897 | C | GLN | C | 128 | 21666 | 21509 | 14489 | −6357 | −525 | 2003 | C |
| ATOM | 898 | O | GLN | C | 128 | 61.436 | 4.837 | 11.555 | 1.00 | 149.08 | | O |
| ANISOU | 898 | O | GLN | C | 128 | 21093 | 21291 | 14259 | −6252 | −379 | 1750 | O |
| ATOM | 899 | CB | GLN | C | 128 | 60.964 | 1.981 | 13.204 | 1.00 | 154.00 | | C |
| ANISOU | 899 | CB | GLN | C | 128 | 22384 | 21876 | 14253 | −7058 | −533 | 2418 | C |
| ATOM | 900 | CG | GLN | C | 128 | 60.914 | 0.444 | 13.336 | 1.00 | 173.09 | | C |
| ANISOU | 900 | CG | GLN | C | 128 | 25186 | 24022 | 16557 | −7358 | −620 | 2757 | C |
| ATOM | 901 | CD | GLN | C | 128 | 59.879 | −0.232 | 12.463 | 1.00 | 193.46 | | C |
| ANISOU | 901 | CD | GLN | C | 128 | 27788 | 26557 | 19159 | −7567 | −479 | 2723 | C |
| ATOM | 902 | NE2 | GLN | C | 128 | 59.058 | −1.077 | 13.071 | 1.00 | 187.56 | | N |
| ANISOU | 902 | NE2 | GLN | C | 128 | 27241 | 25911 | 18113 | −8019 | −457 | 2915 | N |
| ATOM | 903 | OE1 | GLN | C | 128 | 59.835 | −0.057 | 11.238 | 1.00 | 187.26 | | O |
| ANISOU | 903 | OE1 | GLN | C | 128 | 26861 | 25632 | 18656 | −7338 | −400 | 2541 | O |
| ATOM | 904 | N | MET | C | 129 | 62.931 | 4.497 | 13.243 | 1.00 | 150.10 | | N |
| ANISOU | 904 | N | MET | C | 129 | 21447 | 21365 | 14221 | −6261 | −659 | 2032 | N |
| ATOM | 905 | CA | MET | C | 129 | 63.234 | 5.891 | 13.602 | 1.00 | 149.13 | | C |
| ANISOU | 905 | CA | MET | C | 129 | 21048 | 21456 | 14159 | −6045 | −656 | 1771 | C |
| ATOM | 906 | C | MET | C | 129 | 63.638 | 6.732 | 12.383 | 1.00 | 148.88 | | C |
| ANISOU | 906 | C | MET | C | 129 | 20840 | 21204 | 14525 | −5707 | −610 | 1584 | C |
| ATOM | 907 | O | MET | C | 129 | 63.222 | 7.885 | 12.272 | 1.00 | 146.87 | | O |
| ANISOU | 907 | O | MET | C | 129 | 20335 | 21161 | 14307 | −5600 | −521 | 1304 | O |
| ATOM | 908 | CB | MET | C | 129 | 64.286 | 5.928 | 14.748 | 1.00 | 153.39 | | C |
| ANISOU | 908 | CB | MET | C | 129 | 21671 | 21998 | 14614 | −5988 | −851 | 1893 | C |
| ATOM | 909 | CG | MET | C | 129 | 65.428 | 6.930 | 14.576 | 1.00 | 155.87 | | C |
| ANISOU | 909 | CG | MET | C | 129 | 21821 | 22172 | 15229 | −5635 | −945 | 1748 | C |
| ATOM | 910 | SD | MET | C | 129 | 66.787 | 6.297 | 13.546 | 1.00 | 159.13 | | S |
| ANISOU | 910 | SD | MET | C | 129 | 22384 | 22031 | 16047 | −5355 | −1076 | 1940 | S |
| ATOM | 911 | CE | MET | C | 129 | 67.456 | 7.830 | 12.905 | 1.00 | 153.15 | | C |
| ANISOU | 911 | CE | MET | C | 129 | 21315 | 21234 | 15639 | −5026 | −1050 | 1656 | C |
| ATOM | 912 | N | PHE | C | 130 | 64.426 | 6.122 | 11.470 | 1.00 | 144.25 | | N |
| ANISOU | 912 | N | PHE | C | 130 | 20394 | 20196 | 14219 | −5543 | −677 | 1743 | N |
| ATOM | 913 | CA | PHE | C | 130 | 64.960 | 6.691 | 10.231 | 1.00 | 141.33 | | C |
| ANISOU | 913 | CA | PHE | C | 130 | 19900 | 19578 | 14221 | −5239 | −644 | 1634 | C |
| ATOM | 914 | C | PHE | C | 130 | 63.873 | 7.282 | 9.335 | 1.00 | 142.36 | | C |
| ANISOU | 914 | C | PHE | C | 130 | 19853 | 19843 | 14394 | −5231 | −454 | 1411 | C |
| ATOM | 915 | O | PHE | C | 130 | 63.983 | 8.439 | 8.934 | 1.00 | 140.08 | | O |
| ANISOU | 915 | O | PHE | C | 130 | 19358 | 19591 | 14273 | −5029 | −410 | 1204 | O |
| ATOM | 916 | CB | PHE | C | 130 | 65.725 | 5.603 | 9.449 | 1.00 | 143.09 | | C |
| ANISOU | 916 | CB | PHE | C | 130 | 20335 | 19390 | 14645 | −5137 | −732 | 1858 | C |
| ATOM | 917 | CG | PHE | C | 130 | 66.982 | 6.027 | 8.731 | 1.00 | 143.31 | | C |
| ANISOU | 917 | CG | PHE | C | 130 | 20278 | 19156 | 15018 | −4806 | −800 | 1839 | C |
| ATOM | 918 | CD1 | PHE | C | 130 | 66.949 | 7.017 | 7.759 | 1.00 | 144.55 | | C |
| ANISOU | 918 | CD1 | PHE | C | 130 | 20219 | 19304 | 15400 | −4621 | −683 | 1648 | C |
| ATOM | 919 | CD2 | PHE | C | 130 | 68.187 | 5.382 | 8.969 | 1.00 | 146.70 | | C |
| ANISOU | 919 | CD2 | PHE | C | 130 | 20848 | 19350 | 15540 | −4681 | −984 | 2020 | C |
| ATOM | 920 | CE1 | PHE | C | 130 | 68.113 | 7.395 | 7.086 | 1.00 | 144.32 | | C |
| ANISOU | 920 | CE1 | PHE | C | 130 | 20105 | 19058 | 15670 | −4352 | −734 | 1646 | C |
| ATOM | 921 | CE2 | PHE | C | 130 | 69.352 | 5.763 | 8.300 | 1.00 | 148.24 | | C |
| ANISOU | 921 | CE2 | PHE | C | 130 | 20934 | 19353 | 16039 | −4390 | −1037 | 1990 | C |
| ATOM | 922 | CZ | PHE | C | 130 | 69.304 | 6.754 | 7.349 | 1.00 | 144.17 | | C |
| ANISOU | 922 | CZ | PHE | C | 130 | 20198 | 18847 | 15735 | −4245 | −903 | 1812 | C |
| ATOM | 923 | N | ILE | C | 131 | 62.830 | 6.485 | 9.030 | 1.00 | 138.99 | | N |
| ANISOU | 923 | N | ILE | C | 131 | 19514 | 19486 | 13809 | −5457 | −354 | 1456 | N |
| ATOM | 924 | CA | ILE | C | 131 | 61.720 | 6.853 | 8.147 | 1.00 | 137.15 | | C |
| ANISOU | 924 | CA | ILE | C | 131 | 19129 | 19395 | 13586 | −5469 | −181 | 1256 | C |
| ATOM | 925 | C | ILE | C | 131 | 60.871 | 8.006 | 8.699 | 1.00 | 139.67 | | C |
| ANISOU | 925 | C | ILE | C | 131 | 19193 | 20129 | 13745 | −5480 | −92 | 965 | C |
| ATOM | 926 | O | ILE | C | 131 | 60.350 | 8.799 | 7.902 | 1.00 | 137.84 | | O |
| ANISOU | 926 | O | ILE | C | 131 | 18785 | 19949 | 13638 | −5317 | 3 | 747 | O |
| ATOM | 927 | CB | ILE | C | 131 | 60.822 | 5.663 | 7.700 | 1.00 | 141.36 | | C |
| ANISOU | 927 | CB | ILE | C | 131 | 19813 | 19925 | 13973 | −5741 | −106 | 1366 | C |
| ATOM | 928 | CG1 | ILE | C | 131 | 61.243 | 4.276 | 8.326 | 1.00 | 143.28 | | C |
| ANISOU | 928 | CG1 | ILE | C | 131 | 20386 | 19896 | 14157 | −5908 | −247 | 1697 | C |
| ATOM | 929 | CG2 | ILE | C | 131 | 60.751 | 5.620 | 6.172 | 1.00 | 140.08 | | C |
| ANISOU | 929 | CG2 | ILE | C | 131 | 19575 | 19618 | 14032 | −5581 | −2 | 1256 | C |
| ATOM | 930 | CD1 | ILE | C | 131 | 62.401 | 3.442 | 7.655 | 1.00 | 146.76 | | C |
| ANISOU | 930 | CD1 | ILE | C | 131 | 21001 | 19849 | 14913 | −5669 | −363 | 1863 | C |
| ATOM | 931 | N | ALA | C | 132 | 60.733 | 8.098 | 10.044 | 1.00 | 136.61 | | N |
| ANISOU | 931 | N | ALA | C | 132 | 18791 | 20041 | 13072 | −5658 | −134 | 955 | N |
| ATOM | 932 | CA | ALA | C | 132 | 59.977 | 9.152 | 10.725 | 1.00 | 136.34 | | C |
| ANISOU | 932 | CA | ALA | C | 132 | 18516 | 20438 | 12849 | −5666 | −74 | 661 | C |
| ATOM | 933 | C | ALA | C | 132 | 60.543 | 10.523 | 10.349 | 1.00 | 136.51 | | C |
| ANISOU | 933 | C | ALA | C | 132 | 18367 | 20350 | 13150 | −5318 | −115 | 449 | C |
| ATOM | 934 | O | ALA | C | 132 | 59.815 | 11.353 | 9.800 | 1.00 | 134.74 | | O |
| ANISOU | 934 | O | ALA | C | 132 | 17961 | 20255 | 12977 | −5190 | −27 | 192 | O |
| ATOM | 935 | CB | ALA | C | 132 | 60.033 | 8.947 | 12.233 | 1.00 | 139.71 | | C |
| ANISOU | 935 | CB | ALA | C | 132 | 18985 | 21153 | 12946 | −5883 | −144 | 727 | C |
| ATOM | 936 | N | MET | C | 133 | 61.861 | 10.716 | 10.564 | 1.00 | 131.95 | | N |
| ANISOU | 936 | N | MET | C | 133 | 17860 | 19509 | 12766 | −5164 | −256 | 568 | N |
| ATOM | 937 | CA | MET | C | 133 | 62.581 | 11.952 | 10.237 | 1.00 | 130.20 | | C |
| ANISOU | 937 | CA | MET | C | 133 | 17506 | 19133 | 12830 | −4870 | −315 | 412 | C |
| ATOM | 938 | C | MET | C | 133 | 62.801 | 12.120 | 8.723 | 1.00 | 129.08 | | C |
| ANISOU | 938 | C | MET | C | 133 | 17360 | 18665 | 13020 | −4665 | −258 | 431 | C |
| ATOM | 939 | O | MET | C | 133 | 62.972 | 13.247 | 8.252 | 1.00 | 127.16 | | O |
| ANISOU | 939 | O | MET | C | 133 | 16987 | 18346 | 12982 | −4452 | −260 | 261 | O |
| ATOM | 940 | CB | MET | C | 133 | 63.898 | 12.109 | 11.037 | 1.00 | 133.66 | | C |
| ANISOU | 940 | CB | MET | C | 133 | 17992 | 19452 | 13338 | −4805 | −485 | 515 | C |
| ATOM | 941 | CG | MET | C | 133 | 64.433 | 10.820 | 11.659 | 1.00 | 139.36 | | C |
| ANISOU | 941 | CG | MET | C | 133 | 18934 | 20099 | 13917 | −4975 | −574 | 818 | C |
| ATOM | 942 | SD | MET | C | 133 | 64.461 | 10.828 | 13.477 | 1.00 | 146.97 | | S |
| ANISOU | 942 | SD | MET | C | 133 | 19906 | 21415 | 14521 | −5147 | −689 | 812 | S |
| ATOM | 943 | CE | MET | C | 133 | 66.129 | 11.417 | 13.781 | 1.00 | 143.09 | | C |
| ANISOU | 943 | CE | MET | C | 133 | 19387 | 20676 | 14306 | −4899 | −879 | 835 | C |
| ATOM | 944 | N | ALA | C | 134 | 62.736 | 11.009 | 7.957 | 1.00 | 123.53 | | N |
| ANISOU | 944 | N | ALA | C | 134 | 16803 | 17778 | 12355 | −4735 | −209 | 629 | N |
| ATOM | 945 | CA | ALA | C | 134 | 62.833 | 11.010 | 6.495 | 1.00 | 120.32 | | C |
| ANISOU | 945 | CA | ALA | C | 134 | 16395 | 17106 | 12215 | −4560 | −143 | 651 | C |
| ATOM | 946 | C | ALA | C | 134 | 61.508 | 11.549 | 5.925 | 1.00 | 122.34 | | C |
| ANISOU | 946 | C | ALA | C | 134 | 16507 | 17569 | 12406 | −4538 | −5 | 414 | C |
| ATOM | 947 | O | ALA | C | 134 | 61.050 | 11.119 | 4.857 | 1.00 | 120.49 | | O |
| ANISOU | 947 | O | ALA | C | 134 | 16297 | 17234 | 12252 | −4503 | 82 | 436 | O |
| ATOM | 948 | CB | ALA | C | 134 | 63.106 | 9.603 | 5.983 | 1.00 | 120.92 | | C |
| ANISOU | 948 | CB | ALA | C | 134 | 16674 | 16958 | 12314 | −4648 | −150 | 903 | C |
| ATOM | 949 | N | GLU | C | 135 | 60.888 | 12.491 | 6.682 | 1.00 | 118.84 | | N |
| ANISOU | 949 | N | GLU | C | 135 | 15909 | 17437 | 11808 | −4546 | 1 | 169 | N |
| ATOM | 950 | CA | GLU | C | 135 | 59.672 | 13.216 | 6.346 | 1.00 | 117.95 | | C |
| ANISOU | 950 | CA | GLU | C | 135 | 15630 | 17572 | 11615 | −4484 | 99 | −110 | C |
| ATOM | 951 | C | GLU | C | 135 | 60.045 | 14.098 | 5.170 | 1.00 | 116.51 | | C |
| ANISOU | 951 | C | GLU | C | 135 | 15399 | 17120 | 11749 | −4190 | 100 | −173 | C |
| ATOM | 952 | O | GLU | C | 135 | 59.417 | 13.977 | 4.109 | 1.00 | 115.55 | | O |
| ANISOU | 952 | O | GLU | C | 135 | 15261 | 16963 | 11679 | −4125 | 192 | −205 | O |
| ATOM | 953 | CB | GLU | C | 135 | 59.180 | 14.046 | 7.538 | 1.00 | 121.28 | | C |
| ANISOU | 953 | CB | GLU | C | 135 | 15902 | 18363 | 11817 | −4518 | 65 | −361 | C |
| ATOM | 954 | CG | GLU | C | 135 | 57.696 | 13.859 | 7.813 | 1.00 | 135.75 | | C |
| ANISOU | 954 | CG | GLU | C | 135 | 17614 | 20634 | 13330 | −4685 | 179 | −551 | C |
| ATOM | 955 | CD | GLU | C | 135 | 56.748 | 14.527 | 6.832 | 1.00 | 157.13 | | C |
| ANISOU | 955 | CD | GLU | C | 135 | 20175 | 23429 | 16100 | −4498 | 259 | −803 | C |
| ATOM | 956 | OE1 | GLU | C | 135 | 56.147 | 15.561 | 7.202 | 1.00 | 152.00 | | O |
| ANISOU | 956 | OE1 | GLU | C | 135 | 19348 | 23058 | 15348 | −4381 | 244 | −1115 | O |
| ATOM | 957 | OE2 | GLU | C | 135 | 56.593 | 14.010 | 5.702 | 1.00 | 148.87 | | O |
| ANISOU | 957 | OE2 | GLU | C | 135 | 19188 | 22182 | 15192 | −4454 | 326 | −702 | O |
| ATOM | 958 | N | ASP | C | 136 | 61.137 | 14.904 | 5.322 | 1.00 | 108.61 | | N |
| ANISOU | 958 | N | ASP | C | 136 | 14390 | 15915 | 10962 | −4030 | −7 | −164 | N |
| ATOM | 959 | CA | ASP | C | 136 | 61.714 | 15.663 | 4.212 | 1.00 | 104.74 | | C |
| ANISOU | 959 | CA | ASP | C | 136 | 13885 | 15127 | 10784 | −3789 | −14 | −158 | C |
| ATOM | 960 | C | ASP | C | 136 | 62.829 | 14.789 | 3.626 | 1.00 | 102.40 | | C |
| ANISOU | 960 | C | ASP | C | 136 | 13716 | 14524 | 10666 | −3791 | −27 | 137 | C |
| ATOM | 961 | O | ASP | C | 136 | 63.804 | 14.461 | 4.309 | 1.00 | 102.17 | | O |
| ANISOU | 961 | O | ASP | C | 136 | 13745 | 14407 | 10670 | −3845 | −121 | 274 | O |
| ATOM | 962 | CB | ASP | C | 136 | 62.232 | 17.053 | 4.617 | 1.00 | 106.56 | | C |
| ANISOU | 962 | CB | ASP | C | 136 | 14037 | 15297 | 11156 | −3640 | −122 | −312 | C |
| ATOM | 963 | CG | ASP | C | 136 | 63.005 | 17.768 | 3.513 | 1.00 | 109.34 | | C |
| ANISOU | 963 | CG | ASP | C | 136 | 14401 | 15310 | 11834 | −3442 | −138 | −245 | C |
| ATOM | 964 | OD1 | ASP | C | 136 | 62.579 | 17.684 | 2.331 | 1.00 | 107.94 | | O |
| ANISOU | 964 | OD1 | ASP | C | 136 | 14236 | 15046 | 11730 | −3345 | −48 | −221 | O |
| ATOM | 965 | OD2 | ASP | C | 136 | 64.023 | 18.418 | 3.830 | 1.00 | 111.98 | | O |
| ANISOU | 965 | OD2 | ASP | C | 136 | 14729 | 15481 | 12338 | −3395 | −239 | −218 | O |
| ATOM | 966 | N | VAL | C | 137 | 62.633 | 14.372 | 2.374 | 1.00 | 93.83 | | N |
| ANISOU | 966 | N | VAL | C | 137 | 12667 | 13308 | 9676 | −3722 | 63 | 214 | N |
| ATOM | 967 | CA | VAL | C | 137 | 63.500 | 13.468 | 1.624 | 1.00 | 90.78 | | C |
| ANISOU | 967 | CA | VAL | C | 137 | 12388 | 12665 | 9440 | −3697 | 66 | 455 | C |
| ATOM | 968 | C | VAL | C | 137 | 64.956 | 13.956 | 1.523 | 1.00 | 90.01 | | C |
| ANISOU | 968 | C | VAL | C | 137 | 12281 | 12331 | 9588 | −3573 | −21 | 566 | C |
| ATOM | 969 | O | VAL | C | 137 | 65.867 | 13.140 | 1.679 | 1.00 | 89.71 | | O |
| ANISOU | 969 | O | VAL | C | 137 | 12324 | 12168 | 9596 | −3607 | −81 | 748 | O |
| ATOM | 970 | CB | VAL | C | 137 | 62.883 | 13.140 | 0.241 | 1.00 | 93.21 | | C |
| ANISOU | 970 | CB | VAL | C | 137 | 12702 | 12915 | 9800 | −3611 | 181 | 455 | C |
| ATOM | 971 | CG1 | VAL | C | 137 | 62.757 | 14.371 | −0.657 | 1.00 | 92.18 | | C |
| ANISOU | 971 | CG1 | VAL | C | 137 | 12472 | 12729 | 9823 | −3398 | 218 | 330 | C |
| ATOM | 972 | CG2 | VAL | C | 137 | 63.621 | 12.011 | −0.460 | 1.00 | 92.34 | | C |
| ANISOU | 972 | CG2 | VAL | C | 137 | 12703 | 12580 | 9801 | −3593 | 181 | 675 | C |
| ATOM | 973 | N | ARG | C | 138 | 65.165 | 15.273 | 1.306 | 1.00 | 82.86 | | N |
| ANISOU | 973 | N | ARG | C | 138 | 11278 | 11370 | 8833 | −3439 | −39 | 451 | N |
| ATOM | 974 | CA | ARG | C | 138 | 66.483 | 15.900 | 1.177 | 1.00 | 80.78 | | C |
| ANISOU | 974 | CA | ARG | C | 138 | 10980 | 10905 | 8807 | −3348 | −112 | 532 | C |
| ATOM | 975 | C | ARG | C | 138 | 67.460 | 15.466 | 2.274 | 1.00 | 80.79 | | C |
| ANISOU | 975 | C | ARG | C | 138 | 11006 | 10906 | 8785 | −3443 | −231 | 627 | C |
| ATOM | 976 | O | ARG | C | 138 | 68.655 | 15.390 | 2.010 | 1.00 | 80.06 | | O |
| ANISOU | 976 | O | ARG | C | 138 | 10900 | 10650 | 8868 | −3388 | −278 | 760 | O |
| ATOM | 977 | CB | ARG | C | 138 | 66.368 | 17.436 | 1.092 | 1.00 | 81.76 | | C |
| ANISOU | 977 | CB | ARG | C | 138 | 11021 | 10996 | 9047 | −3246 | −140 | 361 | C |
| ATOM | 978 | CG | ARG | C | 138 | 65.936 | 17.977 | −0.274 | 1.00 | 88.24 | | C |
| ANISOU | 978 | CG | ARG | C | 138 | 11833 | 11703 | 9992 | −3096 | −52 | 348 | C |
| ATOM | 979 | CD | ARG | C | 138 | 64.446 | 18.270 | −0.352 | 1.00 | 105.08 | | C |
| ANISOU | 979 | CD | ARG | C | 138 | 13950 | 14017 | 11959 | −3054 | 5 | 148 | C |
| ATOM | 980 | NE | ARG | C | 138 | 63.914 | 18.016 | −1.698 | 1.00 | 123.64 | | N |
| ANISOU | 980 | NE | ARG | C | 138 | 16322 | 16315 | 14339 | −2954 | 116 | 206 | N |
| ATOM | 981 | CZ | ARG | C | 138 | 62.640 | 17.739 | −1.977 | 1.00 | 138.12 | | C |
| ANISOU | 981 | CZ | ARG | C | 138 | 18143 | 18327 | 16010 | −2934 | 190 | 78 | C |
| ATOM | 982 | NH1 | ARG | C | 138 | 61.738 | 17.665 | −1.002 | 1.00 | 130.60 | | N |
| ANISOU | 982 | NH1 | ARG | C | 138 | 17144 | 17634 | 14843 | −3019 | 176 | −114 | N |
| ATOM | 983 | NH2 | ARG | C | 138 | 62.262 | 17.519 | −3.230 | 1.00 | 115.19 | | N |
| ANISOU | 983 | NH2 | ARG | C | 138 | 15255 | 15369 | 13142 | −2833 | 280 | 134 | N |
| ATOM | 984 | N | ILE | C | 139 | 66.942 | 15.113 | 3.471 | 1.00 | 75.64 | | N |
| ANISOU | 984 | N | ILE | C | 139 | 10384 | 10459 | 7897 | −3585 | −279 | 564 | N |
| ATOM | 985 | CA | ILE | C | 139 | 67.722 | 14.619 | 4.618 | 1.00 | 75.49 | | C |
| ANISOU | 985 | CA | ILE | C | 139 | 10407 | 10476 | 7799 | −3681 | −402 | 655 | C |
| ATOM | 986 | C | ILE | C | 139 | 68.418 | 13.274 | 4.307 | 1.00 | 79.77 | | C |
| ANISOU | 986 | C | ILE | C | 139 | 11069 | 10875 | 8364 | −3693 | −424 | 894 | C |
| ATOM | 987 | O | ILE | C | 139 | 69.596 | 13.110 | 4.626 | 1.00 | 80.20 | | O |
| ANISOU | 987 | O | ILE | C | 139 | 11123 | 10827 | 8521 | −3651 | −531 | 999 | O |
| ATOM | 988 | CB | ILE | C | 139 | 66.863 | 14.569 | 5.916 | 1.00 | 78.83 | | C |
| ANISOU | 988 | CB | ILE | C | 139 | 10837 | 11192 | 7925 | −3839 | −432 | 530 | C |
| ATOM | 989 | CG1 | ILE | C | 139 | 66.518 | 15.981 | 6.385 | 1.00 | 79.38 | | C |
| ANISOU | 989 | CG1 | ILE | C | 139 | 10773 | 11382 | 8004 | −3781 | −462 | 270 | C |
| ATOM | 990 | CG2 | ILE | C | 139 | 67.571 | 13.818 | 7.040 | 1.00 | 80.07 | | C |
| ANISOU | 990 | CG2 | ILE | C | 139 | 11075 | 11394 | 7952 | −3950 | −556 | 666 | C |
| ATOM | 991 | CD1 | ILE | C | 139 | 65.129 | 16.223 | 6.608 | 1.00 | 85.07 | | C |
| ANISOU | 991 | CD1 | ILE | C | 139 | 11448 | 12371 | 8504 | −3835 | −391 | 70 | C |
| ATOM | 992 | N | ILE | C | 140 | 67.696 | 12.330 | 3.672 | 1.00 | 75.63 | | N |
| ANISOU | 992 | N | ILE | C | 140 | 10642 | 10342 | 7751 | −3738 | −336 | 962 | N |
| ATOM | 993 | CA | ILE | C | 140 | 68.235 | 11.010 | 3.327 | 1.00 | 74.84 | | C |
| ANISOU | 993 | CA | ILE | C | 140 | 10681 | 10084 | 7672 | −3736 | −372 | 1166 | C |
| ATOM | 994 | C | ILE | C | 140 | 69.200 | 11.075 | 2.168 | 1.00 | 74.23 | | C |
| ANISOU | 994 | C | ILE | C | 140 | 10550 | 9796 | 7859 | −3542 | −358 | 1241 | C |
| ATOM | 995 | O | ILE | C | 140 | 70.191 | 10.352 | 2.174 | 1.00 | 74.27 | | O |
| ANISOU | 995 | O | ILE | C | 140 | 10609 | 9676 | 7936 | −3481 | −453 | 1377 | O |
| ATOM | 996 | CB | ILE | C | 140 | 67.168 | 9.928 | 3.103 | 1.00 | 78.55 | | C |
| ANISOU | 996 | CB | ILE | C | 140 | 11285 | 10597 | 7964 | −3869 | −300 | 1209 | C |
| ATOM | 997 | CG1 | ILE | C | 140 | 65.785 | 10.380 | 3.572 | 1.00 | 80.52 | | C |
| ANISOU | 997 | CG1 | ILE | C | 140 | 11481 | 11118 | 7995 | −4019 | −215 | 1037 | C |
| ATOM | 998 | CG2 | ILE | C | 140 | 67.589 | 8.653 | 3.807 | 1.00 | 80.16 | | C |
| ANISOU | 998 | CG2 | ILE | C | 140 | 11681 | 10723 | 8052 | −3976 | −419 | 1398 | C |
| ATOM | 999 | CD1 | ILE | C | 140 | 64.679 | 10.053 | 2.612 | 1.00 | 95.15 | | C |
| ANISOU | 999 | CD1 | ILE | C | 140 | 13340 | 13014 | 9801 | −4046 | −79 | 975 | C |
| ATOM | 1000 | N | ILE | C | 141 | 68.916 | 11.940 | 1.183 | 1.00 | 66.88 | | N |
| ANISOU | 1000 | N | ILE | C | 141 | 9512 | 8841 | 7061 | −3438 | −248 | 1150 | N |
| ATOM | 1001 | CA | ILE | C | 141 | 69.760 | 12.155 | 0.009 | 1.00 | 64.50 | | C |
| ANISOU | 1001 | CA | ILE | C | 141 | 9136 | 8381 | 6991 | −3269 | −210 | 1215 | C |
| ATOM | 1002 | C | ILE | C | 141 | 71.178 | 12.561 | 0.462 | 1.00 | 68.69 | | C |
| ANISOU | 1002 | C | ILE | C | 141 | 9581 | 8851 | 7668 | −3219 | −322 | 1269 | C |
| ATOM | 1003 | O | ILE | C | 141 | 72.156 | 11.960 | 0.008 | 1.00 | 68.42 | | O |
| ANISOU | 1003 | O | ILE | C | 141 | 9539 | 8718 | 7740 | −3122 | −358 | 1383 | O |
| ATOM | 1004 | CB | ILE | C | 141 | 69.092 | 13.180 | −0.938 | 1.00 | 65.72 | | C |
| ANISOU | 1004 | CB | ILE | C | 141 | 9201 | 8545 | 7223 | −3193 | −87 | 1105 | C |
| ATOM | 1005 | CG1 | ILE | C | 141 | 67.818 | 12.588 | −1.541 | 1.00 | 64.18 | | C |
| ANISOU | 1005 | CG1 | ILE | C | 141 | 9076 | 8422 | 6889 | −3220 | 17 | 1053 | C |
| ATOM | 1006 | CG2 | ILE | C | 141 | 70.051 | 13.628 | −2.047 | 1.00 | 66.79 | | C |
| ANISOU | 1006 | CG2 | ILE | C | 141 | 9247 | 8545 | 7585 | −3046 | −45 | 1183 | C |
| ATOM | 1007 | CD1 | ILE | C | 141 | 66.813 | 13.560 | −1.887 | 1.00 | 68.01 | | C |
| ANISOU | 1007 | CD1 | ILE | C | 141 | 9493 | 8987 | 7361 | −3175 | 101 | 899 | C |
| ATOM | 1008 | N | VAL | C | 142 | 71.268 | 13.528 | 1.406 | 1.00 | 65.16 | | N |
| ANISOU | 1008 | N | VAL | C | 142 | 9064 | 8482 | 7212 | −3283 | −386 | 1168 | N |
| ATOM | 1009 | CA | VAL | C | 142 | 72.515 | 14.011 | 2.008 | 1.00 | 65.10 | | C |
| ANISOU | 1009 | CA | VAL | C | 142 | 8960 | 8449 | 7324 | −3267 | −504 | 1183 | C |
| ATOM | 1010 | C | VAL | C | 142 | 73.167 | 12.848 | 2.774 | 1.00 | 69.05 | | C |
| ANISOU | 1010 | C | VAL | C | 142 | 9548 | 8961 | 7728 | −3285 | −635 | 1299 | C |
| ATOM | 1011 | O | VAL | C | 142 | 74.361 | 12.610 | 2.589 | 1.00 | 68.76 | | O |
| ANISOU | 1011 | O | VAL | C | 142 | 9448 | 8856 | 7821 | −3194 | −706 | 1379 | O |
| ATOM | 1012 | CB | VAL | C | 142 | 72.281 | 15.261 | 2.908 | 1.00 | 69.31 | | C |
| ANISOU | 1012 | CB | VAL | C | 142 | 9419 | 9069 | 7846 | −3338 | −555 | 1016 | C |
| ATOM | 1013 | CG1 | VAL | C | 142 | 73.526 | 15.621 | 3.702 | 1.00 | 70.51 | | C |
| ANISOU | 1013 | CG1 | VAL | C | 142 | 9477 | 9216 | 8099 | −3346 | −695 | 1019 | C |
| ATOM | 1014 | CG2 | VAL | C | 142 | 71.831 | 16.458 | 2.087 | 1.00 | 68.28 | | C |
| ANISOU | 1014 | CG2 | VAL | C | 142 | 9222 | 8876 | 7844 | −3289 | −460 | 914 | C |
| ATOM | 1015 | N | ALA | C | 143 | 72.368 | 12.093 | 3.581 | 1.00 | 66.12 | | N |
| ANISOU | 1015 | N | ALA | C | 143 | 9324 | 8680 | 7119 | −3401 | −667 | 1311 | N |
| ATOM | 1016 | CA | ALA | C | 143 | 72.839 | 10.938 | 4.383 | 1.00 | 67.18 | | C |
| ANISOU | 1016 | CA | ALA | C | 143 | 9594 | 8809 | 7124 | −3434 | −808 | 1441 | C |
| ATOM | 1017 | C | ALA | C | 143 | 73.460 | 9.858 | 3.499 | 1.00 | 70.40 | | C |
| ANISOU | 1017 | C | ALA | C | 143 | 10074 | 9045 | 7629 | −3302 | −827 | 1583 | C |
| ATOM | 1018 | O | ALA | C | 143 | 74.439 | 9.211 | 3.888 | 1.00 | 70.68 | | O |
| ANISOU | 1018 | O | ALA | C | 143 | 10154 | 9025 | 7677 | −3229 | −973 | 1679 | O |
| ATOM | 1019 | CB | ALA | C | 143 | 71.701 | 10.354 | 5.227 | 1.00 | 68.26 | | C |
| ANISOU | 1019 | CB | ALA | C | 143 | 9886 | 9075 | 6975 | −3618 | −807 | 1444 | C |
| ATOM | 1020 | N | LEU | C | 144 | 72.904 | 9.713 | 2.289 | 1.00 | 65.26 | | N |
| ANISOU | 1020 | N | LEU | C | 144 | 9426 | 8324 | 7048 | −3249 | −687 | 1576 | N |
| ATOM | 1021 | CA | LEU | C | 144 | 73.344 | 8.761 | 1.287 | 1.00 | 64.67 | | C |
| ANISOU | 1021 | CA | LEU | C | 144 | 9404 | 8101 | 7065 | −3107 | −685 | 1671 | C |
| ATOM | 1022 | C | LEU | C | 144 | 74.624 | 9.238 | 0.604 | 1.00 | 68.23 | | C |
| ANISOU | 1022 | C | LEU | C | 144 | 9667 | 8514 | 7743 | −2929 | −691 | 1673 | C |
| ATOM | 1023 | O | LEU | C | 144 | 75.549 | 8.440 | 0.435 | 1.00 | 68.73 | | O |
| ANISOU | 1023 | O | LEU | C | 144 | 9746 | 8504 | 7864 | −2794 | −793 | 1747 | O |
| ATOM | 1024 | CB | LEU | C | 144 | 72.212 | 8.489 | 0.278 | 1.00 | 63.29 | | C |
| ANISOU | 1024 | CB | LEU | C | 144 | 9290 | 7902 | 6857 | −3127 | −532 | 1640 | C |
| ATOM | 1025 | CG | LEU | C | 144 | 71.078 | 7.602 | 0.786 | 1.00 | 67.67 | | C |
| ANISOU | 1025 | CG | LEU | C | 144 | 10047 | 8475 | 7188 | −3304 | −538 | 1669 | C |
| ATOM | 1026 | CD1 | LEU | C | 144 | 69.846 | 7.796 | −0.028 | 1.00 | 66.23 | | C |
| ANISOU | 1026 | CD1 | LEU | C | 144 | 9850 | 8351 | 6963 | −3355 | −373 | 1576 | C |
| ATOM | 1027 | CD2 | LEU | C | 144 | 71.482 | 6.141 | 0.810 | 1.00 | 71.00 | | C |
| ANISOU | 1027 | CD2 | LEU | C | 144 | 10668 | 8730 | 7579 | −3265 | −664 | 1807 | C |
| ATOM | 1028 | N | ALA | C | 145 | 74.693 | 10.540 | 0.246 | 1.00 | 63.65 | | N |
| ANISOU | 1028 | N | ALA | C | 145 | 8909 | 7989 | 7286 | −2933 | −592 | 1589 | N |
| ATOM | 1029 | CA | ALA | C | 145 | 75.874 | 11.145 | −0.378 | 1.00 | 63.50 | | C |
| ANISOU | 1029 | CA | ALA | C | 145 | 8693 | 7963 | 7472 | −2818 | −580 | 1596 | C |
| ATOM | 1030 | C | ALA | C | 145 | 77.041 | 11.141 | 0.613 | 1.00 | 68.97 | | C |
| ANISOU | 1030 | C | ALA | C | 145 | 9313 | 8698 | 8195 | −2805 | −750 | 1610 | C |
| ATOM | 1031 | O | ALA | C | 145 | 78.187 | 10.997 | 0.200 | 1.00 | 70.52 | | O |
| ANISOU | 1031 | O | ALA | C | 145 | 9381 | 8899 | 8516 | −2679 | −791 | 1643 | O |
| ATOM | 1032 | CB | ALA | C | 145 | 75.566 | 12.562 | −0.832 | 1.00 | 63.34 | | C |
| ANISOU | 1032 | CB | ALA | C | 145 | 8545 | 7966 | 7554 | −2872 | −459 | 1519 | C |
| ATOM | 1033 | N | ASP | C | 146 | 76.739 | 11.259 | 1.920 | 1.00 | 64.72 | | N |
| ANISOU | 1033 | N | ASP | C | 146 | 8847 | 8220 | 7524 | −2927 | −851 | 1575 | N |
| ATOM | 1034 | CA | ASP | C | 146 | 77.716 | 11.215 | 3.005 | 1.00 | 65.75 | | C |
| ANISOU | 1034 | CA | ASP | C | 146 | 8929 | 8410 | 7641 | −2921 | −1030 | 1578 | C |
| ATOM | 1035 | C | ASP | C | 146 | 78.307 | 9.795 | 3.052 | 1.00 | 69.47 | | C |
| ANISOU | 1035 | C | ASP | C | 146 | 9519 | 8817 | 8059 | −2786 | −1162 | 1691 | C |
| ATOM | 1036 | O | ASP | C | 146 | 79.529 | 9.621 | 2.970 | 1.00 | 69.30 | | O |
| ANISOU | 1036 | O | ASP | C | 146 | 9373 | 8812 | 8146 | −2648 | −1260 | 1705 | O |
| ATOM | 1037 | CB | ASP | C | 146 | 77.016 | 11.593 | 4.331 | 1.00 | 68.17 | | C |
| ANISOU | 1037 | CB | ASP | C | 146 | 9317 | 8815 | 7769 | −3085 | −1092 | 1511 | C |
| ATOM | 1038 | CG | ASP | C | 146 | 77.826 | 11.407 | 5.600 | 1.00 | 83.89 | | C |
| ANISOU | 1038 | CG | ASP | C | 146 | 11303 | 10889 | 9683 | −3089 | −1291 | 1519 | C |
| ATOM | 1039 | OD1 | ASP | C | 146 | 78.960 | 11.936 | 5.666 | 1.00 | 85.95 | | O |
| ANISOU | 1039 | OD1 | ASP | C | 146 | 11379 | 11184 | 10094 | −3029 | −1362 | 1476 | O |
| ATOM | 1040 | OD2 | ASP | C | 146 | 77.299 | 10.789 | 6.556 | 1.00 | 91.49 | | O |
| ANISOU | 1040 | OD2 | ASP | C | 146 | 12441 | 11901 | 10422 | −3170 | −1375 | 1563 | O |
| ATOM | 1041 | N | ARG | C | 147 | 77.404 | 8.786 | 3.101 | 1.00 | 65.39 | | N |
| ANISOU | 1041 | N | ARG | C | 147 | 9240 | 8224 | 7382 | −2825 | −1164 | 1764 | N |
| ATOM | 1042 | CA | ARG | C | 147 | 77.697 | 7.357 | 3.138 | 1.00 | 65.50 | | C |
| ANISOU | 1042 | CA | ARG | C | 147 | 9441 | 8120 | 7325 | −2717 | −1298 | 1878 | C |
| ATOM | 1043 | C | ARG | C | 147 | 78.500 | 6.898 | 1.922 | 1.00 | 69.66 | | C |
| ANISOU | 1043 | C | ARG | C | 147 | 9877 | 8567 | 8023 | −2489 | −1283 | 1887 | C |
| ATOM | 1044 | O | ARG | C | 147 | 79.474 | 6.171 | 2.095 | 1.00 | 71.48 | | O |
| ANISOU | 1044 | O | ARG | C | 147 | 10131 | 8752 | 8276 | −2321 | −1451 | 1930 | O |
| ATOM | 1045 | CB | ARG | C | 147 | 76.394 | 6.555 | 3.279 | 1.00 | 62.54 | | C |
| ANISOU | 1045 | CB | ARG | C | 147 | 9329 | 7673 | 6760 | −2861 | −1261 | 1941 | C |
| ATOM | 1046 | CG | ARG | C | 147 | 76.567 | 5.045 | 3.442 | 1.00 | 71.22 | | C |
| ANISOU | 1046 | CG | ARG | C | 147 | 10685 | 8605 | 7768 | −2791 | −1426 | 2076 | C |
| ATOM | 1047 | CD | ARG | C | 147 | 77.642 | 4.644 | 4.439 | 1.00 | 79.25 | | C |
| ANISOU | 1047 | CD | ARG | C | 147 | 11744 | 9622 | 8745 | −2696 | −1664 | 2142 | C |
| ATOM | 1048 | NE | ARG | C | 147 | 77.343 | 5.040 | 5.811 | 1.00 | 81.35 | | N |
| ANISOU | 1048 | NE | ARG | C | 147 | 12061 | 10024 | 8823 | −2885 | −1728 | 2158 | N |
| ATOM | 1049 | CZ | ARG | C | 147 | 76.886 | 4.208 | 6.735 | 1.00 | 88.95 | | C |
| ANISOU | 1049 | CZ | ARG | C | 147 | 13290 | 10943 | 9562 | −3004 | −1859 | 2289 | C |
| ATOM | 1050 | NH1 | ARG | C | 147 | 76.661 | 2.934 | 6.435 | 1.00 | 74.00 | | N |
| ANISOU | 1050 | NH1 | ARG | C | 147 | 11658 | 8836 | 7624 | −2961 | −1949 | 2419 | N |
| ATOM | 1051 | NH2 | ARG | C | 147 | 76.642 | 4.642 | 7.965 | 1.00 | 72.28 | | N |
| ANISOU | 1051 | NH2 | ARG | C | 147 | 11196 | 9002 | 7264 | −3172 | −1905 | 2290 | N |
| ATOM | 1052 | N | LEU | C | 148 | 78.102 | 7.323 | 0.707 | 1.00 | 63.94 | | N |
| ANISOU | 1052 | N | LEU | C | 148 | 9046 | 7841 | 7406 | −2469 | −1091 | 1839 | N |
| ATOM | 1053 | CA | LEU | C | 148 | 78.795 | 6.978 | −0.537 | 1.00 | 63.29 | | C |
| ANISOU | 1053 | CA | LEU | C | 148 | 8848 | 7730 | 7468 | −2260 | −1048 | 1831 | C |
| ATOM | 1054 | C | LEU | C | 148 | 80.245 | 7.467 | −0.516 | 1.00 | 69.68 | | C |
| ANISOU | 1054 | C | LEU | C | 148 | 9399 | 8657 | 8418 | −2139 | −1114 | 1795 | C |
| ATOM | 1055 | O | LEU | C | 148 | 81.145 | 6.723 | −0.918 | 1.00 | 70.95 | | O |
| ANISOU | 1055 | O | LEU | C | 148 | 9507 | 8814 | 8637 | −1926 | −1205 | 1797 | O |
| ATOM | 1056 | CB | LEU | C | 148 | 78.037 | 7.536 | −1.763 | 1.00 | 60.73 | | C |
| ANISOU | 1056 | CB | LEU | C | 148 | 8456 | 7417 | 7202 | −2289 | −821 | 1791 | C |
| ATOM | 1057 | CG | LEU | C | 148 | 78.591 | 7.240 | −3.172 | 1.00 | 63.33 | | C |
| ANISOU | 1057 | CG | LEU | C | 148 | 8661 | 7753 | 7650 | −2087 | −741 | 1777 | C |
| ATOM | 1058 | CD1 | LEU | C | 148 | 78.795 | 5.756 | −3.401 | 1.00 | 64.18 | | C |
| ANISOU | 1058 | CD1 | LEU | C | 148 | 8928 | 7739 | 7720 | −1908 | −872 | 1802 | C |
| ATOM | 1059 | CD2 | LEU | C | 148 | 77.664 | 7.778 | −4.229 | 1.00 | 60.91 | | C |
| ANISOU | 1059 | CD2 | LEU | C | 148 | 8335 | 7455 | 7353 | −2138 | −532 | 1749 | C |
| ATOM | 1060 | N | HIS | C | 149 | 80.467 | 8.694 | −0.003 | 1.00 | 65.87 | | N |
| ANISOU | 1060 | N | HIS | C | 149 | 8756 | 8285 | 7985 | −2275 | −1083 | 1748 | N |
| ATOM | 1061 | CA | HIS | C | 149 | 81.800 | 9.271 | 0.078 | 1.00 | 66.27 | | C |
| ANISOU | 1061 | CA | HIS | C | 149 | 8543 | 8466 | 8169 | −2212 | −1139 | 1704 | C |
| ATOM | 1062 | C | HIS | C | 149 | 82.672 | 8.591 | 1.110 | 1.00 | 74.24 | | C |
| ANISOU | 1062 | C | HIS | C | 149 | 9579 | 9507 | 9124 | −2107 | −1378 | 1713 | C |
| ATOM | 1063 | O | HIS | C | 149 | 83.857 | 8.378 | 0.846 | 1.00 | 75.71 | | O |
| ANISOU | 1063 | O | HIS | C | 149 | 9579 | 9785 | 9402 | −1938 | −1455 | 1682 | O |
| ATOM | 1064 | CB | HIS | C | 149 | 81.780 | 10.788 | 0.281 | 1.00 | 65.32 | | C |
| ANISOU | 1064 | CB | HIS | C | 149 | 8258 | 8426 | 8136 | −2399 | −1047 | 1644 | C |
| ATOM | 1065 | CG | HIS | C | 149 | 83.153 | 11.359 | 0.235 | 1.00 | 69.24 | | C |
| ANISOU | 1065 | CG | HIS | C | 149 | 8470 | 9061 | 8778 | −2364 | −1090 | 1601 | C |
| ATOM | 1066 | CD2 | HIS | C | 149 | 84.098 | 11.248 | −0.726 | 1.00 | 71.30 | | C |
| ANISOU | 1066 | CD2 | HIS | C | 149 | 8518 | 9417 | 9157 | −2240 | −1036 | 1600 | C |
| ATOM | 1067 | ND1 | HIS | C | 149 | 83.697 | 11.984 | 1.319 | 1.00 | 71.65 | | N |
| ANISOU | 1067 | ND1 | HIS | C | 149 | 8685 | 9446 | 9094 | −2453 | −1219 | 1543 | N |
| ATOM | 1068 | CE1 | HIS | C | 149 | 84.932 | 12.299 | 0.971 | 1.00 | 72.30 | | C |
| ANISOU | 1068 | CE1 | HIS | C | 149 | 8496 | 9662 | 9314 | −2403 | −1231 | 1508 | C |
| ATOM | 1069 | NE2 | HIS | C | 149 | 85.217 | 11.869 | −0.250 | 1.00 | 72.53 | | N |
| ANISOU | 1069 | NE2 | HIS | C | 149 | 8437 | 9718 | 9405 | −2278 | −1119 | 1543 | N |
| ATOM | 1070 | N | ASN | C | 150 | 82.102 | 8.244 | 2.277 | 1.00 | 72.37 | | N |
| ANISOU | 1070 | N | ASN | C | 150 | 9560 | 9216 | 8723 | −2202 | −1500 | 1751 | N |
| ATOM | 1071 | CA | ASN | C | 150 | 82.872 | 7.567 | 3.324 | 1.00 | 74.95 | | C |
| ANISOU | 1071 | CA | ASN | C | 150 | 9945 | 9564 | 8968 | −2099 | −1748 | 1778 | C |
| ATOM | 1072 | C | ASN | C | 150 | 83.251 | 6.144 | 2.899 | 1.00 | 81.77 | | C |
| ANISOU | 1072 | C | ASN | C | 150 | 10950 | 10306 | 9811 | −1854 | −1876 | 1839 | C |
| ATOM | 1073 | O | ASN | C | 150 | 84.332 | 5.676 | 3.256 | 1.00 | 83.71 | | O |
| ANISOU | 1073 | O | ASN | C | 150 | 11130 | 10600 | 10074 | −1664 | −2069 | 1824 | O |
| ATOM | 1074 | CB | ASN | C | 150 | 82.168 | 7.618 | 4.681 | 1.00 | 74.16 | | C |
| ANISOU | 1074 | CB | ASN | C | 150 | 10035 | 9466 | 8674 | −2277 | −1839 | 1812 | C |
| ATOM | 1075 | CG | ASN | C | 150 | 81.995 | 9.022 | 5.211 | 1.00 | 90.30 | | C |
| ANISOU | 1075 | CG | ASN | C | 150 | 11914 | 11645 | 10750 | −2468 | −1763 | 1710 | C |
| ATOM | 1076 | ND2 | ASN | C | 150 | 80.755 | 9.386 | 5.483 | 1.00 | 83.15 | | N |
| ANISOU | 1076 | ND2 | ASN | C | 150 | 11130 | 10723 | 9739 | −2656 | −1650 | 1700 | N |
| ATOM | 1077 | OD1 | ASN | C | 150 | 82.958 | 9.791 | 5.378 | 1.00 | 79.92 | | O |
| ANISOU | 1077 | OD1 | ASN | C | 150 | 10359 | 10452 | 9555 | −2448 | −1812 | 1627 | O |
| ATOM | 1078 | N | LEU | C | 151 | 82.390 | 5.495 | 2.080 | 1.00 | 77.98 | | N |
| ANISOU | 1078 | N | LEU | C | 151 | 10645 | 9675 | 9307 | −1842 | −1775 | 1886 | N |
| ATOM | 1079 | CA | LEU | C | 151 | 82.610 | 4.166 | 1.512 | 1.00 | 79.40 | | C |
| ANISOU | 1079 | CA | LEU | C | 151 | 10976 | 9705 | 9486 | −1611 | −1883 | 1922 | C |
| ATOM | 1080 | C | LEU | C | 151 | 83.705 | 4.288 | 0.462 | 1.00 | 86.64 | | C |
| ANISOU | 1080 | C | LEU | C | 151 | 11596 | 10745 | 10578 | −1379 | −1841 | 1823 | C |
| ATOM | 1081 | O | LEU | C | 151 | 84.554 | 3.403 | 0.357 | 1.00 | 88.92 | | O |
| ANISOU | 1081 | O | LEU | C | 151 | 11887 | 11010 | 10890 | −1113 | −2016 | 1799 | O |
| ATOM | 1082 | CB | LEU | C | 151 | 81.317 | 3.656 | 0.859 | 1.00 | 77.75 | | C |
| ANISOU | 1082 | CB | LEU | C | 151 | 10999 | 9327 | 9215 | −1705 | −1757 | 1971 | C |
| ATOM | 1083 | CG | LEU | C | 151 | 80.679 | 2.415 | 1.458 | 1.00 | 83.19 | | C |
| ANISOU | 1083 | CG | LEU | C | 151 | 12069 | 9800 | 9740 | −1745 | −1918 | 2089 | C |
| ATOM | 1084 | CD1 | LEU | C | 151 | 80.174 | 2.677 | 2.825 | 1.00 | 83.15 | | C |
| ANISOU | 1084 | CD1 | LEU | C | 151 | 12195 | 9836 | 9562 | −1995 | −1962 | 2162 | C |
| ATOM | 1085 | CD2 | LEU | C | 151 | 79.486 | 1.997 | 0.650 | 1.00 | 85.64 | | C |
| ANISOU | 1085 | CD2 | LEU | C | 151 | 12549 | 9967 | 10023 | −1824 | −1783 | 2105 | C |
| ATOM | 1086 | N | ARG | C | 152 | 83.708 | 5.410 | −0.285 | 1.00 | 82.86 | | N |
| ANISOU | 1086 | N | ARG | C | 152 | 10860 | 10411 | 10211 | −1480 | −1618 | 1763 | N |
| ATOM | 1087 | CA | ARG | C | 152 | 84.718 | 5.714 | −1.292 | 1.00 | 84.35 | | C |
| ANISOU | 1087 | CA | ARG | C | 152 | 10732 | 10771 | 10546 | −1322 | −1539 | 1679 | C |
| ATOM | 1088 | C | ARG | C | 152 | 86.077 | 6.028 | −0.637 | 1.00 | 93.37 | | C |
| ANISOU | 1088 | C | ARG | C | 152 | 11626 | 12104 | 11746 | −1239 | −1686 | 1615 | C |
| ATOM | 1089 | O | ARG | C | 152 | 87.115 | 5.905 | −1.293 | 1.00 | 94.82 | | O |
| ANISOU | 1089 | O | ARG | C | 152 | 11556 | 12450 | 12020 | −1048 | −1694 | 1536 | O |
| ATOM | 1090 | CB | ARG | C | 152 | 84.266 | 6.896 | −2.152 | 1.00 | 83.18 | | C |
| ANISOU | 1090 | CB | ARG | C | 152 | 10418 | 10707 | 10480 | −1501 | −1268 | 1666 | C |
| ATOM | 1091 | CG | ARG | C | 152 | 83.318 | 6.538 | −3.286 | 1.00 | 91.12 | | C |
| ANISOU | 1091 | CG | ARG | C | 152 | 11543 | 11611 | 11467 | −1476 | −1108 | 1685 | C |
| ATOM | 1092 | CD | ARG | C | 152 | 82.584 | 7.780 | −3.743 | 1.00 | 95.35 | | C |
| ANISOU | 1092 | CD | ARG | C | 152 | 12005 | 12183 | 12039 | −1697 | −880 | 1698 | C |
| ATOM | 1093 | NE | ARG | C | 152 | 81.977 | 7.612 | −5.061 | 1.00 | 97.49 | | N |
| ANISOU | 1093 | NE | ARG | C | 152 | 12287 | 12436 | 12318 | −1637 | −708 | 1697 | N |
| ATOM | 1094 | CZ | ARG | C | 152 | 81.442 | 8.600 | −5.773 | 1.00 | 103.03 | | C |
| ANISOU | 1094 | CZ | ARG | C | 152 | 12912 | 13180 | 13057 | −1767 | −507 | 1710 | C |
| ATOM | 1095 | NH1 | ARG | C | 152 | 81.440 | 9.843 | −5.302 | 1.00 | 79.96 | | N |
| ANISOU | 1095 | NH1 | ARG | C | 152 | 9904 | 10295 | 10182 | −1966 | −458 | 1719 | N |
| ATOM | 1096 | NH2 | ARG | C | 152 | 80.910 | 8.354 | −6.964 | 1.00 | 89.87 | | N |
| ANISOU | 1096 | NH2 | ARG | C | 152 | 11260 | 11509 | 11377 | −1689 | −368 | 1707 | N |
| ATOM | 1097 | N | THR | C | 153 | 86.063 | 6.442 | 0.650 | 1.00 | 92.21 | | N |
| ANISOU | 1097 | N | THR | C | 153 | 11535 | 11965 | 11536 | −1383 | −1801 | 1636 | N |
| ATOM | 1098 | CA | THR | C | 153 | 87.259 | 6.779 | 1.440 | 1.00 | 94.89 | | C |
| ANISOU | 1098 | CA | THR | C | 153 | 11655 | 12488 | 11911 | −1331 | −1959 | 1567 | C |
| ATOM | 1099 | C | THR | C | 153 | 87.438 | 5.801 | 2.636 | 1.00 | 102.02 | | C |
| ANISOU | 1099 | C | THR | C | 153 | 12791 | 13300 | 12670 | −1205 | −2250 | 1612 | C |
| ATOM | 1100 | O | THR | C | 153 | 87.953 | 6.184 | 3.696 | 1.00 | 102.54 | | O |
| ANISOU | 1100 | O | THR | C | 153 | 12786 | 13474 | 12700 | −1255 | −2387 | 1584 | O |
| ATOM | 1101 | CB | THR | C | 153 | 87.210 | 8.258 | 1.871 | 1.00 | 102.28 | | C |
| ANISOU | 1101 | CB | THR | C | 153 | 12410 | 13540 | 12910 | −1611 | −1844 | 1529 | C |
| ATOM | 1102 | CG2 | THR | C | 153 | 87.141 | 9.218 | 0.688 | 1.00 | 98.89 | | C |
| ANISOU | 1102 | CG2 | THR | C | 153 | 11763 | 13186 | 12623 | −1724 | −1587 | 1505 | C |
| ATOM | 1103 | OG1 | THR | C | 153 | 86.099 | 8.459 | 2.744 | 1.00 | 101.06 | | O |
| ANISOU | 1103 | OG1 | THR | C | 153 | 12512 | 13254 | 12632 | −1812 | −1842 | 1587 | O |
| ATOM | 1104 | N | LEU | C | 154 | 87.014 | 4.536 | 2.444 | 1.00 | 100.14 | | N |
| ANISOU | 1104 | N | LEU | C | 154 | 12844 | 12857 | 12349 | −1042 | −2352 | 1684 | N |
| ATOM | 1105 | CA | LEU | C | 154 | 87.040 | 3.504 | 3.476 | 1.00 | 102.52 | | C |
| ANISOU | 1105 | CA | LEU | C | 154 | 13439 | 13014 | 12499 | −932 | −2628 | 1767 | C |
| ATOM | 1106 | C | LEU | C | 154 | 88.359 | 2.754 | 3.597 | 1.00 | 113.92 | | C |
| ANISOU | 1106 | C | LEU | C | 154 | 14784 | 14529 | 13973 | −582 | −2886 | 1697 | C |
| ATOM | 1107 | O | LEU | C | 154 | 88.593 | 2.111 | 4.623 | 1.00 | 115.69 | | O |
| ANISOU | 1107 | O | LEU | C | 154 | 15209 | 14676 | 14073 | −487 | −3146 | 1759 | O |
| ATOM | 1108 | CB | LEU | C | 154 | 85.877 | 2.524 | 3.272 | 1.00 | 101.25 | | C |
| ANISOU | 1108 | CB | LEU | C | 154 | 13676 | 12563 | 12231 | −972 | −2624 | 1891 | C |
| ATOM | 1109 | CG | LEU | C | 154 | 85.237 | 1.960 | 4.540 | 1.00 | 105.84 | | C |
| ANISOU | 1109 | CG | LEU | C | 154 | 14622 | 12987 | 12604 | −1097 | −2798 | 2039 | C |
| ATOM | 1110 | CD1 | LEU | C | 154 | 84.722 | 3.061 | 5.444 | 1.00 | 104.30 | | C |
| ANISOU | 1110 | CD1 | LEU | C | 154 | 14375 | 12932 | 12323 | −1406 | −2700 | 2051 | C |
| ATOM | 1111 | CD2 | LEU | C | 154 | 84.117 | 1.018 | 4.203 | 1.00 | 107.16 | | C |
| ANISOU | 1111 | CD2 | LEU | C | 154 | 15151 | 12880 | 12684 | −1163 | −2776 | 2154 | C |
| ATOM | 1112 | N | GLU | C | 155 | 89.226 | 2.858 | 2.579 | 1.00 | 114.48 | | N |
| ANISOU | 1112 | N | GLU | C | 155 | 14537 | 14768 | 14190 | −386 | −2821 | 1564 | N |
| ATOM | 1113 | CA | GLU | C | 155 | 90.533 | 2.192 | 2.510 | 1.00 | 119.09 | | C |
| ANISOU | 1113 | CA | GLU | C | 155 | 14958 | 15477 | 14815 | −16 | −3046 | 1449 | C |
| ATOM | 1114 | C | GLU | C | 155 | 91.404 | 2.338 | 3.793 | 1.00 | 127.88 | | C |
| ANISOU | 1114 | C | GLU | C | 155 | 15995 | 16731 | 15864 | 48 | −3298 | 1420 | C |
| ATOM | 1115 | O | GLU | C | 155 | 92.058 | 1.370 | 4.190 | 1.00 | 129.72 | | O |
| ANISOU | 1115 | O | GLU | C | 155 | 16341 | 16908 | 16037 | 356 | −3588 | 1404 | O |
| ATOM | 1116 | CB | GLU | C | 155 | 91.304 | 2.648 | 1.243 | 1.00 | 120.91 | | C |
| ANISOU | 1116 | CB | GLU | C | 155 | 14760 | 15979 | 15203 | 91 | −2868 | 1293 | C |
| ATOM | 1117 | CG | GLU | C | 155 | 91.964 | 4.030 | 1.279 | 1.00 | 133.87 | | C |
| ANISOU | 1117 | CG | GLU | C | 155 | 15985 | 17941 | 16940 | −113 | −2719 | 1211 | C |
| ATOM | 1118 | CD | GLU | C | 155 | 91.067 | 5.250 | 1.394 | 1.00 | 151.31 | | C |
| ANISOU | 1118 | CD | GLU | C | 155 | 18208 | 20113 | 19170 | −522 | −2474 | 1292 | C |
| ATOM | 1119 | OE1 | GLU | C | 155 | 90.176 | 5.414 | 0.529 | 1.00 | 146.72 | | O |
| ANISOU | 1119 | OE1 | GLU | C | 155 | 17716 | 19419 | 18614 | −639 | −2253 | 1347 | O |
| ATOM | 1120 | OE2 | GLU | C | 155 | 91.291 | 6.069 | 2.316 | 1.00 | 139.50 | | O |
| ANISOU | 1120 | OE2 | GLU | C | 155 | 16619 | 18714 | 17669 | −710 | −2511 | 1280 | O |
| ATOM | 1121 | N | HIS | C | 156 | 91.374 | 3.533 | 4.436 | 1.00 | 126.14 | | N |
| ANISOU | 1121 | N | HIS | C | 156 | 15601 | 16676 | 15652 | −236 | −3199 | 1409 | N |
| ATOM | 1122 | CA | HIS | C | 156 | 92.153 | 3.910 | 5.623 | 1.00 | 129.08 | | C |
| ANISOU | 1122 | CA | HIS | C | 156 | 15845 | 17228 | 15972 | −230 | −3397 | 1357 | C |
| ATOM | 1123 | C | HIS | C | 156 | 91.270 | 3.940 | 6.895 | 1.00 | 133.04 | | C |
| ANISOU | 1123 | C | HIS | C | 156 | 16687 | 17573 | 16291 | −446 | −3481 | 1501 | C |
| ATOM | 1124 | O | HIS | C | 156 | 91.081 | 4.983 | 7.535 | 1.00 | 131.38 | | O |
| ANISOU | 1124 | O | HIS | C | 156 | 16371 | 17479 | 16068 | −712 | −3397 | 1479 | O |
| ATOM | 1125 | CB | HIS | C | 156 | 92.866 | 5.253 | 5.349 | 1.00 | 130.08 | | C |
| ANISOU | 1125 | CB | HIS | C | 156 | 15501 | 17672 | 16250 | −393 | −3230 | 1212 | C |
| ATOM | 1126 | CG | HIS | C | 156 | 93.958 | 5.614 | 6.308 | 1.00 | 136.39 | | C |
| ANISOU | 1126 | CG | HIS | C | 156 | 16059 | 18725 | 17038 | −336 | −3432 | 1097 | C |
| ATOM | 1127 | CD2 | HIS | C | 156 | 95.296 | 5.664 | 6.111 | 1.00 | 140.95 | | C |
| ANISOU | 1127 | CD2 | HIS | C | 156 | 16246 | 19604 | 17705 | −145 | −3521 | 927 | C |
| ATOM | 1128 | ND1 | HIS | C | 156 | 93.678 | 6.054 | 7.592 | 1.00 | 138.44 | | N |
| ANISOU | 1128 | ND1 | HIS | C | 156 | 16446 | 18973 | 17181 | −511 | −3540 | 1138 | N |
| ATOM | 1129 | CE1 | HIS | C | 156 | 94.852 | 6.318 | 8.144 | 1.00 | 140.57 | | C |
| ANISOU | 1129 | CE1 | HIS | C | 156 | 16420 | 19515 | 17477 | −407 | −3708 | 995 | C |
| ATOM | 1130 | NE2 | HIS | C | 156 | 95.855 | 6.099 | 7.292 | 1.00 | 142.49 | | N |
| ANISOU | 1130 | NE2 | HIS | C | 156 | 16338 | 19958 | 17844 | −192 | −3703 | 864 | N |
| ATOM | 1131 | N | MET | C | 157 | 90.738 | 2.758 | 7.245 | 1.00 | 131.26 | | N |
| ANISOU | 1131 | N | MET | C | 157 | 16875 | 17082 | 15918 | −330 | −3653 | 1645 | N |
| ATOM | 1132 | CA | MET | C | 157 | 89.860 | 2.499 | 8.392 | 1.00 | 131.28 | | C |
| ANISOU | 1132 | CA | MET | C | 157 | 17249 | 16925 | 15704 | −514 | −3749 | 1813 | C |
| ATOM | 1133 | C | MET | C | 157 | 90.130 | 1.052 | 8.899 | 1.00 | 137.21 | | C |
| ANISOU | 1133 | C | MET | C | 157 | 18346 | 17471 | 16316 | −231 | −4085 | 1927 | C |
| ATOM | 1134 | O | MET | C | 157 | 90.483 | 0.203 | 8.071 | 1.00 | 137.90 | | O |
| ANISOU | 1134 | O | MET | C | 157 | 18477 | 17430 | 16487 | 47 | −4158 | 1898 | O |
| ATOM | 1135 | CB | MET | C | 157 | 88.386 | 2.641 | 7.949 | 1.00 | 130.83 | | C |
| ANISOU | 1135 | CB | MET | C | 157 | 17413 | 16682 | 15615 | −803 | −3493 | 1921 | C |
| ATOM | 1136 | CG | MET | C | 157 | 87.502 | 3.385 | 8.935 | 1.00 | 133.51 | | C |
| ANISOU | 1136 | CG | MET | C | 157 | 17851 | 17070 | 15807 | −1146 | −3409 | 1983 | C |
| ATOM | 1137 | SD | MET | C | 157 | 86.020 | 2.440 | 9.387 | 1.00 | 137.61 | | S |
| ANISOU | 1137 | SD | MET | C | 157 | 18895 | 17309 | 16081 | −1333 | −3427 | 2215 | S |
| ATOM | 1138 | CE | MET | C | 157 | 84.901 | 3.758 | 9.787 | 1.00 | 131.11 | | C |
| ANISOU | 1138 | CE | MET | C | 157 | 17982 | 16596 | 15239 | −1730 | −3105 | 2172 | C |
| ATOM | 1139 | N | PRO | C | 158 | 89.979 | 0.732 | 10.217 | 1.00 | 134.18 | | N |
| ANISOU | 1139 | N | PRO | C | 158 | 18222 | 17044 | 15714 | −283 | −4301 | 2055 | N |
| ATOM | 1140 | CA | PRO | C | 158 | 90.245 | −0.653 | 10.676 | 1.00 | 136.72 | | C |
| ANISOU | 1140 | CA | PRO | C | 158 | 18911 | 17134 | 15901 | −14 | −4638 | 2188 | C |
| ATOM | 1141 | C | PRO | C | 158 | 89.366 | −1.718 | 10.003 | 1.00 | 138.33 | | C |
| ANISOU | 1141 | C | PRO | C | 158 | 19504 | 16966 | 16090 | −13 | −4614 | 2334 | C |
| ATOM | 1142 | O | PRO | C | 158 | 88.162 | −1.493 | 9.894 | 1.00 | 135.39 | | O |
| ANISOU | 1142 | O | PRO | C | 158 | 19286 | 16497 | 15659 | −346 | −4391 | 2436 | O |
| ATOM | 1143 | CB | PRO | C | 158 | 90.020 | −0.587 | 12.190 | 1.00 | 140.01 | | C |
| ANISOU | 1143 | CB | PRO | C | 158 | 19537 | 17598 | 16063 | −176 | −4800 | 2325 | C |
| ATOM | 1144 | CG | PRO | C | 158 | 89.230 | 0.641 | 12.411 | 1.00 | 141.56 | | C |
| ANISOU | 1144 | CG | PRO | C | 158 | 19578 | 17967 | 16244 | −573 | −4507 | 2293 | C |
| ATOM | 1145 | CD | PRO | C | 158 | 89.601 | 1.605 | 11.346 | 1.00 | 134.92 | | C |
| ANISOU | 1145 | CD | PRO | C | 158 | 18293 | 17304 | 15668 | −574 | −4263 | 2080 | C |
| ATOM | 1146 | N | PRO | C | 159 | 89.945 | −2.866 | 9.540 | 1.00 | 136.19 | | N |
| ANISOU | 1146 | N | PRO | C | 159 | 19382 | 16491 | 15873 | 359 | −4845 | 2325 | N |
| ATOM | 1147 | CA | PRO | C | 159 | 89.145 | −3.887 | 8.824 | 1.00 | 135.21 | | C |
| ANISOU | 1147 | CA | PRO | C | 159 | 19619 | 15995 | 15759 | 364 | −4830 | 2437 | C |
| ATOM | 1148 | C | PRO | C | 159 | 87.833 | −4.322 | 9.471 | 1.00 | 136.69 | | C |
| ANISOU | 1148 | C | PRO | C | 159 | 20277 | 15924 | 15736 | 20 | −4818 | 2700 | C |
| ATOM | 1149 | O | PRO | C | 159 | 86.930 | −4.756 | 8.748 | 1.00 | 134.52 | | O |
| ANISOU | 1149 | O | PRO | C | 159 | 20205 | 15414 | 15491 | −109 | −4685 | 2763 | O |
| ATOM | 1150 | CB | PRO | C | 159 | 90.107 | −5.065 | 8.695 | 1.00 | 140.90 | | C |
| ANISOU | 1150 | CB | PRO | C | 159 | 20473 | 16542 | 16519 | 847 | −5186 | 2392 | C |
| ATOM | 1151 | CG | PRO | C | 159 | 91.442 | −4.444 | 8.668 | 1.00 | 146.58 | | C |
| ANISOU | 1151 | CG | PRO | C | 159 | 20715 | 17630 | 17346 | 1113 | −5246 | 2162 | C |
| ATOM | 1152 | CD | PRO | C | 159 | 91.371 | −3.255 | 9.586 | 1.00 | 140.90 | | C |
| ANISOU | 1152 | CD | PRO | C | 159 | 19798 | 17201 | 16535 | 808 | −5131 | 2179 | C |
| ATOM | 1153 | N | GLU | C | 160 | 87.723 | −4.206 | 10.812 | 1.00 | 133.35 | | N |
| ANISOU | 1153 | N | GLU | C | 160 | 20010 | 15567 | 15091 | −137 | −4951 | 2844 | N |
| ATOM | 1154 | CA | GLU | C | 160 | 86.494 | −4.546 | 11.524 | 1.00 | 132.57 | | C |
| ANISOU | 1154 | CA | GLU | C | 160 | 20321 | 15295 | 14755 | −500 | −4926 | 3097 | C |
| ATOM | 1155 | C | GLU | C | 160 | 85.451 | −3.441 | 11.413 | 1.00 | 130.27 | | C |
| ANISOU | 1155 | C | GLU | C | 160 | 19850 | 15198 | 14448 | −919 | −4546 | 3063 | C |
| ATOM | 1156 | O | GLU | C | 160 | 84.306 | −3.737 | 11.068 | 1.00 | 128.82 | | O |
| ANISOU | 1156 | O | GLU | C | 160 | 19889 | 14840 | 14216 | −1171 | −4390 | 3168 | O |
| ATOM | 1157 | CB | GLU | C | 160 | 86.748 | −4.964 | 12.979 | 1.00 | 137.34 | | C |
| ANISOU | 1157 | CB | GLU | C | 160 | 21187 | 15901 | 15095 | −498 | −5225 | 3278 | C |
| ATOM | 1158 | CG | GLU | C | 160 | 86.671 | −6.472 | 13.189 | 1.00 | 151.81 | | C |
| ANISOU | 1158 | CG | GLU | C | 160 | 23555 | 17314 | 16810 | −352 | −5552 | 3505 | C |
| ATOM | 1159 | CD | GLU | C | 160 | 85.435 | −7.152 | 12.626 | 1.00 | 167.65 | | C |
| ANISOU | 1159 | CD | GLU | C | 160 | 25930 | 18987 | 18781 | −623 | −5431 | 3673 | C |
| ATOM | 1160 | OE1 | GLU | C | 160 | 85.565 | −7.858 | 11.600 | 1.00 | 154.16 | | O |
| ANISOU | 1160 | OE1 | GLU | C | 160 | 24305 | 17014 | 17257 | −414 | −5469 | 3608 | O |
| ATOM | 1161 | OE2 | GLU | C | 160 | 84.331 | −6.935 | 13.176 | 1.00 | 161.02 | | O |
| ANISOU | 1161 | OE2 | GLU | C | 160 | 25267 | 18183 | 17730 | −1051 | −5287 | 3845 | O |
| ATOM | 1162 | N | LYS | C | 161 | 85.854 | −2.168 | 11.646 | 1.00 | 122.69 | | N |
| ANISOU | 1162 | N | LYS | C | 161 | 18480 | 14593 | 13543 | −982 | −4403 | 2897 | N |
| ATOM | 1163 | CA | LYS | C | 161 | 84.965 | −1.006 | 11.516 | 1.00 | 117.83 | | C |
| ANISOU | 1163 | CA | LYS | C | 161 | 17663 | 14172 | 12937 | −1331 | −4063 | 2826 | C |
| ATOM | 1164 | C | LYS | C | 161 | 84.589 | −0.719 | 10.041 | 1.00 | 115.83 | | C |
| ANISOU | 1164 | C | LYS | C | 161 | 17226 | 13865 | 12917 | −1334 | −3789 | 2701 | C |
| ATOM | 1165 | O | LYS | C | 161 | 83.552 | −0.103 | 9.783 | 1.00 | 112.56 | | O |
| ANISOU | 1165 | O | LYS | C | 161 | 16770 | 13507 | 12492 | −1621 | −3521 | 2687 | O |
| ATOM | 1166 | CB | LYS | C | 161 | 85.537 | 0.224 | 12.237 | 1.00 | 119.65 | | C |
| ANISOU | 1166 | CB | LYS | C | 161 | 17548 | 14753 | 13161 | −1386 | −4034 | 2685 | C |
| ATOM | 1167 | CG | LYS | C | 161 | 85.165 | 0.261 | 13.720 | 1.00 | 131.87 | | C |
| ANISOU | 1167 | CG | LYS | C | 161 | 19298 | 16402 | 14406 | −1579 | −4163 | 2820 | C |
| ATOM | 1168 | CD | LYS | C | 161 | 86.341 | 0.631 | 14.626 | 1.00 | 138.74 | | C |
| ANISOU | 1168 | CD | LYS | C | 161 | 19966 | 17509 | 15241 | −1401 | −4381 | 2727 | C |
| ATOM | 1169 | CE | LYS | C | 161 | 86.914 | −0.548 | 15.384 | 1.00 | 149.59 | | C |
| ANISOU | 1169 | CE | LYS | C | 161 | 21653 | 18747 | 16438 | −1176 | −4751 | 2896 | C |
| ATOM | 1170 | NZ | LYS | C | 161 | 87.567 | −1.550 | 14.497 | 1.00 | 158.07 | | N |
| ANISOU | 1170 | NZ | LYS | C | 161 | 22812 | 19570 | 17678 | −817 | −4914 | 2898 | N |
| ATOM | 1171 | N | GLN | C | 162 | 85.419 | −1.203 | 9.083 | 1.00 | 110.75 | | N |
| ANISOU | 1171 | N | GLN | C | 162 | 16481 | 13129 | 12469 | −998 | −3867 | 2606 | N |
| ATOM | 1172 | CA | GLN | C | 162 | 85.181 | −1.135 | 7.639 | 1.00 | 107.22 | | C |
| ANISOU | 1172 | CA | GLN | C | 162 | 15891 | 12623 | 12224 | −943 | −3651 | 2498 | C |
| ATOM | 1173 | C | GLN | C | 162 | 83.987 | −2.046 | 7.368 | 1.00 | 111.24 | | C |
| ANISOU | 1173 | C | GLN | C | 162 | 16798 | 12828 | 12639 | −1101 | −3616 | 2652 | C |
| ATOM | 1174 | O | GLN | C | 162 | 83.001 | −1.614 | 6.770 | 1.00 | 108.87 | | O |
| ANISOU | 1174 | O | GLN | C | 162 | 16461 | 12539 | 12367 | −1332 | −3347 | 2632 | O |
| ATOM | 1175 | CB | GLN | C | 162 | 86.383 | −1.698 | 6.861 | 1.00 | 109.60 | | C |
| ANISOU | 1175 | CB | GLN | C | 162 | 16048 | 12899 | 12697 | −515 | −3810 | 2372 | C |
| ATOM | 1176 | CG | GLN | C | 162 | 87.549 | −0.753 | 6.662 | 1.00 | 112.85 | | C |
| ANISOU | 1176 | CG | GLN | C | 162 | 15978 | 13641 | 13258 | −362 | −3772 | 2174 | C |
| ATOM | 1177 | CD | GLN | C | 162 | 88.703 | −1.421 | 5.946 | 1.00 | 129.26 | | C |
| ANISOU | 1177 | CD | GLN | C | 162 | 17916 | 15726 | 15473 | 69 | −3937 | 2040 | C |
| ATOM | 1178 | NE2 | GLN | C | 162 | 89.686 | −0.635 | 5.544 | 1.00 | 121.62 | | N |
| ANISOU | 1178 | NE2 | GLN | C | 162 | 16495 | 15069 | 14645 | 188 | −3868 | 1855 | N |
| ATOM | 1179 | OE1 | GLN | C | 162 | 88.733 | −2.638 | 5.741 | 1.00 | 125.90 | | O |
| ANISOU | 1179 | OE1 | GLN | C | 162 | 17777 | 15034 | 15027 | 298 | −4132 | 2091 | O |
| ATOM | 1180 | N | LYS | C | 163 | 84.083 | −3.309 | 7.852 | 1.00 | 110.34 | | N |
| ANISOU | 1180 | N | LYS | C | 163 | 17074 | 12440 | 12409 | −981 | −3904 | 2807 | N |
| ATOM | 1181 | CA | LYS | C | 163 | 83.094 | −4.383 | 7.735 | 1.00 | 110.32 | | C |
| ANISOU | 1181 | CA | LYS | C | 163 | 17511 | 12100 | 12306 | −1122 | −3947 | 2979 | C |
| ATOM | 1182 | C | LYS | C | 163 | 81.765 | −4.026 | 8.422 | 1.00 | 111.25 | | C |
| ANISOU | 1182 | C | LYS | C | 163 | 17785 | 12273 | 12211 | −1590 | −3772 | 3121 | C |
| ATOM | 1183 | O | LYS | C | 163 | 80.707 | −4.411 | 7.921 | 1.00 | 109.37 | | O |
| ANISOU | 1183 | O | LYS | C | 163 | 17733 | 11875 | 11947 | −1798 | −3638 | 3181 | O |
| ATOM | 1184 | CB | LYS | C | 163 | 83.678 | −5.675 | 8.326 | 1.00 | 116.68 | | C |
| ANISOU | 1184 | CB | LYS | C | 163 | 18693 | 12617 | 13023 | −884 | −4341 | 3124 | C |
| ATOM | 1185 | CG | LYS | C | 163 | 83.040 | −6.952 | 7.798 | 1.00 | 130.00 | | C |
| ANISOU | 1185 | CG | LYS | C | 163 | 20800 | 13884 | 14708 | −887 | −4441 | 3240 | C |
| ATOM | 1186 | CD | LYS | C | 163 | 83.425 | −8.157 | 8.646 | 1.00 | 143.09 | | C |
| ANISOU | 1186 | CD | LYS | C | 163 | 22911 | 15230 | 16226 | −740 | −4845 | 3440 | C |
| ATOM | 1187 | CE | LYS | C | 163 | 82.559 | −8.296 | 9.876 | 1.00 | 153.33 | | C |
| ANISOU | 1187 | CE | LYS | C | 163 | 24548 | 16481 | 17229 | −1150 | −4873 | 3718 | C |
| ATOM | 1188 | NZ | LYS | C | 163 | 83.179 | −9.195 | 10.877 | 1.00 | 167.64 | | N |
| ANISOU | 1188 | NZ | LYS | C | 163 | 26695 | 18120 | 18881 | −979 | −5269 | 3904 | N |
| ATOM | 1189 | N | ARG | C | 164 | 81.832 | −3.281 | 9.555 | 1.00 | 107.07 | | N |
| ANISOU | 1189 | N | ARG | C | 164 | 17160 | 11996 | 11527 | −1748 | −3774 | 3153 | N |
| ATOM | 1190 | CA | ARG | C | 164 | 80.692 | −2.833 | 10.366 | 1.00 | 105.71 | | C |
| ANISOU | 1190 | CA | ARG | C | 164 | 17082 | 11959 | 11122 | −2166 | −3624 | 3256 | C |
| ATOM | 1191 | C | ARG | C | 164 | 79.715 | −1.891 | 9.633 | 1.00 | 104.38 | | C |
| ANISOU | 1191 | C | ARG | C | 164 | 16681 | 11950 | 11031 | −2401 | −3262 | 3122 | C |
| ATOM | 1192 | O | ARG | C | 164 | 78.500 | −2.098 | 9.727 | 1.00 | 103.40 | | O |
| ANISOU | 1192 | O | ARG | C | 164 | 16744 | 11786 | 10759 | −2709 | −3135 | 3215 | O |
| ATOM | 1193 | CB | ARG | C | 164 | 81.177 | −2.193 | 11.677 | 1.00 | 108.35 | | C |
| ANISOU | 1193 | CB | ARG | C | 164 | 17312 | 12561 | 11294 | −2208 | −3729 | 3267 | C |
| ATOM | 1194 | CG | ARG | C | 164 | 81.556 | −3.196 | 12.761 | 1.00 | 127.77 | | C |
| ANISOU | 1194 | CG | ARG | C | 164 | 20133 | 14876 | 13537 | −2139 | −4067 | 3486 | C |
| ATOM | 1195 | CD | ARG | C | 164 | 82.203 | −2.502 | 13.946 | 1.00 | 144.97 | | C |
| ANISOU | 1195 | CD | ARG | C | 164 | 22143 | 17351 | 15589 | −2106 | −4183 | 3450 | C |
| ATOM | 1196 | NE | ARG | C | 164 | 83.270 | −3.311 | 14.539 | 1.00 | 163.15 | | N |
| ANISOU | 1196 | NE | ARG | C | 164 | 24624 | 19529 | 17838 | −1798 | −4552 | 3546 | N |
| ATOM | 1197 | CZ | ARG | C | 164 | 83.988 | −2.948 | 15.598 | 1.00 | 182.96 | | C |
| ANISOU | 1197 | CZ | ARG | C | 164 | 27057 | 22253 | 20208 | −1722 | −4730 | 3545 | C |
| ATOM | 1198 | NH1 | ARG | C | 164 | 83.758 | −1.787 | 16.199 | 1.00 | 171.38 | | N |
| ANISOU | 1198 | NH1 | ARG | C | 164 | 25342 | 21130 | 18644 | −1939 | −4574 | 3445 | N |
| ATOM | 1199 | NH2 | ARG | C | 164 | 84.938 | −3.747 | 16.068 | 1.00 | 173.47 | | N |
| ANISOU | 1199 | NH2 | ARG | C | 164 | 26029 | 20924 | 18958 | −1412 | −5082 | 3630 | N |
| ATOM | 1200 | N | ILE | C | 165 | 80.239 | −0.847 | 8.933 | 1.00 | 97.28 | | N |
| ANISOU | 1200 | N | ILE | C | 165 | 15374 | 11241 | 10349 | −2266 | −3104 | 2908 | N |
| ATOM | 1201 | CA | ILE | C | 165 | 79.425 | 0.125 | 8.178 | 1.00 | 92.80 | | C |
| ANISOU | 1201 | CA | ILE | C | 165 | 14574 | 10814 | 9873 | −2440 | −2782 | 2772 | C |
| ATOM | 1202 | C | ILE | C | 165 | 79.056 | −0.411 | 6.781 | 1.00 | 93.82 | | C |
| ANISOU | 1202 | C | ILE | C | 165 | 14739 | 10746 | 10164 | −2349 | −2670 | 2733 | C |
| ATOM | 1203 | O | ILE | C | 165 | 78.096 | 0.074 | 6.188 | 1.00 | 91.05 | | O |
| ANISOU | 1203 | O | ILE | C | 165 | 14305 | 10454 | 9836 | −2523 | −2426 | 2668 | O |
| ATOM | 1204 | CB | ILE | C | 165 | 80.006 | 1.567 | 8.124 | 1.00 | 93.81 | | C |
| ANISOU | 1204 | CB | ILE | C | 165 | 14284 | 11223 | 10136 | −2401 | −2658 | 2583 | C |
| ATOM | 1205 | CG1 | ILE | C | 165 | 81.369 | 1.628 | 7.430 | 1.00 | 94.02 | | C |
| ANISOU | 1205 | CG1 | ILE | C | 165 | 14068 | 11259 | 10394 | −2064 | −2743 | 2474 | C |
| ATOM | 1206 | CG2 | ILE | C | 165 | 80.045 | 2.221 | 9.500 | 1.00 | 95.64 | | C |
| ANISOU | 1206 | CG2 | ILE | C | 165 | 14482 | 11674 | 10184 | −2560 | −2722 | 2592 | C |
| ATOM | 1207 | CD1 | ILE | C | 165 | 81.371 | 2.595 | 6.377 | 1.00 | 94.78 | | C |
| ANISOU | 1207 | CD1 | ILE | C | 165 | 13869 | 11447 | 10695 | −2046 | −2505 | 2325 | C |
| ATOM | 1208 | N | ALA | C | 166 | 79.801 | −1.414 | 6.272 | 1.00 | 90.94 | | N |
| ANISOU | 1208 | N | ALA | C | 166 | 14499 | 10155 | 9899 | −2063 | −2860 | 2761 | N |
| ATOM | 1209 | CA | ALA | C | 166 | 79.505 | −2.081 | 5.003 | 1.00 | 89.93 | | C |
| ANISOU | 1209 | CA | ALA | C | 166 | 14441 | 9824 | 9904 | −1951 | −2797 | 2719 | C |
| ATOM | 1210 | C | ALA | C | 166 | 78.219 | −2.915 | 5.195 | 1.00 | 94.63 | | C |
| ANISOU | 1210 | C | ALA | C | 166 | 15413 | 10212 | 10329 | −2233 | −2786 | 2872 | C |
| ATOM | 1211 | O | ALA | C | 166 | 77.394 | −3.007 | 4.282 | 1.00 | 92.78 | | O |
| ANISOU | 1211 | O | ALA | C | 166 | 15190 | 9912 | 10151 | −2322 | −2613 | 2821 | O |
| ATOM | 1212 | CB | ALA | C | 166 | 80.663 | −2.980 | 4.601 | 1.00 | 92.89 | | C |
| ANISOU | 1212 | CB | ALA | C | 166 | 14866 | 10025 | 10404 | −1554 | −3046 | 2693 | C |
| ATOM | 1213 | N | GLN | C | 167 | 78.046 | −3.482 | 6.416 | 1.00 | 93.33 | | N |
| ANISOU | 1213 | N | GLN | C | 167 | 15546 | 9971 | 9945 | −2393 | −2967 | 3062 | N |
| ATOM | 1214 | CA | GLN | C | 167 | 76.881 | −4.249 | 6.863 | 1.00 | 93.92 | | C |
| ANISOU | 1214 | CA | GLN | C | 167 | 15988 | 9887 | 9811 | −2723 | −2976 | 3243 | C |
| ATOM | 1215 | C | GLN | C | 167 | 75.674 | −3.299 | 6.913 | 1.00 | 93.99 | | C |
| ANISOU | 1215 | C | GLN | C | 167 | 15829 | 10167 | 9717 | −3074 | −2669 | 3180 | C |
| ATOM | 1216 | O | GLN | C | 167 | 74.640 | −3.598 | 6.318 | 1.00 | 93.35 | | O |
| ANISOU | 1216 | O | GLN | C | 167 | 15845 | 10012 | 9612 | −3269 | −2526 | 3180 | O |
| ATOM | 1217 | CB | GLN | C | 167 | 77.176 | −4.881 | 8.244 | 1.00 | 98.64 | | C |
| ANISOU | 1217 | CB | GLN | C | 167 | 16897 | 10399 | 10182 | −2790 | −3249 | 3465 | C |
| ATOM | 1218 | CG | GLN | C | 167 | 75.968 | −5.461 | 8.988 | 1.00 | 115.26 | | C |
| ANISOU | 1218 | CG | GLN | C | 167 | 19351 | 12433 | 12009 | −3215 | −3239 | 3681 | C |
| ATOM | 1219 | CD | GLN | C | 167 | 75.934 | −5.063 | 10.451 | 1.00 | 134.90 | | C |
| ANISOU | 1219 | CD | GLN | C | 167 | 21878 | 15154 | 14224 | −3412 | −3302 | 3808 | C |
| ATOM | 1220 | NE2 | GLN | C | 167 | 75.846 | −3.761 | 10.734 | 1.00 | 120.18 | | N |
| ANISOU | 1220 | NE2 | GLN | C | 167 | 19658 | 13669 | 12335 | −3494 | −3102 | 3655 | N |
| ATOM | 1221 | OE1 | GLN | C | 167 | 75.962 | −5.918 | 11.347 | 1.00 | 135.54 | | O |
| ANISOU | 1221 | OE1 | GLN | C | 167 | 22318 | 15075 | 14105 | −3493 | −3536 | 4043 | O |
| ATOM | 1222 | N | GLU | C | 168 | 75.843 | −2.136 | 7.571 | 1.00 | 87.81 | | N |
| ANISOU | 1222 | N | GLU | C | 168 | 14779 | 9700 | 8886 | −3127 | −2579 | 3101 | N |
| ATOM | 1223 | CA | GLU | C | 168 | 74.849 | −1.077 | 7.715 | 1.00 | 85.15 | | C |
| ANISOU | 1223 | CA | GLU | C | 168 | 14245 | 9652 | 8457 | −3400 | −2317 | 3003 | C |
| ATOM | 1224 | C | GLU | C | 168 | 74.342 | −0.555 | 6.348 | 1.00 | 86.12 | | C |
| ANISOU | 1224 | C | GLU | C | 168 | 14153 | 9799 | 8768 | −3365 | −2068 | 2829 | C |
| ATOM | 1225 | O | GLU | C | 168 | 73.133 | −0.530 | 6.126 | 1.00 | 85.70 | | O |
| ANISOU | 1225 | O | GLU | C | 168 | 14145 | 9800 | 8616 | −3614 | −1900 | 2815 | O |
| ATOM | 1226 | CB | GLU | C | 168 | 75.449 | 0.071 | 8.543 | 1.00 | 86.06 | | C |
| ANISOU | 1226 | CB | GLU | C | 168 | 14097 | 10052 | 8550 | −3363 | −2318 | 2912 | C |
| ATOM | 1227 | CG | GLU | C | 168 | 74.421 | 1.053 | 9.083 | 1.00 | 96.24 | | C |
| ANISOU | 1227 | CG | GLU | C | 168 | 15264 | 11639 | 9666 | −3661 | −2126 | 2837 | C |
| ATOM | 1228 | CD | GLU | C | 168 | 74.963 | 2.310 | 9.736 | 1.00 | 116.14 | | C |
| ANISOU | 1228 | CD | GLU | C | 168 | 17493 | 14429 | 12205 | −3615 | −2105 | 2694 | C |
| ATOM | 1229 | OE1 | GLU | C | 168 | 76.200 | 2.425 | 9.902 | 1.00 | 106.24 | | O |
| ANISOU | 1229 | OE1 | GLU | C | 168 | 16135 | 13154 | 11075 | −3378 | −2257 | 2673 | O |
| ATOM | 1230 | OE2 | GLU | C | 168 | 74.140 | 3.192 | 10.071 | 1.00 | 111.44 | | O |
| ANISOU | 1230 | OE2 | GLU | C | 168 | 16765 | 14073 | 11504 | −3812 | −1942 | 2584 | O |
| ATOM | 1231 | N | THR | C | 169 | 75.261 | −0.153 | 5.440 | 1.00 | 79.84 | | N |
| ANISOU | 1231 | N | THR | C | 169 | 13122 | 8987 | 8228 | −3062 | −2047 | 2699 | N |
| ATOM | 1232 | CA | THR | C | 169 | 74.942 | 0.379 | 4.109 | 1.00 | 76.41 | | C |
| ANISOU | 1232 | CA | THR | C | 169 | 12474 | 8586 | 7972 | −2992 | −1826 | 2545 | C |
| ATOM | 1233 | C | THR | C | 169 | 73.994 | −0.556 | 3.366 | 1.00 | 78.77 | | C |
| ANISOU | 1233 | C | THR | C | 169 | 12992 | 8696 | 8243 | −3090 | −1774 | 2583 | C |
| ATOM | 1234 | O | THR | C | 169 | 72.966 | −0.118 | 2.846 | 1.00 | 75.59 | | O |
| ANISOU | 1234 | O | THR | C | 169 | 12507 | 8397 | 7817 | −3252 | −1563 | 2500 | O |
| ATOM | 1235 | CB | THR | C | 169 | 76.240 | 0.641 | 3.321 | 1.00 | 84.87 | | C |
| ANISOU | 1235 | CB | THR | C | 169 | 13316 | 9643 | 9288 | −2644 | −1867 | 2448 | C |
| ATOM | 1236 | CG2 | THR | C | 169 | 75.994 | 1.341 | 1.997 | 1.00 | 81.06 | | C |
| ANISOU | 1236 | CG2 | THR | C | 169 | 12593 | 9234 | 8972 | −2575 | −1635 | 2302 | C |
| ATOM | 1237 | OG1 | THR | C | 169 | 77.121 | 1.435 | 4.111 | 1.00 | 85.52 | | O |
| ANISOU | 1237 | OG1 | THR | C | 169 | 13211 | 9899 | 9385 | −2586 | −1937 | 2417 | O |
| ATOM | 1238 | N | LEU | C | 170 | 74.339 | −1.850 | 3.359 | 1.00 | 77.92 | | N |
| ANISOU | 1238 | N | LEU | C | 170 | 13168 | 8306 | 8134 | −2989 | −1984 | 2699 | N |
| ATOM | 1239 | CA | LEU | C | 170 | 73.575 | −2.908 | 2.713 | 1.00 | 78.53 | | C |
| ANISOU | 1239 | CA | LEU | C | 170 | 13497 | 8144 | 8197 | −3071 | −1990 | 2741 | C |
| ATOM | 1240 | C | LEU | C | 170 | 72.216 | −3.112 | 3.354 | 1.00 | 83.33 | | C |
| ANISOU | 1240 | C | LEU | C | 170 | 14293 | 8802 | 8567 | −3488 | −1907 | 2838 | C |
| ATOM | 1241 | O | LEU | C | 170 | 71.232 | −3.323 | 2.643 | 1.00 | 83.08 | | O |
| ANISOU | 1241 | O | LEU | C | 170 | 14289 | 8749 | 8530 | −3634 | −1764 | 2782 | O |
| ATOM | 1242 | CB | LEU | C | 170 | 74.370 | −4.220 | 2.746 | 1.00 | 81.21 | | C |
| ANISOU | 1242 | CB | LEU | C | 170 | 14126 | 8145 | 8585 | −2855 | −2285 | 2848 | C |
| ATOM | 1243 | CG | LEU | C | 170 | 75.190 | −4.501 | 1.509 | 1.00 | 85.23 | | C |
| ANISOU | 1243 | CG | LEU | C | 170 | 14512 | 8538 | 9335 | −2469 | −2325 | 2708 | C |
| ATOM | 1244 | CD1 | LEU | C | 170 | 76.250 | −5.506 | 1.787 | 1.00 | 87.93 | | C |
| ANISOU | 1244 | CD1 | LEU | C | 170 | 15060 | 8628 | 9722 | −2188 | −2645 | 2780 | C |
| ATOM | 1245 | CD2 | LEU | C | 170 | 74.314 | −4.983 | 0.374 | 1.00 | 88.64 | | C |
| ANISOU | 1245 | CD2 | LEU | C | 170 | 15017 | 8838 | 9825 | −2518 | −2208 | 2631 | C |
| ATOM | 1246 | N | GLU | C | 171 | 72.170 | −3.043 | 4.690 | 1.00 | 80.41 | | N |
| ANISOU | 1246 | N | GLU | C | 171 | 14032 | 8530 | 7989 | −3681 | −1995 | 2973 | N |
| ATOM | 1247 | CA | GLU | C | 171 | 70.982 | −3.236 | 5.517 | 1.00 | 81.11 | | C |
| ANISOU | 1247 | CA | GLU | C | 171 | 14291 | 8723 | 7803 | −4099 | −1932 | 3083 | C |
| ATOM | 1248 | C | GLU | C | 171 | 69.966 | −2.105 | 5.337 | 1.00 | 82.50 | | C |
| ANISOU | 1248 | C | GLU | C | 171 | 14180 | 9244 | 7921 | −4287 | −1643 | 2914 | C |
| ATOM | 1249 | O | GLU | C | 171 | 68.792 | −2.357 | 5.057 | 1.00 | 81.31 | | O |
| ANISOU | 1249 | O | GLU | C | 171 | 14089 | 9137 | 7668 | −4543 | −1509 | 2897 | O |
| ATOM | 1250 | CB | GLU | C | 171 | 71.395 | −3.263 | 7.010 | 1.00 | 84.43 | | C |
| ANISOU | 1250 | CB | GLU | C | 171 | 14841 | 9227 | 8012 | −4201 | −2099 | 3249 | C |
| ATOM | 1251 | CG | GLU | C | 171 | 71.445 | −4.605 | 7.719 | 1.00 | 96.23 | | C |
| ANISOU | 1251 | CG | GLU | C | 171 | 16777 | 10433 | 9354 | −4311 | −2356 | 3512 | C |
| ATOM | 1252 | CD | GLU | C | 171 | 71.492 | −4.515 | 9.242 | 1.00 | 118.48 | | C |
| ANISOU | 1252 | CD | GLU | C | 171 | 19711 | 13418 | 11888 | −4506 | −2464 | 3680 | C |
| ATOM | 1253 | OE1 | GLU | C | 171 | 70.784 | −5.321 | 9.891 | 1.00 | 121.68 | | O |
| ANISOU | 1253 | OE1 | GLU | C | 171 | 20443 | 13737 | 12054 | −4826 | −2535 | 3893 | O |
| ATOM | 1254 | OE2 | GLU | C | 171 | 72.216 | −3.647 | 9.791 | 1.00 | 98.28 | | O |
| ANISOU | 1254 | OE2 | GLU | C | 171 | 16921 | 11086 | 9334 | −4358 | −2478 | 3601 | O |
| ATOM | 1255 | N | ILE | C | 172 | 70.442 | −0.859 | 5.511 | 1.00 | 78.27 | | N |
| ANISOU | 1255 | N | ILE | C | 172 | 13337 | 8950 | 7453 | −4153 | −1563 | 2781 | N |
| ATOM | 1256 | CA | ILE | C | 172 | 69.670 | 0.375 | 5.622 | 1.00 | 76.62 | | C |
| ANISOU | 1256 | CA | ILE | C | 172 | 12859 | 9077 | 7175 | −4291 | −1342 | 2618 | C |
| ATOM | 1257 | C | ILE | C | 172 | 69.715 | 1.360 | 4.442 | 1.00 | 76.56 | | C |
| ANISOU | 1257 | C | ILE | C | 172 | 12549 | 9145 | 7397 | −4090 | −1164 | 2409 | C |
| ATOM | 1258 | O | ILE | C | 172 | 68.673 | 1.851 | 4.004 | 1.00 | 74.35 | | O |
| ANISOU | 1258 | O | ILE | C | 172 | 12149 | 9027 | 7073 | −4217 | −975 | 2284 | O |
| ATOM | 1259 | CB | ILE | C | 172 | 70.165 | 1.124 | 6.912 | 1.00 | 80.94 | | C |
| ANISOU | 1259 | CB | ILE | C | 172 | 13312 | 9845 | 7597 | −4320 | −1415 | 2627 | C |
| ATOM | 1260 | CG1 | ILE | C | 172 | 70.580 | 0.159 | 8.091 | 1.00 | 84.63 | | C |
| ANISOU | 1260 | CG1 | ILE | C | 172 | 14081 | 10207 | 7866 | −4419 | −1651 | 2862 | C |
| ATOM | 1261 | CG2 | ILE | C | 172 | 69.177 | 2.194 | 7.380 | 1.00 | 81.65 | | C |
| ANISOU | 1261 | CG2 | ILE | C | 172 | 13203 | 10291 | 7529 | −4527 | −1230 | 2477 | C |
| ATOM | 1262 | CD1 | ILE | C | 172 | 69.484 | −0.687 | 8.859 | 1.00 | 95.59 | | C |
| ANISOU | 1262 | CD1 | ILE | C | 172 | 15754 | 11627 | 8938 | −4812 | −1657 | 3033 | C |
| ATOM | 1263 | N | TYR | C | 173 | 70.915 | 1.733 | 4.024 | 1.00 | 72.21 | | N |
| ANISOU | 1263 | N | TYR | C | 173 | 11858 | 8512 | 7067 | −3790 | −1228 | 2370 | N |
| ATOM | 1264 | CA | TYR | C | 173 | 71.105 | 2.810 | 3.084 | 1.00 | 70.13 | | C |
| ANISOU | 1264 | CA | TYR | C | 173 | 11302 | 8343 | 7000 | −3619 | −1073 | 2202 | C |
| ATOM | 1265 | C | TYR | C | 173 | 70.761 | 2.465 | 1.643 | 1.00 | 74.10 | | C |
| ANISOU | 1265 | C | TYR | C | 173 | 11789 | 8725 | 7641 | −3510 | −963 | 2140 | C |
| ATOM | 1266 | O | TYR | C | 173 | 69.895 | 3.153 | 1.102 | 1.00 | 72.01 | | O |
| ANISOU | 1266 | O | TYR | C | 173 | 11384 | 8600 | 7377 | −3572 | −779 | 2017 | O |
| ATOM | 1267 | CB | TYR | C | 173 | 72.509 | 3.395 | 3.234 | 1.00 | 71.48 | | C |
| ANISOU | 1267 | CB | TYR | C | 173 | 11306 | 8522 | 7331 | −3387 | −1171 | 2183 | C |
| ATOM | 1268 | CG | TYR | C | 173 | 72.657 | 4.050 | 4.586 | 1.00 | 74.32 | | C |
| ANISOU | 1268 | CG | TYR | C | 173 | 11619 | 9065 | 7554 | −3508 | −1236 | 2185 | C |
| ATOM | 1269 | CD1 | TYR | C | 173 | 72.369 | 5.398 | 4.761 | 1.00 | 75.04 | | C |
| ANISOU | 1269 | CD1 | TYR | C | 173 | 11480 | 9369 | 7661 | −3558 | −1112 | 2036 | C |
| ATOM | 1270 | CD2 | TYR | C | 173 | 72.967 | 3.298 | 5.715 | 1.00 | 78.07 | | C |
| ANISOU | 1270 | CD2 | TYR | C | 173 | 12301 | 9501 | 7860 | −3583 | −1428 | 2332 | C |
| ATOM | 1271 | CE1 | TYR | C | 173 | 72.458 | 6.000 | 6.014 | 1.00 | 78.30 | | C |
| ANISOU | 1271 | CE1 | TYR | C | 173 | 11847 | 9963 | 7941 | −3664 | −1179 | 2010 | C |
| ATOM | 1272 | CE2 | TYR | C | 173 | 73.018 | 3.878 | 6.980 | 1.00 | 80.60 | | C |
| ANISOU | 1272 | CE2 | TYR | C | 173 | 12580 | 10021 | 8023 | −3703 | −1485 | 2325 | C |
| ATOM | 1273 | CZ | TYR | C | 173 | 72.773 | 5.234 | 7.127 | 1.00 | 90.04 | | C |
| ANISOU | 1273 | CZ | TYR | C | 173 | 13523 | 11440 | 9248 | −3739 | −1359 | 2151 | C |
| ATOM | 1274 | OH | TYR | C | 173 | 72.852 | 5.815 | 8.377 | 1.00 | 93.75 | | O |
| ANISOU | 1274 | OH | TYR | C | 173 | 13942 | 12115 | 9562 | −3840 | −1427 | 2117 | O |
| ATOM | 1275 | N | ALA | C | 174 | 71.389 | 1.434 | 1.014 | 1.00 | 72.21 | | N |
| ANISOU | 1275 | N | ALA | C | 174 | 11686 | 8242 | 7509 | −3333 | −1082 | 2207 | N |
| ATOM | 1276 | CA | ALA | C | 174 | 71.047 | 1.069 | −0.377 | 1.00 | 70.90 | | C |
| ANISOU | 1276 | CA | ALA | C | 174 | 11502 | 7977 | 7460 | −3221 | −983 | 2131 | C |
| ATOM | 1277 | C | ALA | C | 174 | 69.525 | 0.783 | −0.521 | 1.00 | 74.18 | | C |
| ANISOU | 1277 | C | ALA | C | 174 | 12024 | 8439 | 7724 | −3490 | −857 | 2101 | C |
| ATOM | 1278 | O | ALA | C | 174 | 68.931 | 1.409 | −1.398 | 1.00 | 71.74 | | O |
| ANISOU | 1278 | O | ALA | C | 174 | 11545 | 8246 | 7468 | −3463 | −679 | 1970 | O |
| ATOM | 1279 | CB | ALA | C | 174 | 71.902 | −0.084 | −0.883 | 1.00 | 73.15 | | C |
| ANISOU | 1279 | CB | ALA | C | 174 | 11937 | 7997 | 7858 | −2991 | −1157 | 2188 | C |
| ATOM | 1280 | N | PRO | C | 175 | 68.833 | 0.046 | 0.414 | 1.00 | 72.54 | | N |
| ANISOU | 1280 | N | PRO | C | 175 | 12056 | 8199 | 7307 | −3776 | −929 | 2213 | N |
| ATOM | 1281 | CA | PRO | C | 175 | 67.362 | −0.064 | 0.327 | 1.00 | 72.33 | | C |
| ANISOU | 1281 | CA | PRO | C | 175 | 12067 | 8293 | 7123 | −4062 | −785 | 2160 | C |
| ATOM | 1282 | C | PRO | C | 175 | 66.648 | 1.296 | 0.293 | 1.00 | 75.76 | | C |
| ANISOU | 1282 | C | PRO | C | 175 | 12222 | 9053 | 7509 | −4123 | −582 | 1996 | C |
| ATOM | 1283 | O | PRO | C | 175 | 65.637 | 1.432 | −0.386 | 1.00 | 75.12 | | O |
| ANISOU | 1283 | O | PRO | C | 175 | 12068 | 9077 | 7396 | −4203 | −434 | 1878 | O |
| ATOM | 1284 | CB | PRO | C | 175 | 66.997 | −0.818 | 1.606 | 1.00 | 76.27 | | C |
| ANISOU | 1284 | CB | PRO | C | 175 | 12828 | 8764 | 7387 | −4368 | −904 | 2330 | C |
| ATOM | 1285 | CG | PRO | C | 175 | 68.182 | −1.610 | 1.915 | 1.00 | 82.08 | | C |
| ANISOU | 1285 | CG | PRO | C | 175 | 13762 | 9228 | 8199 | −4198 | −1141 | 2485 | C |
| ATOM | 1286 | CD | PRO | C | 175 | 69.334 | −0.738 | 1.564 | 1.00 | 76.16 | | C |
| ANISOU | 1286 | CD | PRO | C | 175 | 12762 | 8527 | 7649 | −3863 | −1144 | 2398 | C |
| ATOM | 1287 | N | LEU | C | 176 | 67.181 | 2.306 | 1.004 | 1.00 | 72.20 | | N |
| ANISOU | 1287 | N | LEU | C | 176 | 11618 | 8756 | 7060 | −4070 | −589 | 1973 | N |
| ATOM | 1288 | CA | LEU | C | 176 | 66.613 | 3.648 | 1.002 | 1.00 | 70.15 | | C |
| ANISOU | 1288 | CA | LEU | C | 176 | 11109 | 8767 | 6776 | −4088 | −432 | 1806 | C |
| ATOM | 1289 | C | LEU | C | 176 | 66.895 | 4.368 | −0.291 | 1.00 | 72.86 | | C |
| ANISOU | 1289 | C | LEU | C | 176 | 11258 | 9087 | 7339 | −3834 | −325 | 1689 | C |
| ATOM | 1290 | O | LEU | C | 176 | 66.012 | 5.049 | −0.783 | 1.00 | 71.56 | | O |
| ANISOU | 1290 | O | LEU | C | 176 | 10965 | 9077 | 7149 | −3859 | −178 | 1548 | O |
| ATOM | 1291 | CB | LEU | C | 176 | 67.117 | 4.464 | 2.175 | 1.00 | 70.29 | | C |
| ANISOU | 1291 | CB | LEU | C | 176 | 11044 | 8929 | 6736 | −4109 | −496 | 1809 | C |
| ATOM | 1292 | CG | LEU | C | 176 | 66.134 | 4.547 | 3.306 | 1.00 | 76.46 | | C |
| ANISOU | 1292 | CG | LEU | C | 176 | 11856 | 9958 | 7237 | −4402 | −467 | 1787 | C |
| ATOM | 1293 | CD1 | LEU | C | 176 | 66.850 | 4.643 | 4.633 | 1.00 | 78.20 | | C |
| ANISOU | 1293 | CD1 | LEU | C | 176 | 12122 | 10232 | 7358 | −4447 | −611 | 1877 | C |
| ATOM | 1294 | CD2 | LEU | C | 176 | 65.144 | 5.700 | 3.093 | 1.00 | 77.72 | | C |
| ANISOU | 1294 | CD2 | LEU | C | 176 | 11790 | 10386 | 7353 | −4430 | −295 | 1564 | C |
| ATOM | 1295 | N | ALA | C | 177 | 68.108 | 4.208 | −0.854 | 1.00 | 69.67 | | N |
| ANISOU | 1295 | N | ALA | C | 177 | 10831 | 8506 | 7134 | −3587 | −401 | 1745 | N |
| ATOM | 1296 | CA | ALA | C | 177 | 68.514 | 4.811 | −2.131 | 1.00 | 67.93 | | C |
| ANISOU | 1296 | CA | ALA | C | 177 | 10432 | 8265 | 7113 | −3349 | −304 | 1665 | C |
| ATOM | 1297 | C | ALA | C | 177 | 67.683 | 4.211 | −3.261 | 1.00 | 71.44 | | C |
| ANISOU | 1297 | C | ALA | C | 177 | 10923 | 8666 | 7554 | −3337 | −209 | 1608 | C |
| ATOM | 1298 | O | ALA | C | 177 | 67.392 | 4.889 | −4.246 | 1.00 | 68.55 | | O |
| ANISOU | 1298 | O | ALA | C | 177 | 10405 | 8380 | 7261 | −3229 | −75 | 1505 | O |
| ATOM | 1299 | CB | ALA | C | 177 | 69.990 | 4.557 | −2.379 | 1.00 | 69.16 | | C |
| ANISOU | 1299 | CB | ALA | C | 177 | 10563 | 8273 | 7441 | −3119 | −419 | 1743 | C |
| ATOM | 1300 | N | HIS | C | 178 | 67.277 | 2.937 | −3.086 | 1.00 | 70.84 | | N |
| ANISOU | 1300 | N | HIS | C | 178 | 11072 | 8459 | 7386 | −3461 | −288 | 1677 | N |
| ATOM | 1301 | CA | HIS | C | 178 | 66.413 | 2.192 | −3.990 | 1.00 | 71.40 | | C |
| ANISOU | 1301 | CA | HIS | C | 178 | 11224 | 8475 | 7431 | −3501 | −227 | 1620 | C |
| ATOM | 1302 | C | HIS | C | 178 | 65.022 | 2.814 | −3.866 | 1.00 | 73.26 | | C |
| ANISOU | 1302 | C | HIS | C | 178 | 11368 | 8955 | 7510 | −3706 | −75 | 1498 | C |
| ATOM | 1303 | O | HIS | C | 178 | 64.512 | 3.343 | −4.861 | 1.00 | 69.80 | | O |
| ANISOU | 1303 | O | HIS | C | 178 | 10784 | 8616 | 7118 | −3603 | 56 | 1369 | O |
| ATOM | 1304 | CB | HIS | C | 178 | 66.425 | 0.710 | −3.593 | 1.00 | 75.32 | | C |
| ANISOU | 1304 | CB | HIS | C | 178 | 12012 | 8743 | 7862 | −3623 | −385 | 1742 | C |
| ATOM | 1305 | CG | HIS | C | 178 | 65.364 | −0.124 | −4.232 | 1.00 | 80.47 | | C |
| ANISOU | 1305 | CG | HIS | C | 178 | 12783 | 9341 | 8450 | −3762 | −340 | 1687 | C |
| ATOM | 1306 | CD2 | HIS | C | 178 | 64.266 | −0.695 | −3.679 | 1.00 | 84.38 | | C |
| ANISOU | 1306 | CD2 | HIS | C | 178 | 13429 | 9873 | 8758 | −4094 | −333 | 1711 | C |
| ATOM | 1307 | ND1 | HIS | C | 178 | 65.430 | −0.475 | −5.571 | 1.00 | 82.42 | | N |
| ANISOU | 1307 | ND1 | HIS | C | 178 | 12994 | 9494 | 8828 | −3558 | −305 | 1595 | N |
| ATOM | 1308 | CE1 | HIS | C | 178 | 64.356 | −1.222 | −5.797 | 1.00 | 83.21 | | C |
| ANISOU | 1308 | CE1 | HIS | C | 178 | 13222 | 9565 | 8828 | −3766 | −282 | 1553 | C |
| ATOM | 1309 | NE2 | HIS | C | 178 | 63.625 | −1.379 | −4.689 | 1.00 | 84.69 | | N |
| ANISOU | 1309 | NE2 | HIS | C | 178 | 13522 | 9832 | 8826 | −4103 | −294 | 1624 | N |
| ATOM | 1310 | N | ARG | C | 179 | 64.472 | 2.854 | −2.606 | 1.00 | 71.84 | | N |
| ANISOU | 1310 | N | ARG | C | 179 | 11254 | 8906 | 7137 | −3976 | −98 | 1532 | N |
| ATOM | 1311 | CA | ARG | C | 179 | 63.176 | 3.457 | −2.220 | 1.00 | 71.95 | | C |
| ANISOU | 1311 | CA | ARG | C | 179 | 11166 | 9210 | 6963 | −4189 | 29 | 1402 | C |
| ATOM | 1312 | C | ARG | C | 179 | 63.096 | 4.816 | −2.893 | 1.00 | 74.08 | | C |
| ANISOU | 1312 | C | ARG | C | 179 | 11185 | 9630 | 7331 | −3987 | 150 | 1243 | C |
| ATOM | 1313 | O | ARG | C | 179 | 62.124 | 5.099 | −3.593 | 1.00 | 73.74 | | O |
| ANISOU | 1313 | O | ARG | C | 179 | 11044 | 9724 | 7249 | −3985 | 269 | 1099 | O |
| ATOM | 1314 | CB | ARG | C | 179 | 63.085 | 3.629 | −0.684 | 1.00 | 73.74 | | C |
| ANISOU | 1314 | CB | ARG | C | 179 | 11437 | 9583 | 6999 | −4417 | −29 | 1464 | C |
| ATOM | 1315 | CG | ARG | C | 179 | 61.714 | 3.329 | −0.053 | 1.00 | 82.81 | | C |
| ANISOU | 1315 | CG | ARG | C | 179 | 12622 | 10965 | 7876 | −4763 | 40 | 1416 | C |
| ATOM | 1316 | CD | ARG | C | 179 | 61.578 | 1.901 | 0.480 | 1.00 | 90.83 | | C |
| ANISOU | 1316 | CD | ARG | C | 179 | 13925 | 11824 | 8764 | −5027 | −68 | 1605 | C |
| ATOM | 1317 | NE | ARG | C | 179 | 62.789 | 1.410 | 1.154 | 1.00 | 96.40 | | N |
| ANISOU | 1317 | NE | ARG | C | 179 | 14804 | 12297 | 9524 | −4957 | −251 | 1806 | N |
| ATOM | 1318 | CZ | ARG | C | 179 | 63.091 | 1.621 | 2.432 | 1.00 | 105.52 | | C |
| ANISOU | 1318 | CZ | ARG | C | 179 | 15986 | 13567 | 10538 | −5067 | −322 | 1889 | C |
| ATOM | 1319 | NH1 | ARG | C | 179 | 64.218 | 1.142 | 2.941 | 1.00 | 85.47 | | N |
| ANISOU | 1319 | NH1 | ARG | C | 179 | 13605 | 10811 | 8059 | −4970 | −502 | 2063 | N |
| ATOM | 1320 | NH2 | ARG | C | 179 | 62.272 | 2.319 | 3.210 | 1.00 | 93.74 | | N |
| ANISOU | 1320 | NH2 | ARG | C | 179 | 14353 | 12427 | 8837 | −5256 | −223 | 1782 | N |
| ATOM | 1321 | N | LEU | C | 180 | 64.187 | 5.604 | −2.745 | 1.00 | 68.71 | | N |
| ANISOU | 1321 | N | LEU | C | 180 | 10416 | 8900 | 6792 | −3805 | 105 | 1279 | N |
| ATOM | 1322 | CA | LEU | C | 180 | 64.444 | 6.896 | −3.360 | 1.00 | 51.94 | | C |
| ANISOU | 1322 | CA | LEU | C | 180 | 8093 | 6839 | 4802 | −3600 | 183 | 1180 | C |
| ATOM | 1323 | C | LEU | C | 180 | 64.941 | 6.611 | −4.736 | 1.00 | 60.41 | | C |
| ANISOU | 1323 | C | LEU | C | 180 | 9146 | 7766 | 6040 | −3384 | 222 | 1197 | C |
| ATOM | 1324 | O | LEU | C | 180 | 64.924 | 7.490 | −5.575 | 1.00 | 37.38 | | O |
| ANISOU | 1324 | O | LEU | C | 180 | 6089 | 4892 | 3222 | −3220 | 305 | 1127 | O |
| ATOM | 1325 | CB | LEU | C | 180 | 65.522 | 7.659 | −2.590 | 1.00 | 51.24 | | C |
| ANISOU | 1325 | CB | LEU | C | 180 | 7944 | 6714 | 4809 | −3525 | 98 | 1238 | C |
| ATOM | 1326 | CG | LEU | C | 180 | 65.019 | 8.659 | −1.599 | 1.00 | 55.30 | | C |
| ANISOU | 1326 | CG | LEU | C | 180 | 8365 | 7428 | 5220 | −3623 | 107 | 1131 | C |
| ATOM | 1327 | CD1 | LEU | C | 180 | 64.355 | 7.985 | −0.447 | 1.00 | 57.45 | | C |
| ANISOU | 1327 | CD1 | LEU | C | 180 | 8745 | 7827 | 5256 | −3880 | 61 | 1151 | C |
| ATOM | 1328 | CD2 | LEU | C | 180 | 66.132 | 9.426 | −1.045 | 1.00 | 57.86 | | C |
| ANISOU | 1328 | CD2 | LEU | C | 180 | 8617 | 7693 | 5675 | −3526 | 26 | 1170 | C |
| ATOM | 1329 | N | GLY | C | 181 | 63.938 | 5.316 | −6.973 | 1.00 | 77.17 | | N |
| ANISOU | 1329 | N | GLY | C | 181 | 11303 | 9815 | 8204 | −3231 | 349 | 1097 | N |
| ATOM | 1330 | CA | GLY | C | 181 | 65.132 | 4.853 | −7.665 | 1.00 | 77.11 | | C |
| ANISOU | 1330 | CA | GLY | C | 181 | 11336 | 9607 | 8354 | −3043 | 273 | 1204 | C |
| ATOM | 1331 | C | GLY | C | 181 | 66.258 | 5.862 | −7.785 | 1.00 | 78.65 | | C |
| ANISOU | 1331 | C | GLY | C | 181 | 11386 | 9796 | 8700 | −2858 | 277 | 1253 | C |
| ATOM | 1332 | O | GLY | C | 181 | 66.083 | 6.933 | −8.369 | 1.00 | 76.90 | | O |
| ANISOU | 1332 | O | GLY | C | 181 | 11019 | 9679 | 8521 | −2763 | 379 | 1192 | O |
| ATOM | 1333 | N | MET | C | 182 | 67.407 | 5.534 | −7.176 | 1.00 | 75.11 | | N |
| ANISOU | 1333 | N | MET | C | 182 | 10985 | 9227 | 8326 | −2821 | 154 | 1366 | N |
| ATOM | 1334 | CA | MET | C | 182 | 68.687 | 6.242 | −7.262 | 1.00 | 74.45 | | C |
| ANISOU | 1334 | CA | MET | C | 182 | 10767 | 9127 | 8395 | −2661 | 131 | 1425 | C |
| ATOM | 1335 | C | MET | C | 182 | 69.651 | 5.096 | −7.564 | 1.00 | 82.01 | | C |
| ANISOU | 1335 | C | MET | C | 182 | 11804 | 9929 | 9428 | −2521 | 17 | 1494 | C |
| ATOM | 1336 | O | MET | C | 182 | 70.408 | 4.646 | −6.699 | 1.00 | 82.92 | | O |
| ANISOU | 1336 | O | MET | C | 182 | 11991 | 9959 | 9554 | −2533 | −126 | 1574 | O |
| ATOM | 1337 | CB | MET | C | 182 | 69.064 | 6.981 | −5.966 | 1.00 | 76.30 | | C |
| ANISOU | 1337 | CB | MET | C | 182 | 10967 | 9408 | 8615 | −2774 | 66 | 1459 | C |
| ATOM | 1338 | CG | MET | C | 182 | 68.149 | 8.130 | −5.634 | 1.00 | 78.35 | | C |
| ANISOU | 1338 | CG | MET | C | 182 | 11154 | 9818 | 8797 | −2890 | 152 | 1361 | C |
| ATOM | 1339 | SD | MET | C | 182 | 68.962 | 9.492 | −4.774 | 1.00 | 81.42 | | S |
| ANISOU | 1339 | SD | MET | C | 182 | 11408 | 10256 | 9272 | −2899 | 111 | 1361 | S |
| ATOM | 1340 | CE | MET | C | 182 | 69.158 | 8.821 | −3.206 | 1.00 | 79.38 | | C |
| ANISOU | 1340 | CE | MET | C | 182 | 11270 | 9975 | 8915 | −3040 | −51 | 1434 | C |
| ATOM | 1341 | N | GLY | C | 183 | 69.509 | 4.571 | −8.778 | 1.00 | 79.70 | | N |
| ANISOU | 1341 | N | GLY | C | 183 | 11509 | 9607 | 9167 | −2381 | 70 | 1446 | N |
| ATOM | 1342 | CA | GLY | C | 183 | 70.228 | 3.416 | −9.300 | 1.00 | 80.97 | | C |
| ANISOU | 1342 | CA | GLY | C | 183 | 11746 | 9628 | 9392 | −2211 | −33 | 1461 | C |
| ATOM | 1343 | C | GLY | C | 183 | 71.735 | 3.511 | −9.343 | 1.00 | 84.88 | | C |
| ANISOU | 1343 | C | GLY | C | 183 | 12121 | 10114 | 10015 | −2013 | −106 | 1512 | C |
| ATOM | 1344 | O | GLY | C | 183 | 72.404 | 2.597 | −8.853 | 1.00 | 86.39 | | O |
| ANISOU | 1344 | O | GLY | C | 183 | 12428 | 10169 | 10226 | −1950 | −276 | 1555 | O |
| ATOM | 1345 | N | GLN | C | 184 | 72.281 | 4.599 | −9.945 | 1.00 | 79.06 | | N |
| ANISOU | 1345 | N | GLN | C | 184 | 11153 | 9526 | 9362 | −1915 | 12 | 1507 | N |
| ATOM | 1346 | CA | GLN | C | 184 | 73.728 | 4.807 | −10.045 | 1.00 | 79.33 | | C |
| ANISOU | 1346 | CA | GLN | C | 184 | 11023 | 9608 | 9512 | −1751 | −35 | 1545 | C |
| ATOM | 1347 | C | GLN | C | 184 | 74.347 | 4.814 | −8.649 | 1.00 | 82.34 | | C |
| ANISOU | 1347 | C | GLN | C | 184 | 11448 | 9935 | 9901 | −1840 | −187 | 1612 | C |
| ATOM | 1348 | O | GLN | C | 184 | 75.422 | 4.241 | −8.451 | 1.00 | 84.14 | | O |
| ANISOU | 1348 | O | GLN | C | 184 | 11647 | 10131 | 10189 | −1694 | −318 | 1630 | O |
| ATOM | 1349 | CB | GLN | C | 184 | 74.054 | 6.107 | −10.796 | 1.00 | 80.49 | | C |
| ANISOU | 1349 | CB | GLN | C | 184 | 10931 | 9925 | 9725 | −1716 | 129 | 1552 | C |
| ATOM | 1350 | CG | GLN | C | 184 | 73.818 | 6.043 | −12.314 | 1.00 | 107.74 | | C |
| ANISOU | 1350 | CG | GLN | C | 184 | 14303 | 13465 | 13170 | −1567 | 266 | 1499 | C |
| ATOM | 1351 | CD | GLN | C | 184 | 74.889 | 5.280 | −13.065 | 1.00 | 131.97 | | C |
| ANISOU | 1351 | CD | GLN | C | 184 | 17267 | 16582 | 16294 | −1324 | 226 | 1469 | C |
| ATOM | 1352 | NE2 | GLN | C | 184 | 75.995 | 5.952 | −13.367 | 1.00 | 124.57 | | N |
| ANISOU | 1352 | NE2 | GLN | C | 184 | 16101 | 15798 | 15432 | −1254 | 280 | 1510 | N |
| ATOM | 1353 | OE1 | GLN | C | 184 | 74.726 | 4.096 | −13.402 | 1.00 | 127.85 | | O |
| ANISOU | 1353 | OE1 | GLN | C | 184 | 16862 | 15969 | 15744 | −1200 | 145 | 1398 | O |
| ATOM | 1354 | N | LEU | C | 185 | 73.617 | 5.402 | −7.673 | 1.00 | 75.35 | | N |
| ANISOU | 1354 | N | LEU | C | 185 | 10636 | 9055 | 8937 | −2065 | −178 | 1634 | N |
| ATOM | 1355 | CA | LEU | C | 185 | 73.995 | 5.471 | −6.273 | 1.00 | 74.30 | | C |
| ANISOU | 1355 | CA | LEU | C | 185 | 10560 | 8896 | 8774 | −2178 | −315 | 1690 | C |
| ATOM | 1356 | C | LEU | C | 185 | 73.880 | 4.100 | −5.610 | 1.00 | 77.67 | | C |
| ANISOU | 1356 | C | LEU | C | 185 | 11238 | 9159 | 9115 | −2199 | −489 | 1737 | C |
| ATOM | 1357 | O | LEU | C | 185 | 74.882 | 3.630 | −5.070 | 1.00 | 78.89 | | O |
| ANISOU | 1357 | O | LEU | C | 185 | 11415 | 9254 | 9305 | −2098 | −652 | 1785 | O |
| ATOM | 1358 | CB | LEU | C | 185 | 73.142 | 6.515 | −5.541 | 1.00 | 73.19 | | C |
| ANISOU | 1358 | CB | LEU | C | 185 | 10410 | 8840 | 8557 | −2398 | −242 | 1670 | C |
| ATOM | 1359 | CG | LEU | C | 185 | 73.773 | 7.113 | −4.281 | 1.00 | 78.53 | | C |
| ANISOU | 1359 | CG | LEU | C | 185 | 11037 | 9561 | 9240 | −2485 | −342 | 1700 | C |
| ATOM | 1360 | CD1 | LEU | C | 185 | 73.995 | 8.618 | −4.418 | 1.00 | 77.44 | | C |
| ANISOU | 1360 | CD1 | LEU | C | 185 | 10697 | 9533 | 9196 | −2513 | −239 | 1658 | C |
| ATOM | 1361 | CD2 | LEU | C | 185 | 72.926 | 6.826 | −3.073 | 1.00 | 82.34 | | C |
| ANISOU | 1361 | CD2 | LEU | C | 185 | 11697 | 10039 | 9550 | −2689 | −412 | 1713 | C |
| ATOM | 1362 | N | LYS | C | 186 | 72.682 | 3.445 | −5.671 | 1.00 | 72.77 | | N |
| ANISOU | 1362 | N | LYS | C | 186 | 10808 | 8462 | 8380 | −2327 | −463 | 1724 | N |
| ATOM | 1363 | CA | LYS | C | 186 | 72.413 | 2.126 | −5.065 | 1.00 | 73.66 | | C |
| ANISOU | 1363 | CA | LYS | C | 186 | 11202 | 8388 | 8398 | −2401 | −625 | 1788 | C |
| ATOM | 1364 | C | LYS | C | 186 | 73.388 | 1.050 | −5.548 | 1.00 | 80.72 | | C |
| ANISOU | 1364 | C | LYS | C | 186 | 12167 | 9116 | 9387 | −2145 | −781 | 1797 | C |
| ATOM | 1365 | O | LYS | C | 186 | 73.778 | 0.198 | −4.756 | 1.00 | 81.08 | | O |
| ANISOU | 1365 | O | LYS | C | 186 | 12409 | 9003 | 9395 | −2142 | −981 | 1880 | O |
| ATOM | 1366 | CB | LYS | C | 186 | 70.952 | 1.688 | −5.258 | 1.00 | 74.70 | | C |
| ANISOU | 1366 | CB | LYS | C | 186 | 11485 | 8495 | 8403 | −2603 | −544 | 1756 | C |
| ATOM | 1367 | CG | LYS | C | 186 | 70.501 | 0.599 | −4.290 | 1.00 | 86.54 | | C |
| ANISOU | 1367 | CG | LYS | C | 186 | 13286 | 9829 | 9766 | −2796 | −697 | 1854 | C |
| ATOM | 1368 | CD | LYS | C | 186 | 69.607 | −0.469 | −4.944 | 1.00 | 98.54 | | C |
| ANISOU | 1368 | CD | LYS | C | 186 | 14982 | 11224 | 11232 | −2886 | −679 | 1816 | C |
| ATOM | 1369 | CE | LYS | C | 186 | 69.608 | −1.806 | −4.202 | 1.00 | 116.18 | | C |
| ANISOU | 1369 | CE | LYS | C | 186 | 17554 | 13201 | 13387 | −3007 | −884 | 1938 | C |
| ATOM | 1370 | NZ | LYS | C | 186 | 68.772 | −1.825 | −2.956 | 1.00 | 119.77 | | N |
| ANISOU | 1370 | NZ | LYS | C | 186 | 18152 | 13714 | 13641 | −3351 | −907 | 2047 | N |
| ATOM | 1371 | N | TRP | C | 187 | 73.806 | 1.117 | −6.836 | 1.00 | 79.95 | | N |
| ANISOU | 1371 | N | TRP | C | 187 | 11905 | 9068 | 9403 | −1917 | −698 | 1709 | N |
| ATOM | 1372 | CA | TRP | C | 187 | 74.796 | 0.217 | −7.445 | 1.00 | 82.50 | | C |
| ANISOU | 1372 | CA | TRP | C | 187 | 12236 | 9289 | 9822 | −1623 | −832 | 1671 | C |
| ATOM | 1373 | C | TRP | C | 187 | 76.131 | 0.459 | −6.756 | 1.00 | 83.18 | | C |
| ANISOU | 1373 | C | TRP | C | 187 | 12206 | 9427 | 9972 | −1495 | −962 | 1713 | C |
| ATOM | 1374 | O | TRP | C | 187 | 76.678 | −0.473 | −6.164 | 1.00 | 85.49 | | O |
| ANISOU | 1374 | O | TRP | C | 187 | 12669 | 9555 | 10259 | −1395 | −1186 | 1754 | O |
| ATOM | 1375 | CB | TRP | C | 187 | 74.885 | 0.420 | −8.988 | 1.00 | 82.21 | | C |
| ANISOU | 1375 | CB | TRP | C | 187 | 12002 | 9372 | 9861 | −1427 | −678 | 1554 | C |
| ATOM | 1376 | CG | TRP | C | 187 | 76.156 | −0.022 | −9.683 | 1.00 | 86.06 | | C |
| ANISOU | 1376 | CG | TRP | C | 187 | 12347 | 9897 | 10455 | −1094 | −760 | 1484 | C |
| ATOM | 1377 | CD1 | TRP | C | 187 | 77.013 | −1.012 | −9.294 | 1.00 | 91.50 | | C |
| ANISOU | 1377 | CD1 | TRP | C | 187 | 13148 | 10439 | 11180 | −902 | −996 | 1479 | C |
| ATOM | 1378 | CD2 | TRP | C | 187 | 76.646 | 0.452 | −10.952 | 1.00 | 86.22 | | C |
| ANISOU | 1378 | CD2 | TRP | C | 187 | 12090 | 10129 | 10539 | −905 | −610 | 1396 | C |
| ATOM | 1379 | CE2 | TRP | C | 187 | 77.836 | −0.260 | −11.236 | 1.00 | 92.54 | | C |
| ANISOU | 1379 | CE2 | TRP | C | 187 | 12816 | 10938 | 11409 | −605 | −756 | 1326 | C |
| ATOM | 1380 | CE3 | TRP | C | 187 | 76.204 | 1.424 | −11.873 | 1.00 | 86.35 | | C |
| ANISOU | 1380 | CE3 | TRP | C | 187 | 11918 | 10343 | 10550 | −956 | −371 | 1373 | C |
| ATOM | 1381 | NE1 | TRP | C | 187 | 78.050 | −1.127 | −10.194 | 1.00 | 92.49 | | N |
| ANISOU | 1381 | NE1 | TRP | C | 187 | 13049 | 10702 | 11391 | −595 | −999 | 1373 | N |
| ATOM | 1382 | CZ2 | TRP | C | 187 | 78.587 | −0.034 | −12.400 | 1.00 | 92.60 | | C |
| ANISOU | 1382 | CZ2 | TRP | C | 187 | 12542 | 11176 | 11467 | −374 | −653 | 1229 | C |
| ATOM | 1383 | CZ3 | TRP | C | 187 | 76.945 | 1.645 | −13.030 | 1.00 | 88.53 | | C |
| ANISOU | 1383 | CZ3 | TRP | C | 187 | 11944 | 10821 | 10875 | −739 | −274 | 1305 | C |
| ATOM | 1384 | CH2 | TRP | C | 187 | 78.124 | 0.927 | −13.282 | 1.00 | 91.33 | | C |
| ANISOU | 1384 | CH2 | TRP | C | 187 | 12203 | 11212 | 11285 | −461 | −404 | 1231 | C |
| ATOM | 1385 | N | GLU | C | 188 | 76.619 | 1.708 | −6.781 | 1.00 | 74.50 | | N |
| ANISOU | 1385 | N | GLU | C | 188 | 10833 | 8544 | 8929 | −1512 | −838 | 1706 | N |
| ATOM | 1386 | CA | GLU | C | 188 | 77.882 | 2.052 | −6.152 | 1.00 | 73.86 | | C |
| ANISOU | 1386 | CA | GLU | C | 188 | 10601 | 8550 | 8912 | −1413 | −946 | 1728 | C |
| ATOM | 1387 | C | GLU | C | 188 | 77.928 | 1.682 | −4.659 | 1.00 | 78.08 | | C |
| ANISOU | 1387 | C | GLU | C | 188 | 11338 | 8970 | 9359 | −1533 | −1143 | 1823 | C |
| ATOM | 1388 | O | GLU | C | 188 | 78.900 | 1.047 | −4.238 | 1.00 | 79.51 | | O |
| ANISOU | 1388 | O | GLU | C | 188 | 11552 | 9091 | 9565 | −1354 | −1347 | 1835 | O |
| ATOM | 1389 | CB | GLU | C | 188 | 78.239 | 3.508 | −6.398 | 1.00 | 73.35 | | C |
| ANISOU | 1389 | CB | GLU | C | 188 | 10234 | 8715 | 8919 | −1474 | −773 | 1710 | C |
| ATOM | 1390 | CG | GLU | C | 188 | 79.732 | 3.685 | −6.555 | 1.00 | 80.04 | | C |
| ANISOU | 1390 | CG | GLU | C | 188 | 10833 | 9705 | 9875 | −1272 | −836 | 1676 | C |
| ATOM | 1391 | CD | GLU | C | 188 | 80.204 | 5.099 | −6.811 | 1.00 | 100.47 | | C |
| ANISOU | 1391 | CD | GLU | C | 188 | 13125 | 12507 | 12544 | −1359 | −680 | 1672 | C |
| ATOM | 1392 | OE1 | GLU | C | 188 | 79.391 | 5.941 | −7.264 | 1.00 | 83.11 | | O |
| ANISOU | 1392 | OE1 | GLU | C | 188 | 10896 | 10345 | 10337 | −1511 | −495 | 1683 | O |
| ATOM | 1393 | OE2 | GLU | C | 188 | 81.408 | 5.354 | −6.583 | 1.00 | 101.97 | | O |
| ANISOU | 1393 | OE2 | GLU | C | 188 | 13112 | 12826 | 12805 | −1269 | −751 | 1654 | O |
| ATOM | 1394 | N | LEU | C | 189 | 76.858 | 1.998 | −3.884 | 1.00 | 72.86 | | N |
| ANISOU | 1394 | N | LEU | C | 189 | 10816 | 8289 | 8577 | −1819 | −1092 | 1882 | N |
| ATOM | 1395 | CA | LEU | C | 189 | 76.779 | 1.649 | −2.452 | 1.00 | 73.29 | | C |
| ANISOU | 1395 | CA | LEU | C | 189 | 11074 | 8265 | 8507 | −1965 | −1262 | 1984 | C |
| ATOM | 1396 | C | LEU | C | 189 | 76.922 | 0.135 | −2.254 | 1.00 | 77.98 | | C |
| ANISOU | 1396 | C | LEU | C | 189 | 11966 | 8607 | 9056 | −1859 | −1488 | 2050 | C |
| ATOM | 1397 | O | LEU | C | 189 | 77.725 | −0.283 | −1.421 | 1.00 | 79.35 | | O |
| ANISOU | 1397 | O | LEU | C | 189 | 12220 | 8721 | 9208 | −1771 | −1699 | 2113 | O |
| ATOM | 1398 | CB | LEU | C | 189 | 75.465 | 2.127 | −1.788 | 1.00 | 71.93 | | C |
| ANISOU | 1398 | CB | LEU | C | 189 | 11004 | 8142 | 8184 | −2290 | −1151 | 2016 | C |
| ATOM | 1399 | CG | LEU | C | 189 | 75.241 | 3.620 | −1.557 | 1.00 | 74.40 | | C |
| ANISOU | 1399 | CG | LEU | C | 189 | 11093 | 8670 | 8505 | −2426 | −994 | 1959 | C |
| ATOM | 1400 | CD1 | LEU | C | 189 | 73.845 | 3.859 | −1.038 | 1.00 | 73.42 | | C |
| ANISOU | 1400 | CD1 | LEU | C | 189 | 11088 | 8597 | 8212 | −2705 | −904 | 1961 | C |
| ATOM | 1401 | CD2 | LEU | C | 189 | 76.229 | 4.185 | −0.554 | 1.00 | 77.85 | | C |
| ANISOU | 1401 | CD2 | LEU | C | 189 | 11416 | 9199 | 8965 | −2404 | −1112 | 1978 | C |
| ATOM | 1402 | N | GLU | C | 190 | 76.165 | −0.673 | −3.049 | 1.00 | 72.83 | | N |
| ANISOU | 1402 | N | GLU | C | 190 | 11478 | 7800 | 8395 | −1856 | −1456 | 2028 | N |
| ATOM | 1403 | CA | GLU | C | 190 | 76.158 | −2.133 | −3.015 | 1.00 | 73.86 | | C |
| ANISOU | 1403 | CA | GLU | C | 190 | 11924 | 7642 | 8498 | −1770 | −1669 | 2079 | C |
| ATOM | 1404 | C | GLU | C | 190 | 77.527 | −2.717 | −3.282 | 1.00 | 78.97 | | C |
| ANISOU | 1404 | C | GLU | C | 190 | 12520 | 8221 | 9266 | −1397 | −1865 | 2030 | C |
| ATOM | 1405 | O | GLU | C | 190 | 78.016 | −3.497 | −2.466 | 1.00 | 81.01 | | O |
| ANISOU | 1405 | O | GLU | C | 190 | 12992 | 8307 | 9481 | −1323 | −2118 | 2117 | O |
| ATOM | 1406 | CB | GLU | C | 190 | 75.155 | −2.696 | −4.020 | 1.00 | 74.51 | | C |
| ANISOU | 1406 | CB | GLU | C | 190 | 12122 | 7610 | 8578 | −1825 | −1568 | 2019 | C |
| ATOM | 1407 | CG | GLU | C | 190 | 73.779 | −2.937 | −3.429 | 1.00 | 84.95 | | C |
| ANISOU | 1407 | CG | GLU | C | 190 | 13680 | 8864 | 9735 | −2195 | −1520 | 2107 | C |
| ATOM | 1408 | CD | GLU | C | 190 | 72.884 | −3.872 | −4.223 | 1.00 | 105.30 | | C |
| ANISOU | 1408 | CD | GLU | C | 190 | 16447 | 11260 | 12301 | −2256 | −1501 | 2061 | C |
| ATOM | 1409 | OE1 | GLU | C | 190 | 73.191 | −4.142 | −5.408 | 1.00 | 92.28 | | O |
| ANISOU | 1409 | OE1 | GLU | C | 190 | 14710 | 9573 | 10778 | −2002 | −1482 | 1934 | O |
| ATOM | 1410 | OE2 | GLU | C | 190 | 71.876 | −4.348 | −3.650 | 1.00 | 101.96 | | O |
| ANISOU | 1410 | OE2 | GLU | C | 190 | 16255 | 10750 | 11735 | −2567 | −1506 | 2146 | O |
| ATOM | 1411 | N | ASP | C | 191 | 78.152 | −2.331 | −4.411 | 1.00 | 74.44 | | N |
| ANISOU | 1411 | N | ASP | C | 191 | 11659 | 7797 | 8827 | −1158 | −1754 | 1888 | N |
| ATOM | 1412 | CA | ASP | C | 191 | 79.473 | −2.809 | −4.832 | 1.00 | 76.29 | | C |
| ANISOU | 1412 | CA | ASP | C | 191 | 11772 | 8041 | 9173 | −777 | −1910 | 1794 | C |
| ATOM | 1413 | C | ASP | C | 191 | 80.587 | −2.439 | −3.840 | 1.00 | 79.90 | | C |
| ANISOU | 1413 | C | ASP | C | 191 | 12113 | 8608 | 9638 | −692 | −2059 | 1834 | C |
| ATOM | 1414 | O | ASP | C | 191 | 81.458 | −3.270 | −3.556 | 1.00 | 81.37 | | O |
| ANISOU | 1414 | O | ASP | C | 191 | 12399 | 8674 | 9844 | −437 | −2318 | 1823 | O |
| ATOM | 1415 | CB | ASP | C | 191 | 79.806 | −2.321 | −6.260 | 1.00 | 77.51 | | C |
| ANISOU | 1415 | CB | ASP | C | 191 | 11596 | 8418 | 9437 | −600 | −1708 | 1641 | C |
| ATOM | 1416 | CG | ASP | C | 191 | 79.030 | −2.976 | −7.406 | 1.00 | 87.87 | | C |
| ANISOU | 1416 | CG | ASP | C | 191 | 13008 | 9620 | 10760 | −537 | −1634 | 1551 | C |
| ATOM | 1417 | OD1 | ASP | C | 191 | 77.927 | −3.523 | −7.150 | 1.00 | 88.55 | | O |
| ANISOU | 1417 | OD1 | ASP | C | 191 | 13384 | 9493 | 10767 | −744 | −1647 | 1612 | O |
| ATOM | 1418 | OD2 | ASP | C | 191 | 79.499 | −2.886 | −8.574 | 1.00 | 91.41 | | O |
| ANISOU | 1418 | OD2 | ASP | C | 191 | 13221 | 10227 | 11285 | −302 | −1545 | 1414 | O |
| ATOM | 1419 | N | LEU | C | 192 | 80.543 | −1.214 | −3.294 | 1.00 | 74.62 | | N |
| ANISOU | 1419 | N | LEU | C | 192 | 11246 | 8157 | 8949 | −898 | −1915 | 1871 | N |
| ATOM | 1420 | CA | LEU | C | 192 | 81.538 | −0.775 | −2.317 | 1.00 | 75.99 | | C |
| ANISOU | 1420 | CA | LEU | C | 192 | 11291 | 8458 | 9124 | −851 | −2046 | 1896 | C |
| ATOM | 1421 | C | LEU | C | 192 | 81.361 | −1.471 | −0.959 | 1.00 | 81.25 | | C |
| ANISOU | 1421 | C | LEU | C | 192 | 12293 | 8929 | 9651 | −951 | −2283 | 2040 | C |
| ATOM | 1422 | O | LEU | C | 192 | 82.339 | −1.653 | −0.230 | 1.00 | 81.97 | | O |
| ANISOU | 1422 | O | LEU | C | 192 | 12362 | 9046 | 9735 | −798 | −2493 | 2053 | O |
| ATOM | 1423 | CB | LEU | C | 192 | 81.537 | 0.755 | −2.141 | 1.00 | 74.27 | | C |
| ANISOU | 1423 | CB | LEU | C | 192 | 10775 | 8510 | 8935 | −1050 | −1835 | 1878 | C |
| ATOM | 1424 | CG | LEU | C | 192 | 81.978 | 1.641 | −3.313 | 1.00 | 78.01 | | C |
| ANISOU | 1424 | CG | LEU | C | 192 | 10887 | 9212 | 9541 | −973 | −1616 | 1767 | C |
| ATOM | 1425 | CD1 | LEU | C | 192 | 81.920 | 3.104 | −2.908 | 1.00 | 76.84 | | C |
| ANISOU | 1425 | CD1 | LEU | C | 192 | 10522 | 9259 | 9415 | −1202 | −1462 | 1776 | C |
| ATOM | 1426 | CD2 | LEU | C | 192 | 83.382 | 1.273 | −3.838 | 1.00 | 81.88 | | C |
| ANISOU | 1426 | CD2 | LEU | C | 192 | 11157 | 9821 | 10133 | −629 | −1724 | 1660 | C |
| ATOM | 1427 | N | SER | C | 193 | 80.106 | −1.837 | −0.621 | 1.00 | 77.72 | | N |
| ANISOU | 1427 | N | SER | C | 193 | 12144 | 8309 | 9077 | −1218 | −2247 | 2148 | N |
| ATOM | 1428 | CA | SER | C | 193 | 79.762 | −2.550 | 0.607 | 1.00 | 79.25 | | C |
| ANISOU | 1428 | CA | SER | C | 193 | 12689 | 8316 | 9106 | −1366 | −2450 | 2313 | C |
| ATOM | 1429 | C | SER | C | 193 | 80.324 | −3.960 | 0.517 | 1.00 | 88.29 | | C |
| ANISOU | 1429 | C | SER | C | 193 | 14103 | 9170 | 10271 | −1093 | −2744 | 2342 | C |
| ATOM | 1430 | O | SER | C | 193 | 80.984 | −4.402 | 1.453 | 1.00 | 90.68 | | O |
| ANISOU | 1430 | O | SER | C | 193 | 14546 | 9398 | 10510 | −993 | −2996 | 2428 | O |
| ATOM | 1431 | CB | SER | C | 193 | 78.253 | −2.588 | 0.803 | 1.00 | 79.67 | | C |
| ANISOU | 1431 | CB | SER | C | 193 | 12952 | 8301 | 9017 | −1736 | −2307 | 2402 | C |
| ATOM | 1432 | OG | SER | C | 193 | 77.763 | −1.297 | 1.113 | 1.00 | 81.79 | | O |
| ANISOU | 1432 | OG | SER | C | 193 | 13005 | 8832 | 9240 | −1971 | −2093 | 2376 | O |
| ATOM | 1433 | N | PHE | C | 194 | 80.125 | −4.626 | −0.642 | 1.00 | 85.99 | | N |
| ANISOU | 1433 | N | PHE | C | 194 | 13869 | 8728 | 10076 | −939 | −2723 | 2253 | N |
| ATOM | 1434 | CA | PHE | C | 194 | 80.639 | −5.959 | −0.964 | 1.00 | 89.33 | | C |
| ANISOU | 1434 | CA | PHE | C | 194 | 14533 | 8858 | 10552 | −632 | −2999 | 2230 | C |
| ATOM | 1435 | C | PHE | C | 194 | 82.177 | −5.967 | −0.871 | 1.00 | 95.47 | | C |
| ANISOU | 1435 | C | PHE | C | 194 | 15096 | 9760 | 11419 | −238 | −3188 | 2131 | C |
| ATOM | 1436 | O | PHE | C | 194 | 82.742 | −6.867 | −0.247 | 1.00 | 97.41 | | O |
| ANISOU | 1436 | O | PHE | C | 194 | 15585 | 9798 | 11630 | −49 | −3504 | 2193 | O |
| ATOM | 1437 | CB | PHE | C | 194 | 80.149 | −6.376 | −2.373 | 1.00 | 90.65 | | C |
| ANISOU | 1437 | CB | PHE | C | 194 | 14691 | 8933 | 10820 | −535 | −2883 | 2094 | C |
| ATOM | 1438 | CG | PHE | C | 194 | 80.515 | −7.746 | −2.919 | 1.00 | 95.44 | | C |
| ANISOU | 1438 | CG | PHE | C | 194 | 15545 | 9220 | 11497 | −217 | −3146 | 2024 | C |
| ATOM | 1439 | CD1 | PHE | C | 194 | 80.733 | −8.825 | −2.064 | 1.00 | 102.04 | | C |
| ANISOU | 1439 | CD1 | PHE | C | 194 | 16767 | 9731 | 12274 | −134 | −3490 | 2151 | C |
| ATOM | 1440 | CD2 | PHE | C | 194 | 80.564 | −7.973 | −4.289 | 1.00 | 97.30 | | C |
| ANISOU | 1440 | CD2 | PHE | C | 194 | 15656 | 9464 | 11848 | −8 | −3058 | 1834 | C |
| ATOM | 1441 | CE1 | PHE | C | 194 | 81.066 | −10.086 | −2.570 | 1.00 | 105.87 | | C |
| ANISOU | 1441 | CE1 | PHE | C | 194 | 17503 | 9886 | 12835 | 178 | −3758 | 2073 | C |
| ATOM | 1442 | CE2 | PHE | C | 194 | 80.883 | −9.238 | −4.794 | 1.00 | 103.09 | | C |
| ANISOU | 1442 | CE2 | PHE | C | 194 | 16623 | 9897 | 12651 | 302 | −3316 | 1741 | C |
| ATOM | 1443 | CZ | PHE | C | 194 | 81.131 | −10.285 | −3.931 | 1.00 | 104.58 | | C |
| ANISOU | 1443 | CZ | PHE | C | 194 | 17197 | 9740 | 12797 | 396 | −3670 | 1858 | C |
| ATOM | 1444 | N | ARG | C | 195 | 82.834 | −4.922 | −1.433 | 1.00 | 91.52 | | N |
| ANISOU | 1444 | N | ARG | C | 195 | 14141 | 9610 | 11021 | −140 | −2997 | 1985 | N |
| ATOM | 1445 | CA | ARG | C | 195 | 84.289 | −4.712 | −1.419 | 1.00 | 93.22 | | C |
| ANISOU | 1445 | CA | ARG | C | 195 | 14056 | 10042 | 11321 | 188 | −3117 | 1864 | C |
| ATOM | 1446 | C | ARG | C | 195 | 84.892 | −4.852 | 0.006 | 1.00 | 100.62 | | C |
| ANISOU | 1446 | C | ARG | C | 195 | 15118 | 10952 | 12163 | 206 | −3377 | 1976 | C |
| ATOM | 1447 | O | ARG | C | 195 | 85.953 | −5.464 | 0.172 | 1.00 | 102.81 | | O |
| ANISOU | 1447 | O | ARG | C | 195 | 15386 | 11206 | 12472 | 557 | −3644 | 1909 | O |
| ATOM | 1448 | CB | ARG | C | 195 | 84.633 | −3.340 | −2.052 | 1.00 | 89.06 | | C |
| ANISOU | 1448 | CB | ARG | C | 195 | 13046 | 9909 | 10884 | 125 | −2817 | 1749 | C |
| ATOM | 1449 | CG | ARG | C | 195 | 86.128 | −3.026 | −2.141 | 1.00 | 96.46 | | C |
| ANISOU | 1449 | CG | ARG | C | 195 | 13610 | 11128 | 11910 | 422 | −2899 | 1605 | C |
| ATOM | 1450 | CD | ARG | C | 195 | 86.408 | −1.649 | −2.707 | 1.00 | 102.16 | | C |
| ANISOU | 1450 | CD | ARG | C | 195 | 13891 | 12213 | 12712 | 292 | −2601 | 1527 | C |
| ATOM | 1451 | NE | ARG | C | 195 | 86.663 | −1.684 | −4.149 | 1.00 | 110.80 | | N |
| ANISOU | 1451 | NE | ARG | C | 195 | 14753 | 13449 | 13899 | 486 | −2455 | 1377 | N |
| ATOM | 1452 | CZ | ARG | C | 195 | 87.873 | −1.721 | −4.702 | 1.00 | 123.35 | | C |
| ANISOU | 1452 | CZ | ARG | C | 195 | 16022 | 15284 | 15561 | 794 | −2507 | 1214 | C |
| ATOM | 1453 | NH1 | ARG | C | 195 | 88.961 | −1.728 | −3.939 | 1.00 | 116.01 | | N |
| ANISOU | 1453 | NH1 | ARG | C | 195 | 14960 | 14483 | 14636 | 954 | −2711 | 1170 | N |
| ATOM | 1454 | NH2 | ARG | C | 195 | 88.006 | −1.751 | −6.021 | 1.00 | 101.01 | | N |
| ANISOU | 1454 | NH2 | ARG | C | 195 | 12988 | 12604 | 12786 | 946 | −2357 | 1083 | N |
| ATOM | 1455 | N | TYR | C | 196 | 84.191 | −4.318 | 1.019 | 1.00 | 97.05 | | N |
| ANISOU | 1455 | N | TYR | C | 196 | 14785 | 10512 | 11578 | −155 | −3310 | 2137 | N |
| ATOM | 1456 | CA | TYR | C | 196 | 84.651 | −4.348 | 2.401 | 1.00 | 99.29 | | C |
| ANISOU | 1456 | CA | TYR | C | 196 | 15179 | 10805 | 11742 | −177 | −3531 | 2250 | C |
| ATOM | 1457 | C | TYR | C | 196 | 83.832 | −5.288 | 3.312 | 1.00 | 103.50 | | C |
| ANISOU | 1457 | C | TYR | C | 196 | 16225 | 11008 | 12091 | −365 | −3713 | 2476 | C |
| ATOM | 1458 | O | TYR | C | 196 | 84.151 | −5.401 | 4.499 | 1.00 | 104.95 | | O |
| ANISOU | 1458 | O | TYR | C | 196 | 16543 | 11194 | 12141 | −402 | −3903 | 2598 | O |
| ATOM | 1459 | CB | TYR | C | 196 | 84.734 | −2.919 | 2.972 | 1.00 | 99.53 | | C |
| ANISOU | 1459 | CB | TYR | C | 196 | 14891 | 11173 | 11751 | −408 | −3341 | 2232 | C |
| ATOM | 1460 | CG | TYR | C | 196 | 85.644 | −1.998 | 2.182 | 1.00 | 102.03 | | C |
| ANISOU | 1460 | CG | TYR | C | 196 | 14719 | 11807 | 12241 | −256 | −3184 | 2036 | C |
| ATOM | 1461 | CD1 | TYR | C | 196 | 87.030 | −2.074 | 2.309 | 1.00 | 106.70 | | C |
| ANISOU | 1461 | CD1 | TYR | C | 196 | 15081 | 12559 | 12903 | 66 | −3368 | 1920 | C |
| ATOM | 1462 | CD2 | TYR | C | 196 | 85.120 | −1.043 | 1.318 | 1.00 | 100.47 | | C |
| ANISOU | 1462 | CD2 | TYR | C | 196 | 14287 | 11761 | 12126 | −442 | −2856 | 1971 | C |
| ATOM | 1463 | CE1 | TYR | C | 196 | 87.872 | −1.228 | 1.584 | 1.00 | 107.50 | | C |
| ANISOU | 1463 | CE1 | TYR | C | 196 | 14717 | 12979 | 13149 | 166 | −3213 | 1748 | C |
| ATOM | 1464 | CE2 | TYR | C | 196 | 85.951 | −0.180 | 0.601 | 1.00 | 101.20 | | C |
| ANISOU | 1464 | CE2 | TYR | C | 196 | 13948 | 12142 | 12363 | −341 | −2709 | 1820 | C |
| ATOM | 1465 | CZ | TYR | C | 196 | 87.328 | −0.278 | 0.734 | 1.00 | 112.28 | | C |
| ANISOU | 1465 | CZ | TYR | C | 196 | 15116 | 13716 | 13831 | −53 | −2880 | 1711 | C |
| ATOM | 1466 | OH | TYR | C | 196 | 88.145 | 0.572 | 0.022 | 1.00 | 113.70 | | O |
| ANISOU | 1466 | OH | TYR | C | 196 | 14856 | 14205 | 14140 | 8 | −2724 | 1569 | O |
| ATOM | 1467 | N | LEU | C | 197 | 82.823 | −5.992 | 2.763 | 1.00 | 98.79 | | N |
| ANISOU | 1467 | N | LEU | C | 197 | 15916 | 10139 | 11481 | −484 | −3668 | 2535 | N |
| ATOM | 1468 | CA | LEU | C | 197 | 82.029 | −6.950 | 3.538 | 1.00 | 100.37 | | C |
| ANISOU | 1468 | CA | LEU | C | 197 | 16616 | 10011 | 11509 | −694 | −3838 | 2760 | C |
| ATOM | 1469 | C | LEU | C | 197 | 82.494 | −8.385 | 3.275 | 1.00 | 109.20 | | C |
| ANISOU | 1469 | C | LEU | C | 197 | 18068 | 10746 | 12678 | −369 | −4172 | 2775 | C |
| ATOM | 1470 | O | LEU | C | 197 | 82.622 | −9.164 | 4.219 | 1.00 | 112.34 | | O |
| ANISOU | 1470 | O | LEU | C | 197 | 18832 | 10907 | 12946 | −363 | −4460 | 2956 | O |
| ATOM | 1471 | CB | LEU | C | 197 | 80.521 | −6.799 | 3.294 | 1.00 | 97.91 | | C |
| ANISOU | 1471 | CB | LEU | C | 197 | 16429 | 9652 | 11120 | −1112 | −3580 | 2833 | C |
| ATOM | 1472 | CG | LEU | C | 197 | 79.617 | −7.459 | 4.322 | 1.00 | 104.14 | | C |
| ANISOU | 1472 | CG | LEU | C | 197 | 17662 | 10223 | 11683 | −1455 | −3689 | 3086 | C |
| ATOM | 1473 | CD1 | LEU | C | 197 | 79.477 | −6.607 | 5.567 | 1.00 | 103.81 | | C |
| ANISOU | 1473 | CD1 | LEU | C | 197 | 17534 | 10452 | 11455 | −1720 | −3622 | 3189 | C |
| ATOM | 1474 | CD2 | LEU | C | 197 | 78.267 | −7.733 | 3.747 | 1.00 | 105.12 | | C |
| ANISOU | 1474 | CD2 | LEU | C | 197 | 17943 | 10220 | 11776 | −1760 | −3502 | 3113 | C |
| ATOM | 1475 | N | HIS | C | 198 | 82.748 | −8.727 | 1.999 | 1.00 | 106.18 | | N |
| ANISOU | 1475 | N | HIS | C | 198 | 17567 | 10303 | 12474 | −92 | −4144 | 2583 | N |
| ATOM | 1476 | CA | HIS | C | 198 | 83.256 | −10.032 | 1.563 | 1.00 | 109.50 | | C |
| ANISOU | 1476 | CA | HIS | C | 198 | 18252 | 10377 | 12976 | 282 | −4458 | 2529 | C |
| ATOM | 1477 | C | HIS | C | 198 | 84.421 | −9.741 | 0.600 | 1.00 | 113.09 | | C |
| ANISOU | 1477 | C | HIS | C | 198 | 18270 | 11086 | 13613 | 728 | −4437 | 2244 | C |
| ATOM | 1478 | O | HIS | C | 198 | 84.211 | −9.784 | −0.611 | 1.00 | 112.16 | | O |
| ANISOU | 1478 | O | HIS | C | 198 | 18006 | 10997 | 13612 | 812 | −4274 | 2080 | O |
| ATOM | 1479 | CB | HIS | C | 198 | 82.147 | −10.847 | 0.862 | 1.00 | 110.41 | | C |
| ANISOU | 1479 | CB | HIS | C | 198 | 18694 | 10150 | 13106 | 115 | −4419 | 2563 | C |
| ATOM | 1480 | CG | HIS | C | 198 | 80.972 | −11.154 | 1.730 | 1.00 | 114.11 | | C |
| ANISOU | 1480 | CG | HIS | C | 198 | 19558 | 10411 | 13386 | −362 | −4411 | 2834 | C |
| ATOM | 1481 | CD2 | HIS | C | 198 | 80.848 | −12.064 | 2.725 | 1.00 | 119.34 | | C |
| ANISOU | 1481 | CD2 | HIS | C | 198 | 20690 | 10750 | 13903 | −463 | −4701 | 3075 | C |
| ATOM | 1482 | ND1 | HIS | C | 198 | 79.780 | −10.468 | 1.597 | 1.00 | 112.59 | | N |
| ANISOU | 1482 | ND1 | HIS | C | 198 | 19278 | 10372 | 13128 | −793 | −4074 | 2871 | N |
| ATOM | 1483 | CE1 | HIS | C | 198 | 78.970 | −10.981 | 2.509 | 1.00 | 113.65 | | C |
| ANISOU | 1483 | CE1 | HIS | C | 198 | 19804 | 10303 | 13076 | −1152 | −4156 | 3118 | C |
| ATOM | 1484 | NE2 | HIS | C | 198 | 79.570 | −11.942 | 3.215 | 1.00 | 118.00 | | N |
| ANISOU | 1484 | NE2 | HIS | C | 198 | 20708 | 10558 | 13568 | −986 | −4526 | 3264 | N |
| ATOM | 1485 | N | PRO | C | 199 | 85.627 | −9.362 | 1.086 | 1.00 | 109.97 | | N |
| ANISOU | 1485 | N | PRO | C | 199 | 17621 | 10930 | 13233 | 991 | −4569 | 2171 | N |
| ATOM | 1486 | CA | PRO | C | 199 | 86.702 | −9.002 | 0.144 | 1.00 | 109.67 | | C |
| ANISOU | 1486 | CA | PRO | C | 199 | 17115 | 11204 | 13352 | 1365 | −4508 | 1894 | C |
| ATOM | 1487 | C | PRO | C | 199 | 87.375 | −10.174 | −0.564 | 1.00 | 117.18 | | C |
| ANISOU | 1487 | C | PRO | C | 199 | 18166 | 11952 | 14404 | 1862 | −4786 | 1717 | C |
| ATOM | 1488 | O | PRO | C | 199 | 87.919 | −9.983 | −1.655 | 1.00 | 116.43 | | O |
| ANISOU | 1488 | O | PRO | C | 199 | 17708 | 12101 | 14429 | 2121 | −4672 | 1474 | O |
| ATOM | 1489 | CB | PRO | C | 199 | 87.684 | −8.217 | 1.013 | 1.00 | 111.82 | | C |
| ANISOU | 1489 | CB | PRO | C | 199 | 17092 | 11806 | 13587 | 1424 | −4561 | 1884 | C |
| ATOM | 1490 | CG | PRO | C | 199 | 87.504 | −8.776 | 2.373 | 1.00 | 118.43 | | C |
| ANISOU | 1490 | CG | PRO | C | 199 | 18354 | 12381 | 14265 | 1329 | −4842 | 2116 | C |
| ATOM | 1491 | CD | PRO | C | 199 | 86.069 | −9.217 | 2.489 | 1.00 | 113.01 | | C |
| ANISOU | 1491 | CD | PRO | C | 199 | 18104 | 11352 | 13482 | 950 | −4769 | 2326 | C |
| ATOM | 1492 | N | GLU | C | 200 | 87.336 | −11.375 | 0.063 | 1.00 | 117.19 | | N |
| ANISOU | 1492 | N | GLU | C | 200 | 18664 | 11515 | 14347 | 1995 | −5156 | 1837 | N |
| ATOM | 1493 | CA | GLU | C | 200 | 87.950 | −12.634 | −0.386 | 1.00 | 141.37 | | C |
| ANISOU | 1493 | CA | GLU | C | 200 | 21931 | 14290 | 17492 | 2490 | −5510 | 1688 | C |
| ATOM | 1494 | C | GLU | C | 200 | 87.450 | −13.133 | −1.749 | 1.00 | 141.41 | | C |
| ANISOU | 1494 | C | GLU | C | 200 | 21952 | 14162 | 17616 | 2585 | −5402 | 1513 | C |
| ATOM | 1495 | O | GLU | C | 200 | 87.726 | −12.530 | −2.785 | 1.00 | 88.68 | | O |
| ANISOU | 1495 | O | GLU | C | 200 | 14825 | 7841 | 11027 | 2685 | −5144 | 1295 | O |
| ATOM | 1496 | CB | GLU | C | 200 | 87.805 | −13.738 | 0.693 | 1.00 | 146.39 | | C |
| ANISOU | 1496 | CB | GLU | C | 200 | 23175 | 14426 | 18021 | 2513 | −5923 | 1920 | C |
| ATOM | 1497 | CG | GLU | C | 200 | 86.394 | −14.275 | 0.923 | 1.00 | 153.35 | | C |
| ANISOU | 1497 | CG | GLU | C | 200 | 24565 | 14885 | 18816 | 2062 | −5885 | 2179 | C |
| ATOM | 1498 | CD | GLU | C | 200 | 85.441 | −13.418 | 1.738 | 1.00 | 158.53 | | C |
| ANISOU | 1498 | CD | GLU | C | 200 | 25243 | 15679 | 19312 | 1477 | −5617 | 2436 | C |
| ATOM | 1499 | OE1 | GLU | C | 200 | 85.877 | −12.843 | 2.762 | 1.00 | 151.65 | | O |
| ANISOU | 1499 | OE1 | GLU | C | 200 | 24263 | 15024 | 18331 | 1419 | −5654 | 2539 | O |
| ATOM | 1500 | OE2 | GLU | C | 200 | 84.241 | −13.369 | 1.384 | 1.00 | 137.99 | | O |
| ANISOU | 1500 | OE2 | GLU | C | 200 | 22781 | 12965 | 16683 | 1083 | −5389 | 2525 | O |
| ATOM | 1501 | N | ALA | C | 201 | 84.168 | −12.692 | −1.323 | 1.00 | 120.81 | | N |
| ANISOU | 1501 | N | ALA | C | 201 | 19921 | 11178 | 14803 | 1317 | −4892 | 2023 | N |
| ATOM | 1502 | CA | ALA | C | 201 | 84.324 | −13.121 | −2.711 | 1.00 | 121.18 | | C |
| ANISOU | 1502 | CA | ALA | C | 201 | 19844 | 11198 | 15001 | 1634 | −4874 | 1756 | C |
| ATOM | 1503 | C | ALA | C | 201 | 84.321 | −11.961 | −3.730 | 1.00 | 121.12 | | C |
| ANISOU | 1503 | C | ALA | C | 201 | 19287 | 11670 | 15063 | 1594 | −4466 | 1572 | C |
| ATOM | 1504 | O | ALA | C | 201 | 84.082 | −12.192 | −4.920 | 1.00 | 119.66 | | O |
| ANISOU | 1504 | O | ALA | C | 201 | 19013 | 11486 | 14966 | 1721 | −4363 | 1387 | O |
| ATOM | 1505 | CB | ALA | C | 201 | 83.274 | −14.167 | −3.060 | 1.00 | 123.31 | | C |
| ANISOU | 1505 | CB | ALA | C | 201 | 20584 | 10992 | 15277 | 1482 | −4968 | 1817 | C |
| ATOM | 1506 | N | PHE | C | 202 | 84.635 | −10.724 | −3.266 | 1.00 | 116.05 | | N |
| ANISOU | 1506 | N | PHE | C | 202 | 18284 | 11431 | 14379 | 1430 | −4251 | 1618 | N |
| ATOM | 1507 | CA | PHE | C | 202 | 84.696 | −9.517 | −4.103 | 1.00 | 112.98 | | C |
| ANISOU | 1507 | CA | PHE | C | 202 | 17389 | 11492 | 14046 | 1361 | −3876 | 1484 | C |
| ATOM | 1508 | C | PHE | C | 202 | 85.820 | −9.588 | −5.136 | 1.00 | 118.55 | | C |
| ANISOU | 1508 | C | PHE | C | 202 | 17729 | 12446 | 14869 | 1831 | −3903 | 1200 | C |
| ATOM | 1509 | O | PHE | C | 202 | 85.537 | −9.579 | −6.336 | 1.00 | 117.68 | | O |
| ANISOU | 1509 | O | PHE | C | 202 | 17478 | 12416 | 14819 | 1901 | −3732 | 1047 | O |
| ATOM | 1510 | CB | PHE | C | 202 | 84.788 | −8.225 | −3.262 | 1.00 | 112.62 | | C |
| ANISOU | 1510 | CB | PHE | C | 202 | 17092 | 11765 | 13935 | 1072 | −3688 | 1605 | C |
| ATOM | 1511 | CG | PHE | C | 202 | 84.908 | −6.967 | −4.088 | 1.00 | 111.50 | | C |
| ANISOU | 1511 | CG | PHE | C | 202 | 16458 | 12052 | 13856 | 996 | −3330 | 1486 | C |
| ATOM | 1512 | CD1 | PHE | C | 202 | 83.784 | −6.381 | −4.660 | 1.00 | 111.77 | | C |
| ANISOU | 1512 | CD1 | PHE | C | 202 | 16461 | 12133 | 13874 | 671 | −3019 | 1524 | C |
| ATOM | 1513 | CD2 | PHE | C | 202 | 86.146 | −6.373 | −4.305 | 1.00 | 114.55 | | C |
| ANISOU | 1513 | CD2 | PHE | C | 202 | 16414 | 12800 | 14311 | 1243 | −3311 | 1337 | C |
| ATOM | 1514 | CE1 | PHE | C | 202 | 83.897 | −5.229 | −5.441 | 1.00 | 110.41 | | C |
| ANISOU | 1514 | CE1 | PHE | C | 202 | 15868 | 12327 | 13755 | 608 | −2708 | 1433 | C |
| ATOM | 1515 | CE2 | PHE | C | 202 | 86.259 | −5.225 | −5.092 | 1.00 | 115.16 | | C |
| ANISOU | 1515 | CE2 | PHE | C | 202 | 16061 | 13253 | 14440 | 1146 | −2985 | 1251 | C |
| ATOM | 1516 | CZ | PHE | C | 202 | 85.134 | −4.657 | −5.650 | 1.00 | 110.27 | | C |
| ANISOU | 1516 | CZ | PHE | C | 202 | 15451 | 12642 | 13806 | 832 | −2692 | 1309 | C |
| ATOM | 1517 | N | ALA | C | 203 | 87.087 | −9.656 | −4.675 | 1.00 | 116.94 | | N |
| ANISOU | 1517 | N | ALA | C | 203 | 17357 | 12392 | 14684 | 2155 | −4118 | 1120 | N |
| ATOM | 1518 | CA | ALA | C | 203 | 88.252 | −9.756 | −5.555 | 1.00 | 118.26 | | C |
| ANISOU | 1518 | CA | ALA | C | 203 | 17147 | 12846 | 14940 | 2620 | −4166 | 835 | C |
| ATOM | 1519 | C | ALA | C | 203 | 88.200 | −11.047 | −6.394 | 1.00 | 123.56 | | C |
| ANISOU | 1519 | C | ALA | C | 203 | 18055 | 13219 | 15672 | 2986 | −4387 | 659 | C |
| ATOM | 1520 | O | ALA | C | 203 | 88.771 | −11.087 | −7.484 | 1.00 | 123.54 | | O |
| ANISOU | 1520 | O | ALA | C | 203 | 17734 | 13470 | 15734 | 3312 | −4334 | 397 | O |
| ATOM | 1521 | CB | ALA | C | 203 | 89.530 | −9.695 | −4.738 | 1.00 | 121.52 | | C |
| ANISOU | 1521 | CB | ALA | C | 203 | 17382 | 13447 | 15344 | 2878 | −4393 | 792 | C |
| ATOM | 1522 | N | SER | C | 204 | 87.466 | −12.074 | −5.901 | 1.00 | 121.20 | | N |
| ANISOU | 1522 | N | SER | C | 204 | 18317 | 12389 | 15346 | 2905 | −4623 | 803 | N |
| ATOM | 1523 | CA | SER | C | 204 | 87.245 | −13.355 | −6.572 | 1.00 | 123.75 | | C |
| ANISOU | 1523 | CA | SER | C | 204 | 18959 | 12329 | 15731 | 3188 | −4860 | 665 | C |
| ATOM | 1524 | C | SER | C | 204 | 86.437 | −13.163 | −7.855 | 1.00 | 125.10 | | C |
| ANISOU | 1524 | C | SER | C | 204 | 19000 | 12589 | 15942 | 3069 | −4559 | 541 | C |
| ATOM | 1525 | O | SER | C | 204 | 86.758 | −13.781 | −8.873 | 1.00 | 126.67 | | O |
| ANISOU | 1525 | O | SER | C | 204 | 19110 | 12804 | 16213 | 3446 | −4639 | 274 | O |
| ATOM | 1526 | CB | SER | C | 204 | 86.531 | −14.334 | −5.644 | 1.00 | 129.55 | | C |
| ANISOU | 1526 | CB | SER | C | 204 | 20337 | 12473 | 16414 | 3014 | −5150 | 905 | C |
| ATOM | 1527 | OG | SER | C | 204 | 87.441 | −15.085 | −4.857 | 1.00 | 143.32 | | O |
| ANISOU | 1527 | OG | SER | C | 204 | 22304 | 13990 | 18160 | 3397 | −5587 | 892 | O |
| ATOM | 1528 | N | LEU | C | 205 | 85.399 | −12.303 | −7.814 | 1.00 | 117.50 | | N |
| ANISOU | 1528 | N | LEU | C | 205 | 18012 | 11708 | 14924 | 2566 | −4222 | 716 | N |
| ATOM | 1529 | CA | LEU | C | 205 | 84.571 | −12.024 | −8.991 | 1.00 | 114.87 | | C |
| ANISOU | 1529 | CA | LEU | C | 205 | 17551 | 11483 | 14612 | 2428 | −3926 | 615 | C |
| ATOM | 1530 | C | LEU | C | 205 | 85.164 | −10.905 | −9.839 | 1.00 | 117.44 | | C |
| ANISOU | 1530 | C | LEU | C | 205 | 17289 | 12380 | 14954 | 2511 | −3609 | 464 | C |
| ATOM | 1531 | O | LEU | C | 205 | 85.173 | −11.032 | −11.060 | 1.00 | 117.01 | | O |
| ANISOU | 1531 | O | LEU | C | 205 | 17050 | 12473 | 14934 | 2705 | −3500 | 252 | O |
| ATOM | 1532 | CB | LEU | C | 205 | 83.098 | −11.737 | −8.616 | 1.00 | 111.80 | | C |
| ANISOU | 1532 | CB | LEU | C | 205 | 17423 | 10907 | 14149 | 1877 | −3733 | 849 | C |
| ATOM | 1533 | CG | LEU | C | 205 | 82.054 | −11.646 | −9.757 | 1.00 | 113.49 | | C |
| ANISOU | 1533 | CG | LEU | C | 205 | 17594 | 11154 | 14373 | 1721 | −3477 | 755 | C |
| ATOM | 1534 | CD1 | LEU | C | 205 | 81.929 | −12.959 | −10.532 | 1.00 | 116.06 | | C |
| ANISOU | 1534 | CD1 | LEU | C | 205 | 18199 | 11129 | 14767 | 1995 | −3713 | 573 | C |
| ATOM | 1535 | CD2 | LEU | C | 205 | 80.695 | −11.266 | −9.208 | 1.00 | 112.76 | | C |
| ANISOU | 1535 | CD2 | LEU | C | 205 | 17694 | 10960 | 14191 | 1177 | −3284 | 983 | C |
| ATOM | 1536 | N | SER | C | 206 | 85.678 | −9.830 | −9.195 | 1.00 | 112.95 | | N |
| ANISOU | 1536 | N | SER | C | 206 | 16433 | 12129 | 14353 | 2359 | −3471 | 573 | N |
| ATOM | 1537 | CA | SER | C | 206 | 86.295 | −8.671 | −9.855 | 1.00 | 111.39 | | C |
| ANISOU | 1537 | CA | SER | C | 206 | 15687 | 12468 | 14167 | 2378 | −3175 | 472 | C |
| ATOM | 1538 | C | SER | C | 206 | 87.390 | −9.089 | −10.859 | 1.00 | 119.56 | | C |
| ANISOU | 1538 | C | SER | C | 206 | 16414 | 13762 | 15253 | 2880 | −3256 | 169 | C |
| ATOM | 1539 | O | SER | C | 206 | 87.376 | −8.636 | −12.011 | 1.00 | 117.93 | | O |
| ANISOU | 1539 | O | SER | C | 206 | 15901 | 13862 | 15046 | 2917 | −3004 | 36 | O |
| ATOM | 1540 | CB | SER | C | 206 | 86.853 | −7.697 | −8.820 | 1.00 | 113.18 | | C |
| ANISOU | 1540 | CB | SER | C | 206 | 15717 | 12917 | 14367 | 2201 | −3130 | 613 | C |
| ATOM | 1541 | OG | SER | C | 206 | 85.844 | −7.238 | −7.935 | 1.00 | 116.99 | | O |
| ANISOU | 1541 | OG | SER | C | 206 | 16438 | 13224 | 14788 | 1746 | −3032 | 865 | O |
| ATOM | 1542 | N | ALA | C | 207 | 88.297 | −9.990 | −10.427 | 1.00 | 121.00 | | N |
| ANISOU | 1542 | N | ALA | C | 207 | 16693 | 13817 | 15465 | 3277 | −3619 | 55 | N |
| ATOM | 1543 | CA | ALA | C | 207 | 89.383 | −10.523 | −11.249 | 1.00 | 124.54 | | C |
| ANISOU | 1543 | CA | ALA | C | 207 | 16870 | 14499 | 15952 | 3806 | −3756 | −262 | C |
| ATOM | 1544 | C | ALA | C | 207 | 88.857 | −11.456 | −12.345 | 1.00 | 130.42 | | C |
| ANISOU | 1544 | C | ALA | C | 207 | 17793 | 15038 | 16724 | 4019 | −3809 | −452 | C |
| ATOM | 1545 | O | ALA | C | 207 | 89.446 | −11.514 | −13.428 | 1.00 | 131.25 | | O |
| ANISOU | 1545 | O | ALA | C | 207 | 17557 | 15480 | 16834 | 4336 | −3737 | −721 | O |
| ATOM | 1546 | CB | ALA | C | 207 | 90.387 | −11.255 | −10.375 | 1.00 | 129.11 | | C |
| ANISOU | 1546 | CB | ALA | C | 207 | 17560 | 14947 | 16549 | 4170 | −4164 | −324 | C |
| ATOM | 1547 | N | ARG | C | 208 | 87.753 | −12.182 | −12.058 | 1.00 | 127.31 | | N |
| ANISOU | 1547 | N | ARG | C | 208 | 17923 | 14110 | 16340 | 3831 | −3932 | −318 | N |
| ATOM | 1548 | CA | ARG | C | 208 | 87.103 | −13.102 | −12.996 | 1.00 | 128.56 | | C |
| ANISOU | 1548 | CA | ARG | C | 208 | 18314 | 14001 | 16532 | 3970 | −4001 | −480 | C |
| ATOM | 1549 | C | ARG | C | 208 | 86.373 | −12.322 | −14.100 | 1.00 | 130.09 | | C |
| ANISOU | 1549 | C | ARG | C | 208 | 18245 | 14496 | 16688 | 3736 | −3590 | −513 | C |
| ATOM | 1550 | O | ARG | C | 208 | 86.287 | −12.799 | −15.236 | 1.00 | 130.64 | | O |
| ANISOU | 1550 | O | ARG | C | 208 | 18260 | 14605 | 16770 | 3972 | −3577 | −750 | O |
| ATOM | 1551 | CB | ARG | C | 208 | 86.137 | −14.034 | −12.253 | 1.00 | 129.46 | | C |
| ANISOU | 1551 | CB | ARG | C | 208 | 19061 | 13466 | 16660 | 3761 | −4243 | −293 | C |
| ATOM | 1552 | N | ILE | C | 209 | 85.861 | −11.116 | −13.756 | 1.00 | 123.43 | | N |
| ANISOU | 1552 | N | ILE | C | 209 | 17241 | 13864 | 15792 | 3290 | −3268 | −284 | N |
| ATOM | 1553 | CA | ILE | C | 209 | 85.163 | −10.188 | −14.653 | 1.00 | 120.04 | | C |
| ANISOU | 1553 | CA | ILE | C | 209 | 16561 | 13734 | 15316 | 3033 | −2871 | −268 | C |
| ATOM | 1554 | C | ILE | C | 209 | 86.173 | −9.576 | −15.633 | 1.00 | 126.20 | | C |
| ANISOU | 1554 | C | ILE | C | 209 | 16790 | 15085 | 16074 | 3304 | −2694 | −472 | C |
| ATOM | 1555 | O | ILE | C | 209 | 85.897 | −9.519 | −16.836 | 1.00 | 125.38 | | O |
| ANISOU | 1555 | O | ILE | C | 209 | 16521 | 15180 | 15938 | 3388 | −2520 | −624 | O |
| ATOM | 1556 | CB | ILE | C | 209 | 84.392 | −9.113 | −13.822 | 1.00 | 118.96 | | C |
| ANISOU | 1556 | CB | ILE | C | 209 | 16463 | 13603 | 15134 | 2500 | −2640 | 39 | C |
| ATOM | 1557 | CG1 | ILE | C | 209 | 83.162 | −9.711 | −13.076 | 1.00 | 118.30 | | C |
| ANISOU | 1557 | CG1 | ILE | C | 209 | 16901 | 13009 | 15037 | 2186 | −2763 | 229 | C |
| ATOM | 1558 | CG2 | ILE | C | 209 | 84.010 | −7.873 | −14.643 | 1.00 | 116.54 | | C |
| ANISOU | 1558 | CG2 | ILE | C | 209 | 15821 | 13682 | 14779 | 2272 | −2234 | 64 | C |
| ATOM | 1559 | CD1 | ILE | C | 209 | 82.104 | −10.481 | −13.892 | 1.00 | 124.00 | | C |
| ANISOU | 1559 | CD1 | ILE | C | 209 | 17917 | 13431 | 15768 | 2183 | −2807 | 130 | C |
| ATOM | 1560 | N | GLN | C | 210 | 87.350 | −9.146 | −15.105 | 1.00 | 125.10 | | N |
| ANISOU | 1560 | N | GLN | C | 210 | 16367 | 15222 | 15943 | 3435 | −2748 | −479 | N |
| ATOM | 1561 | CA | GLN | C | 210 | 88.472 | −8.545 | −15.841 | 1.00 | 126.34 | | C |
| ANISOU | 1561 | CA | GLN | C | 210 | 15974 | 15958 | 16070 | 3662 | −2598 | −655 | C |
| ATOM | 1562 | C | GLN | C | 210 | 89.091 | −9.463 | −16.917 | 1.00 | 134.00 | | C |
| ANISOU | 1562 | C | GLN | C | 210 | 16802 | 17079 | 17035 | 4179 | −2731 | −1008 | C |
| ATOM | 1563 | O | GLN | C | 210 | 89.671 | −8.954 | −17.882 | 1.00 | 134.29 | | O |
| ANISOU | 1563 | O | GLN | C | 210 | 16387 | 17626 | 17010 | 4301 | −2517 | −1157 | O |
| ATOM | 1564 | CB | GLN | C | 210 | 89.553 | −8.048 | −14.867 | 1.00 | 128.70 | | C |
| ANISOU | 1564 | CB | GLN | C | 210 | 16058 | 16455 | 16386 | 3692 | −2692 | −599 | C |
| ATOM | 1565 | N | ALA | C | 211 | 88.975 | −10.804 | −16.747 | 1.00 | 132.87 | | N |
| ANISOU | 1565 | N | ALA | C | 211 | 17041 | 16496 | 16947 | 4479 | −3090 | −1141 | N |
| ATOM | 1566 | CA | ALA | C | 211 | 89.485 | −11.807 | −17.691 | 1.00 | 135.98 | | C |
| ANISOU | 1566 | CA | ALA | C | 211 | 17366 | 16953 | 17347 | 5003 | −3275 | −1503 | C |
| ATOM | 1567 | C | ALA | C | 211 | 88.627 | −11.838 | −18.962 | 1.00 | 137.53 | | C |
| ANISOU | 1567 | C | ALA | C | 211 | 17543 | 17216 | 17495 | 4942 | −3051 | −1604 | C |
| ATOM | 1568 | O | ALA | C | 211 | 89.154 | −11.995 | −20.068 | 1.00 | 138.86 | | O |
| ANISOU | 1568 | O | ALA | C | 211 | 17383 | 17769 | 17609 | 5271 | −2986 | −1888 | O |
| ATOM | 1569 | CB | ALA | C | 211 | 89.510 | −13.182 | −17.036 | 1.00 | 139.93 | | C |
| ANISOU | 1569 | CB | ALA | C | 211 | 18351 | 16882 | 17933 | 5293 | −3744 | −1580 | C |
| ATOM | 1570 | N | THR | C | 212 | 87.307 | −11.668 | −18.795 | 1.00 | 130.32 | | N |
| ANISOU | 1570 | N | THR | C | 212 | 16962 | 15966 | 16589 | 4520 | −2929 | −1379 | N |
| ATOM | 1571 | CA | THR | C | 212 | 86.344 | −11.643 | −19.895 | 1.00 | 128.29 | | C |
| ANISOU | 1571 | CA | THR | C | 212 | 16722 | 15742 | 16282 | 4407 | −2716 | −1443 | C |
| ATOM | 1572 | C | THR | C | 212 | 86.114 | −10.218 | −20.416 | 1.00 | 128.25 | | C |
| ANISOU | 1572 | C | THR | C | 212 | 16340 | 16208 | 16180 | 4076 | −2273 | −1291 | C |
| ATOM | 1573 | O | THR | C | 212 | 85.378 | −10.058 | −21.385 | 1.00 | 126.16 | | O |
| ANISOU | 1573 | O | THR | C | 212 | 16028 | 16056 | 15852 | 3998 | −2070 | −1344 | O |
| ATOM | 1574 | CB | THR | C | 212 | 85.028 | −12.321 | −19.477 | 1.00 | 134.78 | | C |
| ANISOU | 1574 | CB | THR | C | 212 | 18099 | 15956 | 17156 | 4144 | −2844 | −1311 | C |
| ATOM | 1575 | CG2 | THR | C | 212 | 85.141 | −13.834 | −19.417 | 1.00 | 137.23 | | C |
| ANISOU | 1575 | CG2 | THR | C | 212 | 18787 | 15802 | 17554 | 4504 | −3266 | −1518 | C |
| ATOM | 1576 | OG1 | THR | C | 212 | 84.601 | −11.805 | −18.213 | 1.00 | 132.24 | | O |
| ANISOU | 1576 | OG1 | THR | C | 212 | 17981 | 15415 | 16851 | 3725 | −2819 | −975 | O |
| ATOM | 1577 | N | GLN | C | 213 | 86.753 | −9.193 | −19.787 | 1.00 | 123.83 | | N |
| ANISOU | 1577 | N | GLN | C | 213 | 15524 | 15917 | 15610 | 3891 | −2139 | −1110 | N |
| ATOM | 1578 | CA | GLN | C | 213 | 86.620 | −7.762 | −20.119 | 1.00 | 120.78 | | C |
| ANISOU | 1578 | CA | GLN | C | 213 | 14811 | 15932 | 15149 | 3550 | −1749 | −931 | C |
| ATOM | 1579 | C | GLN | C | 213 | 86.962 | −7.413 | −21.573 | 1.00 | 124.84 | | C |
| ANISOU | 1579 | C | GLN | C | 213 | 14908 | 16974 | 15553 | 3719 | −1505 | −1109 | C |
| ATOM | 1580 | O | GLN | C | 213 | 86.133 | −6.800 | −22.249 | 1.00 | 122.22 | | O |
| ANISOU | 1580 | O | GLN | C | 213 | 14537 | 16752 | 15150 | 3468 | −1232 | −1006 | O |
| ATOM | 1581 | CB | GLN | C | 213 | 87.422 | −6.879 | −19.149 | 1.00 | 121.68 | | C |
| ANISOU | 1581 | CB | GLN | C | 213 | 14724 | 16221 | 15289 | 3386 | −1716 | −757 | C |
| ATOM | 1582 | N | GLU | C | 214 | 88.167 | −7.794 | −22.050 | 1.00 | 124.02 | | N |
| ANISOU | 1582 | N | GLU | C | 214 | 14489 | 17213 | 15419 | 4144 | −1605 | −1378 | N |
| ATOM | 1583 | CA | GLU | C | 214 | 88.600 | −7.544 | −23.432 | 1.00 | 124.67 | | C |
| ANISOU | 1583 | CA | GLU | C | 214 | 14151 | 17848 | 15368 | 4332 | −1385 | −1570 | C |
| ATOM | 1584 | C | GLU | C | 214 | 87.795 | −8.371 | −24.439 | 1.00 | 127.63 | | C |
| ANISOU | 1584 | C | GLU | C | 214 | 14711 | 18080 | 15701 | 4522 | −1422 | −1772 | C |
| ATOM | 1585 | O | GLU | C | 214 | 87.531 | −7.900 | −25.544 | 1.00 | 126.36 | | O |
| ANISOU | 1585 | O | GLU | C | 214 | 14325 | 18274 | 15412 | 4488 | −1159 | −1813 | O |
| ATOM | 1586 | CB | GLU | C | 214 | 90.125 | −7.745 | −23.618 | 1.00 | 129.87 | | C |
| ANISOU | 1586 | CB | GLU | C | 214 | 14378 | 18976 | 15990 | 4726 | −1476 | −1816 | C |
| ATOM | 1587 | CG | GLU | C | 214 | 90.716 | −9.037 | −23.061 | 1.00 | 143.03 | | C |
| ANISOU | 1587 | CG | GLU | C | 214 | 16225 | 20368 | 17753 | 5175 | −1905 | −2058 | C |
| ATOM | 1588 | CD | GLU | C | 214 | 90.438 | −10.311 | −23.838 | 1.00 | 162.78 | | C |
| ANISOU | 1588 | CD | GLU | C | 214 | 18923 | 22671 | 20255 | 5595 | −2127 | −2380 | C |
| ATOM | 1589 | OE1 | GLU | C | 214 | 89.924 | −11.273 | −23.222 | 1.00 | 151.39 | | O |
| ANISOU | 1589 | OE1 | GLU | C | 214 | 17967 | 20625 | 18930 | 5665 | −2433 | −2383 | O |
| ATOM | 1590 | OE2 | GLU | C | 214 | 90.694 | −10.337 | −25.065 | 1.00 | 158.66 | | O |
| ANISOU | 1590 | OE2 | GLU | C | 214 | 18082 | 22590 | 19610 | 5833 | −1992 | −2622 | O |
| ATOM | 1591 | N | ALA | C | 215 | 87.395 | −9.592 | −24.031 | 1.00 | 124.73 | | N |
| ANISOU | 1591 | N | ALA | C | 215 | 14769 | 17184 | 15439 | 4706 | −1754 | −1889 | N |
| ATOM | 1592 | CA | ALA | C | 215 | 86.625 | −10.548 | −24.825 | 1.00 | 125.22 | | C |
| ANISOU | 1592 | CA | ALA | C | 215 | 15073 | 17011 | 15494 | 4892 | −1860 | −2104 | C |
| ATOM | 1593 | C | ALA | C | 215 | 85.174 | −10.109 | −25.025 | 1.00 | 124.46 | | C |
| ANISOU | 1593 | C | ALA | C | 215 | 15206 | 16712 | 15370 | 4470 | −1648 | −1899 | C |
| ATOM | 1594 | O | ALA | C | 215 | 84.629 | −10.310 | −26.109 | 1.00 | 124.19 | | O |
| ANISOU | 1594 | O | ALA | C | 215 | 15140 | 16792 | 15255 | 4558 | −1549 | −2057 | O |
| ATOM | 1595 | CB | ALA | C | 215 | 86.677 | −11.925 | −24.179 | 1.00 | 128.64 | | C |
| ANISOU | 1595 | CB | ALA | C | 215 | 15922 | 16890 | 16063 | 5167 | −2301 | −2253 | C |
| ATOM | 1596 | N | ARG | C | 216 | 84.549 | −9.522 | −23.985 | 1.00 | 116.86 | | N |
| ANISOU | 1596 | N | ARG | C | 216 | 14464 | 15472 | 14466 | 4031 | −1587 | −1568 | N |
| ATOM | 1597 | CA | ARG | C | 216 | 83.173 | −9.039 | −24.057 | 1.00 | 112.65 | | C |
| ANISOU | 1597 | CA | ARG | C | 216 | 14135 | 14763 | 13905 | 3621 | −1394 | −1371 | C |
| ATOM | 1598 | C | ARG | C | 216 | 83.098 | −7.674 | −24.710 | 1.00 | 113.07 | | C |
| ANISOU | 1598 | C | ARG | C | 216 | 13838 | 15288 | 13835 | 3389 | −1006 | −1221 | C |
| ATOM | 1599 | O | ARG | C | 216 | 82.156 | −7.442 | −25.461 | 1.00 | 111.29 | | O |
| ANISOU | 1599 | O | ARG | C | 216 | 13664 | 15085 | 13537 | 3234 | −833 | −1196 | O |
| ATOM | 1600 | CB | ARG | C | 216 | 82.480 | −9.056 | −22.690 | 1.00 | 110.63 | | C |
| ANISOU | 1600 | CB | ARG | C | 216 | 14280 | 14007 | 13748 | 3272 | −1517 | −1114 | C |
| ATOM | 1601 | CG | ARG | C | 216 | 81.695 | −10.343 | −22.447 | 1.00 | 119.37 | | C |
| ANISOU | 1601 | CG | ARG | C | 216 | 15857 | 14564 | 14934 | 3325 | −1806 | −1214 | C |
| ATOM | 1602 | CD | ARG | C | 216 | 81.870 | −10.892 | −21.042 | 1.00 | 124.35 | | C |
| ANISOU | 1602 | CD | ARG | C | 216 | 16827 | 14749 | 15670 | 3245 | −2090 | −1068 | C |
| ATOM | 1603 | NE | ARG | C | 216 | 81.114 | −10.133 | −20.043 | 1.00 | 123.98 | | N |
| ANISOU | 1603 | NE | ARG | C | 216 | 16952 | 14537 | 15618 | 2749 | −1963 | −747 | N |
| ATOM | 1604 | CZ | ARG | C | 216 | 81.661 | −9.459 | −19.034 | 1.00 | 133.17 | | C |
| ANISOU | 1604 | CZ | ARG | C | 216 | 18055 | 15746 | 16796 | 2579 | −1945 | −533 | C |
| ATOM | 1605 | NH1 | ARG | C | 216 | 82.978 | −9.446 | −18.869 | 1.00 | 116.27 | | N |
| ANISOU | 1605 | NH1 | ARG | C | 216 | 15685 | 13810 | 14682 | 2851 | −2046 | −597 | N |
| ATOM | 1606 | NH2 | ARG | C | 216 | 80.893 | −8.802 | −18.178 | 1.00 | 119.45 | | N |
| ANISOU | 1606 | NH2 | ARG | C | 216 | 16475 | 13869 | 15040 | 2143 | −1831 | −274 | N |
| ATOM | 1607 | N | GLU | C | 217 | 84.113 | −6.799 | −24.483 | 1.00 | 108.69 | | N |
| ANISOU | 1607 | N | GLU | C | 217 | 12928 | 15114 | 13254 | 3375 | −880 | −1130 | N |
| ATOM | 1608 | CA | GLU | C | 217 | 84.206 | −5.455 | −25.075 | 1.00 | 106.45 | | C |
| ANISOU | 1608 | CA | GLU | C | 217 | 12307 | 15282 | 12857 | 3149 | −525 | −969 | C |
| ATOM | 1609 | C | GLU | C | 217 | 84.128 | −5.439 | −26.623 | 1.00 | 110.62 | | C |
| ANISOU | 1609 | C | GLU | C | 217 | 12592 | 16213 | 13226 | 3335 | −340 | −1144 | C |
| ATOM | 1610 | O | GLU | C | 217 | 83.702 | −4.429 | −27.183 | 1.00 | 108.12 | | O |
| ANISOU | 1610 | O | GLU | C | 217 | 12155 | 16119 | 12807 | 3092 | −60 | −982 | O |
| ATOM | 1611 | CB | GLU | C | 217 | 85.460 | −4.715 | −24.589 | 1.00 | 108.55 | | C |
| ANISOU | 1611 | CB | GLU | C | 217 | 12239 | 15871 | 13135 | 3119 | −466 | −875 | C |
| ATOM | 1612 | N | ARG | C | 218 | 84.527 | −6.549 | −27.302 | 1.00 | 109.54 | | N |
| ANISOU | 1612 | N | ARG | C | 218 | 12394 | 16164 | 13060 | 3773 | −509 | −1479 | N |
| ATOM | 1613 | CA | ARG | C | 218 | 84.468 | −6.677 | −28.765 | 1.00 | 110.20 | | C |
| ANISOU | 1613 | CA | ARG | C | 218 | 12253 | 16639 | 12978 | 3994 | −364 | −1689 | C |
| ATOM | 1614 | C | ARG | C | 218 | 83.056 | −7.026 | −29.232 | 1.00 | 112.46 | | C |
| ANISOU | 1614 | C | ARG | C | 218 | 12850 | 16635 | 13245 | 3887 | −349 | −1709 | C |
| ATOM | 1615 | O | ARG | C | 218 | 82.604 | −6.512 | −30.257 | 1.00 | 111.32 | | O |
| ANISOU | 1615 | O | ARG | C | 218 | 12555 | 16794 | 12945 | 3832 | −113 | −1700 | O |
| ATOM | 1616 | CB | ARG | C | 218 | 85.519 | −7.674 | −29.312 | 1.00 | 113.21 | | C |
| ANISOU | 1616 | CB | ARG | C | 218 | 12428 | 17258 | 13327 | 4523 | −556 | −2072 | C |
| ATOM | 1617 | CG | ARG | C | 218 | 85.232 | −9.171 | −29.106 | 1.00 | 120.14 | | C |
| ANISOU | 1617 | CG | ARG | C | 218 | 13672 | 17645 | 14330 | 4830 | −930 | −2338 | C |
| ATOM | 1618 | CD | ARG | C | 218 | 86.287 | −10.062 | −29.741 | 1.00 | 128.92 | | C |
| ANISOU | 1618 | CD | ARG | C | 218 | 14549 | 19037 | 15400 | 5386 | −1115 | −2741 | C |
| ATOM | 1619 | NE | ARG | C | 218 | 87.597 | −9.915 | −29.097 | 1.00 | 136.68 | | N |
| ANISOU | 1619 | NE | ARG | C | 218 | 15289 | 20218 | 16425 | 5531 | −1210 | −2752 | N |
| ATOM | 1620 | CZ | ARG | C | 218 | 88.116 | −10.778 | −28.227 | 1.00 | 149.69 | | C |
| ANISOU | 1620 | CZ | ARG | C | 218 | 17141 | 21513 | 18223 | 5775 | −1567 | −2870 | C |
| ATOM | 1621 | NH1 | ARG | C | 218 | 89.308 | −10.553 | −27.689 | 1.00 | 134.97 | | N |
| ANISOU | 1621 | NH1 | ARG | C | 218 | 15015 | 19890 | 16379 | 5904 | −1634 | −2882 | N |
| ATOM | 1622 | NH2 | ARG | C | 218 | 87.451 | −11.878 | −27.897 | 1.00 | 137.45 | | N |
| ANISOU | 1622 | NH2 | ARG | C | 218 | 16061 | 19367 | 16798 | 5891 | −1869 | −2978 | N |
| ATOM | 1623 | N | LEU | C | 219 | 82.366 | −7.892 | −28.466 | 1.00 | 108.62 | | N |
| ANISOU | 1623 | N | LEU | C | 219 | 12796 | 15569 | 12906 | 3843 | −604 | −1729 | N |
| ATOM | 1624 | CA | LEU | C | 219 | 80.995 | −8.336 | −28.726 | 1.00 | 107.02 | | C |
| ANISOU | 1624 | CA | LEU | C | 219 | 12920 | 15032 | 12709 | 3708 | −630 | −1755 | C |
| ATOM | 1625 | C | LEU | C | 219 | 80.035 | −7.197 | −28.378 | 1.00 | 106.73 | | C |
| ANISOU | 1625 | C | LEU | C | 219 | 12950 | 14958 | 12644 | 3234 | −386 | −1424 | C |
| ATOM | 1626 | O | LEU | C | 219 | 79.012 | −7.013 | −29.039 | 1.00 | 104.98 | | O |
| ANISOU | 1626 | O | LEU | C | 219 | 12775 | 14782 | 12332 | 3116 | −242 | −1422 | O |
| ATOM | 1627 | CB | LEU | C | 219 | 80.669 | −9.584 | −27.886 | 1.00 | 108.11 | | C |
| ANISOU | 1627 | CB | LEU | C | 219 | 13498 | 14561 | 13017 | 3760 | −985 | −1844 | C |
| ATOM | 1628 | CG | LEU | C | 219 | 81.799 | −10.595 | −27.680 | 1.00 | 116.63 | | C |
| ANISOU | 1628 | CG | LEU | C | 219 | 14578 | 15557 | 14179 | 4198 | −1298 | −2099 | C |
| ATOM | 1629 | CD1 | LEU | C | 219 | 81.525 | −11.479 | −26.490 | 1.00 | 117.26 | | C |
| ANISOU | 1629 | CD1 | LEU | C | 219 | 15118 | 15005 | 14432 | 4126 | −1623 | −2041 | C |
| ATOM | 1630 | CD2 | LEU | C | 219 | 82.062 | −11.416 | −28.933 | 1.00 | 122.53 | | C |
| ANISOU | 1630 | CD2 | LEU | C | 219 | 15206 | 16497 | 14852 | 4633 | −1378 | −2491 | C |
| ATOM | 1631 | N | ILE | C | 220 | 80.394 | −6.410 | −27.360 | 1.00 | 101.57 | | N |
| ANISOU | 1631 | N | ILE | C | 220 | 12284 | 14247 | 12060 | 2985 | −346 | −1161 | N |
| ATOM | 1632 | CA | ILE | C | 220 | 79.618 | −5.262 | −26.927 | 1.00 | 98.23 | | C |
| ANISOU | 1632 | CA | ILE | C | 220 | 11911 | 13791 | 11620 | 2559 | −137 | −858 | C |
| ATOM | 1633 | C | ILE | C | 220 | 79.740 | −4.140 | −27.978 | 1.00 | 100.56 | | C |
| ANISOU | 1633 | C | ILE | C | 220 | 11866 | 14589 | 11753 | 2516 | 179 | −780 | C |
| ATOM | 1634 | O | ILE | C | 220 | 78.714 | −3.640 | −28.425 | 1.00 | 97.80 | | O |
| ANISOU | 1634 | O | ILE | C | 220 | 11586 | 14248 | 11326 | 2328 | 336 | −693 | O |
| ATOM | 1635 | CB | ILE | C | 220 | 79.977 | −4.868 | −25.447 | 1.00 | 100.68 | | C |
| ANISOU | 1635 | CB | ILE | C | 220 | 12320 | 13876 | 12056 | 2329 | −219 | −630 | C |
| ATOM | 1636 | CG1 | ILE | C | 220 | 78.711 | −4.846 | −24.527 | 1.00 | 98.80 | | C |
| ANISOU | 1636 | CG1 | ILE | C | 220 | 12447 | 13217 | 11878 | 1966 | −252 | −456 | C |
| ATOM | 1637 | CG2 | ILE | C | 220 | 80.831 | −3.591 | −25.278 | 1.00 | 101.06 | | C |
| ANISOU | 1637 | CG2 | ILE | C | 220 | 12037 | 14286 | 12075 | 2210 | −25 | −446 | C |
| ATOM | 1638 | CD1 | ILE | C | 220 | 77.404 | −4.093 | −25.044 | 1.00 | 105.70 | | C |
| ANISOU | 1638 | CD1 | ILE | C | 220 | 13340 | 14167 | 12652 | 1694 | −9 | −341 | C |
| ATOM | 1639 | N | GLN | C | 221 | 80.982 | −3.839 | −28.442 | 1.00 | 99.00 | | N |
| ANISOU | 1639 | N | GLN | C | 221 | 11305 | 14822 | 11489 | 2712 | 258 | −835 | N |
| ATOM | 1640 | CA | GLN | C | 221 | 81.292 | −2.804 | −29.439 | 1.00 | 98.53 | | C |
| ANISOU | 1640 | CA | GLN | C | 221 | 10904 | 15274 | 11259 | 2669 | 552 | −746 | C |
| ATOM | 1641 | C | GLN | C | 221 | 80.744 | −3.090 | −30.819 | 1.00 | 101.96 | | C |
| ANISOU | 1641 | C | GLN | C | 221 | 11280 | 15935 | 11525 | 2848 | 649 | −921 | C |
| ATOM | 1642 | O | GLN | C | 221 | 80.329 | −2.146 | −31.491 | 1.00 | 99.97 | | O |
| ANISOU | 1642 | O | GLN | C | 221 | 10920 | 15929 | 11136 | 2685 | 891 | −765 | O |
| ATOM | 1643 | CB | GLN | C | 221 | 82.799 | −2.518 | −29.510 | 1.00 | 102.13 | | C |
| ANISOU | 1643 | CB | GLN | C | 221 | 10977 | 16146 | 11682 | 2808 | 599 | −769 | C |
| ATOM | 1644 | CG | GLN | C | 221 | 83.164 | −1.066 | −29.212 | 1.00 | 109.32 | | C |
| ANISOU | 1644 | CG | GLN | C | 221 | 11701 | 17264 | 12572 | 2466 | 827 | −453 | C |
| ATOM | 1645 | CD | GLN | C | 221 | 82.516 | −0.509 | −27.960 | 1.00 | 118.33 | | C |
| ANISOU | 1645 | CD | GLN | C | 221 | 13121 | 17976 | 13864 | 2112 | 784 | −205 | C |
| ATOM | 1646 | NE2 | GLN | C | 221 | 81.834 | 0.612 | −28.119 | 1.00 | 108.97 | | N |
| ANISOU | 1646 | NE2 | GLN | C | 221 | 11963 | 16813 | 12627 | 1804 | 989 | 37 | N |
| ATOM | 1647 | OE1 | GLN | C | 221 | 82.612 | −1.068 | −26.855 | 1.00 | 109.88 | | O |
| ANISOU | 1647 | OE1 | GLN | C | 221 | 12243 | 16562 | 12945 | 2119 | 563 | −230 | O |
| ATOM | 1648 | N | LYS | C | 222 | 80.735 | −4.374 | −31.246 | 1.00 | 100.47 | | N |
| ANISOU | 1648 | N | LYS | C | 222 | 11175 | 15654 | 11344 | 3186 | 450 | −1244 | N |
| ATOM | 1649 | CA | LYS | C | 222 | 80.174 | −4.778 | −32.542 | 1.00 | 101.40 | | C |
| ANISOU | 1649 | CA | LYS | C | 222 | 11255 | 15966 | 11305 | 3379 | 512 | −1453 | C |
| ATOM | 1650 | C | LYS | C | 222 | 78.621 | −4.730 | −32.520 | 1.00 | 103.33 | | C |
| ANISOU | 1650 | C | LYS | C | 222 | 11819 | 15879 | 11561 | 3135 | 532 | −1372 | C |
| ATOM | 1651 | O | LYS | C | 222 | 77.982 | −4.858 | −33.567 | 1.00 | 103.45 | | O |
| ANISOU | 1651 | O | LYS | C | 222 | 11809 | 16063 | 11434 | 3224 | 618 | −1495 | O |
| ATOM | 1652 | CB | LYS | C | 222 | 80.710 | −6.146 | −32.997 | 1.00 | 106.84 | | C |
| ANISOU | 1652 | CB | LYS | C | 222 | 11919 | 16674 | 12002 | 3833 | 282 | −1851 | C |
| ATOM | 1653 | CG | LYS | C | 222 | 81.465 | −6.082 | −34.325 | 1.00 | 122.09 | | C |
| ANISOU | 1653 | CG | LYS | C | 222 | 13438 | 19237 | 13713 | 4137 | 430 | −2042 | C |
| ATOM | 1654 | CD | LYS | C | 222 | 81.918 | −7.470 | −34.825 | 1.00 | 134.07 | | C |
| ANISOU | 1654 | CD | LYS | C | 222 | 14939 | 20773 | 15228 | 4622 | 187 | −2486 | C |
| ATOM | 1655 | CE | LYS | C | 222 | 82.779 | −7.415 | −36.077 | 1.00 | 138.93 | | C |
| ANISOU | 1655 | CE | LYS | C | 222 | 15103 | 22072 | 15611 | 4943 | 327 | −2696 | C |
| ATOM | 1656 | NZ | LYS | C | 222 | 83.143 | −8.769 | −36.581 | 1.00 | 142.57 | | N |
| ANISOU | 1656 | NZ | LYS | C | 222 | 15555 | 22544 | 16070 | 5440 | 75 | −3161 | N |
| ATOM | 1657 | N | ALA | C | 223 | 78.032 | −4.504 | −31.323 | 1.00 | 96.98 | | N |
| ANISOU | 1657 | N | ALA | C | 223 | 11291 | 14644 | 10912 | 2825 | 459 | −1169 | N |
| ATOM | 1658 | CA | ALA | C | 223 | 76.596 | −4.357 | −31.096 | 1.00 | 93.48 | | C |
| ANISOU | 1658 | CA | ALA | C | 223 | 11127 | 13904 | 10486 | 2549 | 482 | −1072 | C |
| ATOM | 1659 | C | ALA | C | 223 | 76.269 | −2.875 | −30.869 | 1.00 | 92.66 | | C |
| ANISOU | 1659 | C | ALA | C | 223 | 10940 | 13940 | 10325 | 2225 | 725 | −748 | C |
| ATOM | 1660 | O | ALA | C | 223 | 75.238 | −2.400 | −31.341 | 1.00 | 90.13 | | O |
| ANISOU | 1660 | O | ALA | C | 223 | 10671 | 13669 | 9906 | 2098 | 854 | −695 | O |
| ATOM | 1661 | CB | ALA | C | 223 | 76.169 | −5.184 | −29.894 | 1.00 | 93.62 | | C |
| ANISOU | 1661 | CB | ALA | C | 223 | 11498 | 13380 | 10693 | 2421 | 234 | −1071 | C |
| ATOM | 1662 | N | ILE | C | 224 | 77.164 | −2.147 | −30.164 | 1.00 | 88.44 | | N |
| ANISOU | 1662 | N | ILE | C | 224 | 10275 | 13473 | 9853 | 2105 | 775 | −547 | N |
| ATOM | 1663 | CA | ILE | C | 224 | 77.059 | −0.715 | −29.869 | 1.00 | 86.49 | | C |
| ANISOU | 1663 | CA | ILE | C | 224 | 9947 | 13338 | 9579 | 1807 | 978 | −242 | C |
| ATOM | 1664 | C | ILE | C | 224 | 77.125 | 0.063 | −31.175 | 1.00 | 91.25 | | C |
| ANISOU | 1664 | C | ILE | C | 224 | 10300 | 14394 | 9975 | 1867 | 1217 | −198 | C |
| ATOM | 1665 | O | ILE | C | 224 | 76.238 | 0.877 | −31.439 | 1.00 | 88.96 | | O |
| ANISOU | 1665 | O | ILE | C | 224 | 10078 | 14115 | 9606 | 1688 | 1353 | −54 | O |
| ATOM | 1666 | CB | ILE | C | 224 | 78.153 | −0.276 | −28.847 | 1.00 | 90.20 | | C |
| ANISOU | 1666 | CB | ILE | C | 224 | 10310 | 13796 | 10168 | 1705 | 946 | −89 | C |
| ATOM | 1667 | CG1 | ILE | C | 224 | 77.736 | −0.642 | −27.408 | 1.00 | 89.55 | | C |
| ANISOU | 1667 | CG1 | ILE | C | 224 | 10524 | 13240 | 10263 | 1534 | 750 | −35 | C |
| ATOM | 1668 | CG2 | ILE | C | 224 | 78.502 | 1.227 | −28.957 | 1.00 | 90.43 | | C |
| ANISOU | 1668 | CG2 | ILE | C | 224 | 10142 | 14081 | 10136 | 1474 | 1180 | 185 | C |
| ATOM | 1669 | CD1 | ILE | C | 224 | 78.886 | −0.671 | −26.378 | 1.00 | 100.30 | | C |
| ANISOU | 1669 | CD1 | ILE | C | 224 | 11802 | 14563 | 11746 | 1541 | 636 | 19 | C |
| ATOM | 1670 | N | HIS | C | 225 | 78.162 | −0.224 | −32.005 | 1.00 | 90.96 | | N |
| ANISOU | 1670 | N | HIS | C | 225 | 9978 | 14745 | 9838 | 2132 | 1258 | −334 | N |
| ATOM | 1671 | CA | HIS | C | 225 | 78.378 | 0.394 | −33.314 | 1.00 | 91.78 | | C |
| ANISOU | 1671 | CA | HIS | C | 225 | 9820 | 15337 | 9714 | 2211 | 1482 | −302 | C |
| ATOM | 1672 | C | HIS | C | 225 | 77.290 | −0.040 | −34.297 | 1.00 | 94.95 | | C |
| ANISOU | 1672 | C | HIS | C | 225 | 10331 | 15766 | 9980 | 2338 | 1498 | −462 | C |
| ATOM | 1673 | O | HIS | C | 225 | 77.057 | 0.660 | −35.285 | 1.00 | 95.65 | | O |
| ANISOU | 1673 | O | HIS | C | 225 | 10299 | 16169 | 9874 | 2315 | 1692 | −363 | O |
| ATOM | 1674 | CB | HIS | C | 225 | 79.783 | 0.085 | −33.856 | 1.00 | 95.68 | | C |
| ANISOU | 1674 | CB | HIS | C | 225 | 9969 | 16260 | 10125 | 2472 | 1505 | −446 | C |
| ATOM | 1675 | N | LEU | C | 226 | 76.604 | −1.173 | −34.015 | 1.00 | 89.73 | | N |
| ANISOU | 1675 | N | LEU | C | 226 | 9910 | 14765 | 9419 | 2452 | 1289 | −696 | N |
| ATOM | 1676 | CA | LEU | C | 226 | 75.507 | −1.646 | −34.852 | 1.00 | 89.02 | | C |
| ANISOU | 1676 | CA | LEU | C | 226 | 9936 | 14666 | 9220 | 2549 | 1280 | −871 | C |
| ATOM | 1677 | C | LEU | C | 226 | 74.248 | −0.832 | −34.615 | 1.00 | 89.02 | | C |
| ANISOU | 1677 | C | LEU | C | 226 | 10124 | 14491 | 9211 | 2253 | 1369 | −669 | C |
| ATOM | 1678 | O | LEU | C | 226 | 73.604 | −0.442 | −35.584 | 1.00 | 88.84 | | O |
| ANISOU | 1678 | O | LEU | C | 226 | 10050 | 14698 | 9007 | 2282 | 1500 | −666 | O |
| ATOM | 1679 | CB | LEU | C | 226 | 75.227 | −3.144 | −34.675 | 1.00 | 90.20 | | C |
| ANISOU | 1679 | CB | LEU | C | 226 | 10281 | 14509 | 9482 | 2749 | 1020 | −1195 | C |
| ATOM | 1680 | CG | LEU | C | 226 | 75.340 | −4.000 | −35.951 | 1.00 | 97.78 | | C |
| ANISOU | 1680 | CG | LEU | C | 226 | 11102 | 15755 | 10293 | 3123 | 986 | −1532 | C |
| ATOM | 1681 | CD1 | LEU | C | 226 | 75.117 | −5.475 | −35.647 | 1.00 | 99.40 | | C |
| ANISOU | 1681 | CD1 | LEU | C | 226 | 11537 | 15584 | 10648 | 3304 | 694 | −1845 | C |
| ATOM | 1682 | CD2 | LEU | C | 226 | 74.388 | −3.527 | −37.049 | 1.00 | 99.13 | | C |
| ANISOU | 1682 | CD2 | LEU | C | 226 | 11229 | 16187 | 10251 | 3116 | 1153 | −1538 | C |
| ATOM | 1683 | N | LEU | C | 227 | 73.900 | −0.560 | −33.342 | 1.00 | 82.59 | | N |
| ANISOU | 1683 | N | LEU | C | 227 | 9514 | 13295 | 8573 | 1982 | 1295 | −509 | N |
| ATOM | 1684 | CA | LEU | C | 227 | 72.726 | 0.240 | −33.021 | 1.00 | 79.33 | | C |
| ANISOU | 1684 | CA | LEU | C | 227 | 9262 | 12727 | 8152 | 1711 | 1369 | −336 | C |
| ATOM | 1685 | C | LEU | C | 227 | 72.924 | 1.693 | −33.378 | 1.00 | 81.92 | | C |
| ANISOU | 1685 | C | LEU | C | 227 | 9437 | 13322 | 8366 | 1584 | 1587 | −61 | C |
| ATOM | 1686 | O | LEU | C | 227 | 72.042 | 2.271 | −34.005 | 1.00 | 80.30 | | O |
| ANISOU | 1686 | O | LEU | C | 227 | 9258 | 13227 | 8025 | 1548 | 1691 | −10 | O |
| ATOM | 1687 | CB | LEU | C | 227 | 72.287 | 0.089 | −31.568 | 1.00 | 77.34 | | C |
| ANISOU | 1687 | CB | LEU | C | 227 | 9257 | 12033 | 8097 | 1463 | 1231 | −260 | C |
| ATOM | 1688 | CG | LEU | C | 227 | 70.990 | −0.688 | −31.363 | 1.00 | 81.77 | | C |
| ANISOU | 1688 | CG | LEU | C | 227 | 10068 | 12313 | 8687 | 1389 | 1111 | −419 | C |
| ATOM | 1689 | CD1 | LEU | C | 227 | 70.574 | −0.672 | −29.926 | 1.00 | 80.30 | | C |
| ANISOU | 1689 | CD1 | LEU | C | 227 | 10095 | 11763 | 8652 | 1103 | 1018 | −296 | C |
| ATOM | 1690 | CD2 | LEU | C | 227 | 69.854 | −0.131 | −32.190 | 1.00 | 85.45 | | C |
| ANISOU | 1690 | CD2 | LEU | C | 227 | 10524 | 12953 | 8990 | 1364 | 1239 | −429 | C |
| ATOM | 1691 | N | GLN | C | 228 | 74.099 | 2.264 | −33.036 | 1.00 | 79.64 | | N |
| ANISOU | 1691 | N | GLN | C | 228 | 8985 | 13151 | 8123 | 1527 | 1648 | 109 | N |
| ATOM | 1692 | CA | GLN | C | 228 | 74.478 | 3.644 | −33.354 | 1.00 | 80.23 | | C |
| ANISOU | 1692 | CA | GLN | C | 228 | 8910 | 13471 | 8101 | 1383 | 1847 | 389 | C |
| ATOM | 1693 | C | GLN | C | 228 | 74.220 | 3.936 | −34.827 | 1.00 | 87.57 | | C |
| ANISOU | 1693 | C | GLN | C | 228 | 9714 | 14779 | 8779 | 1533 | 1998 | 370 | C |
| ATOM | 1694 | O | GLN | C | 228 | 73.600 | 4.951 | −35.142 | 1.00 | 86.93 | | O |
| ANISOU | 1694 | O | GLN | C | 228 | 9695 | 14733 | 8603 | 1403 | 2108 | 552 | O |
| ATOM | 1695 | CB | GLN | C | 228 | 75.960 | 3.904 | −33.027 | 1.00 | 83.15 | | C |
| ANISOU | 1695 | CB | GLN | C | 228 | 9055 | 14012 | 8526 | 1368 | 1884 | 488 | C |
| ATOM | 1696 | CG | GLN | C | 228 | 76.232 | 4.237 | −31.564 | 1.00 | 100.67 | | C |
| ANISOU | 1696 | CG | GLN | C | 228 | 11376 | 15909 | 10963 | 1137 | 1791 | 624 | C |
| ATOM | 1697 | N | GLU | C | 229 | 74.653 | 3.016 | −35.721 | 1.00 | 87.11 | | N |
| ANISOU | 1697 | N | GLU | C | 229 | 9495 | 14996 | 8608 | 1825 | 1985 | 131 | N |
| ATOM | 1698 | CA | GLU | C | 229 | 74.477 | 3.117 | −37.171 | 1.00 | 88.74 | | C |
| ANISOU | 1698 | CA | GLU | C | 229 | 9560 | 15608 | 8549 | 2008 | 2116 | 69 | C |
| ATOM | 1699 | C | GLU | C | 229 | 73.000 | 2.961 | −37.576 | 1.00 | 92.10 | | C |
| ANISOU | 1699 | C | GLU | C | 229 | 10186 | 15907 | 8902 | 2033 | 2081 | −33 | C |
| ATOM | 1700 | O | GLU | C | 229 | 72.528 | 3.688 | −38.453 | 1.00 | 93.19 | | O |
| ANISOU | 1700 | O | GLU | C | 229 | 10287 | 16284 | 8836 | 2038 | 2217 | 79 | O |
| ATOM | 1701 | CB | GLU | C | 229 | 75.366 | 2.094 | −37.904 | 1.00 | 92.75 | | C |
| ANISOU | 1701 | CB | GLU | C | 229 | 9844 | 16429 | 8966 | 2335 | 2081 | −213 | C |
| ATOM | 1702 | N | THR | C | 230 | 72.278 | 2.040 | −36.917 | 1.00 | 86.35 | | N |
| ANISOU | 1702 | N | THR | C | 230 | 9669 | 14809 | 8333 | 2033 | 1897 | −233 | N |
| ATOM | 1703 | CA | THR | C | 230 | 70.875 | 1.731 | −37.182 | 1.00 | 84.97 | | C |
| ANISOU | 1703 | CA | THR | C | 230 | 9671 | 14505 | 8110 | 2040 | 1841 | −375 | C |
| ATOM | 1704 | C | THR | C | 230 | 69.951 | 2.900 | −36.839 | 1.00 | 85.65 | | C |
| ANISOU | 1704 | C | THR | C | 230 | 9885 | 14475 | 8182 | 1794 | 1920 | −128 | C |
| ATOM | 1705 | O | THR | C | 230 | 69.182 | 3.344 | −37.693 | 1.00 | 84.39 | | O |
| ANISOU | 1705 | O | THR | C | 230 | 9717 | 14510 | 7836 | 1849 | 2006 | −109 | O |
| ATOM | 1706 | CB | THR | C | 230 | 70.513 | 0.426 | −36.466 | 1.00 | 98.02 | | C |
| ANISOU | 1706 | CB | THR | C | 230 | 11506 | 15791 | 9945 | 2066 | 1620 | −637 | C |
| ATOM | 1707 | CG2 | THR | C | 230 | 69.027 | 0.071 | −36.566 | 1.00 | 97.04 | | C |
| ANISOU | 1707 | CG2 | THR | C | 230 | 11565 | 15514 | 9792 | 2021 | 1552 | −793 | C |
| ATOM | 1708 | OG1 | THR | C | 230 | 71.310 | −0.617 | −37.031 | 1.00 | 102.87 | | O |
| ANISOU | 1708 | OG1 | THR | C | 230 | 11997 | 16551 | 10540 | 2354 | 1540 | −891 | O |
| ATOM | 1709 | N | LEU | C | 231 | 70.024 | 3.379 | −35.586 | 1.00 | 81.02 | | N |
| ANISOU | 1709 | N | LEU | C | 231 | 9417 | 13579 | 7787 | 1545 | 1878 | 45 | N |
| ATOM | 1710 | CA | LEU | C | 231 | 69.217 | 4.479 | −35.053 | 1.00 | 78.50 | | C |
| ANISOU | 1710 | CA | LEU | C | 231 | 9229 | 13105 | 7492 | 1312 | 1922 | 258 | C |
| ATOM | 1711 | C | LEU | C | 231 | 69.371 | 5.774 | −35.857 | 1.00 | 83.00 | | C |
| ANISOU | 1711 | C | LEU | C | 231 | 9700 | 13952 | 7885 | 1303 | 2097 | 507 | C |
| ATOM | 1712 | O | LEU | C | 231 | 68.356 | 6.345 | −36.267 | 1.00 | 82.49 | | O |
| ANISOU | 1712 | O | LEU | C | 231 | 9719 | 13917 | 7705 | 1297 | 2129 | 540 | O |
| ATOM | 1713 | CB | LEU | C | 231 | 69.545 | 4.716 | −33.570 | 1.00 | 76.67 | | C |
| ANISOU | 1713 | CB | LEU | C | 231 | 9095 | 12550 | 7488 | 1075 | 1850 | 394 | C |
| ATOM | 1714 | CG | LEU | C | 231 | 69.280 | 3.553 | −32.613 | 1.00 | 79.85 | | C |
| ANISOU | 1714 | CG | LEU | C | 231 | 9648 | 12630 | 8062 | 1030 | 1668 | 203 | C |
| ATOM | 1715 | CD1 | LEU | C | 231 | 69.987 | 3.758 | −31.312 | 1.00 | 78.69 | | C |
| ANISOU | 1715 | CD1 | LEU | C | 231 | 9532 | 12265 | 8103 | 861 | 1608 | 342 | C |
| ATOM | 1716 | CD2 | LEU | C | 231 | 67.816 | 3.374 | −32.357 | 1.00 | 80.99 | | C |
| ANISOU | 1716 | CD2 | LEU | C | 231 | 9977 | 12589 | 8207 | 914 | 1606 | 108 | C |
| ATOM | 1717 | N | ALA | C | 232 | 70.636 | 6.201 | −36.119 | 1.00 | 80.08 | | N |
| ANISOU | 1717 | N | ALA | C | 232 | 9149 | 13799 | 7479 | 1309 | 2202 | 672 | N |
| ATOM | 1718 | CA | ALA | C | 232 | 70.996 | 7.414 | −36.870 | 1.00 | 80.65 | | C |
| ANISOU | 1718 | CA | ALA | C | 232 | 9123 | 14139 | 7383 | 1263 | 2372 | 948 | C |
| ATOM | 1719 | C | ALA | C | 232 | 70.426 | 7.442 | −38.286 | 1.00 | 84.73 | | C |
| ANISOU | 1719 | C | ALA | C | 232 | 9590 | 14981 | 7622 | 1463 | 2455 | 891 | C |
| ATOM | 1720 | O | ALA | C | 232 | 70.071 | 8.514 | −38.777 | 1.00 | 84.27 | | O |
| ANISOU | 1720 | O | ALA | C | 232 | 9574 | 15020 | 7426 | 1403 | 2549 | 1117 | O |
| ATOM | 1721 | CB | ALA | C | 232 | 72.508 | 7.572 | −36.920 | 1.00 | 83.18 | | C |
| ANISOU | 1721 | CB | ALA | C | 232 | 9223 | 14670 | 7711 | 1232 | 2460 | 1073 | C |
| ATOM | 1722 | N | ARG | C | 233 | 70.339 | 6.261 | −38.929 | 1.00 | 81.88 | | N |
| ANISOU | 1722 | N | ARG | C | 233 | 9150 | 14780 | 7179 | 1709 | 2405 | 584 | N |
| ATOM | 1723 | CA | ARG | C | 233 | 69.816 | 6.064 | −40.282 | 1.00 | 82.85 | | C |
| ANISOU | 1723 | CA | ARG | C | 233 | 9209 | 15238 | 7034 | 1937 | 2462 | 461 | C |
| ATOM | 1724 | C | ARG | C | 233 | 68.283 | 6.276 | −40.388 | 1.00 | 85.66 | | C |
| ANISOU | 1724 | C | ARG | C | 233 | 9755 | 15456 | 7336 | 1932 | 2406 | 406 | C |
| ATOM | 1725 | O | ARG | C | 233 | 67.795 | 6.589 | −41.480 | 1.00 | 87.10 | | O |
| ANISOU | 1725 | O | ARG | C | 233 | 9906 | 15917 | 7271 | 2068 | 2478 | 424 | O |
| ATOM | 1726 | CB | ARG | C | 233 | 70.236 | 4.686 | −40.826 | 1.00 | 83.10 | | C |
| ANISOU | 1726 | CB | ARG | C | 233 | 9104 | 15446 | 7025 | 2203 | 2397 | 110 | C |
| ATOM | 1727 | N | ASP | C | 234 | 67.532 | 6.115 | −39.268 | 1.00 | 78.91 | | N |
| ANISOU | 1727 | N | ASP | C | 234 | 9084 | 14205 | 6694 | 1780 | 2278 | 337 | N |
| ATOM | 1728 | CA | ASP | C | 234 | 66.079 | 6.305 | −39.238 | 1.00 | 77.09 | | C |
| ANISOU | 1728 | CA | ASP | C | 234 | 9009 | 13855 | 6425 | 1760 | 2218 | 261 | C |
| ATOM | 1729 | C | ASP | C | 234 | 65.723 | 7.786 | −39.396 | 1.00 | 81.10 | | C |
| ANISOU | 1729 | C | ASP | C | 234 | 9586 | 14390 | 6838 | 1665 | 2301 | 569 | C |
| ATOM | 1730 | O | ASP | C | 234 | 66.192 | 8.622 | −38.620 | 1.00 | 79.92 | | O |
| ANISOU | 1730 | O | ASP | C | 234 | 9482 | 14066 | 6818 | 1469 | 2328 | 819 | O |
| ATOM | 1731 | CB | ASP | C | 234 | 65.472 | 5.723 | −37.947 | 1.00 | 76.59 | | C |
| ANISOU | 1731 | CB | ASP | C | 234 | 9100 | 13399 | 6602 | 1596 | 2071 | 112 | C |
| ATOM | 1732 | CG | ASP | C | 234 | 63.962 | 5.874 | −37.820 | 1.00 | 82.14 | | C |
| ANISOU | 1732 | CG | ASP | C | 234 | 9932 | 14009 | 7267 | 1558 | 2007 | −1 | C |
| ATOM | 1733 | OD1 | ASP | C | 234 | 63.228 | 5.390 | −38.723 | 1.00 | 82.68 | | O |
| ANISOU | 1733 | OD1 | ASP | C | 234 | 9972 | 14260 | 7184 | 1729 | 1984 | −228 | O |
| ATOM | 1734 | OD2 | ASP | C | 234 | 63.514 | 6.445 | −36.814 | 1.00 | 83.62 | | O |
| ANISOU | 1734 | OD2 | ASP | C | 234 | 10236 | 13964 | 7571 | 1364 | 1975 | 114 | O |
| ATOM | 1735 | N | GLU | C | 235 | 64.893 | 8.099 | −40.412 | 1.00 | 78.89 | | N |
| ANISOU | 1735 | N | GLU | C | 235 | 9322 | 14322 | 6329 | 1816 | 2327 | 541 | N |
| ATOM | 1736 | CA | GLU | C | 235 | 64.458 | 9.462 | −40.732 | 1.00 | 78.75 | | C |
| ANISOU | 1736 | CA | GLU | C | 235 | 9395 | 14341 | 6187 | 1782 | 2381 | 814 | C |
| ATOM | 1737 | C | GLU | C | 235 | 63.340 | 10.005 | −39.793 | 1.00 | 80.60 | | C |
| ANISOU | 1737 | C | GLU | C | 235 | 9812 | 14262 | 6551 | 1654 | 2275 | 806 | C |
| ATOM | 1738 | O | GLU | C | 235 | 63.191 | 11.229 | −39.735 | 1.00 | 80.86 | | O |
| ANISOU | 1738 | O | GLU | C | 235 | 9944 | 14223 | 6556 | 1586 | 2295 | 1061 | O |
| ATOM | 1739 | CB | GLU | C | 235 | 64.072 | 9.599 | −42.219 | 1.00 | 81.96 | | C |
| ANISOU | 1739 | CB | GLU | C | 235 | 9740 | 15132 | 6268 | 2022 | 2444 | 800 | C |
| ATOM | 1740 | N | LEU | C | 236 | 62.581 | 9.138 | −39.050 | 1.00 | 74.56 | | N |
| ANISOU | 1740 | N | LEU | C | 236 | 9092 | 13318 | 5920 | 1614 | 2160 | 519 | N |
| ATOM | 1741 | CA | LEU | C | 236 | 61.556 | 9.647 | −38.107 | 1.00 | 72.37 | | C |
| ANISOU | 1741 | CA | LEU | C | 236 | 8955 | 12792 | 5751 | 1483 | 2069 | 495 | C |
| ATOM | 1742 | C | LEU | C | 236 | 61.974 | 9.572 | −36.640 | 1.00 | 73.13 | | C |
| ANISOU | 1742 | C | LEU | C | 236 | 9112 | 12558 | 6117 | 1234 | 2021 | 544 | C |
| ATOM | 1743 | O | LEU | C | 236 | 61.126 | 9.650 | −35.749 | 1.00 | 69.91 | | O |
| ANISOU | 1743 | O | LEU | C | 236 | 8795 | 11961 | 5808 | 1114 | 1937 | 451 | O |
| ATOM | 1744 | CB | LEU | C | 236 | 60.149 | 9.042 | −38.290 | 1.00 | 71.97 | | C |
| ANISOU | 1744 | CB | LEU | C | 236 | 8920 | 12802 | 5622 | 1574 | 1980 | 175 | C |
| ATOM | 1745 | CG | LEU | C | 236 | 59.896 | 7.645 | −37.792 | 1.00 | 74.42 | | C |
| ANISOU | 1745 | CG | LEU | C | 236 | 9208 | 13013 | 6053 | 1509 | 1902 | −125 | C |
| ATOM | 1746 | CD1 | LEU | C | 236 | 58.559 | 7.570 | −37.053 | 1.00 | 70.80 | | C |
| ANISOU | 1746 | CD1 | LEU | C | 236 | 8825 | 12425 | 5651 | 1390 | 1804 | −315 | C |
| ATOM | 1747 | CD2 | LEU | C | 236 | 60.125 | 6.616 | −38.939 | 1.00 | 78.69 | | C |
| ANISOU | 1747 | CD2 | LEU | C | 236 | 9633 | 13811 | 6453 | 1723 | 1919 | −327 | C |
| ATOM | 1748 | N | LEU | C | 237 | 63.269 | 9.428 | −36.395 | 1.00 | 71.14 | | N |
| ANISOU | 1748 | N | LEU | C | 237 | 8794 | 12268 | 5967 | 1162 | 2072 | 677 | N |
| ATOM | 1749 | CA | LEU | C | 237 | 63.808 | 9.429 | −35.041 | 1.00 | 70.31 | | C |
| ANISOU | 1749 | CA | LEU | C | 237 | 8736 | 11875 | 6104 | 939 | 2028 | 750 | C |
| ATOM | 1750 | C | LEU | C | 237 | 64.520 | 10.757 | −34.936 | 1.00 | 74.85 | | C |
| ANISOU | 1750 | C | LEU | C | 237 | 9325 | 12416 | 6698 | 843 | 2101 | 1083 | C |
| ATOM | 1751 | O | LEU | C | 237 | 64.234 | 11.555 | −34.048 | 1.00 | 72.58 | | O |
| ANISOU | 1751 | O | LEU | C | 237 | 9145 | 11905 | 6527 | 687 | 2055 | 1190 | O |
| ATOM | 1752 | CB | LEU | C | 237 | 64.813 | 8.278 | −34.857 | 1.00 | 70.76 | | C |
| ANISOU | 1752 | CB | LEU | C | 237 | 8699 | 11927 | 6259 | 951 | 2015 | 645 | C |
| ATOM | 1753 | CG | LEU | C | 237 | 64.930 | 7.649 | −33.475 | 1.00 | 73.48 | | C |
| ANISOU | 1753 | CG | LEU | C | 237 | 9117 | 11969 | 6831 | 770 | 1910 | 566 | C |
| ATOM | 1754 | CD1 | LEU | C | 237 | 63.616 | 7.011 | −33.043 | 1.00 | 72.37 | | C |
| ANISOU | 1754 | CD1 | LEU | C | 237 | 9090 | 11696 | 6712 | 711 | 1807 | 336 | C |
| ATOM | 1755 | CD2 | LEU | C | 237 | 66.024 | 6.594 | −33.472 | 1.00 | 76.00 | | C |
| ANISOU | 1755 | CD2 | LEU | C | 237 | 9347 | 12309 | 7222 | 843 | 1885 | 469 | C |
| ATOM | 1756 | N | GLN | C | 238 | 65.373 | 11.030 | −35.933 | 1.00 | 73.89 | | N |
| ANISOU | 1756 | N | GLN | C | 238 | 9095 | 12541 | 6438 | 937 | 2213 | 1240 | N |
| ATOM | 1757 | CA | GLN | C | 238 | 66.152 | 12.249 | −36.040 | 1.00 | 75.02 | | C |
| ANISOU | 1757 | CA | GLN | C | 238 | 9242 | 12691 | 6572 | 829 | 2297 | 1576 | C |
| ATOM | 1758 | C | GLN | C | 238 | 65.242 | 13.450 | −36.241 | 1.00 | 78.51 | | C |
| ANISOU | 1758 | C | GLN | C | 238 | 9841 | 13061 | 6928 | 836 | 2273 | 1719 | C |
| ATOM | 1759 | O | GLN | C | 238 | 65.689 | 14.581 | −36.047 | 1.00 | 79.30 | | O |
| ANISOU | 1759 | O | GLN | C | 238 | 10008 | 13055 | 7068 | 703 | 2301 | 1998 | O |
| ATOM | 1760 | CB | GLN | C | 238 | 67.194 | 12.135 | −37.179 | 1.00 | 79.10 | | C |
| ANISOU | 1760 | CB | GLN | C | 238 | 9584 | 13552 | 6918 | 927 | 2433 | 1688 | C |
| ATOM | 1761 | CG | GLN | C | 238 | 68.182 | 10.943 | −37.098 | 1.00 | 91.87 | | C |
| ANISOU | 1761 | CG | GLN | C | 238 | 11021 | 15282 | 8604 | 969 | 2449 | 1530 | C |
| ATOM | 1762 | CD | GLN | C | 238 | 68.573 | 10.525 | −35.697 | 1.00 | 103.65 | | C |
| ANISOU | 1762 | CD | GLN | C | 238 | 12545 | 16469 | 10368 | 810 | 2352 | 1454 | C |
| ATOM | 1763 | NE2 | GLN | C | 238 | 69.397 | 11.321 | −35.025 | 1.00 | 97.27 | | N |
| ANISOU | 1763 | NE2 | GLN | C | 238 | 11734 | 15528 | 9697 | 603 | 2375 | 1681 | N |
| ATOM | 1764 | OE1 | GLN | C | 238 | 68.104 | 9.506 | −35.188 | 1.00 | 95.26 | | O |
| ANISOU | 1764 | OE1 | GLN | C | 238 | 11523 | 15280 | 9392 | 860 | 2246 | 1196 | O |
| ATOM | 1765 | N | SER | C | 239 | 63.962 | 13.204 | −36.596 | 1.00 | 73.93 | | N |
| ANISOU | 1765 | N | SER | C | 239 | 9325 | 12529 | 6238 | 991 | 2207 | 1516 | N |
| ATOM | 1766 | CA | SER | C | 239 | 62.970 | 14.253 | −36.799 | 1.00 | 74.35 | | C |
| ANISOU | 1766 | CA | SER | C | 239 | 9525 | 12527 | 6198 | 1050 | 2155 | 1599 | C |
| ATOM | 1767 | C | SER | C | 239 | 62.353 | 14.702 | −35.480 | 1.00 | 79.52 | | C |
| ANISOU | 1767 | C | SER | C | 239 | 10305 | 12863 | 7048 | 905 | 2041 | 1549 | C |
| ATOM | 1768 | O | SER | C | 239 | 62.484 | 15.863 | −35.091 | 1.00 | 79.39 | | O |
| ANISOU | 1768 | O | SER | C | 239 | 10397 | 12656 | 7110 | 795 | 2018 | 1769 | O |
| ATOM | 1769 | CB | SER | C | 239 | 61.886 | 13.802 | −37.776 | 1.00 | 76.75 | | C |
| ANISOU | 1769 | CB | SER | C | 239 | 9812 | 13071 | 6279 | 1297 | 2130 | 1387 | C |
| ATOM | 1770 | OG | SER | C | 239 | 60.927 | 12.909 | −37.234 | 1.00 | 76.08 | | O |
| ANISOU | 1770 | OG | SER | C | 239 | 9710 | 12941 | 6255 | 1316 | 2042 | 1048 | O |
| ATOM | 1771 | N | ALA | C | 240 | 61.691 | 13.775 | −34.793 | 1.00 | 76.40 | | N |
| ANISOU | 1771 | N | ALA | C | 240 | 9889 | 12414 | 6724 | 892 | 1967 | 1255 | N |
| ATOM | 1772 | CA | ALA | C | 240 | 61.007 | 14.042 | −33.551 | 1.00 | 75.03 | | C |
| ANISOU | 1772 | CA | ALA | C | 240 | 9804 | 12004 | 6701 | 760 | 1864 | 1157 | C |
| ATOM | 1773 | C | ALA | C | 240 | 61.510 | 13.054 | −32.486 | 1.00 | 80.45 | | C |
| ANISOU | 1773 | C | ALA | C | 240 | 10438 | 12554 | 7574 | 581 | 1847 | 1040 | C |
| ATOM | 1774 | O | ALA | C | 240 | 60.738 | 12.145 | −32.168 | 1.00 | 80.29 | | O |
| ANISOU | 1774 | O | ALA | C | 240 | 10402 | 12550 | 7556 | 575 | 1793 | 780 | O |
| ATOM | 1775 | CB | ALA | C | 240 | 59.496 | 13.912 | −33.762 | 1.00 | 75.09 | | C |
| ANISOU | 1775 | CB | ALA | C | 240 | 9834 | 12113 | 6582 | 900 | 1788 | 905 | C |
| ATOM | 1776 | N | ALA | C | 241 | 62.782 | 13.185 | −31.907 | 1.00 | 77.23 | | N |
| ANISOU | 1776 | N | ALA | C | 241 | 10007 | 12017 | 7322 | 425 | 1883 | 1225 | N |
| ATOM | 1777 | CA | ALA | C | 241 | 63.887 | 14.155 | −32.141 | 1.00 | 77.85 | | C |
| ANISOU | 1777 | CA | ALA | C | 241 | 10081 | 12068 | 7429 | 362 | 1954 | 1536 | C |
| ATOM | 1778 | C | ALA | C | 241 | 65.292 | 13.711 | −31.575 | 1.00 | 81.03 | | C |
| ANISOU | 1778 | C | ALA | C | 241 | 10388 | 12409 | 7992 | 211 | 1990 | 1621 | C |
| ATOM | 1779 | O | ALA | C | 241 | 65.439 | 12.571 | −31.117 | 1.00 | 78.18 | | O |
| ANISOU | 1779 | O | ALA | C | 241 | 9975 | 12024 | 7705 | 193 | 1954 | 1440 | O |
| ATOM | 1780 | CB | ALA | C | 241 | 63.511 | 15.529 | −31.619 | 1.00 | 78.57 | | C |
| ANISOU | 1780 | CB | ALA | C | 241 | 10315 | 11961 | 7577 | 284 | 1895 | 1684 | C |
| ATOM | 1781 | N | ALA | C | 242 | 66.296 | 14.654 | −31.630 | 1.00 | 80.25 | | N |
| ANISOU | 1781 | N | ALA | C | 242 | 10272 | 12285 | 7935 | 105 | 2052 | 1899 | N |
| ATOM | 1782 | CA | ALA | C | 242 | 67.754 | 14.699 | −31.298 | 1.00 | 81.81 | | C |
| ANISOU | 1782 | CA | ALA | C | 242 | 10360 | 12471 | 8254 | −44 | 2106 | 2056 | C |
| ATOM | 1783 | C | ALA | C | 242 | 68.417 | 13.572 | −30.389 | 1.00 | 89.53 | | C |
| ANISOU | 1783 | C | ALA | C | 242 | 11241 | 13393 | 9381 | −101 | 2061 | 1897 | C |
| ATOM | 1784 | O | ALA | C | 242 | 67.739 | 12.833 | −29.671 | 1.00 | 89.40 | | O |
| ANISOU | 1784 | O | ALA | C | 242 | 11277 | 13284 | 9409 | −74 | 1974 | 1674 | O |
| ATOM | 1785 | CB | ALA | C | 242 | 68.091 | 16.053 | −30.685 | 1.00 | 82.46 | | C |
| ANISOU | 1785 | CB | ALA | C | 242 | 10538 | 12331 | 8462 | −242 | 2080 | 2284 | C |
| ATOM | 1786 | N | ALA | C | 243 | 69.795 | 13.498 | −30.469 | 1.00 | 87.57 | | N |
| ANISOU | 1786 | N | ALA | C | 243 | 10853 | 13221 | 9200 | −185 | 2121 | 2030 | N |
| ATOM | 1787 | CA | ALA | C | 243 | 70.848 | 12.672 | −29.813 | 1.00 | 87.04 | | C |
| ANISOU | 1787 | CA | ALA | C | 243 | 10660 | 13146 | 9264 | −233 | 2092 | 1963 | C |
| ATOM | 1788 | C | ALA | C | 243 | 70.473 | 11.286 | −29.174 | 1.00 | 88.28 | | C |
| ANISOU | 1788 | C | ALA | C | 243 | 10844 | 13212 | 9484 | −149 | 1981 | 1689 | C |
| ATOM | 1789 | O | ALA | C | 243 | 69.594 | 11.185 | −28.316 | 1.00 | 85.39 | | O |
| ANISOU | 1789 | O | ALA | C | 243 | 10625 | 12637 | 9182 | −211 | 1884 | 1582 | O |
| ATOM | 1790 | CB | ALA | C | 243 | 71.568 | 13.501 | −28.763 | 1.00 | 87.72 | | C |
| ANISOU | 1790 | CB | ALA | C | 243 | 10763 | 13034 | 9532 | −468 | 2055 | 2119 | C |
| ATOM | 1791 | N | ALA | C | 244 | 71.281 | 10.256 | −29.528 | 1.00 | 86.28 | | N |
| ANISOU | 1791 | N | ALA | C | 244 | 10444 | 13116 | 9223 | −28 | 1989 | 1591 | N |
| ATOM | 1792 | CA | ALA | C | 244 | 71.216 | 8.857 | −29.077 | 1.00 | 86.13 | | C |
| ANISOU | 1792 | CA | ALA | C | 244 | 10443 | 13018 | 9265 | 71 | 1875 | 1352 | C |
| ATOM | 1793 | C | ALA | C | 244 | 72.414 | 8.540 | −28.111 | 1.00 | 92.62 | | C |
| ANISOU | 1793 | C | ALA | C | 244 | 11193 | 13743 | 10254 | −9 | 1801 | 1376 | C |
| ATOM | 1794 | O | ALA | C | 244 | 73.096 | 9.488 | −27.718 | 1.00 | 92.61 | | O |
| ANISOU | 1794 | O | ALA | C | 244 | 11135 | 13729 | 10324 | −168 | 1843 | 1566 | O |
| ATOM | 1795 | CB | ALA | C | 244 | 71.238 | 7.938 | −30.295 | 1.00 | 88.24 | | C |
| ANISOU | 1795 | CB | ALA | C | 244 | 10601 | 13545 | 9381 | 324 | 1917 | 1194 | C |
| ATOM | 1796 | N | ALA | C | 245 | 72.646 | 7.234 | −27.696 | 1.00 | 90.88 | | N |
| ANISOU | 1796 | N | ALA | C | 245 | 10990 | 13439 | 10101 | 97 | 1676 | 1183 | N |
| ATOM | 1797 | CA | ALA | C | 245 | 73.747 | 6.800 | −26.787 | 1.00 | 91.28 | | C |
| ANISOU | 1797 | CA | ALA | C | 245 | 10985 | 13401 | 10298 | 65 | 1576 | 1180 | C |
| ATOM | 1798 | C | ALA | C | 245 | 74.059 | 5.247 | −26.737 | 1.00 | 98.28 | | C |
| ANISOU | 1798 | C | ALA | C | 245 | 11883 | 14241 | 11218 | 269 | 1433 | 950 | C |
| ATOM | 1799 | O | ALA | C | 245 | 73.480 | 4.454 | −27.480 | 1.00 | 98.14 | | O |
| ANISOU | 1799 | O | ALA | C | 245 | 11898 | 14281 | 11111 | 443 | 1419 | 778 | O |
| ATOM | 1800 | CB | ALA | C | 245 | 73.484 | 7.314 | −25.375 | 1.00 | 89.93 | | C |
| ANISOU | 1800 | CB | ALA | C | 245 | 10963 | 12944 | 10263 | −175 | 1493 | 1272 | C |
| ATOM | 1801 | N | GLY | C | 246 | 75.036 | 4.886 | −25.894 | 1.00 | 97.37 | | N |
| ANISOU | 1801 | N | GLY | C | 246 | 11737 | 14028 | 11230 | 254 | 1321 | 951 | N |
| ATOM | 1802 | CA | GLY | C | 246 | 75.473 | 3.540 | −25.497 | 1.00 | 99.12 | | C |
| ANISOU | 1802 | CA | GLY | C | 246 | 12009 | 14127 | 11526 | 417 | 1139 | 774 | C |
| ATOM | 1803 | C | GLY | C | 246 | 75.449 | 3.518 | −23.962 | 1.00 | 104.64 | | C |
| ANISOU | 1803 | C | GLY | C | 246 | 12852 | 14537 | 12369 | 231 | 1000 | 852 | C |
| ATOM | 1804 | O | GLY | C | 246 | 74.675 | 4.296 | −23.385 | 1.00 | 103.16 | | O |
| ANISOU | 1804 | O | GLY | C | 246 | 12764 | 14228 | 12202 | −7 | 1042 | 985 | O |
| ATOM | 1805 | N | PRO | C | 247 | 76.213 | 2.704 | −23.183 | 1.00 | 102.74 | | N |
| ANISOU | 1805 | N | PRO | C | 247 | 12643 | 14168 | 12224 | 323 | 820 | 777 | N |
| ATOM | 1806 | CA | PRO | C | 247 | 77.282 | 1.744 | −23.493 | 1.00 | 104.77 | | C |
| ANISOU | 1806 | CA | PRO | C | 247 | 12797 | 14516 | 12494 | 617 | 707 | 606 | C |
| ATOM | 1807 | C | PRO | C | 247 | 76.912 | 0.289 | −23.093 | 1.00 | 107.47 | | C |
| ANISOU | 1807 | C | PRO | C | 247 | 13409 | 14535 | 12891 | 719 | 475 | 446 | C |
| ATOM | 1808 | O | PRO | C | 247 | 76.332 | −0.410 | −23.912 | 1.00 | 106.05 | | O |
| ANISOU | 1808 | O | PRO | C | 247 | 13313 | 14331 | 12650 | 842 | 461 | 294 | O |
| ATOM | 1809 | CB | PRO | C | 247 | 78.436 | 2.298 | −22.630 | 1.00 | 107.74 | | C |
| ANISOU | 1809 | CB | PRO | C | 247 | 12998 | 14981 | 12957 | 579 | 677 | 706 | C |
| ATOM | 1810 | CG | PRO | C | 247 | 77.696 | 3.023 | −21.408 | 1.00 | 110.33 | | C |
| ANISOU | 1810 | CG | PRO | C | 247 | 13495 | 15066 | 13358 | 246 | 665 | 892 | C |
| ATOM | 1811 | CD | PRO | C | 247 | 76.203 | 2.926 | −21.728 | 1.00 | 103.82 | | C |
| ANISOU | 1811 | CD | PRO | C | 247 | 12893 | 14080 | 12472 | 122 | 713 | 889 | C |
| ATOM | 1812 | N | ALA | C | 248 | 77.231 | −0.152 | −21.825 | 1.00 | 104.68 | | N |
| ANISOU | 1812 | N | ALA | C | 248 | 13202 | 13926 | 12647 | 654 | 287 | 486 | N |
| ATOM | 1813 | CA | ALA | C | 248 | 76.984 | −1.484 | −21.242 | 1.00 | 105.43 | | C |
| ANISOU | 1813 | CA | ALA | C | 248 | 13586 | 13672 | 12801 | 713 | 39 | 384 | C |
| ATOM | 1814 | C | ALA | C | 248 | 76.718 | −1.494 | −19.717 | 1.00 | 108.46 | | C |
| ANISOU | 1814 | C | ALA | C | 248 | 14193 | 13764 | 13253 | 457 | −83 | 533 | C |
| ATOM | 1815 | O | ALA | C | 248 | 75.992 | −2.376 | −19.248 | 1.00 | 108.12 | | O |
| ANISOU | 1815 | O | ALA | C | 248 | 14444 | 13416 | 13221 | 375 | −228 | 505 | O |
| ATOM | 1816 | CB | ALA | C | 248 | 78.136 | −2.420 | −21.566 | 1.00 | 109.10 | | C |
| ANISOU | 1816 | CB | ALA | C | 248 | 13967 | 14180 | 13307 | 1065 | −131 | 210 | C |
| ATOM | 1817 | N | ALA | C | 249 | 77.342 | −0.548 | −18.954 | 1.00 | 104.70 | | N |
| ANISOU | 1817 | N | ALA | C | 249 | 13576 | 13391 | 12816 | 329 | −33 | 686 | N |
| ATOM | 1818 | CA | ALA | C | 249 | 77.256 | −0.311 | −17.488 | 1.00 | 103.89 | | C |
| ANISOU | 1818 | CA | ALA | C | 249 | 13619 | 13094 | 12762 | 93 | −126 | 833 | C |
| ATOM | 1819 | C | ALA | C | 249 | 78.029 | −1.330 | −16.589 | 1.00 | 109.82 | | C |
| ANISOU | 1819 | C | ALA | C | 249 | 14509 | 13635 | 13581 | 223 | −400 | 804 | C |
| ATOM | 1820 | O | ALA | C | 249 | 78.996 | −0.917 | −15.943 | 1.00 | 109.58 | | O |
| ANISOU | 1820 | O | ALA | C | 249 | 14354 | 13683 | 13600 | 221 | −448 | 875 | O |
| ATOM | 1821 | CB | ALA | C | 249 | 75.806 | −0.181 | −17.021 | 1.00 | 102.74 | | C |
| ANISOU | 1821 | CB | ALA | C | 249 | 13710 | 12762 | 12567 | −197 | −84 | 903 | C |
| ATOM | 1822 | N | ALA | C | 250 | 77.583 | −2.624 | −16.519 | 1.00 | 107.94 | | N |
| ANISOU | 1822 | N | ALA | C | 250 | 14541 | 13123 | 13346 | 327 | −588 | 704 | N |
| ATOM | 1823 | CA | ALA | C | 250 | 78.154 | −3.767 | −15.749 | 1.00 | 110.05 | | C |
| ANISOU | 1823 | CA | ALA | C | 250 | 15016 | 13127 | 13670 | 479 | −886 | 670 | C |
| ATOM | 1824 | C | ALA | C | 250 | 78.148 | −3.604 | −14.189 | 1.00 | 112.21 | | C |
| ANISOU | 1824 | C | ALA | C | 250 | 15471 | 13211 | 13955 | 246 | −1010 | 849 | C |
| ATOM | 1825 | O | ALA | C | 250 | 79.204 | −3.419 | −13.561 | 1.00 | 113.00 | | O |
| ANISOU | 1825 | O | ALA | C | 250 | 15460 | 13376 | 14098 | 345 | −1113 | 885 | O |
| ATOM | 1826 | CB | ALA | C | 250 | 79.558 | −4.122 | −16.247 | 1.00 | 113.27 | | C |
| ANISOU | 1826 | CB | ALA | C | 250 | 15188 | 13721 | 14127 | 861 | −979 | 528 | C |
| ATOM | 1827 | N | ALA | C | 251 | 76.948 | −3.748 | −13.575 | 1.00 | 105.80 | | N |
| ANISOU | 1827 | N | ALA | C | 251 | 14934 | 12178 | 13088 | −55 | −1011 | 947 | N |
| ATOM | 1828 | CA | ALA | C | 251 | 76.732 | −3.624 | −12.124 | 1.00 | 104.04 | | C |
| ANISOU | 1828 | CA | ALA | C | 251 | 14904 | 11790 | 12838 | −305 | −1115 | 1115 | C |
| ATOM | 1829 | C | ALA | C | 251 | 76.943 | −4.950 | −11.357 | 1.00 | 107.36 | | C |
| ANISOU | 1829 | C | ALA | C | 251 | 15656 | 11864 | 13273 | −213 | −1429 | 1130 | C |
| ATOM | 1830 | O | ALA | C | 251 | 76.121 | −5.862 | −11.448 | 1.00 | 107.73 | | O |
| ANISOU | 1830 | O | ALA | C | 251 | 15961 | 11665 | 13306 | −224 | −1524 | 1078 | O |
| ATOM | 1831 | CB | ALA | C | 251 | 75.351 | −3.024 | −11.838 | 1.00 | 102.41 | | C |
| ANISOU | 1831 | CB | ALA | C | 251 | 14803 | 11567 | 12541 | −676 | −955 | 1203 | C |
| ATOM | 1832 | N | LEU | C | 252 | 78.053 | −5.041 | −10.596 | 1.00 | 103.38 | | N |
| ANISOU | 1832 | N | LEU | C | 252 | 15148 | 11334 | 12796 | −119 | −1601 | 1198 | N |
| ATOM | 1833 | CA | LEU | C | 252 | 78.446 | −6.221 | −9.798 | 1.00 | 105.12 | | C |
| ANISOU | 1833 | CA | LEU | C | 252 | 15686 | 11227 | 13026 | 0 | −1929 | 1233 | C |
| ATOM | 1834 | C | LEU | C | 252 | 77.549 | −6.445 | −8.553 | 1.00 | 107.81 | | C |
| ANISOU | 1834 | C | LEU | C | 252 | 16373 | 11322 | 13266 | −367 | −2007 | 1429 | C |
| ATOM | 1835 | O | LEU | C | 252 | 76.480 | −5.830 | −8.451 | 1.00 | 105.66 | | O |
| ANISOU | 1835 | O | LEU | C | 252 | 16105 | 11122 | 12918 | −693 | −1805 | 1492 | O |
| ATOM | 1836 | CB | LEU | C | 252 | 79.940 | −6.112 | −9.403 | 1.00 | 106.33 | | C |
| ANISOU | 1836 | CB | LEU | C | 252 | 15658 | 11504 | 13238 | 286 | −2084 | 1207 | C |
| ATOM | 1837 | CG | LEU | C | 252 | 80.698 | −7.410 | −9.160 | 1.00 | 113.49 | | C |
| ANISOU | 1837 | CG | LEU | C | 252 | 16791 | 12139 | 14190 | 615 | −2437 | 1142 | C |
| ATOM | 1838 | CD1 | LEU | C | 252 | 81.579 | −7.745 | −10.325 | 1.00 | 114.70 | | C |
| ANISOU | 1838 | CD1 | LEU | C | 252 | 16694 | 12460 | 14427 | 1046 | −2465 | 902 | C |
| ATOM | 1839 | CD2 | LEU | C | 252 | 81.543 | −7.296 | −7.932 | 1.00 | 117.12 | | C |
| ANISOU | 1839 | CD2 | LEU | C | 252 | 17286 | 12583 | 14633 | 646 | −2631 | 1257 | C |
| ATOM | 1840 | N | TYR | C | 253 | 77.997 | −7.333 | −7.622 | 1.00 | 105.10 | | N |
| ANISOU | 1840 | N | TYR | C | 253 | 16317 | 10706 | 12910 | −304 | −2305 | 1522 | N |
| ATOM | 1841 | CA | TYR | C | 253 | 77.379 | −7.736 | −6.353 | 1.00 | 105.07 | | C |
| ANISOU | 1841 | CA | TYR | C | 253 | 16673 | 10457 | 12793 | −611 | −2440 | 1728 | C |
| ATOM | 1842 | C | TYR | C | 253 | 76.035 | −8.429 | −6.524 | 1.00 | 109.48 | | C |
| ANISOU | 1842 | C | TYR | C | 253 | 17542 | 10768 | 13287 | −889 | −2421 | 1766 | C |
| ATOM | 1843 | O | TYR | C | 253 | 75.858 | −9.493 | −5.944 | 1.00 | 111.33 | | O |
| ANISOU | 1843 | O | TYR | C | 253 | 18166 | 10650 | 13486 | −944 | −2667 | 1867 | O |
| ATOM | 1844 | CB | TYR | C | 253 | 77.279 | −6.593 | −5.323 | 1.00 | 103.71 | | C |
| ANISOU | 1844 | CB | TYR | C | 253 | 16362 | 10515 | 12528 | −886 | −2306 | 1870 | C |
| ATOM | 1845 | CG | TYR | C | 253 | 76.480 | −6.981 | −4.096 | 1.00 | 105.72 | | C |
| ANISOU | 1845 | CG | TYR | C | 253 | 16970 | 10570 | 12628 | −1236 | −2408 | 2076 | C |
| ATOM | 1846 | CD1 | TYR | C | 253 | 76.998 | −7.862 | −3.151 | 1.00 | 110.58 | | C |
| ANISOU | 1846 | CD1 | TYR | C | 253 | 17888 | 10927 | 13202 | −1163 | −2720 | 2206 | C |
| ATOM | 1847 | CD2 | TYR | C | 253 | 75.177 | −6.525 | −3.916 | 1.00 | 104.48 | | C |
| ANISOU | 1847 | CD2 | TYR | C | 253 | 16854 | 10490 | 12355 | −1635 | −2203 | 2139 | C |
| ATOM | 1848 | CE1 | TYR | C | 253 | 76.253 | −8.253 | −2.040 | 1.00 | 112.26 | | C |
| ANISOU | 1848 | CE1 | TYR | C | 253 | 18438 | 10967 | 13248 | −1506 | −2811 | 2417 | C |
| ATOM | 1849 | CE2 | TYR | C | 253 | 74.419 | −6.915 | −2.812 | 1.00 | 106.36 | | C |
| ANISOU | 1849 | CE2 | TYR | C | 253 | 17400 | 10585 | 12427 | −1977 | −2285 | 2326 | C |
| ATOM | 1850 | CZ | TYR | C | 253 | 74.963 | −7.778 | −1.876 | 1.00 | 116.55 | | C |
| ANISOU | 1850 | CZ | TYR | C | 253 | 18995 | 11622 | 13667 | −1924 | −2584 | 2476 | C |
| ATOM | 1851 | OH | TYR | C | 253 | 74.226 | −8.164 | −0.783 | 1.00 | 119.43 | | O |
| ANISOU | 1851 | OH | TYR | C | 253 | 19670 | 11863 | 13844 | −2282 | −2660 | 2682 | O |
| ATOM | 1852 | N | SER | C | 254 | 75.074 | −7.812 | −7.256 | 1.00 | 104.36 | | N |
| ANISOU | 1852 | N | SER | C | 254 | 16734 | 10304 | 12614 | −1083 | −2138 | 1698 | N |
| ATOM | 1853 | CA | SER | C | 254 | 73.736 | −8.357 | −7.542 | 1.00 | 104.31 | | C |
| ANISOU | 1853 | CA | SER | C | 254 | 16955 | 10133 | 12544 | −1361 | −2082 | 1699 | C |
| ATOM | 1854 | C | SER | C | 254 | 73.893 | −9.735 | −8.187 | 1.00 | 110.72 | | C |
| ANISOU | 1854 | C | SER | C | 254 | 18033 | 10594 | 13443 | −1140 | −2318 | 1599 | C |
| ATOM | 1855 | O | SER | C | 254 | 73.061 | −10.621 | −7.964 | 1.00 | 111.81 | | O |
| ANISOU | 1855 | O | SER | C | 254 | 18516 | 10436 | 13529 | −1374 | −2426 | 1664 | O |
| ATOM | 1856 | CB | SER | C | 254 | 72.959 | −7.418 | −8.464 | 1.00 | 104.73 | | C |
| ANISOU | 1856 | CB | SER | C | 254 | 16716 | 10490 | 12588 | −1457 | −1761 | 1579 | C |
| ATOM | 1857 | OG | SER | C | 254 | 71.567 | −7.687 | −8.434 | 1.00 | 112.02 | | O |
| ANISOU | 1857 | OG | SER | C | 254 | 17816 | 11332 | 13415 | −1799 | −1679 | 1597 | O |
| ATOM | 1858 | N | ILE | C | 255 | 75.015 | −9.910 | −8.936 | 1.00 | 107.86 | | N |
| ANISOU | 1858 | N | ILE | C | 255 | 17505 | 10269 | 13206 | −688 | −2412 | 1437 | N |
| ATOM | 1859 | CA | ILE | C | 255 | 75.464 | −11.140 | −9.591 | 1.00 | 110.47 | | C |
| ANISOU | 1859 | CA | ILE | C | 255 | 18029 | 10305 | 13640 | −358 | −2666 | 1291 | C |
| ATOM | 1860 | C | ILE | C | 255 | 75.751 | −12.172 | −8.505 | 1.00 | 118.15 | | C |
| ANISOU | 1860 | C | ILE | C | 255 | 19428 | 10866 | 14597 | −366 | −3018 | 1450 | C |
| ATOM | 1861 | O | ILE | C | 255 | 75.235 | −13.283 | −8.576 | 1.00 | 119.65 | | O |
| ANISOU | 1861 | O | ILE | C | 255 | 19993 | 10666 | 14801 | −426 | −3211 | 1452 | O |
| ATOM | 1862 | CB | ILE | C | 255 | 76.739 | −10.890 | −10.448 | 1.00 | 113.46 | | C |
| ANISOU | 1862 | CB | ILE | C | 255 | 18053 | 10930 | 14126 | 129 | −2661 | 1082 | C |
| ATOM | 1863 | CG1 | ILE | C | 255 | 76.577 | −9.690 | −11.399 | 1.00 | 110.49 | | C |
| ANISOU | 1863 | CG1 | ILE | C | 255 | 17244 | 10995 | 13742 | 110 | −2301 | 976 | C |
| ATOM | 1864 | CG2 | ILE | C | 255 | 77.157 | −12.162 | −11.201 | 1.00 | 117.44 | | C |
| ANISOU | 1864 | CG2 | ILE | C | 255 | 18737 | 11154 | 14731 | 503 | −2923 | 883 | C |
| ATOM | 1865 | CD1 | ILE | C | 255 | 77.885 | −9.163 | −11.983 | 1.00 | 118.27 | | C |
| ANISOU | 1865 | CD1 | ILE | C | 255 | 17825 | 12320 | 14792 | 479 | −2240 | 844 | C |
| ATOM | 1866 | N | TRP | C | 256 | 76.572 | −11.789 | −7.509 | 1.00 | 116.35 | | N |
| ANISOU | 1866 | N | TRP | C | 256 | 19150 | 10721 | 14338 | −310 | −3108 | 1583 | N |
| ATOM | 1867 | CA | TRP | C | 256 | 76.978 | −12.614 | −6.374 | 1.00 | 120.27 | | C |
| ANISOU | 1867 | CA | TRP | C | 256 | 20027 | 10874 | 14797 | −296 | −3446 | 1758 | C |
| ATOM | 1868 | C | TRP | C | 256 | 75.804 | −12.926 | −5.429 | 1.00 | 125.69 | | C |
| ANISOU | 1868 | C | TRP | C | 256 | 21090 | 11331 | 15335 | −810 | −3457 | 2006 | C |
| ATOM | 1869 | O | TRP | C | 256 | 75.735 | −14.039 | −4.911 | 1.00 | 127.99 | | O |
| ANISOU | 1869 | O | TRP | C | 256 | 21828 | 11194 | 15609 | −846 | −3751 | 2124 | O |
| ATOM | 1870 | CB | TRP | C | 256 | 78.134 | −11.934 | −5.635 | 1.00 | 119.10 | | C |
| ANISOU | 1870 | CB | TRP | C | 256 | 19654 | 10963 | 14637 | −116 | −3494 | 1814 | C |
| ATOM | 1871 | CG | TRP | C | 256 | 78.645 | −12.702 | −4.458 | 1.00 | 123.68 | | C |
| ANISOU | 1871 | CG | TRP | C | 256 | 20599 | 11231 | 15162 | −66 | −3846 | 1994 | C |
| ATOM | 1872 | CD1 | TRP | C | 256 | 79.577 | −13.698 | −4.467 | 1.00 | 130.35 | | C |
| ANISOU | 1872 | CD1 | TRP | C | 256 | 21637 | 11802 | 16090 | 350 | −4206 | 1925 | C |
| ATOM | 1873 | CD2 | TRP | C | 256 | 78.267 | −12.514 | −3.091 | 1.00 | 123.78 | | C |
| ANISOU | 1873 | CD2 | TRP | C | 256 | 20822 | 11197 | 15012 | −427 | −3882 | 2270 | C |
| ATOM | 1874 | CE2 | TRP | C | 256 | 79.000 | −13.445 | −2.322 | 1.00 | 131.81 | | C |
| ANISOU | 1874 | CE2 | TRP | C | 256 | 22179 | 11889 | 16014 | −217 | −4274 | 2378 | C |
| ATOM | 1875 | CE3 | TRP | C | 256 | 77.359 | −11.662 | −2.441 | 1.00 | 122.53 | | C |
| ANISOU | 1875 | CE3 | TRP | C | 256 | 20602 | 11248 | 14705 | −898 | −3629 | 2426 | C |
| ATOM | 1876 | NE1 | TRP | C | 256 | 79.790 | −14.157 | −3.188 | 1.00 | 132.47 | | N |
| ANISOU | 1876 | NE1 | TRP | C | 256 | 22249 | 11828 | 16258 | 268 | −4473 | 2158 | N |
| ATOM | 1877 | CZ2 | TRP | C | 256 | 78.859 | −13.544 | −0.933 | 1.00 | 132.60 | | C |
| ANISOU | 1877 | CZ2 | TRP | C | 256 | 22560 | 11877 | 15946 | −479 | −4410 | 2658 | C |
| ATOM | 1878 | CZ3 | TRP | C | 256 | 77.227 | −11.754 | −1.065 | 1.00 | 125.57 | | C |
| ANISOU | 1878 | CZ3 | TRP | C | 256 | 21244 | 11545 | 14924 | −1153 | −3757 | 2681 | C |
| ATOM | 1879 | CH2 | TRP | C | 256 | 77.966 | −12.690 | −0.326 | 1.00 | 130.14 | | C |
| ANISOU | 1879 | CH2 | TRP | C | 256 | 22165 | 11804 | 15480 | −954 | −4139 | 2805 | C |
| ATOM | 1880 | N | LYS | C | 257 | 74.881 | −11.954 | −5.229 | 1.00 | 120.79 | | N |
| ANISOU | 1880 | N | LYS | C | 257 | 20290 | 11001 | 14604 | −1204 | −3143 | 2080 | N |
| ATOM | 1881 | CA | LYS | C | 257 | 73.664 | −12.084 | −4.415 | 1.00 | 121.48 | | C |
| ANISOU | 1881 | CA | LYS | C | 257 | 20641 | 10991 | 14524 | −1726 | −3085 | 2284 | C |
| ATOM | 1882 | C | LYS | C | 257 | 72.674 | −13.084 | −5.062 | 1.00 | 129.75 | | C |
| ANISOU | 1882 | C | LYS | C | 257 | 21976 | 11733 | 15591 | −1900 | −3127 | 2233 | C |
| ATOM | 1883 | O | LYS | C | 257 | 71.869 | −13.693 | −4.352 | 1.00 | 131.14 | | O |
| ANISOU | 1883 | O | LYS | C | 257 | 22504 | 11679 | 15645 | −2287 | −3207 | 2420 | O |
| ATOM | 1884 | CB | LYS | C | 257 | 73.004 | −10.713 | −4.196 | 1.00 | 119.76 | | C |
| ANISOU | 1884 | CB | LYS | C | 257 | 20090 | 11215 | 14200 | −2020 | −2734 | 2303 | C |
| ATOM | 1885 | N | LYS | C | 258 | 72.756 | −13.255 | −6.409 | 1.00 | 127.70 | | N |
| ANISOU | 1885 | N | LYS | C | 258 | 21559 | 11487 | 15476 | −1622 | −3074 | 1978 | N |
| ATOM | 1886 | CA | LYS | C | 258 | 71.977 | −14.217 | −7.199 | 1.00 | 129.68 | | C |
| ANISOU | 1886 | CA | LYS | C | 258 | 22041 | 11454 | 15777 | −1703 | −3134 | 1869 | C |
| ATOM | 1887 | C | LYS | C | 258 | 72.736 | −15.573 | −7.269 | 1.00 | 139.50 | | C |
| ANISOU | 1887 | C | LYS | C | 258 | 23660 | 12205 | 17140 | −1384 | −3539 | 1837 | C |
| ATOM | 1888 | O | LYS | C | 258 | 72.105 | −16.632 | −7.176 | 1.00 | 141.80 | | O |
| ANISOU | 1888 | O | LYS | C | 258 | 24363 | 12087 | 17426 | −1591 | −3718 | 1897 | O |
| ATOM | 1889 | CB | LYS | C | 258 | 71.697 | −13.670 | −8.607 | 1.00 | 129.11 | | C |
| ANISOU | 1889 | CB | LYS | C | 258 | 21596 | 11676 | 15782 | −1546 | −2876 | 1598 | C |
| ATOM | 1890 | N | MET | C | 259 | 74.093 | −15.523 | −7.403 | 1.00 | 137.67 | | N |
| ANISOU | 1890 | N | MET | C | 259 | 23283 | 12016 | 17011 | −883 | −3692 | 1738 | N |
| ATOM | 1891 | CA | MET | C | 259 | 75.001 | −16.679 | −7.448 | 1.00 | 141.74 | | C |
| ANISOU | 1891 | CA | MET | C | 259 | 24096 | 12117 | 17642 | −482 | −4094 | 1674 | C |
| ATOM | 1892 | C | MET | C | 259 | 75.015 | −17.416 | −6.118 | 1.00 | 150.27 | | C |
| ANISOU | 1892 | C | MET | C | 259 | 25670 | 12793 | 18634 | −691 | −4391 | 1971 | C |
| ATOM | 1893 | O | MET | C | 259 | 75.169 | −18.635 | −6.116 | 1.00 | 154.07 | | O |
| ANISOU | 1893 | O | MET | C | 259 | 26582 | 12776 | 19182 | −580 | −4727 | 1975 | O |
| ATOM | 1894 | CB | MET | C | 259 | 76.430 | −16.264 | −7.831 | 1.00 | 143.63 | | C |
| ANISOU | 1894 | CB | MET | C | 259 | 23985 | 12609 | 17978 | 74 | −4147 | 1497 | C |
| ATOM | 1895 | N | GLU | C | 260 | 74.853 | −16.686 | −4.988 | 1.00 | 146.43 | | N |
| ANISOU | 1895 | N | GLU | C | 260 | 25136 | 12512 | 17989 | −996 | −4276 | 2220 | N |
| ATOM | 1896 | CA | GLU | C | 260 | 74.789 | −17.267 | −3.640 | 1.00 | 149.81 | | C |
| ANISOU | 1896 | CA | GLU | C | 260 | 26011 | 12626 | 18282 | −1252 | −4519 | 2538 | C |
| ATOM | 1897 | C | GLU | C | 260 | 73.466 | −18.021 | −3.462 | 1.00 | 157.32 | | C |
| ANISOU | 1897 | C | GLU | C | 260 | 27368 | 13261 | 19145 | −1773 | −4527 | 2693 | C |
| ATOM | 1898 | O | GLU | C | 260 | 73.426 | −19.041 | −2.769 | 1.00 | 160.68 | | O |
| ANISOU | 1898 | O | GLU | C | 260 | 28298 | 13240 | 19514 | −1916 | −4829 | 2913 | O |
| ATOM | 1899 | CB | GLU | C | 260 | 74.932 | −16.187 | −2.561 | 1.00 | 148.83 | | C |
| ANISOU | 1899 | CB | GLU | C | 260 | 25670 | 12880 | 17998 | −1444 | −4356 | 2725 | C |
| ATOM | 1900 | N | ARG | C | 261 | 72.392 | −17.523 | −4.113 | 1.00 | 152.48 | | N |
| ANISOU | 1900 | N | ARG | C | 261 | 26533 | 12886 | 18517 | −2056 | −4202 | 2575 | N |
| ATOM | 1901 | CA | ARG | C | 261 | 71.058 | −18.124 | −4.090 | 1.00 | 154.15 | | C |
| ANISOU | 1901 | CA | ARG | C | 261 | 27038 | 12884 | 18647 | −2567 | −4159 | 2669 | C |
| ATOM | 1902 | C | ARG | C | 261 | 71.020 | −19.402 | −4.950 | 1.00 | 162.61 | | C |
| ANISOU | 1902 | C | ARG | C | 261 | 28431 | 13465 | 19889 | −2388 | −4419 | 2510 | C |
| ATOM | 1903 | O | ARG | C | 261 | 70.697 | −20.474 | −4.427 | 1.00 | 166.32 | | O |
| ANISOU | 1903 | O | ARG | C | 261 | 29416 | 13450 | 20328 | −2629 | −4682 | 2692 | O |
| ATOM | 1904 | CB | ARG | C | 261 | 69.990 | −17.106 | −4.537 | 1.00 | 150.06 | | C |
| ANISOU | 1904 | CB | ARG | C | 261 | 26126 | 12846 | 18045 | −2893 | −3725 | 2574 | C |
| ATOM | 1905 | CG | ARG | C | 261 | 69.654 | −16.061 | −3.479 | 1.00 | 157.90 | | C |
| ANISOU | 1905 | CG | ARG | C | 261 | 26929 | 14234 | 18831 | −3218 | −3502 | 2769 | C |
| ATOM | 1906 | N | GLU | C | 262 | 71.385 | −19.290 | −6.253 | 1.00 | 158.41 | | N |
| ANISOU | 1906 | N | GLU | C | 262 | 27604 | 13053 | 19530 | −1960 | −4358 | 2174 | N |
| ATOM | 1907 | CA | GLU | C | 262 | 71.408 | −20.398 | −7.220 | 1.00 | 161.39 | | C |
| ANISOU | 1907 | CA | GLU | C | 262 | 28215 | 13025 | 20080 | −1718 | −4590 | 1953 | C |
| ATOM | 1908 | C | GLU | C | 262 | 72.650 | −21.307 | −7.060 | 1.00 | 169.92 | | C |
| ANISOU | 1908 | C | GLU | C | 262 | 29592 | 13683 | 21288 | −1214 | −5033 | 1929 | C |
| ATOM | 1909 | O | GLU | C | 262 | 72.835 | −21.897 | −5.994 | 1.00 | 172.15 | | O |
| ANISOU | 1909 | O | GLU | C | 262 | 30284 | 13624 | 21501 | −1339 | −5298 | 2203 | O |
| ATOM | 1910 | CB | GLU | C | 262 | 71.250 | −19.865 | −8.661 | 1.00 | 159.67 | | C |
| ANISOU | 1910 | CB | GLU | C | 262 | 27545 | 13161 | 19961 | −1484 | −4327 | 1600 | C |
| ATOM | 1911 | CG | GLU | C | 262 | 69.813 | −19.822 | −9.163 | 1.00 | 168.09 | | C |
| ANISOU | 1911 | CG | GLU | C | 262 | 28593 | 14299 | 20973 | −1937 | −4096 | 1541 | C |
| ATOM | 1912 | CD | GLU | C | 262 | 68.889 | −18.780 | −8.556 | 1.00 | 181.13 | | C |
| ANISOU | 1912 | CD | GLU | C | 262 | 30020 | 16369 | 22433 | −2440 | −3739 | 1712 | C |
| ATOM | 1913 | OE1 | GLU | C | 262 | 69.343 | −17.637 | −8.321 | 1.00 | 172.05 | | O |
| ANISOU | 1913 | OE1 | GLU | C | 262 | 28472 | 15669 | 21231 | −2320 | −3514 | 1722 | O |
| ATOM | 1914 | OE2 | GLU | C | 262 | 67.693 | −19.097 | −8.359 | 1.00 | 172.78 | | O |
| ANISOU | 1914 | OE2 | GLU | C | 262 | 29170 | 15201 | 21277 | −2953 | −3684 | 1816 | O |
| ATOM | 1915 | N | GLY | C | 263 | 73.465 | −21.417 | −8.112 | 1.00 | 167.84 | | N |
| ANISOU | 1915 | N | GLY | C | 263 | 29119 | 13461 | 21193 | −649 | −5113 | 1599 | N |
| ATOM | 1916 | CA | GLY | C | 263 | 74.677 | −22.229 | −8.124 | 1.00 | 172.02 | | C |
| ANISOU | 1916 | CA | GLY | C | 263 | 29855 | 13653 | 21851 | −91 | −5526 | 1498 | C |
| ATOM | 1917 | C | GLY | C | 263 | 75.958 | −21.416 | −8.116 | 1.00 | 175.84 | | C |
| ANISOU | 1917 | C | GLY | C | 263 | 29909 | 14554 | 22349 | 379 | −5478 | 1405 | C |
| ATOM | 1918 | O | GLY | C | 263 | 75.951 | −20.241 | −8.500 | 1.00 | 171.06 | | O |
| ANISOU | 1918 | O | GLY | C | 263 | 28790 | 14509 | 21697 | 350 | −5106 | 1335 | O |
| ATOM | 1919 | N | LYS | C | 264 | 77.080 | −22.055 | −7.701 | 1.00 | 176.10 | | N |
| ANISOU | 1919 | N | LYS | C | 264 | 30152 | 14311 | 22448 | 824 | −5869 | 1394 | N |
| ATOM | 1920 | CA | LYS | C | 264 | 78.422 | −21.449 | −7.617 | 1.00 | 174.85 | | C |
| ANISOU | 1920 | CA | LYS | C | 264 | 29618 | 14512 | 22306 | 1301 | −5892 | 1296 | C |
| ATOM | 1921 | C | LYS | C | 264 | 79.127 | −21.292 | −8.988 | 1.00 | 177.83 | | C |
| ANISOU | 1921 | C | LYS | C | 264 | 29530 | 15226 | 22811 | 1837 | −5801 | 878 | C |
| ATOM | 1922 | O | LYS | C | 264 | 80.280 | −20.846 | −9.042 | 1.00 | 177.22 | | O |
| ANISOU | 1922 | O | LYS | C | 264 | 29099 | 15487 | 22750 | 2239 | −5808 | 762 | O |
| ATOM | 1923 | CB | LYS | C | 264 | 79.305 | −22.221 | −6.618 | 1.00 | 182.08 | | C |
| ANISOU | 1923 | CB | LYS | C | 264 | 30938 | 15018 | 23227 | 1575 | −6360 | 1438 | C |
| ATOM | 1924 | N | THR | C | 265 | 78.419 | −21.642 | −10.088 | 1.00 | 174.26 | | N |
| ANISOU | 1924 | N | THR | C | 265 | 29063 | 14713 | 22434 | 1822 | −5709 | 652 | N |
| ATOM | 1925 | CA | THR | C | 265 | 78.888 | −21.533 | −11.472 | 1.00 | 173.22 | | C |
| ANISOU | 1925 | CA | THR | C | 265 | 28506 | 14914 | 22396 | 2280 | −5601 | 256 | C |
| ATOM | 1926 | C | THR | C | 265 | 78.986 | −20.063 | −11.885 | 1.00 | 171.89 | | C |
| ANISOU | 1926 | C | THR | C | 265 | 27724 | 15441 | 22146 | 2162 | −5119 | 235 | C |
| ATOM | 1927 | O | THR | C | 265 | 77.968 | −19.409 | −12.136 | 1.00 | 168.42 | | O |
| ANISOU | 1927 | O | THR | C | 265 | 27187 | 15167 | 21638 | 1724 | −4796 | 317 | O |
| ATOM | 1928 | CB | THR | C | 265 | 78.055 | −22.414 | −12.439 | 1.00 | 180.08 | | C |
| ANISOU | 1928 | CB | THR | C | 265 | 29603 | 15458 | 23362 | 2312 | −5701 | 20 | C |
| ATOM | 1929 | CG2 | THR | C | 265 | 76.551 | −22.390 | −12.142 | 1.00 | 176.89 | | C |
| ANISOU | 1929 | CG2 | THR | C | 265 | 29476 | 14839 | 22894 | 1654 | −5545 | 225 | C |
| ATOM | 1930 | OG1 | THR | C | 265 | 78.286 | −21.979 | −13.782 | 1.00 | 178.13 | | O |
| ANISOU | 1930 | OG1 | THR | C | 265 | 28862 | 15670 | 23151 | 2636 | −5476 | −328 | O |
| ATOM | 1931 | N | LEU | C | 266 | 80.226 | −19.545 | −11.923 | 1.00 | 167.95 | | N |
| ANISOU | 1931 | N | LEU | C | 266 | 26819 | 15342 | 21653 | 2550 | −5086 | 127 | N |
| ATOM | 1932 | CA | LEU | C | 266 | 80.522 | −18.161 | −12.294 | 1.00 | 163.70 | | C |
| ANISOU | 1932 | CA | LEU | C | 266 | 25699 | 15449 | 21050 | 2486 | −4668 | 105 | C |
| ATOM | 1933 | C | LEU | C | 266 | 80.143 | −17.894 | −13.753 | 1.00 | 166.80 | | C |
| ANISOU | 1933 | C | LEU | C | 266 | 25780 | 16132 | 21465 | 2579 | −4412 | −169 | C |
| ATOM | 1934 | O | LEU | C | 266 | 79.542 | −16.854 | −14.051 | 1.00 | 162.86 | | O |
| ANISOU | 1934 | O | LEU | C | 266 | 25000 | 15987 | 20893 | 2267 | −4030 | −102 | O |
| ATOM | 1935 | CB | LEU | C | 266 | 82.005 | −17.850 | −12.054 | 1.00 | 164.42 | | C |
| ANISOU | 1935 | CB | LEU | C | 266 | 25460 | 15861 | 21152 | 2898 | −4746 | 31 | C |
| ATOM | 1936 | N | GLU | C | 267 | 80.442 | −18.872 | −14.644 | 1.00 | 166.29 | | N |
| ANISOU | 1936 | N | GLU | C | 267 | 25791 | 15896 | 21495 | 3014 | −4643 | −480 | N |
| ATOM | 1937 | CA | GLU | C | 267 | 80.162 | −18.841 | −16.081 | 1.00 | 165.19 | | C |
| ANISOU | 1937 | CA | GLU | C | 267 | 25397 | 15995 | 21374 | 3186 | −4472 | −785 | C |
| ATOM | 1938 | C | GLU | C | 267 | 78.679 | −18.607 | −16.423 | 1.00 | 166.07 | | C |
| ANISOU | 1938 | C | GLU | C | 267 | 25626 | 16035 | 21440 | 2699 | −4233 | −705 | C |
| ATOM | 1939 | O | GLU | C | 267 | 78.391 | −18.115 | −17.516 | 1.00 | 163.80 | | O |
| ANISOU | 1939 | O | GLU | C | 267 | 25003 | 16120 | 21114 | 2718 | −3951 | −871 | O |
| ATOM | 1940 | CB | GLU | C | 267 | 80.681 | −20.118 | −16.752 | 1.00 | 171.23 | | C |
| ANISOU | 1940 | CB | GLU | C | 267 | 26337 | 16472 | 22249 | 3723 | −4845 | −1122 | C |
| ATOM | 1941 | N | GLN | C | 270 | 77.751 | −18.934 | −15.488 | 1.00 | 162.20 | | N |
| ANISOU | 1941 | N | GLN | C | 270 | 25594 | 15096 | 20937 | 2258 | −4341 | −449 | N |
| ATOM | 1942 | CA | GLN | C | 270 | 76.301 | −18.755 | −15.644 | 1.00 | 159.66 | | C |
| ANISOU | 1942 | CA | GLN | C | 270 | 25407 | 14694 | 20562 | 1752 | −4139 | −356 | C |
| ATOM | 1943 | C | GLN | C | 270 | 75.885 | −17.268 | −15.724 | 1.00 | 157.91 | | C |
| ANISOU | 1943 | C | GLN | C | 270 | 24780 | 14999 | 20221 | 1424 | −3686 | −218 | C |
| ATOM | 1944 | O | GLN | C | 270 | 76.642 | −16.397 | −15.281 | 1.00 | 156.15 | | O |
| ANISOU | 1944 | O | GLN | C | 270 | 24307 | 15070 | 19954 | 1449 | −3571 | −82 | O |
| ATOM | 1945 | CB | GLN | C | 270 | 75.548 | −19.477 | −14.517 | 1.00 | 162.99 | | C |
| ANISOU | 1945 | CB | GLN | C | 270 | 26399 | 14550 | 20980 | 1352 | −4373 | −93 | C |
| ATOM | 1946 | N | ALA | C | 271 | 74.679 | −17.002 | −16.304 | 1.00 | 151.09 | | N |
| ANISOU | 1946 | N | ALA | C | 271 | 23858 | 14242 | 19307 | 1132 | −3448 | −270 | N |
| ATOM | 1947 | CA | ALA | C | 271 | 73.999 | −15.708 | −16.536 | 1.00 | 145.83 | | C |
| ANISOU | 1947 | CA | ALA | C | 271 | 22858 | 14023 | 18529 | 815 | −3035 | −181 | C |
| ATOM | 1948 | C | ALA | C | 271 | 74.427 | −14.989 | −17.836 | 1.00 | 146.24 | | C |
| ANISOU | 1948 | C | ALA | C | 271 | 22402 | 14601 | 18560 | 1131 | −2781 | −391 | C |
| ATOM | 1949 | O | ALA | C | 271 | 75.505 | −15.260 | −18.374 | 1.00 | 146.92 | | O |
| ANISOU | 1949 | O | ALA | C | 271 | 22304 | 14821 | 18699 | 1581 | −2881 | −548 | O |
| ATOM | 1950 | CB | ALA | C | 271 | 74.114 | −14.775 | −15.333 | 1.00 | 144.34 | | C |
| ANISOU | 1950 | CB | ALA | C | 271 | 22653 | 13933 | 18258 | 480 | −2920 | 141 | C |
| ATOM | 1951 | N | ALA | C | 272 | 73.538 | −14.076 | −18.326 | 1.00 | 138.82 | | N |
| ANISOU | 1951 | N | ALA | C | 272 | 21244 | 13970 | 17531 | 878 | −2456 | −389 | N |
| ATOM | 1952 | CA | ALA | C | 272 | 73.582 | −13.275 | −19.565 | 1.00 | 135.75 | | C |
| ANISOU | 1952 | CA | ALA | C | 272 | 20417 | 14079 | 17083 | 1039 | −2163 | −540 | C |
| ATOM | 1953 | C | ALA | C | 272 | 74.910 | −12.562 | −19.876 | 1.00 | 137.61 | | C |
| ANISOU | 1953 | C | ALA | C | 272 | 20271 | 14690 | 17325 | 1416 | −2092 | −594 | C |
| ATOM | 1954 | O | ALA | C | 272 | 75.475 | −12.808 | −20.946 | 1.00 | 138.75 | | O |
| ANISOU | 1954 | O | ALA | C | 272 | 20233 | 15002 | 17482 | 1801 | −2117 | −849 | O |
| ATOM | 1955 | CB | ALA | C | 272 | 72.442 | −12.259 | −19.575 | 1.00 | 132.85 | | C |
| ANISOU | 1955 | CB | ALA | C | 272 | 19915 | 13956 | 16606 | 623 | −1845 | −382 | C |
| ATOM | 1956 | N | LEU | C | 273 | 75.361 | −11.638 | −18.982 | 1.00 | 130.68 | | N |
| ANISOU | 1956 | N | LEU | C | 273 | 19249 | 13981 | 16424 | 1287 | −1988 | −367 | N |
| ATOM | 1957 | CA | LEU | C | 273 | 76.579 | −10.801 | −19.061 | 1.00 | 128.90 | | C |
| ANISOU | 1957 | CA | LEU | C | 273 | 18649 | 14132 | 16196 | 1540 | −1895 | −367 | C |
| ATOM | 1958 | C | LEU | C | 273 | 76.542 | −9.737 | −20.192 | 1.00 | 127.03 | | C |
| ANISOU | 1958 | C | LEU | C | 273 | 17992 | 14398 | 15876 | 1590 | −1563 | −439 | C |
| ATOM | 1959 | O | LEU | C | 273 | 77.073 | −8.638 | −19.998 | 1.00 | 124.85 | | O |
| ANISOU | 1959 | O | LEU | C | 273 | 17432 | 14436 | 15568 | 1538 | −1381 | −311 | O |
| ATOM | 1960 | CB | LEU | C | 273 | 77.875 | −11.636 | −19.132 | 1.00 | 132.49 | | C |
| ANISOU | 1960 | CB | LEU | C | 273 | 19108 | 14499 | 16733 | 2009 | −2181 | −546 | C |
| ATOM | 1961 | N | LEU | C | 274 | 75.918 | −10.064 | −21.352 | 1.00 | 120.67 | | N |
| ANISOU | 1961 | N | LEU | C | 274 | 17159 | 13659 | 15031 | 1682 | −1495 | −637 | N |
| ATOM | 1962 | CA | LEU | C | 274 | 75.795 | −9.184 | −22.516 | 1.00 | 117.20 | | C |
| ANISOU | 1962 | CA | LEU | C | 274 | 16363 | 13676 | 14491 | 1744 | −1201 | −710 | C |
| ATOM | 1963 | C | LEU | C | 274 | 74.572 | −8.269 | −22.391 | 1.00 | 114.28 | | C |
| ANISOU | 1963 | C | LEU | C | 274 | 15996 | 13384 | 14042 | 1335 | −955 | −536 | C |
| ATOM | 1964 | O | LEU | C | 274 | 73.439 | −8.737 | −22.504 | 1.00 | 113.59 | | O |
| ANISOU | 1964 | O | LEU | C | 274 | 16125 | 13099 | 13936 | 1153 | −975 | −582 | O |
| ATOM | 1965 | CB | LEU | C | 274 | 75.742 | −10.014 | −23.809 | 1.00 | 119.29 | | C |
| ANISOU | 1965 | CB | LEU | C | 274 | 16597 | 13992 | 14735 | 2066 | −1266 | −1024 | C |
| ATOM | 1966 | N | ALA | C | 275 | 74.811 | −6.968 | −22.127 | 1.00 | 106.10 | | N |
| ANISOU | 1966 | N | ALA | C | 275 | 14720 | 12631 | 12962 | 1191 | −736 | −347 | N |
| ATOM | 1967 | CA | ALA | C | 275 | 73.777 | −5.941 | −21.956 | 1.00 | 101.96 | | C |
| ANISOU | 1967 | CA | ALA | C | 275 | 14169 | 12209 | 12364 | 842 | −509 | −184 | C |
| ATOM | 1968 | C | ALA | C | 275 | 74.234 | −4.583 | −22.482 | 1.00 | 101.82 | | C |
| ANISOU | 1968 | C | ALA | C | 275 | 13794 | 12614 | 12279 | 866 | −249 | −95 | C |
| ATOM | 1969 | O | ALA | C | 275 | 75.387 | −4.211 | −22.267 | 1.00 | 101.76 | | O |
| ANISOU | 1969 | O | ALA | C | 275 | 13601 | 12757 | 12307 | 989 | −249 | −39 | O |
| ATOM | 1970 | CB | ALA | C | 275 | 73.396 | −5.823 | −20.492 | 1.00 | 101.67 | | C |
| ANISOU | 1970 | CB | ALA | C | 275 | 14356 | 11909 | 12365 | 513 | −584 | 25 | C |
| ATOM | 1971 | N | VAL | C | 276 | 73.312 | −3.826 | −23.131 | 1.00 | 94.61 | | N |
| ANISOU | 1971 | N | VAL | C | 276 | 12796 | 11885 | 11266 | 733 | −36 | −74 | N |
| ATOM | 1972 | CA | VAL | C | 276 | 73.566 | −2.509 | −23.749 | 1.00 | 91.75 | | C |
| ANISOU | 1972 | CA | VAL | C | 276 | 12139 | 11898 | 10825 | 730 | 214 | 25 | C |
| ATOM | 1973 | C | VAL | C | 276 | 72.439 | −1.471 | −23.436 | 1.00 | 87.73 | | C |
| ANISOU | 1973 | C | VAL | C | 276 | 11675 | 11398 | 10261 | 408 | 372 | 181 | C |
| ATOM | 1974 | O | VAL | C | 276 | 71.267 | −1.835 | −23.434 | 1.00 | 86.11 | | O |
| ANISOU | 1974 | O | VAL | C | 276 | 11656 | 11038 | 10025 | 264 | 345 | 125 | O |
| ATOM | 1975 | CB | VAL | C | 276 | 73.831 | −2.706 | −25.276 | 1.00 | 97.83 | | C |
| ANISOU | 1975 | CB | VAL | C | 276 | 12709 | 12964 | 11498 | 1028 | 300 | −160 | C |
| ATOM | 1976 | CG1 | VAL | C | 276 | 72.648 | −3.354 | −25.997 | 1.00 | 97.67 | | C |
| ANISOU | 1976 | CG1 | VAL | C | 276 | 12830 | 12872 | 11408 | 1025 | 295 | −324 | C |
| ATOM | 1977 | CG2 | VAL | C | 276 | 74.261 | −1.418 | −25.968 | 1.00 | 97.37 | | C |
| ANISOU | 1977 | CG2 | VAL | C | 276 | 12343 | 13305 | 11347 | 1048 | 540 | −41 | C |
| ATOM | 1978 | N | ALA | C | 277 | 72.801 | −0.198 | −23.147 | 1.00 | 80.16 | | N |
| ANISOU | 1978 | N | ALA | C | 277 | 10549 | 10615 | 9293 | 295 | 522 | 365 | N |
| ATOM | 1979 | CA | ALA | C | 277 | 71.821 | 0.863 | −22.841 | 1.00 | 76.83 | | C |
| ANISOU | 1979 | CA | ALA | C | 277 | 10165 | 10201 | 8826 | 29 | 651 | 497 | C |
| ATOM | 1980 | C | ALA | C | 277 | 71.733 | 1.900 | −23.951 | 1.00 | 77.75 | | C |
| ANISOU | 1980 | C | ALA | C | 277 | 10075 | 10620 | 8845 | 70 | 865 | 560 | C |
| ATOM | 1981 | O | ALA | C | 277 | 72.725 | 2.160 | −24.608 | 1.00 | 78.45 | | O |
| ANISOU | 1981 | O | ALA | C | 277 | 9957 | 10919 | 8931 | 189 | 940 | 619 | O |
| ATOM | 1982 | CB | ALA | C | 277 | 72.160 | 1.541 | −21.527 | 1.00 | 76.58 | | C |
| ANISOU | 1982 | CB | ALA | C | 277 | 10189 | 10031 | 8878 | −190 | 605 | 666 | C |
| ATOM | 1983 | N | VAL | C | 278 | 70.549 | 2.483 | −24.172 | 1.00 | 71.37 | | N |
| ANISOU | 1983 | N | VAL | C | 278 | 9324 | 9843 | 7949 | −36 | 956 | 554 | N |
| ATOM | 1984 | CA | VAL | C | 278 | 70.303 | 3.487 | −25.223 | 1.00 | 69.94 | | C |
| ANISOU | 1984 | CA | VAL | C | 278 | 8997 | 9919 | 7657 | 4 | 1139 | 620 | C |
| ATOM | 1985 | C | VAL | C | 278 | 69.653 | 4.725 | −24.591 | 1.00 | 72.34 | | C |
| ANISOU | 1985 | C | VAL | C | 278 | 9350 | 10172 | 7964 | −229 | 1205 | 773 | C |
| ATOM | 1986 | O | VAL | C | 278 | 68.513 | 4.654 | −24.099 | 1.00 | 70.85 | | O |
| ANISOU | 1986 | O | VAL | C | 278 | 9309 | 9855 | 7758 | −371 | 1166 | 718 | O |
| ATOM | 1987 | CB | VAL | C | 278 | 69.451 | 2.917 | −26.398 | 1.00 | 73.21 | | C |
| ANISOU | 1987 | CB | VAL | C | 278 | 9419 | 10456 | 7941 | 153 | 1176 | 443 | C |
| ATOM | 1988 | CG1 | VAL | C | 278 | 69.045 | 4.010 | −27.378 | 1.00 | 72.30 | | C |
| ANISOU | 1988 | CG1 | VAL | C | 278 | 9185 | 10593 | 7693 | 189 | 1350 | 529 | C |
| ATOM | 1989 | CG2 | VAL | C | 278 | 70.181 | 1.794 | −27.122 | 1.00 | 74.82 | | C |
| ANISOU | 1989 | CG2 | VAL | C | 278 | 9557 | 10732 | 8137 | 413 | 1108 | 272 | C |
| ATOM | 1990 | N | ILE | C | 279 | 70.380 | 5.851 | −24.578 | 1.00 | 68.20 | | N |
| ANISOU | 1990 | N | ILE | C | 279 | 8698 | 9755 | 7461 | −270 | 1300 | 953 | N |
| ATOM | 1991 | CA | ILE | C | 279 | 69.814 | 7.058 | −23.982 | 1.00 | 66.17 | | C |
| ANISOU | 1991 | CA | ILE | C | 279 | 8492 | 9433 | 7217 | −464 | 1344 | 1083 | C |
| ATOM | 1992 | C | ILE | C | 279 | 69.372 | 7.963 | −25.088 | 1.00 | 71.76 | | C |
| ANISOU | 1992 | C | ILE | C | 279 | 9132 | 10330 | 7805 | −394 | 1481 | 1144 | C |
| ATOM | 1993 | O | ILE | C | 279 | 70.143 | 8.231 | −26.012 | 1.00 | 73.40 | | O |
| ANISOU | 1993 | O | ILE | C | 279 | 9191 | 10735 | 7963 | −276 | 1575 | 1218 | O |
| ATOM | 1994 | CB | ILE | C | 279 | 70.694 | 7.759 | −22.912 | 1.00 | 68.22 | | C |
| ANISOU | 1994 | CB | ILE | C | 279 | 8718 | 9596 | 7606 | −618 | 1312 | 1238 | C |
| ATOM | 1995 | CG1 | ILE | C | 279 | 71.454 | 6.752 | −21.990 | 1.00 | 68.73 | | C |
| ANISOU | 1995 | CG1 | ILE | C | 279 | 8821 | 9519 | 7776 | −625 | 1166 | 1186 | C |
| ATOM | 1996 | CG2 | ILE | C | 279 | 69.881 | 8.774 | −22.106 | 1.00 | 66.86 | | C |
| ANISOU | 1996 | CG2 | ILE | C | 279 | 8644 | 9309 | 7452 | −813 | 1313 | 1309 | C |
| ATOM | 1997 | CD1 | ILE | C | 279 | 70.611 | 5.693 | −21.145 | 1.00 | 75.67 | | C |
| ANISOU | 1997 | CD1 | ILE | C | 279 | 9911 | 10180 | 8661 | −704 | 1026 | 1058 | C |
| ATOM | 1998 | N | LEU | C | 280 | 68.106 | 8.388 | −25.020 | 1.00 | 67.45 | | N |
| ANISOU | 1998 | N | LEU | C | 280 | 8690 | 9743 | 7195 | −457 | 1490 | 1104 | N |
| ATOM | 1999 | CA | LEU | C | 280 | 67.483 | 9.227 | −26.039 | 1.00 | 67.51 | | C |
| ANISOU | 1999 | CA | LEU | C | 280 | 8670 | 9909 | 7072 | −372 | 1593 | 1147 | C |
| ATOM | 2000 | C | LEU | C | 280 | 67.289 | 10.660 | −25.556 | 1.00 | 72.34 | | C |
| ANISOU | 2000 | C | LEU | C | 280 | 9324 | 10443 | 7719 | −504 | 1614 | 1307 | C |
| ATOM | 2001 | O | LEU | C | 280 | 66.907 | 10.860 | −24.405 | 1.00 | 70.73 | | O |
| ANISOU | 2001 | O | LEU | C | 280 | 9211 | 10072 | 7593 | −655 | 1538 | 1281 | O |
| ATOM | 2002 | CB | LEU | C | 280 | 66.131 | 8.604 | −26.508 | 1.00 | 66.44 | | C |
| ANISOU | 2002 | CB | LEU | C | 280 | 8606 | 9822 | 6817 | −294 | 1573 | 944 | C |
| ATOM | 2003 | CG | LEU | C | 280 | 66.163 | 7.130 | −26.962 | 1.00 | 69.88 | | C |
| ANISOU | 2003 | CG | LEU | C | 280 | 9035 | 10288 | 7228 | −178 | 1523 | 750 | C |
| ATOM | 2004 | CD1 | LEU | C | 280 | 64.791 | 6.568 | −27.087 | 1.00 | 68.69 | | C |
| ANISOU | 2004 | CD1 | LEU | C | 280 | 8973 | 10132 | 6995 | −188 | 1480 | 549 | C |
| ATOM | 2005 | CD2 | LEU | C | 280 | 66.959 | 6.942 | −28.236 | 1.00 | 71.66 | | C |
| ANISOU | 2005 | CD2 | LEU | C | 280 | 9120 | 10737 | 7372 | 39 | 1601 | 758 | C |
| ATOM | 2006 | N | ASP | C | 281 | 67.579 | 11.659 | −26.417 | 1.00 | 70.95 | | N |
| ANISOU | 2006 | N | ASP | C | 281 | 9090 | 10386 | 7483 | −451 | 1710 | 1472 | N |
| ATOM | 2007 | CA | ASP | C | 281 | 67.343 | 13.048 | −26.048 | 1.00 | 71.30 | | C |
| ANISOU | 2007 | CA | ASP | C | 281 | 9206 | 10324 | 7563 | −560 | 1710 | 1618 | C |
| ATOM | 2008 | C | ASP | C | 281 | 66.222 | 13.644 | −26.879 | 1.00 | 74.22 | | C |
| ANISOU | 2008 | C | ASP | C | 281 | 9639 | 10780 | 7781 | −436 | 1740 | 1600 | C |
| ATOM | 2009 | O | ASP | C | 281 | 66.466 | 14.171 | −27.965 | 1.00 | 74.35 | | O |
| ANISOU | 2009 | O | ASP | C | 281 | 9613 | 10943 | 7693 | −337 | 1827 | 1731 | O |
| ATOM | 2010 | CB | ASP | C | 281 | 68.608 | 13.926 | −26.062 | 1.00 | 75.42 | | C |
| ANISOU | 2010 | CB | ASP | C | 281 | 9651 | 10836 | 8167 | −661 | 1762 | 1856 | C |
| ATOM | 2011 | CG | ASP | C | 281 | 68.326 | 15.395 | −25.722 | 1.00 | 92.34 | | C |
| ANISOU | 2011 | CG | ASP | C | 281 | 11899 | 12831 | 10353 | −769 | 1742 | 2003 | C |
| ATOM | 2012 | OD1 | ASP | C | 281 | 68.312 | 15.735 | −24.515 | 1.00 | 90.49 | | O |
| ANISOU | 2012 | OD1 | ASP | C | 281 | 11729 | 12404 | 10249 | −913 | 1654 | 1984 | O |
| ATOM | 2013 | OD2 | ASP | C | 281 | 68.101 | 16.204 | −26.674 | 1.00 | 104.92 | | O |
| ANISOU | 2013 | OD2 | ASP | C | 281 | 13523 | 14499 | 11842 | −699 | 1804 | 2132 | O |
| ATOM | 2014 | N | PRO | C | 282 | 64.975 | 13.617 | −26.382 | 1.00 | 70.38 | | N |
| ANISOU | 2014 | N | PRO | C | 282 | 9250 | 10226 | 7266 | −439 | 1668 | 1440 | N |
| ATOM | 2015 | CA | PRO | C | 282 | 63.903 | 14.275 | −27.132 | 1.00 | 71.27 | | C |
| ANISOU | 2015 | CA | PRO | C | 282 | 9418 | 10428 | 7233 | −299 | 1679 | 1413 | C |
| ATOM | 2016 | C | PRO | C | 282 | 64.253 | 15.758 | −27.261 | 1.00 | 76.59 | | C |
| ANISOU | 2016 | C | PRO | C | 282 | 10155 | 11011 | 7933 | −328 | 1688 | 1647 | C |
| ATOM | 2017 | O | PRO | C | 282 | 64.696 | 16.379 | −26.293 | 1.00 | 77.50 | | O |
| ANISOU | 2017 | O | PRO | C | 282 | 10307 | 10942 | 8197 | −489 | 1644 | 1741 | O |
| ATOM | 2018 | CB | PRO | C | 282 | 62.663 | 14.050 | −26.252 | 1.00 | 71.93 | | C |
| ANISOU | 2018 | CB | PRO | C | 282 | 9567 | 10452 | 7310 | −341 | 1588 | 1188 | C |
| ATOM | 2019 | CG | PRO | C | 282 | 63.203 | 13.835 | −24.879 | 1.00 | 75.44 | | C |
| ANISOU | 2019 | CG | PRO | C | 282 | 10028 | 10718 | 7916 | −546 | 1529 | 1191 | C |
| ATOM | 2020 | CD | PRO | C | 282 | 64.487 | 13.093 | −25.091 | 1.00 | 71.06 | | C |
| ANISOU | 2020 | CD | PRO | C | 282 | 9393 | 10170 | 7435 | −574 | 1573 | 1288 | C |
| ATOM | 2021 | N | LYS | C | 283 | 64.143 | 16.309 | −28.448 | 1.00 | 72.90 | | N |
| ANISOU | 2021 | N | LYS | C | 283 | 9709 | 10665 | 7325 | −183 | 1739 | 1750 | N |
| ATOM | 2022 | CA | LYS | C | 283 | 64.457 | 17.715 | −28.556 | 1.00 | 74.01 | | C |
| ANISOU | 2022 | CA | LYS | C | 283 | 9945 | 10678 | 7497 | −230 | 1730 | 1988 | C |
| ATOM | 2023 | C | LYS | C | 283 | 63.260 | 18.566 | −28.141 | 1.00 | 76.80 | | C |
| ANISOU | 2023 | C | LYS | C | 283 | 10442 | 10892 | 7848 | −178 | 1611 | 1893 | C |
| ATOM | 2024 | O | LYS | C | 283 | 62.166 | 18.038 | −27.914 | 1.00 | 76.07 | | O |
| ANISOU | 2024 | O | LYS | C | 283 | 10344 | 10857 | 7702 | −103 | 1557 | 1641 | O |
| ATOM | 2025 | CB | LYS | C | 283 | 64.939 | 18.054 | −29.956 | 1.00 | 79.36 | | C |
| ANISOU | 2025 | CB | LYS | C | 283 | 10604 | 11529 | 8021 | −126 | 1831 | 2194 | C |
| ATOM | 2026 | N | ALA | C | 284 | 63.486 | 19.879 | −28.020 | 1.00 | 72.65 | | N |
| ANISOU | 2026 | N | ALA | C | 284 | 10040 | 10183 | 7380 | −225 | 1563 | 2087 | N |
| ATOM | 2027 | CA | ALA | C | 284 | 62.525 | 20.898 | −27.614 | 1.00 | 71.75 | | C |
| ANISOU | 2027 | CA | ALA | C | 284 | 10078 | 9902 | 7283 | −160 | 1429 | 2022 | C |
| ATOM | 2028 | C | ALA | C | 284 | 61.095 | 20.755 | −28.186 | 1.00 | 72.89 | | C |
| ANISOU | 2028 | C | ALA | C | 284 | 10253 | 10198 | 7245 | 89 | 1378 | 1816 | C |
| ATOM | 2029 | O | ALA | C | 284 | 60.907 | 20.951 | −29.384 | 1.00 | 74.10 | | O |
| ANISOU | 2029 | O | ALA | C | 284 | 10445 | 10469 | 7240 | 254 | 1410 | 1920 | O |
| ATOM | 2030 | CB | ALA | C | 284 | 63.090 | 22.266 | −27.960 | 1.00 | 74.63 | | C |
| ANISOU | 2030 | CB | ALA | C | 284 | 10590 | 10068 | 7700 | −213 | 1398 | 2314 | C |
| ATOM | 2031 | N | ALA | C | 285 | 60.091 | 20.430 | −27.329 | 1.00 | 65.85 | | N |
| ANISOU | 2031 | N | ALA | C | 285 | 9333 | 9326 | 6360 | 112 | 1298 | 1525 | N |
| ATOM | 2032 | CA | ALA | C | 285 | 58.677 | 20.358 | −27.746 | 1.00 | 64.79 | | C |
| ANISOU | 2032 | CA | ALA | C | 285 | 9202 | 9356 | 6057 | 336 | 1238 | 1295 | C |
| ATOM | 2033 | C | ALA | C | 285 | 57.902 | 21.651 | −27.320 | 1.00 | 67.40 | | C |
| ANISOU | 2033 | C | ALA | C | 285 | 9675 | 9528 | 6404 | 451 | 1080 | 1244 | C |
| ATOM | 2034 | O | ALA | C | 285 | 58.311 | 22.273 | −26.331 | 1.00 | 67.10 | | O |
| ANISOU | 2034 | O | ALA | C | 285 | 9700 | 9264 | 6529 | 312 | 1014 | 1290 | O |
| ATOM | 2035 | CB | ALA | C | 285 | 58.020 | 19.120 | −27.195 | 1.00 | 63.72 | | C |
| ANISOU | 2035 | CB | ALA | C | 285 | 8931 | 9389 | 5890 | 285 | 1254 | 998 | C |
| ATOM | 2036 | N | PRO | C | 286 | 56.838 | 22.102 | −28.059 | 1.00 | 62.69 | | N |
| ANISOU | 2036 | N | PRO | C | 286 | 9136 | 9042 | 5642 | 720 | 1003 | 1146 | N |
| ATOM | 2037 | CA | PRO | C | 286 | 56.182 | 23.393 | −27.742 | 1.00 | 63.77 | | C |
| ANISOU | 2037 | CA | PRO | C | 286 | 9427 | 9007 | 5796 | 871 | 830 | 1105 | C |
| ATOM | 2038 | C | PRO | C | 286 | 55.233 | 23.431 | −26.546 | 1.00 | 68.45 | | C |
| ANISOU | 2038 | C | PRO | C | 286 | 9960 | 9628 | 6421 | 882 | 721 | 771 | C |
| ATOM | 2039 | O | PRO | C | 286 | 54.761 | 24.512 | −26.143 | 1.00 | 67.71 | | O |
| ANISOU | 2039 | O | PRO | C | 286 | 9987 | 9376 | 6365 | 1005 | 562 | 711 | O |
| ATOM | 2040 | CB | PRO | C | 286 | 55.411 | 23.729 | −29.025 | 1.00 | 66.72 | | C |
| ANISOU | 2040 | CB | PRO | C | 286 | 9868 | 9525 | 5960 | 1176 | 785 | 1113 | C |
| ATOM | 2041 | CG | PRO | C | 286 | 55.772 | 22.716 | −29.986 | 1.00 | 70.61 | | C |
| ANISOU | 2041 | CG | PRO | C | 286 | 10242 | 10254 | 6334 | 1163 | 943 | 1175 | C |
| ATOM | 2042 | CD | PRO | C | 286 | 56.234 | 21.517 | −29.262 | 1.00 | 63.90 | | C |
| ANISOU | 2042 | CD | PRO | C | 286 | 9223 | 9473 | 5583 | 920 | 1055 | 1070 | C |
| ATOM | 2043 | N | THR | C | 287 | 54.917 | 22.246 | −26.012 | 1.00 | 65.41 | | N |
| ANISOU | 2043 | N | THR | C | 287 | 9391 | 9456 | 6006 | 762 | 797 | 544 | N |
| ATOM | 2044 | CA | THR | C | 287 | 54.040 | 22.101 | −24.870 | 1.00 | 65.12 | | C |
| ANISOU | 2044 | CA | THR | C | 287 | 9262 | 9515 | 5967 | 728 | 725 | 226 | C |
| ATOM | 2045 | C | THR | C | 287 | 54.402 | 20.827 | −24.075 | 1.00 | 67.69 | | C |
| ANISOU | 2045 | C | THR | C | 287 | 9447 | 9921 | 6353 | 438 | 836 | 155 | C |
| ATOM | 2046 | O | THR | C | 287 | 55.085 | 19.946 | −24.587 | 1.00 | 66.11 | | O |
| ANISOU | 2046 | O | THR | C | 287 | 9204 | 9750 | 6163 | 332 | 955 | 289 | O |
| ATOM | 2047 | CB | THR | C | 287 | 52.567 | 22.236 | −25.300 | 1.00 | 73.97 | | C |
| ANISOU | 2047 | CB | THR | C | 287 | 10321 | 10892 | 6890 | 1003 | 642 | −61 | C |
| ATOM | 2048 | CG2 | THR | C | 287 | 52.090 | 21.133 | −26.210 | 1.00 | 62.78 | | C |
| ANISOU | 2048 | CG2 | THR | C | 287 | 8767 | 9774 | 5313 | 1042 | 745 | −167 | C |
| ATOM | 2049 | OG1 | THR | C | 287 | 51.765 | 22.337 | −24.126 | 1.00 | 88.03 | | O |
| ANISOU | 2049 | OG1 | THR | C | 287 | 12017 | 12760 | 8668 | 980 | 555 | −360 | O |
| ATOM | 2050 | N | ARG | C | 288 | 53.978 | 20.766 | −22.811 | 1.00 | 65.39 | | N |
| ANISOU | 2050 | N | ARG | C | 288 | 9093 | 9655 | 6096 | 316 | 784 | −49 | N |
| ATOM | 2051 | CA | ARG | C | 288 | 54.235 | 19.626 | −21.924 | 1.00 | 64.41 | | C |
| ANISOU | 2051 | CA | ARG | C | 288 | 8864 | 9594 | 6017 | 35 | 864 | −114 | C |
| ATOM | 2052 | C | ARG | C | 288 | 53.415 | 18.431 | −22.387 | 1.00 | 66.86 | | C |
| ANISOU | 2052 | C | ARG | C | 288 | 9039 | 10191 | 6171 | 23 | 937 | −306 | C |
| ATOM | 2053 | O | ARG | C | 288 | 53.751 | 17.285 | −22.080 | 1.00 | 65.20 | | O |
| ANISOU | 2053 | O | ARG | C | 288 | 8773 | 10013 | 5988 | −192 | 1016 | −301 | O |
| ATOM | 2054 | CB | ARG | C | 288 | 53.911 | 19.979 | −20.446 | 1.00 | 65.94 | | C |
| ANISOU | 2054 | CB | ARG | C | 288 | 9029 | 9770 | 6256 | −85 | 782 | −284 | C |
| ATOM | 2055 | N | GLU | C | 289 | 52.341 | 18.713 | −23.123 | 1.00 | 64.07 | | N |
| ANISOU | 2055 | N | GLU | C | 289 | 8644 | 10039 | 5661 | 260 | 895 | −479 | N |
| ATOM | 2056 | CA | GLU | C | 289 | 51.441 | 17.703 | −23.646 | 1.00 | 64.69 | | C |
| ANISOU | 2056 | CA | GLU | C | 289 | 8587 | 10412 | 5580 | 271 | 947 | −691 | C |
| ATOM | 2057 | C | GLU | C | 289 | 52.051 | 17.009 | −24.853 | 1.00 | 69.54 | | C |
| ANISOU | 2057 | C | GLU | C | 289 | 9222 | 11019 | 6182 | 308 | 1041 | −523 | C |
| ATOM | 2058 | O | GLU | C | 289 | 51.950 | 15.783 | −24.928 | 1.00 | 68.14 | | O |
| ANISOU | 2058 | O | GLU | C | 289 | 8964 | 10950 | 5978 | 156 | 1113 | −605 | O |
| ATOM | 2059 | CB | GLU | C | 289 | 50.028 | 18.254 | −23.929 | 1.00 | 67.87 | | C |
| ANISOU | 2059 | CB | GLU | C | 289 | 8908 | 11071 | 5808 | 519 | 857 | −976 | C |
| ATOM | 2060 | CG | GLU | C | 289 | 49.768 | 19.737 | −23.634 | 1.00 | 83.81 | | C |
| ANISOU | 2060 | CG | GLU | C | 289 | 11015 | 12984 | 7845 | 739 | 713 | −1002 | C |
| ATOM | 2061 | CD | GLU | C | 289 | 49.867 | 20.301 | −22.221 | 1.00 | 107.64 | | C |
| ANISOU | 2061 | CD | GLU | C | 289 | 14047 | 15887 | 10966 | 611 | 645 | −1065 | C |
| ATOM | 2062 | OE1 | GLU | C | 289 | 50.264 | 21.484 | −22.087 | 1.00 | 76.99 | | O |
| ANISOU | 2062 | OE1 | GLU | C | 289 | 10315 | 11747 | 7190 | 729 | 546 | −929 | O |
| ATOM | 2063 | OE2 | GLU | C | 289 | 49.527 | 19.580 | −21.253 | 1.00 | 115.58 | | O |
| ANISOU | 2063 | OE2 | GLU | C | 289 | 14918 | 17062 | 11936 | 390 | 684 | −1252 | O |
| ATOM | 2064 | N | SER | C | 290 | 52.736 | 17.786 | −25.760 | 1.00 | 67.90 | | N |
| ANISOU | 2064 | N | SER | C | 290 | 9130 | 10676 | 5996 | 493 | 1037 | −280 | N |
| ATOM | 2065 | CA | SER | C | 290 | 53.444 | 17.315 | −26.975 | 1.00 | 67.67 | | C |
| ANISOU | 2065 | CA | SER | C | 290 | 9118 | 10656 | 5937 | 556 | 1128 | −93 | C |
| ATOM | 2066 | C | SER | C | 290 | 54.772 | 16.624 | −26.619 | 1.00 | 70.59 | | C |
| ANISOU | 2066 | C | SER | C | 290 | 9500 | 10859 | 6463 | 322 | 1216 | 104 | C |
| ATOM | 2067 | O | SER | C | 290 | 55.157 | 15.642 | −27.266 | 1.00 | 69.50 | | O |
| ANISOU | 2067 | O | SER | C | 290 | 9309 | 10803 | 6297 | 297 | 1297 | 119 | O |
| ATOM | 2068 | CB | SER | C | 290 | 53.674 | 18.455 | −27.961 | 1.00 | 71.55 | | C |
| ANISOU | 2068 | CB | SER | C | 290 | 9732 | 11073 | 6381 | 799 | 1091 | 115 | C |
| ATOM | 2069 | OG | SER | C | 290 | 54.581 | 19.427 | −27.476 | 1.00 | 78.66 | | O |
| ANISOU | 2069 | OG | SER | C | 290 | 10764 | 11685 | 7436 | 728 | 1057 | 358 | O |
| ATOM | 2070 | N | GLN | C | 291 | 55.458 | 17.145 | −25.570 | 1.00 | 66.54 | | N |
| ANISOU | 2070 | N | GLN | C | 291 | 9051 | 10118 | 6113 | 164 | 1187 | 231 | N |
| ATOM | 2071 | CA | GLN | C | 291 | 56.639 | 16.552 | −24.953 | 1.00 | 65.40 | | C |
| ANISOU | 2071 | CA | GLN | C | 291 | 8906 | 9820 | 6123 | −64 | 1243 | 380 | C |
| ATOM | 2072 | C | GLN | C | 291 | 55.973 | 15.498 | −24.094 | 1.00 | 70.62 | | C |
| ANISOU | 2072 | C | GLN | C | 291 | 9488 | 10585 | 6761 | −233 | 1237 | 140 | C |
| ATOM | 2073 | O | GLN | C | 291 | 54.776 | 15.631 | −23.822 | 1.00 | 70.78 | | O |
| ANISOU | 2073 | O | GLN | C | 291 | 9463 | 10751 | 6679 | −192 | 1184 | −88 | O |
| ATOM | 2074 | CB | GLN | C | 291 | 57.369 | 17.590 | −24.083 | 1.00 | 66.73 | | C |
| ANISOU | 2074 | CB | GLN | C | 291 | 9162 | 9742 | 6451 | −161 | 1191 | 547 | C |
| ATOM | 2075 | CG | GLN | C | 291 | 58.751 | 17.173 | −23.588 | 1.00 | 84.31 | | C |
| ANISOU | 2075 | CG | GLN | C | 291 | 11385 | 11813 | 8835 | −362 | 1243 | 738 | C |
| ATOM | 2076 | CD | GLN | C | 291 | 59.786 | 17.060 | −24.681 | 1.00 | 108.44 | | C |
| ANISOU | 2076 | CD | GLN | C | 291 | 14442 | 14858 | 11904 | −304 | 1330 | 978 | C |
| ATOM | 2077 | NE2 | GLN | C | 291 | 60.433 | 18.176 | −25.006 | 1.00 | 100.77 | | N |
| ANISOU | 2077 | NE2 | GLN | C | 291 | 13551 | 13751 | 10987 | −272 | 1318 | 1197 | N |
| ATOM | 2078 | OE1 | GLN | C | 291 | 60.050 | 15.968 | −25.205 | 1.00 | 104.94 | | O |
| ANISOU | 2078 | OE1 | GLN | C | 291 | 13926 | 14525 | 11423 | −301 | 1404 | 969 | O |
| ATOM | 2079 | N | ALA | C | 292 | 56.673 | 14.422 | −23.722 | 1.00 | 67.88 | | N |
| ANISOU | 2079 | N | ALA | C | 292 | 9120 | 10184 | 6488 | −413 | 1283 | 179 | N |
| ATOM | 2080 | CA | ALA | C | 292 | 56.052 | 13.300 | −22.980 | 1.00 | 67.83 | | C |
| ANISOU | 2080 | CA | ALA | C | 292 | 9063 | 10262 | 6447 | −600 | 1275 | −23 | C |
| ATOM | 2081 | C | ALA | C | 292 | 55.292 | 12.411 | −23.981 | 1.00 | 72.69 | | C |
| ANISOU | 2081 | C | ALA | C | 292 | 9615 | 11081 | 6924 | −506 | 1308 | −191 | C |
| ATOM | 2082 | O | ALA | C | 292 | 55.473 | 11.185 | −23.940 | 1.00 | 73.84 | | O |
| ANISOU | 2082 | O | ALA | C | 292 | 9752 | 11217 | 7085 | −632 | 1329 | −237 | O |
| ATOM | 2083 | CB | ALA | C | 292 | 55.134 | 13.775 | −21.849 | 1.00 | 68.50 | | C |
| ANISOU | 2083 | CB | ALA | C | 292 | 9125 | 10410 | 6493 | −696 | 1211 | −195 | C |
| ATOM | 2084 | N | LEU | C | 293 | 54.511 | 13.025 | −24.933 | 1.00 | 66.59 | | N |
| ANISOU | 2084 | N | LEU | C | 293 | 8807 | 10478 | 6018 | −270 | 1301 | −278 | N |
| ATOM | 2085 | CA | LEU | C | 293 | 53.883 | 12.288 | −26.027 | 1.00 | 64.94 | | C |
| ANISOU | 2085 | CA | LEU | C | 293 | 8529 | 10473 | 5672 | −148 | 1329 | −430 | C |
| ATOM | 2086 | C | LEU | C | 293 | 55.057 | 11.767 | −26.843 | 1.00 | 66.72 | | C |
| ANISOU | 2086 | C | LEU | C | 293 | 8784 | 10611 | 5955 | −94 | 1391 | −245 | C |
| ATOM | 2087 | O | LEU | C | 293 | 55.014 | 10.613 | −27.261 | 1.00 | 66.53 | | O |
| ANISOU | 2087 | O | LEU | C | 293 | 8725 | 10647 | 5906 | −131 | 1412 | −349 | O |
| ATOM | 2088 | CB | LEU | C | 293 | 52.968 | 13.168 | −26.902 | 1.00 | 65.55 | | C |
| ANISOU | 2088 | CB | LEU | C | 293 | 8573 | 10742 | 5593 | 125 | 1297 | −527 | C |
| ATOM | 2089 | CG | LEU | C | 293 | 51.441 | 13.011 | −26.720 | 1.00 | 69.42 | | C |
| ANISOU | 2089 | CG | LEU | C | 293 | 8952 | 11486 | 5937 | 129 | 1249 | −854 | C |
| ATOM | 2090 | CD1 | LEU | C | 293 | 50.691 | 14.014 | −27.555 | 1.00 | 69.84 | | C |
| ANISOU | 2090 | CD1 | LEU | C | 293 | 8989 | 11694 | 5853 | 436 | 1191 | −923 | C |
| ATOM | 2091 | CD2 | LEU | C | 293 | 50.960 | 11.616 | −27.079 | 1.00 | 71.44 | | C |
| ANISOU | 2091 | CD2 | LEU | C | 293 | 9121 | 11906 | 6119 | 35 | 1283 | −1051 | C |
| ATOM | 2092 | N | ARG | C | 294 | 56.153 | 12.584 | −26.948 | 1.00 | 62.37 | | N |
| ANISOU | 2092 | N | ARG | C | 294 | 8295 | 9909 | 5495 | −38 | 1414 | 23 | N |
| ATOM | 2093 | CA | ARG | C | 294 | 57.433 | 12.242 | −27.597 | 1.00 | 62.38 | | C |
| ANISOU | 2093 | CA | ARG | C | 294 | 8300 | 9847 | 5554 | −2 | 1480 | 219 | C |
| ATOM | 2094 | C | ARG | C | 294 | 58.212 | 11.246 | −26.763 | 1.00 | 64.86 | | C |
| ANISOU | 2094 | C | ARG | C | 294 | 8622 | 10011 | 6010 | −210 | 1477 | 239 | C |
| ATOM | 2095 | O | ARG | C | 294 | 58.699 | 10.267 | −27.323 | 1.00 | 62.57 | | O |
| ANISOU | 2095 | O | ARG | C | 294 | 8303 | 9753 | 5720 | −179 | 1506 | 215 | O |
| ATOM | 2096 | CB | ARG | C | 294 | 58.311 | 13.477 | −27.861 | 1.00 | 63.21 | | C |
| ANISOU | 2096 | CB | ARG | C | 294 | 8458 | 9854 | 5705 | 76 | 1505 | 498 | C |
| ATOM | 2097 | CG | ARG | C | 294 | 57.847 | 14.290 | −29.045 | 1.00 | 77.18 | | C |
| ANISOU | 2097 | CG | ARG | C | 294 | 10240 | 11769 | 7314 | 319 | 1515 | 536 | C |
| ATOM | 2098 | CD | ARG | C | 294 | 58.801 | 14.201 | −30.216 | 1.00 | 87.36 | | C |
| ANISOU | 2098 | CD | ARG | C | 294 | 11505 | 13136 | 8550 | 429 | 1605 | 730 | C |
| ATOM | 2099 | NE | ARG | C | 294 | 59.217 | 15.531 | −30.667 | 1.00 | 88.67 | | N |
| ANISOU | 2099 | NE | ARG | C | 294 | 11755 | 13231 | 8703 | 498 | 1613 | 994 | N |
| ATOM | 2100 | CZ | ARG | C | 294 | 60.240 | 16.211 | −30.154 | 1.00 | 98.47 | | C |
| ANISOU | 2100 | CZ | ARG | C | 294 | 13045 | 14275 | 10093 | 351 | 1624 | 1224 | C |
| ATOM | 2101 | NH1 | ARG | C | 294 | 60.976 | 15.686 | −29.181 | 1.00 | 84.19 | | N |
| ANISOU | 2101 | NH1 | ARG | C | 294 | 11199 | 12339 | 8452 | 152 | 1627 | 1217 | N |
| ATOM | 2102 | NH2 | ARG | C | 294 | 60.536 | 17.418 | −30.612 | 1.00 | 85.62 | | N |
| ANISOU | 2102 | NH2 | ARG | C | 294 | 11512 | 12572 | 8448 | 396 | 1622 | 1464 | N |
| ATOM | 2103 | N | GLU | C | 295 | 58.324 | 11.476 | −25.424 | 1.00 | 63.12 | | N |
| ANISOU | 2103 | N | GLU | C | 295 | 8447 | 9633 | 5904 | −407 | 1431 | 270 | N |
| ATOM | 2104 | CA | GLU | C | 295 | 59.015 | 10.547 | −24.515 | 1.00 | 63.59 | | C |
| ANISOU | 2104 | CA | GLU | C | 295 | 8533 | 9541 | 6089 | −607 | 1407 | 295 | C |
| ATOM | 2105 | C | GLU | C | 295 | 58.569 | 9.100 | −24.764 | 1.00 | 70.55 | | C |
| ANISOU | 2105 | C | GLU | C | 295 | 9405 | 10479 | 6921 | −651 | 1393 | 111 | C |
| ATOM | 2106 | O | GLU | C | 295 | 59.421 | 8.212 | −24.847 | 1.00 | 71.65 | | O |
| ANISOU | 2106 | O | GLU | C | 295 | 9561 | 10527 | 7135 | −667 | 1388 | 159 | O |
| ATOM | 2107 | CB | GLU | C | 295 | 58.794 | 10.925 | −23.050 | 1.00 | 64.28 | | C |
| ANISOU | 2107 | CB | GLU | C | 295 | 8663 | 9515 | 6245 | −810 | 1349 | 287 | C |
| ATOM | 2108 | CG | GLU | C | 295 | 60.082 | 11.213 | −22.298 | 1.00 | 76.80 | | C |
| ANISOU | 2108 | CG | GLU | C | 295 | 10282 | 10908 | 7990 | −912 | 1334 | 490 | C |
| ATOM | 2109 | CD | GLU | C | 295 | 60.737 | 12.555 | −22.578 | 1.00 | 99.53 | | C |
| ANISOU | 2109 | CD | GLU | C | 295 | 13162 | 13728 | 10927 | −820 | 1357 | 685 | C |
| ATOM | 2110 | OE1 | GLU | C | 295 | 60.256 | 13.297 | −23.470 | 1.00 | 93.21 | | O |
| ANISOU | 2110 | OE1 | GLU | C | 295 | 12355 | 13022 | 10039 | −651 | 1384 | 693 | O |
| ATOM | 2111 | OE2 | GLU | C | 295 | 61.741 | 12.864 | −21.893 | 1.00 | 87.90 | | O |
| ANISOU | 2111 | OE2 | GLU | C | 295 | 11702 | 12109 | 9586 | −924 | 1340 | 833 | O |
| ATOM | 2112 | N | LYS | C | 296 | 57.238 | 8.894 | −24.959 | 1.00 | 66.65 | | N |
| ANISOU | 2112 | N | LYS | C | 296 | 8881 | 10143 | 6298 | −653 | 1380 | −109 | N |
| ATOM | 2113 | CA | LYS | C | 296 | 56.601 | 7.626 | −25.288 | 1.00 | 66.34 | | C |
| ANISOU | 2113 | CA | LYS | C | 296 | 8834 | 10176 | 6198 | −709 | 1360 | −311 | C |
| ATOM | 2114 | C | LYS | C | 296 | 57.019 | 7.157 | −26.717 | 1.00 | 70.71 | | C |
| ANISOU | 2114 | C | LYS | C | 296 | 9347 | 10819 | 6702 | −480 | 1397 | −328 | C |
| ATOM | 2115 | O | LYS | C | 296 | 57.797 | 6.204 | −26.831 | 1.00 | 70.45 | | O |
| ANISOU | 2115 | O | LYS | C | 296 | 9346 | 10678 | 6744 | −492 | 1379 | −310 | O |
| ATOM | 2116 | CB | LYS | C | 296 | 55.079 | 7.775 | −25.152 | 1.00 | 68.91 | | C |
| ANISOU | 2116 | CB | LYS | C | 296 | 9104 | 10692 | 6388 | −770 | 1344 | −540 | C |
| ATOM | 2117 | N | GLN | C | 297 | 56.555 | 7.865 | −27.786 | 1.00 | 67.37 | | N |
| ANISOU | 2117 | N | GLN | C | 297 | 8856 | 10596 | 6147 | −254 | 1439 | −357 | N |
| ATOM | 2118 | CA | GLN | C | 297 | 56.848 | 7.586 | −29.207 | 1.00 | 67.91 | | C |
| ANISOU | 2118 | CA | GLN | C | 297 | 8870 | 10809 | 6125 | −16 | 1482 | −375 | C |
| ATOM | 2119 | C | GLN | C | 297 | 58.293 | 7.208 | −29.454 | 1.00 | 70.85 | | C |
| ANISOU | 2119 | C | GLN | C | 297 | 9250 | 11074 | 6596 | 33 | 1511 | −211 | C |
| ATOM | 2120 | O | GLN | C | 297 | 58.554 | 6.167 | −30.041 | 1.00 | 71.75 | | O |
| ANISOU | 2120 | O | GLN | C | 297 | 9347 | 11214 | 6703 | 96 | 1498 | −322 | O |
| ATOM | 2121 | CB | GLN | C | 297 | 56.536 | 8.803 | −30.109 | 1.00 | 70.15 | | C |
| ANISOU | 2121 | CB | GLN | C | 297 | 9113 | 11269 | 6274 | 214 | 1525 | −299 | C |
| ATOM | 2122 | CG | GLN | C | 297 | 55.054 | 9.153 | −30.296 | 1.00 | 91.77 | | C |
| ANISOU | 2122 | CG | GLN | C | 297 | 11807 | 14201 | 8861 | 282 | 1490 | −510 | C |
| ATOM | 2123 | CD | GLN | C | 297 | 54.856 | 10.483 | −31.013 | 1.00 | 108.93 | | C |
| ANISOU | 2123 | CD | GLN | C | 297 | 13982 | 16488 | 10919 | 514 | 1504 | −393 | C |
| ATOM | 2124 | NE2 | GLN | C | 297 | 53.683 | 10.665 | −31.610 | 1.00 | 95.67 | | N |
| ANISOU | 2124 | NE2 | GLN | C | 297 | 12245 | 15035 | 9070 | 669 | 1473 | −586 | N |
| ATOM | 2125 | OE1 | GLN | C | 297 | 55.738 | 11.361 | −31.034 | 1.00 | 104.24 | | O |
| ANISOU | 2125 | OE1 | GLN | C | 297 | 13446 | 15776 | 10385 | 551 | 1533 | −131 | O |
| ATOM | 2126 | N | VAL | C | 298 | 59.225 | 8.073 | −29.017 | 1.00 | 65.98 | | N |
| ANISOU | 2126 | N | VAL | C | 298 | 8651 | 10348 | 6070 | 8 | 1542 | 34 | N |
| ATOM | 2127 | CA | VAL | C | 298 | 60.675 | 7.959 | −29.194 | 1.00 | 65.45 | | C |
| ANISOU | 2127 | CA | VAL | C | 298 | 8560 | 10216 | 6093 | 49 | 1579 | 213 | C |
| ATOM | 2128 | C | VAL | C | 298 | 61.206 | 6.616 | −28.704 | 1.00 | 69.70 | | C |
| ANISOU | 2128 | C | VAL | C | 298 | 9128 | 10613 | 6741 | −41 | 1515 | 121 | C |
| ATOM | 2129 | O | VAL | C | 298 | 61.987 | 5.982 | −29.425 | 1.00 | 70.32 | | O |
| ANISOU | 2129 | O | VAL | C | 298 | 9153 | 10751 | 6815 | 99 | 1530 | 107 | O |
| ATOM | 2130 | CB | VAL | C | 298 | 61.391 | 9.189 | −28.562 | 1.00 | 67.88 | | C |
| ANISOU | 2130 | CB | VAL | C | 298 | 8888 | 10408 | 6495 | −33 | 1605 | 468 | C |
| ATOM | 2131 | CG1 | VAL | C | 298 | 62.653 | 8.837 | −27.779 | 1.00 | 66.72 | | C |
| ANISOU | 2131 | CG1 | VAL | C | 298 | 8740 | 10099 | 6512 | −145 | 1589 | 593 | C |
| ATOM | 2132 | CG2 | VAL | C | 298 | 61.669 | 10.252 | −29.613 | 1.00 | 68.53 | | C |
| ANISOU | 2132 | CG2 | VAL | C | 298 | 8931 | 10635 | 6472 | 132 | 1686 | 635 | C |
| ATOM | 2133 | N | CYS | C | 299 | 60.734 | 6.164 | −27.522 | 1.00 | 65.04 | | N |
| ANISOU | 2133 | N | CYS | C | 299 | 8627 | 9852 | 6232 | −262 | 1435 | 48 | N |
| ATOM | 2134 | CA | CYS | C | 299 | 61.180 | 4.918 | −26.917 | 1.00 | 64.79 | | C |
| ANISOU | 2134 | CA | CYS | C | 299 | 8669 | 9640 | 6308 | −369 | 1348 | −16 | C |
| ATOM | 2135 | C | CYS | C | 299 | 60.620 | 3.695 | −27.591 | 1.00 | 66.48 | | C |
| ANISOU | 2135 | C | CYS | C | 299 | 8900 | 9898 | 6460 | −307 | 1301 | −250 | C |
| ATOM | 2136 | O | CYS | C | 299 | 61.402 | 2.799 | −27.906 | 1.00 | 67.36 | | O |
| ANISOU | 2136 | O | CYS | C | 299 | 9020 | 9949 | 6625 | −205 | 1258 | −287 | O |
| ATOM | 2137 | CB | CYS | C | 299 | 60.912 | 4.907 | −25.421 | 1.00 | 65.02 | | C |
| ANISOU | 2137 | CB | CYS | C | 299 | 8800 | 9482 | 6424 | −640 | 1281 | 17 | C |
| ATOM | 2138 | SG | CYS | C | 299 | 61.967 | 6.042 | −24.488 | 1.00 | 68.62 | | S |
| ANISOU | 2138 | SG | CYS | C | 299 | 9248 | 9823 | 7001 | −714 | 1299 | 281 | S |
| ATOM | 2139 | N | TYR | C | 300 | 59.288 | 3.647 | −27.841 | 1.00 | 59.97 | | N |
| ANISOU | 2139 | N | TYR | C | 300 | 8071 | 9191 | 5524 | −355 | 1301 | −426 | N |
| ATOM | 2140 | CA | TYR | C | 300 | 58.667 | 2.493 | −28.508 | 1.00 | 59.48 | | C |
| ANISOU | 2140 | CA | TYR | C | 300 | 8021 | 9176 | 5402 | −319 | 1249 | −672 | C |
| ATOM | 2141 | C | TYR | C | 300 | 59.101 | 2.375 | −29.966 | 1.00 | 62.99 | | C |
| ANISOU | 2141 | C | TYR | C | 300 | 8366 | 9807 | 5762 | −14 | 1294 | −729 | C |
| ATOM | 2142 | O | TYR | C | 300 | 59.045 | 1.292 | −30.543 | 1.00 | 63.42 | | O |
| ANISOU | 2142 | O | TYR | C | 300 | 8437 | 9852 | 5809 | 60 | 1233 | −915 | O |
| ATOM | 2143 | CB | TYR | C | 300 | 57.140 | 2.490 | −28.352 | 1.00 | 59.97 | | C |
| ANISOU | 2143 | CB | TYR | C | 300 | 8074 | 9355 | 5359 | −462 | 1241 | −857 | C |
| ATOM | 2144 | CG | TYR | C | 300 | 56.688 | 2.451 | −26.909 | 1.00 | 60.38 | | C |
| ANISOU | 2144 | CG | TYR | C | 300 | 8213 | 9257 | 5471 | −782 | 1197 | −826 | C |
| ATOM | 2145 | CD1 | TYR | C | 300 | 56.711 | 1.264 | −26.182 | 1.00 | 62.82 | | C |
| ANISOU | 2145 | CD1 | TYR | C | 300 | 8657 | 9347 | 5866 | −1008 | 1099 | −879 | C |
| ATOM | 2146 | CD2 | TYR | C | 300 | 56.248 | 3.601 | −26.264 | 1.00 | 59.65 | | C |
| ANISOU | 2146 | CD2 | TYR | C | 300 | 8079 | 9243 | 5343 | −857 | 1243 | −743 | C |
| ATOM | 2147 | CE1 | TYR | C | 300 | 56.309 | 1.225 | −24.845 | 1.00 | 61.52 | | C |
| ANISOU | 2147 | CE1 | TYR | C | 300 | 8575 | 9071 | 5730 | −1318 | 1065 | −831 | C |
| ATOM | 2148 | CE2 | TYR | C | 300 | 55.828 | 3.570 | −24.933 | 1.00 | 59.54 | | C |
| ANISOU | 2148 | CE2 | TYR | C | 300 | 8128 | 9134 | 5360 | −1147 | 1208 | −728 | C |
| ATOM | 2149 | CZ | TYR | C | 300 | 55.862 | 2.380 | −24.228 | 1.00 | 64.65 | | C |
| ANISOU | 2149 | CZ | TYR | C | 300 | 8900 | 9591 | 6073 | −1385 | 1127 | −764 | C |
| ATOM | 2150 | OH | TYR | C | 300 | 55.468 | 2.351 | −22.914 | 1.00 | 65.43 | | O |
| ANISOU | 2150 | OH | TYR | C | 300 | 9062 | 9621 | 6178 | −1683 | 1098 | −730 | O |
| ATOM | 2151 | N | HIS | C | 301 | 59.590 | 3.469 | −30.539 | 1.00 | 59.20 | | N |
| ANISOU | 2151 | N | HIS | C | 301 | 7790 | 9486 | 5217 | 157 | 1394 | −564 | N |
| ATOM | 2152 | CA | HIS | C | 301 | 60.078 | 3.411 | −31.887 | 1.00 | 61.05 | | C |
| ANISOU | 2152 | CA | HIS | C | 301 | 7920 | 9931 | 5345 | 433 | 1450 | −589 | C |
| ATOM | 2153 | C | HIS | C | 301 | 61.456 | 2.786 | −31.950 | 1.00 | 65.91 | | C |
| ANISOU | 2153 | C | HIS | C | 301 | 8521 | 10461 | 6062 | 519 | 1431 | −530 | C |
| ATOM | 2154 | O | HIS | C | 301 | 61.719 | 1.955 | −32.825 | 1.00 | 67.62 | | O |
| ANISOU | 2154 | O | HIS | C | 301 | 8688 | 10777 | 6227 | 705 | 1409 | −686 | O |
| ATOM | 2155 | CB | HIS | C | 301 | 60.038 | 4.767 | −32.577 | 1.00 | 62.53 | | C |
| ANISOU | 2155 | CB | HIS | C | 301 | 8024 | 10336 | 5400 | 574 | 1561 | −426 | C |
| ATOM | 2156 | CG | HIS | C | 301 | 60.169 | 4.618 | −34.059 | 1.00 | 67.91 | | C |
| ANISOU | 2156 | CG | HIS | C | 301 | 8599 | 11291 | 5912 | 846 | 1615 | −502 | C |
| ATOM | 2157 | CD2 | HIS | C | 301 | 59.415 | 3.894 | −34.919 | 1.00 | 70.84 | | C |
| ANISOU | 2157 | CD2 | HIS | C | 301 | 8933 | 11825 | 6159 | 974 | 1584 | −760 | C |
| ATOM | 2158 | ND1 | HIS | C | 301 | 61.236 | 5.162 | −34.744 | 1.00 | 70.54 | | N |
| ANISOU | 2158 | ND1 | HIS | C | 301 | 8846 | 11765 | 6192 | 997 | 1708 | −313 | N |
| ATOM | 2159 | CE1 | HIS | C | 301 | 61.072 | 4.792 | −35.997 | 1.00 | 71.75 | | C |
| ANISOU | 2159 | CE1 | HIS | C | 301 | 8913 | 12176 | 6174 | 1222 | 1735 | −453 | C |
| ATOM | 2160 | NE2 | HIS | C | 301 | 59.989 | 4.026 | −36.147 | 1.00 | 72.32 | | N |
| ANISOU | 2160 | NE2 | HIS | C | 301 | 9013 | 12264 | 6203 | 1225 | 1657 | −730 | N |
| ATOM | 2161 | N | VAL | C | 302 | 62.321 | 3.149 | −31.004 | 1.00 | 61.36 | | N |
| ANISOU | 2161 | N | VAL | C | 302 | 7979 | 9709 | 5624 | 396 | 1426 | −331 | N |
| ATOM | 2162 | CA | VAL | C | 302 | 63.661 | 2.579 | −30.901 | 1.00 | 62.18 | | C |
| ANISOU | 2162 | CA | VAL | C | 302 | 8059 | 9733 | 5834 | 473 | 1391 | −281 | C |
| ATOM | 2163 | C | VAL | C | 302 | 63.551 | 1.076 | −30.593 | 1.00 | 66.21 | | C |
| ANISOU | 2163 | C | VAL | C | 302 | 8685 | 10038 | 6433 | 443 | 1240 | −496 | C |
| ATOM | 2164 | O | VAL | C | 302 | 64.437 | 0.310 | −30.963 | 1.00 | 67.03 | | O |
| ANISOU | 2164 | O | VAL | C | 302 | 8759 | 10132 | 6578 | 609 | 1186 | −569 | O |
| ATOM | 2165 | CB | VAL | C | 302 | 64.499 | 3.352 | −29.860 | 1.00 | 65.58 | | C |
| ANISOU | 2165 | CB | VAL | C | 302 | 8497 | 10030 | 6391 | 329 | 1411 | −29 | C |
| ATOM | 2166 | CG1 | VAL | C | 302 | 65.765 | 2.595 | −29.469 | 1.00 | 66.37 | | C |
| ANISOU | 2166 | CG1 | VAL | C | 302 | 8590 | 10009 | 6620 | 377 | 1335 | −14 | C |
| ATOM | 2167 | CG2 | VAL | C | 302 | 64.844 | 4.740 | −30.375 | 1.00 | 65.32 | | C |
| ANISOU | 2167 | CG2 | VAL | C | 302 | 8350 | 10193 | 6274 | 389 | 1550 | 182 | C |
| ATOM | 2168 | N | LEU | C | 303 | 62.433 | 0.663 | −29.959 | 1.00 | 61.98 | | N |
| ANISOU | 2168 | N | LEU | C | 303 | 8282 | 9353 | 5916 | 234 | 1167 | −607 | N |
| ATOM | 2169 | CA | LEU | C | 303 | 62.150 | −0.730 | −29.632 | 1.00 | 62.53 | | C |
| ANISOU | 2169 | CA | LEU | C | 303 | 8498 | 9197 | 6064 | 150 | 1014 | −797 | C |
| ATOM | 2170 | C | LEU | C | 303 | 61.952 | −1.511 | −30.923 | 1.00 | 68.87 | | C |
| ANISOU | 2170 | C | LEU | C | 303 | 9247 | 10142 | 6777 | 380 | 988 | −1046 | C |
| ATOM | 2171 | O | LEU | C | 303 | 62.761 | −2.389 | −31.219 | 1.00 | 70.11 | | O |
| ANISOU | 2171 | O | LEU | C | 303 | 9426 | 10214 | 6999 | 540 | 897 | −1140 | O |
| ATOM | 2172 | CB | LEU | C | 303 | 60.939 | −0.852 | −28.687 | 1.00 | 61.47 | | C |
| ANISOU | 2172 | CB | LEU | C | 303 | 8492 | 8925 | 5939 | −167 | 969 | −837 | C |
| ATOM | 2173 | CG | LEU | C | 303 | 60.440 | −2.262 | −28.397 | 1.00 | 67.03 | | C |
| ANISOU | 2173 | CG | LEU | C | 303 | 9369 | 9395 | 6705 | −314 | 814 | −1024 | C |
| ATOM | 2174 | CD1 | LEU | C | 303 | 61.154 | −2.885 | −27.198 | 1.00 | 66.59 | | C |
| ANISOU | 2174 | CD1 | LEU | C | 303 | 9487 | 9004 | 6810 | −450 | 684 | −905 | C |
| ATOM | 2175 | CD2 | LEU | C | 303 | 58.955 | −2.253 | −28.189 | 1.00 | 70.83 | | C |
| ANISOU | 2175 | CD2 | LEU | C | 303 | 9882 | 9924 | 7105 | −568 | 825 | −1135 | C |
| ATOM | 2176 | N | GLY | C | 304 | 60.951 | −1.123 | −31.713 | 1.00 | 66.03 | | N |
| ANISOU | 2176 | N | GLY | C | 304 | 8805 | 10019 | 6264 | 427 | 1063 | −1155 | N |
| ATOM | 2177 | CA | GLY | C | 304 | 60.643 | −1.751 | −32.993 | 1.00 | 68.21 | | C |
| ANISOU | 2177 | CA | GLY | C | 304 | 9011 | 10480 | 6426 | 648 | 1047 | −1406 | C |
| ATOM | 2178 | C | GLY | C | 304 | 61.817 | −1.761 | −33.948 | 1.00 | 75.27 | | C |
| ANISOU | 2178 | C | GLY | C | 304 | 9770 | 11558 | 7270 | 969 | 1092 | −1395 | C |
| ATOM | 2179 | O | GLY | C | 304 | 61.924 | −2.644 | −34.812 | 1.00 | 76.80 | | O |
| ANISOU | 2179 | O | GLY | C | 304 | 9934 | 11830 | 7418 | 1171 | 1030 | −1630 | O |
| ATOM | 2180 | N | LEU | C | 305 | 62.713 | −0.770 | −33.770 | 1.00 | 72.06 | | N |
| ANISOU | 2180 | N | LEU | C | 305 | 9276 | 11233 | 6871 | 1006 | 1198 | −1131 | N |
| ATOM | 2181 | CA | LEU | C | 305 | 63.946 | −0.602 | −34.533 | 1.00 | 73.38 | | C |
| ANISOU | 2181 | CA | LEU | C | 305 | 9286 | 11610 | 6984 | 1263 | 1267 | −1070 | C |
| ATOM | 2182 | C | LEU | C | 305 | 64.955 | −1.684 | −34.120 | 1.00 | 77.14 | | C |
| ANISOU | 2182 | C | LEU | C | 305 | 9814 | 11878 | 7617 | 1333 | 1131 | −1151 | C |
| ATOM | 2183 | O | LEU | C | 305 | 65.617 | −2.257 | −34.988 | 1.00 | 78.02 | | O |
| ANISOU | 2183 | O | LEU | C | 305 | 9816 | 12155 | 7674 | 1601 | 1116 | −1293 | O |
| ATOM | 2184 | CB | LEU | C | 305 | 64.510 | 0.817 | −34.293 | 1.00 | 72.47 | | C |
| ANISOU | 2184 | CB | LEU | C | 305 | 9078 | 11617 | 6840 | 1209 | 1416 | −748 | C |
| ATOM | 2185 | CG | LEU | C | 305 | 64.400 | 1.860 | −35.428 | 1.00 | 77.62 | | C |
| ANISOU | 2185 | CG | LEU | C | 305 | 9581 | 12636 | 7274 | 1364 | 1573 | −655 | C |
| ATOM | 2186 | CD1 | LEU | C | 305 | 63.018 | 1.888 | −36.051 | 1.00 | 77.73 | | C |
| ANISOU | 2186 | CD1 | LEU | C | 305 | 9627 | 12761 | 7147 | 1384 | 1571 | −814 | C |
| ATOM | 2187 | CD2 | LEU | C | 305 | 64.737 | 3.247 | −34.916 | 1.00 | 79.03 | | C |
| ANISOU | 2187 | CD2 | LEU | C | 305 | 9736 | 12828 | 7466 | 1236 | 1685 | −326 | C |
| ATOM | 2188 | N | VAL | C | 306 | 65.032 | −1.988 | −32.799 | 1.00 | 72.33 | | N |
| ANISOU | 2188 | N | VAL | C | 306 | 9374 | 10918 | 7190 | 1105 | 1022 | −1074 | N |
| ATOM | 2189 | CA | VAL | C | 306 | 65.908 | −3.024 | −32.232 | 1.00 | 73.27 | | C |
| ANISOU | 2189 | CA | VAL | C | 306 | 9590 | 10780 | 7471 | 1153 | 858 | −1136 | C |
| ATOM | 2190 | C | VAL | C | 306 | 65.410 | −4.411 | −32.685 | 1.00 | 80.36 | | C |
| ANISOU | 2190 | C | VAL | C | 306 | 10607 | 11541 | 8385 | 1252 | 693 | −1455 | C |
| ATOM | 2191 | O | VAL | C | 306 | 66.187 | −5.189 | −33.252 | 1.00 | 81.83 | | O |
| ANISOU | 2191 | O | VAL | C | 306 | 10745 | 11761 | 8586 | 1522 | 607 | −1622 | O |
| ATOM | 2192 | CB | VAL | C | 306 | 66.083 | −2.901 | −30.685 | 1.00 | 74.91 | | C |
| ANISOU | 2192 | CB | VAL | C | 306 | 9958 | 10664 | 7842 | 872 | 786 | −941 | C |
| ATOM | 2193 | CG1 | VAL | C | 306 | 66.751 | −4.140 | −30.099 | 1.00 | 76.18 | | C |
| ANISOU | 2193 | CG1 | VAL | C | 306 | 10286 | 10500 | 8159 | 906 | 570 | −1042 | C |
| ATOM | 2194 | CG2 | VAL | C | 306 | 66.884 | −1.653 | −30.311 | 1.00 | 73.14 | | C |
| ANISOU | 2194 | CG2 | VAL | C | 306 | 9596 | 10566 | 7627 | 839 | 917 | −663 | C |
| ATOM | 2195 | N | HIS | C | 307 | 64.098 | −4.673 | −32.505 | 1.00 | 77.31 | | N |
| ANISOU | 2195 | N | HIS | C | 307 | 10355 | 11034 | 7985 | 1042 | 653 | −1556 | N |
| ATOM | 2196 | CA | HIS | C | 307 | 63.420 | −5.915 | −32.897 | 1.00 | 79.39 | | C |
| ANISOU | 2196 | CA | HIS | C | 307 | 10748 | 11150 | 8265 | 1060 | 499 | −1857 | C |
| ATOM | 2197 | C | HIS | C | 307 | 63.488 | −6.183 | −34.396 | 1.00 | 87.44 | | C |
| ANISOU | 2197 | C | HIS | C | 307 | 11604 | 12478 | 9141 | 1395 | 529 | −2100 | C |
| ATOM | 2198 | O | HIS | C | 307 | 63.037 | −7.233 | −34.856 | 1.00 | 88.50 | | O |
| ANISOU | 2198 | O | HIS | C | 307 | 11826 | 12513 | 9286 | 1456 | 393 | −2383 | O |
| ATOM | 2199 | CB | HIS | C | 307 | 61.966 | −5.887 | −32.431 | 1.00 | 78.99 | | C |
| ANISOU | 2199 | CB | HIS | C | 307 | 10813 | 11006 | 8193 | 731 | 499 | −1885 | C |
| ATOM | 2200 | CG | HIS | C | 307 | 61.806 | −6.096 | −30.964 | 1.00 | 81.43 | | C |
| ANISOU | 2200 | CG | HIS | C | 307 | 11331 | 10966 | 8643 | 392 | 413 | −1729 | C |
| ATOM | 2201 | CD2 | HIS | C | 307 | 62.507 | −5.572 | −29.930 | 1.00 | 81.86 | | C |
| ANISOU | 2201 | CD2 | HIS | C | 307 | 11415 | 10905 | 8781 | 284 | 435 | −1465 | C |
| ATOM | 2202 | ND1 | HIS | C | 307 | 60.823 | −6.921 | −30.466 | 1.00 | 84.00 | | N |
| ANISOU | 2202 | ND1 | HIS | C | 307 | 11855 | 11043 | 9018 | 116 | 289 | −1848 | N |
| ATOM | 2203 | CE1 | HIS | C | 307 | 60.949 | −6.871 | −29.150 | 1.00 | 82.76 | | C |
| ANISOU | 2203 | CE1 | HIS | C | 307 | 11846 | 10640 | 8959 | −149 | 246 | −1643 | C |
| ATOM | 2204 | NE2 | HIS | C | 307 | 61.948 | −6.068 | −28.783 | 1.00 | 81.69 | | N |
| ANISOU | 2204 | NE2 | HIS | C | 307 | 11614 | 10574 | 8848 | −48 | 326 | −1418 | N |
| ATOM | 2205 | N | ALA | C | 308 | 64.046 | −5.223 | −35.149 | 1.00 | 86.25 | | N |
| ANISOU | 2205 | N | ALA | C | 308 | 11219 | 12702 | 8848 | 1596 | 705 | −1986 | N |
| ATOM | 2206 | CA | ALA | C | 308 | 64.266 | −5.303 | −36.587 | 1.00 | 89.31 | | C |
| ANISOU | 2206 | CA | ALA | C | 308 | 11417 | 13458 | 9061 | 1928 | 765 | −2170 | C |
| ATOM | 2207 | C | ALA | C | 308 | 65.744 | −5.652 | −36.867 | 1.00 | 96.95 | | C |
| ANISOU | 2207 | C | ALA | C | 308 | 12273 | 14502 | 10061 | 2212 | 728 | −2200 | C |
| ATOM | 2208 | O | ALA | C | 308 | 66.045 | −6.397 | −37.812 | 1.00 | 99.89 | | O |
| ANISOU | 2208 | O | ALA | C | 308 | 12577 | 15003 | 10373 | 2500 | 653 | −2477 | O |
| ATOM | 2209 | CB | ALA | C | 308 | 63.908 | −3.977 | −37.237 | 1.00 | 89.06 | | C |
| ANISOU | 2209 | CB | ALA | C | 308 | 11205 | 13808 | 8825 | 1948 | 985 | −1997 | C |
| ATOM | 2210 | N | LEU | C | 309 | 66.656 | −5.108 | −36.031 | 1.00 | 91.99 | | N |
| ANISOU | 2210 | N | LEU | C | 309 | 11616 | 13810 | 9524 | 2135 | 776 | −1935 | N |
| ATOM | 2211 | CA | LEU | C | 309 | 68.097 | −5.321 | −36.119 | 1.00 | 92.74 | | C |
| ANISOU | 2211 | CA | LEU | C | 309 | 11582 | 13997 | 9657 | 2369 | 750 | −1933 | C |
| ATOM | 2212 | C | LEU | C | 309 | 68.376 | −6.742 | −35.640 | 1.00 | 98.05 | | C |
| ANISOU | 2212 | C | LEU | C | 309 | 12452 | 14290 | 10514 | 2448 | 483 | −2159 | C |
| ATOM | 2213 | O | LEU | C | 309 | 68.512 | −7.640 | −36.470 | 1.00 | 99.23 | | O |
| ANISOU | 2213 | O | LEU | C | 309 | 12568 | 14509 | 10625 | 2729 | 375 | −2463 | O |
| ATOM | 2214 | CB | LEU | C | 309 | 68.811 | −4.250 | −35.266 | 1.00 | 90.75 | | C |
| ANISOU | 2214 | CB | LEU | C | 309 | 11262 | 13758 | 9461 | 2200 | 869 | −1580 | C |
| ATOM | 2215 | CG | LEU | C | 309 | 70.331 | −4.181 | −35.302 | 1.00 | 96.33 | | C |
| ANISOU | 2215 | CG | LEU | C | 309 | 11782 | 14629 | 10188 | 2396 | 883 | −1530 | C |
| ATOM | 2216 | CD1 | LEU | C | 309 | 70.838 | −3.973 | −36.699 | 1.00 | 98.14 | | C |
| ANISOU | 2216 | CD1 | LEU | C | 309 | 11735 | 15353 | 10203 | 2695 | 1012 | −1641 | C |
| ATOM | 2217 | CD2 | LEU | C | 309 | 70.821 | −3.056 | −34.430 | 1.00 | 97.09 | | C |
| ANISOU | 2217 | CD2 | LEU | C | 309 | 11829 | 14719 | 10342 | 2171 | 1000 | −1184 | C |
| ATOM | 2218 | N | TRP | C | 310 | 68.386 | −6.955 | −34.315 | 1.00 | 94.72 | | N |
| ANISOU | 2218 | N | TRP | C | 310 | 12250 | 13458 | 10282 | 2200 | 364 | −2018 | N |
| ATOM | 2219 | CA | TRP | C | 310 | 68.547 | −8.271 | −33.714 | 1.00 | 96.84 | | C |
| ANISOU | 2219 | CA | TRP | C | 310 | 12767 | 13298 | 10732 | 2224 | 91 | −2184 | C |
| ATOM | 2220 | C | TRP | C | 310 | 67.154 | −8.642 | −33.326 | 1.00 | 101.15 | | C |
| ANISOU | 2220 | C | TRP | C | 310 | 13553 | 13564 | 11315 | 1918 | 24 | −2227 | C |
| ATOM | 2221 | O | TRP | C | 310 | 66.600 | −8.058 | −32.395 | 1.00 | 98.72 | | O |
| ANISOU | 2221 | O | TRP | C | 310 | 13332 | 13141 | 11036 | 1586 | 96 | −1997 | O |
| ATOM | 2222 | CB | TRP | C | 310 | 69.442 | −8.234 | −32.469 | 1.00 | 95.21 | | C |
| ANISOU | 2222 | CB | TRP | C | 310 | 12649 | 12842 | 10686 | 2133 | 4 | −1974 | C |
| ATOM | 2223 | CG | TRP | C | 310 | 70.734 | −7.520 | −32.675 | 1.00 | 96.52 | | C |
| ANISOU | 2223 | CG | TRP | C | 310 | 12533 | 13338 | 10800 | 2338 | 124 | −1860 | C |
| ATOM | 2224 | CD1 | TRP | C | 310 | 71.768 | −7.904 | −33.476 | 1.00 | 102.02 | | C |
| ANISOU | 2224 | CD1 | TRP | C | 310 | 13030 | 14289 | 11444 | 2720 | 93 | −2054 | C |
| ATOM | 2225 | CD2 | TRP | C | 310 | 71.129 | −6.293 | −32.068 | 1.00 | 94.25 | | C |
| ANISOU | 2225 | CD2 | TRP | C | 310 | 12120 | 13185 | 10505 | 2158 | 296 | −1540 | C |
| ATOM | 2226 | CE2 | TRP | C | 310 | 72.424 | −5.987 | −32.541 | 1.00 | 99.65 | | C |
| ANISOU | 2226 | CE2 | TRP | C | 310 | 12525 | 14206 | 11132 | 2420 | 368 | −1541 | C |
| ATOM | 2227 | CE3 | TRP | C | 310 | 70.512 | −5.413 | −31.166 | 1.00 | 92.87 | | C |
| ANISOU | 2227 | CE3 | TRP | C | 310 | 12034 | 12889 | 10363 | 1799 | 391 | −1266 | C |
| ATOM | 2228 | NE1 | TRP | C | 310 | 72.793 | −6.993 | −33.394 | 1.00 | 101.15 | | N |
| ANISOU | 2228 | NE1 | TRP | C | 310 | 12665 | 14479 | 11287 | 2767 | 245 | −1862 | N |
| ATOM | 2229 | CZ2 | TRP | C | 310 | 73.117 | −4.841 | −32.140 | 1.00 | 97.72 | | C |
| ANISOU | 2229 | CZ2 | TRP | C | 310 | 12101 | 14154 | 10873 | 2308 | 529 | −1266 | C |
| ATOM | 2230 | CZ3 | TRP | C | 310 | 71.198 | −4.277 | −30.771 | 1.00 | 93.13 | | C |
| ANISOU | 2230 | CZ3 | TRP | C | 310 | 11900 | 13099 | 10387 | 1716 | 540 | −1006 | C |
| ATOM | 2231 | CH2 | TRP | C | 310 | 72.484 | −4.000 | −31.257 | 1.00 | 95.16 | | C |
| ANISOU | 2231 | CH2 | TRP | C | 310 | 11893 | 13667 | 10598 | 1956 | 607 | −1001 | C |
| ATOM | 2232 | N | GLN | C | 311 | 66.548 | −9.548 | −34.081 | 1.00 | 100.63 | | N |
| ANISOU | 2232 | N | GLN | C | 311 | 13571 | 13432 | 11232 | 2025 | −99 | −2535 | N |
| ATOM | 2233 | CA | GLN | C | 311 | 65.173 | −9.920 | −33.807 | 1.00 | 100.49 | | C |
| ANISOU | 2233 | CA | GLN | C | 311 | 13754 | 13194 | 11234 | 1716 | −153 | −2601 | C |
| ATOM | 2234 | C | GLN | C | 311 | 65.057 | −11.008 | −32.720 | 1.00 | 105.83 | | C |
| ANISOU | 2234 | C | GLN | C | 311 | 14780 | 13317 | 12112 | 1505 | −410 | −2619 | C |
| ATOM | 2235 | O | GLN | C | 311 | 65.850 | −11.958 | −32.707 | 1.00 | 108.38 | | O |
| ANISOU | 2235 | O | GLN | C | 311 | 15220 | 13409 | 12548 | 1726 | −623 | −2765 | O |
| ATOM | 2236 | CB | GLN | C | 311 | 64.399 | −10.276 | −35.096 | 1.00 | 103.21 | | C |
| ANISOU | 2236 | CB | GLN | C | 311 | 14007 | 13770 | 11441 | 1866 | −135 | −2909 | C |
| ATOM | 2237 | CG | GLN | C | 311 | 64.771 | −11.570 | −35.803 | 1.00 | 115.06 | | C |
| ANISOU | 2237 | CG | GLN | C | 311 | 15582 | 15143 | 12993 | 2162 | −356 | −3272 | C |
| ATOM | 2238 | CD | GLN | C | 311 | 63.629 | −11.974 | −36.690 | 1.00 | 129.57 | | C |
| ANISOU | 2238 | CD | GLN | C | 311 | 17419 | 17079 | 14733 | 2137 | −370 | −3550 | C |
| ATOM | 2239 | NE2 | GLN | C | 311 | 62.663 | −12.682 | −36.118 | 1.00 | 120.87 | | N |
| ANISOU | 2239 | NE2 | GLN | C | 311 | 16575 | 15611 | 13739 | 1810 | −509 | −3614 | N |
| ATOM | 2240 | OE1 | GLN | C | 311 | 63.563 | −11.598 | −37.865 | 1.00 | 124.32 | | O |
| ANISOU | 2240 | OE1 | GLN | C | 311 | 16516 | 16841 | 13880 | 2384 | −247 | −3693 | O |
| ATOM | 2241 | N | PRO | C | 312 | 64.066 | −10.861 | −31.802 | 1.00 | 99.80 | | N |
| ANISOU | 2241 | N | PRO | C | 312 | 14187 | 12349 | 11382 | 1078 | −397 | −2466 | N |
| ATOM | 2242 | CA | PRO | C | 312 | 63.862 | −11.871 | −30.755 | 1.00 | 102.68 | | C |
| ANISOU | 2242 | CA | PRO | C | 312 | 14902 | 12200 | 11912 | 823 | −631 | −2451 | C |
| ATOM | 2243 | C | PRO | C | 312 | 63.282 | −13.198 | −31.275 | 1.00 | 152.69 | | C |
| ANISOU | 2243 | C | PRO | C | 312 | 21426 | 18293 | 18295 | 836 | −837 | −2775 | C |
| ATOM | 2244 | O | PRO | C | 312 | 63.658 | −13.737 | −32.323 | 1.00 | 116.75 | | O |
| ANISOU | 2244 | O | PRO | C | 312 | 16843 | 13757 | 13761 | 1197 | −957 | −3039 | O |
| ATOM | 2245 | CB | PRO | C | 312 | 62.860 | −11.190 | −29.814 | 1.00 | 101.48 | | C |
| ANISOU | 2245 | CB | PRO | C | 312 | 14804 | 12030 | 11723 | 365 | −506 | −2220 | C |
| ATOM | 2246 | CG | PRO | C | 312 | 62.808 | −9.771 | −30.226 | 1.00 | 102.40 | | C |
| ANISOU | 2246 | CG | PRO | C | 312 | 14614 | 12600 | 11691 | 429 | −234 | −2089 | C |
| ATOM | 2247 | CD | PRO | C | 312 | 63.074 | −9.779 | −31.672 | 1.00 | 98.71 | | C |
| ANISOU | 2247 | CD | PRO | C | 312 | 13931 | 12453 | 11121 | 811 | −176 | −2310 | C |
| ATOM | 2248 | N | ILE | C | 313 | 64.803 | −12.609 | −27.781 | 1.00 | 110.96 | | N |
| ANISOU | 2248 | N | ILE | C | 313 | 16496 | 12402 | 13260 | 414 | −955 | −1973 | N |
| ATOM | 2249 | CA | ILE | C | 313 | 64.068 | −13.782 | −27.333 | 1.00 | 113.15 | | C |
| ANISOU | 2249 | CA | ILE | C | 313 | 17129 | 12238 | 13624 | 136 | −1173 | −2047 | C |
| ATOM | 2250 | C | ILE | C | 313 | 62.569 | −13.442 | −27.201 | 1.00 | 114.85 | | C |
| ANISOU | 2250 | C | ILE | C | 313 | 17333 | 12568 | 13735 | −291 | −1022 | −2021 | C |
| ATOM | 2251 | O | ILE | C | 313 | 62.242 | −12.440 | −26.553 | 1.00 | 111.62 | | O |
| ANISOU | 2251 | O | ILE | C | 313 | 16807 | 12353 | 13248 | −513 | −834 | −1795 | O |
| ATOM | 2252 | CB | ILE | C | 313 | 64.692 | −14.347 | −26.023 | 1.00 | 117.87 | | C |
| ANISOU | 2252 | CB | ILE | C | 313 | 18036 | 12395 | 14356 | 10 | −1387 | −1837 | C |
| ATOM | 2253 | CG1 | ILE | C | 313 | 66.190 | −14.683 | −26.211 | 1.00 | 120.03 | | C |
| ANISOU | 2253 | CG1 | ILE | C | 313 | 18286 | 12594 | 14724 | 478 | −1547 | −1899 | C |
| ATOM | 2254 | CG2 | ILE | C | 313 | 63.936 | −15.575 | −25.514 | 1.00 | 122.02 | | C |
| ANISOU | 2254 | CG2 | ILE | C | 313 | 18965 | 12432 | 14965 | −315 | −1622 | −1881 | C |
| ATOM | 2255 | CD1 | ILE | C | 313 | 67.164 | −13.645 | −25.676 | 1.00 | 123.84 | | C |
| ANISOU | 2255 | CD1 | ILE | C | 313 | 18540 | 13331 | 15182 | 605 | −1404 | −1669 | C |
| ATOM | 2256 | N | PRO | C | 314 | 61.656 | −14.243 | −27.824 | 1.00 | 113.03 | | N |
| ANISOU | 2256 | N | PRO | C | 314 | 17203 | 12245 | 13498 | −394 | −1105 | −2272 | N |
| ATOM | 2257 | CA | PRO | C | 314 | 60.213 | −13.935 | −27.744 | 1.00 | 111.66 | | C |
| ANISOU | 2257 | CA | PRO | C | 314 | 16983 | 12229 | 13213 | −792 | −964 | −2281 | C |
| ATOM | 2258 | C | PRO | C | 314 | 59.611 | −14.051 | −26.343 | 1.00 | 114.66 | | C |
| ANISOU | 2258 | C | PRO | C | 314 | 17601 | 12350 | 13615 | −1302 | −1003 | −2047 | C |
| ATOM | 2259 | O | PRO | C | 314 | 59.653 | −15.121 | −25.731 | 1.00 | 117.12 | | O |
| ANISOU | 2259 | O | PRO | C | 314 | 18253 | 12206 | 14042 | −1472 | −1237 | −2024 | O |
| ATOM | 2260 | CB | PRO | C | 314 | 59.572 | −14.927 | −28.728 | 1.00 | 116.12 | | C |
| ANISOU | 2260 | CB | PRO | C | 314 | 17608 | 12727 | 13787 | −743 | −1084 | −2633 | C |
| ATOM | 2261 | CG | PRO | C | 314 | 60.700 | −15.454 | −29.554 | 1.00 | 122.50 | | C |
| ANISOU | 2261 | CG | PRO | C | 314 | 18412 | 13456 | 14677 | −243 | −1235 | −2822 | C |
| ATOM | 2262 | CD | PRO | C | 314 | 61.888 | −15.442 | −28.653 | 1.00 | 117.95 | | C |
| ANISOU | 2262 | CD | PRO | C | 314 | 17968 | 12638 | 14211 | −147 | −1337 | −2584 | C |
| ATOM | 2263 | N | GLY | C | 315 | 59.058 | −12.937 | −25.863 | 1.00 | 107.23 | | N |
| ANISOU | 2263 | N | GLY | C | 315 | 16482 | 11710 | 12552 | −1532 | −778 | −1878 | N |
| ATOM | 2264 | CA | GLY | C | 315 | 58.436 | −12.825 | −24.549 | 1.00 | 106.12 | | C |
| ANISOU | 2264 | CA | GLY | C | 315 | 16494 | 11444 | 12383 | −2012 | −764 | −1656 | C |
| ATOM | 2265 | C | GLY | C | 315 | 59.401 | −12.379 | −23.474 | 1.00 | 107.65 | | C |
| ANISOU | 2265 | C | GLY | C | 315 | 16753 | 11524 | 12624 | −1994 | −777 | −1355 | C |
| ATOM | 2266 | O | GLY | C | 315 | 59.273 | −12.790 | −22.316 | 1.00 | 108.26 | | O |
| ANISOU | 2266 | O | GLY | C | 315 | 17076 | 11333 | 12724 | −2331 | −875 | −1170 | O |
| ATOM | 2267 | N | ARG | C | 316 | 60.370 | −11.524 | −23.846 | 1.00 | 101.41 | | N |
| ANISOU | 2267 | N | ARG | C | 316 | 15737 | 10954 | 11838 | −1615 | −676 | −1303 | N |
| ATOM | 2268 | CA | ARG | C | 316 | 61.382 | −11.024 | −22.916 | 1.00 | 99.66 | | C |
| ANISOU | 2268 | CA | ARG | C | 316 | 15531 | 10670 | 11665 | −1553 | −682 | −1043 | C |
| ATOM | 2269 | C | ARG | C | 316 | 61.397 | −9.481 | −22.796 | 1.00 | 99.04 | | C |
| ANISOU | 2269 | C | ARG | C | 316 | 15147 | 10998 | 11486 | −1521 | −425 | −905 | C |
| ATOM | 2270 | O | ARG | C | 316 | 62.443 | −8.896 | −22.492 | 1.00 | 97.76 | | O |
| ANISOU | 2270 | O | ARG | C | 316 | 14889 | 10890 | 11365 | −1315 | −398 | −764 | O |
| ATOM | 2271 | CB | ARG | C | 316 | 62.772 | −11.589 | −23.282 | 1.00 | 101.39 | | C |
| ANISOU | 2271 | CB | ARG | C | 316 | 15810 | 10699 | 12014 | −1133 | −856 | −1093 | C |
| ATOM | 2272 | CG | ARG | C | 316 | 63.011 | −13.053 | −22.877 | 1.00 | 114.09 | | C |
| ANISOU | 2272 | CG | ARG | C | 316 | 17804 | 11792 | 13752 | −1185 | −1167 | −1129 | C |
| ATOM | 2273 | CD | ARG | C | 316 | 63.283 | −13.285 | −21.389 | 1.00 | 125.58 | | C |
| ANISOU | 2273 | CD | ARG | C | 316 | 19525 | 12941 | 15250 | −1449 | −1294 | −849 | C |
| ATOM | 2274 | NE | ARG | C | 316 | 64.352 | −12.436 | −20.848 | 1.00 | 132.44 | | N |
| ANISOU | 2274 | NE | ARG | C | 316 | 20242 | 13950 | 16128 | −1262 | −1229 | −657 | N |
| ATOM | 2275 | CZ | ARG | C | 316 | 64.169 | −11.487 | −19.931 | 1.00 | 142.25 | | C |
| ANISOU | 2275 | CZ | ARG | C | 316 | 21379 | 15381 | 17290 | −1494 | −1067 | −436 | C |
| ATOM | 2276 | NH1 | ARG | C | 316 | 65.195 | −10.765 | −19.500 | 1.00 | 123.80 | | N |
| ANISOU | 2276 | NH1 | ARG | C | 316 | 18906 | 13155 | 14976 | −1313 | −1027 | −287 | N |
| ATOM | 2277 | NH2 | ARG | C | 316 | 62.959 | −11.257 | −19.436 | 1.00 | 130.01 | | N |
| ANISOU | 2277 | NH2 | ARG | C | 316 | 19845 | 13922 | 15630 | −1906 | −947 | −382 | N |
| ATOM | 2278 | N | VAL | C | 317 | 60.220 | −8.836 | −22.963 | 1.00 | 92.75 | | N |
| ANISOU | 2278 | N | VAL | C | 317 | 14208 | 10475 | 10559 | −1740 | −253 | −949 | N |
| ATOM | 2279 | CA | VAL | C | 317 | 60.076 | −7.372 | −22.897 | 1.00 | 88.88 | | C |
| ANISOU | 2279 | CA | VAL | C | 317 | 13452 | 10347 | 9972 | −1712 | −30 | −841 | C |
| ATOM | 2280 | C | VAL | C | 317 | 59.498 | −6.876 | −21.561 | 1.00 | 89.32 | | C |
| ANISOU | 2280 | C | VAL | C | 317 | 13556 | 10419 | 9964 | −2096 | 26 | −664 | C |
| ATOM | 2281 | O | VAL | C | 317 | 58.440 | −7.344 | −21.136 | 1.00 | 90.36 | | O |
| ANISOU | 2281 | O | VAL | C | 317 | 13797 | 10504 | 10033 | −2440 | 3 | −716 | O |
| ATOM | 2282 | CB | VAL | C | 317 | 59.274 | −6.812 | −24.103 | 1.00 | 92.16 | | C |
| ANISOU | 2282 | CB | VAL | C | 317 | 13621 | 11123 | 10271 | −1566 | 127 | −1033 | C |
| ATOM | 2283 | CG1 | VAL | C | 317 | 59.137 | −5.287 | −24.036 | 1.00 | 89.05 | | C |
| ANISOU | 2283 | CG1 | VAL | C | 317 | 12985 | 11059 | 9791 | −1493 | 329 | −910 | C |
| ATOM | 2284 | CG2 | VAL | C | 317 | 59.901 | −7.243 | −25.429 | 1.00 | 93.49 | | C |
| ANISOU | 2284 | CG2 | VAL | C | 317 | 13735 | 11308 | 10478 | −1184 | 72 | −1220 | C |
| ATOM | 2285 | N | LYS | C | 318 | 60.173 | −5.886 | −20.942 | 1.00 | 81.59 | | N |
| ANISOU | 2285 | N | LYS | C | 318 | 12475 | 9535 | 8989 | −2042 | 107 | −470 | N |
| ATOM | 2286 | CA | LYS | C | 318 | 59.747 | −5.243 | −19.696 | 1.00 | 79.33 | | C |
| ANISOU | 2286 | CA | LYS | C | 318 | 12195 | 9313 | 8633 | −2350 | 171 | −311 | C |
| ATOM | 2287 | C | LYS | C | 318 | 59.540 | −3.754 | −19.978 | 1.00 | 77.82 | | C |
| ANISOU | 2287 | C | LYS | C | 318 | 11728 | 9474 | 8365 | −2227 | 367 | −297 | C |
| ATOM | 2288 | O | LYS | C | 318 | 60.491 | −3.032 | −20.304 | 1.00 | 75.34 | | O |
| ANISOU | 2288 | O | LYS | C | 318 | 11291 | 9229 | 8108 | −1952 | 415 | −219 | O |
| ATOM | 2289 | CB | LYS | C | 318 | 60.775 | −5.443 | −18.563 | 1.00 | 82.53 | | C |
| ANISOU | 2289 | CB | LYS | C | 318 | 12769 | 9466 | 9123 | −2407 | 49 | −90 | C |
| ATOM | 2290 | CG | LYS | C | 318 | 61.118 | −6.891 | −18.218 | 1.00 | 102.14 | | C |
| ANISOU | 2290 | CG | LYS | C | 318 | 15554 | 11552 | 11703 | −2448 | −182 | −77 | C |
| ATOM | 2291 | CD | LYS | C | 318 | 62.506 | −6.985 | −17.563 | 1.00 | 111.95 | | C |
| ANISOU | 2291 | CD | LYS | C | 318 | 16872 | 12608 | 13058 | −2259 | −301 | 92 | C |
| ATOM | 2292 | CE | LYS | C | 318 | 63.359 | −8.123 | −18.092 | 1.00 | 126.86 | | C |
| ANISOU | 2292 | CE | LYS | C | 318 | 18919 | 14208 | 15075 | −1992 | −504 | 9 | C |
| ATOM | 2293 | NZ | LYS | C | 318 | 63.776 | −7.930 | −19.515 | 1.00 | 135.96 | | N |
| ANISOU | 2293 | NZ | LYS | C | 318 | 19857 | 15545 | 16255 | −1616 | −432 | −185 | N |
| ATOM | 2294 | N | ASP | C | 319 | 58.282 | −3.312 | −19.885 | 1.00 | 72.77 | | N |
| ANISOU | 2294 | N | ASP | C | 319 | 10993 | 9064 | 7594 | −2431 | 473 | −382 | N |
| ATOM | 2295 | CA | ASP | C | 319 | 57.880 | −1.933 | −20.137 | 1.00 | 70.13 | | C |
| ANISOU | 2295 | CA | ASP | C | 319 | 10424 | 9049 | 7174 | −2328 | 635 | −395 | C |
| ATOM | 2296 | C | ASP | C | 319 | 57.587 | −1.213 | −18.822 | 1.00 | 73.48 | | C |
| ANISOU | 2296 | C | ASP | C | 319 | 10838 | 9539 | 7541 | −2571 | 675 | −271 | C |
| ATOM | 2297 | O | ASP | C | 319 | 56.452 | −1.251 | −18.327 | 1.00 | 73.48 | | O |
| ANISOU | 2297 | O | ASP | C | 319 | 10825 | 9673 | 7421 | −2844 | 707 | −354 | O |
| ATOM | 2298 | CB | ASP | C | 319 | 56.675 | −1.905 | −21.103 | 1.00 | 71.95 | | C |
| ANISOU | 2298 | CB | ASP | C | 319 | 10524 | 9527 | 7285 | −2310 | 711 | −627 | C |
| ATOM | 2299 | CG | ASP | C | 319 | 56.020 | −0.559 | −21.387 | 1.00 | 76.74 | | C |
| ANISOU | 2299 | CG | ASP | C | 319 | 10911 | 10467 | 7779 | −2215 | 854 | −674 | C |
| ATOM | 2300 | OD1 | ASP | C | 319 | 56.615 | 0.486 | −21.034 | 1.00 | 74.65 | | O |
| ANISOU | 2300 | OD1 | ASP | C | 319 | 10580 | 10239 | 7543 | −2109 | 906 | −523 | O |
| ATOM | 2301 | OD2 | ASP | C | 319 | 54.918 | −0.552 | −21.981 | 1.00 | 83.17 | | O |
| ANISOU | 2301 | OD2 | ASP | C | 319 | 11624 | 11499 | 8478 | −2238 | 901 | −871 | O |
| ATOM | 2302 | N | TYR | C | 320 | 58.635 | −0.564 | −18.257 | 1.00 | 69.32 | | N |
| ANISOU | 2302 | N | TYR | C | 320 | 10303 | 8940 | 7095 | −2468 | 671 | −86 | N |
| ATOM | 2303 | CA | TYR | C | 320 | 58.563 | 0.219 | −17.014 | 1.00 | 68.14 | | C |
| ANISOU | 2303 | CA | TYR | C | 320 | 10136 | 8848 | 6906 | −2647 | 698 | 33 | C |
| ATOM | 2304 | C | TYR | C | 320 | 58.125 | 1.646 | −17.295 | 1.00 | 69.56 | | C |
| ANISOU | 2304 | C | TYR | C | 320 | 10107 | 9304 | 7019 | −2529 | 829 | −14 | C |
| ATOM | 2305 | O | TYR | C | 320 | 57.922 | 2.410 | −16.357 | 1.00 | 67.86 | | O |
| ANISOU | 2305 | O | TYR | C | 320 | 9853 | 9173 | 6759 | −2655 | 855 | 38 | O |
| ATOM | 2306 | CB | TYR | C | 320 | 59.884 | 0.179 | −16.221 | 1.00 | 69.32 | | C |
| ANISOU | 2306 | CB | TYR | C | 320 | 10384 | 8779 | 7176 | −2608 | 609 | 238 | C |
| ATOM | 2307 | CG | TYR | C | 320 | 60.218 | −1.208 | −15.731 | 1.00 | 73.67 | | C |
| ANISOU | 2307 | CG | TYR | C | 320 | 11176 | 9039 | 7776 | −2742 | 451 | 292 | C |
| ATOM | 2308 | CD1 | TYR | C | 320 | 59.582 | −1.746 | −14.616 | 1.00 | 77.32 | | C |
| ANISOU | 2308 | CD1 | TYR | C | 320 | 11794 | 9430 | 8152 | −3098 | 390 | 338 | C |
| ATOM | 2309 | CD2 | TYR | C | 320 | 61.109 | −2.017 | −16.427 | 1.00 | 75.53 | | C |
| ANISOU | 2309 | CD2 | TYR | C | 320 | 11489 | 9078 | 8131 | −2514 | 354 | 286 | C |
| ATOM | 2310 | CE1 | TYR | C | 320 | 59.832 | −3.054 | −14.200 | 1.00 | 81.02 | | C |
| ANISOU | 2310 | CE1 | TYR | C | 320 | 12522 | 9600 | 8661 | −3235 | 227 | 401 | C |
| ATOM | 2311 | CE2 | TYR | C | 320 | 61.384 | −3.317 | −16.009 | 1.00 | 78.72 | | C |
| ANISOU | 2311 | CE2 | TYR | C | 320 | 12142 | 9182 | 8587 | −2614 | 180 | 322 | C |
| ATOM | 2312 | CZ | TYR | C | 320 | 60.737 | −3.837 | −14.898 | 1.00 | 88.40 | | C |
| ANISOU | 2312 | CZ | TYR | C | 320 | 13553 | 10302 | 9734 | −2981 | 112 | 391 | C |
| ATOM | 2313 | OH | TYR | C | 320 | 61.003 | −5.123 | −14.484 | 1.00 | 91.08 | | O |
| ANISOU | 2313 | OH | TYR | C | 320 | 14174 | 10310 | 10123 | −3091 | −77 | 449 | O |
| ATOM | 2314 | N | ILE | C | 321 | 57.965 | 2.002 | −18.594 | 1.00 | 65.11 | | N |
| ANISOU | 2314 | N | ILE | C | 321 | 9418 | 8882 | 6441 | −2280 | 900 | −121 | N |
| ATOM | 2315 | CA | ILE | C | 321 | 57.497 | 3.321 | −19.025 | 1.00 | 63.19 | | C |
| ANISOU | 2315 | CA | ILE | C | 321 | 9002 | 8880 | 6129 | −2135 | 1004 | −170 | C |
| ATOM | 2316 | C | ILE | C | 321 | 55.981 | 3.382 | −18.749 | 1.00 | 68.26 | | C |
| ANISOU | 2316 | C | ILE | C | 321 | 9581 | 9745 | 6611 | −2329 | 1039 | −356 | C |
| ATOM | 2317 | O | ILE | C | 321 | 55.501 | 4.270 | −18.027 | 1.00 | 65.97 | | O |
| ANISOU | 2317 | O | ILE | C | 321 | 9216 | 9597 | 6253 | −2405 | 1073 | −371 | O |
| ATOM | 2318 | CB | ILE | C | 321 | 57.876 | 3.618 | −20.514 | 1.00 | 65.04 | | C |
| ANISOU | 2318 | CB | ILE | C | 321 | 9141 | 9191 | 6381 | −1803 | 1059 | −196 | C |
| ATOM | 2319 | CG1 | ILE | C | 321 | 59.383 | 3.411 | −20.749 | 1.00 | 64.76 | | C |
| ANISOU | 2319 | CG1 | ILE | C | 321 | 9148 | 8971 | 6486 | −1640 | 1025 | −37 | C |
| ATOM | 2320 | CG2 | ILE | C | 321 | 57.438 | 5.038 | −20.924 | 1.00 | 64.18 | | C |
| ANISOU | 2320 | CG2 | ILE | C | 321 | 8892 | 9290 | 6205 | −1648 | 1145 | −206 | C |
| ATOM | 2321 | CD1 | ILE | C | 321 | 59.811 | 3.420 | −22.150 | 1.00 | 69.33 | | C |
| ANISOU | 2321 | CD1 | ILE | C | 321 | 9645 | 9634 | 7063 | −1354 | 1070 | −70 | C |
| ATOM | 2322 | N | ALA | C | 322 | 55.246 | 2.397 | −19.291 | 1.00 | 68.25 | | N |
| ANISOU | 2322 | N | ALA | C | 322 | 9604 | 9779 | 6550 | −2413 | 1022 | −514 | N |
| ATOM | 2323 | CA | ALA | C | 322 | 53.811 | 2.317 | −19.098 | 1.00 | 70.02 | | C |
| ANISOU | 2323 | CA | ALA | C | 322 | 9747 | 10240 | 6616 | −2617 | 1054 | −712 | C |
| ATOM | 2324 | C | ALA | C | 322 | 53.558 | 2.039 | −17.641 | 1.00 | 75.49 | | C |
| ANISOU | 2324 | C | ALA | C | 322 | 10522 | 10896 | 7265 | −2974 | 1023 | −651 | C |
| ATOM | 2325 | O | ALA | C | 322 | 52.849 | 2.818 | −17.022 | 1.00 | 75.99 | | O |
| ANISOU | 2325 | O | ALA | C | 322 | 10470 | 11187 | 7216 | −3068 | 1072 | −719 | O |
| ATOM | 2326 | CB | ALA | C | 322 | 53.205 | 1.239 | −19.976 | 1.00 | 72.39 | | C |
| ANISOU | 2326 | CB | ALA | C | 322 | 10065 | 10561 | 6881 | −2650 | 1031 | −888 | C |
| ATOM | 2327 | N | VAL | C | 323 | 54.206 | 1.005 | −17.064 | 1.00 | 72.59 | | N |
| ANISOU | 2327 | N | VAL | C | 323 | 10355 | 10245 | 6981 | −3148 | 932 | −513 | N |
| ATOM | 2328 | CA | VAL | C | 323 | 54.072 | 0.691 | −15.633 | 1.00 | 72.67 | | C |
| ANISOU | 2328 | CA | VAL | C | 323 | 10474 | 10202 | 6937 | −3497 | 892 | −413 | C |
| ATOM | 2329 | C | VAL | C | 323 | 55.424 | 0.831 | −14.908 | 1.00 | 73.97 | | C |
| ANISOU | 2329 | C | VAL | C | 323 | 10762 | 10112 | 7231 | −3430 | 823 | −172 | C |
| ATOM | 2330 | O | VAL | C | 323 | 56.156 | −0.160 | −14.761 | 1.00 | 73.96 | | O |
| ANISOU | 2330 | O | VAL | C | 323 | 10955 | 9820 | 7325 | −3474 | 715 | −55 | O |
| ATOM | 2331 | CB | VAL | C | 323 | 53.372 | −0.653 | −15.321 | 1.00 | 78.89 | | C |
| ANISOU | 2331 | CB | VAL | C | 323 | 11405 | 10913 | 7656 | −3858 | 833 | −465 | C |
| ATOM | 2332 | CG1 | VAL | C | 323 | 53.058 | −0.764 | −13.837 | 1.00 | 79.73 | | C |
| ANISOU | 2332 | CG1 | VAL | C | 323 | 11594 | 11044 | 7657 | −4234 | 815 | −358 | C |
| ATOM | 2333 | CG2 | VAL | C | 323 | 52.102 | −0.809 | −16.142 | 1.00 | 79.87 | | C |
| ANISOU | 2333 | CG2 | VAL | C | 323 | 11381 | 11307 | 7660 | −3916 | 899 | −723 | C |
| ATOM | 2334 | N | PRO | C | 324 | 55.733 | 2.058 | −14.409 | 1.00 | 67.93 | | N |
| ANISOU | 2334 | N | PRO | C | 324 | 9888 | 9454 | 6468 | −3326 | 869 | −109 | N |
| ATOM | 2335 | CA | PRO | C | 324 | 56.992 | 2.265 | −13.666 | 1.00 | 66.59 | | C |
| ANISOU | 2335 | CA | PRO | C | 324 | 9811 | 9076 | 6413 | −3277 | 803 | 103 | C |
| ATOM | 2336 | C | PRO | C | 324 | 57.202 | 1.324 | −12.486 | 1.00 | 70.87 | | C |
| ANISOU | 2336 | C | PRO | C | 324 | 10553 | 9445 | 6929 | −3578 | 701 | 233 | C |
| ATOM | 2337 | O | PRO | C | 324 | 56.232 | 0.770 | −11.980 | 1.00 | 72.28 | | O |
| ANISOU | 2337 | O | PRO | C | 324 | 10774 | 9723 | 6966 | −3884 | 706 | 169 | O |
| ATOM | 2338 | CB | PRO | C | 324 | 56.875 | 3.715 | −13.187 | 1.00 | 66.98 | | C |
| ANISOU | 2338 | CB | PRO | C | 324 | 9704 | 9319 | 6425 | −3204 | 869 | 92 | C |
| ATOM | 2339 | CG | PRO | C | 324 | 55.946 | 4.363 | −14.144 | 1.00 | 70.77 | | C |
| ANISOU | 2339 | CG | PRO | C | 324 | 10014 | 10041 | 6833 | −3054 | 961 | −98 | C |
| ATOM | 2340 | CD | PRO | C | 324 | 54.957 | 3.313 | −14.503 | 1.00 | 67.97 | | C |
| ANISOU | 2340 | CD | PRO | C | 324 | 9687 | 9766 | 6375 | −3237 | 966 | −242 | C |
| ATOM | 2341 | N | LYS | C | 325 | 58.462 | 1.142 | −12.039 | 1.00 | 66.19 | | N |
| ANISOU | 2341 | N | LYS | C | 325 | 10081 | 8607 | 6459 | −3501 | 605 | 418 | N |
| ATOM | 2342 | CA | LYS | C | 325 | 58.769 | 0.315 | −10.861 | 1.00 | 66.74 | | C |
| ANISOU | 2342 | CA | LYS | C | 325 | 10361 | 8498 | 6498 | −3758 | 487 | 569 | C |
| ATOM | 2343 | C | LYS | C | 325 | 58.072 | 0.966 | −9.630 | 1.00 | 71.52 | | C |
| ANISOU | 2343 | C | LYS | C | 325 | 10898 | 9341 | 6936 | −4012 | 540 | 568 | C |
| ATOM | 2344 | O | LYS | C | 325 | 57.572 | 2.086 | −9.777 | 1.00 | 69.00 | | O |
| ANISOU | 2344 | O | LYS | C | 325 | 10372 | 9275 | 6570 | −3919 | 646 | 448 | O |
| ATOM | 2345 | CB | LYS | C | 325 | 60.292 | 0.203 | −10.665 | 1.00 | 67.97 | | C |
| ANISOU | 2345 | CB | LYS | C | 325 | 10613 | 8396 | 6816 | −3562 | 373 | 743 | C |
| ATOM | 2346 | CG | LYS | C | 325 | 60.901 | −1.076 | −11.225 | 1.00 | 76.58 | | C |
| ANISOU | 2346 | CG | LYS | C | 325 | 11895 | 9187 | 8015 | −3473 | 242 | 780 | C |
| ATOM | 2347 | CD | LYS | C | 325 | 62.408 | −1.151 | −10.969 | 1.00 | 85.90 | | C |
| ANISOU | 2347 | CD | LYS | C | 325 | 13130 | 10163 | 9344 | −3246 | 128 | 923 | C |
| ATOM | 2348 | CE | LYS | C | 325 | 63.065 | −2.352 | −11.618 | 1.00 | 100.66 | | C |
| ANISOU | 2348 | CE | LYS | C | 325 | 15169 | 11751 | 11328 | −3090 | −15 | 924 | C |
| ATOM | 2349 | NZ | LYS | C | 325 | 64.543 | −2.185 | −11.760 | 1.00 | 108.81 | | N |
| ANISOU | 2349 | NZ | LYS | C | 325 | 16148 | 12685 | 12509 | −2772 | −86 | 994 | N |
| ATOM | 2350 | N | PRO | C | 326 | 57.971 | 0.319 | −8.430 | 1.00 | 71.46 | | N |
| ANISOU | 2350 | N | PRO | C | 326 | 11053 | 9278 | 6821 | −4326 | 465 | 686 | N |
| ATOM | 2351 | CA | PRO | C | 326 | 57.279 | 0.966 | −7.297 | 1.00 | 70.98 | | C |
| ANISOU | 2351 | CA | PRO | C | 326 | 10895 | 9500 | 6573 | −4555 | 526 | 658 | C |
| ATOM | 2352 | C | PRO | C | 326 | 57.867 | 2.325 | −6.932 | 1.00 | 73.29 | | C |
| ANISOU | 2352 | C | PRO | C | 326 | 11031 | 9895 | 6923 | −4341 | 554 | 662 | C |
| ATOM | 2353 | O | PRO | C | 326 | 57.122 | 3.219 | −6.543 | 1.00 | 72.20 | | O |
| ANISOU | 2353 | O | PRO | C | 326 | 10719 | 10050 | 6664 | −4384 | 640 | 530 | O |
| ATOM | 2354 | CB | PRO | C | 326 | 57.469 | −0.045 | −6.176 | 1.00 | 74.80 | | C |
| ANISOU | 2354 | CB | PRO | C | 326 | 11623 | 9833 | 6963 | −4872 | 412 | 843 | C |
| ATOM | 2355 | CG | PRO | C | 326 | 58.740 | −0.795 | −6.560 | 1.00 | 79.73 | | C |
| ANISOU | 2355 | CG | PRO | C | 326 | 12453 | 10054 | 7786 | −4677 | 263 | 999 | C |
| ATOM | 2356 | CD | PRO | C | 326 | 58.508 | −0.994 | −8.013 | 1.00 | 75.19 | | C |
| ANISOU | 2356 | CD | PRO | C | 326 | 11813 | 9435 | 7322 | −4476 | 310 | 854 | C |
| ATOM | 2357 | N | ASN | C | 327 | 59.202 | 2.476 | −7.116 | 1.00 | 70.01 | | N |
| ANISOU | 2357 | N | ASN | C | 327 | 10668 | 9238 | 6695 | −4097 | 475 | 794 | N |
| ATOM | 2358 | CA | ASN | C | 327 | 60.001 | 3.686 | −6.888 | 1.00 | 68.48 | | C |
| ANISOU | 2358 | CA | ASN | C | 327 | 10350 | 9070 | 6601 | −3885 | 480 | 824 | C |
| ATOM | 2359 | C | ASN | C | 327 | 59.820 | 4.783 | −7.974 | 1.00 | 69.30 | | C |
| ANISOU | 2359 | C | ASN | C | 327 | 10253 | 9285 | 6792 | −3610 | 585 | 692 | C |
| ATOM | 2360 | O | ASN | C | 327 | 60.016 | 5.960 | −7.683 | 1.00 | 67.19 | | O |
| ANISOU | 2360 | O | ASN | C | 327 | 9869 | 9091 | 6569 | −3496 | 605 | 679 | O |
| ATOM | 2361 | CB | ASN | C | 327 | 61.491 | 3.323 | −6.702 | 1.00 | 69.10 | | C |
| ANISOU | 2361 | CB | ASN | C | 327 | 10552 | 8867 | 6836 | −3754 | 351 | 1011 | C |
| ATOM | 2362 | CG | ASN | C | 327 | 62.188 | 2.644 | −7.874 | 1.00 | 76.22 | | C |
| ANISOU | 2362 | CG | ASN | C | 327 | 11509 | 9549 | 7903 | −3526 | 313 | 1040 | C |
| ATOM | 2363 | ND2 | ASN | C | 327 | 63.250 | 1.904 | −7.579 | 1.00 | 62.88 | | N |
| ANISOU | 2363 | ND2 | ASN | C | 327 | 9962 | 7623 | 6306 | −3463 | 174 | 1183 | N |
| ATOM | 2364 | OD1 | ASN | C | 327 | 61.839 | 2.821 | −9.049 | 1.00 | 65.14 | | O |
| ANISOU | 2364 | OD1 | ASN | C | 327 | 10011 | 8200 | 6540 | −3380 | 399 | 927 | O |
| ATOM | 2365 | N | GLY | C | 328 | 59.465 | 4.387 | −9.195 | 1.00 | 65.48 | | N |
| ANISOU | 2365 | N | GLY | C | 328 | 9746 | 8800 | 6333 | −3508 | 639 | 603 | N |
| ATOM | 2366 | CA | GLY | C | 328 | 59.250 | 5.326 | −10.293 | 1.00 | 64.64 | | C |
| ANISOU | 2366 | CA | GLY | C | 328 | 9474 | 8802 | 6284 | −3253 | 731 | 493 | C |
| ATOM | 2367 | C | GLY | C | 328 | 60.132 | 5.132 | −11.514 | 1.00 | 69.39 | | C |
| ANISOU | 2367 | C | GLY | C | 328 | 10078 | 9242 | 7044 | −2987 | 731 | 548 | C |
| ATOM | 2368 | O | GLY | C | 328 | 60.087 | 5.940 | −12.455 | 1.00 | 68.02 | | O |
| ANISOU | 2368 | O | GLY | C | 328 | 9782 | 9153 | 6911 | −2773 | 806 | 488 | O |
| ATOM | 2369 | N | TYR | C | 329 | 60.928 | 4.042 | −11.511 | 1.00 | 67.20 | | N |
| ANISOU | 2369 | N | TYR | C | 329 | 9947 | 8741 | 6845 | −2993 | 639 | 658 | N |
| ATOM | 2370 | CA | TYR | C | 329 | 61.833 | 3.694 | −12.597 | 1.00 | 66.97 | | C |
| ANISOU | 2370 | CA | TYR | C | 329 | 9918 | 8574 | 6952 | −2741 | 623 | 695 | C |
| ATOM | 2371 | C | TYR | C | 329 | 61.067 | 3.447 | −13.864 | 1.00 | 70.96 | | C |
| ANISOU | 2371 | C | TYR | C | 329 | 10367 | 9178 | 7417 | −2639 | 699 | 547 | C |
| ATOM | 2372 | O | TYR | C | 329 | 60.147 | 2.630 | −13.886 | 1.00 | 71.82 | | O |
| ANISOU | 2372 | O | TYR | C | 329 | 10544 | 9316 | 7427 | −2807 | 692 | 447 | O |
| ATOM | 2373 | CB | TYR | C | 329 | 62.676 | 2.454 | −12.245 | 1.00 | 69.89 | | C |
| ANISOU | 2373 | CB | TYR | C | 329 | 10474 | 8691 | 7389 | −2775 | 482 | 800 | C |
| ATOM | 2374 | CG | TYR | C | 329 | 63.553 | 1.924 | −13.367 | 1.00 | 72.82 | | C |
| ANISOU | 2374 | CG | TYR | C | 329 | 10841 | 8941 | 7885 | −2503 | 450 | 801 | C |
| ATOM | 2375 | CD1 | TYR | C | 329 | 64.689 | 2.616 | −13.776 | 1.00 | 74.78 | | C |
| ANISOU | 2375 | CD1 | TYR | C | 329 | 10964 | 9198 | 8250 | −2272 | 469 | 874 | C |
| ATOM | 2376 | CD2 | TYR | C | 329 | 63.292 | 0.692 | −13.964 | 1.00 | 74.54 | | C |
| ANISOU | 2376 | CD2 | TYR | C | 329 | 11179 | 9041 | 8102 | −2490 | 392 | 722 | C |
| ATOM | 2377 | CE1 | TYR | C | 329 | 65.523 | 2.114 | −14.773 | 1.00 | 76.66 | | C |
| ANISOU | 2377 | CE1 | TYR | C | 329 | 11174 | 9372 | 8582 | −2022 | 444 | 863 | C |
| ATOM | 2378 | CE2 | TYR | C | 329 | 64.132 | 0.171 | −14.950 | 1.00 | 75.71 | | C |
| ANISOU | 2378 | CE2 | TYR | C | 329 | 11316 | 9094 | 8355 | −2220 | 349 | 699 | C |
| ATOM | 2379 | CZ | TYR | C | 329 | 65.249 | 0.886 | −15.349 | 1.00 | 83.07 | | C |
| ANISOU | 2379 | CZ | TYR | C | 329 | 12101 | 10076 | 9384 | −1981 | 381 | 767 | C |
| ATOM | 2380 | OH | TYR | C | 329 | 66.094 | 0.411 | −16.324 | 1.00 | 84.97 | | O |
| ANISOU | 2380 | OH | TYR | C | 329 | 12297 | 10280 | 9708 | −1709 | 350 | 730 | O |
| ATOM | 2381 | N | GLN | C | 330 | 61.457 | 4.162 | −14.915 | 1.00 | 66.73 | | N |
| ANISOU | 2381 | N | GLN | C | 330 | 9704 | 8702 | 6949 | −2375 | 768 | 539 | N |
| ATOM | 2382 | CA | GLN | C | 330 | 60.916 | 4.028 | −16.257 | 1.00 | 66.32 | | C |
| ANISOU | 2382 | CA | GLN | C | 330 | 9583 | 8756 | 6860 | −2218 | 838 | 411 | C |
| ATOM | 2383 | C | GLN | C | 330 | 62.070 | 3.706 | −17.177 | 1.00 | 73.39 | | C |
| ANISOU | 2383 | C | GLN | C | 330 | 10461 | 9555 | 7869 | −1968 | 823 | 472 | C |
| ATOM | 2384 | O | GLN | C | 330 | 63.150 | 4.311 | −17.069 | 1.00 | 73.67 | | O |
| ANISOU | 2384 | O | GLN | C | 330 | 10438 | 9554 | 7999 | −1857 | 825 | 602 | O |
| ATOM | 2385 | CB | GLN | C | 330 | 60.240 | 5.314 | −16.728 | 1.00 | 65.72 | | C |
| ANISOU | 2385 | CB | GLN | C | 330 | 9355 | 8898 | 6717 | −2120 | 945 | 339 | C |
| ATOM | 2386 | CG | GLN | C | 330 | 58.861 | 5.517 | −16.179 | 1.00 | 60.50 | | C |
| ANISOU | 2386 | CG | GLN | C | 330 | 8671 | 8401 | 5914 | −2317 | 968 | 207 | C |
| ATOM | 2387 | CD | GLN | C | 330 | 58.343 | 6.856 | −16.576 | 1.00 | 74.86 | | C |
| ANISOU | 2387 | CD | GLN | C | 330 | 10353 | 10407 | 7684 | −2166 | 1044 | 137 | C |
| ATOM | 2388 | NE2 | GLN | C | 330 | 57.300 | 6.864 | −17.379 | 1.00 | 67.67 | | N |
| ANISOU | 2388 | NE2 | GLN | C | 330 | 9373 | 9671 | 6667 | −2098 | 1092 | −29 | N |
| ATOM | 2389 | OE1 | GLN | C | 330 | 58.849 | 7.894 | −16.153 | 1.00 | 73.24 | | O |
| ANISOU | 2389 | OE1 | GLN | C | 330 | 10109 | 10183 | 7535 | −2103 | 1048 | 226 | O |
| ATOM | 2390 | N | SER | C | 331 | 61.832 | 2.731 | −18.065 | 1.00 | 71.11 | | N |
| ANISOU | 2390 | N | SER | C | 331 | 10212 | 9243 | 7565 | −1887 | 804 | 362 | N |
| ATOM | 2391 | CA | SER | C | 331 | 62.719 | 2.222 | −19.107 | 1.00 | 71.62 | | C |
| ANISOU | 2391 | CA | SER | C | 331 | 10249 | 9260 | 7703 | −1632 | 787 | 356 | C |
| ATOM | 2392 | C | SER | C | 331 | 62.015 | 1.089 | −19.839 | 1.00 | 76.58 | | C |
| ANISOU | 2392 | C | SER | C | 331 | 10950 | 9865 | 8281 | −1626 | 748 | 181 | C |
| ATOM | 2393 | O | SER | C | 331 | 60.964 | 0.607 | −19.411 | 1.00 | 76.40 | | O |
| ANISOU | 2393 | O | SER | C | 331 | 11024 | 9817 | 8188 | −1858 | 716 | 97 | O |
| ATOM | 2394 | CB | SER | C | 331 | 64.065 | 1.749 | −18.552 | 1.00 | 76.05 | | C |
| ANISOU | 2394 | CB | SER | C | 331 | 10880 | 9627 | 8389 | −1584 | 678 | 483 | C |
| ATOM | 2395 | OG | SER | C | 331 | 65.054 | 2.767 | −18.581 | 1.00 | 84.55 | | O |
| ANISOU | 2395 | OG | SER | C | 331 | 11822 | 10772 | 9532 | −1463 | 733 | 614 | O |
| ATOM | 2396 | N | LEU | C | 332 | 62.591 | 0.693 | −20.961 | 1.00 | 73.70 | | N |
| ANISOU | 2396 | N | LEU | C | 332 | 10528 | 9529 | 7945 | −1368 | 753 | 118 | N |
| ATOM | 2397 | CA | LEU | C | 332 | 62.089 | −0.352 | −21.819 | 1.00 | 74.72 | | C |
| ANISOU | 2397 | CA | LEU | C | 332 | 10710 | 9639 | 8040 | −1306 | 708 | −68 | C |
| ATOM | 2398 | C | LEU | C | 332 | 63.226 | −1.364 | −21.901 | 1.00 | 81.83 | | C |
| ANISOU | 2398 | C | LEU | C | 332 | 11709 | 10326 | 9057 | −1161 | 572 | −58 | C |
| ATOM | 2399 | O | LEU | C | 332 | 64.365 | −0.980 | −22.193 | 1.00 | 82.05 | | O |
| ANISOU | 2399 | O | LEU | C | 332 | 11631 | 10399 | 9144 | −948 | 592 | 28 | O |
| ATOM | 2400 | CB | LEU | C | 332 | 61.802 | 0.300 | −23.185 | 1.00 | 74.00 | | C |
| ANISOU | 2400 | CB | LEU | C | 332 | 10437 | 9819 | 7862 | −1072 | 836 | −161 | C |
| ATOM | 2401 | CG | LEU | C | 332 | 60.510 | −0.034 | −23.928 | 1.00 | 78.29 | | C |
| ANISOU | 2401 | CG | LEU | C | 332 | 10959 | 10503 | 8286 | −1101 | 866 | −371 | C |
| ATOM | 2402 | CD1 | LEU | C | 332 | 59.307 | −0.009 | −23.022 | 1.00 | 78.20 | | C |
| ANISOU | 2402 | CD1 | LEU | C | 332 | 11013 | 10488 | 8210 | −1421 | 859 | −413 | C |
| ATOM | 2403 | CD2 | LEU | C | 332 | 60.300 | 0.922 | −25.073 | 1.00 | 78.34 | | C |
| ANISOU | 2403 | CD2 | LEU | C | 332 | 10782 | 10795 | 8190 | −877 | 999 | −400 | C |
| ATOM | 2404 | N | HIS | C | 333 | 62.956 | −2.627 | −21.545 | 1.00 | 80.34 | | N |
| ANISOU | 2404 | N | HIS | C | 333 | 11727 | 9897 | 8902 | −1290 | 421 | −134 | N |
| ATOM | 2405 | CA | HIS | C | 333 | 63.996 | −3.662 | −21.551 | 1.00 | 82.26 | | C |
| ANISOU | 2405 | CA | HIS | C | 333 | 12096 | 9898 | 9260 | −1135 | 253 | −140 | C |
| ATOM | 2406 | C | HIS | C | 333 | 63.650 | −4.811 | −22.481 | 1.00 | 86.90 | | C |
| ANISOU | 2406 | C | HIS | C | 333 | 12771 | 10395 | 9851 | −1025 | 158 | −362 | C |
| ATOM | 2407 | O | HIS | C | 333 | 62.478 | −5.171 | −22.564 | 1.00 | 86.99 | | O |
| ANISOU | 2407 | O | HIS | C | 333 | 12861 | 10394 | 9798 | −1228 | 158 | −474 | O |
| ATOM | 2408 | CB | HIS | C | 333 | 64.210 | −4.240 | −20.140 | 1.00 | 84.22 | | C |
| ANISOU | 2408 | CB | HIS | C | 333 | 12577 | 9853 | 9571 | −1371 | 100 | −2 | C |
| ATOM | 2409 | CG | HIS | C | 333 | 64.501 | −3.251 | −19.051 | 1.00 | 86.34 | | C |
| ANISOU | 2409 | CG | HIS | C | 333 | 12791 | 10177 | 9836 | −1508 | 159 | 200 | C |
| ATOM | 2410 | CD2 | HIS | C | 333 | 64.758 | −1.924 | −19.113 | 1.00 | 86.45 | | C |
| ANISOU | 2410 | CD2 | HIS | C | 333 | 12593 | 10431 | 9824 | −1453 | 316 | 281 | C |
| ATOM | 2411 | ND1 | HIS | C | 333 | 64.570 | −3.653 | −17.730 | 1.00 | 88.76 | | N |
| ANISOU | 2411 | ND1 | HIS | C | 333 | 13293 | 10263 | 10169 | −1727 | 32 | 335 | N |
| ATOM | 2412 | CE1 | HIS | C | 333 | 64.855 | −2.566 | −17.037 | 1.00 | 86.70 | | C |
| ANISOU | 2412 | CE1 | HIS | C | 333 | 12916 | 10132 | 9894 | −1789 | 119 | 473 | C |
| ATOM | 2413 | NE2 | HIS | C | 333 | 64.974 | −1.503 | −17.828 | 1.00 | 85.62 | | N |
| ANISOU | 2413 | NE2 | HIS | C | 333 | 12540 | 10255 | 9735 | −1633 | 285 | 445 | N |
| ATOM | 2414 | N | THR | C | 334 | 64.665 | −5.417 | −23.139 | 1.00 | 84.02 | | N |
| ANISOU | 2414 | N | THR | C | 334 | 12392 | 9971 | 9562 | −711 | 66 | −442 | N |
| ATOM | 2415 | CA | THR | C | 334 | 64.475 | −6.588 | −24.005 | 1.00 | 86.09 | | C |
| ANISOU | 2415 | CA | THR | C | 334 | 12750 | 10118 | 9843 | −568 | −57 | −677 | C |
| ATOM | 2416 | C | THR | C | 334 | 65.775 | −7.342 | −24.283 | 1.00 | 92.33 | | C |
| ANISOU | 2416 | C | THR | C | 334 | 13575 | 10765 | 10741 | −243 | −218 | −738 | C |
| ATOM | 2417 | O | THR | C | 334 | 66.779 | −6.756 | −24.679 | 1.00 | 90.74 | | O |
| ANISOU | 2417 | O | THR | C | 334 | 13167 | 10769 | 10543 | 17 | −143 | −697 | O |
| ATOM | 2418 | CB | THR | C | 334 | 63.692 | −6.312 | −25.320 | 1.00 | 92.33 | | C |
| ANISOU | 2418 | CB | THR | C | 334 | 13363 | 11201 | 10515 | −453 | 80 | −870 | C |
| ATOM | 2419 | CG2 | THR | C | 334 | 64.217 | −5.121 | −26.115 | 1.00 | 87.96 | | C |
| ANISOU | 2419 | CG2 | THR | C | 334 | 12517 | 11017 | 9886 | −205 | 272 | −815 | C |
| ATOM | 2420 | OG1 | THR | C | 334 | 63.704 | −7.499 | −26.123 | 1.00 | 94.20 | | O |
| ANISOU | 2420 | OG1 | THR | C | 334 | 13696 | 11311 | 10786 | −283 | −65 | −1112 | O |
| ATOM | 2421 | N | THR | C | 335 | 65.714 | −8.667 | −24.094 | 1.00 | 92.33 | | N |
| ANISOU | 2421 | N | THR | C | 335 | 13843 | 10415 | 10825 | −264 | −448 | −850 | N |
| ATOM | 2422 | CA | THR | C | 335 | 66.802 | −9.604 | −24.346 | 1.00 | 94.74 | | C |
| ANISOU | 2422 | CA | THR | C | 335 | 14232 | 10527 | 11237 | 57 | −656 | −962 | C |
| ATOM | 2423 | C | THR | C | 335 | 66.553 | −10.099 | −25.759 | 1.00 | 100.87 | | C |
| ANISOU | 2423 | C | THR | C | 335 | 14919 | 11435 | 11974 | 310 | −656 | −1257 | C |
| ATOM | 2424 | O | THR | C | 335 | 65.429 | −10.499 | −26.068 | 1.00 | 100.52 | | O |
| ANISOU | 2424 | O | THR | C | 335 | 14970 | 11337 | 11887 | 128 | −659 | −1384 | O |
| ATOM | 2425 | CB | THR | C | 335 | 66.832 | −10.735 | −23.291 | 1.00 | 104.48 | | C |
| ANISOU | 2425 | CB | THR | C | 335 | 15846 | 11269 | 12583 | −115 | −929 | −904 | C |
| ATOM | 2426 | CG2 | THR | C | 335 | 68.244 | −11.181 | −22.958 | 1.00 | 105.52 | | C |
| ANISOU | 2426 | CG2 | THR | C | 335 | 16032 | 11233 | 12826 | 193 | −1124 | −887 | C |
| ATOM | 2427 | OG1 | THR | C | 335 | 66.179 | −10.324 | −22.087 | 1.00 | 101.48 | | O |
| ANISOU | 2427 | OG1 | THR | C | 335 | 15582 | 10805 | 12169 | −527 | −878 | −667 | O |
| ATOM | 2428 | N | VAL | C | 336 | 67.566 | −9.983 | −26.638 | 1.00 | 99.65 | | N |
| ANISOU | 2428 | N | VAL | C | 336 | 14546 | 11505 | 11811 | 717 | −634 | −1371 | N |
| ATOM | 2429 | CA | VAL | C | 336 | 67.483 | −10.352 | −28.063 | 1.00 | 101.50 | | C |
| ANISOU | 2429 | CA | VAL | C | 336 | 14644 | 11941 | 11979 | 1013 | −618 | −1661 | C |
| ATOM | 2430 | C | VAL | C | 336 | 68.666 | −11.206 | −28.525 | 1.00 | 109.65 | | C |
| ANISOU | 2430 | C | VAL | C | 336 | 15669 | 12904 | 13088 | 1434 | −809 | −1854 | C |
| ATOM | 2431 | O | VAL | C | 336 | 69.779 | −11.023 | −28.034 | 1.00 | 109.27 | | O |
| ANISOU | 2431 | O | VAL | C | 336 | 15556 | 12867 | 13097 | 1579 | −849 | −1740 | O |
| ATOM | 2432 | CB | VAL | C | 336 | 67.273 | −9.130 | −29.003 | 1.00 | 103.38 | | C |
| ANISOU | 2432 | CB | VAL | C | 336 | 14542 | 12687 | 12050 | 1091 | −326 | −1641 | C |
| ATOM | 2433 | CG1 | VAL | C | 336 | 65.873 | −8.543 | −28.854 | 1.00 | 101.14 | | C |
| ANISOU | 2433 | CG1 | VAL | C | 336 | 14281 | 12471 | 11678 | 747 | −184 | −1573 | C |
| ATOM | 2434 | CG2 | VAL | C | 336 | 68.345 | −8.054 | −28.797 | 1.00 | 101.76 | | C |
| ANISOU | 2434 | CG2 | VAL | C | 336 | 14101 | 12740 | 11825 | 1195 | −178 | −1424 | C |
| ATOM | 2435 | N | ILE | C | 337 | 68.433 | −12.114 | −29.495 | 1.00 | 109.73 | | N |
| ANISOU | 2435 | N | ILE | C | 337 | 15725 | 12875 | 13094 | 1649 | −930 | −2170 | N |
| ATOM | 2436 | CA | ILE | C | 337 | 69.494 | −12.983 | −30.013 | 1.00 | 112.71 | | C |
| ANISOU | 2436 | CA | ILE | C | 337 | 16088 | 13203 | 13535 | 2088 | −1129 | −2409 | C |
| ATOM | 2437 | C | ILE | C | 337 | 70.316 | −12.216 | −31.058 | 1.00 | 118.08 | | C |
| ANISOU | 2437 | C | ILE | C | 337 | 16352 | 14438 | 14074 | 2433 | −926 | −2478 | C |
| ATOM | 2438 | O | ILE | C | 337 | 69.913 | −12.090 | −32.218 | 1.00 | 117.95 | | O |
| ANISOU | 2438 | O | ILE | C | 337 | 16163 | 14725 | 13927 | 2578 | −815 | −2670 | O |
| ATOM | 2439 | CB | ILE | C | 337 | 69.025 | −14.393 | −30.488 | 1.00 | 118.56 | | C |
| ANISOU | 2439 | CB | ILE | C | 337 | 17088 | 13606 | 14354 | 2195 | −1397 | −2734 | C |
| ATOM | 2440 | CG1 | ILE | C | 337 | 67.724 | −14.857 | −29.786 | 1.00 | 118.50 | | C |
| ANISOU | 2440 | CG1 | ILE | C | 337 | 17426 | 13189 | 14410 | 1730 | −1487 | −2660 | C |
| ATOM | 2441 | CG2 | ILE | C | 337 | 70.149 | −15.405 | −30.270 | 1.00 | 122.22 | | C |
| ANISOU | 2441 | CG2 | ILE | C | 337 | 17692 | 13793 | 14956 | 2546 | −1699 | −2880 | C |
| ATOM | 2442 | CD1 | ILE | C | 337 | 66.814 | −15.771 | −30.626 | 1.00 | 127.17 | | C |
| ANISOU | 2442 | CD1 | ILE | C | 337 | 18662 | 14146 | 15512 | 1734 | −1613 | −2983 | C |
| ATOM | 2443 | N | ALA | C | 338 | 71.453 | −11.665 | −30.599 | 1.00 | 115.65 | | N |
| ANISOU | 2443 | N | ALA | C | 338 | 15882 | 14276 | 13783 | 2535 | −872 | −2302 | N |
| ATOM | 2444 | CA | ALA | C | 338 | 72.412 | −10.859 | −31.355 | 1.00 | 116.23 | | C |
| ANISOU | 2444 | CA | ALA | C | 338 | 15559 | 14872 | 13731 | 2804 | −676 | −2300 | C |
| ATOM | 2445 | C | ALA | C | 338 | 73.689 | −11.634 | −31.728 | 1.00 | 125.91 | | C |
| ANISOU | 2445 | C | ALA | C | 338 | 16688 | 16153 | 15001 | 3260 | −862 | −2540 | C |
| ATOM | 2446 | O | ALA | C | 338 | 73.984 | −12.667 | −31.124 | 1.00 | 127.81 | | O |
| ANISOU | 2446 | O | ALA | C | 338 | 17193 | 15973 | 15394 | 3354 | −1157 | −2645 | O |
| ATOM | 2447 | CB | ALA | C | 338 | 72.781 | −9.625 | −30.544 | 1.00 | 114.19 | | C |
| ANISOU | 2447 | CB | ALA | C | 338 | 15172 | 14750 | 13465 | 2568 | −488 | −1943 | C |
| ATOM | 2448 | N | LEU | C | 339 | 74.441 | −11.115 | −32.732 | 1.00 | 124.67 | | N |
| ANISOU | 2448 | N | LEU | C | 339 | 16147 | 16529 | 14694 | 3545 | −692 | −2627 | N |
| ATOM | 2449 | CA | LEU | C | 339 | 75.726 | −11.618 | −33.263 | 1.00 | 128.18 | | C |
| ANISOU | 2449 | CA | LEU | C | 339 | 16381 | 17198 | 15124 | 4007 | −804 | −2868 | C |
| ATOM | 2450 | C | LEU | C | 339 | 75.791 | −13.161 | −33.412 | 1.00 | 135.99 | | C |
| ANISOU | 2450 | C | LEU | C | 339 | 17613 | 17830 | 16228 | 4317 | −1157 | −3238 | C |
| ATOM | 2451 | O | LEU | C | 339 | 76.818 | −13.781 | −33.106 | 1.00 | 137.56 | | O |
| ANISOU | 2451 | O | LEU | C | 339 | 17818 | 17931 | 16520 | 4615 | −1375 | −3370 | O |
| ATOM | 2452 | CB | LEU | C | 339 | 76.933 | −11.085 | −32.442 | 1.00 | 128.03 | | C |
| ANISOU | 2452 | CB | LEU | C | 339 | 16228 | 17245 | 15170 | 4013 | −799 | −2661 | C |
| ATOM | 2453 | CG | LEU | C | 339 | 76.783 | −9.780 | −31.627 | 1.00 | 129.03 | | C |
| ANISOU | 2453 | CG | LEU | C | 339 | 16287 | 17454 | 15284 | 3607 | −561 | −2249 | C |
| ATOM | 2454 | CD1 | LEU | C | 339 | 77.904 | −9.657 | −30.632 | 1.00 | 129.52 | | C |
| ANISOU | 2454 | CD1 | LEU | C | 339 | 16330 | 17417 | 15463 | 3615 | −665 | −2105 | C |
| ATOM | 2455 | CD2 | LEU | C | 339 | 76.717 | −8.532 | −32.522 | 1.00 | 129.65 | | C |
| ANISOU | 2455 | CD2 | LEU | C | 339 | 16000 | 18105 | 15156 | 3567 | −216 | −2140 | C |
| ATOM | 2456 | N | GLU | C | 340 | 74.680 | −13.749 | −33.921 | 1.00 | 133.72 | | N |
| ANISOU | 2456 | N | GLU | C | 340 | 17516 | 17363 | 15929 | 4254 | −1215 | −3416 | N |
| ATOM | 2457 | CA | GLU | C | 340 | 74.421 | −15.178 | −34.153 | 1.00 | 137.03 | | C |
| ANISOU | 2457 | CA | GLU | C | 340 | 18211 | 17399 | 16456 | 4473 | −1540 | −3770 | C |
| ATOM | 2458 | C | GLU | C | 340 | 74.638 | −16.057 | −32.897 | 1.00 | 142.01 | | C |
| ANISOU | 2458 | C | GLU | C | 340 | 19249 | 17386 | 17322 | 4409 | −1879 | −3716 | C |
| ATOM | 2459 | O | GLU | C | 340 | 75.777 | −16.345 | −32.511 | 1.00 | 143.91 | | O |
| ANISOU | 2459 | O | GLU | C | 340 | 19457 | 17584 | 17637 | 4704 | −2053 | −3777 | O |
| ATOM | 2460 | CB | GLU | C | 340 | 75.183 | −15.726 | −35.371 | 1.00 | 141.97 | | C |
| ANISOU | 2460 | CB | GLU | C | 340 | 18574 | 18390 | 16978 | 5017 | −1606 | −4178 | C |
| ATOM | 2461 | CG | GLU | C | 340 | 74.375 | −15.630 | −36.654 | 1.00 | 154.80 | | C |
| ANISOU | 2461 | CG | GLU | C | 340 | 20149 | 20200 | 18470 | 5084 | −1540 | −4444 | C |
| ATOM | 2462 | CD | GLU | C | 340 | 74.783 | −16.599 | −37.748 | 1.00 | 184.23 | | C |
| ANISOU | 2462 | CD | GLU | C | 340 | 23742 | 24121 | 22137 | 5620 | −1708 | −4926 | C |
| ATOM | 2463 | OE1 | GLU | C | 340 | 73.942 | −17.438 | −38.143 | 1.00 | 180.66 | | O |
| ANISOU | 2463 | OE1 | GLU | C | 340 | 23466 | 23476 | 21701 | 5674 | −1853 | −5212 | O |
| ATOM | 2464 | OE2 | GLU | C | 340 | 75.941 | −16.515 | −38.217 | 1.00 | 183.59 | | O |
| ANISOU | 2464 | OE2 | GLU | C | 340 | 23365 | 24409 | 21984 | 5985 | −1695 | −5034 | O |
| ATOM | 2465 | N | GLY | C | 341 | 73.518 | −16.456 | −32.286 | 1.00 | 136.62 | | N |
| ANISOU | 2465 | N | GLY | C | 341 | 18940 | 16230 | 16739 | 4018 | −1968 | −3599 | N |
| ATOM | 2466 | CA | GLY | C | 341 | 73.452 | −17.309 | −31.102 | 1.00 | 137.05 | | C |
| ANISOU | 2466 | CA | GLY | C | 341 | 19441 | 15636 | 16995 | 3864 | −2280 | −3509 | C |
| ATOM | 2467 | C | GLY | C | 341 | 73.920 | −16.697 | −29.795 | 1.00 | 136.80 | | C |
| ANISOU | 2467 | C | GLY | C | 341 | 19464 | 15490 | 17023 | 3634 | −2249 | −3131 | C |
| ATOM | 2468 | O | GLY | C | 341 | 74.550 | −17.393 | −28.991 | 1.00 | 138.77 | | O |
| ANISOU | 2468 | O | GLY | C | 341 | 19963 | 15349 | 17413 | 3740 | −2537 | −3112 | O |
| ATOM | 2469 | N | LEU | C | 342 | 73.616 | −15.399 | −29.556 | 1.00 | 127.33 | | N |
| ANISOU | 2469 | N | LEU | C | 342 | 18045 | 14617 | 15717 | 3328 | −1919 | −2832 | N |
| ATOM | 2470 | CA | LEU | C | 342 | 74.011 | −14.712 | −28.319 | 1.00 | 124.25 | | C |
| ANISOU | 2470 | CA | LEU | C | 342 | 17683 | 14155 | 15373 | 3090 | −1869 | −2477 | C |
| ATOM | 2471 | C | LEU | C | 342 | 72.897 | −13.815 | −27.741 | 1.00 | 123.11 | | C |
| ANISOU | 2471 | C | LEU | C | 342 | 17587 | 14017 | 15173 | 2564 | −1635 | −2173 | C |
| ATOM | 2472 | O | LEU | C | 342 | 72.526 | −12.834 | −28.383 | 1.00 | 120.30 | | O |
| ANISOU | 2472 | O | LEU | C | 342 | 16949 | 14078 | 14681 | 2487 | −1340 | −2125 | O |
| ATOM | 2473 | CB | LEU | C | 342 | 75.303 | −13.883 | −28.502 | 1.00 | 123.73 | | C |
| ANISOU | 2473 | CB | LEU | C | 342 | 17212 | 14559 | 15240 | 3357 | −1725 | −2430 | C |
| ATOM | 2474 | CG | LEU | C | 342 | 76.585 | −14.615 | −28.869 | 1.00 | 131.77 | | C |
| ANISOU | 2474 | CG | LEU | C | 342 | 18139 | 15618 | 16309 | 3873 | −1957 | −2693 | C |
| ATOM | 2475 | CD1 | LEU | C | 342 | 77.421 | −13.780 | −29.801 | 1.00 | 131.89 | | C |
| ANISOU | 2475 | CD1 | LEU | C | 342 | 17650 | 16290 | 16172 | 4153 | −1719 | −2788 | C |
| ATOM | 2476 | CD2 | LEU | C | 342 | 77.374 | −15.003 | −27.631 | 1.00 | 134.62 | | C |
| ANISOU | 2476 | CD2 | LEU | C | 342 | 18694 | 15648 | 16806 | 3886 | −2192 | −2542 | C |
| ATOM | 2477 | N | PRO | C | 343 | 72.386 | −14.085 | −26.516 | 1.00 | 118.36 | | N |
| ANISOU | 2477 | N | PRO | C | 343 | 17328 | 12981 | 14662 | 2206 | −1759 | −1958 | N |
| ATOM | 2478 | CA | PRO | C | 343 | 71.360 | −13.192 | −25.940 | 1.00 | 114.47 | | C |
| ANISOU | 2478 | CA | PRO | C | 343 | 16847 | 12543 | 14101 | 1725 | −1533 | −1689 | C |
| ATOM | 2479 | C | PRO | C | 343 | 71.944 | −11.847 | −25.481 | 1.00 | 113.80 | | C |
| ANISOU | 2479 | C | PRO | C | 343 | 16470 | 12813 | 13955 | 1662 | −1294 | −1436 | C |
| ATOM | 2480 | O | PRO | C | 343 | 73.062 | −11.807 | −24.955 | 1.00 | 114.55 | | O |
| ANISOU | 2480 | O | PRO | C | 343 | 16487 | 12934 | 14103 | 1860 | −1375 | −1378 | O |
| ATOM | 2481 | CB | PRO | C | 343 | 70.790 | −14.007 | −24.779 | 1.00 | 117.50 | | C |
| ANISOU | 2481 | CB | PRO | C | 343 | 17672 | 12384 | 14588 | 1405 | −1761 | −1551 | C |
| ATOM | 2482 | CG | PRO | C | 343 | 71.886 | −14.922 | −24.379 | 1.00 | 125.25 | | C |
| ANISOU | 2482 | CG | PRO | C | 343 | 18821 | 13074 | 15694 | 1719 | −2073 | −1617 | C |
| ATOM | 2483 | CD | PRO | C | 343 | 72.717 | −15.198 | −25.600 | 1.00 | 122.75 | | C |
| ANISOU | 2483 | CD | PRO | C | 343 | 18275 | 12994 | 15369 | 2228 | −2114 | −1947 | C |
| ATOM | 2484 | N | LEU | C | 344 | 71.191 | −10.747 | −25.687 | 1.00 | 105.22 | | N |
| ANISOU | 2484 | N | LEU | C | 344 | 15222 | 11997 | 12759 | 1396 | −1012 | −1298 | N |
| ATOM | 2485 | CA | LEU | C | 344 | 71.630 | −9.387 | −25.363 | 1.00 | 101.43 | | C |
| ANISOU | 2485 | CA | LEU | C | 344 | 14475 | 11842 | 12220 | 1314 | −779 | −1067 | C |
| ATOM | 2486 | C | LEU | C | 344 | 70.482 | −8.496 | −24.861 | 1.00 | 100.46 | | C |
| ANISOU | 2486 | C | LEU | C | 344 | 14381 | 11757 | 12031 | 893 | −591 | −865 | C |
| ATOM | 2487 | O | LEU | C | 344 | 69.519 | −8.255 | −25.587 | 1.00 | 98.82 | | O |
| ANISOU | 2487 | O | LEU | C | 344 | 14116 | 11701 | 11729 | 806 | −454 | −946 | O |
| ATOM | 2488 | CB | LEU | C | 344 | 72.329 | −8.779 | −26.603 | 1.00 | 101.44 | | C |
| ANISOU | 2488 | CB | LEU | C | 344 | 14094 | 12325 | 12123 | 1636 | −599 | −1182 | C |
| ATOM | 2489 | CG | LEU | C | 344 | 72.674 | −7.294 | −26.592 | 1.00 | 103.32 | | C |
| ANISOU | 2489 | CG | LEU | C | 344 | 14040 | 12944 | 12275 | 1531 | −320 | −965 | C |
| ATOM | 2490 | CD1 | LEU | C | 344 | 74.169 | −7.084 | −26.532 | 1.00 | 104.30 | | C |
| ANISOU | 2490 | CD1 | LEU | C | 344 | 13942 | 13258 | 12429 | 1753 | −324 | −917 | C |
| ATOM | 2491 | CD2 | LEU | C | 344 | 72.122 | −6.623 | −27.817 | 1.00 | 104.72 | | C |
| ANISOU | 2491 | CD2 | LEU | C | 344 | 14002 | 13489 | 12298 | 1596 | −99 | −1048 | C |
| ATOM | 2492 | N | GLU | C | 345 | 70.601 | −7.997 | −23.622 | 1.00 | 94.96 | | N |
| ANISOU | 2492 | N | GLU | C | 345 | 13762 | 10945 | 11374 | 651 | −592 | −620 | N |
| ATOM | 2493 | CA | GLU | C | 345 | 69.599 | −7.109 | −23.031 | 1.00 | 91.75 | | C |
| ANISOU | 2493 | CA | GLU | C | 345 | 13369 | 10592 | 10902 | 272 | −426 | −440 | C |
| ATOM | 2494 | C | GLU | C | 345 | 69.816 | −5.693 | −23.534 | 1.00 | 92.33 | | C |
| ANISOU | 2494 | C | GLU | C | 345 | 13115 | 11075 | 10892 | 319 | −164 | −351 | C |
| ATOM | 2495 | O | GLU | C | 345 | 70.963 | −5.267 | −23.692 | 1.00 | 92.11 | | O |
| ANISOU | 2495 | O | GLU | C | 345 | 12887 | 11226 | 10885 | 531 | −129 | −312 | O |
| ATOM | 2496 | CB | GLU | C | 345 | 69.646 | −7.143 | −21.494 | 1.00 | 92.68 | | C |
| ANISOU | 2496 | CB | GLU | C | 345 | 13690 | 10447 | 11076 | 5 | −535 | −225 | C |
| ATOM | 2497 | N | VAL | C | 346 | 68.717 | −4.977 | −23.831 | 1.00 | 85.90 | | N |
| ANISOU | 2497 | N | VAL | C | 346 | 12245 | 10417 | 9978 | 127 | 11 | −328 | N |
| ATOM | 2498 | CA | VAL | C | 346 | 68.782 | −3.592 | −24.298 | 1.00 | 83.08 | | C |
| ANISOU | 2498 | CA | VAL | C | 346 | 11623 | 10408 | 9535 | 145 | 247 | −226 | C |
| ATOM | 2499 | C | VAL | C | 346 | 67.886 | −2.697 | −23.438 | 1.00 | 83.14 | | C |
| ANISOU | 2499 | C | VAL | C | 346 | 11676 | 10407 | 9507 | −189 | 346 | −66 | C |
| ATOM | 2500 | O | VAL | C | 346 | 66.689 | −2.948 | −23.307 | 1.00 | 81.83 | | O |
| ANISOU | 2500 | O | VAL | C | 346 | 11632 | 10172 | 9287 | −384 | 346 | −130 | O |
| ATOM | 2501 | CB | VAL | C | 346 | 68.554 | −3.370 | −25.824 | 1.00 | 86.97 | | C |
| ANISOU | 2501 | CB | VAL | C | 346 | 11926 | 11205 | 9912 | 364 | 382 | −379 | C |
| ATOM | 2502 | CG1 | VAL | C | 346 | 69.121 | −2.022 | −26.267 | 1.00 | 85.48 | | C |
| ANISOU | 2502 | CG1 | VAL | C | 346 | 11471 | 11350 | 9656 | 437 | 587 | −232 | C |
| ATOM | 2503 | CG2 | VAL | C | 346 | 69.147 | −4.498 | −26.669 | 1.00 | 89.28 | | C |
| ANISOU | 2503 | CG2 | VAL | C | 346 | 12216 | 11480 | 10226 | 679 | 252 | −608 | C |
| ATOM | 2504 | N | GLN | C | 347 | 68.485 | −1.672 | −22.839 | 1.00 | 78.06 | | N |
| ANISOU | 2504 | N | GLN | C | 347 | 10925 | 9844 | 8889 | −252 | 422 | 126 | N |
| ATOM | 2505 | CA | GLN | C | 347 | 67.795 | −0.692 | −22.011 | 1.00 | 76.01 | | C |
| ANISOU | 2505 | CA | GLN | C | 347 | 10681 | 9601 | 8597 | −528 | 512 | 267 | C |
| ATOM | 2506 | C | GLN | C | 347 | 67.462 | 0.506 | −22.895 | 1.00 | 78.61 | | C |
| ANISOU | 2506 | C | GLN | C | 347 | 10810 | 10225 | 8834 | −466 | 712 | 293 | C |
| ATOM | 2507 | O | GLN | C | 347 | 68.327 | 0.940 | −23.650 | 1.00 | 79.39 | | O |
| ANISOU | 2507 | O | GLN | C | 347 | 10731 | 10504 | 8931 | −265 | 788 | 327 | O |
| ATOM | 2508 | CB | GLN | C | 347 | 68.726 | −0.243 | −20.876 | 1.00 | 77.02 | | C |
| ANISOU | 2508 | CB | GLN | C | 347 | 10809 | 9641 | 8813 | −614 | 462 | 447 | C |
| ATOM | 2509 | CG | GLN | C | 347 | 68.133 | −0.399 | −19.485 | 1.00 | 89.15 | | C |
| ANISOU | 2509 | CG | GLN | C | 347 | 12556 | 10953 | 10362 | −905 | 360 | 518 | C |
| ATOM | 2510 | CD | GLN | C | 347 | 69.168 | −0.937 | −18.546 | 1.00 | 100.41 | | C |
| ANISOU | 2510 | CD | GLN | C | 347 | 14066 | 12197 | 11888 | −878 | 198 | 607 | C |
| ATOM | 2511 | NE2 | GLN | C | 347 | 69.798 | −0.047 | −17.785 | 1.00 | 89.43 | | N |
| ANISOU | 2511 | NE2 | GLN | C | 347 | 12582 | 10865 | 10531 | −949 | 233 | 755 | N |
| ATOM | 2512 | OE1 | GLN | C | 347 | 69.447 | −2.143 | −18.529 | 1.00 | 95.53 | | O |
| ANISOU | 2512 | OE1 | GLN | C | 347 | 13593 | 11387 | 11319 | −771 | 30 | 533 | O |
| ATOM | 2513 | N | ILE | C | 348 | 66.223 | 1.030 | −22.834 | 1.00 | 72.79 | | N |
| ANISOU | 2513 | N | ILE | C | 348 | 10097 | 9550 | 8010 | −629 | 792 | 274 | N |
| ATOM | 2514 | CA | ILE | C | 348 | 65.834 | 2.213 | −23.620 | 1.00 | 71.20 | | C |
| ANISOU | 2514 | CA | ILE | C | 348 | 9737 | 9600 | 7715 | −564 | 959 | 310 | C |
| ATOM | 2515 | C | ILE | C | 348 | 65.279 | 3.280 | −22.662 | 1.00 | 75.34 | | C |
| ANISOU | 2515 | C | ILE | C | 348 | 10282 | 10113 | 8233 | −786 | 1005 | 426 | C |
| ATOM | 2516 | O | ILE | C | 348 | 64.299 | 2.994 | −21.970 | 1.00 | 74.79 | | O |
| ANISOU | 2516 | O | ILE | C | 348 | 10326 | 9962 | 8129 | −983 | 961 | 360 | O |
| ATOM | 2517 | CB | ILE | C | 348 | 64.878 | 1.866 | −24.804 | 1.00 | 73.82 | | C |
| ANISOU | 2517 | CB | ILE | C | 348 | 10043 | 10081 | 7925 | −450 | 1007 | 124 | C |
| ATOM | 2518 | CG1 | ILE | C | 348 | 65.579 | 0.962 | −25.840 | 1.00 | 75.25 | | C |
| ANISOU | 2518 | CG1 | ILE | C | 348 | 10168 | 10318 | 8106 | −183 | 971 | 3 | C |
| ATOM | 2519 | CG2 | ILE | C | 348 | 64.313 | 3.135 | −25.475 | 1.00 | 72.70 | | C |
| ANISOU | 2519 | CG2 | ILE | C | 348 | 9778 | 10176 | 7669 | −406 | 1156 | 173 | C |
| ATOM | 2520 | CD1 | ILE | C | 348 | 64.648 | 0.039 | −26.645 | 1.00 | 80.58 | | C |
| ANISOU | 2520 | CD1 | ILE | C | 348 | 10905 | 11004 | 8709 | −122 | 921 | −240 | C |
| ATOM | 2521 | N | ARG | C | 349 | 65.933 | 4.484 | −22.592 | 1.00 | 71.98 | | N |
| ANISOU | 2521 | N | ARG | C | 349 | 9743 | 9768 | 7837 | −766 | 1085 | 591 | N |
| ATOM | 2522 | CA | ARG | C | 349 | 65.551 | 5.609 | −21.704 | 1.00 | 70.79 | | C |
| ANISOU | 2522 | CA | ARG | C | 349 | 9604 | 9597 | 7695 | −944 | 1114 | 695 | C |
| ATOM | 2523 | C | ARG | C | 349 | 65.675 | 6.973 | −22.390 | 1.00 | 74.90 | | C |
| ANISOU | 2523 | C | ARG | C | 349 | 10004 | 10274 | 8181 | −854 | 1236 | 801 | C |
| ATOM | 2524 | O | ARG | C | 349 | 65.978 | 7.017 | −23.579 | 1.00 | 74.83 | | O |
| ANISOU | 2524 | O | ARG | C | 349 | 9901 | 10413 | 8118 | −670 | 1309 | 803 | O |
| ATOM | 2525 | CB | ARG | C | 349 | 66.343 | 5.585 | −20.370 | 1.00 | 69.83 | | C |
| ANISOU | 2525 | CB | ARG | C | 349 | 9537 | 9306 | 7690 | −1088 | 1024 | 807 | C |
| ATOM | 2526 | N | THR | C | 350 | 65.415 | 8.077 | −21.644 | 1.00 | 72.08 | | N |
| ANISOU | 2526 | N | THR | C | 350 | 9659 | 9881 | 7845 | −985 | 1247 | 887 | N |
| ATOM | 2527 | CA | THR | C | 350 | 65.472 | 9.487 | −22.088 | 1.00 | 72.79 | | C |
| ANISOU | 2527 | CA | THR | C | 350 | 9684 | 10054 | 7920 | −938 | 1331 | 1007 | C |
| ATOM | 2528 | C | THR | C | 350 | 66.169 | 10.351 | −20.994 | 1.00 | 80.05 | | C |
| ANISOU | 2528 | C | THR | C | 350 | 10606 | 10849 | 8961 | −1085 | 1295 | 1148 | C |
| ATOM | 2529 | O | THR | C | 350 | 67.149 | 11.071 | −21.224 | 1.00 | 79.01 | | O |
| ANISOU | 2529 | O | THR | C | 350 | 10398 | 10728 | 8895 | −1065 | 1337 | 1303 | O |
| ATOM | 2530 | CB | THR | C | 350 | 64.035 | 10.026 | −22.391 | 1.00 | 75.37 | | C |
| ANISOU | 2530 | CB | THR | C | 350 | 10045 | 10466 | 8125 | −920 | 1356 | 900 | C |
| ATOM | 2531 | CG2 | THR | C | 350 | 63.413 | 9.420 | −23.633 | 1.00 | 71.27 | | C |
| ANISOU | 2531 | CG2 | THR | C | 350 | 9499 | 10103 | 7477 | −756 | 1400 | 770 | C |
| ATOM | 2532 | OG1 | THR | C | 350 | 63.171 | 9.801 | −21.270 | 1.00 | 73.66 | | O |
| ANISOU | 2532 | OG1 | THR | C | 350 | 9907 | 10179 | 7901 | −1091 | 1285 | 789 | O |
| ATOM | 2533 | N | ARG | C | 351 | 65.568 | 10.262 | −19.809 | 1.00 | 80.06 | | N |
| ANISOU | 2533 | N | ARG | C | 351 | 10692 | 10751 | 8975 | −1247 | 1217 | 1079 | N |
| ATOM | 2534 | CA | ARG | C | 351 | 65.836 | 10.801 | −18.478 | 1.00 | 80.60 | | C |
| ANISOU | 2534 | CA | ARG | C | 351 | 10794 | 10702 | 9128 | −1415 | 1150 | 1135 | C |
| ATOM | 2535 | C | ARG | C | 351 | 65.117 | 9.744 | −17.596 | 1.00 | 84.88 | | C |
| ANISOU | 2535 | C | ARG | C | 351 | 11434 | 11196 | 9620 | −1548 | 1071 | 1006 | C |
| ATOM | 2536 | O | ARG | C | 351 | 65.493 | 9.525 | −16.442 | 1.00 | 83.90 | | O |
| ANISOU | 2536 | O | ARG | C | 351 | 11358 | 10972 | 9548 | −1689 | 991 | 1039 | O |
| ATOM | 2537 | CB | ARG | C | 351 | 65.161 | 12.183 | −18.294 | 1.00 | 81.72 | | C |
| ANISOU | 2537 | CB | ARG | C | 351 | 10946 | 10856 | 9249 | −1441 | 1166 | 1136 | C |
| ATOM | 2538 | CG | ARG | C | 351 | 65.974 | 13.187 | −17.477 | 1.00 | 98.79 | | C |
| ANISOU | 2538 | CG | ARG | C | 351 | 13096 | 12908 | 11533 | −1544 | 1124 | 1255 | C |
| ATOM | 2539 | CD | ARG | C | 351 | 66.089 | 12.845 | −15.989 | 1.00 | 117.12 | | C |
| ANISOU | 2539 | CD | ARG | C | 351 | 15463 | 15146 | 13891 | −1718 | 1027 | 1211 | C |
| ATOM | 2540 | NE | ARG | C | 351 | 67.277 | 12.034 | −15.684 | 1.00 | 128.88 | | N |
| ANISOU | 2540 | NE | ARG | C | 351 | 16927 | 16575 | 15467 | −1748 | 986 | 1295 | N |
| ATOM | 2541 | CZ | ARG | C | 351 | 67.458 | 11.345 | −14.559 | 1.00 | 138.03 | | C |
| ANISOU | 2541 | CZ | ARG | C | 351 | 18141 | 17667 | 16635 | −1870 | 893 | 1271 | C |
| ATOM | 2542 | NH1 | ARG | C | 351 | 66.526 | 11.350 | −13.611 | 1.00 | 125.34 | | N |
| ANISOU | 2542 | NH1 | ARG | C | 351 | 16611 | 16065 | 14947 | −2001 | 847 | 1170 | N |
| ATOM | 2543 | NH2 | ARG | C | 351 | 68.565 | 10.638 | −14.377 | 1.00 | 117.28 | | N |
| ANISOU | 2543 | NH2 | ARG | C | 351 | 15490 | 14986 | 14084 | −1856 | 841 | 1344 | N |
| ATOM | 2544 | N | GLU | C | 352 | 64.092 | 9.064 | −18.213 | 1.00 | 82.10 | | N |
| ANISOU | 2544 | N | GLU | C | 352 | 11112 | 10927 | 9156 | −1507 | 1095 | 864 | N |
| ATOM | 2545 | CA | GLU | C | 352 | 63.235 | 7.982 | −17.714 | 1.00 | 110.95 | | C |
| ANISOU | 2545 | CA | GLU | C | 352 | 14858 | 14562 | 12734 | −1641 | 1040 | 732 | C |
| ATOM | 2546 | C | GLU | C | 352 | 62.803 | 7.011 | −18.875 | 1.00 | 87.31 | | C |
| ANISOU | 2546 | C | GLU | C | 352 | 11867 | 11633 | 9675 | −1521 | 1066 | 617 | C |
| ATOM | 2547 | O | GLU | C | 352 | 61.954 | 7.305 | −19.746 | 1.00 | 21.13 | | O |
| ANISOU | 2547 | O | GLU | C | 352 | 3406 | 3348 | 1274 | −1390 | 1041 | 445 | O |
| ATOM | 2548 | CB | GLU | C | 352 | 62.013 | 8.543 | −16.952 | 1.00 | 112.26 | | C |
| ANISOU | 2548 | CB | GLU | C | 352 | 15037 | 14813 | 12803 | −1788 | 1039 | 627 | C |
| ATOM | 2549 | CG | GLU | C | 352 | 62.302 | 9.232 | −15.617 | 1.00 | 126.25 | | C |
| ANISOU | 2549 | CG | GLU | C | 352 | 16822 | 16528 | 14621 | −1929 | 991 | 693 | C |
| ATOM | 2550 | CD | GLU | C | 352 | 62.952 | 8.432 | −14.497 | 1.00 | 152.96 | | C |
| ANISOU | 2550 | CD | GLU | C | 352 | 20289 | 19777 | 18050 | −2096 | 898 | 766 | C |
| ATOM | 2551 | OE1 | GLU | C | 352 | 63.717 | 9.038 | −13.711 | 1.00 | 148.45 | | O |
| ANISOU | 2551 | OE1 | GLU | C | 352 | 19705 | 19144 | 17556 | −2141 | 859 | 859 | O |
| ATOM | 2552 | OE2 | GLU | C | 352 | 62.680 | 7.215 | −14.381 | 1.00 | 150.20 | | O |
| ANISOU | 2552 | OE2 | GLU | C | 352 | 20031 | 19378 | 17659 | −2186 | 854 | 730 | O |
| TER | 2553 | | GLU | C | 352 | | | | | | | |
| HETATM | 2554 | MN | MN | D | 1 | 79.870 | 13.212 | 6.932 | 1.00 | 70.50 | | Mn |
| HETATM | 2555 | MN | MN | D | 2 | 77.625 | 8.162 | 6.843 | 1.00 | 93.32 | | Mn |
| END | | | | | | | | | | | | |

TABLE 2
Rel-AMP-G4P_complex_closed_form crystal structure atomic coordinates:

| | | | | | | | | | | |
|---|---|---|---|---|---|---|---|---|---|---|
| CRYST1 | 120.739 | 50.346 | 86.057 | 90.00 | 111.00 | 90.00 | C | 1 | 2 | 1 |

| | | | | | | | | | | | | |
|---|---|---|---|---|---|---|---|---|---|---|---|---|
| ATOM | 1 | N | GLY | A | 7 | 124.467 | −67.290 | 89.791 | 1.00 | 79.20 | | N |
| ANISOU | 1 | N | GLY | A | 7 | 5554 | 12967 | 11571 | 1429 | 1096 | −861 | N |
| ATOM | 2 | CA | GLY | A | 7 | 124.368 | −66.028 | 90.512 | 1.00 | 79.50 | | C |
| ANISOU | 2 | CA | GLY | A | 7 | 5364 | 13259 | 11583 | 1066 | 1021 | −1314 | C |
| ATOM | 3 | C | GLY | A | 7 | 123.138 | −65.951 | 91.397 | 1.00 | 85.85 | | C |
| ANISOU | 3 | C | GLY | A | 7 | 6407 | 13940 | 12271 | 1118 | 950 | −1195 | C |
| ATOM | 4 | O | GLY | A | 7 | 122.969 | −66.802 | 92.278 | 1.00 | 87.06 | | O |
| ANISOU | 4 | O | GLY | A | 7 | 6551 | 14411 | 12119 | 1562 | 845 | −928 | O |
| ATOM | 5 | N | LEU | A | 8 | 122.245 | −64.938 | 91.149 | 1.00 | 81.20 | | N |
| ANISOU | 5 | N | LEU | A | 8 | 6058 | 12869 | 11926 | 699 | 1047 | −1357 | N |
| ATOM | 6 | CA | LEU | A | 8 | 120.966 | −64.681 | 91.843 | 1.00 | 79.70 | | C |
| ANISOU | 6 | CA | LEU | A | 8 | 6124 | 12474 | 11687 | 670 | 1011 | −1278 | C |
| ATOM | 7 | C | LEU | A | 8 | 120.057 | −65.929 | 91.779 | 1.00 | 82.53 | | C |
| ANISOU | 7 | C | LEU | A | 8 | 6862 | 12518 | 11978 | 1038 | 1014 | −763 | C |
| ATOM | 8 | O | LEU | A | 8 | 119.048 | −66.029 | 92.477 | 1.00 | 82.14 | | O |
| ANISOU | 8 | O | LEU | A | 8 | 7003 | 12377 | 11829 | 1117 | 976 | −637 | O |
| ATOM | 9 | CB | LEU | A | 8 | 120.272 | −63.487 | 91.169 | 1.00 | 79.33 | | C |
| ANISOU | 9 | CB | LEU | A | 8 | 6314 | 11865 | 11964 | 218 | 1196 | −1453 | C |
| ATOM | 10 | N | TRP | A | 9 | 120.452 | −66.873 | 90.921 | 1.00 | 77.83 | | N |
| ANISOU | 10 | N | TRP | A | 9 | 6367 | 11747 | 11456 | 1233 | 1101 | −503 | N |
| ATOM | 11 | CA | TRP | A | 9 | 119.809 | −68.137 | 90.667 | 1.00 | 77.50 | | C |
| ANISOU | 11 | CA | TRP | A | 9 | 6662 | 11373 | 11411 | 1530 | 1209 | −83 | C |
| ATOM | 12 | C | TRP | A | 9 | 119.676 | −68.962 | 91.916 | 1.00 | 81.97 | | C |
| ANISOU | 12 | C | TRP | A | 9 | 7211 | 12269 | 11665 | 1976 | 1165 | 153 | C |
| ATOM | 13 | O | TRP | A | 9 | 118.620 | −69.548 | 92.124 | 1.00 | 81.20 | | O |
| ANISOU | 13 | O | TRP | A | 9 | 7442 | 11836 | 11575 | 2078 | 1272 | 394 | O |
| ATOM | 14 | CB | TRP | A | 9 | 120.579 | −68.910 | 89.592 | 1.00 | 76.42 | | C |
| ANISOU | 14 | CB | TRP | A | 9 | 6542 | 11094 | 11399 | 1645 | 1340 | 62 | C |
| ATOM | 15 | CG | TRP | A | 9 | 120.027 | −70.277 | 89.374 | 1.00 | 77.53 | | C |
| ANISOU | 15 | CG | TRP | A | 9 | 7012 | 10894 | 11552 | 1935 | 1533 | 440 | C |
| ATOM | 16 | CD1 | TRP | A | 9 | 120.560 | −71.449 | 89.814 | 1.00 | 80.73 | | C |
| ANISOU | 16 | CD1 | TRP | A | 9 | 7407 | 11476 | 11790 | 2415 | 1644 | 734 | C |
| ATOM | 17 | CD2 | TRP | A | 9 | 118.733 | −70.595 | 88.855 | 1.00 | 77.15 | | C |
| ANISOU | 17 | CD2 | TRP | A | 9 | 7344 | 10304 | 11665 | 1778 | 1683 | 543 | C |
| ATOM | 18 | NE1 | TRP | A | 9 | 119.707 | −72.490 | 89.535 | 1.00 | 80.44 | | N |
| ANISOU | 18 | NE1 | TRP | A | 9 | 7761 | 10942 | 11860 | 2522 | 1915 | 1000 | N |
| ATOM | 19 | CE2 | TRP | A | 9 | 118.568 | −71.991 | 88.964 | 1.00 | 81.08 | | C |
| ANISOU | 19 | CE2 | TRP | A | 9 | 8058 | 10616 | 12134 | 2109 | 1923 | 851 | C |
| ATOM | 20 | CE3 | TRP | A | 9 | 117.714 | −69.841 | 88.264 | 1.00 | 77.94 | | C |
| ANISOU | 20 | CE3 | TRP | A | 9 | 7607 | 10086 | 11921 | 1407 | 1674 | 396 | C |
| ATOM | 21 | CZ2 | TRP | A | 9 | 117.435 | −72.649 | 88.487 | 1.00 | 79.56 | | C |
| ANISOU | 21 | CZ2 | TRP | A | 9 | 8208 | 9932 | 12088 | 1994 | 2157 | 933 | C |
| ATOM | 22 | CZ3 | TRP | A | 9 | 116.599 | −70.496 | 87.782 | 1.00 | 78.85 | | C |
| ANISOU | 22 | CZ3 | TRP | A | 9 | 8022 | 9809 | 12127 | 1348 | 1846 | 503 | C |
| ATOM | 23 | CH2 | TRP | A | 9 | 116.469 | −71.883 | 87.896 | 1.00 | 79.26 | | C |
| ANISOU | 23 | CH2 | TRP | A | 9 | 8256 | 9686 | 12172 | 1599 | 2086 | 729 | C |
| ATOM | 24 | N | ASN | A | 10 | 120.722 | −68.998 | 92.760 | 1.00 | 80.44 | | N |
| ANISOU | 24 | N | ASN | A | 10 | 6618 | 12771 | 11173 | 2254 | 1031 | 76 | N |
| ATOM | 25 | CA | ASN | A | 10 | 120.727 | −69.776 | 94.004 | 1.00 | 81.43 | | C |
| ANISOU | 25 | CA | ASN | A | 10 | 6685 | 13336 | 10919 | 2791 | 1003 | 344 | C |
| ATOM | 26 | C | ASN | A | 10 | 119.662 | −69.316 | 95.006 | 1.00 | 86.69 | | C |
| ANISOU | 26 | C | ASN | A | 10 | 7462 | 14011 | 11466 | 2713 | 921 | 294 | C |
| ATOM | 27 | O | ASN | A | 10 | 119.336 | −70.061 | 95.931 | 1.00 | 87.12 | | O |
| ANISOU | 27 | O | ASN | A | 10 | 7611 | 14238 | 11253 | 3154 | 971 | 600 | O |
| ATOM | 28 | CB | ASN | A | 10 | 122.122 | −69.815 | 94.623 | 1.00 | 85.54 | | C |
| ANISOU | 28 | CB | ASN | A | 10 | 6672 | 14744 | 11083 | 3128 | 854 | 231 | C |
| ATOM | 29 | CG | ASN | A | 10 | 123.160 | −70.419 | 93.694 | 1.00 | 120.73 | | C |
| ANISOU | 29 | CG | ASN | A | 10 | 11033 | 19203 | 15634 | 3287 | 959 | 343 | C |
| ATOM | 30 | OD1 | ASN | A | 10 | 123.983 | −69.711 | 93.098 | 1.00 | 119.91 | | O |
| ANISOU | 30 | OD1 | ASN | A | 10 | 10628 | 19277 | 15656 | 2963 | 878 | −22 | O |
| ATOM | 31 | ND2 | ASN | A | 10 | 123.141 | −71.743 | 93.537 | 1.00 | 111.64 | | N |
| ANISOU | 31 | ND2 | ASN | A | 10 | 10153 | 17816 | 14448 | 3779 | 1193 | 841 | N |
| ATOM | 32 | N | ARG | A | 11 | 119.097 | −68.111 | 94.785 | 1.00 | 83.29 | | N |
| ANISOU | 32 | N | ARG | A | 11 | 7054 | 13351 | 11242 | 2178 | 845 | −60 | N |
| ATOM | 33 | CA | ARG | A | 11 | 118.017 | −67.508 | 95.571 | 1.00 | 82.99 | | C |
| ANISOU | 33 | CA | ARG | A | 11 | 7141 | 13234 | 11157 | 2018 | 785 | −154 | C |
| ATOM | 34 | C | ARG | A | 11 | 116.664 | −68.012 | 95.014 | 1.00 | 84.11 | | C |
| ANISOU | 34 | C | ARG | A | 11 | 7788 | 12638 | 11530 | 1949 | 964 | 130 | C |
| ATOM | 35 | O | ARG | A | 11 | 115.748 | −68.328 | 95.788 | 1.00 | 83.85 | | O |
| ANISOU | 35 | O | ARG | A | 11 | 7946 | 12534 | 11379 | 2093 | 995 | 297 | O |
| ATOM | 36 | CB | ARG | A | 11 | 118.129 | −65.957 | 95.493 | 1.00 | 85.23 | | C |
| ANISOU | 36 | CB | ARG | A | 11 | 7216 | 13581 | 11587 | 1486 | 704 | −673 | C |
| ATOM | 37 | CG | ARG | A | 11 | 116.968 | −65.138 | 96.086 | 1.00 | 93.11 | | C |
| ANISOU | 37 | CG | ARG | A | 11 | 8378 | 14381 | 12618 | 1239 | 689 | −809 | C |
| ATOM | 38 | CD | ARG | A | 11 | 117.190 | −63.648 | 95.884 | 1.00 | 99.35 | | C |
| ANISOU | 38 | CD | ARG | A | 11 | 9001 | 15142 | 13605 | 731 | 728 | −1304 | C |
| ATOM | 39 | NE | ARG | A | 11 | 116.667 | −63.178 | 94.597 | 1.00 | 109.80 | | N |
| ANISOU | 39 | NE | ARG | A | 11 | 10633 | 15777 | 15310 | 441 | 915 | −1255 | N |
| ATOM | 40 | CZ | ARG | A | 11 | 116.646 | −61.902 | 94.209 | 1.00 | 127.67 | | C |
| ANISOU | 40 | CZ | ARG | A | 11 | 12892 | 17803 | 17812 | 35 | 1071 | −1573 | C |
| ATOM | 41 | NH1 | ARG | A | 11 | 117.127 | −60.950 | 95.001 | 1.00 | 120.10 | | N |
| ANISOU | 41 | NH1 | ARG | A | 11 | 11633 | 17197 | 16801 | −213 | 1084 | −2037 | N |
| ATOM | 42 | NH2 | ARG | A | 11 | 116.139 | −61.570 | 93.028 | 1.00 | 110.67 | | N |
| ANISOU | 42 | NH2 | ARG | A | 11 | 11034 | 15076 | 15942 | −111 | 1259 | −1440 | N |
| ATOM | 43 | N | LEU | A | 12 | 116.554 | −68.079 | 93.669 | 1.00 | 77.85 | | N |
| ANISOU | 43 | N | LEU | A | 12 | 7177 | 11355 | 11047 | 1723 | 1091 | 152 | N |
| ATOM | 44 | CA | LEU | A | 12 | 115.341 | −68.509 | 92.990 | 1.00 | 76.30 | | C |
| ANISOU | 44 | CA | LEU | A | 12 | 7371 | 10569 | 11051 | 1608 | 1259 | 322 | C |
| ATOM | 45 | C | LEU | A | 12 | 115.134 | −70.027 | 93.044 | 1.00 | 82.19 | | C |
| ANISOU | 45 | C | LEU | A | 12 | 8348 | 11125 | 11756 | 1962 | 1483 | 683 | C |
| ATOM | 46 | O | LEU | A | 12 | 114.034 | −70.455 | 93.371 | 1.00 | 82.88 | | O |
| ANISOU | 46 | O | LEU | A | 12 | 8691 | 10947 | 11850 | 1979 | 1611 | 808 | O |
| ATOM | 47 | CB | LEU | A | 12 | 115.291 | −67.964 | 91.549 | 1.00 | 75.18 | | C |
| ANISOU | 47 | CB | LEU | A | 12 | 7295 | 10076 | 11192 | 1262 | 1317 | 183 | C |
| ATOM | 48 | CG | LEU | A | 12 | 114.128 | −68.422 | 90.659 | 1.00 | 78.16 | | C |
| ANISOU | 48 | CG | LEU | A | 12 | 7989 | 9977 | 11733 | 1140 | 1481 | 295 | C |
| ATOM | 49 | CD1 | LEU | A | 12 | 112.785 | −67.952 | 91.178 | 1.00 | 78.03 | | C |
| ANISOU | 49 | CD1 | LEU | A | 12 | 8119 | 9843 | 11684 | 1017 | 1456 | 259 | C |
| ATOM | 50 | CD2 | LEU | A | 12 | 114.319 | −67.972 | 89.247 | 1.00 | 78.45 | | C |
| ANISOU | 50 | CD2 | LEU | A | 12 | 8030 | 9815 | 11963 | 910 | 1534 | 192 | C |
| ATOM | 51 | N | GLU | A | 13 | 116.179 | −70.827 | 92.752 | 1.00 | 79.42 | | N |
| ANISOU | 51 | N | GLU | A | 13 | 7912 | 10894 | 11371 | 2243 | 1578 | 840 | N |
| ATOM | 52 | CA | GLU | A | 13 | 116.166 | −72.298 | 92.723 | 1.00 | 79.82 | | C |
| ANISOU | 52 | CA | GLU | A | 13 | 8199 | 10710 | 11418 | 2610 | 1895 | 1196 | C |
| ATOM | 53 | C | GLU | A | 13 | 115.359 | −72.970 | 93.860 | 1.00 | 82.95 | | C |
| ANISOU | 53 | C | GLU | A | 13 | 8814 | 11054 | 11650 | 2910 | 2065 | 1454 | C |
| ATOM | 54 | O | GLU | A | 13 | 114.556 | −73.844 | 93.524 | 1.00 | 82.56 | | O |
| ANISOU | 54 | O | GLU | A | 13 | 9101 | 10497 | 11770 | 2886 | 2401 | 1596 | O |
| ATOM | 55 | CB | GLU | A | 13 | 117.606 | −72.856 | 92.672 | 1.00 | 82.01 | | C |
| ANISOU | 55 | CB | GLU | A | 13 | 8259 | 11331 | 11568 | 2998 | 1925 | 1345 | C |
| ATOM | 56 | CG | GLU | A | 13 | 117.736 | −74.309 | 92.231 | 1.00 | 99.94 | | C |
| ANISOU | 56 | CG | GLU | A | 13 | 10803 | 13230 | 13937 | 3316 | 2338 | 1686 | C |
| ATOM | 57 | CD | GLU | A | 13 | 117.452 | −75.343 | 93.301 | 1.00 | 140.89 | | C |
| ANISOU | 57 | CD | GLU | A | 13 | 16201 | 18405 | 18926 | 3846 | 2634 | 2086 | C |
| ATOM | 58 | OE1 | GLU | A | 13 | 118.185 | −75.374 | 94.316 | 1.00 | 148.26 | | O |
| ANISOU | 58 | OE1 | GLU | A | 13 | 16899 | 19932 | 19500 | 4330 | 2522 | 2259 | O |
| ATOM | 59 | OE2 | GLU | A | 13 | 116.486 | −76.120 | 93.125 | 1.00 | 145.79 | | O |
| ANISOU | 59 | OE2 | GLU | A | 13 | 17210 | 18447 | 19737 | 3782 | 3009 | 2210 | O |
| ATOM | 60 | N | PRO | A | 14 | 115.530 | −72.631 | 95.174 | 1.00 | 79.30 | | N |
| ANISOU | 60 | N | PRO | A | 14 | 8169 | 11099 | 10860 | 3180 | 1888 | 1497 | N |
| ATOM | 61 | CA | PRO | A | 14 | 114.748 | −73.327 | 96.217 | 1.00 | 78.47 | | C |
| ANISOU | 61 | CA | PRO | A | 14 | 8309 | 10911 | 10597 | 3501 | 2101 | 1786 | C |
| ATOM | 62 | C | PRO | A | 14 | 113.232 | −73.211 | 96.070 | 1.00 | 81.86 | | C |
| ANISOU | 62 | C | PRO | A | 14 | 9044 | 10819 | 11242 | 3119 | 2217 | 1688 | C |
| ATOM | 63 | O | PRO | A | 14 | 112.533 | −74.203 | 96.277 | 1.00 | 81.99 | | O |
| ANISOU | 63 | O | PRO | A | 14 | 9384 | 10448 | 11320 | 3275 | 2597 | 1927 | O |
| ATOM | 64 | CB | PRO | A | 14 | 115.237 | −72.693 | 97.527 | 1.00 | 80.10 | | C |
| ANISOU | 64 | CB | PRO | A | 14 | 8178 | 11868 | 10389 | 3771 | 1802 | 1740 | C |
| ATOM | 65 | CG | PRO | A | 14 | 116.517 | −72.073 | 97.213 | 1.00 | 85.26 | | C |
| ANISOU | 65 | CG | PRO | A | 14 | 8404 | 13033 | 10957 | 3730 | 1532 | 1500 | C |
| ATOM | 66 | CD | PRO | A | 14 | 116.438 | −71.638 | 95.785 | 1.00 | 81.10 | | C |
| ANISOU | 66 | CD | PRO | A | 14 | 7949 | 12028 | 10837 | 3201 | 1526 | 1255 | C |
| ATOM | 67 | N | ALA | A | 15 | 112.731 | −72.013 | 95.691 | 1.00 | 76.93 | | N |
| ANISOU | 67 | N | ALA | A | 15 | 8314 | 10179 | 10736 | 2632 | 1940 | 1334 | N |
| ATOM | 68 | CA | ALA | A | 15 | 111.310 | −71.709 | 95.476 | 1.00 | 75.45 | | C |
| ANISOU | 68 | CA | ALA | A | 15 | 8337 | 9611 | 10718 | 2266 | 1991 | 1197 | C |
| ATOM | 69 | C | ALA | A | 15 | 110.667 | −72.543 | 94.341 | 1.00 | 77.08 | | C |
| ANISOU | 69 | C | ALA | A | 15 | 8801 | 9278 | 11209 | 2064 | 2314 | 1203 | C |
| ATOM | 70 | O | ALA | A | 15 | 109.442 | −72.648 | 94.292 | 1.00 | 75.40 | | O |
| ANISOU | 70 | O | ALA | A | 15 | 8765 | 8791 | 11092 | 1847 | 2448 | 1125 | O |
| ATOM | 71 | CB | ALA | A | 15 | 111.150 | −70.223 | 95.189 | 1.00 | 76.06 | | C |
| ANISOU | 71 | CB | ALA | A | 15 | 8226 | 9826 | 10847 | 1877 | 1670 | 860 | C |
| ATOM | 72 | N | LEU | A | 16 | 111.505 | −73.125 | 93.442 | 1.00 | 73.66 | | N |
| ANISOU | 72 | N | LEU | A | 16 | 8353 | 8741 | 10894 | 2122 | 2445 | 1255 | N |
| ATOM | 73 | CA | LEU | A | 16 | 111.113 | −73.942 | 92.284 | 1.00 | 73.37 | | C |
| ANISOU | 73 | CA | LEU | A | 16 | 8505 | 8261 | 11112 | 1921 | 2766 | 1201 | C |
| ATOM | 74 | C | LEU | A | 16 | 111.056 | −75.449 | 92.589 | 1.00 | 80.52 | | C |
| ANISOU | 74 | C | LEU | A | 16 | 9691 | 8829 | 12073 | 2212 | 3285 | 1470 | C |
| ATOM | 75 | O | LEU | A | 16 | 110.906 | −76.253 | 91.655 | 1.00 | 80.58 | | O |
| ANISOU | 75 | O | LEU | A | 16 | 9846 | 8467 | 12303 | 2057 | 3625 | 1399 | O |
| ATOM | 76 | CB | LEU | A | 16 | 112.060 | −73.704 | 91.102 | 1.00 | 72.65 | | C |
| ANISOU | 76 | CB | LEU | A | 16 | 8252 | 8222 | 11129 | 1809 | 2656 | 1090 | C |
| ATOM | 77 | CG | LEU | A | 16 | 112.286 | −72.277 | 90.669 | 1.00 | 75.83 | | C |
| ANISOU | 77 | CG | LEU | A | 16 | 8413 | 8878 | 11519 | 1551 | 2266 | 854 | C |
| ATOM | 78 | CD1 | LEU | A | 16 | 113.575 | −72.167 | 89.948 | 1.00 | 75.11 | | C |
| ANISOU | 78 | CD1 | LEU | A | 16 | 8146 | 8920 | 11473 | 1599 | 2192 | 839 | C |
| ATOM | 79 | CD2 | LEU | A | 16 | 111.150 | −71.776 | 89.816 | 1.00 | 77.35 | | C |
| ANISOU | 79 | CD2 | LEU | A | 16 | 8668 | 8890 | 11830 | 1169 | 2251 | 630 | C |
| ATOM | 80 | N | ALA | A | 17 | 111.128 | −75.827 | 93.890 | 1.00 | 77.86 | | N |
| ANISOU | 80 | N | ALA | A | 17 | 9440 | 8611 | 11533 | 2635 | 3396 | 1767 | N |
| ATOM | 81 | CA | ALA | A | 17 | 111.077 | −77.219 | 94.364 | 1.00 | 77.52 | | C |
| ANISOU | 81 | CA | ALA | A | 17 | 9711 | 8224 | 11518 | 3011 | 3975 | 2104 | C |
| ATOM | 82 | C | ALA | A | 17 | 109.812 | −77.992 | 93.930 | 1.00 | 79.71 | | C |
| ANISOU | 82 | C | ALA | A | 17 | 10300 | 7900 | 12085 | 2660 | 4470 | 1948 | C |
| ATOM | 83 | O | ALA | A | 17 | 109.896 | −79.205 | 93.661 | 1.00 | 78.76 | | O |
| ANISOU | 83 | O | ALA | A | 17 | 10445 | 7333 | 12146 | 2786 | 5058 | 2089 | O |
| ATOM | 84 | CB | ALA | A | 17 | 111.222 | −77.256 | 95.871 | 1.00 | 78.37 | | C |
| ANISOU | 84 | CB | ALA | A | 17 | 9829 | 8651 | 11298 | 3526 | 3957 | 2437 | C |
| ATOM | 85 | N | TYR | A | 18 | 108.660 | −77.270 | 93.830 | 1.00 | 75.00 | | N |
| ANISOU | 85 | N | TYR | A | 18 | 9650 | 7314 | 11534 | 2210 | 4260 | 1625 | N |
| ATOM | 86 | CA | TYR | A | 18 | 107.339 | −77.781 | 93.424 | 1.00 | 74.75 | | C |
| ANISOU | 86 | CA | TYR | A | 18 | 9799 | 6871 | 11730 | 1793 | 4637 | 1354 | C |
| ATOM | 87 | C | TYR | A | 18 | 107.218 | −78.247 | 91.943 | 1.00 | 78.31 | | C |
| ANISOU | 87 | C | TYR | A | 18 | 10238 | 7072 | 12443 | 1388 | 4860 | 1021 | C |
| ATOM | 88 | O | TYR | A | 18 | 106.190 | −78.837 | 91.558 | 1.00 | 77.33 | | O |
| ANISOU | 88 | O | TYR | A | 18 | 10230 | 6644 | 12507 | 1026 | 5254 | 732 | O |
| ATOM | 89 | CB | TYR | A | 18 | 106.261 | −76.735 | 93.734 | 1.00 | 76.15 | | C |
| ANISOU | 89 | CB | TYR | A | 18 | 9844 | 7277 | 11813 | 1494 | 4268 | 1114 | C |
| ATOM | 90 | CG | TYR | A | 18 | 106.282 | −75.521 | 92.831 | 1.00 | 78.24 | | C |
| ANISOU | 90 | CG | TYR | A | 18 | 9813 | 7866 | 12047 | 1186 | 3746 | 828 | C |
| ATOM | 91 | CD2 | TYR | A | 18 | 105.425 | −75.421 | 91.742 | 1.00 | 79.22 | | C |
| ANISOU | 91 | CD2 | TYR | A | 18 | 9861 | 7939 | 12300 | 744 | 3780 | 455 | C |
| ATOM | 92 | CD1 | TYR | A | 18 | 107.111 | −74.436 | 93.106 | 1.00 | 79.71 | | C |
| ANISOU | 92 | CD1 | TYR | A | 18 | 9785 | 8444 | 12056 | 1346 | 3256 | 915 | C |
| ATOM | 93 | CE2 | TYR | A | 18 | 105.436 | −74.306 | 90.911 | 1.00 | 79.77 | | C |
| ANISOU | 93 | CE2 | TYR | A | 18 | 9687 | 8312 | 12310 | 552 | 3358 | 262 | C |
| ATOM | 94 | CE1 | TYR | A | 18 | 107.107 | −73.301 | 92.301 | 1.00 | 78.85 | | C |
| ANISOU | 94 | CE1 | TYR | A | 18 | 9457 | 8556 | 11945 | 1087 | 2879 | 684 | C |
| ATOM | 95 | CZ | TYR | A | 18 | 106.271 | −73.245 | 91.201 | 1.00 | 84.13 | | C |
| ANISOU | 95 | CZ | TYR | A | 18 | 10094 | 9143 | 12729 | 730 | 2938 | 401 | C |
| ATOM | 96 | OH | TYR | A | 18 | 106.230 | −72.136 | 90.404 | 1.00 | 84.88 | | O |
| ANISOU | 96 | OH | TYR | A | 18 | 10001 | 9461 | 12790 | 558 | 2624 | 238 | O |
| ATOM | 97 | N | LEU | A | 19 | 108.244 | −77.929 | 91.113 | 1.00 | 73.65 | | N |
| ANISOU | 97 | N | LEU | A | 19 | 9470 | 6666 | 11849 | 1421 | 4600 | 1011 | N |
| ATOM | 98 | CA | LEU | A | 19 | 108.289 | −78.265 | 89.694 | 1.00 | 72.59 | | C |
| ANISOU | 98 | CA | LEU | A | 19 | 9283 | 6389 | 11908 | 1085 | 4745 | 711 | C |
| ATOM | 99 | C | LEU | A | 19 | 109.090 | −79.541 | 89.445 | 1.00 | 78.33 | | C |
| ANISOU | 99 | C | LEU | A | 19 | 10223 | 6733 | 12805 | 1315 | 5289 | 899 | C |
| ATOM | 100 | O | LEU | A | 19 | 110.053 | −79.830 | 90.160 | 1.00 | 78.14 | | O |
| ANISOU | 100 | O | LEU | A | 19 | 10276 | 6734 | 12677 | 1827 | 5345 | 1318 | O |
| ATOM | 101 | CB | LEU | A | 19 | 108.902 | −77.117 | 88.874 | 1.00 | 71.94 | | C |
| ANISOU | 101 | CB | LEU | A | 19 | 8897 | 6708 | 11729 | 981 | 4179 | 593 | C |
| ATOM | 102 | CG | LEU | A | 19 | 108.189 | −75.776 | 88.823 | 1.00 | 75.11 | | C |
| ANISOU | 102 | CG | LEU | A | 19 | 9092 | 7454 | 11993 | 750 | 3698 | 397 | C |
| ATOM | 103 | CD1 | LEU | A | 19 | 109.142 | −74.721 | 88.362 | 1.00 | 74.57 | | C |
| ANISOU | 103 | CD1 | LEU | A | 19 | 8798 | 7696 | 11837 | 809 | 3257 | 428 | C |
| ATOM | 104 | CD2 | LEU | A | 19 | 106.957 | −75.815 | 87.916 | 1.00 | 75.95 | | C |
| ANISOU | 104 | CD2 | LEU | A | 19 | 9142 | 7551 | 12166 | 311 | 3806 | −7 | C |
| ATOM | 105 | N | ALA | A | 20 | 108.707 | −80.277 | 88.392 | 1.00 | 75.69 | | N |
| ANISOU | 105 | N | ALA | A | 20 | 9955 | 6097 | 12707 | 946 | 5695 | 565 | N |
| ATOM | 106 | CA | ALA | A | 20 | 109.371 | −81.497 | 87.967 | 1.00 | 76.34 | | C |
| ANISOU | 106 | CA | ALA | A | 20 | 10252 | 5750 | 13002 | 1078 | 6288 | 662 | C |
| ATOM | 107 | C | ALA | A | 20 | 110.773 | −81.140 | 87.499 | 1.00 | 83.88 | | C |
| ANISOU | 107 | C | ALA | A | 20 | 11041 | 6963 | 13868 | 1360 | 5944 | 871 | C |
| ATOM | 108 | O | ALA | A | 20 | 110.960 | −80.033 | 86.993 | 1.00 | 83.89 | | O |
| ANISOU | 108 | O | ALA | A | 20 | 10746 | 7393 | 13735 | 1205 | 5346 | 724 | O |
| ATOM | 109 | CB | ALA | A | 20 | 108.597 | −82.126 | 86.823 | 1.00 | 77.22 | | C |
| ANISOU | 109 | CB | ALA | A | 20 | 10374 | 5608 | 13360 | 507 | 6703 | 119 | C |
| ATOM | 110 | N | PRO | A | 21 | 111.776 | −82.038 | 87.631 | 1.00 | 82.79 | | N |
| ANISOU | 110 | N | PRO | A | 21 | 11084 | 6573 | 13802 | 1786 | 6342 | 1214 | N |
| ATOM | 111 | CA | PRO | A | 21 | 113.140 | −81.690 | 87.194 | 1.00 | 82.87 | | C |
| ANISOU | 111 | CA | PRO | A | 21 | 10893 | 6880 | 13713 | 2055 | 6007 | 1391 | C |
| ATOM | 112 | C | PRO | A | 21 | 113.253 | −81.067 | 85.799 | 1.00 | 87.24 | | C |
| ANISOU | 112 | C | PRO | A | 21 | 11191 | 7632 | 14324 | 1611 | 5665 | 994 | C |
| ATOM | 113 | O | PRO | A | 21 | 114.002 | −80.098 | 85.641 | 1.00 | 87.30 | | O |
| ANISOU | 113 | O | PRO | A | 21 | 10930 | 8068 | 14172 | 1698 | 5118 | 1048 | O |
| ATOM | 114 | CB | PRO | A | 21 | 113.883 | −83.020 | 87.299 | 1.00 | 84.90 | | C |
| ANISOU | 114 | CB | PRO | A | 21 | 11439 | 6708 | 14110 | 2483 | 6686 | 1727 | C |
| ATOM | 115 | CG | PRO | A | 21 | 113.205 | −83.717 | 88.416 | 1.00 | 89.63 | | C |
| ANISOU | 115 | CG | PRO | A | 21 | 12363 | 6965 | 14726 | 2722 | 7192 | 1971 | C |
| ATOM | 116 | CD | PRO | A | 21 | 111.748 | −83.385 | 88.240 | 1.00 | 85.22 | | C |
| ANISOU | 116 | CD | PRO | A | 21 | 11792 | 6320 | 14269 | 2099 | 7151 | 1497 | C |
| ATOM | 117 | N | GLU | A | 22 | 112.474 | −81.581 | 84.812 | 1.00 | 83.13 | | N |
| ANISOU | 117 | N | GLU | A | 22 | 10733 | 6841 | 14011 | 1126 | 6004 | 568 | N |
| ATOM | 118 | CA | GLU | A | 22 | 112.448 | −81.101 | 83.420 | 1.00 | 82.34 | | C |
| ANISOU | 118 | CA | GLU | A | 22 | 10395 | 6962 | 13930 | 720 | 5751 | 173 | C |
| ATOM | 119 | C | GLU | A | 22 | 111.928 | −79.667 | 83.354 | 1.00 | 80.83 | | C |
| ANISOU | 119 | C | GLU | A | 22 | 9926 | 7265 | 13519 | 533 | 5092 | 28 | C |
| ATOM | 120 | O | GLU | A | 22 | 112.372 | −78.873 | 82.520 | 1.00 | 79.73 | | O |
| ANISOU | 120 | O | GLU | A | 22 | 9564 | 7432 | 13297 | 448 | 4712 | −63 | O |
| ATOM | 121 | CB | GLU | A | 22 | 111.559 | −82.027 | 82.573 | 1.00 | 84.38 | | C |
| ANISOU | 121 | CB | GLU | A | 22 | 10750 | 6906 | 14404 | 246 | 6305 | −305 | C |
| ATOM | 122 | CG | GLU | A | 22 | 111.669 | −81.797 | 81.072 | 1.00 | 99.65 | | C |
| ANISOU | 122 | CG | GLU | A | 22 | 12450 | 9076 | 16334 | −100 | 6159 | −696 | C |
| ATOM | 123 | CD | GLU | A | 22 | 110.916 | −82.782 | 80.192 | 1.00 | 137.42 | | C |
| ANISOU | 123 | CD | GLU | A | 22 | 17275 | 13629 | 21310 | −583 | 6737 | −1243 | C |
| ATOM | 124 | OE1 | GLU | A | 22 | 109.997 | −83.475 | 80.696 | 1.00 | 140.60 | | O |
| ANISOU | 124 | OE1 | GLU | A | 22 | 17844 | 13726 | 21850 | −783 | 7217 | −1434 | O |
| ATOM | 125 | OE2 | GLU | A | 22 | 111.252 | −82.855 | 78.987 | 1.00 | 132.73 | | O |
| ANISOU | 125 | OE2 | GLU | A | 22 | 16528 | 13176 | 20725 | −782 | 6733 | −1513 | O |
| ATOM | 126 | N | GLU | A | 23 | 110.971 | −79.357 | 84.233 | 1.00 | 73.69 | | N |
| ANISOU | 126 | N | GLU | A | 23 | 9063 | 6404 | 12533 | 487 | 5016 | 23 | N |
| ATOM | 127 | CA | GLU | A | 23 | 110.374 | −78.040 | 84.335 | 1.00 | 71.87 | | C |
| ANISOU | 127 | CA | GLU | A | 23 | 8618 | 6583 | 12105 | 355 | 4475 | −77 | C |
| ATOM | 128 | C | GLU | A | 23 | 111.385 | −77.090 | 84.920 | 1.00 | 73.33 | | C |
| ANISOU | 128 | C | GLU | A | 23 | 8684 | 7035 | 12142 | 688 | 4018 | 244 | C |
| ATOM | 129 | O | GLU | A | 23 | 111.517 | −75.989 | 84.404 | 1.00 | 73.31 | | O |
| ANISOU | 129 | O | GLU | A | 23 | 8476 | 7338 | 12041 | 586 | 3615 | 156 | O |
| ATOM | 130 | CB | GLU | A | 23 | 109.099 | −78.068 | 85.177 | 1.00 | 72.92 | | C |
| ANISOU | 130 | CB | GLU | A | 23 | 8840 | 6660 | 12206 | 231 | 4573 | −167 | C |
| ATOM | 131 | CG | GLU | A | 23 | 107.938 | −78.745 | 84.476 | 1.00 | 80.27 | | C |
| ANISOU | 131 | CG | GLU | A | 23 | 9775 | 7474 | 13248 | −215 | 4955 | −633 | C |
| ATOM | 132 | CD | GLU | A | 23 | 106.651 | −78.693 | 85.261 | 1.00 | 92.90 | | C |
| ANISOU | 132 | CD | GLU | A | 23 | 11419 | 9069 | 14810 | −369 | 5035 | −763 | C |
| ATOM | 133 | OE1 | GLU | A | 23 | 106.567 | −79.375 | 86.308 | 1.00 | 95.82 | | O |
| ANISOU | 133 | OE1 | GLU | A | 23 | 12047 | 9084 | 15275 | −192 | 5390 | −546 | O |
| ATOM | 134 | OE2 | GLU | A | 23 | 105.727 | −77.967 | 84.830 | 1.00 | 82.10 | | O |
| ANISOU | 134 | OE2 | GLU | A | 23 | 9824 | 8072 | 13297 | −630 | 4762 | −1060 | O |
| ATOM | 135 | N | ARG | A | 24 | 112.135 | −77.515 | 85.954 | 1.00 | 67.69 | | N |
| ANISOU | 135 | N | ARG | A | 24 | 8081 | 6238 | 11400 | 1098 | 4120 | 602 | N |
| ATOM | 136 | CA | ARG | A | 24 | 113.152 | −76.666 | 86.568 | 1.00 | 66.88 | | C |
| ANISOU | 136 | CA | ARG | A | 24 | 7799 | 6487 | 11127 | 1399 | 3707 | 832 | C |
| ATOM | 137 | C | ARG | A | 24 | 114.261 | −76.353 | 85.574 | 1.00 | 70.56 | | C |
| ANISOU | 137 | C | ARG | A | 24 | 8084 | 7094 | 11629 | 1389 | 3547 | 792 | C |
| ATOM | 138 | O | ARG | A | 24 | 114.716 | −75.214 | 85.504 | 1.00 | 70.51 | | O |
| ANISOU | 138 | O | ARG | A | 24 | 7858 | 7404 | 11529 | 1350 | 3147 | 745 | O |
| ATOM | 139 | CB | ARG | A | 24 | 113.721 | −77.282 | 87.850 | 1.00 | 67.71 | | C |
| ANISOU | 139 | CB | ARG | A | 24 | 8011 | 6593 | 11124 | 1899 | 3870 | 1213 | C |
| ATOM | 140 | CG | ARG | A | 24 | 112.782 | −77.207 | 89.057 | 1.00 | 85.68 | | C |
| ANISOU | 140 | CG | ARG | A | 24 | 10405 | 8864 | 13287 | 1969 | 3890 | 1297 | C |
| ATOM | 141 | CD | ARG | A | 24 | 113.497 | −77.510 | 90.362 | 1.00 | 104.46 | | C |
| ANISOU | 141 | CD | ARG | A | 24 | 12800 | 11441 | 15450 | 2539 | 3926 | 1696 | C |
| ATOM | 142 | NE | ARG | A | 24 | 114.198 | −78.794 | 90.318 | 1.00 | 123.75 | | N |
| ANISOU | 142 | NE | ARG | A | 24 | 15434 | 13622 | 17962 | 2932 | 4423 | 1995 | N |
| ATOM | 143 | CZ | ARG | A | 24 | 113.705 | −79.937 | 90.783 | 1.00 | 138.77 | | C |
| ANISOU | 143 | CZ | ARG | A | 24 | 17682 | 15088 | 19955 | 3135 | 5016 | 2208 | C |
| ATOM | 144 | NH1 | ARG | A | 24 | 112.514 | −79.965 | 91.365 | 1.00 | 123.87 | | N |
| ANISOU | 144 | NH1 | ARG | A | 24 | 15964 | 13012 | 18090 | 2965 | 5154 | 2137 | N |
| ATOM | 145 | NH2 | ARG | A | 24 | 114.412 | −81.057 | 90.694 | 1.00 | 125.12 | | N |
| ANISOU | 145 | NH2 | ARG | A | 24 | 16146 | 13092 | 18301 | 3530 | 5521 | 2506 | N |
| ATOM | 146 | N | ALA | A | 25 | 114.668 | −77.338 | 84.769 | 1.00 | 66.28 | | N |
| ANISOU | 146 | N | ALA | A | 25 | 7645 | 6292 | 11246 | 1393 | 3902 | 781 | N |
| ATOM | 147 | CA | ALA | A | 25 | 115.696 | −77.119 | 83.755 | 1.00 | 65.24 | | C |
| ANISOU | 147 | CA | ALA | A | 25 | 7355 | 6273 | 11161 | 1374 | 3789 | 734 | C |
| ATOM | 148 | C | ALA | A | 25 | 115.238 | −76.044 | 82.766 | 1.00 | 68.29 | | C |
| ANISOU | 148 | C | ALA | A | 25 | 7576 | 6847 | 11525 | 999 | 3483 | 444 | C |
| ATOM | 149 | O | ALA | A | 25 | 116.056 | −75.211 | 82.384 | 1.00 | 68.58 | | O |
| ANISOU | 149 | O | ALA | A | 25 | 7425 | 7110 | 11523 | 1016 | 3209 | 451 | O |
| ATOM | 150 | CB | ALA | A | 25 | 116.006 | −78.412 | 83.026 | 1.00 | 66.05 | | C |
| ANISOU | 150 | CB | ALA | A | 25 | 7626 | 6019 | 11452 | 1397 | 4281 | 729 | C |
| ATOM | 151 | N | LYS | A | 26 | 113.920 | −76.018 | 82.408 | 1.00 | 63.62 | | N |
| ANISOU | 151 | N | LYS | A | 26 | 7040 | 6197 | 10934 | 690 | 3551 | 195 | N |
| ATOM | 152 | CA | LYS | A | 26 | 113.334 | −75.009 | 81.508 | 1.00 | 62.33 | | C |
| ANISOU | 152 | CA | LYS | A | 26 | 6722 | 6276 | 10684 | 424 | 3296 | −34 | C |
| ATOM | 153 | C | LYS | A | 26 | 113.497 | −73.628 | 82.119 | 1.00 | 65.50 | | C |
| ANISOU | 153 | C | LYS | A | 26 | 7002 | 6921 | 10964 | 507 | 2900 | 79 | C |
| ATOM | 154 | O | LYS | A | 26 | 113.975 | −72.719 | 81.435 | 1.00 | 64.92 | | O |
| ANISOU | 154 | O | LYS | A | 26 | 6796 | 7007 | 10863 | 468 | 2712 | 54 | O |
| ATOM | 155 | CB | LYS | A | 26 | 111.848 | −75.292 | 81.201 | 1.00 | 63.03 | | C |
| ANISOU | 155 | CB | LYS | A | 26 | 6843 | 6368 | 10738 | 138 | 3446 | −323 | C |
| ATOM | 156 | CG | LYS | A | 26 | 111.260 | −74.377 | 80.129 | 1.00 | 64.72 | | C |
| ANISOU | 156 | CG | LYS | A | 26 | 6872 | 6919 | 10800 | −46 | 3231 | −534 | C |
| ATOM | 157 | CD | LYS | A | 26 | 109.858 | −74.788 | 79.739 | 1.00 | 79.18 | | C |
| ANISOU | 157 | CD | LYS | A | 26 | 8657 | 8875 | 12553 | −317 | 3396 | −878 | C |
| ATOM | 158 | CE | LYS | A | 26 | 109.493 | −74.255 | 78.381 | 1.00 | 97.27 | | C |
| ANISOU | 158 | CE | LYS | A | 26 | 10734 | 11567 | 14658 | −435 | 3283 | −1100 | C |
| ATOM | 159 | NZ | LYS | A | 26 | 108.217 | −74.824 | 77.870 | 1.00 | 112.93 | | N |
| ANISOU | 159 | NZ | LYS | A | 26 | 12589 | 13788 | 16529 | −719 | 3475 | −1530 | N |
| ATOM | 160 | N | VAL | A | 27 | 113.137 | −73.488 | 83.418 | 1.00 | 61.44 | | N |
| ANISOU | 160 | N | VAL | A | 27 | 6544 | 6412 | 10387 | 624 | 2825 | 197 | N |
| ATOM | 161 | CA | VAL | A | 27 | 113.244 | −72.240 | 84.185 | 1.00 | 60.14 | | C |
| ANISOU | 161 | CA | VAL | A | 27 | 6272 | 6461 | 10117 | 679 | 2503 | 259 | C |
| ATOM | 162 | C | VAL | A | 27 | 114.721 | −71.761 | 84.251 | 1.00 | 63.94 | | C |
| ANISOU | 162 | C | VAL | A | 27 | 6589 | 7094 | 10609 | 827 | 2355 | 350 | C |
| ATOM | 163 | O | VAL | A | 27 | 114.957 | −70.571 | 84.036 | 1.00 | 62.76 | | O |
| ANISOU | 163 | O | VAL | A | 27 | 6315 | 7085 | 10445 | 727 | 2167 | 273 | O |
| ATOM | 164 | CB | VAL | A | 27 | 112.551 | −72.355 | 85.565 | 1.00 | 62.94 | | C |
| ANISOU | 164 | CB | VAL | A | 27 | 6719 | 6806 | 10389 | 777 | 2492 | 345 | C |
| ATOM | 165 | CG1 | VAL | A | 27 | 112.863 | −71.164 | 86.447 | 1.00 | 62.84 | | C |
| ANISOU | 165 | CG1 | VAL | A | 27 | 6575 | 7032 | 10270 | 842 | 2194 | 379 | C |
| ATOM | 166 | CG2 | VAL | A | 27 | 111.046 | −72.504 | 85.406 | 1.00 | 62.40 | | C |
| ANISOU | 166 | CG2 | VAL | A | 27 | 6750 | 6655 | 10306 | 565 | 2596 | 186 | C |
| ATOM | 167 | N | ARG | A | 28 | 115.708 | −72.693 | 84.471 | 1.00 | 60.56 | | N |
| ANISOU | 167 | N | ARG | A | 28 | 6159 | 6638 | 10212 | 1065 | 2492 | 499 | N |
| ATOM | 168 | CA | ARG | A | 28 | 117.144 | −72.356 | 84.488 | 1.00 | 59.76 | | C |
| ANISOU | 168 | CA | ARG | A | 28 | 5848 | 6759 | 10101 | 1212 | 2368 | 546 | C |
| ATOM | 169 | C | ARG | A | 28 | 117.588 | −71.895 | 83.115 | 1.00 | 60.38 | | C |
| ANISOU | 169 | C | ARG | A | 28 | 5849 | 6802 | 10290 | 1015 | 2361 | 415 | C |
| ATOM | 170 | O | ARG | A | 28 | 118.279 | −70.884 | 83.016 | 1.00 | 60.45 | | O |
| ANISOU | 170 | O | ARG | A | 28 | 5677 | 6988 | 10304 | 945 | 2201 | 332 | O |
| ATOM | 171 | CB | ARG | A | 28 | 118.022 | −73.520 | 84.985 | 1.00 | 62.04 | | C |
| ANISOU | 171 | CB | ARG | A | 28 | 6145 | 7070 | 10358 | 1584 | 2546 | 772 | C |
| ATOM | 172 | CG | ARG | A | 28 | 119.496 | −73.141 | 85.169 | 1.00 | 75.22 | | C |
| ANISOU | 172 | CG | ARG | A | 28 | 7519 | 9106 | 11954 | 1768 | 2383 | 792 | C |
| ATOM | 173 | CD | ARG | A | 28 | 120.245 | −74.112 | 86.057 | 1.00 | 91.76 | | C |
| ANISOU | 173 | CD | ARG | A | 28 | 9574 | 11388 | 13902 | 2258 | 2505 | 1065 | C |
| ATOM | 174 | NE | ARG | A | 28 | 121.041 | −75.062 | 85.277 | 1.00 | 108.66 | | N |
| ANISOU | 174 | NE | ARG | A | 28 | 11757 | 13383 | 16147 | 2436 | 2758 | 1193 | N |
| ATOM | 175 | CZ | ARG | A | 28 | 120.643 | −76.285 | 84.937 | 1.00 | 131.19 | | C |
| ANISOU | 175 | CZ | ARG | A | 28 | 14911 | 15812 | 19122 | 2544 | 3157 | 1350 | C |
| ATOM | 176 | NH1 | ARG | A | 28 | 119.445 | −76.730 | 85.303 | 1.00 | 121.07 | | N |
| ANISOU | 176 | NH1 | ARG | A | 28 | 13904 | 14219 | 17878 | 2472 | 3355 | 1381 | N |
| ATOM | 177 | NH2 | ARG | A | 28 | 121.439 | −77.074 | 84.228 | 1.00 | 122.81 | | N |
| ANISOU | 177 | NH2 | ARG | A | 28 | 13877 | 14622 | 18165 | 2701 | 3403 | 1448 | N |
| ATOM | 178 | N | GLU | A | 29 | 117.160 | −72.608 | 82.058 | 1.00 | 54.21 | | N |
| ANISOU | 178 | N | GLU | A | 29 | 5201 | 5802 | 9595 | 905 | 2571 | 365 | N |
| ATOM | 179 | CA | GLU | A | 29 | 117.468 | −72.249 | 80.677 | 1.00 | 53.07 | | C |
| ANISOU | 179 | CA | GLU | A | 29 | 4997 | 5649 | 9517 | 745 | 2589 | 252 | C |
| ATOM | 180 | C | GLU | A | 29 | 116.900 | −70.895 | 80.332 | 1.00 | 54.80 | | C |
| ANISOU | 180 | C | GLU | A | 29 | 5168 | 5973 | 9681 | 572 | 2424 | 154 | C |
| ATOM | 181 | O | GLU | A | 29 | 117.542 | −70.169 | 79.580 | 1.00 | 56.56 | | O |
| ANISOU | 181 | O | GLU | A | 29 | 5299 | 6247 | 9944 | 518 | 2398 | 124 | O |
| ATOM | 182 | CB | GLU | A | 29 | 116.992 | −73.317 | 79.706 | 1.00 | 54.45 | | C |
| ANISOU | 182 | CB | GLU | A | 29 | 5297 | 5639 | 9751 | 649 | 2857 | 162 | C |
| ATOM | 183 | CG | GLU | A | 29 | 118.123 | −74.222 | 79.250 | 1.00 | 67.87 | | C |
| ANISOU | 183 | CG | GLU | A | 29 | 6991 | 7234 | 11563 | 785 | 3051 | 234 | C |
| ATOM | 184 | CD | GLU | A | 29 | 119.008 | −74.822 | 80.331 | 1.00 | 90.75 | | C |
| ANISOU | 184 | CD | GLU | A | 29 | 9882 | 10133 | 14467 | 1115 | 3103 | 464 | C |
| ATOM | 185 | OE1 | GLU | A | 29 | 120.249 | −74.693 | 80.214 | 1.00 | 65.87 | | O |
| ANISOU | 185 | OE1 | GLU | A | 29 | 6573 | 7124 | 11332 | 1267 | 3041 | 540 | O |
| ATOM | 186 | OE2 | GLU | A | 29 | 118.470 | −75.423 | 81.290 | 1.00 | 89.25 | | O |
| ANISOU | 186 | OE2 | GLU | A | 29 | 9829 | 9839 | 14243 | 1251 | 3223 | 573 | O |
| ATOM | 187 | N | ALA | A | 30 | 115.747 | −70.530 | 80.936 | 1.00 | 47.27 | | N |
| ANISOU | 187 | N | ALA | A | 30 | 4286 | 5037 | 8638 | 515 | 2351 | 128 | N |
| ATOM | 188 | CA | ALA | A | 30 | 115.102 | −69.224 | 80.796 | 1.00 | 46.03 | | C |
| ANISOU | 188 | CA | ALA | A | 30 | 4112 | 4964 | 8413 | 418 | 2237 | 83 | C |
| ATOM | 189 | C | ALA | A | 30 | 115.907 | −68.139 | 81.526 | 1.00 | 46.97 | | C |
| ANISOU | 189 | C | ALA | A | 30 | 4122 | 5149 | 8574 | 430 | 2114 | 93 | C |
| ATOM | 190 | O | ALA | A | 30 | 116.165 | −67.087 | 80.942 | 1.00 | 45.37 | | O |
| ANISOU | 190 | O | ALA | A | 30 | 3884 | 4940 | 8414 | 358 | 2143 | 62 | O |
| ATOM | 191 | CB | ALA | A | 30 | 113.676 | −69.275 | 81.332 | 1.00 | 46.78 | | C |
| ANISOU | 191 | CB | ALA | A | 30 | 4301 | 5074 | 8400 | 376 | 2212 | 49 | C |
| ATOM | 192 | N | TYR | A | 31 | 116.308 | −68.400 | 82.797 | 1.00 | 42.07 | | N |
| ANISOU | 192 | N | TYR | A | 31 | 3439 | 4612 | 7931 | 527 | 2022 | 118 | N |
| ATOM | 193 | CA | TYR | A | 31 | 117.118 | −67.474 | 83.598 | 1.00 | 40.68 | | C |
| ANISOU | 193 | CA | TYR | A | 31 | 3090 | 4601 | 7767 | 505 | 1912 | 33 | C |
| ATOM | 194 | C | TYR | A | 31 | 118.475 | −67.243 | 82.918 | 1.00 | 45.68 | | C |
| ANISOU | 194 | C | TYR | A | 31 | 3554 | 5292 | 8509 | 470 | 1966 | −36 | C |
| ATOM | 195 | O | TYR | A | 31 | 118.962 | −66.117 | 82.903 | 1.00 | 45.09 | | O |
| ANISOU | 195 | O | TYR | A | 31 | 3368 | 5255 | 8511 | 319 | 1986 | −184 | O |
| ATOM | 196 | CB | TYR | A | 31 | 117.338 | −68.000 | 85.032 | 1.00 | 40.72 | | C |
| ANISOU | 196 | CB | TYR | A | 31 | 3010 | 4810 | 7650 | 681 | 1800 | 74 | C |
| ATOM | 197 | CG | TYR | A | 31 | 118.529 | −67.363 | 85.713 | 1.00 | 41.82 | | C |
| ANISOU | 197 | CG | TYR | A | 31 | 2861 | 5266 | 7761 | 682 | 1696 | −78 | C |
| ATOM | 198 | CD1 | TYR | A | 31 | 118.406 | −66.156 | 86.384 | 1.00 | 42.91 | | C |
| ANISOU | 198 | CD1 | TYR | A | 31 | 2899 | 5515 | 7890 | 506 | 1627 | −280 | C |
| ATOM | 199 | CD2 | TYR | A | 31 | 119.799 | −67.919 | 85.603 | 1.00 | 44.12 | | C |
| ANISOU | 199 | CD2 | TYR | A | 31 | 2953 | 5768 | 8043 | 831 | 1700 | −67 | C |
| ATOM | 200 | CE1 | TYR | A | 31 | 119.512 | −65.537 | 86.971 | 1.00 | 44.71 | | C |
| ANISOU | 200 | CE1 | TYR | A | 31 | 2807 | 6088 | 8094 | 434 | 1568 | −527 | C |
| ATOM | 201 | CE2 | TYR | A | 31 | 120.925 | −67.269 | 86.112 | 1.00 | 45.69 | | C |
| ANISOU | 201 | CE2 | TYR | A | 31 | 2813 | 6346 | 8203 | 786 | 1613 | −289 | C |
| ATOM | 202 | CZ | TYR | A | 31 | 120.777 | −66.085 | 86.815 | 1.00 | 52.66 | | C |
| ANISOU | 202 | CZ | TYR | A | 31 | 3571 | 7366 | 9071 | 565 | 1550 | −549 | C |
| ATOM | 203 | OH | TYR | A | 31 | 121.892 | −65.466 | 87.346 | 1.00 | 50.30 | | O |
| ANISOU | 203 | OH | TYR | A | 31 | 2887 | 7499 | 8727 | 466 | 1497 | −864 | O |
| ATOM | 204 | N | ARG | A | 32 | 119.107 | −68.319 | 82.420 | 1.00 | 43.15 | | N |
| ANISOU | 204 | N | ARG | A | 32 | 3217 | 4968 | 8211 | 601 | 2030 | 52 | N |
| ATOM | 205 | CA | ARG | A | 32 | 120.400 | −68.239 | 81.758 | 1.00 | 44.68 | | C |
| ANISOU | 205 | CA | ARG | A | 32 | 3242 | 5232 | 8502 | 588 | 2087 | −5 | C |
| ATOM | 206 | C | ARG | A | 32 | 120.322 | −67.402 | 80.476 | 1.00 | 48.64 | | C |
| ANISOU | 206 | C | ARG | A | 32 | 3812 | 5554 | 9117 | 402 | 2215 | −59 | C |
| ATOM | 207 | O | ARG | A | 32 | 121.265 | −66.649 | 80.153 | 1.00 | 48.89 | | O |
| ANISOU | 207 | O | ARG | A | 32 | 3697 | 5623 | 9255 | 292 | 2282 | −178 | O |
| ATOM | 208 | CB | ARG | A | 32 | 120.987 | −69.635 | 81.505 | 1.00 | 49.66 | | C |
| ANISOU | 208 | CB | ARG | A | 32 | 3875 | 5866 | 9129 | 807 | 2168 | 133 | C |
| ATOM | 209 | CG | ARG | A | 32 | 121.741 | −70.198 | 82.713 | 1.00 | 67.45 | | C |
| ANISOU | 209 | CG | ARG | A | 32 | 5949 | 8421 | 11258 | 1082 | 2083 | 203 | C |
| ATOM | 210 | CD | ARG | A | 32 | 121.993 | −71.693 | 82.606 | 1.00 | 83.71 | | C |
| ANISOU | 210 | CD | ARG | A | 32 | 8113 | 10390 | 13301 | 1379 | 2252 | 424 | C |
| ATOM | 211 | NE | ARG | A | 32 | 122.777 | −72.066 | 81.424 | 1.00 | 93.81 | | N |
| ANISOU | 211 | NE | ARG | A | 32 | 9367 | 11567 | 14710 | 1361 | 2392 | 421 | N |
| ATOM | 212 | CZ | ARG | A | 32 | 123.253 | −73.285 | 81.195 | 1.00 | 113.80 | | C |
| ANISOU | 212 | CZ | ARG | A | 32 | 11963 | 14011 | 17263 | 1614 | 2590 | 590 | C |
| ATOM | 213 | NH1 | ARG | A | 32 | 123.043 | −74.263 | 82.075 | 1.00 | 100.56 | | N |
| ANISOU | 213 | NH1 | ARG | A | 32 | 10401 | 12316 | 15491 | 1935 | 2713 | 806 | N |
| ATOM | 214 | NH2 | ARG | A | 32 | 123.948 | −73.541 | 80.095 | 1.00 | 104.38 | | N |
| ANISOU | 214 | NH2 | ARG | A | 32 | 10741 | 12727 | 16191 | 1571 | 2714 | 561 | N |
| ATOM | 215 | N | PHE | A | 33 | 119.172 | −67.492 | 79.790 | 1.00 | 43.75 | | N |
| ANISOU | 215 | N | PHE | A | 33 | 3397 | 4777 | 8450 | 379 | 2274 | 15 | N |
| ATOM | 216 | CA | PHE | A | 33 | 118.905 | −66.713 | 78.603 | 1.00 | 43.70 | | C |
| ANISOU | 216 | CA | PHE | A | 33 | 3468 | 4668 | 8467 | 301 | 2407 | 24 | C |
| ATOM | 217 | C | PHE | A | 33 | 118.647 | −65.259 | 78.980 | 1.00 | 52.31 | | C |
| ANISOU | 217 | C | PHE | A | 33 | 4579 | 5703 | 9592 | 206 | 2453 | −22 | C |
| ATOM | 218 | O | PHE | A | 33 | 119.199 | −64.384 | 78.331 | 1.00 | 53.51 | | O |
| ANISOU | 218 | O | PHE | A | 33 | 4723 | 5758 | 9851 | 138 | 2632 | −45 | O |
| ATOM | 219 | CB | PHE | A | 33 | 117.739 | −67.285 | 77.797 | 1.00 | 44.95 | | C |
| ANISOU | 219 | CB | PHE | A | 33 | 3769 | 4818 | 8493 | 342 | 2447 | 81 | C |
| ATOM | 220 | CG | PHE | A | 33 | 117.515 | −66.503 | 76.521 | 1.00 | 46.45 | | C |
| ANISOU | 220 | CG | PHE | A | 33 | 4009 | 5009 | 8633 | 354 | 2589 | 129 | C |
| ATOM | 221 | CD1 | PHE | A | 33 | 118.328 | −66.712 | 75.408 | 1.00 | 48.31 | | C |
| ANISOU | 221 | CD1 | PHE | A | 33 | 4204 | 5236 | 8917 | 367 | 2713 | 139 | C |
| ATOM | 222 | CD2 | PHE | A | 33 | 116.543 | −65.512 | 76.451 | 1.00 | 49.12 | | C |
| ANISOU | 222 | CD2 | PHE | A | 33 | 4435 | 5369 | 8858 | 401 | 2627 | 196 | C |
| ATOM | 223 | CE1 | PHE | A | 33 | 118.143 | −65.973 | 74.241 | 1.00 | 48.72 | | C |
| ANISOU | 223 | CE1 | PHE | A | 33 | 4309 | 5317 | 8885 | 437 | 2873 | 224 | C |
| ATOM | 224 | CE2 | PHE | A | 33 | 116.369 | −64.761 | 75.281 | 1.00 | 51.48 | | C |
| ANISOU | 224 | CE2 | PHE | A | 33 | 4790 | 5701 | 9070 | 509 | 2806 | 306 | C |
| ATOM | 225 | CZ | PHE | A | 33 | 117.155 | −65.020 | 74.177 | 1.00 | 48.88 | | C |
| ANISOU | 225 | CZ | PHE | A | 33 | 4423 | 5381 | 8766 | 533 | 2927 | 323 | C |
| ATOM | 226 | N | ALA | A | 34 | 117.798 | −64.999 | 80.004 | 1.00 | 51.44 | | N |
| ANISOU | 226 | N | ALA | A | 34 | 4518 | 5621 | 9408 | 201 | 2345 | −35 | N |
| ATOM | 227 | CA | ALA | A | 34 | 117.486 | −63.652 | 80.497 | 1.00 | 51.92 | | C |
| ANISOU | 227 | CA | ALA | A | 34 | 4616 | 5597 | 9513 | 108 | 2426 | −96 | C |
| ATOM | 228 | C | ALA | A | 34 | 118.765 | −62.985 | 80.946 | 1.00 | 58.16 | | C |
| ANISOU | 228 | C | ALA | A | 34 | 5219 | 6409 | 10469 | −55 | 2503 | −302 | C |
| ATOM | 229 | O | ALA | A | 34 | 119.077 | −61.932 | 80.422 | 1.00 | 56.99 | | O |
| ANISOU | 229 | O | ALA | A | 34 | 5112 | 6083 | 10460 | −164 | 2761 | −353 | O |
| ATOM | 230 | CB | ALA | A | 34 | 116.501 | −63.719 | 81.648 | 1.00 | 52.34 | | C |
| ANISOU | 230 | CB | ALA | A | 34 | 4717 | 5715 | 9454 | 131 | 2268 | −100 | C |
| ATOM | 231 | N | GLU | A | 35 | 119.563 | −63.647 | 81.811 | 1.00 | 57.95 | | N |
| ANISOU | 231 | N | GLU | A | 35 | 4974 | 6628 | 10417 | −51 | 2325 | −422 | N |
| ATOM | 232 | CA | GLU | A | 35 | 120.848 | −63.121 | 82.280 | 1.00 | 59.40 | | C |
| ANISOU | 232 | CA | GLU | A | 35 | 4879 | 6984 | 10707 | −212 | 2369 | −696 | C |
| ATOM | 233 | C | GLU | A | 35 | 121.806 | −62.766 | 81.134 | 1.00 | 64.84 | | C |
| ANISOU | 233 | C | GLU | A | 35 | 5519 | 7543 | 11573 | −318 | 2603 | −750 | C |
| ATOM | 234 | O | GLU | A | 35 | 122.481 | −61.738 | 81.217 | 1.00 | 65.80 | | O |
| ANISOU | 234 | O | GLU | A | 35 | 5517 | 7625 | 11858 | −556 | 2814 | −1007 | O |
| ATOM | 235 | CB | GLU | A | 35 | 121.513 | −64.045 | 83.300 | 1.00 | 61.37 | | C |
| ANISOU | 235 | CB | GLU | A | 35 | 4868 | 7640 | 10808 | −75 | 2123 | −760 | C |
| ATOM | 236 | CG | GLU | A | 35 | 122.342 | −63.290 | 84.333 | 1.00 | 76.65 | | C |
| ANISOU | 236 | CG | GLU | A | 35 | 6467 | 9915 | 12740 | −253 | 2098 | −1126 | C |
| ATOM | 237 | CD | GLU | A | 35 | 123.626 | −62.649 | 83.830 | 1.00 | 100.46 | | C |
| ANISOU | 237 | CD | GLU | A | 35 | 9232 | 13004 | 15933 | −487 | 2290 | −1419 | C |
| ATOM | 238 | OE1 | GLU | A | 35 | 124.490 | −63.384 | 83.296 | 1.00 | 83.24 | | O |
| ANISOU | 238 | OE1 | GLU | A | 35 | 6915 | 10966 | 13746 | −363 | 2258 | −1358 | O |
| ATOM | 239 | OE2 | GLU | A | 35 | 123.752 | −61.407 | 83.942 | 1.00 | 95.30 | | O |
| ANISOU | 239 | OE2 | GLU | A | 35 | 8533 | 12231 | 15445 | −805 | 2521 | −1718 | O |
| ATOM | 240 | N | GLU | A | 36 | 121.826 | −63.573 | 80.052 | 1.00 | 59.59 | | N |
| ANISOU | 240 | N | GLU | A | 36 | 4959 | 6794 | 10887 | −169 | 2614 | −538 | N |
| ATOM | 241 | CA | GLU | A | 36 | 122.603 | −63.286 | 78.854 | 1.00 | 58.22 | | C |
| ANISOU | 241 | CA | GLU | A | 36 | 4782 | 6480 | 10860 | −234 | 2848 | −541 | C |
| ATOM | 242 | C | GLU | A | 36 | 122.006 | −62.086 | 78.096 | 1.00 | 59.39 | | C |
| ANISOU | 242 | C | GLU | A | 36 | 5169 | 6301 | 11096 | −296 | 3162 | −460 | C |
| ATOM | 243 | O | GLU | A | 36 | 122.775 | −61.246 | 77.641 | 1.00 | 60.52 | | O |
| ANISOU | 243 | O | GLU | A | 36 | 5277 | 6290 | 11428 | −453 | 3461 | −580 | O |
| ATOM | 244 | CB | GLU | A | 36 | 122.648 | −64.514 | 77.949 | 1.00 | 60.00 | | C |
| ANISOU | 244 | CB | GLU | A | 36 | 5066 | 6724 | 11008 | −45 | 2778 | −345 | C |
| ATOM | 245 | CG | GLU | A | 36 | 123.766 | −64.495 | 76.911 | 1.00 | 80.10 | | C |
| ANISOU | 245 | CG | GLU | A | 36 | 7523 | 9226 | 13686 | −92 | 2959 | −380 | C |
| ATOM | 246 | CD | GLU | A | 36 | 125.202 | −64.656 | 77.387 | 1.00 | 116.81 | | C |
| ANISOU | 246 | CD | GLU | A | 36 | 11840 | 14119 | 18423 | −168 | 2923 | −599 | C |
| ATOM | 247 | OE1 | GLU | A | 36 | 125.665 | −65.815 | 77.488 | 1.00 | 124.68 | | O |
| ANISOU | 247 | OE1 | GLU | A | 36 | 12725 | 15307 | 19340 | 8 | 2773 | −532 | O |
| ATOM | 248 | OE2 | GLU | A | 36 | 125.878 | −63.626 | 77.621 | 1.00 | 114.15 | | O |
| ANISOU | 248 | OE2 | GLU | A | 36 | 11345 | 13792 | 18234 | −400 | 3088 | −851 | O |
| ATOM | 249 | N | ALA | A | 37 | 120.651 | −61.997 | 77.982 | 1.00 | 52.50 | | N |
| ANISOU | 249 | N | ALA | A | 37 | 4530 | 5338 | 10080 | −153 | 3132 | −256 | N |
| ATOM | 250 | CA | ALA | A | 37 | 119.941 | −60.919 | 77.290 | 1.00 | 50.45 | | C |
| ANISOU | 250 | CA | ALA | A | 37 | 4509 | 4830 | 9831 | −88 | 3438 | −99 | C |
| ATOM | 251 | C | ALA | A | 37 | 120.008 | −59.588 | 78.022 | 1.00 | 58.05 | | C |
| ANISOU | 251 | C | ALA | A | 37 | 5505 | 5582 | 10970 | −275 | 3698 | −263 | C |
| ATOM | 252 | O | ALA | A | 37 | 120.203 | −58.562 | 77.373 | 1.00 | 59.71 | | O |
| ANISOU | 252 | O | ALA | A | 37 | 5860 | 5501 | 11327 | −298 | 4125 | −213 | O |
| ATOM | 253 | CB | ALA | A | 37 | 118.505 | −61.310 | 76.989 | 1.00 | 50.01 | | C |
| ANISOU | 253 | CB | ALA | A | 37 | 4618 | 4864 | 9520 | 149 | 3303 | 135 | C |
| ATOM | 254 | N | HIS | A | 38 | 119.909 | −59.595 | 79.366 | 1.00 | 56.10 | | N |
| ANISOU | 254 | N | HIS | A | 38 | 5128 | 5471 | 10718 | −411 | 3498 | −473 | N |
| ATOM | 255 | CA | HIS | A | 38 | 119.938 | −58.397 | 80.232 | 1.00 | 55.80 | | C |
| ANISOU | 255 | CA | HIS | A | 38 | 5086 | 5275 | 10840 | −641 | 3734 | −716 | C |
| ATOM | 256 | C | HIS | A | 38 | 121.311 | −58.098 | 80.787 | 1.00 | 62.85 | | C |
| ANISOU | 256 | C | HIS | A | 38 | 5668 | 6287 | 11924 | −971 | 3824 | −1146 | C |
| ATOM | 257 | O | HIS | A | 38 | 121.425 | −57.348 | 81.763 | 1.00 | 63.04 | | O |
| ANISOU | 257 | O | HIS | A | 38 | 5585 | 6324 | 12045 | −1219 | 3934 | −1469 | O |
| ATOM | 258 | CB | HIS | A | 38 | 118.927 | −58.506 | 81.370 | 1.00 | 55.63 | | C |
| ANISOU | 258 | CB | HIS | A | 38 | 5090 | 5383 | 10664 | −595 | 3479 | −721 | C |
| ATOM | 259 | CG | HIS | A | 38 | 117.528 | −58.541 | 80.877 | 1.00 | 58.94 | | C |
| ANISOU | 259 | CG | HIS | A | 38 | 5783 | 5702 | 10909 | −314 | 3465 | −369 | C |
| ATOM | 260 | ND1 | HIS | A | 38 | 117.015 | −57.517 | 80.119 | 1.00 | 61.06 | | N |
| ANISOU | 260 | ND1 | HIS | A | 38 | 6306 | 5665 | 11231 | −193 | 3865 | −172 | N |
| ATOM | 261 | CD2 | HIS | A | 38 | 116.591 | −59.500 | 81.005 | 1.00 | 61.15 | | C |
| ANISOU | 261 | CD2 | HIS | A | 38 | 6094 | 6188 | 10952 | −121 | 3133 | −198 | C |
| ATOM | 262 | CE1 | HIS | A | 38 | 115.776 | −57.865 | 79.833 | 1.00 | 60.62 | | C |
| ANISOU | 262 | CE1 | HIS | A | 38 | 6383 | 5722 | 10929 | 87 | 3715 | 101 | C |
| ATOM | 263 | NE2 | HIS | A | 38 | 115.472 | −59.045 | 80.359 | 1.00 | 61.10 | | N |
| ANISOU | 263 | NE2 | HIS | A | 38 | 6309 | 6078 | 10827 | 98 | 3281 | 59 | N |
| ATOM | 264 | N | ARG | A | 39 | 122.354 | −58.667 | 80.158 | 1.00 | 60.62 | | N |
| ANISOU | 264 | N | ARG | A | 39 | 5214 | 6131 | 11687 | −988 | 3794 | −1189 | N |
| ATOM | 265 | CA | ARG | A | 39 | 123.731 | −58.452 | 80.557 | 1.00 | 61.29 | | C |
| ANISOU | 265 | CA | ARG | A | 39 | 4941 | 6421 | 11924 | −1286 | 3877 | −1619 | C |
| ATOM | 266 | C | ARG | A | 39 | 124.139 | −57.014 | 80.221 | 1.00 | 66.45 | | C |
| ANISOU | 266 | C | ARG | A | 39 | 5679 | 6670 | 12899 | −1601 | 4480 | −1860 | C |
| ATOM | 267 | O | ARG | A | 39 | 123.954 | −56.553 | 79.086 | 1.00 | 65.46 | | O |
| ANISOU | 267 | O | ARG | A | 39 | 5851 | 6130 | 12892 | −1502 | 4852 | −1596 | O |
| ATOM | 268 | CB | ARG | A | 39 | 124.676 | −59.482 | 79.901 | 1.00 | 61.25 | | C |
| ANISOU | 268 | CB | ARG | A | 39 | 4749 | 6651 | 11873 | −1174 | 3703 | −1558 | C |
| ATOM | 269 | CG | ARG | A | 39 | 126.011 | −59.595 | 80.625 | 1.00 | 69.74 | | C |
| ANISOU | 269 | CG | ARG | A | 39 | 5343 | 8178 | 12976 | −1390 | 3614 | −2004 | C |
| ATOM | 270 | CD | ARG | A | 39 | 126.977 | −60.575 | 79.977 | 1.00 | 85.12 | | C |
| ANISOU | 270 | CD | ARG | A | 39 | 7103 | 10357 | 14882 | −1247 | 3479 | −1929 | C |
| ATOM | 271 | NE | ARG | A | 39 | 126.657 | −61.985 | 80.217 | 1.00 | 99.61 | | N |
| ANISOU | 271 | NE | ARG | A | 39 | 8937 | 12458 | 16451 | −876 | 3061 | −1630 | N |
| ATOM | 272 | CZ | ARG | A | 39 | 126.833 | −62.623 | 81.372 | 1.00 | 121.83 | | C |
| ANISOU | 272 | CZ | ARG | A | 39 | 11488 | 15753 | 19049 | −739 | 2741 | −1716 | C |
| ATOM | 273 | NH1 | ARG | A | 39 | 127.289 | −61.971 | 82.438 | 1.00 | 110.32 | | N |
| ANISOU | 273 | NH1 | ARG | A | 39 | 9696 | 14656 | 17562 | −954 | 2723 | −2132 | N |
| ATOM | 274 | NH2 | ARG | A | 39 | 126.539 | −63.911 | 81.477 | 1.00 | 114.59 | | N |
| ANISOU | 274 | NH2 | ARG | A | 39 | 10640 | 14967 | 17932 | −379 | 2481 | −1398 | N |
| ATOM | 275 | N | GLY | A | 40 | 124.630 | −56.320 | 81.242 | 1.00 | 64.38 | | N |
| ANISOU | 275 | N | GLY | A | 40 | 5160 | 6543 | 12758 | −1962 | 4607 | −2362 | N |
| ATOM | 276 | CA | GLY | A | 40 | 125.092 | −54.944 | 81.137 | 1.00 | 65.46 | | C |
| ANISOU | 276 | CA | GLY | A | 40 | 5336 | 6290 | 13244 | −2356 | 5256 | −2719 | C |
| ATOM | 277 | C | GLY | A | 40 | 124.019 | −53.885 | 81.278 | 1.00 | 73.25 | | C |
| ANISOU | 277 | C | GLY | A | 40 | 6715 | 6768 | 14347 | −2357 | 5653 | −2595 | C |
| ATOM | 278 | O | GLY | A | 40 | 124.328 | −52.687 | 81.253 | 1.00 | 74.60 | | O |
| ANISOU | 278 | O | GLY | A | 40 | 6972 | 6529 | 14844 | −2687 | 6295 | −2892 | O |
| ATOM | 279 | N | GLN | A | 41 | 122.758 | −54.314 | 81.398 | 1.00 | 70.40 | | N |
| ANISOU | 279 | N | GLN | A | 41 | 6600 | 6413 | 13737 | −1987 | 5331 | −2162 | N |
| ATOM | 280 | CA | GLN | A | 41 | 121.620 | −53.419 | 81.549 | 1.00 | 70.48 | | C |
| ANISOU | 280 | CA | GLN | A | 41 | 6976 | 6008 | 13795 | −1894 | 5647 | −1976 | C |
| ATOM | 281 | C | GLN | A | 41 | 121.409 | −53.121 | 83.022 | 1.00 | 76.15 | | C |
| ANISOU | 281 | C | GLN | A | 41 | 7501 | 6942 | 14489 | −2152 | 5508 | −2385 | C |
| ATOM | 282 | O | GLN | A | 41 | 121.608 | −53.995 | 83.878 | 1.00 | 76.78 | | O |
| ANISOU | 282 | O | GLN | A | 41 | 7250 | 7584 | 14341 | −2162 | 4950 | −2554 | O |
| ATOM | 283 | CB | GLN | A | 41 | 120.330 | −54.035 | 80.967 | 1.00 | 71.67 | | C |
| ANISOU | 283 | CB | GLN | A | 41 | 7427 | 6153 | 13654 | −1374 | 5356 | −1357 | C |
| ATOM | 284 | CG | GLN | A | 41 | 120.454 | −54.534 | 79.531 | 1.00 | 88.91 | | C |
| ANISOU | 284 | CG | GLN | A | 41 | 9744 | 8277 | 15761 | −1077 | 5384 | −962 | C |
| ATOM | 285 | CD | GLN | A | 41 | 119.126 | −54.649 | 78.821 | 1.00 | 98.64 | | C |
| ANISOU | 285 | CD | GLN | A | 41 | 11293 | 9441 | 16743 | −605 | 5342 | −425 | C |
| ATOM | 286 | OE1 | GLN | A | 41 | 118.061 | −54.686 | 79.433 | 1.00 | 89.99 | | O |
| ANISOU | 286 | OE1 | GLN | A | 41 | 10278 | 8434 | 15480 | −467 | 5139 | −318 | O |
| ATOM | 287 | NE2 | GLN | A | 41 | 119.159 | −54.711 | 77.502 | 1.00 | 91.31 | | N |
| ANISOU | 287 | NE2 | GLN | A | 41 | 10522 | 8415 | 15754 | −336 | 5535 | −93 | N |
| ATOM | 288 | N | LEU | A | 42 | 120.977 | −51.884 | 83.309 | 1.00 | 71.80 | | N |
| ANISOU | 288 | N | LEU | A | 42 | 7179 | 5940 | 14163 | −2324 | 6056 | −2518 | N |
| ATOM | 289 | CA | LEU | A | 42 | 120.675 | −51.434 | 84.652 | 1.00 | 70.81 | | C |
| ANISOU | 289 | CA | LEU | A | 42 | 6919 | 5950 | 14035 | −2583 | 6021 | −2917 | C |
| ATOM | 290 | C | LEU | A | 42 | 119.231 | −50.941 | 84.757 | 1.00 | 79.48 | | C |
| ANISOU | 290 | C | LEU | A | 42 | 8435 | 6689 | 15074 | −2295 | 6160 | −2527 | C |
| ATOM | 291 | O | LEU | A | 42 | 118.606 | −50.578 | 83.754 | 1.00 | 79.78 | | O |
| ANISOU | 291 | O | LEU | A | 42 | 8874 | 6287 | 15151 | −1961 | 6496 | −2032 | O |
| ATOM | 292 | CB | LEU | A | 42 | 121.650 | −50.336 | 85.090 | 1.00 | 69.62 | | C |
| ANISOU | 292 | CB | LEU | A | 42 | 6573 | 5630 | 14251 | −3182 | 6623 | −3623 | C |
| ATOM | 293 | CG | LEU | A | 42 | 123.124 | −50.688 | 85.187 | 1.00 | 72.86 | | C |
| ANISOU | 293 | CG | LEU | A | 42 | 6467 | 6505 | 14710 | −3540 | 6514 | −4155 | C |
| ATOM | 294 | CD1 | LEU | A | 42 | 123.890 | −49.571 | 85.796 | 1.00 | 73.22 | | C |
| ANISOU | 294 | CD1 | LEU | A | 42 | 6279 | 6455 | 15088 | −4183 | 7109 | −4950 | C |
| ATOM | 295 | CD2 | LEU | A | 42 | 123.361 | −51.929 | 86.002 | 1.00 | 73.52 | | C |
| ANISOU | 295 | CD2 | LEU | A | 42 | 6106 | 7438 | 14390 | −3406 | 5683 | −4237 | C |
| ATOM | 296 | N | ARG | A | 43 | 118.701 | −50.945 | 85.981 | 1.00 | 77.97 | | N |
| ANISOU | 296 | N | ARG | A | 43 | 8128 | 6747 | 14751 | −2387 | 5894 | −2741 | N |
| ATOM | 297 | CA | ARG | A | 43 | 117.372 | −50.454 | 86.264 | 1.00 | 78.92 | | C |
| ANISOU | 297 | CA | ARG | A | 43 | 8588 | 6584 | 14815 | −2158 | 6014 | −2451 | C |
| ATOM | 298 | C | ARG | A | 43 | 117.523 | −48.945 | 86.640 | 1.00 | 90.36 | | C |
| ANISOU | 298 | C | ARG | A | 43 | 10183 | 7501 | 16648 | −2544 | 6817 | −2857 | C |
| ATOM | 299 | O | ARG | A | 43 | 118.649 | −48.403 | 86.557 | 1.00 | 89.43 | | O |
| ANISOU | 299 | O | ARG | A | 43 | 9897 | 7254 | 16830 | −2983 | 7253 | −3343 | O |
| ATOM | 300 | CB | ARG | A | 43 | 116.788 | −51.273 | 87.420 | 1.00 | 75.66 | | C |
| ANISOU | 300 | CB | ARG | A | 43 | 7965 | 6710 | 14073 | −2080 | 5341 | −2500 | C |
| ATOM | 301 | CG | ARG | A | 43 | 116.200 | −52.605 | 87.001 | 1.00 | 74.85 | | C |
| ANISOU | 301 | CG | ARG | A | 43 | 7883 | 6927 | 13631 | −1636 | 4729 | −1999 | C |
| ATOM | 302 | CD | ARG | A | 43 | 115.059 | −53.009 | 87.918 | 1.00 | 78.22 | | C |
| ANISOU | 302 | CD | ARG | A | 43 | 8347 | 7574 | 13798 | −1456 | 4340 | −1865 | C |
| ATOM | 303 | NE | ARG | A | 43 | 113.748 | −52.893 | 87.263 | 1.00 | 90.58 | | N |
| ANISOU | 303 | NE | ARG | A | 43 | 10275 | 8875 | 15266 | −1063 | 4422 | −1347 | N |
| ATOM | 304 | CZ | ARG | A | 43 | 112.584 | −52.703 | 87.897 | 1.00 | 105.58 | | C |
| ANISOU | 304 | CZ | ARG | A | 43 | 12315 | 10760 | 17040 | −923 | 4349 | −1225 | C |
| ATOM | 305 | NH1 | ARG | A | 43 | 112.546 | −52.594 | 89.226 | 1.00 | 81.43 | | N |
| ANISOU | 305 | NH1 | ARG | A | 43 | 9096 | 7893 | 13949 | −1151 | 4200 | −1572 | N |
| ATOM | 306 | NH2 | ARG | A | 43 | 111.452 | −52.609 | 87.205 | 1.00 | 94.51 | | N |
| ANISOU | 306 | NH2 | ARG | A | 43 | 11187 | 9208 | 15516 | −538 | 4427 | −768 | N |
| ATOM | 307 | N | ARG | A | 44 | 116.400 | −48.261 | 87.053 | 1.00 | 91.58 | | N |
| ANISOU | 307 | N | ARG | A | 44 | 10646 | 7335 | 16814 | −2401 | 7064 | −2689 | N |
| ATOM | 308 | CA | ARG | A | 44 | 116.469 | −46.860 | 87.502 | 1.00 | 92.84 | | C |
| ANISOU | 308 | CA | ARG | A | 44 | 10971 | 6953 | 17351 | −2770 | 7872 | −3089 | C |
| ATOM | 309 | C | ARG | A | 44 | 117.346 | −46.865 | 88.769 | 1.00 | 96.68 | | C |
| ANISOU | 309 | C | ARG | A | 44 | 10947 | 7909 | 17879 | −3385 | 7694 | −3939 | C |
| ATOM | 310 | O | ARG | A | 44 | 118.260 | −46.040 | 88.875 | 1.00 | 96.53 | | O |
| ANISOU | 310 | O | ARG | A | 44 | 10807 | 7658 | 18215 | −3910 | 8301 | −4535 | O |
| ATOM | 311 | CB | ARG | A | 44 | 115.074 | −46.251 | 87.747 | 1.00 | 96.67 | | C |
| ANISOU | 311 | CB | ARG | A | 44 | 11859 | 7077 | 17793 | −2442 | 8108 | −2717 | C |
| ATOM | 312 | CG | ARG | A | 44 | 114.987 | −44.744 | 87.399 | 1.00 | 109.03 | | C |
| ANISOU | 312 | CG | ARG | A | 44 | 13859 | 7775 | 19794 | −2529 | 9214 | −2733 | C |
| ATOM | 313 | CD | ARG | A | 44 | 114.543 | −44.476 | 85.970 | 1.00 | 115.60 | | C |
| ANISOU | 313 | CD | ARG | A | 44 | 15147 | 8144 | 20630 | −1939 | 9646 | −1977 | C |
| ATOM | 314 | N | SER | A | 45 | 117.145 | −47.880 | 89.662 | 1.00 | 91.88 | | N |
| ANISOU | 314 | N | SER | A | 45 | 10001 | 8027 | 16880 | −3303 | 6862 | −3999 | N |
| ATOM | 315 | CA | SER | A | 45 | 118.025 | −48.139 | 90.798 | 1.00 | 91.50 | | C |
| ANISOU | 315 | CA | SER | A | 45 | 9388 | 8651 | 16726 | −3745 | 6544 | −4721 | C |
| ATOM | 316 | C | SER | A | 45 | 119.277 | −48.730 | 90.152 | 1.00 | 97.80 | | C |
| ANISOU | 316 | C | SER | A | 45 | 9872 | 9755 | 17534 | −3843 | 6396 | −4852 | C |
| ATOM | 317 | O | SER | A | 45 | 119.151 | −49.511 | 89.202 | 1.00 | 99.20 | | O |
| ANISOU | 317 | O | SER | A | 45 | 10202 | 9900 | 17588 | −3429 | 6117 | −4274 | O |
| ATOM | 318 | CB | SER | A | 45 | 117.403 | −49.173 | 91.723 | 1.00 | 93.52 | | C |
| ANISOU | 318 | CB | SER | A | 45 | 9454 | 9555 | 16523 | −3462 | 5730 | −4554 | C |
| ATOM | 319 | OG | SER | A | 45 | 116.942 | −50.292 | 90.987 | 1.00 | 100.32 | | O |
| ANISOU | 319 | OG | SER | A | 45 | 10463 | 10517 | 17138 | −2935 | 5232 | −3869 | O |
| ATOM | 320 | N | GLY | A | 46 | 120.458 | −48.353 | 90.631 | 1.00 | 93.98 | | N |
| ANISOU | 320 | N | GLY | A | 46 | 8937 | 9583 | 17189 | −4391 | 6603 | −5628 | N |
| ATOM | 321 | CA | GLY | A | 46 | 121.737 | −48.806 | 90.072 | 1.00 | 93.66 | | C |
| ANISOU | 321 | CA | GLY | A | 46 | 8545 | 9864 | 17178 | −4535 | 6523 | −5843 | C |
| ATOM | 322 | C | GLY | A | 46 | 121.911 | −50.299 | 89.828 | 1.00 | 96.16 | | C |
| ANISOU | 322 | C | GLY | A | 46 | 8669 | 10771 | 17095 | −4074 | 5716 | −5398 | C |
| ATOM | 323 | O | GLY | A | 46 | 122.857 | −50.711 | 89.150 | 1.00 | 96.50 | | O |
| ANISOU | 323 | O | GLY | A | 46 | 8517 | 10972 | 17177 | −4097 | 5679 | −5429 | O |
| ATOM | 324 | N | GLU | A | 47 | 120.997 | −51.105 | 90.398 | 1.00 | 90.73 | | N |
| ANISOU | 324 | N | GLU | A | 47 | 8048 | 10382 | 16042 | −3665 | 5122 | −4997 | N |
| ATOM | 325 | CA | GLU | A | 47 | 120.889 | −52.564 | 90.342 | 1.00 | 89.48 | | C |
| ANISOU | 325 | CA | GLU | A | 47 | 7789 | 10710 | 15498 | −3186 | 4403 | −4529 | C |
| ATOM | 326 | C | GLU | A | 47 | 120.875 | −53.101 | 88.902 | 1.00 | 89.57 | | C |
| ANISOU | 326 | C | GLU | A | 47 | 8095 | 10352 | 15587 | −2867 | 4415 | −3950 | C |
| ATOM | 327 | O | GLU | A | 47 | 120.230 | −52.471 | 88.053 | 1.00 | 90.08 | | O |
| ANISOU | 327 | O | GLU | A | 47 | 8609 | 9745 | 15872 | −2782 | 4834 | −3623 | O |
| ATOM | 328 | CB | GLU | A | 47 | 119.590 | −53.016 | 91.050 | 1.00 | 90.89 | | C |
| ANISOU | 328 | CB | GLU | A | 47 | 8175 | 10961 | 15399 | −2843 | 4018 | −4154 | C |
| ATOM | 329 | CG | GLU | A | 47 | 119.510 | −52.694 | 92.534 | 1.00 | 102.92 | | C |
| ANISOU | 329 | CG | GLU | A | 47 | 9403 | 12948 | 16754 | −3065 | 3897 | −4651 | C |
| ATOM | 330 | CD | GLU | A | 47 | 120.402 | −53.506 | 93.456 | 1.00 | 130.96 | | C |
| ANISOU | 330 | CD | GLU | A | 47 | 12369 | 17455 | 19937 | −3041 | 3406 | −4984 | C |
| ATOM | 331 | OE1 | GLU | A | 47 | 120.190 | −54.736 | 93.576 | 1.00 | 129.37 | | O |
| ANISOU | 331 | OE1 | GLU | A | 47 | 12147 | 17600 | 19407 | −2583 | 2891 | −4540 | O |
| ATOM | 332 | OE2 | GLU | A | 47 | 121.305 | −52.900 | 94.077 | 1.00 | 128.62 | | O |
| ANISOU | 332 | OE2 | GLU | A | 47 | 11621 | 17583 | 19666 | −3472 | 3576 | −5708 | O |
| ATOM | 333 | N | PRO | A | 48 | 121.537 | −54.271 | 88.622 | 1.00 | 81.23 | | N |
| ANISOU | 333 | N | PRO | A | 48 | 6793 | 9749 | 14320 | −2644 | 3976 | −3796 | N |
| ATOM | 334 | CA | PRO | A | 48 | 121.469 | −54.862 | 87.268 | 1.00 | 79.17 | | C |
| ANISOU | 334 | CA | PRO | A | 48 | 6804 | 9175 | 14104 | −2339 | 3964 | −3263 | C |
| ATOM | 335 | C | PRO | A | 48 | 120.047 | −55.290 | 86.936 | 1.00 | 78.39 | | C |
| ANISOU | 335 | C | PRO | A | 48 | 7131 | 8790 | 13862 | −1928 | 3787 | −2654 | C |
| ATOM | 336 | O | PRO | A | 48 | 119.307 | −55.720 | 87.837 | 1.00 | 78.74 | | O |
| ANISOU | 336 | O | PRO | A | 48 | 7166 | 9079 | 13674 | −1779 | 3447 | −2574 | O |
| ATOM | 337 | CB | PRO | A | 48 | 122.376 | −56.084 | 87.367 | 1.00 | 81.24 | | C |
| ANISOU | 337 | CB | PRO | A | 48 | 6685 | 10054 | 14129 | −2180 | 3503 | −3274 | C |
| ATOM | 338 | CG | PRO | A | 48 | 122.377 | −56.443 | 88.810 | 1.00 | 86.09 | | C |
| ANISOU | 338 | CG | PRO | A | 48 | 6963 | 11303 | 14444 | −2160 | 3125 | −3532 | C |
| ATOM | 339 | CD | PRO | A | 48 | 122.310 | −55.136 | 89.538 | 1.00 | 82.02 | | C |
| ANISOU | 339 | CD | PRO | A | 48 | 6374 | 10692 | 14100 | −2599 | 3487 | −4064 | C |
| ATOM | 340 | N | TYR | A | 49 | 119.650 | −55.150 | 85.660 | 1.00 | 69.49 | | N |
| ANISOU | 340 | N | TYR | A | 49 | 6356 | 7195 | 12853 | −1742 | 4035 | −2247 | N |
| ATOM | 341 | CA | TYR | A | 49 | 118.281 | −55.449 | 85.240 | 1.00 | 66.15 | | C |
| ANISOU | 341 | CA | TYR | A | 49 | 6290 | 6569 | 12276 | −1367 | 3917 | −1727 | C |
| ATOM | 342 | C | TYR | A | 49 | 117.824 | −56.837 | 85.616 | 1.00 | 61.91 | | C |
| ANISOU | 342 | C | TYR | A | 49 | 5667 | 6432 | 11427 | −1096 | 3340 | −1504 | C |
| ATOM | 343 | O | TYR | A | 49 | 116.735 | −56.945 | 86.142 | 1.00 | 60.02 | | O |
| ANISOU | 343 | O | TYR | A | 49 | 5564 | 6200 | 11040 | −953 | 3186 | −1349 | O |
| ATOM | 344 | CB | TYR | A | 49 | 118.066 | −55.183 | 83.752 | 1.00 | 67.71 | | C |
| ANISOU | 344 | CB | TYR | A | 49 | 6791 | 6365 | 12569 | −1163 | 4245 | −1348 | C |
| ATOM | 345 | CG | TYR | A | 49 | 116.624 | −54.924 | 83.405 | 1.00 | 69.59 | | C |
| ANISOU | 345 | CG | TYR | A | 49 | 7377 | 6384 | 12682 | −837 | 4317 | −931 | C |
| ATOM | 346 | CD1 | TYR | A | 49 | 116.097 | −53.640 | 83.458 | 1.00 | 72.43 | | C |
| ANISOU | 346 | CD1 | TYR | A | 49 | 7992 | 6326 | 13200 | −856 | 4819 | −907 | C |
| ATOM | 347 | CD2 | TYR | A | 49 | 115.783 | −55.961 | 83.025 | 1.00 | 70.85 | | C |
| ANISOU | 347 | CD2 | TYR | A | 49 | 7591 | 6770 | 12558 | −507 | 3924 | −583 | C |
| ATOM | 348 | CE1 | TYR | A | 49 | 114.751 | −53.399 | 83.186 | 1.00 | 75.04 | | C |
| ANISOU | 348 | CE1 | TYR | A | 49 | 8605 | 6538 | 13369 | −498 | 4876 | −510 | C |
| ATOM | 349 | CE2 | TYR | A | 49 | 114.436 | −55.733 | 82.741 | 1.00 | 72.66 | | C |
| ANISOU | 349 | CE2 | TYR | A | 49 | 8066 | 6915 | 12627 | −209 | 3969 | −253 | C |
| ATOM | 350 | CZ | TYR | A | 49 | 113.926 | −54.448 | 82.819 | 1.00 | 82.50 | | C |
| ANISOU | 350 | CZ | TYR | A | 49 | 9544 | 7808 | 13994 | −175 | 4425 | −195 | C |
| ATOM | 351 | OH | TYR | A | 49 | 112.607 | −54.219 | 82.531 | 1.00 | 85.61 | | O |
| ANISOU | 351 | OH | TYR | A | 49 | 10149 | 8189 | 14191 | 179 | 4470 | 150 | O |
| ATOM | 352 | N | ILE | A | 50 | 118.665 | −57.880 | 85.452 | 1.00 | 53.98 | | N |
| ANISOU | 352 | N | ILE | A | 50 | 4430 | 5751 | 10330 | −1033 | 3065 | −1511 | N |
| ATOM | 353 | CA | ILE | A | 50 | 118.273 | −59.248 | 85.811 | 1.00 | 50.93 | | C |
| ANISOU | 353 | CA | ILE | A | 50 | 4000 | 5675 | 9678 | −761 | 2621 | −1287 | C |
| ATOM | 354 | C | ILE | A | 50 | 117.597 | −59.372 | 87.210 | 1.00 | 58.21 | | C |
| ANISOU | 354 | C | ILE | A | 50 | 4869 | 6832 | 10417 | −728 | 2385 | −1364 | C |
| ATOM | 355 | O | ILE | A | 50 | 116.743 | −60.255 | 87.382 | 1.00 | 60.90 | | O |
| ANISOU | 355 | O | ILE | A | 50 | 5335 | 7225 | 10578 | −488 | 2154 | −1092 | O |
| ATOM | 356 | CB | ILE | A | 50 | 119.420 | −60.257 | 85.666 | 1.00 | 51.21 | | C |
| ANISOU | 356 | CB | ILE | A | 50 | 3768 | 6040 | 9651 | −691 | 2428 | −1328 | C |
| ATOM | 357 | CG1 | ILE | A | 50 | 118.919 | −61.727 | 85.702 | 1.00 | 48.16 | | C |
| ANISOU | 357 | CG1 | ILE | A | 50 | 3453 | 5798 | 9047 | −365 | 2121 | −1003 | C |
| ATOM | 358 | CG2 | ILE | A | 50 | 120.527 | −59.990 | 86.691 | 1.00 | 52.04 | | C |
| ANISOU | 358 | CG2 | ILE | A | 50 | 3461 | 6584 | 9729 | −886 | 2368 | −1767 | C |
| ATOM | 359 | CD1 | ILE | A | 50 | 118.088 | −62.158 | 84.557 | 1.00 | 31.11 | | C |
| ANISOU | 359 | CD1 | ILE | A | 50 | 1584 | 3345 | 6893 | −217 | 2180 | −680 | C |
| ATOM | 360 | N | THR | A | 51 | 117.922 | −58.489 | 88.182 | 1.00 | 51.71 | | N |
| ANISOU | 360 | N | THR | A | 51 | 3866 | 6140 | 9641 | −984 | 2480 | −1754 | N |
| ATOM | 361 | CA | THR | A | 51 | 117.322 | −58.620 | 89.502 | 1.00 | 50.98 | | C |
| ANISOU | 361 | CA | THR | A | 51 | 3708 | 6314 | 9346 | −937 | 2254 | −1830 | C |
| ATOM | 362 | C | THR | A | 51 | 115.788 | −58.565 | 89.415 | 1.00 | 53.47 | | C |
| ANISOU | 362 | C | THR | A | 51 | 4387 | 6310 | 9620 | −774 | 2258 | −1504 | C |
| ATOM | 363 | O | THR | A | 51 | 115.106 | −59.312 | 90.135 | 1.00 | 53.20 | | O |
| ANISOU | 363 | O | THR | A | 51 | 4378 | 6464 | 9372 | −585 | 1989 | −1354 | O |
| ATOM | 364 | CB | THR | A | 51 | 117.917 | −57.635 | 90.515 | 1.00 | 65.98 | | C |
| ANISOU | 364 | CB | THR | A | 51 | 5327 | 8451 | 11292 | −1274 | 2382 | −2370 | C |
| ATOM | 365 | OG1 | THR | A | 51 | 117.496 | −56.314 | 90.209 | 1.00 | 79.83 | | O |
| ANISOU | 365 | OG1 | THR | A | 51 | 7305 | 9710 | 13317 | −1523 | 2820 | −2497 | O |
| ATOM | 366 | CG2 | THR | A | 51 | 119.415 | −57.695 | 90.597 | 1.00 | 60.46 | | C |
| ANISOU | 366 | CG2 | THR | A | 51 | 4198 | 8177 | 10598 | −1445 | 2374 | −2752 | C |
| ATOM | 367 | N | HIS | A | 52 | 115.261 | −57.700 | 88.501 | 1.00 | 47.59 | | N |
| ANISOU | 367 | N | HIS | A | 52 | 3913 | 5105 | 9062 | −812 | 2592 | −1378 | N |
| ATOM | 368 | CA | HIS | A | 52 | 113.831 | −57.505 | 88.256 | 1.00 | 45.06 | | C |
| ANISOU | 368 | CA | HIS | A | 52 | 3901 | 4533 | 8686 | −628 | 2643 | −1074 | C |
| ATOM | 369 | C | HIS | A | 52 | 113.199 | −58.814 | 87.722 | 1.00 | 49.13 | | C |
| ANISOU | 369 | C | HIS | A | 52 | 4495 | 5162 | 9009 | −345 | 2362 | −731 | C |
| ATOM | 370 | O | HIS | A | 52 | 112.430 | −59.400 | 88.490 | 1.00 | 50.31 | | O |
| ANISOU | 370 | O | HIS | A | 52 | 4662 | 5465 | 8990 | −243 | 2135 | −663 | O |
| ATOM | 371 | CB | HIS | A | 52 | 113.539 | −56.269 | 87.370 | 1.00 | 43.61 | | C |
| ANISOU | 371 | CB | HIS | A | 52 | 3966 | 3898 | 8706 | −649 | 3117 | −986 | C |
| ATOM | 372 | CG | HIS | A | 52 | 112.113 | −56.175 | 86.905 | 1.00 | 45.22 | | C |
| ANISOU | 372 | CG | HIS | A | 52 | 4442 | 3957 | 8785 | −360 | 3154 | −620 | C |
| ATOM | 373 | ND1 | HIS | A | 52 | 111.069 | −56.011 | 87.800 | 1.00 | 45.37 | | N |
| ANISOU | 373 | ND1 | HIS | A | 52 | 4533 | 4016 | 8690 | −309 | 3057 | −605 | N |
| ATOM | 374 | CD2 | HIS | A | 52 | 111.609 | −56.202 | 85.648 | 1.00 | 45.75 | | C |
| ANISOU | 374 | CD2 | HIS | A | 52 | 4679 | 3911 | 8793 | −99 | 3281 | −286 | C |
| ATOM | 375 | CE1 | HIS | A | 52 | 109.973 | −55.939 | 87.062 | 1.00 | 44.22 | | C |
| ANISOU | 375 | CE1 | HIS | A | 52 | 4584 | 3792 | 8428 | −24 | 3127 | −273 | C |
| ATOM | 376 | NE2 | HIS | A | 52 | 110.241 | −56.071 | 85.766 | 1.00 | 44.69 | | N |
| ANISOU | 376 | NE2 | HIS | A | 52 | 4693 | 3797 | 8489 | 123 | 3248 | −75 | N |
| ATOM | 377 | N | PRO | A | 53 | 113.531 | −59.359 | 86.515 | 1.00 | 42.23 | | N |
| ANISOU | 377 | N | PRO | A | 53 | 3650 | 4239 | 8155 | −241 | 2387 | −558 | N |
| ATOM | 378 | CA | PRO | A | 53 | 112.885 | −60.597 | 86.085 | 1.00 | 42.56 | | C |
| ANISOU | 378 | CA | PRO | A | 53 | 3747 | 4393 | 8029 | −37 | 2175 | −326 | C |
| ATOM | 379 | C | PRO | A | 53 | 113.018 | −61.778 | 87.070 | 1.00 | 48.19 | | C |
| ANISOU | 379 | C | PRO | A | 53 | 4337 | 5362 | 8609 | 16 | 1893 | −355 | C |
| ATOM | 380 | O | PRO | A | 53 | 112.103 | −62.606 | 87.116 | 1.00 | 48.90 | | O |
| ANISOU | 380 | O | PRO | A | 53 | 4524 | 5483 | 8574 | 141 | 1788 | −209 | O |
| ATOM | 381 | CB | PRO | A | 53 | 113.538 | −60.858 | 84.721 | 1.00 | 44.21 | | C |
| ANISOU | 381 | CB | PRO | A | 53 | 3955 | 4533 | 8308 | 4 | 2289 | −232 | C |
| ATOM | 382 | CG | PRO | A | 53 | 113.850 | −59.520 | 84.197 | 1.00 | 47.33 | | C |
| ANISOU | 382 | CG | PRO | A | 53 | 4430 | 4682 | 8872 | −78 | 2628 | −269 | C |
| ATOM | 383 | CD | PRO | A | 53 | 114.385 | −58.841 | 85.434 | 1.00 | 42.63 | | C |
| ANISOU | 383 | CD | PRO | A | 53 | 3706 | 4106 | 8384 | −310 | 2659 | −573 | C |
| ATOM | 384 | N | VAL | A | 54 | 114.120 | −61.847 | 87.882 | 1.00 | 43.59 | | N |
| ANISOU | 384 | N | VAL | A | 54 | 3532 | 4994 | 8035 | −58 | 1807 | −550 | N |
| ATOM | 385 | CA | VAL | A | 54 | 114.303 | −62.903 | 88.907 | 1.00 | 42.21 | | C |
| ANISOU | 385 | CA | VAL | A | 54 | 3240 | 5112 | 7686 | 90 | 1583 | −525 | C |
| ATOM | 386 | C | VAL | A | 54 | 113.251 | −62.684 | 90.014 | 1.00 | 48.40 | | C |
| ANISOU | 386 | C | VAL | A | 54 | 4107 | 5937 | 8347 | 111 | 1503 | −528 | C |
| ATOM | 387 | O | VAL | A | 54 | 112.530 | −63.624 | 90.363 | 1.00 | 49.00 | | O |
| ANISOU | 387 | O | VAL | A | 54 | 4290 | 6031 | 8298 | 276 | 1416 | −350 | O |
| ATOM | 388 | CB | VAL | A | 54 | 115.742 | −62.967 | 89.485 | 1.00 | 44.38 | | C |
| ANISOU | 388 | CB | VAL | A | 54 | 3196 | 5743 | 7924 | 73 | 1506 | −733 | C |
| ATOM | 389 | CG1 | VAL | A | 54 | 115.841 | −63.935 | 90.656 | 1.00 | 42.73 | | C |
| ANISOU | 389 | CG1 | VAL | A | 54 | 2873 | 5895 | 7469 | 324 | 1310 | −653 | C |
| ATOM | 390 | CG2 | VAL | A | 54 | 116.749 | −63.350 | 88.408 | 1.00 | 44.57 | | C |
| ANISOU | 390 | CG2 | VAL | A | 54 | 3139 | 5737 | 8059 | 83 | 1579 | −702 | C |
| ATOM | 391 | N | ALA | A | 55 | 113.126 | −61.433 | 90.521 | 1.00 | 43.84 | | N |
| ANISOU | 391 | N | ALA | A | 55 | 3500 | 5329 | 7827 | −73 | 1586 | −742 | N |
| ATOM | 392 | CA | ALA | A | 55 | 112.153 | −61.066 | 91.552 | 1.00 | 42.88 | | C |
| ANISOU | 392 | CA | ALA | A | 55 | 3455 | 5234 | 7604 | −77 | 1536 | −775 | C |
| ATOM | 393 | C | ALA | A | 55 | 110.697 | −61.253 | 91.128 | 1.00 | 43.95 | | C |
| ANISOU | 393 | C | ALA | A | 55 | 3854 | 5134 | 7712 | 26 | 1566 | −532 | C |
| ATOM | 394 | O | ALA | A | 55 | 109.843 | −61.492 | 91.988 | 1.00 | 44.36 | | O |
| ANISOU | 394 | O | ALA | A | 55 | 3968 | 5251 | 7635 | 94 | 1471 | −487 | O |
| ATOM | 395 | CB | ALA | A | 55 | 112.383 | −59.646 | 91.994 | 1.00 | 43.86 | | C |
| ANISOU | 395 | CB | ALA | A | 55 | 3508 | 5312 | 7843 | −328 | 1704 | −1085 | C |
| ATOM | 396 | N | VAL | A | 56 | 110.413 | −61.117 | 89.824 | 1.00 | 37.22 | | N |
| ANISOU | 396 | N | VAL | A | 56 | 3126 | 4064 | 6953 | 46 | 1704 | −397 | N |
| ATOM | 397 | CA | VAL | A | 56 | 109.093 | −61.362 | 89.250 | 1.00 | 36.15 | | C |
| ANISOU | 397 | CA | VAL | A | 56 | 3159 | 3836 | 6740 | 161 | 1727 | −207 | C |
| ATOM | 398 | C | VAL | A | 56 | 108.855 | −62.898 | 89.217 | 1.00 | 41.18 | | C |
| ANISOU | 398 | C | VAL | A | 56 | 3795 | 4579 | 7274 | 261 | 1596 | −103 | C |
| ATOM | 399 | O | VAL | A | 56 | 107.778 | −63.369 | 89.640 | 1.00 | 40.63 | | O |
| ANISOU | 399 | O | VAL | A | 56 | 3802 | 4537 | 7099 | 309 | 1554 | −51 | O |
| ATOM | 400 | CB | VAL | A | 56 | 108.970 | −60.695 | 87.873 | 1.00 | 39.42 | | C |
| ANISOU | 400 | CB | VAL | A | 56 | 3658 | 4095 | 7227 | 200 | 1931 | −107 | C |
| ATOM | 401 | CG1 | VAL | A | 56 | 107.679 | −61.069 | 87.173 | 1.00 | 38.75 | | C |
| ANISOU | 401 | CG1 | VAL | A | 56 | 3658 | 4078 | 6989 | 351 | 1926 | 52 | C |
| ATOM | 402 | CG2 | VAL | A | 56 | 109.040 | −59.201 | 88.043 | 1.00 | 39.92 | | C |
| ANISOU | 402 | CG2 | VAL | A | 56 | 3789 | 3966 | 7413 | 127 | 2167 | −181 | C |
| ATOM | 403 | N | ALA | A | 57 | 109.895 | −63.677 | 88.791 | 1.00 | 37.20 | | N |
| ANISOU | 403 | N | ALA | A | 57 | 3204 | 4113 | 6816 | 285 | 1580 | −91 | N |
| ATOM | 404 | CA | ALA | A | 57 | 109.860 | −65.138 | 88.745 | 1.00 | 36.49 | | C |
| ANISOU | 404 | CA | ALA | A | 57 | 3139 | 4051 | 6676 | 382 | 1559 | 3 | C |
| ATOM | 405 | C | ALA | A | 57 | 109.716 | −65.676 | 90.141 | 1.00 | 42.42 | | C |
| ANISOU | 405 | C | ALA | A | 57 | 3894 | 4906 | 7319 | 480 | 1480 | 33 | C |
| ATOM | 406 | O | ALA | A | 57 | 109.051 | −66.694 | 90.305 | 1.00 | 44.00 | | O |
| ANISOU | 406 | O | ALA | A | 57 | 4202 | 5044 | 7473 | 549 | 1545 | 122 | O |
| ATOM | 407 | CB | ALA | A | 57 | 111.109 | −65.683 | 88.105 | 1.00 | 36.77 | | C |
| ANISOU | 407 | CB | ALA | A | 57 | 3082 | 4100 | 6790 | 416 | 1588 | 19 | C |
| ATOM | 408 | N | GLU | A | 58 | 110.284 | −64.972 | 91.156 | 1.00 | 38.82 | | N |
| ANISOU | 408 | N | GLU | A | 58 | 3316 | 4622 | 6813 | 478 | 1378 | −67 | N |
| ATOM | 409 | CA | GLU | A | 58 | 110.140 | −65.329 | 92.575 | 1.00 | 38.92 | | C |
| ANISOU | 409 | CA | GLU | A | 58 | 3306 | 4822 | 6661 | 612 | 1290 | −39 | C |
| ATOM | 410 | C | GLU | A | 58 | 108.695 | −65.187 | 93.031 | 1.00 | 36.97 | | C |
| ANISOU | 410 | C | GLU | A | 58 | 3221 | 4463 | 6363 | 578 | 1308 | −8 | C |
| ATOM | 411 | O | GLU | A | 58 | 108.173 | −66.121 | 93.649 | 1.00 | 33.65 | | O |
| ANISOU | 411 | O | GLU | A | 58 | 2899 | 4037 | 5849 | 714 | 1346 | 120 | O |
| ATOM | 412 | CB | GLU | A | 58 | 111.086 | −64.525 | 93.486 | 1.00 | 41.31 | | C |
| ANISOU | 412 | CB | GLU | A | 58 | 3372 | 5441 | 6883 | 584 | 1174 | −237 | C |
| ATOM | 413 | CG | GLU | A | 58 | 112.433 | −65.212 | 93.661 | 1.00 | 63.87 | | C |
| ANISOU | 413 | CG | GLU | A | 58 | 6021 | 8598 | 9649 | 768 | 1118 | −214 | C |
| ATOM | 414 | CD | GLU | A | 58 | 113.391 | −64.601 | 94.667 | 1.00 | 110.85 | | C |
| ANISOU | 414 | CD | GLU | A | 58 | 11648 | 15031 | 15438 | 768 | 987 | −464 | C |
| ATOM | 415 | OE1 | GLU | A | 58 | 113.896 | −63.488 | 94.401 | 1.00 | 121.17 | | O |
| ANISOU | 415 | OE1 | GLU | A | 58 | 12802 | 16365 | 16870 | 489 | 1014 | −766 | O |
| ATOM | 416 | OE2 | GLU | A | 58 | 113.655 | −65.242 | 95.710 | 1.00 | 114.25 | | O |
| ANISOU | 416 | OE2 | GLU | A | 58 | 11965 | 15839 | 15605 | 1056 | 894 | −374 | O |
| ATOM | 417 | N | ILE | A | 59 | 108.040 | −64.065 | 92.647 | 1.00 | 33.55 | | N |
| ANISOU | 417 | N | ILE | A | 59 | 2829 | 3919 | 5998 | 421 | 1333 | −104 | N |
| ATOM | 418 | CA | ILE | A | 59 | 106.644 | −63.765 | 92.999 | 1.00 | 35.05 | | C |
| ANISOU | 418 | CA | ILE | A | 59 | 3144 | 4040 | 6133 | 397 | 1352 | −87 | C |
| ATOM | 419 | C | ILE | A | 59 | 105.700 | −64.845 | 92.455 | 1.00 | 40.92 | | C |
| ANISOU | 419 | C | ILE | A | 59 | 3997 | 4694 | 6856 | 431 | 1434 | 14 | C |
| ATOM | 420 | O | ILE | A | 59 | 104.829 | −65.334 | 93.196 | 1.00 | 41.16 | | O |
| ANISOU | 420 | O | ILE | A | 59 | 4107 | 4725 | 6806 | 463 | 1455 | 46 | O |
| ATOM | 421 | CB | ILE | A | 59 | 106.231 | −62.315 | 92.594 | 1.00 | 38.13 | | C |
| ANISOU | 421 | CB | ILE | A | 59 | 3561 | 4330 | 6595 | 291 | 1423 | −169 | C |
| ATOM | 422 | CG1 | ILE | A | 59 | 106.969 | −61.262 | 93.432 | 1.00 | 38.21 | | C |
| ANISOU | 422 | CG1 | ILE | A | 59 | 3474 | 4402 | 6642 | 188 | 1419 | −359 | C |
| ATOM | 423 | CG2 | ILE | A | 59 | 104.738 | −62.101 | 92.731 | 1.00 | 38.51 | | C |
| ANISOU | 423 | CG2 | ILE | A | 59 | 3722 | 4343 | 6568 | 318 | 1454 | −119 | C |
| ATOM | 424 | CD1 | ILE | A | 59 | 107.021 | −59.946 | 92.813 | 1.00 | 41.17 | | C |
| ANISOU | 424 | CD1 | ILE | A | 59 | 3892 | 4588 | 7165 | 81 | 1610 | −435 | C |
| ATOM | 425 | N | LEU | A | 60 | 105.919 | −65.233 | 91.184 | 1.00 | 37.51 | | N |
| ANISOU | 425 | N | LEU | A | 60 | 3553 | 4202 | 6498 | 402 | 1509 | 29 | N |
| ATOM | 426 | CA | LEU | A | 60 | 105.162 | −66.269 | 90.489 | 1.00 | 37.92 | | C |
| ANISOU | 426 | CA | LEU | A | 60 | 3654 | 4211 | 6544 | 369 | 1629 | 26 | C |
| ATOM | 427 | C | LEU | A | 60 | 105.429 | −67.651 | 91.120 | 1.00 | 45.05 | | C |
| ANISOU | 427 | C | LEU | A | 60 | 4632 | 5024 | 7460 | 441 | 1740 | 96 | C |
| ATOM | 428 | O | LEU | A | 60 | 104.473 | −68.382 | 91.373 | 1.00 | 45.50 | | O |
| ANISOU | 428 | O | LEU | A | 60 | 4775 | 5014 | 7499 | 398 | 1881 | 67 | O |
| ATOM | 429 | CB | LEU | A | 60 | 105.511 | −66.274 | 88.983 | 1.00 | 37.96 | | C |
| ANISOU | 429 | CB | LEU | A | 60 | 3595 | 4226 | 6600 | 327 | 1684 | −5 | C |
| ATOM | 430 | CG | LEU | A | 60 | 105.227 | −65.000 | 88.172 | 1.00 | 41.45 | | C |
| ANISOU | 430 | CG | LEU | A | 60 | 3994 | 4746 | 7007 | 339 | 1666 | −6 | C |
| ATOM | 431 | CD1 | LEU | A | 60 | 105.902 | −65.053 | 86.834 | 1.00 | 39.92 | | C |
| ANISOU | 431 | CD1 | LEU | A | 60 | 3743 | 4568 | 6855 | 347 | 1726 | 3 | C |
| ATOM | 432 | CD2 | LEU | A | 60 | 103.745 | −64.830 | 87.934 | 1.00 | 45.47 | | C |
| ANISOU | 432 | CD2 | LEU | A | 60 | 4492 | 5413 | 7373 | 347 | 1692 | −52 | C |
| ATOM | 433 | N | ALA | A | 61 | 106.709 | −67.981 | 91.430 | 1.00 | 43.03 | | N |
| ANISOU | 433 | N | ALA | A | 61 | 4342 | 4780 | 7227 | 575 | 1716 | 192 | N |
| ATOM | 434 | CA | ALA | A | 61 | 107.086 | −69.247 | 92.067 | 1.00 | 44.46 | | C |
| ANISOU | 434 | CA | ALA | A | 61 | 4614 | 4885 | 7394 | 754 | 1874 | 338 | C |
| ATOM | 435 | C | ALA | A | 61 | 106.497 | −69.410 | 93.486 | 1.00 | 54.10 | | C |
| ANISOU | 435 | C | ALA | A | 61 | 5924 | 6142 | 8488 | 882 | 1891 | 428 | C |
| ATOM | 436 | O | ALA | A | 61 | 106.245 | −70.542 | 93.927 | 1.00 | 55.16 | | O |
| ANISOU | 436 | O | ALA | A | 61 | 6213 | 6121 | 8623 | 1010 | 2141 | 558 | O |
| ATOM | 437 | CB | ALA | A | 61 | 108.588 | −69.387 | 92.114 | 1.00 | 45.02 | | C |
| ANISOU | 437 | CB | ALA | A | 61 | 4575 | 5075 | 7455 | 934 | 1814 | 433 | C |
| ATOM | 438 | N | GLY | A | 62 | 106.292 | −68.290 | 94.178 | 1.00 | 51.87 | | N |
| ANISOU | 438 | N | GLY | A | 62 | 5563 | 6037 | 8109 | 851 | 1679 | 358 | N |
| ATOM | 439 | CA | GLY | A | 62 | 105.688 | −68.283 | 95.502 | 1.00 | 52.82 | | C |
| ANISOU | 439 | CA | GLY | A | 62 | 5749 | 6232 | 8089 | 957 | 1669 | 417 | C |
| ATOM | 440 | C | GLY | A | 62 | 104.221 | −68.662 | 95.407 | 1.00 | 60.06 | | C |
| ANISOU | 440 | C | GLY | A | 62 | 6815 | 6952 | 9052 | 817 | 1838 | 374 | C |
| ATOM | 441 | O | GLY | A | 62 | 103.633 | −69.247 | 96.336 | 1.00 | 58.75 | | O |
| ANISOU | 441 | O | GLY | A | 62 | 6778 | 6728 | 8818 | 917 | 1979 | 468 | O |
| ATOM | 442 | N | LEU | A | 63 | 103.631 | −68.351 | 94.240 | 1.00 | 58.18 | | N |
| ANISOU | 442 | N | LEU | A | 63 | 6540 | 6654 | 8913 | 597 | 1846 | 220 | N |
| ATOM | 443 | CA | LEU | A | 63 | 102.228 | −68.638 | 93.939 | 1.00 | 57.73 | | C |
| ANISOU | 443 | CA | LEU | A | 63 | 6533 | 6528 | 8874 | 428 | 1990 | 94 | C |
| ATOM | 444 | C | LEU | A | 63 | 102.086 | −70.021 | 93.299 | 1.00 | 60.53 | | C |
| ANISOU | 444 | C | LEU | A | 63 | 6964 | 6690 | 9344 | 343 | 2308 | 42 | C |
| ATOM | 445 | O | LEU | A | 63 | 100.994 | −70.422 | 92.903 | 1.00 | 59.32 | | O |
| ANISOU | 445 | O | LEU | A | 63 | 6804 | 6517 | 9219 | 146 | 2480 | −148 | O |
| ATOM | 446 | CB | LEU | A | 63 | 101.648 | −67.552 | 93.001 | 1.00 | 57.43 | | C |
| ANISOU | 446 | CB | LEU | A | 63 | 6366 | 6642 | 8813 | 297 | 1848 | −49 | C |
| ATOM | 447 | CG | LEU | A | 63 | 101.495 | −66.104 | 93.482 | 1.00 | 61.17 | | C |
| ANISOU | 447 | CG | LEU | A | 63 | 6799 | 7230 | 9213 | 339 | 1651 | −37 | C |
| ATOM | 448 | CD1 | LEU | A | 63 | 100.256 | −65.509 | 92.915 | 1.00 | 61.41 | | C |
| ANISOU | 448 | CD1 | LEU | A | 63 | 6771 | 7388 | 9173 | 276 | 1656 | −133 | C |
| ATOM | 449 | CD2 | LEU | A | 63 | 101.492 | −65.961 | 95.002 | 1.00 | 63.12 | | C |
| ANISOU | 449 | CD2 | LEU | A | 63 | 7114 | 7481 | 9387 | 427 | 1595 | 29 | C |
| ATOM | 450 | N | GLN | A | 64 | 103.193 | −70.745 | 93.206 | 1.00 | 58.29 | | N |
| ANISOU | 450 | N | GLN | A | 64 | 6734 | 6286 | 9127 | 483 | 2415 | 180 | N |
| ATOM | 451 | CA | GLN | A | 64 | 103.279 | −72.073 | 92.606 | 1.00 | 59.16 | | C |
| ANISOU | 451 | CA | GLN | A | 64 | 6947 | 6150 | 9382 | 423 | 2784 | 145 | C |
| ATOM | 452 | C | GLN | A | 64 | 102.739 | −72.094 | 91.165 | 1.00 | 63.29 | | C |
| ANISOU | 452 | C | GLN | A | 64 | 7339 | 6737 | 9971 | 128 | 2830 | −153 | C |
| ATOM | 453 | O | GLN | A | 64 | 102.022 | −73.019 | 90.766 | 1.00 | 63.77 | | O |
| ANISOU | 453 | O | GLN | A | 64 | 7440 | 6664 | 10126 | −82 | 3167 | −363 | O |
| ATOM | 454 | CB | GLN | A | 64 | 102.653 | −73.152 | 93.500 | 1.00 | 60.77 | | C |
| ANISOU | 454 | CB | GLN | A | 64 | 7370 | 6087 | 9632 | 470 | 3180 | 216 | C |
| ATOM | 455 | CG | GLN | A | 64 | 103.631 | −73.623 | 94.569 | 1.00 | 87.32 | | C |
| ANISOU | 455 | CG | GLN | A | 64 | 10877 | 9378 | 12925 | 879 | 3266 | 581 | C |
| ATOM | 456 | CD | GLN | A | 64 | 103.177 | −74.873 | 95.275 | 1.00 | 121.36 | | C |
| ANISOU | 456 | CD | GLN | A | 64 | 15464 | 13338 | 17311 | 993 | 3796 | 719 | C |
| ATOM | 457 | OE1 | GLN | A | 64 | 102.841 | −75.890 | 94.651 | 1.00 | 123.25 | | O |
| ANISOU | 457 | OE1 | GLN | A | 64 | 15832 | 13242 | 17756 | 809 | 4254 | 584 | O |
| ATOM | 458 | NE2 | GLN | A | 64 | 103.173 | −74.828 | 96.604 | 1.00 | 114.22 | | N |
| ANISOU | 458 | NE2 | GLN | A | 64 | 14661 | 12499 | 16238 | 1302 | 3793 | 980 | N |
| ATOM | 459 | N | MET | A | 65 | 103.128 | −71.074 | 90.386 | 1.00 | 58.02 | | N |
| ANISOU | 459 | N | MET | A | 65 | 6505 | 6298 | 9244 | 118 | 2529 | −185 | N |
| ATOM | 460 | CA | MET | A | 65 | 102.717 | −70.904 | 89.003 | 1.00 | 57.41 | | C |
| ANISOU | 460 | CA | MET | A | 65 | 6267 | 6399 | 9146 | −68 | 2519 | −417 | C |
| ATOM | 461 | C | MET | A | 65 | 103.355 | −71.923 | 88.067 | 1.00 | 65.68 | | C |
| ANISOU | 461 | C | MET | A | 65 | 7323 | 7310 | 10321 | −148 | 2754 | −504 | C |
| ATOM | 462 | O | MET | A | 65 | 104.417 | −72.502 | 88.348 | 1.00 | 65.48 | | O |
| ANISOU | 462 | O | MET | A | 65 | 7417 | 7061 | 10402 | 2 | 2857 | −317 | O |
| ATOM | 463 | CB | MET | A | 65 | 103.089 | −69.506 | 88.498 | 1.00 | 58.97 | | C |
| ANISOU | 463 | CB | MET | A | 65 | 6337 | 6821 | 9247 | 24 | 2203 | −345 | C |
| ATOM | 464 | CG | MET | A | 65 | 102.247 | −68.406 | 89.045 | 1.00 | 61.29 | | C |
| ANISOU | 464 | CG | MET | A | 65 | 6597 | 7275 | 9414 | 63 | 2032 | −328 | C |
| ATOM | 465 | SD | MET | A | 65 | 100.547 | −68.433 | 88.483 | 1.00 | 63.54 | | S |
| ANISOU | 465 | SD | MET | A | 65 | 6735 | 7863 | 9546 | −86 | 2104 | −591 | S |
| ATOM | 466 | CE | MET | A | 65 | 99.849 | −67.236 | 89.605 | 1.00 | 59.15 | | C |
| ANISOU | 466 | CE | MET | A | 65 | 6221 | 7365 | 8887 | 37 | 1932 | −466 | C |
| ATOM | 467 | N | ASP | A | 66 | 102.702 | −72.066 | 86.902 | 1.00 | 64.21 | | N |
| ANISOU | 467 | N | ASP | A | 66 | 6977 | 7333 | 10089 | −362 | 2828 | −801 | N |
| ATOM | 468 | CA | ASP | A | 66 | 103.019 | −72.867 | 85.726 | 1.00 | 64.01 | | C |
| ANISOU | 468 | CA | ASP | A | 66 | 6884 | 7301 | 10137 | −516 | 3039 | −1008 | C |
| ATOM | 469 | C | ASP | A | 66 | 104.484 | −72.664 | 85.344 | 1.00 | 68.01 | | C |
| ANISOU | 469 | C | ASP | A | 66 | 7426 | 7704 | 10711 | −328 | 2918 | −771 | C |
| ATOM | 470 | O | ASP | A | 66 | 104.979 | −71.537 | 85.450 | 1.00 | 66.03 | | O |
| ANISOU | 470 | O | ASP | A | 66 | 7131 | 7576 | 10382 | −157 | 2609 | −574 | O |
| ATOM | 471 | CB | ASP | A | 66 | 102.112 | −72.315 | 84.618 | 1.00 | 66.32 | | C |
| ANISOU | 471 | CB | ASP | A | 66 | 6898 | 8084 | 10218 | −654 | 2919 | −1297 | C |
| ATOM | 472 | CG | ASP | A | 66 | 101.809 | −73.170 | 83.426 | 1.00 | 85.21 | | C |
| ANISOU | 472 | CG | ASP | A | 66 | 9122 | 10656 | 12597 | −917 | 3166 | −1699 | C |
| ATOM | 473 | OD1 | ASP | A | 66 | 102.652 | −74.039 | 83.082 | 1.00 | 90.07 | | O |
| ANISOU | 473 | OD1 | ASP | A | 66 | 9843 | 10981 | 13397 | −974 | 3399 | −1714 | O |
| ATOM | 474 | OD2 | ASP | A | 66 | 100.751 | −72.940 | 82.796 | 1.00 | 89.82 | | O |
| ANISOU | 474 | OD2 | ASP | A | 66 | 9440 | 11725 | 12961 | −1050 | 3129 | −2013 | O |
| ATOM | 475 | N | ALA | A | 67 | 105.180 | −73.745 | 84.905 | 1.00 | 67.22 | | N |
| ANISOU | 475 | N | ALA | A | 67 | 7406 | 7363 | 10771 | −371 | 3203 | −809 | N |
| ATOM | 476 | CA | ALA | A | 67 | 106.603 | −73.688 | 84.494 | 1.00 | 68.00 | | C |
| ANISOU | 476 | CA | ALA | A | 67 | 7520 | 7375 | 10940 | −197 | 3124 | −607 | C |
| ATOM | 477 | C | ALA | A | 67 | 106.865 | −72.663 | 83.425 | 1.00 | 71.76 | | C |
| ANISOU | 477 | C | ALA | A | 67 | 7808 | 8163 | 11294 | −193 | 2847 | −641 | C |
| ATOM | 478 | O | ALA | A | 67 | 107.817 | −71.901 | 83.546 | 1.00 | 71.29 | | O |
| ANISOU | 478 | O | ALA | A | 67 | 7738 | 8111 | 11237 | −16 | 2632 | −419 | O |
| ATOM | 479 | CB | ALA | A | 67 | 107.092 | −75.043 | 84.018 | 1.00 | 69.13 | | C |
| ANISOU | 479 | CB | ALA | A | 67 | 7768 | 7231 | 11266 | −269 | 3532 | −694 | C |
| ATOM | 480 | N | ASP | A | 68 | 106.011 | −72.632 | 82.392 | 1.00 | 69.11 | | N |
| ANISOU | 480 | N | ASP | A | 68 | 7307 | 8119 | 10832 | −377 | 2882 | −931 | N |
| ATOM | 481 | CA | ASP | A | 68 | 106.083 | −71.670 | 81.293 | 1.00 | 69.51 | | C |
| ANISOU | 481 | CA | ASP | A | 68 | 7181 | 8522 | 10708 | −315 | 2674 | −941 | C |
| ATOM | 482 | C | ASP | A | 68 | 105.819 | −70.255 | 81.814 | 1.00 | 71.31 | | C |
| ANISOU | 482 | C | ASP | A | 68 | 7400 | 8883 | 10812 | −134 | 2398 | −727 | C |
| ATOM | 483 | O | ASP | A | 68 | 106.414 | −69.321 | 81.288 | 1.00 | 72.71 | | O |
| ANISOU | 483 | O | ASP | A | 68 | 7544 | 9141 | 10943 | 13 | 2267 | −569 | O |
| ATOM | 484 | CB | ASP | A | 68 | 105.062 | −72.010 | 80.198 | 1.00 | 72.75 | | C |
| ANISOU | 484 | CB | ASP | A | 68 | 7370 | 9344 | 10930 | −507 | 2783 | −1321 | C |
| ATOM | 485 | CG | ASP | A | 68 | 105.090 | −73.458 | 79.742 | 1.00 | 95.48 | | C |
| ANISOU | 485 | CG | ASP | A | 68 | 10247 | 12082 | 13949 | −777 | 3144 | −1648 | C |
| ATOM | 486 | OD1 | ASP | A | 68 | 105.839 | −73.764 | 78.771 | 1.00 | 98.87 | | O |
| ANISOU | 486 | OD1 | ASP | A | 68 | 10630 | 12529 | 14406 | −793 | 3217 | −1703 | O |
| ATOM | 487 | OD2 | ASP | A | 68 | 104.366 | −74.296 | 80.363 | 1.00 | 100.69 | | O |
| ANISOU | 487 | OD2 | ASP | A | 68 | 10968 | 12581 | 14709 | −983 | 3403 | −1860 | O |
| ATOM | 488 | N | THR | A | 69 | 104.942 | −70.103 | 82.852 | 1.00 | 63.01 | | N |
| ANISOU | 488 | N | THR | A | 69 | 6397 | 7817 | 9726 | −150 | 2363 | −728 | N |
| ATOM | 489 | CA | THR | A | 69 | 104.618 | −68.820 | 83.477 | 1.00 | 60.46 | | C |
| ANISOU | 489 | CA | THR | A | 69 | 6090 | 7572 | 9310 | 4 | 2156 | −549 | C |
| ATOM | 490 | C | THR | A | 69 | 105.814 | −68.295 | 84.253 | 1.00 | 60.31 | | C |
| ANISOU | 490 | C | THR | A | 69 | 6192 | 7285 | 9438 | 129 | 2050 | −304 | C |
| ATOM | 491 | O | THR | A | 69 | 106.120 | −67.110 | 84.119 | 1.00 | 61.24 | | O |
| ANISOU | 491 | O | THR | A | 69 | 6298 | 7446 | 9526 | 240 | 1942 | −180 | O |
| ATOM | 492 | CB | THR | A | 69 | 103.342 | −68.914 | 84.335 | 1.00 | 60.76 | | C |
| ANISOU | 492 | CB | THR | A | 69 | 6131 | 7684 | 9270 | −68 | 2169 | −653 | C |
| ATOM | 493 | OG1 | THR | A | 69 | 102.287 | −69.437 | 83.529 | 1.00 | 61.48 | | O |
| ANISOU | 493 | OG1 | THR | A | 69 | 6039 | 8111 | 9209 | −221 | 2284 | −962 | O |
| ATOM | 494 | CG2 | THR | A | 69 | 102.914 | −67.569 | 84.925 | 1.00 | 52.65 | | C |
| ANISOU | 494 | CG2 | THR | A | 69 | 5121 | 6743 | 8140 | 93 | 1990 | −486 | C |
| ATOM | 495 | N | VAL | A | 70 | 106.494 | −69.152 | 85.049 | 1.00 | 51.77 | | N |
| ANISOU | 495 | N | VAL | A | 70 | 5213 | 5956 | 8500 | 127 | 2121 | −247 | N |
| ATOM | 496 | CA | VAL | A | 70 | 107.663 | −68.700 | 85.796 | 1.00 | 49.50 | | C |
| ANISOU | 496 | CA | VAL | A | 70 | 4960 | 5555 | 8293 | 257 | 2005 | −70 | C |
| ATOM | 497 | C | VAL | A | 70 | 108.741 | −68.271 | 84.806 | 1.00 | 53.16 | | C |
| ANISOU | 497 | C | VAL | A | 70 | 5347 | 6038 | 8815 | 284 | 1978 | −37 | C |
| ATOM | 498 | O | VAL | A | 70 | 109.236 | −67.147 | 84.895 | 1.00 | 52.40 | | O |
| ANISOU | 498 | O | VAL | A | 70 | 5213 | 5966 | 8731 | 324 | 1877 | 19 | O |
| ATOM | 499 | CB | VAL | A | 70 | 108.131 | −69.713 | 86.859 | 1.00 | 52.38 | | C |
| ANISOU | 499 | CB | VAL | A | 70 | 5422 | 5758 | 8723 | 349 | 2097 | 26 | C |
| ATOM | 500 | CG1 | VAL | A | 70 | 109.542 | −69.416 | 87.324 | 1.00 | 52.05 | | C |
| ANISOU | 500 | CG1 | VAL | A | 70 | 5321 | 5742 | 8715 | 503 | 1983 | 157 | C |
| ATOM | 501 | CG2 | VAL | A | 70 | 107.189 | −69.710 | 88.054 | 1.00 | 52.26 | | C |
| ANISOU | 501 | CG2 | VAL | A | 70 | 5488 | 5734 | 8635 | 364 | 2088 | 43 | C |
| ATOM | 502 | N | ALA | A | 71 | 109.025 | −69.132 | 83.804 | 1.00 | 51.04 | | N |
| ANISOU | 502 | N | ALA | A | 71 | 5057 | 5747 | 8589 | 234 | 2116 | −106 | N |
| ATOM | 503 | CA | ALA | A | 71 | 109.997 | −68.913 | 82.722 | 1.00 | 50.22 | | C |
| ANISOU | 503 | CA | ALA | A | 71 | 4883 | 5661 | 8536 | 253 | 2129 | −86 | C |
| ATOM | 504 | C | ALA | A | 71 | 109.687 | −67.612 | 81.972 | 1.00 | 55.43 | | C |
| ANISOU | 504 | C | ALA | A | 71 | 5487 | 6478 | 9095 | 284 | 2064 | −64 | C |
| ATOM | 505 | O | ALA | A | 71 | 110.592 | −66.792 | 81.809 | 1.00 | 57.32 | | O |
| ANISOU | 505 | O | ALA | A | 71 | 5710 | 6659 | 9409 | 333 | 2042 | 22 | O |
| ATOM | 506 | CB | ALA | A | 71 | 109.999 | −70.098 | 81.773 | 1.00 | 50.43 | | C |
| ANISOU | 506 | CB | ALA | A | 71 | 4900 | 5668 | 8592 | 167 | 2319 | −215 | C |
| ATOM | 507 | N | ALA | A | 72 | 108.397 | −67.380 | 81.614 | 1.00 | 50.10 | | N |
| ANISOU | 507 | N | ALA | A | 72 | 4780 | 6011 | 8243 | 279 | 2069 | −137 | N |
| ATOM | 508 | CA | ALA | A | 72 | 107.925 | −66.162 | 80.954 | 1.00 | 49.11 | | C |
| ANISOU | 508 | CA | ALA | A | 72 | 4621 | 6072 | 7968 | 410 | 2059 | −54 | C |
| ATOM | 509 | C | ALA | A | 72 | 108.140 | −64.925 | 81.827 | 1.00 | 50.91 | | C |
| ANISOU | 509 | C | ALA | A | 72 | 4938 | 6133 | 8273 | 479 | 2018 | 87 | C |
| ATOM | 510 | O | ALA | A | 72 | 108.316 | −63.856 | 81.279 | 1.00 | 51.36 | | O |
| ANISOU | 510 | O | ALA | A | 72 | 5021 | 6182 | 8310 | 600 | 2106 | 208 | O |
| ATOM | 511 | CB | ALA | A | 72 | 106.467 | −66.293 | 80.580 | 1.00 | 50.29 | | C |
| ANISOU | 511 | CB | ALA | A | 72 | 4673 | 6567 | 7869 | 429 | 2062 | −174 | C |
| ATOM | 512 | N | GLY | A | 73 | 108.150 | −65.091 | 83.152 | 1.00 | 45.64 | | N |
| ANISOU | 512 | N | GLY | A | 73 | 4320 | 5331 | 7692 | 407 | 1933 | 63 | N |
| ATOM | 513 | CA | GLY | A | 73 | 108.444 | −64.041 | 84.122 | 1.00 | 45.04 | | C |
| ANISOU | 513 | CA | GLY | A | 73 | 4296 | 5118 | 7698 | 413 | 1903 | 112 | C |
| ATOM | 514 | C | GLY | A | 73 | 109.915 | −63.652 | 84.055 | 1.00 | 50.36 | | C |
| ANISOU | 514 | C | GLY | A | 73 | 4935 | 5654 | 8546 | 367 | 1945 | 108 | C |
| ATOM | 515 | O | GLY | A | 73 | 110.253 | −62.482 | 84.227 | 1.00 | 50.39 | | O |
| ANISOU | 515 | O | GLY | A | 73 | 4964 | 5548 | 8633 | 349 | 2038 | 109 | O |
| ATOM | 516 | N | LEU | A | 74 | 110.810 | −64.607 | 83.767 | 1.00 | 46.93 | | N |
| ANISOU | 516 | N | LEU | A | 74 | 4436 | 5217 | 8177 | 339 | 1924 | 82 | N |
| ATOM | 517 | CA | LEU | A | 74 | 112.236 | −64.298 | 83.609 | 1.00 | 46.92 | | C |
| ANISOU | 517 | CA | LEU | A | 74 | 4354 | 5149 | 8324 | 296 | 1964 | 51 | C |
| ATOM | 518 | C | LEU | A | 74 | 112.495 | −63.604 | 82.242 | 1.00 | 54.81 | | C |
| ANISOU | 518 | C | LEU | A | 74 | 5378 | 6089 | 9360 | 323 | 2139 | 111 | C |
| ATOM | 519 | O | LEU | A | 74 | 113.404 | −62.758 | 82.104 | 1.00 | 54.54 | | O |
| ANISOU | 519 | O | LEU | A | 74 | 5315 | 5940 | 9467 | 261 | 2264 | 75 | O |
| ATOM | 520 | CB | LEU | A | 74 | 113.097 | −65.569 | 83.716 | 1.00 | 46.30 | | C |
| ANISOU | 520 | CB | LEU | A | 74 | 4198 | 5110 | 8282 | 319 | 1910 | 41 | C |
| ATOM | 521 | CG | LEU | A | 74 | 113.056 | −66.326 | 85.040 | 1.00 | 50.04 | | C |
| ANISOU | 521 | CG | LEU | A | 74 | 4659 | 5652 | 8702 | 382 | 1802 | 47 | C |
| ATOM | 522 | CD1 | LEU | A | 74 | 113.272 | −67.784 | 84.820 | 1.00 | 50.41 | | C |
| ANISOU | 522 | CD1 | LEU | A | 74 | 4736 | 5665 | 8751 | 473 | 1868 | 114 | C |
| ATOM | 523 | CD2 | LEU | A | 74 | 114.088 | −65.811 | 85.998 | 1.00 | 50.91 | | C |
| ANISOU | 523 | CD2 | LEU | A | 74 | 4612 | 5896 | 8835 | 383 | 1714 | −34 | C |
| ATOM | 524 | N | LEU | A | 75 | 111.668 | −63.934 | 81.239 | 1.00 | 53.53 | | N |
| ANISOU | 524 | N | LEU | A | 75 | 5252 | 6034 | 9051 | 417 | 2179 | 180 | N |
| ATOM | 525 | CA | LEU | A | 75 | 111.856 | −63.393 | 79.894 | 1.00 | 54.10 | | C |
| ANISOU | 525 | CA | LEU | A | 75 | 5342 | 6125 | 9090 | 517 | 2350 | 276 | C |
| ATOM | 526 | C | LEU | A | 75 | 110.937 | −62.217 | 79.496 | 1.00 | 61.44 | | C |
| ANISOU | 526 | C | LEU | A | 75 | 6366 | 7095 | 9883 | 699 | 2501 | 426 | C |
| ATOM | 527 | O | LEU | A | 75 | 111.167 | −61.656 | 78.426 | 1.00 | 61.56 | | O |
| ANISOU | 527 | O | LEU | A | 75 | 6418 | 7114 | 9857 | 845 | 2697 | 558 | O |
| ATOM | 528 | CB | LEU | A | 75 | 111.717 | −64.530 | 78.842 | 1.00 | 53.47 | | C |
| ANISOU | 528 | CB | LEU | A | 75 | 5192 | 6234 | 8889 | 541 | 2331 | 237 | C |
| ATOM | 529 | CG | LEU | A | 75 | 112.598 | −65.783 | 78.830 | 1.00 | 56.42 | | C |
| ANISOU | 529 | CG | LEU | A | 75 | 5503 | 6552 | 9381 | 431 | 2287 | 137 | C |
| ATOM | 530 | CD1 | LEU | A | 75 | 111.802 | −67.039 | 78.886 | 1.00 | 55.40 | | C |
| ANISOU | 530 | CD1 | LEU | A | 75 | 5355 | 6536 | 9159 | 372 | 2251 | 15 | C |
| ATOM | 531 | CD2 | LEU | A | 75 | 113.919 | −65.669 | 79.572 | 1.00 | 58.33 | | C |
| ANISOU | 531 | CD2 | LEU | A | 75 | 5710 | 6618 | 9833 | 366 | 2260 | 128 | C |
| ATOM | 532 | N | HIS | A | 76 | 109.937 | −61.834 | 80.335 | 1.00 | 59.26 | | N |
| ANISOU | 532 | N | HIS | A | 76 | 6140 | 6850 | 9529 | 732 | 2445 | 434 | N |
| ATOM | 533 | CA | HIS | A | 76 | 108.917 | −60.810 | 80.051 | 1.00 | 60.00 | | C |
| ANISOU | 533 | CA | HIS | A | 76 | 6320 | 7021 | 9456 | 968 | 2596 | 605 | C |
| ATOM | 534 | C | HIS | A | 76 | 109.429 | −59.490 | 79.512 | 1.00 | 68.77 | | C |
| ANISOU | 534 | C | HIS | A | 76 | 7572 | 7890 | 10668 | 1112 | 2940 | 786 | C |
| ATOM | 535 | O | HIS | A | 76 | 108.609 | −58.751 | 78.962 | 1.00 | 71.05 | | O |
| ANISOU | 535 | O | HIS | A | 76 | 7942 | 8290 | 10766 | 1418 | 3125 | 1003 | O |
| ATOM | 536 | CB | HIS | A | 76 | 107.989 | −60.543 | 81.255 | 1.00 | 60.51 | | C |
| ANISOU | 536 | CB | HIS | A | 76 | 6425 | 7077 | 9488 | 941 | 2501 | 565 | C |
| ATOM | 537 | CG | HIS | A | 76 | 108.549 | −59.663 | 82.336 | 1.00 | 63.56 | | C |
| ANISOU | 537 | CG | HIS | A | 76 | 6905 | 7131 | 10112 | 798 | 2596 | 509 | C |
| ATOM | 538 | ND1 | HIS | A | 76 | 109.396 | −60.165 | 83.308 | 1.00 | 65.10 | | N |
| ANISOU | 538 | ND1 | HIS | A | 76 | 7025 | 7236 | 10475 | 548 | 2440 | 312 | N |
| ATOM | 539 | CD2 | HIS | A | 76 | 108.301 | −58.357 | 82.605 | 1.00 | 64.80 | | C |
| ANISOU | 539 | CD2 | HIS | A | 76 | 7206 | 7079 | 10337 | 882 | 2855 | 599 | C |
| ATOM | 540 | CE1 | HIS | A | 76 | 109.658 | −59.154 | 84.119 | 1.00 | 64.37 | | C |
| ANISOU | 540 | CE1 | HIS | A | 76 | 6991 | 6937 | 10531 | 448 | 2576 | 234 | C |
| ATOM | 541 | NE2 | HIS | A | 76 | 109.032 | −58.040 | 83.728 | 1.00 | 64.48 | | N |
| ANISOU | 541 | NE2 | HIS | A | 76 | 7158 | 6819 | 10523 | 617 | 2855 | 392 | N |
| ATOM | 542 | N | ASP | A | 77 | 110.747 | −59.182 | 79.644 | 1.00 | 65.69 | | N |
| ANISOU | 542 | N | ASP | A | 77 | 7205 | 7192 | 10563 | 918 | 3073 | 700 | N |
| ATOM | 543 | CA | ASP | A | 77 | 111.309 | −57.927 | 79.139 | 1.00 | 65.21 | | C |
| ANISOU | 543 | CA | ASP | A | 77 | 7297 | 6821 | 10657 | 1000 | 3501 | 831 | C |
| ATOM | 544 | C | ASP | A | 77 | 112.297 | −58.129 | 77.997 | 1.00 | 70.83 | | C |
| ANISOU | 544 | C | ASP | A | 77 | 7981 | 7503 | 11426 | 1018 | 3637 | 880 | C |
| ATOM | 545 | O | ASP | A | 77 | 112.732 | −57.138 | 77.391 | 1.00 | 71.79 | | O |
| ANISOU | 545 | O | ASP | A | 77 | 8252 | 7360 | 11663 | 1124 | 4057 | 1025 | O |
| ATOM | 546 | CB | ASP | A | 77 | 111.944 | −57.113 | 80.280 | 1.00 | 66.86 | | C |
| ANISOU | 546 | CB | ASP | A | 77 | 7551 | 6679 | 11173 | 718 | 3659 | 625 | C |
| ATOM | 547 | CG | ASP | A | 77 | 110.967 | −56.498 | 81.253 | 1.00 | 86.48 | | C |
| ANISOU | 547 | CG | ASP | A | 77 | 10130 | 9106 | 13622 | 750 | 3682 | 626 | C |
| ATOM | 548 | OD1 | ASP | A | 77 | 109.956 | −55.901 | 80.794 | 1.00 | 90.04 | | O |
| ANISOU | 548 | OD1 | ASP | A | 77 | 10731 | 9578 | 13902 | 1077 | 3873 | 896 | O |
| ATOM | 549 | OD2 | ASP | A | 77 | 111.221 | −56.580 | 82.473 | 1.00 | 94.53 | | O |
| ANISOU | 549 | OD2 | ASP | A | 77 | 11064 | 10092 | 14763 | 479 | 3525 | 365 | O |
| ATOM | 550 | N | THR | A | 78 | 112.647 | −59.404 | 77.671 | 1.00 | 67.20 | | N |
| ANISOU | 550 | N | THR | A | 78 | 7354 | 7282 | 10898 | 928 | 3341 | 769 | N |
| ATOM | 551 | CA | THR | A | 78 | 113.633 | −59.728 | 76.616 | 1.00 | 66.43 | | C |
| ANISOU | 551 | CA | THR | A | 78 | 7210 | 7176 | 10856 | 924 | 3439 | 788 | C |
| ATOM | 552 | C | THR | A | 78 | 113.176 | −59.346 | 75.202 | 1.00 | 71.43 | | C |
| ANISOU | 552 | C | THR | A | 78 | 7924 | 7969 | 11246 | 1284 | 3670 | 1067 | C |
| ATOM | 553 | O | THR | A | 78 | 113.834 | −59.712 | 74.247 | 1.00 | 68.39 | | O |
| ANISOU | 553 | O | THR | A | 78 | 7493 | 7644 | 10847 | 1314 | 3729 | 1092 | O |
| ATOM | 554 | CB | THR | A | 78 | 114.084 | −61.197 | 76.662 | 1.00 | 65.56 | | C |
| ANISOU | 554 | CB | THR | A | 78 | 6920 | 7251 | 10739 | 759 | 3116 | 607 | C |
| ATOM | 555 | OG1 | THR | A | 78 | 112.959 | −62.038 | 76.450 | 1.00 | 68.08 | | O |
| ANISOU | 555 | OG1 | THR | A | 78 | 7183 | 7899 | 10785 | 866 | 2911 | 609 | O |
| ATOM | 556 | CG2 | THR | A | 78 | 114.812 | −61.572 | 77.943 | 1.00 | 59.57 | | C |
| ANISOU | 556 | CG2 | THR | A | 78 | 6062 | 6380 | 10190 | 492 | 2939 | 385 | C |
| ATOM | 557 | N | LEU | A | 79 | 112.071 | −58.611 | 75.057 | 1.00 | 72.71 | | N |
| ANISOU | 557 | N | LEU | A | 79 | 8196 | 8242 | 11189 | 1596 | 3813 | 1289 | N |
| ATOM | 558 | CA | LEU | A | 79 | 111.631 | −58.207 | 73.732 | 1.00 | 74.72 | | C |
| ANISOU | 558 | CA | LEU | A | 79 | 8504 | 8746 | 11141 | 2031 | 4050 | 1594 | C |
| ATOM | 559 | C | LEU | A | 79 | 111.632 | −56.681 | 73.536 | 1.00 | 85.51 | | C |
| ANISOU | 559 | C | LEU | A | 79 | 10145 | 9758 | 12587 | 2319 | 4594 | 1913 | C |
| ATOM | 560 | O | LEU | A | 79 | 111.901 | −56.216 | 72.420 | 1.00 | 84.58 | | O |
| ANISOU | 560 | O | LEU | A | 79 | 10129 | 9646 | 12361 | 2632 | 4934 | 2182 | O |
| ATOM | 561 | CB | LEU | A | 79 | 110.272 | −58.830 | 73.363 | 1.00 | 74.28 | | C |
| ANISOU | 561 | CB | LEU | A | 79 | 8279 | 9318 | 10626 | 2280 | 3784 | 1615 | C |
| ATOM | 562 | CG | LEU | A | 79 | 110.209 | −60.352 | 73.144 | 1.00 | 78.31 | | C |
| ANISOU | 562 | CG | LEU | A | 79 | 8535 | 10204 | 11017 | 2051 | 3401 | 1311 | C |
| ATOM | 563 | CD1 | LEU | A | 79 | 108.891 | −60.727 | 72.584 | 1.00 | 78.08 | | C |
| ANISOU | 563 | CD1 | LEU | A | 79 | 8314 | 10838 | 10516 | 2307 | 3263 | 1297 | C |
| ATOM | 564 | CD2 | LEU | A | 79 | 111.254 | −60.853 | 72.162 | 1.00 | 82.35 | | C |
| ANISOU | 564 | CD2 | LEU | A | 79 | 8996 | 10709 | 11584 | 1989 | 3461 | 1273 | C |
| ATOM | 565 | N | GLU | A | 80 | 111.341 | −55.909 | 74.606 | 1.00 | 88.51 | | N |
| ANISOU | 565 | N | GLU | A | 80 | 10662 | 9810 | 13158 | 2225 | 4727 | 1887 | N |
| ATOM | 566 | CA | GLU | A | 80 | 111.298 | −54.446 | 74.526 | 1.00 | 91.65 | | C |
| ANISOU | 566 | CA | GLU | A | 80 | 11359 | 9781 | 13682 | 2473 | 5337 | 2167 | C |
| ATOM | 567 | C | GLU | A | 80 | 112.690 | −53.898 | 74.343 | 1.00 | 100.80 | | C |
| ANISOU | 567 | C | GLU | A | 80 | 12644 | 10405 | 15252 | 2216 | 5755 | 2079 | C |
| ATOM | 568 | O | GLU | A | 80 | 113.552 | −54.100 | 75.207 | 1.00 | 99.83 | | O |
| ANISOU | 568 | O | GLU | A | 80 | 12420 | 10042 | 15470 | 1707 | 5631 | 1694 | O |
| ATOM | 569 | CB | GLU | A | 80 | 110.514 | −53.759 | 75.681 | 1.00 | 93.65 | | C |
| ANISOU | 569 | CB | GLU | A | 80 | 11729 | 9840 | 14016 | 2451 | 5410 | 2146 | C |
| ATOM | 570 | CG | GLU | A | 80 | 110.934 | −54.117 | 77.105 | 1.00 | 111.63 | | C |
| ANISOU | 570 | CG | GLU | A | 80 | 13888 | 11931 | 16595 | 1889 | 5107 | 1701 | C |
| ATOM | 571 | CD | GLU | A | 80 | 110.760 | −53.033 | 78.160 | 1.00 | 144.33 | | C |
| ANISOU | 571 | CD | GLU | A | 80 | 18213 | 15634 | 20992 | 1758 | 5436 | 1623 | C |
| ATOM | 572 | OE1 | GLU | A | 80 | 109.601 | −52.663 | 78.463 | 1.00 | 138.45 | | O |
| ANISOU | 572 | OE1 | GLU | A | 80 | 17557 | 14995 | 20055 | 2037 | 5458 | 1806 | O |
| ATOM | 573 | OE2 | GLU | A | 80 | 111.791 | −52.569 | 78.701 | 1.00 | 145.26 | | O |
| ANISOU | 573 | OE2 | GLU | A | 80 | 18358 | 15338 | 21498 | 1355 | 5678 | 1335 | O |
| ATOM | 574 | N | ASP | A | 81 | 112.917 | −53.321 | 73.122 | 1.00 | 101.97 | | N |
| ANISOU | 574 | N | ASP | A | 81 | 12968 | 10465 | 15313 | 2599 | 6230 | 2433 | N |
| ATOM | 575 | CA | ASP | A | 81 | 114.138 | −52.707 | 72.564 | 1.00 | 102.99 | | C |
| ANISOU | 575 | CA | ASP | A | 81 | 13255 | 10105 | 15769 | 2484 | 6769 | 2454 | C |
| ATOM | 576 | C | ASP | A | 81 | 115.367 | −53.595 | 72.752 | 1.00 | 105.62 | | C |
| ANISOU | 576 | C | ASP | A | 81 | 13339 | 10440 | 16352 | 1949 | 6434 | 2027 | C |
| ATOM | 577 | O | ASP | A | 81 | 116.494 | −53.114 | 72.737 | 1.00 | 103.99 | | O |
| ANISOU | 577 | O | ASP | A | 81 | 13192 | 9801 | 16520 | 1657 | 6814 | 1862 | O |
| ATOM | 578 | CB | ASP | A | 81 | 114.348 | −51.273 | 73.111 | 1.00 | 106.18 | | C |
| ANISOU | 578 | CB | ASP | A | 81 | 13979 | 9803 | 16563 | 2406 | 7492 | 2483 | C |
| ATOM | 579 | CG | ASP | A | 81 | 113.618 | −50.170 | 72.350 | 1.00 | 119.93 | | C |
| ANISOU | 579 | CG | ASP | A | 81 | 16082 | 11366 | 18121 | 3076 | 8168 | 3063 | C |
| ATOM | 580 | OD2 | ASP | A | 81 | 112.537 | −49.719 | 72.838 | 1.00 | 120.55 | | O |
| ANISOU | 580 | OD2 | ASP | A | 81 | 16268 | 11493 | 18040 | 3355 | 8216 | 3244 | O |
| ATOM | 581 | OD1 | ASP | A | 81 | 114.143 | −49.732 | 71.277 | 1.00 | 121.08 | | O |
| ANISOU | 581 | OD1 | ASP | A | 81 | 16414 | 11313 | 18278 | 3353 | 8690 | 3358 | O |
| ATOM | 582 | N | CYS | A | 82 | 115.122 | −54.894 | 72.959 | 1.00 | 103.95 | | N |
| ANISOU | 582 | N | CYS | A | 82 | 12844 | 10719 | 15932 | 1827 | 5760 | 1839 | N |
| ATOM | 583 | CA | CYS | A | 82 | 116.125 | −55.930 | 73.157 | 1.00 | 104.96 | | C |
| ANISOU | 583 | CA | CYS | A | 82 | 12721 | 10944 | 16217 | 1419 | 5385 | 1489 | C |
| ATOM | 584 | C | CYS | A | 82 | 116.324 | −56.634 | 71.827 | 1.00 | 108.73 | | C |
| ANISOU | 584 | C | CYS | A | 82 | 13124 | 11735 | 16453 | 1645 | 5307 | 1647 | C |
| ATOM | 585 | O | CYS | A | 82 | 115.363 | −56.810 | 71.060 | 1.00 | 107.97 | | O |
| ANISOU | 585 | O | CYS | A | 82 | 13039 | 12032 | 15952 | 2059 | 5242 | 1910 | O |
| ATOM | 586 | CB | CYS | A | 82 | 115.694 | −56.915 | 74.245 | 1.00 | 105.97 | | C |
| ANISOU | 586 | CB | CYS | A | 82 | 12634 | 11352 | 16278 | 1187 | 4797 | 1220 | C |
| ATOM | 587 | SG | CYS | A | 82 | 116.222 | −56.479 | 75.926 | 1.00 | 110.17 | | S |
| ANISOU | 587 | SG | CYS | A | 82 | 13103 | 11579 | 17175 | 718 | 4775 | 832 | S |
| ATOM | 588 | N | GLY | A | 83 | 117.570 | −57.045 | 71.583 | 1.00 | 104.98 | | N |
| ANISOU | 588 | N | GLY | A | 83 | 12539 | 11142 | 16205 | 1370 | 5306 | 1453 | N |
| ATOM | 589 | CA | GLY | A | 83 | 117.980 | −57.759 | 70.380 | 1.00 | 104.27 | | C |
| ANISOU | 589 | CA | GLY | A | 83 | 12366 | 11302 | 15950 | 1505 | 5245 | 1537 | C |
| ATOM | 590 | C | GLY | A | 83 | 117.532 | −59.212 | 70.346 | 1.00 | 105.77 | | C |
| ANISOU | 590 | C | GLY | A | 83 | 12325 | 11977 | 15886 | 1471 | 4687 | 1390 | C |
| ATOM | 591 | O | GLY | A | 83 | 118.233 | −60.055 | 69.764 | 1.00 | 106.14 | | O |
| ANISOU | 591 | O | GLY | A | 83 | 12241 | 12146 | 15942 | 1375 | 4563 | 1283 | O |
| ATOM | 592 | N | VAL | A | 84 | 116.356 | −59.516 | 70.966 | 1.00 | 97.83 | | N |
| ANISOU | 592 | N | VAL | A | 84 | 11275 | 11225 | 14670 | 1537 | 4397 | 1367 | N |
| ATOM | 593 | CA | VAL | A | 84 | 115.769 | −60.853 | 70.987 | 1.00 | 96.10 | | C |
| ANISOU | 593 | CA | VAL | A | 84 | 10861 | 11427 | 14227 | 1482 | 3960 | 1197 | C |
| ATOM | 594 | C | VAL | A | 84 | 114.533 | −60.837 | 70.118 | 1.00 | 97.99 | | C |
| ANISOU | 594 | C | VAL | A | 84 | 11074 | 12156 | 14002 | 1870 | 3957 | 1378 | C |
| ATOM | 595 | O | VAL | A | 84 | 113.697 | −59.933 | 70.232 | 1.00 | 97.41 | | O |
| ANISOU | 595 | O | VAL | A | 84 | 11115 | 12125 | 13774 | 2148 | 4114 | 1598 | O |
| ATOM | 596 | CB | VAL | A | 84 | 115.493 | −61.376 | 72.414 | 1.00 | 99.88 | | C |
| ANISOU | 596 | CB | VAL | A | 84 | 11265 | 11846 | 14839 | 1206 | 3643 | 963 | C |
| ATOM | 597 | CG1 | VAL | A | 84 | 114.789 | −62.735 | 72.401 | 1.00 | 99.87 | | C |
| ANISOU | 597 | CG1 | VAL | A | 84 | 11105 | 12216 | 14626 | 1154 | 3308 | 793 | C |
| ATOM | 598 | CG2 | VAL | A | 84 | 116.773 | −61.422 | 73.244 | 1.00 | 99.60 | | C |
| ANISOU | 598 | CG2 | VAL | A | 84 | 11185 | 11476 | 15183 | 878 | 3630 | 769 | C |
| ATOM | 599 | N | ALA | A | 85 | 114.464 | −61.804 | 69.200 | 1.00 | 93.92 | | N |
| ANISOU | 599 | N | ALA | A | 85 | 10391 | 12045 | 13250 | 1906 | 3814 | 1275 | N |
| ATOM | 600 | CA | ALA | A | 85 | 113.366 | −61.943 | 68.261 | 1.00 | 93.72 | | C |
| ANISOU | 600 | CA | ALA | A | 85 | 10239 | 12651 | 12718 | 2252 | 3781 | 1353 | C |
| ATOM | 601 | C | ALA | A | 85 | 112.336 | −62.874 | 68.862 | 1.00 | 96.71 | | C |
| ANISOU | 601 | C | ALA | A | 85 | 10431 | 13368 | 12947 | 2077 | 3441 | 1058 | C |
| ATOM | 602 | O | ALA | A | 85 | 112.741 | −63.870 | 69.462 | 1.00 | 96.30 | | O |
| ANISOU | 602 | O | ALA | A | 85 | 10321 | 13130 | 13140 | 1697 | 3252 | 775 | O |
| ATOM | 603 | CB | ALA | A | 85 | 113.879 | −62.509 | 66.947 | 1.00 | 94.51 | | C |
| ANISOU | 603 | CB | ALA | A | 85 | 10225 | 13043 | 12643 | 2330 | 3837 | 1316 | C |
| ATOM | 604 | N | PRO | A | 86 | 111.013 | −62.611 | 68.694 | 1.00 | 93.04 | | N |
| ANISOU | 604 | N | PRO | A | 86 | 9861 | 13419 | 12071 | 2364 | 3393 | 1116 | N |
| ATOM | 605 | CA | PRO | A | 86 | 110.001 | −63.516 | 69.275 | 1.00 | 93.25 | | C |
| ANISOU | 605 | CA | PRO | A | 86 | 9689 | 13770 | 11972 | 2149 | 3109 | 781 | C |
| ATOM | 606 | C | PRO | A | 86 | 110.080 | −64.947 | 68.763 | 1.00 | 98.13 | | C |
| ANISOU | 606 | C | PRO | A | 86 | 10081 | 14674 | 12529 | 1855 | 2973 | 372 | C |
| ATOM | 607 | O | PRO | A | 86 | 109.660 | −65.875 | 69.450 | 1.00 | 98.11 | | O |
| ANISOU | 607 | O | PRO | A | 86 | 9992 | 14658 | 12629 | 1530 | 2819 | 50 | O |
| ATOM | 608 | CB | PRO | A | 86 | 108.666 | −62.856 | 68.906 | 1.00 | 95.00 | | C |
| ANISOU | 608 | CB | PRO | A | 86 | 9791 | 14609 | 11697 | 2587 | 3132 | 936 | C |
| ATOM | 609 | CG | PRO | A | 86 | 108.971 | −61.963 | 67.777 | 1.00 | 99.41 | | C |
| ANISOU | 609 | CG | PRO | A | 86 | 10422 | 15338 | 12011 | 3071 | 3413 | 1316 | C |
| ATOM | 610 | CD | PRO | A | 86 | 110.373 | −61.476 | 68.003 | 1.00 | 94.78 | | C |
| ANISOU | 610 | CD | PRO | A | 86 | 10133 | 13962 | 11918 | 2924 | 3631 | 1498 | C |
| ATOM | 611 | N | GLU | A | 87 | 110.647 | −65.121 | 67.572 | 1.00 | 95.87 | | N |
| ANISOU | 611 | N | GLU | A | 87 | 9724 | 14602 | 12103 | 1965 | 3083 | 384 | N |
| ATOM | 612 | CA | GLU | A | 87 | 110.837 | −66.423 | 66.929 | 1.00 | 96.48 | | C |
| ANISOU | 612 | CA | GLU | A | 87 | 9599 | 14928 | 12130 | 1692 | 3030 | −13 | C |
| ATOM | 613 | C | GLU | A | 87 | 111.815 | −67.327 | 67.730 | 1.00 | 98.07 | | C |
| ANISOU | 613 | C | GLU | A | 87 | 9933 | 14478 | 12853 | 1255 | 2998 | −186 | C |
| ATOM | 614 | O | GLU | A | 87 | 111.597 | −68.544 | 67.818 | 1.00 | 97.77 | | O |
| ANISOU | 614 | O | GLU | A | 87 | 9774 | 14509 | 12865 | 944 | 2961 | −567 | O |
| ATOM | 615 | CB | GLU | A | 87 | 111.288 | −66.250 | 65.451 | 1.00 | 98.45 | | C |
| ANISOU | 615 | CB | GLU | A | 87 | 9759 | 15554 | 12092 | 1962 | 3179 | 89 | C |
| ATOM | 616 | CG | GLU | A | 87 | 110.441 | −65.321 | 64.569 | 1.00 | 112.33 | | C |
| ANISOU | 616 | CG | GLU | A | 87 | 11391 | 18016 | 13273 | 2522 | 3259 | 346 | C |
| ATOM | 617 | CD | GLU | A | 87 | 108.923 | −65.429 | 64.575 | 1.00 | 142.40 | | C |
| ANISOU | 617 | CD | GLU | A | 87 | 14900 | 22605 | 16602 | 2658 | 3111 | 141 | C |
| ATOM | 618 | OE1 | GLU | A | 87 | 108.391 | −66.563 | 64.617 | 1.00 | 141.82 | | O |
| ANISOU | 618 | OE1 | GLU | A | 87 | 14562 | 22865 | 16459 | 2287 | 2970 | −388 | O |
| ATOM | 619 | OE2 | GLU | A | 87 | 108.264 | −64.366 | 64.510 | 1.00 | 141.75 | | O |
| ANISOU | 619 | OE2 | GLU | A | 87 | 14841 | 22810 | 16209 | 3148 | 3180 | 502 | O |
| ATOM | 620 | N | GLU | A | 88 | 112.864 | −66.710 | 68.334 | 1.00 | 91.68 | | N |
| ANISOU | 620 | N | GLU | A | 88 | 9358 | 13063 | 12416 | 1251 | 3054 | 85 | N |
| ATOM | 621 | CA | GLU | A | 88 | 113.857 | −67.379 | 69.174 | 1.00 | 89.96 | | C |
| ANISOU | 621 | CA | GLU | A | 88 | 9240 | 12302 | 12637 | 947 | 3020 | −4 | C |
| ATOM | 622 | C | GLU | A | 88 | 113.189 | −67.940 | 70.451 | 1.00 | 90.23 | | C |
| ANISOU | 622 | C | GLU | A | 88 | 9288 | 12206 | 12789 | 741 | 2878 | −167 | C |
| ATOM | 623 | O | GLU | A | 88 | 113.463 | −69.082 | 70.815 | 1.00 | 90.74 | | O |
| ANISOU | 623 | O | GLU | A | 88 | 9345 | 12096 | 13037 | 506 | 2879 | −376 | O |
| ATOM | 624 | CB | GLU | A | 88 | 114.995 | −66.409 | 69.528 | 1.00 | 91.40 | | C |
| ANISOU | 624 | CB | GLU | A | 88 | 9594 | 12023 | 13110 | 1009 | 3116 | 273 | C |
| ATOM | 625 | CG | GLU | A | 88 | 116.185 | −67.092 | 70.185 | 1.00 | 107.13 | | C |
| ANISOU | 625 | CG | GLU | A | 88 | 11617 | 13610 | 15479 | 767 | 3084 | 182 | C |
| ATOM | 626 | CD | GLU | A | 88 | 117.459 | −66.277 | 70.327 | 1.00 | 142.71 | | C |
| ANISOU | 626 | CD | GLU | A | 88 | 16199 | 17772 | 20252 | 771 | 3203 | 342 | C |
| ATOM | 627 | OE1 | GLU | A | 88 | 118.549 | −66.896 | 70.291 | 1.00 | 146.90 | | O |
| ANISOU | 627 | OE1 | GLU | A | 88 | 16686 | 18141 | 20988 | 651 | 3218 | 266 | O |
| ATOM | 628 | OE2 | GLU | A | 88 | 117.378 | −65.034 | 70.479 | 1.00 | 139.18 | | O |
| ANISOU | 628 | OE2 | GLU | A | 88 | 15848 | 17216 | 19818 | 887 | 3319 | 521 | O |
| ATOM | 629 | N | LEU | A | 89 | 112.296 | −67.160 | 71.102 | 1.00 | 82.30 | | N |
| ANISOU | 629 | N | LEU | A | 89 | 8318 | 11279 | 11674 | 857 | 2802 | −56 | N |
| ATOM | 630 | CA | LEU | A | 89 | 111.594 | −67.592 | 72.313 | 1.00 | 80.20 | | C |
| ANISOU | 630 | CA | LEU | A | 89 | 8071 | 10909 | 11494 | 689 | 2682 | −187 | C |
| ATOM | 631 | C | LEU | A | 89 | 110.696 | −68.779 | 72.024 | 1.00 | 86.68 | | C |
| ANISOU | 631 | C | LEU | A | 89 | 8724 | 12060 | 12150 | 504 | 2685 | −553 | C |
| ATOM | 632 | O | LEU | A | 89 | 110.636 | −69.684 | 72.853 | 1.00 | 86.28 | | O |
| ANISOU | 632 | O | LEU | A | 89 | 8722 | 11761 | 12300 | 275 | 2694 | −718 | O |
| ATOM | 633 | CB | LEU | A | 89 | 110.822 | −66.443 | 73.002 | 1.00 | 78.69 | | C |
| ANISOU | 633 | CB | LEU | A | 89 | 7949 | 10742 | 11209 | 863 | 2623 | 7 | C |
| ATOM | 634 | CG | LEU | A | 89 | 111.665 | −65.283 | 73.553 | 1.00 | 80.48 | | C |
| ANISOU | 634 | CG | LEU | A | 89 | 8354 | 10554 | 11671 | 951 | 2688 | 282 | C |
| ATOM | 635 | CD1 | LEU | A | 89 | 110.829 | −64.115 | 73.832 | 1.00 | 79.71 | | C |
| ANISOU | 635 | CD1 | LEU | A | 89 | 8326 | 10535 | 11427 | 1173 | 2730 | 472 | C |
| ATOM | 636 | CD2 | LEU | A | 89 | 112.378 | −65.654 | 74.823 | 1.00 | 80.32 | | C |
| ANISOU | 636 | CD2 | LEU | A | 89 | 8406 | 10134 | 11980 | 725 | 2598 | 218 | C |
| ATOM | 637 | N | GLU | A | 90 | 110.065 | −68.818 | 70.821 | 1.00 | 85.61 | | N |
| ANISOU | 637 | N | GLU | A | 90 | 8381 | 12498 | 11649 | 604 | 2724 | −701 | N |
| ATOM | 638 | CA | GLU | A | 90 | 109.195 | −69.931 | 70.396 | 1.00 | 86.78 | | C |
| ANISOU | 638 | CA | GLU | A | 90 | 8300 | 13062 | 11611 | 371 | 2775 | −1165 | C |
| ATOM | 639 | C | GLU | A | 90 | 110.022 | −71.184 | 70.114 | 1.00 | 90.90 | | C |
| ANISOU | 639 | C | GLU | A | 90 | 8850 | 13297 | 12388 | 85 | 2939 | −1408 | C |
| ATOM | 640 | O | GLU | A | 90 | 109.585 | −72.303 | 70.407 | 1.00 | 91.18 | | O |
| ANISOU | 640 | O | GLU | A | 90 | 8842 | 13276 | 12526 | −224 | 3070 | −1773 | O |
| ATOM | 641 | CB | GLU | A | 90 | 108.338 | −69.549 | 69.166 | 1.00 | 88.49 | | C |
| ANISOU | 641 | CB | GLU | A | 90 | 8221 | 14107 | 11295 | 594 | 2759 | −1283 | C |
| ATOM | 642 | CG | GLU | A | 90 | 107.179 | −70.502 | 68.881 | 1.00 | 99.82 | | C |
| ANISOU | 642 | CG | GLU | A | 90 | 9337 | 16114 | 12477 | 331 | 2797 | −1845 | C |
| ATOM | 643 | CD | GLU | A | 90 | 106.103 | −69.914 | 67.989 | 1.00 | 117.78 | | C |
| ANISOU | 643 | CD | GLU | A | 90 | 11262 | 19345 | 14142 | 631 | 2717 | −1932 | C |
| ATOM | 644 | OE1 | GLU | A | 90 | 106.371 | −69.740 | 66.779 | 1.00 | 108.02 | | O |
| ANISOU | 644 | OE1 | GLU | A | 90 | 9868 | 18578 | 12597 | 848 | 2753 | −1918 | O |
| ATOM | 645 | OE2 | GLU | A | 90 | 105.004 | −69.601 | 68.504 | 1.00 | 109.88 | | O |
| ANISOU | 645 | OE2 | GLU | A | 90 | 10138 | 18665 | 12948 | 694 | 2623 | −1990 | O |
| ATOM | 646 | N | ARG | A | 91 | 111.215 | −70.979 | 69.541 | 1.00 | 86.29 | | N |
| ANISOU | 646 | N | ARG | A | 91 | 8354 | 12510 | 11923 | 195 | 2984 | −1204 | N |
| ATOM | 647 | CA | ARG | A | 91 | 112.159 | −72.037 | 69.194 | 1.00 | 85.57 | | C |
| ANISOU | 647 | CA | ARG | A | 91 | 8306 | 12132 | 12073 | −3 | 3154 | −1361 | C |
| ATOM | 648 | C | ARG | A | 91 | 112.732 | −72.690 | 70.457 | 1.00 | 86.31 | | C |
| ANISOU | 648 | C | ARG | A | 91 | 8615 | 11598 | 12582 | −146 | 3209 | −1290 | C |
| ATOM | 649 | O | ARG | A | 91 | 112.905 | −73.911 | 70.501 | 1.00 | 87.30 | | O |
| ANISOU | 649 | O | ARG | A | 91 | 8775 | 11509 | 12888 | −364 | 3426 | −1529 | O |
| ATOM | 650 | CB | ARG | A | 91 | 113.299 | −71.453 | 68.320 | 1.00 | 85.90 | | C |
| ANISOU | 650 | CB | ARG | A | 91 | 8384 | 12134 | 12121 | 200 | 3171 | −1106 | C |
| ATOM | 651 | CG | ARG | A | 91 | 114.379 | −72.462 | 67.879 | 1.00 | 96.92 | | C |
| ANISOU | 651 | CG | ARG | A | 91 | 9820 | 13247 | 13759 | 41 | 3349 | −1231 | C |
| ATOM | 652 | CD | ARG | A | 91 | 115.557 | −71.785 | 67.188 | 1.00 | 110.31 | | C |
| ANISOU | 652 | CD | ARG | A | 91 | 11565 | 14849 | 15500 | 238 | 3362 | −949 | C |
| ATOM | 653 | NE | ARG | A | 91 | 116.301 | −70.926 | 68.113 | 1.00 | 119.69 | | N |
| ANISOU | 653 | NE | ARG | A | 91 | 12914 | 15627 | 16934 | 357 | 3268 | −597 | N |
| ATOM | 654 | CZ | ARG | A | 91 | 117.584 | −71.071 | 68.415 | 1.00 | 134.64 | | C |
| ANISOU | 654 | CZ | ARG | A | 91 | 14892 | 17132 | 19133 | 338 | 3311 | −471 | C |
| ATOM | 655 | NH1 | ARG | A | 91 | 118.318 | −72.001 | 67.811 | 1.00 | 120.99 | | N |
| ANISOU | 655 | NH1 | ARG | A | 91 | 13140 | 15320 | 17513 | 251 | 3456 | −601 | N |
| ATOM | 656 | NH2 | ARG | A | 91 | 118.162 | −70.252 | 69.284 | 1.00 | 122.54 | | N |
| ANISOU | 656 | NH2 | ARG | A | 91 | 13441 | 15336 | 17783 | 408 | 3228 | −241 | N |
| ATOM | 657 | N | ARG | A | 92 | 113.027 | −71.876 | 71.466 | 1.00 | 78.57 | | N |
| ANISOU | 657 | N | ARG | A | 92 | 7773 | 10347 | 11733 | 0 | 3053 | −964 | N |
| ATOM | 658 | CA | ARG | A | 92 | 113.696 | −72.340 | 72.662 | 1.00 | 77.24 | | C |
| ANISOU | 658 | CA | ARG | A | 92 | 7772 | 9693 | 11884 | −39 | 3074 | −839 | C |
| ATOM | 659 | C | ARG | A | 92 | 112.784 | −72.712 | 73.806 | 1.00 | 82.50 | | C |
| ANISOU | 659 | C | ARG | A | 92 | 8501 | 10259 | 12586 | −134 | 3070 | −916 | C |
| ATOM | 660 | O | ARG | A | 92 | 113.038 | −73.717 | 74.474 | 1.00 | 83.68 | | O |
| ANISOU | 660 | O | ARG | A | 92 | 8766 | 10085 | 12944 | −213 | 3236 | −954 | O |
| ATOM | 661 | CB | ARG | A | 92 | 114.722 | −71.304 | 73.133 | 1.00 | 75.33 | | C |
| ANISOU | 661 | CB | ARG | A | 92 | 7599 | 9252 | 11770 | 142 | 2931 | −505 | C |
| ATOM | 662 | CG | ARG | A | 92 | 115.807 | −70.936 | 72.134 | 1.00 | 83.34 | | C |
| ANISOU | 662 | CG | ARG | A | 92 | 8577 | 10274 | 12815 | 226 | 2982 | −407 | C |
| ATOM | 663 | CD | ARG | A | 92 | 116.727 | −69.898 | 72.738 | 1.00 | 85.54 | | C |
| ANISOU | 663 | CD | ARG | A | 92 | 8899 | 10356 | 13245 | 331 | 2892 | −165 | C |
| ATOM | 664 | NE | ARG | A | 92 | 117.723 | −70.527 | 73.600 | 1.00 | 88.18 | | N |
| ANISOU | 664 | NE | ARG | A | 92 | 9253 | 10435 | 13816 | 315 | 2890 | −128 | N |
| ATOM | 665 | CZ | ARG | A | 92 | 119.029 | −70.478 | 73.378 | 1.00 | 101.70 | | C |
| ANISOU | 665 | CZ | ARG | A | 92 | 10917 | 12046 | 15678 | 354 | 2934 | −61 | C |
| ATOM | 666 | NH1 | ARG | A | 92 | 119.509 | −69.792 | 72.348 | 1.00 | 85.47 | | N |
| ANISOU | 666 | NH1 | ARG | A | 92 | 8825 | 10052 | 13600 | 378 | 3004 | −21 | N |
| ATOM | 667 | NH2 | ARG | A | 92 | 119.868 | −71.098 | 74.197 | 1.00 | 92.43 | | N |
| ANISOU | 667 | NH2 | ARG | A | 92 | 9722 | 10743 | 14657 | 403 | 2927 | −20 | N |
| ATOM | 668 | N | PHE | A | 93 | 111.752 | −71.909 | 74.068 | 1.00 | 77.76 | | N |
| ANISOU | 668 | N | PHE | A | 93 | 7845 | 9915 | 11785 | −88 | 2917 | −905 | N |
| ATOM | 669 | CA | PHE | A | 93 | 110.891 | −72.149 | 75.213 | 1.00 | 77.47 | | C |
| ANISOU | 669 | CA | PHE | A | 93 | 7872 | 9782 | 11783 | −168 | 2902 | −957 | C |
| ATOM | 670 | C | PHE | A | 93 | 109.425 | −72.501 | 74.872 | 1.00 | 88.62 | | C |
| ANISOU | 670 | C | PHE | A | 93 | 9126 | 11580 | 12966 | −337 | 2969 | −1307 | C |
| ATOM | 671 | O | PHE | A | 93 | 108.587 | −72.690 | 75.778 | 1.00 | 88.59 | | O |
| ANISOU | 671 | O | PHE | A | 93 | 9160 | 11523 | 12979 | −425 | 2980 | −1382 | O |
| ATOM | 672 | CB | PHE | A | 93 | 110.969 | −70.935 | 76.130 | 1.00 | 77.62 | | C |
| ANISOU | 672 | CB | PHE | A | 93 | 7965 | 9721 | 11807 | 12 | 2680 | −658 | C |
| ATOM | 673 | CG | PHE | A | 93 | 112.367 | −70.507 | 76.488 | 1.00 | 76.24 | | C |
| ANISOU | 673 | CG | PHE | A | 93 | 7871 | 9267 | 11829 | 131 | 2621 | −407 | C |
| ATOM | 674 | CD2 | PHE | A | 93 | 112.916 | −69.357 | 75.945 | 1.00 | 75.95 | | C |
| ANISOU | 674 | CD2 | PHE | A | 93 | 7806 | 9297 | 11754 | 259 | 2553 | −247 | C |
| ATOM | 675 | CD1 | PHE | A | 93 | 113.114 | −71.226 | 77.408 | 1.00 | 76.76 | | C |
| ANISOU | 675 | CD1 | PHE | A | 93 | 8029 | 9036 | 12103 | 137 | 2667 | −336 | C |
| ATOM | 676 | CE2 | PHE | A | 93 | 114.182 | −68.929 | 76.322 | 1.00 | 77.85 | | C |
| ANISOU | 676 | CE2 | PHE | A | 93 | 8077 | 9319 | 12183 | 312 | 2526 | −96 | C |
| ATOM | 677 | CE1 | PHE | A | 93 | 114.384 | −70.805 | 77.773 | 1.00 | 76.50 | | C |
| ANISOU | 677 | CE1 | PHE | A | 93 | 7993 | 8871 | 12200 | 250 | 2593 | −158 | C |
| ATOM | 678 | CZ | PHE | A | 93 | 114.909 | −69.659 | 77.229 | 1.00 | 75.62 | | C |
| ANISOU | 678 | CZ | PHE | A | 93 | 7821 | 8837 | 12072 | 298 | 2519 | −76 | C |
| ATOM | 679 | N | GLY | A | 94 | 109.131 | −72.594 | 73.577 | 1.00 | 89.70 | | N |
| ANISOU | 679 | N | GLY | A | 94 | 9058 | 12153 | 12871 | −382 | 3021 | −1544 | N |
| ATOM | 680 | CA | GLY | A | 94 | 107.801 | −72.951 | 73.096 | 1.00 | 91.49 | | C |
| ANISOU | 680 | CA | GLY | A | 94 | 9039 | 12901 | 12823 | −556 | 3087 | −1965 | C |
| ATOM | 681 | C | GLY | A | 94 | 106.902 | −71.788 | 72.724 | 1.00 | 98.33 | | C |
| ANISOU | 681 | C | GLY | A | 94 | 9710 | 14364 | 13286 | −313 | 2865 | −1883 | C |
| ATOM | 682 | O | GLY | A | 94 | 107.272 | −70.625 | 72.913 | 1.00 | 98.10 | | O |
| ANISOU | 682 | O | GLY | A | 94 | 9793 | 14253 | 13229 | −6 | 2697 | −1465 | O |
| ATOM | 683 | N | PRO | A | 95 | 105.690 | −72.078 | 72.197 | 1.00 | 96.58 | | N |
| ANISOU | 683 | N | PRO | A | 95 | 9180 | 14777 | 12738 | −436 | 2902 | −2300 | N |
| ATOM | 684 | CA | PRO | A | 95 | 104.787 | −70.982 | 71.821 | 1.00 | 96.31 | | C |
| ANISOU | 684 | CA | PRO | A | 95 | 8931 | 15403 | 12257 | −116 | 2707 | −2190 | C |
| ATOM | 685 | C | PRO | A | 95 | 104.109 | −70.329 | 73.022 | 1.00 | 97.14 | | C |
| ANISOU | 685 | C | PRO | A | 95 | 9152 | 15352 | 12405 | −16 | 2578 | −1978 | C |
| ATOM | 686 | O | PRO | A | 95 | 103.864 | −69.131 | 72.968 | 1.00 | 97.17 | | O |
| ANISOU | 686 | O | PRO | A | 95 | 9157 | 15575 | 12188 | 361 | 2434 | −1637 | O |
| ATOM | 687 | CB | PRO | A | 95 | 103.776 | −71.658 | 70.896 | 1.00 | 98.67 | | C |
| ANISOU | 687 | CB | PRO | A | 95 | 8800 | 16512 | 12178 | −311 | 2797 | −2783 | C |
| ATOM | 688 | CG | PRO | A | 95 | 103.713 | −73.072 | 71.382 | 1.00 | 103.59 | | C |
| ANISOU | 688 | CG | PRO | A | 95 | 9472 | 16740 | 13147 | −839 | 3071 | −3247 | C |
| ATOM | 689 | CD | PRO | A | 95 | 105.086 | −73.402 | 71.919 | 1.00 | 98.89 | | C |
| ANISOU | 689 | CD | PRO | A | 95 | 9286 | 15245 | 13044 | −867 | 3166 | −2915 | C |
| ATOM | 690 | N | THR | A | 96 | 103.817 | −71.106 | 74.096 | 1.00 | 90.11 | | N |
| ANISOU | 690 | N | THR | A | 96 | 8379 | 14059 | 11798 | −331 | 2675 | −2161 | N |
| ATOM | 691 | CA | THR | A | 96 | 103.169 | −70.612 | 75.313 | 1.00 | 88.07 | | C |
| ANISOU | 691 | CA | THR | A | 96 | 8235 | 13630 | 11598 | −276 | 2571 | −1998 | C |
| ATOM | 692 | C | THR | A | 96 | 104.005 | −69.536 | 75.998 | 1.00 | 88.72 | | C |
| ANISOU | 692 | C | THR | A | 96 | 8610 | 13242 | 11857 | 13 | 2420 | −1450 | C |
| ATOM | 693 | O | THR | A | 96 | 103.449 | −68.523 | 76.399 | 1.00 | 89.36 | | O |
| ANISOU | 693 | O | THR | A | 96 | 8702 | 13456 | 11793 | 246 | 2291 | −1232 | O |
| ATOM | 694 | CB | THR | A | 96 | 102.761 | −71.754 | 76.266 | 1.00 | 90.26 | | C |
| ANISOU | 694 | CB | THR | A | 96 | 8599 | 13551 | 12147 | −660 | 2771 | −2299 | C |
| ATOM | 695 | OG1 | THR | A | 96 | 103.790 | −72.744 | 76.331 | 1.00 | 88.53 | | O |
| ANISOU | 695 | OG1 | THR | A | 96 | 8580 | 12781 | 12276 | −848 | 2990 | −2328 | O |
| ATOM | 696 | CG2 | THR | A | 96 | 101.443 | −72.391 | 75.882 | 1.00 | 87.08 | | C |
| ANISOU | 696 | CG2 | THR | A | 96 | 7853 | 13728 | 11506 | −939 | 2908 | −2874 | C |
| ATOM | 697 | N | VAL | A | 97 | 105.328 | −69.730 | 76.099 | 1.00 | 81.50 | | N |
| ANISOU | 697 | N | VAL | A | 97 | 7906 | 11819 | 11241 | −3 | 2462 | −1260 | N |
| ATOM | 698 | CA | VAL | A | 97 | 106.219 | −68.757 | 76.723 | 1.00 | 79.69 | | C |
| ANISOU | 698 | CA | VAL | A | 97 | 7901 | 11187 | 11191 | 202 | 2354 | −841 | C |
| ATOM | 699 | C | VAL | A | 97 | 106.253 | −67.522 | 75.833 | 1.00 | 81.58 | | C |
| ANISOU | 699 | C | VAL | A | 97 | 8083 | 11726 | 11189 | 527 | 2307 | −604 | C |
| ATOM | 700 | O | VAL | A | 97 | 106.073 | −66.412 | 76.333 | 1.00 | 79.78 | | O |
| ANISOU | 700 | O | VAL | A | 97 | 7955 | 11420 | 10938 | 731 | 2253 | −344 | O |
| ATOM | 701 | CB | VAL | A | 97 | 107.620 | −69.364 | 77.006 | 1.00 | 83.40 | | C |
| ANISOU | 701 | CB | VAL | A | 97 | 8539 | 11151 | 11999 | 104 | 2423 | −755 | C |
| ATOM | 702 | CG1 | VAL | A | 97 | 108.587 | −68.319 | 77.542 | 1.00 | 83.14 | | C |
| ANISOU | 702 | CG1 | VAL | A | 97 | 8655 | 10814 | 12120 | 274 | 2326 | −418 | C |
| ATOM | 703 | CG2 | VAL | A | 97 | 107.522 | −70.527 | 77.982 | 1.00 | 83.30 | | C |
| ANISOU | 703 | CG2 | VAL | A | 97 | 8626 | 10827 | 12198 | −112 | 2536 | −900 | C |
| ATOM | 704 | N | ARG | A | 98 | 106.402 | −67.733 | 74.504 | 1.00 | 79.09 | | N |
| ANISOU | 704 | N | ARG | A | 98 | 7607 | 11772 | 10673 | 589 | 2374 | −704 | N |
| ATOM | 705 | CA | ARG | A | 98 | 106.430 | −66.680 | 73.482 | 1.00 | 79.33 | | C |
| ANISOU | 705 | CA | ARG | A | 98 | 7579 | 12142 | 10420 | 959 | 2397 | −458 | C |
| ATOM | 706 | C | ARG | A | 98 | 105.150 | −65.848 | 73.536 | 1.00 | 82.98 | | C |
| ANISOU | 706 | C | ARG | A | 98 | 7927 | 13073 | 10529 | 1242 | 2346 | −368 | C |
| ATOM | 707 | O | ARG | A | 98 | 105.237 | −64.623 | 73.503 | 1.00 | 83.45 | | O |
| ANISOU | 707 | O | ARG | A | 98 | 8119 | 13061 | 10527 | 1587 | 2404 | 9 | O |
| ATOM | 708 | CB | ARG | A | 98 | 106.642 | −67.278 | 72.076 | 1.00 | 81.50 | | C |
| ANISOU | 708 | CB | ARG | A | 98 | 7649 | 12840 | 10477 | 959 | 2473 | −657 | C |
| ATOM | 709 | CG | ARG | A | 98 | 106.737 | −66.245 | 70.948 | 1.00 | 97.46 | | C |
| ANISOU | 709 | CG | ARG | A | 98 | 9621 | 15238 | 12170 | 1406 | 2539 | −359 | C |
| ATOM | 710 | CD | ARG | A | 98 | 105.813 | −66.595 | 69.794 | 1.00 | 114.80 | | C |
| ANISOU | 710 | CD | ARG | A | 98 | 11444 | 18337 | 13837 | 1535 | 2530 | −632 | C |
| ATOM | 711 | NE | ARG | A | 98 | 105.694 | −65.503 | 68.822 | 1.00 | 129.28 | | N |
| ANISOU | 711 | NE | ARG | A | 98 | 13230 | 20626 | 15266 | 2090 | 2610 | −269 | N |
| ATOM | 712 | CZ | ARG | A | 98 | 106.069 | −65.580 | 67.547 | 1.00 | 148.30 | | C |
| ANISOU | 712 | CZ | ARG | A | 98 | 15501 | 23442 | 17405 | 2278 | 2694 | −266 | C |
| ATOM | 713 | NH1 | ARG | A | 98 | 106.589 | −66.702 | 67.066 | 1.00 | 140.81 | | N |
| ANISOU | 713 | NH1 | ARG | A | 98 | 14438 | 22501 | 16563 | 1915 | 2700 | −646 | N |
| ATOM | 714 | NH2 | ARG | A | 98 | 105.923 | −64.535 | 66.742 | 1.00 | 134.17 | | N |
| ANISOU | 714 | NH2 | ARG | A | 98 | 13701 | 22048 | 15228 | 2858 | 2814 | 135 | N |
| ATOM | 715 | N | ARG | A | 99 | 103.980 | −66.515 | 73.651 | 1.00 | 78.16 | | N |
| ANISOU | 715 | N | ARG | A | 99 | 7076 | 12915 | 9707 | 1091 | 2285 | −722 | N |
| ATOM | 716 | CA | ARG | A | 99 | 102.652 | −65.904 | 73.712 | 1.00 | 77.68 | | C |
| ANISOU | 716 | CA | ARG | A | 99 | 6833 | 13413 | 9269 | 1342 | 2222 | −709 | C |
| ATOM | 717 | C | ARG | A | 99 | 102.555 | −64.906 | 74.874 | 1.00 | 76.46 | | C |
| ANISOU | 717 | C | ARG | A | 99 | 6945 | 12803 | 9301 | 1489 | 2196 | −361 | C |
| ATOM | 718 | O | ARG | A | 99 | 102.288 | −63.728 | 74.633 | 1.00 | 76.93 | | O |
| ANISOU | 718 | O | ARG | A | 99 | 7054 | 13021 | 9154 | 1921 | 2254 | −12 | O |
| ATOM | 719 | CB | ARG | A | 99 | 101.553 | −66.983 | 73.873 | 1.00 | 83.17 | | C |
| ANISOU | 719 | CB | ARG | A | 99 | 7228 | 14548 | 9824 | 1006 | 2189 | −1251 | C |
| ATOM | 720 | CG | ARG | A | 99 | 101.083 | −67.696 | 72.606 | 1.00 | 102.65 | | C |
| ANISOU | 720 | CG | ARG | A | 99 | 9281 | 17822 | 11901 | 938 | 2229 | −1692 | C |
| ATOM | 721 | CD | ARG | A | 99 | 100.208 | −68.878 | 73.013 | 1.00 | 118.59 | | C |
| ANISOU | 721 | CD | ARG | A | 99 | 11073 | 20031 | 13956 | 448 | 2290 | −2302 | C |
| ATOM | 722 | NE | ARG | A | 99 | 99.348 | −69.359 | 71.932 | 1.00 | 132.45 | | N |
| ANISOU | 722 | NE | ARG | A | 99 | 12314 | 22783 | 15227 | 388 | 2318 | −2824 | N |
| ATOM | 723 | CZ | ARG | A | 99 | 99.398 | −70.583 | 71.415 | 1.00 | 149.94 | | C |
| ANISOU | 723 | CZ | ARG | A | 99 | 14330 | 25143 | 17498 | −70 | 2490 | −3408 | C |
| ATOM | 724 | NH1 | ARG | A | 99 | 100.280 | −71.468 | 71.865 | 1.00 | 139.05 | | N |
| ANISOU | 724 | NH1 | ARG | A | 99 | 13260 | 22926 | 16646 | −456 | 2671 | −3478 | N |
| ATOM | 725 | NH2 | ARG | A | 99 | 98.573 | −70.930 | 70.437 | 1.00 | 137.43 | | N |
| ANISOU | 725 | NH2 | ARG | A | 99 | 12219 | 24575 | 15424 | −129 | 2512 | −3940 | N |
| ATOM | 726 | N | ILE | A | 100 | 102.766 | −65.393 | 76.128 | 1.00 | 67.14 | | N |
| ANISOU | 726 | N | ILE | A | 100 | 5941 | 11070 | 8499 | 1151 | 2149 | −456 | N |
| ATOM | 727 | CA | ILE | A | 100 | 102.734 | −64.615 | 77.374 | 1.00 | 62.92 | | C |
| ANISOU | 727 | CA | ILE | A | 100 | 5643 | 10092 | 8172 | 1199 | 2117 | −216 | C |
| ATOM | 728 | C | ILE | A | 100 | 103.700 | −63.419 | 77.262 | 1.00 | 72.87 | | C |
| ANISOU | 728 | C | ILE | A | 100 | 7140 | 10972 | 9575 | 1450 | 2224 | 184 | C |
| ATOM | 729 | O | ILE | A | 100 | 103.281 | −62.303 | 77.562 | 1.00 | 74.83 | | O |
| ANISOU | 729 | O | ILE | A | 100 | 7490 | 11192 | 9751 | 1719 | 2294 | 436 | O |
| ATOM | 730 | CB | ILE | A | 100 | 103.124 | −65.476 | 78.617 | 1.00 | 61.28 | | C |
| ANISOU | 730 | CB | ILE | A | 100 | 5582 | 9364 | 8337 | 818 | 2070 | −370 | C |
| ATOM | 731 | CG1 | ILE | A | 100 | 102.293 | −66.727 | 78.783 | 1.00 | 57.82 | | C |
| ANISOU | 731 | CG1 | ILE | A | 100 | 4974 | 9147 | 7848 | 521 | 2080 | −773 | C |
| ATOM | 732 | CG2 | ILE | A | 100 | 103.115 | −64.662 | 79.890 | 1.00 | 59.83 | | C |
| ANISOU | 732 | CG2 | ILE | A | 100 | 5605 | 8804 | 8324 | 868 | 2026 | −161 | C |
| ATOM | 733 | CD1 | ILE | A | 100 | 102.984 | −67.761 | 79.721 | 1.00 | 38.95 | | C |
| ANISOU | 733 | CD1 | ILE | A | 100 | 2767 | 6200 | 5831 | 214 | 2137 | −864 | C |
| ATOM | 734 | N | VAL | A | 101 | 104.984 | −63.664 | 76.870 | 1.00 | 70.78 | | N |
| ANISOU | 734 | N | VAL | A | 101 | 6967 | 10390 | 9536 | 1342 | 2281 | 219 | N |
| ATOM | 735 | CA | VAL | A | 101 | 106.040 | −62.661 | 76.818 | 1.00 | 72.37 | | C |
| ANISOU | 735 | CA | VAL | A | 101 | 7380 | 10174 | 9943 | 1473 | 2431 | 508 | C |
| ATOM | 736 | C | VAL | A | 101 | 105.682 | −61.546 | 75.825 | 1.00 | 85.67 | | C |
| ANISOU | 736 | C | VAL | A | 101 | 9077 | 12135 | 11340 | 1930 | 2632 | 810 | C |
| ATOM | 737 | O | VAL | A | 101 | 105.808 | −60.376 | 76.197 | 1.00 | 84.92 | | O |
| ANISOU | 737 | O | VAL | A | 101 | 9185 | 11726 | 11354 | 2109 | 2821 | 1071 | O |
| ATOM | 738 | CB | VAL | A | 101 | 107.451 | −63.284 | 76.605 | 1.00 | 74.68 | | C |
| ANISOU | 738 | CB | VAL | A | 101 | 7716 | 10142 | 10517 | 1245 | 2444 | 433 | C |
| ATOM | 739 | CG1 | VAL | A | 101 | 108.491 | −62.233 | 76.246 | 1.00 | 74.30 | | C |
| ANISOU | 739 | CG1 | VAL | A | 101 | 7827 | 9773 | 10632 | 1380 | 2656 | 681 | C |
| ATOM | 740 | CG2 | VAL | A | 101 | 107.896 | −64.034 | 77.849 | 1.00 | 74.00 | | C |
| ANISOU | 740 | CG2 | VAL | A | 101 | 7683 | 9712 | 10722 | 925 | 2312 | 269 | C |
| ATOM | 741 | N | GLU | A | 102 | 105.177 | −61.889 | 74.611 | 1.00 | 89.87 | | N |
| ANISOU | 741 | N | GLU | A | 102 | 9387 | 13276 | 11483 | 2141 | 2629 | 768 | N |
| ATOM | 742 | CA | GLU | A | 102 | 104.764 | −60.897 | 73.603 | 1.00 | 93.05 | | C |
| ANISOU | 742 | CA | GLU | A | 102 | 9773 | 14065 | 11515 | 2685 | 2835 | 1100 | C |
| ATOM | 743 | C | GLU | A | 102 | 103.720 | −59.998 | 74.225 | 1.00 | 106.70 | | C |
| ANISOU | 743 | C | GLU | A | 102 | 11559 | 15885 | 13095 | 2971 | 2894 | 1297 | C |
| ATOM | 744 | O | GLU | A | 102 | 103.905 | −58.785 | 74.268 | 1.00 | 105.93 | | O |
| ANISOU | 744 | O | GLU | A | 102 | 11710 | 15475 | 13065 | 3287 | 3184 | 1673 | O |
| ATOM | 745 | CB | GLU | A | 102 | 104.193 | −61.576 | 72.345 | 1.00 | 94.34 | | C |
| ANISOU | 745 | CB | GLU | A | 102 | 9592 | 15058 | 11196 | 2850 | 2754 | 927 | C |
| ATOM | 746 | CG | GLU | A | 102 | 105.224 | −61.853 | 71.258 | 1.00 | 105.50 | | C |
| ANISOU | 746 | CG | GLU | A | 102 | 11002 | 16460 | 12622 | 2860 | 2855 | 953 | C |
| ATOM | 747 | CD | GLU | A | 102 | 104.699 | −62.535 | 70.006 | 1.00 | 122.52 | | C |
| ANISOU | 747 | CD | GLU | A | 102 | 12787 | 19488 | 14278 | 2997 | 2783 | 726 | C |
| ATOM | 748 | OE1 | GLU | A | 102 | 105.114 | −62.128 | 68.897 | 1.00 | 97.66 | | O |
| ANISOU | 748 | OE1 | GLU | A | 102 | 9632 | 16573 | 10900 | 3351 | 2951 | 960 | O |
| ATOM | 749 | OE2 | GLU | A | 102 | 103.884 | −63.480 | 70.129 | 1.00 | 123.28 | | O |
| ANISOU | 749 | OE2 | GLU | A | 102 | 12587 | 20045 | 14208 | 2738 | 2588 | 285 | O |
| ATOM | 750 | N | GLY | A | 103 | 102.692 | −60.625 | 74.793 | 1.00 | 112.19 | | N |
| ANISOU | 750 | N | GLY | A | 103 | 12052 | 16925 | 13651 | 2812 | 2661 | 1019 | N |
| ATOM | 751 | CA | GLY | A | 103 | 101.581 | −59.976 | 75.480 | 1.00 | 115.94 | | C |
| ANISOU | 751 | CA | GLY | A | 103 | 12530 | 17550 | 13974 | 3027 | 2664 | 1131 | C |
| ATOM | 752 | C | GLY | A | 103 | 101.964 | −59.011 | 76.587 | 1.00 | 128.81 | | C |
| ANISOU | 752 | C | GLY | A | 103 | 14513 | 18443 | 15987 | 2992 | 2822 | 1358 | C |
| ATOM | 753 | O | GLY | A | 103 | 101.440 | −57.892 | 76.607 | 1.00 | 128.88 | | O |
| ANISOU | 753 | O | GLY | A | 103 | 14645 | 18467 | 15858 | 3415 | 3049 | 1688 | O |
| ATOM | 754 | N | GLU | A | 104 | 102.878 | −59.432 | 77.517 | 1.00 | 131.27 | | N |
| ANISOU | 754 | N | GLU | A | 104 | 14975 | 18142 | 16761 | 2510 | 2731 | 1175 | N |
| ATOM | 755 | CA | GLU | A | 104 | 103.398 | −58.603 | 78.623 | 1.00 | 133.52 | | C |
| ANISOU | 755 | CA | GLU | A | 104 | 15542 | 17769 | 17423 | 2384 | 2868 | 1279 | C |
| ATOM | 756 | CB | GLU | A | 104 | 104.152 | −59.442 | 79.667 | 1.00 | 134.77 | | C |
| ANISOU | 756 | CB | GLU | A | 104 | 15718 | 17552 | 17935 | 1871 | 2653 | 985 | C |
| ATOM | 757 | C | GLU | A | 104 | 104.268 | −57.430 | 78.103 | 1.00 | 143.64 | | C |
| ANISOU | 757 | C | GLU | A | 104 | 17069 | 18648 | 18860 | 2610 | 3268 | 1590 | C |
| ATOM | 758 | O | GLU | A | 104 | 104.256 | −56.355 | 78.712 | 1.00 | 144.33 | | O |
| ANISOU | 758 | O | GLU | A | 104 | 17380 | 18342 | 19118 | 2695 | 3533 | 1749 | O |
| ATOM | 759 | N | THR | A | 105 | 104.985 | −57.624 | 76.964 | 1.00 | 142.97 | | N |
| ANISOU | 759 | N | THR | A | 105 | 16950 | 18651 | 18721 | 2702 | 3362 | 1664 | N |
| ATOM | 760 | CA | THR | A | 105 | 105.799 | −56.580 | 76.325 | 1.00 | 144.08 | | C |
| ANISOU | 760 | CA | THR | A | 105 | 17322 | 18424 | 18996 | 2930 | 3803 | 1962 | C |
| ATOM | 761 | CB | THR | A | 105 | 106.895 | −57.208 | 75.431 | 1.00 | 149.80 | | C |
| ANISOU | 761 | CB | THR | A | 105 | 17978 | 19142 | 19796 | 2787 | 3780 | 1882 | C |
| ATOM | 762 | OG1 | THR | A | 105 | 108.017 | −57.557 | 76.244 | 1.00 | 148.45 | | O |
| ANISOU | 762 | OG1 | THR | A | 105 | 17849 | 18490 | 20064 | 2284 | 3684 | 1617 | O |
| ATOM | 763 | CG2 | THR | A | 105 | 107.375 | −56.279 | 74.327 | 1.00 | 147.48 | | C |
| ANISOU | 763 | CG2 | THR | A | 105 | 17860 | 18728 | 19448 | 3183 | 4245 | 2247 | C |
| ATOM | 764 | C | THR | A | 105 | 104.890 | −55.550 | 75.612 | 1.00 | 151.91 | | C |
| ANISOU | 764 | C | THR | A | 105 | 18403 | 19690 | 19626 | 3585 | 4144 | 2392 | C |
| ATOM | 765 | O | THR | A | 105 | 105.073 | −54.345 | 75.806 | 1.00 | 151.71 | | O |
| ANISOU | 765 | O | THR | A | 105 | 18671 | 19188 | 19783 | 3779 | 4600 | 2660 | O |
| ATOM | 766 | N | LYS | A | 106 | 103.983 | −56.062 | 74.779 | 1.00 | 151.23 | | N |
| ANISOU | 766 | N | LYS | A | 106 | 18046 | 20396 | 19018 | 3930 | 3957 | 2437 | N |
| ATOM | 767 | CA | LYS | A | 106 | 103.052 | −55.237 | 73.975 | 1.00 | 152.28 | | C |
| ANISOU | 767 | CA | LYS | A | 106 | 18163 | 21028 | 18668 | 4649 | 4213 | 2846 | C |
| ATOM | 768 | CB | LYS | A | 106 | 102.234 | −56.145 | 73.054 | 1.00 | 154.61 | | C |
| ANISOU | 768 | CB | LYS | A | 106 | 18014 | 22349 | 18383 | 4841 | 3867 | 2679 | C |
| ATOM | 769 | CG | LYS | A | 106 | 102.434 | −55.901 | 71.567 | 1.00 | 167.10 | | C |
| ANISOU | 769 | CG | LYS | A | 106 | 19483 | 24384 | 19623 | 5209 | 3996 | 2853 | C |
| ATOM | 770 | CD | LYS | A | 106 | 101.502 | −56.709 | 70.694 | 1.00 | 177.02 | | C |
| ANISOU | 770 | CD | LYS | A | 106 | 20227 | 26709 | 20324 | 5259 | 3623 | 2525 | C |
| ATOM | 771 | CE | LYS | A | 106 | 100.068 | −56.699 | 71.173 | 1.00 | 188.20 | | C |
| ANISOU | 771 | CE | LYS | A | 106 | 21352 | 28983 | 21173 | 5729 | 3541 | 2584 | C |
| ATOM | 772 | NZ | LYS | A | 106 | 99.109 | −56.533 | 70.055 | 1.00 | 197.46 | | N |
| ANISOU | 772 | NZ | LYS | A | 106 | 22624 | 30495 | 21905 | 6630 | 3950 | 3208 | N |
| ATOM | 773 | C | LYS | A | 106 | 102.130 | −54.422 | 74.887 | 1.00 | 159.22 | | C |
| ANISOU | 773 | C | LYS | A | 106 | 19201 | 21743 | 19552 | 4879 | 4390 | 3038 | C |
| ATOM | 774 | O | LYS | A | 106 | 101.764 | −53.298 | 74.488 | 1.00 | 159.09 | | O |
| ANISOU | 774 | O | LYS | A | 106 | 19395 | 21670 | 19382 | 5471 | 4860 | 3507 | O |
| ATOM | 775 | N | VAL | A | 107 | 101.780 | −54.952 | 76.065 | 1.00 | 157.41 | | N |
| ANISOU | 775 | N | VAL | A | 107 | 18889 | 21421 | 19496 | 4441 | 4058 | 2698 | N |
| ATOM | 776 | CA | VAL | A | 107 | 100.832 | −54.243 | 76.975 | 1.00 | 157.85 | | C |
| ANISOU | 776 | CA | VAL | A | 107 | 19056 | 21354 | 19564 | 4576 | 4153 | 2798 | C |
| ATOM | 777 | CB | VAL | A | 107 | 99.958 | −55.242 | 77.750 | 1.00 | 160.88 | | C |
| ANISOU | 777 | CB | VAL | A | 107 | 19157 | 22102 | 19866 | 4210 | 3650 | 2387 | C |
| ATOM | 778 | CG1 | VAL | A | 107 | 100.718 | −55.946 | 78.856 | 1.00 | 160.33 | | C |
| ANISOU | 778 | CG1 | VAL | A | 107 | 19148 | 21492 | 20277 | 3501 | 3405 | 1998 | C |
| ATOM | 779 | CG2 | VAL | A | 107 | 98.695 | −54.592 | 78.277 | 1.00 | 160.65 | | C |
| ANISOU | 779 | CG2 | VAL | A | 107 | 19151 | 22226 | 19662 | 4520 | 3735 | 2542 | C |
| ATOM | 780 | C | VAL | A | 107 | 101.527 | −53.223 | 77.889 | 1.00 | 165.42 | | C |
| ANISOU | 780 | C | VAL | A | 107 | 20433 | 21363 | 21054 | 4395 | 4570 | 2901 | C |
| ATOM | 781 | O | VAL | A | 107 | 101.463 | −53.385 | 79.098 | 1.00 | 164.86 | | O |
| ANISOU | 781 | O | VAL | A | 107 | 20418 | 20941 | 21282 | 3973 | 4429 | 2642 | O |
| ATOM | 782 | N | SER | A | 108 | 102.242 | −52.257 | 77.307 | 1.00 | 164.96 | | N |
| ANISOU | 782 | N | SER | A | 108 | 20661 | 20921 | 21096 | 4728 | 5133 | 3267 | N |
| ATOM | 783 | CA | SER | A | 108 | 102.840 | −51.074 | 77.995 | 1.00 | 166.00 | | C |
| ANISOU | 783 | CA | SER | A | 108 | 21190 | 20153 | 21729 | 4579 | 5689 | 3348 | C |
| ATOM | 784 | C | SER | A | 108 | 104.044 | −51.362 | 78.908 | 1.00 | 172.81 | | C |
| ANISOU | 784 | C | SER | A | 108 | 22059 | 20494 | 23108 | 3815 | 5529 | 2887 | C |
| ATOM | 785 | O | SER | A | 108 | 104.525 | −50.405 | 79.546 | 1.00 | 172.52 | | O |
| ANISOU | 785 | O | SER | A | 108 | 22264 | 19851 | 23434 | 3684 | 6006 | 2913 | O |
| ATOM | 786 | CB | SER | A | 108 | 101.801 | −50.216 | 78.666 | 1.00 | 169.23 | | C |
| ANISOU | 786 | CB | SER | A | 108 | 21794 | 20309 | 22196 | 4755 | 5964 | 3479 | C |
| ATOM | 787 | O | LYS | A | 109 | 104.757 | −52.353 | 81.872 | 1.00 | 175.22 | | O |
| ANISOU | 787 | O | LYS | A | 109 | 22295 | 20101 | 24179 | 2473 | 5012 | 1904 | O |
| ATOM | 788 | N | LYS | A | 109 | 104.514 | −52.609 | 78.978 | 1.00 | 171.09 | | N |
| ANISOU | 788 | N | LYS | A | 109 | 21567 | 20535 | 22905 | 3344 | 4907 | 2473 | N |
| ATOM | 789 | CA | LYS | A | 109 | 105.731 | −53.018 | 79.744 | 1.00 | 171.35 | | C |
| ANISOU | 789 | CA | LYS | A | 109 | 21515 | 20288 | 23303 | 2698 | 4634 | 2041 | C |
| ATOM | 790 | C | LYS | A | 109 | 105.792 | −52.591 | 81.227 | 1.00 | 175.79 | | C |
| ANISOU | 790 | C | LYS | A | 109 | 22235 | 20282 | 24274 | 2316 | 4829 | 1805 | C |
| ATOM | 791 | CB | LYS | A | 109 | 106.987 | −52.528 | 79.012 | 1.00 | 173.75 | | C |
| ANISOU | 791 | CB | LYS | A | 109 | 21800 | 20486 | 23731 | 2591 | 4722 | 2023 | C |
| ATOM | 792 | O | LEU | A | 110 | 109.378 | −51.080 | 82.392 | 1.00 | 176.17 | | O |
| ANISOU | 792 | O | LEU | A | 110 | 22578 | 18884 | 25473 | 1333 | 5883 | 1188 | O |
| ATOM | 793 | N | LEU | A | 110 | 107.029 | −52.588 | 81.732 | 1.00 | 172.44 | | N |
| ANISOU | 793 | N | LEU | A | 110 | 21753 | 19579 | 24187 | 1814 | 4763 | 1458 | N |
| ATOM | 794 | CA | LEU | A | 110 | 107.462 | −52.246 | 83.112 | 1.00 | 172.06 | | C |
| ANISOU | 794 | CA | LEU | A | 110 | 21758 | 19134 | 24484 | 1394 | 4900 | 1125 | C |
| ATOM | 795 | C | LEU | A | 110 | 108.329 | −50.979 | 83.053 | 1.00 | 176.22 | | C |
| ANISOU | 795 | C | LEU | A | 110 | 22541 | 19060 | 25355 | 1336 | 5618 | 1132 | C |
| ATOM | 796 | CB | LEU | A | 110 | 108.288 | −53.444 | 83.598 | 1.00 | 171.97 | | C |
| ANISOU | 796 | CB | LEU | A | 110 | 21487 | 19263 | 24593 | 922 | 4454 | 718 | C |
| ATOM | 797 | O | TYR | A | 111 | 109.044 | −46.770 | 82.278 | 1.00 | 174.67 | | O |
| ANISOU | 797 | O | TYR | A | 111 | 23329 | 16973 | 26063 | 1505 | 8129 | 1407 | O |
| ATOM | 798 | N | TYR | A | 111 | 107.902 | −49.892 | 83.736 | 1.00 | 172.26 | | N |
| ANISOU | 798 | N | TYR | A | 111 | 22207 | 18209 | 25034 | 1246 | 5967 | 1032 | N |
| ATOM | 799 | CA | TYR | A | 111 | 108.454 | −48.528 | 83.823 | 1.00 | 171.79 | | C |
| ANISOU | 799 | CA | TYR | A | 111 | 22418 | 17493 | 25363 | 1111 | 6756 | 947 | C |
| ATOM | 800 | C | TYR | A | 111 | 108.254 | −47.684 | 82.539 | 1.00 | 174.83 | | C |
| ANISOU | 800 | C | TYR | A | 111 | 23134 | 17566 | 25726 | 1645 | 7429 | 1471 | C |
| ATOM | 801 | CB | TYR | A | 111 | 109.943 | −48.554 | 84.252 | 1.00 | 173.00 | | C |
| ANISOU | 801 | CB | TYR | A | 111 | 22397 | 17459 | 25874 | 484 | 6813 | 401 | C |
| ATOM | 802 | CG | TYR | A | 111 | 110.205 | −48.437 | 85.741 | 1.00 | 174.45 | | C |
| ANISOU | 802 | CG | TYR | A | 111 | 22408 | 17647 | 26228 | −35 | 6672 | −157 | C |
| ATOM | 803 | CD2 | TYR | A | 111 | 111.117 | −47.508 | 86.235 | 1.00 | 175.00 | | C |
| ANISOU | 803 | CD2 | TYR | A | 111 | 22497 | 17290 | 26704 | −512 | 7224 | −628 | C |
| ATOM | 804 | CD1 | TYR | A | 111 | 109.627 | −49.325 | 86.645 | 1.00 | 176.32 | | C |
| ANISOU | 804 | CD1 | TYR | A | 111 | 22421 | 18365 | 26208 | −77 | 5990 | −263 | C |
| ATOM | 805 | CE2 | TYR | A | 111 | 111.406 | −47.430 | 87.596 | 1.00 | 175.64 | | C |
| ANISOU | 805 | CE2 | TYR | A | 111 | 22350 | 17499 | 26886 | −998 | 7069 | −1203 | C |
| ATOM | 806 | CE1 | TYR | A | 111 | 109.900 | −49.250 | 88.011 | 1.00 | 176.57 | | C |
| ANISOU | 806 | CE1 | TYR | A | 111 | 22267 | 18482 | 26338 | −509 | 5844 | −763 | C |
| ATOM | 807 | CZ | TYR | A | 111 | 110.796 | −48.303 | 88.481 | 1.00 | 181.64 | | C |
| ANISOU | 807 | CZ | TYR | A | 111 | 22896 | 18777 | 27341 | −965 | 6361 | −1244 | C |
| ATOM | 808 | OH | TYR | A | 111 | 111.088 | −48.228 | 89.820 | 1.00 | 180.65 | | O |
| ANISOU | 808 | OH | TYR | A | 111 | 22531 | 18845 | 27263 | −1392 | 6209 | −1791 | O |
| ATOM | 809 | O | LYS | A | 112 | 108.591 | −46.038 | 79.483 | 1.00 | 165.09 | | O |
| ANISOU | 809 | O | LYS | A | 112 | 22597 | 15656 | 24474 | 2793 | 9013 | 2609 | O |
| ATOM | 810 | N | LYS | A | 112 | 107.177 | −47.971 | 81.768 | 1.00 | 170.32 | | N |
| ANISOU | 810 | N | LYS | A | 112 | 22592 | 17403 | 24720 | 2269 | 7258 | 1975 | N |
| ATOM | 811 | CA | LYS | A | 112 | 106.777 | −47.286 | 80.526 | 1.00 | 190.23 | | C |
| ANISOU | 811 | CA | LYS | A | 112 | 25384 | 19833 | 27064 | 2945 | 7815 | 2565 | C |
| ATOM | 812 | C | LYS | A | 112 | 107.931 | −47.082 | 79.514 | 1.00 | 209.55 | | C |
| ANISOU | 812 | C | LYS | A | 112 | 27927 | 22003 | 29689 | 2902 | 8193 | 2622 | C |
| ATOM | 813 | CB | LYS | A | 112 | 106.058 | −45.956 | 80.828 | 1.00 | 192.72 | | C |
| ANISOU | 813 | CB | LYS | A | 112 | 26073 | 19670 | 27482 | 3289 | 8545 | 2844 | C |
| ATOM | 814 | O | ARG | A | 121 | 97.800 | −43.882 | 77.927 | 1.00 | 192.84 | | O |
| ANISOU | 814 | O | ARG | A | 121 | 25825 | 23087 | 24358 | 7216 | 7704 | 5083 | O |
| ATOM | 815 | N | ARG | A | 121 | 98.427 | −41.301 | 77.070 | 1.00 | 188.15 | | N |
| ANISOU | 815 | N | ARG | A | 121 | 25809 | 21564 | 24114 | 7137 | 8308 | 5169 | N |
| ATOM | 816 | CA | ARG | A | 121 | 97.524 | −41.493 | 78.202 | 1.00 | 187.79 | | C |
| ANISOU | 816 | CA | ARG | A | 121 | 25713 | 21586 | 24051 | 7238 | 8299 | 5172 | C |
| ATOM | 817 | C | ARG | A | 121 | 97.520 | −42.958 | 78.700 | 1.00 | 193.20 | | C |
| ANISOU | 817 | C | ARG | A | 121 | 26074 | 22683 | 24652 | 7016 | 7841 | 4954 | C |
| ATOM | 818 | CB | ARG | A | 121 | 96.105 | −40.990 | 77.843 | 1.00 | 185.42 | | C |
| ANISOU | 818 | CB | ARG | A | 121 | 25291 | 21845 | 23316 | 7771 | 8140 | 5406 | C |
| ATOM | 819 | CG | ARG | A | 121 | 95.122 | −40.857 | 79.015 | 1.00 | 189.89 | | C |
| ANISOU | 819 | CG | ARG | A | 121 | 25749 | 22480 | 23919 | 7540 | 7815 | 5129 | C |
| ATOM | 820 | CD | ARG | A | 121 | 95.391 | −39.667 | 79.922 | 1.00 | 192.39 | | C |
| ANISOU | 820 | CD | ARG | A | 121 | 26345 | 22053 | 24702 | 7016 | 7952 | 4812 | C |
| ATOM | 821 | NE | ARG | A | 121 | 94.484 | −39.648 | 81.066 | 1.00 | 195.02 | | N |
| ANISOU | 821 | NE | ARG | A | 121 | 26595 | 22453 | 25050 | 6906 | 7756 | 4636 | N |
| ATOM | 822 | O | ARG | A | 122 | 95.713 | −46.383 | 80.563 | 1.00 | 194.86 | | O |
| ANISOU | 822 | O | ARG | A | 122 | 25411 | 24423 | 24203 | 7398 | 7066 | 4981 | O |
| ATOM | 823 | N | ARG | A | 122 | 97.226 | −43.147 | 80.008 | 1.00 | 190.71 | | N |
| ANISOU | 823 | N | ARG | A | 122 | 25860 | 22053 | 24546 | 7033 | 8154 | 5000 | N |
| ATOM | 824 | CA | ARG | A | 122 | 97.130 | −44.443 | 80.695 | 1.00 | 190.94 | | C |
| ANISOU | 824 | CA | ARG | A | 122 | 25694 | 22318 | 24536 | 7089 | 8095 | 5045 | C |
| ATOM | 825 | C | ARG | A | 122 | 95.855 | −45.188 | 80.277 | 1.00 | 194.74 | | C |
| ANISOU | 825 | C | ARG | A | 122 | 25688 | 23942 | 24361 | 7387 | 7346 | 5023 | C |
| ATOM | 826 | CB | ARG | A | 122 | 97.164 | −44.246 | 82.218 | 1.00 | 192.09 | | C |
| ANISOU | 826 | CB | ARG | A | 122 | 26018 | 21824 | 25142 | 6652 | 8313 | 4785 | C |
| ATOM | 827 | N | ALA | A | 123 | 94.930 | −44.474 | 79.600 | 1.00 | 190.47 | | N |
| ANISOU | 827 | N | ALA | A | 123 | 25117 | 23813 | 23441 | 7915 | 7371 | 5283 | N |
| ATOM | 828 | CA | ALA | A | 123 | 93.693 | −45.029 | 79.063 | 1.00 | 189.83 | | C |
| ANISOU | 828 | CA | ALA | A | 123 | 24591 | 24817 | 22720 | 8359 | 6905 | 5355 | C |
| ATOM | 829 | C | ALA | A | 123 | 94.032 | −46.078 | 77.990 | 1.00 | 192.49 | | C |
| ANISOU | 829 | C | ALA | A | 123 | 24567 | 25832 | 22737 | 8314 | 6469 | 5224 | C |
| ATOM | 830 | O | ALA | A | 123 | 93.396 | −47.127 | 77.969 | 1.00 | 192.28 | | O |
| ANISOU | 830 | O | ALA | A | 123 | 24108 | 26675 | 22275 | 8512 | 6107 | 5175 | O |
| ATOM | 831 | CB | ALA | A | 123 | 92.836 | −43.923 | 78.471 | 1.00 | 190.59 | | C |
| ANISOU | 831 | CB | ALA | A | 123 | 24692 | 25042 | 22682 | 8412 | 6725 | 5285 | C |
| ATOM | 832 | N | GLU | A | 124 | 95.068 | −45.819 | 77.148 | 1.00 | 187.57 | | N |
| ANISOU | 832 | N | GLU | A | 124 | 24180 | 24886 | 22202 | 8544 | 6924 | 5538 | N |
| ATOM | 833 | CA | GLU | A | 124 | 95.536 | −46.728 | 76.092 | 1.00 | 186.42 | | C |
| ANISOU | 833 | CA | GLU | A | 124 | 23768 | 25338 | 21727 | 8734 | 6761 | 5610 | C |
| ATOM | 834 | C | GLU | A | 124 | 96.256 | −47.966 | 76.645 | 1.00 | 188.90 | | C |
| ANISOU | 834 | C | GLU | A | 124 | 23930 | 25633 | 22212 | 8378 | 6601 | 5418 | C |
| ATOM | 835 | O | GLU | A | 124 | 96.224 | −49.009 | 75.992 | 1.00 | 189.12 | | O |
| ANISOU | 835 | O | GLU | A | 124 | 23540 | 26410 | 21906 | 8264 | 6084 | 5169 | O |
| ATOM | 836 | CB | GLU | A | 124 | 96.430 | −45.995 | 75.079 | 1.00 | 187.60 | | C |
| ANISOU | 836 | CB | GLU | A | 124 | 24174 | 25002 | 22102 | 8512 | 6900 | 5574 | C |
| ATOM | 837 | N | ASP | A | 125 | 96.908 | −47.849 | 77.830 | 1.00 | 183.30 | | N |
| ANISOU | 837 | N | ASP | A | 125 | 23482 | 24001 | 22164 | 7638 | 6633 | 5073 | N |
| ATOM | 838 | CA | ASP | A | 125 | 97.641 | −48.936 | 78.508 | 1.00 | 181.87 | | C |
| ANISOU | 838 | CA | ASP | A | 125 | 23131 | 23619 | 22355 | 6691 | 6056 | 4431 | C |
| ATOM | 839 | C | ASP | A | 125 | 96.703 | −49.850 | 79.312 | 1.00 | 182.53 | | C |
| ANISOU | 839 | C | ASP | A | 125 | 22846 | 24245 | 22263 | 6373 | 5410 | 4028 | C |
| ATOM | 840 | O | ASP | A | 125 | 97.023 | −51.019 | 79.555 | 1.00 | 182.11 | | O |
| ANISOU | 840 | O | ASP | A | 125 | 22537 | 24359 | 22297 | 5778 | 4871 | 3559 | O |
| ATOM | 841 | CB | ASP | A | 125 | 98.752 | −48.368 | 79.406 | 1.00 | 183.68 | | C |
| ANISOU | 841 | CB | ASP | A | 125 | 23749 | 22746 | 23293 | 6106 | 6400 | 4236 | C |
| ATOM | 842 | N | LEU | A | 126 | 95.552 | −49.296 | 79.731 | 1.00 | 176.32 | | N |
| ANISOU | 842 | N | LEU | A | 126 | 22054 | 23695 | 21242 | 6788 | 5525 | 4228 | N |
| ATOM | 843 | CA | LEU | A | 126 | 94.496 | −50.004 | 80.444 | 1.00 | 174.56 | | C |
| ANISOU | 843 | CA | LEU | A | 126 | 21492 | 24023 | 20808 | 6595 | 5013 | 3907 | C |
| ATOM | 844 | C | LEU | A | 126 | 93.583 | −50.624 | 79.400 | 1.00 | 174.84 | | C |
| ANISOU | 844 | C | LEU | A | 126 | 21042 | 25239 | 20152 | 7064 | 4714 | 3948 | C |
| ATOM | 845 | O | LEU | A | 126 | 92.944 | −51.635 | 79.677 | 1.00 | 174.60 | | O |
| ANISOU | 845 | O | LEU | A | 126 | 20614 | 25793 | 19935 | 6739 | 4200 | 3524 | O |
| ATOM | 846 | CB | LEU | A | 126 | 93.703 | −49.035 | 81.323 | 1.00 | 174.58 | | C |
| ANISOU | 846 | CB | LEU | A | 126 | 21714 | 23747 | 20873 | 6863 | 5329 | 4116 | C |
| ATOM | 847 | N | ARG | A | 127 | 93.522 | −50.012 | 78.196 | 1.00 | 168.42 | | N |
| ANISOU | 847 | N | ARG | A | 127 | 20248 | 24803 | 18943 | 7833 | 5077 | 4443 | N |
| ATOM | 848 | CA | ARG | A | 127 | 92.740 | −50.511 | 77.065 | 1.00 | 166.72 | | C |
| ANISOU | 848 | CA | ARG | A | 127 | 19528 | 25827 | 17990 | 8357 | 4836 | 4490 | C |
| ATOM | 849 | C | ARG | A | 127 | 93.405 | −51.775 | 76.526 | 1.00 | 166.78 | | C |
| ANISOU | 849 | C | ARG | A | 127 | 19245 | 26109 | 18015 | 7790 | 4388 | 4015 | C |
| ATOM | 850 | O | ARG | A | 127 | 92.717 | −52.776 | 76.343 | 1.00 | 166.65 | | O |
| ANISOU | 850 | O | ARG | A | 127 | 18720 | 26956 | 17643 | 7607 | 3922 | 3591 | O |
| ATOM | 851 | CB | ARG | A | 127 | 92.609 | −49.455 | 75.954 | 1.00 | 166.85 | | C |
| ANISOU | 851 | CB | ARG | A | 127 | 19687 | 26133 | 17573 | 9394 | 5404 | 5207 | C |
| ATOM | 852 | N | GLN | A | 128 | 94.750 | −51.744 | 76.329 | 1.00 | 159.93 | | N |
| ANISOU | 852 | N | GLN | A | 128 | 18697 | 24473 | 17597 | 7464 | 4562 | 4040 | N |
| ATOM | 853 | CA | GLN | A | 128 | 95.569 | −52.869 | 75.840 | 1.00 | 157.81 | | C |
| ANISOU | 853 | CA | GLN | A | 128 | 18235 | 24297 | 17428 | 6926 | 4216 | 3635 | C |
| ATOM | 854 | C | GLN | A | 128 | 95.560 | −54.035 | 76.816 | 1.00 | 156.35 | | C |
| ANISOU | 854 | C | GLN | A | 128 | 17871 | 23988 | 17547 | 6076 | 3712 | 3001 | C |
| ATOM | 855 | O | GLN | A | 128 | 95.686 | −55.185 | 76.393 | 1.00 | 156.12 | | O |
| ANISOU | 855 | O | GLN | A | 128 | 17520 | 24387 | 17410 | 5717 | 3361 | 2594 | O |
| ATOM | 856 | CB | GLN | A | 128 | 97.016 | −52.427 | 75.557 | 1.00 | 159.11 | | C |
| ANISOU | 856 | CB | GLN | A | 128 | 18818 | 23585 | 18051 | 6787 | 4575 | 3833 | C |
| ATOM | 857 | N | MET | A | 129 | 95.408 | −53.728 | 78.119 | 1.00 | 148.49 | | N |
| ANISOU | 857 | N | MET | A | 129 | 17098 | 22395 | 16927 | 5778 | 3726 | 2928 | N |
| ATOM | 858 | CA | MET | A | 129 | 95.325 | −54.697 | 79.202 | 1.00 | 146.26 | | C |
| ANISOU | 858 | CA | MET | A | 129 | 16706 | 21938 | 16927 | 5063 | 3326 | 2414 | C |
| ATOM | 859 | C | MET | A | 129 | 94.132 | −55.617 | 78.951 | 1.00 | 146.56 | | C |
| ANISOU | 859 | C | MET | A | 129 | 16229 | 22964 | 16491 | 5065 | 2967 | 2076 | C |
| ATOM | 860 | O | MET | A | 129 | 94.264 | −56.833 | 79.089 | 1.00 | 146.25 | | O |
| ANISOU | 860 | O | MET | A | 129 | 15979 | 23040 | 16551 | 4510 | 2653 | 1600 | O |
| ATOM | 861 | CB | MET | A | 129 | 95.216 | −53.964 | 80.542 | 1.00 | 148.35 | | C |
| ANISOU | 861 | CB | MET | A | 129 | 17290 | 21517 | 17559 | 4924 | 3478 | 2485 | C |
| ATOM | 862 | N | PHE | A | 130 | 92.996 | −55.037 | 78.503 | 1.00 | 140.05 | | N |
| ANISOU | 862 | N | PHE | A | 130 | 15189 | 22888 | 15136 | 5716 | 3066 | 2318 | N |
| ATOM | 863 | CA | PHE | A | 130 | 91.769 | −55.756 | 78.165 | 1.00 | 138.17 | | C |
| ANISOU | 863 | CA | PHE | A | 130 | 14387 | 23748 | 14363 | 5795 | 2776 | 1981 | C |
| ATOM | 864 | C | PHE | A | 130 | 91.938 | −56.541 | 76.868 | 1.00 | 136.85 | | C |
| ANISOU | 864 | C | PHE | A | 130 | 13830 | 24346 | 13819 | 5823 | 2630 | 1752 | C |
| ATOM | 865 | O | PHE | A | 130 | 91.608 | −57.720 | 76.831 | 1.00 | 135.67 | | O |
| ANISOU | 865 | O | PHE | A | 130 | 13303 | 24655 | 13592 | 5335 | 2334 | 1173 | O |
| ATOM | 866 | CB | PHE | A | 130 | 90.571 | −54.795 | 78.079 | 1.00 | 140.23 | | C |
| ANISOU | 866 | CB | PHE | A | 130 | 14521 | 24622 | 14137 | 6561 | 2951 | 2348 | C |
| ATOM | 867 | CG | PHE | A | 130 | 89.205 | −55.418 | 78.246 | 1.00 | 142.28 | | C |
| ANISOU | 867 | CG | PHE | A | 130 | 14234 | 25857 | 13971 | 6519 | 2656 | 1934 | C |
| ATOM | 868 | CD1 | PHE | A | 130 | 89.061 | −56.718 | 78.720 | 1.00 | 145.74 | | C |
| ANISOU | 868 | CD1 | PHE | A | 130 | 14415 | 26370 | 14592 | 5732 | 2319 | 1259 | C |
| ATOM | 869 | CD2 | PHE | A | 130 | 88.056 | −54.690 | 77.961 | 1.00 | 145.38 | | C |
| ANISOU | 869 | CD2 | PHE | A | 130 | 14373 | 27079 | 13788 | 7278 | 2768 | 2221 | C |
| ATOM | 870 | CE1 | PHE | A | 130 | 87.800 | −57.290 | 78.872 | 1.00 | 147.27 | | C |
| ANISOU | 870 | CE1 | PHE | A | 130 | 14093 | 27442 | 14420 | 5641 | 2110 | 821 | C |
| ATOM | 871 | CE2 | PHE | A | 130 | 86.792 | −55.261 | 78.122 | 1.00 | 148.74 | | C |
| ANISOU | 871 | CE2 | PHE | A | 130 | 14242 | 28463 | 13810 | 7212 | 2501 | 1785 | C |
| ATOM | 872 | CZ | PHE | A | 130 | 86.671 | −56.558 | 78.570 | 1.00 | 146.96 | | C |
| ANISOU | 872 | CZ | PHE | A | 130 | 13755 | 28288 | 13797 | 6362 | 2180 | 1060 | C |
| ATOM | 873 | N | ILE | A | 131 | 92.474 | −55.901 | 75.818 | 1.00 | 130.78 | | N |
| ANISOU | 873 | N | ILE | A | 131 | 13170 | 23682 | 12839 | 6371 | 2883 | 2187 | N |
| ATOM | 874 | CA | ILE | A | 131 | 92.757 | −56.506 | 74.505 | 1.00 | 129.25 | | C |
| ANISOU | 874 | CA | ILE | A | 131 | 12647 | 24192 | 12270 | 6470 | 2792 | 2037 | C |
| ATOM | 875 | C | ILE | A | 131 | 93.645 | −57.780 | 74.634 | 1.00 | 130.91 | | C |
| ANISOU | 875 | C | ILE | A | 131 | 12848 | 23972 | 12920 | 5602 | 2550 | 1492 | C |
| ATOM | 876 | O | ILE | A | 131 | 93.407 | −58.775 | 73.937 | 1.00 | 129.59 | | O |
| ANISOU | 876 | O | ILE | A | 131 | 12235 | 24547 | 12456 | 5385 | 2339 | 1024 | O |
| ATOM | 877 | CB | ILE | A | 131 | 93.336 | −55.413 | 73.565 | 1.00 | 131.73 | | C |
| ANISOU | 877 | CB | ILE | A | 131 | 13237 | 24412 | 12402 | 7226 | 3204 | 2717 | C |
| ATOM | 878 | CG1 | ILE | A | 131 | 92.194 | −54.493 | 73.078 | 1.00 | 131.87 | | C |
| ANISOU | 878 | CG1 | ILE | A | 131 | 13051 | 25326 | 11729 | 8205 | 3405 | 3171 | C |
| ATOM | 879 | CG2 | ILE | A | 131 | 94.097 | −55.998 | 72.380 | 1.00 | 131.86 | | C |
| ANISOU | 879 | CG2 | ILE | A | 131 | 13098 | 24745 | 12259 | 7183 | 3156 | 2601 | C |
| ATOM | 880 | CD1 | ILE | A | 131 | 92.422 | −53.017 | 73.219 | 1.00 | 136.00 | | C |
| ANISOU | 880 | CD1 | ILE | A | 131 | 14116 | 25170 | 12385 | 8870 | 3965 | 3940 | C |
| ATOM | 881 | N | ALA | A | 132 | 94.523 | −57.812 | 75.636 | 1.00 | 126.60 | | N |
| ANISOU | 881 | N | ALA | A | 132 | 12770 | 22271 | 13059 | 5119 | 2605 | 1530 | N |
| ATOM | 882 | CA | ALA | A | 132 | 95.371 | −59.004 | 75.877 | 1.00 | 125.75 | | C |
| ANISOU | 882 | CA | ALA | A | 132 | 12725 | 21644 | 13410 | 4366 | 2425 | 1105 | C |
| ATOM | 883 | C | ALA | A | 132 | 94.557 | −60.046 | 76.647 | 1.00 | 128.36 | | C |
| ANISOU | 883 | C | ALA | A | 132 | 12796 | 22153 | 13821 | 3784 | 2155 | 521 | C |
| ATOM | 884 | O | ALA | A | 132 | 94.861 | −61.240 | 76.547 | 1.00 | 127.70 | | O |
| ANISOU | 884 | O | ALA | A | 132 | 12613 | 21985 | 13925 | 3258 | 2035 | 101 | O |
| ATOM | 885 | CB | ALA | A | 132 | 96.612 | −58.610 | 76.628 | 1.00 | 126.45 | | C |
| ANISOU | 885 | CB | ALA | A | 132 | 13343 | 20578 | 14123 | 4132 | 2586 | 1352 | C |
| ATOM | 886 | O | MET | A | 133 | 91.781 | −62.723 | 77.626 | 1.00 | 124.20 | | O |
| ANISOU | 886 | O | MET | A | 133 | 11076 | 23359 | 12754 | 2844 | 1715 | −1082 | O |
| ATOM | 887 | N | MET | A | 133 | 93.419 | −59.629 | 77.219 | 1.00 | 123.98 | | N |
| ANISOU | 887 | N | MET | A | 133 | 12135 | 21853 | 13119 | 3899 | 2110 | 497 | N |
| ATOM | 888 | CA | MET | A | 133 | 92.512 | −60.514 | 78.010 | 1.00 | 122.69 | | C |
| ANISOU | 888 | CA | MET | A | 133 | 11729 | 21883 | 13004 | 3405 | 1924 | −27 | C |
| ATOM | 889 | C | MET | A | 133 | 92.016 | −61.649 | 77.116 | 1.00 | 125.35 | | C |
| ANISOU | 889 | C | MET | A | 133 | 11500 | 23180 | 12947 | 3199 | 1800 | −608 | C |
| ATOM | 890 | CB | MET | A | 133 | 91.310 | −59.750 | 78.561 | 1.00 | 124.83 | | C |
| ANISOU | 890 | CB | MET | A | 133 | 12007 | 22263 | 13159 | 3664 | 1940 | 132 | C |
| ATOM | 891 | O | ALA | A | 134 | 92.329 | −64.588 | 74.521 | 1.00 | 123.20 | | O |
| ANISOU | 891 | O | ALA | A | 134 | 10406 | 24287 | 12115 | 2758 | 1788 | −1587 | O |
| ATOM | 892 | N | ALA | A | 134 | 91.777 | −61.377 | 75.838 | 1.00 | 122.62 | | N |
| ANISOU | 892 | N | ALA | A | 134 | 10984 | 23267 | 12338 | 3385 | 1825 | −608 | N |
| ATOM | 893 | CA | ALA | A | 134 | 91.450 | −62.429 | 74.864 | 1.00 | 122.31 | | C |
| ANISOU | 893 | CA | ALA | A | 134 | 10415 | 24122 | 11936 | 3169 | 1746 | −1188 | C |
| ATOM | 894 | C | ALA | A | 134 | 92.622 | −63.414 | 74.761 | 1.00 | 124.28 | | C |
| ANISOU | 894 | C | ALA | A | 134 | 10841 | 23820 | 12561 | 2715 | 1785 | −1367 | C |
| ATOM | 895 | CB | ALA | A | 134 | 91.174 | −61.812 | 73.517 | 1.00 | 123.10 | | C |
| ANISOU | 895 | CB | ALA | A | 134 | 10097 | 25394 | 11281 | 3887 | 1747 | −1023 | C |
| ATOM | 896 | N | GLU | A | 135 | 93.888 | −62.974 | 74.844 | 1.00 | 120.09 | | N |
| ANISOU | 896 | N | GLU | A | 135 | 10780 | 22177 | 12670 | 2308 | 1819 | −1269 | N |
| ATOM | 897 | CA | GLU | A | 135 | 94.956 | −63.981 | 74.638 | 1.00 | 119.66 | | C |
| ANISOU | 897 | CA | GLU | A | 135 | 10945 | 21469 | 13052 | 1857 | 1869 | −1413 | C |
| ATOM | 898 | C | GLU | A | 135 | 94.969 | −65.039 | 75.743 | 1.00 | 122.60 | | C |
| ANISOU | 898 | C | GLU | A | 135 | 11558 | 21078 | 13948 | 1295 | 1886 | −1631 | C |
| ATOM | 899 | O | GLU | A | 135 | 94.738 | −66.189 | 75.385 | 1.00 | 121.74 | | O |
| ANISOU | 899 | O | GLU | A | 135 | 11850 | 20100 | 14304 | 1127 | 1918 | −1413 | O |
| ATOM | 900 | CB | GLU | A | 135 | 96.302 | −63.278 | 74.518 | 1.00 | 121.04 | | C |
| ANISOU | 900 | CB | GLU | A | 135 | 11490 | 21093 | 13405 | 2165 | 1939 | −868 | C |
| ATOM | 901 | N | ASP | A | 136 | 95.210 | −64.665 | 77.006 | 1.00 | 118.82 | | N |
| ANISOU | 901 | N | ASP | A | 136 | 10793 | 21016 | 13337 | 1047 | 1881 | −2067 | N |
| ATOM | 902 | CA | ASP | A | 136 | 95.091 | −65.553 | 78.211 | 1.00 | 118.19 | | C |
| ANISOU | 902 | CA | ASP | A | 136 | 10835 | 20443 | 13628 | 543 | 1956 | −2361 | C |
| ATOM | 903 | C | ASP | A | 136 | 95.255 | −64.747 | 79.501 | 1.00 | 117.79 | | C |
| ANISOU | 903 | C | ASP | A | 136 | 11234 | 19598 | 13922 | 637 | 1908 | −1908 | C |
| ATOM | 904 | O | ASP | A | 136 | 95.886 | −63.711 | 79.482 | 1.00 | 118.50 | | O |
| ANISOU | 904 | O | ASP | A | 136 | 11514 | 19150 | 14361 | 268 | 1986 | −2047 | O |
| ATOM | 905 | CB | ASP | A | 136 | 95.902 | −66.857 | 78.269 | 1.00 | 120.49 | | C |
| ANISOU | 905 | CB | ASP | A | 136 | 11199 | 20322 | 14260 | 20 | 2132 | −2730 | C |
| ATOM | 906 | CG | ASP | A | 136 | 95.053 | −67.982 | 78.855 | 1.00 | 135.22 | | C |
| ANISOU | 906 | CG | ASP | A | 136 | 13067 | 21912 | 16400 | −520 | 2337 | −3189 | C |
| ATOM | 907 | OD1 | ASP | A | 136 | 94.641 | −68.866 | 78.100 | 1.00 | 137.12 | | O |
| ANISOU | 907 | OD1 | ASP | A | 136 | 12926 | 22788 | 16384 | −683 | 2390 | −3647 | O |
| ATOM | 908 | OD2 | ASP | A | 136 | 94.751 | −67.924 | 80.056 | 1.00 | 141.97 | | O |
| ANISOU | 908 | OD2 | ASP | A | 136 | 14299 | 21929 | 17713 | −757 | 2475 | −3085 | O |
| ATOM | 909 | N | VAL | A | 137 | 94.511 | −65.124 | 80.525 | 1.00 | 107.90 | | N |
| ANISOU | 909 | N | VAL | A | 137 | 10135 | 18313 | 12547 | 1142 | 1826 | −1389 | N |
| ATOM | 910 | CA | VAL | A | 137 | 94.714 | −64.348 | 81.768 | 1.00 | 103.75 | | C |
| ANISOU | 910 | CA | VAL | A | 137 | 9980 | 17153 | 12287 | 1254 | 1803 | −1008 | C |
| ATOM | 911 | C | VAL | A | 137 | 96.150 | −64.584 | 82.202 | 1.00 | 98.15 | | C |
| ANISOU | 911 | C | VAL | A | 137 | 9665 | 15562 | 12066 | 1017 | 1828 | −850 | C |
| ATOM | 912 | O | VAL | A | 137 | 96.582 | −63.888 | 83.097 | 1.00 | 97.05 | | O |
| ANISOU | 912 | O | VAL | A | 137 | 9807 | 14929 | 12140 | 1083 | 1812 | −580 | O |
| ATOM | 913 | CB | VAL | A | 137 | 93.733 | −64.766 | 82.873 | 1.00 | 107.19 | | C |
| ANISOU | 913 | CB | VAL | A | 137 | 10330 | 17710 | 12686 | 1132 | 1780 | −1186 | C |
| ATOM | 914 | N | ARG | A | 138 | 96.862 | −65.504 | 81.552 | 1.00 | 88.47 | | N |
| ANISOU | 914 | N | ARG | A | 138 | 8433 | 14188 | 10992 | 754 | 1878 | −1039 | N |
| ATOM | 915 | CA | ARG | A | 138 | 98.259 | −65.837 | 81.910 | 1.00 | 85.58 | | C |
| ANISOU | 915 | CA | ARG | A | 138 | 8376 | 13109 | 11034 | 571 | 1906 | −910 | C |
| ATOM | 916 | C | ARG | A | 138 | 99.135 | −64.619 | 81.688 | 1.00 | 82.06 | | C |
| ANISOU | 916 | C | ARG | A | 138 | 8142 | 12376 | 10662 | 880 | 1885 | −480 | C |
| ATOM | 917 | O | ARG | A | 138 | 100.017 | −64.388 | 82.496 | 1.00 | 80.36 | | O |
| ANISOU | 917 | O | ARG | A | 138 | 8179 | 11605 | 10750 | 802 | 1876 | −321 | O |
| ATOM | 918 | CB | ARG | A | 138 | 98.756 | −66.973 | 81.028 | 1.00 | 86.06 | | C |
| ANISOU | 918 | CB | ARG | A | 138 | 8346 | 13168 | 11183 | 293 | 2008 | −1198 | C |
| ATOM | 919 | CG | ARG | A | 138 | 97.813 | −68.157 | 80.976 | 1.00 | 91.75 | | C |
| ANISOU | 919 | CG | ARG | A | 138 | 8720 | 14457 | 11683 | 52 | 2106 | −1706 | C |
| ATOM | 920 | CD | ARG | A | 138 | 98.171 | −69.016 | 79.796 | 1.00 | 94.11 | | C |
| ANISOU | 920 | CD | ARG | A | 138 | 8896 | 14858 | 12004 | −157 | 2236 | −1988 | C |
| ATOM | 921 | NE | ARG | A | 138 | 98.925 | −70.123 | 80.337 | 1.00 | 99.94 | | N |
| ANISOU | 921 | NE | ARG | A | 138 | 9854 | 14977 | 13141 | −511 | 2434 | −2123 | N |
| ATOM | 922 | CZ | ARG | A | 138 | 99.196 | −71.216 | 79.660 | 1.00 | 116.10 | | C |
| ANISOU | 922 | CZ | ARG | A | 138 | 11818 | 17005 | 15290 | −829 | 2672 | −2499 | C |
| ATOM | 923 | NH1 | ARG | A | 138 | 98.778 | −71.331 | 78.415 | 1.00 | 104.53 | | N |
| ANISOU | 923 | NH1 | ARG | A | 138 | 10008 | 16172 | 13538 | −889 | 2706 | −2847 | N |
| ATOM | 924 | NH2 | ARG | A | 138 | 99.880 | −72.188 | 80.227 | 1.00 | 104.52 | | N |
| ANISOU | 924 | NH2 | ARG | A | 138 | 10612 | 14904 | 14196 | −1064 | 2911 | −2527 | N |
| ATOM | 925 | N | ILE | A | 139 | 98.867 | −63.874 | 80.623 | 1.00 | 74.71 | | N |
| ANISOU | 925 | N | ILE | A | 139 | 7094 | 11858 | 9435 | 1243 | 1914 | −309 | N |
| ATOM | 926 | CA | ILE | A | 139 | 99.660 | −62.653 | 80.361 | 1.00 | 73.64 | | C |
| ANISOU | 926 | CA | ILE | A | 139 | 7164 | 11468 | 9347 | 1570 | 2006 | 97 | C |
| ATOM | 927 | C | ILE | A | 139 | 99.473 | −61.715 | 81.535 | 1.00 | 77.72 | | C |
| ANISOU | 927 | C | ILE | A | 139 | 7904 | 11610 | 10016 | 1647 | 2033 | 298 | C |
| ATOM | 928 | O | ILE | A | 139 | 100.448 | −61.189 | 82.010 | 1.00 | 77.33 | | O |
| ANISOU | 928 | O | ILE | A | 139 | 8100 | 11024 | 10259 | 1602 | 2106 | 461 | O |
| ATOM | 929 | CB | ILE | A | 139 | 99.088 | −61.930 | 79.155 | 1.00 | 76.71 | | C |
| ANISOU | 929 | CB | ILE | A | 139 | 7369 | 12468 | 9310 | 2026 | 2093 | 262 | C |
| ATOM | 930 | CG1 | ILE | A | 139 | 99.473 | −62.581 | 77.836 | 1.00 | 77.87 | | C |
| ANISOU | 930 | CG1 | ILE | A | 139 | 7337 | 12903 | 9348 | 1947 | 2090 | 87 | C |
| ATOM | 931 | CG2 | ILE | A | 139 | 99.530 | −60.494 | 79.199 | 1.00 | 77.25 | | C |
| ANISOU | 931 | CG2 | ILE | A | 139 | 7695 | 12245 | 9411 | 2442 | 2306 | 733 | C |
| ATOM | 932 | CD1 | ILE | A | 139 | 99.685 | −61.579 | 76.738 | 1.00 | 88.18 | | C |
| ANISOU | 932 | CD1 | ILE | A | 139 | 8406 | 14927 | 10171 | 2394 | 2155 | 201 | C |
| ATOM | 933 | N | ILE | A | 140 | 98.244 | −61.534 | 81.995 | 1.00 | 74.43 | | N |
| ANISOU | 933 | N | ILE | A | 140 | 7372 | 11515 | 9393 | 1742 | 1989 | 242 | N |
| ATOM | 934 | CA | ILE | A | 140 | 98.013 | −60.585 | 83.111 | 1.00 | 73.87 | | C |
| ANISOU | 934 | CA | ILE | A | 140 | 7483 | 11158 | 9426 | 1816 | 2024 | 394 | C |
| ATOM | 935 | C | ILE | A | 140 | 98.820 | −61.017 | 84.326 | 1.00 | 77.16 | | C |
| ANISOU | 935 | C | ILE | A | 140 | 8090 | 11006 | 10221 | 1427 | 1942 | 276 | C |
| ATOM | 936 | O | ILE | A | 140 | 99.606 | −60.190 | 84.752 | 1.00 | 77.95 | | O |
| ANISOU | 936 | O | ILE | A | 140 | 8408 | 10690 | 10520 | 1453 | 2019 | 433 | O |
| ATOM | 937 | CB | ILE | A | 140 | 96.526 | −60.442 | 83.441 | 1.00 | 77.00 | | C |
| ANISOU | 937 | CB | ILE | A | 140 | 7674 | 12093 | 9490 | 2009 | 1987 | 337 | C |
| ATOM | 938 | CG1 | ILE | A | 140 | 95.787 | −59.590 | 82.415 | 1.00 | 77.27 | | C |
| ANISOU | 938 | CG1 | ILE | A | 140 | 7491 | 12794 | 9074 | 2511 | 2079 | 509 | C |
| ATOM | 939 | CG2 | ILE | A | 140 | 96.381 | −59.845 | 84.815 | 1.00 | 77.58 | | C |
| ANISOU | 939 | CG2 | ILE | A | 140 | 7949 | 11838 | 9688 | 2068 | 2035 | 482 | C |
| ATOM | 940 | CD1 | ILE | A | 140 | 94.299 | −59.761 | 82.473 | 1.00 | 83.12 | | C |
| ANISOU | 940 | CD1 | ILE | A | 140 | 7831 | 14363 | 9390 | 2601 | 1971 | 256 | C |
| ATOM | 941 | N | ILE | A | 141 | 98.607 | −62.242 | 84.816 | 1.00 | 71.30 | | N |
| ANISOU | 941 | N | ILE | A | 141 | 7266 | 10254 | 9570 | 1091 | 1835 | 0 | N |
| ATOM | 942 | CA | ILE | A | 141 | 99.266 | −62.785 | 86.025 | 1.00 | 69.49 | | C |
| ANISOU | 942 | CA | ILE | A | 141 | 7202 | 9568 | 9632 | 816 | 1781 | −66 | C |
| ATOM | 943 | C | ILE | A | 141 | 100.707 | −62.327 | 86.136 | 1.00 | 69.56 | | C |
| ANISOU | 943 | C | ILE | A | 141 | 7376 | 9176 | 9878 | 804 | 1807 | 80 | C |
| ATOM | 944 | O | ILE | A | 141 | 101.147 | −61.865 | 87.189 | 1.00 | 69.36 | | O |
| ANISOU | 944 | O | ILE | A | 141 | 7487 | 8854 | 10012 | 748 | 1791 | 128 | O |
| ATOM | 945 | CB | ILE | A | 141 | 99.139 | −64.320 | 86.144 | 1.00 | 72.40 | | C |
| ANISOU | 945 | CB | ILE | A | 141 | 7489 | 9969 | 10053 | 530 | 1773 | −334 | C |
| ATOM | 946 | CG1 | ILE | A | 141 | 97.668 | −64.728 | 86.328 | 1.00 | 73.42 | | C |
| ANISOU | 946 | CG1 | ILE | A | 141 | 7452 | 10452 | 9995 | 461 | 1789 | −551 | C |
| ATOM | 947 | CG2 | ILE | A | 141 | 99.974 | −64.835 | 87.308 | 1.00 | 72.05 | | C |
| ANISOU | 947 | CG2 | ILE | A | 141 | 7630 | 9482 | 10265 | 372 | 1756 | −305 | C |
| ATOM | 948 | CD1 | ILE | A | 141 | 97.250 | −65.909 | 85.503 | 1.00 | 84.73 | | C |
| ANISOU | 948 | CD1 | ILE | A | 141 | 8663 | 12194 | 11335 | 254 | 1879 | −887 | C |
| ATOM | 949 | N | VAL | A | 142 | 101.430 | −62.428 | 85.042 | 1.00 | 63.67 | | N |
| ANISOU | 949 | N | VAL | A | 142 | 6586 | 8470 | 9135 | 850 | 1857 | 117 | N |
| ATOM | 950 | CA | VAL | A | 142 | 102.814 | −61.987 | 85.007 | 1.00 | 62.30 | | C |
| ANISOU | 950 | CA | VAL | A | 142 | 6529 | 7963 | 9181 | 826 | 1910 | 220 | C |
| ATOM | 951 | C | VAL | A | 142 | 102.877 | −60.477 | 85.242 | 1.00 | 65.10 | | C |
| ANISOU | 951 | C | VAL | A | 142 | 7018 | 8131 | 9585 | 990 | 2058 | 397 | C |
| ATOM | 952 | O | VAL | A | 142 | 103.647 | −60.051 | 86.098 | 1.00 | 65.01 | | O |
| ANISOU | 952 | O | VAL | A | 142 | 7104 | 7813 | 9783 | 859 | 2078 | 367 | O |
| ATOM | 953 | CB | VAL | A | 142 | 103.509 | −62.456 | 83.723 | 1.00 | 65.29 | | C |
| ANISOU | 953 | CB | VAL | A | 142 | 6828 | 8436 | 9543 | 842 | 1954 | 216 | C |
| ATOM | 954 | CG1 | VAL | A | 142 | 104.875 | −61.824 | 83.583 | 1.00 | 65.52 | | C |
| ANISOU | 954 | CG1 | VAL | A | 142 | 6959 | 8150 | 9785 | 835 | 2048 | 321 | C |
| ATOM | 955 | CG2 | VAL | A | 142 | 103.617 | −63.976 | 83.705 | 1.00 | 64.79 | | C |
| ANISOU | 955 | CG2 | VAL | A | 142 | 6680 | 8425 | 9515 | 625 | 1881 | 10 | C |
| ATOM | 956 | N | LYS | A | 143 | 101.988 | −59.693 | 84.581 | 1.00 | 59.81 | | N |
| ANISOU | 956 | N | LYS | A | 143 | 6341 | 7681 | 8704 | 1285 | 2188 | 558 | N |
| ATOM | 957 | CA | LYS | A | 143 | 101.907 | −58.233 | 84.737 | 1.00 | 58.47 | | C |
| ANISOU | 957 | CA | LYS | A | 143 | 6343 | 7291 | 8583 | 1496 | 2439 | 763 | C |
| ATOM | 958 | C | LYS | A | 143 | 101.545 | −57.816 | 86.175 | 1.00 | 58.38 | | C |
| ANISOU | 958 | C | LYS | A | 143 | 6429 | 7067 | 8686 | 1367 | 2414 | 685 | C |
| ATOM | 959 | O | LYS | A | 143 | 101.941 | −56.744 | 86.620 | 1.00 | 56.40 | | O |
| ANISOU | 959 | O | LYS | A | 143 | 6342 | 6474 | 8614 | 1369 | 2640 | 738 | O |
| ATOM | 960 | CB | LYS | A | 143 | 100.938 | −57.639 | 83.726 | 1.00 | 61.34 | | C |
| ANISOU | 960 | CB | LYS | A | 143 | 6662 | 8014 | 8630 | 1931 | 2596 | 998 | C |
| ATOM | 961 | CG | LYS | A | 143 | 101.381 | −56.305 | 83.165 | 1.00 | 81.30 | | C |
| ANISOU | 961 | CG | LYS | A | 143 | 9397 | 10264 | 11228 | 2225 | 2993 | 1293 | C |
| ATOM | 962 | CD | LYS | A | 143 | 100.220 | −55.339 | 82.998 | 1.00 | 100.20 | | C |
| ANISOU | 962 | CD | LYS | A | 143 | 11848 | 12851 | 13371 | 2691 | 3219 | 1572 | C |
| ATOM | 963 | CE | LYS | A | 143 | 99.045 | −55.853 | 82.176 | 1.00 | 119.41 | | C |
| ANISOU | 963 | CE | LYS | A | 143 | 14004 | 16040 | 15326 | 3018 | 3056 | 1621 | C |
| ATOM | 964 | NZ | LYS | A | 143 | 97.946 | −54.857 | 82.016 | 1.00 | 125.42 | | N |
| ANISOU | 964 | NZ | LYS | A | 143 | 14801 | 17054 | 15800 | 3558 | 3293 | 1934 | N |
| ATOM | 965 | N | LEU | A | 144 | 100.828 | −58.685 | 86.902 | 1.00 | 53.42 | | N |
| ANISOU | 965 | N | LEU | A | 144 | 5702 | 6626 | 7971 | 1231 | 2179 | 529 | N |
| ATOM | 966 | CA | LEU | A | 144 | 100.480 | −58.490 | 88.305 | 1.00 | 52.10 | | C |
| ANISOU | 966 | CA | LEU | A | 144 | 5605 | 6309 | 7882 | 1100 | 2118 | 439 | C |
| ATOM | 967 | C | LEU | A | 144 | 101.719 | −58.715 | 89.170 | 1.00 | 53.86 | | C |
| ANISOU | 967 | C | LEU | A | 144 | 5869 | 6252 | 8343 | 824 | 2047 | 291 | C |
| ATOM | 968 | O | LEU | A | 144 | 101.965 | −57.942 | 90.096 | 1.00 | 52.20 | | O |
| ANISOU | 968 | O | LEU | A | 144 | 5746 | 5834 | 8255 | 741 | 2124 | 229 | O |
| ATOM | 969 | CB | LEU | A | 144 | 99.374 | −59.461 | 88.721 | 1.00 | 52.24 | | C |
| ANISOU | 969 | CB | LEU | A | 144 | 5500 | 6619 | 7728 | 1043 | 1932 | 320 | C |
| ATOM | 970 | CG | LEU | A | 144 | 97.970 | −59.004 | 88.418 | 1.00 | 57.48 | | C |
| ANISOU | 970 | CG | LEU | A | 144 | 6092 | 7610 | 8139 | 1298 | 1987 | 405 | C |
| ATOM | 971 | CD1 | LEU | A | 144 | 97.063 | −60.183 | 88.210 | 1.00 | 58.91 | | C |
| ANISOU | 971 | CD1 | LEU | A | 144 | 6061 | 8191 | 8132 | 1207 | 1846 | 216 | C |
| ATOM | 972 | CD2 | LEU | A | 144 | 97.433 | −58.159 | 89.538 | 1.00 | 57.12 | | C |
| ANISOU | 972 | CD2 | LEU | A | 144 | 6174 | 7393 | 8136 | 1336 | 2051 | 445 | C |
| ATOM | 973 | N | ALA | A | 145 | 102.514 | −59.759 | 88.855 | 1.00 | 50.11 | | N |
| ANISOU | 973 | N | ALA | A | 145 | 5309 | 5815 | 7914 | 697 | 1921 | 216 | N |
| ATOM | 974 | CA | ALA | A | 145 | 103.734 | −60.074 | 89.590 | 1.00 | 49.81 | | C |
| ANISOU | 974 | CA | ALA | A | 145 | 5255 | 5631 | 8039 | 508 | 1841 | 94 | C |
| ATOM | 975 | C | ALA | A | 145 | 104.754 | −58.958 | 89.382 | 1.00 | 55.44 | | C |
| ANISOU | 975 | C | ALA | A | 145 | 6010 | 6124 | 8930 | 462 | 2032 | 71 | C |
| ATOM | 976 | O | ALA | A | 145 | 105.359 | −58.470 | 90.345 | 1.00 | 56.16 | | O |
| ANISOU | 976 | O | ALA | A | 145 | 6091 | 6116 | 9132 | 311 | 2049 | −86 | O |
| ATOM | 977 | CB | ALA | A | 145 | 104.293 | −61.379 | 89.090 | 1.00 | 50.37 | | C |
| ANISOU | 977 | CB | ALA | A | 145 | 5240 | 5787 | 8109 | 457 | 1735 | 71 | C |
| ATOM | 978 | N | ASP | A | 146 | 104.895 | −58.526 | 88.116 | 1.00 | 51.29 | | N |
| ANISOU | 978 | N | ASP | A | 146 | 5521 | 5548 | 8418 | 591 | 2214 | 206 | N |
| ATOM | 979 | CA | ASP | A | 146 | 105.756 | −57.441 | 87.680 | 1.00 | 51.01 | | C |
| ANISOU | 979 | CA | ASP | A | 146 | 5562 | 5250 | 8568 | 570 | 2506 | 215 | C |
| ATOM | 980 | C | ASP | A | 146 | 105.364 | −56.159 | 88.403 | 1.00 | 54.53 | | C |
| ANISOU | 980 | C | ASP | A | 146 | 6145 | 5469 | 9104 | 563 | 2756 | 185 | C |
| ATOM | 981 | O | ASP | A | 146 | 106.250 | −55.504 | 88.951 | 1.00 | 54.92 | | O |
| ANISOU | 981 | O | ASP | A | 146 | 6197 | 5305 | 9363 | 340 | 2919 | −22 | O |
| ATOM | 982 | CB | ASP | A | 146 | 105.621 | −57.278 | 86.164 | 1.00 | 53.66 | | C |
| ANISOU | 982 | CB | ASP | A | 146 | 5941 | 5626 | 8821 | 813 | 2679 | 444 | C |
| ATOM | 983 | CG | ASP | A | 146 | 106.322 | −56.082 | 85.575 | 1.00 | 76.48 | | C |
| ANISOU | 983 | CG | ASP | A | 146 | 8969 | 8194 | 11896 | 860 | 3091 | 524 | C |
| ATOM | 984 | OD2 | ASP | A | 146 | 107.442 | −56.254 | 85.047 | 1.00 | 82.21 | | O |
| ANISOU | 984 | OD2 | ASP | A | 146 | 9647 | 8831 | 12759 | 741 | 3145 | 461 | O |
| ATOM | 985 | OD1 | ASP | A | 146 | 105.740 | −54.974 | 85.618 | 1.00 | 82.17 | | O |
| ANISOU | 985 | OD1 | ASP | A | 146 | 9859 | 8730 | 12632 | 1030 | 3408 | 660 | O |
| ATOM | 986 | N | ARG | A | 147 | 104.036 | −55.817 | 88.424 | 1.00 | 49.30 | | N |
| ANISOU | 986 | N | ARG | A | 147 | 5572 | 4882 | 8278 | 795 | 2806 | 355 | N |
| ATOM | 987 | CA | ARG | A | 147 | 103.493 | −54.607 | 89.060 | 1.00 | 47.66 | | C |
| ANISOU | 987 | CA | ARG | A | 147 | 5524 | 4444 | 8143 | 845 | 3088 | 368 | C |
| ATOM | 988 | C | ARG | A | 147 | 103.820 | −54.581 | 90.527 | 1.00 | 51.02 | | C |
| ANISOU | 988 | C | ARG | A | 147 | 5896 | 4811 | 8676 | 534 | 2967 | 62 | C |
| ATOM | 989 | O | ARG | A | 147 | 104.282 | −53.548 | 91.012 | 1.00 | 49.53 | | O |
| ANISOU | 989 | O | ARG | A | 147 | 5792 | 4335 | 8693 | 375 | 3272 | −104 | O |
| ATOM | 990 | CB | ARG | A | 147 | 101.972 | −54.444 | 88.817 | 1.00 | 43.62 | | C |
| ANISOU | 990 | CB | ARG | A | 147 | 5063 | 4132 | 7379 | 1193 | 3103 | 615 | C |
| ATOM | 991 | CG | ARG | A | 147 | 101.320 | −53.199 | 89.433 | 1.00 | 42.77 | | C |
| ANISOU | 991 | CG | ARG | A | 147 | 5143 | 3774 | 7334 | 1307 | 3433 | 676 | C |
| ATOM | 992 | CD | ARG | A | 147 | 101.997 | −51.919 | 89.010 | 1.00 | 54.45 | | C |
| ANISOU | 992 | CD | ARG | A | 147 | 6837 | 4791 | 9060 | 1352 | 3985 | 749 | C |
| ATOM | 993 | NE | ARG | A | 147 | 101.774 | −51.642 | 87.590 | 1.00 | 68.33 | | N |
| ANISOU | 993 | NE | ARG | A | 147 | 8675 | 6608 | 10680 | 1779 | 4230 | 1121 | N |
| ATOM | 994 | CZ | ARG | A | 147 | 100.922 | −50.731 | 87.127 | 1.00 | 77.98 | | C |
| ANISOU | 994 | CZ | ARG | A | 147 | 10075 | 7773 | 11782 | 2237 | 4615 | 1465 | C |
| ATOM | 995 | NH1 | ARG | A | 147 | 100.207 | −49.996 | 87.967 | 1.00 | 61.55 | | N |
| ANISOU | 995 | NH1 | ARG | A | 147 | 8128 | 5516 | 9741 | 2296 | 4812 | 1471 | N |
| ATOM | 996 | NH2 | ARG | A | 147 | 100.783 | −50.548 | 85.818 | 1.00 | 58.95 | | N |
| ANISOU | 996 | NH2 | ARG | A | 147 | 7705 | 5508 | 9184 | 2679 | 4823 | 1822 | N |
| ATOM | 997 | N | LEU | A | 148 | 103.625 | −55.729 | 91.220 | 1.00 | 48.76 | | N |
| ANISOU | 997 | N | LEU | A | 148 | 5466 | 4809 | 8251 | 446 | 2566 | −31 | N |
| ATOM | 998 | CA | LEU | A | 148 | 103.915 | −55.870 | 92.649 | 1.00 | 48.54 | | C |
| ANISOU | 998 | CA | LEU | A | 148 | 5357 | 4845 | 8243 | 221 | 2406 | −291 | C |
| ATOM | 999 | C | LEU | A | 148 | 105.394 | −55.582 | 92.921 | 1.00 | 52.39 | | C |
| ANISOU | 999 | C | LEU | A | 148 | 5727 | 5269 | 8908 | −41 | 2478 | −568 | C |
| ATOM | 1000 | O | LEU | A | 148 | 105.710 | −54.825 | 93.849 | 1.00 | 52.04 | | O |
| ANISOU | 1000 | O | LEU | A | 148 | 5652 | 5164 | 8955 | −243 | 2603 | −842 | O |
| ATOM | 1001 | CB | LEU | A | 148 | 103.509 | −57.254 | 93.178 | 1.00 | 48.17 | | C |
| ANISOU | 1001 | CB | LEU | A | 148 | 5206 | 5089 | 8006 | 246 | 2040 | −268 | C |
| ATOM | 1002 | CG | LEU | A | 148 | 103.964 | −57.580 | 94.606 | 1.00 | 52.30 | | C |
| ANISOU | 1002 | CG | LEU | A | 148 | 5622 | 5763 | 8487 | 94 | 1860 | −483 | C |
| ATOM | 1003 | CD1 | LEU | A | 148 | 103.301 | −56.671 | 95.642 | 1.00 | 52.64 | | C |
| ANISOU | 1003 | CD1 | LEU | A | 148 | 5734 | 5747 | 8521 | 40 | 1958 | −602 | C |
| ATOM | 1004 | CD2 | LEU | A | 148 | 103.738 | −59.014 | 94.931 | 1.00 | 53.55 | | C |
| ANISOU | 1004 | CD2 | LEU | A | 148 | 5725 | 6136 | 8487 | 174 | 1605 | −393 | C |
| ATOM | 1005 | N | HIS | A | 149 | 106.287 | −56.144 | 92.084 | 1.00 | 48.33 | | N |
| ANISOU | 1005 | N | HIS | A | 149 | 5126 | 4800 | 8438 | −51 | 2424 | −535 | N |
| ATOM | 1006 | CA | HIS | A | 149 | 107.704 | −55.884 | 92.247 | 1.00 | 48.38 | | C |
| ANISOU | 1006 | CA | HIS | A | 149 | 4978 | 4804 | 8600 | −294 | 2499 | −817 | C |
| ATOM | 1007 | C | HIS | A | 149 | 108.058 | −54.418 | 91.988 | 1.00 | 56.44 | | C |
| ANISOU | 1007 | C | HIS | A | 149 | 6110 | 5460 | 9876 | −448 | 2981 | −967 | C |
| ATOM | 1008 | O | HIS | A | 149 | 108.837 | −53.839 | 92.747 | 1.00 | 57.08 | | O |
| ANISOU | 1008 | O | HIS | A | 149 | 6056 | 5551 | 10081 | −744 | 3107 | −1355 | O |
| ATOM | 1009 | CB | HIS | A | 149 | 108.563 | −56.807 | 91.403 | 1.00 | 48.10 | | C |
| ANISOU | 1009 | CB | HIS | A | 149 | 4830 | 4888 | 8559 | −255 | 2357 | −738 | C |
| ATOM | 1010 | CG | HIS | A | 149 | 110.010 | −56.634 | 91.710 | 1.00 | 50.76 | | C |
| ANISOU | 1010 | CG | HIS | A | 149 | 4945 | 5329 | 9012 | −496 | 2389 | −1060 | C |
| ATOM | 1011 | ND1 | HIS | A | 149 | 110.930 | −56.406 | 90.718 | 1.00 | 52.65 | | N |
| ANISOU | 1011 | ND1 | HIS | A | 149 | 5156 | 5429 | 9420 | −568 | 2583 | −1085 | N |
| ATOM | 1012 | CD2 | HIS | A | 149 | 110.636 | −56.598 | 92.908 | 1.00 | 51.89 | | C |
| ANISOU | 1012 | CD2 | HIS | A | 149 | 4858 | 5756 | 9101 | −675 | 2268 | −1395 | C |
| ATOM | 1013 | CE1 | HIS | A | 149 | 112.088 | −56.235 | 91.335 | 1.00 | 51.81 | | C |
| ANISOU | 1013 | CE1 | HIS | A | 149 | 4792 | 5518 | 9376 | −815 | 2580 | −1458 | C |
| ATOM | 1014 | NE2 | HIS | A | 149 | 111.960 | −56.346 | 92.654 | 1.00 | 51.67 | | N |
| ANISOU | 1014 | NE2 | HIS | A | 149 | 4626 | 5801 | 9206 | −877 | 2381 | −1663 | N |
| ATOM | 1015 | N | ASN | A | 150 | 107.461 | −53.809 | 90.948 | 1.00 | 54.36 | | N |
| ANISOU | 1015 | N | ASN | A | 150 | 6081 | 4898 | 9674 | −236 | 3294 | −677 | N |
| ATOM | 1016 | CA | ASN | A | 150 | 107.678 | −52.398 | 90.645 | 1.00 | 55.00 | | C |
| ANISOU | 1016 | CA | ASN | A | 150 | 6346 | 4537 | 10015 | −312 | 3873 | −741 | C |
| ATOM | 1017 | C | ASN | A | 150 | 107.268 | −51.536 | 91.875 | 1.00 | 59.11 | | C |
| ANISOU | 1017 | C | ASN | A | 150 | 6916 | 4930 | 10614 | −495 | 4059 | −1006 | C |
| ATOM | 1018 | O | ASN | A | 150 | 108.051 | −50.706 | 92.330 | 1.00 | 60.29 | | O |
| ANISOU | 1018 | O | ASN | A | 150 | 7023 | 4883 | 11003 | −835 | 4397 | −1397 | O |
| ATOM | 1019 | CB | ASN | A | 150 | 106.880 | −51.977 | 89.398 | 1.00 | 57.49 | | C |
| ANISOU | 1019 | CB | ASN | A | 150 | 6921 | 4638 | 10286 | 93 | 4166 | −280 | C |
| ATOM | 1020 | CG | ASN | A | 150 | 107.470 | −52.299 | 88.046 | 1.00 | 78.60 | | C |
| ANISOU | 1020 | CG | ASN | A | 150 | 9597 | 7303 | 12966 | 233 | 4229 | −75 | C |
| ATOM | 1021 | OD1 | ASN | A | 150 | 108.671 | −52.590 | 87.878 | 1.00 | 73.12 | | O |
| ANISOU | 1021 | OD1 | ASN | A | 150 | 8755 | 6621 | 12408 | −12 | 4192 | −289 | O |
| ATOM | 1022 | ND2 | ASN | A | 150 | 106.608 | −52.225 | 87.038 | 1.00 | 69.19 | | N |
| ANISOU | 1022 | ND2 | ASN | A | 150 | 8553 | 6140 | 11596 | 661 | 4335 | 344 | N |
| ATOM | 1023 | N | LEU | A | 151 | 106.087 | −51.779 | 92.443 | 1.00 | 54.00 | | N |
| ANISOU | 1023 | N | LEU | A | 151 | 6327 | 4426 | 9766 | −310 | 3841 | −850 | N |
| ATOM | 1024 | CA | LEU | A | 151 | 105.623 | −51.030 | 93.614 | 1.00 | 53.79 | | C |
| ANISOU | 1024 | CA | LEU | A | 151 | 6350 | 4302 | 9788 | −462 | 3993 | −1086 | C |
| ATOM | 1025 | C | LEU | A | 151 | 106.579 | −51.153 | 94.809 | 1.00 | 60.75 | | C |
| ANISOU | 1025 | C | LEU | A | 151 | 6947 | 5438 | 10696 | −876 | 3823 | −1618 | C |
| ATOM | 1026 | O | LEU | A | 151 | 106.789 | −50.168 | 95.539 | 1.00 | 60.15 | | O |
| ANISOU | 1026 | O | LEU | A | 151 | 6876 | 5187 | 10790 | −1159 | 4163 | −1994 | O |
| ATOM | 1027 | CB | LEU | A | 151 | 104.208 | −51.458 | 94.008 | 1.00 | 52.80 | | C |
| ANISOU | 1027 | CB | LEU | A | 151 | 6294 | 4360 | 9409 | −184 | 3722 | −823 | C |
| ATOM | 1028 | CG | LEU | A | 151 | 103.103 | −51.082 | 93.026 | 1.00 | 55.97 | | C |
| ANISOU | 1028 | CG | LEU | A | 151 | 6929 | 4602 | 9734 | 245 | 3947 | −364 | C |
| ATOM | 1029 | CD1 | LEU | A | 151 | 101.997 | −52.069 | 93.075 | 1.00 | 55.59 | | C |
| ANISOU | 1029 | CD1 | LEU | A | 151 | 6809 | 4934 | 9379 | 486 | 3515 | −140 | C |
| ATOM | 1030 | CD2 | LEU | A | 151 | 102.588 | −49.696 | 93.269 | 1.00 | 56.76 | | C |
| ANISOU | 1030 | CD2 | LEU | A | 151 | 7281 | 4288 | 9996 | 312 | 4499 | −344 | C |
| ATOM | 1031 | N | ARG | A | 152 | 107.186 | −52.358 | 94.968 | 1.00 | 58.76 | | N |
| ANISOU | 1031 | N | ARG | A | 152 | 6435 | 5628 | 10263 | −891 | 3337 | −1660 | N |
| ATOM | 1032 | CA | ARG | A | 152 | 108.151 | −52.681 | 96.021 | 1.00 | 59.05 | | C |
| ANISOU | 1032 | CA | ARG | A | 152 | 6137 | 6080 | 10218 | −1167 | 3103 | −2102 | C |
| ATOM | 1033 | C | ARG | A | 152 | 109.460 | −51.915 | 95.834 | 1.00 | 67.71 | | C |
| ANISOU | 1033 | C | ARG | A | 152 | 7075 | 7085 | 11568 | −1533 | 3448 | −2541 | C |
| ATOM | 1034 | O | ARG | A | 152 | 110.253 | −51.786 | 96.760 | 1.00 | 67.89 | | O |
| ANISOU | 1034 | O | ARG | A | 152 | 6786 | 7462 | 11548 | −1828 | 3388 | −3037 | O |
| ATOM | 1035 | CB | ARG | A | 152 | 108.430 | −54.184 | 96.035 | 1.00 | 54.64 | | C |
| ANISOU | 1035 | CB | ARG | A | 152 | 5398 | 5962 | 9402 | −976 | 2587 | −1917 | C |
| ATOM | 1036 | CG | ARG | A | 152 | 107.342 | −55.005 | 96.687 | 1.00 | 54.09 | | C |
| ANISOU | 1036 | CG | ARG | A | 152 | 5392 | 6087 | 9073 | −734 | 2260 | −1668 | C |
| ATOM | 1037 | CD | ARG | A | 152 | 107.459 | −56.458 | 96.287 | 1.00 | 58.34 | | C |
| ANISOU | 1037 | CD | ARG | A | 152 | 5874 | 6849 | 9444 | −504 | 1926 | −1383 | C |
| ATOM | 1038 | NE | ARG | A | 152 | 106.667 | −57.335 | 97.152 | 1.00 | 60.75 | | N |
| ANISOU | 1038 | NE | ARG | A | 152 | 6195 | 7382 | 9507 | −326 | 1658 | −1235 | N |
| ATOM | 1039 | CZ | ARG | A | 152 | 106.757 | −58.664 | 97.185 | 1.00 | 73.98 | | C |
| ANISOU | 1039 | CZ | ARG | A | 152 | 7823 | 9264 | 11021 | −135 | 1422 | −1035 | C |
| ATOM | 1040 | NH1 | ARG | A | 152 | 107.614 | −59.299 | 96.399 | 1.00 | 62.96 | | N |
| ANISOU | 1040 | NH1 | ARG | A | 152 | 6348 | 7905 | 9668 | −90 | 1381 | −961 | N |
| ATOM | 1041 | NH2 | ARG | A | 152 | 105.990 | −59.367 | 98.007 | 1.00 | 62.72 | | N |
| ANISOU | 1041 | NH2 | ARG | A | 152 | 6449 | 7975 | 9405 | 17 | 1275 | −905 | N |
| ATOM | 1042 | N | THR | A | 153 | 109.714 | −51.467 | 94.622 | 1.00 | 67.96 | | N |
| ANISOU | 1042 | N | THR | A | 153 | 7286 | 6703 | 11833 | −1503 | 3807 | −2374 | N |
| ATOM | 1043 | CA | THR | A | 153 | 110.922 | −50.727 | 94.280 | 1.00 | 69.76 | | C |
| ANISOU | 1043 | CA | THR | A | 153 | 7403 | 6757 | 12347 | −1857 | 4222 | −2768 | C |
| ATOM | 1044 | C | THR | A | 153 | 110.546 | −49.348 | 93.691 | 1.00 | 80.29 | | C |
| ANISOU | 1044 | C | THR | A | 153 | 9104 | 7384 | 14020 | −1902 | 4964 | −2712 | C |
| ATOM | 1045 | O | THR | A | 153 | 111.229 | −48.876 | 92.782 | 1.00 | 81.19 | | O |
| ANISOU | 1045 | O | THR | A | 153 | 9301 | 7164 | 14386 | −1992 | 5384 | −2727 | O |
| ATOM | 1046 | CB | THR | A | 153 | 111.739 | −51.562 | 93.302 | 1.00 | 73.53 | | C |
| ANISOU | 1046 | CB | THR | A | 153 | 7763 | 7378 | 12798 | −1750 | 4019 | −2590 | C |
| ATOM | 1047 | OG1 | THR | A | 153 | 110.996 | −51.736 | 92.086 | 1.00 | 69.86 | | O |
| ANISOU | 1047 | OG1 | THR | A | 153 | 7619 | 6592 | 12332 | −1371 | 4092 | −2016 | O |
| ATOM | 1048 | CG2 | THR | A | 153 | 112.147 | −52.899 | 93.888 | 1.00 | 73.30 | | C |
| ANISOU | 1048 | CG2 | THR | A | 153 | 7407 | 7994 | 12451 | −1658 | 3387 | −2613 | C |
| ATOM | 1049 | N | LEU | A | 154 | 109.451 | −48.718 | 94.187 | 1.00 | 80.09 | | N |
| ANISOU | 1049 | N | LEU | A | 154 | 9318 | 7116 | 13998 | −1798 | 5163 | −2612 | N |
| ATOM | 1050 | CA | LEU | A | 154 | 108.956 | −47.439 | 93.670 | 1.00 | 81.38 | | C |
| ANISOU | 1050 | CA | LEU | A | 154 | 9878 | 6593 | 14452 | −1729 | 5912 | −2465 | C |
| ATOM | 1051 | C | LEU | A | 154 | 109.692 | −46.215 | 94.237 | 1.00 | 93.59 | | C |
| ANISOU | 1051 | C | LEU | A | 154 | 11398 | 7794 | 16368 | −2267 | 6579 | −3109 | C |
| ATOM | 1052 | O | LEU | A | 154 | 109.441 | −45.103 | 93.770 | 1.00 | 94.07 | | O |
| ANISOU | 1052 | O | LEU | A | 154 | 11818 | 7192 | 16732 | −2235 | 7339 | −3011 | O |
| ATOM | 1053 | CB | LEU | A | 154 | 107.424 | −47.315 | 93.855 | 1.00 | 80.66 | | C |
| ANISOU | 1053 | CB | LEU | A | 154 | 10058 | 6397 | 14191 | −1322 | 5870 | −2032 | C |
| ATOM | 1054 | CG | LEU | A | 154 | 106.643 | −46.621 | 92.724 | 1.00 | 83.42 | | C |
| ANISOU | 1054 | CG | LEU | A | 154 | 10831 | 6239 | 14627 | −861 | 6391 | −1465 | C |
| ATOM | 1055 | CD1 | LEU | A | 154 | 106.665 | −47.427 | 91.446 | 1.00 | 83.16 | | C |
| ANISOU | 1055 | CD1 | LEU | A | 154 | 10799 | 6384 | 14414 | −485 | 6118 | −990 | C |
| ATOM | 1056 | CD2 | LEU | A | 154 | 105.219 | −46.391 | 93.106 | 1.00 | 81.81 | | C |
| ANISOU | 1056 | CD2 | LEU | A | 154 | 10823 | 6008 | 14254 | −514 | 6379 | −1155 | C |
| ATOM | 1057 | N | GLU | A | 155 | 110.638 | −46.409 | 95.185 | 1.00 | 96.03 | | N |
| ANISOU | 1057 | N | GLU | A | 155 | 11273 | 8565 | 16649 | −2749 | 6356 | −3778 | N |
| ATOM | 1058 | CA | GLU | A | 155 | 111.431 | −45.308 | 95.762 | 1.00 | 99.18 | | C |
| ANISOU | 1058 | CA | GLU | A | 155 | 11548 | 8753 | 17384 | −3357 | 6983 | −4540 | C |
| ATOM | 1059 | C | GLU | A | 155 | 112.399 | −44.650 | 94.712 | 1.00 | 109.96 | | C |
| ANISOU | 1059 | C | GLU | A | 155 | 13017 | 9609 | 19154 | −3584 | 7665 | −4673 | C |
| ATOM | 1060 | O | GLU | A | 155 | 112.872 | −43.519 | 94.896 | 1.00 | 109.92 | | O |
| ANISOU | 1060 | O | GLU | A | 155 | 13066 | 9160 | 19540 | −4053 | 8445 | −5210 | O |
| ATOM | 1061 | CB | GLU | A | 155 | 112.189 | −45.793 | 97.020 | 1.00 | 100.88 | | C |
| ANISOU | 1061 | CB | GLU | A | 155 | 11183 | 9785 | 17361 | −3757 | 6491 | −5229 | C |
| ATOM | 1062 | CG | GLU | A | 155 | 112.540 | −44.693 | 98.027 | 1.00 | 119.83 | | C |
| ANISOU | 1062 | CG | GLU | A | 155 | 13418 | 12130 | 19982 | −4366 | 7026 | −6074 | C |
| ATOM | 1063 | CD | GLU | A | 155 | 113.795 | −43.843 | 97.830 | 1.00 | 163.54 | | C |
| ANISOU | 1063 | CD | GLU | A | 155 | 18754 | 17472 | 25912 | −4995 | 7702 | −6816 | C |
| ATOM | 1064 | OE1 | GLU | A | 155 | 114.721 | −44.275 | 97.101 | 1.00 | 172.06 | | O |
| ANISOU | 1064 | OE1 | GLU | A | 155 | 19648 | 18704 | 27021 | −5031 | 7600 | −6815 | O |
| ATOM | 1065 | OE2 | GLU | A | 155 | 113.856 | −42.740 | 98.426 | 1.00 | 160.35 | | O |
| ANISOU | 1065 | OE2 | GLU | A | 155 | 18366 | 16761 | 25798 | −5483 | 8368 | −7442 | O |
| ATOM | 1066 | N | HIS | A | 156 | 112.642 | −45.351 | 93.595 | 1.00 | 111.43 | | N |
| ANISOU | 1066 | N | HIS | A | 156 | 13255 | 9825 | 19258 | −3252 | 7422 | −4182 | N |
| ATOM | 1067 | CA | HIS | A | 156 | 113.532 | −44.909 | 92.509 | 1.00 | 113.70 | | C |
| ANISOU | 1067 | CA | HIS | A | 156 | 13643 | 9684 | 19873 | −3383 | 7983 | −4206 | C |
| ATOM | 1068 | C | HIS | A | 156 | 112.739 | −44.328 | 91.326 | 1.00 | 121.03 | | C |
| ANISOU | 1068 | C | HIS | A | 156 | 15154 | 9878 | 20953 | −2882 | 8548 | −3485 | C |
| ATOM | 1069 | O | HIS | A | 156 | 113.164 | −44.419 | 90.169 | 1.00 | 121.35 | | O |
| ANISOU | 1069 | O | HIS | A | 156 | 15322 | 9705 | 21081 | −2698 | 8740 | −3170 | O |
| ATOM | 1070 | CB | HIS | A | 156 | 114.439 | −46.083 | 92.068 | 1.00 | 114.75 | | C |
| ANISOU | 1070 | CB | HIS | A | 156 | 13407 | 10399 | 19794 | −3348 | 7351 | −4179 | C |
| ATOM | 1071 | CG | HIS | A | 156 | 114.917 | −46.918 | 93.218 | 1.00 | 118.16 | | C |
| ANISOU | 1071 | CG | HIS | A | 156 | 13294 | 11691 | 19910 | −3569 | 6638 | −4638 | C |
| ATOM | 1072 | ND1 | HIS | A | 156 | 115.909 | −46.466 | 94.077 | 1.00 | 119.92 | | N |
| ANISOU | 1072 | ND1 | HIS | A | 156 | 13088 | 12233 | 20242 | −4173 | 6816 | −5500 | N |
| ATOM | 1073 | CD2 | HIS | A | 156 | 114.469 | −48.117 | 93.659 | 1.00 | 119.81 | | C |
| ANISOU | 1073 | CD2 | HIS | A | 156 | 13338 | 12498 | 19687 | −3233 | 5821 | −4344 | C |
| ATOM | 1074 | CE1 | HIS | A | 156 | 116.052 | −47.415 | 94.987 | 1.00 | 119.30 | | C |
| ANISOU | 1074 | CE1 | HIS | A | 156 | 12588 | 12992 | 19750 | −4119 | 6067 | −5651 | C |
| ATOM | 1075 | NE2 | HIS | A | 156 | 115.201 | −48.424 | 94.782 | 1.00 | 119.62 | | N |
| ANISOU | 1075 | NE2 | HIS | A | 156 | 12802 | 13174 | 19477 | −3556 | 5482 | −4951 | N |
| ATOM | 1076 | N | MET | A | 157 | 111.589 | −43.722 | 91.634 | 1.00 | 119.18 | | N |
| ANISOU | 1076 | N | MET | A | 157 | 15257 | 9304 | 20720 | −2630 | 8825 | −3221 | N |
| ATOM | 1077 | CA | MET | A | 157 | 110.685 | −43.145 | 90.649 | 1.00 | 120.18 | | C |
| ANISOU | 1077 | CA | MET | A | 157 | 15915 | 8845 | 20902 | −2048 | 9348 | −2499 | C |
| ATOM | 1078 | C | MET | A | 157 | 110.329 | −41.712 | 91.005 | 1.00 | 129.12 | | C |
| ANISOU | 1078 | C | MET | A | 157 | 17419 | 9229 | 22410 | −2183 | 10334 | −2663 | C |
| ATOM | 1079 | O | MET | A | 157 | 110.060 | −41.437 | 92.186 | 1.00 | 129.59 | | O |
| ANISOU | 1079 | O | MET | A | 157 | 17363 | 9383 | 22491 | −2502 | 10296 | −3112 | O |
| ATOM | 1080 | CB | MET | A | 157 | 109.387 | −43.955 | 90.599 | 1.00 | 122.22 | | C |
| ANISOU | 1080 | CB | MET | A | 157 | 16228 | 9511 | 20698 | −1440 | 8659 | −1876 | C |
| ATOM | 1081 | CG | MET | A | 157 | 109.496 | −45.200 | 89.803 | 1.00 | 125.54 | | C |
| ANISOU | 1081 | CG | MET | A | 157 | 16467 | 10438 | 20795 | −1133 | 7957 | −1502 | C |
| ATOM | 1082 | SD | MET | A | 157 | 108.873 | −44.863 | 88.153 | 1.00 | 129.43 | | S |
| ANISOU | 1082 | SD | MET | A | 157 | 17391 | 10565 | 21221 | −404 | 8408 | −663 | S |
| ATOM | 1083 | CE | MET | A | 157 | 107.113 | −44.990 | 88.442 | 1.00 | 126.05 | | C |
| ANISOU | 1083 | CE | MET | A | 157 | 17113 | 10367 | 20415 | 172 | 8112 | −163 | C |
| ATOM | 1084 | N | PRO | A | 158 | 110.223 | −40.796 | 90.003 | 1.00 | 127.83 | | N |
| ANISOU | 1084 | N | PRO | A | 158 | 17736 | 8314 | 22520 | −1876 | 11250 | −2249 | N |
| ATOM | 1085 | CA | PRO | A | 158 | 109.792 | −39.415 | 90.319 | 1.00 | 128.16 | | C |
| ANISOU | 1085 | CA | PRO | A | 158 | 18205 | 7553 | 22935 | −1927 | 12297 | −2328 | C |
| ATOM | 1086 | C | PRO | A | 158 | 108.453 | −39.399 | 91.077 | 1.00 | 131.44 | | C |
| ANISOU | 1086 | C | PRO | A | 158 | 18724 | 8126 | 23092 | −1592 | 12011 | −2078 | C |
| ATOM | 1087 | O | PRO | A | 158 | 107.593 | −40.220 | 90.755 | 1.00 | 129.86 | | O |
| ANISOU | 1087 | O | PRO | A | 158 | 18487 | 8417 | 22437 | −1013 | 11295 | −1483 | O |
| ATOM | 1088 | CB | PRO | A | 158 | 109.688 | −38.743 | 88.941 | 1.00 | 130.09 | | C |
| ANISOU | 1088 | CB | PRO | A | 158 | 18967 | 7114 | 23348 | −1372 | 13153 | −1645 | C |
| ATOM | 1089 | CG | PRO | A | 158 | 109.698 | −39.872 | 87.936 | 1.00 | 133.97 | | C |
| ANISOU | 1089 | CG | PRO | A | 158 | 19294 | 8188 | 23420 | −897 | 12371 | −1095 | C |
| ATOM | 1090 | CD | PRO | A | 158 | 110.489 | −40.965 | 88.558 | 1.00 | 129.20 | | C |
| ANISOU | 1090 | CD | PRO | A | 158 | 18099 | 8325 | 22665 | −1442 | 11425 | −1679 | C |
| ATOM | 1091 | N | PRO | A | 159 | 108.266 | −38.528 | 92.108 | 1.00 | 129.63 | | N |
| ANISOU | 1091 | N | PRO | A | 159 | 18586 | 7536 | 23133 | −1984 | 12537 | −2575 | N |
| ATOM | 1092 | CA | PRO | A | 159 | 106.999 | −38.539 | 92.886 | 1.00 | 129.17 | | C |
| ANISOU | 1092 | CA | PRO | A | 159 | 18603 | 7654 | 22820 | −1683 | 12238 | −2362 | C |
| ATOM | 1093 | C | PRO | A | 159 | 105.678 | −38.375 | 92.103 | 1.00 | 131.61 | | C |
| ANISOU | 1093 | C | PRO | A | 159 | 19338 | 7783 | 22883 | −743 | 12386 | −1383 | C |
| ATOM | 1094 | O | PRO | A | 159 | 104.624 | −38.670 | 92.672 | 1.00 | 131.57 | | O |
| ANISOU | 1094 | O | PRO | A | 159 | 19295 | 8122 | 22571 | −459 | 11914 | −1175 | O |
| ATOM | 1095 | CB | PRO | A | 159 | 107.188 | −37.400 | 93.895 | 1.00 | 131.14 | | C |
| ANISOU | 1095 | CB | PRO | A | 159 | 18960 | 7359 | 23508 | −2283 | 13060 | −3076 | C |
| ATOM | 1096 | CG | PRO | A | 159 | 108.670 | −37.264 | 94.032 | 1.00 | 136.39 | | C |
| ANISOU | 1096 | CG | PRO | A | 159 | 19317 | 7997 | 24507 | −3086 | 13314 | −3910 | C |
| ATOM | 1097 | CD | PRO | A | 159 | 109.208 | −37.524 | 92.647 | 1.00 | 132.04 | | C |
| ANISOU | 1097 | CD | PRO | A | 159 | 18889 | 7296 | 23984 | −2768 | 13426 | −3424 | C |
| ATOM | 1098 | N | GLU | A | 160 | 105.721 | −37.949 | 90.814 | 1.00 | 126.60 | | N |
| ANISOU | 1098 | N | GLU | A | 160 | 19069 | 6697 | 22336 | −233 | 13008 | −785 | N |
| ATOM | 1099 | CA | GLU | A | 160 | 104.542 | −37.810 | 89.942 | 1.00 | 125.26 | | C |
| ANISOU | 1099 | CA | GLU | A | 160 | 19245 | 6493 | 21854 | 744 | 13153 | 170 | C |
| ATOM | 1100 | C | GLU | A | 160 | 104.159 | −39.189 | 89.358 | 1.00 | 125.72 | | C |
| ANISOU | 1100 | C | GLU | A | 160 | 18962 | 7470 | 21334 | 1160 | 12020 | 591 | C |
| ATOM | 1101 | O | GLU | A | 160 | 103.001 | −39.619 | 89.513 | 1.00 | 126.26 | | O |
| ANISOU | 1101 | O | GLU | A | 160 | 18968 | 8012 | 20991 | 1630 | 11500 | 967 | O |
| ATOM | 1102 | CB | GLU | A | 160 | 104.799 | −36.791 | 88.816 | 1.00 | 126.56 | | C |
| ANISOU | 1102 | CB | GLU | A | 160 | 19927 | 5849 | 22312 | 1156 | 14326 | 638 | C |
| ATOM | 1103 | N | LYS | A | 161 | 105.155 | −39.886 | 88.726 | 1.00 | 117.19 | | N |
| ANISOU | 1103 | N | LYS | A | 161 | 17643 | 6641 | 20244 | 936 | 11668 | 465 | N |
| ATOM | 1104 | CA | LYS | A | 161 | 105.056 | −41.225 | 88.125 | 1.00 | 114.99 | | C |
| ANISOU | 1104 | CA | LYS | A | 161 | 17029 | 7162 | 19501 | 1192 | 10683 | 738 | C |
| ATOM | 1105 | C | LYS | A | 161 | 104.602 | −42.257 | 89.174 | 1.00 | 114.80 | | C |
| ANISOU | 1105 | C | LYS | A | 161 | 16600 | 7839 | 19181 | 944 | 9671 | 435 | C |
| ATOM | 1106 | O | LYS | A | 161 | 103.888 | −43.212 | 88.850 | 1.00 | 114.94 | | O |
| ANISOU | 1106 | O | LYS | A | 161 | 16437 | 8478 | 18756 | 1330 | 8960 | 784 | O |
| ATOM | 1107 | CB | LYS | A | 161 | 106.412 | −41.631 | 87.523 | 1.00 | 117.04 | | C |
| ANISOU | 1107 | CB | LYS | A | 161 | 17113 | 7428 | 19931 | 816 | 10623 | 475 | C |
| ATOM | 1108 | N | GLN | A | 162 | 105.002 | −42.026 | 90.440 | 1.00 | 107.04 | | N |
| ANISOU | 1108 | N | GLN | A | 162 | 15478 | 6754 | 18440 | 300 | 9674 | −235 | N |
| ATOM | 1109 | CA | GLN | A | 162 | 104.656 | −42.819 | 91.612 | 1.00 | 104.80 | | C |
| ANISOU | 1109 | CA | GLN | A | 162 | 14853 | 7040 | 17927 | 29 | 8873 | −573 | C |
| ATOM | 1110 | C | GLN | A | 162 | 103.142 | −42.817 | 91.823 | 1.00 | 108.35 | | C |
| ANISOU | 1110 | C | GLN | A | 162 | 15438 | 7657 | 18072 | 563 | 8708 | −120 | C |
| ATOM | 1111 | O | GLN | A | 162 | 102.600 | −43.848 | 92.198 | 1.00 | 108.31 | | O |
| ANISOU | 1111 | O | GLN | A | 162 | 15167 | 8268 | 17717 | 633 | 7904 | −78 | O |
| ATOM | 1112 | CB | GLN | A | 162 | 105.339 | −42.218 | 92.846 | 1.00 | 105.16 | | C |
| ANISOU | 1112 | CB | GLN | A | 162 | 14789 | 6859 | 18308 | −692 | 9163 | −1359 | C |
| ATOM | 1113 | CG | GLN | A | 162 | 106.663 | −42.863 | 93.235 | 1.00 | 99.06 | | C |
| ANISOU | 1113 | CG | GLN | A | 162 | 13581 | 6464 | 17594 | −1307 | 8744 | −1986 | C |
| ATOM | 1114 | CD | GLN | A | 162 | 107.355 | −42.171 | 94.396 | 1.00 | 104.44 | | C |
| ANISOU | 1114 | CD | GLN | A | 162 | 14101 | 7008 | 18572 | −2017 | 9098 | −2826 | C |
| ATOM | 1115 | OE1 | GLN | A | 162 | 106.803 | −41.282 | 95.070 | 1.00 | 100.45 | | O |
| ANISOU | 1115 | OE1 | GLN | A | 162 | 13799 | 6134 | 18232 | −2111 | 9595 | −2996 | O |
| ATOM | 1116 | NE2 | GLN | A | 162 | 108.604 | −42.552 | 94.645 | 1.00 | 85.02 | | N |
| ANISOU | 1116 | NE2 | GLN | A | 162 | 11245 | 4883 | 16176 | −2537 | 8874 | −3406 | N |
| ATOM | 1117 | N | LYS | A | 163 | 102.465 | −41.655 | 91.594 | 1.00 | 104.84 | | N |
| ANISOU | 1117 | N | LYS | A | 163 | 15411 | 6656 | 17769 | 947 | 9517 | 215 | N |
| ATOM | 1118 | CA | LYS | A | 163 | 101.011 | −41.475 | 91.756 | 1.00 | 104.23 | | C |
| ANISOU | 1118 | CA | LYS | A | 163 | 15480 | 6708 | 17414 | 1511 | 9486 | 666 | C |
| ATOM | 1119 | C | LYS | A | 163 | 100.210 | −42.052 | 90.585 | 1.00 | 107.02 | | C |
| ANISOU | 1119 | C | LYS | A | 163 | 15814 | 7536 | 17313 | 2269 | 9163 | 1373 | C |
| ATOM | 1120 | O | LYS | A | 163 | 99.099 | −42.533 | 90.790 | 1.00 | 106.17 | | O |
| ANISOU | 1120 | O | LYS | A | 163 | 15580 | 7924 | 16836 | 2606 | 8688 | 1605 | O |
| ATOM | 1121 | CB | LYS | A | 163 | 100.648 | −40.001 | 92.013 | 1.00 | 106.22 | | C |
| ANISOU | 1121 | CB | LYS | A | 163 | 16182 | 6183 | 17994 | 1645 | 10529 | 738 | C |
| ATOM | 1122 | N | ARG | A | 164 | 100.781 | −42.031 | 89.371 | 1.00 | 103.23 | | N |
| ANISOU | 1122 | N | ARG | A | 164 | 15421 | 6959 | 16843 | 2514 | 9413 | 1669 | N |
| ATOM | 1123 | CA | ARG | A | 164 | 100.168 | −42.593 | 88.160 | 1.00 | 102.79 | | C |
| ANISOU | 1123 | CA | ARG | A | 164 | 15295 | 7434 | 16328 | 3206 | 9120 | 2276 | C |
| ATOM | 1124 | C | ARG | A | 164 | 100.036 | −44.120 | 88.292 | 1.00 | 103.18 | | C |
| ANISOU | 1124 | C | ARG | A | 164 | 14877 | 8298 | 16029 | 3010 | 8044 | 2091 | C |
| ATOM | 1125 | O | ARG | A | 164 | 98.971 | −44.664 | 87.999 | 1.00 | 103.31 | | O |
| ANISOU | 1125 | O | ARG | A | 164 | 14739 | 8904 | 15608 | 3462 | 7628 | 2403 | O |
| ATOM | 1126 | CB | ARG | A | 164 | 101.005 | −42.206 | 86.913 | 1.00 | 106.23 | | C |
| ANISOU | 1126 | CB | ARG | A | 164 | 15928 | 7536 | 16897 | 3417 | 9672 | 2550 | C |
| ATOM | 1127 | CG | ARG | A | 164 | 100.632 | −42.906 | 85.595 | 1.00 | 122.79 | | C |
| ANISOU | 1127 | CG | ARG | A | 164 | 17878 | 10270 | 18507 | 4039 | 9313 | 3073 | C |
| ATOM | 1128 | CD | ARG | A | 164 | 101.391 | −42.334 | 84.405 | 1.00 | 138.94 | | C |
| ANISOU | 1128 | CD | ARG | A | 164 | 20188 | 11914 | 20689 | 4322 | 9995 | 3401 | C |
| ATOM | 1129 | NE | ARG | A | 164 | 100.940 | −40.979 | 84.069 | 1.00 | 155.55 | | N |
| ANISOU | 1129 | NE | ARG | A | 164 | 22775 | 13431 | 22894 | 4925 | 11029 | 3906 | N |
| ATOM | 1130 | CZ | ARG | A | 164 | 101.327 | −40.297 | 82.996 | 1.00 | 175.43 | | C |
| ANISOU | 1130 | CZ | ARG | A | 164 | 25337 | 16081 | 25237 | 5000 | 10865 | 3982 | C |
| ATOM | 1131 | NH1 | ARG | A | 164 | 102.178 | −40.838 | 82.129 | 1.00 | 166.44 | | N |
| ANISOU | 1131 | NH1 | ARG | A | 164 | 24349 | 14584 | 24305 | 5292 | 11617 | 4355 | N |
| ATOM | 1132 | NH2 | ARG | A | 164 | 100.863 | −39.075 | 82.773 | 1.00 | 161.63 | | N |
| ANISOU | 1132 | NH2 | ARG | A | 164 | 24092 | 13691 | 23628 | 5643 | 12000 | 4525 | N |
| ATOM | 1133 | N | ILE | A | 165 | 101.116 | −44.788 | 88.756 | 1.00 | 96.38 | | N |
| ANISOU | 1133 | N | ILE | A | 165 | 13785 | 7469 | 15364 | 2342 | 7650 | 1565 | N |
| ATOM | 1134 | CA | ILE | A | 165 | 101.197 | −46.236 | 88.961 | 1.00 | 94.68 | | C |
| ANISOU | 1134 | CA | ILE | A | 165 | 13173 | 7900 | 14901 | 2099 | 6742 | 1353 | C |
| ATOM | 1135 | C | ILE | A | 165 | 100.256 | −46.703 | 90.066 | 1.00 | 96.90 | | C |
| ANISOU | 1135 | C | ILE | A | 165 | 13299 | 8524 | 14996 | 2016 | 6262 | 1202 | C |
| ATOM | 1136 | O | ILE | A | 165 | 99.492 | −47.637 | 89.835 | 1.00 | 96.70 | | O |
| ANISOU | 1136 | O | ILE | A | 165 | 13065 | 9067 | 14609 | 2242 | 5725 | 1364 | O |
| ATOM | 1137 | CB | ILE | A | 165 | 102.676 | −46.683 | 89.187 | 1.00 | 97.48 | | C |
| ANISOU | 1137 | CB | ILE | A | 165 | 13353 | 8160 | 15525 | 1479 | 6571 | 869 | C |
| ATOM | 1138 | CG1 | ILE | A | 165 | 103.565 | −46.446 | 87.929 | 1.00 | 97.81 | | C |
| ANISOU | 1138 | CG1 | ILE | A | 165 | 13503 | 7964 | 15696 | 1593 | 6954 | 1053 | C |
| ATOM | 1139 | CG2 | ILE | A | 165 | 102.796 | −48.128 | 89.700 | 1.00 | 97.82 | | C |
| ANISOU | 1139 | CG2 | ILE | A | 165 | 13023 | 8786 | 15359 | 1201 | 5713 | 614 | C |
| ATOM | 1140 | CD1 | ILE | A | 165 | 102.989 | −46.922 | 86.540 | 1.00 | 104.11 | | C |
| ANISOU | 1140 | CD1 | ILE | A | 165 | 14290 | 9157 | 16112 | 2219 | 6808 | 1613 | C |
| ATOM | 1141 | N | ALA | A | 166 | 100.302 | −46.043 | 91.254 | 1.00 | 91.94 | | N |
| ANISOU | 1141 | N | ALA | A | 166 | 12765 | 7554 | 14615 | 1675 | 6496 | 862 | N |
| ATOM | 1142 | CA | ALA | A | 166 | 99.496 | −46.357 | 92.438 | 1.00 | 90.55 | | C |
| ANISOU | 1142 | CA | ALA | A | 166 | 12471 | 7632 | 14303 | 1553 | 6117 | 680 | C |
| ATOM | 1143 | C | ALA | A | 166 | 98.002 | −46.361 | 92.130 | 1.00 | 94.81 | | C |
| ANISOU | 1143 | C | ALA | A | 166 | 13055 | 8471 | 14499 | 2143 | 6051 | 1136 | C |
| ATOM | 1144 | O | ALA | A | 166 | 97.314 | −47.324 | 92.495 | 1.00 | 94.61 | | O |
| ANISOU | 1144 | O | ALA | A | 166 | 12793 | 8962 | 14193 | 2149 | 5455 | 1106 | O |
| ATOM | 1145 | CB | ALA | A | 166 | 99.823 | −45.396 | 93.577 | 1.00 | 90.82 | | C |
| ANISOU | 1145 | CB | ALA | A | 166 | 12641 | 7195 | 14670 | 1147 | 6548 | 263 | C |
| ATOM | 1146 | N | GLN | A | 167 | 97.520 | −45.325 | 91.396 | 1.00 | 91.21 | | N |
| ANISOU | 1146 | N | GLN | A | 167 | 12887 | 7725 | 14045 | 2669 | 6689 | 1569 | N |
| ATOM | 1147 | CA | GLN | A | 167 | 96.121 | −45.188 | 90.983 | 1.00 | 90.97 | | C |
| ANISOU | 1147 | CA | GLN | A | 167 | 12877 | 8044 | 13644 | 3334 | 6707 | 2043 | C |
| ATOM | 1148 | C | GLN | A | 167 | 95.725 | −46.346 | 90.084 | 1.00 | 93.58 | | C |
| ANISOU | 1148 | C | GLN | A | 167 | 12889 | 9120 | 13547 | 3596 | 6115 | 2234 | C |
| ATOM | 1149 | O | GLN | A | 167 | 94.783 | −47.075 | 90.392 | 1.00 | 93.13 | | O |
| ANISOU | 1149 | O | GLN | A | 167 | 12590 | 9609 | 13187 | 3668 | 5625 | 2208 | O |
| ATOM | 1150 | CB | GLN | A | 167 | 95.883 | −43.850 | 90.261 | 1.00 | 92.69 | | C |
| ANISOU | 1150 | CB | GLN | A | 167 | 13478 | 7802 | 13937 | 3917 | 7586 | 2521 | C |
| ATOM | 1151 | CG | GLN | A | 167 | 94.428 | −43.402 | 90.269 | 1.00 | 113.87 | | C |
| ANISOU | 1151 | CG | GLN | A | 167 | 16223 | 10742 | 16301 | 4571 | 7739 | 2939 | C |
| ATOM | 1152 | CD | GLN | A | 167 | 94.034 | −42.996 | 91.658 | 1.00 | 142.95 | | C |
| ANISOU | 1152 | CD | GLN | A | 167 | 19992 | 14137 | 20184 | 4247 | 7810 | 2627 | C |
| ATOM | 1153 | OE1 | GLN | A | 167 | 93.474 | −43.786 | 92.426 | 1.00 | 141.34 | | O |
| ANISOU | 1153 | OE1 | GLN | A | 167 | 19518 | 14381 | 19803 | 4017 | 7182 | 2384 | O |
| ATOM | 1154 | NE2 | GLN | A | 167 | 94.360 | −41.766 | 92.019 | 1.00 | 140.13 | | N |
| ANISOU | 1154 | NE2 | GLN | A | 167 | 20022 | 13001 | 20222 | 4188 | 8618 | 2596 | N |
| ATOM | 1155 | N | GLU | A | 168 | 96.483 | −46.539 | 88.997 | 1.00 | 89.16 | | N |
| ANISOU | 1155 | N | GLU | A | 168 | 12318 | 8576 | 12983 | 3684 | 6180 | 2368 | N |
| ATOM | 1156 | CA | GLU | A | 168 | 96.277 | −47.627 | 88.051 | 1.00 | 87.85 | | C |
| ANISOU | 1156 | CA | GLU | A | 168 | 11846 | 9092 | 12443 | 3873 | 5677 | 2483 | C |
| ATOM | 1157 | C | GLU | A | 168 | 96.168 | −48.961 | 88.793 | 1.00 | 89.67 | | C |
| ANISOU | 1157 | C | GLU | A | 168 | 11752 | 9727 | 12593 | 3394 | 4939 | 2075 | C |
| ATOM | 1158 | O | GLU | A | 168 | 95.234 | −49.705 | 88.534 | 1.00 | 89.39 | | O |
| ANISOU | 1158 | O | GLU | A | 168 | 11460 | 10314 | 12191 | 3596 | 4561 | 2132 | O |
| ATOM | 1159 | CB | GLU | A | 168 | 97.406 | −47.648 | 87.003 | 1.00 | 88.78 | | C |
| ANISOU | 1159 | CB | GLU | A | 168 | 12021 | 9033 | 12677 | 3860 | 5863 | 2566 | C |
| ATOM | 1160 | CG | GLU | A | 168 | 97.412 | −48.895 | 86.143 | 1.00 | 97.88 | | C |
| ANISOU | 1160 | CG | GLU | A | 168 | 12838 | 10843 | 13508 | 3895 | 5313 | 2554 | C |
| ATOM | 1161 | CD | GLU | A | 168 | 98.040 | −48.777 | 84.767 | 1.00 | 120.21 | | C |
| ANISOU | 1161 | CD | GLU | A | 168 | 15715 | 13700 | 16261 | 4196 | 5560 | 2831 | C |
| ATOM | 1162 | OE1 | GLU | A | 168 | 99.227 | −48.382 | 84.668 | 1.00 | 112.05 | | O |
| ANISOU | 1162 | OE1 | GLU | A | 168 | 14872 | 12106 | 15596 | 3927 | 5880 | 2743 | O |
| ATOM | 1163 | OE2 | GLU | A | 168 | 97.339 | −49.105 | 83.783 | 1.00 | 112.48 | | O |
| ANISOU | 1163 | OE2 | GLU | A | 168 | 14549 | 13361 | 14830 | 4694 | 5429 | 3105 | O |
| ATOM | 1164 | N | THR | A | 169 | 97.082 | −49.228 | 89.745 | 1.00 | 84.18 | | N |
| ANISOU | 1164 | N | THR | A | 169 | 11061 | 8701 | 12223 | 2784 | 4787 | 1659 | N |
| ATOM | 1165 | CA | THR | A | 169 | 97.109 | −50.467 | 90.510 | 1.00 | 83.19 | | C |
| ANISOU | 1165 | CA | THR | A | 169 | 10681 | 8884 | 12044 | 2369 | 4178 | 1316 | C |
| ATOM | 1166 | C | THR | A | 169 | 95.825 | −50.663 | 91.260 | 1.00 | 88.75 | | C |
| ANISOU | 1166 | C | THR | A | 169 | 11300 | 9883 | 12540 | 2470 | 3971 | 1308 | C |
| ATOM | 1167 | O | THR | A | 169 | 95.258 | −51.758 | 91.204 | 1.00 | 89.43 | | O |
| ANISOU | 1167 | O | THR | A | 169 | 11139 | 10453 | 12386 | 2442 | 3537 | 1238 | O |
| ATOM | 1168 | CB | THR | A | 169 | 98.302 | −50.503 | 91.443 | 1.00 | 86.61 | | C |
| ANISOU | 1168 | CB | THR | A | 169 | 11139 | 8956 | 12813 | 1813 | 4140 | 921 | C |
| ATOM | 1169 | OG1 | THR | A | 169 | 99.495 | −50.395 | 90.674 | 1.00 | 91.56 | | O |
| ANISOU | 1169 | OG1 | THR | A | 169 | 11803 | 9365 | 13621 | 1711 | 4319 | 906 | O |
| ATOM | 1170 | CG2 | THR | A | 169 | 98.361 | −51.779 | 92.253 | 1.00 | 81.41 | | C |
| ANISOU | 1170 | CG2 | THR | A | 169 | 10247 | 8613 | 12071 | 1478 | 3571 | 640 | C |
| ATOM | 1171 | N | LEU | A | 170 | 95.347 | −49.601 | 91.949 | 1.00 | 84.91 | | N |
| ANISOU | 1171 | N | LEU | A | 170 | 11018 | 9087 | 12156 | 2576 | 4328 | 1361 | N |
| ATOM | 1172 | CA | LEU | A | 170 | 94.106 | −49.637 | 92.746 | 1.00 | 83.17 | | C |
| ANISOU | 1172 | CA | LEU | A | 170 | 10740 | 9102 | 11759 | 2681 | 4187 | 1356 | C |
| ATOM | 1173 | C | LEU | A | 170 | 92.852 | −49.818 | 91.914 | 1.00 | 88.01 | | C |
| ANISOU | 1173 | C | LEU | A | 170 | 11193 | 10287 | 11961 | 3216 | 4114 | 1669 | C |
| ATOM | 1174 | O | LEU | A | 170 | 91.916 | −50.491 | 92.340 | 1.00 | 87.07 | | O |
| ANISOU | 1174 | O | LEU | A | 170 | 10866 | 10594 | 11624 | 3196 | 3778 | 1568 | O |
| ATOM | 1175 | CB | LEU | A | 170 | 93.984 | −48.406 | 93.652 | 1.00 | 82.09 | | C |
| ANISOU | 1175 | CB | LEU | A | 170 | 10876 | 8461 | 11855 | 2657 | 4641 | 1319 | C |
| ATOM | 1176 | CG | LEU | A | 170 | 94.938 | −48.396 | 94.826 | 1.00 | 85.40 | | C |
| ANISOU | 1176 | CG | LEU | A | 170 | 11331 | 8533 | 12584 | 2061 | 4590 | 869 | C |
| ATOM | 1177 | CD1 | LEU | A | 170 | 94.965 | −47.065 | 95.493 | 1.00 | 85.52 | | C |
| ANISOU | 1177 | CD1 | LEU | A | 170 | 11620 | 8011 | 12863 | 2013 | 5158 | 788 | C |
| ATOM | 1178 | CD2 | LEU | A | 170 | 94.610 | −49.477 | 95.818 | 1.00 | 86.06 | | C |
| ANISOU | 1178 | CD2 | LEU | A | 170 | 11194 | 8974 | 12531 | 1774 | 4029 | 611 | C |
| ATOM | 1179 | N | GLU | A | 171 | 92.853 | −49.242 | 90.716 | 1.00 | 86.65 | | N |
| ANISOU | 1179 | N | GLU | A | 171 | 11091 | 10170 | 11662 | 3698 | 4443 | 2032 | N |
| ATOM | 1180 | CA | GLU | A | 171 | 91.735 | −49.304 | 89.790 | 1.00 | 86.98 | | C |
| ANISOU | 1180 | CA | GLU | A | 171 | 10936 | 10872 | 11242 | 4297 | 4415 | 2346 | C |
| ATOM | 1181 | C | GLU | A | 171 | 91.707 | −50.607 | 88.974 | 1.00 | 88.55 | | C |
| ANISOU | 1181 | C | GLU | A | 171 | 10762 | 11721 | 11162 | 4213 | 3935 | 2207 | C |
| ATOM | 1182 | O | GLU | A | 171 | 90.663 | −51.243 | 88.864 | 1.00 | 88.64 | | O |
| ANISOU | 1182 | O | GLU | A | 171 | 10459 | 12394 | 10827 | 4336 | 3643 | 2135 | O |
| ATOM | 1183 | CB | GLU | A | 171 | 91.717 | −48.042 | 88.883 | 1.00 | 88.89 | | C |
| ANISOU | 1183 | CB | GLU | A | 171 | 11432 | 10920 | 11421 | 4952 | 5051 | 2851 | C |
| ATOM | 1184 | CG | GLU | A | 171 | 91.588 | −46.745 | 89.680 | 1.00 | 103.54 | | C |
| ANISOU | 1184 | CG | GLU | A | 171 | 13667 | 12118 | 13554 | 5058 | 5622 | 2979 | C |
| ATOM | 1185 | CD | GLU | A | 171 | 90.878 | −45.570 | 89.040 | 1.00 | 135.31 | | C |
| ANISOU | 1185 | CD | GLU | A | 171 | 17901 | 16134 | 17378 | 5882 | 6247 | 3545 | C |
| ATOM | 1186 | OE1 | GLU | A | 171 | 89.761 | −45.244 | 89.507 | 1.00 | 143.49 | | O |
| ANISOU | 1186 | OE1 | GLU | A | 171 | 18902 | 17400 | 18217 | 6198 | 6294 | 3670 | O |
| ATOM | 1187 | OE2 | GLU | A | 171 | 91.436 | −44.963 | 88.095 | 1.00 | 125.98 | | O |
| ANISOU | 1187 | OE2 | GLU | A | 171 | 16930 | 14706 | 16231 | 6241 | 6725 | 3884 | O |
| ATOM | 1188 | N | ILE | A | 172 | 92.851 | −51.020 | 88.449 | 1.00 | 83.03 | | N |
| ANISOU | 1188 | N | ILE | A | 172 | 10085 | 10832 | 10631 | 3960 | 3876 | 2118 | N |
| ATOM | 1189 | CA | ILE | A | 172 | 92.964 | −52.165 | 87.555 | 1.00 | 82.49 | | C |
| ANISOU | 1189 | CA | ILE | A | 172 | 9707 | 11300 | 10336 | 3888 | 3519 | 1994 | C |
| ATOM | 1190 | C | ILE | A | 172 | 93.509 | −53.461 | 88.185 | 1.00 | 83.54 | | C |
| ANISOU | 1190 | C | ILE | A | 172 | 9708 | 11375 | 10657 | 3243 | 3087 | 1560 | C |
| ATOM | 1191 | O | ILE | A | 172 | 92.810 | −54.470 | 88.174 | 1.00 | 83.58 | | O |
| ANISOU | 1191 | O | ILE | A | 172 | 9427 | 11883 | 10448 | 3125 | 2768 | 1348 | O |
| ATOM | 1192 | CB | ILE | A | 172 | 93.815 | −51.745 | 86.311 | 1.00 | 86.41 | | C |
| ANISOU | 1192 | CB | ILE | A | 172 | 10311 | 11693 | 10830 | 4165 | 3802 | 2263 | C |
| ATOM | 1193 | CG1 | ILE | A | 172 | 93.284 | −50.443 | 85.634 | 1.00 | 87.53 | | C |
| ANISOU | 1193 | CG1 | ILE | A | 172 | 10625 | 11872 | 10760 | 4911 | 4328 | 2779 | C |
| ATOM | 1194 | CG2 | ILE | A | 172 | 93.992 | −52.878 | 85.292 | 1.00 | 87.74 | | C |
| ANISOU | 1194 | CG2 | ILE | A | 172 | 10162 | 12419 | 10757 | 4102 | 3471 | 2127 | C |
| ATOM | 1195 | CD1 | ILE | A | 172 | 91.677 | −50.348 | 85.335 | 1.00 | 98.31 | | C |
| ANISOU | 1195 | CD1 | ILE | A | 172 | 11713 | 14069 | 11570 | 5511 | 4265 | 2981 | C |
| ATOM | 1196 | N | TYR | A | 173 | 94.756 | −53.460 | 88.662 | 1.00 | 77.60 | | N |
| ANISOU | 1196 | N | TYR | A | 173 | 9145 | 10055 | 10284 | 2852 | 3117 | 1424 | N |
| ATOM | 1197 | CA | TYR | A | 173 | 95.425 | −54.680 | 89.091 | 1.00 | 76.70 | | C |
| ANISOU | 1197 | CA | TYR | A | 173 | 8919 | 9909 | 10314 | 2356 | 2766 | 1098 | C |
| ATOM | 1198 | C | TYR | A | 173 | 94.969 | −55.260 | 90.427 | 1.00 | 77.07 | | C |
| ANISOU | 1198 | C | TYR | A | 173 | 8927 | 9937 | 10417 | 2043 | 2530 | 850 | C |
| ATOM | 1199 | O | TYR | A | 173 | 94.679 | −56.449 | 90.441 | 1.00 | 75.69 | | O |
| ANISOU | 1199 | O | TYR | A | 173 | 8561 | 10057 | 10142 | 1857 | 2262 | 663 | O |
| ATOM | 1200 | CB | TYR | A | 173 | 96.953 | −54.515 | 89.045 | 1.00 | 78.49 | | C |
| ANISOU | 1200 | CB | TYR | A | 173 | 9300 | 9659 | 10863 | 2107 | 2872 | 1043 | C |
| ATOM | 1201 | CG | TYR | A | 173 | 97.418 | −54.112 | 87.667 | 1.00 | 80.48 | | C |
| ANISOU | 1201 | CG | TYR | A | 173 | 9583 | 9941 | 11054 | 2398 | 3103 | 1282 | C |
| ATOM | 1202 | CD2 | TYR | A | 173 | 97.799 | −52.806 | 87.397 | 1.00 | 81.42 | | C |
| ANISOU | 1202 | CD2 | TYR | A | 173 | 9957 | 9660 | 11320 | 2627 | 3574 | 1517 | C |
| ATOM | 1203 | CD1 | TYR | A | 173 | 97.359 | −55.004 | 86.601 | 1.00 | 82.89 | | C |
| ANISOU | 1203 | CD1 | TYR | A | 173 | 9668 | 10695 | 11131 | 2480 | 2910 | 1281 | C |
| ATOM | 1204 | CE2 | TYR | A | 173 | 98.158 | −52.406 | 86.112 | 1.00 | 82.55 | | C |
| ANISOU | 1204 | CE2 | TYR | A | 173 | 10154 | 9831 | 11381 | 2961 | 3844 | 1790 | C |
| ATOM | 1205 | CE1 | TYR | A | 173 | 97.708 | −54.613 | 85.307 | 1.00 | 84.87 | | C |
| ANISOU | 1205 | CE1 | TYR | A | 173 | 9937 | 11042 | 11270 | 2804 | 3128 | 1525 | C |
| ATOM | 1206 | CZ | TYR | A | 173 | 98.126 | −53.314 | 85.069 | 1.00 | 88.93 | | C |
| ANISOU | 1206 | CZ | TYR | A | 173 | 10726 | 11136 | 11928 | 3062 | 3595 | 1804 | C |
| ATOM | 1207 | OH | TYR | A | 173 | 98.473 | −52.886 | 83.807 | 1.00 | 85.75 | | O |
| ANISOU | 1207 | OH | TYR | A | 173 | 10376 | 10795 | 11409 | 3429 | 3873 | 2092 | O |
| ATOM | 1208 | N | ALA | A | 174 | 94.899 | −54.486 | 91.523 | 1.00 | 73.20 | | N |
| ANISOU | 1208 | N | ALA | A | 174 | 8617 | 9111 | 10086 | 1975 | 2662 | 831 | N |
| ATOM | 1209 | CA | ALA | A | 174 | 94.429 | −55.046 | 92.795 | 1.00 | 73.33 | | C |
| ANISOU | 1209 | CA | ALA | A | 174 | 8595 | 9151 | 10117 | 1718 | 2445 | 618 | C |
| ATOM | 1210 | C | ALA | A | 174 | 93.063 | −55.739 | 92.613 | 1.00 | 78.49 | | C |
| ANISOU | 1210 | C | ALA | A | 174 | 9026 | 10319 | 10476 | 1843 | 2280 | 592 | C |
| ATOM | 1211 | O | ALA | A | 174 | 92.965 | −56.914 | 92.978 | 1.00 | 78.44 | | O |
| ANISOU | 1211 | O | ALA | A | 174 | 8898 | 10448 | 10456 | 1576 | 2053 | 384 | O |
| ATOM | 1212 | CB | ALA | A | 174 | 94.363 | −53.978 | 93.871 | 1.00 | 74.00 | | C |
| ANISOU | 1212 | CB | ALA | A | 174 | 8880 | 8884 | 10355 | 1692 | 2650 | 605 | C |
| ATOM | 1213 | N | PRO | A | 175 | 92.056 | −55.131 | 91.923 | 1.00 | 75.57 | | N |
| ANISOU | 1213 | N | PRO | A | 175 | 8571 | 10291 | 9849 | 2257 | 2419 | 788 | N |
| ATOM | 1214 | CA | PRO | A | 175 | 90.792 | −55.853 | 91.696 | 1.00 | 75.18 | | C |
| ANISOU | 1214 | CA | PRO | A | 175 | 8227 | 10847 | 9493 | 2333 | 2253 | 676 | C |
| ATOM | 1215 | C | PRO | A | 175 | 90.937 | −57.130 | 90.861 | 1.00 | 75.70 | | C |
| ANISOU | 1215 | C | PRO | A | 175 | 8036 | 11276 | 9449 | 2149 | 2060 | 468 | C |
| ATOM | 1216 | O | PRO | A | 175 | 90.241 | −58.105 | 91.132 | 1.00 | 73.07 | | O |
| ANISOU | 1216 | O | PRO | A | 175 | 7504 | 11233 | 9026 | 1929 | 1916 | 204 | O |
| ATOM | 1217 | CB | PRO | A | 175 | 89.919 | −54.805 | 90.997 | 1.00 | 77.54 | | C |
| ANISOU | 1217 | CB | PRO | A | 175 | 8471 | 11484 | 9506 | 2901 | 2473 | 971 | C |
| ATOM | 1218 | CG | PRO | A | 175 | 90.503 | −53.494 | 91.416 | 1.00 | 82.36 | | C |
| ANISOU | 1218 | CG | PRO | A | 175 | 9443 | 11487 | 10361 | 3043 | 2801 | 1213 | C |
| ATOM | 1219 | CD | PRO | A | 175 | 91.962 | −53.757 | 91.386 | 1.00 | 77.61 | | C |
| ANISOU | 1219 | CD | PRO | A | 175 | 8988 | 10432 | 10068 | 2700 | 2777 | 1114 | C |
| ATOM | 1220 | N | LEU | A | 176 | 91.844 | −57.129 | 89.861 | 1.00 | 72.72 | | N |
| ANISOU | 1220 | N | LEU | A | 176 | 7676 | 10855 | 9102 | 2214 | 2103 | 563 | N |
| ATOM | 1221 | CA | LEU | A | 176 | 92.113 | −58.289 | 89.002 | 1.00 | 72.62 | | C |
| ANISOU | 1221 | CA | LEU | A | 176 | 7442 | 11141 | 9010 | 2032 | 1962 | 358 | C |
| ATOM | 1222 | C | LEU | A | 176 | 92.707 | −59.438 | 89.796 | 1.00 | 75.78 | | C |
| ANISOU | 1222 | C | LEU | A | 176 | 7906 | 11212 | 9674 | 1545 | 1827 | 101 | C |
| ATOM | 1223 | O | LEU | A | 176 | 92.412 | −60.596 | 89.505 | 1.00 | 74.43 | | O |
| ANISOU | 1223 | O | LEU | A | 176 | 7537 | 11305 | 9438 | 1322 | 1751 | −164 | O |
| ATOM | 1224 | CB | LEU | A | 176 | 93.062 | −57.914 | 87.861 | 1.00 | 73.02 | | C |
| ANISOU | 1224 | CB | LEU | A | 176 | 7546 | 11134 | 9065 | 2223 | 2067 | 553 | C |
| ATOM | 1225 | CG | LEU | A | 176 | 92.403 | −57.364 | 86.628 | 1.00 | 78.83 | | C |
| ANISOU | 1225 | CG | LEU | A | 176 | 8085 | 12459 | 9408 | 2726 | 2175 | 748 | C |
| ATOM | 1226 | CD1 | LEU | A | 176 | 93.398 | −56.654 | 85.775 | 1.00 | 78.70 | | C |
| ANISOU | 1226 | CD1 | LEU | A | 176 | 8246 | 12198 | 9457 | 2973 | 2377 | 1042 | C |
| ATOM | 1227 | CD2 | LEU | A | 176 | 91.720 | −58.470 | 85.843 | 1.00 | 84.95 | | C |
| ANISOU | 1227 | CD2 | LEU | A | 176 | 8443 | 13965 | 9869 | 2640 | 2001 | 434 | C |
| ATOM | 1228 | N | ALA | A | 177 | 93.548 | −59.110 | 90.802 | 1.00 | 73.19 | | N |
| ANISOU | 1228 | N | ALA | A | 177 | 7848 | 10331 | 9630 | 1397 | 1839 | 169 | N |
| ATOM | 1229 | CA | ALA | A | 177 | 94.203 | −60.076 | 91.684 | 1.00 | 73.14 | | C |
| ANISOU | 1229 | CA | ALA | A | 177 | 7927 | 10024 | 9837 | 1044 | 1738 | 6 | C |
| ATOM | 1230 | C | ALA | A | 177 | 93.147 | −60.726 | 92.498 | 1.00 | 78.22 | | C |
| ANISOU | 1230 | C | ALA | A | 177 | 8499 | 10811 | 10411 | 907 | 1697 | −163 | C |
| ATOM | 1231 | O | ALA | A | 177 | 93.156 | −61.944 | 92.639 | 1.00 | 75.42 | | O |
| ANISOU | 1231 | O | ALA | A | 177 | 8091 | 10457 | 10106 | 666 | 1680 | −347 | O |
| ATOM | 1232 | CB | ALA | A | 177 | 95.195 | −59.373 | 92.596 | 1.00 | 73.97 | | C |
| ANISOU | 1232 | CB | ALA | A | 177 | 8264 | 9674 | 10168 | 988 | 1762 | 97 | C |
| ATOM | 1233 | N | HIS | A | 178 | 92.195 | −59.891 | 92.995 | 1.00 | 79.74 | | N |
| ANISOU | 1233 | N | HIS | A | 178 | 8696 | 11110 | 10491 | 1077 | 1735 | −95 | N |
| ATOM | 1234 | CA | HIS | A | 178 | 91.022 | −60.216 | 93.821 | 1.00 | 80.98 | | C |
| ANISOU | 1234 | CA | HIS | A | 178 | 8783 | 11426 | 10559 | 997 | 1724 | −233 | C |
| ATOM | 1235 | C | HIS | A | 178 | 90.136 | −61.268 | 93.131 | 1.00 | 84.22 | | C |
| ANISOU | 1235 | C | HIS | A | 178 | 8897 | 12305 | 10797 | 875 | 1727 | −498 | C |
| ATOM | 1236 | O | HIS | A | 178 | 89.852 | −62.318 | 93.709 | 1.00 | 82.61 | | O |
| ANISOU | 1236 | O | HIS | A | 178 | 8679 | 12037 | 10673 | 589 | 1761 | −713 | O |
| ATOM | 1237 | CB | HIS | A | 178 | 90.294 | −58.882 | 94.126 | 1.00 | 82.74 | | C |
| ANISOU | 1237 | CB | HIS | A | 178 | 9053 | 11711 | 10672 | 1295 | 1799 | −58 | C |
| ATOM | 1238 | CG | HIS | A | 178 | 88.870 | −58.960 | 94.583 | 1.00 | 87.24 | | C |
| ANISOU | 1238 | CG | HIS | A | 178 | 9472 | 12612 | 11063 | 1333 | 1803 | −168 | C |
| ATOM | 1239 | ND1 | HIS | A | 178 | 87.848 | −58.378 | 93.847 | 1.00 | 89.37 | | N |
| ANISOU | 1239 | ND1 | HIS | A | 178 | 9523 | 13389 | 11043 | 1667 | 1856 | −103 | N |
| ATOM | 1240 | CD2 | HIS | A | 178 | 88.352 | −59.455 | 95.734 | 1.00 | 89.48 | | C |
| ANISOU | 1240 | CD2 | HIS | A | 178 | 9799 | 12795 | 11406 | 1119 | 1784 | −311 | C |
| ATOM | 1241 | CE1 | HIS | A | 178 | 86.746 | −58.577 | 94.549 | 1.00 | 88.92 | | C |
| ANISOU | 1241 | CE1 | HIS | A | 178 | 9357 | 13538 | 10891 | 1608 | 1848 | −247 | C |
| ATOM | 1242 | NE2 | HIS | A | 178 | 86.999 | −59.228 | 95.687 | 1.00 | 89.23 | | N |
| ANISOU | 1242 | NE2 | HIS | A | 178 | 9562 | 13195 | 11148 | 1265 | 1816 | −377 | N |
| ATOM | 1243 | N | ARG | A | 179 | 89.811 | −61.006 | 91.853 | 1.00 | 81.78 | | N |
| ANISOU | 1243 | N | ARG | A | 179 | 8353 | 12464 | 10256 | 1090 | 1731 | −495 | N |
| ATOM | 1244 | CA | ARG | A | 179 | 89.015 | −61.821 | 90.935 | 1.00 | 81.79 | | C |
| ANISOU | 1244 | CA | ARG | A | 179 | 7983 | 13070 | 10023 | 1004 | 1739 | −804 | C |
| ATOM | 1245 | C | ARG | A | 179 | 89.734 | −63.101 | 90.476 | 1.00 | 84.15 | | C |
| ANISOU | 1245 | C | ARG | A | 179 | 8254 | 13242 | 10477 | 656 | 1773 | −1043 | C |
| ATOM | 1246 | O | ARG | A | 179 | 89.099 | −63.993 | 89.917 | 1.00 | 83.07 | | O |
| ANISOU | 1246 | O | ARG | A | 179 | 7824 | 13523 | 10217 | 447 | 1838 | −1411 | O |
| ATOM | 1247 | CB | ARG | A | 179 | 88.597 | −60.959 | 89.731 | 1.00 | 83.94 | | C |
| ANISOU | 1247 | CB | ARG | A | 179 | 8021 | 13930 | 9944 | 1449 | 1732 | −658 | C |
| ATOM | 1248 | CG | ARG | A | 179 | 87.417 | −60.017 | 90.009 | 1.00 | 100.26 | | C |
| ANISOU | 1248 | CG | ARG | A | 179 | 9974 | 16370 | 11750 | 1806 | 1757 | −530 | C |
| ATOM | 1249 | N | LEU | A | 180 | 91.050 | −63.180 | 90.677 | 1.00 | 81.10 | | N |
| ANISOU | 1249 | N | LEU | A | 180 | 8147 | 12314 | 10353 | 592 | 1759 | −864 | N |
| ATOM | 1250 | CA | LEU | A | 180 | 91.822 | −64.368 | 90.330 | 1.00 | 81.20 | | C |
| ANISOU | 1250 | CA | LEU | A | 180 | 8182 | 12135 | 10537 | 308 | 1825 | −1034 | C |
| ATOM | 1251 | C | LEU | A | 180 | 92.070 | −65.203 | 91.582 | 1.00 | 87.09 | | C |
| ANISOU | 1251 | C | LEU | A | 180 | 9157 | 12395 | 11537 | 52 | 1917 | −1076 | C |
| ATOM | 1252 | O | LEU | A | 180 | 92.611 | −66.312 | 91.493 | 1.00 | 87.68 | | O |
| ANISOU | 1252 | O | LEU | A | 180 | 9291 | 12247 | 11775 | −171 | 2050 | −1202 | O |
| ATOM | 1253 | CB | LEU | A | 180 | 93.136 | −64.001 | 89.634 | 1.00 | 80.75 | | C |
| ANISOU | 1253 | CB | LEU | A | 180 | 8243 | 11871 | 10568 | 441 | 1770 | −812 | C |
| ATOM | 1254 | CG | LEU | A | 180 | 93.103 | −63.971 | 88.108 | 1.00 | 84.99 | | C |
| ANISOU | 1254 | CG | LEU | A | 180 | 8526 | 12882 | 10885 | 566 | 1767 | −887 | C |
| ATOM | 1255 | CD1 | LEU | A | 180 | 94.345 | −63.297 | 87.548 | 1.00 | 84.51 | | C |
| ANISOU | 1255 | CD1 | LEU | A | 180 | 8625 | 12572 | 10913 | 761 | 1736 | −595 | C |
| ATOM | 1256 | CD2 | LEU | A | 180 | 92.941 | −65.369 | 87.528 | 1.00 | 87.84 | | C |
| ANISOU | 1256 | CD2 | LEU | A | 180 | 8688 | 13437 | 11251 | 232 | 1875 | −1288 | C |
| ATOM | 1257 | N | GLY | A | 181 | 91.642 | −64.666 | 92.727 | 1.00 | 83.02 | | N |
| ANISOU | 1257 | N | GLY | A | 181 | 8774 | 11735 | 11035 | 122 | 1882 | −957 | N |
| ATOM | 1258 | CA | GLY | A | 181 | 91.767 | −65.315 | 94.024 | 1.00 | 82.95 | | C |
| ANISOU | 1258 | CA | GLY | A | 181 | 8983 | 11335 | 11200 | −32 | 1974 | −944 | C |
| ATOM | 1259 | C | GLY | A | 181 | 93.143 | −65.255 | 94.658 | 1.00 | 86.54 | | C |
| ANISOU | 1259 | C | GLY | A | 181 | 9696 | 11360 | 11825 | 35 | 1912 | −698 | C |
| ATOM | 1260 | O | GLY | A | 181 | 93.480 | −66.138 | 95.458 | 1.00 | 86.95 | | O |
| ANISOU | 1260 | O | GLY | A | 181 | 9906 | 11134 | 11998 | −58 | 2033 | −680 | O |
| ATOM | 1261 | N | MET | A | 182 | 93.913 | −64.218 | 94.305 | 1.00 | 81.20 | | N |
| ANISOU | 1261 | N | MET | A | 182 | 9054 | 10652 | 11148 | 215 | 1762 | −513 | N |
| ATOM | 1262 | CA | MET | A | 182 | 95.283 | −63.999 | 94.845 | 1.00 | 79.67 | | C |
| ANISOU | 1262 | CA | MET | A | 182 | 9033 | 10152 | 11086 | 264 | 1691 | −346 | C |
| ATOM | 1263 | C | MET | A | 182 | 95.226 | −62.888 | 95.899 | 1.00 | 81.77 | | C |
| ANISOU | 1263 | C | MET | A | 182 | 9397 | 10339 | 11334 | 367 | 1608 | −246 | C |
| ATOM | 1264 | O | MET | A | 182 | 95.811 | −61.813 | 95.664 | 1.00 | 82.66 | | O |
| ANISOU | 1264 | O | MET | A | 182 | 9535 | 10390 | 11483 | 455 | 1559 | −166 | O |
| ATOM | 1265 | CB | MET | A | 182 | 96.252 | −63.592 | 93.730 | 1.00 | 81.49 | | C |
| ANISOU | 1265 | CB | MET | A | 182 | 9236 | 10341 | 11386 | 309 | 1647 | −279 | C |
| ATOM | 1266 | CG | MET | A | 182 | 96.701 | −64.755 | 92.866 | 1.00 | 84.52 | | C |
| ANISOU | 1266 | CG | MET | A | 182 | 9457 | 10993 | 11663 | 372 | 1663 | −324 | C |
| ATOM | 1267 | SD | MET | A | 182 | 95.520 | −65.140 | 91.549 | 1.00 | 87.95 | | S |
| ANISOU | 1267 | SD | MET | A | 182 | 9751 | 11568 | 12099 | 175 | 1785 | −552 | S |
| ATOM | 1268 | CE | MET | A | 182 | 96.000 | −63.935 | 90.313 | 1.00 | 84.76 | | C |
| ANISOU | 1268 | CE | MET | A | 182 | 9275 | 11245 | 11685 | 297 | 1737 | −460 | C |
| ATOM | 1269 | N | GLY | A | 183 | 94.545 | −63.160 | 97.015 | 1.00 | 75.36 | | N |
| ANISOU | 1269 | N | GLY | A | 183 | 8644 | 9513 | 10478 | 333 | 1638 | −277 | N |
| ATOM | 1270 | CA | GLY | A | 183 | 94.361 | −62.220 | 98.118 | 1.00 | 73.89 | | C |
| ANISOU | 1270 | CA | GLY | A | 183 | 8542 | 9278 | 10255 | 403 | 1583 | −230 | C |
| ATOM | 1271 | C | GLY | A | 183 | 95.620 | −61.659 | 98.752 | 1.00 | 73.88 | | C |
| ANISOU | 1271 | C | GLY | A | 183 | 8613 | 9146 | 10314 | 431 | 1499 | −181 | C |
| ATOM | 1272 | O | GLY | A | 183 | 95.618 | −60.506 | 99.196 | 1.00 | 73.72 | | O |
| ANISOU | 1272 | O | GLY | A | 183 | 8623 | 9095 | 10293 | 464 | 1489 | −199 | O |
| ATOM | 1273 | N | GLN | A | 184 | 96.708 | −62.426 | 98.760 | 1.00 | 67.10 | | N |
| ANISOU | 1273 | N | GLN | A | 184 | 7763 | 8231 | 9499 | 417 | 1471 | −149 | N |
| ATOM | 1274 | CA | GLN | A | 184 | 97.963 | −61.927 | 99.384 | 1.00 | 66.32 | | C |
| ANISOU | 1274 | CA | GLN | A | 184 | 7655 | 8128 | 9415 | 442 | 1380 | −154 | C |
| ATOM | 1275 | C | GLN | A | 184 | 98.657 | −60.970 | 98.421 | 1.00 | 70.17 | | C |
| ANISOU | 1275 | C | GLN | A | 184 | 8087 | 8563 | 10014 | 407 | 1380 | −200 | C |
| ATOM | 1276 | O | GLN | A | 184 | 99.440 | −60.146 | 98.843 | 1.00 | 69.98 | | O |
| ANISOU | 1276 | O | GLN | A | 184 | 8035 | 8534 | 10022 | 363 | 1364 | −301 | O |
| ATOM | 1277 | CB | GLN | A | 184 | 98.885 | −63.071 | 99.760 | 1.00 | 67.79 | | C |
| ANISOU | 1277 | CB | GLN | A | 184 | 7847 | 8331 | 9580 | 509 | 1377 | −70 | C |
| ATOM | 1278 | CG | GLN | A | 184 | 98.203 | −64.416 | 99.707 | 1.00 | 91.58 | | C |
| ANISOU | 1278 | CG | GLN | A | 184 | 10945 | 11254 | 12596 | 517 | 1540 | 6 | C |
| ATOM | 1279 | CD | GLN | A | 184 | 98.314 | −65.019 | 98.332 | 1.00 | 118.12 | | C |
| ANISOU | 1279 | CD | GLN | A | 184 | 14263 | 14546 | 16073 | 425 | 1625 | −29 | C |
| ATOM | 1280 | OE1 | GLN | A | 184 | 98.887 | −66.090 | 98.152 | 1.00 | 113.87 | | O |
| ANISOU | 1280 | OE1 | GLN | A | 184 | 13644 | 14067 | 15553 | 352 | 1603 | −112 | O |
| ATOM | 1281 | NE2 | GLN | A | 184 | 97.750 | −64.331 | 97.352 | 1.00 | 109.62 | | N |
| ANISOU | 1281 | NE2 | GLN | A | 184 | 13233 | 13367 | 15052 | 460 | 1751 | 43 | N |
| ATOM | 1282 | N | LEU | A | 185 | 98.439 | −61.176 | 97.145 | 1.00 | 65.13 | | N |
| ANISOU | 1282 | N | LEU | A | 185 | 7422 | 7900 | 9425 | 417 | 1437 | −150 | N |
| ATOM | 1283 | CA | LEU | A | 185 | 98.919 | −60.251 | 96.125 | 1.00 | 63.12 | | C |
| ANISOU | 1283 | CA | LEU | A | 185 | 7146 | 7576 | 9261 | 437 | 1504 | −136 | C |
| ATOM | 1284 | C | LEU | A | 185 | 98.065 | −58.950 | 96.265 | 1.00 | 62.07 | | C |
| ANISOU | 1284 | C | LEU | A | 185 | 7076 | 7397 | 9112 | 509 | 1622 | −123 | C |
| ATOM | 1285 | O | LEU | A | 185 | 98.619 | −57.871 | 96.468 | 1.00 | 60.14 | | O |
| ANISOU | 1285 | O | LEU | A | 185 | 6879 | 7002 | 8970 | 479 | 1732 | −177 | O |
| ATOM | 1286 | CB | LEU | A | 185 | 98.755 | −60.904 | 94.721 | 1.00 | 63.05 | | C |
| ANISOU | 1286 | CB | LEU | A | 185 | 7074 | 7642 | 9241 | 475 | 1533 | −76 | C |
| ATOM | 1287 | CG | LEU | A | 185 | 99.925 | −60.780 | 93.725 | 1.00 | 67.47 | | C |
| ANISOU | 1287 | CG | LEU | A | 185 | 7599 | 8130 | 9907 | 471 | 1560 | −48 | C |
| ATOM | 1288 | CD1 | LEU | A | 185 | 101.120 | −61.550 | 94.208 | 1.00 | 67.24 | | C |
| ANISOU | 1288 | CD1 | LEU | A | 185 | 7546 | 8060 | 9944 | 392 | 1475 | −89 | C |
| ATOM | 1289 | CD2 | LEU | A | 185 | 99.538 | −61.305 | 92.330 | 1.00 | 69.38 | | C |
| ANISOU | 1289 | CD2 | LEU | A | 185 | 7763 | 8514 | 10086 | 528 | 1599 | −9 | C |
| ATOM | 1290 | N | LYS | A | 186 | 96.720 | −59.093 | 96.258 | 1.00 | 56.18 | | N |
| ANISOU | 1290 | N | LYS | A | 186 | 6323 | 6782 | 8241 | 594 | 1634 | −81 | N |
| ATOM | 1291 | CA | LYS | A | 186 | 95.730 | −58.026 | 96.363 | 1.00 | 54.85 | | C |
| ANISOU | 1291 | CA | LYS | A | 186 | 6203 | 6621 | 8015 | 730 | 1757 | −28 | C |
| ATOM | 1292 | C | LYS | A | 186 | 95.978 | −57.089 | 97.531 | 1.00 | 59.63 | | C |
| ANISOU | 1292 | C | LYS | A | 186 | 6916 | 7040 | 8701 | 662 | 1826 | −112 | C |
| ATOM | 1293 | O | LYS | A | 186 | 95.947 | −55.871 | 97.327 | 1.00 | 58.07 | | O |
| ANISOU | 1293 | O | LYS | A | 186 | 6807 | 6679 | 8578 | 749 | 2040 | −72 | O |
| ATOM | 1294 | CB | LYS | A | 186 | 94.312 | −58.611 | 96.438 | 1.00 | 55.84 | | C |
| ANISOU | 1294 | CB | LYS | A | 186 | 6249 | 6996 | 7971 | 786 | 1716 | −33 | C |
| ATOM | 1295 | CG | LYS | A | 186 | 93.194 | −57.575 | 96.329 | 1.00 | 64.83 | | C |
| ANISOU | 1295 | CG | LYS | A | 186 | 7395 | 8236 | 9001 | 1006 | 1845 | 58 | C |
| ATOM | 1296 | CD | LYS | A | 186 | 92.038 | −57.884 | 97.275 | 1.00 | 71.65 | | C |
| ANISOU | 1296 | CD | LYS | A | 186 | 8232 | 9236 | 9758 | 971 | 1800 | −20 | C |
| ATOM | 1297 | CE | LYS | A | 186 | 90.934 | −56.857 | 97.285 | 1.00 | 71.58 | | C |
| ANISOU | 1297 | CE | LYS | A | 186 | 8223 | 9342 | 9631 | 1216 | 1933 | 78 | C |
| ATOM | 1298 | NZ | LYS | A | 186 | 89.785 | −57.347 | 98.095 | 1.00 | 80.72 | | N |
| ANISOU | 1298 | NZ | LYS | A | 186 | 9310 | 10684 | 10674 | 1155 | 1876 | −28 | N |
| ATOM | 1299 | N | TRP | A | 187 | 96.203 | −57.631 | 98.752 | 1.00 | 58.28 | | N |
| ANISOU | 1299 | N | TRP | A | 187 | 6740 | 6902 | 8502 | 527 | 1691 | −231 | N |
| ATOM | 1300 | CA | TRP | A | 187 | 96.426 | −56.756 | 99.909 | 1.00 | 59.02 | | C |
| ANISOU | 1300 | CA | TRP | A | 187 | 6890 | 6906 | 8631 | 441 | 1748 | −376 | C |
| ATOM | 1301 | C | TRP | A | 187 | 97.721 | −55.957 | 99.748 | 1.00 | 59.09 | | C |
| ANISOU | 1301 | C | TRP | A | 187 | 6887 | 6764 | 8802 | 316 | 1862 | −518 | C |
| ATOM | 1302 | O | TRP | A | 187 | 97.705 | −54.751 | 100.000 | 1.00 | 57.56 | | O |
| ANISOU | 1302 | O | TRP | A | 187 | 6771 | 6386 | 8712 | 268 | 2090 | −623 | O |
| ATOM | 1303 | CB | TRP | A | 187 | 96.314 | −57.465 | 101.291 | 1.00 | 59.55 | | C |
| ANISOU | 1303 | CB | TRP | A | 187 | 6934 | 7125 | 8566 | 381 | 1589 | −460 | C |
| ATOM | 1304 | CG | TRP | A | 187 | 97.527 | −58.222 | 101.748 | 1.00 | 62.15 | | C |
| ANISOU | 1304 | CG | TRP | A | 187 | 7172 | 7584 | 8856 | 320 | 1443 | −529 | C |
| ATOM | 1305 | CD1 | TRP | A | 187 | 97.684 | −59.576 | 101.764 | 1.00 | 65.40 | | C |
| ANISOU | 1305 | CD1 | TRP | A | 187 | 7562 | 8100 | 9186 | 387 | 1339 | −413 | C |
| ATOM | 1306 | CD2 | TRP | A | 187 | 98.758 | −57.669 | 102.255 | 1.00 | 62.42 | | C |
| ANISOU | 1306 | CD2 | TRP | A | 187 | 7108 | 7693 | 8917 | 204 | 1426 | −742 | C |
| ATOM | 1307 | NE1 | TRP | A | 187 | 98.963 | −59.902 | 102.165 | 1.00 | 65.37 | | N |
| ANISOU | 1307 | NE1 | TRP | A | 187 | 7459 | 8242 | 9137 | 387 | 1244 | −476 | N |
| ATOM | 1308 | CE2 | TRP | A | 187 | 99.647 | −58.749 | 102.464 | 1.00 | 66.71 | | C |
| ANISOU | 1308 | CE2 | TRP | A | 187 | 7546 | 8445 | 9356 | 261 | 1268 | −704 | C |
| ATOM | 1309 | CE3 | TRP | A | 187 | 99.211 | −56.358 | 102.510 | 1.00 | 63.90 | | C |
| ANISOU | 1309 | CE3 | TRP | A | 187 | 7270 | 7795 | 9212 | 46 | 1577 | −992 | C |
| ATOM | 1310 | CZ2 | TRP | A | 187 | 100.945 | −58.567 | 102.958 | 1.00 | 65.98 | | C |
| ANISOU | 1310 | CZ2 | TRP | A | 187 | 7274 | 8585 | 9209 | 195 | 1195 | −909 | C |
| ATOM | 1311 | CZ3 | TRP | A | 187 | 100.512 | −56.174 | 102.943 | 1.00 | 65.62 | | C |
| ANISOU | 1311 | CZ3 | TRP | A | 187 | 7311 | 8202 | 9421 | −98 | 1535 | −1256 | C |
| ATOM | 1312 | CH2 | TRP | A | 187 | 101.352 | −57.271 | 103.199 | 1.00 | 66.37 | | C |
| ANISOU | 1312 | CH2 | TRP | A | 187 | 7251 | 8611 | 9354 | −10 | 1313 | −1217 | C |
| ATOM | 1313 | N | GLU | A | 188 | 98.825 | −56.611 | 99.279 | 1.00 | 53.99 | | N |
| ANISOU | 1313 | N | GLU | A | 188 | 6145 | 6175 | 8193 | 255 | 1755 | −536 | N |
| ATOM | 1314 | CA | GLU | A | 188 | 100.119 | −55.949 | 99.097 | 1.00 | 52.25 | | C |
| ANISOU | 1314 | CA | GLU | A | 188 | 5868 | 5857 | 8127 | 103 | 1866 | −716 | C |
| ATOM | 1315 | C | GLU | A | 188 | 100.097 | −54.917 | 98.016 | 1.00 | 51.68 | | C |
| ANISOU | 1315 | C | GLU | A | 188 | 5910 | 5497 | 8230 | 149 | 2174 | −638 | C |
| ATOM | 1316 | O | GLU | A | 188 | 100.674 | −53.851 | 98.197 | 1.00 | 50.97 | | O |
| ANISOU | 1316 | O | GLU | A | 188 | 5853 | 5207 | 8307 | −2 | 2434 | −835 | O |
| ATOM | 1317 | CB | GLU | A | 188 | 101.237 | −56.940 | 98.870 | 1.00 | 53.72 | | C |
| ANISOU | 1317 | CB | GLU | A | 188 | 5911 | 6212 | 8287 | 68 | 1679 | −734 | C |
| ATOM | 1318 | CG | GLU | A | 188 | 102.559 | −56.410 | 99.389 | 1.00 | 62.64 | | C |
| ANISOU | 1318 | CG | GLU | A | 188 | 6887 | 7430 | 9482 | −133 | 1711 | −1043 | C |
| ATOM | 1319 | CD | GLU | A | 188 | 103.656 | −57.446 | 99.495 | 1.00 | 74.93 | | C |
| ANISOU | 1319 | CD | GLU | A | 188 | 8256 | 9274 | 10938 | −112 | 1491 | −1067 | C |
| ATOM | 1320 | OE1 | GLU | A | 188 | 103.346 | −58.648 | 99.678 | 1.00 | 85.20 | | O |
| ANISOU | 1320 | OE1 | GLU | A | 188 | 9571 | 10717 | 12085 | 63 | 1314 | −861 | O |
| ATOM | 1321 | OE2 | GLU | A | 188 | 104.836 | −57.043 | 99.418 | 1.00 | 46.40 | | O |
| ANISOU | 1321 | OE2 | GLU | A | 188 | 4481 | 5744 | 7404 | −266 | 1540 | −1303 | O |
| ATOM | 1322 | N | LEU | A | 189 | 99.410 | −55.210 | 96.911 | 1.00 | 46.22 | | N |
| ANISOU | 1322 | N | LEU | A | 189 | 5271 | 4802 | 7487 | 364 | 2191 | −368 | N |
| ATOM | 1323 | CA | LEU | A | 189 | 99.250 | −54.257 | 95.823 | 1.00 | 45.87 | | C |
| ANISOU | 1323 | CA | LEU | A | 189 | 5348 | 4539 | 7541 | 528 | 2511 | −204 | C |
| ATOM | 1324 | C | LEU | A | 189 | 98.515 | −53.015 | 96.349 | 1.00 | 51.05 | | C |
| ANISOU | 1324 | C | LEU | A | 189 | 6164 | 4980 | 8252 | 598 | 2818 | −205 | C |
| ATOM | 1325 | O | LEU | A | 189 | 99.002 | −51.908 | 96.126 | 1.00 | 50.40 | | O |
| ANISOU | 1325 | O | LEU | A | 189 | 6209 | 4569 | 8371 | 562 | 3205 | −252 | O |
| ATOM | 1326 | CB | LEU | A | 189 | 98.506 | −54.885 | 94.628 | 1.00 | 45.69 | | C |
| ANISOU | 1326 | CB | LEU | A | 189 | 5289 | 4717 | 7354 | 792 | 2430 | 65 | C |
| ATOM | 1327 | CG | LEU | A | 189 | 99.281 | −55.893 | 93.793 | 1.00 | 50.21 | | C |
| ANISOU | 1327 | CG | LEU | A | 189 | 5744 | 5415 | 7919 | 742 | 2257 | 79 | C |
| ATOM | 1328 | CD1 | LEU | A | 189 | 98.351 | −56.901 | 93.128 | 1.00 | 50.13 | | C |
| ANISOU | 1328 | CD1 | LEU | A | 189 | 5625 | 5724 | 7696 | 885 | 2084 | 192 | C |
| ATOM | 1329 | CD2 | LEU | A | 189 | 100.145 | −55.208 | 92.777 | 1.00 | 52.25 | | C |
| ANISOU | 1329 | CD2 | LEU | A | 189 | 6061 | 5469 | 8322 | 789 | 2507 | 153 | C |
| ATOM | 1330 | N | GLU | A | 190 | 97.406 | −53.210 | 97.120 | 1.00 | 49.02 | | N |
| ANISOU | 1330 | N | GLU | A | 190 | 5908 | 4876 | 7841 | 671 | 2689 | −182 | N |
| ATOM | 1331 | CA | GLU | A | 190 | 96.597 | −52.136 | 97.705 | 1.00 | 49.80 | | C |
| ANISOU | 1331 | CA | GLU | A | 190 | 6154 | 4797 | 7970 | 759 | 2962 | −175 | C |
| ATOM | 1332 | C | GLU | A | 190 | 97.376 | −51.276 | 98.676 | 1.00 | 56.18 | | C |
| ANISOU | 1332 | C | GLU | A | 190 | 7016 | 5348 | 8980 | 458 | 3178 | −508 | C |
| ATOM | 1333 | O | GLU | A | 190 | 97.371 | −50.049 | 98.528 | 1.00 | 55.13 | | O |
| ANISOU | 1333 | O | GLU | A | 190 | 7063 | 4855 | 9031 | 491 | 3642 | −514 | O |
| ATOM | 1334 | CB | GLU | A | 190 | 95.326 | −52.680 | 98.371 | 1.00 | 51.22 | | C |
| ANISOU | 1334 | CB | GLU | A | 190 | 6285 | 5240 | 7935 | 861 | 2739 | −119 | C |
| ATOM | 1335 | CG | GLU | A | 190 | 94.234 | −53.011 | 97.366 | 1.00 | 63.45 | | C |
| ANISOU | 1335 | CG | GLU | A | 190 | 7783 | 7039 | 9286 | 1197 | 2702 | 167 | C |
| ATOM | 1336 | CD | GLU | A | 190 | 92.851 | −53.413 | 97.842 | 1.00 | 87.92 | | C |
| ANISOU | 1336 | CD | GLU | A | 190 | 10809 | 10421 | 12176 | 1316 | 2560 | 208 | C |
| ATOM | 1337 | OE1 | GLU | A | 190 | 92.679 | −53.747 | 99.039 | 1.00 | 76.37 | | O |
| ANISOU | 1337 | OE1 | GLU | A | 190 | 9341 | 8973 | 10703 | 1123 | 2417 | 38 | O |
| ATOM | 1338 | OE2 | GLU | A | 190 | 91.934 | −53.411 | 96.989 | 1.00 | 85.84 | | O |
| ANISOU | 1338 | OE2 | GLU | A | 190 | 10468 | 10417 | 11731 | 1617 | 2599 | 402 | O |
| ATOM | 1339 | N | ASP | A | 191 | 98.083 | −51.922 | 99.632 | 1.00 | 55.22 | | N |
| ANISOU | 1339 | N | ASP | A | 191 | 6730 | 5435 | 8816 | 178 | 2887 | −796 | N |
| ATOM | 1340 | CA | ASP | A | 191 | 98.865 | −51.265 | 100.687 | 1.00 | 56.56 | | C |
| ANISOU | 1340 | CA | ASP | A | 191 | 6846 | 5545 | 9100 | −147 | 3016 | −1215 | C |
| ATOM | 1341 | C | ASP | A | 191 | 100.068 | −50.454 | 100.173 | 1.00 | 59.62 | | C |
| ANISOU | 1341 | C | ASP | A | 191 | 7237 | 5661 | 9756 | −368 | 3366 | −1441 | C |
| ATOM | 1342 | O | ASP | A | 191 | 100.376 | −49.398 | 100.726 | 1.00 | 57.47 | | O |
| ANISOU | 1342 | O | ASP | A | 191 | 7010 | 5162 | 9664 | −609 | 3732 | −1772 | O |
| ATOM | 1343 | CB | ASP | A | 191 | 99.285 | −52.282 | 101.766 | 1.00 | 59.78 | | C |
| ANISOU | 1343 | CB | ASP | A | 191 | 7030 | 6385 | 9297 | −284 | 2588 | −1411 | C |
| ATOM | 1344 | CG | ASP | A | 191 | 98.268 | −52.456 | 102.901 | 1.00 | 85.13 | | C |
| ANISOU | 1344 | CG | ASP | A | 191 | 10264 | 9759 | 12323 | −224 | 2451 | −1414 | C |
| ATOM | 1345 | OD1 | ASP | A | 191 | 97.091 | −52.025 | 102.732 | 1.00 | 87.01 | | O |
| ANISOU | 1345 | OD1 | ASP | A | 191 | 10671 | 9820 | 12569 | −47 | 2605 | −1214 | O |
| ATOM | 1346 | OD2 | ASP | A | 191 | 98.646 | −53.019 | 103.957 | 1.00 | 97.34 | | O |
| ANISOU | 1346 | OD2 | ASP | A | 191 | 11650 | 11644 | 13692 | −319 | 2202 | −1602 | O |
| ATOM | 1347 | N | LEU | A | 192 | 100.747 | −50.943 | 99.125 | 1.00 | 57.75 | | N |
| ANISOU | 1347 | N | LEU | A | 192 | 6948 | 5436 | 9557 | −314 | 3295 | −1298 | N |
| ATOM | 1348 | CA | LEU | A | 192 | 101.869 | −50.219 | 98.534 | 1.00 | 56.94 | | C |
| ANISOU | 1348 | CA | LEU | A | 192 | 6855 | 5060 | 9721 | −515 | 3656 | −1490 | C |
| ATOM | 1349 | CB | LEU | A | 192 | 102.779 | −51.130 | 97.730 | 1.00 | 55.39 | | C |
| ANISOU | 1349 | CB | LEU | A | 192 | 6502 | 5052 | 9491 | −510 | 3400 | −1413 | C |
| ATOM | 1350 | CG | LEU | A | 192 | 103.742 | −51.958 | 98.581 | 1.00 | 58.86 | | C |
| ANISOU | 1350 | CG | LEU | A | 192 | 6631 | 5937 | 9798 | −737 | 3011 | −1732 | C |
| ATOM | 1351 | CD1 | LEU | A | 192 | 104.083 | −53.283 | 97.916 | 1.00 | 59.48 | | C |
| ANISOU | 1351 | CD1 | LEU | A | 192 | 6599 | 6267 | 9733 | −569 | 2637 | −1483 | C |
| ATOM | 1352 | CD2 | LEU | A | 192 | 104.998 | −51.188 | 98.918 | 1.00 | 59.01 | | C |
| ANISOU | 1352 | CD2 | LEU | A | 192 | 6484 | 5929 | 10009 | −1127 | 3272 | −2240 | C |
| ATOM | 1353 | C | LEU | A | 192 | 101.342 | −49.056 | 97.715 | 1.00 | 65.50 | | C |
| ANISOU | 1353 | C | LEU | A | 192 | 8244 | 5635 | 11008 | −323 | 4233 | −1258 | C |
| ATOM | 1354 | O | LEU | A | 192 | 101.916 | −47.975 | 97.771 | 1.00 | 66.89 | | O |
| ANISOU | 1354 | O | LEU | A | 192 | 8519 | 5432 | 11464 | −551 | 4749 | −1521 | O |
| ATOM | 1355 | N | SER | A | 193 | 100.196 | −49.243 | 97.038 | 1.00 | 63.13 | | N |
| ANISOU | 1355 | N | SER | A | 193 | 8086 | 5349 | 10551 | 107 | 4192 | −789 | N |
| ATOM | 1356 | CA | SER | A | 193 | 99.531 | −48.194 | 96.270 | 1.00 | 63.60 | | C |
| ANISOU | 1356 | CA | SER | A | 193 | 8433 | 5022 | 10711 | 440 | 4731 | −467 | C |
| ATOM | 1357 | CB | SER | A | 193 | 98.322 | −48.761 | 95.553 | 1.00 | 64.12 | | C |
| ANISOU | 1357 | CB | SER | A | 193 | 8504 | 5382 | 10475 | 926 | 4497 | 3 | C |
| ATOM | 1358 | OG | SER | A | 193 | 98.698 | −49.533 | 94.434 | 1.00 | 66.58 | | O |
| ANISOU | 1358 | OG | SER | A | 193 | 8709 | 5906 | 10683 | 1058 | 4282 | 196 | O |
| ATOM | 1359 | C | SER | A | 193 | 99.068 | −47.071 | 97.192 | 1.00 | 74.24 | | C |
| ANISOU | 1359 | C | SER | A | 193 | 9958 | 6041 | 12211 | 348 | 5161 | −647 | C |
| ATOM | 1360 | O | SER | A | 193 | 99.207 | −45.893 | 96.852 | 1.00 | 73.90 | | O |
| ANISOU | 1360 | O | SER | A | 193 | 10167 | 5485 | 12427 | 391 | 5828 | −619 | O |
| ATOM | 1361 | N | PHE | A | 194 | 98.507 | −47.449 | 98.364 | 1.00 | 75.37 | | N |
| ANISOU | 1361 | N | PHE | A | 194 | 9986 | 6456 | 12197 | 230 | 4822 | −827 | N |
| ATOM | 1362 | CA | PHE | A | 194 | 98.023 | −46.532 | 99.392 | 1.00 | 76.62 | | C |
| ANISOU | 1362 | CA | PHE | A | 194 | 10272 | 6382 | 12460 | 109 | 5147 | −1047 | C |
| ATOM | 1363 | C | PHE | A | 194 | 99.178 | −45.648 | 99.883 | 1.00 | 82.93 | | C |
| ANISOU | 1363 | C | PHE | A | 194 | 11074 | 6849 | 13588 | −384 | 5593 | −1590 | C |
| ATOM | 1364 | O | PHE | A | 194 | 99.010 | −44.440 | 99.969 | 1.00 | 82.95 | | O |
| ANISOU | 1364 | O | PHE | A | 194 | 11326 | 6345 | 13845 | −415 | 6253 | −1670 | O |
| ATOM | 1365 | CB | PHE | A | 194 | 97.408 | −47.347 | 100.544 | 1.00 | 78.95 | | C |
| ANISOU | 1365 | CB | PHE | A | 194 | 10383 | 7127 | 12487 | 38 | 4606 | −1169 | C |
| ATOM | 1366 | CG | PHE | A | 194 | 96.824 | −46.603 | 101.727 | 1.00 | 81.37 | | C |
| ANISOU | 1366 | CG | PHE | A | 194 | 10775 | 7308 | 12833 | −87 | 4823 | −1409 | C |
| ATOM | 1367 | CD1 | PHE | A | 194 | 96.360 | −45.302 | 101.596 | 1.00 | 84.73 | | C |
| ANISOU | 1367 | CD1 | PHE | A | 194 | 11494 | 7220 | 13479 | 35 | 5485 | −1340 | C |
| ATOM | 1368 | CD2 | PHE | A | 194 | 96.676 | −47.235 | 102.958 | 1.00 | 84.09 | | C |
| ANISOU | 1368 | CD2 | PHE | A | 194 | 10927 | 8049 | 12975 | −275 | 4394 | −1663 | C |
| ATOM | 1369 | CE1 | PHE | A | 194 | 95.828 | −44.620 | 102.687 | 1.00 | 85.89 | | C |
| ANISOU | 1369 | CE1 | PHE | A | 194 | 11727 | 7233 | 13675 | −95 | 5714 | −1584 | C |
| ATOM | 1370 | CE2 | PHE | A | 194 | 96.136 | −46.553 | 104.049 | 1.00 | 86.97 | | C |
| ANISOU | 1370 | CE2 | PHE | A | 194 | 11361 | 8323 | 13359 | −393 | 4592 | −1901 | C |
| ATOM | 1371 | CZ | PHE | A | 194 | 95.719 | −45.248 | 103.906 | 1.00 | 85.16 | | C |
| ANISOU | 1371 | CZ | PHE | A | 194 | 11413 | 7568 | 13376 | −324 | 5245 | −1881 | C |
| ATOM | 1372 | N | ARG | A | 195 | 100.359 | −46.246 | 100.128 | 1.00 | 80.87 | | N |
| ANISOU | 1372 | N | ARG | A | 195 | 10527 | 6877 | 13323 | −753 | 5284 | −1963 | N |
| ATOM | 1373 | CA | ARG | A | 195 | 101.562 | −45.574 | 100.593 | 1.00 | 81.08 | | C |
| ANISOU | 1373 | CA | ARG | A | 195 | 10436 | 6762 | 13610 | −1276 | 5626 | −2577 | C |
| ATOM | 1374 | CB | ARG | A | 195 | 102.725 | −46.577 | 100.671 | 1.00 | 79.30 | | C |
| ANISOU | 1374 | CB | ARG | A | 195 | 9832 | 7053 | 13247 | −1510 | 5112 | −2832 | C |
| ATOM | 1375 | CG | ARG | A | 195 | 104.132 | −45.969 | 100.829 | 1.00 | 85.82 | | C |
| ANISOU | 1375 | CG | ARG | A | 195 | 10463 | 7817 | 14328 | −2035 | 5458 | −3465 | C |
| ATOM | 1376 | CD | ARG | A | 195 | 105.230 | −47.006 | 100.679 | 1.00 | 88.81 | | C |
| ANISOU | 1376 | CD | ARG | A | 195 | 10476 | 8727 | 14539 | −2139 | 4952 | −3593 | C |
| ATOM | 1377 | NE | ARG | A | 195 | 105.063 | −48.103 | 101.637 | 1.00 | 97.28 | | N |
| ANISOU | 1377 | NE | ARG | A | 195 | 11276 | 10478 | 15208 | −2047 | 4266 | −3608 | N |
| ATOM | 1378 | CZ | ARG | A | 195 | 105.763 | −48.236 | 102.755 | 1.00 | 110.19 | | C |
| ANISOU | 1378 | CZ | ARG | A | 195 | 12539 | 12655 | 16674 | −2349 | 4058 | −4152 | C |
| ATOM | 1379 | NH1 | ARG | A | 195 | 106.720 | −47.365 | 103.055 | 1.00 | 99.33 | | N |
| ANISOU | 1379 | NH1 | ARG | A | 195 | 10965 | 11272 | 15503 | −2833 | 4453 | −4814 | N |
| ATOM | 1380 | NH2 | ARG | A | 195 | 105.523 | −49.251 | 103.577 | 1.00 | 91.08 | | N |
| ANISOU | 1380 | NH2 | ARG | A | 195 | 9925 | 10820 | 13861 | −2159 | 3486 | −4056 | N |
| ATOM | 1381 | C | ARG | A | 195 | 101.937 | −44.429 | 99.700 | 1.00 | 89.33 | | C |
| ANISOU | 1381 | C | ARG | A | 195 | 11763 | 7138 | 15042 | −1303 | 6422 | −2550 | C |
| ATOM | 1382 | O | ARG | A | 195 | 102.214 | −43.349 | 100.202 | 1.00 | 88.98 | | O |
| ANISOU | 1382 | O | ARG | A | 195 | 11815 | 6706 | 15287 | −1642 | 7028 | −2982 | O |
| ATOM | 1383 | N | TYR | A | 196 | 101.968 | −44.660 | 98.382 | 1.00 | 90.38 | | N |
| ANISOU | 1383 | N | TYR | A | 196 | 12028 | 7130 | 15183 | −956 | 6469 | −2065 | N |
| ATOM | 1384 | CA | TYR | A | 196 | 102.425 | −43.650 | 97.420 | 1.00 | 92.14 | | C |
| ANISOU | 1384 | CA | TYR | A | 196 | 12534 | 6716 | 15759 | −926 | 7256 | −1975 | C |
| ATOM | 1385 | CB | TYR | A | 196 | 103.178 | −44.297 | 96.226 | 1.00 | 92.43 | | C |
| ANISOU | 1385 | CB | TYR | A | 196 | 12486 | 6868 | 15767 | −808 | 7066 | −1737 | C |
| ATOM | 1386 | CG | TYR | A | 196 | 104.426 | −45.066 | 96.640 | 1.00 | 93.41 | | C |
| ANISOU | 1386 | CG | TYR | A | 196 | 12196 | 7437 | 15859 | −1282 | 6602 | −2242 | C |
| ATOM | 1387 | CD1 | TYR | A | 196 | 105.596 | −44.402 | 97.001 | 1.00 | 94.88 | | C |
| ANISOU | 1387 | CD1 | TYR | A | 196 | 12255 | 7441 | 16355 | −1844 | 7022 | −2882 | C |
| ATOM | 1388 | CE1 | TYR | A | 196 | 106.741 | −45.104 | 97.390 | 1.00 | 96.05 | | C |
| ANISOU | 1388 | CE1 | TYR | A | 196 | 11969 | 8108 | 16418 | −2226 | 6584 | −3346 | C |
| ATOM | 1389 | CZ | TYR | A | 196 | 106.729 | −46.491 | 97.400 | 1.00 | 105.20 | | C |
| ANISOU | 1389 | CZ | TYR | A | 196 | 12877 | 9888 | 17205 | −2011 | 5765 | −3111 | C |
| ATOM | 1390 | OH | TYR | A | 196 | 107.853 | −47.201 | 97.764 | 1.00 | 108.23 | | O |
| ANISOU | 1390 | OH | TYR | A | 196 | 12842 | 10808 | 17474 | −2295 | 5360 | −3503 | O |
| ATOM | 1391 | CE2 | TYR | A | 196 | 105.578 | −47.171 | 97.035 | 1.00 | 95.97 | | C |
| ANISOU | 1391 | CE2 | TYR | A | 196 | 11872 | 8817 | 15776 | −1498 | 5395 | −2493 | C |
| ATOM | 1392 | CD2 | TYR | A | 196 | 104.435 | −46.458 | 96.668 | 1.00 | 94.58 | | C |
| ANISOU | 1392 | CD2 | TYR | A | 196 | 12068 | 8207 | 15662 | −1156 | 5788 | −2093 | C |
| ATOM | 1393 | C | TYR | A | 196 | 101.306 | −42.683 | 96.966 | 1.00 | 100.54 | | C |
| ANISOU | 1393 | C | TYR | A | 196 | 14038 | 7254 | 16909 | −438 | 7886 | −1484 | C |
| ATOM | 1394 | O | TYR | A | 196 | 101.607 | −41.513 | 96.710 | 1.00 | 101.37 | | O |
| ANISOU | 1394 | O | TYR | A | 196 | 14434 | 6699 | 17383 | −523 | 8752 | −1573 | O |
| ATOM | 1395 | N | LEU | A | 197 | 100.030 | −43.134 | 96.951 | 1.00 | 98.16 | | N |
| ANISOU | 1395 | N | LEU | A | 197 | 13776 | 7246 | 16275 | 55 | 7509 | −1012 | N |
| ATOM | 1396 | CA | LEU | A | 197 | 98.890 | −42.298 | 96.562 | 1.00 | 98.55 | | C |
| ANISOU | 1396 | CA | LEU | A | 197 | 14186 | 6940 | 16318 | 606 | 8038 | −511 | C |
| ATOM | 1397 | CB | LEU | A | 197 | 97.670 | −43.177 | 96.231 | 1.00 | 98.20 | | C |
| ANISOU | 1397 | CB | LEU | A | 197 | 14034 | 7464 | 15812 | 1156 | 7425 | 7 | C |
| ATOM | 1398 | CG | LEU | A | 197 | 96.672 | −42.626 | 95.220 | 1.00 | 102.19 | | C |
| ANISOU | 1398 | CG | LEU | A | 197 | 14808 | 7845 | 16173 | 1912 | 7839 | 687 | C |
| ATOM | 1399 | CD1 | LEU | A | 197 | 97.204 | −42.714 | 93.810 | 1.00 | 101.91 | | C |
| ANISOU | 1399 | CD1 | LEU | A | 197 | 14829 | 7775 | 16117 | 2207 | 8001 | 1038 | C |
| ATOM | 1400 | CD2 | LEU | A | 197 | 95.379 | −43.395 | 95.279 | 1.00 | 104.42 | | C |
| ANISOU | 1400 | CD2 | LEU | A | 197 | 14909 | 8756 | 16009 | 2314 | 7249 | 994 | C |
| ATOM | 1401 | C | LEU | A | 197 | 98.564 | −41.254 | 97.649 | 1.00 | 107.06 | | C |
| ANISOU | 1401 | C | LEU | A | 197 | 15448 | 7595 | 17635 | 369 | 8579 | −851 | C |
| ATOM | 1402 | O | LEU | A | 197 | 98.642 | −40.058 | 97.378 | 1.00 | 107.57 | | O |
| ANISOU | 1402 | O | LEU | A | 197 | 15868 | 6973 | 18033 | 433 | 9485 | −807 | O |
| ATOM | 1403 | N | HIS | A | 198 | 98.224 | −41.702 | 98.876 | 1.00 | 106.37 | | N |
| ANISOU | 1403 | N | HIS | A | 198 | 15134 | 7893 | 17387 | 95 | 8077 | −1195 | N |
| ATOM | 1404 | CA | HIS | A | 198 | 97.939 | −40.836 | 100.021 | 1.00 | 107.63 | | C |
| ANISOU | 1404 | CA | HIS | A | 198 | 15407 | 7755 | 17732 | −189 | 8495 | −1597 | C |
| ATOM | 1405 | CB | HIS | A | 198 | 96.521 | −41.060 | 100.582 | 1.00 | 109.21 | | C |
| ANISOU | 1405 | CB | HIS | A | 198 | 15632 | 8229 | 17633 | 185 | 8179 | −1297 | C |
| ATOM | 1406 | CG | HIS | A | 198 | 95.522 | −41.597 | 99.607 | 1.00 | 113.27 | | C |
| ANISOU | 1406 | CG | HIS | A | 198 | 16192 | 9027 | 17820 | 890 | 7887 | −564 | C |
| ATOM | 1407 | ND1 | HIS | A | 198 | 94.938 | −42.834 | 99.791 | 1.00 | 115.38 | | N |
| ANISOU | 1407 | ND1 | HIS | A | 198 | 16169 | 9978 | 17692 | 1014 | 7043 | −429 | N |
| ATOM | 1408 | CE1 | HIS | A | 198 | 94.102 | −42.993 | 98.777 | 1.00 | 115.05 | | C |
| ANISOU | 1408 | CE1 | HIS | A | 198 | 16195 | 10097 | 17422 | 1631 | 7017 | 166 | C |
| ATOM | 1409 | NE2 | HIS | A | 198 | 94.101 | −41.931 | 97.976 | 1.00 | 115.49 | | N |
| ANISOU | 1409 | NE2 | HIS | A | 198 | 16577 | 9636 | 17666 | 1979 | 7779 | 479 | N |
| ATOM | 1410 | CD2 | HIS | A | 198 | 95.012 | −41.036 | 98.487 | 1.00 | 115.66 | | C |
| ANISOU | 1410 | CD2 | HIS | A | 198 | 16772 | 9056 | 18118 | 1498 | 8373 | 26 | C |
| ATOM | 1411 | C | HIS | A | 198 | 98.985 | −41.113 | 101.113 | 1.00 | 113.81 | | C |
| ANISOU | 1411 | C | HIS | A | 198 | 15838 | 8823 | 18582 | −931 | 8208 | −2404 | C |
| ATOM | 1412 | O | HIS | A | 198 | 98.686 | −41.841 | 102.063 | 1.00 | 112.31 | | O |
| ANISOU | 1412 | O | HIS | A | 198 | 15385 | 9180 | 18109 | −1049 | 7569 | −2587 | O |
| ATOM | 1413 | N | PRO | A | 199 | 100.229 | −40.582 | 101.001 | 1.00 | 114.62 | | N |
| ANISOU | 1413 | N | PRO | A | 199 | 15898 | 8631 | 19022 | −1423 | 8668 | −2905 | N |
| ATOM | 1414 | CA | PRO | A | 199 | 101.239 | −40.880 | 102.036 | 1.00 | 115.61 | | C |
| ANISOU | 1414 | CA | PRO | A | 199 | 15599 | 9195 | 19130 | −2095 | 8353 | −3708 | C |
| ATOM | 1415 | CB | PRO | A | 199 | 102.548 | −40.320 | 101.452 | 1.00 | 117.48 | | C |
| ANISOU | 1415 | CB | PRO | A | 199 | 15818 | 9073 | 19746 | −2509 | 8920 | −4105 | C |
| ATOM | 1416 | CG | PRO | A | 199 | 102.126 | −39.285 | 100.485 | 1.00 | 121.99 | | C |
| ANISOU | 1416 | CG | PRO | A | 199 | 16915 | 8781 | 20655 | −2168 | 9832 | −3656 | C |
| ATOM | 1417 | CD | PRO | A | 199 | 100.785 | −39.706 | 99.943 | 1.00 | 117.32 | | C |
| ANISOU | 1417 | CD | PRO | A | 199 | 16547 | 8279 | 19751 | −1383 | 9510 | −2784 | C |
| ATOM | 1418 | C | PRO | A | 199 | 100.913 | −40.320 | 103.425 | 1.00 | 121.05 | | C |
| ANISOU | 1418 | C | PRO | A | 199 | 16230 | 9922 | 19843 | −2448 | 8516 | −4256 | C |
| ATOM | 1419 | O | PRO | A | 199 | 101.211 | −40.985 | 104.412 | 1.00 | 119.93 | | O |
| ANISOU | 1419 | O | PRO | A | 199 | 15692 | 10449 | 19426 | −2729 | 7910 | −4671 | O |
| ATOM | 1420 | N | GLU | A | 200 | 100.280 | −39.124 | 103.496 | 1.00 | 119.46 | | N |
| ANISOU | 1420 | N | GLU | A | 200 | 16426 | 9022 | 19941 | −2385 | 9347 | −4224 | N |
| ATOM | 1421 | CA | GLU | A | 200 | 99.891 | −38.452 | 104.738 | 1.00 | 119.94 | | C |
| ANISOU | 1421 | CA | GLU | A | 200 | 16494 | 9007 | 20072 | −2706 | 9636 | −4733 | C |
| ATOM | 1422 | CB | GLU | A | 200 | 99.268 | −37.085 | 104.440 | 1.00 | 121.84 | | C |
| ANISOU | 1422 | CB | GLU | A | 200 | 17274 | 8299 | 20721 | −2533 | 10725 | −4553 | C |
| ATOM | 1423 | CG | GLU | A | 200 | 99.316 | −36.146 | 105.634 | 1.00 | 138.64 | | C |
| ANISOU | 1423 | CG | GLU | A | 200 | 19404 | 10185 | 23089 | −3097 | 11299 | −5331 | C |
| ATOM | 1424 | CD | GLU | A | 200 | 100.588 | −35.328 | 105.776 | 1.00 | 169.89 | | C |
| ANISOU | 1424 | CD | GLU | A | 200 | 23166 | 14436 | 26946 | −3460 | 10924 | −5717 | C |
| ATOM | 1425 | OE1 | GLU | A | 200 | 101.667 | −35.787 | 105.327 | 1.00 | 162.77 | | O |
| ANISOU | 1425 | OE1 | GLU | A | 200 | 22050 | 13410 | 26384 | −3969 | 11381 | −6240 | O |
| ATOM | 1426 | OE2 | GLU | A | 200 | 100.498 | −34.216 | 106.347 | 1.00 | 169.58 | | O |
| ANISOU | 1426 | OE2 | GLU | A | 200 | 23242 | 14342 | 26847 | −3490 | 10904 | −5818 | O |
| ATOM | 1427 | C | GLU | A | 200 | 98.939 | −39.293 | 105.581 | 1.00 | 122.92 | | C |
| ANISOU | 1427 | C | GLU | A | 200 | 16701 | 10016 | 19989 | −2453 | 8829 | −4530 | C |
| ATOM | 1428 | O | GLU | A | 200 | 99.075 | −39.318 | 106.806 | 1.00 | 122.03 | | O |
| ANISOU | 1428 | O | GLU | A | 200 | 16323 | 10292 | 19753 | −2853 | 8628 | −5120 | O |
| ATOM | 1429 | N | ALA | A | 201 | 97.981 | −39.973 | 104.913 | 1.00 | 119.43 | | N |
| ANISOU | 1429 | N | ALA | A | 201 | 16393 | 9699 | 19284 | −1795 | 8400 | −3723 | N |
| ATOM | 1430 | CA | ALA | A | 201 | 96.992 | −40.869 | 105.523 | 1.00 | 118.92 | | C |
| ANISOU | 1430 | CA | ALA | A | 201 | 16196 | 10195 | 18796 | −1500 | 7663 | −3440 | C |
| ATOM | 1431 | CB | ALA | A | 201 | 95.856 | −41.145 | 104.543 | 1.00 | 119.74 | | C |
| ANISOU | 1431 | CB | ALA | A | 201 | 16541 | 10205 | 18749 | −784 | 7569 | −2594 | C |
| ATOM | 1432 | C | ALA | A | 201 | 97.670 | −42.174 | 105.917 | 1.00 | 120.73 | | C |
| ANISOU | 1432 | C | ALA | A | 201 | 15977 | 11203 | 18691 | −1690 | 6795 | −3637 | C |
| ATOM | 1433 | O | ALA | A | 201 | 97.395 | −42.706 | 106.992 | 1.00 | 118.82 | | O |
| ANISOU | 1433 | O | ALA | A | 201 | 15518 | 11460 | 18166 | −1785 | 6324 | −3839 | O |
| ATOM | 1434 | N | PHE | A | 202 | 98.572 | −42.671 | 105.039 | 1.00 | 117.34 | | N |
| ANISOU | 1434 | N | PHE | A | 202 | 15428 | 10865 | 18293 | −1714 | 6631 | −3557 | N |
| ATOM | 1435 | CA | PHE | A | 202 | 99.341 | −43.898 | 105.233 | 1.00 | 117.00 | | C |
| ANISOU | 1435 | CA | PHE | A | 202 | 14989 | 11503 | 17963 | −1836 | 5897 | −3686 | C |
| ATOM | 1436 | CB | PHE | A | 202 | 100.178 | −44.242 | 103.975 | 1.00 | 118.43 | | C |
| ANISOU | 1436 | CB | PHE | A | 202 | 15149 | 11595 | 18256 | −1771 | 5886 | −3477 | C |
| ATOM | 1437 | CG | PHE | A | 202 | 101.020 | −45.486 | 104.117 | 1.00 | 119.23 | | C |
| ANISOU | 1437 | CG | PHE | A | 202 | 14862 | 12357 | 18083 | −1863 | 5196 | −3586 | C |
| ATOM | 1438 | CD1 | PHE | A | 202 | 100.437 | −46.743 | 104.108 | 1.00 | 121.85 | | C |
| ANISOU | 1438 | CD1 | PHE | A | 202 | 15117 | 13112 | 18069 | −1510 | 4546 | −3151 | C |
| ATOM | 1439 | CE1 | PHE | A | 202 | 101.212 | −47.891 | 104.257 | 1.00 | 122.49 | | C |
| ANISOU | 1439 | CE1 | PHE | A | 202 | 14882 | 13750 | 17909 | −1546 | 3993 | −3213 | C |
| ATOM | 1440 | CZ | PHE | A | 202 | 102.569 | −47.786 | 104.432 | 1.00 | 121.72 | | C |
| ANISOU | 1440 | CZ | PHE | A | 202 | 14497 | 13880 | 17870 | −1913 | 4026 | −3712 | C |
| ATOM | 1441 | CE2 | PHE | A | 202 | 103.170 | −46.548 | 104.426 | 1.00 | 123.60 | | C |
| ANISOU | 1441 | CE2 | PHE | A | 202 | 14760 | 13762 | 18442 | −2310 | 4633 | −4200 | C |
| ATOM | 1442 | CD2 | PHE | A | 202 | 102.398 | −45.398 | 104.264 | 1.00 | 120.79 | | C |
| ANISOU | 1442 | CD2 | PHE | A | 202 | 14767 | 12757 | 18372 | −2298 | 5249 | −4138 | C |
| ATOM | 1443 | C | PHE | A | 202 | 100.226 | −43.792 | 106.471 | 1.00 | 122.00 | | C |
| ANISOU | 1443 | C | PHE | A | 202 | 15261 | 12562 | 18530 | −2383 | 5809 | −4475 | C |
| ATOM | 1444 | O | PHE | A | 202 | 100.132 | −44.653 | 107.341 | 1.00 | 121.12 | | O |
| ANISOU | 1444 | O | PHE | A | 202 | 14886 | 13083 | 18049 | −2356 | 5211 | −4545 | O |
| ATOM | 1445 | N | ALA | A | 203 | 101.055 | −42.719 | 106.554 | 1.00 | 119.83 | | N |
| ANISOU | 1445 | N | ALA | A | 203 | 14969 | 11959 | 18600 | −2867 | 6453 | −5079 | N |
| ATOM | 1446 | CA | ALA | A | 203 | 101.993 | −42.432 | 107.646 | 1.00 | 119.79 | | C |
| ANISOU | 1446 | CA | ALA | A | 203 | 14565 | 12394 | 18556 | −3461 | 6488 | −5966 | C |
| ATOM | 1447 | CB | ALA | A | 203 | 102.864 | −41.233 | 107.300 | 1.00 | 120.49 | | C |
| ANISOU | 1447 | CB | ALA | A | 203 | 14708 | 11944 | 19128 | −3972 | 7340 | −6544 | C |
| ATOM | 1448 | C | ALA | A | 203 | 101.291 | −42.203 | 108.974 | 1.00 | 123.55 | | C |
| ANISOU | 1448 | C | ALA | A | 203 | 14983 | 13121 | 18840 | −3555 | 6421 | −6257 | C |
| ATOM | 1449 | O | ALA | A | 203 | 101.841 | −42.562 | 110.015 | 1.00 | 123.48 | | O |
| ANISOU | 1449 | O | ALA | A | 203 | 14541 | 13849 | 18527 | −3828 | 6050 | −6793 | O |
| ATOM | 1450 | N | SER | A | 204 | 100.085 | −41.614 | 108.939 | 1.00 | 119.67 | | N |
| ANISOU | 1450 | N | SER | A | 204 | 14906 | 12071 | 18490 | −3293 | 6778 | −5896 | N |
| ATOM | 1451 | CA | SER | A | 204 | 99.288 | −41.343 | 110.133 | 1.00 | 119.63 | | C |
| ANISOU | 1451 | CA | SER | A | 204 | 14905 | 12219 | 18329 | −3342 | 6765 | −6110 | C |
| ATOM | 1452 | CB | SER | A | 204 | 98.141 | −40.392 | 109.794 | 1.00 | 123.11 | | C |
| ANISOU | 1452 | CB | SER | A | 204 | 15861 | 11844 | 19069 | −3080 | 7413 | −5734 | C |
| ATOM | 1453 | OG | SER | A | 204 | 97.426 | −39.978 | 110.943 | 1.00 | 129.86 | | O |
| ANISOU | 1453 | OG | SER | A | 204 | 16738 | 12769 | 19834 | −3183 | 7510 | −6009 | O |
| ATOM | 1454 | C | SER | A | 204 | 98.753 | −42.652 | 110.718 | 1.00 | 123.18 | | C |
| ANISOU | 1454 | C | SER | A | 204 | 15155 | 13390 | 18255 | −2989 | 5880 | −5750 | C |
| ATOM | 1455 | O | SER | A | 204 | 98.910 | −42.911 | 111.914 | 1.00 | 123.06 | | O |
| ANISOU | 1455 | O | SER | A | 204 | 14830 | 13993 | 17934 | −3183 | 5569 | −6182 | O |
| ATOM | 1456 | N | LEU | A | 205 | 98.158 | −43.485 | 109.858 | 1.00 | 119.02 | | N |
| ANISOU | 1456 | N | LEU | A | 205 | 14795 | 12805 | 17621 | −2475 | 5516 | −4984 | N |
| ATOM | 1457 | CA | LEU | A | 205 | 97.567 | −44.759 | 110.234 | 1.00 | 118.60 | | C |
| ANISOU | 1457 | CA | LEU | A | 205 | 14625 | 13296 | 17141 | −2121 | 4791 | −4577 | C |
| ATOM | 1458 | CB | LEU | A | 205 | 96.636 | −45.244 | 109.104 | 1.00 | 118.11 | | C |
| ANISOU | 1458 | CB | LEU | A | 205 | 14848 | 12926 | 17102 | −1604 | 4682 | −3787 | C |
| ATOM | 1459 | CG | LEU | A | 205 | 95.840 | −46.530 | 109.297 | 1.00 | 121.72 | | C |
| ANISOU | 1459 | CG | LEU | A | 205 | 15250 | 13801 | 17198 | −1237 | 4064 | −3329 | C |
| ATOM | 1460 | CD1 | LEU | A | 205 | 95.023 | −46.521 | 110.583 | 1.00 | 121.82 | | C |
| ANISOU | 1460 | CD1 | LEU | A | 205 | 15246 | 14046 | 16995 | −1234 | 3950 | −3456 | C |
| ATOM | 1461 | CD2 | LEU | A | 205 | 94.960 | −46.790 | 108.100 | 1.00 | 122.33 | | C |
| ANISOU | 1461 | CD2 | LEU | A | 205 | 15562 | 13586 | 17333 | −810 | 4076 | −2691 | C |
| ATOM | 1462 | C | LEU | A | 205 | 98.622 | −45.797 | 110.604 | 1.00 | 124.00 | | C |
| ANISOU | 1462 | C | LEU | A | 205 | 14888 | 14718 | 17507 | −2224 | 4225 | −4789 | C |
| ATOM | 1463 | O | LEU | A | 205 | 98.448 | −46.489 | 111.606 | 1.00 | 123.71 | | O |
| ANISOU | 1463 | O | LEU | A | 205 | 14652 | 15259 | 17094 | −2146 | 3791 | −4843 | O |
| ATOM | 1464 | N | SER | A | 206 | 99.719 | −45.886 | 109.819 | 1.00 | 121.83 | | N |
| ANISOU | 1464 | N | SER | A | 206 | 14482 | 14438 | 17370 | −2368 | 4264 | −4898 | N |
| ATOM | 1465 | CA | SER | A | 206 | 100.804 | −46.845 | 110.059 | 1.00 | 122.26 | | C |
| ANISOU | 1465 | CA | SER | A | 206 | 14127 | 15198 | 17128 | −2418 | 3770 | −5073 | C |
| ATOM | 1466 | C | SER | A | 206 | 101.506 | −46.640 | 111.405 | 1.00 | 127.31 | | C |
| ANISOU | 1466 | C | SER | A | 206 | 14345 | 16523 | 17506 | −2769 | 3682 | −5807 | C |
| ATOM | 1467 | O | SER | A | 206 | 101.703 | −47.617 | 112.133 | 1.00 | 126.80 | | O |
| ANISOU | 1467 | O | SER | A | 206 | 14003 | 17182 | 16992 | −2576 | 3154 | −5761 | O |
| ATOM | 1468 | CB | SER | A | 206 | 101.805 | −46.877 | 108.905 | 1.00 | 126.03 | | C |
| ANISOU | 1468 | CB | SER | A | 206 | 14557 | 15493 | 17834 | −2510 | 3885 | −5057 | C |
| ATOM | 1469 | OG | SER | A | 206 | 102.347 | −45.609 | 108.579 | 1.00 | 135.99 | | O |
| ANISOU | 1469 | OG | SER | A | 206 | 15879 | 16284 | 19506 | −2930 | 4546 | −5529 | O |
| ATOM | 1470 | N | ALA | A | 207 | 101.845 | −45.376 | 111.751 | 1.00 | 124.81 | | N |
| ANISOU | 1470 | N | ALA | A | 207 | 13978 | 15993 | 17450 | −3261 | 4240 | −6486 | N |
| ATOM | 1471 | CA | ALA | A | 207 | 102.483 | −45.037 | 113.028 | 1.00 | 124.99 | | C |
| ANISOU | 1471 | CA | ALA | A | 207 | 13555 | 16712 | 17225 | −3661 | 4225 | −7307 | C |
| ATOM | 1472 | CB | ALA | A | 207 | 102.909 | −43.577 | 113.042 | 1.00 | 125.70 | | C |
| ANISOU | 1472 | CB | ALA | A | 207 | 13652 | 16357 | 17751 | −4274 | 5001 | −8063 | C |
| ATOM | 1473 | C | ALA | A | 207 | 101.540 | −45.337 | 114.208 | 1.00 | 129.54 | | C |
| ANISOU | 1473 | C | ALA | A | 207 | 14134 | 17656 | 17428 | −3444 | 3935 | −7211 | C |
| ATOM | 1474 | O | ALA | A | 207 | 102.018 | −45.744 | 115.272 | 1.00 | 129.68 | | O |
| ANISOU | 1474 | O | ALA | A | 207 | 13721 | 18565 | 16989 | −3490 | 3582 | −7597 | O |
| ATOM | 1475 | N | ARG | A | 208 | 100.206 | −45.176 | 114.003 | 1.00 | 125.70 | | N |
| ANISOU | 1475 | N | ARG | A | 208 | 14115 | 16544 | 17101 | −3165 | 4075 | −6674 | N |
| ATOM | 1476 | CA | ARG | A | 208 | 99.181 | −45.470 | 115.012 | 1.00 | 125.53 | | C |
| ANISOU | 1476 | CA | ARG | A | 208 | 14157 | 16767 | 16772 | −2931 | 3837 | −6504 | C |
| ATOM | 1477 | C | ARG | A | 208 | 99.181 | −46.975 | 115.350 | 1.00 | 131.16 | | C |
| ANISOU | 1477 | C | ARG | A | 208 | 14692 | 18161 | 16984 | −2486 | 3131 | −6053 | C |
| ATOM | 1478 | O | ARG | A | 208 | 99.180 | −47.331 | 116.528 | 1.00 | 130.93 | | O |
| ANISOU | 1478 | O | ARG | A | 208 | 14411 | 18814 | 16524 | −2431 | 2852 | −6261 | O |
| ATOM | 1479 | CB | ARG | A | 208 | 97.790 | −44.997 | 114.550 | 1.00 | 123.24 | | C |
| ANISOU | 1479 | CB | ARG | A | 208 | 14384 | 15665 | 16778 | −2702 | 4157 | −5999 | C |
| ATOM | 1480 | CG | ARG | A | 208 | 97.387 | −43.616 | 115.066 | 1.00 | 124.89 | | C |
| ANISOU | 1480 | CG | ARG | A | 208 | 14748 | 15440 | 17263 | −3044 | 4796 | −6483 | C |
| ATOM | 1481 | CD | ARG | A | 208 | 95.890 | −43.363 | 114.941 | 1.00 | 128.56 | | C |
| ANISOU | 1481 | CD | ARG | A | 208 | 15649 | 15351 | 17848 | −2698 | 4974 | −5943 | C |
| ATOM | 1482 | NE | ARG | A | 208 | 95.110 | −44.101 | 115.946 | 1.00 | 132.85 | | N |
| ANISOU | 1482 | NE | ARG | A | 208 | 16116 | 16391 | 17970 | −2451 | 4503 | −5762 | N |
| ATOM | 1483 | CZ | ARG | A | 208 | 93.779 | −44.092 | 116.029 | 1.00 | 145.48 | | C |
| ANISOU | 1483 | CZ | ARG | A | 208 | 18008 | 17697 | 19570 | −2135 | 4536 | −5316 | C |
| ATOM | 1484 | NH1 | ARG | A | 208 | 93.056 | −43.379 | 115.176 | 1.00 | 131.82 | | N |
| ANISOU | 1484 | NH1 | ARG | A | 208 | 16649 | 15236 | 18201 | −1977 | 4996 | −4984 | N |
| ATOM | 1485 | NH2 | ARG | A | 208 | 93.166 | −44.792 | 116.974 | 1.00 | 134.42 | | N |
| ANISOU | 1485 | NH2 | ARG | A | 208 | 16520 | 16764 | 17790 | −1947 | 4133 | −5194 | N |
| ATOM | 1486 | N | ILE | A | 209 | 99.241 | −47.845 | 114.317 | 1.00 | 128.61 | | N |
| ANISOU | 1486 | N | ILE | A | 209 | 14493 | 17662 | 16711 | −2170 | 2893 | −5460 | N |
| ATOM | 1487 | CA | ILE | A | 209 | 99.303 | −49.306 | 114.452 | 1.00 | 128.80 | | C |
| ANISOU | 1487 | CA | ILE | A | 209 | 14405 | 18191 | 16341 | −1747 | 2340 | −4995 | C |
| ATOM | 1488 | C | ILE | A | 209 | 100.712 | −49.661 | 114.947 | 1.00 | 135.77 | | C |
| ANISOU | 1488 | C | ILE | A | 209 | 14788 | 19903 | 16897 | −1856 | 2096 | −5436 | C |
| ATOM | 1489 | O | ILE | A | 209 | 101.667 | −49.720 | 114.154 | 1.00 | 136.43 | | O |
| ANISOU | 1489 | O | ILE | A | 209 | 14735 | 19978 | 17123 | −1967 | 2113 | −5522 | O |
| ATOM | 1490 | CB | ILE | A | 209 | 98.904 | −49.989 | 113.115 | 1.00 | 131.36 | | C |
| ANISOU | 1490 | CB | ILE | A | 209 | 15026 | 18003 | 16881 | −1448 | 2266 | −4312 | C |
| ATOM | 1491 | CG1 | ILE | A | 209 | 97.460 | −49.603 | 112.759 | 1.00 | 131.49 | | C |
| ANISOU | 1491 | CG1 | ILE | A | 209 | 15451 | 17382 | 17127 | −1309 | 2491 | −3937 | C |
| ATOM | 1492 | CG2 | ILE | A | 209 | 99.092 | −51.521 | 113.163 | 1.00 | 131.75 | | C |
| ANISOU | 1492 | CG2 | ILE | A | 209 | 14977 | 18496 | 16586 | −1054 | 1797 | −3872 | C |
| ATOM | 1493 | CD1 | ILE | A | 209 | 97.139 | −49.627 | 111.393 | 1.00 | 137.81 | | C |
| ANISOU | 1493 | CD1 | ILE | A | 209 | 16499 | 17639 | 18222 | −1171 | 2623 | −3518 | C |
| ATOM | 1494 | N | GLN | A | 210 | 100.837 | −49.816 | 116.283 | 1.00 | 133.13 | | N |
| ANISOU | 1494 | N | GLN | A | 210 | 14152 | 20329 | 16101 | −1822 | 1894 | −5752 | N |
| ATOM | 1495 | CA | GLN | A | 210 | 102.085 | −50.091 | 117.011 | 1.00 | 133.34 | | C |
| ANISOU | 1495 | CA | GLN | A | 210 | 13618 | 21362 | 15684 | −1869 | 1648 | −6235 | C |
| ATOM | 1496 | CB | GLN | A | 210 | 101.790 | −50.347 | 118.501 | 1.00 | 134.66 | | C |
| ANISOU | 1496 | CB | GLN | A | 210 | 13565 | 22307 | 15293 | −1677 | 1425 | −6388 | C |
| ATOM | 1497 | C | GLN | A | 210 | 102.953 | −51.235 | 116.414 | 1.00 | 137.58 | | C |
| ANISOU | 1497 | C | GLN | A | 210 | 13993 | 22241 | 16039 | −1531 | 1312 | −5860 | C |
| ATOM | 1498 | O | GLN | A | 210 | 104.186 | −51.103 | 116.361 | 1.00 | 137.42 | | O |
| ANISOU | 1498 | O | GLN | A | 210 | 13548 | 22753 | 15914 | −1723 | 1273 | −6324 | O |
| ATOM | 1499 | N | ALA | A | 211 | 102.298 | −52.326 | 115.936 | 1.00 | 133.03 | | N |
| ANISOU | 1499 | N | ALA | A | 211 | 13753 | 21338 | 15453 | −1058 | 1118 | −5059 | N |
| ATOM | 1500 | CA | ALA | A | 211 | 102.944 | −53.510 | 115.356 | 1.00 | 131.85 | | C |
| ANISOU | 1500 | CA | ALA | A | 211 | 13542 | 21394 | 15163 | −688 | 855 | −4613 | C |
| ATOM | 1501 | CB | ALA | A | 211 | 102.258 | −54.774 | 115.857 | 1.00 | 132.48 | | C |
| ANISOU | 1501 | CB | ALA | A | 211 | 13805 | 21627 | 14903 | −121 | 625 | −3967 | C |
| ATOM | 1502 | C | ALA | A | 211 | 103.023 | −53.518 | 113.819 | 1.00 | 133.23 | | C |
| ANISOU | 1502 | C | ALA | A | 211 | 13978 | 20811 | 15834 | −789 | 998 | −4333 | C |
| ATOM | 1503 | O | ALA | A | 211 | 102.281 | −52.796 | 113.147 | 1.00 | 132.91 | | O |
| ANISOU | 1503 | O | ALA | A | 211 | 14266 | 20007 | 16226 | −1007 | 1282 | −4280 | O |
| ATOM | 1504 | N | THR | A | 212 | 103.932 | −54.357 | 113.278 | 1.00 | 127.57 | | N |
| ANISOU | 1504 | N | THR | A | 212 | 13103 | 20353 | 15016 | −580 | 811 | −4133 | N |
| ATOM | 1505 | CA | THR | A | 212 | 104.170 | −54.558 | 111.842 | 1.00 | 126.36 | | C |
| ANISOU | 1505 | CA | THR | A | 212 | 13140 | 19623 | 15249 | −615 | 895 | −3853 | C |
| ATOM | 1506 | CB | THR | A | 212 | 105.679 | −54.785 | 111.589 | 1.00 | 134.09 | | C |
| ANISOU | 1506 | CB | THR | A | 212 | 13704 | 21144 | 16098 | −651 | 775 | −4119 | C |
| ATOM | 1507 | OG1 | THR | A | 212 | 106.096 | −55.998 | 112.225 | 1.00 | 132.73 | | O |
| ANISOU | 1507 | OG1 | THR | A | 212 | 13334 | 21671 | 15426 | −152 | 460 | −3827 | O |
| ATOM | 1508 | CG2 | THR | A | 212 | 106.550 | −53.607 | 112.042 | 1.00 | 132.45 | | C |
| ANISOU | 1508 | CG2 | THR | A | 212 | 13082 | 21347 | 15895 | −1145 | 936 | −4961 | C |
| ATOM | 1509 | C | THR | A | 212 | 103.346 | −55.772 | 111.349 | 1.00 | 127.71 | | C |
| ANISOU | 1509 | C | THR | A | 212 | 13670 | 19428 | 15427 | −188 | 777 | −3105 | C |
| ATOM | 1510 | O | THR | A | 212 | 102.792 | −56.494 | 112.182 | 1.00 | 127.57 | | O |
| ANISOU | 1510 | O | THR | A | 212 | 13712 | 19660 | 15098 | 138 | 644 | −2835 | O |
| ATOM | 1511 | N | GLN | A | 213 | 103.312 | −56.033 | 110.008 | 1.00 | 122.01 | | N |
| ANISOU | 1511 | N | GLN | A | 213 | 13165 | 18151 | 15043 | −192 | 855 | −2801 | N |
| ATOM | 1512 | CA | GLN | A | 213 | 102.594 | −57.181 | 109.418 | 1.00 | 120.40 | | C |
| ANISOU | 1512 | CA | GLN | A | 213 | 13259 | 17607 | 14878 | 134 | 794 | −2193 | C |
| ATOM | 1513 | C | GLN | A | 213 | 103.175 | −58.528 | 109.868 | 1.00 | 123.90 | | C |
| ANISOU | 1513 | C | GLN | A | 213 | 13577 | 18539 | 14959 | 552 | 590 | −1904 | C |
| ATOM | 1514 | O | GLN | A | 213 | 102.448 | −59.526 | 109.879 | 1.00 | 123.08 | | O |
| ANISOU | 1514 | O | GLN | A | 213 | 13716 | 18247 | 14801 | 839 | 595 | −1455 | O |
| ATOM | 1515 | CB | GLN | A | 213 | 102.512 | −57.095 | 107.887 | 1.00 | 120.78 | | C |
| ANISOU | 1515 | CB | GLN | A | 213 | 13503 | 17062 | 15324 | 20 | 928 | −2012 | C |
| ATOM | 1516 | CG | GLN | A | 213 | 101.150 | −57.539 | 107.324 | 1.00 | 109.09 | | C |
| ANISOU | 1516 | CG | GLN | A | 213 | 12371 | 15086 | 13990 | 148 | 1001 | −1596 | C |
| ATOM | 1517 | CD | GLN | A | 213 | 100.067 | −56.461 | 107.279 | 1.00 | 107.84 | | C |
| ANISOU | 1517 | CD | GLN | A | 213 | 12398 | 14550 | 14026 | −25 | 1200 | −1686 | C |
| ATOM | 1518 | OE1 | GLN | A | 213 | 100.226 | −55.324 | 107.756 | 1.00 | 96.54 | | O |
| ANISOU | 1518 | OE1 | GLN | A | 213 | 10891 | 13139 | 12649 | −256 | 1336 | −2056 | O |
| ATOM | 1519 | NE2 | GLN | A | 213 | 98.922 | −56.799 | 106.694 | 1.00 | 94.51 | | N |
| ANISOU | 1519 | NE2 | GLN | A | 213 | 10944 | 12526 | 12441 | 88 | 1256 | −1368 | N |
| ATOM | 1520 | N | GLU | A | 214 | 104.469 | −58.542 | 110.279 | 1.00 | 120.72 | | N |
| ANISOU | 1520 | N | GLU | A | 214 | 12785 | 18787 | 14296 | 597 | 456 | −2177 | N |
| ATOM | 1521 | CA | GLU | A | 214 | 105.162 | −59.720 | 110.817 | 1.00 | 120.56 | | C |
| ANISOU | 1521 | CA | GLU | A | 214 | 12592 | 19358 | 13856 | 1076 | 289 | −1911 | C |
| ATOM | 1522 | CB | GLU | A | 214 | 106.660 | −59.420 | 111.025 | 1.00 | 121.94 | | C |
| ANISOU | 1522 | CB | GLU | A | 214 | 12259 | 20275 | 13798 | 1029 | 154 | −2337 | C |
| ATOM | 1523 | C | GLU | A | 214 | 104.496 | −60.131 | 112.146 | 1.00 | 124.25 | | C |
| ANISOU | 1523 | C | GLU | A | 214 | 13104 | 20177 | 13928 | 1402 | 243 | −1744 | C |
| ATOM | 1524 | O | GLU | A | 214 | 104.082 | −61.286 | 112.294 | 1.00 | 124.30 | | O |
| ANISOU | 1524 | O | GLU | A | 214 | 13339 | 20086 | 13804 | 1821 | 285 | −1223 | O |
| ATOM | 1525 | N | ALA | A | 215 | 104.338 | −59.155 | 113.076 | 1.00 | 119.66 | | N |
| ANISOU | 1525 | N | ALA | A | 215 | 12333 | 19939 | 13192 | 1181 | 217 | −2198 | N |
| ATOM | 1526 | CA | ALA | A | 215 | 103.700 | −59.325 | 114.387 | 1.00 | 118.58 | | C |
| ANISOU | 1526 | CA | ALA | A | 215 | 12210 | 20174 | 12672 | 1433 | 178 | −2128 | C |
| ATOM | 1527 | CB | ALA | A | 215 | 103.912 | −58.085 | 115.250 | 1.00 | 119.30 | | C |
| ANISOU | 1527 | CB | ALA | A | 215 | 11968 | 20754 | 12608 | 1086 | 147 | −2807 | C |
| ATOM | 1528 | C | ALA | A | 215 | 102.206 | −59.637 | 114.269 | 1.00 | 119.46 | | C |
| ANISOU | 1528 | C | ALA | A | 215 | 12807 | 19567 | 13017 | 1471 | 336 | −1722 | C |
| ATOM | 1529 | O | ALA | A | 215 | 101.702 | −60.422 | 115.072 | 1.00 | 119.70 | | O |
| ANISOU | 1529 | O | ALA | A | 215 | 12963 | 19764 | 12753 | 1862 | 350 | −1377 | O |
| ATOM | 1530 | N | ARG | A | 216 | 101.503 | −59.030 | 113.279 | 1.00 | 112.53 | | N |
| ANISOU | 1530 | N | ARG | A | 216 | 12183 | 17932 | 12640 | 1095 | 479 | −1762 | N |
| ATOM | 1531 | CA | ARG | A | 216 | 100.065 | −59.216 | 113.025 | 1.00 | 110.49 | | C |
| ANISOU | 1531 | CA | ARG | A | 216 | 12322 | 17040 | 12619 | 1082 | 627 | −1453 | C |
| ATOM | 1532 | CB | ARG | A | 216 | 99.585 | −58.287 | 111.895 | 1.00 | 108.00 | | C |
| ANISOU | 1532 | CB | ARG | A | 216 | 12158 | 16095 | 12784 | 688 | 766 | −1597 | C |
| ATOM | 1533 | CG | ARG | A | 216 | 98.922 | −57.005 | 112.361 | 1.00 | 110.28 | | C |
| ANISOU | 1533 | CG | ARG | A | 216 | 12473 | 16257 | 13173 | 399 | 882 | −1942 | C |
| ATOM | 1534 | CD | ARG | A | 216 | 98.654 | −56.086 | 111.186 | 1.00 | 109.83 | | C |
| ANISOU | 1534 | CD | ARG | A | 216 | 12554 | 15622 | 13556 | 105 | 1074 | −2035 | C |
| ATOM | 1535 | NE | ARG | A | 216 | 97.915 | −54.890 | 111.588 | 1.00 | 115.21 | | N |
| ANISOU | 1535 | NE | ARG | A | 216 | 13320 | 16093 | 14360 | −116 | 1267 | −2295 | N |
| ATOM | 1536 | CZ | ARG | A | 216 | 96.588 | −54.803 | 111.606 | 1.00 | 131.83 | | C |
| ANISOU | 1536 | CZ | ARG | A | 216 | 15672 | 17870 | 16549 | −48 | 1367 | −2080 | C |
| ATOM | 1537 | NH1 | ARG | A | 216 | 95.842 | −55.836 | 111.234 | 1.00 | 119.83 | | N |
| ANISOU | 1537 | NH1 | ARG | A | 216 | 14312 | 16207 | 15010 | 183 | 1299 | −1655 | N |
| ATOM | 1538 | NH2 | ARG | A | 216 | 95.997 | −53.680 | 111.993 | 1.00 | 119.11 | | N |
| ANISOU | 1538 | NH2 | ARG | A | 216 | 14133 | 16078 | 15045 | −224 | 1572 | −2318 | N |
| ATOM | 1539 | C | ARG | A | 216 | 99.726 | −60.676 | 112.688 | 1.00 | 111.46 | | C |
| ANISOU | 1539 | C | ARG | A | 216 | 12688 | 16934 | 12728 | 1440 | 692 | −894 | C |
| ATOM | 1540 | O | ARG | A | 216 | 98.878 | −61.273 | 113.361 | 1.00 | 110.09 | | O |
| ANISOU | 1540 | O | ARG | A | 216 | 12704 | 16712 | 12414 | 1660 | 781 | −631 | O |
| ATOM | 1541 | N | GLU | A | 217 | 100.420 | −61.248 | 111.667 | 1.00 | 106.35 | | N |
| ANISOU | 1541 | N | GLU | A | 217 | 12035 | 16139 | 12235 | 1485 | 691 | −740 | N |
| ATOM | 1542 | CA | GLU | A | 217 | 100.237 | −62.624 | 111.176 | 1.00 | 105.08 | | C |
| ANISOU | 1542 | CA | GLU | A | 217 | 12101 | 15700 | 12126 | 1766 | 824 | −272 | C |
| ATOM | 1543 | CB | GLU | A | 217 | 101.009 | −62.834 | 109.863 | 1.00 | 106.22 | | C |
| ANISOU | 1543 | CB | GLU | A | 217 | 12202 | 15634 | 12522 | 1665 | 815 | −252 | C |
| ATOM | 1544 | C | GLU | A | 217 | 100.601 | −63.689 | 112.218 | 1.00 | 106.99 | | C |
| ANISOU | 1544 | C | GLU | A | 217 | 12330 | 16378 | 11944 | 2287 | 862 | 46 | C |
| ATOM | 1545 | O | GLU | A | 217 | 100.035 | −64.794 | 112.210 | 1.00 | 105.65 | | O |
| ANISOU | 1545 | O | GLU | A | 217 | 12441 | 15902 | 11800 | 2532 | 1096 | 447 | O |
| ATOM | 1546 | N | ARG | A | 218 | 101.537 | −63.340 | 113.123 | 1.00 | 103.03 | | N |
| ANISOU | 1546 | N | ARG | A | 218 | 11492 | 16617 | 11037 | 2465 | 675 | −152 | N |
| ATOM | 1547 | CA | ARG | A | 218 | 101.987 | −64.201 | 114.215 | 1.00 | 102.53 | | C |
| ANISOU | 1547 | CA | ARG | A | 218 | 11347 | 17147 | 10461 | 3050 | 692 | 142 | C |
| ATOM | 1548 | CB | ARG | A | 218 | 103.244 | −63.620 | 114.891 | 1.00 | 104.02 | | C |
| ANISOU | 1548 | CB | ARG | A | 218 | 11023 | 18282 | 10217 | 3154 | 422 | −225 | C |
| ATOM | 1549 | C | ARG | A | 218 | 100.847 | −64.406 | 115.227 | 1.00 | 104.02 | | C |
| ANISOU | 1549 | C | ARG | A | 218 | 11768 | 17264 | 10488 | 3213 | 838 | 330 | C |
| ATOM | 1550 | O | ARG | A | 218 | 100.600 | −65.547 | 115.621 | 1.00 | 104.32 | | O |
| ANISOU | 1550 | O | ARG | A | 218 | 12041 | 17232 | 10365 | 3672 | 1077 | 815 | O |
| ATOM | 1551 | N | LEU | A | 219 | 100.130 | −63.302 | 115.598 | 1.00 | 97.14 | | N |
| ANISOU | 1551 | N | LEU | A | 219 | 10862 | 16348 | 9698 | 2831 | 751 | −45 | N |
| ATOM | 1552 | CA | LEU | A | 219 | 98.992 | −63.268 | 116.521 | 1.00 | 94.81 | | C |
| ANISOU | 1552 | CA | LEU | A | 219 | 10761 | 15975 | 9289 | 2892 | 866 | 43 | C |
| ATOM | 1553 | C | LEU | A | 219 | 97.827 | −64.024 | 115.933 | 1.00 | 96.94 | | C |
| ANISOU | 1553 | C | LEU | A | 219 | 11458 | 15458 | 9917 | 2846 | 1159 | 396 | C |
| ATOM | 1554 | O | LEU | A | 219 | 97.251 | −64.855 | 116.628 | 1.00 | 96.39 | | O |
| ANISOU | 1554 | O | LEU | A | 219 | 11611 | 15336 | 9678 | 3178 | 1387 | 748 | O |
| ATOM | 1555 | CB | LEU | A | 219 | 98.583 | −61.826 | 116.822 | 1.00 | 94.40 | | C |
| ANISOU | 1555 | CB | LEU | A | 219 | 10565 | 15982 | 9321 | 2435 | 732 | −484 | C |
| ATOM | 1556 | N | ILE | A | 220 | 97.502 | −63.774 | 114.644 | 1.00 | 93.21 | | N |
| ANISOU | 1556 | N | ILE | A | 220 | 11086 | 14411 | 9921 | 2450 | 1186 | 293 | N |
| ATOM | 1557 | CA | ILE | A | 220 | 96.414 | −64.450 | 113.921 | 1.00 | 93.32 | | C |
| ANISOU | 1557 | CA | ILE | A | 220 | 11424 | 13752 | 10281 | 2333 | 1454 | 520 | C |
| ATOM | 1558 | C | ILE | A | 220 | 96.630 | −65.972 | 113.919 | 1.00 | 98.36 | | C |
| ANISOU | 1558 | C | ILE | A | 220 | 12267 | 14253 | 10853 | 2730 | 1749 | 976 | C |
| ATOM | 1559 | O | ILE | A | 220 | 95.685 | −66.721 | 114.191 | 1.00 | 98.39 | | O |
| ANISOU | 1559 | O | ILE | A | 220 | 12546 | 13920 | 10920 | 2813 | 2069 | 1207 | O |
| ATOM | 1560 | CB | ILE | A | 220 | 96.182 | −63.845 | 112.502 | 1.00 | 96.23 | | C |
| ANISOU | 1560 | CB | ILE | A | 220 | 11782 | 13697 | 11086 | 1894 | 1396 | 300 | C |
| ATOM | 1561 | CG1 | ILE | A | 220 | 95.714 | −62.381 | 112.596 | 1.00 | 96.85 | | C |
| ANISOU | 1561 | CG1 | ILE | A | 220 | 11754 | 13793 | 11251 | 1562 | 1248 | −72 | C |
| ATOM | 1562 | CG2 | ILE | A | 220 | 95.205 | −64.661 | 111.659 | 1.00 | 96.16 | | C |
| ANISOU | 1562 | CG2 | ILE | A | 220 | 12019 | 13138 | 11380 | 1783 | 1662 | 478 | C |
| ATOM | 1563 | N | GLN | A | 221 | 97.881 | −66.420 | 113.704 | 1.00 | 95.43 | | N |
| ANISOU | 1563 | N | GLN | A | 221 | 11762 | 14157 | 10339 | 2997 | 1687 | 1101 | N |
| ATOM | 1564 | CA | GLN | A | 221 | 98.195 | −67.849 | 113.733 | 1.00 | 95.46 | | C |
| ANISOU | 1564 | CA | GLN | A | 221 | 11976 | 14028 | 10267 | 3442 | 2026 | 1568 | C |
| ATOM | 1565 | C | GLN | A | 221 | 98.120 | −68.450 | 115.148 | 1.00 | 102.12 | | C |
| ANISOU | 1565 | C | GLN | A | 221 | 12921 | 15218 | 10663 | 3999 | 2221 | 1913 | C |
| ATOM | 1566 | O | GLN | A | 221 | 97.787 | −69.633 | 115.286 | 1.00 | 102.01 | | O |
| ANISOU | 1566 | O | GLN | A | 221 | 13230 | 14851 | 10678 | 4309 | 2679 | 2332 | O |
| ATOM | 1567 | CB | GLN | A | 221 | 99.527 | −68.147 | 113.050 | 1.00 | 96.11 | | C |
| ANISOU | 1567 | CB | GLN | A | 221 | 11887 | 14303 | 10327 | 3598 | 1924 | 1623 | C |
| ATOM | 1568 | CG | GLN | A | 221 | 99.406 | −69.268 | 112.008 | 1.00 | 101.00 | | C |
| ANISOU | 1568 | CG | GLN | A | 221 | 12783 | 14294 | 11297 | 3584 | 2291 | 1875 | C |
| ATOM | 1569 | CD | GLN | A | 221 | 98.515 | −68.966 | 110.804 | 1.00 | 107.01 | | C |
| ANISOU | 1569 | CD | GLN | A | 221 | 13639 | 14469 | 12552 | 2995 | 2320 | 1607 | C |
| ATOM | 1570 | OE1 | GLN | A | 221 | 98.375 | −67.815 | 110.351 | 1.00 | 97.30 | | O |
| ANISOU | 1570 | OE1 | GLN | A | 221 | 12221 | 13300 | 11449 | 2601 | 2002 | 1241 | O |
| ATOM | 1571 | NE2 | GLN | A | 221 | 97.904 | −70.010 | 110.245 | 1.00 | 95.03 | | N |
| ANISOU | 1571 | NE2 | GLN | A | 221 | 12406 | 12393 | 11307 | 2941 | 2748 | 1776 | N |
| ATOM | 1572 | N | LYS | A | 222 | 98.376 | −67.627 | 116.195 | 1.00 | 100.07 | | N |
| ANISOU | 1572 | N | LYS | A | 222 | 12397 | 15629 | 9996 | 4114 | 1926 | 1722 | N |
| ATOM | 1573 | CA | LYS | A | 222 | 98.267 | −68.060 | 117.588 | 1.00 | 100.37 | | C |
| ANISOU | 1573 | CA | LYS | A | 222 | 12491 | 16101 | 9544 | 4658 | 2072 | 2021 | C |
| ATOM | 1574 | C | LYS | A | 222 | 96.792 | −68.303 | 117.881 | 1.00 | 106.86 | | C |
| ANISOU | 1574 | C | LYS | A | 222 | 13673 | 16349 | 10579 | 4500 | 2396 | 2130 | C |
| ATOM | 1575 | O | LYS | A | 222 | 96.449 | −69.339 | 118.448 | 1.00 | 106.93 | | O |
| ANISOU | 1575 | O | LYS | A | 222 | 13981 | 16187 | 10460 | 4930 | 2830 | 2584 | O |
| ATOM | 1576 | CB | LYS | A | 222 | 98.853 | −67.015 | 118.550 | 1.00 | 102.39 | | C |
| ANISOU | 1576 | CB | LYS | A | 222 | 12317 | 17262 | 9323 | 4720 | 1656 | 1668 | C |
| ATOM | 1577 | N | ALA | A | 223 | 95.921 | −67.377 | 117.423 | 1.00 | 104.95 | | N |
| ANISOU | 1577 | N | ALA | A | 223 | 13413 | 15778 | 10684 | 3894 | 2237 | 1726 | N |
| ATOM | 1578 | CA | ALA | A | 223 | 94.470 | −67.435 | 117.572 | 1.00 | 105.71 | | C |
| ANISOU | 1578 | CA | ALA | A | 223 | 13777 | 15373 | 11015 | 3653 | 2489 | 1727 | C |
| ATOM | 1579 | C | ALA | A | 223 | 93.887 | −68.639 | 116.833 | 1.00 | 112.10 | | C |
| ANISOU | 1579 | C | ALA | A | 223 | 14929 | 15472 | 12191 | 3613 | 2978 | 1998 | C |
| ATOM | 1580 | O | ALA | A | 223 | 93.023 | −69.322 | 117.384 | 1.00 | 111.92 | | O |
| ANISOU | 1580 | O | ALA | A | 223 | 15187 | 15156 | 12180 | 3737 | 3391 | 2224 | O |
| ATOM | 1581 | CB | ALA | A | 223 | 93.846 | −66.148 | 117.059 | 1.00 | 106.39 | | C |
| ANISOU | 1581 | CB | ALA | A | 223 | 13717 | 15327 | 11378 | 3067 | 2199 | 1244 | C |
| ATOM | 1582 | N | ILE | A | 224 | 94.373 | −68.901 | 115.603 | 1.00 | 110.69 | | N |
| ANISOU | 1582 | N | ILE | A | 224 | 14721 | 15024 | 12311 | 3424 | 2968 | 1944 | N |
| ATOM | 1583 | CA | ILE | A | 224 | 93.958 | −70.023 | 114.756 | 1.00 | 112.11 | | C |
| ANISOU | 1583 | CA | ILE | A | 224 | 15175 | 14563 | 12860 | 3321 | 3436 | 2104 | C |
| ATOM | 1584 | C | ILE | A | 224 | 94.248 | −71.368 | 115.456 | 1.00 | 119.86 | | C |
| ANISOU | 1584 | C | ILE | A | 224 | 16455 | 15435 | 13652 | 3903 | 3972 | 2632 | C |
| ATOM | 1585 | O | ILE | A | 224 | 93.367 | −72.232 | 115.482 | 1.00 | 118.96 | | O |
| ANISOU | 1585 | O | ILE | A | 224 | 16659 | 14782 | 13761 | 3850 | 4523 | 2773 | O |
| ATOM | 1586 | CB | ILE | A | 224 | 94.585 | −69.897 | 113.328 | 1.00 | 115.46 | | C |
| ANISOU | 1586 | CB | ILE | A | 224 | 15451 | 14843 | 13575 | 3029 | 3251 | 1909 | C |
| ATOM | 1587 | CG1 | ILE | A | 224 | 93.862 | −68.813 | 112.503 | 1.00 | 115.82 | | C |
| ANISOU | 1587 | CG1 | ILE | A | 224 | 15328 | 14784 | 13896 | 2453 | 2949 | 1460 | C |
| ATOM | 1588 | CG2 | ILE | A | 224 | 94.615 | −71.241 | 112.564 | 1.00 | 116.46 | | C |
| ANISOU | 1588 | CG2 | ILE | A | 224 | 15832 | 14437 | 13982 | 3064 | 3762 | 2123 | C |
| ATOM | 1589 | CD1 | ILE | A | 224 | 94.608 | −68.360 | 111.298 | 1.00 | 122.36 | | C |
| ANISOU | 1589 | CD1 | ILE | A | 224 | 15957 | 15634 | 14900 | 2233 | 2671 | 1264 | C |
| ATOM | 1590 | N | HIS | A | 225 | 95.469 | −71.522 | 116.045 | 1.00 | 120.02 | | N |
| ANISOU | 1590 | N | HIS | A | 225 | 16360 | 15997 | 13247 | 4475 | 3846 | 2915 | N |
| ATOM | 1591 | CA | HIS | A | 225 | 95.911 | −72.723 | 116.786 | 1.00 | 121.36 | | C |
| ANISOU | 1591 | CA | HIS | A | 225 | 16789 | 16188 | 13132 | 5192 | 4348 | 3500 | C |
| ATOM | 1592 | C | HIS | A | 225 | 95.004 | −72.951 | 118.000 | 1.00 | 123.06 | | C |
| ANISOU | 1592 | C | HIS | A | 225 | 17241 | 16376 | 13141 | 5436 | 4676 | 3721 | C |
| ATOM | 1593 | O | HIS | A | 225 | 94.569 | −74.077 | 118.242 | 1.00 | 121.78 | | O |
| ANISOU | 1593 | O | HIS | A | 225 | 17481 | 15711 | 13078 | 5704 | 5364 | 4111 | O |
| ATOM | 1594 | CB | HIS | A | 225 | 97.392 | −72.591 | 117.235 | 1.00 | 123.55 | | C |
| ANISOU | 1594 | CB | HIS | A | 225 | 16771 | 17282 | 12889 | 5776 | 4023 | 3688 | C |
| ATOM | 1595 | CG | HIS | A | 225 | 97.952 | −73.793 | 117.961 | 1.00 | 128.16 | | C |
| ANISOU | 1595 | CG | HIS | A | 225 | 17594 | 17989 | 13113 | 6636 | 4533 | 4352 | C |
| ATOM | 1596 | ND1 | HIS | A | 225 | 98.867 | −74.649 | 117.352 | 1.00 | 130.53 | | N |
| ANISOU | 1596 | ND1 | HIS | A | 225 | 17973 | 18161 | 13462 | 6979 | 4787 | 4663 | N |
| ATOM | 1597 | CD2 | HIS | A | 225 | 97.738 | −74.225 | 119.231 | 1.00 | 130.47 | | C |
| ANISOU | 1597 | CD2 | HIS | A | 225 | 18057 | 18544 | 12970 | 7254 | 4844 | 4776 | C |
| ATOM | 1598 | CE1 | HIS | A | 225 | 99.165 | −75.571 | 118.259 | 1.00 | 130.08 | | C |
| ANISOU | 1598 | CE1 | HIS | A | 225 | 18145 | 18272 | 13008 | 7814 | 5268 | 5285 | C |
| ATOM | 1599 | NE2 | HIS | A | 225 | 98.506 | −75.366 | 119.402 | 1.00 | 130.31 | | N |
| ANISOU | 1599 | NE2 | HIS | A | 225 | 18242 | 18543 | 12727 | 8020 | 5327 | 5387 | N |
| ATOM | 1600 | N | LEU | A | 226 | 94.720 | −71.869 | 118.745 | 1.00 | 119.11 | | N |
| ANISOU | 1600 | N | LEU | A | 226 | 16498 | 16383 | 12374 | 5325 | 4227 | 3453 | N |
| ATOM | 1601 | CA | LEU | A | 226 | 93.869 | −71.859 | 119.930 | 1.00 | 118.74 | | C |
| ANISOU | 1601 | CA | LEU | A | 226 | 16609 | 16413 | 12093 | 5514 | 4429 | 3590 | C |
| ATOM | 1602 | C | LEU | A | 226 | 92.452 | −72.315 | 119.619 | 1.00 | 123.18 | | C |
| ANISOU | 1602 | C | LEU | A | 226 | 17511 | 16154 | 13137 | 5083 | 4918 | 3527 | C |
| ATOM | 1603 | O | LEU | A | 226 | 91.927 | −73.152 | 120.343 | 1.00 | 122.31 | | O |
| ANISOU | 1603 | O | LEU | A | 226 | 17742 | 15774 | 12956 | 5413 | 5497 | 3898 | O |
| ATOM | 1604 | CB | LEU | A | 226 | 93.856 | −70.455 | 120.568 | 1.00 | 118.53 | | C |
| ANISOU | 1604 | CB | LEU | A | 226 | 16207 | 17059 | 11768 | 5331 | 3805 | 3180 | C |
| ATOM | 1605 | CG | LEU | A | 226 | 94.755 | −70.189 | 121.797 | 1.00 | 122.79 | | C |
| ANISOU | 1605 | CG | LEU | A | 226 | 16489 | 18575 | 11591 | 5957 | 3551 | 3331 | C |
| ATOM | 1606 | CD2 | LEU | A | 226 | 96.227 | −70.505 | 121.543 | 1.00 | 125.49 | | C |
| ANISOU | 1606 | CD2 | LEU | A | 226 | 16601 | 19426 | 11653 | 6392 | 3395 | 3499 | C |
| ATOM | 1607 | CD1 | LEU | A | 226 | 94.235 | −70.870 | 123.054 | 1.00 | 122.73 | | C |
| ANISOU | 1607 | CD1 | LEU | A | 226 | 16761 | 18653 | 11217 | 6529 | 3995 | 3796 | C |
| ATOM | 1608 | N | LEU | A | 227 | 91.854 | −71.797 | 118.528 | 1.00 | 120.83 | | N |
| ANISOU | 1608 | N | LEU | A | 227 | 17107 | 15490 | 13312 | 4373 | 4725 | 3060 | N |
| ATOM | 1609 | CA | LEU | A | 227 | 90.506 | −72.167 | 118.105 | 1.00 | 121.24 | | C |
| ANISOU | 1609 | CA | LEU | A | 227 | 17377 | 14877 | 13811 | 3899 | 5140 | 2890 | C |
| ATOM | 1610 | C | LEU | A | 227 | 90.432 | −73.585 | 117.546 | 1.00 | 127.89 | | C |
| ANISOU | 1610 | C | LEU | A | 227 | 18561 | 15054 | 14976 | 3945 | 5868 | 3132 | C |
| ATOM | 1611 | O | LEU | A | 227 | 89.561 | −74.346 | 117.974 | 1.00 | 127.33 | | O |
| ANISOU | 1611 | O | LEU | A | 227 | 18806 | 14531 | 15042 | 3952 | 6497 | 3273 | O |
| ATOM | 1612 | CB | LEU | A | 227 | 89.935 | −71.160 | 117.096 | 1.00 | 120.99 | | C |
| ANISOU | 1612 | CB | LEU | A | 227 | 17075 | 14791 | 14103 | 3211 | 4703 | 2331 | C |
| ATOM | 1613 | CG | LEU | A | 227 | 88.720 | −70.362 | 117.553 | 1.00 | 125.03 | | C |
| ANISOU | 1613 | CG | LEU | A | 227 | 17525 | 15342 | 14640 | 2897 | 4577 | 2051 | C |
| ATOM | 1614 | CD1 | LEU | A | 227 | 88.195 | −69.528 | 116.430 | 1.00 | 124.74 | | C |
| ANISOU | 1614 | CD1 | LEU | A | 227 | 17248 | 15232 | 14916 | 2323 | 4247 | 1585 | C |
| ATOM | 1615 | CD2 | LEU | A | 227 | 87.606 | −71.269 | 118.062 | 1.00 | 126.91 | | C |
| ANISOU | 1615 | CD2 | LEU | A | 227 | 18081 | 15126 | 15013 | 2883 | 5231 | 2190 | C |
| ATOM | 1616 | N | GLN | A | 228 | 91.353 | −73.945 | 116.610 | 1.00 | 126.76 | | N |
| ANISOU | 1616 | N | GLN | A | 228 | 18364 | 14832 | 14966 | 3967 | 5831 | 3166 | N |
| ATOM | 1617 | CA | GLN | A | 228 | 91.438 | −75.271 | 115.983 | 1.00 | 127.42 | | C |
| ANISOU | 1617 | CA | GLN | A | 228 | 18760 | 14279 | 15374 | 4001 | 6534 | 3364 | C |
| ATOM | 1618 | C | GLN | A | 228 | 91.516 | −76.374 | 117.054 | 1.00 | 134.86 | | C |
| ANISOU | 1618 | C | GLN | A | 228 | 20125 | 15006 | 16111 | 4667 | 7270 | 3969 | C |
| ATOM | 1619 | O | GLN | A | 228 | 90.734 | −77.329 | 117.006 | 1.00 | 133.98 | | O |
| ANISOU | 1619 | O | GLN | A | 228 | 20368 | 14210 | 16329 | 4526 | 8054 | 4028 | O |
| ATOM | 1620 | CB | GLN | A | 228 | 92.641 | −75.335 | 115.019 | 1.00 | 128.34 | | C |
| ANISOU | 1620 | CB | GLN | A | 228 | 18716 | 14510 | 15538 | 4059 | 6278 | 3368 | C |
| ATOM | 1621 | CG | GLN | A | 228 | 92.623 | −76.504 | 114.031 | 1.00 | 135.47 | | C |
| ANISOU | 1621 | CG | GLN | A | 228 | 19868 | 14721 | 16884 | 3874 | 6920 | 3380 | C |
| ATOM | 1622 | CD | GLN | A | 228 | 93.280 | −76.152 | 112.707 | 1.00 | 144.28 | | C |
| ANISOU | 1622 | CD | GLN | A | 228 | 20709 | 15918 | 18194 | 3540 | 6496 | 3074 | C |
| ATOM | 1623 | OE1 | GLN | A | 228 | 92.612 | −75.945 | 111.686 | 1.00 | 135.22 | | O |
| ANISOU | 1623 | OE1 | GLN | A | 228 | 19432 | 14540 | 17403 | 2904 | 6430 | 2601 | O |
| ATOM | 1624 | NE2 | GLN | A | 228 | 94.606 | −76.076 | 112.687 | 1.00 | 133.66 | | N |
| ANISOU | 1624 | NE2 | GLN | A | 228 | 19242 | 14953 | 16588 | 3981 | 6204 | 3330 | N |
| ATOM | 1625 | N | GLU | A | 229 | 92.400 | −76.194 | 118.055 | 1.00 | 134.57 | | N |
| ANISOU | 1625 | N | GLU | A | 229 | 20029 | 15591 | 15510 | 5385 | 7042 | 4386 | N |
| ATOM | 1626 | CA | GLU | A | 229 | 92.590 | −77.165 | 119.135 | 1.00 | 135.90 | | C |
| ANISOU | 1626 | CA | GLU | A | 229 | 20574 | 15696 | 15368 | 6174 | 7706 | 5047 | C |
| ATOM | 1627 | C | GLU | A | 229 | 91.405 | −77.213 | 120.112 | 1.00 | 143.58 | | C |
| ANISOU | 1627 | C | GLU | A | 229 | 21776 | 16475 | 16302 | 6155 | 8074 | 5103 | C |
| ATOM | 1628 | O | GLU | A | 229 | 91.135 | −78.281 | 120.666 | 1.00 | 143.13 | | O |
| ANISOU | 1628 | O | GLU | A | 229 | 22172 | 15950 | 16261 | 6570 | 8928 | 5570 | O |
| ATOM | 1629 | CB | GLU | A | 229 | 93.937 | −76.958 | 119.859 | 1.00 | 137.41 | | C |
| ANISOU | 1629 | CB | GLU | A | 229 | 20554 | 16763 | 14892 | 6996 | 7321 | 5454 | C |
| ATOM | 1630 | CG | GLU | A | 229 | 95.149 | −77.182 | 118.946 | 1.00 | 150.03 | | C |
| ANISOU | 1630 | CG | GLU | A | 229 | 21997 | 18471 | 16535 | 7121 | 7144 | 5502 | C |
| ATOM | 1631 | CD | GLU | A | 229 | 96.232 | −78.187 | 119.319 | 1.00 | 170.37 | | C |
| ANISOU | 1631 | CD | GLU | A | 229 | 24764 | 21207 | 18762 | 8063 | 7597 | 6196 | C |
| ATOM | 1632 | OE1 | GLU | A | 229 | 95.939 | −79.165 | 120.048 | 1.00 | 172.19 | | O |
| ANISOU | 1632 | OE1 | GLU | A | 229 | 25403 | 21101 | 18919 | 8524 | 8303 | 6680 | O |
| ATOM | 1633 | OE2 | GLU | A | 229 | 97.379 | −78.007 | 118.850 | 1.00 | 155.80 | | O |
| ANISOU | 1633 | OE2 | GLU | A | 229 | 22627 | 19820 | 16750 | 8253 | 7192 | 6194 | O |
| ATOM | 1634 | N | THR | A | 230 | 90.687 | −76.076 | 120.293 | 1.00 | 143.22 | | N |
| ANISOU | 1634 | N | THR | A | 230 | 21445 | 16739 | 16232 | 5675 | 7492 | 4636 | N |
| ATOM | 1635 | CA | THR | A | 230 | 89.511 | −75.966 | 121.163 | 1.00 | 144.58 | | C |
| ANISOU | 1635 | CA | THR | A | 230 | 21776 | 16775 | 16381 | 5575 | 7746 | 4607 | C |
| ATOM | 1636 | C | THR | A | 230 | 88.299 | −76.665 | 120.542 | 1.00 | 152.66 | | C |
| ANISOU | 1636 | C | THR | A | 230 | 23072 | 16907 | 18023 | 4970 | 8431 | 4356 | C |
| ATOM | 1637 | O | THR | A | 230 | 87.546 | −77.334 | 121.254 | 1.00 | 153.01 | | O |
| ANISOU | 1637 | O | THR | A | 230 | 23472 | 16549 | 18117 | 5111 | 9133 | 4586 | O |
| ATOM | 1638 | CB | THR | A | 230 | 89.262 | −74.500 | 121.538 | 1.00 | 154.03 | | C |
| ANISOU | 1638 | CB | THR | A | 230 | 22563 | 18629 | 17333 | 5305 | 6905 | 4197 | C |
| ATOM | 1639 | OG1 | THR | A | 230 | 90.367 | −74.049 | 122.323 | 1.00 | 154.31 | | O |
| ANISOU | 1639 | OG1 | THR | A | 230 | 22377 | 19505 | 16750 | 5925 | 6450 | 4443 | O |
| ATOM | 1640 | CG2 | THR | A | 230 | 87.957 | −74.283 | 122.317 | 1.00 | 153.33 | | C |
| ANISOU | 1640 | CG2 | THR | A | 230 | 22600 | 18388 | 17270 | 5105 | 7115 | 4087 | C |
| ATOM | 1641 | N | LEU | A | 231 | 88.119 | −76.522 | 119.225 | 1.00 | 152.13 | | N |
| ANISOU | 1641 | N | LEU | A | 231 | 22822 | 16574 | 18409 | 4303 | 8258 | 3866 | N |
| ATOM | 1642 | CA | LEU | A | 231 | 87.018 | −77.146 | 118.496 | 1.00 | 153.72 | | C |
| ANISOU | 1642 | CA | LEU | A | 231 | 23170 | 16058 | 19179 | 3655 | 8855 | 3499 | C |
| ATOM | 1643 | C | LEU | A | 231 | 87.191 | −78.659 | 118.386 | 1.00 | 162.75 | | C |
| ANISOU | 1643 | C | LEU | A | 231 | 24784 | 16468 | 20584 | 3887 | 9893 | 3850 | C |
| ATOM | 1644 | O | LEU | A | 231 | 86.239 | −79.395 | 118.663 | 1.00 | 162.28 | | O |
| ANISOU | 1644 | O | LEU | A | 231 | 25033 | 15823 | 20804 | 3694 | 10698 | 3819 | O |
| ATOM | 1645 | CB | LEU | A | 231 | 86.865 | −76.512 | 117.114 | 1.00 | 153.72 | | C |
| ANISOU | 1645 | CB | LEU | A | 231 | 22782 | 16132 | 19493 | 2954 | 8325 | 2881 | C |
| ATOM | 1646 | CG | LEU | A | 231 | 86.417 | −75.063 | 117.122 | 1.00 | 158.23 | | C |
| ANISOU | 1646 | CG | LEU | A | 231 | 22945 | 17259 | 19916 | 2640 | 7487 | 2489 | C |
| ATOM | 1647 | CD1 | LEU | A | 231 | 86.897 | −74.342 | 115.888 | 1.00 | 158.37 | | C |
| ANISOU | 1647 | CD1 | LEU | A | 231 | 22596 | 17528 | 20051 | 2295 | 6854 | 2128 | C |
| ATOM | 1648 | CD2 | LEU | A | 231 | 84.917 | −74.957 | 117.275 | 1.00 | 160.58 | | C |
| ANISOU | 1648 | CD2 | LEU | A | 231 | 23233 | 17350 | 20430 | 2160 | 7734 | 2129 | C |
| ATOM | 1649 | N | ALA | A | 232 | 88.418 | −79.120 | 118.027 | 1.00 | 163.10 | | N |
| ANISOU | 1649 | N | ALA | A | 232 | 24896 | 16537 | 20537 | 4320 | 9925 | 4192 | N |
| ATOM | 1650 | CA | ALA | A | 232 | 88.765 | −80.540 | 117.905 | 1.00 | 164.62 | | C |
| ANISOU | 1650 | CA | ALA | A | 232 | 25558 | 16033 | 20955 | 4642 | 10932 | 4595 | C |
| ATOM | 1651 | C | ALA | A | 232 | 88.652 | −81.288 | 119.243 | 1.00 | 172.28 | | C |
| ANISOU | 1651 | C | ALA | A | 232 | 26731 | 17093 | 21636 | 5062 | 11123 | 5009 | C |
| ATOM | 1652 | O | ALA | A | 232 | 88.450 | −82.503 | 119.246 | 1.00 | 171.47 | | O |
| ANISOU | 1652 | O | ALA | A | 232 | 26702 | 16684 | 21767 | 4760 | 11329 | 4915 | O |
| ATOM | 1653 | CB | ALA | A | 232 | 90.163 | −80.692 | 117.329 | 1.00 | 165.47 | | C |
| ANISOU | 1653 | CB | ALA | A | 232 | 25579 | 16364 | 20929 | 5033 | 10670 | 4849 | C |
| ATOM | 1654 | N | ARG | A | 233 | 88.757 | −80.562 | 120.372 | 1.00 | 171.73 | | N |
| ANISOU | 1654 | N | ARG | A | 233 | 26644 | 17574 | 21031 | 5646 | 10858 | 5363 | N |
| ATOM | 1655 | CA | ARG | A | 233 | 88.641 | −81.140 | 121.709 | 1.00 | 172.76 | | C |
| ANISOU | 1655 | CA | ARG | A | 233 | 26744 | 18056 | 20839 | 5805 | 10603 | 5548 | C |
| ATOM | 1656 | C | ARG | A | 233 | 87.181 | −81.247 | 122.205 | 1.00 | 180.08 | | C |
| ANISOU | 1656 | C | ARG | A | 233 | 27488 | 18950 | 21983 | 4973 | 10330 | 4999 | C |
| ATOM | 1657 | O | ARG | A | 233 | 86.937 | −81.969 | 123.177 | 1.00 | 180.44 | | O |
| ANISOU | 1657 | O | ARG | A | 233 | 27548 | 19113 | 21897 | 4970 | 10256 | 5101 | O |
| ATOM | 1658 | CB | ARG | A | 233 | 89.539 | −80.404 | 122.716 | 1.00 | 174.28 | | C |
| ANISOU | 1658 | CB | ARG | A | 233 | 26711 | 19184 | 20322 | 6494 | 9928 | 5894 | C |
| ATOM | 1659 | CG | ARG | A | 233 | 90.952 | −80.965 | 122.730 | 1.00 | 186.52 | | C |
| ANISOU | 1659 | CG | ARG | A | 233 | 27701 | 21439 | 21729 | 6133 | 8668 | 5593 | C |
| ATOM | 1660 | CD | ARG | A | 233 | 91.895 | −80.213 | 123.648 | 1.00 | 197.95 | | C |
| ANISOU | 1660 | CD | ARG | A | 233 | 28551 | 23967 | 22696 | 5974 | 7328 | 5292 | C |
| ATOM | 1661 | NE | ARG | A | 233 | 93.126 | −80.974 | 123.884 | 1.00 | 208.77 | | N |
| ANISOU | 1661 | NE | ARG | A | 233 | 29507 | 25737 | 24080 | 5441 | 6327 | 4928 | N |
| ATOM | 1662 | CZ | ARG | A | 233 | 94.209 | −80.923 | 123.112 | 1.00 | 222.20 | | C |
| ANISOU | 1662 | CZ | ARG | A | 233 | 30717 | 27702 | 26008 | 4681 | 5287 | 4302 | C |
| ATOM | 1663 | NH1 | ARG | A | 233 | 94.231 | −80.142 | 122.039 | 1.00 | 208.86 | | N |
| ANISOU | 1663 | NH1 | ARG | A | 233 | 29359 | 25785 | 24212 | 5621 | 6225 | 4864 | N |
| ATOM | 1664 | NH2 | ARG | A | 233 | 95.275 | −81.655 | 123.406 | 1.00 | 208.62 | | N |
| ANISOU | 1664 | NH2 | ARG | A | 233 | 29320 | 26003 | 23942 | 5757 | 5943 | 5087 | N |
| ATOM | 1665 | N | ASP | A | 234 | 86.211 | −80.567 | 121.538 | 1.00 | 177.92 | | N |
| ANISOU | 1665 | N | ASP | A | 234 | 27332 | 18215 | 22057 | 4628 | 10817 | 4699 | N |
| ATOM | 1666 | CA | ASP | A | 234 | 84.799 | −80.675 | 121.934 | 1.00 | 178.34 | | C |
| ANISOU | 1666 | CA | ASP | A | 234 | 27409 | 18020 | 22331 | 4115 | 11068 | 4372 | C |
| ATOM | 1667 | C | ASP | A | 234 | 84.191 | −81.938 | 121.323 | 1.00 | 183.62 | | C |
| ANISOU | 1667 | C | ASP | A | 234 | 27831 | 18501 | 23435 | 3252 | 10887 | 3832 | C |
| ATOM | 1668 | O | ASP | A | 234 | 84.069 | −82.047 | 120.099 | 1.00 | 182.97 | | O |
| ANISOU | 1668 | O | ASP | A | 234 | 27708 | 18061 | 23750 | 2824 | 11121 | 3474 | O |
| ATOM | 1669 | CB | ASP | A | 234 | 83.977 | −79.417 | 121.596 | 1.00 | 180.14 | | C |
| ANISOU | 1669 | CB | ASP | A | 234 | 27561 | 18208 | 22676 | 3820 | 11084 | 4018 | C |
| ATOM | 1670 | CG | ASP | A | 234 | 82.601 | −79.409 | 122.250 | 1.00 | 185.98 | | C |
| ANISOU | 1670 | CG | ASP | A | 234 | 28059 | 19132 | 23472 | 3232 | 10780 | 3592 | C |
| ATOM | 1671 | OD1 | ASP | A | 234 | 81.684 | −80.066 | 121.714 | 1.00 | 185.49 | | O |
| ANISOU | 1671 | OD1 | ASP | A | 234 | 27939 | 18700 | 23840 | 2583 | 11087 | 3154 | O |
| ATOM | 1672 | OD2 | ASP | A | 234 | 82.448 | −78.754 | 123.305 | 1.00 | 190.43 | | O |
| ANISOU | 1672 | OD2 | ASP | A | 234 | 28477 | 20248 | 23631 | 3443 | 10252 | 3696 | O |
| ATOM | 1673 | N | GLU | A | 235 | 83.831 | −82.895 | 122.198 | 1.00 | 181.54 | | N |
| ANISOU | 1673 | N | GLU | A | 235 | 27680 | 18178 | 23120 | 3284 | 11064 | 3994 | N |
| ATOM | 1674 | CA | GLU | A | 235 | 83.278 | −84.209 | 121.862 | 1.00 | 181.85 | | C |
| ANISOU | 1674 | CA | GLU | A | 235 | 27662 | 17938 | 23493 | 2754 | 11218 | 3712 | C |
| ATOM | 1675 | C | GLU | A | 235 | 81.950 | −84.163 | 121.109 | 1.00 | 185.90 | | C |
| ANISOU | 1675 | C | GLU | A | 235 | 27875 | 18389 | 24371 | 1920 | 11137 | 3018 | C |
| ATOM | 1676 | O | GLU | A | 235 | 81.840 | −84.784 | 120.043 | 1.00 | 185.84 | | O |
| ANISOU | 1676 | O | GLU | A | 235 | 27762 | 18158 | 24689 | 1490 | 11245 | 2678 | O |
| ATOM | 1677 | CB | GLU | A | 235 | 83.155 | −85.082 | 123.125 | 1.00 | 183.31 | | C |
| ANISOU | 1677 | CB | GLU | A | 235 | 27842 | 18376 | 23430 | 2882 | 10994 | 3954 | C |
| ATOM | 1678 | N | LEU | A | 236 | 80.943 | −83.451 | 121.673 | 1.00 | 181.79 | | N |
| ANISOU | 1678 | N | LEU | A | 236 | 27501 | 17692 | 23881 | 1892 | 11606 | 2961 | N |
| ATOM | 1679 | CA | LEU | A | 236 | 79.599 | −83.325 | 121.103 | 1.00 | 181.27 | | C |
| ANISOU | 1679 | CA | LEU | A | 236 | 27270 | 17428 | 24177 | 1205 | 11876 | 2360 | C |
| ATOM | 1680 | C | LEU | A | 236 | 79.601 | −82.630 | 119.742 | 1.00 | 185.15 | | C |
| ANISOU | 1680 | C | LEU | A | 236 | 27459 | 18014 | 24877 | 774 | 11591 | 1856 | C |
| ATOM | 1681 | O | LEU | A | 236 | 78.835 | −83.025 | 118.864 | 1.00 | 184.75 | | O |
| ANISOU | 1681 | O | LEU | A | 236 | 27189 | 17854 | 25153 | 159 | 11730 | 1317 | O |
| ATOM | 1682 | CB | LEU | A | 236 | 78.652 | −82.620 | 122.079 | 1.00 | 181.16 | | C |
| ANISOU | 1682 | CB | LEU | A | 236 | 27273 | 17542 | 24018 | 1228 | 11940 | 2351 | C |
| ATOM | 1683 | N | LEU | A | 237 | 80.474 | −81.620 | 119.559 | 1.00 | 181.79 | | N |
| ANISOU | 1683 | N | LEU | A | 237 | 27280 | 17420 | 24373 | 1215 | 11818 | 2124 | N |
| ATOM | 1684 | CA | LEU | A | 237 | 80.581 | −80.902 | 118.294 | 1.00 | 182.04 | | C |
| ANISOU | 1684 | CA | LEU | A | 237 | 27170 | 17336 | 24662 | 872 | 11858 | 1708 | C |
| ATOM | 1685 | C | LEU | A | 237 | 81.115 | −81.825 | 117.208 | 1.00 | 185.29 | | C |
| ANISOU | 1685 | C | LEU | A | 237 | 27354 | 17811 | 25237 | 584 | 11548 | 1499 | C |
| ATOM | 1686 | O | LEU | A | 237 | 80.496 | −81.930 | 116.149 | 1.00 | 185.31 | | O |
| ANISOU | 1686 | O | LEU | A | 237 | 27098 | 17754 | 25555 | −51 | 11627 | 896 | O |
| ATOM | 1687 | CB | LEU | A | 237 | 81.477 | −79.662 | 118.423 | 1.00 | 182.54 | | C |
| ANISOU | 1687 | CB | LEU | A | 237 | 27373 | 17527 | 24458 | 1438 | 11696 | 2076 | C |
| ATOM | 1688 | CG | LEU | A | 237 | 81.573 | −78.798 | 117.163 | 1.00 | 187.80 | | C |
| ANISOU | 1688 | CG | LEU | A | 237 | 27616 | 18492 | 25249 | 985 | 11103 | 1559 | C |
| ATOM | 1689 | CD1 | LEU | A | 237 | 80.547 | −77.705 | 117.182 | 1.00 | 188.24 | | C |
| ANISOU | 1689 | CD1 | LEU | A | 237 | 27259 | 19035 | 25227 | 569 | 10501 | 1088 | C |
| ATOM | 1690 | CD2 | LEU | A | 237 | 82.961 | −78.228 | 116.987 | 1.00 | 190.28 | | C |
| ANISOU | 1690 | CD2 | LEU | A | 237 | 27814 | 19260 | 25225 | 1481 | 10346 | 1918 | C |
| ATOM | 1691 | N | GLN | A | 238 | 82.242 | −82.517 | 117.485 | 1.00 | 180.91 | | N |
| ANISOU | 1691 | N | GLN | A | 238 | 27142 | 17009 | 24586 | 1144 | 11826 | 2060 | N |
| ATOM | 1692 | CA | GLN | A | 238 | 82.861 | −83.439 | 116.539 | 1.00 | 180.29 | | C |
| ANISOU | 1692 | CA | GLN | A | 238 | 27031 | 16759 | 24712 | 985 | 11870 | 1965 | C |
| ATOM | 1693 | C | GLN | A | 238 | 81.944 | −84.597 | 116.150 | 1.00 | 183.17 | | C |
| ANISOU | 1693 | C | GLN | A | 238 | 27103 | 17222 | 25271 | 350 | 11695 | 1499 | C |
| ATOM | 1694 | O | GLN | A | 238 | 82.106 | −85.137 | 115.058 | 1.00 | 182.92 | | O |
| ANISOU | 1694 | O | GLN | A | 238 | 26945 | 17082 | 25473 | 12 | 11731 | 1191 | O |
| ATOM | 1695 | CB | GLN | A | 238 | 84.227 | −83.937 | 117.033 | 1.00 | 181.70 | | C |
| ANISOU | 1695 | CB | GLN | A | 238 | 27345 | 17130 | 24561 | 1651 | 11541 | 2583 | C |
| ATOM | 1696 | CG | GLN | A | 238 | 85.336 | −82.870 | 117.087 | 1.00 | 196.47 | | C |
| ANISOU | 1696 | CG | GLN | A | 238 | 28717 | 20028 | 25905 | 1906 | 10019 | 2670 | C |
| ATOM | 1697 | CD | GLN | A | 238 | 85.475 | −81.972 | 115.873 | 1.00 | 216.14 | | C |
| ANISOU | 1697 | CD | GLN | A | 238 | 30336 | 23544 | 28243 | 1265 | 8284 | 1992 | C |
| ATOM | 1698 | OE1 | GLN | A | 238 | 85.435 | −80.742 | 115.985 | 1.00 | 212.83 | | O |
| ANISOU | 1698 | OE1 | GLN | A | 238 | 30058 | 23007 | 27801 | 1483 | 8580 | 2077 | O |
| ATOM | 1699 | NE2 | GLN | A | 238 | 85.662 | −82.553 | 114.694 | 1.00 | 207.84 | | N |
| ANISOU | 1699 | NE2 | GLN | A | 238 | 29524 | 21836 | 27608 | 1122 | 9107 | 1885 | N |
| ATOM | 1700 | N | SER | A | 239 | 80.956 | −84.941 | 117.011 | 1.00 | 179.08 | | N |
| ANISOU | 1700 | N | SER | A | 239 | 26719 | 16522 | 24800 | 260 | 12130 | 1499 | N |
| ATOM | 1701 | CA | SER | A | 239 | 79.963 | −85.987 | 116.742 | 1.00 | 178.72 | | C |
| ANISOU | 1701 | CA | SER | A | 239 | 26487 | 16445 | 24971 | −280 | 12229 | 1103 | C |
| ATOM | 1702 | C | SER | A | 239 | 79.020 | −85.575 | 115.592 | 1.00 | 183.24 | | C |
| ANISOU | 1702 | C | SER | A | 239 | 26547 | 17371 | 25706 | −973 | 11852 | 364 | C |
| ATOM | 1703 | O | SER | A | 239 | 78.665 | −86.416 | 114.766 | 1.00 | 183.14 | | O |
| ANISOU | 1703 | O | SER | A | 239 | 26330 | 17378 | 25876 | −1379 | 11844 | 14 | O |
| ATOM | 1704 | CB | SER | A | 239 | 79.159 | −86.315 | 118.000 | 1.00 | 181.31 | | C |
| ANISOU | 1704 | CB | SER | A | 239 | 26774 | 17031 | 25083 | −207 | 12013 | 1273 | C |
| ATOM | 1705 | OG | SER | A | 239 | 77.942 | −85.589 | 118.079 | 1.00 | 188.64 | | O |
| ANISOU | 1705 | OG | SER | A | 239 | 27246 | 18529 | 25900 | −583 | 11401 | 865 | O |
| ATOM | 1706 | N | GLN | A | 240 | 78.620 | −84.287 | 115.547 | 1.00 | 179.93 | | N |
| ANISOU | 1706 | N | GLN | A | 240 | 26155 | 16826 | 25386 | −1096 | 12196 | 120 | N |
| ATOM | 1707 | CA | GLN | A | 240 | 77.722 | −83.735 | 114.521 | 1.00 | 179.65 | | C |
| ANISOU | 1707 | CA | GLN | A | 240 | 25728 | 16957 | 25574 | −1755 | 12208 | −630 | C |
| ATOM | 1708 | C | GLN | A | 240 | 78.490 | −83.077 | 113.361 | 1.00 | 183.11 | | C |
| ANISOU | 1708 | C | GLN | A | 240 | 25962 | 17588 | 26024 | −1826 | 11830 | −824 | C |
| ATOM | 1709 | O | GLN | A | 240 | 77.881 | −82.502 | 112.450 | 1.00 | 182.97 | | O |
| ANISOU | 1709 | O | GLN | A | 240 | 25578 | 17797 | 26144 | −2340 | 11777 | −1453 | O |
| ATOM | 1710 | CB | GLN | A | 240 | 76.739 | −82.734 | 115.144 | 1.00 | 180.95 | | C |
| ANISOU | 1710 | CB | GLN | A | 240 | 25752 | 17362 | 25639 | −1867 | 12125 | −814 | C |
| ATOM | 1711 | N | LEU | A | 241 | 79.827 | −83.183 | 113.396 | 1.00 | 178.25 | | N |
| ANISOU | 1711 | N | LEU | A | 241 | 25803 | 16451 | 25474 | −1371 | 12286 | −361 | N |
| ATOM | 1712 | CA | LEU | A | 241 | 80.726 | −82.600 | 112.409 | 1.00 | 176.63 | | C |
| ANISOU | 1712 | CA | LEU | A | 241 | 25618 | 16083 | 25412 | −1384 | 12401 | −474 | C |
| ATOM | 1713 | C | LEU | A | 241 | 81.499 | −83.676 | 111.639 | 1.00 | 177.33 | | C |
| ANISOU | 1713 | C | LEU | A | 241 | 25695 | 16113 | 25569 | −1374 | 12290 | −407 | C |
| ATOM | 1714 | O | LEU | A | 241 | 81.809 | −84.741 | 112.178 | 1.00 | 177.24 | | O |
| ANISOU | 1714 | O | LEU | A | 241 | 25878 | 16001 | 25465 | −1081 | 12295 | −4 | O |
| ATOM | 1715 | CB | LEU | A | 241 | 81.705 | −81.653 | 113.133 | 1.00 | 176.69 | | C |
| ANISOU | 1715 | CB | LEU | A | 241 | 25973 | 15971 | 25189 | −667 | 12402 | 167 | C |
| ATOM | 1716 | CG | LEU | A | 241 | 82.053 | −80.306 | 112.483 | 1.00 | 182.05 | | C |
| ANISOU | 1716 | CG | LEU | A | 241 | 26275 | 17208 | 25690 | −704 | 11565 | −13 | C |
| ATOM | 1717 | CD2 | LEU | A | 241 | 83.240 | −79.676 | 113.181 | 1.00 | 184.72 | | C |
| ANISOU | 1717 | CD2 | LEU | A | 241 | 26735 | 17871 | 25580 | 97 | 10917 | 711 | C |
| ATOM | 1718 | CD1 | LEU | A | 241 | 80.885 | −79.335 | 112.543 | 1.00 | 182.58 | | C |
| ANISOU | 1718 | CD1 | LEU | A | 241 | 25916 | 17807 | 25650 | −1100 | 11050 | −484 | C |
| ATOM | 1719 | N | GLN | A | 242 | 81.809 | −83.375 | 110.373 | 1.00 | 170.12 | | N |
| ANISOU | 1719 | N | GLN | A | 242 | 24775 | 14803 | 25060 | −1794 | 12920 | −914 | N |
| ATOM | 1720 | CA | GLN | A | 242 | 82.580 | −84.213 | 109.458 | 1.00 | 167.69 | | C |
| ANISOU | 1720 | CA | GLN | A | 242 | 24518 | 14263 | 24932 | −1860 | 13102 | −956 | C |
| ATOM | 1721 | C | GLN | A | 242 | 84.026 | −83.684 | 109.497 | 1.00 | 166.71 | | C |
| ANISOU | 1721 | C | GLN | A | 242 | 24733 | 13865 | 24743 | −1225 | 13205 | −372 | C |
| ATOM | 1722 | O | GLN | A | 242 | 84.982 | −84.464 | 109.474 | 1.00 | 166.69 | | O |
| ANISOU | 1722 | O | GLN | A | 242 | 24935 | 13727 | 24674 | −820 | 13179 | 75 | O |
| ATOM | 1723 | CB | GLN | A | 242 | 81.967 | −84.082 | 108.053 | 1.00 | 168.79 | | C |
| ANISOU | 1723 | CB | GLN | A | 242 | 24112 | 14780 | 25240 | −2631 | 12882 | −1830 | C |
| ATOM | 1724 | CG | GLN | A | 242 | 82.518 | −84.982 | 106.965 | 1.00 | 178.27 | | C |
| ANISOU | 1724 | CG | GLN | A | 242 | 24966 | 16540 | 26229 | −2621 | 11929 | −1804 | C |
| ATOM | 1725 | CD | GLN | A | 242 | 81.857 | −84.593 | 105.673 | 1.00 | 191.93 | | C |
| ANISOU | 1725 | CD | GLN | A | 242 | 25875 | 19463 | 27585 | −2936 | 10514 | −2248 | C |
| ATOM | 1726 | OE1 | GLN | A | 242 | 82.216 | −83.596 | 105.039 | 1.00 | 188.78 | | O |
| ANISOU | 1726 | OE1 | GLN | A | 242 | 25393 | 19001 | 27333 | −3126 | 10741 | −2578 | O |
| ATOM | 1727 | NE2 | GLN | A | 242 | 80.843 | −85.344 | 105.279 | 1.00 | 179.70 | | N |
| ANISOU | 1727 | NE2 | GLN | A | 242 | 24291 | 17523 | 26462 | −3628 | 11519 | −2960 | N |
| ATOM | 1728 | N | GLY | A | 243 | 84.141 | −82.359 | 109.598 | 1.00 | 158.69 | | N |
| ANISOU | 1728 | N | GLY | A | 243 | 23464 | 13396 | 23435 | −1059 | 12425 | −310 | N |
| ATOM | 1729 | CA | GLY | A | 243 | 85.390 | −81.608 | 109.664 | 1.00 | 156.19 | | C |
| ANISOU | 1729 | CA | GLY | A | 243 | 23091 | 13521 | 22732 | −434 | 11562 | 233 | C |
| ATOM | 1730 | C | GLY | A | 243 | 85.169 | −80.111 | 109.805 | 1.00 | 153.74 | | C |
| ANISOU | 1730 | C | GLY | A | 243 | 22348 | 14019 | 22046 | −404 | 10449 | 154 | C |
| ATOM | 1731 | O | GLY | A | 243 | 84.027 | −79.636 | 109.774 | 1.00 | 153.07 | | O |
| ANISOU | 1731 | O | GLY | A | 243 | 21986 | 14185 | 21988 | −850 | 10306 | −314 | O |
| ATOM | 1732 | N | PHE | A | 244 | 86.272 | −79.352 | 109.955 | 1.00 | 145.09 | | N |
| ANISOU | 1732 | N | PHE | A | 244 | 21183 | 13342 | 20602 | 122 | 9690 | 592 | N |
| ATOM | 1733 | CA | PHE | A | 244 | 86.247 | −77.895 | 110.117 | 1.00 | 142.16 | | C |
| ANISOU | 1733 | CA | PHE | A | 244 | 20449 | 13680 | 19886 | 202 | 8686 | 563 | C |
| ATOM | 1734 | C | PHE | A | 244 | 87.531 | −77.217 | 109.598 | 1.00 | 137.54 | | C |
| ANISOU | 1734 | C | PHE | A | 244 | 19682 | 13490 | 19087 | 499 | 7954 | 765 | C |
| ATOM | 1735 | O | PHE | A | 244 | 88.548 | −77.888 | 109.426 | 1.00 | 136.92 | | O |
| ANISOU | 1735 | O | PHE | A | 244 | 19807 | 13175 | 19042 | 807 | 8196 | 1080 | O |
| ATOM | 1736 | CB | PHE | A | 244 | 85.950 | −77.512 | 111.579 | 1.00 | 144.30 | | C |
| ANISOU | 1736 | CB | PHE | A | 244 | 20892 | 14065 | 19869 | 604 | 8632 | 937 | C |
| ATOM | 1737 | CG | PHE | A | 244 | 87.043 | −77.831 | 112.572 | 1.00 | 146.41 | | C |
| ANISOU | 1737 | CG | PHE | A | 244 | 21498 | 14281 | 19852 | 1377 | 8742 | 1654 | C |
| ATOM | 1738 | CD2 | PHE | A | 244 | 87.941 | −76.851 | 112.980 | 1.00 | 148.92 | | C |
| ANISOU | 1738 | CD2 | PHE | A | 244 | 21641 | 15186 | 19757 | 1814 | 7954 | 1934 | C |
| ATOM | 1739 | CD1 | PHE | A | 244 | 87.153 | −79.102 | 113.129 | 1.00 | 150.10 | | C |
| ANISOU | 1739 | CD1 | PHE | A | 244 | 22443 | 14147 | 20442 | 1688 | 9679 | 2038 | C |
| ATOM | 1740 | CE2 | PHE | A | 244 | 88.945 | −77.144 | 113.906 | 1.00 | 151.98 | | C |
| ANISOU | 1740 | CE2 | PHE | A | 244 | 22262 | 15675 | 19810 | 2554 | 8034 | 2553 | C |
| ATOM | 1741 | CE1 | PHE | A | 244 | 88.157 | −79.395 | 114.057 | 1.00 | 151.19 | | C |
| ANISOU | 1741 | CE1 | PHE | A | 244 | 22863 | 14341 | 20241 | 2502 | 9784 | 2744 | C |
| ATOM | 1742 | CZ | PHE | A | 244 | 89.043 | −78.412 | 114.443 | 1.00 | 150.24 | | C |
| ANISOU | 1742 | CZ | PHE | A | 244 | 22499 | 14923 | 19661 | 2933 | 8928 | 2982 | C |
| ATOM | 1743 | N | GLU | A | 245 | 87.477 | −75.884 | 109.379 | 1.00 | 127.23 | | N |
| ANISOU | 1743 | N | GLU | A | 245 | 18006 | 12767 | 17569 | 421 | 7112 | 594 | N |
| ATOM | 1744 | CA | GLU | A | 245 | 88.575 | −75.068 | 108.856 | 1.00 | 123.39 | | C |
| ANISOU | 1744 | CA | GLU | A | 245 | 17304 | 12676 | 16901 | 615 | 6411 | 697 | C |
| ATOM | 1745 | C | GLU | A | 245 | 88.604 | −73.704 | 109.569 | 1.00 | 119.74 | | C |
| ANISOU | 1745 | C | GLU | A | 245 | 16661 | 12726 | 16108 | 816 | 5719 | 787 | C |
| ATOM | 1746 | O | GLU | A | 245 | 87.601 | −72.986 | 109.561 | 1.00 | 119.01 | | O |
| ANISOU | 1746 | O | GLU | A | 245 | 16377 | 12833 | 16010 | 531 | 5507 | 487 | O |
| ATOM | 1747 | CB | GLU | A | 245 | 88.372 | −74.899 | 107.341 | 1.00 | 124.46 | | C |
| ANISOU | 1747 | CB | GLU | A | 245 | 17128 | 12916 | 17244 | 125 | 6230 | 210 | C |
| ATOM | 1748 | CG | GLU | A | 245 | 89.437 | −74.105 | 106.607 | 1.00 | 132.51 | | C |
| ANISOU | 1748 | CG | GLU | A | 245 | 17929 | 14280 | 18140 | 253 | 5592 | 264 | C |
| ATOM | 1749 | CD | GLU | A | 245 | 89.212 | −73.916 | 105.115 | 1.00 | 143.24 | | C |
| ANISOU | 1749 | CD | GLU | A | 245 | 18981 | 15789 | 19654 | −173 | 5424 | −181 | C |
| ATOM | 1750 | OE1 | GLU | A | 245 | 89.906 | −73.054 | 104.531 | 1.00 | 127.98 | | O |
| ANISOU | 1750 | OE1 | GLU | A | 245 | 16848 | 14172 | 17606 | −89 | 4874 | −160 | O |
| ATOM | 1751 | OE2 | GLU | A | 245 | 88.359 | −74.618 | 104.524 | 1.00 | 134.31 | | O |
| ANISOU | 1751 | OE2 | GLU | A | 245 | 17794 | 14493 | 18747 | −590 | 5861 | −572 | O |
| ATOM | 1752 | N | VAL | A | 246 | 89.752 | −73.362 | 110.193 | 1.00 | 110.49 | | N |
| ANISOU | 1752 | N | VAL | A | 246 | 15535 | 11793 | 14653 | 1308 | 5404 | 1174 | N |
| ATOM | 1753 | CA | VAL | A | 246 | 89.945 | −72.100 | 110.921 | 1.00 | 107.48 | | C |
| ANISOU | 1753 | CA | VAL | A | 246 | 14982 | 11902 | 13955 | 1493 | 4803 | 1223 | C |
| ATOM | 1754 | C | VAL | A | 246 | 90.931 | −71.186 | 110.174 | 1.00 | 105.70 | | C |
| ANISOU | 1754 | C | VAL | A | 246 | 14484 | 12018 | 13661 | 1484 | 4207 | 1127 | C |
| ATOM | 1755 | O | VAL | A | 246 | 92.147 | −71.374 | 110.262 | 1.00 | 105.49 | | O |
| ANISOU | 1755 | O | VAL | A | 246 | 14473 | 12114 | 13495 | 1817 | 4111 | 1375 | O |
| ATOM | 1756 | CB | VAL | A | 246 | 90.317 | −72.306 | 112.420 | 1.00 | 111.14 | | C |
| ANISOU | 1756 | CB | VAL | A | 246 | 15651 | 12493 | 14084 | 2033 | 4926 | 1652 | C |
| ATOM | 1757 | CG1 | VAL | A | 246 | 90.431 | −70.968 | 113.151 | 1.00 | 110.94 | | C |
| ANISOU | 1757 | CG1 | VAL | A | 246 | 15409 | 12998 | 13745 | 2134 | 4333 | 1589 | C |
| ATOM | 1758 | CG2 | VAL | A | 246 | 89.303 | −73.207 | 113.126 | 1.00 | 110.93 | | C |
| ANISOU | 1758 | CG2 | VAL | A | 246 | 15928 | 12068 | 14151 | 2041 | 5589 | 1764 | C |
| ATOM | 1759 | N | THR | A | 247 | 90.391 | −70.206 | 109.426 | 1.00 | 97.43 | | N |
| ANISOU | 1759 | N | THR | A | 247 | 13183 | 11131 | 12706 | 1123 | 3848 | 773 | N |
| ATOM | 1760 | CA | THR | A | 247 | 91.170 | −69.246 | 108.633 | 1.00 | 94.88 | | C |
| ANISOU | 1760 | CA | THR | A | 247 | 12618 | 11069 | 12362 | 1067 | 3354 | 652 | C |
| ATOM | 1761 | C | THR | A | 247 | 91.273 | −67.902 | 109.332 | 1.00 | 94.68 | | C |
| ANISOU | 1761 | C | THR | A | 247 | 12456 | 11401 | 12118 | 1152 | 2921 | 606 | C |
| ATOM | 1762 | O | THR | A | 247 | 90.353 | −67.512 | 110.035 | 1.00 | 94.35 | | O |
| ANISOU | 1762 | O | THR | A | 247 | 12437 | 11406 | 12008 | 1104 | 2939 | 543 | O |
| ATOM | 1763 | CB | THR | A | 247 | 90.578 | −69.081 | 107.219 | 1.00 | 97.86 | | C |
| ANISOU | 1763 | CB | THR | A | 247 | 12820 | 11389 | 12975 | 672 | 3319 | 324 | C |
| ATOM | 1764 | OG1 | THR | A | 247 | 89.391 | −68.282 | 107.244 | 1.00 | 87.99 | | O |
| ANISOU | 1764 | OG1 | THR | A | 247 | 11435 | 10282 | 11713 | 466 | 3198 | 91 | O |
| ATOM | 1765 | CG2 | THR | A | 247 | 90.322 | −70.396 | 106.525 | 1.00 | 98.17 | | C |
| ANISOU | 1765 | CG2 | THR | A | 247 | 12960 | 11095 | 13246 | 493 | 3798 | 250 | C |
| ATOM | 1766 | N | GLY | A | 248 | 92.368 | −67.192 | 109.114 | 1.00 | 88.74 | | N |
| ANISOU | 1766 | N | GLY | A | 248 | 11556 | 10885 | 11277 | 1242 | 2573 | 600 | N |
| ATOM | 1767 | CA | GLY | A | 248 | 92.577 | −65.886 | 109.727 | 1.00 | 87.00 | | C |
| ANISOU | 1767 | CA | GLY | A | 248 | 11193 | 10980 | 10883 | 1268 | 2224 | 485 | C |
| ATOM | 1768 | C | GLY | A | 248 | 92.854 | −64.784 | 108.739 | 1.00 | 86.02 | | C |
| ANISOU | 1768 | C | GLY | A | 248 | 10882 | 10915 | 10887 | 1053 | 1949 | 260 | C |
| ATOM | 1769 | O | GLY | A | 248 | 92.941 | −65.048 | 107.539 | 1.00 | 85.65 | | O |
| ANISOU | 1769 | O | GLY | A | 248 | 10796 | 10717 | 11029 | 918 | 1983 | 217 | O |
| ATOM | 1770 | N | ARG | A | 249 | 92.978 | −63.539 | 109.232 | 1.00 | 78.99 | | N |
| ANISOU | 1770 | N | ARG | A | 249 | 9884 | 10231 | 9898 | 1023 | 1725 | 107 | N |
| ATOM | 1771 | CA | ARG | A | 249 | 93.240 | −62.370 | 108.388 | 1.00 | 77.14 | | C |
| ANISOU | 1771 | CA | ARG | A | 249 | 9515 | 9997 | 9797 | 846 | 1555 | −89 | C |
| ATOM | 1772 | C | ARG | A | 249 | 93.603 | −61.230 | 109.327 | 1.00 | 78.37 | | C |
| ANISOU | 1772 | C | ARG | A | 249 | 9589 | 10352 | 9837 | 821 | 1417 | −274 | C |
| ATOM | 1773 | O | ARG | A | 249 | 92.854 | −60.995 | 110.271 | 1.00 | 79.34 | | O |
| ANISOU | 1773 | O | ARG | A | 249 | 9761 | 10460 | 9926 | 781 | 1462 | −338 | O |
| ATOM | 1774 | CB | ARG | A | 249 | 92.018 | −62.091 | 107.456 | 1.00 | 75.93 | | C |
| ANISOU | 1774 | CB | ARG | A | 249 | 9367 | 9661 | 9820 | 677 | 1642 | −158 | C |
| ATOM | 1775 | CG | ARG | A | 249 | 92.181 | −61.036 | 106.344 | 1.00 | 80.47 | | C |
| ANISOU | 1775 | CG | ARG | A | 249 | 9843 | 10197 | 10537 | 575 | 1557 | −269 | C |
| ATOM | 1776 | CD | ARG | A | 249 | 91.611 | −59.646 | 106.649 | 1.00 | 77.93 | | C |
| ANISOU | 1776 | CD | ARG | A | 249 | 9513 | 9883 | 10215 | 544 | 1549 | −388 | C |
| ATOM | 1777 | NE | ARG | A | 249 | 90.171 | −59.642 | 106.921 | 1.00 | 78.04 | | N |
| ANISOU | 1777 | NE | ARG | A | 249 | 9561 | 9901 | 10190 | 548 | 1646 | −384 | N |
| ATOM | 1778 | CZ | ARG | A | 249 | 89.591 | −58.976 | 107.922 | 1.00 | 82.70 | | C |
| ANISOU | 1778 | CZ | ARG | A | 249 | 10198 | 10526 | 10699 | 565 | 1670 | −445 | C |
| ATOM | 1779 | NH1 | ARG | A | 249 | 90.324 | −58.250 | 108.762 | 1.00 | 53.97 | | N |
| ANISOU | 1779 | NH1 | ARG | A | 249 | 6569 | 6936 | 7002 | 557 | 1611 | −551 | N |
| ATOM | 1780 | NH2 | ARG | A | 249 | 88.276 | −59.040 | 108.098 | 1.00 | 67.04 | | N |
| ANISOU | 1780 | NH2 | ARG | A | 249 | 8224 | 8565 | 8681 | 573 | 1764 | −441 | N |
| ATOM | 1781 | N | PRO | A | 250 | 94.642 | −60.418 | 109.071 | 1.00 | 71.71 | | N |
| ANISOU | 1781 | N | PRO | A | 250 | 8602 | 9686 | 8957 | 798 | 1281 | −418 | N |
| ATOM | 1782 | CA | PRO | A | 250 | 94.820 | −59.206 | 109.902 | 1.00 | 70.74 | | C |
| ANISOU | 1782 | CA | PRO | A | 250 | 8389 | 9703 | 8785 | 677 | 1229 | −699 | C |
| ATOM | 1783 | CB | PRO | A | 250 | 96.322 | −59.064 | 110.129 | 1.00 | 72.87 | | C |
| ANISOU | 1783 | CB | PRO | A | 250 | 8454 | 10297 | 8937 | 684 | 1099 | −864 | C |
| ATOM | 1784 | CG | PRO | A | 250 | 96.958 | −59.918 | 109.072 | 1.00 | 77.35 | | C |
| ANISOU | 1784 | CG | PRO | A | 250 | 9020 | 10756 | 9615 | 753 | 1083 | −674 | C |
| ATOM | 1785 | CD | PRO | A | 250 | 95.954 | −60.950 | 108.601 | 1.00 | 72.87 | | C |
| ANISOU | 1785 | CD | PRO | A | 250 | 8649 | 9907 | 9132 | 842 | 1207 | −383 | C |
| ATOM | 1786 | C | PRO | A | 250 | 94.302 | −58.130 | 108.931 | 1.00 | 71.85 | | C |
| ANISOU | 1786 | C | PRO | A | 250 | 8570 | 9537 | 9192 | 492 | 1326 | −804 | C |
| ATOM | 1787 | O | PRO | A | 250 | 94.512 | −58.271 | 107.723 | 1.00 | 72.40 | | O |
| ANISOU | 1787 | O | PRO | A | 250 | 8642 | 9435 | 9433 | 463 | 1348 | −721 | O |
| ATOM | 1788 | N | LYS | A | 251 | 93.600 | −57.095 | 109.404 | 1.00 | 65.67 | | N |
| ANISOU | 1788 | N | LYS | A | 251 | 7831 | 8687 | 8434 | 409 | 1417 | −952 | N |
| ATOM | 1789 | CA | LYS | A | 251 | 93.092 | −56.100 | 108.454 | 1.00 | 64.09 | | C |
| ANISOU | 1789 | CA | LYS | A | 251 | 7696 | 8192 | 8463 | 332 | 1576 | −974 | C |
| ATOM | 1790 | C | LYS | A | 251 | 94.223 | −55.255 | 107.889 | 1.00 | 67.96 | | C |
| ANISOU | 1790 | C | LYS | A | 251 | 8116 | 8594 | 9113 | 191 | 1646 | −1163 | C |
| ATOM | 1791 | O | LYS | A | 251 | 95.108 | −54.831 | 108.640 | 1.00 | 67.90 | | O |
| ANISOU | 1791 | O | LYS | A | 251 | 7993 | 8765 | 9042 | 61 | 1626 | −1443 | O |
| ATOM | 1792 | CB | LYS | A | 251 | 91.976 | −55.240 | 109.046 | 1.00 | 64.60 | | C |
| ANISOU | 1792 | CB | LYS | A | 251 | 7853 | 8164 | 8526 | 329 | 1718 | −1038 | C |
| ATOM | 1793 | CG | LYS | A | 251 | 90.974 | −54.828 | 107.985 | 1.00 | 56.66 | | C |
| ANISOU | 1793 | CG | LYS | A | 251 | 6928 | 6941 | 7661 | 423 | 1867 | −871 | C |
| ATOM | 1794 | CD | LYS | A | 251 | 90.081 | −53.691 | 108.463 | 1.00 | 54.15 | | C |
| ANISOU | 1794 | CD | LYS | A | 251 | 6705 | 6497 | 7375 | 447 | 2072 | −942 | C |
| ATOM | 1795 | CE | LYS | A | 251 | 88.904 | −53.422 | 107.555 | 1.00 | 46.25 | | C |
| ANISOU | 1795 | CE | LYS | A | 251 | 5745 | 5414 | 6414 | 643 | 2203 | −729 | C |
| ATOM | 1796 | NZ | LYS | A | 251 | 87.949 | −54.569 | 107.505 | 1.00 | 48.92 | | N |
| ANISOU | 1796 | NZ | LYS | A | 251 | 6013 | 5963 | 6612 | 718 | 2063 | −596 | N |
| ATOM | 1797 | N | HIS | A | 252 | 94.237 | −55.074 | 106.561 | 1.00 | 63.88 | | N |
| ANISOU | 1797 | N | HIS | A | 252 | 7641 | 7852 | 8779 | 217 | 1740 | −1032 | N |
| ATOM | 1798 | CA | HIS | A | 252 | 95.308 | −54.313 | 105.935 | 1.00 | 63.20 | | C |
| ANISOU | 1798 | CA | HIS | A | 252 | 7512 | 7630 | 8871 | 85 | 1864 | −1188 | C |
| ATOM | 1799 | CB | HIS | A | 252 | 95.764 | −54.924 | 104.605 | 1.00 | 64.30 | | C |
| ANISOU | 1799 | CB | HIS | A | 252 | 7628 | 7705 | 9097 | 155 | 1811 | −993 | C |
| ATOM | 1800 | CG | HIS | A | 252 | 94.789 | −54.766 | 103.485 | 1.00 | 68.13 | | C |
| ANISOU | 1800 | CG | HIS | A | 252 | 8215 | 8020 | 9653 | 322 | 1934 | −745 | C |
| ATOM | 1801 | ND1 | HIS | A | 252 | 94.863 | −53.696 | 102.611 | 1.00 | 70.07 | | N |
| ANISOU | 1801 | ND1 | HIS | A | 252 | 8545 | 8010 | 10069 | 365 | 2207 | −708 | N |
| ATOM | 1802 | CE1 | HIS | A | 252 | 93.885 | −53.874 | 101.739 | 1.00 | 69.54 | | C |
| ANISOU | 1802 | CE1 | HIS | A | 252 | 8507 | 7971 | 9945 | 590 | 2234 | −456 | C |
| ATOM | 1803 | NE2 | HIS | A | 252 | 93.199 | −54.985 | 101.994 | 1.00 | 69.94 | | N |
| ANISOU | 1803 | NE2 | HIS | A | 252 | 8482 | 8258 | 9833 | 625 | 2011 | −391 | N |
| ATOM | 1804 | CD2 | HIS | A | 252 | 93.764 | −55.564 | 103.111 | 1.00 | 70.22 | | C |
| ANISOU | 1804 | CD2 | HIS | A | 252 | 8480 | 8389 | 9812 | 468 | 1846 | −544 | C |
| ATOM | 1805 | C | HIS | A | 252 | 94.943 | −52.858 | 105.836 | 1.00 | 65.38 | | C |
| ANISOU | 1805 | C | HIS | A | 252 | 7910 | 7605 | 9326 | 24 | 2200 | −1304 | C |
| ATOM | 1806 | O | HIS | A | 252 | 93.771 | −52.515 | 105.677 | 1.00 | 63.48 | | O |
| ANISOU | 1806 | O | HIS | A | 252 | 7797 | 7231 | 9093 | 179 | 2326 | −1133 | O |
| ATOM | 1807 | N | LEU | A | 253 | 95.968 | −52.006 | 105.989 | 1.00 | 63.21 | | N |
| ANISOU | 1807 | N | LEU | A | 253 | 7582 | 7241 | 9192 | −205 | 2381 | −1624 | N |
| ATOM | 1808 | CA | LEU | A | 253 | 95.926 | −50.541 | 106.027 | 1.00 | 63.21 | | C |
| ANISOU | 1808 | CA | LEU | A | 253 | 7709 | 6891 | 9417 | −340 | 2819 | −1835 | C |
| ATOM | 1809 | CB | LEU | A | 253 | 97.320 | −49.941 | 105.849 | 1.00 | 62.40 | | C |
| ANISOU | 1809 | CB | LEU | A | 253 | 7500 | 6709 | 9501 | −638 | 3017 | −2200 | C |
| ATOM | 1810 | CG | LEU | A | 253 | 98.240 | −50.085 | 107.039 | 1.00 | 66.65 | | C |
| ANISOU | 1810 | CG | LEU | A | 253 | 7765 | 7677 | 9883 | −917 | 2841 | −2674 | C |
| ATOM | 1811 | CD1 | LEU | A | 253 | 99.468 | −49.349 | 106.835 | 1.00 | 67.77 | | C |
| ANISOU | 1811 | CD1 | LEU | A | 253 | 7778 | 7742 | 10229 | −1249 | 3107 | −3101 | C |
| ATOM | 1812 | CD2 | LEU | A | 253 | 97.668 | −49.497 | 108.235 | 1.00 | 69.35 | | C |
| ANISOU | 1812 | CD2 | LEU | A | 253 | 8120 | 8084 | 10145 | −1025 | 2947 | −2935 | C |
| ATOM | 1813 | C | LEU | A | 253 | 94.951 | −49.898 | 105.061 | 1.00 | 69.19 | | C |
| ANISOU | 1813 | C | LEU | A | 253 | 8700 | 7274 | 10316 | −86 | 3127 | −1504 | C |
| ATOM | 1814 | O | LEU | A | 253 | 94.146 | −49.067 | 105.499 | 1.00 | 68.62 | | O |
| ANISOU | 1814 | O | LEU | A | 253 | 8772 | 7010 | 10291 | −34 | 3396 | −1522 | O |
| ATOM | 1815 | N | TYR | A | 254 | 94.979 | −50.305 | 103.768 | 1.00 | 65.99 | | N |
| ANISOU | 1815 | N | TYR | A | 254 | 8316 | 6818 | 9940 | 113 | 3090 | −1190 | N |
| ATOM | 1816 | CA | TYR | A | 254 | 94.039 | −49.770 | 102.791 | 1.00 | 65.06 | | C |
| ANISOU | 1816 | CA | TYR | A | 254 | 8369 | 6486 | 9866 | 445 | 3355 | −837 | C |
| ATOM | 1817 | CB | TYR | A | 254 | 94.363 | −50.222 | 101.371 | 1.00 | 64.57 | | C |
| ANISOU | 1817 | CB | TYR | A | 254 | 8276 | 6450 | 9809 | 621 | 3305 | −573 | C |
| ATOM | 1818 | CG | TYR | A | 254 | 93.623 | −49.417 | 100.332 | 1.00 | 64.34 | | C |
| ANISOU | 1818 | CG | TYR | A | 254 | 8412 | 6227 | 9808 | 1004 | 3672 | −231 | C |
| ATOM | 1819 | CD1 | TYR | A | 254 | 93.827 | −48.047 | 100.209 | 1.00 | 66.69 | | C |
| ANISOU | 1819 | CD1 | TYR | A | 254 | 8935 | 6063 | 10340 | 1044 | 4240 | −241 | C |
| ATOM | 1820 | CE1 | TYR | A | 254 | 93.171 | −47.301 | 99.231 | 1.00 | 68.91 | | C |
| ANISOU | 1820 | CE1 | TYR | A | 254 | 9391 | 6176 | 10615 | 1498 | 4636 | 150 | C |
| ATOM | 1821 | CZ | TYR | A | 254 | 92.297 | −47.931 | 98.356 | 1.00 | 77.06 | | C |
| ANISOU | 1821 | CZ | TYR | A | 254 | 10307 | 7614 | 11360 | 1902 | 4401 | 507 | C |
| ATOM | 1822 | OH | TYR | A | 254 | 91.652 | −47.192 | 97.386 | 1.00 | 81.99 | | O |
| ANISOU | 1822 | OH | TYR | A | 254 | 11063 | 8190 | 11901 | 2431 | 4786 | 917 | O |
| ATOM | 1823 | CE2 | TYR | A | 254 | 92.068 | −49.294 | 98.472 | 1.00 | 65.18 | | C |
| ANISOU | 1823 | CE2 | TYR | A | 254 | 8543 | 6585 | 9637 | 1786 | 3827 | 437 | C |
| ATOM | 1824 | CD2 | TYR | A | 254 | 92.720 | −50.023 | 99.465 | 1.00 | 64.30 | | C |
| ANISOU | 1824 | CD2 | TYR | A | 254 | 8325 | 6529 | 9578 | 1348 | 3498 | 94 | C |
| ATOM | 1825 | C | TYR | A | 254 | 92.589 | −50.069 | 103.176 | 1.00 | 71.32 | | C |
| ANISOU | 1825 | C | TYR | A | 254 | 9175 | 7469 | 10456 | 675 | 3224 | −647 | C |
| ATOM | 1826 | O | TYR | A | 254 | 91.795 | −49.139 | 103.200 | 1.00 | 70.72 | | O |
| ANISOU | 1826 | O | TYR | A | 254 | 9250 | 7196 | 10423 | 861 | 3549 | −541 | O |
| ATOM | 1827 | N | SER | A | 255 | 92.267 | −51.335 | 103.557 | 1.00 | 69.85 | | N |
| ANISOU | 1827 | N | SER | A | 255 | 8836 | 7641 | 10061 | 653 | 2803 | −624 | N |
| ATOM | 1828 | CA | SER | A | 255 | 90.924 | −51.760 | 103.974 | 1.00 | 70.17 | | C |
| ANISOU | 1828 | CA | SER | A | 255 | 8855 | 7890 | 9915 | 810 | 2678 | −500 | C |
| ATOM | 1829 | CB | SER | A | 255 | 90.823 | −53.276 | 104.051 | 1.00 | 73.02 | | C |
| ANISOU | 1829 | CB | SER | A | 255 | 9067 | 8564 | 10112 | 760 | 2310 | −468 | C |
| ATOM | 1830 | OG | SER | A | 255 | 90.773 | −53.818 | 102.744 | 1.00 | 82.75 | | O |
| ANISOU | 1830 | OG | SER | A | 255 | 10220 | 9901 | 11321 | 893 | 2254 | −288 | O |
| ATOM | 1831 | C | SER | A | 255 | 90.535 | −51.157 | 105.299 | 1.00 | 77.46 | | C |
| ANISOU | 1831 | C | SER | A | 255 | 9854 | 8747 | 10831 | 707 | 2778 | −691 | C |
| ATOM | 1832 | O | SER | A | 255 | 89.337 | −51.019 | 105.552 | 1.00 | 77.95 | | O |
| ANISOU | 1832 | O | SER | A | 255 | 9948 | 8874 | 10793 | 878 | 2825 | −577 | O |
| ATOM | 1833 | N | ILE | A | 256 | 91.535 | −50.792 | 106.148 | 1.00 | 76.28 | | N |
| ANISOU | 1833 | N | ILE | A | 256 | 9698 | 8521 | 10765 | 423 | 2815 | −1014 | N |
| ATOM | 1834 | CA | ILE | A | 256 | 91.318 | −50.157 | 107.463 | 1.00 | 77.30 | | C |
| ANISOU | 1834 | CA | ILE | A | 256 | 9870 | 8624 | 10877 | 275 | 2929 | −1281 | C |
| ATOM | 1835 | CB | ILE | A | 256 | 92.565 | −50.219 | 108.406 | 1.00 | 79.83 | | C |
| ANISOU | 1835 | CB | ILE | A | 256 | 10047 | 9115 | 11169 | −52 | 2818 | −1692 | C |
| ATOM | 1836 | CG1 | ILE | A | 256 | 93.049 | −51.641 | 108.688 | 1.00 | 80.50 | | C |
| ANISOU | 1836 | CG1 | ILE | A | 256 | 9957 | 9603 | 11028 | −44 | 2383 | −1638 | C |
| ATOM | 1837 | CD1 | ILE | A | 256 | 94.460 | −51.717 | 109.268 | 1.00 | 89.54 | | C |
| ANISOU | 1837 | CD1 | ILE | A | 256 | 10903 | 11007 | 12111 | −277 | 2273 | −1991 | C |
| ATOM | 1838 | CG2 | ILE | A | 256 | 92.308 | −49.482 | 109.713 | 1.00 | 78.99 | | C |
| ANISOU | 1838 | CG2 | ILE | A | 256 | 9961 | 9028 | 11024 | −213 | 2966 | −2016 | C |
| ATOM | 1839 | C | ILE | A | 256 | 90.907 | −48.706 | 107.225 | 1.00 | 86.14 | | C |
| ANISOU | 1839 | C | ILE | A | 256 | 11190 | 9337 | 12203 | 353 | 3431 | −1281 | C |
| ATOM | 1840 | O | ILE | A | 256 | 89.964 | −48.208 | 107.837 | 1.00 | 86.27 | | O |
| ANISOU | 1840 | O | ILE | A | 256 | 11302 | 9293 | 12186 | 442 | 3581 | −1272 | O |
| ATOM | 1841 | N | TRP | A | 257 | 91.627 | −48.030 | 106.343 | 1.00 | 86.58 | | N |
| ANISOU | 1841 | N | TRP | A | 257 | 11328 | 9088 | 12481 | 334 | 3738 | −1278 | N |
| ATOM | 1842 | CA | TRP | A | 257 | 91.373 | −46.639 | 106.030 | 1.00 | 88.30 | | C |
| ANISOU | 1842 | CA | TRP | A | 257 | 11784 | 8833 | 12932 | 436 | 4333 | −1245 | C |
| ATOM | 1843 | CB | TRP | A | 257 | 92.625 | −46.050 | 105.401 | 1.00 | 88.65 | | C |
| ANISOU | 1843 | CB | TRP | A | 257 | 11879 | 8559 | 13247 | 236 | 4658 | −1417 | C |
| ATOM | 1844 | CG | TRP | A | 257 | 92.474 | −44.642 | 104.945 | 1.00 | 91.31 | | C |
| ANISOU | 1844 | CG | TRP | A | 257 | 12509 | 8316 | 13867 | 361 | 5387 | −1345 | C |
| ATOM | 1845 | CD1 | TRP | A | 257 | 92.529 | −43.517 | 105.717 | 1.00 | 95.02 | | C |
| ANISOU | 1845 | CD1 | TRP | A | 257 | 13137 | 8406 | 14561 | 133 | 5908 | −1686 | C |
| ATOM | 1846 | NE1 | TRP | A | 257 | 92.366 | −42.399 | 104.929 | 1.00 | 95.34 | | N |
| ANISOU | 1846 | NE1 | TRP | A | 257 | 13492 | 7872 | 14862 | 380 | 6622 | −1454 | N |
| ATOM | 1847 | CE2 | TRP | A | 257 | 92.184 | −42.793 | 103.628 | 1.00 | 96.52 | | C |
| ANISOU | 1847 | CE2 | TRP | A | 257 | 13665 | 8098 | 14909 | 812 | 6523 | −935 | C |
| ATOM | 1848 | CD2 | TRP | A | 257 | 92.238 | −44.203 | 103.604 | 1.00 | 91.49 | | C |
| ANISOU | 1848 | CD2 | TRP | A | 257 | 12722 | 8065 | 13976 | 776 | 5741 | −891 | C |
| ATOM | 1849 | CE3 | TRP | A | 257 | 92.052 | −44.865 | 102.380 | 1.00 | 92.64 | | C |
| ANISOU | 1849 | CE3 | TRP | A | 257 | 12803 | 8438 | 13957 | 1141 | 5505 | −454 | C |
| ATOM | 1850 | CZ3 | TRP | A | 257 | 91.825 | −44.117 | 101.240 | 1.00 | 94.14 | | C |
| ANISOU | 1850 | CZ3 | TRP | A | 257 | 13213 | 8326 | 14230 | 1574 | 6003 | −49 | C |
| ATOM | 1851 | CH2 | TRP | A | 257 | 91.763 | −42.724 | 101.296 | 1.00 | 95.03 | | C |
| ANISOU | 1851 | CH2 | TRP | A | 257 | 13652 | 7834 | 14620 | 1670 | 6790 | −32 | C |
| ATOM | 1852 | CZ2 | TRP | A | 257 | 91.940 | −42.039 | 102.479 | 1.00 | 95.83 | | C |
| ANISOU | 1852 | CZ2 | TRP | A | 257 | 13846 | 7624 | 14940 | 1269 | 7086 | −487 | C |
| ATOM | 1853 | C | TRP | A | 257 | 90.136 | −46.453 | 105.151 | 1.00 | 91.05 | | C |
| ANISOU | 1853 | C | TRP | A | 257 | 12254 | 9143 | 13199 | 943 | 4476 | −750 | C |
| ATOM | 1854 | O | TRP | A | 257 | 89.434 | −45.458 | 105.299 | 1.00 | 91.12 | | O |
| ANISOU | 1854 | O | TRP | A | 257 | 12457 | 8873 | 13290 | 1134 | 4907 | −660 | O |
| ATOM | 1855 | N | LYS | A | 258 | 89.876 | −47.436 | 104.284 | 1.00 | 86.65 | | N |
| ANISOU | 1855 | N | LYS | A | 258 | 11557 | 8911 | 12456 | 1164 | 4128 | −457 | N |
| ATOM | 1856 | CA | LYS | A | 258 | 88.721 | −47.402 | 103.348 | 1.00 | 85.95 | | C |
| ANISOU | 1856 | CA | LYS | A | 258 | 11462 | 9000 | 12194 | 1650 | 4161 | −33 | C |
| ATOM | 1857 | CB | LYS | A | 258 | 88.775 | −48.603 | 102.398 | 1.00 | 88.46 | | C |
| ANISOU | 1857 | CB | LYS | A | 258 | 11548 | 9740 | 12323 | 1694 | 3710 | 93 | C |
| ATOM | 1858 | CG | LYS | A | 258 | 88.330 | −48.325 | 100.968 | 1.00 | 91.77 | | C |
| ANISOU | 1858 | CG | LYS | A | 258 | 11959 | 10224 | 12686 | 2067 | 3856 | 427 | C |
| ATOM | 1859 | CD | LYS | A | 258 | 88.640 | −49.459 | 100.015 | 1.00 | 98.58 | | C |
| ANISOU | 1859 | CD | LYS | A | 258 | 12586 | 11457 | 13413 | 1963 | 3417 | 415 | C |
| ATOM | 1860 | CE | LYS | A | 258 | 87.440 | −50.336 | 99.724 | 1.00 | 100.88 | | C |
| ANISOU | 1860 | CE | LYS | A | 258 | 12645 | 12286 | 13400 | 2219 | 3183 | 567 | C |
| ATOM | 1861 | NZ | LYS | A | 258 | 87.803 | −51.500 | 98.883 | 1.00 | 103.24 | | N |
| ANISOU | 1861 | NZ | LYS | A | 258 | 12752 | 12851 | 13622 | 2114 | 2895 | 538 | N |
| ATOM | 1862 | C | LYS | A | 258 | 87.401 | −47.379 | 104.129 | 1.00 | 87.42 | | C |
| ANISOU | 1862 | C | LYS | A | 258 | 11635 | 9366 | 12213 | 1814 | 4124 | 23 | C |
| ATOM | 1863 | O | LYS | A | 258 | 86.537 | −46.549 | 103.788 | 1.00 | 85.75 | | O |
| ANISOU | 1863 | O | LYS | A | 258 | 11534 | 9082 | 11964 | 2214 | 4460 | 288 | O |
| ATOM | 1864 | N | LYS | A | 259 | 87.251 | −48.255 | 105.129 | 1.00 | 84.07 | | N |
| ANISOU | 1864 | N | LYS | A | 259 | 11081 | 9185 | 11677 | 1541 | 3748 | −206 | N |
| ATOM | 1865 | CA | LYS | A | 259 | 86.010 | −48.308 | 105.894 | 1.00 | 84.45 | | C |
| ANISOU | 1865 | CA | LYS | A | 259 | 11102 | 9418 | 11567 | 1654 | 3693 | −182 | C |
| ATOM | 1866 | C | LYS | A | 259 | 85.920 | −47.230 | 106.961 | 1.00 | 89.51 | | C |
| ANISOU | 1866 | C | LYS | A | 259 | 11934 | 9728 | 12348 | 1547 | 4032 | −376 | C |
| ATOM | 1867 | O | LYS | A | 259 | 84.798 | −46.885 | 107.348 | 1.00 | 89.61 | | O |
| ANISOU | 1867 | O | LYS | A | 259 | 11983 | 9797 | 12267 | 1760 | 4148 | −274 | O |
| ATOM | 1868 | CB | LYS | A | 259 | 85.687 | −49.698 | 106.452 | 1.00 | 86.55 | | C |
| ANISOU | 1868 | CB | LYS | A | 259 | 11168 | 10079 | 11638 | 1480 | 3224 | −279 | C |
| ATOM | 1869 | CG | LYS | A | 259 | 85.010 | −50.606 | 105.414 | 1.00 | 97.89 | | C |
| ANISOU | 1869 | CG | LYS | A | 259 | 12400 | 11904 | 12888 | 1690 | 3015 | −71 | C |
| ATOM | 1870 | CD | LYS | A | 259 | 83.864 | −49.981 | 104.623 | 1.00 | 104.89 | | C |
| ANISOU | 1870 | CD | LYS | A | 259 | 13243 | 12969 | 13643 | 2143 | 3226 | 198 | C |
| ATOM | 1871 | CE | LYS | A | 259 | 83.416 | −50.819 | 103.457 | 1.00 | 115.09 | | C |
| ANISOU | 1871 | CE | LYS | A | 259 | 14272 | 14729 | 14730 | 2318 | 3031 | 323 | C |
| ATOM | 1872 | NZ | LYS | A | 259 | 82.387 | −50.134 | 102.639 | 1.00 | 122.52 | | N |
| ANISOU | 1872 | NZ | LYS | A | 259 | 15121 | 15958 | 15473 | 2835 | 3241 | 593 | N |
| ATOM | 1873 | N | MET | A | 260 | 87.045 | −46.635 | 107.386 | 1.00 | 85.89 | | N |
| ANISOU | 1873 | N | MET | A | 260 | 11582 | 8941 | 12112 | 1226 | 4237 | −677 | N |
| ATOM | 1874 | CA | MET | A | 260 | 86.963 | −45.520 | 108.327 | 1.00 | 85.85 | | C |
| ANISOU | 1874 | CA | MET | A | 260 | 11753 | 8603 | 12263 | 1092 | 4644 | −925 | C |
| ATOM | 1875 | CB | MET | A | 260 | 88.343 | −45.125 | 108.827 | 1.00 | 88.14 | | C |
| ANISOU | 1875 | CB | MET | A | 260 | 12049 | 8684 | 12757 | 627 | 4783 | −1389 | C |
| ATOM | 1876 | CG | MET | A | 260 | 88.797 | −45.950 | 109.962 | 1.00 | 91.78 | | C |
| ANISOU | 1876 | CG | MET | A | 260 | 12298 | 9534 | 13038 | 289 | 4334 | −1732 | C |
| ATOM | 1877 | SD | MET | A | 260 | 90.231 | −45.196 | 110.700 | 1.00 | 96.12 | | S |
| ANISOU | 1877 | SD | MET | A | 260 | 12798 | 9943 | 13782 | −240 | 4591 | −2378 | S |
| ATOM | 1878 | CE | MET | A | 260 | 91.472 | −45.861 | 109.697 | 1.00 | 92.68 | | C |
| ANISOU | 1878 | CE | MET | A | 260 | 12216 | 9610 | 13390 | −322 | 4367 | −2336 | C |
| ATOM | 1879 | C | MET | A | 260 | 86.295 | −44.300 | 107.697 | 1.00 | 90.53 | | C |
| ANISOU | 1879 | C | MET | A | 260 | 12594 | 8808 | 12995 | 1486 | 5243 | −638 | C |
| ATOM | 1880 | O | MET | A | 260 | 85.568 | −43.583 | 108.375 | 1.00 | 88.50 | | O |
| ANISOU | 1880 | O | MET | A | 260 | 12472 | 8385 | 12770 | 1568 | 5537 | −673 | O |
| ATOM | 1881 | N | GLU | A | 261 | 86.573 | −44.065 | 106.401 | 1.00 | 89.96 | | N |
| ANISOU | 1881 | N | GLU | A | 261 | 12591 | 8593 | 12997 | 1761 | 5453 | −335 | N |
| ATOM | 1882 | CA | GLU | A | 261 | 86.042 | −42.964 | 105.582 | 1.00 | 90.89 | | C |
| ANISOU | 1882 | CA | GLU | A | 261 | 12957 | 8368 | 13210 | 2259 | 6068 | 48 | C |
| ATOM | 1883 | CB | GLU | A | 261 | 86.943 | −42.683 | 104.347 | 1.00 | 92.50 | | C |
| ANISOU | 1883 | CB | GLU | A | 261 | 13259 | 8315 | 13571 | 2369 | 6344 | 225 | C |
| ATOM | 1884 | CG | GLU | A | 261 | 88.429 | −42.479 | 104.618 | 1.00 | 103.40 | | C |
| ANISOU | 1884 | CG | GLU | A | 261 | 14685 | 9342 | 15261 | 1802 | 6483 | −230 | C |
| ATOM | 1885 | CD | GLU | A | 261 | 88.873 | −41.079 | 104.993 | 1.00 | 125.93 | | C |
| ANISOU | 1885 | CD | GLU | A | 261 | 17853 | 11492 | 18501 | 1620 | 7290 | −476 | C |
| ATOM | 1886 | OE1 | GLU | A | 261 | 89.387 | −40.352 | 104.108 | 1.00 | 121.52 | | O |
| ANISOU | 1886 | OE1 | GLU | A | 261 | 17519 | 10477 | 18178 | 1767 | 7860 | −312 | O |
| ATOM | 1887 | OE2 | GLU | A | 261 | 88.710 | −40.710 | 106.179 | 1.00 | 114.89 | | O |
| ANISOU | 1887 | OE2 | GLU | A | 261 | 16484 | 9991 | 17178 | 1318 | 7394 | −854 | O |
| ATOM | 1888 | C | GLU | A | 261 | 84.606 | −43.281 | 105.120 | 1.00 | 95.71 | | C |
| ANISOU | 1888 | C | GLU | A | 261 | 13448 | 9430 | 13488 | 2824 | 5899 | 487 | C |
| ATOM | 1889 | O | GLU | A | 261 | 83.733 | −42.416 | 105.198 | 1.00 | 96.98 | | O |
| ANISOU | 1889 | O | GLU | A | 261 | 13774 | 9435 | 13640 | 3212 | 6327 | 711 | O |
| ATOM | 1890 | N | ARG | A | 262 | 84.377 | −44.527 | 104.655 | 1.00 | 91.17 | | N |
| ANISOU | 1890 | N | ARG | A | 262 | 12568 | 9433 | 12639 | 2855 | 5306 | 571 | N |
| ATOM | 1891 | CA | ARG | A | 262 | 83.109 | −45.052 | 104.151 | 1.00 | 90.94 | | C |
| ANISOU | 1891 | CA | ARG | A | 262 | 12311 | 9982 | 12260 | 3291 | 5065 | 868 | C |
| ATOM | 1892 | C | ARG | A | 262 | 82.052 | −45.171 | 105.240 | 1.00 | 96.95 | | C |
| ANISOU | 1892 | C | ARG | A | 262 | 13004 | 10936 | 12897 | 3256 | 4939 | 752 | C |
| ATOM | 1893 | O | ARG | A | 262 | 80.914 | −44.749 | 105.016 | 1.00 | 96.91 | | O |
| ANISOU | 1893 | O | ARG | A | 262 | 12964 | 11148 | 12708 | 3734 | 5118 | 1024 | O |
| ATOM | 1894 | CB | ARG | A | 262 | 83.324 | −46.417 | 103.470 | 1.00 | 89.85 | | C |
| ANISOU | 1894 | CB | ARG | A | 262 | 11862 | 10348 | 11929 | 3171 | 4512 | 843 | C |
| ATOM | 1895 | CG | ARG | A | 262 | 82.107 | −46.967 | 102.754 | 1.00 | 97.30 | | C |
| ANISOU | 1895 | CG | ARG | A | 262 | 12506 | 11961 | 12503 | 3581 | 4302 | 1071 | C |
| ATOM | 1896 | N | GLU | A | 263 | 82.405 | −45.781 | 106.394 | 1.00 | 94.58 | | N |
| ANISOU | 1896 | N | GLU | A | 263 | 12661 | 10618 | 12658 | 2737 | 4631 | 370 | N |
| ATOM | 1897 | CA | GLU | A | 263 | 81.465 | −46.006 | 107.500 | 1.00 | 94.63 | | C |
| ANISOU | 1897 | CA | GLU | A | 263 | 12602 | 10810 | 12541 | 2661 | 4490 | 239 | C |
| ATOM | 1898 | CB | GLU | A | 263 | 81.693 | −47.379 | 108.189 | 1.00 | 95.85 | | C |
| ANISOU | 1898 | CB | GLU | A | 263 | 12560 | 11263 | 12594 | 2238 | 3955 | −27 | C |
| ATOM | 1899 | CG | GLU | A | 263 | 81.849 | −48.588 | 107.258 | 1.00 | 105.86 | | C |
| ANISOU | 1899 | CG | GLU | A | 263 | 13588 | 12912 | 13724 | 2226 | 3586 | 51 | C |
| ATOM | 1900 | CD | GLU | A | 263 | 80.692 | −49.140 | 106.430 | 1.00 | 123.42 | | C |
| ANISOU | 1900 | CD | GLU | A | 263 | 15545 | 15662 | 15686 | 2543 | 3455 | 242 | C |
| ATOM | 1901 | OE1 | GLU | A | 263 | 80.878 | −50.210 | 105.804 | 1.00 | 112.40 | | O |
| ANISOU | 1901 | OE1 | GLU | A | 263 | 13946 | 14561 | 14201 | 2430 | 3172 | 205 | O |
| ATOM | 1902 | OE2 | GLU | A | 263 | 79.608 | −48.516 | 106.397 | 1.00 | 117.45 | | O |
| ANISOU | 1902 | OE2 | GLU | A | 263 | 14760 | 15062 | 14805 | 2901 | 3652 | 400 | O |
| ATOM | 1903 | C | GLU | A | 263 | 81.442 | −44.851 | 108.524 | 1.00 | 98.61 | | C |
| ANISOU | 1903 | C | GLU | A | 263 | 13376 | 10849 | 13243 | 2575 | 4921 | 79 | C |
| ATOM | 1904 | O | GLU | A | 263 | 80.363 | −44.502 | 109.010 | 1.00 | 98.23 | | O |
| ANISOU | 1904 | O | GLU | A | 263 | 13344 | 10884 | 13096 | 2793 | 5049 | 160 | O |
| ATOM | 1905 | N | GLY | A | 264 | 82.610 | −44.277 | 108.825 | 1.00 | 94.76 | | N |
| ANISOU | 1905 | N | GLY | A | 264 | 13071 | 9906 | 13027 | 2245 | 5162 | −180 | N |
| ATOM | 1906 | CA | GLY | A | 264 | 82.753 | −43.179 | 109.777 | 1.00 | 94.01 | | C |
| ANISOU | 1906 | CA | GLY | A | 264 | 13215 | 9353 | 13153 | 2063 | 5624 | −444 | C |
| ATOM | 1907 | C | GLY | A | 264 | 83.402 | −43.526 | 111.110 | 1.00 | 96.63 | | C |
| ANISOU | 1907 | C | GLY | A | 264 | 13475 | 9738 | 13502 | 1503 | 5377 | −957 | C |
| ATOM | 1908 | O | GLY | A | 264 | 83.592 | −42.630 | 111.942 | 1.00 | 97.47 | | O |
| ANISOU | 1908 | O | GLY | A | 264 | 13738 | 9516 | 13779 | 1284 | 5758 | −1271 | O |
| ATOM | 1909 | N | LYS | A | 265 | 83.737 | −44.817 | 111.341 | 1.00 | 90.78 | | N |
| ANISOU | 1909 | N | LYS | A | 265 | 12494 | 9432 | 12567 | 1288 | 4782 | −1051 | N |
| ATOM | 1910 | CA | LYS | A | 265 | 84.353 | −45.335 | 112.574 | 1.00 | 89.45 | | C |
| ANISOU | 1910 | CA | LYS | A | 265 | 12213 | 9460 | 12314 | 861 | 4487 | −1464 | C |
| ATOM | 1911 | CB | LYS | A | 265 | 84.230 | −46.874 | 112.596 | 1.00 | 90.74 | | C |
| ANISOU | 1911 | CB | LYS | A | 265 | 12154 | 10106 | 12216 | 859 | 3904 | −1340 | C |
| ATOM | 1912 | C | LYS | A | 265 | 85.836 | −44.896 | 112.758 | 1.00 | 92.70 | | C |
| ANISOU | 1912 | C | LYS | A | 265 | 12626 | 9683 | 12912 | 461 | 4632 | −1877 | C |
| ATOM | 1913 | O | LYS | A | 265 | 86.541 | −44.752 | 111.761 | 1.00 | 93.26 | | O |
| ANISOU | 1913 | O | LYS | A | 265 | 12724 | 9571 | 13140 | 479 | 4751 | −1790 | O |
| ATOM | 1914 | N | THR | A | 266 | 86.308 | −44.698 | 114.027 | 1.00 | 87.70 | | N |
| ANISOU | 1914 | N | THR | A | 266 | 11931 | 9155 | 12237 | 100 | 4623 | −2355 | N |
| ATOM | 1915 | CA | THR | A | 266 | 87.716 | −44.389 | 114.362 | 1.00 | 87.17 | | C |
| ANISOU | 1915 | CA | THR | A | 266 | 11760 | 9090 | 12273 | −329 | 4703 | −2859 | C |
| ATOM | 1916 | CB | THR | A | 266 | 87.792 | −43.828 | 115.781 | 1.00 | 86.15 | | C |
| ANISOU | 1916 | CB | THR | A | 266 | 11582 | 9067 | 12083 | −645 | 4845 | −3389 | C |
| ATOM | 1917 | C | THR | A | 266 | 88.534 | −45.723 | 114.243 | 1.00 | 93.77 | | C |
| ANISOU | 1917 | C | THR | A | 266 | 12338 | 10410 | 12882 | −389 | 4120 | −2822 | C |
| ATOM | 1918 | O | THR | A | 266 | 87.902 | −46.765 | 114.029 | 1.00 | 94.42 | | O |
| ANISOU | 1918 | O | THR | A | 266 | 12376 | 10733 | 12768 | −133 | 3750 | −2444 | O |
| ATOM | 1919 | N | LEU | A | 267 | 89.906 | −45.726 | 114.371 | 1.00 | 90.07 | | N |
| ANISOU | 1919 | N | LEU | A | 267 | 11691 | 10097 | 12436 | −715 | 4069 | −3219 | N |
| ATOM | 1920 | CA | LEU | A | 267 | 90.688 | −47.000 | 114.306 | 1.00 | 89.29 | | C |
| ANISOU | 1920 | CA | LEU | A | 267 | 11351 | 10476 | 12097 | −710 | 3546 | −3152 | C |
| ATOM | 1921 | CB | LEU | A | 267 | 92.205 | −46.778 | 114.150 | 1.00 | 88.86 | | C |
| ANISOU | 1921 | CB | LEU | A | 267 | 11103 | 10531 | 12128 | −1032 | 3591 | −3567 | C |
| ATOM | 1922 | C | LEU | A | 267 | 90.387 | −47.867 | 115.550 | 1.00 | 92.69 | | C |
| ANISOU | 1922 | C | LEU | A | 267 | 11635 | 11440 | 12141 | −657 | 3159 | −3189 | C |
| ATOM | 1923 | O | LEU | A | 267 | 90.342 | −49.099 | 115.471 | 1.00 | 90.50 | | O |
| ANISOU | 1923 | O | LEU | A | 267 | 11282 | 11454 | 11649 | −459 | 2776 | −2883 | O |
| ATOM | 1924 | N | GLU | A | 268 | 90.116 | −47.179 | 116.686 | 1.00 | 90.80 | | N |
| ANISOU | 1924 | N | GLU | A | 268 | 11387 | 11285 | 11828 | −820 | 3328 | −3554 | N |
| ATOM | 1925 | CA | GLU | A | 268 | 89.705 | −47.728 | 117.983 | 1.00 | 90.71 | | C |
| ANISOU | 1925 | CA | GLU | A | 268 | 11274 | 11741 | 11452 | −759 | 3072 | −3619 | C |
| ATOM | 1926 | CB | GLU | A | 268 | 89.660 | −46.598 | 119.034 | 1.00 | 92.02 | | C |
| ANISOU | 1926 | CB | GLU | A | 268 | 11417 | 11921 | 11627 | −1043 | 3386 | −4168 | C |
| ATOM | 1927 | C | GLU | A | 268 | 88.315 | −48.411 | 117.834 | 1.00 | 92.85 | | C |
| ANISOU | 1927 | C | GLU | A | 268 | 11723 | 11896 | 11658 | −414 | 2949 | −3082 | C |
| ATOM | 1928 | O | GLU | A | 268 | 88.036 | −49.409 | 118.505 | 1.00 | 91.40 | | O |
| ANISOU | 1928 | O | GLU | A | 268 | 11475 | 12081 | 11173 | −260 | 2654 | −2921 | O |
| ATOM | 1929 | N | GLN | A | 269 | 87.482 | −47.867 | 116.907 | 1.00 | 88.16 | | N |
| ANISOU | 1929 | N | GLN | A | 269 | 11341 | 10814 | 11341 | −277 | 3216 | −2813 | N |
| ATOM | 1930 | CA | GLN | A | 269 | 86.149 | −48.314 | 116.500 | 1.00 | 86.67 | | C |
| ANISOU | 1930 | CA | GLN | A | 269 | 11276 | 10517 | 11138 | 28 | 3171 | −2362 | C |
| ATOM | 1931 | CB | GLN | A | 269 | 85.253 | −47.087 | 116.213 | 1.00 | 87.68 | | C |
| ANISOU | 1931 | CB | GLN | A | 269 | 11604 | 10215 | 11495 | 126 | 3615 | −2317 | C |
| ATOM | 1932 | C | GLN | A | 269 | 86.186 | −49.344 | 115.302 | 1.00 | 88.10 | | C |
| ANISOU | 1932 | C | GLN | A | 269 | 11417 | 10727 | 11332 | 212 | 2937 | −1970 | C |
| ATOM | 1933 | O | GLN | A | 269 | 85.190 | −49.502 | 114.583 | 1.00 | 87.97 | | O |
| ANISOU | 1933 | O | GLN | A | 269 | 11463 | 10600 | 11361 | 438 | 2976 | −1653 | O |
| ATOM | 1934 | N | ILE | A | 270 | 87.327 | −50.052 | 115.114 | 1.00 | 81.95 | | N |
| ANISOU | 1934 | N | ILE | A | 270 | 10503 | 10149 | 10487 | 116 | 2700 | −2024 | N |
| ATOM | 1935 | CA | ILE | A | 270 | 87.454 | −51.115 | 114.111 | 1.00 | 80.93 | | C |
| ANISOU | 1935 | CA | ILE | A | 270 | 10328 | 10067 | 10356 | 252 | 2488 | −1708 | C |
| ATOM | 1936 | CB | ILE | A | 270 | 88.461 | −50.830 | 112.948 | 1.00 | 83.63 | | C |
| ANISOU | 1936 | CB | ILE | A | 270 | 10643 | 10230 | 10904 | 186 | 2549 | −1719 | C |
| ATOM | 1937 | CG1 | ILE | A | 270 | 88.233 | −49.440 | 112.300 | 1.00 | 83.36 | | C |
| ANISOU | 1937 | CG1 | ILE | A | 270 | 10755 | 9779 | 11141 | 194 | 2959 | −1753 | C |
| ATOM | 1938 | CD1 | ILE | A | 270 | 87.665 | −49.391 | 110.820 | 1.00 | 83.32 | | C |
| ANISOU | 1938 | CD1 | ILE | A | 270 | 10825 | 9571 | 11263 | 463 | 3070 | −1375 | C |
| ATOM | 1939 | CG2 | ILE | A | 270 | 88.443 | −51.978 | 111.905 | 1.00 | 83.89 | | C |
| ANISOU | 1939 | CG2 | ILE | A | 270 | 10628 | 10326 | 10920 | 333 | 2340 | −1395 | C |
| ATOM | 1940 | C | ILE | A | 270 | 87.750 | −52.456 | 114.817 | 1.00 | 83.80 | | C |
| ANISOU | 1940 | C | ILE | A | 270 | 10591 | 10802 | 10448 | 291 | 2182 | −1641 | C |
| ATOM | 1941 | O | ILE | A | 270 | 88.864 | −52.656 | 115.328 | 1.00 | 83.65 | | O |
| ANISOU | 1941 | O | ILE | A | 270 | 10447 | 11038 | 10297 | 198 | 2052 | −1833 | O |
| ATOM | 1942 | O | TYR | A | 271 | 88.394 | −56.538 | 115.038 | 1.00 | 76.35 | | O |
| ANISOU | 1942 | O | TYR | A | 271 | 9571 | 10384 | 9054 | 648 | 1708 | −1014 | O |
| ATOM | 1943 | N | TYR | A | 271 | 86.750 | −53.372 | 114.816 | 1.00 | 78.54 | | N |
| ANISOU | 1943 | N | TYR | A | 271 | 9970 | 10179 | 9694 | 447 | 2113 | −1371 | N |
| ATOM | 1944 | CA | TYR | A | 271 | 86.806 | −54.729 | 115.393 | 1.00 | 77.08 | | C |
| ANISOU | 1944 | CA | TYR | A | 271 | 9762 | 10236 | 9291 | 538 | 1947 | −1222 | C |
| ATOM | 1945 | C | TYR | A | 271 | 87.631 | −55.706 | 114.522 | 1.00 | 77.70 | | C |
| ANISOU | 1945 | C | TYR | A | 271 | 9785 | 10331 | 9407 | 573 | 1823 | −1063 | C |
| ATOM | 1946 | CB | TYR | A | 271 | 85.383 | −55.259 | 115.557 | 1.00 | 78.21 | | C |
| ANISOU | 1946 | CB | TYR | A | 271 | 9982 | 10331 | 9402 | 634 | 2026 | −1042 | C |
| ATOM | 1947 | N | ASP | A | 272 | 87.472 | −55.552 | 113.185 | 1.00 | 72.55 | | N |
| ANISOU | 1947 | N | ASP | A | 272 | 9129 | 9464 | 8972 | 557 | 1869 | −973 | N |
| ATOM | 1948 | CA | ASP | A | 272 | 88.084 | −56.278 | 112.066 | 1.00 | 70.64 | | C |
| ANISOU | 1948 | CA | ASP | A | 272 | 8838 | 9176 | 8826 | 567 | 1795 | −841 | C |
| ATOM | 1949 | C | ASP | A | 272 | 89.604 | −56.315 | 112.074 | 1.00 | 74.73 | | C |
| ANISOU | 1949 | C | ASP | A | 272 | 9274 | 9803 | 9318 | 517 | 1677 | −942 | C |
| ATOM | 1950 | O | ASP | A | 272 | 90.167 | −57.093 | 111.298 | 1.00 | 75.61 | | O |
| ANISOU | 1950 | O | ASP | A | 272 | 9350 | 9900 | 9477 | 546 | 1610 | −815 | O |
| ATOM | 1951 | CB | ASP | A | 272 | 87.653 | −55.624 | 110.734 | 1.00 | 71.19 | | C |
| ANISOU | 1951 | CB | ASP | A | 272 | 8898 | 9055 | 9094 | 577 | 1894 | −790 | C |
| ATOM | 1952 | CG | ASP | A | 272 | 86.472 | −56.234 | 110.009 | 1.00 | 75.22 | | C |
| ANISOU | 1952 | CG | ASP | A | 272 | 9377 | 9587 | 9616 | 654 | 1939 | −640 | C |
| ATOM | 1953 | OD1 | ASP | A | 272 | 86.484 | −57.456 | 109.769 | 1.00 | 73.60 | | O |
| ANISOU | 1953 | OD1 | ASP | A | 272 | 9139 | 9442 | 9383 | 632 | 1891 | −562 | O |
| ATOM | 1954 | OD2 | ASP | A | 272 | 85.556 | −55.481 | 109.646 | 1.00 | 84.27 | | O |
| ANISOU | 1954 | OD2 | ASP | A | 272 | 10514 | 10710 | 10795 | 740 | 2054 | −620 | O |
| ATOM | 1955 | N | LEU | A | 273 | 90.285 | −55.480 | 112.892 | 1.00 | 70.31 | | N |
| ANISOU | 1955 | N | LEU | A | 273 | 8653 | 9380 | 8681 | 425 | 1668 | −1206 | N |
| ATOM | 1956 | CA | LEU | A | 273 | 91.752 | −55.388 | 112.895 | 1.00 | 69.94 | | C |
| ANISOU | 1956 | CA | LEU | A | 273 | 8458 | 9522 | 8596 | 346 | 1567 | −1387 | C |
| ATOM | 1957 | C | LEU | A | 273 | 92.468 | −56.725 | 113.103 | 1.00 | 74.45 | | C |
| ANISOU | 1957 | C | LEU | A | 273 | 8958 | 10368 | 8962 | 516 | 1395 | −1207 | C |
| ATOM | 1958 | O | LEU | A | 273 | 93.646 | −56.838 | 112.769 | 1.00 | 75.15 | | O |
| ANISOU | 1958 | O | LEU | A | 273 | 8911 | 10601 | 9041 | 493 | 1304 | −1284 | O |
| ATOM | 1959 | CB | LEU | A | 273 | 92.262 | −54.321 | 113.848 | 1.00 | 69.79 | | C |
| ANISOU | 1959 | CB | LEU | A | 273 | 8332 | 9692 | 8494 | 178 | 1617 | −1791 | C |
| ATOM | 1960 | CG | LEU | A | 273 | 91.829 | −52.903 | 113.464 | 1.00 | 74.57 | | C |
| ANISOU | 1960 | CG | LEU | A | 273 | 9032 | 9928 | 9373 | 0 | 1892 | −1982 | C |
| ATOM | 1961 | CD2 | LEU | A | 273 | 92.148 | −52.589 | 112.028 | 1.00 | 76.67 | | C |
| ANISOU | 1961 | CD2 | LEU | A | 273 | 9348 | 9858 | 9927 | −34 | 2010 | −1881 | C |
| ATOM | 1962 | CD1 | LEU | A | 273 | 92.499 | −51.888 | 114.321 | 1.00 | 75.27 | | C |
| ANISOU | 1962 | CD1 | LEU | A | 273 | 8996 | 10180 | 9424 | −243 | 2007 | −2469 | C |
| ATOM | 1963 | N | LEU | A | 274 | 91.733 | −57.748 | 113.554 | 1.00 | 69.57 | | N |
| ANISOU | 1963 | N | LEU | A | 274 | 8447 | 9778 | 8207 | 697 | 1406 | −950 | N |
| ATOM | 1964 | CA | LEU | A | 274 | 92.155 | −59.137 | 113.654 | 1.00 | 68.08 | | C |
| ANISOU | 1964 | CA | LEU | A | 274 | 8280 | 9718 | 7871 | 916 | 1370 | −684 | C |
| ATOM | 1965 | C | LEU | A | 274 | 90.863 | −59.937 | 113.592 | 1.00 | 71.09 | | C |
| ANISOU | 1965 | C | LEU | A | 274 | 8846 | 9852 | 8314 | 975 | 1536 | −449 | C |
| ATOM | 1966 | O | LEU | A | 274 | 90.047 | −59.841 | 114.504 | 1.00 | 71.02 | | O |
| ANISOU | 1966 | O | LEU | A | 274 | 8909 | 9893 | 8185 | 1016 | 1607 | −450 | O |
| ATOM | 1967 | CB | LEU | A | 274 | 92.945 | −59.421 | 114.939 | 1.00 | 67.77 | | C |
| ANISOU | 1967 | CB | LEU | A | 274 | 8128 | 10171 | 7451 | 1124 | 1272 | −713 | C |
| ATOM | 1968 | N | ALA | A | 275 | 90.600 | −60.594 | 112.463 | 1.00 | 67.43 | | N |
| ANISOU | 1968 | N | ALA | A | 275 | 8433 | 9128 | 8058 | 928 | 1618 | −313 | N |
| ATOM | 1969 | CA | ALA | A | 275 | 89.386 | −61.396 | 112.295 | 1.00 | 67.75 | | C |
| ANISOU | 1969 | CA | ALA | A | 275 | 8597 | 8964 | 8180 | 914 | 1819 | −182 | C |
| ATOM | 1970 | C | ALA | A | 275 | 89.729 | −62.841 | 111.961 | 1.00 | 76.15 | | C |
| ANISOU | 1970 | C | ALA | A | 275 | 9747 | 9915 | 9273 | 1013 | 1975 | 29 | C |
| ATOM | 1971 | O | ALA | A | 275 | 90.781 | −63.116 | 111.352 | 1.00 | 77.31 | | O |
| ANISOU | 1971 | O | ALA | A | 275 | 9840 | 10078 | 9458 | 1055 | 1898 | 76 | O |
| ATOM | 1972 | CB | ALA | A | 275 | 88.479 | −60.797 | 111.240 | 1.00 | 68.16 | | C |
| ANISOU | 1972 | CB | ALA | A | 275 | 8596 | 8858 | 8444 | 735 | 1844 | −293 | C |
| ATOM | 1973 | N | VAL | A | 276 | 88.861 | −63.770 | 112.400 | 1.00 | 73.15 | | N |
| ANISOU | 1973 | N | VAL | A | 276 | 9513 | 9397 | 8885 | 1051 | 2245 | 151 | N |
| ATOM | 1974 | CA | VAL | A | 276 | 89.048 | −65.210 | 112.235 | 1.00 | 73.44 | | C |
| ANISOU | 1974 | CA | VAL | A | 276 | 9693 | 9238 | 8974 | 1147 | 2535 | 355 | C |
| ATOM | 1975 | C | VAL | A | 276 | 87.718 | −65.875 | 111.801 | 1.00 | 80.71 | | C |
| ANISOU | 1975 | C | VAL | A | 276 | 10677 | 9893 | 10096 | 921 | 2860 | 270 | C |
| ATOM | 1976 | O | VAL | A | 276 | 86.678 | −65.579 | 112.391 | 1.00 | 80.82 | | O |
| ANISOU | 1976 | O | VAL | A | 276 | 10709 | 9927 | 10074 | 855 | 2933 | 191 | O |
| ATOM | 1977 | CB | VAL | A | 276 | 89.643 | −65.799 | 113.549 | 1.00 | 76.47 | | C |
| ANISOU | 1977 | CB | VAL | A | 276 | 10214 | 9770 | 9070 | 1517 | 2643 | 625 | C |
| ATOM | 1978 | CG1 | VAL | A | 276 | 89.370 | −67.288 | 113.674 | 1.00 | 76.36 | | C |
| ANISOU | 1978 | CG1 | VAL | A | 276 | 10444 | 9448 | 9123 | 1646 | 3121 | 876 | C |
| ATOM | 1979 | CG2 | VAL | A | 276 | 91.142 | −65.527 | 113.660 | 1.00 | 75.80 | | C |
| ANISOU | 1979 | CG2 | VAL | A | 276 | 10001 | 10005 | 8794 | 1736 | 2375 | 683 | C |
| ATOM | 1980 | N | ARG | A | 277 | 87.758 | −66.751 | 110.764 | 1.00 | 78.61 | | N |
| ANISOU | 1980 | N | ARG | A | 277 | 10416 | 9414 | 10038 | 779 | 3065 | 238 | N |
| ATOM | 1981 | CA | ARG | A | 277 | 86.596 | −67.480 | 110.232 | 1.00 | 79.02 | | C |
| ANISOU | 1981 | CA | ARG | A | 277 | 10466 | 9269 | 10288 | 501 | 3414 | 57 | C |
| ATOM | 1982 | C | ARG | A | 277 | 86.664 | −68.933 | 110.606 | 1.00 | 86.38 | | C |
| ANISOU | 1982 | C | ARG | A | 277 | 11651 | 9868 | 11302 | 570 | 3932 | 227 | C |
| ATOM | 1983 | O | ARG | A | 277 | 87.735 | −69.529 | 110.562 | 1.00 | 85.86 | | O |
| ANISOU | 1983 | O | ARG | A | 277 | 11706 | 9695 | 11221 | 783 | 4009 | 457 | O |
| ATOM | 1984 | CB | ARG | A | 277 | 86.513 | −67.389 | 108.695 | 1.00 | 78.78 | | C |
| ANISOU | 1984 | CB | ARG | A | 277 | 10217 | 9286 | 10430 | 245 | 3322 | −189 | C |
| ATOM | 1985 | CG | ARG | A | 277 | 85.820 | −66.150 | 108.148 | 1.00 | 89.35 | | C |
| ANISOU | 1985 | CG | ARG | A | 277 | 11307 | 10912 | 11731 | 133 | 3014 | −401 | C |
| ATOM | 1986 | CD | ARG | A | 277 | 84.744 | −66.480 | 107.132 | 1.00 | 97.36 | | C |
| ANISOU | 1986 | CD | ARG | A | 277 | 12104 | 12033 | 12855 | −154 | 3175 | −715 | C |
| ATOM | 1987 | NE | ARG | A | 277 | 85.206 | −66.346 | 105.748 | 1.00 | 107.70 | | N |
| ANISOU | 1987 | NE | ARG | A | 277 | 13227 | 13476 | 14219 | −222 | 3020 | −821 | N |
| ATOM | 1988 | CZ | ARG | A | 277 | 85.109 | −65.239 | 105.016 | 1.00 | 120.57 | | C |
| ANISOU | 1988 | CZ | ARG | A | 277 | 14655 | 15390 | 15767 | −157 | 2720 | −872 | C |
| ATOM | 1989 | NH1 | ARG | A | 277 | 84.594 | −64.132 | 105.536 | 1.00 | 101.76 | | N |
| ANISOU | 1989 | NH1 | ARG | A | 277 | 12242 | 13157 | 13267 | −28 | 2554 | −831 | N |
| ATOM | 1990 | NH2 | ARG | A | 277 | 85.545 | −65.225 | 103.762 | 1.00 | 110.33 | | N |
| ANISOU | 1990 | NH2 | ARG | A | 277 | 13204 | 14213 | 14503 | −193 | 2626 | −944 | N |
| ATOM | 1991 | N | VAL | A | 278 | 85.511 | −69.514 | 110.936 | 1.00 | 87.36 | | N |
| ANISOU | 1991 | N | VAL | A | 278 | 11855 | 9814 | 11525 | 391 | 4338 | 109 | N |
| ATOM | 1992 | CA | VAL | A | 278 | 85.354 | −70.934 | 111.255 | 1.00 | 88.85 | | C |
| ANISOU | 1992 | CA | VAL | A | 278 | 12319 | 9584 | 11857 | 396 | 4991 | 227 | C |
| ATOM | 1993 | C | VAL | A | 278 | 84.352 | −71.511 | 110.242 | 1.00 | 100.36 | | C |
| ANISOU | 1993 | C | VAL | A | 278 | 13628 | 10902 | 13602 | −101 | 5332 | −214 | C |
| ATOM | 1994 | O | VAL | A | 278 | 83.152 | −71.214 | 110.314 | 1.00 | 99.20 | | O |
| ANISOU | 1994 | O | VAL | A | 278 | 13327 | 10886 | 13479 | −359 | 5376 | −503 | O |
| ATOM | 1995 | CB | VAL | A | 278 | 84.996 | −71.225 | 112.740 | 1.00 | 90.84 | | C |
| ANISOU | 1995 | CB | VAL | A | 278 | 12839 | 9721 | 11955 | 662 | 5289 | 499 | C |
| ATOM | 1996 | CG1 | VAL | A | 278 | 84.636 | −72.691 | 112.937 | 1.00 | 90.28 | | C |
| ANISOU | 1996 | CG1 | VAL | A | 278 | 13071 | 9133 | 12098 | 613 | 6096 | 581 | C |
| ATOM | 1997 | CG2 | VAL | A | 278 | 86.146 | −70.843 | 113.665 | 1.00 | 90.12 | | C |
| ANISOU | 1997 | CG2 | VAL | A | 278 | 12850 | 9860 | 11533 | 1182 | 4993 | 908 | C |
| ATOM | 1998 | N | ILE | A | 279 | 84.882 | −72.261 | 109.247 | 1.00 | 103.33 | | N |
| ANISOU | 1998 | N | ILE | A | 279 | 14000 | 11089 | 14170 | −239 | 5535 | −303 | N |
| ATOM | 1999 | CA | ILE | A | 279 | 84.097 | −72.893 | 108.181 | 1.00 | 105.74 | | C |
| ANISOU | 1999 | CA | ILE | A | 279 | 14121 | 11322 | 14732 | −735 | 5878 | −789 | C |
| ATOM | 2000 | C | ILE | A | 279 | 83.892 | −74.371 | 108.590 | 1.00 | 118.19 | | C |
| ANISOU | 2000 | C | ILE | A | 279 | 16035 | 12317 | 16555 | −833 | 6741 | −759 | C |
| ATOM | 2001 | O | ILE | A | 279 | 84.871 | −75.091 | 108.787 | 1.00 | 118.39 | | O |
| ANISOU | 2001 | O | ILE | A | 279 | 16364 | 11992 | 16627 | −545 | 7018 | −399 | O |
| ATOM | 2002 | CB | ILE | A | 279 | 84.704 | −72.680 | 106.750 | 1.00 | 107.84 | | C |
| ANISOU | 2002 | CB | ILE | A | 279 | 14129 | 11799 | 15046 | −871 | 5561 | −981 | C |
| ATOM | 2003 | CG1 | ILE | A | 279 | 85.233 | −71.246 | 106.547 | 1.00 | 107.55 | | C |
| ANISOU | 2003 | CG1 | ILE | A | 279 | 13895 | 12199 | 14771 | −636 | 4805 | −845 | C |
| ATOM | 2004 | CG2 | ILE | A | 279 | 83.650 | −72.931 | 105.686 | 1.00 | 108.18 | | C |
| ANISOU | 2004 | CG2 | ILE | A | 279 | 13829 | 12046 | 15227 | −1384 | 5739 | −1580 | C |
| ATOM | 2005 | CD1 | ILE | A | 279 | 86.671 | −71.042 | 106.774 | 1.00 | 111.64 | | C |
| ANISOU | 2005 | CD1 | ILE | A | 279 | 14573 | 12655 | 15189 | −258 | 4544 | −443 | C |
| ATOM | 2006 | N | LEU | A | 280 | 82.632 | −74.795 | 108.779 | 1.00 | 120.32 | | N |
| ANISOU | 2006 | N | LEU | A | 280 | 16261 | 12482 | 16974 | −1203 | 7204 | −1115 | N |
| ATOM | 2007 | CA | LEU | A | 280 | 82.331 | −76.137 | 109.270 | 1.00 | 123.41 | | C |
| ANISOU | 2007 | CA | LEU | A | 280 | 17006 | 12256 | 17628 | −1315 | 8128 | −1100 | C |
| ATOM | 2008 | C | LEU | A | 280 | 81.530 | −77.003 | 108.287 | 1.00 | 136.62 | | C |
| ANISOU | 2008 | C | LEU | A | 280 | 18491 | 13779 | 19641 | −1973 | 8713 | −1767 | C |
| ATOM | 2009 | O | LEU | A | 280 | 80.481 | −76.562 | 107.823 | 1.00 | 136.20 | | O |
| ANISOU | 2009 | O | LEU | A | 280 | 18022 | 14158 | 19571 | −2384 | 8541 | −2302 | O |
| ATOM | 2010 | CB | LEU | A | 280 | 81.514 | −76.011 | 110.583 | 1.00 | 123.45 | | C |
| ANISOU | 2010 | CB | LEU | A | 280 | 17183 | 12182 | 17538 | −1197 | 8337 | −950 | C |
| ATOM | 2011 | CG | LEU | A | 280 | 82.034 | −75.109 | 111.708 | 1.00 | 127.44 | | C |
| ANISOU | 2011 | CG | LEU | A | 280 | 17820 | 12923 | 17676 | −619 | 7804 | −407 | C |
| ATOM | 2012 | CD1 | LEU | A | 280 | 80.894 | −74.349 | 112.363 | 1.00 | 127.20 | | C |
| ANISOU | 2012 | CD1 | LEU | A | 280 | 17630 | 13183 | 17518 | −736 | 7612 | −581 | C |
| ATOM | 2013 | CD2 | LEU | A | 280 | 82.762 | −75.915 | 112.749 | 1.00 | 129.63 | | C |
| ANISOU | 2013 | CD2 | LEU | A | 280 | 18596 | 12761 | 17896 | −108 | 8294 | 187 | C |
| ATOM | 2014 | N | ASP | A | 281 | 81.968 | −78.255 | 108.010 | 1.00 | 140.77 | | N |
| ANISOU | 2014 | N | ASP | A | 281 | 19305 | 13714 | 20467 | −2076 | 9461 | −1769 | N |
| ATOM | 2015 | CA | ASP | A | 281 | 81.117 | −79.148 | 107.206 | 1.00 | 144.07 | | C |
| ANISOU | 2015 | CA | ASP | A | 281 | 19549 | 13954 | 21235 | −2774 | 10149 | −2493 | C |
| ATOM | 2016 | C | ASP | A | 281 | 80.539 | −80.253 | 108.096 | 1.00 | 155.37 | | C |
| ANISOU | 2016 | C | ASP | A | 281 | 21403 | 14670 | 22960 | −2900 | 11213 | −2477 | C |
| ATOM | 2017 | O | ASP | A | 281 | 81.287 | −81.034 | 108.685 | 1.00 | 155.42 | | O |
| ANISOU | 2017 | O | ASP | A | 281 | 21941 | 14034 | 23079 | −2509 | 11775 | −1944 | O |
| ATOM | 2018 | CB | ASP | A | 281 | 81.711 | −79.688 | 105.880 | 1.00 | 145.94 | | C |
| ANISOU | 2018 | CB | ASP | A | 281 | 19647 | 14147 | 21659 | −3049 | 10268 | −2812 | C |
| ATOM | 2019 | CG | ASP | A | 281 | 83.143 | −80.169 | 105.843 | 1.00 | 158.22 | | C |
| ANISOU | 2019 | CG | ASP | A | 281 | 21593 | 15265 | 23258 | −2591 | 10373 | −2247 | C |
| ATOM | 2020 | OD1 | ASP | A | 281 | 83.850 | −80.015 | 106.858 | 1.00 | 158.54 | | O |
| ANISOU | 2020 | OD1 | ASP | A | 281 | 22001 | 15114 | 23123 | −1962 | 10261 | −1534 | O |
| ATOM | 2021 | OD2 | ASP | A | 281 | 83.564 | −80.685 | 104.784 | 1.00 | 167.99 | | O |
| ANISOU | 2021 | OD2 | ASP | A | 281 | 22736 | 16416 | 24676 | −2847 | 10552 | −2536 | O |
| ATOM | 2022 | N | PRO | A | 282 | 79.206 | −80.267 | 108.268 | 1.00 | 156.88 | | N |
| ANISOU | 2022 | N | PRO | A | 282 | 21366 | 14997 | 23245 | −3384 | 11498 | −3013 | N |
| ATOM | 2023 | CA | PRO | A | 282 | 78.590 | −81.271 | 109.156 | 1.00 | 158.36 | | C |
| ANISOU | 2023 | CA | PRO | A | 282 | 21965 | 14478 | 23728 | −3526 | 12564 | −3013 | C |
| ATOM | 2024 | C | PRO | A | 282 | 78.582 | −82.714 | 108.628 | 1.00 | 165.84 | | C |
| ANISOU | 2024 | C | PRO | A | 282 | 22964 | 15146 | 24900 | −3776 | 12979 | −3170 | C |
| ATOM | 2025 | O | PRO | A | 282 | 78.779 | −82.942 | 107.431 | 1.00 | 165.37 | | O |
| ANISOU | 2025 | O | PRO | A | 282 | 22609 | 15288 | 24935 | −4120 | 12866 | −3606 | O |
| ATOM | 2026 | CB | PRO | A | 282 | 77.169 | −80.734 | 109.348 | 1.00 | 160.17 | | C |
| ANISOU | 2026 | CB | PRO | A | 282 | 21778 | 15189 | 23890 | −3957 | 12429 | −3560 | C |
| ATOM | 2027 | CG | PRO | A | 282 | 76.895 | −79.969 | 108.099 | 1.00 | 164.61 | | C |
| ANISOU | 2027 | CG | PRO | A | 282 | 21656 | 16604 | 24284 | −4300 | 11667 | −4142 | C |
| ATOM | 2028 | CD | PRO | A | 282 | 78.197 | −79.359 | 107.682 | 1.00 | 159.58 | | C |
| ANISOU | 2028 | CD | PRO | A | 282 | 21062 | 16163 | 23409 | −3781 | 10894 | −3621 | C |
| ATOM | 2029 | N | LYS | A | 283 | 78.338 | −83.687 | 109.534 | 1.00 | 164.80 | | N |
| ANISOU | 2029 | N | LYS | A | 283 | 23136 | 14735 | 24745 | −3525 | 13193 | −2744 | N |
| ATOM | 2030 | CA | LYS | A | 283 | 78.212 | −85.107 | 109.200 | 1.00 | 166.00 | | C |
| ANISOU | 2030 | CA | LYS | A | 283 | 23223 | 14944 | 24905 | −3597 | 13051 | −2703 | C |
| ATOM | 2031 | C | LYS | A | 283 | 76.903 | −85.300 | 108.407 | 1.00 | 174.91 | | C |
| ANISOU | 2031 | C | LYS | A | 283 | 23597 | 17090 | 25772 | −3986 | 12048 | −3178 | C |
| ATOM | 2032 | O | LYS | A | 283 | 75.977 | −84.508 | 108.597 | 1.00 | 174.72 | | O |
| ANISOU | 2032 | O | LYS | A | 283 | 23308 | 17377 | 25699 | −4220 | 11994 | −3522 | O |
| ATOM | 2033 | CB | LYS | A | 283 | 78.181 | −85.967 | 110.475 | 1.00 | 167.85 | | C |
| ANISOU | 2033 | CB | LYS | A | 283 | 23797 | 15000 | 24978 | −3120 | 13022 | −2031 | C |
| ATOM | 2034 | CG | LYS | A | 283 | 79.459 | −86.754 | 110.705 | 1.00 | 172.59 | | C |
| ANISOU | 2034 | CG | LYS | A | 283 | 24668 | 15526 | 25381 | −2514 | 12643 | −1315 | C |
| ATOM | 2035 | CD | LYS | A | 283 | 79.285 | −87.811 | 111.785 | 1.00 | 177.59 | | C |
| ANISOU | 2035 | CD | LYS | A | 283 | 25385 | 16343 | 25748 | −2161 | 12227 | −795 | C |
| ATOM | 2036 | CE | LYS | A | 283 | 80.599 | −88.410 | 112.227 | 1.00 | 185.32 | | C |
| ANISOU | 2036 | CE | LYS | A | 283 | 26480 | 17555 | 26378 | −1534 | 11501 | −104 | C |
| ATOM | 2037 | NZ | LYS | A | 283 | 81.383 | −87.481 | 113.084 | 1.00 | 192.46 | | N |
| ANISOU | 2037 | NZ | LYS | A | 283 | 27410 | 18833 | 26883 | −974 | 10836 | 391 | N |
| ATOM | 2038 | N | PRO | A | 284 | 76.788 | −86.313 | 107.515 | 1.00 | 175.19 | | N |
| ANISOU | 2038 | N | PRO | A | 284 | 23437 | 17270 | 25857 | −4208 | 11915 | −3381 | N |
| ATOM | 2039 | CA | PRO | A | 284 | 75.536 | −86.478 | 106.750 | 1.00 | 176.39 | | C |
| ANISOU | 2039 | CA | PRO | A | 284 | 23042 | 18032 | 25946 | −4649 | 11589 | −3933 | C |
| ATOM | 2040 | C | PRO | A | 284 | 74.332 | −86.861 | 107.608 | 1.00 | 183.50 | | C |
| ANISOU | 2040 | C | PRO | A | 284 | 23785 | 19370 | 26568 | −4448 | 11010 | −3619 | C |
| ATOM | 2041 | O | PRO | A | 284 | 74.479 | −87.596 | 108.589 | 1.00 | 182.78 | | O |
| ANISOU | 2041 | O | PRO | A | 284 | 24050 | 18894 | 26505 | −4207 | 11241 | −3190 | O |
| ATOM | 2042 | CB | PRO | A | 284 | 75.880 | −87.575 | 105.734 | 1.00 | 178.06 | | C |
| ANISOU | 2042 | CB | PRO | A | 284 | 23160 | 18326 | 26168 | −4699 | 11360 | −3944 | C |
| ATOM | 2043 | CG | PRO | A | 284 | 77.377 | −87.654 | 105.725 | 1.00 | 181.96 | | C |
| ANISOU | 2043 | CG | PRO | A | 284 | 23982 | 18561 | 26595 | −4236 | 11151 | −3402 | C |
| ATOM | 2044 | CD | PRO | A | 284 | 77.779 | −87.332 | 107.123 | 1.00 | 177.18 | | C |
| ANISOU | 2044 | CD | PRO | A | 284 | 23890 | 17323 | 26108 | −3956 | 11783 | −3018 | C |
| ATOM | 2045 | N | ALA | A | 285 | 73.143 | −86.346 | 107.235 | 1.00 | 183.09 | | N |
| ANISOU | 2045 | N | ALA | A | 285 | 23283 | 19822 | 26460 | −4782 | 10864 | −4111 | N |
| ATOM | 2046 | CA | ALA | A | 285 | 71.890 | −86.586 | 107.951 | 1.00 | 184.38 | | C |
| ANISOU | 2046 | CA | ALA | A | 285 | 23316 | 20206 | 26535 | −4807 | 10766 | −4077 | C |
| ATOM | 2047 | C | ALA | A | 285 | 70.882 | −87.409 | 107.136 | 1.00 | 191.44 | | C |
| ANISOU | 2047 | C | ALA | A | 285 | 23775 | 21760 | 27202 | −4791 | 10001 | −4045 | C |
| ATOM | 2048 | O | ALA | A | 285 | 70.840 | −87.267 | 105.906 | 1.00 | 190.65 | | O |
| ANISOU | 2048 | O | ALA | A | 285 | 23365 | 21973 | 27102 | −5012 | 9856 | −4426 | O |
| ATOM | 2049 | CB | ALA | A | 285 | 71.266 | −85.266 | 108.375 | 1.00 | 185.15 | | C |
| ANISOU | 2049 | CB | ALA | A | 285 | 23211 | 20626 | 26513 | −4841 | 10652 | −4266 | C |
| ATOM | 2050 | N | PRO | A | 286 | 70.045 | −88.251 | 107.812 | 1.00 | 190.79 | | N |
| ANISOU | 2050 | N | PRO | A | 286 | 23738 | 21602 | 27150 | −4817 | 10126 | −3923 | N |
| ATOM | 2051 | CA | PRO | A | 286 | 69.058 | −89.066 | 107.075 | 1.00 | 191.45 | | C |
| ANISOU | 2051 | CA | PRO | A | 286 | 23500 | 22028 | 27214 | −5011 | 9916 | −4117 | C |
| ATOM | 2052 | C | PRO | A | 286 | 68.133 | −88.272 | 106.140 | 1.00 | 196.85 | | C |
| ANISOU | 2052 | C | PRO | A | 286 | 23652 | 23502 | 27638 | −4991 | 9227 | −4299 | C |
| ATOM | 2053 | O | PRO | A | 286 | 68.216 | −88.461 | 104.922 | 1.00 | 196.62 | | O |
| ANISOU | 2053 | O | PRO | A | 286 | 23405 | 23697 | 27604 | −5121 | 9066 | −4531 | O |
| ATOM | 2054 | CB | PRO | A | 286 | 68.290 | −89.807 | 108.191 | 1.00 | 193.19 | | C |
| ANISOU | 2054 | CB | PRO | A | 286 | 23843 | 22165 | 27396 | −4876 | 9913 | −3777 | C |
| ATOM | 2055 | CG | PRO | A | 286 | 68.600 | −89.061 | 109.456 | 1.00 | 197.36 | | C |
| ANISOU | 2055 | CG | PRO | A | 286 | 24598 | 22633 | 27759 | −4497 | 9741 | −3340 | C |
| ATOM | 2056 | CD | PRO | A | 286 | 69.989 | −88.539 | 109.263 | 1.00 | 192.75 | | C |
| ANISOU | 2056 | CD | PRO | A | 286 | 24304 | 21594 | 27339 | −4517 | 10179 | −3429 | C |
| ATOM | 2057 | N | THR | A | 287 | 67.288 | −87.366 | 106.696 | 1.00 | 193.95 | | N |
| ANISOU | 2057 | N | THR | A | 287 | 23112 | 23315 | 27265 | −5147 | 9423 | −4582 | N |
| ATOM | 2058 | CA | THR | A | 287 | 66.350 | −86.544 | 105.919 | 1.00 | 193.71 | | C |
| ANISOU | 2058 | CA | THR | A | 287 | 22583 | 23910 | 27107 | −5312 | 9169 | −5000 | C |
| ATOM | 2059 | C | THR | A | 287 | 66.946 | −85.166 | 105.559 | 1.00 | 198.55 | | C |
| ANISOU | 2059 | C | THR | A | 287 | 23088 | 24849 | 27502 | −5055 | 8700 | −4919 | C |
| ATOM | 2060 | O | THR | A | 287 | 67.963 | −84.749 | 106.131 | 1.00 | 198.54 | | O |
| ANISOU | 2060 | O | THR | A | 287 | 23400 | 24487 | 27549 | −4933 | 8861 | −4751 | O |
| ATOM | 2061 | CB | THR | A | 287 | 64.968 | −86.445 | 106.608 | 1.00 | 196.94 | | C |
| ANISOU | 2061 | CB | THR | A | 287 | 22821 | 24632 | 27376 | −5186 | 8928 | −4835 | C |
| ATOM | 2062 | OG1 | THR | A | 287 | 65.123 | −86.060 | 107.974 | 1.00 | 194.03 | | O |
| ANISOU | 2062 | OG1 | THR | A | 287 | 22738 | 23884 | 27101 | −5193 | 9370 | −4777 | O |
| ATOM | 2063 | CG2 | THR | A | 287 | 64.169 | −87.728 | 106.515 | 1.00 | 194.52 | | C |
| ANISOU | 2063 | CG2 | THR | A | 287 | 22465 | 24251 | 27193 | −5381 | 9113 | −4893 | C |
| ATOM | 2064 | N | ARG | A | 288 | 66.314 | −84.483 | 104.578 | 1.00 | 195.09 | | N |
| ANISOU | 2064 | N | ARG | A | 288 | 22163 | 24963 | 27000 | −5330 | 8645 | −5487 | N |
| ATOM | 2065 | CA | ARG | A | 288 | 66.714 | −83.166 | 104.072 | 1.00 | 194.75 | | C |
| ANISOU | 2065 | CA | ARG | A | 288 | 21887 | 25282 | 26825 | −5338 | 8475 | −5762 | C |
| ATOM | 2066 | C | ARG | A | 288 | 66.582 | −82.061 | 105.127 | 1.00 | 197.45 | | C |
| ANISOU | 2066 | C | ARG | A | 288 | 22307 | 25666 | 27050 | −5119 | 8375 | −5575 | C |
| ATOM | 2067 | O | ARG | A | 288 | 67.380 | −81.119 | 105.117 | 1.00 | 196.85 | | O |
| ANISOU | 2067 | O | ARG | A | 288 | 22259 | 25589 | 26945 | −5104 | 8391 | −5687 | O |
| ATOM | 2068 | CB | ARG | A | 288 | 65.904 | −82.809 | 102.816 | 1.00 | 195.56 | | C |
| ANISOU | 2068 | CB | ARG | A | 288 | 21465 | 26119 | 26721 | −5361 | 8045 | −6028 | C |
| ATOM | 2069 | N | GLU | A | 289 | 65.571 | −82.181 | 106.025 | 1.00 | 192.97 | | N |
| ANISOU | 2069 | N | GLU | A | 289 | 21693 | 25049 | 26578 | −5340 | 8772 | −5787 | N |
| ATOM | 2070 | CA | GLU | A | 289 | 65.286 | −81.230 | 107.106 | 1.00 | 192.19 | | C |
| ANISOU | 2070 | CA | GLU | A | 289 | 21640 | 24944 | 26440 | −5305 | 8918 | −5816 | C |
| ATOM | 2071 | C | GLU | A | 289 | 66.255 | −81.375 | 108.280 | 1.00 | 194.47 | | C |
| ANISOU | 2071 | C | GLU | A | 289 | 22520 | 24541 | 26827 | −5047 | 9119 | −5298 | C |
| ATOM | 2072 | O | GLU | A | 289 | 66.779 | −80.364 | 108.751 | 1.00 | 194.31 | | O |
| ANISOU | 2072 | O | GLU | A | 289 | 22612 | 24440 | 26778 | −4975 | 9182 | −5309 | O |
| ATOM | 2073 | CB | GLU | A | 289 | 63.828 | −81.346 | 107.580 | 1.00 | 193.55 | | C |
| ANISOU | 2073 | CB | GLU | A | 289 | 21605 | 25418 | 26518 | −5258 | 8792 | −5760 | C |
| ATOM | 2074 | N | SER | A | 290 | 66.522 | −82.628 | 108.728 | 1.00 | 189.32 | | N |
| ANISOU | 2074 | N | SER | A | 290 | 22222 | 23252 | 26457 | −5234 | 9729 | −5258 | N |
| ATOM | 2075 | CA | SER | A | 290 | 67.455 | −82.935 | 109.822 | 1.00 | 188.35 | | C |
| ANISOU | 2075 | CA | SER | A | 290 | 22686 | 22399 | 26481 | −5046 | 10137 | −4839 | C |
| ATOM | 2076 | C | SER | A | 290 | 68.915 | −82.602 | 109.452 | 1.00 | 190.45 | | C |
| ANISOU | 2076 | C | SER | A | 290 | 23194 | 22424 | 26746 | −4839 | 10024 | −4622 | C |
| ATOM | 2077 | O | SER | A | 290 | 69.809 | −82.739 | 110.292 | 1.00 | 190.01 | | O |
| ANISOU | 2077 | O | SER | A | 290 | 23627 | 21796 | 26772 | −4598 | 10307 | −4213 | O |
| ATOM | 2078 | CB | SER | A | 290 | 67.317 | −84.391 | 110.261 | 1.00 | 192.02 | | C |
| ANISOU | 2078 | CB | SER | A | 290 | 23383 | 22638 | 26937 | −4813 | 10008 | −4313 | C |
| ATOM | 2079 | OG | SER | A | 290 | 67.865 | −85.292 | 109.313 | 1.00 | 201.27 | | O |
| ANISOU | 2079 | OG | SER | A | 290 | 24506 | 24055 | 27914 | −4461 | 9204 | −3841 | O |
| ATOM | 2080 | N | GLN | A | 291 | 69.139 | −82.161 | 108.192 | 1.00 | 185.47 | | N |
| ANISOU | 2080 | N | GLN | A | 291 | 22228 | 22038 | 26203 | −5236 | 10157 | −5266 | N |
| ATOM | 2081 | CA | GLN | A | 291 | 70.424 | −81.725 | 107.648 | 1.00 | 184.44 | | C |
| ANISOU | 2081 | CA | GLN | A | 291 | 22237 | 21695 | 26146 | −5223 | 10237 | −5310 | C |
| ATOM | 2082 | C | GLN | A | 291 | 70.540 | −80.193 | 107.775 | 1.00 | 186.16 | | C |
| ANISOU | 2082 | C | GLN | A | 291 | 22292 | 22233 | 26208 | −5155 | 10002 | −5447 | C |
| ATOM | 2083 | O | GLN | A | 291 | 71.606 | −79.689 | 108.145 | 1.00 | 186.08 | | O |
| ANISOU | 2083 | O | GLN | A | 291 | 22634 | 21796 | 26272 | −4983 | 10178 | −5220 | O |
| ATOM | 2084 | CB | GLN | A | 291 | 70.590 | −82.194 | 106.185 | 1.00 | 185.80 | | C |
| ANISOU | 2084 | CB | GLN | A | 291 | 22092 | 22241 | 26262 | −5335 | 9887 | −5516 | C |
| ATOM | 2085 | CG | GLN | A | 291 | 71.962 | −81.891 | 105.552 | 1.00 | 200.73 | | C |
| ANISOU | 2085 | CG | GLN | A | 291 | 24123 | 24350 | 27797 | −4571 | 8756 | −4646 | C |
| ATOM | 2086 | CD | GLN | A | 291 | 73.134 | −82.524 | 106.275 | 1.00 | 217.86 | | C |
| ANISOU | 2086 | CD | GLN | A | 291 | 26760 | 26415 | 29601 | −3579 | 7676 | −3289 | C |
| ATOM | 2087 | OE1 | GLN | A | 291 | 73.383 | −83.731 | 106.174 | 1.00 | 213.36 | | O |
| ANISOU | 2087 | OE1 | GLN | A | 291 | 26369 | 25462 | 29237 | −3747 | 8097 | −3334 | O |
| ATOM | 2088 | NE2 | GLN | A | 291 | 73.881 | −81.716 | 107.015 | 1.00 | 208.43 | | N |
| ANISOU | 2088 | NE2 | GLN | A | 291 | 25885 | 24607 | 28701 | −3813 | 8566 | −3583 | N |
| ATOM | 2089 | N | ALA | A | 292 | 69.433 | −79.464 | 107.486 | 1.00 | 180.29 | | N |
| ANISOU | 2089 | N | ALA | A | 292 | 20999 | 22078 | 25425 | −5615 | 10142 | −6223 | N |
| ATOM | 2090 | CA | ALA | A | 292 | 69.342 | −78.002 | 107.597 | 1.00 | 178.73 | | C |
| ANISOU | 2090 | CA | ALA | A | 292 | 20524 | 22285 | 25100 | −5719 | 10073 | −6607 | C |
| ATOM | 2091 | C | ALA | A | 292 | 69.332 | −77.586 | 109.071 | 1.00 | 178.47 | | C |
| ANISOU | 2091 | C | ALA | A | 292 | 20932 | 21730 | 25148 | −5552 | 10422 | −6290 | C |
| ATOM | 2092 | O | ALA | A | 292 | 69.821 | −76.505 | 109.404 | 1.00 | 177.91 | | O |
| ANISOU | 2092 | O | ALA | A | 292 | 20936 | 21606 | 25055 | −5514 | 10483 | −6354 | O |
| ATOM | 2093 | CB | ALA | A | 292 | 68.087 | −77.499 | 106.904 | 1.00 | 179.54 | | C |
| ANISOU | 2093 | CB | ALA | A | 292 | 19909 | 23412 | 24897 | −5823 | 9560 | −7034 | C |
| ATOM | 2094 | N | LEU | A | 293 | 68.766 | −78.452 | 109.944 | 1.00 | 171.85 | | N |
| ANISOU | 2094 | N | LEU | A | 293 | 20382 | 20340 | 24572 | −5774 | 11169 | −6336 | N |
| ATOM | 2095 | CA | LEU | A | 293 | 68.715 | −78.267 | 111.394 | 1.00 | 170.18 | | C |
| ANISOU | 2095 | CA | LEU | A | 293 | 20667 | 19534 | 24460 | −5602 | 11641 | −5991 | C |
| ATOM | 2096 | C | LEU | A | 293 | 70.123 | −78.477 | 111.972 | 1.00 | 170.03 | | C |
| ANISOU | 2096 | C | LEU | A | 293 | 21385 | 18616 | 24602 | −5216 | 12022 | −5349 | C |
| ATOM | 2097 | O | LEU | A | 293 | 70.552 | −77.704 | 112.827 | 1.00 | 169.85 | | O |
| ANISOU | 2097 | O | LEU | A | 293 | 21685 | 18404 | 24446 | −4790 | 11880 | −4864 | O |
| ATOM | 2098 | CB | LEU | A | 293 | 67.708 | −79.238 | 112.032 | 1.00 | 170.26 | | C |
| ANISOU | 2098 | CB | LEU | A | 293 | 20724 | 19481 | 24486 | −5584 | 11747 | −5807 | C |
| ATOM | 2099 | N | ARG | A | 294 | 70.856 | −79.491 | 111.463 | 1.00 | 163.21 | | N |
| ANISOU | 2099 | N | ARG | A | 294 | 20796 | 17156 | 24061 | −5407 | 12658 | −5423 | N |
| ATOM | 2100 | CA | ARG | A | 294 | 72.233 | −79.813 | 111.854 | 1.00 | 161.59 | | C |
| ANISOU | 2100 | CA | ARG | A | 294 | 21279 | 16113 | 24003 | −4980 | 13066 | −4779 | C |
| ATOM | 2101 | C | ARG | A | 294 | 73.173 | −78.688 | 111.424 | 1.00 | 160.13 | | C |
| ANISOU | 2101 | C | ARG | A | 294 | 20933 | 16490 | 23421 | −4448 | 11900 | −4335 | C |
| ATOM | 2102 | O | ARG | A | 294 | 74.168 | −78.429 | 112.099 | 1.00 | 159.63 | | O |
| ANISOU | 2102 | O | ARG | A | 294 | 21303 | 16162 | 23187 | −3794 | 11616 | −3545 | O |
| ATOM | 2103 | CB | ARG | A | 294 | 72.675 | −81.138 | 111.210 | 1.00 | 163.48 | | C |
| ANISOU | 2103 | CB | ARG | A | 294 | 21565 | 16256 | 24296 | −4912 | 12937 | −4571 | C |
| ATOM | 2104 | CG | ARG | A | 294 | 73.836 | −81.816 | 111.926 | 1.00 | 175.68 | | C |
| ANISOU | 2104 | CG | ARG | A | 294 | 23555 | 17700 | 25494 | −3943 | 12140 | −3326 | C |
| ATOM | 2105 | CD | ARG | A | 294 | 74.540 | −82.809 | 111.027 | 1.00 | 185.08 | | C |
| ANISOU | 2105 | CD | ARG | A | 294 | 24573 | 19270 | 26477 | −3682 | 11257 | −2980 | C |
| ATOM | 2106 | NE | ARG | A | 294 | 73.907 | −84.129 | 111.007 | 1.00 | 195.21 | | N |
| ANISOU | 2106 | NE | ARG | A | 294 | 25581 | 21132 | 27460 | −3484 | 10338 | −2632 | N |
| ATOM | 2107 | CZ | ARG | A | 294 | 74.129 | −85.090 | 111.900 | 1.00 | 212.12 | | C |
| ANISOU | 2107 | CZ | ARG | A | 294 | 27664 | 23898 | 29032 | −2753 | 8935 | −1682 | C |
| ATOM | 2108 | NH1 | ARG | A | 294 | 74.928 | −84.871 | 112.936 | 1.00 | 199.41 | | N |
| ANISOU | 2108 | NH1 | ARG | A | 294 | 26726 | 21289 | 27751 | −2690 | 10208 | −1569 | N |
| ATOM | 2109 | NH2 | ARG | A | 294 | 73.521 | −86.262 | 111.786 | 1.00 | 202.56 | | N |
| ANISOU | 2109 | NH2 | ARG | A | 294 | 26568 | 22231 | 28164 | −3152 | 9774 | −1987 | N |
| ATOM | 2110 | N | GLU | A | 295 | 72.841 | −78.023 | 110.301 | 1.00 | 152.61 | | N |
| ANISOU | 2110 | N | GLU | A | 295 | 19334 | 16347 | 22304 | −4723 | 11264 | −4864 | N |
| ATOM | 2111 | CA | GLU | A | 295 | 73.575 | −76.898 | 109.719 | 1.00 | 150.43 | | C |
| ANISOU | 2111 | CA | GLU | A | 295 | 18827 | 16655 | 21673 | −4307 | 10212 | −4564 | C |
| ATOM | 2112 | C | GLU | A | 295 | 73.647 | −75.687 | 110.673 | 1.00 | 147.97 | | C |
| ANISOU | 2112 | C | GLU | A | 295 | 18619 | 16593 | 21010 | −3728 | 9505 | −3978 | C |
| ATOM | 2113 | O | GLU | A | 295 | 74.673 | −75.010 | 110.737 | 1.00 | 147.51 | | O |
| ANISOU | 2113 | O | GLU | A | 295 | 18717 | 16591 | 20739 | −3217 | 8891 | −3430 | O |
| ATOM | 2114 | CB | GLU | A | 295 | 72.911 | −76.487 | 108.392 | 1.00 | 151.98 | | C |
| ANISOU | 2114 | CB | GLU | A | 295 | 18280 | 17717 | 21748 | −4735 | 9830 | −5310 | C |
| ATOM | 2115 | CG | GLU | A | 295 | 73.831 | −75.757 | 107.423 | 1.00 | 163.19 | | C |
| ANISOU | 2115 | CG | GLU | A | 295 | 19506 | 19567 | 22930 | −4429 | 9038 | −5106 | C |
| ATOM | 2116 | CD | GLU | A | 295 | 74.760 | −76.605 | 106.572 | 1.00 | 181.13 | | C |
| ANISOU | 2116 | CD | GLU | A | 295 | 21911 | 21489 | 25423 | −4574 | 9330 | −5179 | C |
| ATOM | 2117 | OE1 | GLU | A | 295 | 74.826 | −77.840 | 106.771 | 1.00 | 183.69 | | O |
| ANISOU | 2117 | OE1 | GLU | A | 295 | 22558 | 21118 | 26117 | −4875 | 10215 | −5322 | O |
| ATOM | 2118 | OE2 | GLU | A | 295 | 75.420 | −76.022 | 105.685 | 1.00 | 166.79 | | O |
| ANISOU | 2118 | OE2 | GLU | A | 295 | 19882 | 20077 | 23414 | −4379 | 8710 | −5090 | O |
| ATOM | 2119 | N | LYS | A | 296 | 72.563 | −75.423 | 111.404 | 1.00 | 139.17 | | N |
| ANISOU | 2119 | N | LYS | A | 296 | 17403 | 15631 | 19845 | −3840 | 9626 | −4138 | N |
| ATOM | 2120 | CA | LYS | A | 296 | 72.485 | −74.303 | 112.325 | 1.00 | 136.53 | | C |
| ANISOU | 2120 | CA | LYS | A | 296 | 17141 | 15536 | 19199 | −3363 | 9040 | −3678 | C |
| ATOM | 2121 | C | LYS | A | 296 | 73.020 | −74.668 | 113.697 | 1.00 | 136.24 | | C |
| ANISOU | 2121 | C | LYS | A | 296 | 17745 | 14841 | 19181 | −2944 | 9384 | −3022 | C |
| ATOM | 2122 | O | LYS | A | 296 | 73.885 | −73.951 | 114.197 | 1.00 | 133.89 | | O |
| ANISOU | 2122 | O | LYS | A | 296 | 17653 | 14590 | 18628 | −2396 | 8835 | −2445 | O |
| ATOM | 2123 | CB | LYS | A | 296 | 71.046 | −73.775 | 112.392 | 1.00 | 138.58 | | C |
| ANISOU | 2123 | CB | LYS | A | 296 | 16937 | 16360 | 19357 | −3652 | 8961 | −4172 | C |
| ATOM | 2124 | CG | LYS | A | 296 | 70.586 | −73.160 | 111.080 | 1.00 | 147.03 | | C |
| ANISOU | 2124 | CG | LYS | A | 296 | 17327 | 18278 | 20262 | −3873 | 8459 | −4704 | C |
| ATOM | 2125 | CD | LYS | A | 296 | 69.082 | −73.180 | 110.948 | 1.00 | 150.90 | | C |
| ANISOU | 2125 | CD | LYS | A | 296 | 17298 | 19299 | 20739 | −4329 | 8693 | −5397 | C |
| ATOM | 2126 | CE | LYS | A | 296 | 68.634 | −72.216 | 109.879 | 1.00 | 157.60 | | C |
| ANISOU | 2126 | CE | LYS | A | 296 | 17475 | 21146 | 21260 | −4290 | 8022 | −5725 | C |
| ATOM | 2127 | NZ | LYS | A | 296 | 67.165 | −72.017 | 109.899 | 1.00 | 166.06 | | N |
| ANISOU | 2127 | NZ | LYS | A | 296 | 18008 | 22871 | 22218 | −4589 | 8131 | −6307 | N |
| ATOM | 2128 | N | GLN | A | 297 | 72.526 | −75.799 | 114.295 | 1.00 | 132.28 | | N |
| ANISOU | 2128 | N | GLN | A | 297 | 17548 | 13745 | 18968 | −3193 | 10337 | −3126 | N |
| ATOM | 2129 | CA | GLN | A | 297 | 72.904 | −76.332 | 115.628 | 1.00 | 131.31 | | C |
| ANISOU | 2129 | CA | GLN | A | 297 | 18056 | 12973 | 18861 | −2777 | 10851 | −2504 | C |
| ATOM | 2130 | C | GLN | A | 297 | 74.414 | −76.306 | 115.849 | 1.00 | 131.76 | | C |
| ANISOU | 2130 | C | GLN | A | 297 | 18494 | 12816 | 18751 | −2148 | 10564 | −1790 | C |
| ATOM | 2131 | O | GLN | A | 297 | 74.857 | −76.164 | 116.994 | 1.00 | 132.76 | | O |
| ANISOU | 2131 | O | GLN | A | 297 | 18999 | 12783 | 18661 | −1601 | 10541 | −1182 | O |
| ATOM | 2132 | CB | GLN | A | 297 | 72.447 | −77.792 | 115.808 | 1.00 | 132.86 | | C |
| ANISOU | 2132 | CB | GLN | A | 297 | 18562 | 12433 | 19488 | −3166 | 12073 | −2738 | C |
| ATOM | 2133 | CG | GLN | A | 297 | 70.966 | −78.019 | 116.053 | 1.00 | 160.97 | | C |
| ANISOU | 2133 | CG | GLN | A | 297 | 21897 | 16036 | 23230 | −3724 | 12603 | −3340 | C |
| ATOM | 2134 | CD | GLN | A | 297 | 70.607 | −79.494 | 115.977 | 1.00 | 187.54 | | C |
| ANISOU | 2134 | CD | GLN | A | 297 | 24904 | 20127 | 26226 | −3368 | 11352 | −2802 | C |
| ATOM | 2135 | OE1 | GLN | A | 297 | 71.154 | −80.267 | 115.170 | 1.00 | 183.99 | | O |
| ANISOU | 2135 | OE1 | GLN | A | 297 | 24504 | 19427 | 25979 | −3536 | 11615 | −2922 | O |
| ATOM | 2136 | NE2 | GLN | A | 297 | 69.668 | −79.919 | 116.814 | 1.00 | 180.36 | | N |
| ANISOU | 2136 | NE2 | GLN | A | 297 | 24144 | 18905 | 25479 | −3578 | 12058 | −2940 | N |
| ATOM | 2137 | N | VAL | A | 298 | 75.193 | −76.494 | 114.760 | 1.00 | 123.03 | | N |
| ANISOU | 2137 | N | VAL | A | 298 | 17270 | 11743 | 17732 | −2228 | 10384 | −1890 | N |
| ATOM | 2138 | CA | VAL | A | 298 | 76.648 | −76.512 | 114.817 | 1.00 | 120.63 | | C |
| ANISOU | 2138 | CA | VAL | A | 298 | 17261 | 11289 | 17284 | −1678 | 10119 | −1288 | C |
| ATOM | 2139 | C | VAL | A | 298 | 77.191 | −75.106 | 115.013 | 1.00 | 121.57 | | C |
| ANISOU | 2139 | C | VAL | A | 298 | 17191 | 12007 | 16995 | −1260 | 9079 | −993 | C |
| ATOM | 2140 | O | VAL | A | 298 | 78.102 | −74.921 | 115.816 | 1.00 | 121.77 | | O |
| ANISOU | 2140 | O | VAL | A | 298 | 17510 | 11982 | 16776 | −688 | 8886 | −404 | O |
| ATOM | 2141 | CB | VAL | A | 298 | 77.376 | −77.279 | 113.660 | 1.00 | 123.25 | | C |
| ANISOU | 2141 | CB | VAL | A | 298 | 17589 | 11374 | 17864 | −1875 | 10364 | −1449 | C |
| ATOM | 2142 | CG1 | VAL | A | 298 | 77.117 | −78.778 | 113.727 | 1.00 | 122.84 | | C |
| ANISOU | 2142 | CG1 | VAL | A | 298 | 17896 | 10549 | 18227 | −2152 | 11551 | −1578 | C |
| ATOM | 2143 | CG2 | VAL | A | 298 | 77.063 | −76.722 | 112.274 | 1.00 | 122.70 | | C |
| ANISOU | 2143 | CG2 | VAL | A | 298 | 16933 | 11873 | 17816 | −2337 | 9833 | −2069 | C |
| ATOM | 2144 | N | CYS | A | 299 | 76.622 | −74.121 | 114.305 | 1.00 | 114.76 | | N |
| ANISOU | 2144 | N | CYS | A | 299 | 15833 | 11728 | 16043 | −1528 | 8462 | −1412 | N |
| ATOM | 2145 | CA | CYS | A | 299 | 77.078 | −72.733 | 114.330 | 1.00 | 112.67 | | C |
| ANISOU | 2145 | CA | CYS | A | 299 | 15372 | 11987 | 15450 | −1203 | 7551 | −1212 | C |
| ATOM | 2146 | C | CYS | A | 299 | 76.959 | −72.063 | 115.693 | 1.00 | 112.49 | | C |
| ANISOU | 2146 | C | CYS | A | 299 | 15530 | 12052 | 15157 | −819 | 7351 | −848 | C |
| ATOM | 2147 | O | CYS | A | 299 | 77.892 | −71.375 | 116.103 | 1.00 | 112.05 | | O |
| ANISOU | 2147 | O | CYS | A | 299 | 15562 | 12167 | 14846 | −391 | 6857 | −467 | O |
| ATOM | 2148 | CB | CYS | A | 299 | 76.392 | −71.918 | 113.244 | 1.00 | 112.45 | | C |
| ANISOU | 2148 | CB | CYS | A | 299 | 14804 | 12523 | 15398 | −1536 | 7083 | −1719 | C |
| ATOM | 2149 | SG | CYS | A | 299 | 77.036 | −72.222 | 111.587 | 1.00 | 115.80 | | S |
| ANISOU | 2149 | SG | CYS | A | 299 | 14971 | 13065 | 15961 | −1771 | 6951 | −2000 | S |
| ATOM | 2150 | N | TYR | A | 300 | 75.836 | −72.257 | 116.386 | 1.00 | 105.79 | | N |
| ANISOU | 2150 | N | TYR | A | 300 | 14717 | 11119 | 14361 | −991 | 7744 | −1001 | N |
| ATOM | 2151 | CA | TYR | A | 300 | 75.623 | −71.677 | 117.712 | 1.00 | 104.56 | | C |
| ANISOU | 2151 | CA | TYR | A | 300 | 14733 | 11048 | 13950 | −651 | 7612 | −688 | C |
| ATOM | 2152 | C | TYR | A | 300 | 76.472 | −72.406 | 118.772 | 1.00 | 108.69 | | C |
| ANISOU | 2152 | C | TYR | A | 300 | 15762 | 11176 | 14360 | −164 | 8000 | −101 | C |
| ATOM | 2153 | O | TYR | A | 300 | 76.955 | −71.769 | 119.706 | 1.00 | 107.44 | | O |
| ANISOU | 2153 | O | TYR | A | 300 | 15719 | 11232 | 13870 | 283 | 7646 | 268 | O |
| ATOM | 2154 | CB | TYR | A | 300 | 74.124 | −71.653 | 118.069 | 1.00 | 104.54 | | C |
| ANISOU | 2154 | CB | TYR | A | 300 | 14582 | 11102 | 14035 | −993 | 7919 | −1054 | C |
| ATOM | 2155 | CG | TYR | A | 300 | 73.278 | −70.805 | 117.136 | 1.00 | 104.69 | | C |
| ANISOU | 2155 | CG | TYR | A | 300 | 14061 | 11658 | 14058 | −1341 | 7481 | −1574 | C |
| ATOM | 2156 | CD1 | TYR | A | 300 | 72.797 | −71.320 | 115.935 | 1.00 | 106.93 | | C |
| ANISOU | 2156 | CD1 | TYR | A | 300 | 14018 | 12036 | 14573 | −1824 | 7695 | −2101 | C |
| ATOM | 2157 | CD2 | TYR | A | 300 | 72.949 | −69.491 | 117.457 | 1.00 | 104.44 | | C |
| ANISOU | 2157 | CD2 | TYR | A | 300 | 13833 | 12075 | 13774 | −1160 | 6890 | −1543 | C |
| ATOM | 2158 | CE1 | TYR | A | 300 | 72.015 | −70.548 | 115.076 | 1.00 | 107.28 | | C |
| ANISOU | 2158 | CE1 | TYR | A | 300 | 13528 | 12688 | 14544 | −2057 | 7296 | −2550 | C |
| ATOM | 2159 | CE2 | TYR | A | 300 | 72.153 | −68.714 | 116.612 | 1.00 | 104.66 | | C |
| ANISOU | 2159 | CE2 | TYR | A | 300 | 13378 | 12618 | 13770 | −1382 | 6541 | −1955 | C |
| ATOM | 2160 | CZ | TYR | A | 300 | 71.682 | −69.250 | 115.424 | 1.00 | 109.13 | | C |
| ANISOU | 2160 | CZ | TYR | A | 300 | 13599 | 13350 | 14515 | −1803 | 6731 | −2443 | C |
| ATOM | 2161 | OH | TYR | A | 300 | 70.903 | −68.497 | 114.579 | 1.00 | 105.73 | | O |
| ANISOU | 2161 | OH | TYR | A | 300 | 12655 | 13537 | 13981 | −1942 | 6388 | −2823 | O |
| ATOM | 2162 | N | HIS | A | 301 | 76.695 | −73.731 | 118.584 | 1.00 | 106.42 | | N |
| ANISOU | 2162 | N | HIS | A | 301 | 15758 | 10347 | 14328 | −228 | 8744 | −22 | N |
| ATOM | 2163 | CA | HIS | A | 301 | 77.531 | −74.604 | 119.431 | 1.00 | 105.95 | | C |
| ANISOU | 2163 | CA | HIS | A | 301 | 16200 | 9878 | 14176 | 294 | 9246 | 579 | C |
| ATOM | 2164 | C | HIS | A | 301 | 78.993 | −74.113 | 119.411 | 1.00 | 107.32 | | C |
| ANISOU | 2164 | C | HIS | A | 301 | 16378 | 10360 | 14038 | 808 | 8623 | 987 | C |
| ATOM | 2165 | O | HIS | A | 301 | 79.696 | −74.198 | 120.422 | 1.00 | 107.40 | | O |
| ANISOU | 2165 | O | HIS | A | 301 | 16657 | 10422 | 13730 | 1402 | 8649 | 1519 | O |
| ATOM | 2166 | CB | HIS | A | 301 | 77.466 | −76.058 | 118.931 | 1.00 | 106.83 | | C |
| ANISOU | 2166 | CB | HIS | A | 301 | 16571 | 9320 | 14698 | 47 | 10192 | 494 | C |
| ATOM | 2167 | CG | HIS | A | 301 | 77.869 | −77.070 | 119.957 | 1.00 | 110.05 | | C |
| ANISOU | 2167 | CG | HIS | A | 301 | 17551 | 9204 | 15061 | 559 | 10981 | 1094 | C |
| ATOM | 2168 | ND1 | HIS | A | 301 | 79.195 | −77.251 | 120.309 | 1.00 | 111.66 | | N |
| ANISOU | 2168 | ND1 | HIS | A | 301 | 18006 | 9435 | 14987 | 1239 | 10867 | 1716 | N |
| ATOM | 2169 | CD2 | HIS | A | 301 | 77.105 | −77.932 | 120.673 | 1.00 | 111.12 | | C |
| ANISOU | 2169 | CD2 | HIS | A | 301 | 18035 | 8806 | 15378 | 512 | 11916 | 1166 | C |
| ATOM | 2170 | CE1 | HIS | A | 301 | 79.197 | −78.202 | 121.230 | 1.00 | 110.76 | | C |
| ANISOU | 2170 | CE1 | HIS | A | 301 | 18388 | 8835 | 14860 | 1644 | 11720 | 2190 | C |
| ATOM | 2171 | NE2 | HIS | A | 301 | 77.963 | −78.640 | 121.486 | 1.00 | 110.78 | | N |
| ANISOU | 2171 | NE2 | HIS | A | 301 | 18494 | 8445 | 15152 | 1222 | 12396 | 1892 | N |
| ATOM | 2172 | N | VAL | A | 302 | 79.440 | −73.601 | 118.257 | 1.00 | 100.86 | | N |
| ANISOU | 2172 | N | VAL | A | 302 | 15236 | 9796 | 13291 | 586 | 8081 | 718 | N |
| ATOM | 2173 | CA | VAL | A | 302 | 80.767 | −73.021 | 118.124 | 1.00 | 99.98 | | C |
| ANISOU | 2173 | CA | VAL | A | 302 | 15057 | 10015 | 12916 | 975 | 7462 | 996 | C |
| ATOM | 2174 | C | VAL | A | 302 | 80.768 | −71.696 | 118.887 | 1.00 | 103.71 | | C |
| ANISOU | 2174 | C | VAL | A | 302 | 15359 | 11020 | 13027 | 1186 | 6790 | 1047 | C |
| ATOM | 2175 | O | VAL | A | 302 | 81.661 | −71.492 | 119.701 | 1.00 | 104.51 | | O |
| ANISOU | 2175 | O | VAL | A | 302 | 15577 | 11346 | 12786 | 1690 | 6586 | 1435 | O |
| ATOM | 2176 | CB | VAL | A | 302 | 81.207 | −72.848 | 116.651 | 1.00 | 103.54 | | C |
| ANISOU | 2176 | CB | VAL | A | 302 | 15224 | 10553 | 13563 | 662 | 7129 | 687 | C |
| ATOM | 2177 | CG1 | VAL | A | 302 | 82.485 | −72.024 | 116.549 | 1.00 | 103.23 | | C |
| ANISOU | 2177 | CG1 | VAL | A | 302 | 15061 | 10912 | 13248 | 1010 | 6431 | 907 | C |
| ATOM | 2178 | CG2 | VAL | A | 302 | 81.397 | −74.199 | 115.974 | 1.00 | 103.28 | | C |
| ANISOU | 2178 | CG2 | VAL | A | 302 | 15390 | 9992 | 13862 | 501 | 7820 | 662 | C |
| ATOM | 2179 | N | LEU | A | 303 | 79.740 | −70.829 | 118.668 | 1.00 | 98.20 | | N |
| ANISOU | 2179 | N | LEU | A | 303 | 14377 | 10545 | 12388 | 817 | 6500 | 643 | N |
| ATOM | 2180 | CA | LEU | A | 303 | 79.567 | −69.531 | 119.345 | 1.00 | 96.66 | | C |
| ANISOU | 2180 | CA | LEU | A | 303 | 14026 | 10790 | 11910 | 947 | 5948 | 622 | C |
| ATOM | 2181 | C | LEU | A | 303 | 79.742 | −69.668 | 120.854 | 1.00 | 99.24 | | C |
| ANISOU | 2181 | C | LEU | A | 303 | 14629 | 11161 | 11917 | 1403 | 6119 | 1015 | C |
| ATOM | 2182 | O | LEU | A | 303 | 80.471 | −68.877 | 121.449 | 1.00 | 98.22 | | O |
| ANISOU | 2182 | O | LEU | A | 303 | 14441 | 11424 | 11455 | 1720 | 5658 | 1166 | O |
| ATOM | 2183 | CB | LEU | A | 303 | 78.197 | −68.908 | 118.989 | 1.00 | 95.98 | | C |
| ANISOU | 2183 | CB | LEU | A | 303 | 13671 | 10826 | 11971 | 519 | 5865 | 179 | C |
| ATOM | 2184 | CG | LEU | A | 303 | 77.721 | −67.672 | 119.743 | 1.00 | 98.59 | | C |
| ANISOU | 2184 | CG | LEU | A | 303 | 13883 | 11507 | 12072 | 609 | 5466 | 129 | C |
| ATOM | 2185 | CD1 | LEU | A | 303 | 78.469 | −66.431 | 119.298 | 1.00 | 98.15 | | C |
| ANISOU | 2185 | CD1 | LEU | A | 303 | 13609 | 11799 | 11885 | 694 | 4804 | 76 | C |
| ATOM | 2186 | CD2 | LEU | A | 303 | 76.266 | −67.465 | 119.501 | 1.00 | 98.46 | | C |
| ANISOU | 2186 | CD2 | LEU | A | 303 | 13665 | 11532 | 12212 | 241 | 5604 | −242 | C |
| ATOM | 2187 | N | GLY | A | 304 | 79.120 | −70.697 | 121.430 | 1.00 | 95.77 | | N |
| ANISOU | 2187 | N | GLY | A | 304 | 14479 | 10336 | 11574 | 1432 | 6818 | 1161 | N |
| ATOM | 2188 | CA | GLY | A | 304 | 79.222 | −71.021 | 122.847 | 1.00 | 96.01 | | C |
| ANISOU | 2188 | CA | GLY | A | 304 | 14815 | 10370 | 11294 | 1917 | 7113 | 1590 | C |
| ATOM | 2189 | C | GLY | A | 304 | 80.650 | −71.317 | 123.253 | 1.00 | 100.94 | | C |
| ANISOU | 2189 | C | GLY | A | 304 | 15590 | 11171 | 11590 | 2515 | 7023 | 2067 | C |
| ATOM | 2190 | O | GLY | A | 304 | 81.197 | −70.650 | 124.136 | 1.00 | 101.59 | | O |
| ANISOU | 2190 | O | GLY | A | 304 | 15616 | 11744 | 11238 | 2909 | 6628 | 2250 | O |
| ATOM | 2191 | N | LEU | A | 305 | 81.278 | −72.286 | 122.559 | 1.00 | 96.89 | | N |
| ANISOU | 2191 | N | LEU | A | 305 | 15226 | 10313 | 11273 | 2570 | 7375 | 2224 | N |
| ATOM | 2192 | CA | LEU | A | 305 | 82.657 | −72.720 | 122.777 | 1.00 | 96.30 | | C |
| ANISOU | 2192 | CA | LEU | A | 305 | 15282 | 10388 | 10918 | 3155 | 7359 | 2687 | C |
| ATOM | 2193 | C | LEU | A | 305 | 83.639 | −71.535 | 122.645 | 1.00 | 100.95 | | C |
| ANISOU | 2193 | C | LEU | A | 305 | 15511 | 11645 | 11202 | 3269 | 6472 | 2577 | C |
| ATOM | 2194 | O | LEU | A | 305 | 84.553 | −71.415 | 123.467 | 1.00 | 101.11 | | O |
| ANISOU | 2194 | O | LEU | A | 305 | 15537 | 12121 | 10760 | 3830 | 6284 | 2908 | O |
| ATOM | 2195 | CB | LEU | A | 305 | 83.004 | −73.859 | 121.800 | 1.00 | 95.98 | | C |
| ANISOU | 2195 | CB | LEU | A | 305 | 15419 | 9803 | 11247 | 3049 | 7880 | 2752 | C |
| ATOM | 2196 | N | VAL | A | 306 | 83.403 | −70.638 | 121.643 | 1.00 | 96.70 | | N |
| ANISOU | 2196 | N | VAL | A | 306 | 14643 | 11196 | 10902 | 2742 | 5968 | 2093 | N |
| ATOM | 2197 | CA | VAL | A | 306 | 84.184 | −69.430 | 121.333 | 1.00 | 95.59 | | C |
| ANISOU | 2197 | CA | VAL | A | 306 | 14164 | 11567 | 10590 | 2711 | 5211 | 1889 | C |
| ATOM | 2198 | C | VAL | A | 306 | 84.100 | −68.463 | 122.512 | 1.00 | 98.40 | | C |
| ANISOU | 2198 | C | VAL | A | 306 | 14418 | 12420 | 10551 | 2909 | 4873 | 1869 | C |
| ATOM | 2199 | O | VAL | A | 306 | 85.122 | −67.933 | 122.941 | 1.00 | 97.26 | | O |
| ANISOU | 2199 | O | VAL | A | 306 | 14120 | 12782 | 10050 | 3208 | 4482 | 1932 | O |
| ATOM | 2200 | CB | VAL | A | 306 | 83.745 | −68.781 | 119.977 | 1.00 | 98.98 | | C |
| ANISOU | 2200 | CB | VAL | A | 306 | 14327 | 11885 | 11394 | 2131 | 4908 | 1421 | C |
| ATOM | 2201 | CG1 | VAL | A | 306 | 84.098 | −67.293 | 119.890 | 1.00 | 98.80 | | C |
| ANISOU | 2201 | CG1 | VAL | A | 306 | 13997 | 12315 | 11226 | 2039 | 4235 | 1163 | C |
| ATOM | 2202 | CG2 | VAL | A | 306 | 84.326 | −69.534 | 118.788 | 1.00 | 98.45 | | C |
| ANISOU | 2202 | CG2 | VAL | A | 306 | 14273 | 11535 | 11597 | 2017 | 5056 | 1429 | C |
| ATOM | 2203 | N | HIS | A | 307 | 82.884 | −68.266 | 123.046 | 1.00 | 95.05 | | N |
| ANISOU | 2203 | N | HIS | A | 307 | 14059 | 11874 | 10181 | 2731 | 5052 | 1750 | N |
| ATOM | 2204 | CA | HIS | A | 307 | 82.621 | −67.386 | 124.180 | 1.00 | 94.96 | | C |
| ANISOU | 2204 | CA | HIS | A | 307 | 13974 | 12278 | 9829 | 2872 | 4804 | 1697 | C |
| ATOM | 2205 | C | HIS | A | 307 | 83.188 | −67.944 | 125.490 | 1.00 | 101.62 | | C |
| ANISOU | 2205 | C | HIS | A | 307 | 15006 | 13418 | 10187 | 3504 | 5017 | 2143 | C |
| ATOM | 2206 | O | HIS | A | 307 | 83.536 | −67.175 | 126.386 | 1.00 | 100.78 | | O |
| ANISOU | 2206 | O | HIS | A | 307 | 14751 | 13872 | 9669 | 3727 | 4678 | 2100 | O |
| ATOM | 2207 | CB | HIS | A | 307 | 81.123 | −67.081 | 124.280 | 1.00 | 95.11 | | C |
| ANISOU | 2207 | CB | HIS | A | 307 | 14006 | 12061 | 10071 | 2500 | 4969 | 1446 | C |
| ATOM | 2208 | CG | HIS | A | 307 | 80.622 | −66.091 | 123.267 | 1.00 | 97.80 | | C |
| ANISOU | 2208 | CG | HIS | A | 307 | 14067 | 12393 | 10700 | 2013 | 4600 | 1002 | C |
| ATOM | 2209 | ND1 | HIS | A | 307 | 79.373 | −65.519 | 123.389 | 1.00 | 99.27 | | N |
| ANISOU | 2209 | ND1 | HIS | A | 307 | 14179 | 12536 | 11004 | 1734 | 4619 | 748 | N |
| ATOM | 2210 | CD2 | HIS | A | 307 | 81.219 | −65.592 | 122.158 | 1.00 | 98.75 | | C |
| ANISOU | 2210 | CD2 | HIS | A | 307 | 13978 | 12567 | 10978 | 1825 | 4242 | 813 | C |
| ATOM | 2211 | CE1 | HIS | A | 307 | 79.250 | −64.699 | 122.360 | 1.00 | 98.30 | | C |
| ANISOU | 2211 | CE1 | HIS | A | 307 | 13807 | 12460 | 11084 | 1433 | 4285 | 443 | C |
| ATOM | 2212 | NE2 | HIS | A | 307 | 80.333 | −64.711 | 121.593 | 1.00 | 98.39 | | N |
| ANISOU | 2212 | NE2 | HIS | A | 307 | 13740 | 12513 | 11130 | 1470 | 4060 | 473 | N |
| ATOM | 2213 | N | ALA | A | 308 | 83.318 | −69.274 | 125.585 | 1.00 | 101.15 | | N |
| ANISOU | 2213 | N | ALA | A | 308 | 15262 | 13011 | 10158 | 3816 | 5609 | 2563 | N |
| ATOM | 2214 | CA | ALA | A | 308 | 83.933 | −69.925 | 126.740 | 1.00 | 102.18 | | C |
| ANISOU | 2214 | CA | ALA | A | 308 | 15595 | 13433 | 9795 | 4538 | 5884 | 3086 | C |
| ATOM | 2215 | C | ALA | A | 308 | 85.486 | −69.881 | 126.608 | 1.00 | 107.22 | | C |
| ANISOU | 2215 | C | ALA | A | 308 | 16046 | 14597 | 10096 | 4951 | 5520 | 3248 | C |
| ATOM | 2216 | O | ALA | A | 308 | 86.177 | −69.974 | 127.629 | 1.00 | 107.81 | | O |
| ANISOU | 2216 | O | ALA | A | 308 | 16103 | 15256 | 9602 | 5577 | 5482 | 3566 | O |
| ATOM | 2217 | CB | ALA | A | 308 | 83.442 | −71.359 | 126.859 | 1.00 | 103.17 | | C |
| ANISOU | 2217 | CB | ALA | A | 308 | 16172 | 12908 | 10119 | 4733 | 6765 | 3496 | C |
| ATOM | 2218 | N | LEU | A | 309 | 86.023 | −69.707 | 125.357 | 1.00 | 102.55 | | N |
| ANISOU | 2218 | N | LEU | A | 309 | 15277 | 13865 | 9823 | 4612 | 5241 | 3009 | N |
| ATOM | 2219 | CA | LEU | A | 309 | 87.462 | −69.593 | 125.059 | 1.00 | 101.56 | | C |
| ANISOU | 2219 | CA | LEU | A | 309 | 14929 | 14208 | 9452 | 4896 | 4872 | 3077 | C |
| ATOM | 2220 | C | LEU | A | 309 | 87.956 | −68.141 | 125.184 | 1.00 | 105.74 | | C |
| ANISOU | 2220 | C | LEU | A | 309 | 15022 | 15400 | 9756 | 4695 | 4137 | 2622 | C |
| ATOM | 2221 | O | LEU | A | 309 | 88.942 | −67.903 | 125.883 | 1.00 | 105.54 | | O |
| ANISOU | 2221 | O | LEU | A | 309 | 14788 | 16101 | 9213 | 5128 | 3872 | 2701 | O |
| ATOM | 2222 | CB | LEU | A | 309 | 87.806 | −70.182 | 123.672 | 1.00 | 101.14 | | C |
| ANISOU | 2222 | CB | LEU | A | 309 | 14926 | 13647 | 9856 | 4641 | 5003 | 3056 | C |
| ATOM | 2223 | CG | LEU | A | 309 | 89.253 | −69.990 | 123.141 | 1.00 | 104.73 | | C |
| ANISOU | 2223 | CG | LEU | A | 309 | 15120 | 14524 | 10149 | 4822 | 4594 | 3051 | C |
| ATOM | 2224 | CD1 | LEU | A | 309 | 90.223 | −70.939 | 123.816 | 1.00 | 104.84 | | C |
| ANISOU | 2224 | CD1 | LEU | A | 309 | 15267 | 14850 | 9717 | 5623 | 4883 | 3623 | C |
| ATOM | 2225 | CD2 | LEU | A | 309 | 89.321 | −70.183 | 121.630 | 1.00 | 104.44 | | C |
| ANISOU | 2225 | CD2 | LEU | A | 309 | 15065 | 13975 | 10642 | 4356 | 4595 | 2846 | C |
| ATOM | 2226 | N | TRP | A | 310 | 87.296 | −67.186 | 124.491 | 1.00 | 102.17 | | N |
| ANISOU | 2226 | N | TRP | A | 310 | 14419 | 14722 | 9681 | 4057 | 3852 | 2137 | N |
| ATOM | 2227 | CA | TRP | A | 310 | 87.636 | −65.759 | 124.550 | 1.00 | 101.88 | | C |
| ANISOU | 2227 | CA | TRP | A | 310 | 14023 | 15161 | 9525 | 3799 | 3280 | 1673 | C |
| ATOM | 2228 | C | TRP | A | 310 | 86.420 | −64.968 | 125.079 | 1.00 | 101.93 | | C |
| ANISOU | 2228 | C | TRP | A | 310 | 14044 | 15086 | 9598 | 3505 | 3263 | 1413 | C |
| ATOM | 2229 | O | TRP | A | 310 | 85.280 | −65.280 | 124.717 | 1.00 | 100.98 | | O |
| ANISOU | 2229 | O | TRP | A | 310 | 14116 | 14420 | 9832 | 3243 | 3556 | 1420 | O |
| ATOM | 2230 | CB | TRP | A | 310 | 88.044 | −65.173 | 123.185 | 1.00 | 101.48 | | C |
| ANISOU | 2230 | CB | TRP | A | 310 | 13779 | 14922 | 9858 | 3355 | 2976 | 1347 | C |
| ATOM | 2231 | CG | TRP | A | 310 | 88.909 | −65.971 | 122.243 | 1.00 | 103.27 | | C |
| ANISOU | 2231 | CG | TRP | A | 310 | 14032 | 14975 | 10233 | 3457 | 3048 | 1543 | C |
| ATOM | 2232 | CD1 | TRP | A | 310 | 88.573 | −66.362 | 120.977 | 1.00 | 106.25 | | C |
| ANISOU | 2232 | CD1 | TRP | A | 310 | 14501 | 14782 | 11086 | 3126 | 3193 | 1503 | C |
| ATOM | 2233 | CD2 | TRP | A | 310 | 90.306 | −66.260 | 122.388 | 1.00 | 103.53 | | C |
| ANISOU | 2233 | CD2 | TRP | A | 310 | 13917 | 15488 | 9933 | 3863 | 2901 | 1708 | C |
| ATOM | 2234 | NE1 | TRP | A | 310 | 89.657 | −66.932 | 120.349 | 1.00 | 105.63 | | N |
| ANISOU | 2234 | NE1 | TRP | A | 310 | 14389 | 14730 | 11016 | 3302 | 3180 | 1663 | N |
| ATOM | 2235 | CE2 | TRP | A | 310 | 90.737 | −66.872 | 121.186 | 1.00 | 107.39 | | C |
| ANISOU | 2235 | CE2 | TRP | A | 310 | 14463 | 15587 | 10751 | 3761 | 2998 | 1801 | C |
| ATOM | 2236 | CE3 | TRP | A | 310 | 91.234 | −66.076 | 123.427 | 1.00 | 105.25 | | C |
| ANISOU | 2236 | CE3 | TRP | A | 310 | 13924 | 16498 | 9569 | 4319 | 2700 | 1768 | C |
| ATOM | 2237 | CZ2 | TRP | A | 310 | 92.042 | −67.326 | 121.003 | 1.00 | 107.19 | | C |
| ANISOU | 2237 | CZ2 | TRP | A | 310 | 14327 | 15878 | 10521 | 4114 | 2920 | 1990 | C |
| ATOM | 2238 | CZ3 | TRP | A | 310 | 92.529 | −66.527 | 123.245 | 1.00 | 107.27 | | C |
| ANISOU | 2238 | CZ3 | TRP | A | 310 | 14031 | 17137 | 9588 | 4685 | 2609 | 1943 | C |
| ATOM | 2239 | CH2 | TRP | A | 310 | 92.928 | −67.125 | 122.037 | 1.00 | 108.04 | | C |
| ANISOU | 2239 | CH2 | TRP | A | 310 | 14217 | 16781 | 10053 | 4578 | 2716 | 2059 | C |
| ATOM | 2240 | N | GLN | A | 311 | 86.664 | −63.929 | 125.898 | 1.00 | 95.68 | | N |
| ANISOU | 2240 | N | GLN | A | 311 | 13023 | 14858 | 8474 | 3521 | 2934 | 1137 | N |
| ATOM | 2241 | CA | GLN | A | 311 | 85.600 | −63.122 | 126.498 | 1.00 | 93.81 | | C |
| ANISOU | 2241 | CA | GLN | A | 311 | 12793 | 14592 | 8257 | 3286 | 2921 | 889 | C |
| ATOM | 2242 | C | GLN | A | 311 | 84.819 | −62.241 | 125.478 | 1.00 | 93.55 | | C |
| ANISOU | 2242 | C | GLN | A | 311 | 12703 | 14106 | 8735 | 2710 | 2806 | 523 | C |
| ATOM | 2243 | O | GLN | A | 311 | 85.412 | −61.348 | 124.874 | 1.00 | 94.50 | | O |
| ANISOU | 2243 | O | GLN | A | 311 | 12606 | 14332 | 8966 | 2471 | 2501 | 208 | O |
| ATOM | 2244 | CB | GLN | A | 311 | 86.152 | −62.283 | 127.659 | 1.00 | 95.32 | | C |
| ANISOU | 2244 | CB | GLN | A | 311 | 12740 | 15540 | 7937 | 3459 | 2642 | 656 | C |
| ATOM | 2245 | CG | GLN | A | 311 | 85.073 | −61.843 | 128.653 | 1.00 | 115.00 | | C |
| ANISOU | 2245 | CG | GLN | A | 311 | 15319 | 18067 | 10308 | 3419 | 2756 | 563 | C |
| ATOM | 2246 | CD | GLN | A | 311 | 85.292 | −60.446 | 129.193 | 1.00 | 132.75 | | C |
| ANISOU | 2246 | CD | GLN | A | 311 | 17278 | 20789 | 12373 | 3195 | 2432 | 38 | C |
| ATOM | 2247 | OE1 | GLN | A | 311 | 85.241 | −59.441 | 128.463 | 1.00 | 130.43 | | O |
| ANISOU | 2247 | OE1 | GLN | A | 311 | 16857 | 20278 | 12421 | 2744 | 2259 | −359 | O |
| ATOM | 2248 | NE2 | GLN | A | 311 | 85.522 | −60.353 | 130.491 | 1.00 | 119.24 | | N |
| ANISOU | 2248 | NE2 | GLN | A | 311 | 15464 | 19731 | 10110 | 3516 | 2395 | 19 | N |
| ATOM | 2249 | N | PRO | A | 312 | 83.485 | −62.437 | 125.315 | 1.00 | 85.16 | | N |
| ANISOU | 2249 | N | PRO | A | 312 | 11817 | 12585 | 7956 | 2508 | 3072 | 551 | N |
| ATOM | 2250 | CA | PRO | A | 312 | 82.727 | −61.622 | 124.345 | 1.00 | 82.74 | | C |
| ANISOU | 2250 | CA | PRO | A | 312 | 11423 | 11955 | 8061 | 2060 | 2973 | 244 | C |
| ATOM | 2251 | C | PRO | A | 312 | 82.476 | −60.176 | 124.751 | 1.00 | 80.71 | | C |
| ANISOU | 2251 | C | PRO | A | 312 | 11013 | 11901 | 7751 | 1880 | 2743 | −116 | C |
| ATOM | 2252 | O | PRO | A | 312 | 82.199 | −59.874 | 125.904 | 1.00 | 79.41 | | O |
| ANISOU | 2252 | O | PRO | A | 312 | 10868 | 11995 | 7308 | 2002 | 2768 | −159 | O |
| ATOM | 2253 | CB | PRO | A | 312 | 81.424 | −62.396 | 124.153 | 1.00 | 84.73 | | C |
| ANISOU | 2253 | CB | PRO | A | 312 | 11859 | 11778 | 8558 | 1947 | 3357 | 372 | C |
| ATOM | 2254 | CG | PRO | A | 312 | 81.264 | −63.168 | 125.383 | 1.00 | 90.37 | | C |
| ANISOU | 2254 | CG | PRO | A | 312 | 12775 | 12592 | 8970 | 2285 | 3648 | 660 | C |
| ATOM | 2255 | CD | PRO | A | 312 | 82.625 | −63.454 | 125.947 | 1.00 | 86.50 | | C |
| ANISOU | 2255 | CD | PRO | A | 312 | 12259 | 12524 | 8082 | 2697 | 3519 | 866 | C |
| ATOM | 2256 | N | ILE | A | 313 | 82.591 | −59.287 | 123.773 | 1.00 | 74.63 | | N |
| ANISOU | 2256 | N | ILE | A | 313 | 10106 | 10995 | 7255 | 1602 | 2563 | −369 | N |
| ATOM | 2257 | CA | ILE | A | 313 | 82.389 | −57.849 | 123.938 | 1.00 | 73.33 | | C |
| ANISOU | 2257 | CA | ILE | A | 313 | 9827 | 10902 | 7131 | 1407 | 2433 | −717 | C |
| ATOM | 2258 | C | ILE | A | 313 | 80.870 | −57.561 | 123.904 | 1.00 | 75.00 | | C |
| ANISOU | 2258 | C | ILE | A | 313 | 10123 | 10842 | 7533 | 1282 | 2615 | −740 | C |
| ATOM | 2259 | O | ILE | A | 313 | 80.156 | −58.102 | 123.044 | 1.00 | 74.17 | | O |
| ANISOU | 2259 | O | ILE | A | 313 | 10054 | 10453 | 7675 | 1201 | 2742 | −621 | O |
| ATOM | 2260 | CB | ILE | A | 313 | 83.221 | −57.010 | 122.896 | 1.00 | 76.08 | | C |
| ANISOU | 2260 | CB | ILE | A | 313 | 10026 | 11184 | 7696 | 1212 | 2250 | −939 | C |
| ATOM | 2261 | CG1 | ILE | A | 313 | 84.715 | −57.414 | 122.938 | 1.00 | 77.05 | | C |
| ANISOU | 2261 | CG1 | ILE | A | 313 | 10033 | 11626 | 7616 | 1340 | 2078 | −923 | C |
| ATOM | 2262 | CG2 | ILE | A | 313 | 83.063 | −55.498 | 123.089 | 1.00 | 75.33 | | C |
| ANISOU | 2262 | CG2 | ILE | A | 313 | 9858 | 11088 | 7676 | 1016 | 2232 | −1301 | C |
| ATOM | 2263 | CD1 | ILE | A | 313 | 85.618 | −56.806 | 121.908 | 1.00 | 83.12 | | C |
| ANISOU | 2263 | CD1 | ILE | A | 313 | 10666 | 12322 | 8596 | 1158 | 1938 | −1110 | C |
| ATOM | 2264 | N | PRO | A | 314 | 80.368 | −56.735 | 124.857 | 1.00 | 69.58 | | N |
| ANISOU | 2264 | N | PRO | A | 314 | 9438 | 10289 | 6709 | 1265 | 2641 | −921 | N |
| ATOM | 2265 | CA | PRO | A | 314 | 78.942 | −56.396 | 124.864 | 1.00 | 68.53 | | C |
| ANISOU | 2265 | CA | PRO | A | 314 | 9361 | 9944 | 6734 | 1175 | 2811 | −948 | C |
| ATOM | 2266 | C | PRO | A | 314 | 78.393 | −55.781 | 123.570 | 1.00 | 70.48 | | C |
| ANISOU | 2266 | C | PRO | A | 314 | 9533 | 9917 | 7328 | 1015 | 2819 | −1022 | C |
| ATOM | 2267 | O | PRO | A | 314 | 78.935 | −54.804 | 123.029 | 1.00 | 69.10 | | O |
| ANISOU | 2267 | O | PRO | A | 314 | 9287 | 9694 | 7273 | 933 | 2724 | −1188 | O |
| ATOM | 2268 | CB | PRO | A | 314 | 78.828 | −55.412 | 126.026 | 1.00 | 70.42 | | C |
| ANISOU | 2268 | CB | PRO | A | 314 | 9592 | 10396 | 6767 | 1175 | 2805 | −1184 | C |
| ATOM | 2269 | CG | PRO | A | 314 | 79.997 | −55.695 | 126.880 | 1.00 | 75.02 | | C |
| ANISOU | 2269 | CG | PRO | A | 314 | 10127 | 11389 | 6988 | 1325 | 2672 | −1210 | C |
| ATOM | 2270 | CD | PRO | A | 314 | 81.078 | −56.046 | 125.954 | 1.00 | 70.81 | | C |
| ANISOU | 2270 | CD | PRO | A | 314 | 9512 | 10843 | 6548 | 1316 | 2524 | −1153 | C |
| ATOM | 2271 | N | GLY | A | 315 | 77.295 | −56.383 | 123.117 | 1.00 | 66.68 | | N |
| ANISOU | 2271 | N | GLY | A | 315 | 9059 | 9293 | 6984 | 989 | 2974 | −906 | N |
| ATOM | 2272 | CA | GLY | A | 315 | 76.504 | −56.009 | 121.949 | 1.00 | 65.85 | | C |
| ANISOU | 2272 | CA | GLY | A | 315 | 8837 | 9059 | 7123 | 908 | 3011 | −944 | C |
| ATOM | 2273 | C | GLY | A | 315 | 77.174 | −56.261 | 120.628 | 1.00 | 68.35 | | C |
| ANISOU | 2273 | C | GLY | A | 315 | 9064 | 9303 | 7602 | 864 | 2901 | −898 | C |
| ATOM | 2274 | O | GLY | A | 315 | 76.983 | −55.477 | 119.694 | 1.00 | 68.80 | | O |
| ANISOU | 2274 | O | GLY | A | 315 | 9020 | 9316 | 7803 | 863 | 2869 | −951 | O |
| ATOM | 2275 | N | ARG | A | 316 | 77.936 | −57.367 | 120.526 | 1.00 | 62.61 | | N |
| ANISOU | 2275 | N | ARG | A | 316 | 8383 | 8565 | 6843 | 865 | 2877 | −773 | N |
| ATOM | 2276 | CA | ARG | A | 316 | 78.693 | −57.648 | 119.314 | 1.00 | 61.08 | | C |
| ANISOU | 2276 | CA | ARG | A | 316 | 8111 | 8305 | 6791 | 821 | 2769 | −735 | C |
| ATOM | 2277 | C | ARG | A | 316 | 78.337 | −58.986 | 118.651 | 1.00 | 65.83 | | C |
| ANISOU | 2277 | C | ARG | A | 316 | 8689 | 8824 | 7498 | 743 | 2920 | −645 | C |
| ATOM | 2278 | O | ARG | A | 316 | 79.024 | −59.399 | 117.710 | 1.00 | 66.85 | | O |
| ANISOU | 2278 | O | ARG | A | 316 | 8768 | 8900 | 7730 | 705 | 2851 | −609 | O |
| ATOM | 2279 | CB | ARG | A | 316 | 80.207 | −57.519 | 119.582 | 1.00 | 56.23 | | C |
| ANISOU | 2279 | CB | ARG | A | 316 | 7536 | 7758 | 6072 | 881 | 2591 | −725 | C |
| ATOM | 2280 | CG | ARG | A | 316 | 80.694 | −56.091 | 119.904 | 1.00 | 48.54 | | C |
| ANISOU | 2280 | CG | ARG | A | 316 | 6530 | 6845 | 5069 | 860 | 2483 | −928 | C |
| ATOM | 2281 | CD | ARG | A | 316 | 80.543 | −55.122 | 118.727 | 1.00 | 55.49 | | C |
| ANISOU | 2281 | CD | ARG | A | 316 | 7334 | 7564 | 6184 | 801 | 2484 | −1003 | C |
| ATOM | 2282 | NE | ARG | A | 316 | 81.413 | −55.500 | 117.602 | 1.00 | 69.72 | | N |
| ANISOU | 2282 | NE | ARG | A | 316 | 9074 | 9309 | 8107 | 774 | 2379 | −935 | N |
| ATOM | 2283 | CZ | ARG | A | 316 | 81.314 | −55.058 | 116.345 | 1.00 | 78.81 | | C |
| ANISOU | 2283 | CZ | ARG | A | 316 | 10155 | 10348 | 9442 | 773 | 2391 | −913 | C |
| ATOM | 2284 | NH1 | ARG | A | 316 | 80.335 | −54.232 | 115.998 | 1.00 | 55.16 | | N |
| ANISOU | 2284 | NH1 | ARG | A | 316 | 7135 | 7304 | 6521 | 841 | 2514 | −923 | N |
| ATOM | 2285 | NH2 | ARG | A | 316 | 82.158 | −55.485 | 115.417 | 1.00 | 71.56 | | N |
| ANISOU | 2285 | NH2 | ARG | A | 316 | 9187 | 9396 | 8608 | 747 | 2294 | −853 | N |
| ATOM | 2286 | N | VAL | A | 317 | 77.237 | −59.630 | 119.078 | 1.00 | 61.07 | | N |
| ANISOU | 2286 | N | VAL | A | 317 | 8111 | 8201 | 6894 | 685 | 3163 | −652 | N |
| ATOM | 2287 | CA | VAL | A | 317 | 76.808 | −60.853 | 118.405 | 1.00 | 59.64 | | C |
| ANISOU | 2287 | CA | VAL | A | 317 | 7884 | 7923 | 6854 | 535 | 3392 | −663 | C |
| ATOM | 2288 | C | VAL | A | 317 | 75.883 | −60.456 | 117.249 | 1.00 | 64.76 | | C |
| ANISOU | 2288 | C | VAL | A | 317 | 8254 | 8725 | 7626 | 405 | 3366 | −864 | C |
| ATOM | 2289 | O | VAL | A | 317 | 74.908 | −59.711 | 117.444 | 1.00 | 64.94 | | O |
| ANISOU | 2289 | O | VAL | A | 317 | 8169 | 8900 | 7607 | 430 | 3369 | −963 | O |
| ATOM | 2290 | CB | VAL | A | 317 | 76.229 | −61.948 | 119.324 | 1.00 | 61.68 | | C |
| ANISOU | 2290 | CB | VAL | A | 317 | 8308 | 8046 | 7080 | 506 | 3762 | −592 | C |
| ATOM | 2291 | CG1 | VAL | A | 317 | 75.923 | −63.213 | 118.527 | 1.00 | 60.93 | | C |
| ANISOU | 2291 | CG1 | VAL | A | 317 | 8173 | 7792 | 7186 | 290 | 4076 | −667 | C |
| ATOM | 2292 | CG2 | VAL | A | 317 | 77.177 | −62.251 | 120.482 | 1.00 | 61.19 | | C |
| ANISOU | 2292 | CG2 | VAL | A | 317 | 8502 | 7939 | 6810 | 750 | 3776 | −340 | C |
| ATOM | 2293 | N | LYS | A | 318 | 76.268 | −60.859 | 116.033 | 1.00 | 61.03 | | N |
| ANISOU | 2293 | N | LYS | A | 318 | 7651 | 8265 | 7271 | 315 | 3319 | −911 | N |
| ATOM | 2294 | CA | LYS | A | 318 | 75.518 | −60.548 | 114.830 | 1.00 | 60.41 | | C |
| ANISOU | 2294 | CA | LYS | A | 318 | 7264 | 8444 | 7245 | 245 | 3276 | −1094 | C |
| ATOM | 2295 | C | LYS | A | 318 | 75.094 | −61.854 | 114.177 | 1.00 | 65.79 | | C |
| ANISOU | 2295 | C | LYS | A | 318 | 7810 | 9145 | 8044 | −19 | 3532 | −1281 | C |
| ATOM | 2296 | O | LYS | A | 318 | 75.929 | −62.624 | 113.714 | 1.00 | 62.84 | | O |
| ANISOU | 2296 | O | LYS | A | 318 | 7512 | 8600 | 7765 | −96 | 3574 | −1237 | O |
| ATOM | 2297 | CB | LYS | A | 318 | 76.311 | −59.609 | 113.903 | 1.00 | 60.99 | | C |
| ANISOU | 2297 | CB | LYS | A | 318 | 7267 | 8584 | 7322 | 397 | 2997 | −1020 | C |
| ATOM | 2298 | CG | LYS | A | 318 | 76.540 | −58.222 | 114.500 | 1.00 | 55.56 | | C |
| ANISOU | 2298 | CG | LYS | A | 318 | 6692 | 7858 | 6561 | 602 | 2855 | −922 | C |
| ATOM | 2299 | CD | LYS | A | 318 | 77.669 | −57.479 | 113.839 | 1.00 | 53.94 | | C |
| ANISOU | 2299 | CD | LYS | A | 318 | 6523 | 7569 | 6403 | 708 | 2672 | −834 | C |
| ATOM | 2300 | CE | LYS | A | 318 | 78.814 | −57.160 | 114.782 | 1.00 | 62.69 | | C |
| ANISOU | 2300 | CE | LYS | A | 318 | 7854 | 8485 | 7482 | 730 | 2585 | −772 | C |
| ATOM | 2301 | NZ | LYS | A | 318 | 79.889 | −58.211 | 114.801 | 1.00 | 71.73 | | N |
| ANISOU | 2301 | NZ | LYS | A | 318 | 9082 | 9537 | 8637 | 661 | 2538 | −700 | N |
| ATOM | 2302 | N | ASP | A | 319 | 73.793 | −62.150 | 114.263 | 1.00 | 66.66 | | N |
| ANISOU | 2302 | N | ASP | A | 319 | 7731 | 9444 | 8154 | −179 | 3755 | −1515 | N |
| ATOM | 2303 | CA | ASP | A | 319 | 73.208 | −63.368 | 113.726 | 1.00 | 68.23 | | C |
| ANISOU | 2303 | CA | ASP | A | 319 | 7760 | 9687 | 8479 | −511 | 4088 | −1809 | C |
| ATOM | 2304 | C | ASP | A | 319 | 72.598 | −63.114 | 112.352 | 1.00 | 75.71 | | C |
| ANISOU | 2304 | C | ASP | A | 319 | 8244 | 11147 | 9375 | −588 | 3971 | −2104 | C |
| ATOM | 2305 | O | ASP | A | 319 | 71.508 | −62.536 | 112.239 | 1.00 | 75.96 | | O |
| ANISOU | 2305 | O | ASP | A | 319 | 7980 | 11605 | 9278 | −544 | 3936 | −2276 | O |
| ATOM | 2306 | CB | ASP | A | 319 | 72.183 | −63.956 | 114.712 | 1.00 | 70.40 | | C |
| ANISOU | 2306 | CB | ASP | A | 319 | 8091 | 9869 | 8789 | −686 | 4476 | −1943 | C |
| ATOM | 2307 | CG | ASP | A | 319 | 71.546 | −65.285 | 114.316 | 1.00 | 81.96 | | C |
| ANISOU | 2307 | CG | ASP | A | 319 | 9413 | 11296 | 10433 | −1102 | 4955 | −2309 | C |
| ATOM | 2308 | OD1 | ASP | A | 319 | 71.897 | −65.823 | 113.232 | 1.00 | 79.08 | | O |
| ANISOU | 2308 | OD1 | ASP | A | 319 | 8887 | 10998 | 10163 | −1277 | 4986 | −2490 | O |
| ATOM | 2309 | OD2 | ASP | A | 319 | 70.694 | −65.792 | 115.094 | 1.00 | 90.32 | | O |
| ANISOU | 2309 | OD2 | ASP | A | 319 | 10522 | 12246 | 11549 | −1277 | 5341 | −2444 | O |
| ATOM | 2310 | N | TYR | A | 320 | 73.331 | −63.538 | 111.308 | 1.00 | 74.37 | | N |
| ANISOU | 2310 | N | TYR | A | 320 | 8001 | 10984 | 9274 | −663 | 3908 | −2149 | N |
| ATOM | 2311 | CA | TYR | A | 320 | 72.922 | −63.436 | 109.907 | 1.00 | 75.01 | | C |
| ANISOU | 2311 | CA | TYR | A | 320 | 7632 | 11598 | 9270 | −720 | 3802 | −2430 | C |
| ATOM | 2312 | C | TYR | A | 320 | 72.340 | −64.753 | 109.390 | 1.00 | 85.10 | | C |
| ANISOU | 2312 | C | TYR | A | 320 | 8663 | 13000 | 10669 | −1176 | 4184 | −2901 | C |
| ATOM | 2313 | O | TYR | A | 320 | 71.877 | −64.818 | 108.254 | 1.00 | 85.97 | | O |
| ANISOU | 2313 | O | TYR | A | 320 | 8329 | 13660 | 10678 | −1282 | 4140 | −3238 | O |
| ATOM | 2314 | CB | TYR | A | 320 | 74.066 | −62.923 | 109.020 | 1.00 | 74.46 | | C |
| ANISOU | 2314 | CB | TYR | A | 320 | 7606 | 11513 | 9173 | −498 | 3486 | −2203 | C |
| ATOM | 2315 | CG | TYR | A | 320 | 74.534 | −61.544 | 109.418 | 1.00 | 73.19 | | C |
| ANISOU | 2315 | CG | TYR | A | 320 | 7634 | 11262 | 8911 | −101 | 3188 | −1835 | C |
| ATOM | 2316 | CD2 | TYR | A | 320 | 75.828 | −61.332 | 109.873 | 1.00 | 73.43 | | C |
| ANISOU | 2316 | CD2 | TYR | A | 320 | 8030 | 10843 | 9027 | 15 | 3062 | −1531 | C |
| ATOM | 2317 | CD1 | TYR | A | 320 | 73.679 | −60.452 | 109.349 | 1.00 | 74.19 | | C |
| ANISOU | 2317 | CD1 | TYR | A | 320 | 7561 | 11769 | 8858 | 155 | 3079 | −1818 | C |
| ATOM | 2318 | CE2 | TYR | A | 320 | 76.256 | −60.065 | 110.265 | 1.00 | 73.92 | | C |
| ANISOU | 2318 | CE2 | TYR | A | 320 | 8250 | 10812 | 9026 | 305 | 2860 | −1284 | C |
| ATOM | 2319 | CE1 | TYR | A | 320 | 74.092 | −59.184 | 109.744 | 1.00 | 73.68 | | C |
| ANISOU | 2319 | CE1 | TYR | A | 320 | 7705 | 11545 | 8747 | 488 | 2909 | −1511 | C |
| ATOM | 2320 | CZ | TYR | A | 320 | 75.381 | −58.994 | 110.202 | 1.00 | 79.25 | | C |
| ANISOU | 2320 | CZ | TYR | A | 320 | 8767 | 11776 | 9566 | 526 | 2812 | −1277 | C |
| ATOM | 2321 | OH | TYR | A | 320 | 75.782 | −57.738 | 110.583 | 1.00 | 81.26 | | O |
| ANISOU | 2321 | OH | TYR | A | 320 | 9204 | 11874 | 9799 | 784 | 2707 | −1064 | O |
| ATOM | 2322 | N | ILE | A | 321 | 72.342 | −65.797 | 110.230 | 1.00 | 84.12 | | N |
| ANISOU | 2322 | N | ILE | A | 321 | 8820 | 12388 | 10752 | −1439 | 4606 | −2943 | N |
| ATOM | 2323 | CA | ILE | A | 321 | 71.723 | −67.077 | 109.901 | 1.00 | 84.38 | | C |
| ANISOU | 2323 | CA | ILE | A | 321 | 8673 | 12427 | 10963 | −1931 | 5113 | −3433 | C |
| ATOM | 2324 | C | ILE | A | 321 | 70.186 | −66.808 | 110.019 | 1.00 | 94.13 | | C |
| ANISOU | 2324 | C | ILE | A | 321 | 9483 | 14217 | 12067 | −2081 | 5212 | −3835 | C |
| ATOM | 2325 | O | ILE | A | 321 | 69.439 | −66.987 | 109.053 | 1.00 | 94.25 | | O |
| ANISOU | 2325 | O | ILE | A | 321 | 8967 | 14851 | 11991 | −2317 | 5256 | −4341 | O |
| ATOM | 2326 | CB | ILE | A | 321 | 72.277 | −68.175 | 110.860 | 1.00 | 85.82 | | C |
| ANISOU | 2326 | CB | ILE | A | 321 | 9373 | 11829 | 11407 | −2062 | 5600 | −3242 | C |
| ATOM | 2327 | CG1 | ILE | A | 321 | 73.764 | −68.456 | 110.569 | 1.00 | 84.52 | | C |
| ANISOU | 2327 | CG1 | ILE | A | 321 | 9526 | 11261 | 11326 | −1890 | 5487 | −2890 | C |
| ATOM | 2328 | CG2 | ILE | A | 321 | 71.440 | −69.450 | 110.821 | 1.00 | 85.69 | | C |
| ANISOU | 2328 | CG2 | ILE | A | 321 | 9243 | 11692 | 11622 | −2596 | 6285 | −3755 | C |
| ATOM | 2329 | CD1 | ILE | A | 321 | 74.560 | −69.027 | 111.706 | 1.00 | 81.53 | | C |
| ANISOU | 2329 | CD1 | ILE | A | 321 | 9703 | 10222 | 11054 | −1715 | 5753 | −2453 | C |
| ATOM | 2330 | N | ALA | A | 322 | 69.761 | −66.261 | 111.167 | 1.00 | 94.70 | | N |
| ANISOU | 2330 | N | ALA | A | 322 | 9749 | 14153 | 12079 | −1891 | 5192 | −3603 | N |
| ATOM | 2331 | CA | ALA | A | 322 | 68.371 | −65.900 | 111.450 | 1.00 | 96.69 | | C |
| ANISOU | 2331 | CA | ALA | A | 322 | 9652 | 14888 | 12198 | −1962 | 5263 | −3901 | C |
| ATOM | 2332 | C | ALA | A | 322 | 67.892 | −64.645 | 110.695 | 1.00 | 105.62 | | C |
| ANISOU | 2332 | C | ALA | A | 322 | 10343 | 16783 | 13005 | −1614 | 4788 | −3909 | C |
| ATOM | 2333 | O | ALA | A | 322 | 66.688 | −64.504 | 110.468 | 1.00 | 106.43 | | O |
| ANISOU | 2333 | O | ALA | A | 322 | 9970 | 17517 | 12951 | −1706 | 4851 | −4291 | O |
| ATOM | 2334 | CB | ALA | A | 322 | 68.181 | −65.707 | 112.943 | 1.00 | 97.27 | | C |
| ANISOU | 2334 | CB | ALA | A | 322 | 10122 | 14526 | 12310 | −1834 | 5398 | −3602 | C |
| ATOM | 2335 | N | VAL | A | 323 | 68.813 | −63.702 | 110.385 | 1.00 | 104.03 | | N |
| ANISOU | 2335 | N | VAL | A | 323 | 10312 | 16518 | 12695 | −1184 | 4358 | −3468 | N |
| ATOM | 2336 | CA | VAL | A | 323 | 68.523 | −62.451 | 109.660 | 1.00 | 104.64 | | C |
| ANISOU | 2336 | CA | VAL | A | 323 | 10075 | 17206 | 12479 | −756 | 3975 | −3356 | C |
| ATOM | 2337 | C | VAL | A | 323 | 69.537 | −62.301 | 108.513 | 1.00 | 111.71 | | C |
| ANISOU | 2337 | C | VAL | A | 323 | 10947 | 18158 | 13340 | −606 | 3727 | −3218 | C |
| ATOM | 2338 | O | VAL | A | 323 | 70.494 | −61.521 | 108.630 | 1.00 | 110.84 | | O |
| ANISOU | 2338 | O | VAL | A | 323 | 11170 | 17701 | 13241 | −278 | 3484 | −2771 | O |
| ATOM | 2339 | CB | VAL | A | 323 | 68.490 | −61.185 | 110.565 | 1.00 | 108.18 | | C |
| ANISOU | 2339 | CB | VAL | A | 323 | 10791 | 17479 | 12833 | −325 | 3781 | −2927 | C |
| ATOM | 2340 | CG1 | VAL | A | 323 | 68.127 | −59.937 | 109.754 | 1.00 | 107.68 | | C |
| ANISOU | 2340 | CG1 | VAL | A | 323 | 10419 | 18014 | 12480 | 155 | 3505 | −2794 | C |
| ATOM | 2341 | CG2 | VAL | A | 323 | 67.535 | −61.363 | 111.739 | 1.00 | 108.20 | | C |
| ANISOU | 2341 | CG2 | VAL | A | 323 | 10842 | 17398 | 12870 | −473 | 4031 | −3049 | C |
| ATOM | 2342 | N | PRO | A | 324 | 69.363 | −63.037 | 107.393 | 1.00 | 110.91 | | N |
| ANISOU | 2342 | N | PRO | A | 324 | 10442 | 18500 | 13197 | −864 | 3806 | −3630 | N |
| ATOM | 2343 | CA | PRO | A | 324 | 70.334 | −62.917 | 106.298 | 1.00 | 111.52 | | C |
| ANISOU | 2343 | CA | PRO | A | 324 | 10502 | 18634 | 13235 | −717 | 3582 | −3495 | C |
| ATOM | 2344 | C | PRO | A | 324 | 70.303 | −61.550 | 105.645 | 1.00 | 117.53 | | C |
| ANISOU | 2344 | C | PRO | A | 324 | 11096 | 19865 | 13694 | −143 | 3235 | −3192 | C |
| ATOM | 2345 | O | PRO | A | 324 | 69.255 | −60.887 | 105.632 | 1.00 | 116.15 | | O |
| ANISOU | 2345 | O | PRO | A | 324 | 10596 | 20274 | 13263 | 105 | 3193 | −3254 | O |
| ATOM | 2346 | CB | PRO | A | 324 | 69.958 | −64.055 | 105.339 | 1.00 | 113.28 | | C |
| ANISOU | 2346 | CB | PRO | A | 324 | 10283 | 19295 | 13462 | −1170 | 3804 | −4096 | C |
| ATOM | 2347 | CG | PRO | A | 324 | 69.048 | −64.947 | 106.131 | 1.00 | 117.70 | | C |
| ANISOU | 2347 | CG | PRO | A | 324 | 10782 | 19757 | 14184 | −1638 | 4234 | −4512 | C |
| ATOM | 2348 | CD | PRO | A | 324 | 68.322 | −64.028 | 107.067 | 1.00 | 113.04 | | C |
| ANISOU | 2348 | CD | PRO | A | 324 | 10245 | 19241 | 13463 | −1337 | 4134 | −4282 | C |
| ATOM | 2349 | N | LYS | A | 325 | 71.485 | −61.115 | 105.161 | 1.00 | 116.44 | | N |
| ANISOU | 2349 | N | LYS | A | 325 | 11215 | 19428 | 13599 | 95 | 3031 | −2832 | N |
| ATOM | 2350 | CA | LYS | A | 325 | 71.683 | −59.826 | 104.503 | 1.00 | 116.74 | | C |
| ANISOU | 2350 | CA | LYS | A | 325 | 11202 | 19747 | 13408 | 658 | 2789 | −2475 | C |
| ATOM | 2351 | C | LYS | A | 325 | 70.932 | −59.831 | 103.161 | 1.00 | 124.99 | | C |
| ANISOU | 2351 | C | LYS | A | 325 | 11615 | 21784 | 14093 | 812 | 2728 | −2759 | C |
| ATOM | 2352 | O | LYS | A | 325 | 70.705 | −60.922 | 102.630 | 1.00 | 125.12 | | O |
| ANISOU | 2352 | O | LYS | A | 325 | 11311 | 22113 | 14114 | 394 | 2837 | −3239 | O |
| ATOM | 2353 | CB | LYS | A | 325 | 73.186 | −59.549 | 104.297 | 1.00 | 117.24 | | C |
| ANISOU | 2353 | CB | LYS | A | 325 | 11683 | 19214 | 13648 | 769 | 2656 | −2105 | C |
| ATOM | 2354 | CG | LYS | A | 325 | 73.997 | −59.457 | 105.581 | 1.00 | 116.08 | | C |
| ANISOU | 2354 | CG | LYS | A | 325 | 12090 | 18235 | 13779 | 675 | 2682 | −1842 | C |
| ATOM | 2355 | CD | LYS | A | 325 | 74.997 | −58.304 | 105.564 | 1.00 | 122.05 | | C |
| ANISOU | 2355 | CD | LYS | A | 325 | 13167 | 18630 | 14575 | 1029 | 2538 | −1417 | C |
| ATOM | 2356 | CE | LYS | A | 325 | 76.243 | −58.625 | 106.357 | 1.00 | 126.85 | | C |
| ANISOU | 2356 | CE | LYS | A | 325 | 14215 | 18542 | 15442 | 833 | 2521 | −1275 | C |
| ATOM | 2357 | NZ | LYS | A | 325 | 77.065 | −57.422 | 106.637 | 1.00 | 132.06 | | N |
| ANISOU | 2357 | NZ | LYS | A | 325 | 15170 | 18852 | 16155 | 1104 | 2442 | −958 | N |
| ATOM | 2358 | N | PRO | A | 326 | 70.498 | −58.666 | 102.587 | 1.00 | 124.02 | | N |
| ANISOU | 2358 | N | PRO | A | 326 | 11275 | 22216 | 13632 | 1415 | 2601 | −2501 | N |
| ATOM | 2359 | CA | PRO | A | 326 | 69.803 | −58.713 | 101.284 | 1.00 | 124.50 | | C |
| ANISOU | 2359 | CA | PRO | A | 326 | 10682 | 23355 | 13268 | 1625 | 2533 | −2768 | C |
| ATOM | 2360 | C | PRO | A | 326 | 70.644 | −59.360 | 100.167 | 1.00 | 128.37 | | C |
| ANISOU | 2360 | C | PRO | A | 326 | 11070 | 23940 | 13767 | 1453 | 2461 | −2909 | C |
| ATOM | 2361 | O | PRO | A | 326 | 70.081 | −59.837 | 99.188 | 1.00 | 127.90 | | O |
| ANISOU | 2361 | O | PRO | A | 326 | 10423 | 24769 | 13403 | 1393 | 2445 | −3329 | O |
| ATOM | 2362 | CB | PRO | A | 326 | 69.443 | −57.244 | 101.019 | 1.00 | 126.29 | | C |
| ANISOU | 2362 | CB | PRO | A | 326 | 10869 | 23946 | 13171 | 2417 | 2461 | −2285 | C |
| ATOM | 2363 | CG | PRO | A | 326 | 69.449 | −56.600 | 102.378 | 1.00 | 130.31 | | C |
| ANISOU | 2363 | CG | PRO | A | 326 | 11874 | 23705 | 13932 | 2469 | 2550 | −1978 | C |
| ATOM | 2364 | CD | PRO | A | 326 | 70.603 | −57.271 | 103.065 | 1.00 | 125.80 | | C |
| ANISOU | 2364 | CD | PRO | A | 326 | 11823 | 22152 | 13822 | 1957 | 2564 | −1969 | C |
| ATOM | 2365 | N | ASN | A | 327 | 71.981 | −59.462 | 100.363 | 1.00 | 125.09 | | N |
| ANISOU | 2365 | N | ASN | A | 327 | 11194 | 22638 | 13698 | 1324 | 2431 | −2621 | N |
| ATOM | 2366 | CA | ASN | A | 327 | 72.901 | −60.164 | 99.448 | 1.00 | 125.53 | | C |
| ANISOU | 2366 | CA | ASN | A | 327 | 11234 | 22625 | 13835 | 1104 | 2388 | −2742 | C |
| ATOM | 2367 | C | ASN | A | 327 | 72.941 | −61.702 | 99.714 | 1.00 | 129.77 | | C |
| ANISOU | 2367 | C | ASN | A | 327 | 11740 | 22914 | 14653 | 369 | 2590 | −3270 | C |
| ATOM | 2368 | O | ASN | A | 327 | 73.787 | −62.410 | 99.153 | 1.00 | 129.88 | | O |
| ANISOU | 2368 | O | ASN | A | 327 | 11832 | 22696 | 14820 | 118 | 2612 | −3371 | O |
| ATOM | 2369 | CB | ASN | A | 327 | 74.313 | −59.532 | 99.448 | 1.00 | 126.86 | | C |
| ANISOU | 2369 | CB | ASN | A | 327 | 11945 | 22037 | 14219 | 1337 | 2286 | −2205 | C |
| ATOM | 2370 | CG | ASN | A | 327 | 74.964 | −59.414 | 100.807 | 1.00 | 143.10 | | C |
| ANISOU | 2370 | CG | ASN | A | 327 | 14586 | 23135 | 16650 | 1176 | 2336 | −1951 | C |
| ATOM | 2371 | OD1 | ASN | A | 327 | 75.716 | −60.289 | 101.246 | 1.00 | 131.95 | | O |
| ANISOU | 2371 | OD1 | ASN | A | 327 | 13442 | 21156 | 15538 | 769 | 2395 | −2038 | O |
| ATOM | 2372 | ND2 | ASN | A | 327 | 74.697 | −58.310 | 101.497 | 1.00 | 135.11 | | N |
| ANISOU | 2372 | ND2 | ASN | A | 327 | 13772 | 21958 | 15604 | 1528 | 2334 | −1628 | N |
| ATOM | 2373 | N | GLY | A | 328 | 71.993 | −62.183 | 100.536 | 1.00 | 125.55 | | N |
| ANISOU | 2373 | N | GLY | A | 328 | 11089 | 22433 | 14180 | 54 | 2784 | −3597 | N |
| ATOM | 2374 | CA | GLY | A | 328 | 71.798 | −63.583 | 100.915 | 1.00 | 124.84 | | C |
| ANISOU | 2374 | CA | GLY | A | 328 | 10978 | 22092 | 14364 | −623 | 3105 | −4108 | C |
| ATOM | 2375 | C | GLY | A | 328 | 72.766 | −64.146 | 101.939 | 1.00 | 127.00 | | C |
| ANISOU | 2375 | C | GLY | A | 328 | 11903 | 21269 | 15083 | −879 | 3266 | −3858 | C |
| ATOM | 2376 | O | GLY | A | 328 | 72.732 | −65.350 | 102.218 | 1.00 | 127.59 | | O |
| ANISOU | 2376 | O | GLY | A | 328 | 12037 | 21030 | 15413 | −1393 | 3612 | −4210 | O |
| ATOM | 2377 | N | TYR | A | 329 | 73.628 | −63.279 | 102.510 | 1.00 | 120.26 | | N |
| ANISOU | 2377 | N | TYR | A | 329 | 11530 | 19853 | 14312 | −509 | 3060 | −3264 | N |
| ATOM | 2378 | CA | TYR | A | 329 | 74.659 | −63.636 | 103.484 | 1.00 | 117.93 | | C |
| ANISOU | 2378 | CA | TYR | A | 329 | 11821 | 18641 | 14348 | −628 | 3143 | −2964 | C |
| ATOM | 2379 | C | TYR | A | 329 | 74.133 | −64.188 | 104.831 | 1.00 | 118.08 | | C |
| ANISOU | 2379 | C | TYR | A | 329 | 12060 | 18261 | 14544 | −884 | 3430 | −3044 | C |
| ATOM | 2380 | O | TYR | A | 329 | 73.408 | −63.511 | 105.567 | 1.00 | 116.32 | | O |
| ANISOU | 2380 | O | TYR | A | 329 | 11834 | 18143 | 14218 | −715 | 3392 | −2953 | O |
| ATOM | 2381 | CB | TYR | A | 329 | 75.600 | −62.448 | 103.717 | 1.00 | 118.14 | | C |
| ANISOU | 2381 | CB | TYR | A | 329 | 12195 | 18331 | 14361 | −180 | 2854 | −2409 | C |
| ATOM | 2382 | CG | TYR | A | 329 | 76.747 | −62.742 | 104.653 | 1.00 | 118.64 | | C |
| ANISOU | 2382 | CG | TYR | A | 329 | 12781 | 17606 | 14689 | −253 | 2888 | −2124 | C |
| ATOM | 2383 | CD1 | TYR | A | 329 | 77.841 | −63.492 | 104.232 | 1.00 | 120.13 | | C |
| ANISOU | 2383 | CD1 | TYR | A | 329 | 13127 | 17472 | 15044 | −403 | 2921 | −2094 | C |
| ATOM | 2384 | CD2 | TYR | A | 329 | 76.760 | −62.239 | 105.949 | 1.00 | 119.18 | | C |
| ANISOU | 2384 | CD2 | TYR | A | 329 | 13161 | 17317 | 14804 | −136 | 2883 | −1883 | C |
| ATOM | 2385 | CE1 | TYR | A | 329 | 78.907 | −63.757 | 105.085 | 1.00 | 119.66 | | C |
| ANISOU | 2385 | CE1 | TYR | A | 329 | 13500 | 16799 | 15166 | −402 | 2945 | −1819 | C |
| ATOM | 2386 | CE2 | TYR | A | 329 | 77.822 | −62.497 | 106.813 | 1.00 | 119.93 | | C |
| ANISOU | 2386 | CE2 | TYR | A | 329 | 13674 | 16829 | 15065 | −154 | 2896 | −1638 | C |
| ATOM | 2387 | CZ | TYR | A | 329 | 78.902 | −63.246 | 106.370 | 1.00 | 124.43 | | C |
| ANISOU | 2387 | CZ | TYR | A | 329 | 14370 | 17135 | 15771 | −265 | 2920 | −1595 | C |
| ATOM | 2388 | OH | TYR | A | 329 | 79.961 | −63.497 | 107.207 | 1.00 | 121.54 | | O |
| ANISOU | 2388 | OH | TYR | A | 329 | 14368 | 16300 | 15511 | −219 | 2925 | −1343 | O |
| ATOM | 2389 | N | GLN | A | 330 | 74.570 | −65.421 | 105.145 | 1.00 | 112.04 | | N |
| ANISOU | 2389 | N | GLN | A | 330 | 11522 | 16998 | 14050 | −1258 | 3753 | −3174 | N |
| ATOM | 2390 | CA | GLN | A | 330 | 74.297 | −66.155 | 106.377 | 1.00 | 109.67 | | C |
| ANISOU | 2390 | CA | GLN | A | 330 | 11517 | 16202 | 13952 | −1491 | 4123 | −3194 | C |
| ATOM | 2391 | C | GLN | A | 330 | 75.632 | −66.474 | 107.048 | 1.00 | 108.28 | | C |
| ANISOU | 2391 | C | GLN | A | 330 | 11886 | 15291 | 13965 | −1379 | 4150 | −2761 | C |
| ATOM | 2392 | O | GLN | A | 330 | 76.569 | −66.941 | 106.388 | 1.00 | 108.05 | | O |
| ANISOU | 2392 | O | GLN | A | 330 | 11936 | 15082 | 14035 | −1421 | 4146 | −2714 | O |
| ATOM | 2393 | CB | GLN | A | 330 | 73.530 | −67.449 | 106.092 | 1.00 | 110.90 | | C |
| ANISOU | 2393 | CB | GLN | A | 330 | 11431 | 16451 | 14256 | −2026 | 4625 | −3775 | C |
| ATOM | 2394 | CG | GLN | A | 330 | 72.029 | −67.247 | 106.011 | 1.00 | 128.03 | | C |
| ANISOU | 2394 | CG | GLN | A | 330 | 13107 | 19289 | 16250 | −2178 | 4703 | −4227 | C |
| ATOM | 2395 | CD | GLN | A | 330 | 71.343 | −68.449 | 105.425 | 1.00 | 153.06 | | C |
| ANISOU | 2395 | CD | GLN | A | 330 | 15918 | 22696 | 19542 | −2761 | 5181 | −4930 | C |
| ATOM | 2396 | OE1 | GLN | A | 330 | 71.727 | −68.953 | 104.362 | 1.00 | 152.12 | | O |
| ANISOU | 2396 | OE1 | GLN | A | 330 | 15594 | 22760 | 19444 | −2952 | 5220 | −5202 | O |
| ATOM | 2397 | NE2 | GLN | A | 330 | 70.304 | −68.931 | 106.098 | 1.00 | 142.01 | | N |
| ANISOU | 2397 | NE2 | GLN | A | 330 | 14419 | 21305 | 18232 | −3086 | 5590 | −5280 | N |
| ATOM | 2398 | N | SER | A | 331 | 75.718 | −66.173 | 108.357 | 1.00 | 99.80 | | N |
| ANISOU | 2398 | N | SER | A | 331 | 11155 | 13865 | 12901 | −1204 | 4161 | −2446 | N |
| ATOM | 2399 | CA | SER | A | 331 | 76.880 | −66.382 | 109.233 | 1.00 | 96.22 | | C |
| ANISOU | 2399 | CA | SER | A | 331 | 11180 | 12844 | 12536 | −1023 | 4175 | −2024 | C |
| ATOM | 2400 | C | SER | A | 331 | 76.612 | −65.763 | 110.620 | 1.00 | 93.26 | | C |
| ANISOU | 2400 | C | SER | A | 331 | 11028 | 12344 | 12061 | −811 | 4122 | −1778 | C |
| ATOM | 2401 | O | SER | A | 331 | 75.793 | −64.860 | 110.766 | 1.00 | 91.18 | | O |
| ANISOU | 2401 | O | SER | A | 331 | 10582 | 12403 | 11661 | −722 | 3953 | −1846 | O |
| ATOM | 2402 | CB | SER | A | 331 | 78.158 | −65.799 | 108.618 | 1.00 | 96.22 | | C |
| ANISOU | 2402 | CB | SER | A | 331 | 11247 | 12819 | 12495 | −778 | 3793 | −1760 | C |
| ATOM | 2403 | OG | SER | A | 331 | 79.314 | −66.016 | 109.411 | 1.00 | 96.21 | | O |
| ANISOU | 2403 | OG | SER | A | 331 | 11630 | 12392 | 12535 | −595 | 3788 | −1400 | O |
| ATOM | 2404 | N | LEU | A | 332 | 77.304 | −66.275 | 111.624 | 1.00 | 85.89 | | N |
| ANISOU | 2404 | N | LEU | A | 332 | 10481 | 10978 | 11175 | −701 | 4290 | −1490 | N |
| ATOM | 2405 | CA | LEU | A | 332 | 77.251 | −65.782 | 112.981 | 1.00 | 84.15 | | C |
| ANISOU | 2405 | CA | LEU | A | 332 | 10492 | 10652 | 10828 | −475 | 4244 | −1243 | C |
| ATOM | 2406 | C | LEU | A | 332 | 78.533 | −64.973 | 113.207 | 1.00 | 87.68 | | C |
| ANISOU | 2406 | C | LEU | A | 332 | 11087 | 11074 | 11151 | −158 | 3842 | −929 | C |
| ATOM | 2407 | O | LEU | A | 332 | 79.605 | −65.411 | 112.787 | 1.00 | 87.15 | | O |
| ANISOU | 2407 | O | LEU | A | 332 | 11113 | 10861 | 11139 | −105 | 3808 | −800 | O |
| ATOM | 2408 | CB | LEU | A | 332 | 77.180 | −66.982 | 113.932 | 1.00 | 83.44 | | C |
| ANISOU | 2408 | CB | LEU | A | 332 | 10712 | 10165 | 10828 | −525 | 4759 | −1134 | C |
| ATOM | 2409 | CG | LEU | A | 332 | 76.062 | −66.997 | 114.969 | 1.00 | 85.89 | | C |
| ANISOU | 2409 | CG | LEU | A | 332 | 11077 | 10461 | 11097 | −576 | 5019 | −1195 | C |
| ATOM | 2410 | CD1 | LEU | A | 332 | 74.720 | −66.774 | 114.344 | 1.00 | 84.92 | | C |
| ANISOU | 2410 | CD1 | LEU | A | 332 | 10569 | 10678 | 11020 | −877 | 5067 | −1633 | C |
| ATOM | 2411 | CD2 | LEU | A | 332 | 76.052 | −68.305 | 115.677 | 1.00 | 87.13 | | C |
| ANISOU | 2411 | CD2 | LEU | A | 332 | 11555 | 10172 | 11379 | −626 | 5629 | −1079 | C |
| ATOM | 2412 | N | HIS | A | 333 | 78.418 | −63.771 | 113.812 | 1.00 | 83.64 | | N |
| ANISOU | 2412 | N | HIS | A | 333 | 10570 | 10722 | 10485 | 26 | 3564 | −851 | N |
| ATOM | 2413 | CA | HIS | A | 333 | 79.552 | −62.878 | 114.089 | 1.00 | 82.63 | | C |
| ANISOU | 2413 | CA | HIS | A | 333 | 10541 | 10608 | 10248 | 262 | 3226 | −656 | C |
| ATOM | 2414 | C | HIS | A | 333 | 79.620 | −62.533 | 115.541 | 1.00 | 83.75 | | C |
| ANISOU | 2414 | C | HIS | A | 333 | 10872 | 10730 | 10220 | 434 | 3221 | −517 | C |
| ATOM | 2415 | O | HIS | A | 333 | 78.581 | −62.319 | 116.168 | 1.00 | 82.52 | | O |
| ANISOU | 2415 | O | HIS | A | 333 | 10708 | 10628 | 10018 | 402 | 3340 | −591 | O |
| ATOM | 2416 | CB | HIS | A | 333 | 79.419 | −61.549 | 113.332 | 1.00 | 83.43 | | C |
| ANISOU | 2416 | CB | HIS | A | 333 | 10440 | 10928 | 10333 | 311 | 2935 | −748 | C |
| ATOM | 2417 | CG | HIS | A | 333 | 79.662 | −61.612 | 111.865 | 1.00 | 86.58 | | C |
| ANISOU | 2417 | CG | HIS | A | 333 | 10646 | 11423 | 10829 | 241 | 2847 | −832 | C |
| ATOM | 2418 | ND1 | HIS | A | 333 | 79.824 | −60.461 | 111.131 | 1.00 | 88.20 | | N |
| ANISOU | 2418 | ND1 | HIS | A | 333 | 10726 | 11775 | 11011 | 365 | 2628 | −828 | N |
| ATOM | 2419 | CD2 | HIS | A | 333 | 79.745 | −62.678 | 111.039 | 1.00 | 88.69 | | C |
| ANISOU | 2419 | CD2 | HIS | A | 333 | 10834 | 11657 | 11206 | 71 | 2993 | −925 | C |
| ATOM | 2420 | CE1 | HIS | A | 333 | 80.015 | −60.858 | 109.888 | 1.00 | 88.08 | | C |
| ANISOU | 2420 | CE1 | HIS | A | 333 | 10550 | 11856 | 11061 | 294 | 2608 | −896 | C |
| ATOM | 2421 | NE2 | HIS | A | 333 | 79.984 | −62.185 | 109.788 | 1.00 | 88.61 | | N |
| ANISOU | 2421 | NE2 | HIS | A | 333 | 10628 | 11830 | 11212 | 97 | 2811 | −981 | N |
| ATOM | 2422 | N | THR | A | 334 | 80.842 | −62.424 | 116.066 | 1.00 | 79.38 | | N |
| ANISOU | 2422 | N | THR | A | 334 | 10451 | 10164 | 9545 | 621 | 3071 | −342 | N |
| ATOM | 2423 | CA | THR | A | 334 | 81.051 | −62.023 | 117.444 | 1.00 | 79.40 | | C |
| ANISOU | 2423 | CA | THR | A | 334 | 10583 | 10264 | 9322 | 807 | 3024 | −247 | C |
| ATOM | 2424 | C | THR | A | 334 | 82.420 | −61.423 | 117.602 | 1.00 | 81.56 | | C |
| ANISOU | 2424 | C | THR | A | 334 | 10839 | 10685 | 9464 | 950 | 2738 | −208 | C |
| ATOM | 2425 | O | THR | A | 334 | 83.411 | −61.982 | 117.145 | 1.00 | 80.90 | | O |
| ANISOU | 2425 | O | THR | A | 334 | 10766 | 10575 | 9398 | 1012 | 2697 | −103 | O |
| ATOM | 2426 | CB | THR | A | 334 | 80.713 | −63.138 | 118.488 | 1.00 | 92.57 | | C |
| ANISOU | 2426 | CB | THR | A | 334 | 12465 | 11821 | 10886 | 919 | 3374 | −65 | C |
| ATOM | 2427 | OG1 | THR | A | 334 | 80.750 | −62.586 | 119.812 | 1.00 | 91.86 | | O |
| ANISOU | 2427 | OG1 | THR | A | 334 | 12454 | 11917 | 10531 | 1108 | 3302 | −8 | O |
| ATOM | 2428 | CG2 | THR | A | 334 | 81.603 | −64.403 | 118.366 | 1.00 | 92.23 | | C |
| ANISOU | 2428 | CG2 | THR | A | 334 | 12577 | 11618 | 10851 | 1055 | 3579 | 173 | C |
| ATOM | 2429 | N | THR | A | 335 | 82.461 | −60.263 | 118.231 | 1.00 | 77.33 | | N |
| ANISOU | 2429 | N | THR | A | 335 | 10259 | 10314 | 8807 | 979 | 2568 | −333 | N |
| ATOM | 2430 | CA | THR | A | 335 | 83.707 | −59.618 | 118.551 | 1.00 | 77.09 | | C |
| ANISOU | 2430 | CA | THR | A | 335 | 10173 | 10485 | 8633 | 1061 | 2340 | −396 | C |
| ATOM | 2431 | C | THR | A | 335 | 83.980 | −59.981 | 120.007 | 1.00 | 82.85 | | C |
| ANISOU | 2431 | C | THR | A | 335 | 10988 | 11462 | 9029 | 1283 | 2380 | −297 | C |
| ATOM | 2432 | O | THR | A | 335 | 83.096 | −59.837 | 120.858 | 1.00 | 81.53 | | O |
| ANISOU | 2432 | O | THR | A | 335 | 10892 | 11323 | 8763 | 1307 | 2498 | −310 | O |
| ATOM | 2433 | CB | THR | A | 335 | 83.640 | −58.150 | 118.227 | 1.00 | 79.97 | | C |
| ANISOU | 2433 | CB | THR | A | 335 | 10433 | 10849 | 9101 | 925 | 2212 | −640 | C |
| ATOM | 2434 | OG1 | THR | A | 335 | 83.112 | −58.020 | 116.903 | 1.00 | 76.39 | | O |
| ANISOU | 2434 | OG1 | THR | A | 335 | 9921 | 10200 | 8905 | 814 | 2244 | −643 | O |
| ATOM | 2435 | CG2 | THR | A | 335 | 84.998 | −57.475 | 118.344 | 1.00 | 78.57 | | C |
| ANISOU | 2435 | CG2 | THR | A | 335 | 10161 | 10850 | 8843 | 917 | 2028 | −796 | C |
| ATOM | 2436 | N | VAL | A | 336 | 85.179 | −60.560 | 120.247 | 1.00 | 81.79 | | N |
| ANISOU | 2436 | N | VAL | A | 336 | 10840 | 11536 | 8701 | 1485 | 2306 | −164 | N |
| ATOM | 2437 | CA | VAL | A | 336 | 85.693 | −61.075 | 121.524 | 1.00 | 82.46 | | C |
| ANISOU | 2437 | CA | VAL | A | 336 | 10971 | 11974 | 8385 | 1815 | 2340 | 0 | C |
| ATOM | 2438 | C | VAL | A | 336 | 86.999 | −60.376 | 121.944 | 1.00 | 90.33 | | C |
| ANISOU | 2438 | C | VAL | A | 336 | 11746 | 13467 | 9110 | 1896 | 2051 | −202 | C |
| ATOM | 2439 | O | VAL | A | 336 | 87.563 | −59.599 | 121.179 | 1.00 | 90.53 | | O |
| ANISOU | 2439 | O | VAL | A | 336 | 11617 | 13467 | 9311 | 1671 | 1872 | −450 | O |
| ATOM | 2440 | CB | VAL | A | 336 | 85.899 | −62.617 | 121.467 | 1.00 | 85.70 | | C |
| ANISOU | 2440 | CB | VAL | A | 336 | 11563 | 12243 | 8758 | 2083 | 2607 | 398 | C |
| ATOM | 2441 | CG1 | VAL | A | 336 | 84.610 | −63.350 | 121.109 | 1.00 | 85.47 | | C |
| ANISOU | 2441 | CG1 | VAL | A | 336 | 11725 | 11743 | 9007 | 1937 | 2966 | 506 | C |
| ATOM | 2442 | CG2 | VAL | A | 336 | 87.021 | −62.994 | 120.505 | 1.00 | 85.31 | | C |
| ANISOU | 2442 | CG2 | VAL | A | 336 | 11433 | 12175 | 8806 | 2109 | 2498 | 459 | C |
| ATOM | 2443 | N | ILE | A | 337 | 87.480 | −60.683 | 123.148 | 1.00 | 89.88 | | N |
| ANISOU | 2443 | N | ILE | A | 337 | 11654 | 13890 | 8605 | 2228 | 2037 | −105 | N |
| ATOM | 2444 | CA | ILE | A | 337 | 88.737 | −60.173 | 123.655 | 1.00 | 91.83 | | C |
| ANISOU | 2444 | CA | ILE | A | 337 | 11623 | 14759 | 8508 | 2335 | 1779 | −331 | C |
| ATOM | 2445 | C | ILE | A | 337 | 89.649 | −61.390 | 123.703 | 1.00 | 101.96 | | C |
| ANISOU | 2445 | C | ILE | A | 337 | 12919 | 16284 | 9537 | 2773 | 1824 | 52 | C |
| ATOM | 2446 | O | ILE | A | 337 | 89.435 | −62.303 | 124.509 | 1.00 | 102.24 | | O |
| ANISOU | 2446 | O | ILE | A | 337 | 13115 | 16450 | 9282 | 3186 | 2034 | 424 | O |
| ATOM | 2447 | CB | ILE | A | 337 | 88.583 | −59.395 | 124.993 | 1.00 | 94.83 | | C |
| ANISOU | 2447 | CB | ILE | A | 337 | 11873 | 15642 | 8516 | 2372 | 1704 | −606 | C |
| ATOM | 2448 | CG1 | ILE | A | 337 | 87.851 | −58.038 | 124.770 | 1.00 | 95.29 | | C |
| ANISOU | 2448 | CG1 | ILE | A | 337 | 11909 | 15408 | 8888 | 1909 | 1688 | −1029 | C |
| ATOM | 2449 | CG2 | ILE | A | 337 | 89.927 | −59.193 | 125.725 | 1.00 | 95.42 | | C |
| ANISOU | 2449 | CG2 | ILE | A | 337 | 11608 | 16558 | 8090 | 2585 | 1474 | −819 | C |
| ATOM | 2450 | CD1 | ILE | A | 337 | 88.615 | −56.932 | 123.925 | 1.00 | 102.17 | | C |
| ANISOU | 2450 | CD1 | ILE | A | 337 | 12574 | 16225 | 10021 | 1535 | 1539 | −1462 | C |
| ATOM | 2451 | N | ALA | A | 338 | 90.606 | −61.431 | 122.759 | 1.00 | 101.75 | | N |
| ANISOU | 2451 | N | ALA | A | 338 | 12759 | 16244 | 9657 | 2696 | 1683 | −1 | N |
| ATOM | 2452 | CA | ALA | A | 338 | 91.573 | −62.500 | 122.598 | 1.00 | 103.14 | | C |
| ANISOU | 2452 | CA | ALA | A | 338 | 12928 | 16615 | 9647 | 3091 | 1722 | 342 | C |
| ATOM | 2453 | C | ALA | A | 338 | 93.037 | −62.033 | 122.723 | 1.00 | 111.05 | | C |
| ANISOU | 2453 | C | ALA | A | 338 | 13539 | 18317 | 10339 | 3177 | 1410 | 75 | C |
| ATOM | 2454 | O | ALA | A | 338 | 93.344 | −60.845 | 122.555 | 1.00 | 111.03 | | O |
| ANISOU | 2454 | O | ALA | A | 338 | 13289 | 18468 | 10428 | 2789 | 1191 | −432 | O |
| ATOM | 2455 | CB | ALA | A | 338 | 91.362 | −63.166 | 121.255 | 1.00 | 103.93 | | C |
| ANISOU | 2455 | CB | ALA | A | 338 | 13224 | 16044 | 10221 | 2930 | 1885 | 541 | C |
| ATOM | 2456 | N | LEU | A | 339 | 93.936 | −63.021 | 122.978 | 1.00 | 109.64 | | N |
| ANISOU | 2456 | N | LEU | A | 339 | 13312 | 18540 | 9808 | 3700 | 1447 | 429 | N |
| ATOM | 2457 | CA | LEU | A | 339 | 95.391 | −62.927 | 123.122 | 1.00 | 109.80 | | C |
| ANISOU | 2457 | CA | LEU | A | 339 | 12942 | 19326 | 9450 | 3926 | 1192 | 283 | C |
| ATOM | 2458 | C | LEU | A | 339 | 95.788 | −61.879 | 124.168 | 1.00 | 115.37 | | C |
| ANISOU | 2458 | C | LEU | A | 339 | 13244 | 20875 | 9715 | 3858 | 929 | −234 | C |
| ATOM | 2459 | O | LEU | A | 339 | 95.228 | −61.887 | 125.269 | 1.00 | 116.16 | | O |
| ANISOU | 2459 | O | LEU | A | 339 | 13388 | 21271 | 9475 | 4081 | 998 | −169 | O |
| ATOM | 2460 | CB | LEU | A | 339 | 96.064 | −62.719 | 121.747 | 1.00 | 109.70 | | C |
| ANISOU | 2460 | CB | LEU | A | 339 | 12837 | 18989 | 9855 | 3584 | 1072 | 112 | C |
| ATOM | 2461 | CG | LEU | A | 339 | 95.742 | −63.801 | 120.684 | 1.00 | 114.18 | | C |
| ANISOU | 2461 | CG | LEU | A | 339 | 13763 | 18791 | 10829 | 3641 | 1341 | 572 | C |
| ATOM | 2462 | CD1 | LEU | A | 339 | 96.219 | −63.397 | 119.326 | 1.00 | 114.11 | | C |
| ANISOU | 2462 | CD1 | LEU | A | 339 | 13664 | 18442 | 11250 | 3232 | 1211 | 340 | C |
| ATOM | 2463 | CD2 | LEU | A | 339 | 96.291 | −65.178 | 121.070 | 1.00 | 116.55 | | C |
| ANISOU | 2463 | CD2 | LEU | A | 339 | 14176 | 19323 | 10786 | 4322 | 1561 | 1141 | C |
| ATOM | 2464 | N | GLU | A | 340 | 96.716 | −60.969 | 123.845 | 1.00 | 111.70 | | N |
| ANISOU | 2464 | N | GLU | A | 340 | 12385 | 20794 | 9263 | 3523 | 667 | −783 | N |
| ATOM | 2465 | CA | GLU | A | 340 | 97.093 | −59.945 | 124.804 | 1.00 | 111.65 | | C |
| ANISOU | 2465 | CA | GLU | A | 340 | 11964 | 21586 | 8872 | 3369 | 471 | −1384 | C |
| ATOM | 2466 | C | GLU | A | 340 | 96.045 | −58.801 | 124.809 | 1.00 | 115.80 | | C |
| ANISOU | 2466 | C | GLU | A | 340 | 12632 | 21576 | 9791 | 2785 | 549 | −1787 | C |
| ATOM | 2467 | O | GLU | A | 340 | 96.294 | −57.747 | 124.214 | 1.00 | 116.78 | | O |
| ANISOU | 2467 | O | GLU | A | 340 | 12613 | 21496 | 10260 | 2241 | 504 | −2307 | O |
| ATOM | 2468 | CB | GLU | A | 340 | 98.520 | −59.453 | 124.526 | 1.00 | 112.93 | | C |
| ANISOU | 2468 | CB | GLU | A | 340 | 11618 | 22408 | 8881 | 3221 | 232 | −1872 | C |
| ATOM | 2469 | N | GLY | A | 341 | 94.876 | −59.059 | 125.440 | 1.00 | 109.78 | | N |
| ANISOU | 2469 | N | GLY | A | 341 | 12177 | 20547 | 8990 | 2932 | 716 | −1506 | N |
| ATOM | 2470 | CA | GLY | A | 341 | 93.727 | −58.156 | 125.592 | 1.00 | 108.47 | | C |
| ANISOU | 2470 | CA | GLY | A | 341 | 12188 | 19898 | 9129 | 2514 | 829 | −1765 | C |
| ATOM | 2471 | C | GLY | A | 341 | 93.298 | −57.383 | 124.356 | 1.00 | 109.75 | | C |
| ANISOU | 2471 | C | GLY | A | 341 | 12506 | 19223 | 9970 | 1962 | 906 | −1965 | C |
| ATOM | 2472 | O | GLY | A | 341 | 93.041 | −56.174 | 124.430 | 1.00 | 108.84 | | O |
| ANISOU | 2472 | O | GLY | A | 341 | 12317 | 18995 | 10042 | 1535 | 941 | −2456 | O |
| ATOM | 2473 | N | LEU | A | 342 | 93.225 | −58.087 | 123.204 | 1.00 | 104.84 | | N |
| ANISOU | 2473 | N | LEU | A | 342 | 12106 | 18021 | 9707 | 1996 | 971 | −1576 | N |
| ATOM | 2474 | CA | LEU | A | 342 | 92.917 | −57.488 | 121.904 | 1.00 | 103.50 | | C |
| ANISOU | 2474 | CA | LEU | A | 342 | 12064 | 17137 | 10124 | 1573 | 1039 | −1690 | C |
| ATOM | 2475 | C | LEU | A | 342 | 91.553 | −57.857 | 121.302 | 1.00 | 104.86 | | C |
| ANISOU | 2475 | C | LEU | A | 342 | 12608 | 16567 | 10666 | 1531 | 1235 | −1337 | C |
| ATOM | 2476 | O | LEU | A | 342 | 91.219 | −59.049 | 121.234 | 1.00 | 106.05 | | O |
| ANISOU | 2476 | O | LEU | A | 342 | 12951 | 16567 | 10777 | 1820 | 1336 | −882 | O |
| ATOM | 2477 | CB | LEU | A | 342 | 94.034 | −57.821 | 120.901 | 1.00 | 103.19 | | C |
| ANISOU | 2477 | CB | LEU | A | 342 | 11894 | 17104 | 10208 | 1568 | 936 | −1652 | C |
| ATOM | 2478 | N | PRO | A | 343 | 90.802 | −56.857 | 120.766 | 1.00 | 96.30 | | N |
| ANISOU | 2478 | N | PRO | A | 343 | 11614 | 15016 | 9958 | 1175 | 1329 | −1554 | N |
| ATOM | 2479 | CA | PRO | A | 343 | 89.536 | −57.174 | 120.088 | 1.00 | 94.05 | | C |
| ANISOU | 2479 | CA | PRO | A | 343 | 11601 | 14141 | 9993 | 1139 | 1493 | −1267 | C |
| ATOM | 2480 | C | PRO | A | 343 | 89.733 | −57.984 | 118.783 | 1.00 | 92.50 | | C |
| ANISOU | 2480 | C | PRO | A | 343 | 11482 | 13611 | 10054 | 1163 | 1507 | −988 | C |
| ATOM | 2481 | O | PRO | A | 343 | 90.356 | −57.532 | 117.800 | 1.00 | 91.07 | | O |
| ANISOU | 2481 | O | PRO | A | 343 | 11215 | 13291 | 10097 | 991 | 1447 | −1119 | O |
| ATOM | 2482 | CB | PRO | A | 343 | 88.887 | −55.798 | 119.868 | 1.00 | 95.78 | | C |
| ANISOU | 2482 | CB | PRO | A | 343 | 11842 | 14072 | 10476 | 830 | 1591 | −1577 | C |
| ATOM | 2483 | CG | PRO | A | 343 | 89.685 | −54.845 | 120.700 | 1.00 | 100.61 | | C |
| ANISOU | 2483 | CG | PRO | A | 343 | 12238 | 15099 | 10891 | 689 | 1533 | −2040 | C |
| ATOM | 2484 | CD | PRO | A | 343 | 91.058 | −55.407 | 120.740 | 1.00 | 96.81 | | C |
| ANISOU | 2484 | CD | PRO | A | 343 | 11535 | 15080 | 10169 | 812 | 1347 | −2069 | C |
| ATOM | 2485 | N | LEU | A | 344 | 89.228 | −59.230 | 118.826 | 1.00 | 85.02 | | N |
| ANISOU | 2485 | N | LEU | A | 344 | 10702 | 12538 | 9063 | 1381 | 1629 | −615 | N |
| ATOM | 2486 | CA | LEU | A | 344 | 89.256 | −60.199 | 117.740 | 1.00 | 82.76 | | C |
| ANISOU | 2486 | CA | LEU | A | 344 | 10511 | 11938 | 8997 | 1407 | 1714 | −358 | C |
| ATOM | 2487 | C | LEU | A | 344 | 87.824 | −60.474 | 117.270 | 1.00 | 82.45 | | C |
| ANISOU | 2487 | C | LEU | A | 344 | 10632 | 11491 | 9203 | 1282 | 1917 | −262 | C |
| ATOM | 2488 | O | LEU | A | 344 | 86.949 | −60.727 | 118.095 | 1.00 | 81.72 | | O |
| ANISOU | 2488 | O | LEU | A | 344 | 10652 | 11395 | 9003 | 1356 | 2064 | −185 | O |
| ATOM | 2489 | CB | LEU | A | 344 | 89.933 | −61.519 | 118.206 | 1.00 | 82.57 | | C |
| ANISOU | 2489 | CB | LEU | A | 344 | 10546 | 12111 | 8718 | 1768 | 1789 | −32 | C |
| ATOM | 2490 | CG | LEU | A | 344 | 89.871 | −62.726 | 117.236 | 1.00 | 86.69 | | C |
| ANISOU | 2490 | CG | LEU | A | 344 | 11217 | 12256 | 9466 | 1809 | 1988 | 249 | C |
| ATOM | 2491 | CD1 | LEU | A | 344 | 90.922 | −62.635 | 116.180 | 1.00 | 87.01 | | C |
| ANISOU | 2491 | CD1 | LEU | A | 344 | 11122 | 12292 | 9648 | 1728 | 1828 | 183 | C |
| ATOM | 2492 | CD2 | LEU | A | 344 | 90.064 | −64.028 | 117.944 | 1.00 | 88.36 | | C |
| ANISOU | 2492 | CD2 | LEU | A | 344 | 11595 | 12530 | 9448 | 2202 | 2235 | 625 | C |
| ATOM | 2493 | N | GLU | A | 345 | 87.589 | −60.442 | 115.954 | 1.00 | 75.92 | | N |
| ANISOU | 2493 | N | GLU | A | 345 | 9788 | 10381 | 8678 | 1103 | 1932 | −283 | N |
| ATOM | 2494 | CA | GLU | A | 345 | 86.281 | −60.762 | 115.390 | 1.00 | 74.34 | | C |
| ANISOU | 2494 | CA | GLU | A | 345 | 9655 | 9916 | 8674 | 981 | 2114 | −248 | C |
| ATOM | 2495 | C | GLU | A | 345 | 86.277 | −62.233 | 114.986 | 1.00 | 78.39 | | C |
| ANISOU | 2495 | C | GLU | A | 345 | 10267 | 10260 | 9257 | 1030 | 2323 | −44 | C |
| ATOM | 2496 | O | GLU | A | 345 | 87.256 | −62.729 | 114.438 | 1.00 | 79.18 | | O |
| ANISOU | 2496 | O | GLU | A | 345 | 10351 | 10347 | 9389 | 1088 | 2280 | 44 | O |
| ATOM | 2497 | CB | GLU | A | 345 | 85.993 | −59.866 | 114.187 | 1.00 | 75.07 | | C |
| ANISOU | 2497 | CB | GLU | A | 345 | 9636 | 9893 | 8995 | 804 | 2035 | −400 | C |
| ATOM | 2498 | CG | GLU | A | 345 | 84.604 | −59.990 | 113.599 | 1.00 | 80.95 | | C |
| ANISOU | 2498 | CG | GLU | A | 345 | 10357 | 10528 | 9872 | 703 | 2181 | −426 | C |
| ATOM | 2499 | CD | GLU | A | 345 | 84.366 | −58.902 | 112.571 | 1.00 | 94.67 | | C |
| ANISOU | 2499 | CD | GLU | A | 345 | 11976 | 12251 | 11743 | 649 | 2105 | −531 | C |
| ATOM | 2500 | OE1 | GLU | A | 345 | 84.524 | −59.181 | 111.359 | 1.00 | 69.71 | | O |
| ANISOU | 2500 | OE1 | GLU | A | 345 | 8727 | 9060 | 8700 | 607 | 2089 | −515 | O |
| ATOM | 2501 | OE2 | GLU | A | 345 | 84.060 | −57.757 | 112.979 | 1.00 | 92.49 | | O |
| ANISOU | 2501 | OE2 | GLU | A | 345 | 11706 | 11993 | 11444 | 674 | 2094 | −618 | O |
| ATOM | 2502 | N | VAL | A | 346 | 85.208 | −62.939 | 115.302 | 1.00 | 74.61 | | N |
| ANISOU | 2502 | N | VAL | A | 346 | 9898 | 9640 | 8811 | 1001 | 2596 | 16 | N |
| ATOM | 2503 | CA | VAL | A | 346 | 85.052 | −64.345 | 114.946 | 1.00 | 75.01 | | C |
| ANISOU | 2503 | CA | VAL | A | 346 | 10068 | 9452 | 8980 | 990 | 2921 | 154 | C |
| ATOM | 2504 | C | VAL | A | 346 | 83.823 | −64.445 | 114.041 | 1.00 | 81.78 | | C |
| ANISOU | 2504 | C | VAL | A | 346 | 10825 | 10180 | 10069 | 693 | 3069 | −56 | C |
| ATOM | 2505 | O | VAL | A | 346 | 82.819 | −63.779 | 114.279 | 1.00 | 81.58 | | O |
| ANISOU | 2505 | O | VAL | A | 346 | 10724 | 10243 | 10030 | 604 | 3043 | −198 | O |
| ATOM | 2506 | CB | VAL | A | 346 | 84.987 | −65.301 | 116.179 | 1.00 | 78.88 | | C |
| ANISOU | 2506 | CB | VAL | A | 346 | 10792 | 9883 | 9295 | 1236 | 3239 | 410 | C |
| ATOM | 2507 | CG1 | VAL | A | 346 | 84.780 | −66.759 | 115.763 | 1.00 | 78.85 | | C |
| ANISOU | 2507 | CG1 | VAL | A | 346 | 10956 | 9525 | 9478 | 1196 | 3705 | 536 | C |
| ATOM | 2508 | CG2 | VAL | A | 346 | 86.239 | −65.180 | 117.041 | 1.00 | 78.58 | | C |
| ANISOU | 2508 | CG2 | VAL | A | 346 | 10778 | 10137 | 8941 | 1593 | 3059 | 606 | C |
| ATOM | 2509 | N | GLN | A | 347 | 83.930 | −65.238 | 112.982 | 1.00 | 81.09 | | N |
| ANISOU | 2509 | N | GLN | A | 347 | 10700 | 9942 | 10169 | 549 | 3216 | −102 | N |
| ATOM | 2510 | CA | GLN | A | 347 | 82.856 | −65.472 | 112.029 | 1.00 | 82.44 | | C |
| ANISOU | 2510 | CA | GLN | A | 347 | 10705 | 10100 | 10519 | 257 | 3368 | −362 | C |
| ATOM | 2511 | C | GLN | A | 347 | 82.631 | −66.982 | 111.970 | 1.00 | 91.52 | | C |
| ANISOU | 2511 | C | GLN | A | 347 | 11995 | 10958 | 11822 | 129 | 3863 | −362 | C |
| ATOM | 2512 | O | GLN | A | 347 | 83.560 | −67.748 | 111.736 | 1.00 | 90.73 | | O |
| ANISOU | 2512 | O | GLN | A | 347 | 12024 | 10668 | 11779 | 214 | 3989 | −205 | O |
| ATOM | 2513 | CB | GLN | A | 347 | 83.205 | −64.884 | 110.649 | 1.00 | 83.55 | | C |
| ANISOU | 2513 | CB | GLN | A | 347 | 10618 | 10392 | 10736 | 172 | 3095 | −497 | C |
| ATOM | 2514 | CG | GLN | A | 347 | 83.268 | −63.352 | 110.611 | 1.00 | 89.17 | | C |
| ANISOU | 2514 | CG | GLN | A | 347 | 11216 | 11318 | 11347 | 282 | 2735 | −513 | C |
| ATOM | 2515 | CD | GLN | A | 347 | 83.886 | −62.797 | 109.341 | 1.00 | 98.08 | | C |
| ANISOU | 2515 | CD | GLN | A | 347 | 12192 | 12538 | 12537 | 281 | 2517 | −552 | C |
| ATOM | 2516 | OE1 | GLN | A | 347 | 84.745 | −61.905 | 109.369 | 1.00 | 88.03 | | O |
| ANISOU | 2516 | OE1 | GLN | A | 347 | 10940 | 11279 | 11227 | 403 | 2296 | −475 | O |
| ATOM | 2517 | NE2 | GLN | A | 347 | 83.442 | −63.277 | 108.191 | 1.00 | 89.52 | | N |
| ANISOU | 2517 | NE2 | GLN | A | 347 | 10932 | 11541 | 11539 | 130 | 2603 | −706 | N |
| ATOM | 2518 | N | ILE | A | 348 | 81.425 | −67.413 | 112.267 | 1.00 | 93.23 | | N |
| ANISOU | 2518 | N | ILE | A | 348 | 12203 | 11112 | 12107 | −64 | 4195 | −532 | N |
| ATOM | 2519 | CA | ILE | A | 348 | 81.096 | −68.830 | 112.274 | 1.00 | 95.72 | | C |
| ANISOU | 2519 | CA | ILE | A | 348 | 12673 | 11085 | 12612 | −239 | 4787 | −586 | C |
| ATOM | 2520 | C | ILE | A | 348 | 80.104 | −69.084 | 111.144 | 1.00 | 104.86 | | C |
| ANISOU | 2520 | C | ILE | A | 348 | 13520 | 12377 | 13945 | −669 | 4939 | −1058 | C |
| ATOM | 2521 | O | ILE | A | 348 | 79.051 | −68.448 | 111.100 | 1.00 | 104.88 | | O |
| ANISOU | 2521 | O | ILE | A | 348 | 13280 | 12683 | 13886 | −803 | 4821 | −1303 | O |
| ATOM | 2522 | CB | ILE | A | 348 | 80.523 | −69.272 | 113.665 | 1.00 | 99.31 | | C |
| ANISOU | 2522 | CB | ILE | A | 348 | 13393 | 11334 | 13008 | −128 | 5173 | −421 | C |
| ATOM | 2523 | CG1 | ILE | A | 348 | 81.439 | −68.865 | 114.842 | 1.00 | 99.78 | | C |
| ANISOU | 2523 | CG1 | ILE | A | 348 | 13678 | 11442 | 12792 | 340 | 4954 | 9 | C |
| ATOM | 2524 | CG2 | ILE | A | 348 | 80.223 | −70.771 | 113.697 | 1.00 | 100.57 | | C |
| ANISOU | 2524 | CG2 | ILE | A | 348 | 13771 | 11038 | 13404 | −302 | 5915 | −456 | C |
| ATOM | 2525 | CD1 | ILE | A | 348 | 80.716 | −68.211 | 115.963 | 1.00 | 105.75 | | C |
| ANISOU | 2525 | CD1 | ILE | A | 348 | 14463 | 12351 | 13366 | 438 | 4883 | 38 | C |
| ATOM | 2526 | N | ARG | A | 349 | 80.438 | −70.003 | 110.234 | 1.00 | 105.39 | | N |
| ANISOU | 2526 | N | ARG | A | 349 | 13567 | 12271 | 14204 | −873 | 5208 | −1206 | N |
| ATOM | 2527 | CA | ARG | A | 349 | 79.546 | −70.402 | 109.140 | 1.00 | 107.38 | | C |
| ANISOU | 2527 | CA | ARG | A | 349 | 13484 | 12713 | 14603 | −1318 | 5411 | −1730 | C |
| ATOM | 2528 | C | ARG | A | 349 | 79.827 | −71.843 | 108.703 | 1.00 | 115.78 | | C |
| ANISOU | 2528 | C | ARG | A | 349 | 14697 | 13353 | 15941 | −1572 | 6018 | −1870 | C |
| ATOM | 2529 | O | ARG | A | 349 | 80.878 | −72.391 | 109.042 | 1.00 | 115.93 | | O |
| ANISOU | 2529 | O | ARG | A | 349 | 15062 | 12972 | 16013 | −1325 | 6176 | −1493 | O |
| ATOM | 2530 | CB | ARG | A | 349 | 79.610 | −69.416 | 107.948 | 1.00 | 108.49 | | C |
| ANISOU | 2530 | CB | ARG | A | 349 | 13234 | 13374 | 14613 | −1316 | 4863 | −1901 | C |
| ATOM | 2531 | CG | ARG | A | 349 | 80.875 | −69.504 | 107.096 | 1.00 | 120.66 | | C |
| ANISOU | 2531 | CG | ARG | A | 349 | 14814 | 14840 | 16191 | −1207 | 4666 | −1753 | C |
| ATOM | 2532 | CD | ARG | A | 349 | 80.818 | −68.573 | 105.900 | 1.00 | 133.47 | | C |
| ANISOU | 2532 | CD | ARG | A | 349 | 16060 | 16974 | 17679 | −1194 | 4213 | −1920 | C |
| ATOM | 2533 | NE | ARG | A | 349 | 81.090 | −67.185 | 106.271 | 1.00 | 141.87 | | N |
| ANISOU | 2533 | NE | ARG | A | 349 | 17138 | 18215 | 18549 | −849 | 3730 | −1633 | N |
| ATOM | 2534 | CZ | ARG | A | 349 | 81.619 | −66.278 | 105.458 | 1.00 | 153.86 | | C |
| ANISOU | 2534 | CZ | ARG | A | 349 | 18516 | 19977 | 19969 | −676 | 3345 | −1551 | C |
| ATOM | 2535 | NH1 | ARG | A | 349 | 81.839 | −65.046 | 105.887 | 1.00 | 144.49 | | N |
| ANISOU | 2535 | NH1 | ARG | A | 349 | 17384 | 18864 | 18653 | −398 | 3024 | −1319 | N |
| ATOM | 2536 | NH2 | ARG | A | 349 | 81.929 | −66.596 | 104.207 | 1.00 | 136.06 | | N |
| ANISOU | 2536 | NH2 | ARG | A | 349 | 16072 | 17875 | 17750 | −788 | 3325 | −1715 | N |
| ATOM | 2537 | N | THR | A | 350 | 78.891 | −72.452 | 107.955 | 1.00 | 114.65 | | N |
| ANISOU | 2537 | N | THR | A | 350 | 14274 | 13328 | 15959 | −2058 | 6385 | −2433 | N |
| ATOM | 2538 | CA | THR | A | 350 | 79.074 | −73.796 | 107.396 | 1.00 | 115.30 | | C |
| ANISOU | 2538 | CA | THR | A | 350 | 14454 | 13015 | 16339 | −2392 | 7027 | −2686 | C |
| ATOM | 2539 | C | THR | A | 350 | 79.708 | −73.698 | 105.994 | 1.00 | 121.40 | | C |
| ANISOU | 2539 | C | THR | A | 350 | 14960 | 14063 | 17102 | −2480 | 6722 | −2872 | C |
| ATOM | 2540 | O | THR | A | 350 | 79.993 | −72.585 | 105.541 | 1.00 | 121.03 | | O |
| ANISOU | 2540 | O | THR | A | 350 | 14689 | 14480 | 16818 | −2248 | 6044 | −2754 | O |
| ATOM | 2541 | CB | THR | A | 350 | 77.759 | −74.568 | 107.394 | 1.00 | 120.60 | | C |
| ANISOU | 2541 | CB | THR | A | 350 | 14958 | 13655 | 17209 | −2932 | 7673 | −3275 | C |
| ATOM | 2542 | OG1 | THR | A | 350 | 76.734 | −73.781 | 106.779 | 1.00 | 116.63 | | O |
| ANISOU | 2542 | OG1 | THR | A | 350 | 13890 | 13905 | 16518 | −3150 | 7289 | −3747 | O |
| ATOM | 2543 | CG2 | THR | A | 350 | 77.339 | −74.986 | 108.775 | 1.00 | 120.04 | | C |
| ANISOU | 2543 | CG2 | THR | A | 350 | 15273 | 13114 | 17225 | −2843 | 8169 | −3035 | C |
| ATOM | 2544 | N | ARG | A | 351 | 79.933 | −74.836 | 105.312 | 1.00 | 119.67 | | N |
| ANISOU | 2544 | N | ARG | A | 351 | 14782 | 13542 | 17147 | −2805 | 7258 | −3156 | N |
| ATOM | 2545 | CA | ARG | A | 351 | 80.510 | −74.818 | 103.965 | 1.00 | 120.77 | | C |
| ANISOU | 2545 | CA | ARG | A | 351 | 14662 | 13949 | 17275 | −2911 | 7011 | −3364 | C |
| ATOM | 2546 | C | ARG | A | 351 | 79.515 | −74.238 | 102.955 | 1.00 | 128.00 | | C |
| ANISOU | 2546 | C | ARG | A | 351 | 14949 | 15676 | 18009 | −3216 | 6702 | −3959 | C |
| ATOM | 2547 | O | ARG | A | 351 | 79.888 | −73.477 | 102.060 | 1.00 | 127.38 | | O |
| ANISOU | 2547 | O | ARG | A | 351 | 14598 | 16079 | 17721 | −3056 | 6145 | −3939 | O |
| ATOM | 2548 | CB | ARG | A | 351 | 80.955 | −76.232 | 103.543 | 1.00 | 120.78 | | C |
| ANISOU | 2548 | CB | ARG | A | 351 | 14879 | 13391 | 17619 | −3202 | 7741 | −3550 | C |
| ATOM | 2549 | CG | ARG | A | 351 | 82.464 | −76.385 | 103.355 | 1.00 | 126.28 | | C |
| ANISOU | 2549 | CG | ARG | A | 351 | 15889 | 13748 | 18344 | −2822 | 7596 | −3043 | C |
| ATOM | 2550 | CD | ARG | A | 351 | 83.206 | −76.619 | 104.656 | 1.00 | 131.29 | | C |
| ANISOU | 2550 | CD | ARG | A | 351 | 17074 | 13819 | 18992 | −2328 | 7777 | −2351 | C |
| ATOM | 2551 | NE | ARG | A | 351 | 84.654 | −76.617 | 104.461 | 1.00 | 140.85 | | N |
| ANISOU | 2551 | NE | ARG | A | 351 | 18492 | 14867 | 20157 | −1921 | 7536 | −1881 | N |
| ATOM | 2552 | CZ | ARG | A | 351 | 85.535 | −77.023 | 105.371 | 1.00 | 157.61 | | C |
| ANISOU | 2552 | CZ | ARG | A | 351 | 21060 | 16554 | 22272 | −1452 | 7748 | −1290 | C |
| ATOM | 2553 | NH1 | ARG | A | 351 | 86.833 | −76.991 | 105.104 | 1.00 | 150.94 | | N |
| ANISOU | 2553 | NH1 | ARG | A | 351 | 20323 | 15666 | 21360 | −1100 | 7499 | −925 | N |
| ATOM | 2554 | NH2 | ARG | A | 351 | 85.124 | −77.470 | 106.552 | 1.00 | 141.52 | | N |
| ANISOU | 2554 | NH2 | ARG | A | 351 | 19344 | 14162 | 20264 | −1303 | 8223 | −1056 | N |
| ATOM | 2555 | N | GLU | A | 352 | 78.246 | −74.583 | 103.134 | 1.00 | 127.61 | | N |
| ANISOU | 2555 | N | GLU | A | 352 | 14664 | 15807 | 18015 | −3618 | 7086 | −4470 | N |
| ATOM | 2556 | CA | GLU | A | 352 | 77.147 | −74.158 | 102.276 | 1.00 | 129.08 | | C |
| ANISOU | 2556 | CA | GLU | A | 352 | 14195 | 16858 | 17990 | −3910 | 6882 | −5097 | C |
| ATOM | 2557 | C | GLU | A | 352 | 76.898 | −72.652 | 102.372 | 1.00 | 135.61 | | C |
| ANISOU | 2557 | C | GLU | A | 352 | 14805 | 18287 | 18434 | −3460 | 6117 | −4797 | C |
| ATOM | 2558 | O | GLU | A | 352 | 76.777 | −71.990 | 101.336 | 1.00 | 135.47 | | O |
| ANISOU | 2558 | O | GLU | A | 352 | 14347 | 18974 | 18149 | −3376 | 5680 | −4972 | O |
| ATOM | 2559 | CB | GLU | A | 352 | 75.898 | −74.997 | 102.579 | 1.00 | 130.70 | | C |
| ANISOU | 2559 | CB | GLU | A | 352 | 14223 | 17056 | 18380 | −4477 | 7570 | −5739 | C |
| ATOM | 2560 | CG | GLU | A | 352 | 75.978 | −76.423 | 102.033 | 1.00 | 144.74 | | C |
| ANISOU | 2560 | CG | GLU | A | 352 | 16035 | 18437 | 20523 | −5048 | 8368 | −6275 | C |
| ATOM | 2561 | CD | GLU | A | 352 | 77.249 | −77.222 | 102.291 | 1.00 | 167.74 | | C |
| ANISOU | 2561 | CD | GLU | A | 352 | 19575 | 20418 | 23740 | −4883 | 8747 | −5795 | C |
| ATOM | 2562 | OE1 | GLU | A | 352 | 77.553 | −77.494 | 103.477 | 1.00 | 168.30 | | O |
| ANISOU | 2562 | OE1 | GLU | A | 352 | 20199 | 19784 | 23962 | −4621 | 9052 | −5267 | O |
| ATOM | 2563 | OE2 | GLU | A | 352 | 77.953 | −77.552 | 101.308 | 1.00 | 155.53 | | O |
| ANISOU | 2563 | OE2 | GLU | A | 352 | 17956 | 18892 | 22247 | −4969 | 8729 | −5922 | O |
| ATOM | 2564 | N | MET | A | 353 | 76.927 | −72.101 | 103.611 | 1.00 | 133.70 | | N |
| ANISOU | 2564 | N | MET | A | 353 | 14907 | 17734 | 18157 | −3121 | 5980 | −4297 | N |
| ATOM | 2565 | CA | MET | A | 353 | 76.782 | −70.670 | 103.907 | 1.00 | 134.09 | | C |
| ANISOU | 2565 | CA | MET | A | 353 | 14866 | 18181 | 17901 | −2675 | 5350 | −3951 | C |
| ATOM | 2566 | C | MET | A | 353 | 78.029 | −69.887 | 103.435 | 1.00 | 139.23 | | C |
| ANISOU | 2566 | C | MET | A | 353 | 15648 | 18819 | 18436 | −2252 | 4820 | −3482 | C |
| ATOM | 2567 | O | MET | A | 353 | 78.035 | −68.655 | 103.507 | 1.00 | 139.38 | | O |
| ANISOU | 2567 | O | MET | A | 353 | 15599 | 19134 | 18226 | −1885 | 4339 | −3208 | O |
| ATOM | 2568 | CB | MET | A | 353 | 76.516 | −70.439 | 105.413 | 1.00 | 136.42 | | C |
| ANISOU | 2568 | CB | MET | A | 353 | 15516 | 18094 | 18224 | −2493 | 5441 | −3606 | C |
| ATOM | 2569 | N | HIS | A | 354 | 79.072 | −70.599 | 102.945 | 1.00 | 135.92 | | N |
| ANISOU | 2569 | N | HIS | A | 354 | 15418 | 18035 | 18189 | −2312 | 4959 | −3406 | N |
| ATOM | 2570 | CA | HIS | A | 354 | 80.302 | −69.986 | 102.446 | 1.00 | 135.79 | | C |
| ANISOU | 2570 | CA | HIS | A | 354 | 15513 | 17985 | 18096 | −1969 | 4523 | −3017 | C |
| ATOM | 2571 | C | HIS | A | 354 | 80.187 | −69.652 | 100.957 | 1.00 | 140.90 | | C |
| ANISOU | 2571 | C | HIS | A | 354 | 15704 | 19259 | 18572 | −2024 | 4270 | −3320 | C |
| ATOM | 2572 | O | HIS | A | 354 | 80.116 | −70.555 | 100.116 | 1.00 | 140.31 | | O |
| ANISOU | 2572 | O | HIS | A | 354 | 15439 | 19277 | 18597 | −2373 | 4560 | −3741 | O |
| ATOM | 2573 | CB | HIS | A | 354 | 81.529 | −70.843 | 102.778 | 1.00 | 136.22 | | C |
| ANISOU | 2573 | CB | HIS | A | 354 | 16011 | 17362 | 18385 | −1921 | 4774 | −2714 | C |
| ATOM | 2574 | CG | HIS | A | 354 | 82.821 | −70.178 | 102.456 | 1.00 | 139.31 | | C |
| ANISOU | 2574 | CG | HIS | A | 354 | 16525 | 17707 | 18698 | −1567 | 4332 | −2307 | C |
| ATOM | 2575 | ND1 | HIS | A | 354 | 83.426 | −70.352 | 101.230 | 1.00 | 141.01 | | N |
| ANISOU | 2575 | ND1 | HIS | A | 354 | 16583 | 18061 | 18932 | −1633 | 4242 | −2430 | N |
| ATOM | 2576 | CD2 | HIS | A | 354 | 83.574 | −69.344 | 103.205 | 1.00 | 140.97 | | C |
| ANISOU | 2576 | CD2 | HIS | A | 354 | 16970 | 17779 | 18812 | −1181 | 3991 | −1838 | C |
| ATOM | 2577 | CE1 | HIS | A | 354 | 84.529 | −69.627 | 101.271 | 1.00 | 140.48 | | C |
| ANISOU | 2577 | CE1 | HIS | A | 354 | 16675 | 17903 | 18797 | −1285 | 3861 | −2014 | C |
| ATOM | 2578 | NE2 | HIS | A | 354 | 84.662 | −69.006 | 102.443 | 1.00 | 140.75 | | N |
| ANISOU | 2578 | NE2 | HIS | A | 354 | 16934 | 17781 | 18764 | −1024 | 3706 | −1676 | N |
| ATOM | 2579 | N | ARG | A | 355 | 80.130 | −68.329 | 100.665 | 1.00 | 138.52 | | N |
| ANISOU | 2579 | N | ARG | A | 355 | 15233 | 19397 | 18000 | −1658 | 3773 | −3106 | N |
| ATOM | 2580 | CA | ARG | A | 355 | 79.979 | −67.663 | 99.359 | 1.00 | 142.76 | | C |
| ANISOU | 2580 | CA | ARG | A | 355 | 15352 | 20616 | 18274 | −1517 | 3466 | −3243 | C |
| ATOM | 2581 | C | ARG | A | 355 | 78.638 | −68.038 | 98.655 | 1.00 | 157.13 | | C |
| ANISOU | 2581 | C | ARG | A | 355 | 16601 | 23187 | 19914 | −1816 | 3632 | −3879 | C |
| ATOM | 2582 | O | ARG | A | 355 | 77.798 | −67.131 | 98.461 | 1.00 | 158.52 | | O |
| ANISOU | 2582 | O | ARG | A | 355 | 16449 | 24001 | 19781 | −1572 | 3395 | −3907 | O |
| ATOM | 2583 | CB | ARG | A | 355 | 81.216 | −67.859 | 98.434 | 1.00 | 143.42 | | C |
| ANISOU | 2583 | CB | ARG | A | 355 | 15520 | 20550 | 18424 | −1461 | 3359 | −3113 | C |
| ATOM | 2584 | CG | ARG | A | 355 | 82.590 | −67.502 | 99.043 | 1.00 | 151.83 | | C |
| ANISOU | 2584 | CG | ARG | A | 355 | 17067 | 20985 | 19637 | −1183 | 3190 | −2555 | C |
| ATOM | 2585 | CD | ARG | A | 355 | 82.967 | −66.027 | 98.967 | 1.00 | 159.99 | | C |
| ANISOU | 2585 | CD | ARG | A | 355 | 18131 | 22166 | 20490 | −740 | 2772 | −2167 | C |
| ATOM | 2586 | NE | ARG | A | 355 | 84.383 | −65.817 | 99.297 | 1.00 | 167.37 | | N |
| ANISOU | 2586 | NE | ARG | A | 355 | 19429 | 22597 | 21568 | −568 | 2644 | −1774 | N |
| ATOM | 2587 | CZ | ARG | A | 355 | 84.962 | −64.628 | 99.450 | 1.00 | 177.79 | | C |
| ANISOU | 2587 | CZ | ARG | A | 355 | 20872 | 23859 | 22823 | −249 | 2373 | −1441 | C |
| ATOM | 2588 | NH1 | ARG | A | 355 | 86.253 | −64.545 | 99.744 | 1.00 | 161.35 | | N |
| ANISOU | 2588 | NH1 | ARG | A | 355 | 19059 | 21380 | 20868 | −156 | 2287 | −1178 | N |
| ATOM | 2589 | NH2 | ARG | A | 355 | 84.254 | −63.513 | 99.314 | 1.00 | 163.59 | | N |
| ANISOU | 2589 | NH2 | ARG | A | 355 | 18920 | 22406 | 20832 | −18 | 2230 | −1387 | N |
| ATOM | 2590 | OXT | ARG | A | 355 | 78.417 | −69.224 | 98.315 | 1.00 | 176.73 | | O |
| ANISOU | 2590 | OXT | ARG | A | 355 | 18950 | 25645 | 22555 | −2288 | 4029 | −4368 | O |
| TER | 2591 | | ARG | A | 355 | | | | | | | |
| HETATM | 2592 | MN | MN | B | 1 | 109.114 | −55.427 | 83.944 | 1.00 | 61.43 | | MN |
| HETATM | 2593 | O3P | AMP | C | 1 | 88.638 | −56.523 | 105.653 | 1.00 | 75.62 | | O |
| HETATM | 2594 | P | AMP | C | 1 | 89.260 | −56.847 | 104.304 | 1.00 | 73.07 | | P |
| HETATM | 2595 | O1P | AMP | C | 1 | 90.747 | −56.571 | 104.296 | 1.00 | 72.24 | | O |
| HETATM | 2596 | O2P | AMP | C | 1 | 88.417 | −56.102 | 103.236 | 1.00 | 71.24 | | O |
| HETATM | 2597 | O5′ | AMP | C | 1 | 89.033 | −58.420 | 104.107 | 1.00 | 73.07 | | O |
| HETATM | 2598 | C5′ | AMP | C | 1 | 88.259 | −58.998 | 103.042 | 1.00 | 75.71 | | C |
| HETATM | 2599 | C4′ | AMP | C | 1 | 88.956 | −60.283 | 102.673 | 1.00 | 79.99 | | C |
| HETATM | 2600 | C3′ | AMP | C | 1 | 88.132 | −61.285 | 101.867 | 1.00 | 82.47 | | C |
| HETATM | 2601 | O3′ | AMP | C | 1 | 88.826 | −61.854 | 100.759 | 1.00 | 79.93 | | O |
| HETATM | 2602 | C2′ | AMP | C | 1 | 87.636 | −62.281 | 102.924 | 1.00 | 86.23 | | C |
| HETATM | 2603 | O2′ | AMP | C | 1 | 87.564 | −63.593 | 102.368 | 1.00 | 89.14 | | O |
| HETATM | 2604 | C1′ | AMP | C | 1 | 88.691 | −62.178 | 104.044 | 1.00 | 85.11 | | C |
| HETATM | 2605 | O4′ | AMP | C | 1 | 89.391 | −60.964 | 103.869 | 1.00 | 81.18 | | O |
| HETATM | 2606 | N9 | AMP | C | 1 | 88.155 | −62.201 | 105.407 | 1.00 | 87.78 | | N |
| HETATM | 2607 | C4 | AMP | C | 1 | 88.523 | −63.042 | 106.434 | 1.00 | 90.06 | | C |
| HETATM | 2608 | C5 | AMP | C | 1 | 87.697 | −62.717 | 107.500 | 1.00 | 89.56 | | C |
| HETATM | 2609 | N7 | AMP | C | 1 | 86.845 | −61.677 | 107.161 | 1.00 | 89.98 | | N |
| HETATM | 2610 | C8 | AMP | C | 1 | 87.157 | −61.411 | 105.915 | 1.00 | 88.66 | | C |
| HETATM | 2611 | N3 | AMP | C | 1 | 89.468 | −63.997 | 106.417 | 1.00 | 91.84 | | N |
| HETATM | 2612 | C2 | AMP | C | 1 | 89.522 | −64.648 | 107.585 | 1.00 | 90.22 | | C |
| HETATM | 2613 | N1 | AMP | C | 1 | 88.788 | −64.455 | 108.686 | 1.00 | 88.48 | | N |
| HETATM | 2614 | C6 | AMP | C | 1 | 87.846 | −63.481 | 108.678 | 1.00 | 88.63 | | C |
| HETATM | 2615 | N6 | AMP | C | 1 | 87.080 | −63.331 | 109.762 | 1.00 | 87.90 | | N |
| HETATM | 2616 | O15 | GN3 | D | 501 | 79.349 | −61.410 | 101.022 | 1.00 | 162.60 | | O |
| HETATM | 2617 | C9 | GN3 | D | 501 | 80.107 | −61.435 | 101.990 | 1.00 | 161.84 | | C |
| HETATM | 2618 | N5 | GN3 | D | 501 | 81.325 | −62.086 | 101.913 | 1.00 | 161.96 | | N |
| HETATM | 2619 | C | GN3 | D | 501 | 82.236 | −62.171 | 102.935 | 1.00 | 161.08 | | C |
| HETATM | 2620 | N | GN3 | D | 501 | 83.363 | −62.841 | 102.684 | 1.00 | 160.60 | | N |
| HETATM | 2621 | C2 | GN3 | D | 501 | 79.890 | −60.839 | 103.287 | 1.00 | 159.63 | | C |
| HETATM | 2622 | N2 | GN3 | D | 501 | 78.826 | −60.108 | 103.788 | 1.00 | 157.44 | | N |
| HETATM | 2623 | C3 | GN3 | D | 501 | 79.177 | −59.827 | 105.023 | 1.00 | 155.58 | | C |
| HETATM | 2624 | N3 | GN3 | D | 501 | 80.407 | −60.329 | 105.364 | 1.00 | 153.89 | | N |
| HETATM | 2625 | C1 | GN3 | D | 501 | 80.871 | −60.983 | 104.247 | 1.00 | 158.48 | | C |
| HETATM | 2626 | N1 | GN3 | D | 501 | 82.050 | −61.627 | 104.138 | 1.00 | 160.35 | | N |
| HETATM | 2627 | C4 | GN3 | D | 501 | 81.093 | −60.200 | 106.658 | 1.00 | 146.33 | | C |
| HETATM | 2628 | C8 | GN3 | D | 501 | 82.564 | −59.799 | 106.529 | 1.00 | 142.62 | | C |
| HETATM | 2629 | O14 | GN3 | D | 501 | 83.297 | −60.208 | 107.678 | 1.00 | 141.84 | | O |
| HETATM | 2630 | C7 | GN3 | D | 501 | 82.430 | −58.281 | 106.483 | 1.00 | 139.08 | | C |
| HETATM | 2631 | O8 | GN3 | D | 501 | 83.576 | −57.582 | 107.016 | 1.00 | 136.43 | | O |
| HETATM | 2632 | P2 | GN3 | D | 501 | 84.774 | −56.778 | 106.405 | 1.00 | 131.16 | | P |
| HETATM | 2633 | O10 | GN3 | D | 501 | 84.686 | −56.520 | 104.976 | 1.00 | 130.03 | | O |
| HETATM | 2634 | N4 | GN3 | D | 501 | 84.842 | −55.397 | 107.268 | 1.00 | 128.70 | | N |
| HETATM | 2635 | P3 | GN3 | D | 501 | 84.536 | −53.818 | 107.005 | 1.00 | 128.27 | | P |
| HETATM | 2636 | O13 | GN3 | D | 501 | 83.459 | −53.443 | 106.102 | 1.00 | 126.88 | | O |
| HETATM | 2637 | O12 | GN3 | D | 501 | 85.888 | −53.191 | 106.451 | 1.00 | 129.84 | | O |
| HETATM | 2638 | O11 | GN3 | D | 501 | 84.335 | −53.039 | 108.363 | 1.00 | 128.61 | | O |
| HETATM | 2639 | O9 | GN3 | D | 501 | 86.075 | −57.637 | 106.666 | 1.00 | 130.50 | | O |
| HETATM | 2640 | C5 | GN3 | D | 501 | 81.306 | −58.012 | 107.481 | 1.00 | 136.33 | | C |
| HETATM | 2641 | O | GN3 | D | 501 | 80.455 | −59.178 | 107.403 | 1.00 | 141.25 | | O |
| HETATM | 2642 | C6 | GN3 | D | 501 | 80.492 | −56.774 | 107.204 | 1.00 | 128.58 | | C |
| HETATM | 2643 | O1 | GN3 | D | 501 | 79.367 | −56.740 | 108.108 | 1.00 | 120.09 | | O |
| HETATM | 2644 | P | GN3 | D | 501 | 79.346 | −56.753 | 109.689 | 1.00 | 108.50 | | P |
| HETATM | 2645 | O3 | GN3 | D | 501 | 78.100 | −57.359 | 110.204 | 1.00 | 105.96 | | O |
| HETATM | 2646 | O2 | GN3 | D | 501 | 80.630 | −57.443 | 110.140 | 1.00 | 107.38 | | O |
| HETATM | 2647 | O4 | GN3 | D | 501 | 79.466 | −55.224 | 110.115 | 1.00 | 102.99 | | O |
| HETATM | 2648 | P1 | GN3 | D | 501 | 80.382 | −54.462 | 111.174 | 1.00 | 99.23 | | P |
| HETATM | 2649 | O7 | GN3 | D | 501 | 80.544 | −53.030 | 110.791 | 1.00 | 99.96 | | O |
| HETATM | 2650 | O6 | GN3 | D | 501 | 81.709 | −55.226 | 111.209 | 1.00 | 96.36 | | O |
| HETATM | 2651 | O5 | GN3 | D | 501 | 79.698 | −54.643 | 112.532 | 1.00 | 99.36 | | O |
| HETATM | 2652 | OAE | TMO | E | 601 | 79.357 | −61.117 | 96.720 | 1.00 | 150.17 | | O |
| HETATM | 2653 | NAC | TMO | E | 601 | 80.425 | −61.908 | 97.147 | 1.00 | 150.46 | | N |
| HETATM | 2654 | CAB | TMO | E | 601 | 81.163 | −62.401 | 95.954 | 1.00 | 150.31 | | C |
| HETATM | 2655 | CAD | TMO | E | 601 | 81.321 | −61.083 | 97.999 | 1.00 | 150.27 | | C |
| HETATM | 2656 | CAA | TMO | E | 601 | 79.890 | −63.055 | 97.926 | 1.00 | 150.63 | | C |
| HETATM | 2657 | MG | MG | F | 1 | 86.020 | −58.246 | 104.515 | 1.00 | 39.63 | | MG2+ |
| HETATM | 2658 | MG | MG | F | 2 | 79.743 | −52.144 | 112.919 | 1.00 | 29.84 | | MG2+ |
| HETATM | 2659 | O | HOH | W | 1 | 76.608 | −54.573 | 112.370 | 1.00 | 42.40 | | O |
| HETATM | 2660 | O | HOH | W | 2 | 76.728 | −54.270 | 106.419 | 1.00 | 51.41 | | O |
| HETATM | 2661 | O | HOH | W | 3 | 89.165 | −65.911 | 103.309 | 1.00 | 27.20 | | O |
| HETATM | 2662 | O | HOH | W | 4 | 86.520 | −62.171 | 93.604 | 1.00 | 55.99 | | O |
| HETATM | 2663 | O | HOH | W | 5 | 81.522 | −58.665 | 100.487 | 1.00 | 30.69 | | O |
| HETATM | 2664 | O | HOH | W | 6 | 83.131 | −59.682 | 95.495 | 1.00 | 69.04 | | O |
| HETATM | 2665 | O | HOH | W | 7 | 84.570 | −60.672 | 101.803 | 1.00 | 47.82 | | O |
| HETATM | 2666 | O | HOH | W | 8 | 77.116 | −64.829 | 99.891 | 1.00 | 41.83 | | O |
| HETATM | 2667 | O | HOH | W | 9 | 79.391 | −54.022 | 105.910 | 1.00 | 33.21 | | O |
| HETATM | 2668 | O | HOH | W | 10 | 78.003 | −56.998 | 103.596 | 1.00 | 58.28 | | O |
| HETATM | 2669 | O | HOH | W | 11 | 88.163 | −55.811 | 94.913 | 1.00 | 43.19 | | O |
| HETATM | 2670 | O | HOH | W | 12 | 89.728 | −64.330 | 100.860 | 1.00 | 46.30 | | O |
| HETATM | 2671 | O | HOH | W | 13 | 77.312 | −58.830 | 96.952 | 1.00 | 45.40 | | O |
| HETATM | 2672 | O | HOH | W | 14 | 85.352 | −64.559 | 94.182 | 1.00 | 76.15 | | O |
| HETATM | 2673 | O | HOH | W | 15 | 95.547 | −66.735 | 107.036 | 1.00 | 51.15 | | O |
| HETATM | 2674 | O | HOH | W | 16 | 122.587 | −66.272 | 95.173 | 1.00 | 39.64 | | O |
| HETATM | 2675 | O | HOH | W | 17 | 75.646 | −52.022 | 108.034 | 1.00 | 79.10 | | O |
| HETATM | 2676 | O | HOH | W | 18 | 73.042 | −55.840 | 111.539 | 1.00 | 50.94 | | O |
| HETATM | 2677 | O | HOH | W | 19 | 71.212 | −58.458 | 111.837 | 1.00 | 42.97 | | O |
| HETATM | 2678 | O | HOH | W | 20 | 89.899 | −49.937 | 80.341 | 0.50 | 38.14 | | O |
| HETATM | 2679 | O | HOH | W | 21 | 83.212 | −54.778 | 104.108 | 1.00 | 30.00 | | O |
| END | | | | | | | | | | | | |

TABLE 3
Rel-ppGpp_complex_closed_form crystal structure atomic coordinates:

| | | | | | | | | | | |
|---|---|---|---|---|---|---|---|---|---|---|
| CRYST1 | 88.413 | 88.413 | 182.732 | 90.00 | 90.00 | 90.00 | P | 41 | 2 | 2 |

| | | | | | | | | | | | | |
|---|---|---|---|---|---|---|---|---|---|---|---|---|
| ATOM | 1 | O | LEU | A | 6 | 81.555 | 62.254 | 5.246 | 1.00 | 93.88 | | O |
| ANISOU | 1 | O | LEU | A | 6 | 12047 | 12030 | 11593 | −2785 | 3564 | 319 | O |
| ATOM | 2 | N | LEU | A | 6 | 80.867 | 61.066 | 2.791 | 1.00 | 90.86 | | N |
| ANISOU | 2 | N | LEU | A | 6 | 11605 | 11756 | 11162 | −2162 | 3432 | 619 | N |
| ATOM | 3 | CA | LEU | A | 6 | 82.283 | 61.247 | 3.208 | 1.00 | 90.87 | | C |
| ANISOU | 3 | CA | LEU | A | 6 | 11381 | 11985 | 11162 | −2568 | 3505 | 430 | C |
| ATOM | 4 | C | LEU | A | 6 | 82.336 | 61.437 | 4.728 | 1.00 | 93.13 | | C |
| ANISOU | 4 | C | LEU | A | 6 | 11632 | 12256 | 11498 | −2788 | 3466 | 279 | C |
| ATOM | 5 | O | GLY | A | 7 | 81.851 | 60.448 | 8.665 | 1.00 | 77.81 | | O |
| ANISOU | 5 | O | GLY | A | 7 | 9112 | 10741 | 9713 | −2954 | 2915 | −123 | O |
| ATOM | 6 | N | GLY | A | 7 | 83.227 | 60.706 | 5.403 | 1.00 | 85.87 | | N |
| ANISOU | 6 | N | GLY | A | 7 | 10325 | 11697 | 10606 | −2939 | 3311 | 110 | N |
| ATOM | 7 | CA | GLY | A | 7 | 83.364 | 60.803 | 6.869 | 1.00 | 82.86 | | C |
| ANISOU | 7 | CA | GLY | A | 7 | 9835 | 11391 | 10258 | −3143 | 3237 | −56 | C |
| ATOM | 8 | C | GLY | A | 7 | 82.279 | 60.016 | 7.581 | 1.00 | 78.55 | | C |
| ANISOU | 8 | C | GLY | A | 7 | 9201 | 10863 | 9780 | −2838 | 2962 | −22 | C |
| ATOM | 9 | N | LEU | A | 8 | 81.848 | 58.902 | 6.984 | 1.00 | 68.49 | | N |
| ANISOU | 9 | N | LEU | A | 8 | 7858 | 9636 | 8529 | −2458 | 2788 | 117 | N |
| ATOM | 10 | CA | LEU | A | 8 | 80.802 | 58.054 | 7.570 | 1.00 | 64.66 | | C |
| ANISOU | 10 | CA | LEU | A | 8 | 7300 | 9168 | 8099 | −2156 | 2537 | 163 | C |
| ATOM | 11 | C | LEU | A | 8 | 81.486 | 57.109 | 8.532 | 1.00 | 63.86 | | C |
| ANISOU | 11 | C | LEU | A | 8 | 6854 | 9400 | 8009 | −2202 | 2329 | 15 | C |
| ATOM | 12 | O | LEU | A | 8 | 81.036 | 56.913 | 9.613 | 1.00 | 63.40 | | O |
| ANISOU | 12 | O | LEU | A | 8 | 6778 | 9324 | 7988 | −2128 | 2188 | −19 | O |
| ATOM | 13 | CB | LEU | A | 8 | 80.202 | 57.268 | 6.420 | 1.00 | 63.80 | | C |
| ANISOU | 13 | CB | LEU | A | 8 | 7189 | 9061 | 7991 | −1792 | 2424 | 325 | C |
| ATOM | 14 | CG | LEU | A | 8 | 79.448 | 58.118 | 5.425 | 1.00 | 67.39 | | C |
| ANISOU | 14 | CG | LEU | A | 8 | 7954 | 9239 | 8412 | −1667 | 2594 | 486 | C |
| ATOM | 15 | CD1 | LEU | A | 8 | 78.652 | 57.245 | 4.519 | 1.00 | 67.65 | | C |
| ANISOU | 15 | CD1 | LEU | A | 8 | 7943 | 9376 | 8386 | −1710 | 2705 | 521 | C |
| ATOM | 16 | CD2 | LEU | A | 8 | 78.510 | 59.031 | 6.155 | 1.00 | 68.40 | | C |
| ANISOU | 16 | CD2 | LEU | A | 8 | 8133 | 9295 | 8562 | −1297 | 2435 | 617 | C |
| ATOM | 17 | N | TRP | A | 9 | 82.572 | 56.545 | 8.099 | 1.00 | 56.53 | | N |
| ANISOU | 17 | N | TRP | A | 9 | 5655 | 8787 | 7035 | −2297 | 2308 | −68 | N |
| ATOM | 18 | CA | TRP | A | 9 | 83.329 | 55.615 | 8.921 | 1.00 | 54.03 | | C |
| ANISOU | 18 | CA | TRP | A | 9 | 5002 | 8827 | 6699 | −2299 | 2109 | −208 | C |
| ATOM | 19 | C | TRP | A | 9 | 83.633 | 56.173 | 10.314 | 1.00 | 60.01 | | C |
| ANISOU | 19 | C | TRP | A | 9 | 5728 | 9615 | 7458 | −2556 | 2114 | −371 | C |
| ATOM | 20 | O | TRP | A | 9 | 83.351 | 55.491 | 11.308 | 1.00 | 60.22 | | O |
| ANISOU | 20 | O | TRP | A | 9 | 5657 | 9728 | 7498 | −2425 | 1918 | −413 | O |
| ATOM | 21 | CB | TRP | A | 9 | 84.593 | 55.101 | 8.232 | 1.00 | 50.51 | | C |
| ANISOU | 21 | CB | TRP | A | 9 | 4272 | 8734 | 6187 | −2350 | 2108 | −276 | C |
| ATOM | 22 | CG | TRP | A | 9 | 85.317 | 54.130 | 9.108 | 1.00 | 49.82 | | C |
| ANISOU | 22 | CG | TRP | A | 9 | 3855 | 9018 | 6057 | −2299 | 1895 | −416 | C |
| ATOM | 23 | CD1 | TRP | A | 9 | 86.433 | 54.378 | 9.850 | 1.00 | 52.34 | | C |
| ANISOU | 23 | CD1 | TRP | A | 9 | 3939 | 9629 | 6320 | −2565 | 1908 | −613 | C |
| ATOM | 24 | CD2 | TRP | A | 9 | 84.843 | 52.844 | 9.517 | 1.00 | 49.24 | | C |
| ANISOU | 24 | CD2 | TRP | A | 9 | 3693 | 9033 | 5984 | −1965 | 1640 | −376 | C |
| ATOM | 25 | CE2 | TRP | A | 9 | 85.776 | 52.328 | 10.443 | 1.00 | 53.03 | | C |
| ANISOU | 25 | CE2 | TRP | A | 9 | 3888 | 9866 | 6395 | −2012 | 1507 | −543 | C |
| ATOM | 26 | CE3 | TRP | A | 9 | 83.737 | 52.050 | 9.160 | 1.00 | 50.32 | | C |
| ANISOU | 26 | CE3 | TRP | A | 9 | 3967 | 8996 | 6158 | −1636 | 1515 | −221 | C |
| ATOM | 27 | NE1 | TRP | A | 9 | 86.750 | 53.279 | 10.608 | 1.00 | 51.47 | | N |
| ANISOU | 27 | NE1 | TRP | A | 9 | 3584 | 9810 | 6164 | −2377 | 1668 | −690 | N |
| ATOM | 28 | CZ2 | TRP | A | 9 | 85.640 | 51.057 | 11.017 | 1.00 | 52.31 | | C |
| ANISOU | 28 | CZ2 | TRP | A | 9 | 3685 | 9925 | 6267 | −1721 | 1265 | −545 | C |
| ATOM | 29 | CZ3 | TRP | A | 9 | 83.606 | 50.789 | 9.724 | 1.00 | 51.68 | | C |
| ANISOU | 29 | CZ3 | TRP | A | 9 | 4020 | 9315 | 6301 | −1389 | 1283 | −234 | C |
| ATOM | 30 | CH2 | TRP | A | 9 | 84.546 | 50.306 | 10.643 | 1.00 | 52.34 | | C |
| ANISOU | 30 | CH2 | TRP | A | 9 | 3859 | 9715 | 6312 | −1423 | 1165 | −388 | C |
| ATOM | 31 | N | ASN | A | 10 | 84.125 | 57.427 | 10.391 | 1.00 | 56.21 | | N |
| ANISOU | 31 | N | ASN | A | 10 | 5363 | 9037 | 6956 | −2920 | 2347 | −459 | N |
| ATOM | 32 | CA | ASN | A | 10 | 84.454 | 58.088 | 11.662 | 1.00 | 55.85 | | C |
| ANISOU | 32 | CA | ASN | A | 10 | 5316 | 9005 | 6901 | −3213 | 2381 | −629 | C |
| ATOM | 33 | C | ASN | A | 10 | 83.247 | 58.293 | 12.617 | 1.00 | 59.63 | | C |
| ANISOU | 33 | C | ASN | A | 10 | 6027 | 9195 | 7435 | −3089 | 2318 | −576 | C |
| ATOM | 34 | O | ASN | A | 10 | 83.442 | 58.541 | 13.811 | 1.00 | 60.36 | | O |
| ANISOU | 34 | O | ASN | A | 10 | 6080 | 9338 | 7517 | −3256 | 2279 | −716 | O |
| ATOM | 35 | CB | ASN | A | 10 | 85.175 | 59.392 | 11.406 | 1.00 | 56.02 | | C |
| ANISOU | 35 | CB | ASN | A | 10 | 5466 | 8939 | 6880 | −3641 | 2671 | −724 | C |
| ATOM | 36 | CG | ASN | A | 10 | 86.331 | 59.199 | 10.478 | 1.00 | 92.44 | | C |
| ANISOU | 36 | CG | ASN | A | 10 | 9845 | 13844 | 11433 | −3783 | 2752 | −781 | C |
| ATOM | 37 | ND2 | ASN | A | 10 | 87.472 | 58.808 | 11.035 | 1.00 | 82.74 | | N |
| ANISOU | 37 | ND2 | ASN | A | 10 | 8248 | 13043 | 10147 | −3963 | 2667 | −980 | N |
| ATOM | 38 | OD1 | ASN | A | 10 | 86.202 | 59.362 | 9.255 | 1.00 | 98.11 | | O |
| ANISOU | 38 | OD1 | ASN | A | 10 | 10703 | 14428 | 12148 | −3714 | 2887 | −647 | O |
| ATOM | 39 | N | ARG | A | 11 | 82.020 | 58.168 | 12.094 | 1.00 | 54.12 | | N |
| ANISOU | 39 | N | ARG | A | 11 | 5555 | 8220 | 6786 | −2795 | 2307 | −384 | N |
| ATOM | 40 | CA | ARG | A | 11 | 80.776 | 58.298 | 12.842 | 1.00 | 52.83 | | C |
| ANISOU | 40 | CA | ARG | A | 11 | 5602 | 7800 | 6673 | −2631 | 2253 | −314 | C |
| ATOM | 41 | C | ARG | A | 11 | 80.415 | 56.913 | 13.413 | 1.00 | 55.60 | | C |
| ANISOU | 41 | C | ARG | A | 11 | 5737 | 8344 | 7044 | −2351 | 1970 | −303 | C |
| ATOM | 42 | O | ARG | A | 11 | 80.023 | 56.796 | 14.585 | 1.00 | 54.47 | | O |
| ANISOU | 42 | O | ARG | A | 11 | 5602 | 8179 | 6916 | −2336 | 1871 | −359 | O |
| ATOM | 43 | CB | ARG | A | 11 | 79.713 | 58.838 | 11.878 | 1.00 | 52.74 | | C |
| ANISOU | 43 | CB | ARG | A | 11 | 5906 | 7453 | 6680 | −2449 | 2388 | −123 | C |
| ATOM | 44 | CG | ARG | A | 11 | 78.281 | 58.843 | 12.341 | 1.00 | 66.25 | | C |
| ANISOU | 44 | CG | ARG | A | 11 | 7812 | 8927 | 8433 | −2203 | 2329 | −19 | C |
| ATOM | 45 | CD | ARG | A | 11 | 77.395 | 59.053 | 11.133 | 1.00 | 86.75 | | C |
| ANISOU | 45 | CD | ARG | A | 11 | 10612 | 11324 | 11025 | −1952 | 2406 | 169 | C |
| ATOM | 46 | NE | ARG | A | 11 | 75.975 | 59.013 | 11.471 | 1.00 | 105.50 | | N |
| ANISOU | 46 | NE | ARG | A | 11 | 13130 | 13525 | 13429 | −1685 | 2338 | 268 | N |
| ATOM | 47 | CZ | ARG | A | 11 | 74.995 | 58.907 | 10.579 | 1.00 | 125.25 | | C |
| ANISOU | 47 | CZ | ARG | A | 11 | 15735 | 15931 | 15925 | −1391 | 2326 | 424 | C |
| ATOM | 48 | NH1 | ARG | A | 11 | 73.729 | 58.875 | 10.978 | 1.00 | 114.62 | | N |
| ANISOU | 48 | NH1 | ARG | A | 11 | 14482 | 14471 | 14596 | −1169 | 2264 | 491 | N |
| ATOM | 49 | NH2 | ARG | A | 11 | 75.273 | 58.792 | 9.283 | 1.00 | 111.67 | | N |
| ANISOU | 49 | NH2 | ARG | A | 11 | 14004 | 14252 | 14171 | −1314 | 2369 | 507 | N |
| ATOM | 50 | N | LEU | A | 12 | 80.606 | 55.863 | 12.585 | 1.00 | 51.94 | | N |
| ANISOU | 50 | N | LEU | A | 12 | 5097 | 8068 | 6570 | −2143 | 1850 | −238 | N |
| ATOM | 51 | CA | LEU | A | 12 | 80.318 | 54.471 | 12.937 | 1.00 | 51.25 | | C |
| ANISOU | 51 | CA | LEU | A | 12 | 4840 | 8148 | 6483 | −1875 | 1600 | −221 | C |
| ATOM | 52 | C | LEU | A | 12 | 81.382 | 53.841 | 13.850 | 1.00 | 55.44 | | C |
| ANISOU | 52 | C | LEU | A | 12 | 5097 | 9012 | 6957 | −1962 | 1461 | −389 | C |
| ATOM | 53 | O | LEU | A | 12 | 81.004 | 53.225 | 14.844 | 1.00 | 55.30 | | O |
| ANISOU | 53 | O | LEU | A | 12 | 5053 | 9022 | 6937 | −1853 | 1309 | −420 | O |
| ATOM | 54 | CB | LEU | A | 12 | 80.074 | 53.619 | 11.670 | 1.00 | 50.63 | | C |
| ANISOU | 54 | CB | LEU | A | 12 | 4716 | 8121 | 6402 | −1614 | 1533 | −91 | C |
| ATOM | 55 | CG | LEU | A | 12 | 79.913 | 52.099 | 11.837 | 1.00 | 53.61 | | C |
| ANISOU | 55 | CG | LEU | A | 12 | 4938 | 8673 | 6759 | −1351 | 1294 | −78 | C |
| ATOM | 56 | CD1 | LEU | A | 12 | 78.604 | 51.741 | 12.504 | 1.00 | 52.87 | | C |
| ANISOU | 56 | CD1 | LEU | A | 12 | 4984 | 8393 | 6709 | −1187 | 1200 | −12 | C |
| ATOM | 57 | CD2 | LEU | A | 12 | 80.009 | 51.410 | 10.501 | 1.00 | 55.00 | | C |
| ANISOU | 57 | CD2 | LEU | A | 12 | 5052 | 8938 | 6908 | −1165 | 1259 | 13 | C |
| ATOM | 58 | N | GLU | A | 13 | 82.694 | 54.003 | 13.527 | 1.00 | 51.22 | | N |
| ANISOU | 58 | N | GLU | A | 13 | 4358 | 8740 | 6362 | −2152 | 1517 | −502 | N |
| ATOM | 59 | CA | GLU | A | 13 | 83.821 | 53.454 | 14.296 | 1.00 | 50.49 | | C |
| ANISOU | 59 | CA | GLU | A | 13 | 3968 | 9026 | 6188 | −2223 | 1389 | −674 | C |
| ATOM | 60 | C | GLU | A | 13 | 83.646 | 53.553 | 15.848 | 1.00 | 55.14 | | C |
| ANISOU | 60 | C | GLU | A | 13 | 4567 | 9617 | 6765 | −2297 | 1299 | −787 | C |
| ATOM | 61 | O | GLU | A | 13 | 83.830 | 52.512 | 16.490 | 1.00 | 55.76 | | O |
| ANISOU | 61 | O | GLU | A | 13 | 4490 | 9908 | 6787 | −2108 | 1097 | −829 | O |
| ATOM | 62 | CB | GLU | A | 13 | 85.159 | 54.064 | 13.842 | 1.00 | 51.61 | | C |
| ANISOU | 62 | CB | GLU | A | 13 | 3927 | 9408 | 6272 | −2519 | 1531 | −803 | C |
| ATOM | 63 | CG | GLU | A | 13 | 86.403 | 53.413 | 14.439 | 1.00 | 65.17 | | C |
| ANISOU | 63 | CG | GLU | A | 13 | 5280 | 11598 | 7882 | −2545 | 1389 | −981 | C |
| ATOM | 64 | CD | GLU | A | 13 | 86.761 | 53.777 | 15.874 | 1.00 | 98.95 | | C |
| ANISOU | 64 | CD | GLU | A | 13 | 9475 | 16007 | 12116 | −2745 | 1338 | −1164 | C |
| ATOM | 65 | OE1 | GLU | A | 13 | 87.092 | 54.960 | 16.128 | 1.00 | 99.85 | | O |
| ANISOU | 65 | OE1 | GLU | A | 13 | 9645 | 16060 | 12233 | −3117 | 1515 | −1274 | O |
| ATOM | 66 | OE2 | GLU | A | 13 | 86.711 | 52.875 | 16.744 | 1.00 | 95.49 | | O |
| ANISOU | 66 | OE2 | GLU | A | 13 | 8930 | 15725 | 11627 | −2531 | 1125 | −1200 | O |
| ATOM | 67 | N | PRO | A | 14 | 83.299 | 54.728 | 16.477 | 1.00 | 50.46 | | N |
| ANISOU | 67 | N | PRO | A | 14 | 4168 | 8795 | 6208 | −2545 | 1440 | −836 | N |
| ATOM | 68 | CA | PRO | A | 14 | 83.177 | 54.775 | 17.953 | 1.00 | 49.38 | | C |
| ANISOU | 68 | CA | PRO | A | 14 | 4033 | 8680 | 6048 | −2604 | 1347 | −948 | C |
| ATOM | 69 | C | PRO | A | 14 | 82.131 | 53.851 | 18.552 | 1.00 | 49.24 | | C |
| ANISOU | 69 | C | PRO | A | 14 | 4095 | 8559 | 6054 | −2286 | 1169 | −851 | C |
| ATOM | 70 | O | PRO | A | 14 | 82.345 | 53.341 | 19.657 | 1.00 | 48.58 | | O |
| ANISOU | 70 | O | PRO | A | 14 | 3909 | 8640 | 5911 | −2240 | 1020 | −947 | O |
| ATOM | 71 | CB | PRO | A | 14 | 82.850 | 56.239 | 18.236 | 1.00 | 51.58 | | C |
| ANISOU | 71 | CB | PRO | A | 14 | 4577 | 8653 | 6368 | −2889 | 1563 | −976 | C |
| ATOM | 72 | CG | PRO | A | 14 | 83.373 | 56.970 | 17.034 | 1.00 | 56.64 | | C |
| ANISOU | 72 | CG | PRO | A | 14 | 5254 | 9254 | 7011 | −3083 | 1772 | −957 | C |
| ATOM | 73 | CD | PRO | A | 14 | 83.055 | 56.066 | 15.903 | 1.00 | 52.11 | | C |
| ANISOU | 73 | CD | PRO | A | 14 | 4626 | 8711 | 6463 | −2777 | 1698 | −796 | C |
| ATOM | 74 | N | ALA | A | 15 | 81.033 | 53.609 | 17.808 | 1.00 | 43.24 | | N |
| ANISOU | 74 | N | ALA | A | 15 | 3515 | 7547 | 5368 | −2072 | 1187 | −667 | N |
| ATOM | 75 | CA | ALA | A | 15 | 79.957 | 52.694 | 18.188 | 1.00 | 42.60 | | C |
| ANISOU | 75 | CA | ALA | A | 15 | 3517 | 7358 | 5311 | −1788 | 1043 | −565 | C |
| ATOM | 76 | C | ALA | A | 15 | 80.416 | 51.219 | 18.249 | 1.00 | 46.91 | | C |
| ANISOU | 76 | C | ALA | A | 15 | 3867 | 8173 | 5783 | −1555 | 835 | −576 | C |
| ATOM | 77 | O | ALA | A | 15 | 79.785 | 50.400 | 18.924 | 1.00 | 45.18 | | O |
| ANISOU | 77 | O | ALA | A | 15 | 3701 | 7914 | 5552 | −1370 | 705 | −542 | O |
| ATOM | 78 | CB | ALA | A | 15 | 78.788 | 52.836 | 17.223 | 1.00 | 43.00 | | C |
| ANISOU | 78 | CB | ALA | A | 15 | 3768 | 7129 | 5441 | −1642 | 1124 | −387 | C |
| ATOM | 79 | N | LEU | A | 16 | 81.519 | 50.897 | 17.544 | 1.00 | 45.63 | | N |
| ANISOU | 79 | N | LEU | A | 16 | 3495 | 8280 | 5561 | −1563 | 817 | −623 | N |
| ATOM | 80 | CA | LEU | A | 16 | 82.094 | 49.551 | 17.434 | 1.00 | 45.86 | | C |
| ANISOU | 80 | CA | LEU | A | 16 | 3348 | 8578 | 5499 | −1322 | 639 | −632 | C |
| ATOM | 81 | CB | LEU | A | 16 | 82.760 | 49.354 | 16.058 | 1.00 | 45.09 | | C |
| ANISOU | 81 | CB | LEU | A | 16 | 3124 | 8625 | 5385 | −1281 | 686 | −593 | C |
| ATOM | 82 | CG | LEU | A | 16 | 81.897 | 49.595 | 14.821 | 1.00 | 49.06 | | C |
| ANISOU | 82 | CG | LEU | A | 16 | 3800 | 8866 | 5976 | −1222 | 794 | −430 | C |
| ATOM | 83 | CD1 | LEU | A | 16 | 82.763 | 49.825 | 13.605 | 1.00 | 48.79 | | C |
| ANISOU | 83 | CD1 | LEU | A | 16 | 3636 | 8986 | 5917 | −1283 | 889 | −429 | C |
| ATOM | 84 | CD2 | LEU | A | 16 | 80.914 | 48.452 | 14.591 | 1.00 | 50.60 | | C |
| ANISOU | 84 | CD2 | LEU | A | 16 | 4110 | 8936 | 6181 | −923 | 667 | −303 | C |
| ATOM | 85 | C | LEU | A | 16 | 83.070 | 49.184 | 18.561 | 1.00 | 52.59 | | C |
| ANISOU | 85 | C | LEU | A | 16 | 4005 | 9745 | 6232 | −1334 | 509 | −797 | C |
| ATOM | 86 | O | LEU | A | 16 | 83.600 | 48.067 | 18.561 | 1.00 | 53.96 | | O |
| ANISOU | 86 | O | LEU | A | 16 | 4053 | 10146 | 6305 | −1101 | 358 | −808 | O |
| ATOM | 87 | N | ALA | A | 17 | 83.280 | 50.100 | 19.524 | 1.00 | 48.20 | | N |
| ANISOU | 87 | N | ALA | A | 17 | 3441 | 9202 | 5672 | −1587 | 564 | −924 | N |
| ATOM | 88 | CA | ALA | A | 17 | 84.197 | 49.930 | 20.648 | 1.00 | 48.23 | | C |
| ANISOU | 88 | CA | ALA | A | 17 | 3254 | 9517 | 5552 | −1634 | 449 | −1100 | C |
| ATOM | 89 | CB | ALA | A | 17 | 84.066 | 51.104 | 21.596 | 1.00 | 49.29 | | C |
| ANISOU | 89 | CB | ALA | A | 17 | 3474 | 9539 | 5716 | −1937 | 547 | −1209 | C |
| ATOM | 90 | C | ALA | A | 17 | 84.068 | 48.595 | 21.428 | 1.00 | 51.35 | | C |
| ANISOU | 90 | C | ALA | A | 17 | 3648 | 10017 | 5846 | −1310 | 236 | −1087 | C |
| ATOM | 91 | O | ALA | A | 17 | 85.104 | 48.035 | 21.816 | 1.00 | 51.89 | | O |
| ANISOU | 91 | O | ALA | A | 17 | 3497 | 10448 | 5771 | −1222 | 110 | −1206 | O |
| ATOM | 92 | N | TYR | A | 18 | 82.812 | 48.094 | 21.637 | 1.00 | 45.43 | | N |
| ANISOU | 92 | N | TYR | A | 18 | 3145 | 8960 | 5156 | −1134 | 204 | −947 | N |
| ATOM | 93 | CA | TYR | A | 18 | 82.442 | 46.850 | 22.346 | 1.00 | 43.82 | | C |
| ANISOU | 93 | CA | TYR | A | 18 | 3025 | 8761 | 4864 | −845 | 39 | −909 | C |
| ATOM | 94 | CB | TYR | A | 18 | 80.921 | 46.811 | 22.571 | 1.00 | 44.13 | | C |
| ANISOU | 94 | CB | TYR | A | 18 | 3346 | 8416 | 5006 | −803 | 79 | −780 | C |
| ATOM | 95 | CG | TYR | A | 18 | 80.115 | 46.541 | 21.314 | 1.00 | 44.88 | | C |
| ANISOU | 95 | CG | TYR | A | 18 | 3547 | 8305 | 5199 | −712 | 141 | −622 | C |
| ATOM | 96 | CD2 | TYR | A | 18 | 79.608 | 45.273 | 21.045 | 1.00 | 45.22 | | C |
| ANISOU | 96 | CD2 | TYR | A | 18 | 3684 | 8295 | 5201 | −454 | 46 | −526 | C |
| ATOM | 97 | CE2 | TYR | A | 18 | 78.888 | 45.016 | 19.879 | 1.00 | 45.73 | | C |
| ANISOU | 97 | CE2 | TYR | A | 18 | 3831 | 8202 | 5342 | −381 | 95 | −398 | C |
| ATOM | 98 | CZ | TYR | A | 18 | 78.652 | 46.042 | 18.978 | 1.00 | 50.32 | | C |
| ANISOU | 98 | CZ | TYR | A | 18 | 4407 | 8675 | 6038 | −537 | 236 | −353 | C |
| ATOM | 99 | OH | TYR | A | 18 | 77.912 | 45.816 | 17.849 | 1.00 | 49.15 | | O |
| ANISOU | 99 | OH | TYR | A | 18 | 4337 | 8385 | 5951 | −452 | 277 | −231 | O |
| ATOM | 100 | CE1 | TYR | A | 18 | 79.129 | 47.313 | 19.234 | 1.00 | 45.33 | | C |
| ANISOU | 100 | CE1 | TYR | A | 18 | 3712 | 8065 | 5445 | −782 | 344 | −435 | C |
| ATOM | 101 | CD1 | TYR | A | 18 | 79.851 | 47.558 | 20.398 | 1.00 | 46.51 | | C |
| ANISOU | 101 | CD1 | TYR | A | 18 | 3777 | 8372 | 5524 | −883 | 298 | −574 | C |
| ATOM | 102 | C | TYR | A | 18 | 82.895 | 45.536 | 21.657 | 1.00 | 47.31 | | C |
| ANISOU | 102 | C | TYR | A | 18 | 3388 | 9380 | 5206 | −545 | −77 | −856 | C |
| ATOM | 103 | O | TYR | A | 18 | 82.753 | 44.464 | 22.254 | 1.00 | 45.66 | | O |
| ANISOU | 103 | O | TYR | A | 18 | 3254 | 9206 | 4889 | −299 | −210 | −841 | O |
| ATOM | 104 | N | LEU | A | 19 | 83.367 | 45.618 | 20.387 | 1.00 | 45.15 | | N |
| ANISOU | 104 | N | LEU | A | 19 | 3000 | 9192 | 4965 | −557 | −14 | −820 | N |
| ATOM | 105 | CA | LEU | A | 19 | 83.850 | 44.489 | 19.573 | 1.00 | 45.44 | | C |
| ANISOU | 105 | CA | LEU | A | 19 | 2962 | 9395 | 4908 | −284 | −101 | −773 | C |
| ATOM | 106 | CB | LEU | A | 19 | 83.487 | 44.681 | 18.092 | 1.00 | 44.75 | | C |
| ANISOU | 106 | CB | LEU | A | 19 | 2914 | 9162 | 4925 | −305 | 9 | −654 | C |
| ATOM | 107 | CG | LEU | A | 19 | 82.020 | 44.653 | 17.686 | 1.00 | 48.60 | | C |
| ANISOU | 107 | CG | LEU | A | 19 | 3663 | 9262 | 5543 | −290 | 70 | −502 | C |
| ATOM | 108 | CD1 | LEU | A | 19 | 81.872 | 45.102 | 16.244 | 1.00 | 48.44 | | C |
| ANISOU | 108 | CD1 | LEU | A | 19 | 3635 | 9161 | 5611 | −358 | 193 | −419 | C |
| ATOM | 109 | CD2 | LEU | A | 19 | 81.417 | 43.257 | 17.839 | 1.00 | 49.92 | | C |
| ANISOU | 109 | CD2 | LEU | A | 19 | 3996 | 9328 | 5643 | −6 | −53 | −421 | C |
| ATOM | 110 | C | LEU | A | 19 | 85.373 | 44.307 | 19.678 | 1.00 | 53.13 | | C |
| ANISOU | 110 | C | LEU | A | 19 | 3632 | 10824 | 5730 | −246 | −173 | −920 | C |
| ATOM | 111 | O | LEU | A | 19 | 86.106 | 45.293 | 19.802 | 1.00 | 53.17 | | O |
| ANISOU | 111 | O | LEU | A | 19 | 3446 | 11009 | 5748 | −513 | −97 | −1044 | O |
| ATOM | 112 | N | ALA | A | 20 | 85.847 | 43.045 | 19.585 | 1.00 | 51.08 | | N |
| ANISOU | 112 | N | ALA | A | 20 | 3333 | 10759 | 5317 | 86 | −310 | −909 | N |
| ATOM | 113 | CA | ALA | A | 20 | 87.274 | 42.700 | 19.644 | 1.00 | 51.18 | | C |
| ANISOU | 113 | CA | ALA | A | 20 | 3046 | 11245 | 5157 | 201 | −396 | −1042 | C |
| ATOM | 114 | CB | ALA | A | 20 | 87.437 | 41.193 | 19.761 | 1.00 | 51.68 | | C |
| ANISOU | 114 | CB | ALA | A | 20 | 3189 | 11408 | 5041 | 634 | −554 | −997 | C |
| ATOM | 115 | C | ALA | A | 20 | 87.992 | 43.219 | 18.385 | 1.00 | 57.80 | | C |
| ANISOU | 115 | C | ALA | A | 20 | 3670 | 12250 | 6042 | 61 | −281 | −1057 | C |
| ATOM | 116 | O | ALA | A | 20 | 87.358 | 43.290 | 17.335 | 1.00 | 57.71 | | O |
| ANISOU | 116 | O | ALA | A | 20 | 3797 | 11979 | 6150 | 36 | −184 | −926 | O |
| ATOM | 117 | N | PRO | A | 21 | 89.287 | 43.604 | 18.455 | 1.00 | 55.12 | | N |
| ANISOU | 117 | N | PRO | A | 21 | 2992 | 12349 | 5604 | −41 | −283 | −1221 | N |
| ATOM | 118 | CA | PRO | A | 21 | 89.983 | 44.153 | 17.264 | 1.00 | 54.73 | | C |
| ANISOU | 118 | CA | PRO | A | 21 | 2736 | 12466 | 5594 | −209 | −150 | −1245 | C |
| ATOM | 119 | CB | PRO | A | 21 | 91.472 | 44.009 | 17.621 | 1.00 | 56.72 | | C |
| ANISOU | 119 | CB | PRO | A | 21 | 2596 | 13300 | 5655 | −167 | −232 | −1439 | C |
| ATOM | 120 | CG | PRO | A | 21 | 91.516 | 43.392 | 19.001 | 1.00 | 61.40 | | C |
| ANISOU | 120 | CG | PRO | A | 21 | 3215 | 14014 | 6102 | 60 | −420 | −1506 | C |
| ATOM | 121 | CD | PRO | A | 21 | 90.177 | 43.622 | 19.626 | 1.00 | 56.58 | | C |
| ANISOU | 121 | CD | PRO | A | 21 | 2953 | 12918 | 5628 | −31 | −398 | −1405 | C |
| ATOM | 122 | C | PRO | A | 21 | 89.683 | 43.511 | 15.900 | 1.00 | 56.98 | | C |
| ANISOU | 122 | C | PRO | A | 21 | 3126 | 12610 | 5912 | −7 | −113 | −1086 | C |
| ATOM | 123 | O | PRO | A | 21 | 89.400 | 44.236 | 14.941 | 1.00 | 56.31 | | O |
| ANISOU | 123 | O | PRO | A | 21 | 3086 | 12351 | 5959 | −216 | 48 | −1021 | O |
| ATOM | 124 | N | GLU | A | 22 | 89.760 | 42.168 | 15.813 | 1.00 | 52.66 | | N |
| ANISOU | 124 | N | GLU | A | 22 | 2640 | 12138 | 5232 | 401 | −257 | −1029 | N |
| ATOM | 125 | CA | GLU | A | 22 | 89.522 | 41.367 | 14.597 | 1.00 | 51.71 | | C |
| ANISOU | 125 | CA | GLU | A | 22 | 2638 | 11902 | 5108 | 639 | −248 | −890 | C |
| ATOM | 126 | CB | GLU | A | 22 | 90.061 | 39.919 | 14.725 | 1.00 | 53.49 | | C |
| ANISOU | 126 | CB | GLU | A | 22 | 2866 | 12340 | 5118 | 1095 | −415 | −889 | C |
| ATOM | 127 | CG | GLU | A | 22 | 89.658 | 39.161 | 15.998 | 1.00 | 75.35 | | C |
| ANISOU | 127 | CG | GLU | A | 22 | 5824 | 15019 | 7786 | 1303 | −562 | −893 | C |
| ATOM | 128 | CD | GLU | A | 22 | 90.275 | 39.520 | 17.354 | 1.00 | 105.43 | | C |
| ANISOU | 128 | CD | GLU | A | 22 | 9444 | 19120 | 11494 | 1241 | −645 | −1056 | C |
| ATOM | 129 | OE1 | GLU | A | 22 | 91.447 | 39.966 | 17.401 | 1.00 | 106.33 | | O |
| ANISOU | 129 | OE1 | GLU | A | 22 | 9199 | 19678 | 11523 | 1162 | −645 | −1206 | O |
| ATOM | 130 | OE2 | GLU | A | 22 | 89.583 | 39.326 | 18.381 | 1.00 | 90.91 | | O |
| ANISOU | 130 | OE2 | GLU | A | 22 | 7814 | 17078 | 9650 | 1273 | −710 | −1039 | O |
| ATOM | 131 | C | GLU | A | 22 | 88.062 | 41.393 | 14.182 | 1.00 | 53.74 | | C |
| ANISOU | 131 | C | GLU | A | 22 | 3239 | 11650 | 5529 | 592 | −185 | −722 | C |
| ATOM | 132 | O | GLU | A | 22 | 87.759 | 41.252 | 13.003 | 1.00 | 55.75 | | O |
| ANISOU | 132 | O | GLU | A | 22 | 3571 | 11769 | 5842 | 626 | −114 | −618 | O |
| ATOM | 133 | N | GLU | A | 23 | 87.160 | 41.590 | 15.140 | 1.00 | 47.40 | | N |
| ANISOU | 133 | N | GLU | A | 23 | 2630 | 10588 | 4794 | 513 | −211 | −703 | N |
| ATOM | 134 | CA | GLU | A | 23 | 85.734 | 41.721 | 14.888 | 1.00 | 45.88 | | C |
| ANISOU | 134 | CA | GLU | A | 23 | 2730 | 9949 | 4753 | 445 | −150 | −564 | C |
| ATOM | 135 | CB | GLU | A | 23 | 84.923 | 41.594 | 16.191 | 1.00 | 47.54 | | C |
| ANISOU | 135 | CB | GLU | A | 23 | 3129 | 9960 | 4975 | 444 | −218 | −565 | C |
| ATOM | 136 | CG | GLU | A | 23 | 84.760 | 40.180 | 16.721 | 1.00 | 57.75 | | C |
| ANISOU | 136 | CG | GLU | A | 23 | 4577 | 11248 | 6117 | 780 | −368 | −537 | C |
| ATOM | 137 | CD | GLU | A | 23 | 83.697 | 40.070 | 17.795 | 1.00 | 85.09 | | C |
| ANISOU | 137 | CD | GLU | A | 23 | 8279 | 14436 | 9614 | 755 | −399 | −506 | C |
| ATOM | 138 | OE1 | GLU | A | 23 | 83.954 | 40.524 | 18.935 | 1.00 | 94.51 | | O |
| ANISOU | 138 | OE1 | GLU | A | 23 | 9407 | 15725 | 10777 | 660 | −429 | −603 | O |
| ATOM | 139 | OE2 | GLU | A | 23 | 82.603 | 39.537 | 17.495 | 1.00 | 77.32 | | O |
| ANISOU | 139 | OE2 | GLU | A | 23 | 7543 | 13153 | 8682 | 821 | −388 | −392 | O |
| ATOM | 140 | C | GLU | A | 23 | 85.514 | 43.094 | 14.263 | 1.00 | 46.18 | | C |
| ANISOU | 140 | C | GLU | A | 23 | 2731 | 9863 | 4953 | 109 | 29 | −550 | C |
| ATOM | 141 | O | GLU | A | 23 | 84.715 | 43.200 | 13.339 | 1.00 | 47.56 | | O |
| ANISOU | 141 | O | GLU | A | 23 | 3059 | 9786 | 5227 | 99 | 106 | −427 | O |
| ATOM | 142 | N | ARG | A | 24 | 86.242 | 44.135 | 14.727 | 1.00 | 39.08 | | N |
| ANISOU | 142 | N | ARG | A | 24 | 1639 | 9145 | 4063 | −160 | 100 | −680 | N |
| ATOM | 143 | CA | ARG | A | 24 | 86.126 | 45.472 | 14.154 | 1.00 | 38.60 | | C |
| ANISOU | 143 | CA | ARG | A | 24 | 1579 | 8956 | 4134 | −487 | 292 | −674 | C |
| ATOM | 144 | CB | ARG | A | 24 | 86.774 | 46.553 | 15.027 | 1.00 | 38.86 | | C |
| ANISOU | 144 | CB | ARG | A | 24 | 1460 | 9141 | 4164 | −804 | 357 | −836 | C |
| ATOM | 145 | CG | ARG | A | 24 | 85.937 | 46.994 | 16.250 | 1.00 | 53.23 | | C |
| ANISOU | 145 | CG | ARG | A | 24 | 3458 | 10723 | 6043 | −911 | 340 | −850 | C |
| ATOM | 146 | CD | ARG | A | 24 | 86.428 | 48.288 | 16.908 | 1.00 | 56.02 | | C |
| ANISOU | 146 | CD | ARG | A | 24 | 3718 | 11150 | 6417 | −1285 | 452 | −1000 | C |
| ATOM | 147 | NE | ARG | A | 24 | 87.870 | 48.263 | 17.131 | 1.00 | 64.69 | | N |
| ANISOU | 147 | NE | ARG | A | 24 | 4480 | 12718 | 7381 | −1358 | 409 | −1180 | N |
| ATOM | 148 | CZ | ARG | A | 24 | 88.457 | 47.780 | 18.219 | 1.00 | 85.83 | | C |
| ANISOU | 148 | CZ | ARG | A | 24 | 7001 | 15682 | 9930 | −1269 | 254 | −1312 | C |
| ATOM | 149 | NH1 | ARG | A | 24 | 87.727 | 47.327 | 19.232 | 1.00 | 71.39 | | N |
| ANISOU | 149 | NH1 | ARG | A | 24 | 5347 | 13687 | 8092 | −1128 | 140 | −1282 | N |
| ATOM | 150 | NH2 | ARG | A | 24 | 89.781 | 47.774 | 18.318 | 1.00 | 78.28 | | N |
| ANISOU | 150 | NH2 | ARG | A | 24 | 5708 | 15196 | 8840 | −1324 | 216 | −1480 | N |
| ATOM | 151 | C | ARG | A | 24 | 86.713 | 45.466 | 12.740 | 1.00 | 44.59 | | C |
| ANISOU | 151 | C | ARG | A | 24 | 2225 | 9835 | 4882 | −473 | 382 | −634 | C |
| ATOM | 152 | O | ARG | A | 24 | 86.040 | 45.917 | 11.815 | 1.00 | 45.38 | | O |
| ANISOU | 152 | O | ARG | A | 24 | 2478 | 9676 | 5086 | −548 | 505 | −521 | O |
| ATOM | 153 | N | ALA | A | 25 | 87.911 | 44.868 | 12.547 | 1.00 | 40.84 | | N |
| ANISOU | 153 | N | ALA | A | 25 | 1495 | 9755 | 4267 | −339 | 316 | −721 | N |
| ATOM | 154 | CA | ALA | A | 25 | 88.542 | 44.744 | 11.226 | 1.00 | 40.52 | | C |
| ANISOU | 154 | CA | ALA | A | 25 | 1334 | 9866 | 4198 | −297 | 396 | −689 | C |
| ATOM | 155 | CB | ALA | A | 25 | 89.809 | 43.914 | 11.318 | 1.00 | 41.36 | | C |
| ANISOU | 155 | CB | ALA | A | 25 | 1149 | 10443 | 4122 | −92 | 286 | −801 | C |
| ATOM | 156 | C | ALA | A | 25 | 87.588 | 44.134 | 10.181 | 1.00 | 43.76 | | C |
| ANISOU | 156 | C | ALA | A | 25 | 1990 | 9975 | 4662 | −88 | 395 | −506 | C |
| ATOM | 157 | O | ALA | A | 25 | 87.622 | 44.561 | 9.018 | 1.00 | 43.19 | | O |
| ANISOU | 157 | O | ALA | A | 25 | 1934 | 9837 | 4639 | −169 | 530 | −439 | O |
| ATOM | 158 | N | LYS | A | 26 | 86.722 | 43.156 | 10.609 | 1.00 | 38.75 | | N |
| ANISOU | 158 | N | LYS | A | 26 | 1560 | 9154 | 4011 | 164 | 253 | −430 | N |
| ATOM | 159 | CA | LYS | A | 26 | 85.716 | 42.531 | 9.738 | 1.00 | 37.10 | | C |
| ANISOU | 159 | CA | LYS | A | 26 | 1592 | 8660 | 3845 | 339 | 240 | −275 | C |
| ATOM | 160 | CB | LYS | A | 26 | 84.974 | 41.365 | 10.421 | 1.00 | 38.88 | | C |
| ANISOU | 160 | CB | LYS | A | 26 | 2015 | 8739 | 4017 | 584 | 83 | −234 | C |
| ATOM | 161 | CG | LYS | A | 26 | 84.156 | 40.552 | 9.398 | 1.00 | 46.17 | | C |
| ANISOU | 161 | CG | LYS | A | 26 | 3146 | 9451 | 4945 | 774 | 63 | −103 | C |
| ATOM | 162 | CD | LYS | A | 26 | 83.244 | 39.501 | 10.013 | 1.00 | 49.44 | | C |
| ANISOU | 162 | CD | LYS | A | 26 | 3799 | 9664 | 5321 | 948 | −56 | −59 | C |
| ATOM | 163 | CE | LYS | A | 26 | 83.136 | 38.272 | 9.148 | 1.00 | 47.79 | | C |
| ANISOU | 163 | CE | LYS | A | 26 | 3729 | 9408 | 5021 | 1205 | −113 | 10 | C |
| ATOM | 164 | NZ | LYS | A | 26 | 84.231 | 37.294 | 9.409 | 1.00 | 55.18 | | N |
| ANISOU | 164 | NZ | LYS | A | 26 | 4596 | 10603 | 5768 | 1475 | −213 | −50 | N |
| ATOM | 165 | C | LYS | A | 26 | 84.730 | 43.591 | 9.305 | 1.00 | 37.34 | | C |
| ANISOU | 165 | C | LYS | A | 26 | 1782 | 8370 | 4035 | 114 | 383 | −189 | C |
| ATOM | 166 | O | LYS | A | 26 | 84.464 | 43.702 | 8.120 | 1.00 | 36.10 | | O |
| ANISOU | 166 | O | LYS | A | 26 | 1691 | 8112 | 3913 | 134 | 463 | −97 | O |
| ATOM | 167 | N | VAL | A | 27 | 84.236 | 44.394 | 10.267 | 1.00 | 32.92 | | N |
| ANISOU | 167 | N | VAL | A | 27 | 1285 | 7666 | 3557 | −88 | 418 | −224 | N |
| ATOM | 168 | CA | VAL | A | 27 | 83.297 | 45.486 | 10.033 | 1.00 | 32.18 | | C |
| ANISOU | 168 | CA | VAL | A | 27 | 1358 | 7270 | 3599 | −284 | 557 | −150 | C |
| ATOM | 169 | CB | VAL | A | 27 | 82.708 | 46.059 | 11.345 | 1.00 | 34.70 | | C |
| ANISOU | 169 | CB | VAL | A | 27 | 1765 | 7441 | 3978 | −432 | 553 | −196 | C |
| ATOM | 170 | CG1 | VAL | A | 27 | 81.814 | 47.257 | 11.071 | 1.00 | 34.18 | | C |
| ANISOU | 170 | CG1 | VAL | A | 27 | 1879 | 7075 | 4033 | −609 | 710 | −122 | C |
| ATOM | 171 | CG2 | VAL | A | 27 | 81.937 | 44.992 | 12.107 | 1.00 | 33.62 | | C |
| ANISOU | 171 | CG2 | VAL | A | 27 | 1747 | 7214 | 3813 | −225 | 392 | −169 | C |
| ATOM | 172 | C | VAL | A | 27 | 83.913 | 46.563 | 9.112 | 1.00 | 38.11 | | C |
| ANISOU | 172 | C | VAL | A | 27 | 2032 | 8067 | 4379 | −496 | 746 | −155 | C |
| ATOM | 173 | O | VAL | A | 27 | 83.215 | 46.978 | 8.182 | 1.00 | 40.19 | | O |
| ANISOU | 173 | O | VAL | A | 27 | 2452 | 8112 | 4707 | −499 | 845 | −40 | O |
| ATOM | 174 | N | ARG | A | 28 | 85.204 | 46.968 | 9.299 | 1.00 | 32.77 | | N |
| ANISOU | 174 | N | ARG | A | 28 | 1120 | 7687 | 3643 | −662 | 799 | −287 | N |
| ATOM | 175 | CA | ARG | A | 28 | 85.813 | 47.951 | 8.388 | 1.00 | 32.63 | | C |
| ANISOU | 175 | CA | ARG | A | 28 | 1044 | 7713 | 3642 | −887 | 999 | −297 | C |
| ATOM | 176 | CB | ARG | A | 28 | 87.173 | 48.441 | 8.894 | 1.00 | 30.06 | | C |
| ANISOU | 176 | CB | ARG | A | 28 | 800 | 7531 | 3089 | −1054 | 880 | −500 | C |
| ATOM | 177 | CG | ARG | A | 28 | 88.033 | 49.241 | 7.882 | 1.00 | 32.53 | | C |
| ANISOU | 177 | CG | ARG | A | 28 | 960 | 7987 | 3413 | −1283 | 1094 | −532 | C |
| ATOM | 178 | CD | ARG | A | 28 | 87.453 | 50.568 | 7.408 | 1.00 | 33.08 | | C |
| ANISOU | 178 | CD | ARG | A | 28 | 1208 | 7748 | 3612 | −1547 | 1337 | −467 | C |
| ATOM | 179 | NE | ARG | A | 28 | 88.494 | 51.475 | 6.917 | 1.00 | 46.88 | | N |
| ANISOU | 179 | NE | ARG | A | 28 | 2588 | 9805 | 5419 | −1938 | 1703 | −531 | N |
| ATOM | 180 | CZ | ARG | A | 28 | 88.267 | 52.697 | 6.436 | 1.00 | 71.51 | | C |
| ANISOU | 180 | CZ | ARG | A | 28 | 5917 | 12666 | 8586 | −2209 | 1945 | −493 | C |
| ATOM | 181 | NH1 | ARG | A | 28 | 87.031 | 53.179 | 6.381 | 1.00 | 58.62 | | N |
| ANISOU | 181 | NH1 | ARG | A | 28 | 4621 | 10612 | 7038 | −2169 | 1995 | −362 | N |
| ATOM | 182 | NH2 | ARG | A | 28 | 89.275 | 53.449 | 6.008 | 1.00 | 64.97 | | N |
| ANISOU | 182 | NH2 | ARG | A | 28 | 4971 | 12005 | 7709 | −2517 | 2145 | −589 | N |
| ATOM | 183 | C | ARG | A | 28 | 85.887 | 47.436 | 6.933 | 1.00 | 40.36 | | C |
| ANISOU | 183 | C | ARG | A | 28 | 2035 | 8712 | 4589 | −704 | 1025 | −190 | C |
| ATOM | 184 | O | ARG | A | 28 | 85.563 | 48.188 | 6.001 | 1.00 | 41.04 | | O |
| ANISOU | 184 | O | ARG | A | 28 | 2261 | 8603 | 4728 | −799 | 1183 | −100 | O |
| ATOM | 185 | N | GLU | A | 29 | 86.289 | 46.147 | 6.758 | 1.00 | 37.58 | | N |
| ANISOU | 185 | N | GLU | A | 29 | 1561 | 8580 | 4138 | −424 | 870 | −198 | N |
| ATOM | 186 | CA | GLU | A | 29 | 86.377 | 45.413 | 5.481 | 1.00 | 36.54 | | C |
| ANISOU | 186 | CA | GLU | A | 29 | 1443 | 8488 | 3953 | −200 | 860 | −107 | C |
| ATOM | 187 | CB | GLU | A | 29 | 86.990 | 44.018 | 5.722 | 1.00 | 37.32 | | C |
| ANISOU | 187 | CB | GLU | A | 29 | 1410 | 8852 | 3920 | 101 | 676 | −156 | C |
| ATOM | 188 | CG | GLU | A | 29 | 87.001 | 43.030 | 4.566 | 1.00 | 42.42 | | C |
| ANISOU | 188 | CG | GLU | A | 29 | 2102 | 9526 | 4491 | 380 | 633 | −69 | C |
| ATOM | 189 | CD | GLU | A | 29 | 87.484 | 41.633 | 4.929 | 1.00 | 69.59 | | C |
| ANISOU | 189 | CD | GLU | A | 29 | 5475 | 13182 | 7786 | 700 | 455 | −113 | C |
| ATOM | 190 | OE1 | GLU | A | 29 | 87.124 | 41.136 | 6.023 | 1.00 | 87.67 | | O |
| ANISOU | 190 | OE1 | GLU | A | 29 | 7833 | 15423 | 10056 | 787 | 325 | −144 | O |
| ATOM | 191 | OE2 | GLU | A | 29 | 88.217 | 41.026 | 4.113 | 1.00 | 55.99 | | O |
| ANISOU | 191 | OE2 | GLU | A | 29 | 3650 | 11667 | 5956 | 879 | 452 | −113 | O |
| ATOM | 192 | C | GLU | A | 29 | 84.979 | 45.319 | 4.855 | 1.00 | 41.93 | | C |
| ANISOU | 192 | C | GLU | A | 29 | 2414 | 8803 | 4715 | −89 | 860 | 52 | C |
| ATOM | 193 | O | GLU | A | 29 | 84.867 | 45.463 | 3.641 | 1.00 | 43.12 | | O |
| ANISOU | 193 | O | GLU | A | 29 | 2632 | 8885 | 4867 | −55 | 952 | 140 | O |
| ATOM | 194 | N | ALA | A | 30 | 83.918 | 45.122 | 5.682 | 1.00 | 37.72 | | N |
| ANISOU | 194 | N | ALA | A | 30 | 2042 | 8052 | 4240 | −43 | 764 | 82 | N |
| ATOM | 195 | CA | ALA | A | 30 | 82.526 | 45.081 | 5.224 | 1.00 | 37.39 | | C |
| ANISOU | 195 | CA | ALA | A | 30 | 2243 | 7697 | 4268 | 42 | 760 | 212 | C |
| ATOM | 196 | CB | ALA | A | 30 | 81.617 | 44.585 | 6.325 | 1.00 | 38.05 | | C |
| ANISOU | 196 | CB | ALA | A | 30 | 2435 | 7640 | 4382 | 106 | 630 | 207 | C |
| ATOM | 197 | C | ALA | A | 30 | 82.079 | 46.450 | 4.751 | 1.00 | 42.02 | | C |
| ANISOU | 197 | C | ALA | A | 30 | 2944 | 8083 | 4940 | −158 | 947 | 271 | C |
| ATOM | 198 | O | ALA | A | 30 | 81.412 | 46.539 | 3.720 | 1.00 | 40.18 | | O |
| ANISOU | 198 | O | ALA | A | 30 | 2840 | 7707 | 4717 | −74 | 999 | 381 | O |
| ATOM | 199 | N | TYR | A | 31 | 82.465 | 47.520 | 5.497 | 1.00 | 40.86 | | N |
| ANISOU | 199 | N | TYR | A | 31 | 2761 | 7928 | 4834 | −417 | 1053 | 194 | N |
| ATOM | 200 | CA | TYR | A | 31 | 82.163 | 48.909 | 5.142 | 1.00 | 41.17 | | C |
| ANISOU | 200 | CA | TYR | A | 31 | 2944 | 7764 | 4935 | −624 | 1258 | 238 | C |
| ATOM | 201 | CB | TYR | A | 31 | 82.540 | 49.898 | 6.259 | 1.00 | 42.67 | | C |
| ANISOU | 201 | CB | TYR | A | 31 | 3107 | 7948 | 5158 | −910 | 1344 | 125 | C |
| ATOM | 202 | CG | TYR | A | 31 | 82.705 | 51.317 | 5.747 | 1.00 | 45.02 | | C |
| ANISOU | 202 | CG | TYR | A | 31 | 3533 | 8094 | 5478 | −1161 | 1593 | 143 | C |
| ATOM | 203 | CD1 | TYR | A | 31 | 81.599 | 52.119 | 5.491 | 1.00 | 47.29 | | C |
| ANISOU | 203 | CD1 | TYR | A | 31 | 4100 | 8047 | 5818 | −1149 | 1698 | 257 | C |
| ATOM | 204 | CE1 | TYR | A | 31 | 81.740 | 53.412 | 4.990 | 1.00 | 48.47 | | C |
| ANISOU | 204 | CE1 | TYR | A | 31 | 4422 | 8027 | 5967 | −1353 | 1941 | 285 | C |
| ATOM | 205 | CZ | TYR | A | 31 | 83.002 | 53.920 | 4.740 | 1.00 | 56.44 | | C |
| ANISOU | 205 | CZ | TYR | A | 31 | 5313 | 9201 | 6930 | −1615 | 2092 | 189 | C |
| ATOM | 206 | OH | TYR | A | 31 | 83.151 | 55.200 | 4.247 | 1.00 | 59.73 | | O |
| ANISOU | 206 | OH | TYR | A | 31 | 5938 | 9424 | 7334 | −1842 | 2355 | 213 | O |
| ATOM | 207 | CE2 | TYR | A | 31 | 84.116 | 53.137 | 4.989 | 1.00 | 47.46 | | C |
| ANISOU | 207 | CE2 | TYR | A | 31 | 3855 | 8432 | 5746 | −1645 | 1986 | 64 | C |
| ATOM | 208 | CD2 | TYR | A | 31 | 83.962 | 51.840 | 5.478 | 1.00 | 45.99 | | C |
| ANISOU | 208 | CD2 | TYR | A | 31 | 3509 | 8412 | 5552 | −1396 | 1734 | 47 | C |
| ATOM | 209 | C | TYR | A | 31 | 82.876 | 49.296 | 3.858 | 1.00 | 44.18 | | C |
| ANISOU | 209 | C | TYR | A | 31 | 3294 | 8222 | 5272 | −677 | 1411 | 273 | C |
| ATOM | 210 | O | TYR | A | 31 | 82.277 | 49.957 | 3.014 | 1.00 | 44.07 | | O |
| ANISOU | 210 | O | TYR | A | 31 | 3471 | 7993 | 5279 | −677 | 1538 | 382 | O |
| ATOM | 211 | N | ARG | A | 32 | 84.160 | 48.929 | 3.728 | 1.00 | 40.17 | | N |
| ANISOU | 211 | N | ARG | A | 32 | 2545 | 8027 | 4689 | −717 | 1406 | 178 | N |
| ATOM | 212 | CA | ARG | A | 32 | 84.941 | 49.249 | 2.533 | 1.00 | 39.68 | | C |
| ANISOU | 212 | CA | ARG | A | 32 | 2428 | 8075 | 4575 | −777 | 1558 | 199 | C |
| ATOM | 213 | CB | ARG | A | 32 | 86.432 | 49.078 | 2.777 | 1.00 | 36.84 | | C |
| ANISOU | 213 | CB | ARG | A | 32 | 1757 | 8104 | 4136 | −900 | 1571 | 47 | C |
| ATOM | 214 | CG | ARG | A | 32 | 87.032 | 50.421 | 3.069 | 1.00 | 43.13 | | C |
| ANISOU | 214 | CG | ARG | A | 32 | 2533 | 8904 | 4952 | −1290 | 1788 | −44 | C |
| ATOM | 215 | CD | ARG | A | 32 | 88.510 | 50.347 | 3.183 | 1.00 | 55.22 | | C |
| ANISOU | 215 | CD | ARG | A | 32 | 3730 | 10855 | 6396 | −1442 | 1819 | −207 | C |
| ATOM | 216 | NE | ARG | A | 32 | 89.186 | 50.421 | 1.886 | 1.00 | 63.69 | | N |
| ANISOU | 216 | NE | ARG | A | 32 | 4734 | 12058 | 7406 | −1471 | 1969 | −177 | N |
| ATOM | 217 | CZ | ARG | A | 32 | 89.630 | 51.547 | 1.342 | 1.00 | 69.57 | | C |
| ANISOU | 217 | CZ | ARG | A | 32 | 5537 | 12749 | 8149 | −1789 | 2230 | −196 | C |
| ATOM | 218 | NH1 | ARG | A | 32 | 89.422 | 52.714 | 1.944 | 1.00 | 53.03 | | N |
| ANISOU | 218 | NH1 | ARG | A | 32 | 3598 | 10441 | 6111 | −2100 | 2373 | −237 | N |
| ATOM | 219 | NH2 | ARG | A | 32 | 90.262 | 51.520 | 0.182 | 1.00 | 55.66 | | N |
| ANISOU | 219 | NH2 | ARG | A | 32 | 3704 | 11125 | 6320 | −1800 | 2363 | −170 | N |
| ATOM | 220 | C | ARG | A | 32 | 84.490 | 48.495 | 1.301 | 1.00 | 44.76 | | C |
| ANISOU | 220 | C | ARG | A | 32 | 3159 | 8666 | 5183 | −497 | 1506 | 328 | C |
| ATOM | 221 | O | ARG | A | 32 | 84.808 | 48.905 | 0.187 | 1.00 | 47.27 | | O |
| ANISOU | 221 | O | ARG | A | 32 | 3519 | 8974 | 5467 | −529 | 1653 | 386 | O |
| ATOM | 222 | N | PHE | A | 33 | 83.747 | 47.402 | 1.501 | 1.00 | 39.88 | | N |
| ANISOU | 222 | N | PHE | A | 33 | 2583 | 8008 | 4563 | −238 | 1305 | 368 | N |
| ATOM | 223 | CA | PHE | A | 33 | 83.182 | 46.565 | 0.452 | 1.00 | 39.26 | | C |
| ANISOU | 223 | CA | PHE | A | 33 | 2602 | 7869 | 4444 | 26 | 1230 | 476 | C |
| ATOM | 224 | CB | PHE | A | 33 | 82.892 | 45.171 | 1.010 | 1.00 | 40.53 | | C |
| ANISOU | 224 | CB | PHE | A | 33 | 2730 | 8100 | 4569 | 257 | 1003 | 451 | C |
| ATOM | 225 | CG | PHE | A | 33 | 82.371 | 44.181 | 0.013 | 1.00 | 41.11 | | C |
| ANISOU | 225 | CG | PHE | A | 33 | 2903 | 8131 | 4586 | 514 | 914 | 536 | C |
| ATOM | 226 | CD2 | PHE | A | 33 | 83.192 | 43.191 | −0.491 | 1.00 | 43.13 | | C |
| ANISOU | 226 | CD2 | PHE | A | 33 | 3050 | 8603 | 4733 | 688 | 848 | 508 | C |
| ATOM | 227 | CE2 | PHE | A | 33 | 82.714 | 42.274 | −1.420 | 1.00 | 46.05 | | C |
| ANISOU | 227 | CE2 | PHE | A | 33 | 3535 | 8920 | 5041 | 915 | 770 | 578 | C |
| ATOM | 228 | CZ | PHE | A | 33 | 81.400 | 42.319 | −1.809 | 1.00 | 44.09 | | C |
| ANISOU | 228 | CZ | PHE | A | 33 | 3483 | 8431 | 4838 | 960 | 743 | 665 | C |
| ATOM | 229 | CE1 | PHE | A | 33 | 80.560 | 43.281 | −1.295 | 1.00 | 44.76 | | C |
| ANISOU | 229 | CE1 | PHE | A | 33 | 3653 | 8327 | 5028 | 807 | 798 | 693 | C |
| ATOM | 230 | CD1 | PHE | A | 33 | 81.047 | 44.215 | −0.395 | 1.00 | 43.62 | | C |
| ANISOU | 230 | CD1 | PHE | A | 33 | 3421 | 8207 | 4946 | 591 | 888 | 634 | C |
| ATOM | 231 | C | PHE | A | 33 | 81.897 | 47.233 | −0.014 | 1.00 | 44.92 | | C |
| ANISOU | 231 | C | PHE | A | 33 | 3571 | 8274 | 5224 | 40 | 1295 | 599 | C |
| ATOM | 232 | O | PHE | A | 33 | 81.745 | 47.494 | −1.205 | 1.00 | 44.57 | | O |
| ANISOU | 232 | O | PHE | A | 33 | 3627 | 8161 | 5148 | 100 | 1389 | 691 | O |
| ATOM | 233 | N | ALA | A | 34 | 80.970 | 47.508 | 0.937 | 1.00 | 43.24 | | N |
| ANISOU | 233 | N | ALA | A | 34 | 3459 | 7884 | 5086 | −4 | 1248 | 598 | N |
| ATOM | 234 | CA | ALA | A | 34 | 79.705 | 48.200 | 0.701 | 1.00 | 42.92 | | C |
| ANISOU | 234 | CA | ALA | A | 34 | 3641 | 7571 | 5095 | 24 | 1305 | 702 | C |
| ATOM | 235 | CB | ALA | A | 34 | 79.015 | 48.482 | 2.020 | 1.00 | 43.34 | | C |
| ANISOU | 235 | CB | ALA | A | 34 | 3745 | 7498 | 5223 | −47 | 1252 | 665 | C |
| ATOM | 236 | C | ALA | A | 34 | 80.014 | 49.507 | −0.028 | 1.00 | 47.61 | | C |
| ANISOU | 236 | C | ALA | A | 34 | 4347 | 8061 | 5683 | −128 | 1542 | 752 | C |
| ATOM | 237 | O | ALA | A | 34 | 79.419 | 49.737 | −1.082 | 1.00 | 48.16 | | O |
| ANISOU | 237 | O | ALA | A | 34 | 4573 | 8003 | 5724 | −4 | 1599 | 865 | O |
| ATOM | 238 | N | GLU | A | 35 | 81.049 | 50.280 | 0.454 | 1.00 | 43.20 | | N |
| ANISOU | 238 | N | GLU | A | 35 | 3702 | 7584 | 5129 | −393 | 1679 | 660 | N |
| ATOM | 239 | CA | GLU | A | 35 | 81.537 | 51.546 | −0.117 | 1.00 | 42.28 | | C |
| ANISOU | 239 | CA | GLU | A | 35 | 3700 | 7371 | 4994 | −601 | 1935 | 683 | C |
| ATOM | 240 | CB | GLU | A | 35 | 82.839 | 52.023 | 0.554 | 1.00 | 43.54 | | C |
| ANISOU | 240 | CB | GLU | A | 35 | 3683 | 7713 | 5148 | −913 | 2038 | 534 | C |
| ATOM | 241 | CG | GLU | A | 35 | 83.034 | 53.531 | 0.479 | 1.00 | 55.77 | | C |
| ANISOU | 241 | CG | GLU | A | 35 | 5425 | 9061 | 6704 | −1202 | 2304 | 531 | C |
| ATOM | 242 | CD | GLU | A | 35 | 84.444 | 54.077 | 0.647 | 1.00 | 78.39 | | C |
| ANISOU | 242 | CD | GLU | A | 35 | 8128 | 12125 | 9532 | −1545 | 2468 | 392 | C |
| ATOM | 243 | OE1 | GLU | A | 35 | 84.905 | 54.182 | 1.809 | 1.00 | 65.36 | | O |
| ANISOU | 243 | OE1 | GLU | A | 35 | 6325 | 10603 | 7907 | −1737 | 2425 | 247 | O |
| ATOM | 244 | OE2 | GLU | A | 35 | 85.072 | 54.437 | −0.379 | 1.00 | 67.54 | | O |
| ANISOU | 244 | OE2 | GLU | A | 35 | 6780 | 10785 | 8096 | −1632 | 2646 | 422 | O |
| ATOM | 245 | C | GLU | A | 35 | 81.738 | 51.419 | −1.602 | 1.00 | 46.50 | | C |
| ANISOU | 245 | C | GLU | A | 35 | 4286 | 7927 | 5454 | −479 | 2015 | 778 | C |
| ATOM | 246 | O | GLU | A | 35 | 81.306 | 52.303 | −2.344 | 1.00 | 46.33 | | O |
| ANISOU | 246 | O | GLU | A | 35 | 4498 | 7694 | 5410 | −486 | 2180 | 880 | O |
| ATOM | 247 | N | GLU | A | 36 | 82.349 | 50.295 | −2.043 | 1.00 | 43.79 | | N |
| ANISOU | 247 | N | GLU | A | 36 | 3747 | 7832 | 5058 | −340 | 1895 | 751 | N |
| ATOM | 248 | CA | GLU | A | 36 | 82.611 | 50.006 | −3.460 | 1.00 | 43.29 | | C |
| ANISOU | 248 | CA | GLU | A | 36 | 3710 | 7826 | 4914 | −202 | 1950 | 832 | C |
| ATOM | 249 | CB | GLU | A | 36 | 83.790 | 49.042 | −3.640 | 1.00 | 44.40 | | C |
| ANISOU | 249 | CB | GLU | A | 36 | 3581 | 8298 | 4989 | −158 | 1876 | 745 | C |
| ATOM | 250 | CG | GLU | A | 36 | 85.092 | 49.783 | −3.834 | 1.00 | 54.21 | | C |
| ANISOU | 250 | CG | GLU | A | 36 | 4694 | 9708 | 6195 | −427 | 2083 | 667 | C |
| ATOM | 251 | CD | GLU | A | 36 | 85.458 | 50.169 | −5.255 | 1.00 | 83.74 | | C |
| ANISOU | 251 | CD | GLU | A | 36 | 8524 | 13437 | 9858 | −428 | 2272 | 750 | C |
| ATOM | 252 | OE1 | GLU | A | 36 | 86.338 | 49.496 | −5.837 | 1.00 | 81.82 | | O |
| ANISOU | 252 | OE1 | GLU | A | 36 | 8093 | 13457 | 9537 | −354 | 2258 | 714 | O |
| ATOM | 253 | OE2 | GLU | A | 36 | 84.882 | 51.145 | −5.787 | 1.00 | 87.26 | | O |
| ANISOU | 253 | OE2 | GLU | A | 36 | 9235 | 13613 | 10306 | −488 | 2439 | 851 | O |
| ATOM | 254 | C | GLU | A | 36 | 81.379 | 49.516 | −4.195 | 1.00 | 46.42 | | C |
| ANISOU | 254 | C | GLU | A | 36 | 4273 | 8071 | 5292 | 87 | 1838 | 957 | C |
| ATOM | 255 | O | GLU | A | 36 | 81.031 | 50.095 | −5.225 | 1.00 | 46.17 | | O |
| ANISOU | 255 | O | GLU | A | 36 | 4423 | 7905 | 5213 | 150 | 1962 | 1066 | O |
| ATOM | 256 | N | ALA | A | 37 | 80.709 | 48.467 | −3.674 | 1.00 | 42.23 | | N |
| ANISOU | 256 | N | ALA | A | 37 | 3688 | 7571 | 4787 | 259 | 1611 | 937 | N |
| ATOM | 257 | CA | ALA | A | 37 | 79.506 | 47.931 | −4.299 | 1.00 | 41.80 | | C |
| ANISOU | 257 | CA | ALA | A | 37 | 3764 | 7407 | 4711 | 504 | 1493 | 1030 | C |
| ATOM | 258 | CB | ALA | A | 37 | 78.899 | 46.845 | −3.436 | 1.00 | 42.15 | | C |
| ANISOU | 258 | CB | ALA | A | 37 | 3739 | 7488 | 4788 | 609 | 1268 | 976 | C |
| ATOM | 259 | C | ALA | A | 37 | 78.488 | 49.047 | −4.592 | 1.00 | 47.81 | | C |
| ANISOU | 259 | C | ALA | A | 37 | 4756 | 7920 | 5490 | 509 | 1606 | 1130 | C |
| ATOM | 260 | O | ALA | A | 37 | 78.107 | 49.228 | −5.748 | 1.00 | 48.10 | | O |
| ANISOU | 260 | O | ALA | A | 37 | 4925 | 7892 | 5458 | 646 | 1664 | 1230 | O |
| ATOM | 261 | N | HIS | A | 38 | 78.173 | 49.880 | −3.590 | 1.00 | 45.35 | | N |
| ANISOU | 261 | N | HIS | A | 38 | 4504 | 7474 | 5252 | 359 | 1664 | 1104 | N |
| ATOM | 262 | CA | HIS | A | 38 | 77.211 | 50.979 | −3.736 | 1.00 | 45.68 | | C |
| ANISOU | 262 | CA | HIS | A | 38 | 4784 | 7275 | 5299 | 386 | 1779 | 1196 | C |
| ATOM | 263 | CB | HIS | A | 38 | 76.543 | 51.292 | −2.381 | 1.00 | 45.08 | | C |
| ANISOU | 263 | CB | HIS | A | 38 | 4725 | 7093 | 5310 | 310 | 1728 | 1148 | C |
| ATOM | 264 | CG | HIS | A | 38 | 75.707 | 50.169 | −1.846 | 1.00 | 47.69 | | C |
| ANISOU | 264 | CG | HIS | A | 38 | 4953 | 7496 | 5672 | 459 | 1493 | 1118 | C |
| ATOM | 265 | ND1 | HIS | A | 38 | 74.582 | 49.727 | −2.514 | 1.00 | 48.95 | | N |
| ANISOU | 265 | ND1 | HIS | A | 38 | 5175 | 7634 | 5790 | 691 | 1392 | 1191 | N |
| ATOM | 266 | CE1 | HIS | A | 38 | 74.086 | 48.750 | −1.778 | 1.00 | 48.44 | | C |
| ANISOU | 266 | CE1 | HIS | A | 38 | 5001 | 7643 | 5761 | 732 | 1208 | 1129 | C |
| ATOM | 267 | NE2 | HIS | A | 38 | 74.810 | 48.554 | −0.678 | 1.00 | 48.73 | | N |
| ANISOU | 267 | NE2 | HIS | A | 38 | 4924 | 7736 | 5853 | 568 | 1183 | 1031 | N |
| ATOM | 268 | CD2 | HIS | A | 38 | 75.854 | 49.444 | −0.717 | 1.00 | 49.09 | | C |
| ANISOU | 268 | CD2 | HIS | A | 38 | 4980 | 7768 | 5902 | 395 | 1355 | 1018 | C |
| ATOM | 269 | C | HIS | A | 38 | 77.781 | 52.265 | −4.370 | 1.00 | 53.12 | | C |
| ANISOU | 269 | C | HIS | A | 38 | 5902 | 8086 | 6193 | 249 | 2050 | 1252 | C |
| ATOM | 270 | O | HIS | A | 38 | 77.074 | 53.280 | −4.385 | 1.00 | 53.99 | | O |
| ANISOU | 270 | O | HIS | A | 38 | 6244 | 7974 | 6295 | 268 | 2168 | 1325 | O |
| ATOM | 271 | N | ARG | A | 39 | 79.033 | 52.229 | −4.899 | 1.00 | 50.53 | | N |
| ANISOU | 271 | N | ARG | A | 39 | 5483 | 7894 | 5823 | 122 | 2158 | 1220 | N |
| ATOM | 272 | CA | ARG | A | 39 | 79.674 | 53.407 | −5.496 | 1.00 | 50.69 | | C |
| ANISOU | 272 | CA | ARG | A | 39 | 5674 | 7798 | 5788 | −47 | 2437 | 1263 | C |
| ATOM | 273 | CB | ARG | A | 39 | 81.157 | 53.160 | −5.833 | 1.00 | 48.33 | | C |
| ANISOU | 273 | CB | ARG | A | 39 | 5179 | 7730 | 5452 | −223 | 2523 | 1184 | C |
| ATOM | 274 | CG | ARG | A | 39 | 81.860 | 54.310 | −6.550 | 1.00 | 47.79 | | C |
| ANISOU | 274 | CG | ARG | A | 39 | 5284 | 7557 | 5317 | −435 | 2832 | 1217 | C |
| ATOM | 275 | CD | ARG | A | 39 | 83.357 | 54.139 | −6.558 | 1.00 | 46.02 | | C |
| ANISOU | 275 | CD | ARG | A | 39 | 4807 | 7606 | 5074 | −673 | 2914 | 1097 | C |
| ATOM | 276 | NE | ARG | A | 39 | 83.977 | 54.920 | −5.483 | 1.00 | 61.91 | | N |
| ANISOU | 276 | NE | ARG | A | 39 | 6768 | 9618 | 7137 | −1033 | 3038 | 969 | N |
| ATOM | 277 | CZ | ARG | A | 39 | 84.332 | 54.434 | −4.293 | 1.00 | 76.31 | | C |
| ANISOU | 277 | CZ | ARG | A | 39 | 8335 | 11629 | 9029 | −1135 | 2887 | 828 | C |
| ATOM | 278 | NH1 | ARG | A | 39 | 84.121 | 53.156 | −3.997 | 1.00 | 55.80 | | N |
| ANISOU | 278 | NH1 | ARG | A | 39 | 5528 | 9215 | 6456 | −899 | 2615 | 803 | N |
| ATOM | 279 | NH2 | ARG | A | 39 | 84.895 | 55.225 | −3.388 | 1.00 | 66.69 | | N |
| ANISOU | 279 | NH2 | ARG | A | 39 | 7085 | 10411 | 7841 | −1476 | 3014 | 707 | N |
| ATOM | 280 | C | ARG | A | 39 | 78.894 | 53.939 | −6.688 | 1.00 | 57.98 | | C |
| ANISOU | 280 | C | ARG | A | 39 | 6876 | 8535 | 6620 | 169 | 2530 | 1421 | C |
| ATOM | 281 | O | ARG | A | 39 | 78.588 | 53.199 | −7.631 | 1.00 | 57.20 | | O |
| ANISOU | 281 | O | ARG | A | 39 | 6750 | 8523 | 6460 | 410 | 2419 | 1482 | O |
| ATOM | 282 | N | GLY | A | 40 | 78.556 | 55.220 | −6.595 | 1.00 | 57.80 | | N |
| ANISOU | 282 | N | GLY | A | 40 | 7134 | 8252 | 6575 | 88 | 2734 | 1480 | N |
| ATOM | 283 | CA | GLY | A | 40 | 77.810 | 55.930 | −7.622 | 1.00 | 59.25 | | C |
| ANISOU | 283 | CA | GLY | A | 40 | 7632 | 8229 | 6651 | 301 | 2853 | 1635 | C |
| ATOM | 284 | C | GLY | A | 40 | 76.324 | 56.017 | −7.350 | 1.00 | 66.86 | | C |
| ANISOU | 284 | C | GLY | A | 40 | 8715 | 9068 | 7619 | 561 | 2725 | 1701 | C |
| ATOM | 285 | O | GLY | A | 40 | 75.699 | 57.018 | −7.706 | 1.00 | 68.17 | | O |
| ANISOU | 285 | O | GLY | A | 40 | 9191 | 9000 | 7709 | 669 | 2874 | 1805 | O |
| ATOM | 286 | N | GLN | A | 41 | 75.746 | 54.969 | −6.726 | 1.00 | 63.44 | | N |
| ANISOU | 286 | N | GLN | A | 41 | 8048 | 8795 | 7263 | 670 | 2458 | 1640 | N |
| ATOM | 287 | CA | GLN | A | 41 | 74.319 | 54.883 | −6.421 | 1.00 | 63.18 | | C |
| ANISOU | 287 | CA | GLN | A | 41 | 8061 | 8709 | 7238 | 904 | 2312 | 1678 | C |
| ATOM | 288 | CB | GLN | A | 41 | 73.983 | 53.520 | −5.830 | 1.00 | 64.18 | | C |
| ANISOU | 288 | CB | GLN | A | 41 | 7897 | 9046 | 7444 | 947 | 2036 | 1586 | C |
| ATOM | 289 | CG | GLN | A | 41 | 73.660 | 52.491 | −6.886 | 1.00 | 67.30 | | C |
| ANISOU | 289 | CG | GLN | A | 41 | 8205 | 9599 | 7765 | 1183 | 1877 | 1622 | C |
| ATOM | 290 | CD | GLN | A | 41 | 74.171 | 51.133 | −6.510 | 1.00 | 73.41 | | C |
| ANISOU | 290 | CD | GLN | A | 41 | 8714 | 10583 | 8594 | 1121 | 1692 | 1517 | C |
| ATOM | 291 | OE1 | GLN | A | 41 | 74.033 | 50.675 | −5.363 | 1.00 | 60.05 | | O |
| ANISOU | 291 | OE1 | GLN | A | 41 | 6888 | 8936 | 6993 | 1031 | 1573 | 1428 | O |
| ATOM | 292 | NE2 | GLN | A | 41 | 74.769 | 50.457 | −7.484 | 1.00 | 66.52 | | N |
| ANISOU | 292 | NE2 | GLN | A | 41 | 7785 | 9837 | 7653 | 1185 | 1672 | 1529 | N |
| ATOM | 293 | C | GLN | A | 41 | 73.839 | 55.977 | −5.497 | 1.00 | 69.20 | | C |
| ANISOU | 293 | C | GLN | A | 41 | 9010 | 9249 | 8032 | 826 | 2431 | 1683 | C |
| ATOM | 294 | O | GLN | A | 41 | 74.617 | 56.488 | −4.688 | 1.00 | 69.68 | | O |
| ANISOU | 294 | O | GLN | A | 41 | 9077 | 9243 | 8156 | 538 | 2553 | 1606 | O |
| ATOM | 295 | N | LEU | A | 42 | 72.554 | 56.337 | −5.623 | 1.00 | 66.50 | | N |
| ANISOU | 295 | N | LEU | A | 42 | 8823 | 8809 | 7637 | 1090 | 2397 | 1766 | N |
| ATOM | 296 | CA | LEU | A | 42 | 71.931 | 57.355 | −4.786 | 1.00 | 66.56 | | C |
| ANISOU | 296 | CA | LEU | A | 42 | 9029 | 8604 | 7657 | 1079 | 2502 | 1780 | C |
| ATOM | 297 | CB | LEU | A | 42 | 71.534 | 58.605 | −5.595 | 1.00 | 66.62 | | C |
| ANISOU | 297 | CB | LEU | A | 42 | 9426 | 8368 | 7517 | 1258 | 2724 | 1923 | C |
| ATOM | 298 | CG | LEU | A | 42 | 72.626 | 59.482 | −6.208 | 1.00 | 71.48 | | C |
| ANISOU | 298 | CG | LEU | A | 42 | 10289 | 8806 | 8064 | 1062 | 3010 | 1964 | C |
| ATOM | 299 | CD1 | LEU | A | 42 | 72.066 | 60.834 | −6.558 | 1.00 | 71.73 | | C |
| ANISOU | 299 | CD1 | LEU | A | 42 | 10763 | 8533 | 7956 | 1230 | 3237 | 2092 | C |
| ATOM | 300 | CD2 | LEU | A | 42 | 73.825 | 59.675 | −5.273 | 1.00 | 74.48 | | C |
| ANISOU | 300 | CD2 | LEU | A | 42 | 10585 | 9161 | 8554 | 626 | 3118 | 1833 | C |
| ATOM | 301 | C | LEU | A | 42 | 70.696 | 56.828 | −4.125 | 1.00 | 71.46 | | C |
| ANISOU | 301 | C | LEU | A | 42 | 9517 | 9312 | 8321 | 1259 | 2293 | 1755 | C |
| ATOM | 302 | O | LEU | A | 42 | 70.068 | 55.885 | −4.613 | 1.00 | 71.47 | | O |
| ANISOU | 302 | O | LEU | A | 42 | 9355 | 9502 | 8299 | 1460 | 2098 | 1763 | O |
| ATOM | 303 | N | ARG | A | 43 | 70.328 | 57.480 | −3.024 | 1.00 | 68.34 | | N |
| ANISOU | 303 | N | ARG | A | 43 | 9208 | 8779 | 7979 | 1181 | 2348 | 1721 | N |
| ATOM | 304 | CA | ARG | A | 43 | 69.096 | 57.263 | −2.293 | 1.00 | 68.67 | | C |
| ANISOU | 304 | CA | ARG | A | 43 | 9171 | 8870 | 8049 | 1348 | 2200 | 1703 | C |
| ATOM | 305 | CB | ARG | A | 43 | 69.338 | 57.584 | −0.792 | 1.00 | 68.95 | | C |
| ANISOU | 305 | CB | ARG | A | 43 | 9191 | 8812 | 8197 | 1102 | 2235 | 1604 | C |
| ATOM | 306 | CG | ARG | A | 43 | 69.886 | 56.432 | 0.071 | 1.00 | 72.84 | | C |
| ANISOU | 306 | CG | ARG | A | 43 | 9366 | 9493 | 8816 | 883 | 2059 | 1469 | C |
| ATOM | 307 | CD | ARG | A | 43 | 68.907 | 55.271 | 0.058 | 1.00 | 77.10 | | C |
| ANISOU | 307 | CD | ARG | A | 43 | 9680 | 10244 | 9369 | 1070 | 1814 | 1450 | C |
| ATOM | 308 | NE | ARG | A | 43 | 68.536 | 54.655 | 1.339 | 1.00 | 80.19 | | N |
| ANISOU | 308 | NE | ARG | A | 43 | 9902 | 10711 | 9855 | 984 | 1675 | 1352 | N |
| ATOM | 309 | CZ | ARG | A | 43 | 67.834 | 55.231 | 2.312 | 1.00 | 94.85 | | C |
| ANISOU | 309 | CZ | ARG | A | 43 | 11832 | 12468 | 11739 | 996 | 1707 | 1336 | C |
| ATOM | 310 | NH1 | ARG | A | 43 | 67.538 | 56.525 | 2.254 | 1.00 | 87.66 | | N |
| ANISOU | 310 | NH1 | ARG | A | 43 | 11185 | 11352 | 10772 | 1069 | 1890 | 1406 | N |
| ATOM | 311 | NH2 | ARG | A | 43 | 67.497 | 54.539 | 3.389 | 1.00 | 80.33 | | N |
| ANISOU | 311 | NH2 | ARG | A | 43 | 9827 | 10718 | 9976 | 924 | 1573 | 1248 | N |
| ATOM | 312 | C | ARG | A | 43 | 68.108 | 58.279 | −2.944 | 1.00 | 74.46 | | C |
| ANISOU | 312 | C | ARG | A | 43 | 10192 | 9465 | 8637 | 1677 | 2306 | 1838 | C |
| ATOM | 313 | O | ARG | A | 43 | 68.530 | 58.998 | −3.857 | 1.00 | 75.01 | | O |
| ANISOU | 313 | O | ARG | A | 43 | 10499 | 9403 | 8600 | 1735 | 2478 | 1931 | O |
| ATOM | 314 | N | ARG | A | 44 | 66.829 | 58.362 | −2.492 | 1.00 | 71.86 | | N |
| ANISOU | 314 | N | ARG | A | 44 | 9849 | 9169 | 8286 | 1902 | 2218 | 1848 | N |
| ATOM | 315 | CA | ARG | A | 44 | 65.836 | 59.328 | −3.004 | 1.00 | 72.33 | | C |
| ANISOU | 315 | CA | ARG | A | 44 | 10159 | 9134 | 8191 | 2260 | 2301 | 1967 | C |
| ATOM | 316 | CB | ARG | A | 44 | 64.415 | 59.074 | −2.459 | 1.00 | 75.24 | | C |
| ANISOU | 316 | CB | ARG | A | 44 | 10383 | 9644 | 8560 | 2471 | 2149 | 1937 | C |
| ATOM | 317 | CG | ARG | A | 44 | 63.839 | 57.679 | −2.749 | 1.00 | 94.20 | | C |
| ANISOU | 317 | CG | ARG | A | 44 | 12426 | 12385 | 10980 | 2568 | 1878 | 1878 | C |
| ATOM | 318 | CD | ARG | A | 44 | 63.627 | 57.363 | −4.214 | 1.00 | 111.42 | | C |
| ANISOU | 318 | CD | ARG | A | 44 | 14606 | 14699 | 13028 | 2798 | 1824 | 1952 | C |
| ATOM | 319 | NE | ARG | A | 44 | 62.470 | 58.090 | −4.726 | 1.00 | 122.58 | | N |
| ANISOU | 319 | NE | ARG | A | 44 | 16172 | 16134 | 14270 | 3198 | 1852 | 2047 | N |
| ATOM | 320 | CZ | ARG | A | 44 | 61.254 | 57.581 | −4.851 | 1.00 | 130.74 | | C |
| ANISOU | 320 | CZ | ARG | A | 44 | 16997 | 17440 | 15240 | 3439 | 1671 | 2018 | C |
| ATOM | 321 | NH1 | ARG | A | 44 | 60.262 | 58.327 | −5.313 | 1.00 | 114.73 | | N |
| ANISOU | 321 | NH1 | ARG | A | 44 | 15109 | 15447 | 13036 | 3827 | 1705 | 2103 | N |
| ATOM | 322 | NH2 | ARG | A | 44 | 61.014 | 56.317 | −4.514 | 1.00 | 111.99 | | N |
| ANISOU | 322 | NH2 | ARG | A | 44 | 14277 | 15313 | 12960 | 3295 | 1459 | 1899 | N |
| ATOM | 323 | C | ARG | A | 44 | 66.328 | 60.717 | −2.640 | 1.00 | 77.18 | | C |
| ANISOU | 323 | C | ARG | A | 44 | 11161 | 9407 | 8756 | 2180 | 2584 | 2023 | C |
| ATOM | 324 | O | ARG | A | 44 | 66.280 | 61.623 | −3.478 | 1.00 | 77.56 | | O |
| ANISOU | 324 | O | ARG | A | 44 | 11529 | 9297 | 8646 | 2419 | 2741 | 2147 | O |
| ATOM | 325 | N | SER | A | 45 | 66.935 | 60.815 | −1.458 | 1.00 | 73.68 | | N |
| ANISOU | 325 | N | SER | A | 45 | 10706 | 8851 | 8437 | 1841 | 2653 | 1927 | N |
| ATOM | 326 | CA | SER | A | 45 | 67.595 | 62.070 | −1.023 | 1.00 | 74.02 | | C |
| ANISOU | 326 | CA | SER | A | 45 | 11101 | 8569 | 8452 | 1658 | 2929 | 1944 | C |
| ATOM | 327 | CB | SER | A | 45 | 67.879 | 62.049 | 0.456 | 1.00 | 76.99 | | C |
| ANISOU | 327 | CB | SER | A | 45 | 11391 | 8898 | 8964 | 1373 | 2921 | 1817 | C |
| ATOM | 328 | OG | SER | A | 45 | 68.551 | 60.853 | 0.823 | 1.00 | 84.27 | | O |
| ANISOU | 328 | OG | SER | A | 45 | 11940 | 10052 | 10026 | 1117 | 2745 | 1696 | O |
| ATOM | 329 | C | SER | A | 45 | 68.887 | 62.223 | −1.841 | 1.00 | 81.02 | | C |
| ANISOU | 329 | C | SER | A | 45 | 12031 | 9415 | 9338 | 1398 | 3057 | 1942 | C |
| ATOM | 330 | O | SER | A | 45 | 69.305 | 61.245 | −2.501 | 1.00 | 81.98 | | O |
| ANISOU | 330 | O | SER | A | 45 | 11841 | 9773 | 9534 | 1286 | 2902 | 1883 | O |
| ATOM | 331 | N | GLY | A | 46 | 69.514 | 63.401 | −1.792 | 1.00 | 77.38 | | N |
| ANISOU | 331 | N | GLY | A | 46 | 11958 | 8655 | 8789 | 1288 | 3347 | 1996 | N |
| ATOM | 332 | CA | GLY | A | 46 | 70.748 | 63.682 | −2.556 | 1.00 | 76.55 | | C |
| ANISOU | 332 | CA | GLY | A | 46 | 11931 | 8491 | 8663 | 1020 | 3517 | 1993 | C |
| ATOM | 333 | C | GLY | A | 46 | 71.915 | 62.773 | −2.188 | 1.00 | 78.25 | | C |
| ANISOU | 333 | C | GLY | A | 46 | 11778 | 8924 | 9028 | 644 | 3416 | 1845 | C |
| ATOM | 334 | O | GLY | A | 46 | 72.740 | 62.491 | −3.079 | 1.00 | 78.34 | | O |
| ANISOU | 334 | O | GLY | A | 46 | 11717 | 9027 | 9024 | 516 | 3463 | 1844 | O |
| ATOM | 335 | N | GLU | A | 47 | 71.983 | 62.332 | −0.928 | 1.00 | 71.55 | | N |
| ANISOU | 335 | N | GLU | A | 47 | 10696 | 8177 | 8315 | 493 | 3271 | 1719 | N |
| ATOM | 336 | CA | GLU | A | 47 | 73.093 | 61.497 | −0.394 | 1.00 | 69.29 | | C |
| ANISOU | 336 | CA | GLU | A | 47 | 10067 | 8096 | 8163 | 163 | 3159 | 1564 | C |
| ATOM | 337 | CB | GLU | A | 47 | 72.898 | 61.263 | 1.105 | 1.00 | 70.99 | | C |
| ANISOU | 337 | CB | GLU | A | 47 | 10134 | 8355 | 8485 | 111 | 3015 | 1464 | C |
| ATOM | 338 | CG | GLU | A | 47 | 72.156 | 62.390 | 1.801 | 1.00 | 87.79 | | C |
| ANISOU | 338 | CG | GLU | A | 47 | 12584 | 10189 | 10585 | 68 | 3191 | 1466 | C |
| ATOM | 339 | CD | GLU | A | 47 | 73.052 | 63.427 | 2.456 | 1.00 | 121.67 | | C |
| ANISOU | 339 | CD | GLU | A | 47 | 17050 | 14294 | 14884 | −338 | 3420 | 1369 | C |
| ATOM | 340 | OE1 | GLU | A | 47 | 74.133 | 63.046 | 2.945 | 1.00 | 117.22 | | O |
| ANISOU | 340 | OE1 | GLU | A | 47 | 16234 | 13905 | 14398 | −655 | 3378 | 1238 | O |
| ATOM | 341 | OE2 | GLU | A | 47 | 72.666 | 64.612 | 2.474 | 1.00 | 124.64 | | O |
| ANISOU | 341 | OE2 | GLU | A | 47 | 17822 | 14357 | 15179 | −333 | 3644 | 1417 | O |
| ATOM | 342 | C | GLU | A | 47 | 73.252 | 60.156 | −1.129 | 1.00 | 67.52 | | C |
| ANISOU | 342 | C | GLU | A | 47 | 9505 | 8178 | 7970 | 214 | 2963 | 1547 | C |
| ATOM | 343 | O | GLU | A | 47 | 72.249 | 59.556 | −1.562 | 1.00 | 66.15 | | O |
| ANISOU | 343 | O | GLU | A | 47 | 9229 | 8130 | 7775 | 521 | 2783 | 1610 | O |
| ATOM | 344 | N | PRO | A | 48 | 74.520 | 59.705 | −1.256 | 1.00 | 60.67 | | N |
| ANISOU | 344 | N | PRO | A | 48 | 8468 | 7445 | 7140 | −77 | 3001 | 1458 | N |
| ATOM | 345 | CA | PRO | A | 48 | 74.942 | 58.434 | −1.877 | 1.00 | 58.92 | | C |
| ANISOU | 345 | CA | PRO | A | 48 | 7935 | 7515 | 6938 | −6 | 2813 | 1439 | C |
| ATOM | 346 | CB | PRO | A | 48 | 76.464 | 58.499 | −1.818 | 1.00 | 60.78 | | C |
| ANISOU | 346 | CB | PRO | A | 48 | 8050 | 7852 | 7192 | −359 | 2931 | 1335 | C |
| ATOM | 347 | CG | PRO | A | 48 | 76.765 | 59.408 | −0.672 | 1.00 | 65.48 | | C |
| ANISOU | 347 | CG | PRO | A | 48 | 8755 | 8295 | 7830 | −652 | 3069 | 1241 | C |
| ATOM | 348 | CD | PRO | A | 48 | 75.671 | 60.427 | −0.672 | 1.00 | 61.48 | | C |
| ANISOU | 348 | CD | PRO | A | 48 | 8628 | 7470 | 7262 | −484 | 3203 | 1354 | C |
| ATOM | 349 | C | PRO | A | 48 | 74.409 | 57.255 | −1.073 | 1.00 | 59.20 | | C |
| ANISOU | 349 | C | PRO | A | 48 | 7676 | 7752 | 7068 | 87 | 2526 | 1366 | C |
| ATOM | 350 | O | PRO | A | 48 | 74.269 | 57.368 | 0.150 | 1.00 | 59.75 | | O |
| ANISOU | 350 | O | PRO | A | 48 | 7698 | 7793 | 7212 | −41 | 2487 | 1281 | O |
| ATOM | 351 | N | TYR | A | 49 | 74.083 | 56.147 | −1.742 | 1.00 | 51.12 | | N |
| ANISOU | 351 | N | TYR | A | 49 | 6479 | 6915 | 6029 | 303 | 2336 | 1398 | N |
| ATOM | 352 | CA | TYR | A | 49 | 73.450 | 55.006 | −1.084 | 1.00 | 48.79 | | C |
| ANISOU | 352 | CA | TYR | A | 49 | 5951 | 6785 | 5802 | 406 | 2078 | 1339 | C |
| ATOM | 353 | CB | TYR | A | 49 | 73.238 | 53.855 | −2.071 | 1.00 | 49.12 | | C |
| ANISOU | 353 | CB | TYR | A | 49 | 5850 | 7014 | 5799 | 612 | 1912 | 1374 | C |
| ATOM | 354 | CG | TYR | A | 49 | 72.143 | 52.895 | −1.655 | 1.00 | 48.95 | | C |
| ANISOU | 354 | CG | TYR | A | 49 | 5694 | 7097 | 5808 | 780 | 1683 | 1352 | C |
| ATOM | 355 | CD1 | TYR | A | 49 | 70.832 | 53.092 | −2.057 | 1.00 | 50.53 | | C |
| ANISOU | 355 | CD1 | TYR | A | 49 | 5993 | 7252 | 5954 | 1025 | 1644 | 1430 | C |
| ATOM | 356 | CE1 | TYR | A | 49 | 69.826 | 52.196 | −1.706 | 1.00 | 51.00 | | C |
| ANISOU | 356 | CE1 | TYR | A | 49 | 5913 | 7431 | 6035 | 1145 | 1446 | 1394 | C |
| ATOM | 357 | CZ | TYR | A | 49 | 70.125 | 51.090 | −0.936 | 1.00 | 56.31 | | C |
| ANISOU | 357 | CZ | TYR | A | 49 | 6386 | 8229 | 6779 | 1023 | 1295 | 1289 | C |
| ATOM | 358 | OH | TYR | A | 49 | 69.110 | 50.240 | −0.568 | 1.00 | 59.77 | | O |
| ANISOU | 358 | OH | TYR | A | 49 | 6715 | 8764 | 7230 | 1110 | 1124 | 1249 | O |
| ATOM | 359 | CE2 | TYR | A | 49 | 71.426 | 50.869 | −0.526 | 1.00 | 49.69 | | C |
| ANISOU | 359 | CE2 | TYR | A | 49 | 5467 | 7426 | 5987 | 817 | 1324 | 1221 | C |
| ATOM | 360 | CD2 | TYR | A | 49 | 72.428 | 51.761 | −0.899 | 1.00 | 49.20 | | C |
| ANISOU | 360 | CD2 | TYR | A | 49 | 5504 | 7285 | 5906 | 696 | 1512 | 1248 | C |
| ATOM | 361 | C | TYR | A | 49 | 74.166 | 54.512 | 0.179 | 1.00 | 50.21 | | C |
| ANISOU | 361 | C | TYR | A | 49 | 5923 | 7077 | 6079 | 166 | 1996 | 1196 | C |
| ATOM | 362 | O | TYR | A | 49 | 73.496 | 54.168 | 1.156 | 1.00 | 48.36 | | O |
| ANISOU | 362 | O | TYR | A | 49 | 5621 | 6852 | 5903 | 193 | 1871 | 1150 | O |
| ATOM | 363 | N | ILE | A | 50 | 75.516 | 54.521 | 0.183 | 1.00 | 46.63 | | N |
| ANISOU | 363 | N | ILE | A | 50 | 5368 | 6718 | 5630 | −65 | 2073 | 1122 | N |
| ATOM | 364 | CA | ILE | A | 50 | 76.332 | 54.034 | 1.311 | 1.00 | 45.97 | | C |
| ANISOU | 364 | CA | ILE | A | 50 | 5067 | 6788 | 5612 | −274 | 1991 | 978 | C |
| ATOM | 365 | CB | ILE | A | 50 | 77.867 | 54.240 | 1.092 | 1.00 | 48.73 | | C |
| ANISOU | 365 | CB | ILE | A | 50 | 5310 | 7268 | 5937 | −525 | 2116 | 900 | C |
| ATOM | 366 | CG1 | ILE | A | 50 | 78.734 | 53.705 | 2.269 | 1.00 | 48.32 | | C |
| ANISOU | 366 | CG1 | ILE | A | 50 | 5001 | 7428 | 5930 | −706 | 2009 | 741 | C |
| ATOM | 367 | CD1 | ILE | A | 50 | 78.781 | 52.203 | 2.423 | 1.00 | 49.68 | | C |
| ANISOU | 367 | CD1 | ILE | A | 50 | 4946 | 7826 | 6104 | −533 | 1767 | 707 | C |
| ATOM | 368 | CG2 | ILE | A | 50 | 78.214 | 55.684 | 0.768 | 1.00 | 50.40 | | C |
| ANISOU | 368 | CG2 | ILE | A | 50 | 5763 | 7273 | 6114 | −724 | 2398 | 926 | C |
| ATOM | 369 | C | ILE | A | 50 | 75.853 | 54.578 | 2.663 | 1.00 | 49.27 | | C |
| ANISOU | 369 | C | ILE | A | 50 | 5549 | 7080 | 6092 | −382 | 1997 | 920 | C |
| ATOM | 370 | O | ILE | A | 50 | 76.026 | 53.883 | 3.665 | 1.00 | 50.25 | | O |
| ANISOU | 370 | O | ILE | A | 50 | 5494 | 7332 | 6266 | −444 | 1853 | 821 | O |
| ATOM | 371 | N | THR | A | 51 | 75.204 | 55.769 | 2.685 | 1.00 | 43.70 | | N |
| ANISOU | 371 | N | THR | A | 51 | 5115 | 6120 | 5371 | −377 | 2159 | 987 | N |
| ATOM | 372 | CA | THR | A | 51 | 74.684 | 56.372 | 3.921 | 1.00 | 42.48 | | C |
| ANISOU | 372 | CA | THR | A | 51 | 5058 | 5821 | 5262 | −459 | 2184 | 940 | C |
| ATOM | 373 | CB | THR | A | 51 | 74.170 | 57.799 | 3.708 | 1.00 | 44.39 | | C |
| ANISOU | 373 | CB | THR | A | 51 | 5649 | 5764 | 5453 | −445 | 2412 | 1024 | C |
| ATOM | 374 | OG1 | THR | A | 51 | 72.959 | 57.773 | 2.964 | 1.00 | 42.74 | | O |
| ANISOU | 374 | OG1 | THR | A | 51 | 5555 | 5488 | 5196 | −111 | 2367 | 1156 | O |
| ATOM | 375 | CG2 | THR | A | 51 | 75.193 | 58.704 | 3.049 | 1.00 | 41.27 | | C |
| ANISOU | 375 | CG2 | THR | A | 51 | 5413 | 5271 | 4998 | −654 | 2656 | 1032 | C |
| ATOM | 376 | C | THR | A | 51 | 73.655 | 55.459 | 4.620 | 1.00 | 43.94 | | C |
| ANISOU | 376 | C | THR | A | 51 | 5118 | 6082 | 5497 | −277 | 1962 | 930 | C |
| ATOM | 377 | O | THR | A | 51 | 73.667 | 55.374 | 5.852 | 1.00 | 42.34 | | O |
| ANISOU | 377 | O | THR | A | 51 | 4849 | 5891 | 5347 | −395 | 1905 | 838 | O |
| ATOM | 378 | N | HIS | A | 52 | 72.818 | 54.745 | 3.821 | 1.00 | 39.40 | | N |
| ANISOU | 378 | N | HIS | A | 52 | 4505 | 5572 | 4894 | −9 | 1840 | 1015 | N |
| ATOM | 379 | CA | HIS | A | 52 | 71.804 | 53.807 | 4.306 | 1.00 | 38.60 | | C |
| ANISOU | 379 | CA | HIS | A | 52 | 4284 | 5559 | 4824 | 150 | 1641 | 1005 | C |
| ATOM | 380 | CB | HIS | A | 52 | 70.814 | 53.407 | 3.190 | 1.00 | 38.31 | | C |
| ANISOU | 380 | CB | HIS | A | 52 | 4268 | 5559 | 4727 | 432 | 1570 | 1108 | C |
| ATOM | 381 | CG | HIS | A | 52 | 69.887 | 52.268 | 3.522 | 1.00 | 41.43 | | C |
| ANISOU | 381 | CG | HIS | A | 52 | 4510 | 6089 | 5143 | 557 | 1364 | 1079 | C |
| ATOM | 382 | ND1 | HIS | A | 52 | 69.017 | 52.326 | 4.601 | 1.00 | 42.30 | | N |
| ANISOU | 382 | ND1 | HIS | A | 52 | 4616 | 6162 | 5296 | 561 | 1318 | 1043 | N |
| ATOM | 383 | CE1 | HIS | A | 52 | 68.337 | 51.190 | 4.579 | 1.00 | 41.30 | | C |
| ANISOU | 383 | CE1 | HIS | A | 52 | 4349 | 6175 | 5166 | 656 | 1147 | 1020 | C |
| ATOM | 384 | NE2 | HIS | A | 52 | 68.706 | 50.418 | 3.559 | 1.00 | 41.82 | | N |
| ANISOU | 384 | NE2 | HIS | A | 52 | 4344 | 6356 | 5189 | 718 | 1078 | 1038 | N |
| ATOM | 385 | CD2 | HIS | A | 52 | 69.688 | 51.098 | 2.870 | 1.00 | 42.27 | | C |
| ANISOU | 385 | CD2 | HIS | A | 52 | 4486 | 6355 | 5221 | 668 | 1213 | 1080 | C |
| ATOM | 386 | C | HIS | A | 52 | 72.429 | 52.586 | 5.010 | 1.00 | 42.85 | | C |
| ANISOU | 386 | C | HIS | A | 52 | 4584 | 6293 | 5406 | 54 | 1469 | 897 | C |
| ATOM | 387 | O | HIS | A | 52 | 72.171 | 52.437 | 6.204 | 1.00 | 43.22 | | O |
| ANISOU | 387 | O | HIS | A | 52 | 4590 | 6332 | 5501 | −16 | 1407 | 828 | O |
| ATOM | 388 | N | PRO | A | 53 | 73.253 | 51.712 | 4.376 | 1.00 | 39.01 | | N |
| ANISOU | 388 | N | PRO | A | 53 | 3952 | 5975 | 4893 | 59 | 1394 | 878 | N |
| ATOM | 389 | CA | PRO | A | 53 | 73.784 | 50.566 | 5.128 | 1.00 | 38.73 | | C |
| ANISOU | 389 | CA | PRO | A | 53 | 3727 | 6111 | 4878 | 7 | 1235 | 779 | C |
| ATOM | 390 | CB | PRO | A | 53 | 74.542 | 49.770 | 4.067 | 1.00 | 40.69 | | C |
| ANISOU | 390 | CB | PRO | A | 53 | 3873 | 6517 | 5071 | 71 | 1194 | 791 | C |
| ATOM | 391 | CG | PRO | A | 53 | 73.938 | 50.194 | 2.749 | 1.00 | 45.42 | | C |
| ANISOU | 391 | CG | PRO | A | 53 | 4598 | 7035 | 5626 | 221 | 1266 | 908 | C |
| ATOM | 392 | CD | PRO | A | 53 | 73.687 | 51.653 | 2.963 | 1.00 | 40.87 | | C |
| ANISOU | 392 | CD | PRO | A | 53 | 4204 | 6258 | 5066 | 141 | 1447 | 948 | C |
| ATOM | 393 | C | PRO | A | 53 | 74.643 | 50.946 | 6.336 | 1.00 | 43.70 | | C |
| ANISOU | 393 | C | PRO | A | 53 | 4301 | 6762 | 5542 | −219 | 1272 | 668 | C |
| ATOM | 394 | O | PRO | A | 53 | 74.685 | 50.181 | 7.314 | 1.00 | 44.21 | | O |
| ANISOU | 394 | O | PRO | A | 53 | 4264 | 6911 | 5623 | −233 | 1140 | 592 | O |
| ATOM | 395 | N | VAL | A | 54 | 75.287 | 52.144 | 6.297 | 1.00 | 39.93 | | N |
| ANISOU | 395 | N | VAL | A | 54 | 3905 | 6202 | 5063 | −402 | 1457 | 653 | N |
| ATOM | 396 | CA | VAL | A | 54 | 76.094 | 52.666 | 7.414 | 1.00 | 39.34 | | C |
| ANISOU | 396 | CA | VAL | A | 54 | 3787 | 6148 | 5010 | −651 | 1514 | 533 | C |
| ATOM | 397 | CB | VAL | A | 54 | 76.984 | 53.865 | 6.986 | 1.00 | 41.84 | | C |
| ANISOU | 397 | CB | VAL | A | 54 | 4200 | 6394 | 5304 | −873 | 1741 | 518 | C |
| ATOM | 398 | CG1 | VAL | A | 54 | 77.600 | 54.561 | 8.199 | 1.00 | 41.59 | | C |
| ANISOU | 398 | CG1 | VAL | A | 54 | 4174 | 6336 | 5292 | −1149 | 1820 | 391 | C |
| ATOM | 399 | CG2 | VAL | A | 54 | 78.075 | 53.420 | 6.016 | 1.00 | 40.89 | | C |
| ANISOU | 399 | CG2 | VAL | A | 54 | 3928 | 6474 | 5135 | −906 | 1759 | 504 | C |
| ATOM | 400 | C | VAL | A | 54 | 75.137 | 52.992 | 8.599 | 1.00 | 45.48 | | C |
| ANISOU | 400 | C | VAL | A | 54 | 4663 | 6782 | 5835 | −654 | 1487 | 514 | C |
| ATOM | 401 | O | VAL | A | 54 | 75.383 | 52.558 | 9.731 | 1.00 | 47.06 | | O |
| ANISOU | 401 | O | VAL | A | 54 | 4756 | 7072 | 6051 | −730 | 1388 | 416 | O |
| ATOM | 402 | N | ALA | A | 55 | 74.010 | 53.686 | 8.314 | 1.00 | 40.74 | | N |
| ANISOU | 402 | N | ALA | A | 55 | 4262 | 5975 | 5242 | −541 | 1568 | 611 | N |
| ATOM | 403 | CA | ALA | A | 55 | 72.965 | 54.006 | 9.293 | 1.00 | 39.04 | | C |
| ANISOU | 403 | CA | ALA | A | 55 | 4148 | 5625 | 5061 | −502 | 1556 | 609 | C |
| ATOM | 404 | CB | ALA | A | 55 | 71.883 | 54.849 | 8.644 | 1.00 | 39.43 | | C |
| ANISOU | 404 | CB | ALA | A | 55 | 4415 | 5479 | 5089 | −340 | 1671 | 729 | C |
| ATOM | 405 | C | ALA | A | 55 | 72.362 | 52.721 | 9.880 | 1.00 | 41.04 | | C |
| ANISOU | 405 | C | ALA | A | 55 | 4257 | 6001 | 5335 | −378 | 1348 | 585 | C |
| ATOM | 406 | O | ALA | A | 55 | 72.170 | 52.653 | 11.096 | 1.00 | 41.45 | | O |
| ANISOU | 406 | O | ALA | A | 55 | 4297 | 6037 | 5414 | −447 | 1303 | 515 | O |
| ATOM | 407 | N | VAL | A | 56 | 72.118 | 51.691 | 9.029 | 1.00 | 35.73 | | N |
| ANISOU | 407 | N | VAL | A | 56 | 3490 | 5445 | 4639 | −211 | 1230 | 635 | N |
| ATOM | 408 | CA | VAL | A | 56 | 71.609 | 50.392 | 9.473 | 1.00 | 35.21 | | C |
| ANISOU | 408 | CA | VAL | A | 56 | 3314 | 5486 | 4576 | −113 | 1048 | 608 | C |
| ATOM | 409 | CB | VAL | A | 56 | 71.209 | 49.469 | 8.300 | 1.00 | 38.32 | | C |
| ANISOU | 409 | CB | VAL | A | 56 | 3661 | 5969 | 4932 | 67 | 955 | 674 | C |
| ATOM | 410 | CG1 | VAL | A | 56 | 70.711 | 48.115 | 8.810 | 1.00 | 38.22 | | C |
| ANISOU | 410 | CG1 | VAL | A | 56 | 3571 | 6042 | 4910 | 130 | 787 | 633 | C |
| ATOM | 411 | CG2 | VAL | A | 56 | 70.149 | 50.123 | 7.432 | 1.00 | 37.61 | | C |
| ANISOU | 411 | CG2 | VAL | A | 56 | 3680 | 5783 | 4827 | 212 | 1028 | 775 | C |
| ATOM | 412 | C | VAL | A | 56 | 72.639 | 49.744 | 10.418 | 1.00 | 40.24 | | C |
| ANISOU | 412 | C | VAL | A | 56 | 3827 | 6252 | 5209 | −238 | 962 | 497 | C |
| ATOM | 413 | O | VAL | A | 56 | 72.252 | 49.262 | 11.489 | 1.00 | 39.97 | | O |
| ANISOU | 413 | O | VAL | A | 56 | 3779 | 6222 | 5187 | −249 | 879 | 445 | O |
| ATOM | 414 | N | ALA | A | 57 | 73.950 | 49.813 | 10.062 | 1.00 | 37.04 | | N |
| ANISOU | 414 | N | ALA | A | 57 | 3336 | 5960 | 4776 | −333 | 995 | 456 | N |
| ATOM | 415 | CA | ALA | A | 57 | 75.039 | 49.303 | 10.898 | 1.00 | 36.98 | | C |
| ANISOU | 415 | CA | ALA | A | 57 | 3190 | 6118 | 4741 | −434 | 921 | 342 | C |
| ATOM | 416 | CB | ALA | A | 57 | 76.353 | 49.415 | 10.173 | 1.00 | 37.44 | | C |
| ANISOU | 416 | CB | ALA | A | 57 | 3138 | 6328 | 4760 | −509 | 976 | 312 | C |
| ATOM | 417 | C | ALA | A | 57 | 75.104 | 50.062 | 12.226 | 1.00 | 44.10 | | C |
| ANISOU | 417 | C | ALA | A | 57 | 4130 | 6954 | 5671 | −606 | 971 | 259 | C |
| ATOM | 418 | O | ALA | A | 57 | 75.336 | 49.446 | 13.268 | 1.00 | 44.87 | | O |
| ANISOU | 418 | O | ALA | A | 57 | 4154 | 7148 | 5747 | −624 | 864 | 176 | O |
| ATOM | 419 | N | GLU | A | 58 | 74.845 | 51.383 | 12.207 | 1.00 | 41.37 | | N |
| ANISOU | 419 | N | GLU | A | 58 | 3927 | 6432 | 5361 | −717 | 1134 | 282 | N |
| ATOM | 420 | CA | GLU | A | 58 | 74.838 | 52.201 | 13.423 | 1.00 | 41.20 | | C |
| ANISOU | 420 | CA | GLU | A | 58 | 3974 | 6319 | 5360 | −884 | 1196 | 202 | C |
| ATOM | 421 | CB | GLU | A | 58 | 74.709 | 53.688 | 13.089 | 1.00 | 43.24 | | C |
| ANISOU | 421 | CB | GLU | A | 58 | 4424 | 6373 | 5635 | −1008 | 1409 | 233 | C |
| ATOM | 422 | CG | GLU | A | 58 | 76.013 | 54.447 | 13.262 | 1.00 | 59.92 | | C |
| ANISOU | 422 | CG | GLU | A | 58 | 6496 | 8549 | 7721 | −1284 | 1523 | 124 | C |
| ATOM | 423 | CD | GLU | A | 58 | 75.951 | 55.917 | 12.895 | 1.00 | 88.40 | | C |
| ANISOU | 423 | CD | GLU | A | 58 | 10339 | 11923 | 11328 | −1431 | 1760 | 152 | C |
| ATOM | 424 | OE1 | GLU | A | 58 | 75.655 | 56.216 | 11.716 | 1.00 | 77.72 | | O |
| ANISOU | 424 | OE1 | GLU | A | 58 | 9094 | 10472 | 9962 | −1334 | 1857 | 264 | O |
| ATOM | 425 | OE2 | GLU | A | 58 | 76.201 | 56.768 | 13.782 | 1.00 | 85.26 | | O |
| ANISOU | 425 | OE2 | GLU | A | 58 | 10036 | 11430 | 10927 | −1638 | 1854 | 60 | O |
| ATOM | 426 | C | GLU | A | 58 | 73.741 | 51.777 | 14.383 | 1.00 | 42.60 | | C |
| ANISOU | 426 | C | GLU | A | 58 | 4203 | 6424 | 5560 | −782 | 1103 | 208 | C |
| ATOM | 427 | O | GLU | A | 58 | 73.981 | 51.747 | 15.589 | 1.00 | 42.31 | | O |
| ANISOU | 427 | O | GLU | A | 58 | 4141 | 6418 | 5516 | −879 | 1061 | 114 | O |
| ATOM | 428 | N | ILE | A | 59 | 72.543 | 51.438 | 13.854 | 1.00 | 37.84 | | N |
| ANISOU | 428 | N | ILE | A | 59 | 3662 | 5740 | 4974 | −594 | 1073 | 310 | N |
| ATOM | 429 | CA | ILE | A | 59 | 71.396 | 50.975 | 14.659 | 1.00 | 36.61 | | C |
| ANISOU | 429 | CA | ILE | A | 59 | 3543 | 5533 | 4835 | −501 | 997 | 318 | C |
| ATOM | 430 | CB | ILE | A | 59 | 70.103 | 50.851 | 13.794 | 1.00 | 39.17 | | C |
| ANISOU | 430 | CB | ILE | A | 59 | 3921 | 5793 | 5168 | −313 | 1000 | 428 | C |
| ATOM | 431 | CG1 | ILE | A | 59 | 69.637 | 52.210 | 13.282 | 1.00 | 39.07 | | C |
| ANISOU | 431 | CG1 | ILE | A | 59 | 4064 | 5616 | 5166 | −292 | 1168 | 495 | C |
| ATOM | 432 | CD1 | ILE | A | 59 | 68.947 | 52.118 | 12.016 | 1.00 | 48.52 | | C |
| ANISOU | 432 | CD1 | ILE | A | 59 | 5280 | 6814 | 6342 | −110 | 1176 | 598 | C |
| ATOM | 433 | CG2 | ILE | A | 59 | 68.976 | 50.154 | 14.545 | 1.00 | 39.55 | | C |
| ANISOU | 433 | CG2 | ILE | A | 59 | 3961 | 5846 | 5223 | −234 | 909 | 421 | C |
| ATOM | 434 | C | ILE | A | 59 | 71.764 | 49.657 | 15.348 | 1.00 | 38.27 | | C |
| ANISOU | 434 | C | ILE | A | 59 | 3637 | 5891 | 5011 | −483 | 833 | 251 | C |
| ATOM | 435 | O | ILE | A | 59 | 71.527 | 49.513 | 16.536 | 1.00 | 36.03 | | O |
| ANISOU | 435 | O | ILE | A | 59 | 3376 | 5588 | 4726 | −526 | 796 | 194 | O |
| ATOM | 436 | N | LEU | A | 60 | 72.381 | 48.727 | 14.596 | 1.00 | 36.21 | | N |
| ANISOU | 436 | N | LEU | A | 60 | 3274 | 5772 | 4711 | −409 | 745 | 260 | N |
| ATOM | 437 | CA | LEU | A | 60 | 72.806 | 47.417 | 15.082 | 1.00 | 36.38 | | C |
| ANISOU | 437 | CA | LEU | A | 60 | 3220 | 5928 | 4675 | −351 | 597 | 208 | C |
| ATOM | 438 | CB | LEU | A | 60 | 73.115 | 46.447 | 13.919 | 1.00 | 35.68 | | C |
| ANISOU | 438 | CB | LEU | A | 60 | 3070 | 5947 | 4541 | −220 | 525 | 251 | C |
| ATOM | 439 | CG | LEU | A | 60 | 72.003 | 46.207 | 12.867 | 1.00 | 38.89 | | C |
| ANISOU | 439 | CG | LEU | A | 60 | 3532 | 6278 | 4967 | −101 | 531 | 349 | C |
| ATOM | 440 | CD1 | LEU | A | 60 | 72.575 | 45.691 | 11.583 | 1.00 | 38.59 | | C |
| ANISOU | 440 | CD1 | LEU | A | 60 | 3437 | 6338 | 4887 | −12 | 506 | 386 | C |
| ATOM | 441 | CD2 | LEU | A | 60 | 70.989 | 45.208 | 13.339 | 1.00 | 40.34 | | C |
| ANISOU | 441 | CD2 | LEU | A | 60 | 3767 | 6427 | 5132 | −29 | 440 | 352 | C |
| ATOM | 442 | C | LEU | A | 60 | 73.968 | 47.516 | 16.087 | 1.00 | 44.73 | | C |
| ANISOU | 442 | C | LEU | A | 60 | 4202 | 7100 | 5692 | −467 | 570 | 94 | C |
| ATOM | 443 | O | LEU | A | 60 | 73.956 | 46.794 | 17.074 | 1.00 | 44.93 | | O |
| ANISOU | 443 | O | LEU | A | 60 | 4234 | 7165 | 5674 | −437 | 476 | 43 | O |
| ATOM | 444 | N | ALA | A | 61 | 74.923 | 48.445 | 15.891 | 1.00 | 44.67 | | N |
| ANISOU | 444 | N | ALA | A | 61 | 4135 | 7147 | 5690 | −611 | 663 | 48 | N |
| ATOM | 445 | CA | ALA | A | 61 | 76.036 | 48.639 | 16.841 | 1.00 | 46.06 | | C |
| ANISOU | 445 | CA | ALA | A | 61 | 4212 | 7468 | 5819 | −749 | 642 | −81 | C |
| ATOM | 446 | CB | ALA | A | 61 | 77.153 | 49.452 | 16.207 | 1.00 | 46.95 | | C |
| ANISOU | 446 | CB | ALA | A | 61 | 4230 | 7681 | 5927 | −909 | 749 | −127 | C |
| ATOM | 447 | C | ALA | A | 61 | 75.572 | 49.300 | 18.146 | 1.00 | 53.20 | | C |
| ANISOU | 447 | C | ALA | A | 61 | 5213 | 8254 | 6747 | −863 | 678 | −137 | C |
| ATOM | 448 | O | ALA | A | 61 | 76.148 | 49.035 | 19.212 | 1.00 | 54.07 | | O |
| ANISOU | 448 | O | ALA | A | 61 | 5262 | 8484 | 6798 | −907 | 600 | −240 | O |
| ATOM | 449 | N | GLY | A | 62 | 74.540 | 50.145 | 18.046 | 1.00 | 50.61 | | N |
| ANISOU | 449 | N | GLY | A | 62 | 5038 | 7702 | 6489 | −889 | 791 | −70 | N |
| ATOM | 450 | CA | GLY | A | 62 | 73.927 | 50.809 | 19.193 | 1.00 | 50.92 | | C |
| ANISOU | 450 | CA | GLY | A | 62 | 5196 | 7601 | 6551 | −971 | 840 | −108 | C |
| ATOM | 451 | C | GLY | A | 62 | 73.230 | 49.790 | 20.078 | 1.00 | 55.15 | | C |
| ANISOU | 451 | C | GLY | A | 62 | 5751 | 8144 | 7061 | −847 | 714 | −108 | C |
| ATOM | 452 | O | GLY | A | 62 | 73.238 | 49.898 | 21.315 | 1.00 | 54.68 | | O |
| ANISOU | 452 | O | GLY | A | 62 | 5723 | 8078 | 6974 | −911 | 690 | −184 | O |
| ATOM | 453 | N | LEU | A | 63 | 72.696 | 48.743 | 19.422 | 1.00 | 51.11 | | N |
| ANISOU | 453 | N | LEU | A | 63 | 5223 | 7654 | 6544 | −679 | 635 | −28 | N |
| ATOM | 454 | CA | LEU | A | 63 | 72.014 | 47.600 | 20.016 | 1.00 | 50.71 | | C |
| ANISOU | 454 | CA | LEU | A | 63 | 5208 | 7603 | 6456 | −563 | 528 | −17 | C |
| ATOM | 455 | CB | LEU | A | 63 | 71.045 | 47.023 | 18.977 | 1.00 | 50.73 | | C |
| ANISOU | 455 | CB | LEU | A | 63 | 5235 | 7554 | 6485 | −434 | 519 | 86 | C |
| ATOM | 456 | CG | LEU | A | 63 | 69.595 | 47.507 | 19.014 | 1.00 | 55.26 | | C |
| ANISOU | 456 | CG | LEU | A | 63 | 5898 | 7983 | 7117 | −413 | 595 | 142 | C |
| ATOM | 457 | CD1 | LEU | A | 63 | 69.470 | 49.054 | 18.988 | 1.00 | 55.62 | | C |
| ANISOU | 457 | CD1 | LEU | A | 63 | 6000 | 7916 | 7218 | −492 | 740 | 154 | C |
| ATOM | 458 | CD2 | LEU | A | 63 | 68.830 | 46.921 | 17.877 | 1.00 | 56.28 | | C |
| ANISOU | 458 | CD2 | LEU | A | 63 | 6011 | 8121 | 7252 | −296 | 569 | 221 | C |
| ATOM | 459 | C | LEU | A | 63 | 73.015 | 46.521 | 20.491 | 1.00 | 54.00 | | C |
| ANISOU | 459 | C | LEU | A | 63 | 5550 | 8195 | 6772 | −508 | 393 | −84 | C |
| ATOM | 460 | O | LEU | A | 63 | 72.601 | 45.525 | 21.098 | 1.00 | 54.52 | | O |
| ANISOU | 460 | O | LEU | A | 63 | 5680 | 8251 | 6783 | −415 | 309 | −82 | O |
| ATOM | 461 | N | GLN | A | 64 | 74.331 | 46.755 | 20.252 | 1.00 | 48.46 | | N |
| ANISOU | 461 | N | GLN | A | 64 | 4721 | 7658 | 6032 | −564 | 381 | −150 | N |
| ATOM | 462 | CA | GLN | A | 64 | 75.457 | 45.901 | 20.644 | 1.00 | 47.82 | | C |
| ANISOU | 462 | CA | GLN | A | 64 | 4540 | 7793 | 5837 | −491 | 257 | −225 | C |
| ATOM | 463 | CB | GLN | A | 64 | 75.688 | 45.885 | 22.173 | 1.00 | 49.20 | | C |
| ANISOU | 463 | CB | GLN | A | 64 | 4741 | 8010 | 5943 | −530 | 202 | −322 | C |
| ATOM | 464 | CG | GLN | A | 64 | 76.629 | 47.004 | 22.632 | 1.00 | 62.92 | | C |
| ANISOU | 464 | CG | GLN | A | 64 | 6368 | 9862 | 7677 | −726 | 253 | −439 | C |
| ATOM | 465 | CD | GLN | A | 64 | 77.260 | 46.809 | 23.994 | 1.00 | 78.22 | | C |
| ANISOU | 465 | CD | GLN | A | 64 | 8274 | 11941 | 9506 | −736 | 162 | −559 | C |
| ATOM | 466 | OE1 | GLN | A | 64 | 77.275 | 45.711 | 24.573 | 1.00 | 74.05 | | O |
| ANISOU | 466 | OE1 | GLN | A | 64 | 7791 | 11470 | 8876 | −559 | 43 | −558 | O |
| ATOM | 467 | NE2 | GLN | A | 64 | 77.820 | 47.889 | 24.527 | 1.00 | 66.44 | | N |
| ANISOU | 467 | NE2 | GLN | A | 64 | 6719 | 10507 | 8018 | −945 | 222 | −669 | N |
| ATOM | 468 | C | GLN | A | 64 | 75.338 | 44.508 | 20.048 | 1.00 | 51.29 | | C |
| ANISOU | 468 | C | GLN | A | 64 | 5005 | 8269 | 6214 | −287 | 159 | −163 | C |
| ATOM | 469 | O | GLN | A | 64 | 75.450 | 43.492 | 20.733 | 1.00 | 50.35 | | O |
| ANISOU | 469 | O | GLN | A | 64 | 4944 | 8193 | 5995 | −165 | 57 | −186 | O |
| ATOM | 470 | N | MET | A | 65 | 75.138 | 44.486 | 18.737 | 1.00 | 48.51 | | N |
| ANISOU | 470 | N | MET | A | 65 | 4632 | 7891 | 5910 | −247 | 197 | −86 | N |
| ATOM | 471 | CA | MET | A | 65 | 74.978 | 43.276 | 17.958 | 1.00 | 48.13 | | C |
| ANISOU | 471 | CA | MET | A | 65 | 4622 | 7858 | 5808 | −74 | 124 | −26 | C |
| ATOM | 472 | CB | MET | A | 65 | 74.188 | 43.574 | 16.684 | 1.00 | 50.84 | | C |
| ANISOU | 472 | CB | MET | A | 65 | 4988 | 8093 | 6236 | −73 | 196 | 69 | C |
| ATOM | 473 | CG | MET | A | 65 | 72.756 | 43.926 | 16.938 | 1.00 | 55.04 | | C |
| ANISOU | 473 | CG | MET | A | 65 | 5630 | 8437 | 6846 | −121 | 254 | 117 | C |
| ATOM | 474 | SD | MET | A | 65 | 71.819 | 42.535 | 17.575 | 1.00 | 59.90 | | S |
| ANISOU | 474 | SD | MET | A | 65 | 6388 | 8975 | 7397 | −35 | 169 | 123 | S |
| ATOM | 475 | CE | MET | A | 65 | 70.397 | 43.421 | 18.253 | 1.00 | 56.83 | | C |
| ANISOU | 475 | CE | MET | A | 65 | 6063 | 8426 | 7103 | −143 | 265 | 142 | C |
| ATOM | 476 | C | MET | A | 65 | 76.277 | 42.601 | 17.599 | 1.00 | 51.25 | | C |
| ANISOU | 476 | C | MET | A | 65 | 4905 | 8472 | 6096 | 41 | 43 | −67 | C |
| ATOM | 477 | O | MET | A | 65 | 77.339 | 43.214 | 17.566 | 1.00 | 49.99 | | O |
| ANISOU | 477 | O | MET | A | 65 | 4590 | 8481 | 5925 | −33 | 65 | −132 | O |
| ATOM | 478 | N | ASP | A | 66 | 76.156 | 41.313 | 17.316 | 1.00 | 48.95 | | N |
| ANISOU | 478 | N | ASP | A | 66 | 4702 | 8181 | 5717 | 222 | −42 | −33 | N |
| ATOM | 479 | CA | ASP | A | 66 | 77.151 | 40.371 | 16.846 | 1.00 | 49.88 | | C |
| ANISOU | 479 | CA | ASP | A | 66 | 4770 | 8475 | 5710 | 404 | −125 | −49 | C |
| ATOM | 480 | CB | ASP | A | 66 | 76.336 | 39.167 | 16.349 | 1.00 | 53.34 | | C |
| ANISOU | 480 | CB | ASP | A | 66 | 5395 | 8769 | 6102 | 541 | −163 | 24 | C |
| ATOM | 481 | CG | ASP | A | 66 | 77.045 | 37.850 | 16.226 | 1.00 | 80.20 | | C |
| ANISOU | 481 | CG | ASP | A | 66 | 8868 | 12269 | 9337 | 775 | −260 | 14 | C |
| ATOM | 482 | OD1 | ASP | A | 66 | 78.129 | 37.811 | 15.592 | 1.00 | 84.83 | | O |
| ANISOU | 482 | OD1 | ASP | A | 66 | 9308 | 13058 | 9865 | 871 | −283 | −10 | O |
| ATOM | 483 | OD2 | ASP | A | 66 | 76.498 | 36.841 | 16.725 | 1.00 | 90.92 | | O |
| ANISOU | 483 | OD2 | ASP | A | 66 | 10440 | 13494 | 10612 | 866 | −302 | 30 | O |
| ATOM | 484 | C | ASP | A | 66 | 77.915 | 41.017 | 15.660 | 1.00 | 54.00 | | C |
| ANISOU | 484 | C | ASP | A | 66 | 5112 | 9132 | 6274 | 358 | −65 | −43 | C |
| ATOM | 485 | O | ASP | A | 66 | 77.288 | 41.673 | 14.815 | 1.00 | 53.32 | | O |
| ANISOU | 485 | O | ASP | A | 66 | 5034 | 8926 | 6299 | 259 | 27 | 20 | O |
| ATOM | 486 | N | ALA | A | 67 | 79.258 | 40.855 | 15.600 | 1.00 | 49.88 | | N |
| ANISOU | 486 | N | ALA | A | 67 | 4427 | 8871 | 5654 | 433 | −110 | −111 | N |
| ATOM | 487 | CA | ALA | A | 67 | 80.071 | 41.438 | 14.516 | 1.00 | 48.26 | | C |
| ANISOU | 487 | CA | ALA | A | 67 | 4042 | 8819 | 5474 | 377 | −41 | −116 | C |
| ATOM | 488 | CB | ALA | A | 67 | 81.524 | 41.053 | 14.666 | 1.00 | 48.95 | | C |
| ANISOU | 488 | CB | ALA | A | 67 | 3938 | 9236 | 5423 | 488 | −110 | −210 | C |
| ATOM | 489 | C | ALA | A | 67 | 79.563 | 41.066 | 13.134 | 1.00 | 49.30 | | C |
| ANISOU | 489 | C | ALA | A | 67 | 4248 | 8845 | 5640 | 457 | −7 | −12 | C |
| ATOM | 490 | O | ALA | A | 67 | 79.496 | 41.935 | 12.266 | 1.00 | 49.45 | | O |
| ANISOU | 490 | O | ALA | A | 67 | 4210 | 8834 | 5747 | 336 | 100 | 24 | O |
| ATOM | 491 | N | ASP | A | 68 | 79.143 | 39.802 | 12.942 | 1.00 | 43.53 | | N |
| ANISOU | 491 | N | ASP | A | 68 | 3669 | 8037 | 4831 | 654 | −91 | 34 | N |
| ATOM | 492 | CA | ASP | A | 68 | 78.576 | 39.353 | 11.668 | 1.00 | 42.16 | | C |
| ANISOU | 492 | CA | ASP | A | 68 | 3585 | 7760 | 4673 | 732 | −73 | 122 | C |
| ATOM | 493 | CB | ASP | A | 68 | 78.366 | 37.836 | 11.640 | 1.00 | 43.79 | | C |
| ANISOU | 493 | CB | ASP | A | 68 | 3971 | 7913 | 4753 | 946 | −173 | 140 | C |
| ATOM | 494 | CG | ASP | A | 68 | 79.655 | 37.041 | 11.600 | 1.00 | 57.84 | | C |
| ANISOU | 494 | CG | ASP | A | 68 | 5686 | 9919 | 6372 | 1159 | −247 | 91 | C |
| ATOM | 495 | OD1 | ASP | A | 68 | 80.454 | 37.249 | 10.664 | 1.00 | 58.46 | | O |
| ANISOU | 495 | OD1 | ASP | A | 68 | 5620 | 10162 | 6429 | 1210 | −220 | 93 | O |
| ATOM | 496 | OD2 | ASP | A | 68 | 79.858 | 36.199 | 12.495 | 1.00 | 68.35 | | O |
| ANISOU | 496 | OD2 | ASP | A | 68 | 7115 | 11271 | 7585 | 1291 | −329 | 52 | O |
| ATOM | 497 | C | ASP | A | 68 | 77.276 | 40.085 | 11.338 | 1.00 | 44.06 | | C |
| ANISOU | 497 | C | ASP | A | 68 | 3908 | 7782 | 5052 | 586 | 8 | 190 | C |
| ATOM | 498 | O | ASP | A | 68 | 77.071 | 40.429 | 10.188 | 1.00 | 44.82 | | O |
| ANISOU | 498 | O | ASP | A | 68 | 3987 | 7850 | 5192 | 578 | 66 | 249 | O |
| ATOM | 499 | N | THR | A | 69 | 76.431 | 40.362 | 12.347 | 1.00 | 38.42 | | N |
| ANISOU | 499 | N | THR | A | 69 | 3276 | 6928 | 4395 | 485 | 14 | 180 | N |
| ATOM | 500 | CA | THR | A | 69 | 75.162 | 41.092 | 12.216 | 1.00 | 36.92 | | C |
| ANISOU | 500 | CA | THR | A | 69 | 3153 | 6553 | 4320 | 368 | 89 | 234 | C |
| ATOM | 501 | CB | THR | A | 69 | 74.329 | 40.951 | 13.489 | 1.00 | 41.20 | | C |
| ANISOU | 501 | CB | THR | A | 69 | 3802 | 6975 | 4877 | 314 | 65 | 209 | C |
| ATOM | 502 | OG1 | THR | A | 69 | 74.297 | 39.580 | 13.857 | 1.00 | 41.75 | | O |
| ANISOU | 502 | OG1 | THR | A | 69 | 3990 | 7037 | 4834 | 442 | −31 | 194 | O |
| ATOM | 503 | CG2 | THR | A | 69 | 72.913 | 41.457 | 13.321 | 1.00 | 39.42 | | C |
| ANISOU | 503 | CG2 | THR | A | 69 | 3645 | 6587 | 4746 | 236 | 128 | 262 | C |
| ATOM | 504 | C | THR | A | 69 | 75.414 | 42.562 | 11.871 | 1.00 | 38.42 | | C |
| ANISOU | 504 | C | THR | A | 69 | 3243 | 6750 | 4605 | 224 | 210 | 245 | C |
| ATOM | 505 | O | THR | A | 69 | 74.668 | 43.124 | 11.069 | 1.00 | 37.28 | | O |
| ANISOU | 505 | O | THR | A | 69 | 3135 | 6505 | 4525 | 203 | 280 | 314 | O |
| ATOM | 506 | N | VAL | A | 70 | 76.446 | 43.193 | 12.478 | 1.00 | 33.67 | | N |
| ANISOU | 506 | N | VAL | A | 70 | 2528 | 6268 | 3997 | 125 | 239 | 171 | N |
| ATOM | 507 | CA | VAL | A | 70 | 76.779 | 44.577 | 12.142 | 1.00 | 32.53 | | C |
| ANISOU | 507 | CA | VAL | A | 70 | 2319 | 6118 | 3925 | −40 | 374 | 169 | C |
| ATOM | 508 | CB | VAL | A | 70 | 77.796 | 45.208 | 13.115 | 1.00 | 34.81 | | C |
| ANISOU | 508 | CB | VAL | A | 70 | 2496 | 6534 | 4197 | −191 | 396 | 56 | C |
| ATOM | 509 | CG1 | VAL | A | 70 | 78.414 | 46.472 | 12.525 | 1.00 | 34.04 | | C |
| ANISOU | 509 | CG1 | VAL | A | 70 | 2325 | 6467 | 4141 | −374 | 547 | 41 | C |
| ATOM | 510 | CG2 | VAL | A | 70 | 77.141 | 45.511 | 14.448 | 1.00 | 34.41 | | C |
| ANISOU | 510 | CG2 | VAL | A | 70 | 2537 | 6356 | 4183 | −271 | 387 | 22 | C |
| ATOM | 511 | C | VAL | A | 70 | 77.264 | 44.595 | 10.687 | 1.00 | 37.37 | | C |
| ANISOU | 511 | C | VAL | A | 70 | 2872 | 6806 | 4520 | 12 | 422 | 219 | C |
| ATOM | 512 | O | VAL | A | 70 | 76.699 | 45.332 | 9.877 | 1.00 | 37.15 | | O |
| ANISOU | 512 | O | VAL | A | 70 | 2906 | 6658 | 4552 | −24 | 523 | 294 | O |
| ATOM | 513 | N | ALA | A | 71 | 78.265 | 43.732 | 10.352 | 1.00 | 33.83 | | N |
| ANISOU | 513 | N | ALA | A | 71 | 2319 | 6559 | 3977 | 128 | 346 | 184 | N |
| ATOM | 514 | CA | ALA | A | 71 | 78.841 | 43.608 | 9.005 | 1.00 | 33.14 | | C |
| ANISOU | 514 | CA | ALA | A | 71 | 2165 | 6574 | 3852 | 200 | 383 | 223 | C |
| ATOM | 515 | CB | ALA | A | 71 | 79.911 | 42.523 | 8.990 | 1.00 | 33.84 | | C |
| ANISOU | 515 | CB | ALA | A | 71 | 2146 | 6897 | 3816 | 360 | 278 | 166 | C |
| ATOM | 516 | C | ALA | A | 71 | 77.755 | 43.328 | 7.957 | 1.00 | 35.34 | | C |
| ANISOU | 516 | C | ALA | A | 71 | 2580 | 6690 | 4157 | 304 | 388 | 334 | C |
| ATOM | 517 | O | ALA | A | 71 | 77.738 | 44.001 | 6.933 | 1.00 | 35.08 | | O |
| ANISOU | 517 | O | ALA | A | 71 | 2550 | 6626 | 4154 | 272 | 490 | 392 | O |
| ATOM | 518 | N | ALA | A | 72 | 76.804 | 42.399 | 8.265 | 1.00 | 31.40 | | N |
| ANISOU | 518 | N | ALA | A | 72 | 2204 | 6084 | 3643 | 410 | 287 | 356 | N |
| ATOM | 519 | CA | ALA | A | 72 | 75.634 | 42.037 | 7.450 | 1.00 | 31.04 | | C |
| ANISOU | 519 | CA | ALA | A | 72 | 2277 | 5907 | 3612 | 491 | 272 | 435 | C |
| ATOM | 520 | CB | ALA | A | 72 | 74.842 | 40.929 | 8.116 | 1.00 | 31.48 | | C |
| ANISOU | 520 | CB | ALA | A | 72 | 2446 | 5886 | 3629 | 558 | 163 | 418 | C |
| ATOM | 521 | C | ALA | A | 72 | 74.737 | 43.252 | 7.239 | 1.00 | 35.19 | | C |
| ANISOU | 521 | C | ALA | A | 72 | 2844 | 6292 | 4233 | 390 | 380 | 491 | C |
| ATOM | 522 | O | ALA | A | 72 | 74.072 | 43.352 | 6.216 | 1.00 | 35.10 | | O |
| ANISOU | 522 | O | ALA | A | 72 | 2880 | 6231 | 4226 | 454 | 407 | 562 | O |
| ATOM | 523 | N | GLY | A | 73 | 74.765 | 44.175 | 8.191 | 1.00 | 31.71 | | N |
| ANISOU | 523 | N | GLY | A | 73 | 2395 | 5800 | 3855 | 250 | 443 | 458 | N |
| ATOM | 524 | CA | GLY | A | 73 | 74.039 | 45.431 | 8.118 | 1.00 | 31.54 | | C |
| ANISOU | 524 | CA | GLY | A | 73 | 2437 | 5638 | 3910 | 166 | 563 | 506 | C |
| ATOM | 525 | C | GLY | A | 73 | 74.649 | 46.334 | 7.070 | 1.00 | 35.81 | | C |
| ANISOU | 525 | C | GLY | A | 73 | 2964 | 6191 | 4452 | 130 | 692 | 551 | C |
| ATOM | 526 | O | GLY | A | 73 | 73.920 | 47.024 | 6.371 | 1.00 | 35.84 | | O |
| ANISOU | 526 | O | GLY | A | 73 | 3057 | 6084 | 4476 | 167 | 771 | 631 | O |
| ATOM | 527 | N | LEU | A | 74 | 75.977 | 46.321 | 6.913 | 1.00 | 32.88 | | N |
| ANISOU | 527 | N | LEU | A | 74 | 2483 | 5966 | 4044 | 69 | 718 | 501 | N |
| ATOM | 528 | CA | LEU | A | 74 | 76.610 | 47.146 | 5.869 | 1.00 | 32.85 | | C |
| ANISOU | 528 | CA | LEU | A | 74 | 2471 | 5979 | 4030 | 13 | 860 | 540 | C |
| ATOM | 529 | CB | LEU | A | 74 | 78.115 | 47.391 | 6.149 | 1.00 | 32.24 | | C |
| ANISOU | 529 | CB | LEU | A | 74 | 2241 | 6086 | 3923 | −133 | 909 | 445 | C |
| ATOM | 530 | CG | LEU | A | 74 | 78.488 | 48.095 | 7.464 | 1.00 | 34.13 | | C |
| ANISOU | 530 | CG | LEU | A | 74 | 2444 | 6327 | 4198 | −340 | 952 | 345 | C |
| ATOM | 531 | CD1 | LEU | A | 74 | 79.619 | 47.370 | 8.145 | 1.00 | 33.00 | | C |
| ANISOU | 531 | CD1 | LEU | A | 74 | 2107 | 6440 | 3992 | −356 | 851 | 226 | C |
| ATOM | 532 | CD2 | LEU | A | 74 | 78.841 | 49.540 | 7.225 | 1.00 | 33.28 | | C |
| ANISOU | 532 | CD2 | LEU | A | 74 | 2398 | 6128 | 4119 | −555 | 1158 | 344 | C |
| ATOM | 533 | C | LEU | A | 74 | 76.417 | 46.523 | 4.468 | 1.00 | 39.03 | | C |
| ANISOU | 533 | C | LEU | A | 74 | 3275 | 6793 | 4761 | 191 | 829 | 621 | C |
| ATOM | 534 | O | LEU | A | 74 | 76.380 | 47.236 | 3.454 | 1.00 | 40.11 | | O |
| ANISOU | 534 | O | LEU | A | 74 | 3479 | 6870 | 4892 | 198 | 946 | 695 | O |
| ATOM | 535 | N | LEU | A | 75 | 76.249 | 45.198 | 4.415 | 1.00 | 34.74 | | N |
| ANISOU | 535 | N | LEU | A | 75 | 2702 | 6330 | 4170 | 338 | 677 | 608 | N |
| ATOM | 536 | CA | LEU | A | 75 | 76.138 | 44.505 | 3.132 | 1.00 | 33.19 | | C |
| ANISOU | 536 | CA | LEU | A | 75 | 2526 | 6175 | 3908 | 502 | 634 | 665 | C |
| ATOM | 537 | CB | LEU | A | 75 | 77.039 | 43.261 | 3.162 | 1.00 | 32.25 | | C |
| ANISOU | 537 | CB | LEU | A | 75 | 2320 | 6227 | 3705 | 600 | 523 | 605 | C |
| ATOM | 538 | CG | LEU | A | 75 | 78.460 | 43.498 | 3.651 | 1.00 | 34.96 | | C |
| ANISOU | 538 | CG | LEU | A | 75 | 2508 | 6750 | 4024 | 512 | 565 | 527 | C |
| ATOM | 539 | CD1 | LEU | A | 75 | 78.969 | 42.317 | 4.376 | 1.00 | 34.67 | | C |
| ANISOU | 539 | CD1 | LEU | A | 75 | 2415 | 6846 | 3913 | 617 | 428 | 453 | C |
| ATOM | 540 | CD2 | LEU | A | 75 | 79.383 | 43.936 | 2.531 | 1.00 | 33.91 | | C |
| ANISOU | 540 | CD2 | LEU | A | 75 | 2304 | 6729 | 3852 | 508 | 674 | 554 | C |
| ATOM | 541 | C | LEU | A | 75 | 74.734 | 44.121 | 2.705 | 1.00 | 37.32 | | C |
| ANISOU | 541 | C | LEU | A | 75 | 3159 | 6588 | 4433 | 616 | 568 | 724 | C |
| ATOM | 542 | O | LEU | A | 75 | 74.583 | 43.721 | 1.572 | 1.00 | 36.67 | | O |
| ANISOU | 542 | O | LEU | A | 75 | 3105 | 6536 | 4294 | 737 | 545 | 771 | O |
| ATOM | 543 | N | HIS | A | 76 | 73.716 | 44.251 | 3.570 | 1.00 | 36.06 | | N |
| ANISOU | 543 | N | HIS | A | 76 | 3052 | 6321 | 4327 | 576 | 539 | 715 | N |
| ATOM | 544 | CA | HIS | A | 76 | 72.327 | 43.836 | 3.316 | 1.00 | 37.00 | | C |
| ANISOU | 544 | CA | HIS | A | 76 | 3241 | 6377 | 4440 | 662 | 470 | 747 | C |
| ATOM | 545 | CB | HIS | A | 76 | 71.419 | 44.232 | 4.486 | 1.00 | 37.67 | | C |
| ANISOU | 545 | CB | HIS | A | 76 | 3356 | 6367 | 4592 | 582 | 471 | 723 | C |
| ATOM | 546 | CG | HIS | A | 76 | 70.952 | 45.649 | 4.448 | 1.00 | 41.17 | | C |
| ANISOU | 546 | CG | HIS | A | 76 | 3848 | 6710 | 5086 | 552 | 598 | 777 | C |
| ATOM | 547 | ND1 | HIS | A | 76 | 71.755 | 46.673 | 4.875 | 1.00 | 42.92 | | N |
| ANISOU | 547 | ND1 | HIS | A | 76 | 4078 | 6885 | 5347 | 433 | 718 | 769 | N |
| ATOM | 548 | CE1 | HIS | A | 76 | 71.046 | 47.778 | 4.718 | 1.00 | 42.64 | | C |
| ANISOU | 548 | CE1 | HIS | A | 76 | 4134 | 6733 | 5333 | 450 | 822 | 828 | C |
| ATOM | 549 | NE2 | HIS | A | 76 | 69.846 | 47.521 | 4.201 | 1.00 | 43.31 | | N |
| ANISOU | 549 | NE2 | HIS | A | 76 | 4244 | 6820 | 5394 | 591 | 766 | 873 | N |
| ATOM | 550 | CD2 | HIS | A | 76 | 69.775 | 46.163 | 4.017 | 1.00 | 43.50 | | C |
| ANISOU | 550 | CD2 | HIS | A | 76 | 4197 | 6949 | 5381 | 636 | 624 | 835 | C |
| ATOM | 551 | C | HIS | A | 76 | 71.708 | 44.304 | 1.967 | 1.00 | 43.80 | | C |
| ANISOU | 551 | C | HIS | A | 76 | 4150 | 7219 | 5271 | 769 | 517 | 833 | C |
| ATOM | 552 | O | HIS | A | 76 | 70.694 | 43.732 | 1.529 | 1.00 | 42.01 | | O |
| ANISOU | 552 | O | HIS | A | 76 | 3949 | 7001 | 5010 | 857 | 437 | 844 | O |
| ATOM | 553 | N | ASP | A | 77 | 72.313 | 45.316 | 1.310 | 1.00 | 43.75 | | N |
| ANISOU | 553 | N | ASP | A | 77 | 4162 | 7194 | 5265 | 757 | 649 | 889 | N |
| ATOM | 554 | CA | ASP | A | 77 | 71.793 | 45.777 | 0.025 | 1.00 | 44.91 | | C |
| ANISOU | 554 | CA | ASP | A | 77 | 4379 | 7321 | 5363 | 883 | 701 | 978 | C |
| ATOM | 555 | CB | ASP | A | 77 | 71.271 | 47.214 | 0.130 | 1.00 | 47.75 | | C |
| ANISOU | 555 | CB | ASP | A | 77 | 4837 | 7548 | 5757 | 865 | 841 | 1039 | C |
| ATOM | 556 | CG | ASP | A | 77 | 69.826 | 47.301 | 0.608 | 1.00 | 67.20 | | C |
| ANISOU | 556 | CG | ASP | A | 77 | 7326 | 9966 | 8241 | 926 | 787 | 1042 | C |
| ATOM | 557 | OD1 | ASP | A | 77 | 68.966 | 46.567 | 0.055 | 1.00 | 70.11 | | O |
| ANISOU | 557 | OD1 | ASP | A | 77 | 7666 | 10412 | 8560 | 1047 | 676 | 1045 | O |
| ATOM | 558 | OD2 | ASP | A | 77 | 69.547 | 48.127 | 1.507 | 1.00 | 74.86 | | O |
| ANISOU | 558 | OD2 | ASP | A | 77 | 8343 | 10834 | 9268 | 851 | 861 | 1036 | O |
| ATOM | 559 | C | ASP | A | 77 | 72.787 | 45.596 | −1.144 | 1.00 | 48.84 | | C |
| ANISOU | 559 | C | ASP | A | 77 | 4865 | 7900 | 5793 | 937 | 743 | 1010 | C |
| ATOM | 560 | O | ASP | A | 77 | 72.488 | 45.997 | −2.273 | 1.00 | 47.71 | | O |
| ANISOU | 560 | O | ASP | A | 77 | 4792 | 7740 | 5595 | 1045 | 800 | 1088 | O |
| ATOM | 561 | N | THR | A | 78 | 73.927 | 44.931 | −0.888 | 1.00 | 46.16 | | N |
| ANISOU | 561 | N | THR | A | 78 | 4435 | 7663 | 5441 | 885 | 711 | 948 | N |
| ATOM | 562 | CA | THR | A | 78 | 74.939 | 44.700 | −1.910 | 1.00 | 46.95 | | C |
| ANISOU | 562 | CA | THR | A | 78 | 4502 | 7866 | 5472 | 935 | 754 | 967 | C |
| ATOM | 563 | CB | THR | A | 78 | 76.295 | 44.323 | −1.322 | 1.00 | 46.94 | | C |
| ANISOU | 563 | CB | THR | A | 78 | 4374 | 7994 | 5466 | 849 | 755 | 887 | C |
| ATOM | 564 | OG1 | THR | A | 78 | 76.168 | 43.124 | −0.562 | 1.00 | 41.92 | | O |
| ANISOU | 564 | OG1 | THR | A | 78 | 3704 | 7407 | 4818 | 894 | 599 | 816 | O |
| ATOM | 565 | CG2 | THR | A | 78 | 76.955 | 45.457 | −0.548 | 1.00 | 45.08 | | C |
| ANISOU | 565 | CG2 | THR | A | 78 | 4096 | 7737 | 5296 | 648 | 892 | 856 | C |
| ATOM | 566 | C | THR | A | 78 | 74.546 | 43.688 | −2.969 | 1.00 | 57.08 | | C |
| ANISOU | 566 | C | THR | A | 78 | 5817 | 9205 | 6666 | 1110 | 652 | 991 | C |
| ATOM | 567 | O | THR | A | 78 | 75.134 | 43.705 | −4.058 | 1.00 | 58.67 | | O |
| ANISOU | 567 | O | THR | A | 78 | 6024 | 9467 | 6800 | 1182 | 708 | 1032 | O |
| ATOM | 568 | N | LEU | A | 79 | 73.621 | 42.793 | −2.649 | 1.00 | 56.48 | | N |
| ANISOU | 568 | N | LEU | A | 79 | 5767 | 9113 | 6581 | 1166 | 513 | 960 | N |
| ATOM | 569 | CA | LEU | A | 79 | 73.224 | 41.732 | −3.608 | 1.00 | 58.21 | | C |
| ANISOU | 569 | CA | LEU | A | 79 | 6027 | 9385 | 6705 | 1306 | 415 | 964 | C |
| ATOM | 570 | CB | LEU | A | 79 | 72.383 | 40.695 | −2.867 | 1.00 | 58.04 | | C |
| ANISOU | 570 | CB | LEU | A | 79 | 6031 | 9350 | 6671 | 1302 | 269 | 892 | C |
| ATOM | 571 | CG | LEU | A | 79 | 73.174 | 39.753 | −1.970 | 1.00 | 62.45 | | C |
| ANISOU | 571 | CG | LEU | A | 79 | 6570 | 9932 | 7227 | 1260 | 212 | 816 | C |
| ATOM | 572 | CD1 | LEU | A | 79 | 72.247 | 38.788 | −1.262 | 1.00 | 62.65 | | C |
| ANISOU | 572 | CD1 | LEU | A | 79 | 6664 | 9904 | 7237 | 1234 | 99 | 754 | C |
| ATOM | 573 | CD2 | LEU | A | 79 | 74.202 | 38.992 | −2.774 | 1.00 | 64.75 | | C |
| ANISOU | 573 | CD2 | LEU | A | 79 | 6854 | 10323 | 7425 | 1367 | 202 | 807 | C |
| ATOM | 574 | C | LEU | A | 79 | 72.478 | 42.247 | −4.845 | 1.00 | 67.45 | | C |
| ANISOU | 574 | C | LEU | A | 79 | 7263 | 10542 | 7823 | 1418 | 453 | 1046 | C |
| ATOM | 575 | O | LEU | A | 79 | 72.743 | 41.735 | −5.935 | 1.00 | 68.29 | | O |
| ANISOU | 575 | O | LEU | A | 79 | 7392 | 10707 | 7848 | 1517 | 475 | 1082 | O |
| ATOM | 576 | N | GLU | A | 80 | 71.550 | 43.198 | −4.707 | 1.00 | 66.61 | | N |
| ANISOU | 576 | N | GLU | A | 80 | 7186 | 10371 | 7751 | 1419 | 464 | 1076 | N |
| ATOM | 577 | C | GLU | A | 80 | 71.149 | 44.641 | −6.806 | 1.00 | 77.08 | | C |
| ANISOU | 577 | C | GLU | A | 80 | 8644 | 11669 | 8974 | 1613 | 642 | 1245 | C |
| ATOM | 578 | O | GLU | A | 80 | 71.280 | 44.383 | −8.016 | 1.00 | 78.18 | | O |
| ANISOU | 578 | O | GLU | A | 80 | 8821 | 11866 | 9019 | 1726 | 650 | 1284 | O |
| ATOM | 579 | CA | GLU | A | 80 | 70.677 | 43.517 | −5.874 | 1.00 | 68.68 | | C |
| ANISOU | 579 | CA | GLU | A | 80 | 7508 | 10640 | 7947 | 1557 | 482 | 1147 | C |
| ATOM | 580 | CB | GLU | A | 80 | 69.300 | 43.918 | −5.345 | 1.00 | 70.65 | | C |
| ANISOU | 580 | CB | GLU | A | 80 | 7757 | 10848 | 8238 | 1556 | 464 | 1148 | C |
| ATOM | 581 | CG | GLU | A | 80 | 68.147 | 43.292 | −6.104 | 1.00 | 87.31 | | C |
| ANISOU | 581 | CG | GLU | A | 80 | 9875 | 13044 | 10256 | 1709 | 382 | 1163 | C |
| ATOM | 582 | CD | GLU | A | 80 | 66.798 | 43.506 | −5.443 | 1.00 | 121.25 | | C |
| ANISOU | 582 | CD | GLU | A | 80 | 14102 | 17388 | 14580 | 1667 | 278 | 1091 | C |
| ATOM | 583 | OE1 | GLU | A | 80 | 66.549 | 44.630 | −4.971 | 1.00 | 118.56 | | O |
| ANISOU | 583 | OE1 | GLU | A | 80 | 13720 | 17138 | 14189 | 1647 | 150 | 1012 | O |
| ATOM | 584 | OE2 | GLU | A | 80 | 66.006 | 42.547 | −5.398 | 1.00 | 122.37 | | O |
| ANISOU | 584 | OE2 | GLU | A | 80 | 14238 | 17471 | 14785 | 1641 | 334 | 1106 | O |
| ATOM | 585 | N | ASP | A | 81 | 71.415 | 45.827 | −6.266 | 1.00 | 74.65 | | N |
| ANISOU | 585 | N | ASP | A | 81 | 8379 | 11253 | 8733 | 1534 | 774 | 1283 | N |
| ATOM | 586 | CA | ASP | A | 81 | 71.733 | 47.038 | −7.065 | 1.00 | 74.88 | | C |
| ANISOU | 586 | CA | ASP | A | 81 | 8524 | 11188 | 8738 | 1545 | 965 | 1376 | C |
| ATOM | 587 | CB | ASP | A | 81 | 71.632 | 48.269 | −6.159 | 1.00 | 77.96 | | C |
| ANISOU | 587 | CB | ASP | A | 81 | 8949 | 11445 | 9228 | 1381 | 1086 | 1367 | C |
| ATOM | 588 | CG | ASP | A | 81 | 70.861 | 47.998 | −4.877 | 1.00 | 93.65 | | C |
| ANISOU | 588 | CG | ASP | A | 81 | 10918 | 13387 | 11276 | 1379 | 1007 | 1333 | C |
| ATOM | 589 | OD2 | ASP | A | 81 | 70.341 | 48.956 | −4.284 | 1.00 | 99.98 | | O |
| ANISOU | 589 | OD2 | ASP | A | 81 | 11839 | 14069 | 12079 | 1418 | 1110 | 1390 | O |
| ATOM | 590 | OD1 | ASP | A | 81 | 70.778 | 46.825 | −4.493 | 1.00 | 95.20 | | O |
| ANISOU | 590 | OD1 | ASP | A | 81 | 10999 | 13663 | 11510 | 1339 | 856 | 1250 | O |
| ATOM | 591 | C | ASP | A | 81 | 73.087 | 46.978 | −7.778 | 1.00 | 73.85 | | C |
| ANISOU | 591 | C | ASP | A | 81 | 8404 | 11104 | 8552 | 1528 | 1067 | 1404 | C |
| ATOM | 592 | O | ASP | A | 81 | 73.178 | 47.472 | −8.904 | 1.00 | 70.59 | | O |
| ANISOU | 592 | O | ASP | A | 81 | 8108 | 10663 | 8051 | 1643 | 1163 | 1493 | O |
| ATOM | 593 | O | CYS | A | 82 | 76.876 | 45.703 | −9.230 | 1.00 | 72.49 | | O |
| ANISOU | 593 | O | CYS | A | 82 | 8036 | 11271 | 8237 | 1457 | 1209 | 1353 | O |
| ATOM | 594 | N | CYS | A | 82 | 74.080 | 46.384 | −7.129 | 1.00 | 69.85 | | N |
| ANISOU | 594 | N | CYS | A | 82 | 7773 | 10682 | 8083 | 1402 | 1044 | 1325 | N |
| ATOM | 595 | CA | CYS | A | 82 | 75.486 | 46.441 | −7.511 | 1.00 | 68.80 | | C |
| ANISOU | 595 | CA | CYS | A | 82 | 7598 | 10620 | 7922 | 1329 | 1159 | 1322 | C |
| ATOM | 596 | C | CYS | A | 82 | 76.009 | 45.383 | −8.426 | 1.00 | 71.64 | | C |
| ANISOU | 596 | C | CYS | A | 82 | 7907 | 11122 | 8190 | 1452 | 1083 | 1312 | C |
| ATOM | 597 | CB | CYS | A | 82 | 76.347 | 46.483 | −6.254 | 1.00 | 68.19 | | C |
| ANISOU | 597 | CB | CYS | A | 82 | 7396 | 10580 | 7932 | 1121 | 1189 | 1232 | C |
| ATOM | 598 | SG | CYS | A | 82 | 75.714 | 47.573 | −4.956 | 1.00 | 71.87 | | S |
| ANISOU | 598 | SG | CYS | A | 82 | 7882 | 10912 | 8513 | 1002 | 1163 | 1196 | S |
| ATOM | 599 | N | GLY | A | 83 | 75.670 | 44.131 | −8.196 | 1.00 | 66.29 | | N |
| ANISOU | 599 | N | GLY | A | 83 | 7163 | 10522 | 7503 | 1518 | 902 | 1244 | N |
| ATOM | 600 | CA | GLY | A | 83 | 76.291 | 43.073 | −8.977 | 1.00 | 64.91 | | C |
| ANISOU | 600 | CA | GLY | A | 83 | 6957 | 10474 | 7232 | 1633 | 841 | 1224 | C |
| ATOM | 601 | C | GLY | A | 83 | 77.353 | 42.366 | −8.163 | 1.00 | 64.86 | | C |
| ANISOU | 601 | C | GLY | A | 83 | 6819 | 10580 | 7244 | 1570 | 793 | 1131 | C |
| ATOM | 602 | O | GLY | A | 83 | 78.540 | 42.332 | −8.529 | 1.00 | 61.72 | | O |
| ANISOU | 602 | O | GLY | A | 83 | 6346 | 10308 | 6796 | 1580 | 861 | 1117 | O |
| ATOM | 603 | N | VAL | A | 84 | 76.890 | 41.857 | −6.995 | 1.00 | 60.40 | | N |
| ANISOU | 603 | N | VAL | A | 84 | 6225 | 9978 | 6747 | 1510 | 682 | 1064 | N |
| ATOM | 604 | CA | VAL | A | 84 | 77.616 | 41.028 | −6.035 | 1.00 | 59.33 | | C |
| ANISOU | 604 | CA | VAL | A | 84 | 5997 | 9928 | 6618 | 1484 | 600 | 972 | C |
| ATOM | 605 | CB | VAL | A | 84 | 77.815 | 41.702 | −4.639 | 1.00 | 61.98 | | C |
| ANISOU | 605 | CB | VAL | A | 84 | 6251 | 10237 | 7061 | 1303 | 638 | 925 | C |
| ATOM | 606 | CG1 | VAL | A | 84 | 78.170 | 40.683 | −3.561 | 1.00 | 61.54 | | C |
| ANISOU | 606 | CG1 | VAL | A | 84 | 6144 | 10241 | 6998 | 1316 | 511 | 833 | C |
| ATOM | 607 | CG2 | VAL | A | 84 | 78.878 | 42.794 | −4.703 | 1.00 | 61.52 | | C |
| ANISOU | 607 | CG2 | VAL | A | 84 | 6095 | 10253 | 7025 | 1169 | 809 | 928 | C |
| ATOM | 608 | C | VAL | A | 84 | 76.846 | 39.697 | −5.970 | 1.00 | 62.24 | | C |
| ANISOU | 608 | C | VAL | A | 84 | 6452 | 10261 | 6933 | 1595 | 429 | 935 | C |
| ATOM | 609 | O | VAL | A | 84 | 75.621 | 39.698 | −5.825 | 1.00 | 61.41 | | O |
| ANISOU | 609 | O | VAL | A | 84 | 6420 | 10052 | 6861 | 1576 | 371 | 944 | O |
| ATOM | 610 | N | ALA | A | 85 | 77.557 | 38.579 | −6.155 | 1.00 | 58.44 | | N |
| ANISOU | 610 | N | ALA | A | 85 | 5974 | 9875 | 6356 | 1715 | 361 | 891 | N |
| ATOM | 611 | CA | ALA | A | 85 | 76.969 | 37.251 | −6.099 | 1.00 | 58.15 | | C |
| ANISOU | 611 | CA | ALA | A | 85 | 6058 | 9795 | 6243 | 1813 | 220 | 845 | C |
| ATOM | 612 | CB | ALA | A | 85 | 77.846 | 36.264 | −6.862 | 1.00 | 58.82 | | C |
| ANISOU | 612 | CB | ALA | A | 85 | 6171 | 9987 | 6192 | 1986 | 199 | 828 | C |
| ATOM | 613 | C | ALA | A | 85 | 76.876 | 36.849 | −4.632 | 1.00 | 63.10 | | C |
| ANISOU | 613 | C | ALA | A | 85 | 6681 | 10378 | 6917 | 1739 | 151 | 778 | C |
| ATOM | 614 | O | ALA | A | 85 | 77.800 | 37.160 | −3.874 | 1.00 | 64.34 | | O |
| ANISOU | 614 | O | ALA | A | 85 | 6722 | 10619 | 7106 | 1695 | 192 | 750 | O |
| ATOM | 615 | N | PRO | A | 86 | 75.818 | 36.140 | −4.182 | 1.00 | 58.49 | | N |
| ANISOU | 615 | N | PRO | A | 86 | 6216 | 9679 | 6328 | 1713 | 53 | 742 | N |
| ATOM | 616 | CA | PRO | A | 86 | 75.779 | 35.721 | −2.763 | 1.00 | 57.64 | | C |
| ANISOU | 616 | CA | PRO | A | 86 | 6128 | 9523 | 6250 | 1650 | −1 | 682 | C |
| ATOM | 617 | CB | PRO | A | 86 | 74.456 | 34.958 | −2.647 | 1.00 | 59.66 | | C |
| ANISOU | 617 | CB | PRO | A | 86 | 6535 | 9649 | 6486 | 1605 | −85 | 651 | C |
| ATOM | 618 | CG | PRO | A | 86 | 74.079 | 34.601 | −4.041 | 1.00 | 64.46 | | C |
| ANISOU | 618 | CG | PRO | A | 86 | 7218 | 10269 | 7005 | 1693 | −105 | 671 | C |
| ATOM | 619 | CD | PRO | A | 86 | 74.628 | 35.674 | −4.920 | 1.00 | 59.99 | | C |
| ANISOU | 619 | CD | PRO | A | 86 | 6528 | 9793 | 6472 | 1730 | −10 | 745 | C |
| ATOM | 620 | C | PRO | A | 86 | 76.980 | 34.852 | −2.383 | 1.00 | 59.29 | | C |
| ANISOU | 620 | C | PRO | A | 86 | 6347 | 9823 | 6357 | 1782 | −35 | 635 | C |
| ATOM | 621 | O | PRO | A | 86 | 77.342 | 34.750 | −1.214 | 1.00 | 58.92 | | O |
| ANISOU | 621 | O | PRO | A | 86 | 6275 | 9788 | 6325 | 1757 | −58 | 592 | O |
| ATOM | 622 | N | GLU | A | 87 | 77.614 | 34.265 | −3.400 | 1.00 | 54.97 | | N |
| ANISOU | 622 | N | GLU | A | 87 | 5836 | 9355 | 5697 | 1943 | −35 | 645 | N |
| ATOM | 623 | CA | GLU | A | 87 | 78.796 | 33.423 | −3.300 | 1.00 | 54.63 | | C |
| ANISOU | 623 | CA | GLU | A | 87 | 5802 | 9427 | 5528 | 2123 | −61 | 607 | C |
| ATOM | 624 | CB | GLU | A | 87 | 79.030 | 32.688 | −4.629 | 1.00 | 56.28 | | C |
| ANISOU | 624 | CB | GLU | A | 87 | 6117 | 9665 | 5602 | 2300 | −70 | 623 | C |
| ATOM | 625 | CG | GLU | A | 87 | 77.933 | 31.669 | −4.953 | 1.00 | 68.53 | | C |
| ANISOU | 625 | CG | GLU | A | 87 | 7921 | 11036 | 7084 | 2311 | −149 | 604 | C |
| ATOM | 626 | CD | GLU | A | 87 | 76.901 | 32.034 | −6.011 | 1.00 | 87.96 | | C |
| ANISOU | 626 | CD | GLU | A | 87 | 10407 | 13435 | 9578 | 2228 | −140 | 641 | C |
| ATOM | 627 | OE1 | GLU | A | 87 | 76.443 | 33.200 | −6.035 | 1.00 | 75.83 | | O |
| ANISOU | 627 | OE1 | GLU | A | 87 | 8732 | 11913 | 8169 | 2098 | −86 | 685 | O |
| ATOM | 628 | OE2 | GLU | A | 87 | 76.534 | 31.140 | −6.809 | 1.00 | 81.52 | | O |
| ANISOU | 628 | OE2 | GLU | A | 87 | 9765 | 12559 | 8648 | 2303 | −187 | 621 | O |
| ATOM | 629 | C | GLU | A | 87 | 80.022 | 34.229 | −2.851 | 1.00 | 56.44 | | C |
| ANISOU | 629 | C | GLU | A | 87 | 5794 | 9853 | 5795 | 2105 | 8 | 596 | C |
| ATOM | 630 | O | GLU | A | 87 | 80.809 | 33.720 | −2.042 | 1.00 | 56.93 | | O |
| ANISOU | 630 | O | GLU | A | 87 | 5826 | 10016 | 5789 | 2204 | −33 | 543 | O |
| ATOM | 631 | N | GLU | A | 88 | 80.141 | 35.504 | −3.328 | 1.00 | 49.46 | | N |
| ANISOU | 631 | N | GLU | A | 88 | 4756 | 9023 | 5012 | 1971 | 118 | 640 | N |
| ATOM | 632 | CA | GLU | A | 88 | 81.208 | 36.452 | −2.976 | 1.00 | 47.30 | | C |
| ANISOU | 632 | CA | GLU | A | 88 | 4256 | 8929 | 4786 | 1883 | 212 | 620 | C |
| ATOM | 633 | CB | GLU | A | 88 | 81.157 | 37.736 | −3.838 | 1.00 | 48.45 | | C |
| ANISOU | 633 | CB | GLU | A | 88 | 4325 | 9074 | 5011 | 1748 | 354 | 685 | C |
| ATOM | 634 | CG | GLU | A | 88 | 82.441 | 38.556 | −3.768 | 1.00 | 59.34 | | C |
| ANISOU | 634 | CG | GLU | A | 88 | 5483 | 10668 | 6395 | 1663 | 474 | 654 | C |
| ATOM | 635 | CD | GLU | A | 88 | 82.643 | 39.752 | −4.686 | 1.00 | 78.83 | | C |
| ANISOU | 635 | CD | GLU | A | 88 | 7900 | 13140 | 8911 | 1528 | 646 | 713 | C |
| ATOM | 636 | OE1 | GLU | A | 88 | 81.957 | 39.845 | −5.731 | 1.00 | 70.62 | | O |
| ANISOU | 636 | OE1 | GLU | A | 88 | 6993 | 11982 | 7859 | 1583 | 673 | 794 | O |
| ATOM | 637 | OE2 | GLU | A | 88 | 83.520 | 40.589 | −4.362 | 1.00 | 70.69 | | O |
| ANISOU | 637 | OE2 | GLU | A | 88 | 6704 | 12239 | 7915 | 1365 | 759 | 673 | O |
| ATOM | 638 | C | GLU | A | 88 | 81.153 | 36.786 | −1.483 | 1.00 | 47.75 | | C |
| ANISOU | 638 | C | GLU | A | 88 | 4250 | 8971 | 4921 | 1752 | 185 | 567 | C |
| ATOM | 639 | O | GLU | A | 88 | 82.199 | 36.824 | −0.843 | 1.00 | 46.78 | | O |
| ANISOU | 639 | O | GLU | A | 88 | 3973 | 9036 | 4765 | 1765 | 190 | 505 | O |
| ATOM | 640 | N | LEU | A | 89 | 79.938 | 36.969 | −0.918 | 1.00 | 42.60 | | N |
| ANISOU | 640 | N | LEU | A | 89 | 3711 | 8116 | 4360 | 1636 | 152 | 583 | N |
| ATOM | 641 | CA | LEU | A | 89 | 79.744 | 37.268 | 0.511 | 1.00 | 41.00 | | C |
| ANISOU | 641 | CA | LEU | A | 89 | 3479 | 7869 | 4229 | 1513 | 125 | 536 | C |
| ATOM | 642 | CB | LEU | A | 89 | 78.310 | 37.713 | 0.828 | 1.00 | 39.54 | | C |
| ANISOU | 642 | CB | LEU | A | 89 | 3400 | 7469 | 4153 | 1375 | 125 | 572 | C |
| ATOM | 643 | CG | LEU | A | 89 | 77.845 | 39.012 | 0.158 | 1.00 | 43.32 | | C |
| ANISOU | 643 | CG | LEU | A | 89 | 3831 | 7897 | 4732 | 1245 | 244 | 636 | C |
| ATOM | 644 | CD1 | LEU | A | 89 | 76.364 | 39.204 | 0.296 | 1.00 | 43.09 | | C |
| ANISOU | 644 | CD1 | LEU | A | 89 | 3911 | 7689 | 4772 | 1180 | 226 | 672 | C |
| ATOM | 645 | CD2 | LEU | A | 89 | 78.554 | 40.209 | 0.712 | 1.00 | 45.13 | | C |
| ANISOU | 645 | CD2 | LEU | A | 89 | 3915 | 8197 | 5033 | 1087 | 345 | 610 | C |
| ATOM | 646 | C | LEU | A | 89 | 80.181 | 36.113 | 1.407 | 1.00 | 49.52 | | C |
| ANISOU | 646 | C | LEU | A | 89 | 4618 | 8998 | 5201 | 1657 | 15 | 472 | C |
| ATOM | 647 | O | LEU | A | 89 | 80.712 | 36.359 | 2.488 | 1.00 | 50.25 | | O |
| ANISOU | 647 | O | LEU | A | 89 | 4611 | 9177 | 5303 | 1611 | 2 | 415 | O |
| ATOM | 648 | N | GLU | A | 90 | 80.009 | 34.859 | 0.952 | 1.00 | 48.67 | | N |
| ANISOU | 648 | N | GLU | A | 90 | 4682 | 8838 | 4971 | 1842 | −59 | 477 | N |
| ATOM | 649 | CA | GLU | A | 90 | 80.441 | 33.680 | 1.717 | 1.00 | 49.45 | | C |
| ANISOU | 649 | CA | GLU | A | 90 | 4894 | 8960 | 4934 | 2020 | −152 | 425 | C |
| ATOM | 650 | CB | GLU | A | 90 | 79.767 | 32.403 | 1.182 | 1.00 | 51.52 | | C |
| ANISOU | 650 | CB | GLU | A | 90 | 5437 | 9050 | 5089 | 2147 | −214 | 439 | C |
| ATOM | 651 | CG | GLU | A | 90 | 79.818 | 31.216 | 2.140 | 1.00 | 65.58 | | C |
| ANISOU | 651 | CG | GLU | A | 90 | 7421 | 10758 | 6738 | 2289 | −294 | 394 | C |
| ATOM | 652 | CD | GLU | A | 90 | 78.890 | 30.068 | 1.781 | 1.00 | 95.13 | | C |
| ANISOU | 652 | CD | GLU | A | 90 | 11485 | 14274 | 10386 | 2337 | −334 | 397 | C |
| ATOM | 653 | OE1 | GLU | A | 90 | 78.864 | 29.669 | 0.594 | 1.00 | 102.58 | | O |
| ANISOU | 653 | OE1 | GLU | A | 90 | 12509 | 15214 | 11251 | 2441 | −329 | 415 | O |
| ATOM | 654 | OE2 | GLU | A | 90 | 78.195 | 29.560 | 2.690 | 1.00 | 87.98 | | O |
| ANISOU | 654 | OE2 | GLU | A | 90 | 10757 | 13194 | 9476 | 2257 | −363 | 375 | O |
| ATOM | 655 | C | GLU | A | 90 | 81.961 | 33.554 | 1.643 | 1.00 | 51.63 | | C |
| ANISOU | 655 | C | GLU | A | 90 | 5001 | 9516 | 5101 | 2191 | −148 | 384 | C |
| ATOM | 656 | O | GLU | A | 90 | 82.598 | 33.199 | 2.642 | 1.00 | 51.27 | | O |
| ANISOU | 656 | O | GLU | A | 90 | 4922 | 9580 | 4979 | 2288 | −202 | 327 | O |
| ATOM | 657 | N | ARG | A | 91 | 82.517 | 33.842 | 0.439 | 1.00 | 46.06 | | N |
| ANISOU | 657 | N | ARG | A | 91 | 4186 | 8939 | 4377 | 2237 | −81 | 412 | N |
| ATOM | 658 | CA | AARG | A | 91 | 83.954 | 33.786 | 0.170 | 0.60 | 45.39 | | C |
| ANISOU | 658 | CA | AARG | A | 91 | 3905 | 9157 | 4186 | 2390 | −57 | 372 | C |
| ATOM | 659 | CB | AARG | A | 91 | 84.257 | 34.000 | −1.336 | 0.60 | 43.40 | | C |
| ANISOU | 659 | CB | AARG | A | 91 | 3597 | 8978 | 3916 | 2425 | 29 | 421 | C |
| ATOM | 660 | CG | AARG | A | 91 | 85.757 | 34.138 | −1.677 | 0.60 | 42.54 | | C |
| ANISOU | 660 | CG | AARG | A | 91 | 3232 | 9219 | 3713 | 2535 | 84 | 374 | C |
| ATOM | 661 | CD | AARG | A | 91 | 86.048 | 34.309 | −3.161 | 0.60 | 38.80 | | C |
| ANISOU | 661 | CD | AARG | A | 91 | 2724 | 8811 | 3207 | 2579 | 178 | 425 | C |
| ATOM | 662 | NE | AARG | A | 91 | 85.718 | 35.640 | −3.683 | 0.60 | 28.09 | | N |
| ANISOU | 662 | NE | AARG | A | 91 | 1280 | 7395 | 2000 | 2309 | 309 | 473 | N |
| ATOM | 663 | CZ | AARG | A | 91 | 86.519 | 36.700 | −3.618 | 0.60 | 34.43 | | C |
| ANISOU | 663 | CZ | AARG | A | 91 | 1834 | 8393 | 2855 | 2132 | 429 | 440 | C |
| ATOM | 664 | NH1 | AARG | A | 91 | 87.697 | 36.616 | −3.011 | 0.60 | 25.01 | | N |
| ANISOU | 664 | NH1 | AARG | A | 91 | 972 | 7245 | 1286 | 1938 | 264 | 389 | N |
| ATOM | 665 | NH2 | AARG | A | 91 | 86.135 | 37.860 | −4.131 | 0.60 | 18.17 | | N |
| ANISOU | 665 | NH2 | AARG | A | 91 | 796 | 5659 | 450 | 1469 | 162 | 582 | N |
| ATOM | 666 | C | ARG | A | 91 | 84.680 | 34.832 | 0.994 | 1.00 | 50.64 | | C |
| ANISOU | 666 | C | ARG | A | 91 | 4296 | 10022 | 4923 | 2226 | −10 | 312 | C |
| ATOM | 667 | O | ARG | A | 91 | 85.763 | 34.551 | 1.511 | 1.00 | 51.09 | | O |
| ANISOU | 667 | O | ARG | A | 91 | 4208 | 10333 | 4871 | 2361 | −48 | 239 | O |
| ATOM | 668 | CA | BARG | A | 91 | 83.939 | 33.798 | 0.137 | 0.40 | 45.78 | | C |
| ANISOU | 668 | CA | BARG | A | 91 | 3955 | 9203 | 4236 | 2387 | −55 | 374 | C |
| ATOM | 669 | CB | BARG | A | 91 | 84.112 | 34.127 | −1.359 | 0.40 | 45.16 | | C |
| ANISOU | 669 | CB | BARG | A | 91 | 3824 | 9173 | 4160 | 2391 | 36 | 428 | C |
| ATOM | 670 | CG | BARG | A | 91 | 85.428 | 33.722 | −2.012 | 0.40 | 50.24 | | C |
| ANISOU | 670 | CG | BARG | A | 91 | 4327 | 10104 | 4659 | 2604 | 61 | 399 | C |
| ATOM | 671 | CD | BARG | A | 91 | 85.221 | 33.430 | −3.497 | 0.40 | 50.93 | | C |
| ANISOU | 671 | CD | BARG | A | 91 | 4525 | 10131 | 4696 | 2699 | 104 | 463 | C |
| ATOM | 672 | NE | BARG | A | 91 | 84.859 | 34.620 | −4.277 | 0.40 | 45.92 | | N |
| ANISOU | 672 | NE | BARG | A | 91 | 3812 | 9442 | 4192 | 2476 | 224 | 523 | N |
| ATOM | 673 | CZ | BARG | A | 91 | 83.884 | 34.665 | −5.183 | 0.40 | 46.89 | | C |
| ANISOU | 673 | CZ | BARG | A | 91 | 4106 | 9359 | 4350 | 2443 | 237 | 595 | C |
| ATOM | 674 | NH1 | BARG | A | 91 | 83.145 | 33.588 | −5.431 | 0.40 | 26.59 | | N |
| ANISOU | 674 | NH1 | BARG | A | 91 | 1782 | 6619 | 1702 | 2580 | 138 | 605 | N |
| ATOM | 675 | NH2 | BARG | A | 91 | 83.636 | 35.788 | −5.843 | 0.40 | 27.54 | | N |
| ANISOU | 675 | NH2 | BARG | A | 91 | 1593 | 6875 | 1997 | 2272 | 352 | 651 | N |
| ATOM | 676 | N | ARG | A | 92 | 84.085 | 36.032 | 1.131 | 1.00 | 48.10 | | N |
| ANISOU | 676 | N | ARG | A | 92 | 3912 | 9595 | 4771 | 1943 | 73 | 336 | N |
| ATOM | 677 | CA | ARG | A | 92 | 84.699 | 37.163 | 1.812 | 1.00 | 48.03 | | C |
| ANISOU | 677 | CA | ARG | A | 92 | 3668 | 9740 | 4840 | 1732 | 144 | 276 | C |
| ATOM | 678 | CB | ARG | A | 92 | 84.397 | 38.458 | 1.034 | 1.00 | 47.24 | | C |
| ANISOU | 678 | CB | ARG | A | 92 | 3518 | 9560 | 4872 | 1490 | 297 | 331 | C |
| ATOM | 679 | CG | ARG | A | 92 | 84.942 | 38.401 | −0.409 | 1.00 | 50.91 | | C |
| ANISOU | 679 | CG | ARG | A | 92 | 3935 | 10137 | 5273 | 1581 | 378 | 373 | C |
| ATOM | 680 | CD | ARG | A | 92 | 85.755 | 39.598 | −0.839 | 1.00 | 53.72 | | C |
| ANISOU | 680 | CD | ARG | A | 92 | 4087 | 10652 | 5672 | 1373 | 546 | 354 | C |
| ATOM | 681 | NE | ARG | A | 92 | 86.588 | 40.099 | 0.246 | 1.00 | 53.37 | | N |
| ANISOU | 681 | NE | ARG | A | 92 | 3825 | 10817 | 5637 | 1227 | 560 | 242 | N |
| ATOM | 682 | CZ | ARG | A | 92 | 86.784 | 41.384 | 0.498 | 1.00 | 73.89 | | C |
| ANISOU | 682 | CZ | ARG | A | 92 | 6321 | 13424 | 8329 | 929 | 696 | 211 | C |
| ATOM | 683 | NH1 | ARG | A | 92 | 86.269 | 42.312 | −0.299 | 1.00 | 65.16 | | N |
| ANISOU | 683 | NH1 | ARG | A | 92 | 5320 | 12131 | 7307 | 770 | 841 | 294 | N |
| ATOM | 684 | NH2 | ARG | A | 92 | 87.539 | 41.753 | 1.514 | 1.00 | 69.82 | | N |
| ANISOU | 684 | NH2 | ARG | A | 92 | 5606 | 13115 | 7805 | 795 | 694 | 92 | N |
| ATOM | 685 | C | ARG | A | 92 | 84.356 | 37.334 | 3.272 | 1.00 | 52.54 | | C |
| ANISOU | 685 | C | ARG | A | 92 | 4257 | 10244 | 5462 | 1631 | 82 | 226 | C |
| ATOM | 686 | O | ARG | A | 92 | 85.275 | 37.537 | 4.065 | 1.00 | 52.87 | | O |
| ANISOU | 686 | O | ARG | A | 92 | 4112 | 10521 | 5454 | 1623 | 64 | 135 | O |
| ATOM | 687 | N | PHE | A | 93 | 83.074 | 37.260 | 3.643 | 1.00 | 49.47 | | N |
| ANISOU | 687 | N | PHE | A | 93 | 4074 | 9564 | 5158 | 1557 | 50 | 274 | N |
| ATOM | 688 | CA | PHE | A | 93 | 82.640 | 37.504 | 5.028 | 1.00 | 49.30 | | C |
| ANISOU | 688 | CA | PHE | A | 93 | 4082 | 9457 | 5193 | 1443 | 7 | 232 | C |
| ATOM | 689 | CB | PHE | A | 93 | 81.633 | 38.663 | 5.024 | 1.00 | 50.14 | | C |
| ANISOU | 689 | CB | PHE | A | 93 | 4225 | 9355 | 5470 | 1188 | 98 | 279 | C |
| ATOM | 690 | CG | PHE | A | 93 | 82.157 | 39.908 | 4.352 | 1.00 | 50.52 | | C |
| ANISOU | 690 | CG | PHE | A | 93 | 4113 | 9492 | 5588 | 1015 | 242 | 287 | C |
| ATOM | 691 | CD1 | PHE | A | 93 | 83.029 | 40.761 | 5.016 | 1.00 | 52.99 | | C |
| ANISOU | 691 | CD1 | PHE | A | 93 | 4236 | 9974 | 5926 | 845 | 304 | 205 | C |
| ATOM | 692 | CE1 | PHE | A | 93 | 83.514 | 41.907 | 4.393 | 1.00 | 53.70 | | C |
| ANISOU | 692 | CE1 | PHE | A | 93 | 4209 | 10127 | 6068 | 656 | 460 | 206 | C |
| ATOM | 693 | CZ | PHE | A | 93 | 83.119 | 42.214 | 3.110 | 1.00 | 52.64 | | C |
| ANISOU | 693 | CZ | PHE | A | 93 | 4157 | 9884 | 5960 | 665 | 551 | 301 | C |
| ATOM | 694 | CE2 | PHE | A | 93 | 82.247 | 41.387 | 2.442 | 1.00 | 54.56 | | C |
| ANISOU | 694 | CE2 | PHE | A | 93 | 4572 | 9979 | 6178 | 853 | 475 | 384 | C |
| ATOM | 695 | CD2 | PHE | A | 93 | 81.774 | 40.230 | 3.059 | 1.00 | 51.85 | | C |
| ANISOU | 695 | CD2 | PHE | A | 93 | 4338 | 9578 | 5786 | 1015 | 323 | 371 | C |
| ATOM | 696 | C | PHE | A | 93 | 82.064 | 36.274 | 5.787 | 1.00 | 55.44 | | C |
| ANISOU | 696 | C | PHE | A | 93 | 5083 | 10097 | 5885 | 1608 | −119 | 228 | C |
| ATOM | 697 | O | PHE | A | 93 | 81.507 | 36.411 | 6.889 | 1.00 | 55.64 | | O |
| ANISOU | 697 | O | PHE | A | 93 | 5172 | 10011 | 5956 | 1516 | −150 | 206 | O |
| ATOM | 698 | N | GLY | A | 94 | 82.223 | 35.093 | 5.210 | 1.00 | 52.87 | | N |
| ANISOU | 698 | N | GLY | A | 94 | 4891 | 9774 | 5425 | 1847 | −178 | 247 | N |
| ATOM | 699 | CA | GLY | A | 94 | 81.711 | 33.875 | 5.816 | 1.00 | 53.13 | | C |
| ANISOU | 699 | CA | GLY | A | 94 | 5183 | 9652 | 5354 | 2001 | −274 | 245 | C |
| ATOM | 700 | C | GLY | A | 94 | 80.280 | 33.535 | 5.432 | 1.00 | 57.71 | | C |
| ANISOU | 700 | C | GLY | A | 94 | 6003 | 9932 | 5992 | 1918 | −272 | 303 | C |
| ATOM | 701 | O | GLY | A | 94 | 79.616 | 34.285 | 4.697 | 1.00 | 57.57 | | O |
| ANISOU | 701 | O | GLY | A | 94 | 5940 | 9833 | 6100 | 1756 | −208 | 350 | O |
| ATOM | 702 | N | PRO | A | 95 | 79.772 | 32.390 | 5.936 | 1.00 | 53.61 | | N |
| ANISOU | 702 | N | PRO | A | 95 | 5751 | 9252 | 5367 | 2028 | −338 | 297 | N |
| ATOM | 703 | CA | PRO | A | 95 | 78.421 | 31.963 | 5.555 | 1.00 | 52.88 | | C |
| ANISOU | 703 | CA | PRO | A | 95 | 5876 | 8904 | 5313 | 1925 | −334 | 332 | C |
| ATOM | 704 | CB | PRO | A | 95 | 78.461 | 30.453 | 5.753 | 1.00 | 54.97 | | C |
| ANISOU | 704 | CB | PRO | A | 95 | 6443 | 9054 | 5389 | 2121 | −393 | 314 | C |
| ATOM | 705 | CG | PRO | A | 95 | 79.466 | 30.240 | 6.819 | 1.00 | 59.79 | | C |
| ANISOU | 705 | CG | PRO | A | 95 | 7019 | 9807 | 5892 | 2291 | −437 | 273 | C |
| ATOM | 706 | CD | PRO | A | 95 | 80.430 | 31.389 | 6.802 | 1.00 | 55.39 | | C |
| ANISOU | 706 | CD | PRO | A | 95 | 6108 | 9526 | 5414 | 2256 | −412 | 254 | C |
| ATOM | 707 | C | PRO | A | 95 | 77.299 | 32.599 | 6.346 | 1.00 | 53.69 | | C |
| ANISOU | 707 | C | PRO | A | 95 | 5980 | 8862 | 5558 | 1685 | −310 | 333 | C |
| ATOM | 708 | O | PRO | A | 95 | 76.198 | 32.731 | 5.823 | 1.00 | 54.70 | | O |
| ANISOU | 708 | O | PRO | A | 95 | 6165 | 8856 | 5761 | 1554 | −286 | 361 | O |
| ATOM | 709 | N | THR | A | 96 | 77.560 | 32.974 | 7.584 | 1.00 | 46.69 | | N |
| ANISOU | 709 | N | THR | A | 96 | 5031 | 8016 | 4694 | 1641 | −321 | 298 | N |
| ATOM | 710 | CA | THR | A | 96 | 76.547 | 33.569 | 8.435 | 1.00 | 45.67 | | C |
| ANISOU | 710 | CA | THR | A | 96 | 4914 | 7752 | 4685 | 1433 | −296 | 295 | C |
| ATOM | 711 | CB | THR | A | 96 | 76.930 | 33.461 | 9.911 | 1.00 | 54.87 | | C |
| ANISOU | 711 | CB | THR | A | 96 | 6107 | 8941 | 5800 | 1460 | −333 | 246 | C |
| ATOM | 712 | OG1 | THR | A | 96 | 76.065 | 34.309 | 10.664 | 1.00 | 55.33 | | O |
| ANISOU | 712 | OG1 | THR | A | 96 | 6137 | 8895 | 5990 | 1247 | −294 | 244 | O |
| ATOM | 713 | CG2 | THR | A | 96 | 78.432 | 33.732 | 10.182 | 1.00 | 53.70 | | C |
| ANISOU | 713 | CG2 | THR | A | 96 | 5754 | 9056 | 5595 | 1577 | −357 | 203 | C |
| ATOM | 714 | C | THR | A | 96 | 76.192 | 34.984 | 7.958 | 1.00 | 47.22 | | C |
| ANISOU | 714 | C | THR | A | 96 | 4911 | 7977 | 5055 | 1248 | −220 | 326 | C |
| ATOM | 715 | O | THR | A | 96 | 75.013 | 35.329 | 7.945 | 1.00 | 47.82 | | O |
| ANISOU | 715 | O | THR | A | 96 | 5034 | 7914 | 5221 | 1108 | −190 | 349 | O |
| ATOM | 716 | N | VAL | A | 97 | 77.184 | 35.769 | 7.514 | 1.00 | 40.97 | | N |
| ANISOU | 716 | N | VAL | A | 97 | 3912 | 7361 | 4293 | 1255 | −178 | 327 | N |
| ATOM | 717 | CA | VAL | A | 97 | 76.935 | 37.105 | 6.970 | 1.00 | 39.65 | | C |
| ANISOU | 717 | CA | VAL | A | 97 | 3602 | 7197 | 4266 | 1095 | −85 | 364 | C |
| ATOM | 718 | CB | VAL | A | 97 | 78.265 | 37.869 | 6.771 | 1.00 | 43.11 | | C |
| ANISOU | 718 | CB | VAL | A | 97 | 3827 | 7848 | 4707 | 1085 | −32 | 339 | C |
| ATOM | 719 | CG1 | VAL | A | 97 | 78.121 | 39.016 | 5.780 | 1.00 | 43.46 | | C |
| ANISOU | 719 | CG1 | VAL | A | 97 | 3784 | 7882 | 4847 | 972 | 81 | 394 | C |
| ATOM | 720 | CG2 | VAL | A | 97 | 78.773 | 38.388 | 8.087 | 1.00 | 42.55 | | C |
| ANISOU | 720 | CG2 | VAL | A | 97 | 3657 | 7854 | 4658 | 989 | −33 | 270 | C |
| ATOM | 721 | C | VAL | A | 97 | 76.105 | 36.948 | 5.664 | 1.00 | 41.76 | | C |
| ANISOU | 721 | C | VAL | A | 97 | 3947 | 7373 | 4546 | 1114 | −68 | 428 | C |
| ATOM | 722 | O | VAL | A | 97 | 75.129 | 37.676 | 5.461 | 1.00 | 40.19 | | O |
| ANISOU | 722 | O | VAL | A | 97 | 3748 | 7076 | 4448 | 996 | −19 | 465 | O |
| ATOM | 723 | N | ARG | A | 98 | 76.471 | 35.953 | 4.824 | 1.00 | 37.74 | | N |
| ANISOU | 723 | N | ARG | A | 98 | 3513 | 6905 | 3921 | 1279 | −112 | 435 | N |
| ATOM | 724 | CA | ARG | A | 98 | 75.752 | 35.624 | 3.600 | 1.00 | 37.49 | | C |
| ANISOU | 724 | CA | ARG | A | 98 | 3569 | 6806 | 3870 | 1316 | −113 | 478 | C |
| ATOM | 725 | CB | ARG | A | 98 | 76.448 | 34.488 | 2.843 | 1.00 | 37.28 | | C |
| ANISOU | 725 | CB | ARG | A | 98 | 3636 | 6839 | 3691 | 1517 | −162 | 468 | C |
| ATOM | 726 | CG | ARG | A | 98 | 75.706 | 34.015 | 1.596 | 1.00 | 45.71 | | C |
| ANISOU | 726 | CG | ARG | A | 98 | 4806 | 7844 | 4718 | 1556 | −169 | 502 | C |
| ATOM | 727 | CD | ARG | A | 98 | 75.843 | 32.513 | 1.461 | 1.00 | 64.11 | | C |
| ANISOU | 727 | CD | ARG | A | 98 | 7360 | 10108 | 6889 | 1701 | −239 | 468 | C |
| ATOM | 728 | NE | ARG | A | 98 | 75.007 | 32.006 | 0.383 | 1.00 | 86.84 | | N |
| ANISOU | 728 | NE | ARG | A | 98 | 10364 | 12905 | 9726 | 1694 | −255 | 478 | N |
| ATOM | 729 | CZ | ARG | A | 98 | 75.395 | 31.157 | −0.562 | 1.00 | 109.59 | | C |
| ANISOU | 729 | CZ | ARG | A | 98 | 13364 | 15798 | 12475 | 1848 | −277 | 475 | C |
| ATOM | 730 | NH1 | ARG | A | 98 | 76.660 | 30.750 | −0.627 | 1.00 | 100.07 | | N |
| ANISOU | 730 | NH1 | ARG | A | 98 | 12150 | 14703 | 11169 | 2044 | −279 | 472 | N |
| ATOM | 731 | NH2 | ARG | A | 98 | 74.549 | 30.783 | −1.515 | 1.00 | 95.61 | | N |
| ANISOU | 731 | NH2 | ARG | A | 98 | 11697 | 13961 | 10669 | 1813 | −293 | 474 | N |
| ATOM | 732 | C | ARG | A | 98 | 74.316 | 35.236 | 3.922 | 1.00 | 42.48 | | C |
| ANISOU | 732 | C | ARG | A | 98 | 4346 | 7267 | 4529 | 1219 | −143 | 473 | C |
| ATOM | 733 | O | ARG | A | 98 | 73.404 | 35.781 | 3.302 | 1.00 | 43.23 | | O |
| ANISOU | 733 | O | ARG | A | 98 | 4412 | 7320 | 4694 | 1139 | −111 | 508 | O |
| ATOM | 734 | N | ARG | A | 99 | 74.113 | 34.321 | 4.898 | 1.00 | 38.05 | | N |
| ANISOU | 734 | N | ARG | A | 99 | 3936 | 6619 | 3901 | 1229 | −198 | 427 | N |
| ATOM | 735 | CA | ARG | A | 99 | 72.791 | 33.844 | 5.300 | 1.00 | 37.54 | | C |
| ANISOU | 735 | CA | ARG | A | 99 | 4015 | 6403 | 3845 | 1114 | −215 | 405 | C |
| ATOM | 736 | CB | ARG | A | 99 | 72.885 | 32.821 | 6.424 | 1.00 | 41.57 | | C |
| ANISOU | 736 | CB | ARG | A | 99 | 4715 | 6822 | 4258 | 1145 | −256 | 357 | C |
| ATOM | 737 | CG | ARG | A | 99 | 73.240 | 31.400 | 5.964 | 1.00 | 60.77 | | C |
| ANISOU | 737 | CG | ARG | A | 99 | 7375 | 9201 | 6514 | 1300 | −301 | 337 | C |
| ATOM | 738 | CD | ARG | A | 99 | 73.704 | 30.560 | 7.151 | 1.00 | 79.71 | | C |
| ANISOU | 738 | CD | ARG | A | 99 | 9959 | 11530 | 8797 | 1389 | −329 | 302 | C |
| ATOM | 739 | NE | ARG | A | 99 | 72.857 | 30.758 | 8.333 | 1.00 | 89.52 | | N |
| ANISOU | 739 | NE | ARG | A | 99 | 11250 | 12663 | 10102 | 1219 | −309 | 280 | N |
| ATOM | 740 | CZ | ARG | A | 99 | 73.312 | 30.980 | 9.563 | 1.00 | 102.29 | | C |
| ANISOU | 740 | CZ | ARG | A | 99 | 12840 | 14303 | 11723 | 1236 | −315 | 266 | C |
| ATOM | 741 | NH1 | ARG | A | 99 | 72.465 | 31.165 | 10.565 | 1.00 | 91.36 | | N |
| ANISOU | 741 | NH1 | ARG | A | 99 | 11507 | 12813 | 10392 | 1077 | −289 | 248 | N |
| ATOM | 742 | NH2 | ARG | A | 99 | 74.617 | 31.002 | 9.802 | 1.00 | 86.90 | | N |
| ANISOU | 742 | NH2 | ARG | A | 99 | 10804 | 12502 | 9712 | 1415 | −348 | 262 | N |
| ATOM | 743 | C | ARG | A | 99 | 71.875 | 34.981 | 5.694 | 1.00 | 38.26 | | C |
| ANISOU | 743 | C | ARG | A | 99 | 3988 | 6469 | 4082 | 946 | −166 | 421 | C |
| ATOM | 744 | O | ARG | A | 99 | 70.768 | 35.073 | 5.155 | 1.00 | 38.28 | | O |
| ANISOU | 744 | O | ARG | A | 99 | 3997 | 6434 | 4115 | 872 | −159 | 430 | O |
| ATOM | 745 | N | ILE | A | 100 | 72.362 | 35.886 | 6.563 | 1.00 | 31.94 | | N |
| ANISOU | 745 | N | ILE | A | 100 | 3073 | 5706 | 3358 | 898 | −130 | 421 | N |
| ATOM | 746 | CA | ILE | A | 100 | 71.621 | 37.075 | 6.996 | 1.00 | 30.79 | | C |
| ANISOU | 746 | CA | ILE | A | 100 | 2830 | 5528 | 3342 | 759 | −70 | 438 | C |
| ATOM | 747 | CB | ILE | A | 100 | 72.368 | 37.845 | 8.109 | 1.00 | 32.03 | | C |
| ANISOU | 747 | CB | ILE | A | 100 | 2901 | 5719 | 3551 | 708 | −39 | 415 | C |
| ATOM | 748 | CG1 | ILE | A | 100 | 72.614 | 36.968 | 9.340 | 1.00 | 31.04 | | C |
| ANISOU | 748 | CG1 | ILE | A | 100 | 2888 | 5557 | 3350 | 729 | −97 | 360 | C |
| ATOM | 749 | CD1 | ILE | A | 100 | 73.547 | 37.570 | 10.377 | 1.00 | 36.06 | | C |
| ANISOU | 749 | CD1 | ILE | A | 100 | 3433 | 6271 | 3998 | 712 | −90 | 322 | C |
| ATOM | 750 | CG2 | ILE | A | 100 | 71.620 | 39.114 | 8.470 | 1.00 | 32.38 | | C |
| ANISOU | 750 | CG2 | ILE | A | 100 | 2873 | 5711 | 3718 | 579 | 37 | 436 | C |
| ATOM | 751 | C | ILE | A | 100 | 71.259 | 38.010 | 5.800 | 1.00 | 38.67 | | C |
| ANISOU | 751 | C | ILE | A | 100 | 3728 | 6562 | 4401 | 758 | −14 | 498 | C |
| ATOM | 752 | O | ILE | A | 100 | 70.088 | 38.369 | 5.663 | 1.00 | 39.53 | | O |
| ANISOU | 752 | O | ILE | A | 100 | 3836 | 6628 | 4555 | 699 | 1 | 512 | O |
| ATOM | 753 | N | VAL | A | 101 | 72.259 | 38.403 | 4.955 | 1.00 | 34.98 | | N |
| ANISOU | 753 | N | VAL | A | 101 | 3180 | 6187 | 3922 | 832 | 19 | 531 | N |
| ATOM | 754 | CA | VAL | A | 101 | 72.089 | 39.332 | 3.828 | 1.00 | 34.48 | | C |
| ANISOU | 754 | CA | VAL | A | 101 | 3051 | 6151 | 3900 | 846 | 88 | 596 | C |
| ATOM | 755 | CB | VAL | A | 101 | 73.442 | 39.759 | 3.195 | 1.00 | 37.24 | | C |
| ANISOU | 755 | CB | VAL | A | 101 | 3314 | 6605 | 4231 | 897 | 145 | 617 | C |
| ATOM | 756 | CG1 | VAL | A | 101 | 73.256 | 40.455 | 1.853 | 1.00 | 36.11 | | C |
| ANISOU | 756 | CG1 | VAL | A | 101 | 3154 | 6476 | 4092 | 941 | 214 | 691 | C |
| ATOM | 757 | CG2 | VAL | A | 101 | 74.241 | 40.645 | 4.150 | 1.00 | 36.82 | | C |
| ANISOU | 757 | CG2 | VAL | A | 101 | 3169 | 6581 | 4240 | 789 | 212 | 590 | C |
| ATOM | 758 | C | VAL | A | 101 | 71.083 | 38.777 | 2.826 | 1.00 | 42.09 | | C |
| ANISOU | 758 | C | VAL | A | 101 | 4077 | 7099 | 4815 | 902 | 45 | 615 | C |
| ATOM | 759 | O | VAL | A | 101 | 70.171 | 39.510 | 2.427 | 1.00 | 42.48 | | O |
| ANISOU | 759 | O | VAL | A | 101 | 4098 | 7133 | 4908 | 884 | 81 | 653 | O |
| ATOM | 760 | N | GLU | A | 102 | 71.203 | 37.474 | 2.472 | 1.00 | 40.18 | | N |
| ANISOU | 760 | N | GLU | A | 102 | 3931 | 6865 | 4471 | 971 | −34 | 580 | N |
| ATOM | 761 | CA | GLU | A | 102 | 70.251 | 36.789 | 1.595 | 1.00 | 40.06 | | C |
| ANISOU | 761 | CA | GLU | A | 102 | 3986 | 6842 | 4392 | 995 | −84 | 573 | C |
| ATOM | 762 | CB | GLU | A | 102 | 70.698 | 35.359 | 1.298 | 1.00 | 41.42 | | C |
| ANISOU | 762 | CB | GLU | A | 102 | 4299 | 7002 | 4436 | 1073 | −153 | 530 | C |
| ATOM | 763 | CG | GLU | A | 102 | 71.425 | 35.247 | −0.026 | 1.00 | 55.75 | | C |
| ANISOU | 763 | CG | GLU | A | 102 | 6107 | 8895 | 6181 | 1212 | −150 | 567 | C |
| ATOM | 764 | CD | GLU | A | 102 | 71.805 | 33.841 | −0.448 | 1.00 | 84.59 | | C |
| ANISOU | 764 | CD | GLU | A | 102 | 9925 | 12525 | 9690 | 1310 | −212 | 525 | C |
| ATOM | 765 | OE1 | GLU | A | 102 | 72.342 | 33.083 | 0.396 | 1.00 | 86.78 | | O |
| ANISOU | 765 | OE1 | GLU | A | 102 | 10312 | 12748 | 9911 | 1333 | −240 | 483 | O |
| ATOM | 766 | OE2 | GLU | A | 102 | 71.586 | 33.506 | −1.636 | 1.00 | 74.59 | | O |
| ANISOU | 766 | OE2 | GLU | A | 102 | 8696 | 11292 | 8354 | 1377 | −229 | 536 | O |
| ATOM | 767 | C | GLU | A | 102 | 68.886 | 36.786 | 2.281 | 1.00 | 44.88 | | C |
| ANISOU | 767 | C | GLU | A | 102 | 4608 | 7400 | 5043 | 873 | −100 | 538 | C |
| ATOM | 768 | O | GLU | A | 102 | 67.915 | 37.254 | 1.696 | 1.00 | 44.10 | | O |
| ANISOU | 768 | O | GLU | A | 102 | 4452 | 7340 | 4963 | 865 | −91 | 558 | O |
| ATOM | 769 | N | GLY | A | 103 | 68.859 | 36.371 | 3.547 | 1.00 | 42.48 | | N |
| ANISOU | 769 | N | GLY | A | 103 | 4366 | 7025 | 4750 | 789 | −114 | 489 | N |
| ATOM | 770 | CA | GLY | A | 103 | 67.652 | 36.350 | 4.370 | 1.00 | 42.81 | | C |
| ANISOU | 770 | CA | GLY | A | 103 | 4417 | 7020 | 4828 | 657 | −114 | 448 | C |
| ATOM | 771 | C | GLY | A | 103 | 66.854 | 37.646 | 4.398 | 1.00 | 46.64 | | C |
| ANISOU | 771 | C | GLY | A | 103 | 4766 | 7542 | 5411 | 624 | −58 | 486 | C |
| ATOM | 772 | O | GLY | A | 103 | 65.639 | 37.615 | 4.189 | 1.00 | 46.62 | | O |
| ANISOU | 772 | O | GLY | A | 103 | 4728 | 7584 | 5403 | 580 | −70 | 464 | O |
| ATOM | 773 | N | GLU | A | 104 | 67.526 | 38.788 | 4.654 | 1.00 | 42.71 | | N |
| ANISOU | 773 | N | GLU | A | 104 | 4199 | 7037 | 4992 | 648 | 8 | 536 | N |
| ATOM | 774 | CA | GLU | A | 104 | 66.900 | 40.109 | 4.709 | 1.00 | 42.95 | | C |
| ANISOU | 774 | CA | GLU | A | 104 | 4144 | 7072 | 5102 | 641 | 80 | 581 | C |
| ATOM | 775 | CB | GLU | A | 104 | 67.860 | 41.160 | 5.332 | 1.00 | 44.22 | | C |
| ANISOU | 775 | CB | GLU | A | 104 | 4279 | 7187 | 5335 | 620 | 162 | 611 | C |
| ATOM | 776 | CG | GLU | A | 104 | 67.307 | 42.584 | 5.476 | 1.00 | 55.21 | | C |
| ANISOU | 776 | CG | GLU | A | 104 | 5636 | 8542 | 6800 | 613 | 257 | 656 | C |
| ATOM | 777 | CD | GLU | A | 104 | 67.287 | 43.438 | 4.212 | 1.00 | 80.35 | | C |
| ANISOU | 777 | CD | GLU | A | 104 | 8810 | 11749 | 9972 | 721 | 322 | 737 | C |
| ATOM | 778 | OE1 | GLU | A | 104 | 68.312 | 43.443 | 3.491 | 1.00 | 86.62 | | O |
| ANISOU | 778 | OE1 | GLU | A | 104 | 9608 | 12564 | 10739 | 760 | 343 | 766 | O |
| ATOM | 779 | OE2 | GLU | A | 104 | 66.248 | 44.081 | 3.926 | 1.00 | 66.67 | | O |
| ANISOU | 779 | OE2 | GLU | A | 104 | 7068 | 10021 | 8243 | 781 | 354 | 771 | O |
| ATOM | 780 | C | GLU | A | 104 | 66.411 | 40.533 | 3.315 | 1.00 | 49.79 | | C |
| ANISOU | 780 | C | GLU | A | 104 | 4967 | 8014 | 5936 | 749 | 89 | 634 | C |
| ATOM | 781 | O | GLU | A | 104 | 65.333 | 41.135 | 3.211 | 1.00 | 50.80 | | O |
| ANISOU | 781 | O | GLU | A | 104 | 5044 | 8177 | 6080 | 769 | 110 | 650 | O |
| ATOM | 782 | N | THR | A | 105 | 67.190 | 40.246 | 2.254 | 1.00 | 46.42 | | N |
| ANISOU | 782 | N | THR | A | 105 | 4561 | 7626 | 5452 | 837 | 73 | 662 | N |
| ATOM | 783 | CA | THR | A | 105 | 66.797 | 40.662 | 0.902 | 1.00 | 46.82 | | C |
| ANISOU | 783 | CA | THR | A | 105 | 4586 | 7748 | 5455 | 954 | 81 | 717 | C |
| ATOM | 784 | CB | THR | A | 105 | 68.021 | 40.736 | −0.031 | 1.00 | 59.06 | | C |
| ANISOU | 784 | CB | THR | A | 105 | 6160 | 9315 | 6967 | 1043 | 110 | 765 | C |
| ATOM | 785 | OG1 | THR | A | 105 | 68.972 | 41.647 | 0.533 | 1.00 | 60.09 | | O |
| ANISOU | 785 | OG1 | THR | A | 105 | 6274 | 9392 | 7166 | 998 | 203 | 794 | O |
| ATOM | 786 | CG2 | THR | A | 105 | 67.668 | 41.228 | −1.416 | 1.00 | 59.99 | | C |
| ANISOU | 786 | CG2 | THR | A | 105 | 6272 | 9492 | 7031 | 1174 | 136 | 834 | C |
| ATOM | 787 | C | THR | A | 105 | 65.629 | 39.798 | 0.376 | 1.00 | 50.08 | | C |
| ANISOU | 787 | C | THR | A | 105 | 4990 | 8244 | 5792 | 956 | −10 | 663 | C |
| ATOM | 788 | O | THR | A | 105 | 64.676 | 40.344 | −0.197 | 1.00 | 49.25 | | O |
| ANISOU | 788 | O | THR | A | 105 | 4825 | 8222 | 5667 | 1023 | −8 | 684 | O |
| ATOM | 789 | N | LYS | A | 106 | 65.685 | 38.474 | 0.635 | 1.00 | 46.33 | | N |
| ANISOU | 789 | N | LYS | A | 106 | 4585 | 7754 | 5263 | 880 | −83 | 586 | N |
| ATOM | 790 | CA | LYS | A | 106 | 64.683 | 37.493 | 0.219 | 1.00 | 45.85 | | C |
| ANISOU | 790 | CA | LYS | A | 106 | 4541 | 7763 | 5118 | 828 | −162 | 510 | C |
| ATOM | 791 | CB | LYS | A | 106 | 65.172 | 36.067 | 0.467 | 1.00 | 46.49 | | C |
| ANISOU | 791 | CB | LYS | A | 106 | 4769 | 7768 | 5126 | 762 | −212 | 442 | C |
| ATOM | 792 | CG | LYS | A | 106 | 66.137 | 35.595 | −0.597 | 1.00 | 54.90 | | C |
| ANISOU | 792 | CG | LYS | A | 106 | 5905 | 8836 | 6116 | 890 | −231 | 473 | C |
| ATOM | 793 | CD | LYS | A | 106 | 66.745 | 34.263 | −0.266 | 1.00 | 60.73 | | C |
| ANISOU | 793 | CD | LYS | A | 106 | 6818 | 9481 | 6775 | 866 | −266 | 418 | C |
| ATOM | 794 | CE | LYS | A | 106 | 67.637 | 33.797 | −1.389 | 1.00 | 73.46 | | C |
| ANISOU | 794 | CE | LYS | A | 106 | 8503 | 11117 | 8293 | 1007 | −288 | 439 | C |
| ATOM | 795 | NZ | LYS | A | 106 | 67.969 | 32.357 | −1.264 | 1.00 | 86.72 | | N |
| ANISOU | 795 | NZ | LYS | A | 106 | 10392 | 12702 | 9854 | 997 | −329 | 372 | N |
| ATOM | 796 | C | LYS | A | 106 | 63.308 | 37.702 | 0.836 | 1.00 | 52.73 | | C |
| ANISOU | 796 | C | LYS | A | 106 | 5328 | 8693 | 6016 | 727 | −165 | 458 | C |
| ATOM | 797 | O | LYS | A | 106 | 62.321 | 37.516 | 0.118 | 1.00 | 52.38 | | O |
| ANISOU | 797 | O | LYS | A | 106 | 5216 | 8780 | 5904 | 729 | −212 | 416 | O |
| ATOM | 798 | N | VAL | A | 107 | 63.243 | 38.076 | 2.158 | 1.00 | 50.92 | | N |
| ANISOU | 798 | N | VAL | A | 107 | 5088 | 8385 | 5873 | 640 | −117 | 453 | N |
| ATOM | 799 | CA | VAL | A | 107 | 62.008 | 38.318 | 2.932 | 1.00 | 51.46 | | C |
| ANISOU | 799 | CA | VAL | A | 107 | 5071 | 8508 | 5972 | 545 | −103 | 404 | C |
| ATOM | 800 | CB | VAL | A | 107 | 62.225 | 38.380 | 4.474 | 1.00 | 54.91 | | C |
| ANISOU | 800 | CB | VAL | A | 107 | 5555 | 8823 | 6487 | 434 | −55 | 388 | C |
| ATOM | 801 | CG1 | VAL | A | 107 | 62.875 | 39.683 | 4.920 | 1.00 | 54.35 | | C |
| ANISOU | 801 | CG1 | VAL | A | 107 | 5458 | 8685 | 6509 | 515 | 22 | 472 | C |
| ATOM | 802 | CG2 | VAL | A | 107 | 60.918 | 38.152 | 5.220 | 1.00 | 54.72 | | C |
| ANISOU | 802 | CG2 | VAL | A | 107 | 5468 | 8862 | 6460 | 299 | −50 | 311 | C |
| ATOM | 803 | C | VAL | A | 107 | 61.228 | 39.535 | 2.412 | 1.00 | 58.92 | | C |
| ANISOU | 803 | C | VAL | A | 107 | 5878 | 9579 | 6931 | 673 | −74 | 456 | C |
| ATOM | 804 | O | VAL | A | 107 | 59.998 | 39.471 | 2.318 | 1.00 | 59.17 | | O |
| ANISOU | 804 | O | VAL | A | 107 | 5802 | 9758 | 6921 | 646 | −102 | 399 | O |
| ATOM | 805 | N | SER | A | 108 | 61.945 | 40.624 | 2.060 | 1.00 | 57.53 | | N |
| ANISOU | 805 | N | SER | A | 108 | 5713 | 9350 | 6796 | 815 | −12 | 558 | N |
| ATOM | 806 | CA | SER | A | 108 | 61.382 | 41.868 | 1.522 | 1.00 | 58.02 | | C |
| ANISOU | 806 | CA | SER | A | 108 | 5706 | 9485 | 6854 | 979 | 36 | 630 | C |
| ATOM | 807 | CB | SER | A | 108 | 62.507 | 42.817 | 1.103 | 1.00 | 63.26 | | C |
| ANISOU | 807 | CB | SER | A | 108 | 6451 | 10035 | 7549 | 1087 | 121 | 737 | C |
| ATOM | 808 | OG | SER | A | 108 | 63.470 | 43.023 | 2.130 | 1.00 | 72.09 | | O |
| ANISOU | 808 | OG | SER | A | 108 | 7634 | 11003 | 8754 | 978 | 177 | 741 | O |
| ATOM | 809 | C | SER | A | 108 | 60.459 | 41.560 | 0.331 | 1.00 | 60.91 | | C |
| ANISOU | 809 | C | SER | A | 108 | 5985 | 10050 | 7108 | 1075 | −40 | 603 | C |
| ATOM | 810 | O | SER | A | 108 | 59.357 | 42.106 | 0.239 | 1.00 | 60.81 | | O |
| ANISOU | 810 | O | SER | A | 108 | 5863 | 10180 | 7063 | 1159 | −41 | 596 | O |
| ATOM | 811 | N | LYS | A | 109 | 60.892 | 40.603 | −0.510 | 1.00 | 56.59 | | N |
| ANISOU | 811 | N | LYS | A | 109 | 5483 | 9528 | 6492 | 1060 | −108 | 575 | N |
| ATOM | 812 | CA | LYS | A | 109 | 60.222 | 40.100 | −1.715 | 1.00 | 56.06 | | C |
| ANISOU | 812 | CA | LYS | A | 109 | 5355 | 9644 | 6302 | 1126 | −194 | 533 | C |
| ATOM | 813 | CB | LYS | A | 109 | 61.246 | 39.382 | −2.627 | 1.00 | 58.94 | | C |
| ANISOU | 813 | CB | LYS | A | 109 | 5832 | 9950 | 6611 | 1150 | −229 | 547 | C |
| ATOM | 814 | CG | LYS | A | 109 | 62.570 | 40.129 | −2.832 | 1.00 | 71.26 | | C |
| ANISOU | 814 | CG | LYS | A | 109 | 7487 | 11362 | 8226 | 1251 | −143 | 663 | C |
| ATOM | 815 | CD | LYS | A | 109 | 63.580 | 39.267 | −3.576 | 1.00 | 86.33 | | C |
| ANISOU | 815 | CD | LYS | A | 109 | 9497 | 13228 | 10078 | 1263 | −175 | 661 | C |
| ATOM | 816 | CE | LYS | A | 109 | 64.891 | 39.986 | −3.813 | 1.00 | 103.97 | | C |
| ANISOU | 816 | CE | LYS | A | 109 | 11789 | 15360 | 12356 | 1352 | −81 | 764 | C |
| ATOM | 817 | NZ | LYS | A | 109 | 65.936 | 39.110 | −4.435 | 1.00 | 111.85 | | N |
| ANISOU | 817 | NZ | LYS | A | 109 | 12869 | 16335 | 13293 | 1379 | −105 | 761 | N |
| ATOM | 818 | C | LYS | A | 109 | 59.016 | 39.169 | −1.427 | 1.00 | 57.71 | | C |
| ANISOU | 818 | C | LYS | A | 109 | 5466 | 10005 | 6458 | 962 | −270 | 393 | C |
| ATOM | 819 | O | LYS | A | 109 | 58.142 | 39.040 | −2.268 | 1.00 | 57.05 | | O |
| ANISOU | 819 | O | LYS | A | 109 | 5269 | 10134 | 6273 | 1017 | −334 | 343 | O |
| ATOM | 820 | N | LEU | A | 110 | 59.008 | 38.483 | −0.282 | 1.00 | 53.85 | | N |
| ANISOU | 820 | N | LEU | A | 110 | 5026 | 9414 | 6021 | 754 | −258 | 323 | N |
| ATOM | 821 | CA | LEU | A | 110 | 57.935 | 37.575 | 0.122 | 1.00 | 53.17 | | C |
| ANISOU | 821 | CA | LEU | A | 110 | 4875 | 9441 | 5886 | 550 | −300 | 185 | C |
| ATOM | 822 | CB | LEU | A | 110 | 58.405 | 36.659 | 1.246 | 1.00 | 52.52 | | C |
| ANISOU | 822 | CB | LEU | A | 110 | 4945 | 9165 | 5846 | 341 | −272 | 135 | C |
| ATOM | 823 | CG | LEU | A | 110 | 58.749 | 35.241 | 0.875 | 1.00 | 56.21 | | C |
| ANISOU | 823 | CG | LEU | A | 110 | 5573 | 9563 | 6222 | 216 | −321 | 58 | C |
| ATOM | 824 | CD1 | LEU | A | 110 | 59.913 | 35.180 | −0.097 | 1.00 | 56.15 | | C |
| ANISOU | 824 | CD1 | LEU | A | 110 | 5668 | 9483 | 6185 | 382 | −346 | 136 | C |
| ATOM | 825 | CD2 | LEU | A | 110 | 59.140 | 34.491 | 2.097 | 1.00 | 58.92 | | C |
| ANISOU | 825 | CD2 | LEU | A | 110 | 6086 | 9708 | 6592 | 52 | −281 | 23 | C |
| ATOM | 826 | C | LEU | A | 110 | 56.779 | 38.384 | 0.605 | 1.00 | 59.19 | | C |
| ANISOU | 826 | C | LEU | A | 110 | 5452 | 10372 | 6667 | 579 | −275 | 167 | C |
| ATOM | 827 | O | LEU | A | 110 | 55.633 | 37.990 | 0.394 | 1.00 | 58.55 | | O |
| ANISOU | 827 | O | LEU | A | 110 | 5221 | 10525 | 6502 | 516 | −326 | 65 | O |
| ATOM | 828 | N | TYR | A | 111 | 57.074 | 39.524 | 1.255 | 1.00 | 58.01 | | N |
| ANISOU | 828 | N | TYR | A | 111 | 5306 | 10120 | 6615 | 682 | −195 | 261 | N |
| ATOM | 829 | CA | TYR | A | 111 | 56.044 | 40.435 | 1.723 | 1.00 | 59.12 | | C |
| ANISOU | 829 | CA | TYR | A | 111 | 5291 | 10404 | 6767 | 760 | −158 | 261 | C |
| ATOM | 830 | CB | TYR | A | 111 | 56.556 | 41.364 | 2.822 | 1.00 | 58.58 | | C |
| ANISOU | 830 | CB | TYR | A | 111 | 5301 | 10140 | 6818 | 796 | −56 | 346 | C |
| ATOM | 831 | CG | TYR | A | 111 | 56.603 | 40.677 | 4.170 | 1.00 | 58.17 | | C |
| ANISOU | 831 | CG | TYR | A | 111 | 5303 | 9970 | 6829 | 558 | −28 | 276 | C |
| ATOM | 832 | CD1 | TYR | A | 111 | 55.504 | 39.969 | 4.658 | 1.00 | 59.08 | | C |
| ANISOU | 832 | CD1 | TYR | A | 111 | 5310 | 10233 | 6904 | 384 | −46 | 154 | C |
| ATOM | 833 | CE1 | TYR | A | 111 | 55.553 | 39.320 | 5.888 | 1.00 | 58.11 | | C |
| ANISOU | 833 | CE1 | TYR | A | 111 | 5269 | 9986 | 6825 | 166 | −7 | 96 | C |
| ATOM | 834 | CZ | TYR | A | 111 | 56.709 | 39.380 | 6.652 | 1.00 | 61.72 | | C |
| ANISOU | 834 | CZ | TYR | A | 111 | 5904 | 10186 | 7362 | 143 | 34 | 157 | C |
| ATOM | 835 | OH | TYR | A | 111 | 56.775 | 38.785 | 7.882 | 1.00 | 57.44 | | O |
| ANISOU | 835 | OH | TYR | A | 111 | 5457 | 9520 | 6846 | −42 | 72 | 105 | O |
| ATOM | 836 | CE2 | TYR | A | 111 | 57.809 | 40.078 | 6.189 | 1.00 | 58.61 | | C |
| ANISOU | 836 | CE2 | TYR | A | 111 | 5588 | 9673 | 7009 | 309 | 43 | 265 | C |
| ATOM | 837 | CD2 | TYR | A | 111 | 57.751 | 40.719 | 4.954 | 1.00 | 58.43 | | C |
| ANISOU | 837 | CD2 | TYR | A | 111 | 5495 | 9757 | 6947 | 503 | 20 | 325 | C |
| ATOM | 838 | C | TYR | A | 111 | 55.367 | 41.190 | 0.562 | 1.00 | 70.46 | | C |
| ANISOU | 838 | C | TYR | A | 111 | 6594 | 12073 | 8107 | 1010 | −195 | 296 | C |
| ATOM | 839 | O | TYR | A | 111 | 54.214 | 41.626 | 0.727 | 1.00 | 70.23 | | O |
| ANISOU | 839 | O | TYR | A | 111 | 6386 | 12268 | 8031 | 1065 | −202 | 247 | O |
| ATOM | 840 | N | LYS | A | 112 | 56.046 | 41.273 | −0.635 | 1.00 | 71.55 | | N |
| ANISOU | 840 | N | LYS | A | 112 | 6813 | 12179 | 8196 | 1165 | −222 | 371 | N |
| ATOM | 841 | CA | LYS | A | 112 | 55.476 | 41.885 | −1.857 | 1.00 | 73.17 | | C |
| ANISOU | 841 | CA | LYS | A | 112 | 6925 | 12594 | 8283 | 1422 | −262 | 409 | C |
| ATOM | 842 | CB | LYS | A | 112 | 56.538 | 42.379 | −2.867 | 1.00 | 76.13 | | C |
| ANISOU | 842 | CB | LYS | A | 112 | 7455 | 12835 | 8635 | 1587 | −245 | 528 | C |
| ATOM | 843 | CG | LYS | A | 112 | 57.357 | 43.594 | −2.385 | 1.00 | 98.26 | | C |
| ANISOU | 843 | CG | LYS | A | 112 | 10401 | 15415 | 11520 | 1731 | −116 | 676 | C |
| ATOM | 844 | CD | LYS | A | 112 | 56.681 | 44.959 | −2.636 | 1.00 | 109.74 | | C |
| ANISOU | 844 | CD | LYS | A | 112 | 11813 | 16967 | 12915 | 2003 | −62 | 752 | C |
| ATOM | 845 | CE | LYS | A | 112 | 56.657 | 45.855 | −1.408 | 1.00 | 120.42 | | C |
| ANISOU | 845 | CE | LYS | A | 112 | 13224 | 18154 | 14375 | 1989 | 55 | 799 | C |
| ATOM | 846 | NZ | LYS | A | 112 | 57.980 | 46.472 | −1.133 | 1.00 | 127.43 | | N |
| ANISOU | 846 | NZ | LYS | A | 112 | 14321 | 18744 | 15353 | 1969 | 172 | 905 | N |
| ATOM | 847 | C | LYS | A | 112 | 54.442 | 40.963 | −2.515 | 1.00 | 80.71 | | C |
| ANISOU | 847 | C | LYS | A | 112 | 7706 | 13854 | 9107 | 1348 | −380 | 267 | C |
| ATOM | 848 | O | LYS | A | 112 | 53.480 | 41.466 | −3.079 | 1.00 | 81.00 | | O |
| ANISOU | 848 | O | LYS | A | 112 | 7580 | 14160 | 9037 | 1532 | −423 | 251 | O |
| ATOM | 849 | N | LEU | A | 113 | 54.604 | 39.626 | −2.395 | 1.00 | 79.99 | | N |
| ANISOU | 849 | N | LEU | A | 113 | 7659 | 13724 | 9010 | 1082 | −429 | 157 | N |
| ATOM | 850 | CA | LEU | A | 113 | 53.661 | 38.619 | −2.900 | 1.00 | 81.44 | | C |
| ANISOU | 850 | CA | LEU | A | 113 | 7708 | 14167 | 9069 | 936 | −529 | −3 | C |
| ATOM | 851 | CB | LEU | A | 113 | 54.340 | 37.252 | −2.951 | 1.00 | 80.69 | | C |
| ANISOU | 851 | CB | LEU | A | 113 | 7791 | 13897 | 8970 | 692 | −554 | −72 | C |
| ATOM | 852 | CG | LEU | A | 113 | 54.720 | 36.784 | −4.326 | 1.00 | 84.24 | | C |
| ANISOU | 852 | CG | LEU | A | 113 | 8309 | 14390 | 9309 | 776 | −631 | −74 | C |
| ATOM | 853 | CD2 | LEU | A | 113 | 55.011 | 35.331 | −4.306 | 1.00 | 85.67 | | C |
| ANISOU | 853 | CD2 | LEU | A | 113 | 8679 | 14401 | 9470 | 540 | −646 | −154 | C |
| ATOM | 854 | CD1 | LEU | A | 113 | 55.900 | 37.565 | −4.884 | 1.00 | 84.33 | | C |
| ANISOU | 854 | CD1 | LEU | A | 113 | 8418 | 14278 | 9347 | 1070 | −597 | 101 | C |
| ATOM | 855 | C | LEU | A | 113 | 52.403 | 38.562 | −2.023 | 1.00 | 92.97 | | C |
| ANISOU | 855 | C | LEU | A | 113 | 8950 | 15860 | 10515 | 801 | −527 | −125 | C |
| ATOM | 856 | O | LEU | A | 113 | 51.288 | 38.340 | −2.519 | 1.00 | 91.59 | | O |
| ANISOU | 856 | O | LEU | A | 113 | 8556 | 16029 | 10214 | 813 | −603 | −235 | O |
| ATOM | 857 | N | ALA | A | 114 | 52.600 | 38.750 | −0.716 | 1.00 | 96.63 | | N |
| ANISOU | 857 | N | ALA | A | 114 | 9466 | 16147 | 11101 | 678 | −437 | −108 | N |
| ATOM | 858 | CA | ALA | A | 114 | 51.464 | 38.849 | 0.228 | 1.00 | 99.68 | | C |
| ANISOU | 858 | CA | ALA | A | 114 | 9682 | 16686 | 11505 | 557 | −398 | −194 | C |
| ATOM | 859 | CB | ALA | A | 114 | 51.976 | 38.911 | 1.643 | 1.00 | 100.40 | | C |
| ANISOU | 859 | CB | ALA | A | 114 | 9934 | 16474 | 11740 | 434 | −297 | −141 | C |
| ATOM | 860 | C | ALA | A | 114 | 50.689 | 40.116 | −0.145 | 1.00 | 111.78 | | C |
| ANISOU | 860 | C | ALA | A | 114 | 11020 | 18459 | 12992 | 870 | −394 | −137 | C |
| ATOM | 861 | O | ALA | A | 114 | 49.449 | 40.113 | −0.055 | 1.00 | 111.67 | | O |
| ANISOU | 861 | O | ALA | A | 114 | 10760 | 18762 | 12908 | 843 | −413 | −246 | O |
| ATOM | 862 | O | ASN | A | 115 | 50.242 | 43.027 | 1.274 | 1.00 | 125.17 | | O |
| ANISOU | 862 | O | ASN | A | 115 | 12717 | 20027 | 14815 | 1396 | −188 | 114 | O |
| ATOM | 863 | N | ASN | A | 115 | 51.434 | 41.170 | −0.496 | 1.00 | 113.89 | | N |
| ANISOU | 863 | N | ASN | A | 115 | 11412 | 18573 | 13289 | 1169 | −360 | 32 | N |
| ATOM | 864 | CA | ASN | A | 115 | 50.899 | 42.482 | −0.952 | 1.00 | 116.10 | | C |
| ANISOU | 864 | CA | ASN | A | 115 | 11618 | 18981 | 13514 | 1534 | −336 | 133 | C |
| ATOM | 865 | C | ASN | A | 115 | 49.916 | 43.047 | 0.074 | 1.00 | 125.00 | | C |
| ANISOU | 865 | C | ASN | A | 115 | 12594 | 20236 | 14666 | 1553 | −274 | 97 | C |
| ATOM | 866 | CB | ASN | A | 115 | 50.303 | 42.424 | −2.361 | 1.00 | 117.77 | | C |
| ANISOU | 866 | CB | ASN | A | 115 | 11676 | 19526 | 13545 | 1734 | −449 | 94 | C |
| ATOM | 867 | O | LEU | A | 116 | 46.556 | 42.016 | 0.659 | 1.00 | 129.36 | | O |
| ANISOU | 867 | O | LEU | A | 116 | 12351 | 21833 | 14966 | 1267 | −400 | −345 | O |
| ATOM | 868 | N | LEU | A | 116 | 48.752 | 43.512 | −0.394 | 1.00 | 124.08 | | N |
| ANISOU | 868 | N | LEU | A | 116 | 12223 | 20508 | 14411 | 1748 | −323 | 39 | N |
| ATOM | 869 | CA | LEU | A | 116 | 47.734 | 44.092 | 0.520 | 1.00 | 124.57 | | C |
| ANISOU | 869 | CA | LEU | A | 116 | 12093 | 20778 | 14460 | 1802 | −275 | −12 | C |
| ATOM | 870 | C | LEU | A | 116 | 47.077 | 42.950 | 1.298 | 1.00 | 129.16 | | C |
| ANISOU | 870 | C | LEU | A | 116 | 12495 | 21530 | 15048 | 1408 | −301 | −205 | C |
| ATOM | 871 | CB | LEU | A | 116 | 46.697 | 44.858 | −0.307 | 1.00 | 124.61 | | C |
| ANISOU | 871 | CB | LEU | A | 116 | 11887 | 21170 | 14288 | 2188 | −325 | −8 | C |
| ATOM | 872 | O | ALA | A | 117 | 44.454 | 43.209 | 3.241 | 1.00 | 126.06 | | O |
| ANISOU | 872 | O | ALA | A | 117 | 11557 | 21742 | 14597 | 1261 | −207 | −446 | O |
| ATOM | 873 | N | ALA | A | 117 | 47.100 | 43.048 | 2.627 | 1.00 | 124.76 | | N |
| ANISOU | 873 | N | ALA | A | 117 | 12003 | 20787 | 14613 | 1218 | −202 | −212 | N |
| ATOM | 874 | CA | ALA | A | 117 | 46.513 | 42.014 | 3.507 | 1.00 | 123.96 | | C |
| ANISOU | 874 | CA | ALA | A | 117 | 11784 | 20784 | 14530 | 842 | −183 | −374 | C |
| ATOM | 875 | C | ALA | A | 117 | 44.985 | 42.112 | 3.488 | 1.00 | 126.61 | | C |
| ANISOU | 875 | C | ALA | A | 117 | 11750 | 21622 | 14735 | 895 | −211 | −510 | C |
| ATOM | 876 | CB | ALA | A | 117 | 47.062 | 42.163 | 4.904 | 1.00 | 124.65 | | C |
| ANISOU | 876 | CB | ALA | A | 117 | 12052 | 20542 | 14767 | 712 | −62 | −318 | C |
| ATOM | 877 | O | GLY | A | 118 | 41.348 | 42.463 | 4.799 | 1.00 | 123.42 | | O |
| ANISOU | 877 | O | GLY | A | 118 | 10434 | 22369 | 14093 | 902 | −154 | −858 | O |
| ATOM | 878 | N | GLY | A | 118 | 44.322 | 40.982 | 3.728 | 1.00 | 122.10 | | N |
| ANISOU | 878 | N | GLY | A | 118 | 11013 | 21267 | 14114 | 536 | −232 | −701 | N |
| ATOM | 879 | CA | GLY | A | 118 | 42.866 | 40.903 | 3.807 | 1.00 | 121.24 | | C |
| ANISOU | 879 | CA | GLY | A | 118 | 10516 | 21671 | 13877 | 505 | −250 | −867 | C |
| ATOM | 880 | C | GLY | A | 118 | 42.350 | 41.757 | 4.951 | 1.00 | 123.61 | | C |
| ANISOU | 880 | C | GLY | A | 118 | 10736 | 22003 | 14226 | 632 | −139 | −828 | C |
| ATOM | 881 | O | GLU | A | 119 | 45.190 | 42.116 | 7.819 | 1.00 | 116.71 | | O |
| ANISOU | 881 | O | GLU | A | 119 | 10760 | 19772 | 13814 | 381 | 145 | −502 | O |
| ATOM | 882 | N | GLU | A | 119 | 43.064 | 41.696 | 6.112 | 1.00 | 118.42 | | N |
| ANISOU | 882 | N | GLU | A | 119 | 10336 | 20932 | 13725 | 456 | −27 | −759 | N |
| ATOM | 883 | CA | GLU | A | 119 | 42.826 | 42.460 | 7.348 | 1.00 | 116.99 | | C |
| ANISOU | 883 | CA | GLU | A | 119 | 10163 | 20672 | 13615 | 542 | 94 | −706 | C |
| ATOM | 884 | C | GLU | A | 119 | 44.090 | 42.454 | 8.282 | 1.00 | 117.81 | | C |
| ANISOU | 884 | C | GLU | A | 119 | 10648 | 20220 | 13892 | 425 | 184 | −580 | C |
| ATOM | 885 | CB | GLU | A | 119 | 41.518 | 42.007 | 8.064 | 1.00 | 118.24 | | C |
| ANISOU | 885 | CB | GLU | A | 119 | 10009 | 21210 | 13705 | 306 | 147 | −894 | C |
| ATOM | 886 | CG | GLU | A | 119 | 41.556 | 40.660 | 8.777 | 1.00 | 126.39 | | C |
| ANISOU | 886 | CG | GLU | A | 119 | 11123 | 22118 | 14781 | −218 | 201 | −1026 | C |
| ATOM | 887 | CD | GLU | A | 119 | 41.268 | 39.393 | 7.994 | 1.00 | 140.67 | | C |
| ANISOU | 887 | CD | GLU | A | 119 | 12817 | 24143 | 16489 | −541 | 124 | −1200 | C |
| ATOM | 888 | OE1 | GLU | A | 119 | 40.911 | 38.382 | 8.640 | 1.00 | 133.59 | | O |
| ANISOU | 888 | OE1 | GLU | A | 119 | 11910 | 23266 | 15583 | −966 | 195 | −1346 | O |
| ATOM | 889 | OE2 | GLU | A | 119 | 41.398 | 39.399 | 6.749 | 1.00 | 129.39 | | O |
| ANISOU | 889 | OE2 | GLU | A | 119 | 11326 | 22854 | 14982 | −379 | 1 | −1195 | O |
| ATOM | 890 | O | GLU | A | 120 | 45.627 | 41.551 | 12.567 | 1.00 | 110.66 | | O |
| ANISOU | 890 | O | GLU | A | 120 | 10390 | 18328 | 13326 | −282 | 514 | −536 | O |
| ATOM | 891 | N | GLU | A | 120 | 43.920 | 42.872 | 9.575 | 1.00 | 112.47 | | N |
| ANISOU | 891 | N | GLU | A | 120 | 10000 | 19450 | 13283 | 405 | 300 | −562 | N |
| ATOM | 892 | CA | GLU | A | 120 | 44.942 | 42.909 | 10.641 | 1.00 | 110.78 | | C |
| ANISOU | 892 | CA | GLU | A | 120 | 10097 | 18782 | 13213 | 289 | 390 | −471 | C |
| ATOM | 893 | C | GLU | A | 120 | 44.956 | 41.600 | 11.523 | 1.00 | 110.65 | | C |
| ANISOU | 893 | C | GLU | A | 120 | 10154 | 18660 | 13229 | −168 | 438 | −589 | C |
| ATOM | 894 | CB | GLU | A | 120 | 44.813 | 44.192 | 11.488 | 1.00 | 112.09 | | C |
| ANISOU | 894 | CB | GLU | A | 120 | 10284 | 18889 | 13416 | 546 | 490 | −382 | C |
| ATOM | 895 | N | ARG | A | 121 | 44.194 | 40.555 | 11.071 | 1.00 | 102.64 | | N |
| ANISOU | 895 | N | ARG | A | 121 | 8948 | 17934 | 12116 | −428 | 398 | −755 | N |
| ATOM | 896 | CA | ARG | A | 121 | 44.113 | 39.174 | 11.586 | 1.00 | 99.50 | | C |
| ANISOU | 896 | CA | ARG | A | 121 | 8639 | 17457 | 11709 | −879 | 442 | −884 | C |
| ATOM | 897 | C | ARG | A | 121 | 45.133 | 38.397 | 10.757 | 1.00 | 96.73 | | C |
| ANISOU | 897 | C | ARG | A | 121 | 8539 | 16843 | 11371 | −966 | 360 | −847 | C |
| ATOM | 898 | O | ARG | A | 121 | 45.773 | 37.473 | 11.253 | 1.00 | 95.56 | | O |
| ANISOU | 898 | O | ARG | A | 121 | 8659 | 16389 | 11261 | −1213 | 400 | −852 | O |
| ATOM | 899 | CB | ARG | A | 121 | 42.699 | 38.578 | 11.386 | 1.00 | 98.34 | | C |
| ANISOU | 899 | CB | ARG | A | 121 | 8153 | 17783 | 11431 | −1113 | 446 | −1093 | C |
| ATOM | 900 | CG | ARG | A | 121 | 42.564 | 37.063 | 11.626 | 1.00 | 105.15 | | C |
| ANISOU | 900 | CG | ARG | A | 121 | 9124 | 18579 | 12249 | −1612 | 493 | −1246 | C |
| ATOM | 901 | CD | ARG | A | 121 | 42.860 | 36.647 | 13.061 | 1.00 | 116.77 | | C |
| ANISOU | 901 | CD | ARG | A | 121 | 10853 | 19720 | 13793 | −1846 | 632 | −1229 | C |
| ATOM | 902 | NE | ARG | A | 121 | 42.138 | 37.477 | 14.029 | 1.00 | 129.02 | | N |
| ANISOU | 902 | NE | ARG | A | 121 | 12226 | 21430 | 15366 | −1744 | 729 | −1226 | N |
| ATOM | 903 | CZ | ARG | A | 121 | 42.631 | 37.888 | 15.192 | 1.00 | 144.28 | | C |
| ANISOU | 903 | CZ | ARG | A | 121 | 14361 | 23067 | 17392 | −1687 | 819 | −1123 | C |
| ATOM | 904 | NH1 | ARG | A | 121 | 43.863 | 37.548 | 15.558 | 1.00 | 130.93 | | N |
| ANISOU | 904 | NH1 | ARG | A | 121 | 13051 | 20918 | 15779 | −1739 | 825 | −1022 | N |
| ATOM | 905 | NH2 | ARG | A | 121 | 41.902 | 38.651 | 15.995 | 1.00 | 131.75 | | N |
| ANISOU | 905 | NH2 | ARG | A | 121 | 12594 | 21656 | 15809 | −1577 | 905 | −1130 | N |
| ATOM | 906 | N | ARG | A | 122 | 45.262 | 38.788 | 9.491 | 1.00 | 88.65 | | N |
| ANISOU | 906 | N | ARG | A | 122 | 7436 | 15945 | 10304 | −725 | 246 | −803 | N |
| ATOM | 907 | CA | ARG | A | 122 | 46.305 | 38.209 | 8.609 | 1.00 | 85.82 | | C |
| ANISOU | 907 | CA | ARG | A | 122 | 7279 | 15381 | 9946 | −727 | 159 | −755 | C |
| ATOM | 908 | C | ARG | A | 122 | 47.665 | 38.685 | 9.133 | 1.00 | 83.20 | | C |
| ANISOU | 908 | C | ARG | A | 122 | 7247 | 14627 | 9737 | −561 | 179 | −576 | C |
| ATOM | 909 | O | ARG | A | 122 | 48.660 | 37.954 | 8.959 | 1.00 | 82.86 | | O |
| ANISOU | 909 | O | ARG | A | 122 | 7427 | 14345 | 9712 | −612 | 136 | −535 | O |
| ATOM | 910 | CB | ARG | A | 122 | 46.085 | 38.647 | 7.158 | 1.00 | 85.32 | | C |
| ANISOU | 910 | CB | ARG | A | 122 | 7017 | 15614 | 9786 | −490 | 42 | −760 | C |
| ATOM | 911 | CG | ARG | A | 122 | 46.853 | 37.819 | 6.137 | 1.00 | 93.24 | | C |
| ANISOU | 911 | CG | ARG | A | 122 | 7971 | 16775 | 10680 | −762 | −29 | −915 | C |
| ATOM | 912 | CD | ARG | A | 122 | 46.734 | 38.379 | 4.732 | 1.00 | 93.45 | | C |
| ANISOU | 912 | CD | ARG | A | 122 | 8260 | 16529 | 10719 | −732 | −100 | −840 | C |
| ATOM | 913 | NE | ARG | A | 122 | 46.254 | 37.386 | 3.781 | 1.00 | 101.91 | | N |
| ANISOU | 913 | NE | ARG | A | 122 | 9158 | 17900 | 11665 | −632 | −218 | −902 | N |
| ATOM | 914 | CZ | ARG | A | 122 | 45.720 | 37.674 | 2.600 | 1.00 | 114.72 | | C |
| ANISOU | 914 | CZ | ARG | A | 122 | 10664 | 19667 | 13257 | −251 | −284 | −803 | C |
| ATOM | 915 | NH1 | ARG | A | 122 | 45.596 | 38.934 | 2.219 | 1.00 | 95.87 | | N |
| ANISOU | 915 | NH1 | ARG | A | 122 | 8345 | 17120 | 10961 | 45 | −234 | −636 | N |
| ATOM | 916 | NH2 | ARG | A | 122 | 45.312 | 36.702 | 1.804 | 1.00 | 101.62 | | N |
| ANISOU | 916 | NH2 | ARG | A | 122 | 8844 | 18302 | 11465 | −166 | −395 | −871 | N |
| ATOM | 917 | N | ALA | A | 123 | 47.688 | 39.875 | 9.750 | 1.00 | 74.62 | | N |
| ANISOU | 917 | N | ALA | A | 123 | 6163 | 13464 | 8727 | −366 | 249 | −480 | N |
| ATOM | 918 | CA | ALA | A | 123 | 48.920 | 40.504 | 10.195 | 1.00 | 72.64 | | C |
| ANISOU | 918 | CA | ALA | A | 123 | 6166 | 12845 | 8589 | −226 | 284 | −328 | C |
| ATOM | 919 | CB | ALA | A | 123 | 48.615 | 41.690 | 11.093 | 1.00 | 73.43 | | C |
| ANISOU | 919 | CB | ALA | A | 123 | 6216 | 12948 | 8738 | −44 | 371 | −266 | C |
| ATOM | 920 | C | ALA | A | 123 | 49.735 | 39.478 | 10.953 | 1.00 | 72.70 | | C |
| ANISOU | 920 | C | ALA | A | 123 | 6430 | 12547 | 8645 | −492 | 316 | −343 | C |
| ATOM | 921 | O | ALA | A | 123 | 50.934 | 39.367 | 10.697 | 1.00 | 72.83 | | O |
| ANISOU | 921 | O | ALA | A | 123 | 6656 | 12307 | 8708 | −438 | 284 | −256 | O |
| ATOM | 922 | N | GLU | A | 124 | 49.084 | 38.697 | 11.851 | 1.00 | 65.55 | | N |
| ANISOU | 922 | N | GLU | A | 124 | 5511 | 11683 | 7712 | −776 | 381 | −457 | N |
| ATOM | 923 | CA | GLU | A | 124 | 49.765 | 37.668 | 12.646 | 1.00 | 63.50 | | C |
| ANISOU | 923 | CA | GLU | A | 124 | 5526 | 11131 | 7470 | −1022 | 422 | −475 | C |
| ATOM | 924 | CB | GLU | A | 124 | 48.934 | 37.212 | 13.871 | 1.00 | 65.19 | | C |
| ANISOU | 924 | CB | GLU | A | 124 | 5724 | 11383 | 7662 | −1279 | 533 | −575 | C |
| ATOM | 925 | CG | GLU | A | 124 | 47.563 | 36.628 | 13.555 | 1.00 | 76.38 | | C |
| ANISOU | 925 | CG | GLU | A | 124 | 6900 | 13145 | 8976 | −1500 | 553 | −740 | C |
| ATOM | 926 | CD | GLU | A | 124 | 46.900 | 35.717 | 14.573 | 1.00 | 95.50 | | C |
| ANISOU | 926 | CD | GLU | A | 124 | 9383 | 15557 | 11347 | −1860 | 670 | −863 | C |
| ATOM | 927 | OE1 | GLU | A | 124 | 47.521 | 35.397 | 15.613 | 1.00 | 83.25 | | O |
| ANISOU | 927 | OE1 | GLU | A | 124 | 8091 | 13705 | 9835 | −1941 | 744 | −817 | O |
| ATOM | 928 | OE2 | GLU | A | 124 | 45.746 | 35.306 | 14.312 | 1.00 | 92.82 | | O |
| ANISOU | 928 | OE2 | GLU | A | 124 | 8828 | 15523 | 10916 | −2071 | 692 | −1014 | O |
| ATOM | 929 | C | GLU | A | 124 | 50.226 | 36.473 | 11.808 | 1.00 | 60.45 | | C |
| ANISOU | 929 | C | GLU | A | 124 | 5284 | 10666 | 7020 | −1166 | 353 | −520 | C |
| ATOM | 930 | O | GLU | A | 124 | 51.260 | 35.885 | 12.128 | 1.00 | 60.67 | | O |
| ANISOU | 930 | O | GLU | A | 124 | 5586 | 10396 | 7070 | −1200 | 351 | −468 | O |
| ATOM | 931 | N | ASP | A | 125 | 49.476 | 36.133 | 10.737 | 1.00 | 50.19 | | N |
| ANISOU | 931 | N | ASP | A | 125 | 3802 | 9641 | 5628 | −1229 | 295 | −619 | N |
| ATOM | 932 | CA | ASP | A | 125 | 49.778 | 35.002 | 9.854 | 1.00 | 46.97 | | C |
| ANISOU | 932 | CA | ASP | A | 125 | 3522 | 9184 | 5140 | −1372 | 232 | −679 | C |
| ATOM | 933 | CB | ASP | A | 125 | 48.504 | 34.502 | 9.151 | 1.00 | 48.79 | | C |
| ANISOU | 933 | CB | ASP | A | 125 | 3508 | 9779 | 5251 | −1552 | 204 | −846 | C |
| ATOM | 934 | CG | ASP | A | 125 | 47.482 | 33.846 | 10.089 | 1.00 | 58.84 | | C |
| ANISOU | 934 | CG | ASP | A | 125 | 4705 | 11179 | 6472 | −1891 | 311 | −998 | C |
| ATOM | 935 | OD1 | ASP | A | 125 | 47.872 | 33.452 | 11.237 | 1.00 | 57.56 | | O |
| ANISOU | 935 | OD1 | ASP | A | 125 | 4783 | 10745 | 6341 | −2055 | 409 | −985 | O |
| ATOM | 936 | OD2 | ASP | A | 125 | 46.303 | 33.713 | 9.680 | 1.00 | 64.59 | | O |
| ANISOU | 936 | OD2 | ASP | A | 125 | 5135 | 12290 | 7115 | −1997 | 299 | −1134 | O |
| ATOM | 937 | C | ASP | A | 125 | 50.923 | 35.301 | 8.870 | 1.00 | 43.87 | | C |
| ANISOU | 937 | C | ASP | A | 125 | 3238 | 8654 | 4776 | −1115 | 139 | −556 | C |
| ATOM | 938 | O | ASP | A | 125 | 51.602 | 34.387 | 8.385 | 1.00 | 40.45 | | O |
| ANISOU | 938 | O | ASP | A | 125 | 3012 | 8057 | 4299 | −1192 | 101 | −564 | O |
| ATOM | 939 | N | LEU | A | 126 | 51.157 | 36.590 | 8.625 | 1.00 | 39.18 | | N |
| ANISOU | 939 | N | LEU | A | 126 | 2520 | 8120 | 4247 | −811 | 115 | −442 | N |
| ATOM | 940 | CA | LEU | A | 126 | 52.245 | 37.086 | 7.794 | 1.00 | 38.08 | | C |
| ANISOU | 940 | CA | LEU | A | 126 | 2470 | 7855 | 4142 | −561 | 54 | −315 | C |
| ATOM | 941 | CB | LEU | A | 126 | 51.844 | 38.371 | 7.072 | 1.00 | 38.28 | | C |
| ANISOU | 941 | CB | LEU | A | 126 | 2284 | 8090 | 4169 | −264 | 27 | −246 | C |
| ATOM | 942 | CG | LEU | A | 126 | 51.014 | 38.133 | 5.816 | 1.00 | 44.18 | | C |
| ANISOU | 942 | CG | LEU | A | 126 | 2830 | 9157 | 4798 | −225 | −59 | −326 | C |
| ATOM | 943 | CD1 | LEU | A | 126 | 50.370 | 39.408 | 5.343 | 1.00 | 45.21 | | C |
| ANISOU | 943 | CD1 | LEU | A | 126 | 2761 | 9507 | 4909 | 90 | −73 | −260 | C |
| ATOM | 944 | CD2 | LEU | A | 126 | 51.838 | 37.448 | 4.715 | 1.00 | 45.54 | | C |
| ANISOU | 944 | CD2 | LEU | A | 126 | 3149 | 9231 | 4923 | −233 | −138 | −316 | C |
| ATOM | 945 | C | LEU | A | 126 | 53.406 | 37.339 | 8.692 | 1.00 | 41.06 | | C |
| ANISOU | 945 | C | LEU | A | 126 | 3070 | 7913 | 4618 | −515 | 102 | −211 | C |
| ATOM | 946 | O | LEU | A | 126 | 54.544 | 37.319 | 8.231 | 1.00 | 40.06 | | O |
| ANISOU | 946 | O | LEU | A | 126 | 3092 | 7618 | 4511 | −411 | 65 | −132 | O |
| ATOM | 947 | N | ARG | A | 127 | 53.124 | 37.551 | 10.000 | 1.00 | 37.41 | | N |
| ANISOU | 947 | N | ARG | A | 127 | 2624 | 7384 | 4207 | −600 | 186 | −221 | N |
| ATOM | 948 | CA | ARG | A | 127 | 54.169 | 37.741 | 10.987 | 1.00 | 37.46 | | C |
| ANISOU | 948 | CA | ARG | A | 127 | 2833 | 7109 | 4292 | −579 | 230 | −142 | C |
| ATOM | 949 | CB | ARG | A | 127 | 53.611 | 38.341 | 12.270 | 1.00 | 36.82 | | C |
| ANISOU | 949 | CB | ARG | A | 127 | 2701 | 7028 | 4261 | −614 | 320 | −149 | C |
| ATOM | 950 | CG | ARG | A | 127 | 54.173 | 39.716 | 12.569 | 1.00 | 41.58 | | C |
| ANISOU | 950 | CG | ARG | A | 127 | 3306 | 7538 | 4955 | −393 | 350 | −36 | C |
| ATOM | 951 | CD | ARG | A | 127 | 53.652 | 40.233 | 13.886 | 1.00 | 46.12 | | C |
| ANISOU | 951 | CD | ARG | A | 127 | 3860 | 8097 | 5568 | −438 | 442 | −52 | C |
| ATOM | 952 | NE | ARG | A | 127 | 52.265 | 40.675 | 13.745 | 1.00 | 49.47 | | N |
| ANISOU | 952 | NE | ARG | A | 127 | 4043 | 8798 | 5955 | −403 | 471 | −105 | N |
| ATOM | 953 | CZ | ARG | A | 127 | 51.280 | 40.337 | 14.568 | 1.00 | 55.69 | | C |
| ANISOU | 953 | CZ | ARG | A | 127 | 4749 | 9698 | 6714 | −568 | 536 | −196 | C |
| ATOM | 954 | NH1 | ARG | A | 127 | 51.524 | 39.574 | 15.629 | 1.00 | 36.27 | | N |
| ANISOU | 954 | NH1 | ARG | A | 127 | 2458 | 7067 | 4254 | −783 | 584 | −236 | N |
| ATOM | 955 | NH2 | ARG | A | 127 | 50.045 | 40.775 | 14.350 | 1.00 | 39.97 | | N |
| ANISOU | 955 | NH2 | ARG | A | 127 | 2506 | 8004 | 4677 | −511 | 556 | −250 | N |
| ATOM | 956 | C | ARG | A | 127 | 54.838 | 36.392 | 11.246 | 1.00 | 42.66 | | C |
| ANISOU | 956 | C | ARG | A | 127 | 3739 | 7566 | 4902 | −760 | 221 | −183 | C |
| ATOM | 957 | O | ARG | A | 127 | 56.080 | 36.311 | 11.237 | 1.00 | 43.34 | | O |
| ANISOU | 957 | O | ARG | A | 127 | 3999 | 7456 | 5014 | −671 | 196 | −110 | O |
| ATOM | 958 | N | GLN | A | 128 | 54.023 | 35.353 | 11.416 | 1.00 | 37.99 | | N |
| ANISOU | 958 | N | GLN | A | 128 | 3170 | 7040 | 4226 | −1011 | 244 | −305 | N |
| ATOM | 959 | CA | GLN | A | 128 | 54.531 | 33.977 | 11.644 | 1.00 | 37.58 | | C |
| ANISOU | 959 | CA | GLN | A | 128 | 3396 | 6785 | 4097 | −1189 | 254 | −352 | C |
| ATOM | 960 | CB | GLN | A | 128 | 53.372 | 33.030 | 11.951 | 1.00 | 38.77 | | C |
| ANISOU | 960 | CB | GLN | A | 128 | 3567 | 7001 | 4164 | −1500 | 327 | −490 | C |
| ATOM | 961 | CG | GLN | A | 128 | 53.810 | 31.595 | 12.188 | 1.00 | 55.31 | | C |
| ANISOU | 961 | CG | GLN | A | 128 | 5851 | 8899 | 6265 | −1622 | 426 | −490 | C |
| ATOM | 962 | CD | GLN | A | 128 | 54.375 | 31.392 | 13.570 | 1.00 | 65.01 | | C |
| ANISOU | 962 | CD | GLN | A | 128 | 7455 | 9825 | 7420 | −1681 | 438 | −478 | C |
| ATOM | 963 | OE1 | GLN | A | 128 | 54.495 | 32.327 | 14.356 | 1.00 | 67.00 | | O |
| ANISOU | 963 | OE1 | GLN | A | 128 | 7882 | 9888 | 7689 | −1646 | 482 | −427 | O |
| ATOM | 964 | NE2 | GLN | A | 128 | 54.729 | 30.156 | 13.875 | 1.00 | 40.51 | | N |
| ANISOU | 964 | NE2 | GLN | A | 128 | 4501 | 6673 | 4217 | −1760 | 404 | −527 | N |
| ATOM | 965 | C | GLN | A | 128 | 55.312 | 33.512 | 10.412 | 1.00 | 42.04 | | C |
| ANISOU | 965 | C | GLN | A | 128 | 4061 | 7308 | 4606 | −1121 | 170 | −339 | C |
| ATOM | 966 | O | GLN | A | 128 | 56.303 | 32.788 | 10.582 | 1.00 | 42.66 | | O |
| ANISOU | 966 | O | GLN | A | 128 | 4408 | 7169 | 4632 | −1155 | 168 | −331 | O |
| ATOM | 967 | N | MET | A | 129 | 54.853 | 33.885 | 9.216 | 1.00 | 38.57 | | N |
| ANISOU | 967 | N | MET | A | 129 | 3418 | 7076 | 4162 | −1002 | 104 | −334 | N |
| ATOM | 968 | CA | MET | A | 129 | 55.499 | 33.466 | 7.982 | 1.00 | 39.15 | | C |
| ANISOU | 968 | CA | MET | A | 129 | 3559 | 7134 | 4181 | −908 | 23 | −315 | C |
| ATOM | 969 | CB | MET | A | 129 | 54.896 | 34.215 | 6.787 | 1.00 | 41.48 | | C |
| ANISOU | 969 | CB | MET | A | 129 | 3589 | 7706 | 4467 | −771 | −40 | −314 | C |
| ATOM | 970 | CG | MET | A | 129 | 55.438 | 33.778 | 5.451 | 1.00 | 44.83 | | C |
| ANISOU | 970 | CG | MET | A | 129 | 4075 | 8139 | 4818 | −687 | −122 | −307 | C |
| ATOM | 971 | SD | MET | A | 129 | 55.017 | 34.962 | 4.159 | 1.00 | 49.51 | | S |
| ANISOU | 971 | SD | MET | A | 129 | 4402 | 8991 | 5419 | −409 | −188 | −241 | S |
| ATOM | 972 | CE | MET | A | 129 | 56.291 | 36.160 | 4.380 | 1.00 | 46.59 | | C |
| ANISOU | 972 | CE | MET | A | 129 | 4112 | 8414 | 5177 | −149 | −152 | −59 | C |
| ATOM | 973 | C | MET | A | 129 | 56.976 | 33.782 | 8.098 | 1.00 | 44.47 | | C |
| ANISOU | 973 | C | MET | A | 129 | 4380 | 7601 | 4915 | −704 | 6 | −189 | C |
| ATOM | 974 | O | MET | A | 129 | 57.795 | 32.946 | 7.722 | 1.00 | 44.19 | | O |
| ANISOU | 974 | O | MET | A | 129 | 4538 | 7435 | 4820 | −684 | −29 | −182 | O |
| ATOM | 975 | N | PHE | A | 130 | 57.308 | 34.972 | 8.654 | 1.00 | 41.93 | | N |
| ANISOU | 975 | N | PHE | A | 130 | 3972 | 7256 | 4702 | −559 | 36 | −98 | N |
| ATOM | 976 | CA | PHE | A | 130 | 58.685 | 35.416 | 8.827 | 1.00 | 42.25 | | C |
| ANISOU | 976 | CA | PHE | A | 130 | 4119 | 7135 | 4800 | −391 | 28 | 6 | C |
| ATOM | 977 | CB | PHE | A | 130 | 58.748 | 36.919 | 9.147 | 1.00 | 45.02 | | C |
| ANISOU | 977 | CB | PHE | A | 130 | 4327 | 7519 | 5260 | −243 | 65 | 90 | C |
| ATOM | 978 | CG | PHE | A | 130 | 60.033 | 37.691 | 8.889 | 1.00 | 47.85 | | C |
| ANISOU | 978 | CG | PHE | A | 130 | 4716 | 7788 | 5677 | −65 | 60 | 193 | C |
| ATOM | 979 | CD2 | PHE | A | 130 | 60.040 | 39.084 | 8.900 | 1.00 | 50.91 | | C |
| ANISOU | 979 | CD2 | PHE | A | 130 | 4995 | 8205 | 6144 | 60 | 105 | 264 | C |
| ATOM | 980 | CE2 | PHE | A | 130 | 61.238 | 39.802 | 8.741 | 1.00 | 54.36 | | C |
| ANISOU | 980 | CE2 | PHE | A | 130 | 5470 | 8555 | 6628 | 177 | 121 | 344 | C |
| ATOM | 981 | CZ | PHE | A | 130 | 62.427 | 39.127 | 8.553 | 1.00 | 53.09 | | C |
| ANISOU | 981 | CZ | PHE | A | 130 | 5420 | 8315 | 6437 | 191 | 82 | 352 | C |
| ATOM | 982 | CE1 | PHE | A | 130 | 62.439 | 37.746 | 8.536 | 1.00 | 54.15 | | C |
| ANISOU | 982 | CE1 | PHE | A | 130 | 5664 | 8422 | 6488 | 109 | 29 | 291 | C |
| ATOM | 983 | CD1 | PHE | A | 130 | 61.248 | 37.029 | 8.726 | 1.00 | 52.41 | | C |
| ANISOU | 983 | CD1 | PHE | A | 130 | 5443 | 8251 | 6219 | −29 | 24 | 212 | C |
| ATOM | 984 | C | PHE | A | 130 | 59.326 | 34.586 | 9.911 | 1.00 | 44.08 | | C |
| ANISOU | 984 | C | PHE | A | 130 | 4586 | 7160 | 5001 | −488 | 55 | −13 | C |
| ATOM | 985 | O | PHE | A | 130 | 60.359 | 33.974 | 9.656 | 1.00 | 43.89 | | O |
| ANISOU | 985 | O | PHE | A | 130 | 4732 | 7019 | 4925 | −427 | 20 | 7 | O |
| ATOM | 986 | N | ILE | A | 131 | 58.681 | 34.494 | 11.085 | 1.00 | 39.33 | | N |
| ANISOU | 986 | N | ILE | A | 131 | 4005 | 6524 | 4416 | −626 | 120 | −55 | N |
| ATOM | 987 | CA | ILE | A | 131 | 59.146 | 33.701 | 12.235 | 1.00 | 38.74 | | C |
| ANISOU | 987 | CA | ILE | A | 131 | 4173 | 6254 | 4293 | −720 | 158 | −76 | C |
| ATOM | 988 | CB | ILE | A | 131 | 58.095 | 33.750 | 13.397 | 1.00 | 39.58 | | C |
| ANISOU | 988 | CB | ILE | A | 131 | 4255 | 6376 | 4409 | −904 | 244 | −136 | C |
| ATOM | 989 | CG1 | ILE | A | 131 | 58.143 | 35.084 | 14.177 | 1.00 | 38.51 | | C |
| ANISOU | 989 | CG1 | ILE | A | 131 | 3972 | 6272 | 4388 | −805 | 278 | −78 | C |
| ATOM | 990 | CD1 | ILE | A | 131 | 59.473 | 35.529 | 14.748 | 1.00 | 38.37 | | C |
| ANISOU | 990 | CD1 | ILE | A | 131 | 4047 | 6116 | 4415 | −651 | 258 | 0 | C |
| ATOM | 991 | CG2 | ILE | A | 131 | 58.172 | 32.552 | 14.329 | 1.00 | 38.24 | | C |
| ANISOU | 991 | CG2 | ILE | A | 131 | 4373 | 6017 | 4140 | −1059 | 296 | −186 | C |
| ATOM | 992 | C | ILE | A | 131 | 59.518 | 32.261 | 11.831 | 1.00 | 47.48 | | C |
| ANISOU | 992 | C | ILE | A | 131 | 5528 | 7243 | 5269 | −784 | 133 | −121 | C |
| ATOM | 993 | O | ILE | A | 131 | 60.558 | 31.755 | 12.265 | 1.00 | 48.95 | | O |
| ANISOU | 993 | O | ILE | A | 131 | 5924 | 7266 | 5408 | −697 | 121 | −87 | O |
| ATOM | 994 | N | ALA | A | 132 | 58.672 | 31.630 | 10.989 | 1.00 | 44.96 | | N |
| ANISOU | 994 | N | ALA | A | 132 | 5188 | 7017 | 4878 | −925 | 126 | −202 | N |
| ATOM | 995 | CA | ALA | A | 132 | 58.801 | 30.261 | 10.516 | 1.00 | 45.54 | | C |
| ANISOU | 995 | CA | ALA | A | 132 | 5515 | 6976 | 4813 | −1018 | 117 | −262 | C |
| ATOM | 996 | CB | ALA | A | 132 | 57.507 | 29.818 | 9.865 | 1.00 | 46.14 | | C |
| ANISOU | 996 | CB | ALA | A | 132 | 5511 | 7197 | 4825 | −1252 | 135 | −383 | C |
| ATOM | 997 | C | ALA | A | 132 | 59.958 | 30.022 | 9.569 | 1.00 | 53.12 | | C |
| ANISOU | 997 | C | ALA | A | 132 | 6559 | 7886 | 5737 | −810 | 41 | −203 | C |
| ATOM | 998 | O | ALA | A | 132 | 60.541 | 28.937 | 9.609 | 1.00 | 53.18 | | O |
| ANISOU | 998 | O | ALA | A | 132 | 6857 | 7720 | 5630 | −803 | 43 | −217 | O |
| ATOM | 999 | N | MET | A | 133 | 60.268 | 30.995 | 8.693 | 1.00 | 52.74 | | N |
| ANISOU | 999 | N | MET | A | 133 | 6281 | 7988 | 5771 | −638 | −17 | −139 | N |
| ATOM | 1000 | CA | MET | A | 133 | 61.335 | 30.853 | 7.689 | 1.00 | 54.35 | | C |
| ANISOU | 1000 | CA | MET | A | 133 | 6543 | 8167 | 5940 | −442 | −81 | −82 | C |
| ATOM | 1001 | CB | MET | A | 133 | 60.940 | 31.473 | 6.328 | 1.00 | 56.40 | | C |
| ANISOU | 1001 | CB | MET | A | 133 | 6573 | 8625 | 6234 | −355 | −132 | −63 | C |
| ATOM | 1002 | CG | MET | A | 133 | 60.797 | 32.982 | 6.341 | 1.00 | 59.79 | | C |
| ANISOU | 1002 | CG | MET | A | 133 | 6756 | 9165 | 6795 | −231 | −123 | 18 | C |
| ATOM | 1003 | SD | MET | A | 133 | 61.587 | 33.765 | 4.928 | 1.00 | 64.30 | | S |
| ANISOU | 1003 | SD | MET | A | 133 | 7160 | 9887 | 7383 | −31 | −174 | 88 | S |
| ATOM | 1004 | CE | MET | A | 133 | 63.340 | 33.605 | 5.374 | 1.00 | 61.11 | | C |
| ANISOU | 1004 | CE | MET | A | 133 | 6909 | 9345 | 6966 | 154 | −190 | 170 | C |
| ATOM | 1005 | C | MET | A | 133 | 62.706 | 31.371 | 8.153 | 1.00 | 62.85 | | C |
| ANISOU | 1005 | C | MET | A | 133 | 7663 | 9154 | 7063 | −241 | −90 | 12 | C |
| ATOM | 1006 | O | MET | A | 133 | 63.733 | 30.877 | 7.677 | 1.00 | 64.14 | | O |
| ANISOU | 1006 | O | MET | A | 133 | 7971 | 9246 | 7155 | −106 | −125 | 37 | O |
| ATOM | 1007 | N | ALA | A | 134 | 62.725 | 32.363 | 9.049 | 1.00 | 60.87 | | N |
| ANISOU | 1007 | N | ALA | A | 134 | 7270 | 8931 | 6926 | −216 | −61 | 56 | N |
| ATOM | 1008 | CA | ALA | A | 134 | 63.968 | 32.920 | 9.557 | 1.00 | 61.81 | | C |
| ANISOU | 1008 | CA | ALA | A | 134 | 7391 | 9002 | 7090 | −57 | −68 | 126 | C |
| ATOM | 1009 | CB | ALA | A | 134 | 63.856 | 34.436 | 9.689 | 1.00 | 62.55 | | C |
| ANISOU | 1009 | CB | ALA | A | 134 | 7248 | 9196 | 7323 | −11 | −43 | 181 | C |
| ATOM | 1010 | C | ALA | A | 134 | 64.321 | 32.271 | 10.893 | 1.00 | 69.28 | | C |
| ANISOU | 1010 | C | ALA | A | 134 | 8543 | 9801 | 7980 | −94 | −43 | 104 | C |
| ATOM | 1011 | O | ALA | A | 134 | 63.909 | 32.755 | 11.964 | 1.00 | 70.07 | | O |
| ANISOU | 1011 | O | ALA | A | 134 | 8605 | 9881 | 8138 | −179 | 2 | 95 | O |
| ATOM | 1012 | N | GLU | A | 135 | 65.055 | 31.136 | 10.824 | 1.00 | 66.92 | | N |
| ANISOU | 1012 | N | GLU | A | 135 | 8484 | 9393 | 7550 | −17 | −69 | 93 | N |
| ATOM | 1013 | CA | GLU | A | 135 | 65.498 | 30.408 | 12.023 | 1.00 | 67.01 | | C |
| ANISOU | 1013 | CA | GLU | A | 135 | 8736 | 9254 | 7469 | −3 | −49 | 78 | C |
| ATOM | 1014 | CB | GLU | A | 135 | 66.013 | 29.000 | 11.669 | 1.00 | 69.21 | | C |
| ANISOU | 1014 | CB | GLU | A | 135 | 9323 | 9403 | 7569 | 76 | −66 | 58 | C |
| ATOM | 1015 | CG | GLU | A | 135 | 65.174 | 27.855 | 12.233 | 1.00 | 85.80 | | C |
| ANISOU | 1015 | CG | GLU | A | 135 | 11719 | 11328 | 9552 | −110 | 1 | −10 | C |
| ATOM | 1016 | CD | GLU | A | 135 | 65.843 | 26.486 | 12.320 | 1.00 | 119.80 | | C |
| ANISOU | 1016 | CD | GLU | A | 135 | 16417 | 15446 | 13656 | 7 | 5 | −19 | C |
| ATOM | 1017 | OE1 | GLU | A | 135 | 66.802 | 26.222 | 11.555 | 1.00 | 122.77 | | O |
| ANISOU | 1017 | OE1 | GLU | A | 135 | 16830 | 15849 | 13969 | 225 | −53 | 13 | O |
| ATOM | 1018 | OE2 | GLU | A | 135 | 65.394 | 25.670 | 13.157 | 1.00 | 115.18 | | O |
| ANISOU | 1018 | OE2 | GLU | A | 135 | 16119 | 14682 | 12963 | −115 | 77 | −59 | O |
| ATOM | 1019 | C | GLU | A | 135 | 66.579 | 31.238 | 12.740 | 1.00 | 67.20 | | C |
| ANISOU | 1019 | C | GLU | A | 135 | 8652 | 9327 | 7552 | 148 | −72 | 129 | C |
| ATOM | 1020 | O | GLU | A | 135 | 66.632 | 31.254 | 13.980 | 1.00 | 67.97 | | O |
| ANISOU | 1020 | O | GLU | A | 135 | 8826 | 9360 | 7641 | 119 | −46 | 119 | O |
| ATOM | 1021 | N | ASP | A | 136 | 67.417 | 31.953 | 11.956 | 1.00 | 59.26 | | N |
| ANISOU | 1021 | N | ASP | A | 136 | 7466 | 8446 | 6604 | 291 | −113 | 175 | N |
| ATOM | 1022 | CA | ASP | A | 136 | 68.415 | 32.844 | 12.520 | 1.00 | 56.85 | | C |
| ANISOU | 1022 | CA | ASP | A | 136 | 7026 | 8218 | 6358 | 391 | −126 | 204 | C |
| ATOM | 1023 | CB | ASP | A | 136 | 69.692 | 32.950 | 11.671 | 1.00 | 58.73 | | C |
| ANISOU | 1023 | CB | ASP | A | 136 | 7181 | 8566 | 6567 | 578 | −173 | 236 | C |
| ATOM | 1024 | CG | ASP | A | 136 | 70.859 | 33.562 | 12.433 | 1.00 | 70.28 | | C |
| ANISOU | 1024 | CG | ASP | A | 136 | 8535 | 10122 | 8047 | 670 | −189 | 239 | C |
| ATOM | 1025 | OD1 | ASP | A | 136 | 70.722 | 33.790 | 13.656 | 1.00 | 72.62 | | O |
| ANISOU | 1025 | OD1 | ASP | A | 136 | 8856 | 10378 | 8359 | 610 | −175 | 217 | O |
| ATOM | 1026 | OD2 | ASP | A | 136 | 71.907 | 33.799 | 11.816 | 1.00 | 76.47 | | O |
| ANISOU | 1026 | OD2 | ASP | A | 136 | 9204 | 11031 | 8819 | 794 | −214 | 256 | O |
| ATOM | 1027 | C | ASP | A | 136 | 67.750 | 34.180 | 12.693 | 1.00 | 55.62 | | C |
| ANISOU | 1027 | C | ASP | A | 136 | 6651 | 8123 | 6360 | 277 | −80 | 220 | C |
| ATOM | 1028 | O | ASP | A | 136 | 67.675 | 34.985 | 11.761 | 1.00 | 56.27 | | O |
| ANISOU | 1028 | O | ASP | A | 136 | 6558 | 8296 | 6524 | 292 | −72 | 254 | O |
| ATOM | 1029 | N | VAL | A | 137 | 67.223 | 34.381 | 13.894 | 1.00 | 47.61 | | N |
| ANISOU | 1029 | N | VAL | A | 137 | 5672 | 7047 | 5372 | 173 | −42 | 196 | N |
| ATOM | 1030 | CA | VAL | A | 137 | 66.516 | 35.567 | 14.377 | 1.00 | 45.35 | | C |
| ANISOU | 1030 | CA | VAL | A | 137 | 5227 | 6790 | 5213 | 70 | 13 | 203 | C |
| ATOM | 1031 | CB | VAL | A | 137 | 66.332 | 35.437 | 15.909 | 1.00 | 48.03 | | C |
| ANISOU | 1031 | CB | VAL | A | 137 | 5677 | 7043 | 5530 | 0 | 42 | 170 | C |
| ATOM | 1032 | CG1 | VAL | A | 137 | 67.669 | 35.312 | 16.637 | 1.00 | 47.87 | | C |
| ANISOU | 1032 | CG1 | VAL | A | 137 | 5725 | 7023 | 5442 | 124 | −3 | 165 | C |
| ATOM | 1033 | CG2 | VAL | A | 137 | 65.495 | 36.565 | 16.475 | 1.00 | 47.55 | | C |
| ANISOU | 1033 | CG2 | VAL | A | 137 | 5480 | 7000 | 5585 | −101 | 107 | 171 | C |
| ATOM | 1034 | C | VAL | A | 137 | 67.195 | 36.895 | 13.928 | 1.00 | 46.59 | | C |
| ANISOU | 1034 | C | VAL | A | 137 | 5183 | 7046 | 5474 | 135 | 22 | 246 | C |
| ATOM | 1035 | O | VAL | A | 137 | 66.498 | 37.861 | 13.630 | 1.00 | 46.56 | | O |
| ANISOU | 1035 | O | VAL | A | 137 | 5050 | 7073 | 5565 | 84 | 71 | 267 | O |
| ATOM | 1036 | N | ARG | A | 138 | 68.537 | 36.894 | 13.790 | 1.00 | 40.63 | | N |
| ANISOU | 1036 | N | ARG | A | 138 | 4410 | 6346 | 4683 | 251 | −17 | 256 | N |
| ATOM | 1037 | CA | ARG | A | 138 | 69.361 | 38.014 | 13.321 | 1.00 | 39.04 | | C |
| ANISOU | 1037 | CA | ARG | A | 138 | 4041 | 6238 | 4556 | 291 | 3 | 285 | C |
| ATOM | 1038 | CB | ARG | A | 138 | 70.851 | 37.621 | 13.284 | 1.00 | 36.88 | | C |
| ANISOU | 1038 | CB | ARG | A | 138 | 3760 | 6051 | 4201 | 413 | −49 | 271 | C |
| ATOM | 1039 | CG | ARG | A | 138 | 71.397 | 37.236 | 14.654 | 1.00 | 40.99 | | C |
| ANISOU | 1039 | CG | ARG | A | 138 | 4361 | 6563 | 4651 | 434 | −84 | 221 | C |
| ATOM | 1040 | CD | ARG | A | 138 | 72.862 | 36.839 | 14.632 | 1.00 | 41.95 | | C |
| ANISOU | 1040 | CD | ARG | A | 138 | 4450 | 6817 | 4671 | 584 | −145 | 197 | C |
| ATOM | 1041 | NE | ARG | A | 138 | 73.073 | 35.539 | 14.000 | 1.00 | 46.77 | | N |
| ANISOU | 1041 | NE | ARG | A | 138 | 5201 | 7414 | 5156 | 731 | −198 | 208 | N |
| ATOM | 1042 | CZ | ARG | A | 138 | 74.195 | 34.837 | 14.098 | 1.00 | 58.83 | | C |
| ANISOU | 1042 | CZ | ARG | A | 138 | 6755 | 9046 | 6553 | 911 | −259 | 187 | C |
| ATOM | 1043 | NH1 | ARG | A | 138 | 75.233 | 35.319 | 14.775 | 1.00 | 38.69 | | N |
| ANISOU | 1043 | NH1 | ARG | A | 138 | 4072 | 6650 | 3980 | 956 | −284 | 145 | N |
| ATOM | 1044 | NH2 | ARG | A | 138 | 74.306 | 33.664 | 13.485 | 1.00 | 47.52 | | N |
| ANISOU | 1044 | NH2 | ARG | A | 138 | 5485 | 7573 | 4996 | 1053 | −296 | 199 | N |
| ATOM | 1045 | C | ARG | A | 138 | 68.903 | 38.558 | 11.949 | 1.00 | 40.54 | | C |
| ANISOU | 1045 | C | ARG | A | 138 | 4131 | 6470 | 4803 | 302 | 33 | 335 | C |
| ATOM | 1046 | O | ARG | A | 138 | 69.155 | 39.726 | 11.662 | 1.00 | 41.23 | | O |
| ANISOU | 1046 | O | ARG | A | 138 | 4104 | 6592 | 4969 | 291 | 88 | 366 | O |
| ATOM | 1047 | N | ILE | A | 139 | 68.231 | 37.726 | 11.115 | 1.00 | 33.39 | | N |
| ANISOU | 1047 | N | ILE | A | 139 | 3284 | 5556 | 3845 | 321 | 2 | 339 | N |
| ATOM | 1048 | CA | ILE | A | 139 | 67.683 | 38.152 | 9.822 | 1.00 | 31.58 | | C |
| ANISOU | 1048 | CA | ILE | A | 139 | 2968 | 5381 | 3649 | 346 | 19 | 380 | C |
| ATOM | 1049 | CB | ILE | A | 139 | 67.126 | 36.950 | 9.016 | 1.00 | 33.51 | | C |
| ANISOU | 1049 | CB | ILE | A | 139 | 3304 | 5625 | 3803 | 357 | −33 | 358 | C |
| ATOM | 1050 | CG1 | ILE | A | 139 | 68.231 | 36.119 | 8.402 | 1.00 | 33.39 | | C |
| ANISOU | 1050 | CG1 | ILE | A | 139 | 3370 | 5625 | 3693 | 475 | −84 | 360 | C |
| ATOM | 1051 | CD1 | ILE | A | 139 | 67.910 | 34.676 | 8.338 | 1.00 | 40.72 | | C |
| ANISOU | 1051 | CD1 | ILE | A | 139 | 4483 | 6485 | 4503 | 462 | −128 | 314 | C |
| ATOM | 1052 | CG2 | ILE | A | 139 | 66.137 | 37.398 | 7.946 | 1.00 | 33.60 | | C |
| ANISOU | 1052 | CG2 | ILE | A | 139 | 3217 | 5707 | 3841 | 356 | −22 | 382 | C |
| ATOM | 1053 | C | ILE | A | 139 | 66.575 | 39.182 | 10.117 | 1.00 | 35.52 | | C |
| ANISOU | 1053 | C | ILE | A | 139 | 3383 | 5873 | 4238 | 272 | 82 | 394 | C |
| ATOM | 1054 | O | ILE | A | 139 | 66.550 | 40.246 | 9.481 | 1.00 | 35.85 | | O |
| ANISOU | 1054 | O | ILE | A | 139 | 3333 | 5952 | 4337 | 315 | 130 | 445 | O |
| ATOM | 1055 | N | ILE | A | 140 | 65.666 | 38.857 | 11.087 | 1.00 | 29.52 | | N |
| ANISOU | 1055 | N | ILE | A | 140 | 2673 | 5065 | 3479 | 171 | 90 | 349 | N |
| ATOM | 1056 | CA | ILE | A | 140 | 64.560 | 39.736 | 11.476 | 1.00 | 28.38 | | C |
| ANISOU | 1056 | CA | ILE | A | 140 | 2450 | 4930 | 3402 | 115 | 150 | 353 | C |
| ATOM | 1057 | CB | ILE | A | 140 | 63.555 | 39.068 | 12.462 | 1.00 | 30.62 | | C |
| ANISOU | 1057 | CB | ILE | A | 140 | 2795 | 5180 | 3659 | −9 | 157 | 290 | C |
| ATOM | 1058 | CG1 | ILE | A | 140 | 63.075 | 37.683 | 11.949 | 1.00 | 30.48 | | C |
| ANISOU | 1058 | CG1 | ILE | A | 140 | 2862 | 5175 | 3546 | −64 | 107 | 243 | C |
| ATOM | 1059 | CD1 | ILE | A | 140 | 62.615 | 36.639 | 13.066 | 1.00 | 27.42 | | C |
| ANISOU | 1059 | CD1 | ILE | A | 140 | 2626 | 4700 | 3094 | −208 | 121 | 173 | C |
| ATOM | 1060 | CG2 | ILE | A | 140 | 62.379 | 40.010 | 12.805 | 1.00 | 29.53 | | C |
| ANISOU | 1060 | CG2 | ILE | A | 140 | 2550 | 5087 | 3583 | −43 | 222 | 291 | C |
| ATOM | 1061 | C | ILE | A | 140 | 65.112 | 41.069 | 11.999 | 1.00 | 33.70 | | C |
| ANISOU | 1061 | C | ILE | A | 140 | 3075 | 5571 | 4157 | 131 | 216 | 386 | C |
| ATOM | 1062 | O | ILE | A | 140 | 64.720 | 42.111 | 11.485 | 1.00 | 34.04 | | O |
| ANISOU | 1062 | O | ILE | A | 140 | 3047 | 5639 | 4249 | 179 | 271 | 431 | O |
| ATOM | 1063 | N | ILE | A | 141 | 66.064 | 41.034 | 12.957 | 1.00 | 30.62 | | N |
| ANISOU | 1063 | N | ILE | A | 141 | 2738 | 5130 | 3765 | 100 | 211 | 361 | N |
| ATOM | 1064 | CA | ILE | A | 141 | 66.679 | 42.235 | 13.524 | 1.00 | 30.70 | | C |
| ANISOU | 1064 | CA | ILE | A | 141 | 2714 | 5111 | 3839 | 80 | 274 | 370 | C |
| ATOM | 1065 | CB | ILE | A | 141 | 67.740 | 41.884 | 14.565 | 1.00 | 33.56 | | C |
| ANISOU | 1065 | CB | ILE | A | 141 | 3126 | 5460 | 4166 | 48 | 239 | 320 | C |
| ATOM | 1066 | CG1 | ILE | A | 141 | 67.111 | 41.136 | 15.758 | 1.00 | 34.20 | | C |
| ANISOU | 1066 | CG1 | ILE | A | 141 | 3305 | 5481 | 4210 | −11 | 218 | 274 | C |
| ATOM | 1067 | CD1 | ILE | A | 141 | 68.005 | 40.054 | 16.359 | 1.00 | 42.06 | | C |
| ANISOU | 1067 | CD1 | ILE | A | 141 | 4399 | 6475 | 5106 | 21 | 145 | 232 | C |
| ATOM | 1068 | CG2 | ILE | A | 141 | 68.458 | 43.144 | 15.028 | 1.00 | 33.87 | | C |
| ANISOU | 1068 | CG2 | ILE | A | 141 | 3118 | 5490 | 4262 | 0 | 304 | 313 | C |
| ATOM | 1069 | C | ILE | A | 141 | 67.242 | 43.156 | 12.440 | 1.00 | 38.06 | | C |
| ANISOU | 1069 | C | ILE | A | 141 | 3580 | 6074 | 4806 | 138 | 322 | 426 | C |
| ATOM | 1070 | O | ILE | A | 141 | 67.048 | 44.369 | 12.530 | 1.00 | 40.26 | | O |
| ANISOU | 1070 | O | ILE | A | 141 | 3846 | 6310 | 5142 | 123 | 410 | 453 | O |
| ATOM | 1071 | N | VAL | A | 142 | 67.912 | 42.594 | 11.414 | 1.00 | 33.26 | | N |
| ANISOU | 1071 | N | VAL | A | 142 | 2954 | 5530 | 4152 | 208 | 277 | 445 | N |
| ATOM | 1072 | CA | VAL | A | 142 | 68.457 | 43.403 | 10.327 | 1.00 | 32.33 | | C |
| ANISOU | 1072 | CA | VAL | A | 142 | 2788 | 5441 | 4054 | 259 | 333 | 500 | C |
| ATOM | 1073 | CB | VAL | A | 142 | 69.539 | 42.697 | 9.462 | 1.00 | 34.47 | | C |
| ANISOU | 1073 | CB | VAL | A | 142 | 3037 | 5796 | 4265 | 325 | 284 | 504 | C |
| ATOM | 1074 | CG1 | VAL | A | 142 | 70.083 | 43.630 | 8.390 | 1.00 | 33.22 | | C |
| ANISOU | 1074 | CG1 | VAL | A | 142 | 2836 | 5659 | 4126 | 356 | 367 | 562 | C |
| ATOM | 1075 | CG2 | VAL | A | 142 | 70.684 | 42.223 | 10.342 | 1.00 | 34.30 | | C |
| ANISOU | 1075 | CG2 | VAL | A | 142 | 3009 | 5815 | 4208 | 295 | 240 | 442 | C |
| ATOM | 1076 | C | VAL | A | 142 | 67.294 | 44.023 | 9.521 | 1.00 | 37.44 | | C |
| ANISOU | 1076 | C | VAL | A | 142 | 3425 | 6080 | 4722 | 322 | 380 | 559 | C |
| ATOM | 1077 | O | VAL | A | 142 | 67.354 | 45.229 | 9.261 | 1.00 | 39.62 | | O |
| ANISOU | 1077 | O | VAL | A | 142 | 3707 | 6312 | 5036 | 336 | 477 | 605 | O |
| ATOM | 1078 | N | LYS | A | 143 | 66.230 | 43.261 | 9.261 | 1.00 | 30.43 | | N |
| ANISOU | 1078 | N | LYS | A | 143 | 2529 | 5234 | 3797 | 356 | 319 | 550 | N |
| ATOM | 1079 | CA | LYS | A | 143 | 65.061 | 43.830 | 8.540 | 1.00 | 28.36 | | C |
| ANISOU | 1079 | CA | LYS | A | 143 | 2229 | 5010 | 3535 | 434 | 349 | 591 | C |
| ATOM | 1080 | CB | LYS | A | 143 | 64.022 | 42.743 | 8.257 | 1.00 | 29.99 | | C |
| ANISOU | 1080 | CB | LYS | A | 143 | 2404 | 5304 | 3688 | 428 | 269 | 548 | C |
| ATOM | 1081 | CG | LYS | A | 143 | 63.333 | 42.846 | 6.904 | 1.00 | 41.78 | | C |
| ANISOU | 1081 | CG | LYS | A | 143 | 3840 | 6906 | 5128 | 547 | 245 | 584 | C |
| ATOM | 1082 | CD | LYS | A | 143 | 62.918 | 44.257 | 6.554 | 1.00 | 46.87 | | C |
| ANISOU | 1082 | CD | LYS | A | 143 | 4450 | 7574 | 5786 | 678 | 322 | 654 | C |
| ATOM | 1083 | CE | LYS | A | 143 | 62.880 | 44.516 | 5.064 | 1.00 | 51.18 | | C |
| ANISOU | 1083 | CE | LYS | A | 143 | 4989 | 8186 | 6272 | 820 | 312 | 714 | C |
| ATOM | 1084 | NZ | LYS | A | 143 | 62.733 | 45.960 | 4.767 | 1.00 | 53.53 | | N |
| ANISOU | 1084 | NZ | LYS | A | 143 | 5295 | 8489 | 6556 | 980 | 396 | 792 | N |
| ATOM | 1085 | C | LYS | A | 143 | 64.476 | 44.961 | 9.392 | 1.00 | 30.90 | | C |
| ANISOU | 1085 | C | LYS | A | 143 | 2560 | 5268 | 3914 | 416 | 438 | 601 | C |
| ATOM | 1086 | O | LYS | A | 143 | 64.077 | 45.983 | 8.818 | 1.00 | 30.71 | | O |
| ANISOU | 1086 | O | LYS | A | 143 | 2541 | 5231 | 3895 | 512 | 513 | 661 | O |
| ATOM | 1087 | N | LEU | A | 144 | 64.422 | 44.763 | 10.712 | 1.00 | 26.28 | | N |
| ANISOU | 1087 | N | LEU | A | 144 | 1999 | 4630 | 3357 | 309 | 438 | 546 | N |
| ATOM | 1088 | CA | LEU | A | 144 | 63.941 | 45.752 | 11.656 | 1.00 | 26.04 | | C |
| ANISOU | 1088 | CA | LEU | A | 144 | 1993 | 4529 | 3374 | 286 | 523 | 545 | C |
| ATOM | 1089 | CB | LEU | A | 144 | 63.817 | 45.151 | 13.050 | 1.00 | 25.40 | | C |
| ANISOU | 1089 | CB | LEU | A | 144 | 1937 | 4414 | 3301 | 167 | 497 | 474 | C |
| ATOM | 1090 | CG | LEU | A | 144 | 62.768 | 44.060 | 13.179 | 1.00 | 29.97 | | C |
| ANISOU | 1090 | CG | LEU | A | 144 | 2482 | 5067 | 3838 | 134 | 435 | 429 | C |
| ATOM | 1091 | CD1 | LEU | A | 144 | 62.699 | 43.532 | 14.559 | 1.00 | 29.07 | | C |
| ANISOU | 1091 | CD1 | LEU | A | 144 | 2427 | 4898 | 3722 | 19 | 429 | 368 | C |
| ATOM | 1092 | CD2 | LEU | A | 144 | 61.409 | 44.560 | 12.802 | 1.00 | 35.32 | | C |
| ANISOU | 1092 | CD2 | LEU | A | 144 | 3079 | 5829 | 4512 | 207 | 472 | 445 | C |
| ATOM | 1093 | C | LEU | A | 144 | 64.792 | 47.019 | 11.654 | 1.00 | 34.86 | | C |
| ANISOU | 1093 | C | LEU | A | 144 | 3170 | 5547 | 4528 | 285 | 625 | 584 | C |
| ATOM | 1094 | O | LEU | A | 144 | 64.234 | 48.117 | 11.588 | 1.00 | 35.75 | | O |
| ANISOU | 1094 | O | LEU | A | 144 | 3324 | 5605 | 4654 | 351 | 720 | 626 | O |
| ATOM | 1095 | N | ALA | A | 145 | 66.133 | 46.877 | 11.634 | 1.00 | 33.35 | | N |
| ANISOU | 1095 | N | ALA | A | 145 | 2990 | 5342 | 4339 | 216 | 615 | 567 | N |
| ATOM | 1096 | CA | ALA | A | 145 | 67.062 | 48.016 | 11.609 | 1.00 | 33.87 | | C |
| ANISOU | 1096 | CA | ALA | A | 145 | 3108 | 5331 | 4431 | 165 | 721 | 584 | C |
| ATOM | 1097 | CB | ALA | A | 145 | 68.487 | 47.531 | 11.766 | 1.00 | 34.52 | | C |
| ANISOU | 1097 | CB | ALA | A | 145 | 3149 | 5467 | 4501 | 74 | 676 | 533 | C |
| ATOM | 1098 | C | ALA | A | 145 | 66.913 | 48.794 | 10.317 | 1.00 | 40.41 | | C |
| ANISOU | 1098 | C | ALA | A | 145 | 3972 | 6138 | 5242 | 275 | 800 | 670 | C |
| ATOM | 1099 | O | ALA | A | 145 | 66.863 | 50.018 | 10.327 | 1.00 | 41.33 | | O |
| ANISOU | 1099 | O | ALA | A | 145 | 4186 | 6145 | 5371 | 280 | 927 | 707 | O |
| ATOM | 1100 | N | ASP | A | 146 | 66.786 | 48.075 | 9.217 | 1.00 | 37.64 | | N |
| ANISOU | 1100 | N | ASP | A | 146 | 3568 | 5884 | 4851 | 372 | 730 | 703 | N |
| ATOM | 1101 | CA | ASP | A | 146 | 66.586 | 48.635 | 7.897 | 1.00 | 38.51 | | C |
| ANISOU | 1101 | CA | ASP | A | 146 | 3712 | 5997 | 4924 | 504 | 785 | 787 | C |
| ATOM | 1102 | CB | ASP | A | 146 | 66.618 | 47.488 | 6.861 | 1.00 | 41.18 | | C |
| ANISOU | 1102 | CB | ASP | A | 146 | 3974 | 6463 | 5209 | 578 | 673 | 793 | C |
| ATOM | 1103 | CG | ASP | A | 146 | 66.015 | 47.801 | 5.509 | 1.00 | 55.23 | | C |
| ANISOU | 1103 | CG | ASP | A | 146 | 5770 | 8287 | 6930 | 749 | 690 | 871 | C |
| ATOM | 1104 | OD1 | ASP | A | 146 | 66.754 | 48.319 | 4.633 | 1.00 | 56.63 | | O |
| ANISOU | 1104 | OD1 | ASP | A | 146 | 5999 | 8436 | 7082 | 786 | 761 | 927 | O |
| ATOM | 1105 | OD2 | ASP | A | 146 | 64.809 | 47.515 | 5.318 | 1.00 | 62.61 | | O |
| ANISOU | 1105 | OD2 | ASP | A | 146 | 6658 | 9298 | 7832 | 846 | 634 | 871 | O |
| ATOM | 1106 | C | ASP | A | 146 | 65.253 | 49.405 | 7.842 | 1.00 | 42.31 | | C |
| ANISOU | 1106 | C | ASP | A | 146 | 4240 | 6448 | 5391 | 637 | 839 | 835 | C |
| ATOM | 1107 | O | ASP | A | 146 | 65.227 | 50.532 | 7.321 | 1.00 | 41.66 | | O |
| ANISOU | 1107 | O | ASP | A | 146 | 4266 | 6277 | 5287 | 723 | 959 | 905 | O |
| ATOM | 1108 | N | ARG | A | 147 | 64.155 | 48.792 | 8.386 | 1.00 | 37.92 | | N |
| ANISOU | 1108 | N | ARG | A | 147 | 3608 | 5968 | 4833 | 658 | 761 | 793 | N |
| ATOM | 1109 | CA | ARG | A | 147 | 62.817 | 49.385 | 8.365 | 1.00 | 36.47 | | C |
| ANISOU | 1109 | CA | ARG | A | 147 | 3424 | 5812 | 4622 | 802 | 797 | 823 | C |
| ATOM | 1110 | CB | ARG | A | 147 | 61.713 | 48.424 | 8.838 | 1.00 | 33.24 | | C |
| ANISOU | 1110 | CB | ARG | A | 147 | 2884 | 5547 | 4200 | 791 | 693 | 758 | C |
| ATOM | 1111 | CG | ARG | A | 147 | 60.294 | 49.013 | 8.760 | 1.00 | 38.51 | | C |
| ANISOU | 1111 | CG | ARG | A | 147 | 3508 | 6301 | 4822 | 957 | 725 | 777 | C |
| ATOM | 1112 | CD | ARG | A | 147 | 59.964 | 49.683 | 7.418 | 1.00 | 47.18 | | C |
| ANISOU | 1112 | CD | ARG | A | 147 | 4626 | 7459 | 5841 | 1188 | 753 | 862 | C |
| ATOM | 1113 | NE | ARG | A | 147 | 59.607 | 48.719 | 6.378 | 1.00 | 49.55 | | N |
| ANISOU | 1113 | NE | ARG | A | 147 | 4799 | 7949 | 6079 | 1234 | 633 | 842 | N |
| ATOM | 1114 | CZ | ARG | A | 147 | 58.360 | 48.368 | 6.077 | 1.00 | 65.88 | | C |
| ANISOU | 1114 | CZ | ARG | A | 147 | 6722 | 10230 | 8081 | 1330 | 566 | 806 | C |
| ATOM | 1115 | NH1 | ARG | A | 147 | 57.334 | 48.926 | 6.707 | 1.00 | 51.97 | | N |
| ANISOU | 1115 | NH1 | ARG | A | 147 | 4912 | 8530 | 6304 | 1414 | 609 | 792 | N |
| ATOM | 1116 | NH2 | ARG | A | 147 | 58.127 | 47.474 | 5.125 | 1.00 | 55.89 | | N |
| ANISOU | 1116 | NH2 | ARG | A | 147 | 5352 | 9131 | 6754 | 1340 | 458 | 774 | N |
| ATOM | 1117 | C | ARG | A | 147 | 62.810 | 50.646 | 9.152 | 1.00 | 39.81 | | C |
| ANISOU | 1117 | C | ARG | A | 147 | 3971 | 6080 | 5075 | 793 | 930 | 840 | C |
| ATOM | 1118 | O | ARG | A | 147 | 62.361 | 51.672 | 8.632 | 1.00 | 39.15 | | O |
| ANISOU | 1118 | O | ARG | A | 147 | 3982 | 5946 | 4948 | 955 | 1025 | 911 | O |
| ATOM | 1119 | N | LEU | A | 148 | 63.357 | 50.589 | 10.384 | 1.00 | 36.07 | | N |
| ANISOU | 1119 | N | LEU | A | 148 | 3520 | 5525 | 4661 | 614 | 941 | 775 | N |
| ATOM | 1120 | CA | LEU | A | 148 | 63.454 | 51.751 | 11.248 | 1.00 | 35.33 | | C |
| ANISOU | 1120 | CA | LEU | A | 148 | 3560 | 5271 | 4593 | 572 | 1069 | 774 | C |
| ATOM | 1121 | CB | LEU | A | 148 | 64.141 | 51.375 | 12.551 | 1.00 | 34.60 | | C |
| ANISOU | 1121 | CB | LEU | A | 148 | 3456 | 5137 | 4553 | 366 | 1040 | 684 | C |
| ATOM | 1122 | CG | LEU | A | 148 | 64.675 | 52.523 | 13.390 | 1.00 | 38.80 | | C |
| ANISOU | 1122 | CG | LEU | A | 148 | 4135 | 5498 | 5109 | 261 | 1170 | 663 | C |
| ATOM | 1123 | CD1 | LEU | A | 148 | 63.551 | 53.363 | 13.989 | 1.00 | 38.77 | | C |
| ANISOU | 1123 | CD1 | LEU | A | 148 | 4222 | 5416 | 5093 | 369 | 1256 | 681 | C |
| ATOM | 1124 | CD2 | LEU | A | 148 | 65.620 | 52.019 | 14.440 | 1.00 | 40.43 | | C |
| ANISOU | 1124 | CD2 | LEU | A | 148 | 4301 | 5711 | 5349 | 60 | 1115 | 568 | C |
| ATOM | 1125 | C | LEU | A | 148 | 64.186 | 52.898 | 10.519 | 1.00 | 42.22 | | C |
| ANISOU | 1125 | C | LEU | A | 148 | 4593 | 6004 | 5445 | 598 | 1208 | 841 | C |
| ATOM | 1126 | O | LEU | A | 148 | 63.653 | 54.017 | 10.481 | 1.00 | 42.74 | | O |
| ANISOU | 1126 | O | LEU | A | 148 | 4806 | 5955 | 5480 | 711 | 1331 | 893 | O |
| ATOM | 1127 | N | HIS | A | 149 | 65.361 | 52.608 | 9.893 | 1.00 | 39.12 | | N |
| ANISOU | 1127 | N | HIS | A | 149 | 4183 | 5626 | 5055 | 508 | 1196 | 844 | N |
| ATOM | 1128 | CA | HIS | A | 149 | 66.093 | 53.644 | 9.182 | 1.00 | 39.41 | | C |
| ANISOU | 1128 | CA | HIS | A | 149 | 4376 | 5534 | 5066 | 500 | 1344 | 902 | C |
| ATOM | 1129 | CB | HIS | A | 149 | 67.497 | 53.233 | 8.750 | 1.00 | 39.73 | | C |
| ANISOU | 1129 | CB | HIS | A | 149 | 4357 | 5620 | 5118 | 346 | 1331 | 873 | C |
| ATOM | 1130 | CG | HIS | A | 149 | 68.204 | 54.337 | 8.016 | 1.00 | 42.98 | | C |
| ANISOU | 1130 | CG | HIS | A | 149 | 4936 | 5900 | 5495 | 313 | 1505 | 929 | C |
| ATOM | 1131 | ND1 | HIS | A | 149 | 68.504 | 54.232 | 6.673 | 1.00 | 44.58 | | N |
| ANISOU | 1131 | ND1 | HIS | A | 149 | 5146 | 6148 | 5645 | 413 | 1519 | 1001 | N |
| ATOM | 1132 | CE1 | HIS | A | 149 | 69.070 | 55.380 | 6.341 | 1.00 | 43.99 | | C |
| ANISOU | 1132 | CE1 | HIS | A | 149 | 5262 | 5913 | 5541 | 342 | 1708 | 1039 | C |
| ATOM | 1133 | NE2 | HIS | A | 149 | 69.110 | 56.220 | 7.371 | 1.00 | 44.25 | | N |
| ANISOU | 1133 | NE2 | HIS | A | 149 | 5418 | 5795 | 5600 | 202 | 1815 | 992 | N |
| ATOM | 1134 | CD2 | HIS | A | 149 | 68.534 | 55.580 | 8.437 | 1.00 | 44.40 | | C |
| ANISOU | 1134 | CD2 | HIS | A | 149 | 5308 | 5890 | 5671 | 196 | 1682 | 924 | C |
| ATOM | 1135 | C | HIS | A | 149 | 65.312 | 54.211 | 8.019 | 1.00 | 46.63 | | C |
| ANISOU | 1135 | C | HIS | A | 149 | 5383 | 6432 | 5903 | 746 | 1402 | 1011 | C |
| ATOM | 1136 | O | HIS | A | 149 | 65.304 | 55.436 | 7.860 | 1.00 | 48.78 | | O |
| ANISOU | 1136 | O | HIS | A | 149 | 5866 | 6532 | 6137 | 799 | 1566 | 1068 | O |
| ATOM | 1137 | N | ASN | A | 150 | 64.638 | 53.357 | 7.220 | 1.00 | 42.06 | | N |
| ANISOU | 1137 | N | ASN | A | 150 | 4668 | 6027 | 5287 | 902 | 1276 | 1036 | N |
| ATOM | 1138 | CA | ASN | A | 150 | 63.837 | 53.866 | 6.105 | 1.00 | 41.46 | | C |
| ANISOU | 1138 | CA | ASN | A | 150 | 4663 | 5974 | 5118 | 1164 | 1313 | 1133 | C |
| ATOM | 1139 | CB | ASN | A | 150 | 63.167 | 52.738 | 5.318 | 1.00 | 44.24 | | C |
| ANISOU | 1139 | CB | ASN | A | 150 | 4823 | 6556 | 5430 | 1284 | 1146 | 1126 | C |
| ATOM | 1140 | CG | ASN | A | 150 | 64.082 | 51.914 | 4.450 | 1.00 | 77.46 | | C |
| ANISOU | 1140 | CG | ASN | A | 150 | 8966 | 10836 | 9630 | 1224 | 1079 | 1127 | C |
| ATOM | 1141 | OD1 | ASN | A | 150 | 65.216 | 52.302 | 4.129 | 1.00 | 78.12 | | O |
| ANISOU | 1141 | OD1 | ASN | A | 150 | 9143 | 10815 | 9724 | 1126 | 1169 | 1150 | O |
| ATOM | 1142 | ND2 | ASN | A | 150 | 63.588 | 50.751 | 4.029 | 1.00 | 69.89 | | N |
| ANISOU | 1142 | ND2 | ASN | A | 150 | 7848 | 10064 | 8643 | 1277 | 927 | 1095 | N |
| ATOM | 1143 | C | ASN | A | 150 | 62.795 | 54.856 | 6.626 | 1.00 | 43.53 | | C |
| ANISOU | 1143 | C | ASN | A | 150 | 5046 | 6152 | 5342 | 1323 | 1405 | 1166 | C |
| ATOM | 1144 | O | ASN | A | 150 | 62.724 | 55.974 | 6.126 | 1.00 | 42.22 | | O |
| ANISOU | 1144 | O | ASN | A | 150 | 5093 | 5846 | 5104 | 1467 | 1549 | 1251 | O |
| ATOM | 1145 | N | LEU | A | 151 | 62.069 | 54.477 | 7.696 | 1.00 | 40.33 | | N |
| ANISOU | 1145 | N | LEU | A | 151 | 4530 | 5815 | 4978 | 1290 | 1338 | 1097 | N |
| ATOM | 1146 | CA | LEU | A | 151 | 61.037 | 55.305 | 8.325 | 1.00 | 40.29 | | C |
| ANISOU | 1146 | CA | LEU | A | 151 | 4608 | 5761 | 4938 | 1441 | 1414 | 1112 | C |
| ATOM | 1147 | CB | LEU | A | 151 | 60.388 | 54.586 | 9.521 | 1.00 | 39.70 | | C |
| ANISOU | 1147 | CB | LEU | A | 151 | 4362 | 5801 | 4922 | 1343 | 1318 | 1016 | C |
| ATOM | 1148 | CG | LEU | A | 151 | 59.355 | 53.523 | 9.195 | 1.00 | 42.70 | | C |
| ANISOU | 1148 | CG | LEU | A | 151 | 4502 | 6452 | 5270 | 1441 | 1169 | 982 | C |
| ATOM | 1149 | CD2 | LEU | A | 151 | 58.020 | 54.142 | 8.854 | 1.00 | 43.65 | | C |
| ANISOU | 1149 | CD2 | LEU | A | 151 | 4616 | 6678 | 5291 | 1737 | 1205 | 1027 | C |
| ATOM | 1150 | CD1 | LEU | A | 151 | 59.202 | 52.575 | 10.336 | 1.00 | 42.37 | | C |
| ANISOU | 1150 | CD1 | LEU | A | 151 | 4318 | 6480 | 5300 | 1239 | 1082 | 879 | C |
| ATOM | 1151 | C | LEU | A | 151 | 61.584 | 56.644 | 8.761 | 1.00 | 47.05 | | C |
| ANISOU | 1151 | C | LEU | A | 151 | 5744 | 6341 | 5791 | 1394 | 1608 | 1144 | C |
| ATOM | 1152 | O | LEU | A | 151 | 60.914 | 57.662 | 8.575 | 1.00 | 46.55 | | O |
| ANISOU | 1152 | O | LEU | A | 151 | 5856 | 6186 | 5646 | 1615 | 1722 | 1210 | O |
| ATOM | 1153 | N | ARG | A | 152 | 62.815 | 56.649 | 9.310 | 1.00 | 45.90 | | N |
| ANISOU | 1153 | N | ARG | A | 152 | 5648 | 6071 | 5721 | 1111 | 1649 | 1090 | N |
| ATOM | 1154 | CA | ARG | A | 152 | 63.486 | 57.869 | 9.750 | 1.00 | 47.06 | | C |
| ANISOU | 1154 | CA | ARG | A | 152 | 6060 | 5955 | 5864 | 997 | 1840 | 1095 | C |
| ATOM | 1155 | CB | ARG | A | 152 | 64.841 | 57.541 | 10.363 | 1.00 | 45.25 | | C |
| ANISOU | 1155 | CB | ARG | A | 152 | 5781 | 5692 | 5719 | 659 | 1830 | 1000 | C |
| ATOM | 1156 | CG | ARG | A | 152 | 64.729 | 56.980 | 11.762 | 1.00 | 53.45 | | C |
| ANISOU | 1156 | CG | ARG | A | 152 | 6687 | 6791 | 6829 | 515 | 1735 | 894 | C |
| ATOM | 1157 | CD | ARG | A | 152 | 66.057 | 56.425 | 12.197 | 1.00 | 59.21 | | C |
| ANISOU | 1157 | CD | ARG | A | 152 | 7307 | 7570 | 7620 | 235 | 1677 | 802 | C |
| ATOM | 1158 | NE | ARG | A | 152 | 66.074 | 56.068 | 13.612 | 1.00 | 58.13 | | N |
| ANISOU | 1158 | NE | ARG | A | 152 | 7101 | 7452 | 7532 | 93 | 1616 | 701 | N |
| ATOM | 1159 | CZ | ARG | A | 152 | 67.146 | 55.598 | 14.235 | 1.00 | 70.95 | | C |
| ANISOU | 1159 | CZ | ARG | A | 152 | 8635 | 9131 | 9193 | −131 | 1560 | 606 | C |
| ATOM | 1160 | NH1 | ARG | A | 152 | 68.284 | 55.425 | 13.573 | 1.00 | 65.18 | | N |
| ANISOU | 1160 | NH1 | ARG | A | 152 | 7850 | 8455 | 8459 | −244 | 1560 | 594 | N |
| ATOM | 1161 | NH2 | ARG | A | 152 | 67.094 | 55.300 | 15.525 | 1.00 | 55.39 | | N |
| ANISOU | 1161 | NH2 | ARG | A | 152 | 6621 | 7177 | 7249 | −232 | 1505 | 520 | N |
| ATOM | 1162 | C | ARG | A | 152 | 63.645 | 58.854 | 8.595 | 1.00 | 55.63 | | C |
| ANISOU | 1162 | C | ARG | A | 152 | 7392 | 6892 | 6853 | 1146 | 1996 | 1206 | C |
| ATOM | 1163 | O | ARG | A | 152 | 63.328 | 60.038 | 8.745 | 1.00 | 55.99 | | O |
| ANISOU | 1163 | O | ARG | A | 152 | 7711 | 6732 | 6832 | 1253 | 2165 | 1255 | O |
| ATOM | 1164 | N | THR | A | 153 | 64.045 | 58.329 | 7.426 | 1.00 | 54.37 | | N |
| ANISOU | 1164 | N | THR | A | 153 | 7151 | 6837 | 6671 | 1181 | 1941 | 1249 | N |
| ATOM | 1165 | CA | THR | A | 153 | 64.274 | 59.062 | 6.182 | 1.00 | 55.15 | | C |
| ANISOU | 1165 | CA | THR | A | 153 | 7460 | 6826 | 6670 | 1321 | 2072 | 1359 | C |
| ATOM | 1166 | CB | THR | A | 153 | 65.540 | 58.490 | 5.516 | 1.00 | 64.68 | | C |
| ANISOU | 1166 | CB | THR | A | 153 | 8572 | 8091 | 7914 | 1117 | 2045 | 1339 | C |
| ATOM | 1167 | OG1 | THR | A | 153 | 65.317 | 57.114 | 5.170 | 1.00 | 62.38 | | O |
| ANISOU | 1167 | OG1 | THR | A | 153 | 7980 | 8066 | 7655 | 1161 | 1827 | 1308 | O |
| ATOM | 1168 | CG2 | THR | A | 153 | 66.777 | 58.616 | 6.397 | 1.00 | 65.63 | | C |
| ANISOU | 1168 | CG2 | THR | A | 153 | 8697 | 8120 | 8120 | 754 | 2109 | 1239 | C |
| ATOM | 1169 | C | THR | A | 153 | 63.063 | 58.959 | 5.220 | 1.00 | 61.60 | | C |
| ANISOU | 1169 | C | THR | A | 153 | 8244 | 7784 | 7376 | 1701 | 2004 | 1450 | C |
| ATOM | 1170 | O | THR | A | 153 | 63.261 | 58.955 | 4.004 | 1.00 | 62.25 | | O |
| ANISOU | 1170 | O | THR | A | 153 | 8379 | 7891 | 7383 | 1824 | 2021 | 1527 | O |
| ATOM | 1171 | N | LEU | A | 154 | 61.823 | 58.861 | 5.748 | 1.00 | 58.88 | | N |
| ANISOU | 1171 | N | LEU | A | 154 | 7801 | 7557 | 7014 | 1887 | 1926 | 1435 | N |
| ATOM | 1172 | CA | LEU | A | 154 | 60.621 | 58.713 | 4.914 | 1.00 | 58.84 | | C |
| ANISOU | 1172 | CA | LEU | A | 154 | 7711 | 7750 | 6896 | 2243 | 1843 | 1497 | C |
| ATOM | 1173 | CB | LEU | A | 154 | 59.525 | 57.935 | 5.673 | 1.00 | 57.72 | | C |
| ANISOU | 1173 | CB | LEU | A | 154 | 7291 | 7850 | 6790 | 2288 | 1685 | 1414 | C |
| ATOM | 1174 | CG | LEU | A | 154 | 58.572 | 57.068 | 4.858 | 1.00 | 60.16 | | C |
| ANISOU | 1174 | CG | LEU | A | 154 | 7345 | 8484 | 7031 | 2491 | 1513 | 1411 | C |
| ATOM | 1175 | CD1 | LEU | A | 154 | 59.259 | 55.859 | 4.310 | 1.00 | 59.60 | | C |
| ANISOU | 1175 | CD1 | LEU | A | 154 | 7087 | 8535 | 7021 | 2298 | 1376 | 1365 | C |
| ATOM | 1176 | CD2 | LEU | A | 154 | 57.462 | 56.589 | 5.709 | 1.00 | 61.85 | | C |
| ANISOU | 1176 | CD2 | LEU | A | 154 | 7338 | 8902 | 7261 | 2529 | 1415 | 1330 | C |
| ATOM | 1177 | C | LEU | A | 154 | 60.088 | 60.048 | 4.349 | 1.00 | 68.63 | | C |
| ANISOU | 1177 | C | LEU | A | 154 | 9267 | 8832 | 7976 | 2573 | 2013 | 1618 | C |
| ATOM | 1178 | O | LEU | A | 154 | 59.197 | 60.036 | 3.499 | 1.00 | 67.75 | | O |
| ANISOU | 1178 | O | LEU | A | 154 | 9111 | 8890 | 7741 | 2902 | 1955 | 1680 | O |
| ATOM | 1179 | N | GLU | A | 155 | 60.663 | 61.190 | 4.773 | 1.00 | 70.88 | | N |
| ANISOU | 1179 | N | GLU | A | 155 | 9888 | 8793 | 8248 | 2489 | 2227 | 1649 | N |
| ATOM | 1180 | CA | GLU | A | 155 | 60.274 | 62.532 | 4.299 | 1.00 | 73.50 | | C |
| ANISOU | 1180 | CA | GLU | A | 155 | 10605 | 8907 | 8415 | 2787 | 2426 | 1769 | C |
| ATOM | 1181 | CB | GLU | A | 155 | 60.965 | 63.659 | 5.115 | 1.00 | 75.35 | | C |
| ANISOU | 1181 | CB | GLU | A | 155 | 11198 | 8766 | 8665 | 2586 | 2660 | 1760 | C |
| ATOM | 1182 | CG | GLU | A | 155 | 62.494 | 63.643 | 5.138 | 1.00 | 89.83 | | C |
| ANISOU | 1182 | CG | GLU | A | 155 | 13104 | 10434 | 10595 | 2163 | 2749 | 1713 | C |
| ATOM | 1183 | CD | GLU | A | 155 | 63.180 | 62.618 | 6.033 | 1.00 | 121.13 | | C |
| ANISOU | 1183 | CD | GLU | A | 155 | 16743 | 14540 | 14741 | 1784 | 2602 | 1571 | C |
| ATOM | 1184 | OE1 | GLU | A | 155 | 62.478 | 61.923 | 6.805 | 1.00 | 120.18 | | O |
| ANISOU | 1184 | OE1 | GLU | A | 155 | 16364 | 14611 | 14687 | 1811 | 2439 | 1503 | O |
| ATOM | 1185 | OE2 | GLU | A | 155 | 64.426 | 62.508 | 5.958 | 1.00 | 116.39 | | O |
| ANISOU | 1185 | OE2 | GLU | A | 155 | 16152 | 13867 | 14205 | 1464 | 2655 | 1525 | O |
| ATOM | 1186 | C | GLU | A | 155 | 60.485 | 62.725 | 2.793 | 1.00 | 82.05 | | C |
| ANISOU | 1186 | C | GLU | A | 155 | 11826 | 9979 | 9372 | 2978 | 2472 | 1883 | C |
| ATOM | 1187 | O | GLU | A | 155 | 59.891 | 63.632 | 2.205 | 1.00 | 81.97 | | O |
| ANISOU | 1187 | O | GLU | A | 155 | 12090 | 9868 | 9188 | 3329 | 2590 | 1995 | O |
| ATOM | 1188 | N | HIS | A | 156 | 61.312 | 61.842 | 2.182 | 1.00 | 82.24 | | N |
| ANISOU | 1188 | N | HIS | A | 156 | 11664 | 10114 | 9471 | 2765 | 2377 | 1855 | N |
| ATOM | 1189 | CA | HIS | A | 156 | 61.699 | 61.819 | 0.761 | 1.00 | 83.64 | | C |
| ANISOU | 1189 | CA | HIS | A | 156 | 11928 | 10303 | 9550 | 2881 | 2404 | 1947 | C |
| ATOM | 1190 | CB | HIS | A | 156 | 63.206 | 61.512 | 0.636 | 1.00 | 85.36 | | C |
| ANISOU | 1190 | CB | HIS | A | 156 | 12135 | 10424 | 9875 | 2484 | 2456 | 1904 | C |
| ATOM | 1191 | CG | HIS | A | 156 | 64.061 | 62.298 | 1.583 | 1.00 | 89.67 | | C |
| ANISOU | 1191 | CG | HIS | A | 156 | 12894 | 10685 | 10492 | 2169 | 2644 | 1856 | C |
| ATOM | 1192 | ND1 | HIS | A | 156 | 64.287 | 63.655 | 1.401 | 1.00 | 91.69 | | N |
| ANISOU | 1192 | ND1 | HIS | A | 156 | 13594 | 10609 | 10637 | 2215 | 2912 | 1938 | N |
| ATOM | 1193 | CE1 | HIS | A | 156 | 65.058 | 64.028 | 2.411 | 1.00 | 91.44 | | C |
| ANISOU | 1193 | CE1 | HIS | A | 156 | 13639 | 10402 | 10701 | 1863 | 3019 | 1847 | C |
| ATOM | 1194 | NE2 | HIS | A | 156 | 65.333 | 63.003 | 3.223 | 1.00 | 91.80 | | N |
| ANISOU | 1194 | NE2 | HIS | A | 156 | 13316 | 10656 | 10906 | 1625 | 2831 | 1720 | N |
| ATOM | 1195 | CD2 | HIS | A | 156 | 64.701 | 61.893 | 2.705 | 1.00 | 91.88 | | C |
| ANISOU | 1195 | CD2 | HIS | A | 156 | 13009 | 10974 | 10927 | 1818 | 2598 | 1728 | C |
| ATOM | 1196 | C | HIS | A | 156 | 60.869 | 60.816 | −0.067 | 1.00 | 86.79 | | C |
| ANISOU | 1196 | C | HIS | A | 156 | 12020 | 11061 | 9894 | 3124 | 2176 | 1950 | C |
| ATOM | 1197 | O | HIS | A | 156 | 61.287 | 60.387 | −1.155 | 1.00 | 86.05 | | O |
| ANISOU | 1197 | O | HIS | A | 156 | 11886 | 11049 | 9761 | 3142 | 2133 | 1988 | O |
| ATOM | 1198 | N | MET | A | 157 | 59.676 | 60.484 | 0.442 | 1.00 | 83.21 | | N |
| ANISOU | 1198 | N | MET | A | 157 | 11359 | 10828 | 9429 | 3306 | 2040 | 1905 | N |
| ATOM | 1199 | CA | MET | A | 157 | 58.764 | 59.544 | −0.189 | 1.00 | 83.30 | | C |
| ANISOU | 1199 | CA | MET | A | 157 | 11057 | 11209 | 9386 | 3507 | 1822 | 1879 | C |
| ATOM | 1200 | CB | MET | A | 157 | 58.518 | 58.333 | 0.727 | 1.00 | 86.29 | | C |
| ANISOU | 1200 | CB | MET | A | 157 | 11058 | 11809 | 9919 | 3258 | 1632 | 1733 | C |
| ATOM | 1201 | CG | MET | A | 157 | 59.742 | 57.480 | 0.958 | 1.00 | 90.96 | | C |
| ANISOU | 1201 | CG | MET | A | 157 | 11546 | 12349 | 10668 | 2859 | 1586 | 1663 | C |
| ATOM | 1202 | SD | MET | A | 157 | 60.599 | 57.005 | −0.559 | 1.00 | 96.28 | | S |
| ANISOU | 1202 | SD | MET | A | 157 | 12235 | 13060 | 11289 | 2855 | 1558 | 1720 | S |
| ATOM | 1203 | CE | MET | A | 157 | 59.372 | 55.873 | −1.284 | 1.00 | 93.15 | | C |
| ANISOU | 1203 | CE | MET | A | 157 | 11510 | 13076 | 10806 | 3075 | 1311 | 1673 | C |
| ATOM | 1204 | C | MET | A | 157 | 57.429 | 60.174 | −0.522 | 1.00 | 85.60 | | C |
| ANISOU | 1204 | C | MET | A | 157 | 11402 | 11631 | 9491 | 3968 | 1822 | 1943 | C |
| ATOM | 1205 | O | MET | A | 157 | 56.948 | 61.001 | 0.261 | 1.00 | 84.13 | | O |
| ANISOU | 1205 | O | MET | A | 157 | 11374 | 11319 | 9273 | 4088 | 1932 | 1959 | O |
| ATOM | 1206 | N | PRO | A | 158 | 56.768 | 59.727 | −1.627 | 1.00 | 82.56 | | N |
| ANISOU | 1206 | N | PRO | A | 158 | 10866 | 11530 | 8973 | 4240 | 1688 | 1968 | N |
| ATOM | 1207 | CA | PRO | A | 158 | 55.417 | 60.247 | −1.941 | 1.00 | 82.76 | | C |
| ANISOU | 1207 | CA | PRO | A | 158 | 10888 | 11756 | 8800 | 4713 | 1662 | 2015 | C |
| ATOM | 1208 | CB | PRO | A | 158 | 54.986 | 59.406 | −3.154 | 1.00 | 84.42 | | C |
| ANISOU | 1208 | CB | PRO | A | 158 | 10859 | 12311 | 8906 | 4873 | 1475 | 2003 | C |
| ATOM | 1209 | CG | PRO | A | 158 | 55.950 | 58.243 | −3.212 | 1.00 | 88.24 | | C |
| ANISOU | 1209 | CG | PRO | A | 158 | 11148 | 12811 | 9569 | 4444 | 1371 | 1917 | C |
| ATOM | 1210 | CD | PRO | A | 158 | 57.217 | 58.745 | −2.640 | 1.00 | 83.72 | | C |
| ANISOU | 1210 | CD | PRO | A | 158 | 10837 | 11843 | 9130 | 4142 | 1553 | 1949 | C |
| ATOM | 1211 | C | PRO | A | 158 | 54.460 | 60.053 | −0.749 | 1.00 | 87.04 | | C |
| ANISOU | 1211 | C | PRO | A | 158 | 11193 | 12481 | 9396 | 4715 | 1586 | 1913 | C |
| ATOM | 1212 | O | PRO | A | 158 | 54.550 | 58.992 | −0.131 | 1.00 | 87.76 | | O |
| ANISOU | 1212 | O | PRO | A | 158 | 10978 | 12719 | 9647 | 4396 | 1450 | 1788 | O |
| ATOM | 1213 | N | PRO | A | 159 | 53.585 | 61.022 | −0.358 | 1.00 | 82.83 | | N |
| ANISOU | 1213 | N | PRO | A | 159 | 10804 | 11933 | 8735 | 5054 | 1680 | 1960 | N |
| ATOM | 1214 | CA | PRO | A | 159 | 52.747 | 60.821 | 0.852 | 1.00 | 82.52 | | C |
| ANISOU | 1214 | CA | PRO | A | 159 | 10533 | 12061 | 8760 | 5020 | 1623 | 1856 | C |
| ATOM | 1215 | CB | PRO | A | 159 | 51.999 | 62.149 | 1.006 | 1.00 | 84.37 | | C |
| ANISOU | 1215 | CB | PRO | A | 159 | 11049 | 12200 | 8808 | 5466 | 1776 | 1950 | C |
| ATOM | 1216 | CG | PRO | A | 159 | 52.811 | 63.134 | 0.240 | 1.00 | 88.94 | | C |
| ANISOU | 1216 | CG | PRO | A | 159 | 12108 | 12393 | 9291 | 5575 | 1974 | 2099 | C |
| ATOM | 1217 | CD | PRO | A | 159 | 53.369 | 62.364 | −0.926 | 1.00 | 84.50 | | C |
| ANISOU | 1217 | CD | PRO | A | 159 | 11430 | 11942 | 8736 | 5472 | 1863 | 2111 | C |
| ATOM | 1218 | C | PRO | A | 159 | 51.796 | 59.620 | 0.818 | 1.00 | 85.25 | | C |
| ANISOU | 1218 | C | PRO | A | 159 | 10381 | 12895 | 9115 | 5003 | 1384 | 1724 | C |
| ATOM | 1219 | O | PRO | A | 159 | 51.160 | 59.319 | 1.832 | 1.00 | 83.35 | | O |
| ANISOU | 1219 | O | PRO | A | 159 | 9928 | 12803 | 8939 | 4919 | 1339 | 1627 | O |
| ATOM | 1220 | N | GLU | A | 160 | 51.741 | 58.915 | −0.337 | 1.00 | 82.41 | | N |
| ANISOU | 1220 | N | GLU | A | 160 | 9846 | 12775 | 8690 | 5050 | 1240 | 1714 | N |
| ATOM | 1221 | CA | GLU | A | 160 | 50.942 | 57.707 | −0.541 | 1.00 | 82.20 | | C |
| ANISOU | 1221 | CA | GLU | A | 160 | 9368 | 13202 | 8662 | 4983 | 1017 | 1577 | C |
| ATOM | 1222 | CB | GLU | A | 160 | 50.450 | 57.597 | −2.001 | 1.00 | 84.21 | | C |
| ANISOU | 1222 | CB | GLU | A | 160 | 9535 | 13747 | 8715 | 5304 | 905 | 1610 | C |
| ATOM | 1223 | CG | GLU | A | 160 | 49.042 | 57.019 | −2.117 | 1.00 | 101.21 | | C |
| ANISOU | 1223 | CG | GLU | A | 160 | 11268 | 16436 | 10751 | 5474 | 728 | 1486 | C |
| ATOM | 1224 | CD | GLU | A | 160 | 47.917 | 57.762 | −1.409 | 1.00 | 130.00 | | C |
| ANISOU | 1224 | CD | GLU | A | 160 | 14859 | 20243 | 14291 | 5786 | 780 | 1478 | C |
| ATOM | 1225 | OE1 | GLU | A | 160 | 47.231 | 58.572 | −2.075 | 1.00 | 135.23 | | O |
| ANISOU | 1225 | OE1 | GLU | A | 160 | 15604 | 21056 | 14720 | 6283 | 799 | 1558 | O |
| ATOM | 1226 | OE2 | GLU | A | 160 | 47.712 | 57.527 | −0.193 | 1.00 | 119.52 | | O |
| ANISOU | 1226 | OE2 | GLU | A | 160 | 13412 | 18901 | 13100 | 5554 | 802 | 1393 | O |
| ATOM | 1227 | C | GLU | A | 160 | 51.710 | 56.458 | −0.070 | 1.00 | 82.16 | | C |
| ANISOU | 1227 | C | GLU | A | 160 | 9188 | 13152 | 8878 | 4465 | 928 | 1468 | C |
| ATOM | 1228 | O | GLU | A | 160 | 51.229 | 55.768 | 0.835 | 1.00 | 81.86 | | O |
| ANISOU | 1228 | O | GLU | A | 160 | 8899 | 13271 | 8935 | 4257 | 854 | 1345 | O |
| ATOM | 1229 | N | LYS | A | 161 | 52.925 | 56.214 | −0.635 | 1.00 | 75.32 | | N |
| ANISOU | 1229 | N | LYS | A | 161 | 8478 | 12058 | 8082 | 4268 | 951 | 1516 | N |
| ATOM | 1230 | CA | LYS | A | 161 | 53.849 | 55.138 | −0.251 | 1.00 | 73.95 | | C |
| ANISOU | 1230 | CA | LYS | A | 161 | 8208 | 11791 | 8097 | 3820 | 890 | 1436 | C |
| ATOM | 1231 | CB | LYS | A | 161 | 55.104 | 55.171 | −1.154 | 1.00 | 76.39 | | C |
| ANISOU | 1231 | CB | LYS | A | 161 | 8732 | 11875 | 8418 | 3745 | 945 | 1522 | C |
| ATOM | 1232 | CG | LYS | A | 161 | 56.174 | 54.101 | −0.867 | 1.00 | 94.94 | | C |
| ANISOU | 1232 | CG | LYS | A | 161 | 11000 | 14136 | 10936 | 3331 | 885 | 1450 | C |
| ATOM | 1233 | CD | LYS | A | 161 | 55.716 | 52.652 | −1.155 | 1.00 | 106.63 | | C |
| ANISOU | 1233 | CD | LYS | A | 161 | 12165 | 15929 | 12421 | 3204 | 679 | 1328 | C |
| ATOM | 1234 | CE | LYS | A | 161 | 56.761 | 51.610 | −0.819 | 1.00 | 112.38 | | C |
| ANISOU | 1234 | CE | LYS | A | 161 | 12860 | 16546 | 13294 | 2840 | 633 | 1267 | C |
| ATOM | 1235 | NZ | LYS | A | 161 | 56.191 | 50.235 | −0.768 | 1.00 | 116.05 | | N |
| ANISOU | 1235 | NZ | LYS | A | 161 | 13053 | 17257 | 13782 | 2662 | 465 | 1127 | N |
| ATOM | 1236 | C | LYS | A | 161 | 54.230 | 55.290 | 1.248 | 1.00 | 76.07 | | C |
| ANISOU | 1236 | C | LYS | A | 161 | 8536 | 11834 | 8532 | 3550 | 979 | 1394 | C |
| ATOM | 1237 | O | LYS | A | 161 | 54.386 | 54.295 | 1.960 | 1.00 | 76.01 | | O |
| ANISOU | 1237 | O | LYS | A | 161 | 8340 | 11885 | 8655 | 3237 | 892 | 1283 | O |
| ATOM | 1238 | N | GLN | A | 162 | 54.311 | 56.550 | 1.719 | 1.00 | 70.25 | | N |
| ANISOU | 1238 | N | GLN | A | 162 | 8078 | 10844 | 7771 | 3691 | 1156 | 1479 | N |
| ATOM | 1239 | CA | GLN | A | 162 | 54.582 | 56.965 | 3.095 | 1.00 | 68.65 | | C |
| ANISOU | 1239 | CA | GLN | A | 162 | 7982 | 10416 | 7687 | 3505 | 1264 | 1452 | C |
| ATOM | 1240 | CB | GLN | A | 162 | 54.417 | 58.495 | 3.181 | 1.00 | 69.40 | | C |
| ANISOU | 1240 | CB | GLN | A | 162 | 8421 | 10275 | 7674 | 3793 | 1461 | 1565 | C |
| ATOM | 1241 | CG | GLN | A | 162 | 55.533 | 59.256 | 3.876 | 1.00 | 69.66 | | C |
| ANISOU | 1241 | CG | GLN | A | 162 | 8771 | 9892 | 7805 | 3564 | 1643 | 1599 | C |
| ATOM | 1242 | CD | GLN | A | 162 | 55.388 | 60.759 | 3.727 | 1.00 | 81.56 | | C |
| ANISOU | 1242 | CD | GLN | A | 162 | 10670 | 11143 | 9175 | 3858 | 1853 | 1716 | C |
| ATOM | 1243 | OE1 | GLN | A | 162 | 54.371 | 61.371 | 4.102 | 1.00 | 73.74 | | O |
| ANISOU | 1243 | OE1 | GLN | A | 162 | 9713 | 10221 | 8084 | 4145 | 1892 | 1730 | O |
| ATOM | 1244 | NE2 | GLN | A | 162 | 56.419 | 61.394 | 3.184 | 1.00 | 74.93 | | N |
| ANISOU | 1244 | NE2 | GLN | A | 162 | 10154 | 10000 | 8318 | 3791 | 2004 | 1801 | N |
| ATOM | 1245 | C | GLN | A | 162 | 53.573 | 56.303 | 4.049 | 1.00 | 71.77 | | C |
| ANISOU | 1245 | C | GLN | A | 162 | 8076 | 11071 | 8124 | 3437 | 1160 | 1329 | C |
| ATOM | 1246 | O | GLN | A | 162 | 53.949 | 55.871 | 5.138 | 1.00 | 71.81 | | O |
| ANISOU | 1246 | O | GLN | A | 162 | 8028 | 10983 | 8274 | 3127 | 1155 | 1253 | O |
| ATOM | 1247 | N | LYS | A | 163 | 52.292 | 56.238 | 3.641 | 1.00 | 67.35 | | N |
| ANISOU | 1247 | N | LYS | A | 163 | 7314 | 10851 | 7425 | 3727 | 1079 | 1306 | N |
| ATOM | 1248 | CA | LYS | A | 163 | 51.221 | 55.673 | 4.460 | 1.00 | 66.32 | | C |
| ANISOU | 1248 | CA | LYS | A | 163 | 6880 | 11008 | 7310 | 3673 | 994 | 1184 | C |
| ATOM | 1249 | CB | LYS | A | 163 | 49.867 | 56.300 | 4.104 | 1.00 | 68.30 | | C |
| ANISOU | 1249 | CB | LYS | A | 163 | 7019 | 11564 | 7369 | 4106 | 985 | 1196 | C |
| ATOM | 1250 | CG | LYS | A | 163 | 49.544 | 57.477 | 5.009 | 1.00 | 78.26 | | C |
| ANISOU | 1250 | CG | LYS | A | 163 | 8487 | 12650 | 8597 | 4304 | 1148 | 1250 | C |
| ATOM | 1251 | CD | LYS | A | 163 | 49.066 | 58.692 | 4.246 | 1.00 | 91.98 | | C |
| ANISOU | 1251 | CD | LYS | A | 163 | 10445 | 14379 | 10125 | 4811 | 1238 | 1378 | C |
| ATOM | 1252 | CE | LYS | A | 163 | 49.125 | 59.953 | 5.081 | 1.00 | 108.92 | | C |
| ANISOU | 1252 | CE | LYS | A | 163 | 12928 | 16216 | 12242 | 4980 | 1439 | 1456 | C |
| ATOM | 1253 | NZ | LYS | A | 163 | 50.511 | 60.500 | 5.169 | 1.00 | 119.97 | | N |
| ANISOU | 1253 | NZ | LYS | A | 163 | 14721 | 17114 | 13749 | 4753 | 1589 | 1536 | N |
| ATOM | 1254 | C | LYS | A | 163 | 51.185 | 54.156 | 4.414 | 1.00 | 68.21 | | C |
| ANISOU | 1254 | C | LYS | A | 163 | 6819 | 11469 | 7628 | 3361 | 825 | 1057 | C |
| ATOM | 1255 | O | LYS | A | 163 | 50.824 | 53.524 | 5.405 | 1.00 | 67.18 | | O |
| ANISOU | 1255 | O | LYS | A | 163 | 6517 | 11428 | 7582 | 3135 | 790 | 950 | O |
| ATOM | 1256 | N | ARG | A | 164 | 51.595 | 53.572 | 3.288 | 1.00 | 64.02 | | N |
| ANISOU | 1256 | N | ARG | A | 164 | 6254 | 11005 | 7064 | 3340 | 734 | 1069 | N |
| ATOM | 1257 | CA | ARG | A | 164 | 51.638 | 52.122 | 3.117 | 1.00 | 63.58 | | C |
| ANISOU | 1257 | CA | ARG | A | 164 | 5963 | 11129 | 7064 | 3051 | 583 | 951 | C |
| ATOM | 1258 | CB | ARG | A | 164 | 51.829 | 51.741 | 1.630 | 1.00 | 66.79 | | C |
| ANISOU | 1258 | CB | ARG | A | 164 | 6340 | 11665 | 7371 | 3159 | 487 | 977 | C |
| ATOM | 1259 | CG | ARG | A | 164 | 50.680 | 52.267 | 0.743 | 1.00 | 88.83 | | C |
| ANISOU | 1259 | CG | ARG | A | 164 | 9003 | 14791 | 9958 | 3559 | 438 | 991 | C |
| ATOM | 1260 | CD | ARG | A | 164 | 50.480 | 51.544 | −0.588 | 1.00 | 113.61 | | C |
| ANISOU | 1260 | CD | ARG | A | 164 | 12001 | 18186 | 12981 | 3625 | 295 | 958 | C |
| ATOM | 1261 | NE | ARG | A | 164 | 49.125 | 51.770 | −1.117 | 1.00 | 133.82 | | N |
| ANISOU | 1261 | NE | ARG | A | 164 | 14332 | 21169 | 15346 | 3949 | 215 | 914 | N |
| ATOM | 1262 | CZ | ARG | A | 164 | 48.651 | 51.263 | −2.254 | 1.00 | 153.04 | | C |
| ANISOU | 1262 | CZ | ARG | A | 164 | 16592 | 23921 | 17635 | 4065 | 78 | 863 | C |
| ATOM | 1263 | NH1 | ARG | A | 164 | 49.414 | 50.481 | −3.011 | 1.00 | 143.07 | | N |
| ANISOU | 1263 | NH1 | ARG | A | 164 | 15376 | 22582 | 16403 | 3884 | 9 | 855 | N |
| ATOM | 1264 | NH2 | ARG | A | 164 | 47.406 | 51.523 | −2.637 | 1.00 | 137.61 | | N |
| ANISOU | 1264 | NH2 | ARG | A | 164 | 14408 | 22383 | 15494 | 4370 | 5 | 812 | N |
| ATOM | 1265 | C | ARG | A | 164 | 52.696 | 51.489 | 4.035 | 1.00 | 64.06 | | C |
| ANISOU | 1265 | C | ARG | A | 164 | 6103 | 10927 | 7309 | 2654 | 603 | 911 | C |
| ATOM | 1266 | O | ARG | A | 164 | 52.413 | 50.475 | 4.673 | 1.00 | 63.22 | | O |
| ANISOU | 1266 | O | ARG | A | 164 | 5817 | 10941 | 7262 | 2402 | 526 | 794 | O |
| ATOM | 1267 | N | ILE | A | 165 | 53.879 | 52.140 | 4.153 | 1.00 | 58.05 | | N |
| ANISOU | 1267 | N | ILE | A | 165 | 5618 | 9817 | 6623 | 2607 | 717 | 1005 | N |
| ATOM | 1268 | CA | ILE | A | 165 | 55.013 | 51.717 | 4.987 | 1.00 | 55.97 | | C |
| ANISOU | 1268 | CA | ILE | A | 165 | 5450 | 9303 | 6513 | 2276 | 745 | 978 | C |
| ATOM | 1269 | CB | ILE | A | 165 | 56.290 | 52.536 | 4.656 | 1.00 | 58.00 | | C |
| ANISOU | 1269 | CB | ILE | A | 165 | 5988 | 9249 | 6801 | 2282 | 865 | 1086 | C |
| ATOM | 1270 | CG1 | ILE | A | 165 | 56.746 | 52.326 | 3.196 | 1.00 | 58.86 | | C |
| ANISOU | 1270 | CG1 | ILE | A | 165 | 6128 | 9399 | 6837 | 2381 | 821 | 1145 | C |
| ATOM | 1271 | CD1 | ILE | A | 165 | 56.965 | 50.837 | 2.712 | 1.00 | 68.51 | | C |
| ANISOU | 1271 | CD1 | ILE | A | 165 | 7168 | 10784 | 8079 | 2201 | 661 | 1064 | C |
| ATOM | 1272 | CG2 | ILE | A | 165 | 57.422 | 52.269 | 5.628 | 1.00 | 57.01 | | C |
| ANISOU | 1272 | CG2 | ILE | A | 165 | 5950 | 8893 | 6818 | 1971 | 905 | 1051 | C |
| ATOM | 1273 | C | ILE | A | 165 | 54.667 | 51.750 | 6.468 | 1.00 | 59.20 | | C |
| ANISOU | 1273 | C | ILE | A | 165 | 5825 | 9667 | 7001 | 2136 | 788 | 912 | C |
| ATOM | 1274 | O | ILE | A | 165 | 54.943 | 50.780 | 7.168 | 1.00 | 59.51 | | O |
| ANISOU | 1274 | O | ILE | A | 165 | 5787 | 9695 | 7130 | 1858 | 732 | 828 | O |
| ATOM | 1275 | N | ALA | A | 166 | 54.056 | 52.842 | 6.941 | 1.00 | 54.44 | | N |
| ANISOU | 1275 | N | ALA | A | 166 | 5301 | 9033 | 6353 | 2339 | 893 | 953 | N |
| ATOM | 1276 | CA | ALA | A | 166 | 53.675 | 52.979 | 8.344 | 1.00 | 54.24 | | C |
| ANISOU | 1276 | CA | ALA | A | 166 | 5255 | 8965 | 6389 | 2233 | 946 | 894 | C |
| ATOM | 1277 | CB | ALA | A | 166 | 53.128 | 54.373 | 8.608 | 1.00 | 55.19 | | C |
| ANISOU | 1277 | CB | ALA | A | 166 | 5525 | 9010 | 6434 | 2520 | 1081 | 965 | C |
| ATOM | 1278 | C | ALA | A | 166 | 52.668 | 51.903 | 8.797 | 1.00 | 56.79 | | C |
| ANISOU | 1278 | C | ALA | A | 166 | 5284 | 9585 | 6709 | 2111 | 838 | 769 | C |
| ATOM | 1279 | O | ALA | A | 166 | 52.832 | 51.358 | 9.896 | 1.00 | 57.40 | | O |
| ANISOU | 1279 | O | ALA | A | 166 | 5337 | 9594 | 6878 | 1854 | 838 | 697 | O |
| ATOM | 1280 | N | GLN | A | 167 | 51.655 | 51.575 | 7.941 | 1.00 | 50.65 | | N |
| ANISOU | 1280 | N | GLN | A | 167 | 4289 | 9141 | 5816 | 2278 | 750 | 736 | N |
| ATOM | 1281 | CA | GLN | A | 167 | 50.649 | 50.543 | 8.233 | 1.00 | 48.95 | | C |
| ANISOU | 1281 | CA | GLN | A | 167 | 3781 | 9240 | 5579 | 2136 | 655 | 602 | C |
| ATOM | 1282 | CB | GLN | A | 167 | 49.501 | 50.531 | 7.201 | 1.00 | 50.49 | | C |
| ANISOU | 1282 | CB | GLN | A | 167 | 3741 | 9827 | 5617 | 2382 | 572 | 570 | C |
| ATOM | 1283 | CG | GLN | A | 167 | 48.257 | 49.744 | 7.660 | 1.00 | 73.55 | | C |
| ANISOU | 1283 | CG | GLN | A | 167 | 6343 | 13110 | 8494 | 2260 | 514 | 419 | C |
| ATOM | 1284 | CD | GLN | A | 167 | 47.123 | 49.724 | 6.646 | 1.00 | 100.71 | | C |
| ANISOU | 1284 | CD | GLN | A | 167 | 9515 | 16986 | 11764 | 2493 | 423 | 367 | C |
| ATOM | 1285 | OE1 | GLN | A | 167 | 47.324 | 49.536 | 5.436 | 1.00 | 95.58 | | O |
| ANISOU | 1285 | OE1 | GLN | A | 167 | 8857 | 16415 | 11046 | 2583 | 338 | 391 | O |
| ATOM | 1286 | NE2 | GLN | A | 167 | 45.890 | 49.898 | 7.126 | 1.00 | 96.55 | | N |
| ANISOU | 1286 | NE2 | GLN | A | 167 | 8748 | 16781 | 11156 | 2596 | 437 | 286 | N |
| ATOM | 1287 | C | GLN | A | 167 | 51.355 | 49.187 | 8.317 | 1.00 | 48.43 | | C |
| ANISOU | 1287 | C | GLN | A | 167 | 3699 | 9100 | 5603 | 1777 | 573 | 531 | C |
| ATOM | 1288 | O | GLN | A | 167 | 51.282 | 48.555 | 9.373 | 1.00 | 47.73 | | O |
| ANISOU | 1288 | O | GLN | A | 167 | 3576 | 8974 | 5584 | 1529 | 582 | 452 | O |
| ATOM | 1289 | N | GLU | A | 168 | 52.114 | 48.792 | 7.251 | 1.00 | 40.91 | | N |
| ANISOU | 1289 | N | GLU | A | 168 | 2809 | 8095 | 4642 | 1764 | 507 | 569 | N |
| ATOM | 1290 | CA | GLU | A | 168 | 52.896 | 47.554 | 7.237 | 1.00 | 38.87 | | C |
| ANISOU | 1290 | CA | GLU | A | 168 | 2574 | 7745 | 4449 | 1472 | 435 | 515 | C |
| ATOM | 1291 | CB | GLU | A | 168 | 53.714 | 47.408 | 5.952 | 1.00 | 39.58 | | C |
| ANISOU | 1291 | CB | GLU | A | 168 | 2751 | 7777 | 4511 | 1542 | 384 | 580 | C |
| ATOM | 1292 | CG | GLU | A | 168 | 54.806 | 46.360 | 6.057 | 1.00 | 47.65 | | C |
| ANISOU | 1292 | CG | GLU | A | 168 | 3855 | 8646 | 5604 | 1279 | 334 | 546 | C |
| ATOM | 1293 | CD | GLU | A | 168 | 55.074 | 45.541 | 4.813 | 1.00 | 71.87 | | C |
| ANISOU | 1293 | CD | GLU | A | 168 | 6893 | 11808 | 8608 | 1277 | 234 | 533 | C |
| ATOM | 1294 | OE1 | GLU | A | 168 | 56.140 | 45.749 | 4.190 | 1.00 | 61.76 | | O |
| ANISOU | 1294 | OE1 | GLU | A | 168 | 5753 | 10367 | 7344 | 1323 | 248 | 613 | O |
| ATOM | 1295 | OE2 | GLU | A | 168 | 54.232 | 44.677 | 4.474 | 1.00 | 69.13 | | O |
| ANISOU | 1295 | OE2 | GLU | A | 168 | 6381 | 11699 | 8187 | 1211 | 147 | 435 | O |
| ATOM | 1296 | C | GLU | A | 168 | 53.797 | 47.464 | 8.484 | 1.00 | 41.58 | | C |
| ANISOU | 1296 | C | GLU | A | 168 | 3077 | 7804 | 4918 | 1254 | 499 | 515 | C |
| ATOM | 1297 | O | GLU | A | 168 | 53.794 | 46.427 | 9.141 | 1.00 | 40.89 | | O |
| ANISOU | 1297 | O | GLU | A | 168 | 2947 | 7722 | 4866 | 1007 | 461 | 426 | O |
| ATOM | 1298 | N | THR | A | 169 | 54.524 | 48.549 | 8.828 | 1.00 | 37.49 | | N |
| ANISOU | 1298 | N | THR | A | 169 | 2751 | 7044 | 4452 | 1342 | 601 | 607 | N |
| ATOM | 1299 | CA | THR | A | 169 | 55.382 | 48.585 | 10.014 | 1.00 | 37.39 | | C |
| ANISOU | 1299 | CA | THR | A | 169 | 2879 | 6785 | 4542 | 1153 | 660 | 600 | C |
| ATOM | 1300 | CB | THR | A | 169 | 56.048 | 49.956 | 10.167 | 1.00 | 46.28 | | C |
| ANISOU | 1300 | CB | THR | A | 169 | 4207 | 7683 | 5695 | 1275 | 782 | 694 | C |
| ATOM | 1301 | OG1 | THR | A | 169 | 56.833 | 50.230 | 9.015 | 1.00 | 46.07 | | O |
| ANISOU | 1301 | OG1 | THR | A | 169 | 4274 | 7586 | 5645 | 1367 | 784 | 773 | O |
| ATOM | 1302 | CG2 | THR | A | 169 | 56.917 | 50.059 | 11.418 | 1.00 | 44.38 | | C |
| ANISOU | 1302 | CG2 | THR | A | 169 | 4098 | 7215 | 5549 | 1074 | 838 | 672 | C |
| ATOM | 1303 | C | THR | A | 169 | 54.559 | 48.266 | 11.252 | 1.00 | 41.55 | | C |
| ANISOU | 1303 | C | THR | A | 169 | 3314 | 7386 | 5086 | 1033 | 676 | 514 | C |
| ATOM | 1304 | O | THR | A | 169 | 54.944 | 47.397 | 12.033 | 1.00 | 41.21 | | O |
| ANISOU | 1304 | O | THR | A | 169 | 3290 | 7274 | 5093 | 799 | 648 | 452 | O |
| ATOM | 1305 | N | LEU | A | 170 | 53.418 | 48.945 | 11.415 | 1.00 | 38.37 | | N |
| ANISOU | 1305 | N | LEU | A | 170 | 2816 | 7137 | 4628 | 1206 | 725 | 511 | N |
| ATOM | 1306 | CA | LEU | A | 170 | 52.567 | 48.738 | 12.581 | 1.00 | 38.57 | | C |
| ANISOU | 1306 | CA | LEU | A | 170 | 2741 | 7254 | 4658 | 1109 | 758 | 430 | C |
| ATOM | 1307 | CB | LEU | A | 170 | 51.561 | 49.887 | 12.734 | 1.00 | 38.02 | | C |
| ANISOU | 1307 | CB | LEU | A | 170 | 2619 | 7303 | 4522 | 1381 | 840 | 459 | C |
| ATOM | 1308 | CG | LEU | A | 170 | 52.138 | 51.191 | 13.272 | 1.00 | 41.22 | | C |
| ANISOU | 1308 | CG | LEU | A | 170 | 3267 | 7433 | 4961 | 1510 | 964 | 548 | C |
| ATOM | 1309 | CD1 | LEU | A | 170 | 51.184 | 52.322 | 13.067 | 1.00 | 40.80 | | C |
| ANISOU | 1309 | CD1 | LEU | A | 170 | 3196 | 7497 | 4809 | 1845 | 1039 | 594 | C |
| ATOM | 1310 | CD2 | LEU | A | 170 | 52.535 | 51.075 | 14.738 | 1.00 | 42.13 | | C |
| ANISOU | 1310 | CD2 | LEU | A | 170 | 3487 | 7358 | 5164 | 1292 | 1021 | 506 | C |
| ATOM | 1311 | C | LEU | A | 170 | 51.874 | 47.357 | 12.611 | 1.00 | 44.46 | | C |
| ANISOU | 1311 | C | LEU | A | 170 | 3293 | 8226 | 5376 | 902 | 673 | 311 | C |
| ATOM | 1312 | O | LEU | A | 170 | 51.662 | 46.795 | 13.684 | 1.00 | 44.64 | | O |
| ANISOU | 1312 | O | LEU | A | 170 | 3302 | 8231 | 5428 | 702 | 699 | 239 | O |
| ATOM | 1313 | N | GLU | A | 171 | 51.567 | 46.802 | 11.451 | 1.00 | 41.96 | | N |
| ANISOU | 1313 | N | GLU | A | 171 | 2851 | 8100 | 4992 | 932 | 581 | 288 | N |
| ATOM | 1314 | CA | GLU | A | 171 | 50.924 | 45.499 | 11.374 | 1.00 | 42.01 | | C |
| ANISOU | 1314 | CA | GLU | A | 171 | 2694 | 8311 | 4956 | 710 | 511 | 164 | C |
| ATOM | 1315 | CB | GLU | A | 171 | 50.221 | 45.316 | 10.006 | 1.00 | 43.51 | | C |
| ANISOU | 1315 | CB | GLU | A | 171 | 2688 | 8807 | 5036 | 842 | 423 | 133 | C |
| ATOM | 1316 | CG | GLU | A | 171 | 48.974 | 46.175 | 9.801 | 1.00 | 57.10 | | C |
| ANISOU | 1316 | CG | GLU | A | 171 | 4202 | 10829 | 6665 | 1099 | 448 | 123 | C |
| ATOM | 1317 | CD | GLU | A | 171 | 48.041 | 45.853 | 8.640 | 1.00 | 79.12 | | C |
| ANISOU | 1317 | CD | GLU | A | 171 | 6744 | 13999 | 9320 | 1214 | 352 | 59 | C |
| ATOM | 1318 | OE1 | GLU | A | 171 | 48.505 | 45.348 | 7.589 | 1.00 | 58.14 | | O |
| ANISOU | 1318 | OE1 | GLU | A | 171 | 4122 | 11335 | 6635 | 1209 | 266 | 72 | O |
| ATOM | 1319 | OE2 | GLU | A | 171 | 46.827 | 46.126 | 8.789 | 1.00 | 81.42 | | O |
| ANISOU | 1319 | OE2 | GLU | A | 171 | 6796 | 14617 | 9523 | 1321 | 361 | −11 | O |
| ATOM | 1320 | C | GLU | A | 171 | 51.928 | 44.363 | 11.589 | 1.00 | 44.95 | | C |
| ANISOU | 1320 | C | GLU | A | 171 | 3217 | 8479 | 5382 | 444 | 469 | 138 | C |
| ATOM | 1321 | O | GLU | A | 171 | 51.604 | 43.387 | 12.265 | 1.00 | 44.52 | | O |
| ANISOU | 1321 | O | GLU | A | 171 | 3141 | 8452 | 5321 | 199 | 469 | 42 | O |
| ATOM | 1322 | N | ILE | A | 172 | 53.144 | 44.496 | 11.012 | 1.00 | 40.06 | | N |
| ANISOU | 1322 | N | ILE | A | 172 | 2761 | 7660 | 4801 | 502 | 441 | 223 | N |
| ATOM | 1323 | CA | ILE | A | 172 | 54.145 | 43.438 | 10.946 | 1.00 | 38.21 | | C |
| ANISOU | 1323 | CA | ILE | A | 172 | 2661 | 7267 | 4591 | 322 | 387 | 208 | C |
| ATOM | 1324 | CB | ILE | A | 172 | 54.388 | 43.136 | 9.418 | 1.00 | 40.58 | | C |
| ANISOU | 1324 | CB | ILE | A | 172 | 2927 | 7660 | 4831 | 419 | 300 | 231 | C |
| ATOM | 1325 | CG1 | ILE | A | 172 | 53.249 | 42.217 | 8.923 | 1.00 | 40.75 | | C |
| ANISOU | 1325 | CG1 | ILE | A | 172 | 2771 | 7952 | 4759 | 312 | 236 | 115 | C |
| ATOM | 1326 | CD1 | ILE | A | 172 | 53.147 | 41.962 | 7.516 | 1.00 | 50.30 | | C |
| ANISOU | 1326 | CD1 | ILE | A | 172 | 3902 | 9323 | 5888 | 419 | 152 | 115 | C |
| ATOM | 1327 | CG2 | ILE | A | 172 | 55.771 | 42.543 | 9.094 | 1.00 | 40.37 | | C |
| ANISOU | 1327 | CG2 | ILE | A | 172 | 3075 | 7429 | 4834 | 357 | 261 | 269 | C |
| ATOM | 1328 | C | ILE | A | 172 | 55.449 | 43.698 | 11.748 | 1.00 | 39.22 | | C |
| ANISOU | 1328 | C | ILE | A | 172 | 2995 | 7101 | 4804 | 279 | 429 | 267 | C |
| ATOM | 1329 | O | ILE | A | 172 | 55.727 | 42.913 | 12.644 | 1.00 | 39.43 | | O |
| ANISOU | 1329 | O | ILE | A | 172 | 3107 | 7026 | 4848 | 93 | 431 | 216 | O |
| ATOM | 1330 | N | TYR | A | 173 | 56.224 | 44.746 | 11.456 | 1.00 | 33.60 | | N |
| ANISOU | 1330 | N | TYR | A | 173 | 2369 | 6265 | 4132 | 437 | 466 | 364 | N |
| ATOM | 1331 | CA | TYR | A | 173 | 57.543 | 44.948 | 12.057 | 1.00 | 32.75 | | C |
| ANISOU | 1331 | CA | TYR | A | 173 | 2429 | 5923 | 4092 | 381 | 495 | 403 | C |
| ATOM | 1332 | CB | TYR | A | 173 | 58.389 | 45.857 | 11.167 | 1.00 | 33.18 | | C |
| ANISOU | 1332 | CB | TYR | A | 173 | 2552 | 5897 | 4160 | 534 | 521 | 497 | C |
| ATOM | 1333 | CG | TYR | A | 173 | 58.601 | 45.264 | 9.785 | 1.00 | 33.54 | | C |
| ANISOU | 1333 | CG | TYR | A | 173 | 2557 | 6035 | 4152 | 589 | 442 | 514 | C |
| ATOM | 1334 | CD1 | TYR | A | 173 | 59.504 | 44.222 | 9.581 | 1.00 | 34.79 | | C |
| ANISOU | 1334 | CD1 | TYR | A | 173 | 2770 | 6140 | 4307 | 477 | 373 | 490 | C |
| ATOM | 1335 | CE1 | TYR | A | 173 | 59.657 | 43.631 | 8.331 | 1.00 | 34.02 | | C |
| ANISOU | 1335 | CE1 | TYR | A | 173 | 2646 | 6128 | 4152 | 527 | 303 | 498 | C |
| ATOM | 1336 | CZ | TYR | A | 173 | 58.897 | 44.077 | 7.262 | 1.00 | 40.56 | | C |
| ANISOU | 1336 | CZ | TYR | A | 173 | 3382 | 7106 | 4923 | 686 | 292 | 528 | C |
| ATOM | 1337 | OH | TYR | A | 173 | 59.046 | 43.516 | 6.006 | 1.00 | 37.10 | | O |
| ANISOU | 1337 | OH | TYR | A | 173 | 2923 | 6754 | 4418 | 740 | 220 | 533 | O |
| ATOM | 1338 | CE2 | TYR | A | 173 | 57.979 | 45.098 | 7.450 | 1.00 | 34.72 | | C |
| ANISOU | 1338 | CE2 | TYR | A | 173 | 2579 | 6436 | 4178 | 812 | 352 | 552 | C |
| ATOM | 1339 | CD2 | TYR | A | 173 | 57.839 | 45.684 | 8.703 | 1.00 | 33.81 | | C |
| ANISOU | 1339 | CD2 | TYR | A | 173 | 2500 | 6224 | 4123 | 766 | 430 | 546 | C |
| ATOM | 1340 | C | TYR | A | 173 | 57.555 | 45.426 | 13.529 | 1.00 | 38.34 | | C |
| ANISOU | 1340 | C | TYR | A | 173 | 3208 | 6509 | 4852 | 309 | 573 | 386 | C |
| ATOM | 1341 | O | TYR | A | 173 | 58.422 | 44.964 | 14.279 | 1.00 | 39.19 | | O |
| ANISOU | 1341 | O | TYR | A | 173 | 3420 | 6480 | 4988 | 181 | 562 | 365 | O |
| ATOM | 1342 | N | ALA | A | 174 | 56.642 | 46.330 | 13.953 | 1.00 | 34.26 | | N |
| ANISOU | 1342 | N | ALA | A | 174 | 2639 | 6045 | 4333 | 405 | 649 | 393 | N |
| ATOM | 1343 | CA | ALA | A | 174 | 56.601 | 46.747 | 15.357 | 1.00 | 33.83 | | C |
| ANISOU | 1343 | CA | ALA | A | 174 | 2657 | 5879 | 4318 | 335 | 724 | 370 | C |
| ATOM | 1344 | CB | ALA | A | 174 | 55.639 | 47.908 | 15.564 | 1.00 | 34.77 | | C |
| ANISOU | 1344 | CB | ALA | A | 174 | 2734 | 6058 | 4421 | 505 | 815 | 395 | C |
| ATOM | 1345 | C | ALA | A | 174 | 56.199 | 45.523 | 16.209 | 1.00 | 36.94 | | C |
| ANISOU | 1345 | C | ALA | A | 174 | 3024 | 6316 | 4696 | 133 | 692 | 280 | C |
| ATOM | 1346 | O | ALA | A | 174 | 56.941 | 45.234 | 17.152 | 1.00 | 36.99 | | O |
| ANISOU | 1346 | O | ALA | A | 174 | 3153 | 6170 | 4729 | 14 | 699 | 261 | O |
| ATOM | 1347 | N | PRO | A | 175 | 55.169 | 44.694 | 15.835 | 1.00 | 32.30 | | N |
| ANISOU | 1347 | N | PRO | A | 175 | 2295 | 5925 | 4052 | 75 | 655 | 218 | N |
| ATOM | 1348 | CA | PRO | A | 175 | 54.878 | 43.483 | 16.623 | 1.00 | 31.89 | | C |
| ANISOU | 1348 | CA | PRO | A | 175 | 2268 | 5876 | 3974 | −148 | 646 | 132 | C |
| ATOM | 1349 | CB | PRO | A | 175 | 53.581 | 42.960 | 15.997 | 1.00 | 33.21 | | C |
| ANISOU | 1349 | CB | PRO | A | 175 | 2240 | 6304 | 4072 | −186 | 628 | 62 | C |
| ATOM | 1350 | CG | PRO | A | 175 | 52.977 | 44.143 | 15.350 | 1.00 | 37.61 | | C |
| ANISOU | 1350 | CG | PRO | A | 175 | 2656 | 7013 | 4622 | 51 | 646 | 109 | C |
| ATOM | 1351 | CD | PRO | A | 175 | 54.163 | 44.837 | 14.766 | 1.00 | 33.59 | | C |
| ANISOU | 1351 | CD | PRO | A | 175 | 2276 | 6329 | 4158 | 198 | 629 | 212 | C |
| ATOM | 1352 | C | PRO | A | 175 | 55.998 | 42.425 | 16.640 | 1.00 | 35.42 | | C |
| ANISOU | 1352 | C | PRO | A | 175 | 2874 | 6170 | 4415 | −271 | 581 | 125 | C |
| ATOM | 1353 | O | PRO | A | 175 | 56.207 | 41.811 | 17.691 | 1.00 | 34.69 | | O |
| ANISOU | 1353 | O | PRO | A | 175 | 2900 | 5968 | 4312 | −410 | 600 | 87 | O |
| ATOM | 1354 | N | LEU | A | 176 | 56.738 | 42.236 | 15.510 | 1.00 | 30.80 | | N |
| ANISOU | 1354 | N | LEU | A | 176 | 2303 | 5576 | 3823 | −199 | 509 | 166 | N |
| ATOM | 1355 | CA | LEU | A | 176 | 57.871 | 41.308 | 15.454 | 1.00 | 29.46 | | C |
| ANISOU | 1355 | CA | LEU | A | 176 | 2280 | 5276 | 3636 | −265 | 448 | 166 | C |
| ATOM | 1356 | CB | LEU | A | 176 | 58.427 | 41.157 | 14.043 | 1.00 | 28.83 | | C |
| ANISOU | 1356 | CB | LEU | A | 176 | 2176 | 5241 | 3539 | −166 | 379 | 207 | C |
| ATOM | 1357 | CG | LEU | A | 176 | 57.748 | 40.104 | 13.185 | 1.00 | 33.64 | | C |
| ANISOU | 1357 | CG | LEU | A | 176 | 2738 | 5973 | 4071 | −243 | 323 | 148 | C |
| ATOM | 1358 | CD1 | LEU | A | 176 | 58.062 | 40.304 | 11.717 | 1.00 | 33.61 | | C |
| ANISOU | 1358 | CD1 | LEU | A | 176 | 2673 | 6048 | 4048 | −107 | 265 | 194 | C |
| ATOM | 1359 | CD2 | LEU | A | 176 | 58.098 | 38.703 | 13.624 | 1.00 | 35.36 | | C |
| ANISOU | 1359 | CD2 | LEU | A | 176 | 3124 | 6082 | 4230 | −399 | 298 | 93 | C |
| ATOM | 1360 | C | LEU | A | 176 | 58.972 | 41.761 | 16.390 | 1.00 | 34.52 | | C |
| ANISOU | 1360 | C | LEU | A | 176 | 3050 | 5746 | 4322 | −254 | 470 | 197 | C |
| ATOM | 1361 | O | LEU | A | 176 | 59.606 | 40.914 | 17.015 | 1.00 | 34.05 | | O |
| ANISOU | 1361 | O | LEU | A | 176 | 3123 | 5586 | 4229 | −340 | 444 | 169 | O |
| ATOM | 1362 | N | ALA | A | 177 | 59.192 | 43.094 | 16.501 | 1.00 | 32.89 | | N |
| ANISOU | 1362 | N | ALA | A | 177 | 2815 | 5506 | 4177 | −147 | 523 | 249 | N |
| ATOM | 1363 | CA | ALA | A | 177 | 60.184 | 43.686 | 17.402 | 1.00 | 34.02 | | C |
| ANISOU | 1363 | CA | ALA | A | 177 | 3061 | 5507 | 4359 | −156 | 553 | 262 | C |
| ATOM | 1364 | CB | ALA | A | 177 | 60.215 | 45.196 | 17.233 | 1.00 | 34.85 | | C |
| ANISOU | 1364 | CB | ALA | A | 177 | 3143 | 5583 | 4517 | −46 | 628 | 315 | C |
| ATOM | 1365 | C | ALA | A | 177 | 59.826 | 43.336 | 18.837 | 1.00 | 41.16 | | C |
| ANISOU | 1365 | C | ALA | A | 177 | 4035 | 6356 | 5247 | −269 | 587 | 207 | C |
| ATOM | 1366 | O | ALA | A | 177 | 60.685 | 42.860 | 19.582 | 1.00 | 41.05 | | O |
| ANISOU | 1366 | O | ALA | A | 177 | 4135 | 6249 | 5213 | −327 | 559 | 185 | O |
| ATOM | 1367 | N | HIS | A | 178 | 58.537 | 43.502 | 19.195 | 1.00 | 39.29 | | N |
| ANISOU | 1367 | N | HIS | A | 178 | 3724 | 6198 | 5005 | −291 | 645 | 181 | N |
| ATOM | 1368 | CA | HIS | A | 178 | 58.061 | 43.179 | 20.510 | 1.00 | 40.73 | | C |
| ANISOU | 1368 | CA | HIS | A | 178 | 3968 | 6341 | 5167 | −402 | 693 | 129 | C |
| ATOM | 1369 | CB | HIS | A | 178 | 56.572 | 43.493 | 20.654 | 1.00 | 43.34 | | C |
| ANISOU | 1369 | CB | HIS | A | 178 | 4169 | 6808 | 5491 | −401 | 766 | 102 | C |
| ATOM | 1370 | CG | HIS | A | 178 | 56.016 | 43.000 | 21.962 | 1.00 | 48.26 | | C |
| ANISOU | 1370 | CG | HIS | A | 178 | 4856 | 7399 | 6080 | −538 | 827 | 43 | C |
| ATOM | 1371 | ND1 | HIS | A | 178 | 56.605 | 43.348 | 23.178 | 1.00 | 50.45 | | N |
| ANISOU | 1371 | ND1 | HIS | A | 178 | 5270 | 7529 | 6370 | −555 | 861 | 43 | N |
| ATOM | 1372 | CE1 | HIS | A | 178 | 55.910 | 42.702 | 24.104 | 1.00 | 50.82 | | C |
| ANISOU | 1372 | CE1 | HIS | A | 178 | 5362 | 7579 | 6368 | −684 | 915 | −11 | C |
| ATOM | 1373 | NE2 | HIS | A | 178 | 54.926 | 41.968 | 23.567 | 1.00 | 51.32 | | N |
| ANISOU | 1373 | NE2 | HIS | A | 178 | 5324 | 7784 | 6390 | −773 | 923 | −53 | N |
| ATOM | 1374 | CD2 | HIS | A | 178 | 54.994 | 42.140 | 22.199 | 1.00 | 51.25 | | C |
| ANISOU | 1374 | CD2 | HIS | A | 178 | 5195 | 7875 | 6404 | −679 | 859 | −22 | C |
| ATOM | 1375 | C | HIS | A | 178 | 58.339 | 41.729 | 20.881 | 1.00 | 45.06 | | C |
| ANISOU | 1375 | C | HIS | A | 178 | 4638 | 6837 | 5646 | −535 | 648 | 83 | C |
| ATOM | 1376 | O | HIS | A | 178 | 58.858 | 41.464 | 21.967 | 1.00 | 46.78 | | O |
| ANISOU | 1376 | O | HIS | A | 178 | 4994 | 6942 | 5839 | −589 | 657 | 64 | O |
| ATOM | 1377 | N | ARG | A | 179 | 57.977 | 40.800 | 19.989 | 1.00 | 40.10 | | N |
| ANISOU | 1377 | N | ARG | A | 179 | 3978 | 6286 | 4972 | −581 | 604 | 64 | N |
| ATOM | 1378 | CA | ARG | A | 179 | 58.137 | 39.350 | 20.133 | 1.00 | 39.04 | | C |
| ANISOU | 1378 | CA | ARG | A | 179 | 3991 | 6090 | 4752 | −704 | 573 | 20 | C |
| ATOM | 1379 | CB | ARG | A | 179 | 57.726 | 38.665 | 18.805 | 1.00 | 39.01 | | C |
| ANISOU | 1379 | CB | ARG | A | 179 | 3915 | 6196 | 4711 | −729 | 525 | 3 | C |
| ATOM | 1380 | CG | ARG | A | 179 | 57.698 | 37.145 | 18.843 | 1.00 | 45.94 | | C |
| ANISOU | 1380 | CG | ARG | A | 179 | 4970 | 7002 | 5484 | −871 | 511 | −51 | C |
| ATOM | 1381 | CD | ARG | A | 179 | 56.846 | 36.522 | 17.754 | 1.00 | 50.98 | | C |
| ANISOU | 1381 | CD | ARG | A | 179 | 5512 | 7781 | 6077 | −962 | 497 | −102 | C |
| ATOM | 1382 | NE | ARG | A | 179 | 56.965 | 35.063 | 17.812 | 1.00 | 65.48 | | N |
| ANISOU | 1382 | NE | ARG | A | 179 | 7572 | 9506 | 7802 | −1107 | 496 | −155 | N |
| ATOM | 1383 | CZ | ARG | A | 179 | 56.140 | 34.217 | 17.204 | 1.00 | 85.41 | | C |
| ANISOU | 1383 | CZ | ARG | A | 179 | 10086 | 12112 | 10255 | −1271 | 509 | −232 | C |
| ATOM | 1384 | NH1 | ARG | A | 179 | 55.135 | 34.676 | 16.462 | 1.00 | 75.40 | | N |
| ANISOU | 1384 | NH1 | ARG | A | 179 | 8559 | 11077 | 9014 | −1301 | 510 | −272 | N |
| ATOM | 1385 | NH2 | ARG | A | 179 | 56.327 | 32.905 | 17.307 | 1.00 | 71.32 | | N |
| ANISOU | 1385 | NH2 | ARG | A | 179 | 8561 | 10181 | 8356 | −1403 | 525 | −277 | N |
| ATOM | 1386 | C | ARG | A | 179 | 59.578 | 38.987 | 20.501 | 1.00 | 40.20 | | C |
| ANISOU | 1386 | C | ARG | A | 179 | 4311 | 6093 | 4871 | −651 | 514 | 45 | C |
| ATOM | 1387 | O | ARG | A | 179 | 59.809 | 38.152 | 21.373 | 1.00 | 40.58 | | O |
| ANISOU | 1387 | O | ARG | A | 179 | 4539 | 6037 | 4844 | −721 | 521 | 16 | O |
| ATOM | 1388 | N | LEU | A | 180 | 60.522 | 39.667 | 19.871 | 1.00 | 33.96 | | N |
| ANISOU | 1388 | N | LEU | A | 180 | 3463 | 5309 | 4129 | −522 | 465 | 95 | N |
| ATOM | 1389 | CA | LEU | A | 180 | 61.949 | 39.468 | 20.001 | 1.00 | 33.45 | | C |
| ANISOU | 1389 | CA | LEU | A | 180 | 3500 | 5172 | 4038 | −451 | 402 | 112 | C |
| ATOM | 1390 | CB | LEU | A | 180 | 62.576 | 39.941 | 18.684 | 1.00 | 32.88 | | C |
| ANISOU | 1390 | CB | LEU | A | 180 | 3320 | 5166 | 4008 | −343 | 356 | 159 | C |
| ATOM | 1391 | CG | LEU | A | 180 | 62.867 | 38.924 | 17.576 | 1.00 | 35.60 | | C |
| ANISOU | 1391 | CG | LEU | A | 180 | 3699 | 5539 | 4289 | −312 | 288 | 163 | C |
| ATOM | 1392 | CD1 | LEU | A | 180 | 61.654 | 38.076 | 17.200 | 1.00 | 34.30 | | C |
| ANISOU | 1392 | CD1 | LEU | A | 180 | 3537 | 5416 | 4082 | −412 | 303 | 126 | C |
| ATOM | 1393 | CD2 | LEU | A | 180 | 63.350 | 39.643 | 16.362 | 1.00 | 38.22 | | C |
| ANISOU | 1393 | CD2 | LEU | A | 180 | 3910 | 5940 | 4670 | −205 | 265 | 214 | C |
| ATOM | 1394 | C | LEU | A | 180 | 62.580 | 40.209 | 21.182 | 1.00 | 40.02 | | C |
| ANISOU | 1394 | C | LEU | A | 180 | 4372 | 5942 | 4893 | −437 | 424 | 106 | C |
| ATOM | 1395 | O | LEU | A | 180 | 63.809 | 40.220 | 21.316 | 1.00 | 40.53 | | O |
| ANISOU | 1395 | O | LEU | A | 180 | 4477 | 5989 | 4935 | −373 | 370 | 109 | O |
| ATOM | 1396 | N | GLY | A | 181 | 61.751 | 40.830 | 22.009 | 1.00 | 38.21 | | N |
| ANISOU | 1396 | N | GLY | A | 181 | 4120 | 5699 | 4698 | −494 | 502 | 90 | N |
| ATOM | 1397 | CA | GLY | A | 181 | 62.201 | 41.577 | 23.177 | 1.00 | 39.46 | | C |
| ANISOU | 1397 | CA | GLY | A | 181 | 4323 | 5796 | 4873 | −496 | 533 | 75 | C |
| ATOM | 1398 | C | GLY | A | 181 | 62.965 | 42.854 | 22.879 | 1.00 | 47.74 | | C |
| ANISOU | 1398 | C | GLY | A | 181 | 5290 | 6854 | 5994 | −431 | 536 | 99 | C |
| ATOM | 1399 | O | GLY | A | 181 | 63.703 | 43.327 | 23.751 | 1.00 | 48.78 | | O |
| ANISOU | 1399 | O | GLY | A | 181 | 5472 | 6945 | 6118 | −441 | 533 | 72 | O |
| ATOM | 1400 | N | MET | A | 182 | 62.753 | 43.445 | 21.663 | 1.00 | 45.02 | | N |
| ANISOU | 1400 | N | MET | A | 182 | 4830 | 6566 | 5710 | −373 | 552 | 145 | N |
| ATOM | 1401 | CA | MET | A | 182 | 63.386 | 44.666 | 21.126 | 1.00 | 45.50 | | C |
| ANISOU | 1401 | CA | MET | A | 182 | 4835 | 6621 | 5832 | −321 | 580 | 177 | C |
| ATOM | 1402 | CB | MET | A | 182 | 63.543 | 44.547 | 19.587 | 1.00 | 47.53 | | C |
| ANISOU | 1402 | CB | MET | A | 182 | 5013 | 6946 | 6100 | −247 | 544 | 227 | C |
| ATOM | 1403 | CG | MET | A | 182 | 64.219 | 43.304 | 19.130 | 1.00 | 50.99 | | C |
| ANISOU | 1403 | CG | MET | A | 182 | 5477 | 7421 | 6476 | −240 | 447 | 216 | C |
| ATOM | 1404 | SD | MET | A | 182 | 65.949 | 43.361 | 19.581 | 1.00 | 55.10 | | S |
| ANISOU | 1404 | SD | MET | A | 182 | 6028 | 7939 | 6968 | −239 | 398 | 186 | S |
| ATOM | 1405 | CE | MET | A | 182 | 66.582 | 44.065 | 18.154 | 1.00 | 51.85 | | C |
| ANISOU | 1405 | CE | MET | A | 182 | 5520 | 7580 | 6600 | −180 | 411 | 237 | C |
| ATOM | 1406 | C | MET | A | 182 | 62.573 | 45.945 | 21.410 | 1.00 | 52.31 | | C |
| ANISOU | 1406 | C | MET | A | 182 | 5679 | 7447 | 6749 | −302 | 688 | 193 | C |
| ATOM | 1407 | O | MET | A | 182 | 62.146 | 46.611 | 20.457 | 1.00 | 54.15 | | O |
| ANISOU | 1407 | O | MET | A | 182 | 5851 | 7709 | 7016 | −220 | 728 | 244 | O |
| ATOM | 1408 | N | GLY | A | 183 | 62.403 | 46.296 | 22.682 | 1.00 | 48.36 | | N |
| ANISOU | 1408 | N | GLY | A | 183 | 5247 | 6882 | 6244 | −355 | 736 | 154 | N |
| ATOM | 1409 | CA | GLY | A | 183 | 61.628 | 47.460 | 23.111 | 1.00 | 48.66 | | C |
| ANISOU | 1409 | CA | GLY | A | 183 | 5298 | 6873 | 6316 | −325 | 845 | 162 | C |
| ATOM | 1410 | C | GLY | A | 183 | 61.993 | 48.828 | 22.551 | 1.00 | 54.30 | | C |
| ANISOU | 1410 | C | GLY | A | 183 | 6031 | 7526 | 7074 | −267 | 918 | 201 | C |
| ATOM | 1411 | O | GLY | A | 183 | 61.103 | 49.631 | 22.250 | 1.00 | 53.93 | | O |
| ANISOU | 1411 | O | GLY | A | 183 | 5974 | 7475 | 7041 | −168 | 1000 | 240 | O |
| ATOM | 1412 | N | GLN | A | 184 | 63.297 | 49.119 | 22.425 | 1.00 | 52.46 | | N |
| ANISOU | 1412 | N | GLN | A | 184 | 5835 | 7250 | 6848 | −324 | 899 | 185 | N |
| ATOM | 1413 | CA | GLN | A | 184 | 63.786 | 50.408 | 21.912 | 1.00 | 53.25 | | C |
| ANISOU | 1413 | CA | GLN | A | 184 | 5986 | 7268 | 6978 | −311 | 988 | 213 | C |
| ATOM | 1414 | CB | GLN | A | 184 | 65.253 | 50.628 | 22.311 | 1.00 | 55.49 | | C |
| ANISOU | 1414 | CB | GLN | A | 184 | 6303 | 7527 | 7256 | −442 | 969 | 152 | C |
| ATOM | 1415 | CG | GLN | A | 184 | 65.434 | 50.987 | 23.797 | 1.00 | 81.94 | | C |
| ANISOU | 1415 | CG | GLN | A | 184 | 9737 | 10810 | 10584 | −543 | 994 | 71 | C |
| ATOM | 1416 | CD | GLN | A | 184 | 65.298 | 52.466 | 24.062 | 1.00 | 103.93 | | C |
| ANISOU | 1416 | CD | GLN | A | 184 | 12651 | 13451 | 13388 | −572 | 1138 | 67 | C |
| ATOM | 1417 | OE1 | GLN | A | 184 | 64.221 | 52.967 | 24.422 | 1.00 | 102.96 | | O |
| ANISOU | 1417 | OE1 | GLN | A | 184 | 12599 | 13255 | 13269 | −494 | 1220 | 92 | O |
| ATOM | 1418 | NE2 | GLN | A | 184 | 66.399 | 53.192 | 23.893 | 1.00 | 90.49 | | N |
| ANISOU | 1418 | NE2 | GLN | A | 184 | 10989 | 11708 | 11687 | −688 | 1180 | 28 | N |
| ATOM | 1419 | C | GLN | A | 184 | 63.599 | 50.540 | 20.400 | 1.00 | 57.08 | | C |
| ANISOU | 1419 | C | GLN | A | 184 | 6415 | 7796 | 7476 | −200 | 996 | 292 | C |
| ATOM | 1420 | O | GLN | A | 184 | 63.629 | 51.655 | 19.856 | 1.00 | 58.30 | | O |
| ANISOU | 1420 | O | GLN | A | 184 | 6639 | 7870 | 7642 | −147 | 1096 | 336 | O |
| ATOM | 1421 | N | LEU | A | 185 | 63.429 | 49.390 | 19.719 | 1.00 | 50.27 | | N |
| ANISOU | 1421 | N | LEU | A | 185 | 5451 | 7051 | 6597 | −164 | 895 | 307 | N |
| ATOM | 1422 | CA | LEU | A | 185 | 63.160 | 49.338 | 18.297 | 1.00 | 47.64 | | C |
| ANISOU | 1422 | CA | LEU | A | 185 | 5055 | 6784 | 6263 | −52 | 883 | 375 | C |
| ATOM | 1423 | CB | LEU | A | 185 | 63.624 | 48.007 | 17.696 | 1.00 | 46.32 | | C |
| ANISOU | 1423 | CB | LEU | A | 185 | 4810 | 6718 | 6070 | −71 | 761 | 366 | C |
| ATOM | 1424 | CG | LEU | A | 185 | 64.554 | 48.103 | 16.489 | 1.00 | 48.25 | | C |
| ANISOU | 1424 | CG | LEU | A | 185 | 5033 | 6987 | 6314 | −38 | 746 | 406 | C |
| ATOM | 1425 | CD1 | LEU | A | 185 | 65.798 | 48.928 | 16.784 | 1.00 | 47.22 | | C |
| ANISOU | 1425 | CD1 | LEU | A | 185 | 4957 | 6783 | 6201 | −129 | 801 | 383 | C |
| ATOM | 1426 | CD2 | LEU | A | 185 | 64.992 | 46.752 | 16.078 | 1.00 | 49.06 | | C |
| ANISOU | 1426 | CD2 | LEU | A | 185 | 5080 | 7181 | 6378 | −44 | 629 | 389 | C |
| ATOM | 1427 | C | LEU | A | 185 | 61.672 | 49.538 | 18.155 | 1.00 | 52.08 | | C |
| ANISOU | 1427 | C | LEU | A | 185 | 5576 | 7399 | 6814 | 65 | 921 | 404 | C |
| ATOM | 1428 | O | LEU | A | 185 | 61.270 | 50.513 | 17.525 | 1.00 | 52.57 | | O |
| ANISOU | 1428 | O | LEU | A | 185 | 5664 | 7439 | 6871 | 193 | 997 | 463 | O |
| ATOM | 1429 | N | LYS | A | 186 | 60.859 | 48.697 | 18.843 | 1.00 | 48.42 | | N |
| ANISOU | 1429 | N | LYS | A | 186 | 5061 | 7002 | 6333 | 24 | 882 | 359 | N |
| ATOM | 1430 | CA | LYS | A | 186 | 59.389 | 48.766 | 18.866 | 1.00 | 48.46 | | C |
| ANISOU | 1430 | CA | LYS | A | 186 | 4990 | 7107 | 6318 | 111 | 916 | 362 | C |
| ATOM | 1431 | C | LYS | A | 186 | 58.873 | 50.174 | 19.165 | 1.00 | 54.00 | | C |
| ANISOU | 1431 | C | LYS | A | 186 | 5759 | 7736 | 7023 | 227 | 1039 | 393 | C |
| ATOM | 1432 | O | LYS | A | 186 | 57.985 | 50.646 | 18.455 | 1.00 | 54.01 | | O |
| ANISOU | 1432 | O | LYS | A | 186 | 5702 | 7823 | 6995 | 394 | 1074 | 437 | O |
| ATOM | 1433 | CB | LYS | A | 186 | 58.789 | 47.756 | 19.871 | 1.00 | 50.08 | | C |
| ANISOU | 1433 | CB | LYS | A | 186 | 5164 | 7363 | 6501 | −7 | 884 | 293 | C |
| ATOM | 1434 | CG | LYS | A | 186 | 57.259 | 47.681 | 19.872 | 1.00 | 50.82 | | C |
| ANISOU | 1434 | CG | LYS | A | 186 | 5139 | 7606 | 6565 | 51 | 920 | 277 | C |
| ATOM | 1435 | CD | LYS | A | 186 | 56.731 | 47.414 | 21.259 | 1.00 | 59.17 | | C |
| ANISOU | 1435 | CD | LYS | A | 186 | 6225 | 8648 | 7609 | −60 | 960 | 215 | C |
| ATOM | 1436 | CE | LYS | A | 186 | 55.232 | 47.563 | 21.319 | 1.00 | 80.17 | | C |
| ANISOU | 1436 | CE | LYS | A | 186 | 8749 | 11476 | 10235 | 3 | 1018 | 193 | C |
| ATOM | 1437 | NZ | LYS | A | 186 | 54.805 | 48.539 | 22.371 | 1.00 | 96.19 | | N |
| ANISOU | 1437 | NZ | LYS | A | 186 | 10837 | 13442 | 12268 | 64 | 1126 | 189 | N |
| ATOM | 1438 | N | TRP | A | 187 | 59.429 | 50.838 | 20.198 | 1.00 | 51.87 | | N |
| ANISOU | 1438 | N | TRP | A | 187 | 5621 | 7313 | 6774 | 153 | 1103 | 368 | N |
| ATOM | 1439 | CA | TRP | A | 187 | 59.004 | 52.179 | 20.584 | 1.00 | 52.72 | | C |
| ANISOU | 1439 | CA | TRP | A | 187 | 5839 | 7317 | 6873 | 254 | 1233 | 390 | C |
| ATOM | 1440 | CB | TRP | A | 187 | 59.801 | 52.707 | 21.815 | 1.00 | 53.84 | | C |
| ANISOU | 1440 | CB | TRP | A | 187 | 6132 | 7290 | 7033 | 115 | 1286 | 337 | C |
| ATOM | 1441 | CG | TRP | A | 187 | 59.859 | 54.215 | 21.902 | 1.00 | 56.97 | | C |
| ANISOU | 1441 | CG | TRP | A | 187 | 6707 | 7520 | 7419 | 191 | 1429 | 366 | C |
| ATOM | 1442 | CD1 | TRP | A | 187 | 58.820 | 55.066 | 22.167 | 1.00 | 60.37 | | C |
| ANISOU | 1442 | CD1 | TRP | A | 187 | 7205 | 7920 | 7814 | 357 | 1537 | 393 | C |
| ATOM | 1443 | NE1 | TRP | A | 187 | 59.237 | 56.377 | 22.057 | 1.00 | 60.54 | | N |
| ANISOU | 1443 | NE1 | TRP | A | 187 | 7439 | 7746 | 7817 | 401 | 1666 | 423 | N |
| ATOM | 1444 | CE2 | TRP | A | 187 | 60.560 | 56.395 | 21.684 | 1.00 | 62.25 | | C |
| ANISOU | 1444 | CE2 | TRP | A | 187 | 7707 | 7881 | 8063 | 238 | 1646 | 411 | C |
| ATOM | 1445 | CD2 | TRP | A | 187 | 60.990 | 55.050 | 21.588 | 1.00 | 57.51 | | C |
| ANISOU | 1445 | CD2 | TRP | A | 187 | 6921 | 7434 | 7498 | 118 | 1492 | 375 | C |
| ATOM | 1446 | CE3 | TRP | A | 187 | 62.327 | 54.791 | 21.229 | 1.00 | 59.20 | | C |
| ANISOU | 1446 | CE3 | TRP | A | 187 | 7127 | 7632 | 7736 | −38 | 1442 | 351 | C |
| ATOM | 1447 | CZ3 | TRP | A | 187 | 63.172 | 55.862 | 20.962 | 1.00 | 61.01 | | C |
| ANISOU | 1447 | CZ3 | TRP | A | 187 | 7515 | 7705 | 7960 | −104 | 1550 | 356 | C |
| ATOM | 1448 | CH2 | TRP | A | 187 | 62.727 | 57.185 | 21.088 | 1.00 | 61.85 | | C |
| ANISOU | 1448 | CH2 | TRP | A | 187 | 7831 | 7635 | 8034 | −15 | 1713 | 389 | C |
| ATOM | 1449 | CZ2 | TRP | A | 187 | 61.419 | 57.477 | 21.426 | 1.00 | 61.90 | | C |
| ANISOU | 1449 | CZ2 | TRP | A | 187 | 7866 | 7644 | 8010 | 177 | 1761 | 422 | C |
| ATOM | 1450 | C | TRP | A | 187 | 59.098 | 53.129 | 19.397 | 1.00 | 50.69 | | C |
| ANISOU | 1450 | C | TRP | A | 187 | 5645 | 7015 | 6602 | 414 | 1297 | 473 | C |
| ATOM | 1451 | O | TRP | A | 187 | 58.102 | 53.739 | 19.012 | 1.00 | 49.70 | | O |
| ANISOU | 1451 | O | TRP | A | 187 | 5511 | 6939 | 6433 | 613 | 1359 | 518 | O |
| ATOM | 1452 | N | GLU | A | 188 | 60.288 | 53.195 | 18.796 | 1.00 | 44.03 | | N |
| ANISOU | 1452 | N | GLU | A | 188 | 4856 | 6096 | 5779 | 336 | 1283 | 490 | N |
| ATOM | 1453 | CA | GLU | A | 188 | 60.615 | 54.086 | 17.702 | 1.00 | 42.32 | | C |
| ANISOU | 1453 | CA | GLU | A | 188 | 4737 | 5801 | 5543 | 446 | 1358 | 566 | C |
| ATOM | 1454 | CB | GLU | A | 188 | 62.113 | 54.101 | 17.461 | 1.00 | 43.20 | | C |
| ANISOU | 1454 | CB | GLU | A | 188 | 4905 | 5825 | 5683 | 275 | 1354 | 550 | C |
| ATOM | 1455 | CG | GLU | A | 188 | 62.589 | 55.471 | 17.033 | 1.00 | 54.05 | | C |
| ANISOU | 1455 | CG | GLU | A | 188 | 6488 | 7011 | 7038 | 298 | 1510 | 595 | C |
| ATOM | 1456 | CD | GLU | A | 188 | 64.093 | 55.608 | 16.942 | 1.00 | 81.98 | | C |
| ANISOU | 1456 | CD | GLU | A | 188 | 10068 | 10482 | 10598 | 91 | 1525 | 558 | C |
| ATOM | 1457 | OE1 | GLU | A | 188 | 64.806 | 55.031 | 17.799 | 1.00 | 81.17 | | O |
| ANISOU | 1457 | OE1 | GLU | A | 188 | 9892 | 10424 | 10526 | −92 | 1450 | 470 | O |
| ATOM | 1458 | OE2 | GLU | A | 188 | 64.554 | 56.308 | 16.011 | 1.00 | 73.56 | | O |
| ANISOU | 1458 | OE2 | GLU | A | 188 | 9109 | 9332 | 9508 | 119 | 1618 | 616 | O |
| ATOM | 1459 | C | GLU | A | 188 | 59.871 | 53.755 | 16.454 | 1.00 | 46.62 | | C |
| ANISOU | 1459 | C | GLU | A | 188 | 5165 | 6501 | 6048 | 631 | 1308 | 629 | C |
| ATOM | 1460 | O | GLU | A | 188 | 59.453 | 54.667 | 15.749 | 1.00 | 46.04 | | O |
| ANISOU | 1460 | O | GLU | A | 188 | 5179 | 6390 | 5925 | 824 | 1394 | 702 | O |
| ATOM | 1461 | N | LEU | A | 189 | 59.697 | 52.454 | 16.172 | 1.00 | 43.98 | | N |
| ANISOU | 1461 | N | LEU | A | 189 | 4650 | 6339 | 5721 | 579 | 1173 | 599 | N |
| ATOM | 1462 | CA | LEU | A | 189 | 58.957 | 51.995 | 15.004 | 1.00 | 43.53 | | C |
| ANISOU | 1462 | CA | LEU | A | 189 | 4460 | 6461 | 5618 | 730 | 1108 | 637 | C |
| ATOM | 1463 | CB | LEU | A | 189 | 59.006 | 50.463 | 14.856 | 1.00 | 43.05 | | C |
| ANISOU | 1463 | CB | LEU | A | 189 | 4244 | 6546 | 5566 | 605 | 966 | 582 | C |
| ATOM | 1464 | CG | LEU | A | 189 | 60.303 | 49.868 | 14.321 | 1.00 | 46.62 | | C |
| ANISOU | 1464 | CG | LEU | A | 189 | 4715 | 6957 | 6040 | 495 | 900 | 584 | C |
| ATOM | 1465 | CD1 | LEU | A | 189 | 60.385 | 48.415 | 14.658 | 1.00 | 46.84 | | C |
| ANISOU | 1465 | CD1 | LEU | A | 189 | 4656 | 7071 | 6068 | 357 | 785 | 516 | C |
| ATOM | 1466 | CD2 | LEU | A | 189 | 60.411 | 50.028 | 12.822 | 1.00 | 47.31 | | C |
| ANISOU | 1466 | CD2 | LEU | A | 189 | 4785 | 7099 | 6090 | 634 | 889 | 654 | C |
| ATOM | 1467 | C | LEU | A | 189 | 57.524 | 52.499 | 15.105 | 1.00 | 48.31 | | C |
| ANISOU | 1467 | C | LEU | A | 189 | 5013 | 7173 | 6168 | 927 | 1157 | 650 | C |
| ATOM | 1468 | O | LEU | A | 189 | 57.086 | 53.188 | 14.189 | 1.00 | 48.93 | | O |
| ANISOU | 1468 | O | LEU | A | 189 | 5114 | 7290 | 6186 | 1148 | 1198 | 720 | O |
| ATOM | 1469 | N | GLU | A | 190 | 56.839 | 52.251 | 16.248 | 1.00 | 43.86 | | N |
| ANISOU | 1469 | N | GLU | A | 190 | 4398 | 6652 | 5615 | 866 | 1166 | 587 | N |
| ATOM | 1470 | CA | GLU | A | 190 | 55.469 | 52.700 | 16.489 | 1.00 | 43.99 | | C |
| ANISOU | 1470 | CA | GLU | A | 190 | 4341 | 6801 | 5574 | 1047 | 1218 | 585 | C |
| ATOM | 1471 | CB | GLU | A | 190 | 54.997 | 52.311 | 17.900 | 1.00 | 46.11 | | C |
| ANISOU | 1471 | CB | GLU | A | 190 | 4569 | 7085 | 5865 | 910 | 1232 | 504 | C |
| ATOM | 1472 | CG | GLU | A | 190 | 54.619 | 50.849 | 18.105 | 1.00 | 62.11 | | C |
| ANISOU | 1472 | CG | GLU | A | 190 | 6416 | 9283 | 7901 | 731 | 1127 | 426 | C |
| ATOM | 1473 | CD | GLU | A | 190 | 53.926 | 50.519 | 19.421 | 1.00 | 89.31 | | C |
| ANISOU | 1473 | CD | GLU | A | 190 | 9816 | 12772 | 11345 | 620 | 1161 | 351 | C |
| ATOM | 1474 | OE1 | GLU | A | 190 | 54.124 | 51.252 | 20.418 | 1.00 | 92.16 | | O |
| ANISOU | 1474 | OE1 | GLU | A | 190 | 10313 | 12980 | 11722 | 619 | 1249 | 349 | O |
| ATOM | 1475 | OE2 | GLU | A | 190 | 53.182 | 49.513 | 19.452 | 1.00 | 85.54 | | O |
| ANISOU | 1475 | OE2 | GLU | A | 190 | 9177 | 12482 | 10843 | 521 | 1108 | 288 | O |
| ATOM | 1476 | C | GLU | A | 190 | 55.314 | 54.217 | 16.294 | 1.00 | 47.47 | | C |
| ANISOU | 1476 | C | GLU | A | 190 | 4961 | 7110 | 5964 | 1283 | 1353 | 663 | C |
| ATOM | 1477 | O | GLU | A | 190 | 54.523 | 54.633 | 15.449 | 1.00 | 47.59 | | O |
| ANISOU | 1477 | O | GLU | A | 190 | 4925 | 7260 | 5897 | 1539 | 1367 | 712 | O |
| ATOM | 1478 | N | ASP | A | 191 | 56.093 | 55.028 | 17.046 | 1.00 | 43.27 | | N |
| ANISOU | 1478 | N | ASP | A | 191 | 4655 | 6319 | 5469 | 1198 | 1454 | 669 | N |
| ATOM | 1479 | CA | ASP | A | 191 | 56.064 | 56.500 | 17.028 | 1.00 | 43.25 | | C |
| ANISOU | 1479 | CA | ASP | A | 191 | 4893 | 6128 | 5413 | 1381 | 1610 | 733 | C |
| ATOM | 1480 | C | ASP | A | 191 | 56.349 | 57.146 | 15.641 | 1.00 | 48.01 | | C |
| ANISOU | 1480 | C | ASP | A | 191 | 5611 | 6676 | 5956 | 1565 | 1653 | 833 | C |
| ATOM | 1481 | O | ASP | A | 191 | 55.756 | 58.178 | 15.319 | 1.00 | 46.84 | | O |
| ANISOU | 1481 | O | ASP | A | 191 | 5601 | 6482 | 5714 | 1838 | 1759 | 901 | O |
| ATOM | 1482 | CB | ASP | A | 191 | 57.013 | 57.080 | 18.105 | 1.00 | 45.28 | | C |
| ANISOU | 1482 | CB | ASP | A | 191 | 5366 | 6117 | 5720 | 1181 | 1700 | 695 | C |
| ATOM | 1483 | CG | ASP | A | 191 | 56.458 | 57.179 | 19.535 | 1.00 | 59.73 | | C |
| ANISOU | 1483 | CG | ASP | A | 191 | 7203 | 7936 | 7558 | 1136 | 1739 | 627 | C |
| ATOM | 1484 | OD1 | ASP | A | 191 | 55.487 | 56.427 | 19.866 | 1.00 | 60.23 | | O |
| ANISOU | 1484 | OD1 | ASP | A | 191 | 7055 | 8220 | 7612 | 1174 | 1671 | 590 | O |
| ATOM | 1485 | OD2 | ASP | A | 191 | 57.001 | 58.000 | 20.332 | 1.00 | 65.04 | | O |
| ANISOU | 1485 | OD2 | ASP | A | 191 | 8093 | 8382 | 8238 | 1047 | 1844 | 605 | O |
| ATOM | I486 | N | LEU | A | 192 | 57.259 | 56.563 | 14.836 | 1.00 | 45.79 | | N |
| ANISOU | I486 | N | LEU | A | 192 | 5292 | 6393 | 5714 | 1434 | 1580 | 846 | N |
| ATOM | 1487 | CA | LEU | A | 192 | 57.546 | 57.111 | 13.511 | 1.00 | 46.19 | | C |
| ANISOU | 1487 | CA | LEU | A | 192 | 5454 | 6394 | 5702 | 1596 | 1623 | 940 | C |
| ATOM | 1488 | CB | LEU | A | 192 | 58.922 | 56.701 | 12.953 | 1.00 | 46.40 | | C |
| ANISOU | 1488 | CB | LEU | A | 192 | 5498 | 6342 | 5788 | 1379 | 1585 | 940 | C |
| ATOM | 1489 | CG | LEU | A | 192 | 60.122 | 57.500 | 13.460 | 1.00 | 51.33 | | C |
| ANISOU | 1489 | CG | LEU | A | 192 | 6359 | 6692 | 6452 | 1181 | 1712 | 929 | C |
| ATOM | 1490 | CD1 | LEU | A | 192 | 61.376 | 56.647 | 13.494 | 1.00 | 51.69 | | C |
| ANISOU | 1490 | CD1 | LEU | A | 192 | 6301 | 6759 | 6580 | 897 | 1623 | 868 | C |
| ATOM | 1491 | CD2 | LEU | A | 192 | 60.362 | 58.719 | 12.613 | 1.00 | 53.76 | | C |
| ANISOU | 1491 | CD2 | LEU | A | 192 | 6935 | 6811 | 6680 | 1326 | 1873 | 1026 | C |
| ATOM | 1492 | C | LEU | A | 192 | 56.453 | 56.699 | 12.568 | 1.00 | 51.36 | | C |
| ANISOU | 1492 | C | LEU | A | 192 | 5920 | 7319 | 6275 | 1850 | 1537 | 973 | C |
| ATOM | 1493 | O | LEU | A | 192 | 56.178 | 57.437 | 11.641 | 1.00 | 51.98 | | O |
| ANISOU | 1493 | O | LEU | A | 192 | 6114 | 7381 | 6256 | 2106 | 1600 | 1062 | O |
| ATOM | 1494 | N | SER | A | 193 | 55.805 | 55.544 | 12.815 | 1.00 | 48.09 | | N |
| ANISOU | 1494 | N | SER | A | 193 | 5229 | 7158 | 5885 | 1778 | 1400 | 897 | N |
| ATOM | 1495 | CA | SER | A | 193 | 54.700 | 55.052 | 11.996 | 1.00 | 48.07 | | C |
| ANISOU | 1495 | CA | SER | A | 193 | 5003 | 7461 | 5799 | 1977 | 1306 | 897 | C |
| ATOM | 1496 | CB | SER | A | 193 | 54.409 | 53.597 | 12.316 | 1.00 | 50.16 | | C |
| ANISOU | 1496 | CB | SER | A | 193 | 5004 | 7938 | 6115 | 1761 | 1163 | 794 | C |
| ATOM | 1497 | OG | SER | A | 193 | 55.393 | 52.769 | 11.727 | 1.00 | 56.46 | | O |
| ANISOU | 1497 | OG | SER | A | 193 | 5794 | 8692 | 6968 | 1575 | 1079 | 788 | O |
| ATOM | 1498 | C | SER | A | 193 | 53.475 | 55.905 | 12.241 | 1.00 | 54.29 | | C |
| ANISOU | 1498 | C | SER | A | 193 | 5792 | 8349 | 6486 | 2273 | 1383 | 917 | C |
| ATOM | 1499 | O | SER | A | 193 | 52.792 | 56.297 | 11.305 | 1.00 | 52.96 | | O |
| ANISOU | 1499 | O | SER | A | 193 | 5586 | 8335 | 6200 | 2572 | 1378 | 972 | O |
| ATOM | 1500 | N | PHE | A | 194 | 53.255 | 56.265 | 13.501 | 1.00 | 54.45 | | N |
| ANISOU | 1500 | N | PHE | A | 194 | 5875 | 8273 | 6541 | 2212 | 1460 | 877 | N |
| ATOM | 1501 | CA | PHE | A | 194 | 52.171 | 57.135 | 13.948 | 1.00 | 55.49 | | C |
| ANISOU | 1501 | CA | PHE | A | 194 | 6032 | 8473 | 6578 | 2488 | 1552 | 891 | C |
| ATOM | 1502 | C | PHE | A | 194 | 52.288 | 58.514 | 13.291 | 1.00 | 61.40 | | C |
| ANISOU | 1502 | C | PHE | A | 194 | 7079 | 9031 | 7220 | 2797 | 1688 | 1010 | C |
| ATOM | 1503 | O | PHE | A | 194 | 51.269 | 59.105 | 12.959 | 1.00 | 61.71 | | O |
| ANISOU | 1503 | O | PHE | A | 194 | 7095 | 9228 | 7124 | 3152 | 1722 | 1049 | O |
| ATOM | 1504 | CB | PHE | A | 194 | 52.220 | 57.239 | 15.482 | 1.00 | 57.30 | | C |
| ANISOU | 1504 | CB | PHE | A | 194 | 6322 | 8572 | 6879 | 2306 | 1618 | 825 | C |
| ATOM | 1505 | CG | PHE | A | 194 | 51.246 | 58.151 | 16.182 | 1.00 | 59.33 | | C |
| ANISOU | 1505 | CG | PHE | A | 194 | 6643 | 8847 | 7051 | 2552 | 1734 | 830 | C |
| ATOM | 1506 | CD1 | PHE | A | 194 | 49.969 | 58.361 | 15.672 | 1.00 | 63.08 | | C |
| ANISOU | 1506 | CD1 | PHE | A | 194 | 6953 | 9619 | 7395 | 2893 | 1723 | 844 | C |
| ATOM | 1507 | CD2 | PHE | A | 194 | 51.567 | 58.718 | 17.411 | 1.00 | 61.77 | | C |
| ANISOU | 1507 | CD2 | PHE | A | 194 | 7153 | 8913 | 7403 | 2442 | 1844 | 805 | C |
| ATOM | 1508 | CE1 | PHE | A | 194 | 49.062 | 59.192 | 16.334 | 1.00 | 64.36 | | C |
| ANISOU | 1508 | CE1 | PHE | A | 194 | 7169 | 9819 | 7466 | 3148 | 1833 | 847 | C |
| ATOM | 1509 | CE2 | PHE | A | 194 | 50.661 | 59.540 | 18.076 | 1.00 | 64.85 | | C |
| ANISOU | 1509 | CE2 | PHE | A | 194 | 7612 | 9319 | 7707 | 2679 | 1956 | 808 | C |
| ATOM | 1510 | CZ | PHE | A | 194 | 49.415 | 59.776 | 17.531 | 1.00 | 63.27 | | C |
| ANISOU | 1510 | CZ | PHE | A | 194 | 7257 | 9407 | 7375 | 3040 | 1952 | 831 | C |
| ATOM | 1511 | N | ARG | A | 195 | 53.528 | 58.997 | 13.075 | 1.00 | 58.61 | | N |
| ANISOU | 1511 | N | ARG | A | 195 | 7000 | 8356 | 6912 | 2665 | 1768 | 1063 | N |
| ATOM | 1512 | CA | ARG | A | 195 | 53.814 | 60.295 | 12.470 | 1.00 | 58.53 | | C |
| ANISOU | 1512 | CA | ARG | A | 195 | 7335 | 8101 | 6802 | 2896 | 1924 | 1175 | C |
| ATOM | 1513 | CB | ARG | A | 195 | 55.326 | 60.607 | 12.508 | 1.00 | 58.65 | | C |
| ANISOU | 1513 | CB | ARG | A | 195 | 7604 | 7776 | 6902 | 2606 | 2008 | 1189 | C |
| ATOM | 1514 | CG | ARG | A | 195 | 55.770 | 61.753 | 11.579 | 1.00 | 66.53 | | C |
| ANISOU | 1514 | CG | ARG | A | 195 | 8956 | 8526 | 7796 | 2786 | 2166 | 1307 | C |
| ATOM | 1515 | CD | ARG | A | 195 | 57.271 | 61.872 | 11.451 | 1.00 | 73.76 | | C |
| ANISOU | 1515 | CD | ARG | A | 195 | 10042 | 9188 | 8796 | 2457 | 2228 | 1303 | C |
| ATOM | 1516 | NE | ARG | A | 195 | 57.865 | 62.311 | 12.708 | 1.00 | 86.83 | | N |
| ANISOU | 1516 | NE | ARG | A | 195 | 11855 | 10609 | 10526 | 2194 | 2326 | 1232 | N |
| ATOM | 1517 | CZ | ARG | A | 195 | 58.084 | 63.579 | 13.026 | 1.00 | 105.03 | | C |
| ANISOU | 1517 | CZ | ARG | A | 195 | 14539 | 12601 | 12765 | 2230 | 2532 | 1265 | C |
| ATOM | 1518 | NH1 | ARG | A | 195 | 57.774 | 64.549 | 12.169 | 1.00 | 97.48 | | N |
| ANISOU | 1518 | NH1 | ARG | A | 195 | 13868 | 11506 | 11662 | 2533 | 2672 | 1380 | N |
| ATOM | 1519 | NH2 | ARG | A | 195 | 58.596 | 63.893 | 14.211 | 1.00 | 85.88 | | N |
| ANISOU | 1519 | NH2 | ARG | A | 195 | 12231 | 9993 | 10405 | 1970 | 2605 | 1182 | N |
| ATOM | 1520 | C | ARG | A | 195 | 53.308 | 60.365 | 11.064 | 1.00 | 63.73 | | C |
| ANISOU | 1520 | C | ARG | A | 195 | 7943 | 8937 | 7333 | 3209 | 1879 | 1256 | C |
| ATOM | 1521 | O | ARG | A | 195 | 52.656 | 61.345 | 10.712 | 1.00 | 64.45 | | O |
| ANISOU | 1521 | O | ARG | A | 195 | 8206 | 9008 | 7274 | 3580 | 1979 | 1335 | O |
| ATOM | 1522 | N | TYR | A | 196 | 53.632 | 59.349 | 10.251 | 1.00 | 60.66 | | N |
| ANISOU | 1522 | N | TYR | A | 196 | 7344 | 8715 | 6989 | 3077 | 1734 | 1237 | N |
| ATOM | 1523 | CA | TYR | A | 196 | 53.291 | 59.314 | 8.842 | 1.00 | 60.39 | | C |
| ANISOU | 1523 | CA | TYR | A | 196 | 7258 | 8851 | 6836 | 3341 | 1677 | 1306 | C |
| ATOM | 1524 | CB | TYR | A | 196 | 54.387 | 58.601 | 8.043 | 1.00 | 61.69 | | C |
| ANISOU | 1524 | CB | TYR | A | 196 | 7394 | 8968 | 7076 | 3103 | 1602 | 1312 | C |
| ATOM | 1525 | CG | TYR | A | 196 | 55.744 | 59.290 | 8.120 | 1.00 | 64.38 | | C |
| ANISOU | 1525 | CG | TYR | A | 196 | 8063 | 8924 | 7476 | 2915 | 1748 | 1363 | C |
| ATOM | 1526 | CD1 | TYR | A | 196 | 56.015 | 60.438 | 7.375 | 1.00 | 66.95 | | C |
| ANISOU | 1526 | CD1 | TYR | A | 196 | 8723 | 9028 | 7686 | 3129 | 1907 | 1481 | C |
| ATOM | 1527 | CE1 | TYR | A | 196 | 57.264 | 61.066 | 7.433 | 1.00 | 67.99 | | C |
| ANISOU | 1527 | CE1 | TYR | A | 196 | 9150 | 8819 | 7864 | 2914 | 2056 | 1513 | C |
| ATOM | 1528 | CZ | TYR | A | 196 | 58.271 | 60.525 | 8.217 | 1.00 | 75.73 | | C |
| ANISOU | 1528 | CZ | TYR | A | 196 | 10058 | 9711 | 9004 | 2503 | 2028 | 1423 | C |
| ATOM | 1529 | OH | TYR | A | 196 | 59.511 | 61.123 | 8.270 | 1.00 | 78.06 | | O |
| ANISOU | 1529 | OH | TYR | A | 196 | 10605 | 9717 | 9338 | 2269 | 2171 | 1435 | O |
| ATOM | 1530 | CE2 | TYR | A | 196 | 58.027 | 59.377 | 8.956 | 1.00 | 66.34 | | C |
| ANISOU | 1530 | CE2 | TYR | A | 196 | 8546 | 8737 | 7922 | 2324 | 1861 | 1314 | C |
| ATOM | 1531 | CD2 | TYR | A | 196 | 56.770 | 58.767 | 8.900 | 1.00 | 65.19 | | C |
| ANISOU | 1531 | CD2 | TYR | A | 196 | 8139 | 8898 | 7733 | 2519 | 1730 | 1287 | C |
| ATOM | 1532 | C | TYR | A | 196 | 51.894 | 58.754 | 8.579 | 1.00 | 65.96 | | C |
| ANISOU | 1532 | C | TYR | A | 196 | 7627 | 9988 | 7448 | 3570 | 1545 | 1257 | C |
| ATOM | 1533 | O | TYR | A | 196 | 51.235 | 59.267 | 7.687 | 1.00 | 66.16 | | O |
| ANISOU | 1533 | O | TYR | A | 196 | 7680 | 10146 | 7310 | 3943 | 1553 | 1328 | O |
| ATOM | 1534 | N | LEU | A | 197 | 51.401 | 57.798 | 9.386 | 1.00 | 64.99 | | N |
| ANISOU | 1534 | N | LEU | A | 197 | 7201 | 10086 | 7408 | 3363 | 1438 | 1136 | N |
| ATOM | 1535 | CA | LEU | A | 197 | 50.039 | 57.237 | 9.259 | 1.00 | 66.24 | | C |
| ANISOU | 1535 | CA | LEU | A | 197 | 7005 | 10682 | 7479 | 3517 | 1322 | 1060 | C |
| ATOM | 1536 | CB | LEU | A | 197 | 49.871 | 55.969 | 10.118 | 1.00 | 66.30 | | C |
| ANISOU | 1536 | CB | LEU | A | 197 | 6735 | 10844 | 7611 | 3150 | 1220 | 921 | C |
| ATOM | 1537 | CG | LEU | A | 197 | 48.859 | 54.923 | 9.650 | 1.00 | 70.44 | | C |
| ANISOU | 1537 | CG | LEU | A | 197 | 6872 | 11811 | 8079 | 3137 | 1065 | 819 | C |
| ATOM | 1538 | CD1 | LEU | A | 197 | 49.346 | 54.196 | 8.413 | 1.00 | 70.30 | | C |
| ANISOU | 1538 | CD1 | LEU | A | 197 | 6805 | 11866 | 8042 | 3100 | 951 | 836 | C |
| ATOM | 1539 | CD2 | LEU | A | 197 | 48.628 | 53.893 | 10.726 | 1.00 | 72.91 | | C |
| ANISOU | 1539 | CD2 | LEU | A | 197 | 6996 | 12204 | 8502 | 2773 | 1017 | 691 | C |
| ATOM | 1540 | C | LEU | A | 197 | 48.916 | 58.252 | 9.577 | 1.00 | 73.15 | | C |
| ANISOU | 1540 | C | LEU | A | 197 | 7910 | 11674 | 8209 | 3912 | 1413 | 1089 | C |
| ATOM | 1541 | O | LEU | A | 197 | 48.070 | 58.493 | 8.714 | 1.00 | 73.27 | | O |
| ANISOU | 1541 | O | LEU | A | 197 | 7812 | 11967 | 8061 | 4266 | 1369 | 1116 | O |
| ATOM | 1542 | N | HIS | A | 198 | 48.919 | 58.847 | 10.790 | 1.00 | 71.88 | | N |
| ANISOU | 1542 | N | HIS | A | 198 | 7908 | 11311 | 8092 | 3874 | 1539 | 1082 | N |
| ATOM | 1543 | CA | HIS | A | 198 | 47.943 | 59.860 | 11.240 | 1.00 | 73.48 | | C |
| ANISOU | 1543 | CA | HIS | A | 198 | 8182 | 11585 | 8153 | 4258 | 1646 | 1112 | C |
| ATOM | 1544 | CB | HIS | A | 198 | 47.201 | 59.382 | 12.518 | 1.00 | 74.75 | | C |
| ANISOU | 1544 | CB | HIS | A | 198 | 8063 | 11988 | 8352 | 4151 | 1620 | 989 | C |
| ATOM | 1545 | CG | HIS | A | 198 | 46.925 | 57.912 | 12.566 | 1.00 | 78.36 | | C |
| ANISOU | 1545 | CG | HIS | A | 198 | 8123 | 12756 | 8895 | 3831 | 1455 | 862 | C |
| ATOM | 1546 | ND1 | HIS | A | 198 | 46.344 | 57.259 | 11.500 | 1.00 | 80.45 | | N |
| ANISOU | 1546 | ND1 | HIS | A | 198 | 8090 | 13410 | 9069 | 3938 | 1314 | 824 | N |
| ATOM | 1547 | CE1 | HIS | A | 198 | 46.221 | 56.001 | 11.879 | 1.00 | 79.89 | | C |
| ANISOU | 1547 | CE1 | HIS | A | 198 | 7755 | 13498 | 9100 | 3565 | 1209 | 703 | C |
| ATOM | 1548 | NE2 | HIS | A | 198 | 46.732 | 55.803 | 13.094 | 1.00 | 79.96 | | N |
| ANISOU | 1548 | NE2 | HIS | A | 198 | 7870 | 13260 | 9251 | 3257 | 1272 | 667 | N |
| ATOM | 1549 | CD2 | HIS | A | 198 | 47.172 | 57.021 | 13.549 | 1.00 | 79.98 | | C |
| ANISOU | 1549 | CD2 | HIS | A | 198 | 8219 | 12921 | 9250 | 3418 | 1420 | 764 | C |
| ATOM | 1550 | C | HIS | A | 198 | 48.659 | 61.215 | 11.522 | 1.00 | 79.59 | | C |
| ANISOU | 1550 | C | HIS | A | 198 | 9443 | 11883 | 8913 | 4350 | 1849 | 1217 | C |
| ATOM | 1551 | O | HIS | A | 198 | 48.834 | 61.551 | 12.710 | 1.00 | 79.20 | | O |
| ANISOU | 1551 | O | HIS | A | 198 | 9509 | 11646 | 8939 | 4197 | 1940 | 1179 | O |
| ATOM | 1552 | N | PRO | A | 199 | 49.105 | 62.006 | 10.493 | 1.00 | 77.12 | | N |
| ANISOU | 1552 | N | PRO | A | 199 | 9439 | 11365 | 8498 | 4579 | 1931 | 1342 | N |
| ATOM | 1553 | CA | PRO | A | 199 | 49.861 | 63.232 | 10.811 | 1.00 | 77.49 | | C |
| ANISOU | 1553 | CA | PRO | A | 199 | 9979 | 10936 | 8530 | 4605 | 2142 | 1429 | C |
| ATOM | 1554 | CB | PRO | A | 199 | 50.357 | 63.728 | 9.456 | 1.00 | 78.74 | | C |
| ANISOU | 1554 | CB | PRO | A | 199 | 10386 | 10950 | 8581 | 4795 | 2190 | 1553 | C |
| ATOM | 1555 | CG | PRO | A | 199 | 49.468 | 63.101 | 8.473 | 1.00 | 83.05 | | C |
| ANISOU | 1555 | CG | PRO | A | 199 | 10600 | 11939 | 9017 | 5060 | 2026 | 1549 | C |
| ATOM | 1556 | CD | PRO | A | 199 | 49.037 | 61.790 | 9.035 | 1.00 | 78.55 | | C |
| ANISOU | 1556 | CD | PRO | A | 199 | 9554 | 11717 | 8574 | 4781 | 1844 | 1404 | C |
| ATOM | 1557 | C | PRO | A | 199 | 49.080 | 64.286 | 11.584 | 1.00 | 84.63 | | C |
| ANISOU | 1557 | C | PRO | A | 199 | 11077 | 11765 | 9313 | 4919 | 2290 | 1451 | C |
| ATOM | 1558 | O | PRO | A | 199 | 49.652 | 64.879 | 12.504 | 1.00 | 84.78 | | O |
| ANISOU | 1558 | O | PRO | A | 199 | 11387 | 11432 | 9393 | 4751 | 2433 | 1447 | O |
| ATOM | 1559 | N | GLU | A | 200 | 47.772 | 64.455 | 11.285 | 1.00 | 82.59 | | N |
| ANISOU | 1559 | N | GLU | A | 200 | 10641 | 11859 | 8881 | 5361 | 2252 | 1461 | N |
| ATOM | 1560 | CA | GLU | A | 200 | 46.911 | 65.426 | 11.970 | 1.00 | 83.32 | | C |
| ANISOU | 1560 | CA | GLU | A | 200 | 10893 | 11934 | 8832 | 5725 | 2387 | 1482 | C |
| ATOM | 1561 | CB | GLU | A | 200 | 45.528 | 65.510 | 11.308 | 1.00 | 85.05 | | C |
| ANISOU | 1561 | CB | GLU | A | 200 | 10863 | 12620 | 8831 | 6253 | 2313 | 1497 | C |
| ATOM | 1562 | CG | GLU | A | 200 | 45.505 | 66.285 | 9.991 | 1.00 | 100.67 | | C |
| ANISOU | 1562 | CG | GLU | A | 200 | 13117 | 14531 | 10599 | 6697 | 2366 | 1640 | C |
| ATOM | 1563 | CD | GLU | A | 200 | 46.222 | 65.682 | 8.790 | 1.00 | 132.37 | | C |
| ANISOU | 1563 | CD | GLU | A | 200 | 17075 | 18560 | 14659 | 6538 | 2256 | 1675 | C |
| ATOM | 1564 | OE1 | GLU | A | 200 | 46.344 | 64.437 | 8.723 | 1.00 | 139.19 | | O |
| ANISOU | 1564 | OE1 | GLU | A | 200 | 17527 | 19693 | 15666 | 6202 | 2072 | 1572 | O |
| ATOM | 1565 | OE2 | GLU | A | 200 | 46.663 | 66.461 | 7.912 | 1.00 | 125.87 | | O |
| ANISOU | 1565 | OE2 | GLU | A | 200 | 16640 | 17467 | 13717 | 6752 | 2363 | 1807 | O |
| ATOM | 1566 | C | GLU | A | 200 | 46.798 | 65.125 | 13.464 | 1.00 | 88.68 | | C |
| ANISOU | 1566 | C | GLU | A | 200 | 11452 | 12595 | 9646 | 5436 | 2404 | 1370 | C |
| ATOM | 1567 | O | GLU | A | 200 | 46.741 | 66.056 | 14.273 | 1.00 | 88.48 | | O |
| ANISOU | 1567 | O | GLU | A | 200 | 11736 | 12306 | 9575 | 5542 | 2572 | 1392 | O |
| ATOM | 1568 | N | ALA | A | 201 | 46.822 | 63.822 | 13.827 | 1.00 | 85.69 | | N |
| ANISOU | 1568 | N | ALA | A | 201 | 10661 | 12468 | 9430 | 5058 | 2240 | 1250 | N |
| ATOM | 1569 | CA | ALA | A | 201 | 46.776 | 63.356 | 15.216 | 1.00 | 85.29 | | C |
| ANISOU | 1569 | CA | ALA | A | 201 | 10474 | 12419 | 9515 | 4741 | 2238 | 1139 | C |
| ATOM | 1570 | CB | ALA | A | 201 | 46.360 | 61.892 | 15.268 | 1.00 | 86.26 | | C |
| ANISOU | 1570 | CB | ALA | A | 201 | 10102 | 12937 | 9738 | 4467 | 2046 | 1020 | C |
| ATOM | 1571 | C | ALA | A | 201 | 48.141 | 63.526 | 15.867 | 1.00 | 86.61 | | C |
| ANISOU | 1571 | C | ALA | A | 201 | 10960 | 12113 | 9834 | 4349 | 2324 | 1141 | C |
| ATOM | 1572 | O | ALA | A | 201 | 48.224 | 63.938 | 17.027 | 1.00 | 85.27 | | O |
| ANISOU | 1572 | O | ALA | A | 201 | 10972 | 11737 | 9691 | 4274 | 2438 | 1111 | O |
| ATOM | 1573 | N | PHE | A | 202 | 49.208 | 63.207 | 15.111 | 1.00 | 82.10 | | N |
| ANISOU | 1573 | N | PHE | A | 202 | 10451 | 11392 | 9350 | 4108 | 2270 | 1170 | N |
| ATOM | 1574 | CA | PHE | A | 202 | 50.581 | 63.308 | 15.584 | 1.00 | 81.47 | | C |
| ANISOU | 1574 | CA | PHE | A | 202 | 10628 | 10921 | 9407 | 3724 | 2334 | 1161 | C |
| ATOM | 1575 | CB | PHE | A | 202 | 51.560 | 62.660 | 14.589 | 1.00 | 82.88 | | C |
| ANISOU | 1575 | CB | PHE | A | 202 | 10754 | 11074 | 9665 | 3505 | 2237 | 1184 | C |
| ATOM | 1576 | CG | PHE | A | 202 | 53.007 | 62.709 | 15.024 | 1.00 | 83.88 | | C |
| ANISOU | 1576 | CG | PHE | A | 202 | 11091 | 10858 | 9923 | 3104 | 2291 | 1161 | C |
| ATOM | 1577 | CD1 | PHE | A | 202 | 53.497 | 61.815 | 15.964 | 1.00 | 86.22 | | C |
| ANISOU | 1577 | CD1 | PHE | A | 202 | 11212 | 11179 | 10370 | 2722 | 2202 | 1053 | C |
| ATOM | 1578 | CE1 | PHE | A | 202 | 54.830 | 61.867 | 16.367 | 1.00 | 87.04 | | C |
| ANISOU | 1578 | CE1 | PHE | A | 202 | 11484 | 11011 | 10576 | 2373 | 2243 | 1021 | C |
| ATOM | 1579 | CZ | PHE | A | 202 | 55.680 | 62.802 | 15.822 | 1.00 | 86.27 | | C |
| ANISOU | 1579 | CZ | PHE | A | 202 | 11722 | 10615 | 10440 | 2366 | 2382 | 1088 | C |
| ATOM | 1580 | CE2 | PHE | A | 202 | 55.212 | 63.697 | 14.892 | 1.00 | 88.22 | | C |
| ANISOU | 1580 | CE2 | PHE | A | 202 | 12180 | 10799 | 10542 | 2722 | 2488 | 1200 | C |
| ATOM | 1581 | CD2 | PHE | A | 202 | 53.881 | 63.644 | 14.482 | 1.00 | 85.73 | | C |
| ANISOU | 1581 | CD2 | PHE | A | 202 | 11701 | 10757 | 10115 | 3109 | 2436 | 1241 | C |
| ATOM | 1582 | C | PHE | A | 202 | 50.967 | 64.753 | 15.896 | 1.00 | 85.36 | | C |
| ANISOU | 1582 | C | PHE | A | 202 | 11617 | 10997 | 9817 | 3856 | 2558 | 1231 | C |
| ATOM | 1583 | O | PHE | A | 202 | 51.557 | 65.001 | 16.945 | 1.00 | 85.07 | | O |
| ANISOU | 1583 | O | PHE | A | 202 | 11748 | 10713 | 9862 | 3598 | 2638 | 1177 | O |
| ATOM | 1584 | N | ALA | A | 203 | 50.626 | 65.697 | 15.001 | 1.00 | 81.64 | | N |
| ANISOU | 1584 | N | ALA | A | 203 | 11396 | 10453 | 9173 | 4259 | 2661 | 1346 | N |
| ATOM | 1585 | CA | ALA | A | 203 | 50.943 | 67.113 | 15.167 | 1.00 | 81.52 | | C |
| ANISOU | 1585 | CA | ALA | A | 203 | 11909 | 10020 | 9046 | 4418 | 2896 | 1422 | C |
| ATOM | 1586 | CB | ALA | A | 203 | 50.587 | 67.878 | 13.911 | 1.00 | 82.55 | | C |
| ANISOU | 1586 | CB | ALA | A | 203 | 12264 | 10127 | 8976 | 4871 | 2972 | 1559 | C |
| ATOM | 1587 | C | ALA | A | 203 | 50.242 | 67.728 | 16.358 | 1.00 | 84.98 | | C |
| ANISOU | 1587 | C | ALA | A | 203 | 12463 | 10401 | 9425 | 4567 | 3005 | 1384 | C |
| ATOM | 1588 | O | ALA | A | 203 | 50.833 | 68.588 | 17.013 | 1.00 | 84.74 | | O |
| ANISOU | 1588 | O | ALA | A | 203 | 12830 | 9977 | 9390 | 4451 | 3179 | 1384 | O |
| ATOM | 1589 | N | SER | A | 204 | 48.988 | 67.291 | 16.639 | 1.00 | 81.17 | | N |
| ANISOU | 1589 | N | SER | A | 204 | 11633 | 10318 | 8888 | 4815 | 2910 | 1345 | N |
| ATOM | 1590 | CA | SER | A | 204 | 48.174 | 67.771 | 17.760 | 1.00 | 80.77 | | C |
| ANISOU | 1590 | CA | SER | A | 204 | 11628 | 10290 | 8772 | 4994 | 3002 | 1303 | C |
| ATOM | 1591 | CB | SER | A | 204 | 46.718 | 67.331 | 17.604 | 1.00 | 84.43 | | C |
| ANISOU | 1591 | CB | SER | A | 204 | 11680 | 11272 | 9128 | 5357 | 2895 | 1281 | C |
| ATOM | 1592 | OG | SER | A | 204 | 45.931 | 67.618 | 18.754 | 1.00 | 91.11 | | O |
| ANISOU | 1592 | OG | SER | A | 204 | 12509 | 12188 | 9919 | 5502 | 2973 | 1227 | O |
| ATOM | 1593 | C | SER | A | 204 | 48.722 | 67.283 | 19.095 | 1.00 | 83.65 | | C |
| ANISOU | 1593 | C | SER | A | 204 | 11926 | 10542 | 9315 | 4530 | 2987 | 1185 | C |
| ATOM | 1594 | O | SER | A | 204 | 48.834 | 68.068 | 20.035 | 1.00 | 83.74 | | O |
| ANISOU | 1594 | O | SER | A | 204 | 12243 | 10278 | 9298 | 4524 | 3140 | 1169 | O |
| ATOM | 1595 | N | LEU | A | 205 | 49.034 | 65.986 | 19.182 | 1.00 | 78.81 | | N |
| ANISOU | 1595 | N | LEU | A | 205 | 10934 | 10140 | 8871 | 4159 | 2806 | 1103 | N |
| ATOM | 1596 | CA | LEU | A | 205 | 49.539 | 65.367 | 20.395 | 1.00 | 77.92 | | C |
| ANISOU | 1596 | CA | LEU | A | 205 | 10729 | 9961 | 8917 | 3731 | 2767 | 992 | C |
| ATOM | 1597 | CB | LEU | A | 205 | 49.459 | 63.835 | 20.269 | 1.00 | 77.54 | | C |
| ANISOU | 1597 | CB | LEU | A | 205 | 10209 | 10256 | 8997 | 3464 | 2555 | 916 | C |
| ATOM | 1598 | CG | LEU | A | 205 | 49.975 | 62.974 | 21.415 | 1.00 | 81.29 | | C |
| ANISOU | 1598 | CG | LEU | A | 205 | 10556 | 10707 | 9624 | 3034 | 2491 | 805 | C |
| ATOM | 1599 | CD1 | LEU | A | 205 | 49.302 | 63.319 | 22.715 | 1.00 | 81.47 | | C |
| ANISOU | 1599 | CD1 | LEU | A | 205 | 10613 | 10735 | 9607 | 3106 | 2583 | 749 | C |
| ATOM | 1600 | CD2 | LEU | A | 205 | 49.775 | 61.509 | 21.109 | 1.00 | 82.42 | | C |
| ANISOU | 1600 | CD2 | LEU | A | 205 | 10279 | 11180 | 9855 | 2833 | 2299 | 746 | C |
| ATOM | 1601 | C | LEU | A | 205 | 50.943 | 65.854 | 20.728 | 1.00 | 82.38 | | C |
| ANISOU | 1601 | C | LEU | A | 205 | 11655 | 10084 | 9561 | 3403 | 2863 | 986 | C |
| ATOM | 1602 | O | LEU | A | 205 | 51.203 | 66.156 | 21.894 | 1.00 | 81.97 | | O |
| ANISOU | 1602 | O | LEU | A | 205 | 11751 | 9846 | 9546 | 3225 | 2938 | 920 | O |
| ATOM | 1603 | N | SER | A | 206 | 51.828 | 65.968 | 19.716 | 1.00 | 79.77 | | N |
| ANISOU | 1603 | N | SER | A | 206 | 11464 | 9599 | 9245 | 3321 | 2865 | 1048 | N |
| ATOM | 1604 | CA | SER | A | 206 | 53.205 | 66.435 | 19.909 | 1.00 | 80.59 | | C |
| ANISOU | 1604 | CA | SER | A | 206 | 11885 | 9320 | 9416 | 2988 | 2960 | 1033 | C |
| ATOM | 1605 | C | SER | A | 206 | 53.254 | 67.867 | 20.467 | 1.00 | 86.67 | | C |
| ANISOU | 1605 | C | SER | A | 206 | 13149 | 9708 | 10073 | 3104 | 3197 | 1055 | C |
| ATOM | 1606 | O | SER | A | 206 | 53.944 | 68.102 | 21.462 | 1.00 | 86.26 | | O |
| ANISOU | 1606 | O | SER | A | 206 | 13267 | 9423 | 10085 | 2791 | 3262 | 973 | O |
| ATOM | 1607 | CB | SER | A | 206 | 54.021 | 66.320 | 18.621 | 1.00 | 84.30 | | C |
| ANISOU | 1607 | CB | SER | A | 206 | 12391 | 9736 | 9903 | 2913 | 2930 | 1098 | C |
| ATOM | 1608 | OG | SER | A | 206 | 54.603 | 65.036 | 18.453 | 1.00 | 92.42 | | O |
| ANISOU | 1608 | OG | SER | A | 206 | 13079 | 10956 | 11082 | 2589 | 2742 | 1037 | O |
| ATOM | 1609 | N | ALA | A | 207 | 52.483 | 68.801 | 19.859 | 1.00 | 84.30 | | N |
| ANISOU | 1609 | N | ALA | A | 207 | 13085 | 9353 | 9591 | 3565 | 3324 | 1159 | N |
| ATOM | 1610 | CA | ALA | A | 207 | 52.413 | 70.206 | 20.280 | 1.00 | 84.44 | | C |
| ANISOU | 1610 | CA | ALA | A | 207 | 13622 | 8993 | 9467 | 3741 | 3568 | 1194 | C |
| ATOM | 1611 | CB | ALA | A | 207 | 51.514 | 70.988 | 19.345 | 1.00 | 85.13 | | C |
| ANISOU | 1611 | CB | ALA | A | 207 | 13916 | 9089 | 9341 | 4300 | 3669 | 1329 | C |
| ATOM | 1612 | C | ALA | A | 207 | 51.916 | 70.342 | 21.717 | 1.00 | 89.54 | | C |
| ANISOU | 1612 | C | ALA | A | 207 | 14268 | 9636 | 10118 | 3725 | 3607 | 1105 | C |
| ATOM | 1613 | O | ALA | A | 207 | 52.392 | 71.212 | 22.446 | 1.00 | 90.25 | | O |
| ANISOU | 1613 | O | ALA | A | 207 | 14757 | 9365 | 10169 | 3612 | 3780 | 1072 | O |
| ATOM | 1614 | N | ARG | A | 208 | 50.981 | 69.466 | 22.126 | 1.00 | 85.49 | | N |
| ANISOU | 1614 | N | ARG | A | 208 | 13310 | 9525 | 9647 | 3815 | 3454 | 1059 | N |
| ATOM | 1615 | CA | ARG | A | 208 | 50.428 | 69.445 | 23.469 | 1.00 | 84.97 | | C |
| ANISOU | 1615 | CA | ARG | A | 208 | 13178 | 9517 | 9588 | 3799 | 3474 | 973 | C |
| ATOM | 1616 | CB | ARG | A | 208 | 49.197 | 68.535 | 23.517 | 1.00 | 83.35 | | C |
| ANISOU | 1616 | CB | ARG | A | 208 | 12467 | 9813 | 9387 | 3988 | 3319 | 950 | C |
| ATOM | 1617 | C | ARG | A | 208 | 51.512 | 69.007 | 24.463 | 1.00 | 90.77 | | C |
| ANISOU | 1617 | C | ARG | A | 208 | 13917 | 10085 | 10484 | 3265 | 3438 | 856 | C |
| ATOM | 1618 | O | ARG | A | 208 | 51.621 | 69.588 | 25.539 | 1.00 | 90.33 | | O |
| ANISOU | 1618 | O | ARG | A | 208 | 14109 | 9808 | 10404 | 3184 | 3555 | 797 | O |
| ATOM | 1619 | N | ILE | A | 209 | 52.347 | 68.025 | 24.080 | 1.00 | 88.91 | | N |
| ANISOU | 1619 | N | ILE | A | 209 | 13433 | 9951 | 10399 | 2916 | 3280 | 822 | N |
| ATOM | 1620 | CA | ILE | A | 209 | 53.450 | 67.548 | 24.926 | 1.00 | 89.18 | | C |
| ANISOU | 1620 | CA | ILE | A | 209 | 13445 | 9867 | 10572 | 2429 | 3225 | 712 | C |
| ATOM | 1621 | C | ILE | A | 209 | 54.584 | 68.596 | 24.948 | 1.00 | 95.18 | | C |
| ANISOU | 1621 | C | ILE | A | 209 | 14662 | 10198 | 11305 | 2230 | 3391 | 703 | C |
| ATOM | 1622 | O | ILE | A | 209 | 55.183 | 68.806 | 26.003 | 1.00 | 95.14 | | O |
| ANISOU | 1622 | O | ILE | A | 209 | 14794 | 10016 | 11336 | 1944 | 3435 | 602 | O |
| ATOM | 1623 | CB | ILE | A | 209 | 53.916 | 66.110 | 24.538 | 1.00 | 91.83 | | C |
| ANISOU | 1623 | CB | ILE | A | 209 | 13364 | 10474 | 11055 | 2162 | 3002 | 678 | C |
| ATOM | 1624 | CG1 | ILE | A | 209 | 52.770 | 65.101 | 24.717 | 1.00 | 91.86 | | C |
| ANISOU | 1624 | CG1 | ILE | A | 209 | 12950 | 10882 | 11072 | 2321 | 2863 | 670 | C |
| ATOM | 1625 | CG2 | ILE | A | 209 | 55.140 | 65.664 | 25.346 | 1.00 | 92.47 | | C |
| ANISOU | 1625 | CG2 | ILE | A | 209 | 13426 | 10452 | 11255 | 1702 | 2940 | 565 | C |
| ATOM | 1626 | CD1 | ILE | A | 209 | 52.880 | 63.877 | 23.886 | 1.00 | 96.38 | | C |
| ANISOU | 1626 | CD1 | ILE | A | 209 | 13168 | 11720 | 11731 | 2204 | 2676 | 675 | C |
| ATOM | 1627 | N | GLN | A | 210 | 54.818 | 69.302 | 23.810 | 1.00 | 92.83 | | N |
| ANISOU | 1627 | N | GLN | A | 210 | 14613 | 9733 | 10926 | 2385 | 3495 | 803 | N |
| ATOM | 1628 | CA | GLN | A | 210 | 55.834 | 70.358 | 23.663 | 1.00 | 93.05 | | C |
| ANISOU | 1628 | CA | GLN | A | 210 | 15103 | 9345 | 10906 | 2200 | 3683 | 801 | C |
| ATOM | 1629 | CB | GLN | A | 210 | 56.033 | 70.744 | 22.185 | 1.00 | 94.19 | | C |
| ANISOU | 1629 | CB | GLN | A | 210 | 15402 | 9405 | 10980 | 2364 | 3747 | 924 | C |
| ATOM | 1630 | C | GLN | A | 210 | 55.543 | 71.594 | 24.544 | 1.00 | 98.06 | | C |
| ANISOU | 1630 | C | GLN | A | 210 | 16193 | 9654 | 11411 | 2316 | 3905 | 779 | C |
| ATOM | 1631 | O | GLN | A | 210 | 56.370 | 72.510 | 24.616 | 1.00 | 97.86 | | O |
| ANISOU | 1631 | O | GLN | A | 210 | 16592 | 9257 | 11334 | 2125 | 4085 | 755 | O |
| ATOM | 1632 | N | ALA | A | 211 | 54.376 | 71.591 | 25.230 | 1.00 | 95.30 | | N |
| ANISOU | 1632 | N | ALA | A | 211 | 15752 | 9453 | 11003 | 2611 | 3898 | 779 | N |
| ATOM | 1633 | CA | ALA | A | 211 | 53.928 | 72.630 | 26.156 | 1.00 | 95.65 | | C |
| ANISOU | 1633 | CA | ALA | A | 211 | 16182 | 9242 | 10920 | 2769 | 4089 | 755 | C |
| ATOM | 1634 | CB | ALA | A | 211 | 52.411 | 72.732 | 26.132 | 1.00 | 96.35 | | C |
| ANISOU | 1634 | CB | ALA | A | 211 | 16164 | 9556 | 10887 | 3307 | 4097 | 830 | C |
| ATOM | 1635 | C | ALA | A | 211 | 54.432 | 72.344 | 27.589 | 1.00 | 101.06 | | C |
| ANISOU | 1635 | C | ALA | A | 211 | 16817 | 9881 | 11699 | 2380 | 4053 | 600 | C |
| ATOM | 1636 | O | ALA | A | 211 | 55.001 | 73.236 | 28.223 | 1.00 | 101.59 | | O |
| ANISOU | 1636 | O | ALA | A | 211 | 17295 | 9597 | 11710 | 2205 | 4219 | 535 | O |
| ATOM | 1637 | N | THR | A | 212 | 54.242 | 71.106 | 28.088 | 1.00 | 97.21 | | N |
| ANISOU | 1637 | N | THR | A | 212 | 15849 | 9743 | 11344 | 2236 | 3840 | 538 | N |
| ATOM | 1638 | CA | THR | A | 212 | 54.730 | 70.694 | 29.406 | 1.00 | 96.72 | | C |
| ANISOU | 1638 | CA | THR | A | 212 | 15701 | 9681 | 11366 | 1883 | 3779 | 397 | C |
| ATOM | 1639 | CB | THR | A | 212 | 53.928 | 69.498 | 29.943 | 1.00 | 103.19 | | C |
| ANISOU | 1639 | CB | THR | A | 212 | 16055 | 10896 | 12258 | 1945 | 3601 | 372 | C |
| ATOM | 1640 | OG1 | THR | A | 212 | 53.962 | 68.431 | 28.992 | 1.00 | 101.41 | | O |
| ANISOU | 1640 | OG1 | THR | A | 212 | 15440 | 10965 | 12125 | 1930 | 3422 | 421 | O |
| ATOM | 1641 | CG2 | THR | A | 212 | 52.493 | 69.856 | 30.287 | 1.00 | 101.73 | | C |
| ANISOU | 1641 | CG2 | THR | A | 212 | 15887 | 10813 | 11953 | 2377 | 3688 | 417 | C |
| ATOM | 1642 | C | THR | A | 212 | 56.225 | 70.349 | 29.326 | 1.00 | 100.74 | | C |
| ANISOU | 1642 | C | THR | A | 212 | 16181 | 10105 | 11989 | 1395 | 3705 | 314 | C |
| ATOM | 1643 | O | THR | A | 212 | 56.900 | 70.378 | 30.350 | 1.00 | 100.88 | | O |
| ANISOU | 1643 | O | THR | A | 212 | 16271 | 10021 | 12038 | 1079 | 3705 | 189 | O |
| ATOM | 1644 | N | GLN | A | 213 | 56.725 | 70.030 | 28.103 | 1.00 | 96.87 | | N |
| ANISOU | 1644 | N | GLN | A | 213 | 15581 | 9677 | 11548 | 1351 | 3643 | 381 | N |
| ATOM | 1645 | CA | GLN | A | 213 | 58.089 | 69.622 | 27.721 | 1.00 | 96.53 | | C |
| ANISOU | 1645 | CA | GLN | A | 213 | 15454 | 9617 | 11605 | 948 | 3565 | 324 | C |
| ATOM | 1646 | CB | GLN | A | 213 | 58.377 | 69.955 | 26.249 | 1.00 | 97.80 | | C |
| ANISOU | 1646 | CB | GLN | A | 213 | 15721 | 9702 | 11737 | 1047 | 3625 | 435 | C |
| ATOM | 1647 | C | GLN | A | 213 | 59.227 | 70.165 | 28.590 | 1.00 | 100.10 | | C |
| ANISOU | 1647 | C | GLN | A | 213 | 16154 | 9821 | 12058 | 539 | 3650 | 183 | C |
| ATOM | 1648 | O | GLN | A | 213 | 59.955 | 69.359 | 29.177 | 1.00 | 99.86 | | O |
| ANISOU | 1648 | O | GLN | A | 213 | 15877 | 9943 | 12124 | 223 | 3501 | 74 | O |
| ATOM | 1649 | N | GLU | A | 214 | 59.399 | 71.506 | 28.652 | 1.00 | 96.03 | | N |
| ANISOU | 1649 | N | GLU | A | 214 | 16130 | 8934 | 11424 | 544 | 3889 | 178 | N |
| ATOM | 1650 | CA | GLU | A | 214 | 60.471 | 72.151 | 29.424 | 1.00 | 95.58 | | C |
| ANISOU | 1650 | CA | GLU | A | 214 | 16346 | 8624 | 11345 | 136 | 3995 | 29 | C |
| ATOM | 1651 | CB | GLU | A | 214 | 60.529 | 73.682 | 29.178 | 1.00 | 97.59 | | C |
| ANISOU | 1651 | CB | GLU | A | 214 | 17198 | 8435 | 11446 | 192 | 4292 | 55 | C |
| ATOM | 1652 | CG | GLU | A | 214 | 59.366 | 74.512 | 29.728 | 1.00 | 113.58 | | C |
| ANISOU | 1652 | CG | GLU | A | 214 | 19553 | 10272 | 13332 | 570 | 4451 | 104 | C |
| ATOM | 1653 | CD | GLU | A | 214 | 58.228 | 74.863 | 28.782 | 1.00 | 144.43 | | C |
| ANISOU | 1653 | CD | GLU | A | 214 | 23562 | 14177 | 17139 | 1118 | 4527 | 288 | C |
| ATOM | 1654 | OE1 | GLU | A | 214 | 58.122 | 74.245 | 27.696 | 1.00 | 147.88 | | O |
| ANISOU | 1654 | OE1 | GLU | A | 214 | 23727 | 14829 | 17630 | 1253 | 4414 | 392 | O |
| ATOM | 1655 | OE2 | GLU | A | 214 | 57.434 | 75.765 | 29.138 | 1.00 | 137.25 | | O |
| ANISOU | 1655 | OE2 | GLU | A | 214 | 23007 | 13059 | 16083 | 1428 | 4700 | 326 | O |
| ATOM | 1656 | C | GLU | A | 214 | 60.428 | 71.811 | 30.936 | 1.00 | 95.90 | | C |
| ANISOU | 1656 | C | GLU | A | 214 | 16283 | 8745 | 11408 | −16 | 3914 | −107 | C |
| ATOM | 1657 | O | GLU | A | 214 | 61.480 | 71.552 | 31.517 | 1.00 | 95.48 | | O |
| ANISOU | 1657 | O | GLU | A | 214 | 16157 | 8718 | 11405 | −417 | 3848 | −249 | O |
| ATOM | 1658 | N | ALA | A | 215 | 59.221 | 71.769 | 31.544 | 1.00 | 89.39 | | N |
| ANISOU | 1658 | N | ALA | A | 215 | 15428 | 7994 | 10542 | 311 | 3912 | −64 | N |
| ATOM | 1659 | CA | ALA | A | 215 | 59.004 | 71.455 | 32.960 | 1.00 | 87.70 | | C |
| ANISOU | 1659 | CA | ALA | A | 215 | 15131 | 7857 | 10333 | 226 | 3850 | −174 | C |
| ATOM | 1660 | CB | ALA | A | 215 | 57.567 | 71.776 | 33.351 | 1.00 | 88.39 | | C |
| ANISOU | 1660 | CB | ALA | A | 215 | 15306 | 7947 | 10331 | 656 | 3934 | −100 | C |
| ATOM | 1661 | C | ALA | A | 215 | 59.345 | 69.991 | 33.302 | 1.00 | 88.24 | | C |
| ANISOU | 1661 | C | ALA | A | 215 | 14708 | 8284 | 10535 | 34 | 3589 | −230 | C |
| ATOM | 1662 | O | ALA | A | 215 | 59.955 | 69.737 | 34.344 | 1.00 | 87.76 | | O |
| ANISOU | 1662 | O | ALA | A | 215 | 14608 | 8247 | 10490 | −244 | 3524 | −366 | O |
| ATOM | 1663 | N | ARG | A | 216 | 58.950 | 69.039 | 32.428 | 1.00 | 82.32 | | N |
| ANISOU | 1663 | N | ARG | A | 216 | 13605 | 7809 | 9864 | 193 | 3444 | −128 | N |
| ATOM | 1664 | CA | ARG | A | 216 | 59.222 | 67.607 | 32.573 | 1.00 | 80.77 | | C |
| ANISOU | 1664 | CA | ARG | A | 216 | 12971 | 7937 | 9781 | 51 | 3208 | −159 | C |
| ATOM | 1665 | CB | ARG | A | 216 | 58.551 | 66.809 | 31.448 | 1.00 | 79.44 | | C |
| ANISOU | 1665 | CB | ARG | A | 216 | 12506 | 8011 | 9666 | 295 | 3104 | −28 | C |
| ATOM | 1666 | CG | ARG | A | 216 | 57.141 | 66.361 | 31.769 | 1.00 | 86.80 | | C |
| ANISOU | 1666 | CG | ARG | A | 216 | 13263 | 9139 | 10578 | 605 | 3076 | 28 | C |
| ATOM | 1667 | CD | ARG | A | 216 | 56.662 | 65.346 | 30.752 | 1.00 | 91.15 | | C |
| ANISOU | 1667 | CD | ARG | A | 216 | 13461 | 9981 | 11192 | 746 | 2934 | 117 | C |
| ATOM | 1668 | NE | ARG | A | 216 | 56.782 | 63.965 | 31.229 | 1.00 | 91.84 | | N |
| ANISOU | 1668 | NE | ARG | A | 216 | 13214 | 10320 | 11363 | 573 | 2747 | 63 | N |
| ATOM | 1669 | CZ | ARG | A | 216 | 57.842 | 63.185 | 31.031 | 1.00 | 105.42 | | C |
| ANISOU | 1669 | CZ | ARG | A | 216 | 14786 | 12110 | 13161 | 307 | 2610 | 20 | C |
| ATOM | 1670 | NH1 | ARG | A | 216 | 58.904 | 63.644 | 30.376 | 1.00 | 93.49 | | N |
| ANISOU | 1670 | NH1 | ARG | A | 216 | 13398 | 10459 | 11664 | 155 | 2636 | 16 | N |
| ATOM | 1671 | NH2 | ARG | A | 216 | 57.851 | 61.938 | 31.490 | 1.00 | 89.78 | | N |
| ANISOU | 1671 | NH2 | ARG | A | 216 | 12543 | 10339 | 11230 | 194 | 2456 | −20 | N |
| ATOM | 1672 | C | ARG | A | 216 | 60.723 | 67.363 | 32.544 | 1.00 | 82.94 | | C |
| ANISOU | 1672 | C | ARG | A | 216 | 13192 | 8209 | 10111 | −352 | 3127 | −263 | C |
| ATOM | 1673 | O | ARG | A | 216 | 61.244 | 66.704 | 33.443 | 1.00 | 82.13 | | O |
| ANISOU | 1673 | O | ARG | A | 216 | 12930 | 8236 | 10041 | −568 | 3001 | −370 | O |
| ATOM | 1674 | N | GLU | A | 217 | 61.420 | 67.941 | 31.534 | 1.00 | 78.87 | | N |
| ANISOU | 1674 | N | GLU | A | 217 | 12823 | 7553 | 9592 | −446 | 3212 | −234 | N |
| ATOM | 1675 | CA | GLU | A | 217 | 62.867 | 67.835 | 31.339 | 1.00 | 78.23 | | C |
| ANISOU | 1675 | CA | GLU | A | 217 | 12691 | 7483 | 9551 | −823 | 3162 | −332 | C |
| ATOM | 1676 | CB | GLU | A | 217 | 63.295 | 68.424 | 29.988 | 1.00 | 79.53 | | C |
| ANISOU | 1676 | CB | GLU | A | 217 | 13002 | 7511 | 9706 | −831 | 3276 | −255 | C |
| ATOM | 1677 | C | GLU | A | 217 | 63.679 | 68.428 | 32.501 | 1.00 | 81.30 | | C |
| ANISOU | 1677 | C | GLU | A | 217 | 13272 | 7732 | 9887 | −1150 | 3221 | −509 | C |
| ATOM | 1678 | O | GLU | A | 217 | 64.809 | 67.984 | 32.705 | 1.00 | 81.01 | | O |
| ANISOU | 1678 | O | GLU | A | 217 | 13064 | 7832 | 9886 | −1460 | 3109 | −623 | O |
| ATOM | 1679 | N | ARG | A | 218 | 63.099 | 69.398 | 33.276 | 1.00 | 77.05 | | N |
| ANISOU | 1679 | N | ARG | A | 218 | 13080 | 6944 | 9253 | −1067 | 3390 | −537 | N |
| ATOM | 1680 | CA | ARG | A | 218 | 63.714 | 70.013 | 34.477 | 1.00 | 75.94 | | C |
| ANISOU | 1680 | CA | ARG | A | 218 | 13157 | 6654 | 9043 | −1354 | 3457 | −711 | C |
| ATOM | 1681 | CB | ARG | A | 218 | 62.864 | 71.184 | 35.018 | 1.00 | 78.01 | | C |
| ANISOU | 1681 | CB | ARG | A | 218 | 13854 | 6599 | 9187 | −1164 | 3679 | −697 | C |
| ATOM | 1682 | CG | ARG | A | 218 | 62.929 | 72.474 | 34.213 | 1.00 | 93.55 | | C |
| ANISOU | 1682 | CG | ARG | A | 218 | 16264 | 8212 | 11069 | −1136 | 3932 | −646 | C |
| ATOM | 1683 | CD | ARG | A | 218 | 61.849 | 73.448 | 34.656 | 1.00 | 108.16 | | C |
| ANISOU | 1683 | CD | ARG | A | 218 | 18510 | 9791 | 12796 | −824 | 4130 | −593 | C |
| ATOM | 1684 | NE | ARG | A | 218 | 61.606 | 74.493 | 33.657 | 1.00 | 119.75 | | N |
| ANISOU | 1684 | NE | ARG | A | 218 | 20375 | 10952 | 14173 | −644 | 4358 | −483 | N |
| ATOM | 1685 | CZ | ARG | A | 218 | 60.627 | 75.389 | 33.731 | 1.00 | 133.99 | | C |
| ANISOU | 1685 | CZ | ARG | A | 218 | 22553 | 12510 | 15849 | −292 | 4549 | −401 | C |
| ATOM | 1686 | NH1 | ARG | A | 218 | 60.481 | 76.301 | 32.778 | 1.00 | 118.51 | | N |
| ANISOU | 1686 | NH1 | ARG | A | 218 | 20968 | 10268 | 13791 | −122 | 4754 | −296 | N |
| ATOM | 1687 | NH2 | ARG | A | 218 | 59.784 | 75.383 | 34.761 | 1.00 | 121.70 | | N |
| ANISOU | 1687 | NH2 | ARG | A | 218 | 21007 | 10986 | 14249 | −93 | 4543 | −422 | N |
| ATOM | 1688 | C | ARG | A | 218 | 63.811 | 68.943 | 35.565 | 1.00 | 75.46 | | C |
| ANISOU | 1688 | C | ARG | A | 218 | 12786 | 6866 | 9021 | −1434 | 3247 | −800 | C |
| ATOM | 1689 | O | ARG | A | 218 | 64.910 | 68.673 | 36.054 | 1.00 | 74.09 | | O |
| ANISOU | 1689 | O | ARG | A | 218 | 12503 | 6797 | 8850 | −1760 | 3151 | −947 | O |
| ATOM | 1690 | N | LEU | A | 219 | 62.646 | 68.312 | 35.896 | 1.00 | 69.49 | | N |
| ANISOU | 1690 | N | LEU | A | 219 | 11882 | 6240 | 8282 | −1125 | 3179 | −710 | N |
| ATOM | 1691 | CA | LEU | A | 219 | 62.468 | 67.246 | 36.884 | 1.00 | 68.14 | | C |
| ANISOU | 1691 | CA | LEU | A | 219 | 11447 | 6307 | 8134 | −1131 | 3003 | −760 | C |
| ATOM | 1692 | CB | LEU | A | 219 | 60.990 | 66.839 | 36.990 | 1.00 | 67.78 | | C |
| ANISOU | 1692 | CB | LEU | A | 219 | 11311 | 6349 | 8094 | −766 | 3005 | −639 | C |
| ATOM | 1693 | CG | LEU | A | 219 | 60.049 | 67.800 | 37.691 | 1.00 | 72.01 | | C |
| ANISOU | 1693 | CG | LEU | A | 219 | 12151 | 6677 | 8532 | −570 | 3186 | −634 | C |
| ATOM | 1694 | CD1 | LEU | A | 219 | 58.623 | 67.587 | 37.231 | 1.00 | 72.24 | | C |
| ANISOU | 1694 | CD1 | LEU | A | 219 | 12083 | 6801 | 8565 | −173 | 3222 | −485 | C |
| ATOM | 1695 | CD2 | LEU | A | 219 | 60.138 | 67.665 | 39.203 | 1.00 | 73.30 | | C |
| ANISOU | 1695 | CD2 | LEU | A | 219 | 12351 | 6858 | 8643 | −693 | 3151 | −760 | C |
| ATOM | 1696 | C | LEU | A | 219 | 63.299 | 66.016 | 36.561 | 1.00 | 71.05 | | C |
| ANISOU | 1696 | C | LEU | A | 219 | 11447 | 6962 | 8586 | −1287 | 2784 | −782 | C |
| ATOM | 1697 | O | LEU | A | 219 | 64.067 | 65.571 | 37.415 | 1.00 | 70.65 | | O |
| ANISOU | 1697 | O | LEU | A | 219 | 11294 | 7033 | 8518 | −1497 | 2665 | −907 | O |
| ATOM | 1698 | N | ILE | A | 220 | 63.151 | 65.461 | 35.341 | 1.00 | 66.94 | | N |
| ANISOU | 1698 | N | ILE | A | 220 | 10736 | 6557 | 8141 | −1164 | 2727 | −660 | N |
| ATOM | 1699 | CA | ILE | A | 220 | 63.887 | 64.266 | 34.930 | 1.00 | 67.05 | | C |
| ANISOU | 1699 | CA | ILE | A | 220 | 10414 | 6835 | 8226 | −1272 | 2526 | −666 | C |
| ATOM | 1700 | CB | ILE | A | 220 | 63.452 | 63.711 | 33.542 | 1.00 | 70.37 | | C |
| ANISOU | 1700 | CB | ILE | A | 220 | 10658 | 7359 | 8720 | −1073 | 2486 | −513 | C |
| ATOM | 1701 | CG1 | ILE | A | 220 | 61.933 | 63.373 | 33.476 | 1.00 | 71.05 | | C |
| ANISOU | 1701 | CG1 | ILE | A | 220 | 10683 | 7500 | 8812 | −741 | 2500 | −394 | C |
| ATOM | 1702 | CD1 | ILE | A | 220 | 61.452 | 62.000 | 34.131 | 1.00 | 80.23 | | C |
| ANISOU | 1702 | CD1 | ILE | A | 220 | 11569 | 8912 | 10004 | −686 | 2332 | −393 | C |
| ATOM | 1703 | CG2 | ILE | A | 220 | 64.293 | 62.509 | 33.120 | 1.00 | 70.97 | | C |
| ANISOU | 1703 | CG2 | ILE | A | 220 | 10424 | 7685 | 8857 | −1190 | 2291 | −526 | C |
| ATOM | 1704 | C | ILE | A | 220 | 65.382 | 64.508 | 35.043 | 1.00 | 71.88 | | C |
| ANISOU | 1704 | C | ILE | A | 220 | 11032 | 7459 | 8820 | −1619 | 2495 | −809 | C |
| ATOM | 1705 | O | ILE | A | 220 | 66.095 | 63.619 | 35.505 | 1.00 | 71.65 | | O |
| ANISOU | 1705 | O | ILE | A | 220 | 10779 | 7649 | 8796 | −1751 | 2323 | −889 | O |
| ATOM | 1706 | N | GLN | A | 221 | 65.843 | 65.727 | 34.712 | 1.00 | 69.32 | | N |
| ANISOU | 1706 | N | GLN | A | 221 | 10979 | 6904 | 8456 | −1767 | 2669 | −851 | N |
| ATOM | 1707 | CA | GLN | A | 221 | 67.259 | 66.060 | 34.842 | 1.00 | 69.89 | | C |
| ANISOU | 1707 | CA | GLN | A | 221 | 11060 | 6998 | 8499 | −2136 | 2664 | −1009 | C |
| ATOM | 1708 | C | GLN | A | 221 | 67.713 | 66.183 | 36.301 | 1.00 | 74.73 | | C |
| ANISOU | 1708 | C | GLN | A | 221 | 11728 | 7633 | 9032 | −2339 | 2625 | −1188 | C |
| ATOM | 1709 | O | GLN | A | 221 | 68.873 | 65.875 | 36.577 | 1.00 | 75.25 | | O |
| ANISOU | 1709 | O | GLN | A | 221 | 11640 | 7875 | 9076 | −2609 | 2521 | −1330 | O |
| ATOM | 1710 | CB | GLN | A | 221 | 67.654 | 67.300 | 34.021 | 1.00 | 71.31 | | C |
| ANISOU | 1710 | CB | GLN | A | 221 | 11523 | 6919 | 8650 | −2274 | 2879 | −1009 | C |
| ATOM | 1711 | CG | GLN | A | 221 | 69.021 | 67.169 | 33.323 | 1.00 | 77.90 | | C |
| ANISOU | 1711 | CG | GLN | A | 221 | 12197 | 7895 | 9507 | −2574 | 2834 | −1089 | C |
| ATOM | 1712 | CD | GLN | A | 221 | 69.077 | 66.070 | 32.269 | 1.00 | 86.50 | | C |
| ANISOU | 1712 | CD | GLN | A | 221 | 12957 | 9222 | 10687 | −2422 | 2684 | −967 | C |
| ATOM | 1713 | OE1 | GLN | A | 221 | 68.064 | 65.669 | 31.671 | 1.00 | 78.10 | | O |
| ANISOU | 1713 | OE1 | GLN | A | 221 | 11857 | 8146 | 9670 | −2103 | 2673 | −799 | O |
| ATOM | 1714 | NE2 | GLN | A | 221 | 70.275 | 65.556 | 32.016 | 1.00 | 74.04 | | N |
| ANISOU | 1714 | NE2 | GLN | A | 221 | 11125 | 7881 | 9124 | −2646 | 2565 | −1059 | N |
| ATOM | 1715 | N | LYS | A | 222 | 66.818 | 66.599 | 37.234 | 1.00 | 70.70 | | N |
| ANISOU | 1715 | N | LYS | A | 222 | 11421 | 6973 | 8470 | −2200 | 2702 | −1187 | N |
| ATOM | 1716 | CA | LYS | A | 222 | 67.165 | 66.685 | 38.667 | 1.00 | 70.37 | | C |
| ANISOU | 1716 | CA | LYS | A | 222 | 11439 | 6956 | 8342 | −2368 | 2659 | −1354 | C |
| ATOM | 1717 | CB | LYS | A | 222 | 66.035 | 67.329 | 39.516 | 1.00 | 72.91 | | C |
| ANISOU | 1717 | CB | LYS | A | 222 | 12046 | 7054 | 8601 | −2181 | 2796 | −1329 | C |
| ATOM | 1718 | CG | LYS | A | 222 | 65.772 | 68.810 | 39.267 | 1.00 | 86.93 | | C |
| ANISOU | 1718 | CG | LYS | A | 222 | 14252 | 8465 | 10314 | −2211 | 3057 | −1337 | C |
| ATOM | 1719 | CD | LYS | A | 222 | 64.434 | 69.282 | 39.858 | 1.00 | 94.08 | | C |
| ANISOU | 1719 | CD | LYS | A | 222 | 15399 | 9181 | 11165 | −1911 | 3187 | −1263 | C |
| ATOM | 1720 | CE | LYS | A | 222 | 64.014 | 70.629 | 39.301 | 1.00 | 100.94 | | C |
| ANISOU | 1720 | CE | LYS | A | 222 | 16680 | 9695 | 11978 | −1811 | 3446 | −1202 | C |
| ATOM | 1721 | NZ | LYS | A | 222 | 62.792 | 71.159 | 39.961 | 1.00 | 107.25 | | N |
| ANISOU | 1721 | NZ | LYS | A | 222 | 17725 | 10324 | 12703 | −1512 | 3578 | −1147 | N |
| ATOM | 1722 | C | LYS | A | 222 | 67.433 | 65.260 | 39.170 | 1.00 | 72.99 | | C |
| ANISOU | 1722 | C | LYS | A | 222 | 11414 | 7624 | 8694 | −2338 | 2408 | −1375 | C |
| ATOM | 1723 | O | LYS | A | 222 | 68.413 | 65.030 | 39.886 | 1.00 | 73.88 | | O |
| ANISOU | 1723 | O | LYS | A | 222 | 11428 | 7896 | 8748 | −2565 | 2296 | −1534 | O |
| ATOM | 1724 | N | ALA | A | 223 | 66.566 | 64.308 | 38.758 | 1.00 | 66.37 | | N |
| ANISOU | 1724 | N | ALA | A | 223 | 10392 | 6898 | 7926 | −2057 | 2324 | −1218 | N |
| ATOM | 1725 | CA | ALA | A | 223 | 66.656 | 62.902 | 39.105 | 1.00 | 65.04 | | C |
| ANISOU | 1725 | CA | ALA | A | 223 | 9933 | 7006 | 7772 | −1989 | 2112 | −1208 | C |
| ATOM | 1726 | CB | ALA | A | 223 | 65.440 | 62.169 | 38.576 | 1.00 | 65.76 | | C |
| ANISOU | 1726 | CB | ALA | A | 223 | 9920 | 7135 | 7933 | −1687 | 2097 | −1032 | C |
| ATOM | 1727 | C | ALA | A | 223 | 67.931 | 62.307 | 38.521 | 1.00 | 67.60 | | C |
| ANISOU | 1727 | C | ALA | A | 223 | 10013 | 7555 | 8116 | −2155 | 1966 | −1263 | C |
| ATOM | 1728 | O | ALA | A | 223 | 68.705 | 61.709 | 39.265 | 1.00 | 66.31 | | O |
| ANISOU | 1728 | O | ALA | A | 223 | 9710 | 7593 | 7892 | −2258 | 1814 | −1373 | O |
| ATOM | 1729 | N | ILE | A | 224 | 68.169 | 62.517 | 37.200 | 1.00 | 64.82 | | N |
| ANISOU | 1729 | N | ILE | A | 224 | 9618 | 7176 | 7836 | −2169 | 2018 | −1189 | N |
| ATOM | 1730 | CA | ILE | A | 224 | 69.347 | 62.047 | 36.449 | 1.00 | 64.75 | | C |
| ANISOU | 1730 | CA | ILE | A | 224 | 9379 | 7372 | 7849 | −2314 | 1908 | −1228 | C |
| ATOM | 1731 | CB | ILE | A | 224 | 69.259 | 62.438 | 34.937 | 1.00 | 67.11 | | C |
| ANISOU | 1731 | CB | ILE | A | 224 | 9700 | 7571 | 8227 | −2275 | 2015 | −1111 | C |
| ATOM | 1732 | CG1 | ILE | A | 224 | 68.179 | 61.623 | 34.211 | 1.00 | 66.44 | | C |
| ANISOU | 1732 | CG1 | ILE | A | 224 | 9502 | 7521 | 8219 | −1963 | 1968 | −923 | C |
| ATOM | 1733 | CD1 | ILE | A | 224 | 67.782 | 62.181 | 32.916 | 1.00 | 65.62 | | C |
| ANISOU | 1733 | CD1 | ILE | A | 224 | 9495 | 7268 | 8168 | −1862 | 2101 | −799 | C |
| ATOM | 1734 | CG2 | ILE | A | 224 | 70.623 | 62.304 | 34.211 | 1.00 | 68.21 | | C |
| ANISOU | 1734 | CG2 | ILE | A | 224 | 9662 | 7883 | 8371 | −2499 | 1959 | −1188 | C |
| ATOM | 1735 | C | ILE | A | 224 | 70.664 | 62.501 | 37.124 | 1.00 | 71.12 | | C |
| ANISOU | 1735 | C | ILE | A | 224 | 10178 | 8275 | 8568 | −2638 | 1879 | −1440 | C |
| ATOM | 1736 | O | ILE | A | 224 | 71.565 | 61.674 | 37.293 | 1.00 | 71.04 | | O |
| ANISOU | 1736 | O | ILE | A | 224 | 9921 | 8547 | 8525 | −2705 | 1703 | −1518 | O |
| ATOM | 1737 | N | HIS | A | 225 | 70.754 | 63.801 | 37.526 | 1.00 | 68.51 | | N |
| ANISOU | 1737 | N | HIS | A | 225 | 10127 | 7717 | 8186 | −2830 | 2055 | −1538 | N |
| ATOM | 1738 | CA | HIS | A | 225 | 71.920 | 64.400 | 38.201 | 1.00 | 68.03 | | C |
| ANISOU | 1738 | CA | HIS | A | 225 | 10098 | 7723 | 8029 | −3179 | 2061 | −1762 | C |
| ATOM | 1739 | CB | HIS | A | 225 | 71.703 | 65.909 | 38.461 | 1.00 | 68.69 | | C |
| ANISOU | 1739 | CB | HIS | A | 225 | 10573 | 7462 | 8065 | −3347 | 2306 | −1828 | C |
| ATOM | 1740 | C | HIS | A | 225 | 72.173 | 63.684 | 39.516 | 1.00 | 70.10 | | C |
| ANISOU | 1740 | C | HIS | A | 225 | 10233 | 8200 | 8201 | −3177 | 1882 | −1878 | C |
| ATOM | 1741 | O | HIS | A | 225 | 73.321 | 63.376 | 39.845 | 1.00 | 69.26 | | O |
| ANISOU | 1741 | O | HIS | A | 225 | 9934 | 8358 | 8022 | −3376 | 1756 | −2039 | O |
| ATOM | 1742 | N | LEU | A | 226 | 71.078 | 63.390 | 40.237 | 1.00 | 65.77 | | N |
| ANISOU | 1742 | N | LEU | A | 226 | 9788 | 7555 | 7647 | −2938 | 1873 | −1796 | N |
| ATOM | 1743 | CA | LEU | A | 226 | 71.081 | 62.732 | 41.536 | 1.00 | 64.96 | | C |
| ANISOU | 1743 | CA | LEU | A | 226 | 9626 | 7607 | 7451 | −2887 | 1728 | −1877 | C |
| ATOM | 1744 | CB | LEU | A | 226 | 69.709 | 62.830 | 42.204 | 1.00 | 64.55 | | C |
| ANISOU | 1744 | CB | LEU | A | 226 | 9780 | 7349 | 7397 | −2662 | 1812 | −1781 | C |
| ATOM | 1745 | CG | LEU | A | 226 | 69.535 | 63.978 | 43.151 | 1.00 | 68.61 | | C |
| ANISOU | 1745 | CG | LEU | A | 226 | 10600 | 7642 | 7825 | −2793 | 1958 | −1898 | C |
| ATOM | 1746 | CD1 | LEU | A | 226 | 68.072 | 64.174 | 43.484 | 1.00 | 68.64 | | C |
| ANISOU | 1746 | CD1 | LEU | A | 226 | 10804 | 7426 | 7849 | −2533 | 2080 | −1769 | C |
| ATOM | 1747 | CD2 | LEU | A | 226 | 70.359 | 63.764 | 44.411 | 1.00 | 70.47 | | C |
| ANISOU | 1747 | CD2 | LEU | A | 226 | 10785 | 8066 | 7923 | −2962 | 1825 | −2097 | C |
| ATOM | 1748 | C | LEU | A | 226 | 71.515 | 61.297 | 41.453 | 1.00 | 68.49 | | C |
| ANISOU | 1748 | C | LEU | A | 226 | 9758 | 8372 | 7892 | −2763 | 1502 | −1845 | C |
| ATOM | 1749 | O | LEU | A | 226 | 72.316 | 60.872 | 42.277 | 1.00 | 68.68 | | O |
| ANISOU | 1749 | O | LEU | A | 226 | 9659 | 8629 | 7807 | −2848 | 1354 | −1985 | O |
| ATOM | 1750 | N | LEU | A | 227 | 70.994 | 60.545 | 40.479 | 1.00 | 63.94 | | N |
| ANISOU | 1750 | N | LEU | A | 227 | 9064 | 7812 | 7420 | −2551 | 1474 | −1667 | N |
| ATOM | 1751 | CA | LEU | A | 227 | 71.373 | 59.151 | 40.336 | 1.00 | 63.33 | | C |
| ANISOU | 1751 | CA | LEU | A | 227 | 8726 | 8005 | 7331 | −2416 | 1274 | −1626 | C |
| ATOM | 1752 | CB | LEU | A | 227 | 70.386 | 58.407 | 39.448 | 1.00 | 62.29 | | C |
| ANISOU | 1752 | CB | LEU | A | 227 | 8548 | 7812 | 7308 | −2165 | 1282 | −1420 | C |
| ATOM | 1753 | CG | LEU | A | 227 | 69.554 | 57.342 | 40.108 | 1.00 | 65.13 | | C |
| ANISOU | 1753 | CG | LEU | A | 227 | 8906 | 8205 | 7635 | −1949 | 1202 | −1343 | C |
| ATOM | 1754 | CD1 | LEU | A | 227 | 68.638 | 56.707 | 39.096 | 1.00 | 65.26 | | C |
| ANISOU | 1754 | CD1 | LEU | A | 227 | 8859 | 8180 | 7757 | −1751 | 1220 | −1162 | C |
| ATOM | 1755 | CD2 | LEU | A | 227 | 70.421 | 56.275 | 40.729 | 1.00 | 64.94 | | C |
| ANISOU | 1755 | CD2 | LEU | A | 227 | 8734 | 8442 | 7497 | −1932 | 1003 | −1424 | C |
| ATOM | 1756 | C | LEU | A | 227 | 72.798 | 59.026 | 39.797 | 1.00 | 70.62 | | C |
| ANISOU | 1756 | C | LEU | A | 227 | 9425 | 9179 | 8227 | −2589 | 1175 | −1732 | C |
| ATOM | 1757 | O | LEU | A | 227 | 73.567 | 58.221 | 40.329 | 1.00 | 71.41 | | O |
| ANISOU | 1757 | O | LEU | A | 227 | 9347 | 9552 | 8232 | −2565 | 995 | −1811 | O |
| ATOM | 1758 | N | GLN | A | 228 | 73.166 | 59.859 | 38.784 | 1.00 | 67.72 | | N |
| ANISOU | 1758 | N | GLN | A | 228 | 9078 | 8725 | 7928 | −2760 | 1301 | −1739 | N |
| ATOM | 1759 | CA | GLN | A | 228 | 74.506 | 59.873 | 38.186 | 1.00 | 67.68 | | C |
| ANISOU | 1759 | CA | GLN | A | 228 | 8859 | 8955 | 7900 | −2959 | 1242 | −1847 | C |
| ATOM | 1760 | CB | GLN | A | 228 | 74.616 | 60.949 | 37.085 | 1.00 | 68.96 | | C |
| ANISOU | 1760 | CB | GLN | A | 228 | 9131 | 8925 | 8144 | −3135 | 1440 | −1822 | C |
| ATOM | 1761 | CG | GLN | A | 228 | 75.812 | 60.778 | 36.155 | 1.00 | 94.45 | | C |
| ANISOU | 1761 | CG | GLN | A | 228 | 12117 | 12391 | 11379 | −3289 | 1396 | −1878 | C |
| ATOM | 1762 | CD | GLN | A | 228 | 75.445 | 60.221 | 34.793 | 1.00 | 131.19 | | C |
| ANISOU | 1762 | CD | GLN | A | 228 | 16681 | 17021 | 16143 | −3091 | 1400 | −1686 | C |
| ATOM | 1763 | OE1 | GLN | A | 228 | 74.446 | 60.614 | 34.169 | 1.00 | 130.79 | | O |
| ANISOU | 1763 | OE1 | GLN | A | 228 | 16816 | 16696 | 16181 | −2960 | 1533 | −1530 | O |
| ATOM | 1764 | NE2 | GLN | A | 228 | 76.265 | 59.306 | 34.281 | 1.00 | 126.51 | | N |
| ANISOU | 1764 | NE2 | GLN | A | 228 | 15799 | 16732 | 15537 | −3055 | 1252 | −1699 | N |
| ATOM | 1765 | C | GLN | A | 228 | 75.555 | 60.092 | 39.290 | 1.00 | 71.94 | | C |
| ANISOU | 1765 | C | GLN | A | 228 | 9329 | 9711 | 8292 | −3184 | 1154 | −2084 | C |
| ATOM | 1766 | O | GLN | A | 228 | 76.501 | 59.308 | 39.389 | 1.00 | 71.82 | | O |
| ANISOU | 1766 | O | GLN | A | 228 | 9050 | 10038 | 8200 | −3184 | 978 | −2169 | O |
| ATOM | 1767 | N | GLU | A | 229 | 75.327 | 61.088 | 40.171 | 1.00 | 68.03 | | N |
| ANISOU | 1767 | N | GLU | A | 229 | 9073 | 9031 | 7743 | −3342 | 1267 | −2190 | N |
| ATOM | 1768 | CA | GLU | A | 229 | 76.246 | 61.401 | 41.262 | 1.00 | 67.84 | | C |
| ANISOU | 1768 | CA | GLU | A | 229 | 9007 | 9200 | 7569 | −3573 | 1194 | −2429 | C |
| ATOM | 1769 | CB | GLU | A | 229 | 75.972 | 62.798 | 41.843 | 1.00 | 69.24 | | C |
| ANISOU | 1769 | CB | GLU | A | 229 | 9507 | 9084 | 7715 | −3812 | 1392 | −2535 | C |
| ATOM | 1770 | C | GLU | A | 229 | 76.277 | 60.336 | 42.365 | 1.00 | 71.62 | | C |
| ANISOU | 1770 | C | GLU | A | 229 | 9374 | 9909 | 7931 | −3371 | 981 | −2461 | C |
| ATOM | 1771 | O | GLU | A | 229 | 77.350 | 60.084 | 42.924 | 1.00 | 71.06 | | O |
| ANISOU | 1771 | O | GLU | A | 229 | 9109 | 10166 | 7725 | −3483 | 833 | −2640 | O |
| ATOM | 1772 | N | THR | A | 230 | 75.110 | 59.725 | 42.691 | 1.00 | 68.13 | | N |
| ANISOU | 1772 | N | THR | A | 230 | 9054 | 9307 | 7525 | −3076 | 971 | −2294 | N |
| ATOM | 1773 | CA | THR | A | 230 | 75.009 | 58.702 | 43.747 | 1.00 | 67.54 | | C |
| ANISOU | 1773 | CA | THR | A | 230 | 8933 | 9396 | 7333 | −2866 | 798 | −2299 | C |
| ATOM | 1774 | CB | THR | A | 230 | 73.543 | 58.461 | 44.178 | 1.00 | 74.77 | | C |
| ANISOU | 1774 | CB | THR | A | 230 | 10071 | 10039 | 8298 | −2631 | 876 | −2134 | C |
| ATOM | 1775 | OG1 | THR | A | 230 | 72.972 | 59.699 | 44.614 | 1.00 | 74.08 | | O |
| ANISOU | 1775 | OG1 | THR | A | 230 | 10251 | 9669 | 8227 | −2767 | 1062 | −2177 | O |
| ATOM | 1776 | CG2 | THR | A | 230 | 73.419 | 57.448 | 45.314 | 1.00 | 73.84 | | C |
| ANISOU | 1776 | CG2 | THR | A | 230 | 9952 | 10056 | 8047 | −2432 | 726 | −2138 | C |
| ATOM | 1777 | C | THR | A | 230 | 75.756 | 57.448 | 43.321 | 1.00 | 70.31 | | C |
| ANISOU | 1777 | C | THR | A | 230 | 8995 | 10077 | 7643 | −2712 | 598 | −2273 | C |
| ATOM | 1778 | O | THR | A | 230 | 76.455 | 56.844 | 44.148 | 1.00 | 69.90 | | O |
| ANISOU | 1778 | O | THR | A | 230 | 8824 | 10305 | 7432 | −2660 | 425 | −2386 | O |
| ATOM | 1779 | N | LEU | A | 231 | 75.637 | 57.091 | 42.018 | 1.00 | 65.46 | | N |
| ANISOU | 1779 | N | LEU | A | 231 | 8279 | 9436 | 7159 | −2632 | 626 | −2130 | N |
| ATOM | 1780 | CA | LEU | A | 231 | 76.289 | 55.923 | 41.435 | 1.00 | 64.56 | | C |
| ANISOU | 1780 | CA | LEU | A | 231 | 7913 | 9598 | 7017 | −2470 | 461 | −2086 | C |
| ATOM | 1781 | CB | LEU | A | 231 | 75.641 | 55.498 | 40.111 | 1.00 | 63.72 | | C |
| ANISOU | 1781 | CB | LEU | A | 231 | 7792 | 9349 | 7070 | −2326 | 523 | −1880 | C |
| ATOM | 1782 | CG | LEU | A | 231 | 74.206 | 54.987 | 40.210 | 1.00 | 67.28 | | C |
| ANISOU | 1782 | CG | LEU | A | 231 | 8421 | 9553 | 7589 | −2096 | 575 | −1696 | C |
| ATOM | 1783 | CD1 | LEU | A | 231 | 73.488 | 55.161 | 38.920 | 1.00 | 67.28 | | C |
| ANISOU | 1783 | CD1 | LEU | A | 231 | 8442 | 9366 | 7754 | −2052 | 695 | −1535 | C |
| ATOM | 1784 | CD2 | LEU | A | 231 | 74.150 | 53.544 | 40.633 | 1.00 | 69.14 | | C |
| ANISOU | 1784 | CD2 | LEU | A | 231 | 8606 | 9936 | 7729 | −1842 | 412 | −1638 | C |
| ATOM | 1785 | C | LEU | A | 231 | 77.783 | 56.136 | 41.290 | 1.00 | 70.40 | | C |
| ANISOU | 1785 | C | LEU | A | 231 | 8399 | 10684 | 7664 | −2665 | 368 | −2275 | C |
| ATOM | 1786 | O | LEU | A | 231 | 78.547 | 55.289 | 41.758 | 1.00 | 71.18 | | O |
| ANISOU | 1786 | O | LEU | A | 231 | 8322 | 11104 | 7618 | −2538 | 178 | −2347 | O |
| ATOM | 1787 | N | ALA | A | 232 | 78.203 | 57.290 | 40.719 | 1.00 | 66.60 | | N |
| ANISOU | 1787 | N | ALA | A | 232 | 7910 | 10149 | 7246 | −2973 | 507 | −2366 | N |
| ATOM | 1788 | CA | ALA | A | 232 | 79.616 | 57.647 | 40.548 | 1.00 | 66.52 | | C |
| ANISOU | 1788 | CA | ALA | A | 232 | 7651 | 10474 | 7151 | −3224 | 452 | −2569 | C |
| ATOM | 1789 | CB | ALA | A | 232 | 79.738 | 58.962 | 39.800 | 1.00 | 67.01 | | C |
| ANISOU | 1789 | CB | ALA | A | 232 | 7799 | 10347 | 7313 | −3561 | 669 | −2616 | C |
| ATOM | 1790 | C | ALA | A | 232 | 80.410 | 57.706 | 41.881 | 1.00 | 72.66 | | C |
| ANISOU | 1790 | C | ALA | A | 232 | 8343 | 11536 | 7727 | −3329 | 318 | −2802 | C |
| ATOM | 1791 | O | ALA | A | 232 | 81.612 | 57.423 | 41.884 | 1.00 | 72.84 | | O |
| ANISOU | 1791 | O | ALA | A | 232 | 8075 | 11967 | 7632 | −3404 | 185 | −2958 | O |
| ATOM | 1792 | N | ARG | A | 233 | 79.743 | 58.054 | 43.002 | 1.00 | 69.99 | | N |
| ANISOU | 1792 | N | ARG | A | 233 | 8247 | 11006 | 7340 | −3321 | 350 | −2828 | N |
| ATOM | 1793 | CA | ARG | A | 233 | 80.382 | 58.124 | 44.316 | 1.00 | 70.38 | | C |
| ANISOU | 1793 | CA | ARG | A | 233 | 8253 | 11300 | 7190 | −3403 | 225 | −3043 | C |
| ATOM | 1794 | CB | ARG | A | 233 | 79.700 | 59.180 | 45.206 | 1.00 | 71.28 | | C |
| ANISOU | 1794 | CB | ARG | A | 233 | 8687 | 11094 | 7301 | −3573 | 375 | −3104 | C |
| ATOM | 1795 | C | ARG | A | 233 | 80.459 | 56.755 | 45.025 | 1.00 | 75.93 | | C |
| ANISOU | 1795 | C | ARG | A | 233 | 8861 | 12243 | 7745 | −3039 | −3 | −3001 | C |
| ATOM | 1796 | O | ARG | A | 233 | 81.082 | 56.666 | 46.086 | 1.00 | 76.11 | | O |
| ANISOU | 1796 | O | ARG | A | 233 | 8814 | 12530 | 7575 | −3054 | −138 | −3177 | O |
| ATOM | 1797 | N | ASP | A | 234 | 79.839 | 55.690 | 44.454 | 1.00 | 73.08 | | N |
| ANISOU | 1797 | N | ASP | A | 234 | 8516 | 11792 | 7459 | −2713 | −41 | −2776 | N |
| ATOM | 1798 | CA | ASP | A | 234 | 79.878 | 54.354 | 45.067 | 1.00 | 72.87 | | C |
| ANISOU | 1798 | CA | ASP | A | 234 | 8454 | 11951 | 7284 | −2359 | −233 | −2721 | C |
| ATOM | 1799 | CB | ASP | A | 234 | 78.556 | 53.580 | 44.904 | 1.00 | 74.27 | | C |
| ANISOU | 1799 | CB | ASP | A | 234 | 8854 | 11803 | 7563 | −2081 | −176 | −2467 | C |
| ATOM | 1800 | CG | ASP | A | 234 | 78.423 | 52.423 | 45.881 | 1.00 | 77.32 | | C |
| ANISOU | 1800 | CG | ASP | A | 234 | 9326 | 12281 | 7771 | −1769 | −324 | −2428 | C |
| ATOM | 1801 | OD2 | ASP | A | 234 | 77.812 | 52.618 | 46.953 | 1.00 | 76.81 | | O |
| ANISOU | 1801 | OD2 | ASP | A | 234 | 9474 | 12069 | 7642 | −1761 | −290 | −2449 | O |
| ATOM | 1802 | OD1 | ASP | A | 234 | 78.939 | 51.325 | 45.576 | 1.00 | 77.04 | | O |
| ANISOU | 1802 | OD1 | ASP | A | 234 | 9165 | 12459 | 7648 | −1526 | −465 | −2377 | O |
| ATOM | 1803 | C | ASP | A | 234 | 81.084 | 53.555 | 44.575 | 1.00 | 76.41 | | C |
| ANISOU | 1803 | C | ASP | A | 234 | 8575 | 12834 | 7624 | −2246 | −412 | −2783 | C |
| ATOM | 1804 | O | ASP | A | 234 | 81.117 | 53.114 | 43.424 | 1.00 | 74.18 | | O |
| ANISOU | 1804 | O | ASP | A | 234 | 8186 | 12549 | 7452 | −2168 | −394 | −2662 | O |
| ATOM | 1805 | N | GLU | A | 235 | 82.075 | 53.384 | 45.474 | 1.00 | 74.38 | | N |
| ANISOU | 1805 | N | GLU | A | 235 | 8154 | 12968 | 7138 | −2230 | −585 | −2981 | N |
| ATOM | 1806 | CA | GLU | A | 235 | 83.352 | 52.712 | 45.215 | 1.00 | 74.33 | | C |
| ANISOU | 1806 | CA | GLU | A | 235 | 7808 | 13455 | 6981 | −2114 | −772 | −3085 | C |
| ATOM | 1807 | CB | GLU | A | 235 | 84.308 | 52.864 | 46.412 | 1.00 | 76.05 | | C |
| ANISOU | 1807 | CB | GLU | A | 235 | 7873 | 14087 | 6937 | −2159 | −939 | −3343 | C |
| ATOM | 1808 | CG | GLU | A | 235 | 85.061 | 54.182 | 46.431 | 1.00 | 91.13 | | C |
| ANISOU | 1808 | CG | GLU | A | 235 | 9617 | 16160 | 8850 | −2629 | −868 | −3595 | C |
| ATOM | 1809 | CD | GLU | A | 235 | 86.308 | 54.183 | 47.294 | 1.00 | 127.51 | | C |
| ANISOU | 1809 | CD | GLU | A | 235 | 13943 | 21321 | 13184 | −2673 | −1065 | −3876 | C |
| ATOM | 1810 | OE1 | GLU | A | 235 | 87.258 | 53.435 | 46.964 | 1.00 | 132.13 | | O |
| ANISOU | 1810 | OE1 | GLU | A | 235 | 14213 | 22353 | 13638 | −2487 | −1231 | −3928 | O |
| ATOM | 1811 | OE2 | GLU | A | 235 | 86.342 | 54.939 | 48.293 | 1.00 | 127.18 | | O |
| ANISOU | 1811 | OE2 | GLU | A | 235 | 13990 | 21284 | 13048 | −2887 | −1056 | −4050 | O |
| ATOM | 1812 | C | GLU | A | 235 | 83.242 | 51.237 | 44.817 | 1.00 | 75.56 | | C |
| ANISOU | 1812 | C | GLU | A | 235 | 7955 | 13664 | 7091 | −1682 | −889 | −2898 | C |
| ATOM | 1813 | O | GLU | A | 235 | 83.931 | 50.821 | 43.882 | 1.00 | 75.68 | | O |
| ANISOU | 1813 | O | GLU | A | 235 | 7736 | 13897 | 7120 | −1618 | −938 | −2883 | O |
| ATOM | 1814 | N | LEU | A | 236 | 82.420 | 50.448 | 45.541 | 1.00 | 69.01 | | N |
| ANISOU | 1814 | N | LEU | A | 236 | 7389 | 12640 | 6193 | −1394 | −925 | −2766 | N |
| ATOM | 1815 | CA | LEU | A | 236 | 82.261 | 49.017 | 45.292 | 1.00 | 67.13 | | C |
| ANISOU | 1815 | CA | LEU | A | 236 | 7212 | 12410 | 5885 | −989 | −1020 | −2593 | C |
| ATOM | 1816 | CB | LEU | A | 236 | 81.435 | 48.358 | 46.413 | 1.00 | 66.25 | | C |
| ANISOU | 1816 | CB | LEU | A | 236 | 7423 | 12092 | 5658 | −750 | −1041 | −2500 | C |
| ATOM | 1817 | CG | LEU | A | 236 | 81.578 | 46.849 | 46.687 | 1.00 | 69.17 | | C |
| ANISOU | 1817 | CG | LEU | A | 236 | 7876 | 12591 | 5816 | −309 | −1194 | −2409 | C |
| ATOM | 1818 | CD1 | LEU | A | 236 | 82.986 | 46.447 | 46.954 | 1.00 | 69.04 | | C |
| ANISOU | 1818 | CD1 | LEU | A | 236 | 7585 | 13101 | 5545 | −152 | −1407 | −2580 | C |
| ATOM | 1819 | CD2 | LEU | A | 236 | 80.970 | 46.013 | 45.615 | 1.00 | 70.58 | | C |
| ANISOU | 1819 | CD2 | LEU | A | 236 | 8142 | 12556 | 6119 | −141 | −1133 | −2193 | C |
| ATOM | 1820 | C | LEU | A | 236 | 81.627 | 48.786 | 43.953 | 1.00 | 72.21 | | C |
| ANISOU | 1820 | C | LEU | A | 236 | 7890 | 12791 | 6756 | −981 | −897 | −2397 | C |
| ATOM | 1821 | O | LEU | A | 236 | 82.061 | 47.885 | 43.237 | 1.00 | 72.35 | | O |
| ANISOU | 1821 | O | LEU | A | 236 | 7787 | 12969 | 6735 | −766 | −980 | −2330 | O |
| ATOM | 1822 | N | LEU | A | 237 | 80.619 | 49.609 | 43.596 | 1.00 | 69.69 | | N |
| ANISOU | 1822 | N | LEU | A | 237 | 7736 | 12083 | 6661 | −1203 | −700 | −2311 | N |
| ATOM | 1823 | CA | LEU | A | 237 | 79.912 | 49.512 | 42.314 | 1.00 | 69.77 | | C |
| ANISOU | 1823 | CA | LEU | A | 237 | 7787 | 11832 | 6892 | −1211 | −571 | −2128 | C |
| ATOM | 1824 | CB | LEU | A | 237 | 78.705 | 50.457 | 42.275 | 1.00 | 69.40 | | C |
| ANISOU | 1824 | CB | LEU | A | 237 | 7958 | 11372 | 7038 | −1412 | −366 | −2052 | C |
| ATOM | 1825 | CG | LEU | A | 237 | 77.876 | 50.387 | 41.015 | 1.00 | 72.87 | | C |
| ANISOU | 1825 | CG | LEU | A | 237 | 8452 | 11546 | 7689 | −1399 | −236 | −1864 | C |
| ATOM | 1826 | CD1 | LEU | A | 237 | 76.683 | 49.502 | 41.216 | 1.00 | 72.93 | | C |
| ANISOU | 1826 | CD1 | LEU | A | 237 | 8691 | 11301 | 7716 | −1179 | −206 | −1689 | C |
| ATOM | 1827 | CD2 | LEU | A | 237 | 77.472 | 51.766 | 40.566 | 1.00 | 74.08 | | C |
| ANISOU | 1827 | CD2 | LEU | A | 237 | 8647 | 11484 | 8017 | −1693 | −50 | −1881 | C |
| ATOM | 1828 | C | LEU | A | 237 | 80.847 | 49.804 | 41.150 | 1.00 | 75.19 | | C |
| ANISOU | 1828 | C | LEU | A | 237 | 8184 | 12744 | 7642 | −1341 | −576 | −2185 | C |
| ATOM | 1829 | O | LEU | A | 237 | 80.958 | 48.974 | 40.253 | 1.00 | 75.11 | | O |
| ANISOU | 1829 | O | LEU | A | 237 | 8100 | 12782 | 7657 | −1156 | −616 | −2073 | O |
| ATOM | 1830 | N | GLN | A | 238 | 81.543 | 50.960 | 41.192 | 1.00 | 72.59 | | N |
| ANISOU | 1830 | N | GLN | A | 238 | 7699 | 12558 | 7323 | −1666 | −531 | −2369 | N |
| ATOM | 1831 | CA | GLN | A | 238 | 82.487 | 51.401 | 40.170 | 1.00 | 72.92 | | C |
| ANISOU | 1831 | CA | GLN | A | 238 | 7465 | 12826 | 7415 | −1852 | −511 | −2452 | C |
| ATOM | 1832 | CB | GLN | A | 238 | 83.010 | 52.815 | 40.461 | 1.00 | 74.03 | | C |
| ANISOU | 1832 | CB | GLN | A | 238 | 7532 | 13026 | 7568 | −2273 | −415 | −2659 | C |
| ATOM | 1833 | CG | GLN | A | 238 | 81.981 | 53.940 | 40.278 | 1.00 | 82.18 | | C |
| ANISOU | 1833 | CG | GLN | A | 238 | 8835 | 13596 | 8792 | −2515 | −184 | −2589 | C |
| ATOM | 1834 | CD | GLN | A | 238 | 81.235 | 53.932 | 38.965 | 1.00 | 101.24 | | C |
| ANISOU | 1834 | CD | GLN | A | 238 | 11339 | 15714 | 11414 | −2474 | −43 | −2380 | C |
| ATOM | 1835 | OE1 | GLN | A | 238 | 80.006 | 54.000 | 38.947 | 1.00 | 96.78 | | O |
| ANISOU | 1835 | OE1 | GLN | A | 238 | 11031 | 14776 | 10965 | −2399 | 65 | −2224 | O |
| ATOM | 1836 | NE2 | GLN | A | 238 | 81.947 | 53.859 | 37.840 | 1.00 | 94.89 | | N |
| ANISOU | 1836 | NE2 | GLN | A | 238 | 10317 | 15085 | 10652 | −2518 | −41 | −2377 | N |
| ATOM | 1837 | C | GLN | A | 238 | 83.646 | 50.419 | 39.928 | 1.00 | 79.22 | | C |
| ANISOU | 1837 | C | GLN | A | 238 | 7978 | 14081 | 8043 | −1625 | −704 | −2512 | C |
| ATOM | 1838 | O | GLN | A | 238 | 84.198 | 50.410 | 38.818 | 1.00 | 79.96 | | O |
| ANISOU | 1838 | O | GLN | A | 238 | 7869 | 14314 | 8199 | −1673 | −683 | −2503 | O |
| ATOM | 1839 | N | SER | A | 239 | 83.991 | 49.579 | 40.939 | 1.00 | 75.57 | | N |
| ANISOU | 1839 | N | SER | A | 239 | 7514 | 13845 | 7354 | −1352 | −886 | −2563 | N |
| ATOM | 1840 | CA | SER | A | 239 | 85.024 | 48.542 | 40.816 | 1.00 | 75.79 | | C |
| ANISOU | 1840 | CA | SER | A | 239 | 7311 | 14303 | 7183 | −1054 | −1079 | −2606 | C |
| ATOM | 1841 | CB | SER | A | 239 | 85.364 | 47.946 | 42.178 | 1.00 | 80.04 | | C |
| ANISOU | 1841 | CB | SER | A | 239 | 7893 | 15066 | 7452 | −804 | −1259 | −2696 | C |
| ATOM | 1842 | OG | SER | A | 239 | 84.506 | 46.865 | 42.510 | 1.00 | 90.06 | | O |
| ANISOU | 1842 | OG | SER | A | 239 | 9467 | 16077 | 8674 | −453 | −1286 | −2503 | O |
| ATOM | 1843 | C | SER | A | 239 | 84.561 | 47.421 | 39.855 | 1.00 | 80.83 | | C |
| ANISOU | 1843 | C | SER | A | 239 | 8043 | 14779 | 7889 | −750 | −1077 | −2379 | C |
| ATOM | 1844 | O | SER | A | 239 | 85.351 | 46.988 | 39.013 | 1.00 | 81.76 | | O |
| ANISOU | 1844 | O | SER | A | 239 | 7929 | 15163 | 7973 | −651 | −1136 | −2388 | O |
| ATOM | 1845 | N | GLN | A | 240 | 83.282 | 46.966 | 39.979 | 1.00 | 75.54 | | N |
| ANISOU | 1845 | N | GLN | A | 240 | 7709 | 13683 | 7309 | −620 | −1001 | −2185 | N |
| ATOM | 1846 | CA | GLN | A | 240 | 82.672 | 45.905 | 39.152 | 1.00 | 73.98 | | C |
| ANISOU | 1846 | CA | GLN | A | 240 | 7653 | 13280 | 7174 | −365 | −983 | −1972 | C |
| ATOM | 1847 | CB | GLN | A | 240 | 81.613 | 45.146 | 39.973 | 1.00 | 74.79 | | C |
| ANISOU | 1847 | CB | GLN | A | 240 | 8111 | 13081 | 7224 | −156 | −977 | −1840 | C |
| ATOM | 1848 | CG | GLN | A | 240 | 82.154 | 44.493 | 41.244 | 1.00 | 77.61 | | C |
| ANISOU | 1848 | CG | GLN | A | 240 | 8525 | 13668 | 7295 | 98 | −1141 | −1926 | C |
| ATOM | 1849 | CD | GLN | A | 240 | 82.897 | 43.211 | 40.968 | 1.00 | 94.74 | | C |
| ANISOU | 1849 | CD | GLN | A | 240 | 10646 | 16088 | 9262 | 485 | −1292 | −1891 | C |
| ATOM | 1850 | OE1 | GLN | A | 240 | 84.128 | 43.137 | 41.103 | 1.00 | 87.74 | | O |
| ANISOU | 1850 | OE1 | GLN | A | 240 | 9495 | 15646 | 8198 | 587 | −1438 | −2038 | O |
| ATOM | 1851 | NE2 | GLN | A | 240 | 82.163 | 42.168 | 40.578 | 1.00 | 89.88 | | N |
| ANISOU | 1851 | NE2 | GLN | A | 240 | 10288 | 15204 | 8657 | 711 | −1256 | −1703 | N |
| ATOM | 1852 | C | GLN | A | 240 | 82.052 | 46.447 | 37.847 | 1.00 | 75.94 | | C |
| ANISOU | 1852 | C | GLN | A | 240 | 7900 | 13262 | 7691 | −573 | −810 | −1859 | C |
| ATOM | 1853 | O | GLN | A | 240 | 81.470 | 45.690 | 37.060 | 1.00 | 74.29 | | O |
| ANISOU | 1853 | O | GLN | A | 240 | 7795 | 12874 | 7556 | −410 | −780 | −1691 | O |
| ATOM | 1854 | N | LEU | A | 241 | 82.204 | 47.757 | 37.625 | 1.00 | 72.81 | | N |
| ANISOU | 1854 | N | LEU | A | 241 | 7399 | 12843 | 7422 | −931 | −697 | −1958 | N |
| ATOM | 1855 | CA | LEU | A | 241 | 81.640 | 48.479 | 36.496 | 1.00 | 73.17 | | C |
| ANISOU | 1855 | CA | LEU | A | 241 | 7467 | 12632 | 7703 | −1146 | −520 | −1867 | C |
| ATOM | 1856 | CB | LEU | A | 241 | 80.735 | 49.592 | 37.055 | 1.00 | 73.04 | | C |
| ANISOU | 1856 | CB | LEU | A | 241 | 7650 | 12298 | 7803 | −1399 | −368 | −1878 | C |
| ATOM | 1857 | CG | LEU | A | 241 | 79.435 | 49.877 | 36.320 | 1.00 | 77.07 | | C |
| ANISOU | 1857 | CG | LEU | A | 241 | 8367 | 12393 | 8523 | −1436 | −199 | −1697 | C |
| ATOM | 1858 | CD1 | LEU | A | 241 | 78.433 | 48.783 | 36.555 | 1.00 | 77.04 | | C |
| ANISOU | 1858 | CD1 | LEU | A | 241 | 8567 | 12199 | 8504 | −1163 | −233 | −1539 | C |
| ATOM | 1859 | CD2 | LEU | A | 241 | 78.846 | 51.176 | 36.788 | 1.00 | 78.00 | | C |
| ANISOU | 1859 | CD2 | LEU | A | 241 | 8632 | 12272 | 8732 | −1703 | −44 | −1746 | C |
| ATOM | I860 | C | LEU | A | 241 | 82.690 | 49.083 | 35.562 | 1.00 | 78.75 | | C |
| ANISOU | I860 | C | LEU | A | 241 | 7883 | 13593 | 8446 | −1344 | −492 | −1968 | C |
| ATOM | 1861 | O | LEU | A | 241 | 83.739 | 49.557 | 36.000 | 1.00 | 78.73 | | O |
| ANISOU | 1861 | O | LEU | A | 241 | 7666 | 13914 | 8333 | −1503 | −546 | −2167 | O |
| ATOM | 1862 | N | GLN | A | 242 | 82.373 | 49.090 | 34.271 | 1.00 | 76.56 | | N |
| ANISOU | 1862 | N | GLN | A | 242 | 7601 | 13168 | 8320 | −1351 | −396 | −1837 | N |
| ATOM | 1863 | CA | GLN | A | 242 | 83.193 | 49.657 | 33.192 | 1.00 | 77.25 | | C |
| ANISOU | 1863 | CA | GLN | A | 242 | 7457 | 13427 | 8469 | −1544 | −330 | −1896 | C |
| ATOM | 1864 | CB | GLN | A | 242 | 82.961 | 48.817 | 31.912 | 1.00 | 78.79 | | C |
| ANISOU | 1864 | CB | GLN | A | 242 | 7649 | 13550 | 8739 | −1334 | −325 | −1721 | C |
| ATOM | 1865 | CG | GLN | A | 242 | 83.654 | 49.254 | 30.626 | 1.00 | 88.74 | | C |
| ANISOU | 1865 | CG | GLN | A | 242 | 8700 | 14947 | 10069 | −1485 | −244 | −1740 | C |
| ATOM | 1866 | CD | GLN | A | 242 | 83.333 | 48.246 | 29.552 | 1.00 | 99.75 | | C |
| ANISOU | 1866 | CD | GLN | A | 242 | 10124 | 16269 | 11505 | −1219 | −269 | −1565 | C |
| ATOM | 1867 | OE1 | GLN | A | 242 | 82.234 | 48.233 | 28.982 | 1.00 | 90.97 | | O |
| ANISOU | 1867 | OE1 | GLN | A | 242 | 9216 | 14812 | 10537 | −1190 | −173 | −1402 | O |
| ATOM | 1868 | NE2 | GLN | A | 242 | 84.276 | 47.354 | 29.280 | 1.00 | 92.65 | | N |
| ANISOU | 1868 | NE2 | GLN | A | 242 | 9026 | 15709 | 10467 | −1002 | −403 | −1601 | N |
| ATOM | 1869 | C | GLN | A | 242 | 82.756 | 51.124 | 32.994 | 1.00 | 81.07 | | C |
| ANISOU | 1869 | C | GLN | A | 242 | 8049 | 13646 | 9109 | −1914 | −124 | −1924 | C |
| ATOM | 1870 | O | GLN | A | 242 | 83.598 | 52.014 | 32.797 | 1.00 | 80.48 | | O |
| ANISOU | 1870 | O | GLN | A | 242 | 7808 | 13741 | 9028 | −2206 | −58 | −2074 | O |
| ATOM | 1871 | N | GLY | A | 243 | 81.435 | 51.326 | 33.086 | 1.00 | 76.66 | | N |
| ANISOU | 1871 | N | GLY | A | 243 | 7776 | 12679 | 8672 | −1888 | −23 | −1783 | N |
| ATOM | 1872 | CA | GLY | A | 243 | 80.724 | 52.593 | 32.950 | 1.00 | 75.02 | | C |
| ANISOU | 1872 | CA | GLY | A | 243 | 7756 | 12145 | 8606 | −2146 | 177 | −1765 | C |
| ATOM | 1873 | C | GLY | A | 243 | 79.226 | 52.410 | 33.117 | 1.00 | 74.15 | | C |
| ANISOU | 1873 | C | GLY | A | 243 | 7924 | 11657 | 8591 | −1994 | 240 | −1592 | C |
| ATOM | 1874 | O | GLY | A | 243 | 78.728 | 51.278 | 33.182 | 1.00 | 73.25 | | O |
| ANISOU | 1874 | O | GLY | A | 243 | 7857 | 11523 | 8451 | −1719 | 141 | −1480 | O |
| ATOM | 1875 | N | PHE | A | 244 | 78.501 | 53.535 | 33.199 | 1.00 | 67.35 | | N |
| ANISOU | 1875 | N | PHE | A | 244 | 7259 | 10501 | 7830 | −2177 | 412 | −1577 | N |
| ATOM | 1876 | CA | PHE | A | 244 | 77.048 | 53.562 | 33.377 | 1.00 | 65.45 | | C |
| ANISOU | 1876 | CA | PHE | A | 244 | 7269 | 9921 | 7679 | −2058 | 495 | −1431 | C |
| ATOM | 1877 | CB | PHE | A | 244 | 76.668 | 53.514 | 34.877 | 1.00 | 66.77 | | C |
| ANISOU | 1877 | CB | PHE | A | 244 | 7569 | 10044 | 7757 | −2028 | 448 | −1496 | C |
| ATOM | 1878 | CG | PHE | A | 244 | 76.944 | 54.774 | 35.665 | 1.00 | 67.42 | | C |
| ANISOU | 1878 | CG | PHE | A | 244 | 7739 | 10071 | 7807 | −2300 | 542 | −1652 | C |
| ATOM | 1879 | CD2 | PHE | A | 244 | 75.930 | 55.691 | 35.915 | 1.00 | 68.83 | | C |
| ANISOU | 1879 | CD2 | PHE | A | 244 | 8167 | 9920 | 8064 | −2372 | 709 | −1605 | C |
| ATOM | 1880 | CE2 | PHE | A | 244 | 76.188 | 56.861 | 36.627 | 1.00 | 71.41 | | C |
| ANISOU | 1880 | CE2 | PHE | A | 244 | 8610 | 10172 | 8350 | −2627 | 807 | −1753 | C |
| ATOM | 1881 | CZ | PHE | A | 244 | 77.454 | 57.106 | 37.110 | 1.00 | 69.47 | | C |
| ANISOU | 1881 | CZ | PHE | A | 244 | 8208 | 10199 | 7987 | −2833 | 731 | −1959 | C |
| ATOM | 1882 | CE1 | PHE | A | 244 | 78.468 | 56.203 | 36.881 | 1.00 | 70.38 | | C |
| ANISOU | 1882 | CE1 | PHE | A | 244 | 8041 | 10679 | 8022 | −2760 | 557 | −2013 | C |
| ATOM | 1883 | CD1 | PHE | A | 244 | 78.212 | 55.035 | 36.167 | 1.00 | 69.70 | | C |
| ANISOU | 1883 | CD1 | PHE | A | 244 | 7861 | 10649 | 7973 | −2479 | 464 | −1854 | C |
| ATOM | 1884 | C | PHE | A | 244 | 76.404 | 54.749 | 32.665 | 1.00 | 66.69 | | C |
| ANISOU | 1884 | C | PHE | A | 244 | 7586 | 9784 | 7970 | −2208 | 707 | −1366 | C |
| ATOM | 1885 | O | PHE | A | 244 | 77.085 | 55.713 | 32.314 | 1.00 | 66.69 | | O |
| ANISOU | 1885 | O | PHE | A | 244 | 7558 | 9801 | 7980 | −2455 | 808 | −1459 | O |
| ATOM | 1886 | N | GLU | A | 245 | 75.091 | 54.691 | 32.484 | 1.00 | 61.38 | | N |
| ANISOU | 1886 | N | GLU | A | 245 | 7088 | 8849 | 7384 | −2059 | 780 | −1214 | N |
| ATOM | 1887 | CA | GLU | A | 245 | 74.354 | 55.720 | 31.779 | 1.00 | 61.02 | | C |
| ANISOU | 1887 | CA | GLU | A | 245 | 7213 | 8527 | 7447 | −2124 | 973 | −1130 | C |
| ATOM | 1888 | CB | GLU | A | 245 | 74.234 | 55.283 | 30.311 | 1.00 | 62.80 | | C |
| ANISOU | 1888 | CB | GLU | A | 245 | 7350 | 8758 | 7752 | −2009 | 983 | −994 | C |
| ATOM | 1889 | CG | GLU | A | 245 | 73.676 | 56.299 | 29.334 | 1.00 | 79.49 | | C |
| ANISOU | 1889 | CG | GLU | A | 245 | 9618 | 10626 | 9958 | −2049 | 1173 | −901 | C |
| ATOM | 1890 | CD | GLU | A | 245 | 73.486 | 55.730 | 27.937 | 1.00 | 113.16 | | C |
| ANISOU | 1890 | CD | GLU | A | 245 | 13793 | 14917 | 14287 | −1897 | 1159 | −763 | C |
| ATOM | 1891 | OE1 | GLU | A | 245 | 72.329 | 55.423 | 27.563 | 1.00 | 107.56 | | O |
| ANISOU | 1891 | OE1 | GLU | A | 245 | 13150 | 14088 | 13630 | −1696 | 1167 | −628 | O |
| ATOM | 1892 | OE2 | GLU | A | 245 | 74.502 | 55.570 | 27.220 | 1.00 | 115.07 | | O |
| ANISOU | 1892 | OE2 | GLU | A | 245 | 13884 | 15320 | 14518 | −1981 | 1136 | −797 | O |
| ATOM | 1893 | C | GLU | A | 245 | 72.989 | 55.866 | 32.423 | 1.00 | 63.53 | | C |
| ANISOU | 1893 | C | GLU | A | 245 | 7738 | 8607 | 7794 | −2001 | 1037 | −1050 | C |
| ATOM | 1894 | O | GLU | A | 245 | 72.256 | 54.884 | 32.534 | 1.00 | 62.14 | | O |
| ANISOU | 1894 | O | GLU | A | 245 | 7548 | 8437 | 7624 | −1791 | 957 | −954 | O |
| ATOM | 1895 | N | VAL | A | 246 | 72.662 | 57.081 | 32.887 | 1.00 | 60.68 | | N |
| ANISOU | 1895 | N | VAL | A | 246 | 7577 | 8039 | 7438 | −2138 | 1188 | −1098 | N |
| ATOM | 1896 | CA | VAL | A | 246 | 71.360 | 57.373 | 33.510 | 1.00 | 60.69 | | C |
| ANISOU | 1896 | CA | VAL | A | 246 | 7784 | 7816 | 7458 | −2022 | 1274 | −1032 | C |
| ATOM | 1897 | CB | VAL | A | 246 | 71.448 | 57.926 | 34.953 | 1.00 | 63.53 | | C |
| ANISOU | 1897 | CB | VAL | A | 246 | 8277 | 8131 | 7731 | −2147 | 1293 | −1170 | C |
| ATOM | 1898 | CG1 | VAL | A | 246 | 70.054 | 58.103 | 35.548 | 1.00 | 63.09 | | C |
| ANISOU | 1898 | CG1 | VAL | A | 246 | 8416 | 7864 | 7692 | −1996 | 1381 | −1091 | C |
| ATOM | 1899 | CG2 | VAL | A | 246 | 72.314 | 57.033 | 35.839 | 1.00 | 63.18 | | C |
| ANISOU | 1899 | CG2 | VAL | A | 246 | 8071 | 8355 | 7581 | −2162 | 1101 | −1283 | C |
| ATOM | 1900 | C | VAL | A | 246 | 70.586 | 58.301 | 32.582 | 1.00 | 66.23 | | C |
| ANISOU | 1900 | C | VAL | A | 246 | 8648 | 8273 | 8243 | −1984 | 1459 | −921 | C |
| ATOM | 1901 | O | VAL | A | 246 | 71.005 | 59.439 | 32.351 | 1.00 | 66.71 | | O |
| ANISOU | 1901 | O | VAL | A | 246 | 8841 | 8206 | 8301 | −2164 | 1599 | −978 | O |
| ATOM | 1902 | N | THR | A | 247 | 69.486 | 57.790 | 32.010 | 1.00 | 62.48 | | N |
| ANISOU | 1902 | N | THR | A | 247 | 8165 | 7746 | 7830 | −1750 | 1460 | −767 | N |
| ATOM | 1903 | CA | THR | A | 247 | 68.672 | 58.512 | 31.024 | 1.00 | 61.91 | | C |
| ANISOU | 1903 | CA | THR | A | 247 | 8220 | 7483 | 7820 | −1647 | 1611 | −646 | C |
| ATOM | 1904 | CB | THR | A | 247 | 68.765 | 57.818 | 29.618 | 1.00 | 66.88 | | C |
| ANISOU | 1904 | CB | THR | A | 247 | 8683 | 8222 | 8508 | −1539 | 1551 | −537 | C |
| ATOM | 1905 | OG1 | THR | A | 247 | 68.318 | 56.454 | 29.672 | 1.00 | 66.42 | | O |
| ANISOU | 1905 | OG1 | THR | A | 247 | 8522 | 8240 | 8474 | −1324 | 1458 | −440 | O |
| ATOM | 1906 | CG2 | THR | A | 247 | 70.159 | 57.876 | 29.000 | 1.00 | 62.44 | | C |
| ANISOU | 1906 | CG2 | THR | A | 247 | 7931 | 7866 | 7926 | −1683 | 1442 | −614 | C |
| ATOM | 1907 | C | THR | A | 247 | 67.215 | 58.594 | 31.456 | 1.00 | 64.93 | | C |
| ANISOU | 1907 | C | THR | A | 247 | 8720 | 7739 | 8212 | −1447 | 1673 | −564 | C |
| ATOM | 1908 | O | THR | A | 247 | 66.754 | 57.754 | 32.227 | 1.00 | 65.22 | | O |
| ANISOU | 1908 | O | THR | A | 247 | 8688 | 7865 | 8230 | −1364 | 1576 | −566 | O |
| ATOM | 1909 | N | GLY | A | 248 | 66.483 | 59.543 | 30.889 | 1.00 | 59.90 | | N |
| ANISOU | 1909 | N | GLY | A | 248 | 8256 | 6908 | 7594 | −1353 | 1834 | −485 | N |
| ATOM | 1910 | CA | GLY | A | 248 | 65.050 | 59.638 | 31.105 | 1.00 | 59.14 | | C |
| ANISOU | 1910 | CA | GLY | A | 248 | 8239 | 6731 | 7501 | −1128 | 1898 | −399 | C |
| ATOM | 1911 | C | GLY | A | 248 | 64.353 | 58.635 | 30.209 | 1.00 | 63.10 | | C |
| ANISOU | 1911 | C | GLY | A | 248 | 8546 | 7376 | 8053 | −933 | 1811 | −280 | C |
| ATOM | 1912 | O | GLY | A | 248 | 65.010 | 57.877 | 29.482 | 1.00 | 62.05 | | O |
| ANISOU | 1912 | O | GLY | A | 248 | 8246 | 7379 | 7950 | −973 | 1705 | −264 | O |
| ATOM | 1913 | N | ARG | A | 249 | 63.028 | 58.599 | 30.255 | 1.00 | 60.88 | | N |
| ANISOU | 1913 | N | ARG | A | 249 | 8278 | 7082 | 7773 | −727 | 1856 | −204 | N |
| ATOM | 1914 | CA | ARG | A | 249 | 62.308 | 57.675 | 29.395 | 1.00 | 61.80 | | C |
| ANISOU | 1914 | CA | ARG | A | 249 | 8206 | 7350 | 7926 | −563 | 1779 | −108 | C |
| ATOM | 1915 | CB | ARG | A | 249 | 61.400 | 56.722 | 30.175 | 1.00 | 62.86 | | C |
| ANISOU | 1915 | CB | ARG | A | 249 | 8233 | 7602 | 8047 | −492 | 1715 | −111 | C |
| ATOM | 1916 | CG | ARG | A | 249 | 61.211 | 55.344 | 29.525 | 1.00 | 75.10 | | C |
| ANISOU | 1916 | CG | ARG | A | 249 | 9565 | 9344 | 9626 | −455 | 1580 | −68 | C |
| ATOM | 1917 | CD | ARG | A | 249 | 59.754 | 55.037 | 29.200 | 1.00 | 93.26 | | C |
| ANISOU | 1917 | CD | ARG | A | 249 | 11772 | 11737 | 11927 | −273 | 1610 | 0 | C |
| ATOM | 1918 | NE | ARG | A | 249 | 58.925 | 54.946 | 30.407 | 1.00 | 111.82 | | N |
| ANISOU | 1918 | NE | ARG | A | 249 | 14153 | 14094 | 14238 | −258 | 1654 | −38 | N |
| ATOM | 1919 | CZ | ARG | A | 249 | 57.594 | 54.919 | 30.416 | 1.00 | 132.41 | | C |
| ANISOU | 1919 | CZ | ARG | A | 249 | 16692 | 16787 | 16830 | −112 | 1713 | −3 | C |
| ATOM | 1920 | NH1 | ARG | A | 249 | 56.933 | 54.856 | 31.568 | 1.00 | 121.46 | | N |
| ANISOU | 1920 | NH1 | ARG | A | 249 | 15338 | 15407 | 15403 | −117 | 1763 | −45 | N |
| ATOM | 1921 | NH2 | ARG | A | 249 | 56.913 | 54.960 | 29.276 | 1.00 | 119.49 | | N |
| ANISOU | 1921 | NH2 | ARG | A | 249 | 14944 | 15249 | 15206 | 42 | 1723 | 68 | N |
| ATOM | 1922 | C | ARG | A | 249 | 61.515 | 58.475 | 28.399 | 1.00 | 68.96 | | C |
| ANISOU | 1922 | C | ARG | A | 249 | 9190 | 8178 | 8834 | −365 | 1898 | −6 | C |
| ATOM | 1923 | O | ARG | A | 249 | 60.883 | 59.471 | 28.785 | 1.00 | 68.18 | | O |
| ANISOU | 1923 | O | ARG | A | 249 | 9273 | 7936 | 8694 | −262 | 2037 | 4 | O |
| ATOM | 1924 | N | PRO | A | 250 | 61.574 | 58.067 | 27.098 | 1.00 | 68.02 | | N |
| ANISOU | 1924 | N | PRO | A | 250 | 8946 | 8150 | 8747 | −293 | 1847 | 70 | N |
| ATOM | 1925 | CA | PRO | A | 250 | 60.831 | 58.798 | 26.061 | 1.00 | 67.86 | | C |
| ANISOU | 1925 | CA | PRO | A | 250 | 8997 | 8077 | 8710 | −71 | 1949 | 173 | C |
| ATOM | 1926 | CB | PRO | A | 250 | 61.250 | 58.071 | 24.786 | 1.00 | 69.85 | | C |
| ANISOU | 1926 | CB | PRO | A | 250 | 9079 | 8463 | 8999 | −64 | 1846 | 227 | C |
| ATOM | 1927 | CG | PRO | A | 250 | 61.618 | 56.661 | 25.227 | 1.00 | 74.21 | | C |
| ANISOU | 1927 | CG | PRO | A | 250 | 9427 | 9188 | 9584 | −199 | 1677 | 169 | C |
| ATOM | 1928 | CD | PRO | A | 250 | 62.311 | 56.914 | 26.517 | 1.00 | 69.85 | | C |
| ANISOU | 1928 | CD | PRO | A | 250 | 8980 | 8544 | 9018 | −386 | 1693 | 66 | C |
| ATOM | 1929 | C | PRO | A | 250 | 59.322 | 58.681 | 26.234 | 1.00 | 71.36 | | C |
| ANISOU | 1929 | C | PRO | A | 250 | 9375 | 8614 | 9125 | 163 | 1971 | 219 | C |
| ATOM | 1930 | O | PRO | A | 250 | 58.848 | 57.679 | 26.784 | 1.00 | 72.22 | | O |
| ANISOU | 1930 | O | PRO | A | 250 | 9314 | 8880 | 9248 | 138 | 1876 | 188 | O |
| ATOM | 1931 | N | LYS | A | 251 | 58.563 | 59.680 | 25.765 | 1.00 | 66.00 | | N |
| ANISOU | 1931 | N | LYS | A | 251 | 8832 | 7852 | 8395 | 393 | 2101 | 290 | N |
| ATOM | 1932 | CA | LYS | A | 251 | 57.110 | 59.564 | 25.837 | 1.00 | 65.21 | | C |
| ANISOU | 1932 | CA | LYS | A | 251 | 8627 | 7895 | 8253 | 638 | 2117 | 327 | C |
| ATOM | 1933 | CB | LYS | A | 251 | 56.423 | 60.953 | 25.878 | 1.00 | 67.42 | | C |
| ANISOU | 1933 | CB | LYS | A | 251 | 9153 | 8016 | 8448 | 883 | 2297 | 375 | C |
| ATOM | 1934 | CG | LYS | A | 251 | 55.059 | 60.988 | 26.599 | 1.00 | 76.13 | | C |
| ANISOU | 1934 | CG | LYS | A | 251 | 10178 | 9254 | 9496 | 1092 | 2336 | 371 | C |
| ATOM | 1935 | CD | LYS | A | 251 | 55.148 | 60.802 | 28.131 | 1.00 | 85.51 | | C |
| ANISOU | 1935 | CD | LYS | A | 251 | 11413 | 10385 | 10693 | 925 | 2352 | 276 | C |
| ATOM | 1936 | CE | LYS | A | 251 | 53.873 | 60.259 | 28.741 | 1.00 | 91.79 | | C |
| ANISOU | 1936 | CE | LYS | A | 251 | 12022 | 11398 | 11455 | 1060 | 2346 | 259 | C |
| ATOM | 1937 | C | LYS | A | 251 | 56.680 | 58.676 | 24.642 | 1.00 | 67.85 | | C |
| ANISOU | 1937 | C | LYS | A | 251 | 8710 | 8464 | 8605 | 747 | 2005 | 387 | C |
| ATOM | 1938 | O | LYS | A | 251 | 57.037 | 58.979 | 23.499 | 1.00 | 68.40 | | O |
| ANISOU | 1938 | O | LYS | A | 251 | 8818 | 8502 | 8668 | 823 | 2014 | 453 | O |
| ATOM | 1939 | N | HIS | A | 252 | 56.029 | 57.524 | 24.927 | 1.00 | 62.76 | | N |
| ANISOU | 1939 | N | HIS | A | 252 | 7821 | 8046 | 7978 | 721 | 1900 | 355 | N |
| ATOM | 1940 | CA | HIS | A | 252 | 55.593 | 56.539 | 23.928 | 1.00 | 62.37 | | C |
| ANISOU | 1940 | CA | HIS | A | 252 | 7525 | 8233 | 7938 | 778 | 1786 | 386 | C |
| ATOM | 1941 | CB | HIS | A | 252 | 55.510 | 55.118 | 24.539 | 1.00 | 63.59 | | C |
| ANISOU | 1941 | CB | HIS | A | 252 | 7492 | 8540 | 8128 | 585 | 1667 | 316 | C |
| ATOM | 1942 | CG | HIS | A | 252 | 56.840 | 54.498 | 24.893 | 1.00 | 67.46 | | C |
| ANISOU | 1942 | CG | HIS | A | 252 | 8029 | 8932 | 8670 | 335 | 1584 | 271 | C |
| ATOM | 1943 | ND1 | HIS | A | 252 | 57.045 | 53.129 | 24.794 | 1.00 | 69.44 | | N |
| ANISOU | 1943 | ND1 | HIS | A | 252 | 8133 | 9309 | 8942 | 202 | 1457 | 242 | N |
| ATOM | 1944 | CE1 | HIS | A | 252 | 58.297 | 52.923 | 25.181 | 1.00 | 68.64 | | C |
| ANISOU | 1944 | CE1 | HIS | A | 252 | 8116 | 9098 | 8865 | 34 | 1409 | 206 | C |
| ATOM | 1945 | NE2 | HIS | A | 252 | 58.900 | 54.064 | 25.527 | 1.00 | 68.91 | | N |
| ANISOU | 1945 | NE2 | HIS | A | 252 | 8325 | 8957 | 8900 | 20 | 1496 | 199 | N |
| ATOM | 1946 | CD2 | HIS | A | 252 | 57.981 | 55.072 | 25.359 | 1.00 | 69.28 | | C |
| ANISOU | 1946 | CD2 | HIS | A | 252 | 8435 | 8969 | 8918 | 207 | 1615 | 243 | C |
| ATOM | 1947 | C | HIS | A | 252 | 54.274 | 56.979 | 23.303 | 1.00 | 64.66 | | C |
| ANISOU | 1947 | C | HIS | A | 252 | 7733 | 8680 | 8156 | 1084 | 1838 | 442 | C |
| ATOM | 1948 | O | HIS | A | 252 | 53.238 | 56.370 | 23.532 | 1.00 | 62.90 | | O |
| ANISOU | 1948 | O | HIS | A | 252 | 7307 | 8685 | 7910 | 1136 | 1804 | 412 | O |
| ATOM | 1949 | N | LEU | A | 253 | 54.344 | 58.045 | 22.493 | 1.00 | 62.55 | | N |
| ANISOU | 1949 | N | LEU | A | 253 | 7629 | 8298 | 7839 | 1287 | 1925 | 520 | N |
| ATOM | 1950 | CA | LEU | A | 253 | 53.264 | 58.730 | 21.784 | 1.00 | 63.11 | | C |
| ANISOU | 1950 | CA | LEU | A | 253 | 7685 | 8483 | 7811 | 1638 | 1989 | 589 | C |
| ATOM | 1951 | CB | LEU | A | 253 | 53.831 | 59.699 | 20.752 | 1.00 | 63.32 | | C |
| ANISOU | 1951 | CB | LEU | A | 253 | 7943 | 8323 | 7793 | 1785 | 2067 | 682 | C |
| ATOM | 1952 | CG | LEU | A | 253 | 53.866 | 61.157 | 21.197 | 1.00 | 68.17 | | C |
| ANISOU | 1952 | CG | LEU | A | 253 | 8910 | 8654 | 8336 | 1926 | 2256 | 715 | C |
| ATOM | 1953 | CD1 | LEU | A | 253 | 55.013 | 61.420 | 22.190 | 1.00 | 68.29 | | C |
| ANISOU | 1953 | CD1 | LEU | A | 253 | 9126 | 8398 | 8421 | 1608 | 2306 | 647 | C |
| ATOM | 1954 | CD2 | LEU | A | 253 | 53.955 | 62.084 | 19.995 | 1.00 | 69.94 | | C |
| ANISOU | 1954 | CD2 | LEU | A | 253 | 9351 | 8748 | 8474 | 2160 | 2347 | 823 | C |
| ATOM | 1955 | C | LEU | A | 253 | 52.225 | 57.841 | 21.129 | 1.00 | 69.22 | | C |
| ANISOU | 1955 | C | LEU | A | 253 | 8132 | 9614 | 8557 | 1749 | 1880 | 582 | C |
| ATOM | 1956 | O | LEU | A | 253 | 51.049 | 58.193 | 21.200 | 1.00 | 69.22 | | O |
| ANISOU | 1956 | O | LEU | A | 253 | 8040 | 9789 | 8472 | 1992 | 1927 | 585 | O |
| ATOM | 1957 | N | TYR | A | 254 | 52.622 | 56.719 | 20.486 | 1.00 | 67.01 | | N |
| ANISOU | 1957 | N | TYR | A | 254 | 7674 | 9455 | 8332 | 1581 | 1742 | 566 | N |
| ATOM | 1958 | CA | TYR | A | 254 | 51.624 | 55.818 | 19.902 | 1.00 | 67.58 | | C |
| ANISOU | 1958 | CA | TYR | A | 254 | 7436 | 9871 | 8369 | 1646 | 1641 | 539 | C |
| ATOM | 1959 | CB | TYR | A | 254 | 52.228 | 54.837 | 18.884 | 1.00 | 67.71 | | C |
| ANISOU | 1959 | CB | TYR | A | 254 | 7336 | 9964 | 8426 | 1512 | 1507 | 544 | C |
| ATOM | I960 | CG | TYR | A | 254 | 51.179 | 54.085 | 18.085 | 1.00 | 67.90 | | C |
| ANISOU | I960 | CG | TYR | A | 254 | 7068 | 10343 | 8390 | 1610 | 1415 | 517 | C |
| ATOM | 1961 | CD1 | TYR | A | 254 | 50.246 | 54.764 | 17.310 | 1.00 | 70.34 | | C |
| ANISOU | 1961 | CD1 | TYR | A | 254 | 7305 | 10834 | 8588 | 1946 | 1443 | 563 | C |
| ATOM | 1962 | CE1 | TYR | A | 254 | 49.289 | 54.080 | 16.559 | 1.00 | 71.36 | | C |
| ANISOU | 1962 | CE1 | TYR | A | 254 | 7141 | 11325 | 8647 | 2027 | 1349 | 522 | C |
| ATOM | 1963 | CZ | TYR | A | 254 | 49.263 | 52.696 | 16.571 | 1.00 | 74.45 | | C |
| ANISOU | 1963 | CZ | TYR | A | 254 | 7333 | 11869 | 9084 | 1745 | 1240 | 433 | C |
| ATOM | 1964 | OH | TYR | A | 254 | 48.310 | 52.042 | 15.821 | 1.00 | 72.92 | | O |
| ANISOU | 1964 | OH | TYR | A | 254 | 6853 | 12040 | 8813 | 1796 | 1154 | 375 | O |
| ATOM | 1965 | CE2 | TYR | A | 254 | 50.188 | 51.997 | 17.332 | 1.00 | 67.82 | | C |
| ANISOU | 1965 | CE2 | TYR | A | 254 | 6598 | 10819 | 8352 | 1421 | 1222 | 397 | C |
| ATOM | 1966 | CD2 | TYR | A | 254 | 51.148 | 52.694 | 18.068 | 1.00 | 67.51 | | C |
| ANISOU | 1966 | CD2 | TYR | A | 254 | 6826 | 10448 | 8377 | 1368 | 1302 | 440 | C |
| ATOM | 1967 | C | TYR | A | 254 | 50.870 | 55.067 | 21.012 | 1.00 | 76.00 | | C |
| ANISOU | 1967 | C | TYR | A | 254 | 8331 | 11099 | 9446 | 1507 | 1630 | 444 | C |
| ATOM | 1968 | O | TYR | A | 254 | 49.640 | 55.026 | 20.978 | 1.00 | 76.42 | | O |
| ANISOU | 1968 | O | TYR | A | 254 | 8196 | 11416 | 9426 | 1671 | 1644 | 419 | O |
| ATOM | 1969 | N | SER | A | 255 | 51.612 | 54.520 | 22.012 | 1.00 | 74.40 | | N |
| ANISOU | 1969 | N | SER | A | 255 | 8205 | 10741 | 9321 | 1220 | 1614 | 389 | N |
| ATOM | 1970 | CA | SER | A | 255 | 51.093 | 53.784 | 23.176 | 1.00 | 74.69 | | C |
| ANISOU | 1970 | CA | SER | A | 255 | 8140 | 10871 | 9368 | 1052 | 1618 | 303 | C |
| ATOM | 1971 | CB | SER | A | 255 | 52.237 | 53.138 | 23.961 | 1.00 | 79.10 | | C |
| ANISOU | 1971 | CB | SER | A | 255 | 8821 | 11232 | 10001 | 756 | 1572 | 264 | C |
| ATOM | 1972 | OG | SER | A | 255 | 53.013 | 52.240 | 23.181 | 1.00 | 90.21 | | O |
| ANISOU | 1972 | OG | SER | A | 255 | 10189 | 12638 | 11449 | 622 | 1454 | 272 | O |
| ATOM | 1973 | C | SER | A | 255 | 50.295 | 54.717 | 24.093 | 1.00 | 79.32 | | C |
| ANISOU | 1973 | C | SER | A | 255 | 8792 | 11448 | 9899 | 1216 | 1752 | 294 | C |
| ATOM | 1974 | O | SER | A | 255 | 49.496 | 54.243 | 24.902 | 1.00 | 79.79 | | O |
| ANISOU | 1974 | O | SER | A | 255 | 8720 | 11660 | 9936 | 1153 | 1777 | 227 | O |
| ATOM | 1975 | N | ILE | A | 256 | 50.527 | 56.040 | 23.977 | 1.00 | 75.48 | | N |
| ANISOU | 1975 | N | ILE | A | 256 | 8528 | 10769 | 9381 | 1421 | 1850 | 359 | N |
| ATOM | 1976 | CA | ILE | A | 256 | 49.849 | 57.084 | 24.747 | 1.00 | 75.03 | | C |
| ANISOU | 1976 | CA | ILE | A | 256 | 8593 | 10660 | 9256 | 1624 | 1991 | 363 | C |
| ATOM | 1977 | CB | ILE | A | 256 | 50.823 | 58.280 | 25.019 | 1.00 | 77.11 | | C |
| ANISOU | 1977 | CB | ILE | A | 256 | 9223 | 10548 | 9526 | 1647 | 2091 | 409 | C |
| ATOM | 1978 | CG1 | ILE | A | 256 | 51.912 | 57.927 | 26.089 | 1.00 | 77.51 | | C |
| ANISOU | 1978 | CG1 | ILE | A | 256 | 9411 | 10378 | 9660 | 1313 | 2074 | 346 | C |
| ATOM | 1979 | CD1 | ILE | A | 256 | 51.459 | 57.535 | 27.595 | 1.00 | 86.06 | | C |
| ANISOU | 1979 | CD1 | ILE | A | 256 | 10454 | 11503 | 10742 | 1182 | 2102 | 261 | C |
| ATOM | 1980 | CG2 | ILE | A | 256 | 50.125 | 59.597 | 25.342 | 1.00 | 76.02 | | C |
| ANISOU | 1980 | CG2 | ILE | A | 256 | 9268 | 10329 | 9285 | 1956 | 2250 | 446 | C |
| ATOM | 1981 | C | ILE | A | 256 | 48.557 | 57.465 | 24.007 | 1.00 | 81.91 | | C |
| ANISOU | 1981 | C | ILE | A | 256 | 9288 | 11816 | 10018 | 1971 | 2018 | 394 | C |
| ATOM | 1982 | O | ILE | A | 256 | 47.548 | 57.725 | 24.645 | 1.00 | 81.71 | | O |
| ANISOU | 1982 | O | ILE | A | 256 | 9187 | 11936 | 9924 | 2118 | 2095 | 363 | O |
| ATOM | 1983 | N | TRP | A | 257 | 48.566 | 57.421 | 22.674 | 1.00 | 81.09 | | N |
| ANISOU | 1983 | N | TRP | A | 257 | 9100 | 11823 | 9888 | 2103 | 1948 | 448 | N |
| ATOM | 1984 | CA | TRP | A | 257 | 47.385 | 57.745 | 21.887 | 1.00 | 82.42 | | C |
| ANISOU | 1984 | CA | TRP | A | 257 | 9086 | 12297 | 9935 | 2451 | 1953 | 473 | C |
| ATOM | 1985 | CB | TRP | A | 257 | 47.803 | 58.367 | 20.563 | 1.00 | 81.62 | | C |
| ANISOU | 1985 | CB | TRP | A | 257 | 9114 | 12111 | 9787 | 2668 | 1940 | 575 | C |
| ATOM | 1986 | CG | TRP | A | 257 | 46.675 | 58.735 | 19.656 | 1.00 | 83.02 | | C |
| ANISOU | 1986 | CG | TRP | A | 257 | 9118 | 12609 | 9818 | 3061 | 1932 | 607 | C |
| ATOM | 1987 | CD1 | TRP | A | 257 | 45.893 | 59.851 | 19.721 | 1.00 | 86.02 | | C |
| ANISOU | 1987 | CD1 | TRP | A | 257 | 9584 | 13042 | 10059 | 3459 | 2046 | 649 | C |
| ATOM | 1988 | NE1 | TRP | A | 257 | 44.997 | 59.862 | 18.676 | 1.00 | 85.63 | | N |
| ANISOU | 1988 | NE1 | TRP | A | 257 | 9310 | 13347 | 9879 | 3776 | 1986 | 667 | N |
| ATOM | 1989 | CE2 | TRP | A | 257 | 45.213 | 58.756 | 17.895 | 1.00 | 87.21 | | C |
| ANISOU | 1989 | CE2 | TRP | A | 257 | 9281 | 13717 | 10140 | 3562 | 1833 | 632 | C |
| ATOM | 1990 | CD2 | TRP | A | 257 | 46.270 | 58.027 | 18.482 | 1.00 | 83.14 | | C |
| ANISOU | 1990 | CD2 | TRP | A | 257 | 8857 | 12940 | 9793 | 3118 | 1800 | 598 | C |
| ATOM | 1991 | CE3 | TRP | A | 257 | 46.695 | 56.836 | 17.870 | 1.00 | 84.45 | | C |
| ANISOU | 1991 | CE3 | TRP | A | 257 | 8852 | 13199 | 10037 | 2846 | 1656 | 561 | C |
| ATOM | 1992 | CZ3 | TRP | A | 257 | 46.054 | 56.411 | 16.723 | 1.00 | 86.04 | | C |
| ANISOU | 1992 | CZ3 | TRP | A | 257 | 8797 | 13740 | 10153 | 2994 | 1551 | 552 | C |
| ATOM | 1993 | CH2 | TRP | A | 257 | 45.001 | 57.151 | 16.166 | 1.00 | 86.81 | | C |
| ANISOU | 1993 | CH2 | TRP | A | 257 | 8791 | 14111 | 10083 | 3426 | 1578 | 579 | C |
| ATOM | 1994 | CZ2 | TRP | A | 257 | 44.571 | 58.330 | 16.730 | 1.00 | 86.71 | | C |
| ANISOU | 1994 | CZ2 | TRP | A | 257 | 8943 | 14020 | 9983 | 3729 | 1718 | 623 | C |
| ATOM | 1995 | C | TRP | A | 257 | 46.433 | 56.545 | 21.713 | 1.00 | 89.36 | | C |
| ANISOU | 1995 | C | TRP | A | 257 | 9560 | 13594 | 10798 | 2358 | 1851 | 385 | C |
| ATOM | 1996 | O | TRP | A | 257 | 45.220 | 56.753 | 21.628 | 1.00 | 89.46 | | O |
| ANISOU | 1996 | O | TRP | A | 257 | 9365 | 13925 | 10701 | 2604 | 1876 | 360 | O |
| ATOM | 1997 | N | LYS | A | 258 | 46.978 | 55.300 | 21.696 | 1.00 | 87.69 | | N |
| ANISOU | 1997 | N | LYS | A | 258 | 9245 | 13388 | 10683 | 2001 | 1743 | 330 | N |
| ATOM | 1998 | CA | LYS | A | 258 | 46.234 | 54.022 | 21.616 | 1.00 | 88.13 | | C |
| ANISOU | 1998 | CA | LYS | A | 258 | 8970 | 13781 | 10734 | 1813 | 1656 | 231 | C |
| ATOM | 1999 | CB | LYS | A | 258 | 47.207 | 52.820 | 21.776 | 1.00 | 90.70 | | C |
| ANISOU | 1999 | CB | LYS | A | 258 | 9343 | 13947 | 11170 | 1419 | 1568 | 195 | C |
| ATOM | 2000 | CG | LYS | A | 258 | 47.787 | 52.213 | 20.495 | 1.00 | 100.69 | | C |
| ANISOU | 2000 | CG | LYS | A | 258 | 10563 | 15236 | 12458 | 1361 | 1442 | 221 | C |
| ATOM | 2001 | CD | LYS | A | 258 | 49.144 | 51.531 | 20.783 | 1.00 | 107.64 | | C |
| ANISOU | 2001 | CD | LYS | A | 258 | 11626 | 15830 | 13444 | 1066 | 1388 | 225 | C |
| ATOM | 2002 | CE | LYS | A | 258 | 49.128 | 50.023 | 20.929 | 1.00 | 110.52 | | C |
| ANISOU | 2002 | CE | LYS | A | 258 | 11864 | 16297 | 13831 | 760 | 1307 | 140 | C |
| ATOM | 2003 | NZ | LYS | A | 258 | 50.473 | 49.490 | 21.300 | 1.00 | 111.28 | | N |
| ANISOU | 2003 | NZ | LYS | A | 258 | 12165 | 16111 | 14008 | 535 | 1265 | 150 | N |
| ATOM | 2004 | C | LYS | A | 258 | 45.207 | 53.972 | 22.774 | 1.00 | 92.58 | | C |
| ANISOU | 2004 | C | LYS | A | 258 | 9397 | 14524 | 11255 | 1794 | 1745 | 149 | C |
| ATOM | 2005 | O | LYS | A | 258 | 44.021 | 53.686 | 22.554 | 1.00 | 91.63 | | O |
| ANISOU | 2005 | O | LYS | A | 258 | 8971 | 14794 | 11051 | 1872 | 1736 | 81 | O |
| ATOM | 2006 | N | LYS | A | 259 | 45.704 | 54.273 | 24.005 | 1.00 | 89.54 | | N |
| ANISOU | 2006 | N | LYS | A | 259 | 9238 | 13863 | 10922 | 1682 | 1832 | 149 | N |
| ATOM | 2007 | CA | LYS | A | 259 | 44.978 | 54.307 | 25.270 | 1.00 | 89.50 | | C |
| ANISOU | 2007 | CA | LYS | A | 259 | 9186 | 13934 | 10887 | 1643 | 1935 | 82 | C |
| ATOM | 2008 | CB | LYS | A | 259 | 45.976 | 54.450 | 26.440 | 1.00 | 90.94 | | C |
| ANISOU | 2008 | CB | LYS | A | 259 | 9675 | 13731 | 11147 | 1452 | 1988 | 90 | C |
| ATOM | 2009 | CG | LYS | A | 259 | 45.485 | 53.877 | 27.768 | 1.00 | 98.33 | | C |
| ANISOU | 2009 | CG | LYS | A | 259 | 10554 | 14725 | 12081 | 1241 | 2049 | 3 | C |
| ATOM | 2010 | CD | LYS | A | 259 | 46.346 | 54.336 | 28.949 | 1.00 | 103.93 | | C |
| ANISOU | 2010 | CD | LYS | A | 259 | 11577 | 15081 | 12833 | 1148 | 2117 | 13 | C |
| ATOM | 2011 | CE | LYS | A | 259 | 45.827 | 53.880 | 30.298 | 1.00 | 109.83 | | C |
| ANISOU | 2011 | CE | LYS | A | 259 | 12285 | 15885 | 13559 | 975 | 2192 | −66 | C |
| ATOM | 2012 | NZ | LYS | A | 259 | 46.545 | 54.533 | 31.431 | 1.00 | 113.22 | | N |
| ANISOU | 2012 | NZ | LYS | A | 259 | 13014 | 16001 | 14002 | 942 | 2267 | −61 | N |
| ATOM | 2013 | C | LYS | A | 259 | 43.942 | 55.450 | 25.267 | 1.00 | 95.67 | | C |
| ANISOU | 2013 | C | LYS | A | 259 | 9910 | 14894 | 11546 | 2047 | 2038 | 105 | C |
| ATOM | 2014 | O | LYS | A | 259 | 42.771 | 55.213 | 25.580 | 1.00 | 96.23 | | O |
| ANISOU | 2014 | O | LYS | A | 259 | 9708 | 15315 | 11540 | 2105 | 2074 | 30 | O |
| ATOM | 2015 | N | MET | A | 260 | 44.369 | 56.668 | 24.871 | 1.00 | 92.47 | | N |
| ANISOU | 2015 | N | MET | A | 260 | 9765 | 14258 | 11110 | 2330 | 2090 | 205 | N |
| ATOM | 2016 | CA | MET | A | 260 | 43.570 | 57.900 | 24.803 | 1.00 | 92.34 | | C |
| ANISOU | 2016 | CA | MET | A | 260 | 9800 | 14321 | 10962 | 2770 | 2197 | 252 | C |
| ATOM | 2017 | CB | MET | A | 260 | 44.440 | 58.997 | 24.228 | 1.00 | 94.31 | | C |
| ANISOU | 2017 | CB | MET | A | 260 | 10414 | 14213 | 11206 | 2958 | 2236 | 369 | C |
| ATOM | 2018 | CG | MET | A | 260 | 44.256 | 60.310 | 24.877 | 1.00 | 97.54 | | C |
| ANISOU | 2018 | CG | MET | A | 260 | 11106 | 14429 | 11527 | 3250 | 2397 | 415 | C |
| ATOM | 2019 | SD | MET | A | 260 | 45.214 | 61.484 | 23.914 | 1.00 | 101.34 | | S |
| ANISOU | 2019 | SD | MET | A | 260 | 12000 | 14526 | 11978 | 3457 | 2446 | 549 | S |
| ATOM | 2020 | CE | MET | A | 260 | 46.863 | 61.029 | 24.382 | 1.00 | 97.96 | | C |
| ANISOU | 2020 | CE | MET | A | 260 | 11806 | 13687 | 11728 | 2966 | 2414 | 530 | C |
| ATOM | 2021 | C | MET | A | 260 | 42.335 | 57.778 | 23.926 | 1.00 | 97.78 | | C |
| ANISOU | 2021 | C | MET | A | 260 | 10136 | 15490 | 11525 | 3044 | 2152 | 229 | C |
| ATOM | 2022 | O | MET | A | 260 | 41.311 | 58.406 | 24.209 | 1.00 | 96.69 | | O |
| ANISOU | 2022 | O | MET | A | 260 | 9911 | 15566 | 11261 | 3365 | 2240 | 219 | O |
| ATOM | 2023 | N | GLU | A | 261 | 42.463 | 57.014 | 22.830 | 1.00 | 96.47 | | N |
| ANISOU | 2023 | N | GLU | A | 261 | 9775 | 15498 | 11383 | 2938 | 2014 | 218 | N |
| ATOM | 2024 | CA | GLU | A | 261 | 41.408 | 56.766 | 21.851 | 1.00 | 97.29 | | C |
| ANISOU | 2024 | CA | GLU | A | 261 | 9520 | 16080 | 11366 | 3154 | 1941 | 180 | C |
| ATOM | 2025 | CB | GLU | A | 261 | 42.034 | 56.392 | 20.495 | 1.00 | 98.58 | | C |
| ANISOU | 2025 | CB | GLU | A | 261 | 9680 | 16221 | 11554 | 3125 | 1807 | 229 | C |
| ATOM | 2026 | CG | GLU | A | 261 | 41.401 | 57.095 | 19.306 | 1.00 | 109.47 | | C |
| ANISOU | 2026 | CG | GLU | A | 261 | 10966 | 17855 | 12773 | 3586 | 1778 | 286 | C |
| ATOM | 2027 | CD | GLU | A | 261 | 41.584 | 58.601 | 19.217 | 1.00 | 133.75 | | C |
| ANISOU | 2027 | CD | GLU | A | 261 | 14391 | 20671 | 15757 | 4014 | 1898 | 415 | C |
| ATOM | 2028 | OE1 | GLU | A | 261 | 40.774 | 59.249 | 18.516 | 1.00 | 129.65 | | O |
| ANISOU | 2028 | OE1 | GLU | A | 261 | 13784 | 20411 | 15066 | 4458 | 1900 | 452 | O |
| ATOM | 2029 | OE2 | GLU | A | 261 | 42.533 | 59.134 | 19.837 | 1.00 | 130.14 | | O |
| ANISOU | 2029 | OE2 | GLU | A | 261 | 14305 | 19757 | 15386 | 3912 | 1992 | 475 | O |
| ATOM | 2030 | C | GLU | A | 261 | 40.436 | 55.670 | 22.315 | 1.00 | 103.26 | | C |
| ANISOU | 2030 | C | GLU | A | 261 | 9873 | 17253 | 12107 | 2917 | 1916 | 32 | C |
| ATOM | 2031 | O | GLU | A | 261 | 39.258 | 55.703 | 21.941 | 1.00 | 102.87 | | O |
| ANISOU | 2031 | O | GLU | A | 261 | 9501 | 17663 | 11924 | 3144 | 1911 | −28 | O |
| ATOM | 2032 | N | ARG | A | 262 | 40.931 | 54.713 | 23.130 | 1.00 | 100.81 | | N |
| ANISOU | 2032 | N | ARG | A | 262 | 9592 | 16790 | 11922 | 2464 | 1906 | −31 | N |
| ATOM | 2033 | CA | ARG | A | 262 | 40.155 | 53.581 | 23.627 | 1.00 | 101.12 | | C |
| ANISOU | 2033 | CA | ARG | A | 262 | 9319 | 17146 | 11957 | 2157 | 1902 | −172 | C |
| ATOM | 2034 | C | ARG | A | 262 | 39.479 | 53.839 | 24.998 | 1.00 | 106.70 | | C |
| ANISOU | 2034 | C | ARG | A | 262 | 9997 | 17913 | 12630 | 2151 | 2048 | −229 | C |
| ATOM | 2035 | O | ARG | A | 262 | 38.248 | 53.841 | 25.059 | 1.00 | 106.60 | | O |
| ANISOU | 2035 | O | ARG | A | 262 | 9655 | 18341 | 12506 | 2263 | 2089 | −317 | O |
| ATOM | 2036 | CB | ARG | A | 262 | 41.031 | 52.313 | 23.637 | 1.00 | 100.66 | | C |
| ANISOU | 2036 | CB | ARG | A | 262 | 9334 | 16887 | 12024 | 1692 | 1818 | −205 | C |
| ATOM | 2037 | CG | ARG | A | 262 | 40.397 | 51.070 | 24.264 | 1.00 | 109.42 | | C |
| ANISOU | 2037 | CG | ARG | A | 262 | 10218 | 18223 | 13134 | 1314 | 1841 | −346 | C |
| ATOM | 2038 | CD | ARG | A | 262 | 41.422 | 49.970 | 24.498 | 1.00 | 119.36 | | C |
| ANISOU | 2038 | CD | ARG | A | 262 | 11669 | 19176 | 14507 | 898 | 1789 | −356 | C |
| ATOM | 2039 | NE | ARG | A | 262 | 42.512 | 50.392 | 25.388 | 1.00 | 125.31 | | N |
| ANISOU | 2039 | NE | ARG | A | 262 | 12796 | 19468 | 15350 | 865 | 1835 | −273 | N |
| ATOM | 2040 | CZ | ARG | A | 262 | 43.794 | 50.457 | 25.036 | 1.00 | 134.69 | | C |
| ANISOU | 2040 | CZ | ARG | A | 262 | 14242 | 20310 | 16622 | 828 | 1760 | −189 | C |
| ATOM | 2041 | NH1 | ARG | A | 262 | 44.707 | 50.866 | 25.905 | 1.00 | 116.40 | | N |
| ANISOU | 2041 | NH1 | ARG | A | 262 | 12230 | 17627 | 14371 | 787 | 1805 | −136 | N |
| ATOM | 2042 | NH2 | ARG | A | 262 | 44.172 | 50.124 | 23.804 | 1.00 | 120.28 | | N |
| ANISOU | 2042 | NH2 | ARG | A | 262 | 12366 | 18525 | 14810 | 827 | 1642 | −165 | N |
| ATOM | 2043 | N | GLU | A | 263 | 40.242 | 54.135 | 26.038 | 1.00 | 104.29 | | N |
| ANISOU | 2043 | N | GLU | A | 263 | 10016 | 17197 | 12411 | 2015 | 2125 | −190 | N |
| ATOM | 2044 | CA | GLU | A | 263 | 39.566 | 54.309 | 27.343 | 1.00 | 104.80 | | C |
| ANISOU | 2044 | CA | GLU | A | 263 | 10087 | 17284 | 12448 | 1976 | 2265 | −244 | C |
| ATOM | 2045 | CB | GLU | A | 263 | 40.097 | 53.283 | 28.333 | 1.00 | 106.38 | | C |
| ANISOU | 2045 | CB | GLU | A | 263 | 10415 | 17253 | 12754 | 1510 | 2274 | −294 | C |
| ATOM | 2046 | CG | GLU | A | 263 | 41.444 | 53.643 | 28.904 | 1.00 | 117.94 | | C |
| ANISOU | 2046 | CG | GLU | A | 263 | 12270 | 18208 | 14333 | 1378 | 2231 | −210 | C |
| ATOM | 2047 | CD | GLU | A | 263 | 42.051 | 52.425 | 29.559 | 1.00 | 143.87 | | C |
| ANISOU | 2047 | CD | GLU | A | 263 | 15714 | 21271 | 17680 | 1022 | 2269 | −254 | C |
| ATOM | 2048 | OE1 | GLU | A | 263 | 42.882 | 52.574 | 30.462 | 1.00 | 141.37 | | O |
| ANISOU | 2048 | OE1 | GLU | A | 263 | 15615 | 20737 | 17362 | 1076 | 2365 | −236 | O |
| ATOM | 2049 | OE2 | GLU | A | 263 | 41.663 | 51.330 | 29.170 | 1.00 | 140.02 | | O |
| ANISOU | 2049 | OE2 | GLU | A | 263 | 15152 | 20822 | 17229 | 700 | 2205 | −307 | O |
| ATOM | 2050 | C | GLU | A | 263 | 39.838 | 55.685 | 27.921 | 1.00 | 108.98 | | C |
| ANISOU | 2050 | C | GLU | A | 263 | 10894 | 17581 | 12934 | 2322 | 2383 | −162 | C |
| ATOM | 2051 | O | GLU | A | 263 | 39.689 | 55.848 | 29.127 | 1.00 | 108.05 | | O |
| ANISOU | 2051 | O | GLU | A | 263 | 10786 | 17491 | 12776 | 2342 | 2507 | −203 | O |
| ATOM | 2052 | N | GLY | A | 264 | 40.194 | 56.643 | 27.091 | 1.00 | 106.22 | | N |
| ANISOU | 2052 | N | GLY | A | 264 | 10781 | 16996 | 12583 | 2580 | 2356 | −50 | N |
| ATOM | 2053 | CA | GLY | A | 264 | 40.590 | 57.925 | 27.686 | 1.00 | 106.30 | | C |
| ANISOU | 2053 | CA | GLY | A | 264 | 11115 | 16731 | 12544 | 2890 | 2475 | 29 | C |
| ATOM | 2054 | C | GLY | A | 264 | 39.967 | 59.110 | 27.007 | 1.00 | 110.44 | | C |
| ANISOU | 2054 | C | GLY | A | 264 | 11601 | 17448 | 12914 | 3412 | 2517 | 88 | C |
| ATOM | 2055 | O | GLY | A | 264 | 39.105 | 58.925 | 26.153 | 1.00 | 110.01 | | O |
| ANISOU | 2055 | O | GLY | A | 264 | 11193 | 17839 | 12767 | 3574 | 2458 | 49 | O |
| ATOM | 2056 | N | LYS | A | 265 | 40.299 | 60.288 | 27.504 | 1.00 | 106.98 | | N |
| ANISOU | 2056 | N | LYS | A | 265 | 11549 | 16666 | 12434 | 3677 | 2624 | 178 | N |
| ATOM | 2057 | CA | LYS | A | 265 | 39.745 | 61.494 | 26.872 | 1.00 | 106.62 | | C |
| ANISOU | 2057 | CA | LYS | A | 265 | 11615 | 16671 | 12225 | 4214 | 2697 | 259 | C |
| ATOM | 2058 | CB | LYS | A | 265 | 38.767 | 62.177 | 27.832 | 1.00 | 109.42 | | C |
| ANISOU | 2058 | CB | LYS | A | 265 | 11961 | 17167 | 12447 | 4510 | 2851 | 227 | C |
| ATOM | 2059 | C | LYS | A | 265 | 40.890 | 62.394 | 26.417 | 1.00 | 108.25 | | C |
| ANISOU | 2059 | C | LYS | A | 265 | 12303 | 16367 | 12459 | 4295 | 2732 | 379 | C |
| ATOM | 2060 | O | LYS | A | 265 | 40.872 | 62.805 | 25.270 | 1.00 | 108.03 | | O |
| ANISOU | 2060 | O | LYS | A | 265 | 12299 | 16355 | 12392 | 4468 | 2667 | 452 | O |
| ATOM | 2061 | N | THR | A | 266 | 41.855 | 62.670 | 27.278 | 1.00 | 102.17 | | N |
| ANISOU | 2061 | N | THR | A | 266 | 11912 | 15156 | 11750 | 4144 | 2834 | 393 | N |
| ATOM | 2062 | CA | THR | A | 266 | 42.904 | 63.584 | 26.802 | 1.00 | 100.85 | | C |
| ANISOU | 2062 | CA | THR | A | 266 | 12212 | 14503 | 11605 | 4168 | 2889 | 487 | C |
| ATOM | 2063 | CB | THR | A | 266 | 42.423 | 65.005 | 27.018 | 1.00 | 107.62 | | C |
| ANISOU | 2063 | CB | THR | A | 266 | 13437 | 15151 | 12304 | 4588 | 3077 | 547 | C |
| ATOM | 2064 | OG1 | THR | A | 266 | 43.578 | 65.819 | 26.872 | 1.00 | 107.83 | | O |
| ANISOU | 2064 | OG1 | THR | A | 266 | 13929 | 14694 | 12346 | 4562 | 3144 | 628 | O |
| ATOM | 2065 | CG2 | THR | A | 266 | 41.856 | 65.160 | 28.403 | 1.00 | 105.43 | | C |
| ANISOU | 2065 | CG2 | THR | A | 266 | 13220 | 14832 | 12008 | 4550 | 3194 | 476 | C |
| ATOM | 2066 | C | THR | A | 266 | 44.198 | 63.364 | 27.575 | 1.00 | 102.34 | | C |
| ANISOU | 2066 | C | THR | A | 266 | 12590 | 14326 | 11967 | 3673 | 2854 | 455 | C |
| ATOM | 2067 | O | THR | A | 266 | 44.121 | 63.063 | 28.761 | 1.00 | 101.59 | | O |
| ANISOU | 2067 | O | THR | A | 266 | 12362 | 14285 | 11952 | 3375 | 2829 | 368 | O |
| ATOM | 2068 | N | LEU | A | 267 | 45.308 | 63.754 | 26.959 | 1.00 | 96.64 | | N |
| ANISOU | 2068 | N | LEU | A | 267 | 12185 | 13244 | 11289 | 3601 | 2858 | 524 | N |
| ATOM | 2069 | CA | LEU | A | 267 | 46.668 | 63.532 | 27.482 | 1.00 | 95.21 | | C |
| ANISOU | 2069 | CA | LEU | A | 267 | 12214 | 12715 | 11248 | 3181 | 2832 | 498 | C |
| ATOM | 2070 | CB | LEU | A | 267 | 47.650 | 64.231 | 26.546 | 1.00 | 94.83 | | C |
| ANISOU | 2070 | CB | LEU | A | 267 | 12447 | 12375 | 11208 | 3187 | 2844 | 582 | C |
| ATOM | 2071 | C | LEU | A | 267 | 46.723 | 64.174 | 28.846 | 1.00 | 98.79 | | C |
| ANISOU | 2071 | C | LEU | A | 267 | 12945 | 12908 | 11682 | 3120 | 2967 | 452 | C |
| ATOM | 2072 | O | LEU | A | 267 | 47.481 | 63.698 | 29.677 | 1.00 | 98.16 | | O |
| ANISOU | 2072 | O | LEU | A | 267 | 12927 | 12661 | 11706 | 2753 | 2933 | 388 | O |
| ATOM | 2073 | N | GLU | A | 268 | 46.001 | 65.276 | 28.983 | 1.00 | 95.14 | | N |
| ANISOU | 2073 | N | GLU | A | 268 | 12652 | 12427 | 11071 | 3508 | 3120 | 483 | N |
| ATOM | 2074 | CA | GLU | A | 268 | 45.857 | 66.041 | 30.228 | 1.00 | 94.33 | | C |
| ANISOU | 2074 | CA | GLU | A | 268 | 12818 | 12113 | 10910 | 3547 | 3271 | 442 | C |
| ATOM | 2075 | CB | GLU | A | 268 | 45.016 | 67.307 | 29.995 | 1.00 | 95.38 | | C |
| ANISOU | 2075 | CB | GLU | A | 268 | 13187 | 12202 | 10850 | 4065 | 3438 | 512 | C |
| ATOM | 2076 | C | GLU | A | 268 | 45.239 | 65.135 | 31.315 | 1.00 | 97.56 | | C |
| ANISOU | 2076 | C | GLU | A | 268 | 12921 | 12789 | 11360 | 3382 | 3230 | 345 | C |
| ATOM | 2077 | O | GLU | A | 268 | 45.595 | 65.255 | 32.488 | 1.00 | 96.45 | | O |
| ANISOU | 2077 | O | GLU | A | 268 | 12951 | 12453 | 11242 | 3193 | 3290 | 284 | O |
| ATOM | 2078 | N | GLN | A | 269 | 44.359 | 64.195 | 30.911 | 1.00 | 94.30 | | N |
| ANISOU | 2078 | N | GLN | A | 269 | 12063 | 12816 | 10951 | 3425 | 3130 | 324 | N |
| ATOM | 2079 | CA | GLN | A | 269 | 43.768 | 63.198 | 31.803 | 1.00 | 94.37 | | C |
| ANISOU | 2079 | CA | GLN | A | 269 | 11768 | 13092 | 10996 | 3227 | 3096 | 230 | C |
| ATOM | 2080 | CB | GLN | A | 269 | 42.526 | 62.544 | 31.146 | 1.00 | 96.17 | | C |
| ANISOU | 2080 | CB | GLN | A | 269 | 11539 | 13839 | 11163 | 3411 | 3040 | 212 | C |
| ATOM | 2081 | CG | GLN | A | 269 | 42.095 | 61.194 | 31.771 | 1.00 | 120.92 | | C |
| ANISOU | 2081 | CG | GLN | A | 269 | 14314 | 17258 | 14372 | 3067 | 2962 | 112 | C |
| ATOM | 2082 | CD | GLN | A | 269 | 41.100 | 60.394 | 30.956 | 1.00 | 145.57 | | C |
| ANISOU | 2082 | CD | GLN | A | 269 | 16977 | 20893 | 17439 | 3176 | 2896 | 77 | C |
| ATOM | 2083 | OE1 | GLN | A | 269 | 40.655 | 60.803 | 29.878 | 1.00 | 142.98 | | O |
| ANISOU | 2083 | OE1 | GLN | A | 269 | 16558 | 20732 | 17036 | 3478 | 2862 | 130 | O |
| ATOM | 2084 | NE2 | GLN | A | 269 | 40.729 | 59.224 | 31.463 | 1.00 | 136.98 | | N |
| ANISOU | 2084 | NE2 | GLN | A | 269 | 15596 | 20074 | 16376 | 2922 | 2880 | −20 | N |
| ATOM | 2085 | C | GLN | A | 269 | 44.831 | 62.133 | 32.145 | 1.00 | 97.75 | | C |
| ANISOU | 2085 | C | GLN | A | 269 | 12157 | 13400 | 11585 | 2731 | 2971 | 180 | C |
| ATOM | 2086 | O | GLN | A | 269 | 45.000 | 61.800 | 33.318 | 1.00 | 97.16 | | O |
| ANISOU | 2086 | O | GLN | A | 269 | 12100 | 13272 | 11545 | 2498 | 2991 | 109 | O |
| ATOM | 2087 | N | ILE | A | 270 | 45.532 | 61.611 | 31.107 | 1.00 | 94.16 | | N |
| ANISOU | 2087 | N | ILE | A | 270 | 11647 | 12920 | 11211 | 2596 | 2844 | 217 | N |
| ATOM | 2088 | CA | ILE | A | 270 | 46.566 | 60.573 | 31.167 | 1.00 | 93.87 | | C |
| ANISOU | 2088 | CA | ILE | A | 270 | 11558 | 12801 | 11309 | 2183 | 2710 | 182 | C |
| ATOM | 2089 | CB | ILE | A | 270 | 47.187 | 60.379 | 29.756 | 1.00 | 97.09 | | C |
| ANISOU | 2089 | CB | ILE | A | 270 | 11921 | 13204 | 11765 | 2176 | 2600 | 246 | C |
| ATOM | 2090 | CG1 | ILE | A | 270 | 46.287 | 59.490 | 28.856 | 1.00 | 97.21 | | C |
| ANISOU | 2090 | CG1 | ILE | A | 270 | 11544 | 13640 | 11753 | 2286 | 2511 | 245 | C |
| ATOM | 2091 | CD1 | ILE | A | 270 | 46.083 | 58.001 | 29.302 | 1.00 | 101.65 | | C |
| ANISOU | 2091 | CD1 | ILE | A | 270 | 11798 | 14442 | 12381 | 1966 | 2405 | 162 | C |
| ATOM | 2092 | CG2 | ILE | A | 270 | 48.649 | 59.893 | 29.766 | 1.00 | 98.62 | | C |
| ANISOU | 2092 | CG2 | ILE | A | 270 | 12211 | 13181 | 12080 | 1812 | 2495 | 230 | C |
| ATOM | 2093 | C | ILE | A | 270 | 47.602 | 60.837 | 32.278 | 1.00 | 98.38 | | C |
| ANISOU | 2093 | C | ILE | A | 270 | 12431 | 13021 | 11928 | 1935 | 2748 | 142 | C |
| ATOM | 2094 | O | ILE | A | 270 | 48.089 | 61.960 | 32.436 | 1.00 | 98.07 | | O |
| ANISOU | 2094 | O | ILE | A | 270 | 12723 | 12688 | 11852 | 2030 | 2847 | 168 | O |
| ATOM | 2095 | N | TYR | A | 271 | 47.880 | 59.768 | 33.053 | 1.00 | 95.16 | | N |
| ANISOU | 2095 | N | TYR | A | 271 | 11909 | 12663 | 11584 | 1625 | 2676 | 72 | N |
| ATOM | 2096 | CA | TYR | A | 271 | 48.743 | 59.612 | 34.236 | 1.00 | 94.88 | | C |
| ANISOU | 2096 | CA | TYR | A | 271 | 12065 | 12400 | 11586 | 1354 | 2675 | 10 | C |
| ATOM | 2097 | CB | TYR | A | 271 | 49.000 | 58.099 | 34.476 | 1.00 | 98.03 | | C |
| ANISOU | 2097 | CB | TYR | A | 271 | 12259 | 12937 | 12052 | 1050 | 2546 | −38 | C |
| ATOM | 2098 | C | TYR | A | 271 | 50.099 | 60.390 | 34.241 | 1.00 | 93.22 | | C |
| ANISOU | 2098 | C | TYR | A | 271 | 12181 | 11833 | 11407 | 1249 | 2680 | 18 | C |
| ATOM | 2099 | O | TYR | A | 271 | 50.316 | 61.200 | 35.150 | 1.00 | 93.00 | | O |
| ANISOU | 2099 | O | TYR | A | 271 | 12413 | 11591 | 11331 | 1257 | 2783 | −15 | O |
| ATOM | 2100 | N | ASP | A | 272 | 50.995 | 60.116 | 33.249 | 1.00 | 84.52 | | N |
| ANISOU | 2100 | N | ASP | A | 272 | 11059 | 10681 | 10375 | 1139 | 2574 | 53 | N |
| ATOM | 2101 | CA | ASP | A | 272 | 52.378 | 60.598 | 33.066 | 1.00 | 81.74 | | C |
| ANISOU | 2101 | CA | ASP | A | 272 | 10940 | 10055 | 10063 | 976 | 2555 | 49 | C |
| ATOM | 2102 | CB | ASP | A | 272 | 52.481 | 62.094 | 32.672 | 1.00 | 83.26 | | C |
| ANISOU | 2102 | CB | ASP | A | 272 | 11433 | 10009 | 10193 | 1173 | 2699 | 96 | C |
| ATOM | 2103 | CG | ASP | A | 272 | 53.889 | 62.577 | 32.295 | 1.00 | 88.41 | | C |
| ANISOU | 2103 | CG | ASP | A | 272 | 12300 | 10408 | 10884 | 976 | 2689 | 88 | C |
| ATOM | 2104 | OD1 | ASP | A | 272 | 54.624 | 61.815 | 31.624 | 1.00 | 88.88 | | O |
| ANISOU | 2104 | OD1 | ASP | A | 272 | 12207 | 10543 | 11021 | 814 | 2559 | 96 | O |
| ATOM | 2105 | OD2 | ASP | A | 272 | 54.245 | 63.718 | 32.663 | 1.00 | 90.37 | | O |
| ANISOU | 2105 | OD2 | ASP | A | 272 | 12871 | 10387 | 11078 | 979 | 2821 | 67 | O |
| ATOM | 2106 | C | ASP | A | 272 | 53.279 | 60.252 | 34.270 | 1.00 | 79.90 | | C |
| ANISOU | 2106 | C | ASP | A | 272 | 10814 | 9693 | 9853 | 684 | 2513 | −43 | C |
| ATOM | 2107 | O | ASP | A | 272 | 53.652 | 61.132 | 35.056 | 1.00 | 79.51 | | O |
| ANISOU | 2107 | O | ASP | A | 272 | 11025 | 9425 | 9760 | 650 | 2606 | −88 | O |
| ATOM | 2108 | N | LEU | A | 273 | 53.626 | 58.959 | 34.403 | 1.00 | 71.86 | | N |
| ANISOU | 2108 | N | LEU | A | 273 | 9605 | 8810 | 8888 | 482 | 2373 | −74 | N |
| ATOM | 2109 | CA | LEU | A | 273 | 54.525 | 58.483 | 35.446 | 1.00 | 69.61 | | C |
| ANISOU | 2109 | CA | LEU | A | 273 | 9398 | 8441 | 8610 | 231 | 2309 | −156 | C |
| ATOM | 2110 | CB | LEU | A | 273 | 54.276 | 57.000 | 35.787 | 1.00 | 69.62 | | C |
| ANISOU | 2110 | CB | LEU | A | 273 | 9191 | 8637 | 8627 | 110 | 2202 | −177 | C |
| ATOM | 2111 | CG | LEU | A | 273 | 54.521 | 56.528 | 37.246 | 1.00 | 74.19 | | C |
| ANISOU | 2111 | CG | LEU | A | 273 | 9852 | 9178 | 9160 | −47 | 2187 | −256 | C |
| ATOM | 2112 | CD1 | LEU | A | 273 | 54.011 | 55.108 | 37.449 | 1.00 | 74.38 | | C |
| ANISOU | 2112 | CD1 | LEU | A | 273 | 9694 | 9387 | 9182 | −128 | 2118 | −257 | C |
| ATOM | 2113 | CD2 | LEU | A | 273 | 55.989 | 56.546 | 37.630 | 1.00 | 76.40 | | C |
| ANISOU | 2113 | CD2 | LEU | A | 273 | 10269 | 9318 | 9443 | −236 | 2101 | −316 | C |
| ATOM | 2114 | C | LEU | A | 273 | 55.936 | 58.666 | 34.909 | 1.00 | 69.02 | | C |
| ANISOU | 2114 | C | LEU | A | 273 | 9409 | 8235 | 8579 | 73 | 2236 | −168 | C |
| ATOM | 2115 | O | LEU | A | 273 | 56.308 | 58.056 | 33.891 | 1.00 | 68.50 | | O |
| ANISOU | 2115 | O | LEU | A | 273 | 9196 | 8262 | 8568 | 42 | 2137 | −126 | O |
| ATOM | 2116 | N | LEU | A | 274 | 56.701 | 59.534 | 35.588 | 1.00 | 62.09 | | N |
| ANISOU | 2116 | N | LEU | A | 274 | 8768 | 7155 | 7670 | −34 | 2291 | −234 | N |
| ATOM | 2117 | CA | LEU | A | 274 | 58.089 | 59.862 | 35.282 | 1.00 | 60.60 | | C |
| ANISOU | 2117 | CA | LEU | A | 274 | 8674 | 6847 | 7506 | −221 | 2245 | −275 | C |
| ATOM | 2118 | CB | LEU | A | 274 | 58.605 | 60.923 | 36.254 | 1.00 | 60.93 | | C |
| ANISOU | 2118 | CB | LEU | A | 274 | 8994 | 6671 | 7485 | −325 | 2345 | −366 | C |
| ATOM | 2119 | CG | LEU | A | 274 | 57.814 | 62.209 | 36.385 | 1.00 | 66.48 | | C |
| ANISOU | 2119 | CG | LEU | A | 274 | 9930 | 7201 | 8129 | −132 | 2536 | −337 | C |
| ATOM | 2120 | CD1 | LEU | A | 274 | 56.924 | 62.184 | 37.647 | 1.00 | 67.06 | | C |
| ANISOU | 2120 | CD1 | LEU | A | 274 | 10049 | 7293 | 8139 | −49 | 2594 | −374 | C |
| ATOM | 2121 | CD2 | LEU | A | 274 | 58.756 | 63.402 | 36.448 | 1.00 | 69.13 | | C |
| ANISOU | 2121 | CD2 | LEU | A | 274 | 10560 | 7277 | 8429 | −263 | 2640 | −395 | C |
| ATOM | 2122 | C | LEU | A | 274 | 58.947 | 58.611 | 35.430 | 1.00 | 61.56 | | C |
| ANISOU | 2122 | C | LEU | A | 274 | 8626 | 7106 | 7657 | −409 | 2069 | −318 | C |
| ATOM | 2123 | O | LEU | A | 274 | 58.854 | 57.916 | 36.434 | 1.00 | 61.57 | | O |
| ANISOU | 2123 | O | LEU | A | 274 | 8595 | 7174 | 7626 | −471 | 2016 | −368 | O |
| ATOM | 2124 | N | ALA | A | 275 | 59.762 | 58.307 | 34.437 | 1.00 | 56.22 | | N |
| ANISOU | 2124 | N | ALA | A | 275 | 7856 | 6475 | 7032 | −480 | 1983 | −295 | N |
| ATOM | 2125 | CA | ALA | A | 275 | 60.617 | 57.134 | 34.525 | 1.00 | 55.04 | | C |
| ANISOU | 2125 | CA | ALA | A | 275 | 7557 | 6461 | 6897 | −622 | 1818 | −331 | C |
| ATOM | 2126 | CB | ALA | A | 275 | 60.103 | 56.018 | 33.634 | 1.00 | 55.92 | | C |
| ANISOU | 2126 | CB | ALA | A | 275 | 7454 | 6739 | 7052 | −531 | 1735 | −248 | C |
| ATOM | 2127 | C | ALA | A | 275 | 62.046 | 57.473 | 34.194 | 1.00 | 55.97 | | C |
| ANISOU | 2127 | C | ALA | A | 275 | 7708 | 6536 | 7023 | −793 | 1772 | −388 | C |
| ATOM | 2128 | O | ALA | A | 275 | 62.316 | 58.449 | 33.506 | 1.00 | 55.15 | | O |
| ANISOU | 2128 | O | ALA | A | 275 | 7701 | 6315 | 6937 | −799 | 1862 | −370 | O |
| ATOM | 2129 | N | VAL | A | 276 | 62.962 | 56.699 | 34.740 | 1.00 | 51.11 | | N |
| ANISOU | 2129 | N | VAL | A | 276 | 7022 | 6017 | 6378 | −931 | 1642 | −464 | N |
| ATOM | 2130 | CA | VAL | A | 276 | 64.386 | 56.878 | 34.558 | 1.00 | 50.04 | | C |
| ANISOU | 2130 | CA | VAL | A | 276 | 6873 | 5906 | 6235 | −1106 | 1579 | −542 | C |
| ATOM | 2131 | CB | VAL | A | 276 | 64.965 | 57.685 | 35.750 | 1.00 | 53.53 | | C |
| ANISOU | 2131 | CB | VAL | A | 276 | 7478 | 6258 | 6604 | −1261 | 1622 | −674 | C |
| ATOM | 2132 | CG1 | VAL | A | 276 | 66.375 | 57.257 | 36.122 | 1.00 | 53.44 | | C |
| ANISOU | 2132 | CG1 | VAL | A | 276 | 7375 | 6390 | 6541 | −1438 | 1486 | −791 | C |
| ATOM | 2133 | CG2 | VAL | A | 276 | 64.920 | 59.176 | 35.460 | 1.00 | 53.28 | | C |
| ANISOU | 2133 | CG2 | VAL | A | 276 | 7652 | 6014 | 6578 | −1306 | 1799 | −685 | C |
| ATOM | 2134 | C | VAL | A | 276 | 64.986 | 55.492 | 34.353 | 1.00 | 53.70 | | C |
| ANISOU | 2134 | C | VAL | A | 276 | 7144 | 6566 | 6694 | −1117 | 1403 | −540 | C |
| ATOM | 2135 | O | VAL | A | 276 | 64.440 | 54.502 | 34.840 | 1.00 | 53.30 | | O |
| ANISOU | 2135 | O | VAL | A | 276 | 7044 | 6591 | 6619 | −1040 | 1339 | −515 | O |
| ATOM | 2136 | N | ARG | A | 277 | 66.048 | 55.417 | 33.560 | 1.00 | 50.61 | | N |
| ANISOU | 2136 | N | ARG | A | 277 | 6658 | 6253 | 6321 | −1204 | 1341 | −560 | N |
| ATOM | 2137 | CA | ARG | A | 277 | 66.729 | 54.177 | 33.245 | 1.00 | 50.70 | | C |
| ANISOU | 2137 | CA | ARG | A | 277 | 6499 | 6450 | 6317 | −1193 | 1180 | −557 | C |
| ATOM | 2138 | CB | ARG | A | 277 | 66.642 | 53.885 | 31.741 | 1.00 | 52.91 | | C |
| ANISOU | 2138 | CB | ARG | A | 277 | 6667 | 6768 | 6671 | −1114 | 1173 | −455 | C |
| ATOM | 2139 | CG | ARG | A | 277 | 65.800 | 52.661 | 31.433 | 1.00 | 72.05 | | C |
| ANISOU | 2139 | CG | ARG | A | 277 | 8999 | 9278 | 9101 | −976 | 1092 | −374 | C |
| ATOM | 2140 | CD | ARG | A | 277 | 65.684 | 52.367 | 29.956 | 1.00 | 88.51 | | C |
| ANISOU | 2140 | CD | ARG | A | 277 | 10978 | 11404 | 11248 | −894 | 1083 | −279 | C |
| ATOM | 2141 | NE | ARG | A | 277 | 64.690 | 53.199 | 29.288 | 1.00 | 102.69 | | N |
| ANISOU | 2141 | NE | ARG | A | 277 | 12823 | 13095 | 13100 | −805 | 1211 | −202 | N |
| ATOM | 2142 | CZ | ARG | A | 277 | 64.282 | 52.996 | 28.044 | 1.00 | 122.00 | | C |
| ANISOU | 2142 | CZ | ARG | A | 277 | 15190 | 15575 | 15590 | −704 | 1215 | −113 | C |
| ATOM | 2143 | NH1 | ARG | A | 277 | 64.772 | 51.985 | 27.332 | 1.00 | 113.48 | | N |
| ANISOU | 2143 | NH1 | ARG | A | 277 | 13986 | 14616 | 14514 | −694 | 1104 | −91 | N |
| ATOM | 2144 | NH2 | ARG | A | 277 | 63.388 | 53.808 | 27.493 | 1.00 | 107.79 | | N |
| ANISOU | 2144 | NH2 | ARG | A | 277 | 13441 | 13696 | 13818 | −594 | 1329 | −47 | N |
| ATOM | 2145 | C | ARG | A | 277 | 68.167 | 54.249 | 33.646 | 1.00 | 54.38 | | C |
| ANISOU | 2145 | C | ARG | A | 277 | 6914 | 7029 | 6719 | −1347 | 1095 | −681 | C |
| ATOM | 2146 | O | ARG | A | 277 | 68.808 | 55.286 | 33.478 | 1.00 | 54.14 | | O |
| ANISOU | 2146 | O | ARG | A | 277 | 6923 | 6952 | 6696 | −1494 | 1166 | −746 | O |
| ATOM | 2147 | N | VAL | A | 278 | 68.681 | 53.138 | 34.161 | 1.00 | 50.80 | | N |
| ANISOU | 2147 | N | VAL | A | 278 | 6377 | 6734 | 6190 | −1312 | 947 | −718 | N |
| ATOM | 2148 | CA | VAL | A | 278 | 70.084 | 52.977 | 34.513 | 1.00 | 50.49 | | C |
| ANISOU | 2148 | CA | VAL | A | 278 | 6242 | 6875 | 6068 | −1413 | 833 | −838 | C |
| ATOM | 2149 | CB | VAL | A | 278 | 70.327 | 52.777 | 36.031 | 1.00 | 52.92 | | C |
| ANISOU | 2149 | CB | VAL | A | 278 | 6618 | 7238 | 6253 | −1438 | 766 | −947 | C |
| ATOM | 2150 | CG1 | VAL | A | 278 | 71.761 | 52.365 | 36.305 | 1.00 | 52.35 | | C |
| ANISOU | 2150 | CG1 | VAL | A | 278 | 6403 | 7416 | 6071 | −1486 | 616 | −1065 | C |
| ATOM | 2151 | CG2 | VAL | A | 278 | 69.992 | 54.042 | 36.810 | 1.00 | 52.54 | | C |
| ANISOU | 2151 | CG2 | VAL | A | 278 | 6731 | 7030 | 6204 | −1569 | 895 | −1020 | C |
| ATOM | 2152 | C | VAL | A | 278 | 70.637 | 51.833 | 33.638 | 1.00 | 56.51 | | C |
| ANISOU | 2152 | C | VAL | A | 278 | 6839 | 7808 | 6824 | −1312 | 707 | −787 | C |
| ATOM | 2153 | O | VAL | A | 278 | 70.208 | 50.685 | 33.779 | 1.00 | 57.25 | | O |
| ANISOU | 2153 | O | VAL | A | 278 | 6937 | 7932 | 6883 | −1166 | 631 | −725 | O |
| ATOM | 2154 | N | ILE | A | 279 | 71.512 | 52.172 | 32.675 | 1.00 | 52.45 | | N |
| ANISOU | 2154 | N | ILE | A | 279 | 6201 | 7385 | 6341 | −1394 | 704 | −809 | N |
| ATOM | 2155 | CA | ILE | A | 279 | 72.151 | 51.182 | 31.805 | 1.00 | 51.64 | | C |
| ANISOU | 2155 | CA | ILE | A | 279 | 5941 | 7456 | 6225 | −1299 | 592 | −772 | C |
| ATOM | 2156 | CB | ILE | A | 279 | 72.004 | 51.451 | 30.278 | 1.00 | 54.25 | | C |
| ANISOU | 2156 | CB | ILE | A | 279 | 6215 | 7733 | 6663 | −1295 | 668 | −676 | C |
| ATOM | 2157 | CG1 | ILE | A | 279 | 70.552 | 51.822 | 29.896 | 1.00 | 54.27 | | C |
| ANISOU | 2157 | CG1 | ILE | A | 279 | 6343 | 7516 | 6759 | −1219 | 785 | −555 | C |
| ATOM | 2158 | CD1 | ILE | A | 279 | 70.283 | 53.271 | 29.486 | 1.00 | 57.54 | | C |
| ANISOU | 2158 | CD1 | ILE | A | 279 | 6859 | 7757 | 7245 | −1319 | 957 | −542 | C |
| ATOM | 2159 | CG2 | ILE | A | 279 | 72.463 | 50.231 | 29.473 | 1.00 | 54.45 | | C |
| ANISOU | 2159 | CG2 | ILE | A | 279 | 6101 | 7924 | 6663 | −1165 | 547 | −629 | C |
| ATOM | 2160 | C | ILE | A | 279 | 73.597 | 51.054 | 32.284 | 1.00 | 55.39 | | C |
| ANISOU | 2160 | C | ILE | A | 279 | 6281 | 8179 | 6585 | −1372 | 476 | −914 | C |
| ATOM | 2161 | O | ILE | A | 279 | 74.329 | 52.040 | 32.329 | 1.00 | 54.47 | | O |
| ANISOU | 2161 | O | ILE | A | 279 | 6120 | 8110 | 6466 | −1566 | 528 | −1020 | O |
| ATOM | 2162 | N | LEU | A | 280 | 73.975 | 49.858 | 32.712 | 1.00 | 52.82 | | N |
| ANISOU | 2162 | N | LEU | A | 280 | 5907 | 8013 | 6149 | −1218 | 327 | −924 | N |
| ATOM | 2163 | CA | LEU | A | 280 | 75.289 | 49.623 | 33.283 | 1.00 | 53.87 | | C |
| ANISOU | 2163 | CA | LEU | A | 280 | 5904 | 8420 | 6143 | −1235 | 199 | −1062 | C |
| ATOM | 2164 | CB | LEU | A | 280 | 75.128 | 49.046 | 34.714 | 1.00 | 53.77 | | C |
| ANISOU | 2164 | CB | LEU | A | 280 | 6002 | 8436 | 5993 | −1132 | 110 | −1110 | C |
| ATOM | 2165 | CG | LEU | A | 280 | 74.235 | 49.770 | 35.704 | 1.00 | 57.61 | | C |
| ANISOU | 2165 | CG | LEU | A | 280 | 6676 | 8710 | 6502 | −1217 | 208 | −1123 | C |
| ATOM | 2166 | CD1 | LEU | A | 280 | 73.377 | 48.798 | 36.473 | 1.00 | 56.97 | | C |
| ANISOU | 2166 | CD1 | LEU | A | 280 | 6757 | 8541 | 6347 | −1036 | 166 | −1053 | C |
| ATOM | 2167 | CD2 | LEU | A | 280 | 75.044 | 50.597 | 36.630 | 1.00 | 60.08 | | C |
| ANISOU | 2167 | CD2 | LEU | A | 280 | 6956 | 9134 | 6736 | −1394 | 198 | −1295 | C |
| ATOM | 2168 | C | LEU | A | 280 | 76.062 | 48.611 | 32.475 | 1.00 | 62.46 | | C |
| ANISOU | 2168 | C | LEU | A | 280 | 6834 | 9720 | 7178 | −1087 | 84 | −1036 | C |
| ATOM | 2169 | O | LEU | A | 280 | 75.551 | 47.510 | 32.267 | 1.00 | 62.55 | | O |
| ANISOU | 2169 | O | LEU | A | 280 | 6916 | 9689 | 7163 | −884 | 27 | −935 | O |
| ATOM | 2170 | N | ASP | A | 281 | 77.306 | 48.919 | 32.066 | 1.00 | 62.03 | | N |
| ANISOU | 2170 | N | ASP | A | 281 | 6573 | 9904 | 7089 | −1183 | 49 | −1136 | N |
| ATOM | 2171 | CA | ASP | A | 281 | 78.075 | 47.860 | 31.423 | 1.00 | 63.25 | | C |
| ANISOU | 2171 | CA | ASP | A | 281 | 6581 | 10285 | 7164 | −998 | −72 | −1117 | C |
| ATOM | 2172 | CB | ASP | A | 281 | 78.576 | 48.118 | 29.974 | 1.00 | 66.62 | | C |
| ANISOU | 2172 | CB | ASP | A | 281 | 6850 | 10783 | 7679 | −1066 | −17 | −1086 | C |
| ATOM | 2173 | CG | ASP | A | 281 | 79.093 | 49.485 | 29.560 | 1.00 | 85.29 | | C |
| ANISOU | 2173 | CG | ASP | A | 281 | 9115 | 13172 | 10118 | −1362 | 108 | −1175 | C |
| ATOM | 2174 | OD1 | ASP | A | 281 | 79.036 | 50.427 | 30.394 | 1.00 | 88.08 | | O |
| ANISOU | 2174 | OD1 | ASP | A | 281 | 9536 | 13457 | 10473 | −1555 | 171 | −1270 | O |
| ATOM | 2175 | OD2 | ASP | A | 281 | 79.557 | 49.616 | 28.392 | 1.00 | 90.32 | | O |
| ANISOU | 2175 | OD2 | ASP | A | 281 | 9628 | 13883 | 10806 | −1408 | 154 | −1151 | O |
| ATOM | 2176 | C | ASP | A | 281 | 79.155 | 47.411 | 32.363 | 1.00 | 66.63 | | C |
| ANISOU | 2176 | C | ASP | A | 281 | 6892 | 11025 | 7398 | −917 | −226 | −1257 | C |
| ATOM | 2177 | O | ASP | A | 281 | 79.959 | 48.231 | 32.795 | 1.00 | 64.91 | | O |
| ANISOU | 2177 | O | ASP | A | 281 | 6539 | 10987 | 7138 | −1108 | −226 | −1411 | O |
| ATOM | 2178 | N | PRO | A | 282 | 79.118 | 46.132 | 32.788 | 1.00 | 66.05 | | N |
| ANISOU | 2178 | N | PRO | A | 282 | 6898 | 11008 | 7190 | −638 | −352 | −1212 | N |
| ATOM | 2179 | CA | PRO | A | 282 | 80.124 | 45.662 | 33.758 | 1.00 | 67.93 | | C |
| ANISOU | 2179 | CA | PRO | A | 282 | 7045 | 11553 | 7211 | −511 | −508 | −1342 | C |
| ATOM | 2180 | CB | PRO | A | 282 | 79.620 | 44.262 | 34.156 | 1.00 | 69.24 | | C |
| ANISOU | 2180 | CB | PRO | A | 282 | 7426 | 11625 | 7258 | −195 | −590 | −1234 | C |
| ATOM | 2181 | CG | PRO | A | 282 | 78.790 | 43.822 | 33.022 | 1.00 | 72.58 | | C |
| ANISOU | 2181 | CG | PRO | A | 282 | 7945 | 11816 | 7816 | −147 | −511 | −1072 | C |
| ATOM | 2182 | CD | PRO | A | 282 | 78.172 | 45.057 | 32.424 | 1.00 | 67.56 | | C |
| ANISOU | 2182 | CD | PRO | A | 282 | 7282 | 10987 | 7399 | −428 | −352 | −1048 | C |
| ATOM | 2183 | C | PRO | A | 282 | 81.557 | 45.615 | 33.233 | 1.00 | 76.95 | | C |
| ANISOU | 2183 | C | PRO | A | 282 | 7883 | 13096 | 8257 | −490 | −600 | −1455 | C |
| ATOM | 2184 | O | PRO | A | 282 | 81.796 | 45.596 | 32.023 | 1.00 | 76.11 | | O |
| ANISOU | 2184 | O | PRO | A | 282 | 7657 | 13027 | 8236 | −511 | −558 | −1407 | O |
| ATOM | 2185 | N | LYS | A | 283 | 82.515 | 45.596 | 34.170 | 1.00 | 78.28 | | N |
| ANISOU | 2185 | N | LYS | A | 283 | 7920 | 13587 | 8237 | −441 | −727 | −1613 | N |
| ATOM | 2186 | CA | LYS | A | 283 | 83.928 | 45.449 | 33.856 | 1.00 | 80.25 | | C |
| ANISOU | 2186 | CA | LYS | A | 283 | 7850 | 14286 | 8354 | −391 | −834 | −1743 | C |
| ATOM | 2187 | CB | LYS | A | 283 | 84.785 | 45.681 | 35.119 | 1.00 | 81.77 | | C |
| ANISOU | 2187 | CB | LYS | A | 283 | 7908 | 14818 | 8343 | −400 | −960 | −1944 | C |
| ATOM | 2188 | CG | LYS | A | 283 | 86.124 | 46.375 | 34.865 | 1.00 | 86.34 | | C |
| ANISOU | 2188 | CG | LYS | A | 283 | 8095 | 15866 | 8844 | −559 | −1007 | −2143 | C |
| ATOM | 2189 | CD | LYS | A | 283 | 86.929 | 46.509 | 36.152 | 1.00 | 91.32 | | C |
| ANISOU | 2189 | CD | LYS | A | 283 | 8585 | 16846 | 9267 | −586 | −1136 | −2357 | C |
| ATOM | 2190 | CE | LYS | A | 283 | 87.728 | 47.795 | 36.242 | 1.00 | 94.87 | | C |
| ANISOU | 2190 | CE | LYS | A | 283 | 8767 | 17526 | 9753 | −1017 | −1067 | −2571 | C |
| ATOM | 2191 | NZ | LYS | A | 283 | 86.892 | 48.979 | 36.601 | 1.00 | 96.01 | | N |
| ANISOU | 2191 | NZ | LYS | A | 283 | 9126 | 17292 | 10063 | −1371 | −902 | −2577 | N |
| ATOM | 2192 | C | LYS | A | 283 | 84.087 | 43.991 | 33.348 | 1.00 | 90.32 | | C |
| ANISOU | 2192 | C | LYS | A | 283 | 9161 | 15640 | 9518 | 1 | −938 | −1630 | C |
| ATOM | 2193 | O | LYS | A | 283 | 83.418 | 43.100 | 33.885 | 1.00 | 90.31 | | O |
| ANISOU | 2193 | O | LYS | A | 283 | 9424 | 15450 | 9441 | 240 | −981 | −1527 | O |
| ATOM | 2194 | N | PRO | A | 284 | 84.895 | 43.718 | 32.292 | 1.00 | 90.72 | | N |
| ANISOU | 2194 | N | PRO | A | 284 | 8980 | 15944 | 9546 | 73 | −967 | −1644 | N |
| ATOM | 2195 | CA | PRO | A | 284 | 85.047 | 42.325 | 31.829 | 1.00 | 91.49 | | C |
| ANISOU | 2195 | CA | PRO | A | 284 | 9150 | 16092 | 9521 | 464 | −1060 | −1536 | C |
| ATOM | 2196 | CB | PRO | A | 284 | 85.958 | 42.448 | 30.605 | 1.00 | 93.26 | | C |
| ANISOU | 2196 | CB | PRO | A | 284 | 9062 | 16616 | 9758 | 436 | −1057 | −1585 | C |
| ATOM | 2197 | CG | PRO | A | 284 | 85.873 | 43.900 | 30.200 | 1.00 | 97.76 | | C |
| ANISOU | 2197 | CG | PRO | A | 284 | 9496 | 17116 | 10531 | −8 | −904 | −1649 | C |
| ATOM | 2198 | CD | PRO | A | 284 | 85.739 | 44.631 | 31.497 | 1.00 | 93.02 | | C |
| ANISOU | 2198 | CD | PRO | A | 284 | 8951 | 16483 | 9909 | −194 | −907 | −1759 | C |
| ATOM | 2199 | C | PRO | A | 284 | 85.629 | 41.383 | 32.891 | 1.00 | 97.79 | | C |
| ANISOU | 2199 | C | PRO | A | 284 | 10010 | 17109 | 10036 | 820 | −1234 | −1585 | C |
| ATOM | 2200 | O | PRO | A | 284 | 86.483 | 41.777 | 33.705 | 1.00 | 97.58 | | O |
| ANISOU | 2200 | O | PRO | A | 284 | 9792 | 17423 | 9861 | 801 | −1331 | −1756 | O |
| ATOM | 2201 | N | ALA | A | 285 | 85.118 | 40.141 | 32.898 | 1.00 | 95.62 | | N |
| ANISOU | 2201 | N | ALA | A | 285 | 10021 | 16635 | 9677 | 1143 | −1268 | −1438 | N |
| ATOM | 2202 | CA | ALA | A | 285 | 85.504 | 39.102 | 33.847 | 1.00 | 95.97 | | C |
| ANISOU | 2202 | CA | ALA | A | 285 | 10210 | 16817 | 9436 | 1528 | −1414 | −1451 | C |
| ATOM | 2203 | CB | ALA | A | 285 | 84.288 | 38.643 | 34.640 | 1.00 | 96.75 | | C |
| ANISOU | 2203 | CB | ALA | A | 285 | 10723 | 16499 | 9540 | 1573 | −1363 | −1335 | C |
| ATOM | 2204 | C | ALA | A | 285 | 86.213 | 37.893 | 33.193 | 1.00 | 100.23 | | C |
| ANISOU | 2204 | C | ALA | A | 285 | 10743 | 17544 | 9797 | 1930 | −1505 | −1398 | C |
| ATOM | 2205 | O | ALA | A | 285 | 85.866 | 37.503 | 32.061 | 1.00 | 99.72 | | O |
| ANISOU | 2205 | O | ALA | A | 285 | 10738 | 17301 | 9849 | 1942 | −1431 | −1281 | O |
| ATOM | 2206 | N | PRO | A | 286 | 87.198 | 37.292 | 33.928 | 1.00 | 96.21 | | N |
| ANISOU | 2206 | N | PRO | A | 286 | 10163 | 17406 | 8986 | 2274 | −1671 | −1492 | N |
| ATOM | 2207 | CA | PRO | A | 286 | 87.935 | 36.125 | 33.402 | 1.00 | 95.88 | | C |
| ANISOU | 2207 | CA | PRO | A | 286 | 10100 | 17596 | 8733 | 2701 | −1767 | −1460 | C |
| ATOM | 2208 | CB | PRO | A | 286 | 88.877 | 35.753 | 34.557 | 1.00 | 97.33 | | C |
| ANISOU | 2208 | CB | PRO | A | 286 | 10158 | 18234 | 8589 | 3013 | −1951 | −1604 | C |
| ATOM | 2209 | CG | PRO | A | 286 | 88.285 | 36.404 | 35.769 | 1.00 | 101.72 | | C |
| ANISOU | 2209 | CG | PRO | A | 286 | 10826 | 18657 | 9167 | 2811 | −1943 | −1658 | C |
| ATOM | 2210 | CD | PRO | A | 286 | 87.708 | 37.673 | 35.262 | 1.00 | 97.24 | | C |
| ANISOU | 2210 | CD | PRO | A | 286 | 10198 | 17805 | 8943 | 2291 | −1780 | −1646 | C |
| ATOM | 2211 | C | PRO | A | 286 | 87.060 | 34.947 | 32.942 | 1.00 | 99.71 | | C |
| ANISOU | 2211 | C | PRO | A | 286 | 11026 | 17677 | 9181 | 2965 | −1718 | −1262 | C |
| ATOM | 2212 | O | PRO | A | 286 | 87.058 | 34.653 | 31.746 | 1.00 | 99.92 | | O |
| ANISOU | 2212 | O | PRO | A | 286 | 11037 | 17685 | 9244 | 3066 | −1688 | −1190 | O |
| ATOM | 2213 | N | THR | A | 287 | 86.307 | 34.297 | 33.847 | 1.00 | 95.38 | | N |
| ANISOU | 2213 | N | THR | A | 287 | 10878 | 16797 | 8565 | 3050 | −1697 | −1179 | N |
| ATOM | 2214 | CA | THR | A | 287 | 85.438 | 33.182 | 33.460 | 1.00 | 95.06 | | C |
| ANISOU | 2214 | CA | THR | A | 287 | 11289 | 16345 | 8484 | 3247 | −1628 | −1004 | C |
| ATOM | 2215 | CB | THR | A | 287 | 85.319 | 32.137 | 34.578 | 1.00 | 103.74 | | C |
| ANISOU | 2215 | CB | THR | A | 287 | 12764 | 17368 | 9285 | 3603 | −1697 | −973 | C |
| ATOM | 2216 | OG1 | THR | A | 287 | 85.225 | 32.791 | 35.843 | 1.00 | 103.49 | | O |
| ANISOU | 2216 | OG1 | THR | A | 287 | 12673 | 17402 | 9247 | 3441 | −1725 | −1067 | O |
| ATOM | 2217 | CG2 | THR | A | 287 | 86.491 | 31.183 | 34.601 | 1.00 | 103.65 | | C |
| ANISOU | 2217 | CG2 | THR | A | 287 | 12723 | 17732 | 8929 | 4120 | −1852 | −1015 | C |
| ATOM | 2218 | C | THR | A | 287 | 84.081 | 33.681 | 32.987 | 1.00 | 98.86 | | C |
| ANISOU | 2218 | C | THR | A | 287 | 11914 | 16369 | 9278 | 2853 | −1455 | −902 | C |
| ATOM | 2219 | O | THR | A | 287 | 83.553 | 34.626 | 33.573 | 1.00 | 98.67 | | O |
| ANISOU | 2219 | O | THR | A | 287 | 11825 | 16254 | 9412 | 2534 | −1399 | −945 | O |
| ATOM | 2220 | N | ARG | A | 288 | 83.515 | 33.043 | 31.930 | 1.00 | 94.93 | | N |
| ANISOU | 2220 | N | ARG | A | 288 | 11599 | 15608 | 8862 | 2881 | −1372 | −775 | N |
| ATOM | 2221 | CA | ARG | A | 288 | 82.194 | 33.358 | 31.355 | 1.00 | 94.38 | | C |
| ANISOU | 2221 | CA | ARG | A | 288 | 11666 | 15130 | 9063 | 2554 | −1215 | −673 | C |
| ATOM | 2222 | CB | ARG | A | 288 | 81.813 | 32.356 | 30.253 | 1.00 | 95.31 | | C |
| ANISOU | 2222 | CB | ARG | A | 288 | 12009 | 15032 | 9174 | 2692 | −1162 | −552 | C |
| ATOM | 2223 | C | ARG | A | 288 | 81.096 | 33.372 | 32.420 | 1.00 | 97.16 | | C |
| ANISOU | 2223 | C | ARG | A | 288 | 12314 | 15161 | 9439 | 2420 | −1141 | −633 | C |
| ATOM | 2224 | O | ARG | A | 288 | 80.170 | 34.175 | 32.324 | 1.00 | 96.24 | | O |
| ANISOU | 2224 | O | ARG | A | 288 | 12163 | 14841 | 9564 | 2079 | −1030 | −612 | O |
| ATOM | 2225 | N | GLU | A | 289 | 81.215 | 32.487 | 33.438 | 1.00 | 93.37 | | N |
| ANISOU | 2225 | N | GLU | A | 289 | 12133 | 14649 | 8696 | 2708 | −1197 | −622 | N |
| ATOM | 2226 | CA | GLU | A | 289 | 80.273 | 32.363 | 34.551 | 1.00 | 92.59 | | C |
| ANISOU | 2226 | CA | GLU | A | 289 | 12349 | 14267 | 8564 | 2632 | −1130 | −586 | C |
| ATOM | 2227 | CB | GLU | A | 289 | 80.372 | 30.997 | 35.257 | 1.00 | 94.12 | | C |
| ANISOU | 2227 | CB | GLU | A | 289 | 12980 | 14344 | 8436 | 3016 | −1162 | −527 | C |
| ATOM | 2228 | CG | GLU | A | 289 | 81.776 | 30.490 | 35.535 | 1.00 | 106.85 | | C |
| ANISOU | 2228 | CG | GLU | A | 289 | 14518 | 16334 | 9746 | 3450 | −1332 | −593 | C |
| ATOM | 2229 | CD | GLU | A | 289 | 81.799 | 29.029 | 35.939 | 1.00 | 134.19 | | C |
| ANISOU | 2229 | CD | GLU | A | 289 | 18477 | 19619 | 12890 | 3858 | −1338 | −506 | C |
| ATOM | 2230 | OE1 | GLU | A | 289 | 81.700 | 28.162 | 35.039 | 1.00 | 130.84 | | O |
| ANISOU | 2230 | OE1 | GLU | A | 289 | 18255 | 19030 | 12429 | 3989 | −1290 | −418 | O |
| ATOM | 2231 | OE2 | GLU | A | 289 | 81.902 | 28.750 | 37.156 | 1.00 | 128.78 | | O |
| ANISOU | 2231 | OE2 | GLU | A | 289 | 18001 | 18947 | 11981 | 4046 | −1383 | −524 | O |
| ATOM | 2232 | C | GLU | A | 289 | 80.377 | 33.519 | 35.530 | 1.00 | 94.09 | | C |
| ANISOU | 2232 | C | GLU | A | 289 | 12325 | 14612 | 8813 | 2435 | −1159 | −698 | C |
| ATOM | 2233 | O | GLU | A | 289 | 79.349 | 34.123 | 35.838 | 1.00 | 93.13 | | O |
| ANISOU | 2233 | O | GLU | A | 289 | 12263 | 14249 | 8874 | 2137 | −1046 | −675 | O |
| ATOM | 2234 | N | SER | A | 290 | 81.611 | 33.873 | 35.967 | 1.00 | 89.28 | | N |
| ANISOU | 2234 | N | SER | A | 290 | 11445 | 14421 | 8055 | 2583 | −1305 | −827 | N |
| ATOM | 2235 | CA | SER | A | 290 | 81.844 | 34.998 | 36.882 | 1.00 | 88.41 | | C |
| ANISOU | 2235 | CA | SER | A | 290 | 11115 | 14493 | 7983 | 2383 | −1342 | −958 | C |
| ATOM | 2236 | CB | SER | A | 290 | 83.263 | 34.982 | 37.441 | 1.00 | 92.46 | | C |
| ANISOU | 2236 | CB | SER | A | 290 | 11398 | 15491 | 8241 | 2643 | −1524 | −1101 | C |
| ATOM | 2237 | OG | SER | A | 290 | 84.205 | 35.615 | 36.588 | 1.00 | 102.95 | | O |
| ANISOU | 2237 | OG | SER | A | 290 | 12275 | 17177 | 9665 | 2513 | −1573 | −1212 | O |
| ATOM | 2238 | C | SER | A | 290 | 81.511 | 36.351 | 36.233 | 1.00 | 90.13 | | C |
| ANISOU | 2238 | C | SER | A | 290 | 11034 | 14690 | 8523 | 1949 | −1248 | −999 | C |
| ATOM | 2239 | O | SER | A | 290 | 81.506 | 37.374 | 36.923 | 1.00 | 90.45 | | O |
| ANISOU | 2239 | O | SER | A | 290 | 10929 | 14810 | 8628 | 1726 | −1243 | −1100 | O |
| ATOM | 2240 | N | GLN | A | 291 | 81.219 | 36.344 | 34.915 | 1.00 | 83.95 | | N |
| ANISOU | 2240 | N | GLN | A | 291 | 10186 | 13788 | 7923 | 1843 | −1168 | −920 | N |
| ATOM | 2241 | CA | GLN | A | 291 | 80.785 | 37.507 | 34.144 | 1.00 | 82.80 | | C |
| ANISOU | 2241 | CA | GLN | A | 291 | 9821 | 13565 | 8072 | 1469 | −1059 | −927 | C |
| ATOM | 2242 | CB | GLN | A | 291 | 81.273 | 37.411 | 32.680 | 1.00 | 83.86 | | C |
| ANISOU | 2242 | CB | GLN | A | 291 | 9759 | 13816 | 8290 | 1489 | −1056 | −898 | C |
| ATOM | 2243 | CG | GLN | A | 291 | 80.814 | 38.550 | 31.756 | 1.00 | 95.13 | | C |
| ANISOU | 2243 | CG | GLN | A | 291 | 11005 | 15133 | 10006 | 1136 | −931 | −884 | C |
| ATOM | 2244 | CD | GLN | A | 291 | 81.293 | 39.908 | 32.197 | 1.00 | 111.43 | | C |
| ANISOU | 2244 | CD | GLN | A | 291 | 12808 | 17379 | 12151 | 867 | −919 | −1021 | C |
| ATOM | 2245 | OE1 | GLN | A | 291 | 82.467 | 40.244 | 32.050 | 1.00 | 105.32 | | O |
| ANISOU | 2245 | OE1 | GLN | A | 291 | 11742 | 16956 | 11318 | 859 | −987 | −1137 | O |
| ATOM | 2246 | NE2 | GLN | A | 291 | 80.393 | 40.719 | 32.749 | 1.00 | 103.89 | | N |
| ANISOU | 2246 | NE2 | GLN | A | 291 | 11957 | 16193 | 11322 | 633 | −824 | −1018 | N |
| ATOM | 2247 | C | GLN | A | 291 | 79.249 | 37.604 | 34.229 | 1.00 | 85.04 | | C |
| ANISOU | 2247 | C | GLN | A | 291 | 10363 | 13419 | 8529 | 1268 | −909 | −818 | C |
| ATOM | 2248 | O | GLN | A | 291 | 78.711 | 38.709 | 34.381 | 1.00 | 84.59 | | O |
| ANISOU | 2248 | O | GLN | A | 291 | 10216 | 13271 | 8653 | 975 | −823 | −848 | O |
| ATOM | 2249 | N | ALA | A | 292 | 78.557 | 36.431 | 34.154 | 1.00 | 79.88 | | N |
| ANISOU | 2249 | N | ALA | A | 292 | 10040 | 12510 | 7800 | 1430 | −872 | −699 | N |
| ATOM | 2250 | CA | ALA | A | 292 | 77.091 | 36.290 | 34.241 | 1.00 | 78.52 | | C |
| ANISOU | 2250 | CA | ALA | A | 292 | 10124 | 11957 | 7751 | 1268 | −731 | −599 | C |
| ATOM | 2251 | CB | ALA | A | 292 | 76.671 | 34.880 | 33.865 | 1.00 | 79.43 | | C |
| ANISOU | 2251 | CB | ALA | A | 292 | 10555 | 11869 | 7755 | 1459 | −706 | −489 | C |
| ATOM | 2252 | C | ALA | A | 292 | 76.606 | 36.611 | 35.642 | 1.00 | 77.88 | | C |
| ANISOU | 2252 | C | ALA | A | 292 | 10192 | 11784 | 7613 | 1204 | −706 | −635 | C |
| ATOM | 2253 | O | ALA | A | 292 | 75.479 | 37.084 | 35.809 | 1.00 | 77.29 | | O |
| ANISOU | 2253 | O | ALA | A | 292 | 10197 | 11478 | 7693 | 979 | −582 | −597 | O |
| ATOM | 2254 | N | LEU | A | 293 | 77.464 | 36.327 | 36.645 | 1.00 | 70.67 | | N |
| ANISOU | 2254 | N | LEU | A | 293 | 9319 | 11070 | 6462 | 1424 | −825 | −708 | N |
| ATOM | 2255 | CA | LEU | A | 293 | 77.234 | 36.619 | 38.048 | 1.00 | 68.63 | | C |
| ANISOU | 2255 | CA | LEU | A | 293 | 9187 | 10780 | 6109 | 1402 | −827 | −760 | C |
| ATOM | 2256 | CB | LEU | A | 293 | 78.190 | 35.810 | 38.935 | 1.00 | 68.46 | | C |
| ANISOU | 2256 | CB | LEU | A | 293 | 9282 | 10967 | 5763 | 1755 | −972 | −809 | C |
| ATOM | 2257 | CG | LEU | A | 293 | 77.682 | 34.465 | 39.466 | 1.00 | 72.77 | | C |
| ANISOU | 2257 | CG | LEU | A | 293 | 10283 | 11266 | 6102 | 1995 | −937 | −704 | C |
| ATOM | 2258 | CD1 | LEU | A | 293 | 77.715 | 33.373 | 38.388 | 1.00 | 73.45 | | C |
| ANISOU | 2258 | CD1 | LEU | A | 293 | 10511 | 11249 | 6146 | 2169 | −922 | −603 | C |
| ATOM | 2259 | CD2 | LEU | A | 293 | 78.524 | 34.003 | 40.623 | 1.00 | 73.65 | | C |
| ANISOU | 2259 | CD2 | LEU | A | 293 | 10511 | 11577 | 5894 | 2310 | −1069 | −768 | C |
| ATOM | 2260 | C | LEU | A | 293 | 77.423 | 38.116 | 38.267 | 1.00 | 70.48 | | C |
| ANISOU | 2260 | C | LEU | A | 293 | 9122 | 11155 | 6502 | 1126 | −819 | −873 | C |
| ATOM | 2261 | O | LEU | A | 293 | 76.612 | 38.703 | 38.977 | 1.00 | 71.01 | | O |
| ANISOU | 2261 | O | LEU | A | 293 | 9280 | 11048 | 6654 | 940 | −728 | −877 | O |
| ATOM | 2262 | N | ARG | A | 294 | 78.450 | 38.748 | 37.623 | 1.00 | 64.89 | | N |
| ANISOU | 2262 | N | ARG | A | 294 | 8071 | 10750 | 5836 | 1084 | −896 | −966 | N |
| ATOM | 2263 | CA | ARG | A | 294 | 78.723 | 40.197 | 37.706 | 1.00 | 63.97 | | C |
| ANISOU | 2263 | CA | ARG | A | 294 | 7680 | 10761 | 5866 | 792 | −871 | −1082 | C |
| ATOM | 2264 | CB | ARG | A | 294 | 80.039 | 40.573 | 36.992 | 1.00 | 64.52 | | C |
| ANISOU | 2264 | CB | ARG | A | 294 | 7394 | 11195 | 5927 | 785 | −960 | −1185 | C |
| ATOM | 2265 | CG | ARG | A | 294 | 80.491 | 42.024 | 37.247 | 1.00 | 74.19 | | C |
| ANISOU | 2265 | CG | ARG | A | 294 | 8362 | 12583 | 7244 | 481 | −940 | −1337 | C |
| ATOM | 2266 | CD | ARG | A | 294 | 81.894 | 42.330 | 36.753 | 1.00 | 75.69 | | C |
| ANISOU | 2266 | CD | ARG | A | 294 | 8195 | 13195 | 7369 | 474 | −1037 | −1472 | C |
| ATOM | 2267 | NE | ARG | A | 294 | 82.915 | 41.656 | 37.557 | 1.00 | 78.98 | | N |
| ANISOU | 2267 | NE | ARG | A | 294 | 8546 | 13965 | 7496 | 757 | −1217 | −1575 | N |
| ATOM | 2268 | CZ | ARG | A | 294 | 84.228 | 41.743 | 37.353 | 1.00 | 95.05 | | C |
| ANISOU | 2268 | CZ | ARG | A | 294 | 10259 | 16447 | 9410 | 812 | −1331 | −1715 | C |
| ATOM | 2269 | NH1 | ARG | A | 294 | 84.707 | 42.485 | 36.361 | 1.00 | 82.83 | | N |
| ANISOU | 2269 | NH1 | ARG | A | 294 | 8430 | 15033 | 8008 | 576 | −1273 | −1770 | N |
| ATOM | 2270 | NH2 | ARG | A | 294 | 85.072 | 41.084 | 38.136 | 1.00 | 84.35 | | N |
| ANISOU | 2270 | NH2 | ARG | A | 294 | 8857 | 15420 | 7770 | 1110 | −1500 | −1803 | N |
| ATOM | 2271 | C | ARG | A | 294 | 77.559 | 40.985 | 37.107 | 1.00 | 65.86 | | C |
| ANISOU | 2271 | C | ARG | A | 294 | 7950 | 10693 | 6379 | 503 | −699 | −1004 | C |
| ATOM | 2272 | O | ARG | A | 294 | 77.206 | 42.044 | 37.622 | 1.00 | 65.26 | | O |
| ANISOU | 2272 | O | ARG | A | 294 | 7868 | 10534 | 6395 | 289 | −627 | −1055 | O |
| ATOM | 2273 | N | GLU | A | 295 | 76.957 | 40.443 | 36.036 | 1.00 | 61.09 | | N |
| ANISOU | 2273 | N | GLU | A | 295 | 7402 | 9923 | 5887 | 522 | −633 | −881 | N |
| ATOM | 2274 | CA | GLU | A | 295 | 75.809 | 40.996 | 35.312 | 1.00 | 60.07 | | C |
| ANISOU | 2274 | CA | GLU | A | 295 | 7302 | 9527 | 5996 | 305 | −481 | −794 | C |
| ATOM | 2275 | CB | GLU | A | 295 | 75.413 | 40.027 | 34.183 | 1.00 | 61.61 | | C |
| ANISOU | 2275 | CB | GLU | A | 295 | 7577 | 9603 | 6230 | 409 | −456 | −671 | C |
| ATOM | 2276 | CG | GLU | A | 295 | 74.639 | 40.669 | 33.043 | 1.00 | 73.33 | | C |
| ANISOU | 2276 | CG | GLU | A | 295 | 8966 | 10943 | 7951 | 211 | −338 | −606 | C |
| ATOM | 2277 | CD | GLU | A | 295 | 75.463 | 41.413 | 32.010 | 1.00 | 93.51 | | C |
| ANISOU | 2277 | CD | GLU | A | 295 | 11246 | 13681 | 10604 | 131 | −352 | −646 | C |
| ATOM | 2278 | OE1 | GLU | A | 295 | 76.695 | 41.552 | 32.193 | 1.00 | 86.45 | | O |
| ANISOU | 2278 | OE1 | GLU | A | 295 | 10188 | 13056 | 9603 | 194 | −452 | −743 | O |
| ATOM | 2279 | OE2 | GLU | A | 295 | 74.867 | 41.851 | 31.001 | 1.00 | 87.91 | | O |
| ANISOU | 2279 | OE2 | GLU | A | 295 | 10480 | 12855 | 10066 | 6 | −259 | −583 | O |
| ATOM | 2280 | C | GLU | A | 295 | 74.613 | 41.247 | 36.241 | 1.00 | 60.81 | | C |
| ANISOU | 2280 | C | GLU | A | 295 | 7605 | 9370 | 6129 | 195 | −377 | −765 | C |
| ATOM | 2281 | O | GLU | A | 295 | 73.968 | 42.289 | 36.146 | 1.00 | 60.18 | | O |
| ANISOU | 2281 | O | GLU | A | 295 | 7470 | 9175 | 6222 | −21 | −269 | −767 | O |
| ATOM | 2282 | N | LYS | A | 296 | 74.342 | 40.295 | 37.147 | 1.00 | 55.12 | | N |
| ANISOU | 2282 | N | LYS | A | 296 | 7135 | 8572 | 5236 | 357 | −404 | −740 | N |
| ATOM | 2283 | CA | LYS | A | 296 | 73.247 | 40.374 | 38.095 | 1.00 | 54.09 | | C |
| ANISOU | 2283 | CA | LYS | A | 296 | 7221 | 8219 | 5112 | 275 | −303 | −712 | C |
| ATOM | 2284 | CB | LYS | A | 296 | 72.711 | 38.982 | 38.417 | 1.00 | 54.64 | | C |
| ANISOU | 2284 | CB | LYS | A | 296 | 7601 | 8125 | 5033 | 445 | −286 | −623 | C |
| ATOM | 2285 | CG | LYS | A | 296 | 72.043 | 38.319 | 37.228 | 1.00 | 57.45 | | C |
| ANISOU | 2285 | CG | LYS | A | 296 | 8005 | 8333 | 5490 | 427 | −216 | −516 | C |
| ATOM | 2286 | CD | LYS | A | 296 | 72.217 | 36.806 | 37.252 | 1.00 | 60.86 | | C |
| ANISOU | 2286 | CD | LYS | A | 296 | 8704 | 8701 | 5719 | 658 | −255 | −456 | C |
| ATOM | 2287 | CE | LYS | A | 296 | 70.896 | 36.079 | 37.226 | 1.00 | 59.04 | | C |
| ANISOU | 2287 | CE | LYS | A | 296 | 8723 | 8188 | 5520 | 563 | −112 | −367 | C |
| ATOM | 2288 | NZ | LYS | A | 296 | 71.086 | 34.608 | 37.290 | 1.00 | 62.77 | | N |
| ANISOU | 2288 | NZ | LYS | A | 296 | 9517 | 8558 | 5775 | 771 | −127 | −312 | N |
| ATOM | 2289 | C | LYS | A | 296 | 73.624 | 41.143 | 39.362 | 1.00 | 61.04 | | C |
| ANISOU | 2289 | C | LYS | A | 296 | 8084 | 9197 | 5912 | 224 | −337 | −827 | C |
| ATOM | 2290 | O | LYS | A | 296 | 72.880 | 42.043 | 39.741 | 1.00 | 61.74 | | O |
| ANISOU | 2290 | O | LYS | A | 296 | 8181 | 9159 | 6120 | 34 | −231 | −842 | O |
| ATOM | 2291 | N | GLN | A | 297 | 74.779 | 40.844 | 39.996 | 1.00 | 58.55 | | N |
| ANISOU | 2291 | N | GLN | A | 297 | 7737 | 9122 | 5386 | 400 | −484 | −915 | N |
| ATOM | 2292 | CA | GLN | A | 297 | 75.202 | 41.535 | 41.226 | 1.00 | 59.10 | | C |
| ANISOU | 2292 | CA | GLN | A | 297 | 7783 | 9320 | 5352 | 359 | −534 | −1042 | C |
| ATOM | 2293 | CB | GLN | A | 297 | 76.554 | 40.986 | 41.776 | 1.00 | 60.96 | | C |
| ANISOU | 2293 | CB | GLN | A | 297 | 7955 | 9883 | 5326 | 609 | −723 | −1138 | C |
| ATOM | 2294 | CG | GLN | A | 297 | 77.861 | 41.734 | 41.403 | 1.00 | 76.71 | | C |
| ANISOU | 2294 | CG | GLN | A | 297 | 9586 | 12237 | 7322 | 537 | −831 | −1284 | C |
| ATOM | 2295 | CD | GLN | A | 297 | 78.118 | 43.036 | 42.153 | 1.00 | 88.84 | | C |
| ANISOU | 2295 | CD | GLN | A | 297 | 10990 | 13867 | 8898 | 287 | −818 | −1434 | C |
| ATOM | 2296 | OE1 | GLN | A | 297 | 77.791 | 43.221 | 43.340 | 1.00 | 77.75 | | O |
| ANISOU | 2296 | OE1 | GLN | A | 297 | 9745 | 12388 | 7407 | 273 | −806 | −1474 | O |
| ATOM | 2297 | NE2 | GLN | A | 297 | 78.717 | 43.978 | 41.454 | 1.00 | 85.89 | | N |
| ANISOU | 2297 | NE2 | GLN | A | 297 | 10335 | 13647 | 8651 | 74 | −807 | −1522 | N |
| ATOM | 2298 | C | GLN | A | 297 | 75.208 | 43.085 | 41.090 | 1.00 | 62.02 | | C |
| ANISOU | 2298 | C | GLN | A | 297 | 7941 | 9716 | 5907 | 61 | −468 | −1132 | C |
| ATOM | 2299 | O | GLN | A | 297 | 74.899 | 43.779 | 42.055 | 1.00 | 63.04 | | O |
| ANISOU | 2299 | O | GLN | A | 297 | 8141 | 9787 | 6026 | −53 | −425 | −1196 | O |
| ATOM | 2300 | N | VAL | A | 298 | 75.561 | 43.594 | 39.907 | 1.00 | 56.40 | | N |
| ANISOU | 2300 | N | VAL | A | 298 | 7003 | 9076 | 5349 | −56 | −451 | −1135 | N |
| ATOM | 2301 | CA | VAL | A | 298 | 75.630 | 45.014 | 39.564 | 1.00 | 55.12 | | C |
| ANISOU | 2301 | CA | VAL | A | 298 | 6668 | 8918 | 5355 | −333 | −370 | −1209 | C |
| ATOM | 2302 | CB | VAL | A | 298 | 76.350 | 45.178 | 38.177 | 1.00 | 58.78 | | C |
| ANISOU | 2302 | CB | VAL | A | 298 | 6888 | 9524 | 5921 | −383 | −385 | −1207 | C |
| ATOM | 2303 | CG1 | VAL | A | 298 | 75.759 | 46.298 | 37.326 | 1.00 | 58.44 | | C |
| ANISOU | 2303 | CG1 | VAL | A | 298 | 6768 | 9343 | 6095 | −653 | −239 | −1206 | C |
| ATOM | 2304 | CG2 | VAL | A | 298 | 77.859 | 45.362 | 38.351 | 1.00 | 58.48 | | C |
| ANISOU | 2304 | CG2 | VAL | A | 298 | 6623 | 9870 | 5727 | −323 | −538 | −1349 | C |
| ATOM | 2305 | C | VAL | A | 298 | 74.226 | 45.640 | 39.620 | 1.00 | 55.81 | | C |
| ANISOU | 2305 | C | VAL | A | 298 | 6909 | 8683 | 5612 | −488 | −195 | −1130 | C |
| ATOM | 2306 | O | VAL | A | 298 | 74.059 | 46.737 | 40.147 | 1.00 | 54.43 | | O |
| ANISOU | 2306 | O | VAL | A | 298 | 6739 | 8455 | 5487 | −667 | −125 | −1207 | O |
| ATOM | 2307 | N | CYS | A | 299 | 73.230 | 44.915 | 39.104 | 1.00 | 51.61 | | N |
| ANISOU | 2307 | N | CYS | A | 299 | 6505 | 7953 | 5152 | −413 | −125 | −986 | N |
| ATOM | 2308 | CA | CYS | A | 299 | 71.847 | 45.348 | 39.029 | 1.00 | 51.24 | | C |
| ANISOU | 2308 | CA | CYS | A | 299 | 6571 | 7643 | 5256 | −525 | 36 | −904 | C |
| ATOM | 2309 | CB | CYS | A | 299 | 71.062 | 44.413 | 38.131 | 1.00 | 51.97 | | C |
| ANISOU | 2309 | CB | CYS | A | 299 | 6724 | 7611 | 5412 | −442 | 79 | −766 | C |
| ATOM | 2310 | SG | CYS | A | 299 | 71.262 | 44.774 | 36.379 | 1.00 | 56.08 | | S |
| ANISOU | 2310 | SG | CYS | A | 299 | 7030 | 8173 | 6105 | −501 | 98 | −715 | S |
| ATOM | 2311 | C | CYS | A | 299 | 71.166 | 45.527 | 40.380 | 1.00 | 56.21 | | C |
| ANISOU | 2311 | C | CYS | A | 299 | 7392 | 8150 | 5816 | −547 | 94 | −930 | C |
| ATOM | 2312 | O | CYS | A | 299 | 70.490 | 46.535 | 40.583 | 1.00 | 56.11 | | O |
| ANISOU | 2312 | O | CYS | A | 299 | 7400 | 8010 | 5911 | −692 | 212 | −943 | O |
| ATOM | 2313 | N | TYR | A | 300 | 71.323 | 44.558 | 41.300 | 1.00 | 52.58 | | N |
| ANISOU | 2313 | N | TYR | A | 300 | 7091 | 7720 | 5168 | −389 | 21 | −933 | N |
| ATOM | 2314 | CA | TYR | A | 300 | 70.709 | 44.642 | 42.625 | 1.00 | 52.04 | | C |
| ANISOU | 2314 | CA | TYR | A | 300 | 7223 | 7542 | 5009 | −392 | 74 | −955 | C |
| ATOM | 2315 | CB | TYR | A | 300 | 70.615 | 43.268 | 43.299 | 1.00 | 52.18 | | C |
| ANISOU | 2315 | CB | TYR | A | 300 | 7465 | 7533 | 4828 | −189 | 25 | −903 | C |
| ATOM | 2316 | CG | TYR | A | 300 | 69.783 | 42.262 | 42.530 | 1.00 | 52.25 | | C |
| ANISOU | 2316 | CG | TYR | A | 300 | 7578 | 7387 | 4887 | −140 | 96 | −768 | C |
| ATOM | 2317 | CD2 | TYR | A | 300 | 68.464 | 41.996 | 42.889 | 1.00 | 52.49 | | C |
| ANISOU | 2317 | CD2 | TYR | A | 300 | 7790 | 7204 | 4948 | −200 | 243 | −696 | C |
| ATOM | 2318 | CE2 | TYR | A | 300 | 67.705 | 41.052 | 42.197 | 1.00 | 52.84 | | C |
| ANISOU | 2318 | CE2 | TYR | A | 300 | 7926 | 7124 | 5027 | −188 | 312 | −591 | C |
| ATOM | 2319 | CZ | TYR | A | 300 | 68.272 | 40.354 | 41.143 | 1.00 | 57.06 | | C |
| ANISOU | 2319 | CZ | TYR | A | 300 | 8394 | 7726 | 5561 | −97 | 231 | −552 | C |
| ATOM | 2320 | OH | TYR | A | 300 | 67.574 | 39.406 | 40.441 | 1.00 | 57.38 | | O |
| ANISOU | 2320 | OH | TYR | A | 300 | 8539 | 7643 | 5620 | −98 | 297 | −463 | O |
| ATOM | 2321 | CE1 | TYR | A | 300 | 69.586 | 40.584 | 40.791 | 1.00 | 52.94 | | C |
| ANISOU | 2321 | CE1 | TYR | A | 300 | 7700 | 7410 | 5004 | −8 | 85 | −613 | C |
| ATOM | 2322 | CD1 | TYR | A | 300 | 70.335 | 41.528 | 41.486 | 1.00 | 53.47 | | C |
| ANISOU | 2322 | CD1 | TYR | A | 300 | 7655 | 7622 | 5040 | −38 | 19 | −724 | C |
| ATOM | 2323 | C | TYR | A | 300 | 71.461 | 45.636 | 43.483 | 1.00 | 58.28 | | C |
| ANISOU | 2323 | C | TYR | A | 300 | 7950 | 8464 | 5731 | −478 | 23 | −1104 | C |
| ATOM | 2324 | O | TYR | A | 300 | 70.851 | 46.260 | 44.349 | 1.00 | 58.34 | | O |
| ANISOU | 2324 | O | TYR | A | 300 | 8079 | 8357 | 5731 | −556 | 105 | −1136 | O |
| ATOM | 2325 | N | HIS | A | 301 | 72.773 | 45.825 | 43.212 | 1.00 | 56.40 | | N |
| ANISOU | 2325 | N | HIS | A | 301 | 7513 | 8477 | 5439 | −475 | −107 | −1205 | N |
| ATOM | 2326 | CA | HIS | A | 301 | 73.626 | 46.801 | 43.903 | 1.00 | 57.11 | | C |
| ANISOU | 2326 | CA | HIS | A | 301 | 7501 | 8739 | 5461 | −596 | −165 | −1375 | C |
| ATOM | 2327 | CB | HIS | A | 301 | 75.059 | 46.732 | 43.361 | 1.00 | 59.14 | | C |
| ANISOU | 2327 | CB | HIS | A | 301 | 7504 | 9314 | 5652 | −570 | −312 | −1470 | C |
| ATOM | 2328 | CG | HIS | A | 301 | 76.110 | 47.255 | 44.290 | 1.00 | 63.70 | | C |
| ANISOU | 2328 | CG | HIS | A | 301 | 7979 | 10157 | 6066 | −623 | −425 | −1661 | C |
| ATOM | 2329 | ND1 | HIS | A | 301 | 76.228 | 48.609 | 44.560 | 1.00 | 65.83 | | N |
| ANISOU | 2329 | ND1 | HIS | A | 301 | 8191 | 10418 | 6405 | −895 | −355 | −1788 | N |
| ATOM | 2330 | CE1 | HIS | A | 301 | 77.266 | 48.722 | 45.371 | 1.00 | 65.55 | | C |
| ANISOU | 2330 | CE1 | HIS | A | 301 | 8051 | 10677 | 6179 | −891 | −493 | −1960 | C |
| ATOM | 2331 | NE2 | HIS | A | 301 | 77.831 | 47.537 | 45.611 | 1.00 | 65.97 | | N |
| ANISOU | 2331 | NE2 | HIS | A | 301 | 8084 | 10938 | 6042 | −605 | −652 | −1944 | N |
| ATOM | 2332 | CD2 | HIS | A | 301 | 77.115 | 46.594 | 44.913 | 1.00 | 66.03 | | C |
| ANISOU | 2332 | CD2 | HIS | A | 301 | 8212 | 10749 | 6126 | −435 | −604 | −1751 | C |
| ATOM | 2333 | C | HIS | A | 301 | 73.057 | 48.208 | 43.680 | 1.00 | 56.72 | | C |
| ANISOU | 2333 | C | HIS | A | 301 | 7429 | 8525 | 5595 | −850 | −11 | −1401 | C |
| ATOM | 2334 | O | HIS | A | 301 | 72.976 | 48.989 | 44.637 | 1.00 | 55.98 | | O |
| ANISOU | 2334 | O | HIS | A | 301 | 7417 | 8394 | 5458 | −957 | 26 | −1496 | O |
| ATOM | 2335 | N | VAL | A | 302 | 72.624 | 48.504 | 42.424 | 1.00 | 49.49 | | N |
| ANISOU | 2335 | N | VAL | A | 302 | 6430 | 7500 | 4874 | −927 | 82 | −1312 | N |
| ATOM | 2336 | CA | VAL | A | 302 | 72.001 | 49.775 | 42.060 | 1.00 | 47.77 | | C |
| ANISOU | 2336 | CA | VAL | A | 302 | 6222 | 7102 | 4825 | −1122 | 242 | −1310 | C |
| ATOM | 2337 | CB | VAL | A | 302 | 71.844 | 49.945 | 40.536 | 1.00 | 50.01 | | C |
| ANISOU | 2337 | CB | VAL | A | 302 | 6384 | 7336 | 5281 | −1160 | 303 | −1216 | C |
| ATOM | 2338 | CG1 | VAL | A | 302 | 71.129 | 51.245 | 40.184 | 1.00 | 49.27 | | C |
| ANISOU | 2338 | CG1 | VAL | A | 302 | 6349 | 7031 | 5340 | −1314 | 480 | −1196 | C |
| ATOM | 2339 | CG2 | VAL | A | 302 | 73.196 | 49.886 | 39.851 | 1.00 | 49.72 | | C |
| ANISOU | 2339 | CG2 | VAL | A | 302 | 6128 | 7545 | 5217 | −1199 | 195 | −1293 | C |
| ATOM | 2340 | C | VAL | A | 302 | 70.670 | 49.902 | 42.815 | 1.00 | 52.38 | | C |
| ANISOU | 2340 | C | VAL | A | 302 | 7023 | 7448 | 5429 | −1101 | 368 | −1245 | C |
| ATOM | 2341 | O | VAL | A | 302 | 70.469 | 50.914 | 43.479 | 1.00 | 53.45 | | O |
| ANISOU | 2341 | O | VAL | A | 302 | 7243 | 7496 | 5568 | −1223 | 449 | −1321 | O |
| ATOM | 2342 | N | LEU | A | 303 | 69.803 | 48.856 | 42.784 | 1.00 | 46.42 | | N |
| ANISOU | 2342 | N | LEU | A | 303 | 6367 | 6603 | 4668 | −952 | 385 | −1120 | N |
| ATOM | 2343 | CA | LEU | A | 303 | 68.517 | 48.816 | 43.500 | 1.00 | 44.44 | | C |
| ANISOU | 2343 | CA | LEU | A | 303 | 6303 | 6160 | 4422 | −929 | 509 | −1059 | C |
| ATOM | 2344 | CB | LEU | A | 303 | 67.924 | 47.392 | 43.457 | 1.00 | 42.80 | | C |
| ANISOU | 2344 | CB | LEU | A | 303 | 6186 | 5913 | 4163 | −781 | 497 | −945 | C |
| ATOM | 2345 | CG | LEU | A | 303 | 66.624 | 47.107 | 44.186 | 1.00 | 44.78 | | C |
| ANISOU | 2345 | CG | LEU | A | 303 | 6623 | 6000 | 4391 | −760 | 622 | −885 | C |
| ATOM | 2346 | CD1 | LEU | A | 303 | 65.497 | 47.910 | 43.633 | 1.00 | 44.01 | | C |
| ANISOU | 2346 | CD1 | LEU | A | 303 | 6490 | 5769 | 4464 | −847 | 785 | −833 | C |
| ATOM | 2347 | CD2 | LEU | A | 303 | 66.274 | 45.649 | 44.083 | 1.00 | 46.07 | | C |
| ANISOU | 2347 | CD2 | LEU | A | 303 | 6882 | 6139 | 4481 | −648 | 604 | −794 | C |
| ATOM | 2348 | C | LEU | A | 303 | 68.680 | 49.318 | 44.945 | 1.00 | 52.07 | | C |
| ANISOU | 2348 | C | LEU | A | 303 | 7403 | 7124 | 5257 | −964 | 507 | −1169 | C |
| ATOM | 2349 | O | LEU | A | 303 | 67.900 | 50.171 | 45.386 | 1.00 | 53.44 | | O |
| ANISOU | 2349 | O | LEU | A | 303 | 7677 | 7146 | 5483 | −1039 | 641 | −1175 | O |
| ATOM | 2350 | N | GLY | A | 304 | 69.726 | 48.841 | 45.629 | 1.00 | 48.62 | | N |
| ANISOU | 2350 | N | GLY | A | 304 | 6958 | 6871 | 4644 | −901 | 355 | −1263 | N |
| ATOM | 2351 | CA | GLY | A | 304 | 70.051 | 49.263 | 46.986 | 1.00 | 48.29 | | C |
| ANISOU | 2351 | CA | GLY | A | 304 | 7026 | 6871 | 4452 | −927 | 325 | −1386 | C |
| ATOM | 2352 | C | GLY | A | 304 | 70.315 | 50.753 | 47.032 | 1.00 | 52.85 | | C |
| ANISOU | 2352 | C | GLY | A | 304 | 7552 | 7426 | 5101 | −1139 | 389 | −1504 | C |
| ATOM | 2353 | O | GLY | A | 304 | 69.640 | 51.488 | 47.758 | 1.00 | 52.44 | | O |
| ANISOU | 2353 | O | GLY | A | 304 | 7649 | 7221 | 5054 | −1208 | 503 | −1532 | O |
| ATOM | 2354 | N | LEU | A | 305 | 71.230 | 51.208 | 46.165 | 1.00 | 50.16 | | N |
| ANISOU | 2354 | N | LEU | A | 305 | 7015 | 7221 | 4824 | −1249 | 336 | −1567 | N |
| ATOM | 2355 | CA | LEU | A | 305 | 71.634 | 52.608 | 46.016 | 1.00 | 49.78 | | C |
| ANISOU | 2355 | CA | LEU | A | 305 | 6918 | 7154 | 4841 | −1484 | 404 | −1686 | C |
| ATOM | 2356 | CB | LEU | A | 305 | 72.700 | 52.712 | 44.909 | 1.00 | 49.62 | | C |
| ANISOU | 2356 | CB | LEU | A | 305 | 6660 | 7319 | 4876 | −1572 | 333 | −1728 | C |
| ATOM | 2357 | CG | LEU | A | 305 | 74.181 | 52.677 | 45.315 | 1.00 | 53.76 | | C |
| ANISOU | 2357 | CG | LEU | A | 305 | 7014 | 8173 | 5240 | −1632 | 164 | −1908 | C |
| ATOM | 2358 | CD1 | LEU | A | 305 | 74.495 | 51.553 | 46.243 | 1.00 | 53.15 | | C |
| ANISOU | 2358 | CD1 | LEU | A | 305 | 6962 | 8273 | 4961 | −1407 | 1 | −1922 | C |
| ATOM | 2359 | CD2 | LEU | A | 305 | 75.044 | 52.491 | 44.098 | 1.00 | 58.66 | | C |
| ANISOU | 2359 | CD2 | LEU | A | 305 | 7391 | 8975 | 5922 | −1670 | 106 | −1909 | C |
| ATOM | 2360 | C | LEU | A | 305 | 70.430 | 53.538 | 45.738 | 1.00 | 52.64 | | C |
| ANISOU | 2360 | C | LEU | A | 305 | 7419 | 7221 | 5362 | −1560 | 614 | −1608 | C |
| ATOM | 2361 | O | LEU | A | 305 | 70.325 | 54.603 | 46.355 | 1.00 | 53.27 | | O |
| ANISOU | 2361 | O | LEU | A | 305 | 7618 | 7195 | 5427 | −1696 | 705 | −1701 | O |
| ATOM | 2362 | N | VAL | A | 306 | 69.506 | 53.099 | 44.864 | 1.00 | 46.49 | | N |
| ANISOU | 2362 | N | VAL | A | 306 | 6633 | 6320 | 4713 | −1454 | 688 | −1442 | N |
| ATOM | 2363 | CA | VAL | A | 306 | 68.284 | 53.817 | 44.505 | 1.00 | 45.34 | | C |
| ANISOU | 2363 | CA | VAL | A | 306 | 6590 | 5935 | 4702 | −1465 | 875 | −1350 | C |
| ATOM | 2364 | CB | VAL | A | 306 | 67.569 | 53.152 | 43.295 | 1.00 | 48.03 | | C |
| ANISOU | 2364 | CB | VAL | A | 306 | 6840 | 6239 | 5171 | −1350 | 905 | −1186 | C |
| ATOM | 2365 | CG1 | VAL | A | 306 | 66.139 | 53.667 | 43.124 | 1.00 | 47.61 | | C |
| ANISOU | 2365 | CG1 | VAL | A | 306 | 6887 | 5985 | 5216 | −1301 | 1082 | −1087 | C |
| ATOM | 2366 | CG2 | VAL | A | 306 | 68.370 | 53.359 | 42.016 | 1.00 | 47.12 | | C |
| ANISOU | 2366 | CG2 | VAL | A | 306 | 6556 | 6204 | 5145 | −1421 | 868 | −1183 | C |
| ATOM | 2367 | C | VAL | A | 306 | 67.384 | 53.951 | 45.734 | 1.00 | 50.48 | | C |
| ANISOU | 2367 | C | VAL | A | 306 | 7442 | 6456 | 5282 | −1418 | 960 | −1357 | C |
| ATOM | 2368 | O | VAL | A | 306 | 66.930 | 55.058 | 46.020 | 1.00 | 50.67 | | O |
| ANISOU | 2368 | O | VAL | A | 306 | 7589 | 6325 | 5338 | −1497 | 1095 | −1394 | O |
| ATOM | 2369 | N | HIS | A | 307 | 67.162 | 52.837 | 46.474 | 1.00 | 47.06 | | N |
| ANISOU | 2369 | N | HIS | A | 307 | 7063 | 6082 | 4737 | −1289 | 889 | −1326 | N |
| ATOM | 2370 | CA | HIS | A | 307 | 66.334 | 52.792 | 47.682 | 1.00 | 46.89 | | C |
| ANISOU | 2370 | CA | HIS | A | 307 | 7233 | 5957 | 4627 | −1234 | 966 | −1327 | C |
| ATOM | 2371 | CB | HIS | A | 307 | 66.068 | 51.345 | 48.100 | 1.00 | 47.60 | | C |
| ANISOU | 2371 | CB | HIS | A | 307 | 7371 | 6101 | 4615 | −1084 | 900 | −1252 | C |
| ATOM | 2372 | CG | HIS | A | 307 | 65.026 | 50.636 | 47.290 | 1.00 | 50.88 | | C |
| ANISOU | 2372 | CG | HIS | A | 307 | 7749 | 6444 | 5141 | −1010 | 980 | −1100 | C |
| ATOM | 2373 | ND1 | HIS | A | 307 | 64.549 | 49.394 | 47.669 | 1.00 | 52.69 | | N |
| ANISOU | 2373 | ND1 | HIS | A | 307 | 8073 | 6660 | 5285 | −910 | 984 | −1026 | N |
| ATOM | 2374 | CE1 | HIS | A | 307 | 63.657 | 49.053 | 46.755 | 1.00 | 52.14 | | C |
| ANISOU | 2374 | CE1 | HIS | A | 307 | 7928 | 6542 | 5342 | −897 | 1063 | −916 | C |
| ATOM | 2375 | NE2 | HIS | A | 307 | 63.531 | 49.996 | 45.824 | 1.00 | 52.40 | | N |
| ANISOU | 2375 | NE2 | HIS | A | 307 | 7825 | 6557 | 5527 | −955 | 1105 | −908 | N |
| ATOM | 2376 | CD2 | HIS | A | 307 | 64.398 | 51.013 | 46.154 | 1.00 | 52.58 | | C |
| ANISOU | 2376 | CD2 | HIS | A | 307 | 7849 | 6602 | 5526 | −1032 | 1062 | −1020 | C |
| ATOM | 2377 | C | HIS | A | 307 | 66.981 | 53.556 | 48.821 | 1.00 | 52.45 | | C |
| ANISOU | 2377 | C | HIS | A | 307 | 8044 | 6682 | 5203 | −1331 | 942 | −1487 | C |
| ATOM | 2378 | O | HIS | A | 307 | 66.296 | 54.017 | 49.730 | 1.00 | 52.99 | | O |
| ANISOU | 2378 | O | HIS | A | 307 | 8284 | 6624 | 5226 | −1328 | 1048 | −1506 | O |
| ATOM | 2379 | N | ALA | A | 308 | 68.296 | 53.701 | 48.778 | 1.00 | 49.75 | | N |
| ANISOU | 2379 | N | ALA | A | 308 | 7594 | 6516 | 4793 | −1421 | 804 | −1611 | N |
| ATOM | 2380 | CA | ALA | A | 308 | 69.004 | 54.481 | 49.783 | 1.00 | 50.05 | | C |
| ANISOU | 2380 | CA | ALA | A | 308 | 7706 | 6607 | 4703 | −1548 | 771 | −1790 | C |
| ATOM | 2381 | CB | ALA | A | 308 | 70.464 | 54.054 | 49.833 | 1.00 | 51.06 | | C |
| ANISOU | 2381 | CB | ALA | A | 308 | 7669 | 7030 | 4702 | −1573 | 564 | −1913 | C |
| ATOM | 2382 | C | ALA | A | 308 | 68.881 | 56.001 | 49.454 | 1.00 | 53.43 | | C |
| ANISOU | 2382 | C | ALA | A | 308 | 8189 | 6874 | 5239 | −1747 | 920 | −1854 | C |
| ATOM | 2383 | O | ALA | A | 308 | 68.939 | 56.833 | 50.358 | 1.00 | 52.77 | | O |
| ANISOU | 2383 | O | ALA | A | 308 | 8249 | 6727 | 5073 | −1859 | 969 | −1980 | O |
| ATOM | 2384 | N | LEU | A | 309 | 68.689 | 56.350 | 48.163 | 1.00 | 49.58 | | N |
| ANISOU | 2384 | N | LEU | A | 309 | 7610 | 6308 | 4920 | −1782 | 997 | −1767 | N |
| ATOM | 2385 | CA | LEU | A | 309 | 68.552 | 57.730 | 47.690 | 1.00 | 48.71 | | C |
| ANISOU | 2385 | CA | LEU | A | 309 | 7583 | 6019 | 4905 | −1942 | 1154 | −1803 | C |
| ATOM | 2386 | CB | LEU | A | 309 | 69.189 | 57.887 | 46.299 | 1.00 | 48.75 | | C |
| ANISOU | 2386 | CB | LEU | A | 309 | 7411 | 6085 | 5025 | −2028 | 1137 | −1776 | C |
| ATOM | 2387 | CG | LEU | A | 309 | 68.733 | 59.044 | 45.418 | 1.00 | 53.52 | | C |
| ANISOU | 2387 | CG | LEU | A | 309 | 8114 | 6460 | 5759 | −2105 | 1326 | −1726 | C |
| ATOM | 2388 | CD1 | LEU | A | 309 | 69.350 | 60.349 | 45.860 | 1.00 | 53.92 | | C |
| ANISOU | 2388 | CD1 | LEU | A | 309 | 8320 | 6414 | 5752 | −2351 | 1416 | −1895 | C |
| ATOM | 2389 | CD2 | LEU | A | 309 | 69.099 | 58.785 | 43.985 | 1.00 | 55.08 | | C |
| ANISOU | 2389 | CD2 | LEU | A | 309 | 8127 | 6729 | 6072 | −2107 | 1299 | −1644 | C |
| ATOM | 2390 | C | LEU | A | 309 | 67.096 | 58.235 | 47.739 | 1.00 | 51.62 | | C |
| ANISOU | 2390 | C | LEU | A | 309 | 8139 | 6125 | 5349 | −1837 | 1350 | −1692 | C |
| ATOM | 2391 | O | LEU | A | 309 | 66.875 | 59.314 | 48.283 | 1.00 | 51.57 | | O |
| ANISOU | 2391 | O | LEU | A | 309 | 8327 | 5957 | 5310 | −1924 | 1473 | −1770 | O |
| ATOM | 2392 | N | TRP | A | 310 | 66.118 | 57.471 | 47.191 | 1.00 | 47.17 | | N |
| ANISOU | 2392 | N | TRP | A | 310 | 7517 | 5533 | 4873 | −1651 | 1381 | −1522 | N |
| ATOM | 2393 | CA | TRP | A | 310 | 64.687 | 57.822 | 47.228 | 1.00 | 46.36 | | C |
| ANISOU | 2393 | CA | TRP | A | 310 | 7541 | 5246 | 4828 | −1522 | 1556 | −1416 | C |
| ATOM | 2394 | CB | TRP | A | 310 | 64.126 | 58.106 | 45.842 | 1.00 | 45.42 | | C |
| ANISOU | 2394 | CB | TRP | A | 310 | 7347 | 5048 | 4863 | −1460 | 1650 | −1291 | C |
| ATOM | 2395 | CG | TRP | A | 310 | 64.823 | 59.201 | 45.104 | 1.00 | 47.45 | | C |
| ANISOU | 2395 | CG | TRP | A | 310 | 7634 | 5222 | 5171 | −1606 | 1703 | −1346 | C |
| ATOM | 2396 | CD1 | TRP | A | 310 | 64.971 | 60.499 | 45.498 | 1.00 | 50.51 | | C |
| ANISOU | 2396 | CD1 | TRP | A | 310 | 8234 | 5434 | 5523 | −1720 | 1831 | −1440 | C |
| ATOM | 2397 | NE1 | TRP | A | 310 | 65.625 | 61.218 | 44.523 | 1.00 | 50.31 | | N |
| ANISOU | 2397 | NE1 | TRP | A | 310 | 8201 | 5354 | 5560 | −1849 | 1874 | −1458 | N |
| ATOM | 2398 | CE2 | TRP | A | 310 | 65.899 | 60.387 | 43.465 | 1.00 | 51.94 | | C |
| ANISOU | 2398 | CE2 | TRP | A | 310 | 8173 | 5714 | 5850 | −1805 | 1767 | −1371 | C |
| ATOM | 2399 | CD2 | TRP | A | 310 | 65.400 | 59.108 | 43.795 | 1.00 | 47.71 | | C |
| ANISOU | 2399 | CD2 | TRP | A | 310 | 7509 | 5319 | 5299 | −1650 | 1658 | −1300 | C |
| ATOM | 2400 | CE3 | TRP | A | 310 | 65.549 | 58.063 | 42.870 | 1.00 | 49.21 | | C |
| ANISOU | 2400 | CE3 | TRP | A | 310 | 7479 | 5666 | 5553 | −1578 | 1543 | −1210 | C |
| ATOM | 2401 | CZ3 | TRP | A | 310 | 66.169 | 58.325 | 41.655 | 1.00 | 51.17 | | C |
| ANISOU | 2401 | CZ3 | TRP | A | 310 | 7623 | 5937 | 5882 | −1648 | 1536 | −1188 | C |
| ATOM | 2402 | CH2 | TRP | A | 310 | 66.652 | 59.603 | 41.350 | 1.00 | 51.93 | | C |
| ANISOU | 2402 | CH2 | TRP | A | 310 | 7842 | 5896 | 5993 | −1802 | 1650 | −1254 | C |
| ATOM | 2403 | CZ2 | TRP | A | 310 | 66.544 | 60.646 | 42.249 | 1.00 | 51.74 | | C |
| ANISOU | 2403 | CZ2 | TRP | A | 310 | 8053 | 5704 | 5902 | −1891 | 1769 | −1350 | C |
| ATOM | 2404 | C | TRP | A | 310 | 63.830 | 56.766 | 47.924 | 1.00 | 47.30 | | C |
| ANISOU | 2404 | C | TRP | A | 310 | 7670 | 5408 | 4893 | −1367 | 1541 | −1340 | C |
| ATOM | 2405 | O | TRP | A | 310 | 64.128 | 55.574 | 47.827 | 1.00 | 47.08 | | O |
| ANISOU | 2405 | O | TRP | A | 310 | 7524 | 5526 | 4838 | −1315 | 1411 | −1300 | O |
| ATOM | 2406 | N | GLN | A | 311 | 62.736 | 57.204 | 48.576 | 1.00 | 40.88 | | N |
| ANISOU | 2406 | N | GLN | A | 311 | 7012 | 4461 | 4058 | −1292 | 1690 | −1317 | N |
| ATOM | 2407 | CA | GLN | A | 311 | 61.823 | 56.347 | 49.336 | 1.00 | 39.93 | | C |
| ANISOU | 2407 | CA | GLN | A | 311 | 6934 | 4361 | 3876 | −1170 | 1721 | −1257 | C |
| ATOM | 2408 | CB | GLN | A | 311 | 60.839 | 57.202 | 50.142 | 1.00 | 41.48 | | C |
| ANISOU | 2408 | CB | GLN | A | 311 | 7316 | 4407 | 4038 | −1114 | 1902 | −1269 | C |
| ATOM | 2409 | CG | GLN | A | 311 | 60.266 | 56.413 | 51.303 | 1.00 | 48.28 | | C |
| ANISOU | 2409 | CG | GLN | A | 311 | 8251 | 5304 | 4789 | −1038 | 1920 | −1252 | C |
| ATOM | 2410 | CD | GLN | A | 311 | 58.867 | 56.790 | 51.700 | 1.00 | 53.82 | | C |
| ANISOU | 2410 | CD | GLN | A | 311 | 9054 | 5909 | 5485 | −933 | 2114 | −1210 | C |
| ATOM | 2411 | OE1 | GLN | A | 311 | 58.573 | 57.972 | 51.974 | 1.00 | 47.11 | | O |
| ANISOU | 2411 | OE1 | GLN | A | 311 | 8350 | 4926 | 4623 | −927 | 2230 | −1263 | O |
| ATOM | 2412 | NE2 | GLN | A | 311 | 57.984 | 55.767 | 51.760 | 1.00 | 33.04 | | N |
| ANISOU | 2412 | NE2 | GLN | A | 311 | 6356 | 3348 | 2848 | −852 | 2160 | −1120 | N |
| ATOM | 2413 | C | GLN | A | 311 | 61.049 | 55.302 | 48.516 | 1.00 | 42.04 | | C |
| ANISOU | 2413 | C | GLN | A | 311 | 7047 | 4699 | 4227 | −1064 | 1722 | −1113 | C |
| ATOM | 2414 | O | GLN | A | 311 | 60.169 | 55.696 | 47.764 | 1.00 | 41.48 | | O |
| ANISOU | 2414 | O | GLN | A | 311 | 6918 | 4577 | 4267 | −1002 | 1831 | −1034 | O |
| ATOM | 2415 | N | PRO | A | 312 | 61.255 | 53.976 | 48.726 | 1.00 | 38.60 | | N |
| ANISOU | 2415 | N | PRO | A | 312 | 6561 | 4374 | 3729 | −1032 | 1618 | −1079 | N |
| ATOM | 2416 | CA | PRO | A | 312 | 60.500 | 52.982 | 47.933 | 1.00 | 38.67 | | C |
| ANISOU | 2416 | CA | PRO | A | 312 | 6440 | 4436 | 3818 | −964 | 1637 | −954 | C |
| ATOM | 2417 | CB | PRO | A | 312 | 61.288 | 51.686 | 48.135 | 1.00 | 40.20 | | C |
| ANISOU | 2417 | CB | PRO | A | 312 | 6612 | 4738 | 3924 | −962 | 1476 | −953 | C |
| ATOM | 2418 | CG | PRO | A | 312 | 61.997 | 51.861 | 49.402 | 1.00 | 44.45 | | C |
| ANISOU | 2418 | CG | PRO | A | 312 | 7300 | 5285 | 4302 | −983 | 1408 | −1060 | C |
| ATOM | 2419 | CD | PRO | A | 312 | 62.246 | 53.322 | 49.601 | 1.00 | 40.13 | | C |
| ANISOU | 2419 | CD | PRO | A | 312 | 6822 | 4655 | 3769 | −1053 | 1475 | −1154 | C |
| ATOM | 2420 | C | PRO | A | 312 | 59.021 | 52.814 | 48.299 | 1.00 | 43.57 | | C |
| ANISOU | 2420 | C | PRO | A | 312 | 7100 | 5019 | 4435 | −894 | 1798 | −891 | C |
| ATOM | 2421 | O | PRO | A | 312 | 58.701 | 52.588 | 49.476 | 1.00 | 44.57 | | O |
| ANISOU | 2421 | O | PRO | A | 312 | 7371 | 5118 | 4444 | −883 | 1845 | −922 | O |
| ATOM | 2422 | N | ILE | A | 313 | 58.119 | 52.920 | 47.283 | 1.00 | 39.10 | | N |
| ANISOU | 2422 | N | ILE | A | 313 | 6394 | 4473 | 3991 | −844 | 1881 | −806 | N |
| ATOM | 2423 | CA | ILE | A | 313 | 56.666 | 52.770 | 47.469 | 1.00 | 38.25 | | C |
| ANISOU | 2423 | CA | ILE | A | 313 | 6258 | 4383 | 3891 | −780 | 2037 | −750 | C |
| ATOM | 2424 | CB | ILE | A | 313 | 55.804 | 53.346 | 46.298 | 1.00 | 40.55 | | C |
| ANISOU | 2424 | CB | ILE | A | 313 | 6388 | 4708 | 4312 | −698 | 2125 | −684 | C |
| ATOM | 2425 | CG1 | ILE | A | 313 | 56.294 | 54.729 | 45.839 | 1.00 | 39.74 | | C |
| ANISOU | 2425 | CG1 | ILE | A | 313 | 6335 | 4502 | 4262 | −670 | 2137 | −714 | C |
| ATOM | 2426 | CD1 | ILE | A | 313 | 55.433 | 55.421 | 44.907 | 1.00 | 43.72 | | C |
| ANISOU | 2426 | CD1 | ILE | A | 313 | 6743 | 5016 | 4854 | −553 | 2233 | −653 | C |
| ATOM | 2427 | CG2 | ILE | A | 313 | 54.305 | 53.347 | 46.677 | 1.00 | 41.60 | | C |
| ANISOU | 2427 | CG2 | ILE | A | 313 | 6490 | 4893 | 4425 | −619 | 2297 | −656 | C |
| ATOM | 2428 | C | ILE | A | 313 | 56.376 | 51.275 | 47.666 | 1.00 | 42.46 | | C |
| ANISOU | 2428 | C | ILE | A | 313 | 6773 | 4994 | 4367 | −815 | 2012 | −709 | C |
| ATOM | 2429 | O | ILE | A | 313 | 56.777 | 50.484 | 46.793 | 1.00 | 41.96 | | O |
| ANISOU | 2429 | O | ILE | A | 313 | 6603 | 4992 | 4348 | −842 | 1912 | −666 | O |
| ATOM | 2430 | N | PRO | A | 314 | 55.683 | 50.883 | 48.782 | 1.00 | 38.09 | | N |
| ANISOU | 2430 | N | PRO | A | 314 | 6344 | 4424 | 3705 | −819 | 2113 | −722 | N |
| ATOM | 2431 | CA | PRO | A | 314 | 55.385 | 49.461 | 49.034 | 1.00 | 37.86 | | C |
| ANISOU | 2431 | CA | PRO | A | 314 | 6350 | 4437 | 3599 | −872 | 2122 | −686 | C |
| ATOM | 2432 | CB | PRO | A | 314 | 54.309 | 49.518 | 50.118 | 1.00 | 39.25 | | C |
| ANISOU | 2432 | CB | PRO | A | 314 | 6615 | 4600 | 3697 | −865 | 2303 | −698 | C |
| ATOM | 2433 | CG | PRO | A | 314 | 54.589 | 50.720 | 50.843 | 1.00 | 43.33 | | C |
| ANISOU | 2433 | CG | PRO | A | 314 | 7254 | 5032 | 4176 | −813 | 2314 | −765 | C |
| ATOM | 2434 | CD | PRO | A | 314 | 55.209 | 51.712 | 49.905 | 1.00 | 39.38 | | C |
| ANISOU | 2434 | CD | PRO | A | 314 | 6652 | 4515 | 3797 | −781 | 2234 | −775 | C |
| ATOM | 2435 | C | PRO | A | 314 | 54.913 | 48.625 | 47.841 | 1.00 | 44.38 | | C |
| ANISOU | 2435 | C | PRO | A | 314 | 6997 | 5355 | 4511 | −908 | 2112 | −616 | C |
| ATOM | 2436 | O | PRO | A | 314 | 53.940 | 48.963 | 47.150 | 1.00 | 43.04 | | O |
| ANISOU | 2436 | O | PRO | A | 314 | 6653 | 5263 | 4436 | −887 | 2207 | −585 | O |
| ATOM | 2437 | N | GLY | A | 315 | 55.651 | 47.538 | 47.618 | 1.00 | 43.53 | | N |
| ANISOU | 2437 | N | GLY | A | 315 | 6940 | 5244 | 4354 | −947 | 1991 | −599 | N |
| ATOM | 2438 | CA | GLY | A | 315 | 55.402 | 46.526 | 46.601 | 1.00 | 43.30 | | C |
| ANISOU | 2438 | CA | GLY | A | 315 | 6802 | 5277 | 4371 | −998 | 1961 | −542 | C |
| ATOM | 2439 | C | GLY | A | 315 | 55.373 | 47.064 | 45.204 | 1.00 | 47.60 | | C |
| ANISOU | 2439 | C | GLY | A | 315 | 7114 | 5896 | 5074 | −968 | 1919 | −513 | C |
| ATOM | 2440 | O | GLY | A | 315 | 54.329 | 47.047 | 44.550 | 1.00 | 49.16 | | O |
| ANISOU | 2440 | O | GLY | A | 315 | 7152 | 6183 | 5344 | −982 | 2016 | −484 | O |
| ATOM | 2441 | N | ARG | A | 316 | 56.520 | 47.564 | 44.754 | 1.00 | 42.14 | | N |
| ANISOU | 2441 | N | ARG | A | 316 | 6399 | 5185 | 4427 | −926 | 1780 | −525 | N |
| ATOM | 2442 | CA | ARG | A | 316 | 56.692 | 48.124 | 43.415 | 1.00 | 39.07 | | C |
| ANISOU | 2442 | CA | ARG | A | 316 | 5820 | 4847 | 4177 | −892 | 1732 | −495 | C |
| ATOM | 2443 | CB | ARG | A | 316 | 56.601 | 49.664 | 43.424 | 1.00 | 35.97 | | C |
| ANISOU | 2443 | CB | ARG | A | 316 | 5401 | 4415 | 3850 | −830 | 1794 | −520 | C |
| ATOM | 2444 | CG | ARG | A | 316 | 55.211 | 50.227 | 43.819 | 1.00 | 43.01 | | C |
| ANISOU | 2444 | CG | ARG | A | 316 | 6265 | 5328 | 4749 | −782 | 1972 | −513 | C |
| ATOM | 2445 | CD | ARG | A | 316 | 54.104 | 49.945 | 42.823 | 1.00 | 51.25 | | C |
| ANISOU | 2445 | CD | ARG | A | 316 | 7104 | 6505 | 5864 | −760 | 2034 | −458 | C |
| ATOM | 2446 | NE | ARG | A | 316 | 54.542 | 50.303 | 41.474 | 1.00 | 68.40 | | N |
| ANISOU | 2446 | NE | ARG | A | 316 | 9142 | 8706 | 8141 | −711 | 1954 | −420 | N |
| ATOM | 2447 | CZ | ARG | A | 316 | 53.731 | 50.651 | 40.487 | 1.00 | 76.47 | | C |
| ANISOU | 2447 | CZ | ARG | A | 316 | 9989 | 9831 | 9236 | −628 | 2007 | −379 | C |
| ATOM | 2448 | NH1 | ARG | A | 316 | 54.227 | 50.984 | 39.301 | 1.00 | 47.84 | | N |
| ANISOU | 2448 | NH1 | ARG | A | 316 | 6273 | 6214 | 5691 | −579 | 1932 | −342 | N |
| ATOM | 2449 | NH2 | ARG | A | 316 | 52.417 | 50.682 | 40.678 | 1.00 | 67.78 | | N |
| ANISOU | 2449 | NH2 | ARG | A | 316 | 8797 | 8841 | 8115 | −586 | 2137 | −378 | N |
| ATOM | 2450 | C | ARG | A | 316 | 57.993 | 47.643 | 42.833 | 1.00 | 40.03 | | C |
| ANISOU | 2450 | C | ARG | A | 316 | 5936 | 4975 | 4297 | −894 | 1561 | −495 | C |
| ATOM | 2451 | O | ARG | A | 316 | 58.414 | 48.145 | 41.791 | 1.00 | 41.62 | | O |
| ANISOU | 2451 | O | ARG | A | 316 | 6011 | 5205 | 4599 | −870 | 1505 | −480 | O |
| ATOM | 2452 | N | VAL | A | 317 | 58.619 | 46.645 | 43.475 | 1.00 | 33.91 | | N |
| ANISOU | 2452 | N | VAL | A | 317 | 5305 | 4180 | 3397 | −907 | 1484 | −509 | N |
| ATOM | 2453 | CA | VAL | A | 317 | 59.841 | 46.033 | 42.957 | 1.00 | 33.46 | | C |
| ANISOU | 2453 | CA | VAL | A | 317 | 5242 | 4156 | 3313 | −880 | 1321 | −508 | C |
| ATOM | 2454 | CB | VAL | A | 317 | 60.916 | 45.646 | 44.020 | 1.00 | 36.06 | | C |
| ANISOU | 2454 | CB | VAL | A | 317 | 5744 | 4475 | 3483 | −839 | 1213 | −565 | C |
| ATOM | 2455 | CG1 | VAL | A | 317 | 62.144 | 44.983 | 43.375 | 1.00 | 34.55 | | C |
| ANISOU | 2455 | CG1 | VAL | A | 317 | 5505 | 4360 | 3263 | −783 | 1043 | −565 | C |
| ATOM | 2456 | CG2 | VAL | A | 317 | 61.340 | 46.877 | 44.833 | 1.00 | 36.07 | | C |
| ANISOU | 2456 | CG2 | VAL | A | 317 | 5778 | 4458 | 3470 | −847 | 1221 | −646 | C |
| ATOM | 2457 | C | VAL | A | 317 | 59.398 | 44.878 | 42.059 | 1.00 | 39.13 | | C |
| ANISOU | 2457 | C | VAL | A | 317 | 5911 | 4905 | 4052 | −901 | 1319 | −443 | C |
| ATOM | 2458 | O | VAL | A | 317 | 58.634 | 43.995 | 42.482 | 1.00 | 41.64 | | O |
| ANISOU | 2458 | O | VAL | A | 317 | 6330 | 5192 | 4300 | −949 | 1405 | −422 | O |
| ATOM | 2459 | N | LYS | A | 318 | 59.799 | 44.951 | 40.796 | 1.00 | 31.92 | | N |
| ANISOU | 2459 | N | LYS | A | 318 | 4840 | 4050 | 3238 | −883 | 1243 | −415 | N |
| ATOM | 2460 | CA | LYS | A | 318 | 59.453 | 43.957 | 39.813 | 1.00 | 30.62 | | C |
| ANISOU | 2460 | CA | LYS | A | 318 | 4616 | 3920 | 3098 | −905 | 1231 | −362 | C |
| ATOM | 2461 | CB | LYS | A | 318 | 58.585 | 44.591 | 38.708 | 1.00 | 31.94 | | C |
| ANISOU | 2461 | CB | LYS | A | 318 | 4569 | 4152 | 3413 | −921 | 1297 | −329 | C |
| ATOM | 2462 | CG | LYS | A | 318 | 57.257 | 45.185 | 39.207 | 1.00 | 41.17 | | C |
| ANISOU | 2462 | CG | LYS | A | 318 | 5703 | 5332 | 4606 | −953 | 1457 | −336 | C |
| ATOM | 2463 | CD | LYS | A | 318 | 56.724 | 46.301 | 38.295 | 1.00 | 55.57 | | C |
| ANISOU | 2463 | CD | LYS | A | 318 | 7330 | 7224 | 6561 | −897 | 1504 | −315 | C |
| ATOM | 2464 | CE | LYS | A | 318 | 56.222 | 47.520 | 39.046 | 1.00 | 71.14 | | C |
| ANISOU | 2464 | CE | LYS | A | 318 | 9324 | 9162 | 8545 | −850 | 1612 | −341 | C |
| ATOM | 2465 | NZ | LYS | A | 318 | 57.274 | 48.571 | 39.213 | 1.00 | 77.69 | | N |
| ANISOU | 2465 | NZ | LYS | A | 318 | 10211 | 9910 | 9397 | −805 | 1557 | −367 | N |
| ATOM | 2466 | C | LYS | A | 318 | 60.788 | 43.414 | 39.296 | 1.00 | 34.60 | | C |
| ANISOU | 2466 | C | LYS | A | 318 | 5131 | 4452 | 3563 | −840 | 1070 | −359 | C |
| ATOM | 2467 | O | LYS | A | 318 | 61.566 | 44.138 | 38.657 | 1.00 | 33.20 | | O |
| ANISOU | 2467 | O | LYS | A | 318 | 4825 | 4328 | 3463 | −806 | 993 | −370 | O |
| ATOM | 2468 | N | ASP | A | 319 | 61.107 | 42.170 | 39.686 | 1.00 | 31.73 | | N |
| ANISOU | 2468 | N | ASP | A | 319 | 4947 | 4050 | 3059 | −815 | 1027 | −350 | N |
| ATOM | 2469 | CA | ASP | A | 319 | 62.364 | 41.561 | 39.276 | 1.00 | 31.79 | | C |
| ANISOU | 2469 | CA | ASP | A | 319 | 4985 | 4097 | 2997 | −716 | 877 | −349 | C |
| ATOM | 2470 | CB | ASP | A | 319 | 63.026 | 40.803 | 40.439 | 1.00 | 34.80 | | C |
| ANISOU | 2470 | CB | ASP | A | 319 | 5611 | 4437 | 3175 | −629 | 824 | −373 | C |
| ATOM | 2471 | CG | ASP | A | 319 | 64.398 | 40.201 | 40.161 | 1.00 | 49.18 | | C |
| ANISOU | 2471 | CG | ASP | A | 319 | 7461 | 6334 | 4892 | −481 | 660 | −382 | C |
| ATOM | 2472 | OD1 | ASP | A | 319 | 64.976 | 40.484 | 39.078 | 1.00 | 48.72 | | O |
| ANISOU | 2472 | OD1 | ASP | A | 319 | 7205 | 6375 | 4932 | −460 | 579 | −382 | O |
| ATOM | 2473 | OD2 | ASP | A | 319 | 64.901 | 39.461 | 41.028 | 1.00 | 57.32 | | O |
| ANISOU | 2473 | OD2 | ASP | A | 319 | 8713 | 7334 | 5732 | −373 | 616 | −390 | O |
| ATOM | 2474 | C | ASP | A | 319 | 62.149 | 40.679 | 38.076 | 1.00 | 34.60 | | C |
| ANISOU | 2474 | C | ASP | A | 319 | 5301 | 4460 | 3385 | −726 | 864 | −295 | C |
| ATOM | 2475 | O | ASP | A | 319 | 61.601 | 39.570 | 38.186 | 1.00 | 33.70 | | O |
| ANISOU | 2475 | O | ASP | A | 319 | 5352 | 4271 | 3183 | −763 | 919 | −270 | O |
| ATOM | 2476 | N | TYR | A | 320 | 62.578 | 41.211 | 36.909 | 1.00 | 31.14 | | N |
| ANISOU | 2476 | N | TYR | A | 320 | 4656 | 4106 | 3070 | −707 | 800 | −283 | N |
| ATOM | 2477 | CA | TYR | A | 320 | 62.504 | 40.597 | 35.583 | 1.00 | 29.83 | | C |
| ANISOU | 2477 | CA | TYR | A | 320 | 4408 | 3970 | 2955 | −707 | 771 | −238 | C |
| ATOM | 2478 | CB | TYR | A | 320 | 62.043 | 41.601 | 34.527 | 1.00 | 28.78 | | C |
| ANISOU | 2478 | CB | TYR | A | 320 | 4033 | 3904 | 2997 | −746 | 804 | −220 | C |
| ATOM | 2479 | CG | TYR | A | 320 | 60.668 | 42.150 | 34.775 | 1.00 | 27.63 | | C |
| ANISOU | 2479 | CG | TYR | A | 320 | 3833 | 3745 | 2920 | −834 | 944 | −218 | C |
| ATOM | 2480 | CD1 | TYR | A | 320 | 59.573 | 41.305 | 34.894 | 1.00 | 29.80 | | C |
| ANISOU | 2480 | CD1 | TYR | A | 320 | 4169 | 3999 | 3155 | −923 | 1034 | −209 | C |
| ATOM | 2481 | CE1 | TYR | A | 320 | 58.302 | 41.804 | 35.159 | 1.00 | 31.78 | | C |
| ANISOU | 2481 | CE1 | TYR | A | 320 | 4343 | 4279 | 3454 | −996 | 1165 | −218 | C |
| ATOM | 2482 | CZ | TYR | A | 320 | 58.110 | 43.171 | 35.271 | 1.00 | 38.86 | | C |
| ANISOU | 2482 | CZ | TYR | A | 320 | 5123 | 5205 | 4437 | −951 | 1206 | −226 | C |
| ATOM | 2483 | OH | TYR | A | 320 | 56.841 | 43.668 | 35.476 | 1.00 | 39.95 | | O |
| ANISOU | 2483 | OH | TYR | A | 320 | 5174 | 5394 | 4610 | −989 | 1335 | −234 | O |
| ATOM | 2484 | CE2 | TYR | A | 320 | 59.191 | 44.032 | 35.143 | 1.00 | 29.03 | | C |
| ANISOU | 2484 | CE2 | TYR | A | 320 | 3852 | 3945 | 3232 | −871 | 1125 | −231 | C |
| ATOM | 2485 | CD2 | TYR | A | 320 | 60.459 | 43.515 | 34.899 | 1.00 | 27.82 | | C |
| ANISOU | 2485 | CD2 | TYR | A | 320 | 3752 | 3785 | 3034 | −828 | 995 | −232 | C |
| ATOM | 2486 | C | TYR | A | 320 | 63.815 | 39.968 | 35.178 | 1.00 | 37.57 | | C |
| ANISOU | 2486 | C | TYR | A | 320 | 5426 | 4996 | 3854 | −583 | 629 | −236 | C |
| ATOM | 2487 | O | TYR | A | 320 | 63.892 | 39.326 | 34.133 | 1.00 | 40.32 | | O |
| ANISOU | 2487 | O | TYR | A | 320 | 5746 | 5359 | 4214 | −563 | 595 | −201 | O |
| ATOM | 2488 | N | ILE | A | 321 | 64.848 | 40.145 | 35.982 | 1.00 | 34.08 | | N |
| ANISOU | 2488 | N | ILE | A | 321 | 5039 | 4593 | 3317 | −492 | 545 | −280 | N |
| ATOM | 2489 | CA | ILE | A | 321 | 66.114 | 39.478 | 35.754 | 1.00 | 34.28 | | C |
| ANISOU | 2489 | CA | ILE | A | 321 | 5102 | 4695 | 3230 | −343 | 408 | −288 | C |
| ATOM | 2490 | CB | ILE | A | 321 | 67.217 | 40.187 | 36.584 | 1.00 | 37.27 | | C |
| ANISOU | 2490 | CB | ILE | A | 321 | 5430 | 5187 | 3545 | −277 | 317 | −363 | C |
| ATOM | 2491 | CG1 | ILE | A | 321 | 67.616 | 41.525 | 35.915 | 1.00 | 37.22 | | C |
| ANISOU | 2491 | CG1 | ILE | A | 321 | 5160 | 5279 | 3701 | −358 | 307 | −397 | C |
| ATOM | 2492 | CD1 | ILE | A | 321 | 68.186 | 42.532 | 36.854 | 1.00 | 40.17 | | C |
| ANISOU | 2492 | CD1 | ILE | A | 321 | 5494 | 5719 | 4052 | −394 | 285 | −485 | C |
| ATOM | 2493 | CG2 | ILE | A | 321 | 68.430 | 39.284 | 36.831 | 1.00 | 38.01 | | C |
| ANISOU | 2493 | CG2 | ILE | A | 321 | 5617 | 5377 | 3449 | −82 | 179 | −383 | C |
| ATOM | 2494 | C | ILE | A | 321 | 65.832 | 38.001 | 36.152 | 1.00 | 40.05 | | C |
| ANISOU | 2494 | C | ILE | A | 321 | 6122 | 5309 | 3788 | −282 | 429 | −254 | C |
| ATOM | 2495 | O | ILE | A | 321 | 65.939 | 37.104 | 35.312 | 1.00 | 39.66 | | O |
| ANISOU | 2495 | O | ILE | A | 321 | 6131 | 5239 | 3701 | −229 | 401 | −215 | O |
| ATOM | 2496 | N | ALA | A | 322 | 65.332 | 37.793 | 37.380 | 1.00 | 38.47 | | N |
| ANISOU | 2496 | N | ALA | A | 322 | 6118 | 5011 | 3488 | −309 | 501 | −266 | N |
| ATOM | 2497 | CA | ALA | A | 322 | 64.978 | 36.485 | 37.942 | 1.00 | 39.13 | | C |
| ANISOU | 2497 | CA | ALA | A | 322 | 6525 | 4949 | 3392 | −274 | 556 | −236 | C |
| ATOM | 2498 | CB | ALA | A | 322 | 64.623 | 36.627 | 39.414 | 1.00 | 39.88 | | C |
| ANISOU | 2498 | CB | ALA | A | 322 | 6797 | 4972 | 3382 | −291 | 623 | −262 | C |
| ATOM | 2499 | C | ALA | A | 322 | 63.846 | 35.783 | 37.206 | 1.00 | 42.86 | | C |
| ANISOU | 2499 | C | ALA | A | 322 | 7054 | 5312 | 3917 | −424 | 675 | −193 | C |
| ATOM | 2500 | O | ALA | A | 322 | 63.900 | 34.566 | 37.074 | 1.00 | 43.56 | | O |
| ANISOU | 2500 | O | ALA | A | 322 | 7389 | 5295 | 3867 | −381 | 690 | −163 | O |
| ATOM | 2501 | N | VAL | A | 323 | 62.795 | 36.529 | 36.807 | 1.00 | 37.78 | | N |
| ANISOU | 2501 | N | VAL | A | 323 | 6202 | 4698 | 3455 | −597 | 767 | −195 | N |
| ATOM | 2502 | CA | VAL | A | 323 | 61.635 | 36.005 | 36.072 | 1.00 | 36.73 | | C |
| ANISOU | 2502 | CA | VAL | A | 323 | 6058 | 4518 | 3381 | −763 | 879 | −175 | C |
| ATOM | 2503 | CB | VAL | A | 323 | 60.386 | 35.779 | 36.958 | 1.00 | 39.44 | | C |
| ANISOU | 2503 | CB | VAL | A | 323 | 6535 | 4770 | 3681 | −928 | 1049 | −189 | C |
| ATOM | 2504 | CG1 | VAL | A | 323 | 59.102 | 35.622 | 36.145 | 1.00 | 38.69 | | C |
| ANISOU | 2504 | CG1 | VAL | A | 323 | 6299 | 4712 | 3688 | −1127 | 1162 | −195 | C |
| ATOM | 2505 | CG2 | VAL | A | 323 | 60.603 | 34.551 | 37.801 | 1.00 | 39.38 | | C |
| ANISOU | 2505 | CG2 | VAL | A | 323 | 6914 | 4599 | 3451 | −894 | 1087 | −178 | C |
| ATOM | 2506 | C | VAL | A | 323 | 61.408 | 36.867 | 34.807 | 1.00 | 39.88 | | C |
| ANISOU | 2506 | C | VAL | A | 323 | 6125 | 5047 | 3980 | −806 | 852 | −169 | C |
| ATOM | 2507 | O | VAL | A | 323 | 60.631 | 37.842 | 34.779 | 1.00 | 37.80 | | O |
| ANISOU | 2507 | O | VAL | A | 323 | 5671 | 4844 | 3846 | −892 | 922 | −181 | O |
| ATOM | 2508 | N | PRO | A | 324 | 62.154 | 36.519 | 33.746 | 1.00 | 37.62 | | N |
| ANISOU | 2508 | N | PRO | A | 324 | 5785 | 4805 | 3705 | −718 | 749 | −148 | N |
| ATOM | 2509 | CA | PRO | A | 324 | 62.057 | 37.308 | 32.519 | 1.00 | 37.83 | | C |
| ANISOU | 2509 | CA | PRO | A | 324 | 5525 | 4949 | 3901 | −734 | 718 | −136 | C |
| ATOM | 2510 | CB | PRO | A | 324 | 63.245 | 36.838 | 31.671 | 1.00 | 39.41 | | C |
| ANISOU | 2510 | CB | PRO | A | 324 | 5726 | 5187 | 4061 | −594 | 588 | −117 | C |
| ATOM | 2511 | CG | PRO | A | 324 | 64.118 | 36.054 | 32.625 | 1.00 | 44.22 | | C |
| ANISOU | 2511 | CG | PRO | A | 324 | 6592 | 5728 | 4482 | −466 | 535 | −125 | C |
| ATOM | 2512 | CD | PRO | A | 324 | 63.148 | 35.441 | 33.601 | 1.00 | 39.55 | | C |
| ANISOU | 2512 | CD | PRO | A | 324 | 6231 | 5000 | 3797 | −576 | 658 | −132 | C |
| ATOM | 2513 | C | PRO | A | 324 | 60.715 | 37.112 | 31.868 | 1.00 | 42.72 | | C |
| ANISOU | 2513 | C | PRO | A | 324 | 6062 | 5581 | 4590 | −892 | 821 | −133 | C |
| ATOM | 2514 | O | PRO | A | 324 | 60.076 | 36.055 | 32.038 | 1.00 | 42.85 | | O |
| ANISOU | 2514 | O | PRO | A | 324 | 6255 | 5514 | 4512 | −998 | 896 | −142 | O |
| ATOM | 2515 | N | LYS | A | 325 | 60.274 | 38.116 | 31.118 | 1.00 | 38.41 | | N |
| ANISOU | 2515 | N | LYS | A | 325 | 5254 | 5144 | 4196 | −911 | 835 | −129 | N |
| ATOM | 2516 | CA | LYS | A | 325 | 58.937 | 38.066 | 30.475 | 1.00 | 37.52 | | C |
| ANISOU | 2516 | CA | LYS | A | 325 | 4994 | 5107 | 4156 | −1030 | 918 | −134 | C |
| ATOM | 2517 | CB | LYS | A | 325 | 58.541 | 39.453 | 29.965 | 1.00 | 38.16 | | C |
| ANISOU | 2517 | CB | LYS | A | 325 | 4817 | 5300 | 4382 | −975 | 923 | −121 | C |
| ATOM | 2518 | CG | LYS | A | 325 | 58.372 | 40.516 | 31.041 | 1.00 | 33.22 | | C |
| ANISOU | 2518 | CG | LYS | A | 325 | 4189 | 4654 | 3780 | −968 | 993 | −139 | C |
| ATOM | 2519 | CD | LYS | A | 325 | 58.152 | 41.896 | 30.473 | 1.00 | 36.46 | | C |
| ANISOU | 2519 | CD | LYS | A | 325 | 4388 | 5153 | 4313 | −907 | 1022 | −123 | C |
| ATOM | 2520 | CE | LYS | A | 325 | 57.684 | 42.900 | 31.502 | 1.00 | 38.91 | | C |
| ANISOU | 2520 | CE | LYS | A | 325 | 4706 | 5440 | 4636 | −904 | 1116 | −144 | C |
| ATOM | 2521 | NZ | LYS | A | 325 | 56.228 | 42.799 | 31.745 | 1.00 | 37.64 | | N |
| ANISOU | 2521 | NZ | LYS | A | 325 | 4475 | 5360 | 4467 | −983 | 1237 | −165 | N |
| ATOM | 2522 | C | LYS | A | 325 | 58.961 | 37.031 | 29.349 | 1.00 | 43.02 | | C |
| ANISOU | 2522 | C | LYS | A | 325 | 5718 | 5811 | 4818 | −1062 | 876 | −126 | C |
| ATOM | 2523 | O | LYS | A | 325 | 60.038 | 36.504 | 29.048 | 1.00 | 42.16 | | O |
| ANISOU | 2523 | O | LYS | A | 325 | 5702 | 5660 | 4658 | −955 | 777 | −105 | O |
| ATOM | 2524 | N | PRO | A | 326 | 57.814 | 36.704 | 28.720 | 1.00 | 41.54 | | N |
| ANISOU | 2524 | N | PRO | A | 326 | 5466 | 5682 | 4635 | −1211 | 952 | −153 | N |
| ATOM | 2525 | CA | PRO | A | 326 | 57.761 | 35.702 | 27.650 | 1.00 | 41.77 | | C |
| ANISOU | 2525 | CA | PRO | A | 326 | 5547 | 5708 | 4617 | −1256 | 914 | −157 | C |
| ATOM | 2526 | CB | PRO | A | 326 | 56.287 | 35.682 | 27.225 | 1.00 | 43.86 | | C |
| ANISOU | 2526 | CB | PRO | A | 326 | 5687 | 6084 | 4896 | −1454 | 1017 | −210 | C |
| ATOM | 2527 | CG | PRO | A | 326 | 55.729 | 36.986 | 27.739 | 1.00 | 48.73 | | C |
| ANISOU | 2527 | CG | PRO | A | 326 | 6089 | 6817 | 5608 | −1435 | 1075 | −214 | C |
| ATOM | 2528 | CD | PRO | A | 326 | 56.504 | 37.273 | 29.005 | 1.00 | 44.15 | | C |
| ANISOU | 2528 | CD | PRO | A | 326 | 5661 | 6110 | 5004 | −1345 | 1074 | −191 | C |
| ATOM | 2529 | C | PRO | A | 326 | 58.677 | 36.068 | 26.471 | 1.00 | 45.76 | | C |
| ANISOU | 2529 | C | PRO | A | 326 | 5908 | 6283 | 5195 | −1106 | 792 | −117 | C |
| ATOM | 2530 | O | PRO | A | 326 | 59.272 | 35.175 | 25.912 | 1.00 | 45.70 | | O |
| ANISOU | 2530 | O | PRO | A | 326 | 6030 | 6218 | 5117 | −1066 | 728 | −106 | O |
| ATOM | 2531 | N | ASN | A | 327 | 58.784 | 37.365 | 26.147 | 1.00 | 40.91 | | N |
| ANISOU | 2531 | N | ASN | A | 327 | 5061 | 5776 | 4708 | −1011 | 768 | −91 | N |
| ATOM | 2532 | CA | ASN | A | 327 | 59.675 | 37.913 | 25.087 | 1.00 | 39.73 | | C |
| ANISOU | 2532 | CA | ASN | A | 327 | 4782 | 5687 | 4628 | −875 | 672 | −50 | C |
| ATOM | 2533 | CB | ASN | A | 327 | 59.224 | 39.298 | 24.625 | 1.00 | 38.25 | | C |
| ANISOU | 2533 | CB | ASN | A | 327 | 4347 | 5624 | 4562 | −833 | 699 | −31 | C |
| ATOM | 2534 | CG | ASN | A | 327 | 59.469 | 40.367 | 25.662 | 1.00 | 60.89 | | C |
| ANISOU | 2534 | CG | ASN | A | 327 | 7174 | 8472 | 7490 | −779 | 741 | −22 | C |
| ATOM | 2535 | OD1 | ASN | A | 327 | 60.119 | 40.116 | 26.668 | 1.00 | 58.12 | | O |
| ANISOU | 2535 | OD1 | ASN | A | 327 | 6941 | 8045 | 7097 | −822 | 784 | −47 | O |
| ATOM | 2536 | ND2 | ASN | A | 327 | 58.954 | 41.560 | 25.424 | 1.00 | 53.90 | | N |
| ANISOU | 2536 | ND2 | ASN | A | 327 | 6138 | 7647 | 6693 | −684 | 738 | 12 | N |
| ATOM | 2537 | C | ASN | A | 327 | 61.151 | 37.901 | 25.524 | 1.00 | 44.19 | | C |
| ANISOU | 2537 | C | ASN | A | 327 | 5441 | 6184 | 5166 | −742 | 588 | −27 | C |
| ATOM | 2538 | O | ASN | A | 327 | 62.006 | 38.143 | 24.661 | 1.00 | 43.69 | | O |
| ANISOU | 2538 | O | ASN | A | 327 | 5270 | 6174 | 5156 | −639 | 520 | 2 | O |
| ATOM | 2539 | N | GLY | A | 328 | 61.442 | 37.668 | 26.809 | 1.00 | 42.53 | | N |
| ANISOU | 2539 | N | GLY | A | 328 | 5431 | 5872 | 4856 | −747 | 596 | −47 | N |
| ATOM | 2540 | CA | GLY | A | 328 | 62.825 | 37.603 | 27.317 | 1.00 | 42.84 | | C |
| ANISOU | 2540 | CA | GLY | A | 328 | 5569 | 5879 | 4831 | −618 | 512 | −44 | C |
| ATOM | 2541 | C | GLY | A | 328 | 63.276 | 38.907 | 27.935 | 1.00 | 47.38 | | C |
| ANISOU | 2541 | C | GLY | A | 328 | 6047 | 6487 | 5469 | −580 | 509 | −55 | C |
| ATOM | 2542 | O | GLY | A | 328 | 64.462 | 39.016 | 28.263 | 1.00 | 47.10 | | O |
| ANISOU | 2542 | O | GLY | A | 328 | 6024 | 6479 | 5394 | −480 | 428 | −65 | O |
| ATOM | 2543 | N | TYR | A | 329 | 62.359 | 39.856 | 28.108 | 1.00 | 44.48 | | N |
| ANISOU | 2543 | N | TYR | A | 329 | 5584 | 6129 | 5187 | −658 | 599 | −62 | N |
| ATOM | 2544 | CA | TYR | A | 329 | 62.755 | 41.159 | 28.693 | 1.00 | 45.26 | | C |
| ANISOU | 2544 | CA | TYR | A | 329 | 5617 | 6237 | 5344 | −639 | 615 | −79 | C |
| ATOM | 2545 | CB | TYR | A | 329 | 61.528 | 42.065 | 28.807 | 1.00 | 47.79 | | C |
| ANISOU | 2545 | CB | TYR | A | 329 | 5822 | 6574 | 5761 | −696 | 725 | −74 | C |
| ATOM | 2546 | CG | TYR | A | 329 | 61.767 | 43.396 | 29.471 | 1.00 | 51.72 | | C |
| ANISOU | 2546 | CG | TYR | A | 329 | 6285 | 7054 | 6312 | −678 | 761 | −91 | C |
| ATOM | 2547 | CD1 | TYR | A | 329 | 62.561 | 44.356 | 28.876 | 1.00 | 54.28 | | C |
| ANISOU | 2547 | CD1 | TYR | A | 329 | 6509 | 7407 | 6710 | −632 | 734 | −79 | C |
| ATOM | 2548 | CE1 | TYR | A | 329 | 62.777 | 45.585 | 29.475 | 1.00 | 56.06 | | C |
| ANISOU | 2548 | CE1 | TYR | A | 329 | 6734 | 7593 | 6974 | −640 | 784 | −104 | C |
| ATOM | 2549 | CZ | TYR | A | 329 | 62.189 | 45.871 | 30.691 | 1.00 | 65.02 | | C |
| ANISOU | 2549 | CZ | TYR | A | 329 | 7960 | 8668 | 8077 | −677 | 853 | −139 | C |
| ATOM | 2550 | OH | TYR | A | 329 | 62.404 | 47.081 | 31.280 | 1.00 | 68.42 | | O |
| ANISOU | 2550 | OH | TYR | A | 329 | 8415 | 9046 | 8536 | −691 | 909 | −171 | O |
| ATOM | 2551 | CE2 | TYR | A | 329 | 61.387 | 44.927 | 31.300 | 1.00 | 54.89 | | C |
| ANISOU | 2551 | CE2 | TYR | A | 329 | 6762 | 7369 | 6724 | −712 | 877 | −147 | C |
| ATOM | 2552 | CD2 | TYR | A | 329 | 61.182 | 43.704 | 30.686 | 1.00 | 53.33 | | C |
| ANISOU | 2552 | CD2 | TYR | A | 329 | 6573 | 7203 | 6487 | −721 | 836 | −124 | C |
| ATOM | 2553 | C | TYR | A | 329 | 63.357 | 40.907 | 30.076 | 1.00 | 47.60 | | C |
| ANISOU | 2553 | C | TYR | A | 329 | 6079 | 6475 | 5533 | −620 | 595 | −118 | C |
| ATOM | 2554 | O | TYR | A | 329 | 62.751 | 40.184 | 30.878 | 1.00 | 47.05 | | O |
| ANISOU | 2554 | O | TYR | A | 329 | 6161 | 6333 | 5382 | −671 | 650 | −131 | O |
| ATOM | 2555 | N | GLN | A | 330 | 64.529 | 41.490 | 30.328 | 1.00 | 41.73 | | N |
| ANISOU | 2555 | N | GLN | A | 330 | 5301 | 5778 | 4777 | −551 | 516 | −143 | N |
| ATOM | 2556 | CA | GLN | A | 330 | 65.210 | 41.346 | 31.636 | 1.00 | 40.04 | | C |
| ANISOU | 2556 | CA | GLN | A | 330 | 5199 | 5557 | 4456 | −512 | 472 | −193 | C |
| ATOM | 2557 | CB | GLN | A | 330 | 66.501 | 40.554 | 31.451 | 1.00 | 40.79 | | C |
| ANISOU | 2557 | CB | GLN | A | 330 | 5337 | 5725 | 4435 | −385 | 344 | −206 | C |
| ATOM | 2558 | CG | GLN | A | 330 | 66.452 | 39.145 | 32.011 | 1.00 | 48.36 | | C |
| ANISOU | 2558 | CG | GLN | A | 330 | 6524 | 6608 | 5241 | −317 | 326 | −185 | C |
| ATOM | 2559 | CD | GLN | A | 330 | 67.765 | 38.449 | 31.770 | 1.00 | 72.96 | | C |
| ANISOU | 2559 | CD | GLN | A | 330 | 9678 | 9810 | 8233 | −147 | 199 | −192 | C |
| ATOM | 2560 | OE1 | GLN | A | 330 | 68.579 | 38.892 | 30.968 | 1.00 | 71.49 | | O |
| ANISOU | 2560 | OE1 | GLN | A | 330 | 9332 | 9728 | 8103 | −102 | 140 | −184 | O |
| ATOM | 2561 | NE2 | GLN | A | 330 | 67.984 | 37.351 | 32.469 | 1.00 | 64.68 | | N |
| ANISOU | 2561 | NE2 | GLN | A | 330 | 8851 | 8725 | 7001 | −34 | 161 | −205 | N |
| ATOM | 2562 | C | GLN | A | 330 | 65.568 | 42.740 | 32.144 | 1.00 | 44.13 | | C |
| ANISOU | 2562 | C | GLN | A | 330 | 5611 | 6102 | 5053 | −557 | 499 | −234 | C |
| ATOM | 2563 | O | GLN | A | 330 | 66.386 | 43.406 | 31.497 | 1.00 | 42.82 | | O |
| ANISOU | 2563 | O | GLN | A | 330 | 5295 | 5992 | 4981 | −566 | 490 | −230 | O |
| ATOM | 2564 | N | SER | A | 331 | 64.981 | 43.144 | 33.269 | 1.00 | 42.40 | | N |
| ANISOU | 2564 | N | SER | A | 331 | 5494 | 5829 | 4788 | −593 | 547 | −274 | N |
| ATOM | 2565 | CA | SER | A | 331 | 65.260 | 44.465 | 33.885 | 1.00 | 41.72 | | C |
| ANISOU | 2565 | CA | SER | A | 331 | 5362 | 5737 | 4752 | −649 | 590 | −324 | C |
| ATOM | 2566 | CB | SER | A | 331 | 64.621 | 45.570 | 33.090 | 1.00 | 43.57 | | C |
| ANISOU | 2566 | CB | SER | A | 331 | 5474 | 5943 | 5136 | −697 | 686 | −288 | C |
| ATOM | 2567 | OG | SER | A | 331 | 64.843 | 46.830 | 33.695 | 1.00 | 50.53 | | O |
| ANISOU | 2567 | OG | SER | A | 331 | 6347 | 6793 | 6057 | −750 | 740 | −336 | O |
| ATOM | 2568 | C | SER | A | 331 | 64.774 | 44.447 | 35.337 | 1.00 | 44.60 | | C |
| ANISOU | 2568 | C | SER | A | 331 | 5880 | 6024 | 5042 | −676 | 652 | −355 | C |
| ATOM | 2569 | O | SER | A | 331 | 63.991 | 43.548 | 35.681 | 1.00 | 43.35 | | O |
| ANISOU | 2569 | O | SER | A | 331 | 5828 | 5805 | 4839 | −682 | 705 | −323 | O |
| ATOM | 2570 | N | LEU | A | 332 | 65.235 | 45.409 | 36.141 | 1.00 | 40.82 | | N |
| ANISOU | 2570 | N | LEU | A | 332 | 5418 | 5548 | 4545 | −709 | 655 | −425 | N |
| ATOM | 2571 | CA | LEU | A | 332 | 64.810 | 45.544 | 37.560 | 1.00 | 40.62 | | C |
| ANISOU | 2571 | CA | LEU | A | 332 | 5534 | 5451 | 4450 | −734 | 718 | −463 | C |
| ATOM | 2572 | CB | LEU | A | 332 | 66.055 | 45.618 | 38.449 | 1.00 | 40.82 | | C |
| ANISOU | 2572 | CB | LEU | A | 332 | 5622 | 5542 | 4346 | −709 | 618 | −552 | C |
| ATOM | 2573 | CG | LEU | A | 332 | 66.327 | 44.380 | 39.303 | 1.00 | 45.50 | | C |
| ANISOU | 2573 | CG | LEU | A | 332 | 6376 | 6150 | 4763 | −607 | 543 | −551 | C |
| ATOM | 2574 | CD2 | LEU | A | 332 | 65.027 | 43.772 | 39.805 | 1.00 | 48.76 | | C |
| ANISOU | 2574 | CD2 | LEU | A | 332 | 6968 | 6437 | 5121 | −627 | 653 | −525 | C |
| ATOM | 2575 | CD1 | LEU | A | 332 | 67.242 | 44.718 | 40.470 | 1.00 | 45.63 | | C |
| ANISOU | 2575 | CD1 | LEU | A | 332 | 6402 | 6290 | 4643 | −551 | 418 | −645 | C |
| ATOM | 2576 | C | LEU | A | 332 | 63.985 | 46.830 | 37.658 | 1.00 | 44.49 | | C |
| ANISOU | 2576 | C | LEU | A | 332 | 5983 | 5871 | 5051 | −801 | 846 | −466 | C |
| ATOM | 2577 | O | LEU | A | 332 | 64.483 | 47.877 | 37.201 | 1.00 | 44.41 | | O |
| ANISOU | 2577 | O | LEU | A | 332 | 5878 | 5875 | 5120 | −835 | 850 | −485 | O |
| ATOM | 2578 | N | HIS | A | 333 | 62.777 | 46.759 | 38.225 | 1.00 | 40.09 | | N |
| ANISOU | 2578 | N | HIS | A | 333 | 5502 | 5239 | 4492 | −815 | 962 | −444 | N |
| ATOM | 2579 | CA | HIS | A | 333 | 61.939 | 47.950 | 38.275 | 1.00 | 39.59 | | C |
| ANISOU | 2579 | CA | HIS | A | 333 | 5412 | 5116 | 4515 | −839 | 1092 | −442 | C |
| ATOM | 2580 | CB | HIS | A | 333 | 60.600 | 47.738 | 37.564 | 1.00 | 40.33 | | C |
| ANISOU | 2580 | CB | HIS | A | 333 | 5420 | 5222 | 4683 | −820 | 1186 | −369 | C |
| ATOM | 2581 | CG | HIS | A | 333 | 60.657 | 47.716 | 36.068 | 1.00 | 43.79 | | C |
| ANISOU | 2581 | CG | HIS | A | 333 | 5703 | 5717 | 5217 | −792 | 1150 | −315 | C |
| ATOM | 2582 | ND1 | HIS | A | 333 | 59.501 | 47.807 | 35.307 | 1.00 | 45.35 | | N |
| ANISOU | 2582 | ND1 | HIS | A | 333 | 5793 | 5954 | 5485 | −761 | 1230 | −262 | N |
| ATOM | 2583 | CE1 | HIS | A | 333 | 59.897 | 47.745 | 34.049 | 1.00 | 44.73 | | C |
| ANISOU | 2583 | CE1 | HIS | A | 333 | 5604 | 5923 | 5469 | −733 | 1168 | −224 | C |
| ATOM | 2584 | NE2 | HIS | A | 333 | 61.222 | 47.608 | 33.954 | 1.00 | 45.07 | | N |
| ANISOU | 2584 | NE2 | HIS | A | 333 | 5672 | 5963 | 5489 | −749 | 1061 | −249 | N |
| ATOM | 2585 | CD2 | HIS | A | 333 | 61.719 | 47.588 | 35.234 | 1.00 | 45.39 | | C |
| ANISOU | 2585 | CD2 | HIS | A | 333 | 5841 | 5961 | 5442 | −783 | 1044 | −311 | C |
| ATOM | 2586 | C | HIS | A | 333 | 61.641 | 48.234 | 39.698 | 1.00 | 45.23 | | C |
| ANISOU | 2586 | C | HIS | A | 333 | 6272 | 5766 | 5147 | −858 | 1158 | −494 | C |
| ATOM | 2587 | O | HIS | A | 333 | 61.515 | 47.304 | 40.499 | 1.00 | 45.83 | | O |
| ANISOU | 2587 | O | HIS | A | 333 | 6461 | 5833 | 5119 | −853 | 1153 | −496 | O |
| ATOM | 2588 | N | THR | A | 334 | 61.490 | 49.522 | 40.019 | 1.00 | 41.66 | | N |
| ANISOU | 2588 | N | THR | A | 334 | 5843 | 5253 | 4732 | −876 | 1233 | −533 | N |
| ATOM | 2589 | CA | THR | A | 334 | 61.128 | 50.002 | 41.348 | 1.00 | 40.71 | | C |
| ANISOU | 2589 | CA | THR | A | 334 | 5868 | 5062 | 4538 | −890 | 1312 | −587 | C |
| ATOM | 2590 | CB | THR | A | 334 | 62.271 | 49.856 | 42.384 | 1.00 | 51.87 | | C |
| ANISOU | 2590 | CB | THR | A | 334 | 7392 | 6493 | 5825 | −923 | 1206 | −678 | C |
| ATOM | 2591 | OG1 | THR | A | 334 | 61.830 | 50.360 | 43.658 | 1.00 | 54.97 | | O |
| ANISOU | 2591 | OG1 | THR | A | 334 | 7937 | 6811 | 6138 | −932 | 1291 | −728 | O |
| ATOM | 2592 | CG2 | THR | A | 334 | 63.576 | 50.515 | 41.937 | 1.00 | 50.27 | | C |
| ANISOU | 2592 | CG2 | THR | A | 334 | 7122 | 6333 | 5645 | −980 | 1118 | −745 | C |
| ATOM | 2593 | C | THR | A | 334 | 60.555 | 51.404 | 41.273 | 1.00 | 42.83 | | C |
| ANISOU | 2593 | C | THR | A | 334 | 6147 | 5247 | 4877 | −879 | 1444 | −593 | C |
| ATOM | 2594 | O | THR | A | 334 | 61.168 | 52.290 | 40.686 | 1.00 | 42.13 | | O |
| ANISOU | 2594 | O | THR | A | 334 | 6028 | 5130 | 4849 | −902 | 1438 | −610 | O |
| ATOM | 2595 | N | THR | A | 335 | 59.365 | 51.593 | 41.846 | 1.00 | 39.20 | | N |
| ANISOU | 2595 | N | THR | A | 335 | 5742 | 4749 | 4401 | −838 | 1574 | −577 | N |
| ATOM | 2596 | CA | THR | A | 335 | 58.760 | 52.905 | 41.931 | 1.00 | 38.81 | | C |
| ANISOU | 2596 | CA | THR | A | 335 | 5741 | 4616 | 4390 | −790 | 1710 | −584 | C |
| ATOM | 2597 | CB | THR | A | 335 | 57.301 | 52.909 | 41.572 | 1.00 | 45.99 | | C |
| ANISOU | 2597 | CB | THR | A | 335 | 6564 | 5570 | 5341 | −693 | 1835 | −516 | C |
| ATOM | 2598 | OG1 | THR | A | 335 | 57.104 | 52.089 | 40.420 | 1.00 | 49.81 | | O |
| ANISOU | 2598 | OG1 | THR | A | 335 | 6874 | 6165 | 5887 | −682 | 1776 | −449 | O |
| ATOM | 2599 | CG2 | THR | A | 335 | 56.801 | 54.303 | 41.298 | 1.00 | 43.74 | | C |
| ANISOU | 2599 | CG2 | THR | A | 335 | 6314 | 5207 | 5098 | −593 | 1958 | −506 | C |
| ATOM | 2600 | C | THR | A | 335 | 59.069 | 53.438 | 43.317 | 1.00 | 43.95 | | C |
| ANISOU | 2600 | C | THR | A | 335 | 6577 | 5180 | 4942 | −832 | 1739 | −673 | C |
| ATOM | 2601 | O | THR | A | 335 | 58.851 | 52.751 | 44.335 | 1.00 | 44.01 | | O |
| ANISOU | 2601 | O | THR | A | 335 | 6664 | 5202 | 4853 | −844 | 1738 | −696 | O |
| ATOM | 2602 | N | VAL | A | 336 | 59.673 | 54.644 | 43.324 | 1.00 | 39.68 | | N |
| ANISOU | 2602 | N | VAL | A | 336 | 6119 | 4543 | 4414 | −867 | 1764 | −730 | N |
| ATOM | 2603 | CA | VAL | A | 336 | 60.165 | 55.396 | 44.481 | 1.00 | 38.00 | | C |
| ANISOU | 2603 | CA | VAL | A | 336 | 6090 | 4237 | 4112 | −934 | 1789 | −837 | C |
| ATOM | 2604 | CB | VAL | A | 336 | 61.718 | 55.538 | 44.413 | 1.00 | 39.87 | | C |
| ANISOU | 2604 | CB | VAL | A | 336 | 6324 | 4502 | 4323 | −1071 | 1655 | −930 | C |
| ATOM | 2605 | CG1 | VAL | A | 336 | 62.389 | 54.173 | 44.351 | 1.00 | 39.33 | | C |
| ANISOU | 2605 | CG1 | VAL | A | 336 | 6146 | 4587 | 4210 | −1084 | 1485 | −925 | C |
| ATOM | 2606 | CG2 | VAL | A | 336 | 62.158 | 56.386 | 43.229 | 1.00 | 38.97 | | C |
| ANISOU | 2606 | CG2 | VAL | A | 336 | 6158 | 4340 | 4308 | −1113 | 1680 | −915 | C |
| ATOM | 2607 | C | VAL | A | 336 | 59.428 | 56.761 | 44.638 | 1.00 | 43.35 | | C |
| ANISOU | 2607 | C | VAL | A | 336 | 6899 | 4766 | 4805 | −869 | 1967 | −842 | C |
| ATOM | 2608 | O | VAL | A | 336 | 58.608 | 57.130 | 43.792 | 1.00 | 43.60 | | O |
| ANISOU | 2608 | O | VAL | A | 336 | 6868 | 4786 | 4912 | −753 | 2060 | −758 | O |
| ATOM | 2609 | N | ILE | A | 337 | 59.690 | 57.474 | 45.735 | 1.00 | 39.83 | | N |
| ANISOU | 2609 | N | ILE | A | 337 | 6646 | 4217 | 4271 | −926 | 2013 | −941 | N |
| ATOM | 2610 | CA | ILE | A | 337 | 59.119 | 58.780 | 46.008 | 1.00 | 39.70 | | C |
| ANISOU | 2610 | CA | ILE | A | 337 | 6808 | 4035 | 4240 | −866 | 2182 | −961 | C |
| ATOM | 2611 | CB | ILE | A | 337 | 58.426 | 58.839 | 47.382 | 1.00 | 41.76 | | C |
| ANISOU | 2611 | CB | ILE | A | 337 | 7217 | 4257 | 4393 | −817 | 2266 | −1004 | C |
| ATOM | 2612 | CG1 | ILE | A | 337 | 57.409 | 57.729 | 47.516 | 1.00 | 42.09 | | C |
| ANISOU | 2612 | CG1 | ILE | A | 337 | 7127 | 4432 | 4433 | −719 | 2276 | −922 | C |
| ATOM | 2613 | CD1 | ILE | A | 337 | 56.939 | 57.499 | 48.871 | 1.00 | 46.01 | | C |
| ANISOU | 2613 | CD1 | ILE | A | 337 | 7747 | 4919 | 4814 | −708 | 2328 | −967 | C |
| ATOM | 2614 | CG2 | ILE | A | 337 | 57.765 | 60.181 | 47.600 | 1.00 | 41.62 | | C |
| ANISOU | 2614 | CG2 | ILE | A | 337 | 7396 | 4065 | 4352 | −722 | 2452 | −1018 | C |
| ATOM | 2615 | C | ILE | A | 337 | 60.303 | 59.702 | 45.941 | 1.00 | 49.26 | | C |
| ANISOU | 2615 | C | ILE | A | 337 | 8137 | 5142 | 5436 | −1026 | 2158 | −1063 | C |
| ATOM | 2616 | O | ILE | A | 337 | 61.084 | 59.779 | 46.894 | 1.00 | 49.97 | | O |
| ANISOU | 2616 | O | ILE | A | 337 | 8319 | 5233 | 5433 | −1160 | 2093 | −1182 | O |
| ATOM | 2617 | N | ALA | A | 338 | 60.480 | 60.331 | 44.779 | 1.00 | 49.01 | | N |
| ANISOU | 2617 | N | ALA | A | 338 | 8091 | 5042 | 5488 | −1024 | 2204 | −1021 | N |
| ATOM | 2618 | CA | ALA | A | 338 | 61.550 | 61.277 | 44.489 | 1.00 | 50.24 | | C |
| ANISOU | 2618 | CA | ALA | A | 338 | 8360 | 5090 | 5639 | −1201 | 2212 | −1112 | C |
| ATOM | 2619 | CB | ALA | A | 338 | 62.270 | 60.839 | 43.226 | 1.00 | 50.85 | | C |
| ANISOU | 2619 | CB | ALA | A | 338 | 8241 | 5274 | 5806 | −1264 | 2108 | −1067 | C |
| ATOM | 2620 | C | ALA | A | 338 | 60.945 | 62.674 | 44.296 | 1.00 | 58.60 | | C |
| ANISOU | 2620 | C | ALA | A | 338 | 9661 | 5911 | 6693 | −1121 | 2419 | −1095 | C |
| ATOM | 2621 | O | ALA | A | 338 | 59.743 | 62.852 | 44.492 | 1.00 | 58.31 | | O |
| ANISOU | 2621 | O | ALA | A | 338 | 9679 | 5828 | 6647 | −912 | 2534 | −1021 | O |
| ATOM | 2622 | N | LEU | A | 339 | 61.787 | 63.665 | 43.956 | 1.00 | 58.71 | | N |
| ANISOU | 2622 | N | LEU | A | 339 | 9831 | 5778 | 6697 | −1285 | 2476 | −1170 | N |
| ATOM | 2623 | CA | LEU | A | 339 | 61.422 | 65.053 | 43.627 | 1.00 | 60.19 | | C |
| ANISOU | 2623 | CA | LEU | A | 339 | 10302 | 5701 | 6865 | −1226 | 2683 | −1155 | C |
| ATOM | 2624 | CB | LEU | A | 339 | 61.043 | 65.129 | 42.142 | 1.00 | 60.23 | | C |
| ANISOU | 2624 | CB | LEU | A | 339 | 10220 | 5698 | 6965 | −1077 | 2726 | −1011 | C |
| ATOM | 2625 | CG | LEU | A | 339 | 62.179 | 64.788 | 41.204 | 1.00 | 65.93 | | C |
| ANISOU | 2625 | CG | LEU | A | 339 | 10778 | 6518 | 7755 | −1268 | 2612 | −1025 | C |
| ATOM | 2626 | CD1 | LEU | A | 339 | 61.669 | 64.191 | 39.896 | 1.00 | 66.65 | | C |
| ANISOU | 2626 | CD1 | LEU | A | 339 | 10670 | 6711 | 7944 | −1084 | 2582 | −869 | C |
| ATOM | 2627 | CD2 | LEU | A | 339 | 63.078 | 65.978 | 40.983 | 1.00 | 69.82 | | C |
| ANISOU | 2627 | CD2 | LEU | A | 339 | 11514 | 6807 | 8207 | −1497 | 2719 | −1126 | C |
| ATOM | 2628 | C | LEU | A | 339 | 60.317 | 65.692 | 44.508 | 1.00 | 67.04 | | C |
| ANISOU | 2628 | C | LEU | A | 339 | 11396 | 6425 | 7653 | −1036 | 2840 | −1147 | C |
| ATOM | 2629 | O | LEU | A | 339 | 59.410 | 66.372 | 44.003 | 1.00 | 66.57 | | O |
| ANISOU | 2629 | O | LEU | A | 339 | 11450 | 6247 | 7597 | −801 | 2990 | −1047 | O |
| ATOM | 2630 | N | GLU | A | 340 | 60.439 | 65.500 | 45.828 | 1.00 | 65.66 | | N |
| ANISOU | 2630 | N | GLU | A | 340 | 11288 | 6270 | 7389 | −1127 | 2803 | −1258 | N |
| ATOM | 2631 | CA | GLU | A | 340 | 59.523 | 66.026 | 46.849 | 1.00 | 66.64 | | C |
| ANISOU | 2631 | CA | GLU | A | 340 | 11627 | 6274 | 7419 | −983 | 2938 | −1277 | C |
| ATOM | 2632 | CB | GLU | A | 340 | 59.530 | 67.588 | 46.938 | 1.00 | 68.52 | | C |
| ANISOU | 2632 | CB | GLU | A | 340 | 12260 | 6193 | 7580 | −997 | 3145 | −1335 | C |
| ATOM | 2633 | CG | GLU | A | 340 | 60.860 | 68.216 | 47.333 | 1.00 | 83.41 | | C |
| ANISOU | 2633 | CG | GLU | A | 340 | 14327 | 7964 | 9402 | −1343 | 3128 | −1514 | C |
| ATOM | 2634 | CD | GLU | A | 340 | 61.619 | 68.852 | 46.180 | 1.00 | 112.37 | | C |
| ANISOU | 2634 | CD | GLU | A | 340 | 18062 | 11515 | 13120 | −1501 | 3173 | −1518 | C |
| ATOM | 2635 | OE1 | GLU | A | 340 | 61.541 | 70.094 | 46.036 | 1.00 | 107.53 | | O |
| ANISOU | 2635 | OE1 | GLU | A | 340 | 17799 | 10610 | 12447 | −1509 | 3370 | −1541 | O |
| ATOM | 2636 | OE2 | GLU | A | 340 | 62.286 | 68.112 | 45.420 | 1.00 | 107.70 | | O |
| ANISOU | 2636 | OE2 | GLU | A | 340 | 17191 | 11113 | 12615 | −1614 | 3023 | −1498 | O |
| ATOM | 2637 | C | GLU | A | 340 | 58.100 | 65.463 | 46.691 | 1.00 | 69.14 | | C |
| ANISOU | 2637 | C | GLU | A | 340 | 11795 | 6708 | 7769 | −675 | 2982 | −1134 | C |
| ATOM | 2638 | O | GLU | A | 340 | 57.161 | 66.214 | 46.403 | 1.00 | 69.92 | | O |
| ANISOU | 2638 | O | GLU | A | 340 | 12034 | 6686 | 7846 | −446 | 3149 | −1065 | O |
| ATOM | 2639 | N | GLY | A | 341 | 57.965 | 64.149 | 46.870 | 1.00 | 62.17 | | N |
| ANISOU | 2639 | N | GLY | A | 341 | 10636 | 6063 | 6922 | −674 | 2837 | −1099 | N |
| ATOM | 2640 | CA | GLY | A | 341 | 56.673 | 63.473 | 46.801 | 1.00 | 60.32 | | C |
| ANISOU | 2640 | CA | GLY | A | 341 | 10232 | 5977 | 6711 | −444 | 2871 | −988 | C |
| ATOM | 2641 | C | GLY | A | 341 | 56.140 | 63.169 | 45.414 | 1.00 | 59.99 | | C |
| ANISOU | 2641 | C | GLY | A | 341 | 9972 | 6048 | 6774 | −297 | 2861 | −855 | C |
| ATOM | 2642 | O | GLY | A | 341 | 54.948 | 63.353 | 45.153 | 1.00 | 59.51 | | O |
| ANISOU | 2642 | O | GLY | A | 341 | 9866 | 6034 | 6712 | −57 | 2970 | −772 | O |
| ATOM | 2643 | N | LEU | A | 342 | 56.996 | 62.647 | 44.537 | 1.00 | 53.58 | | N |
| ANISOU | 2643 | N | LEU | A | 342 | 9005 | 5308 | 6043 | −431 | 2724 | −840 | N |
| ATOM | 2644 | CA | LEU | A | 342 | 56.600 | 62.301 | 43.181 | 1.00 | 52.51 | | C |
| ANISOU | 2644 | CA | LEU | A | 342 | 8664 | 5285 | 6003 | −310 | 2698 | −721 | C |
| ATOM | 2645 | CB | LEU | A | 342 | 57.318 | 63.206 | 42.171 | 1.00 | 52.38 | | C |
| ANISOU | 2645 | CB | LEU | A | 342 | 8761 | 5117 | 6024 | −349 | 2735 | −709 | C |
| ATOM | 2646 | CG | LEU | A | 342 | 56.770 | 64.592 | 41.980 | 1.00 | 57.30 | | C |
| ANISOU | 2646 | CG | LEU | A | 342 | 9654 | 5523 | 6594 | −179 | 2932 | −685 | C |
| ATOM | 2647 | CD1 | LEU | A | 342 | 57.440 | 65.265 | 40.802 | 1.00 | 57.45 | | C |
| ANISOU | 2647 | CD1 | LEU | A | 342 | 9776 | 5403 | 6650 | −238 | 2963 | −661 | C |
| ATOM | 2648 | CD2 | LEU | A | 342 | 55.255 | 64.566 | 41.810 | 1.00 | 60.49 | | C |
| ANISOU | 2648 | CD2 | LEU | A | 342 | 9969 | 6031 | 6986 | 147 | 3026 | −580 | C |
| ATOM | 2649 | C | LEU | A | 342 | 56.917 | 60.852 | 42.838 | 1.00 | 55.74 | | C |
| ANISOU | 2649 | C | LEU | A | 342 | 8793 | 5910 | 6474 | −406 | 2519 | −694 | C |
| ATOM | 2650 | O | LEU | A | 342 | 58.086 | 60.460 | 42.962 | 1.00 | 57.39 | | O |
| ANISOU | 2650 | O | LEU | A | 342 | 8980 | 6136 | 6688 | −605 | 2390 | −759 | O |
| ATOM | 2651 | N | PRO | A | 343 | 55.940 | 60.049 | 42.331 | 1.00 | 48.00 | | N |
| ANISOU | 2651 | N | PRO | A | 343 | 7595 | 5106 | 5535 | −267 | 2510 | −603 | N |
| ATOM | 2652 | CA | PRO | A | 343 | 56.272 | 58.688 | 41.903 | 1.00 | 45.26 | | C |
| ANISOU | 2652 | CA | PRO | A | 343 | 7017 | 4938 | 5242 | −360 | 2353 | −575 | C |
| ATOM | 2653 | CB | PRO | A | 343 | 54.952 | 58.143 | 41.360 | 1.00 | 46.87 | | C |
| ANISOU | 2653 | CB | PRO | A | 343 | 7025 | 5310 | 5474 | −193 | 2399 | −487 | C |
| ATOM | 2654 | CG | PRO | A | 343 | 53.919 | 58.961 | 41.974 | 1.00 | 52.79 | | C |
| ANISOU | 2654 | CG | PRO | A | 343 | 7886 | 6013 | 6161 | −30 | 2564 | −491 | C |
| ATOM | 2655 | CD | PRO | A | 343 | 54.514 | 60.337 | 42.093 | 1.00 | 49.34 | | C |
| ANISOU | 2655 | CD | PRO | A | 343 | 7715 | 5338 | 5694 | −22 | 2643 | −531 | C |
| ATOM | 2656 | C | PRO | A | 343 | 57.296 | 58.764 | 40.795 | 1.00 | 45.30 | | C |
| ANISOU | 2656 | C | PRO | A | 343 | 6969 | 4924 | 5318 | −441 | 2263 | −557 | C |
| ATOM | 2657 | O | PRO | A | 343 | 57.190 | 59.582 | 39.887 | 1.00 | 44.48 | | O |
| ANISOU | 2657 | O | PRO | A | 343 | 6893 | 4755 | 5253 | −340 | 2334 | −504 | O |
| ATOM | 2658 | N | LEU | A | 344 | 58.335 | 57.981 | 40.936 | 1.00 | 40.57 | | N |
| ANISOU | 2658 | N | LEU | A | 344 | 6319 | 4377 | 4720 | −613 | 2118 | −608 | N |
| ATOM | 2659 | CA | LEU | A | 344 | 59.383 | 57.888 | 39.950 | 1.00 | 39.85 | | C |
| ANISOU | 2659 | CA | LEU | A | 344 | 6147 | 4307 | 4687 | −707 | 2019 | −601 | C |
| ATOM | 2660 | CB | LEU | A | 344 | 60.640 | 58.627 | 40.418 | 1.00 | 39.49 | | C |
| ANISOU | 2660 | CB | LEU | A | 344 | 6247 | 4156 | 4599 | −890 | 1995 | −715 | C |
| ATOM | 2661 | CG | LEU | A | 344 | 61.765 | 58.694 | 39.397 | 1.00 | 43.87 | | C |
| ANISOU | 2661 | CG | LEU | A | 344 | 6718 | 4743 | 5208 | −1006 | 1913 | −722 | C |
| ATOM | 2662 | CD1 | LEU | A | 344 | 61.643 | 59.926 | 38.524 | 1.00 | 44.34 | | C |
| ANISOU | 2662 | CD1 | LEU | A | 344 | 6888 | 4649 | 5309 | −964 | 2047 | −679 | C |
| ATOM | 2663 | CD2 | LEU | A | 344 | 63.088 | 58.711 | 40.080 | 1.00 | 45.48 | | C |
| ANISOU | 2663 | CD2 | LEU | A | 344 | 6963 | 4968 | 5350 | −1215 | 1825 | −857 | C |
| ATOM | 2664 | C | LEU | A | 344 | 59.651 | 56.407 | 39.750 | 1.00 | 44.89 | | C |
| ANISOU | 2664 | C | LEU | A | 344 | 6590 | 5125 | 5343 | −746 | 1865 | −575 | C |
| ATOM | 2665 | O | LEU | A | 344 | 59.916 | 55.701 | 40.721 | 1.00 | 44.98 | | O |
| ANISOU | 2665 | O | LEU | A | 344 | 6619 | 5185 | 5286 | −810 | 1797 | −626 | O |
| ATOM | 2666 | N | GLU | A | 345 | 59.507 | 55.922 | 38.509 | 1.00 | 41.63 | | N |
| ANISOU | 2666 | N | GLU | A | 345 | 6012 | 4800 | 5004 | −693 | 1819 | −493 | N |
| ATOM | 2667 | CA | GLU | A | 345 | 59.779 | 54.533 | 38.158 | 1.00 | 40.82 | | C |
| ANISOU | 2667 | CA | GLU | A | 345 | 5752 | 4846 | 4913 | −728 | 1680 | −467 | C |
| ATOM | 2668 | CB | GLU | A | 345 | 58.840 | 54.072 | 37.024 | 1.00 | 42.52 | | C |
| ANISOU | 2668 | CB | GLU | A | 345 | 5802 | 5163 | 5193 | −614 | 1694 | −368 | C |
| ATOM | 2669 | CG | GLU | A | 345 | 59.121 | 52.678 | 36.464 | 1.00 | 62.60 | | C |
| ANISOU | 2669 | CG | GLU | A | 345 | 8206 | 7835 | 7744 | −656 | 1562 | −340 | C |
| ATOM | 2670 | CD | GLU | A | 345 | 58.237 | 51.516 | 36.895 | 1.00 | 102.82 | | C |
| ANISOU | 2670 | CD | GLU | A | 345 | 13249 | 13019 | 12798 | −654 | 1564 | −325 | C |
| ATOM | 2671 | OE1 | GLU | A | 345 | 57.323 | 51.725 | 37.726 | 1.00 | 116.84 | | O |
| ANISOU | 2671 | OE1 | GLU | A | 345 | 15067 | 14787 | 14541 | −616 | 1673 | −334 | O |
| ATOM | 2672 | OE2 | GLU | A | 345 | 58.461 | 50.388 | 36.397 | 1.00 | 97.86 | | O |
| ANISOU | 2672 | OE2 | GLU | A | 345 | 12551 | 12468 | 12163 | −694 | 1467 | −306 | O |
| ATOM | 2673 | C | GLU | A | 345 | 61.262 | 54.477 | 37.754 | 1.00 | 42.40 | | C |
| ANISOU | 2673 | C | GLU | A | 345 | 5927 | 5062 | 5120 | −847 | 1561 | −512 | C |
| ATOM | 2674 | O | GLU | A | 345 | 61.724 | 55.315 | 36.973 | 1.00 | 41.74 | | O |
| ANISOU | 2674 | O | GLU | A | 345 | 5856 | 4919 | 5084 | −870 | 1594 | −507 | O |
| ATOM | 2675 | N | VAL | A | 346 | 62.023 | 53.558 | 38.363 | 1.00 | 37.47 | | N |
| ANISOU | 2675 | N | VAL | A | 346 | 5285 | 4520 | 4432 | −920 | 1435 | −563 | N |
| ATOM | 2676 | CA | VAL | A | 346 | 63.436 | 53.364 | 38.027 | 1.00 | 36.99 | | C |
| ANISOU | 2676 | CA | VAL | A | 346 | 5165 | 4532 | 4358 | −1013 | 1307 | −615 | C |
| ATOM | 2677 | CB | VAL | A | 346 | 64.426 | 53.540 | 39.218 | 1.00 | 39.70 | | C |
| ANISOU | 2677 | CB | VAL | A | 346 | 5599 | 4890 | 4595 | −1121 | 1245 | −743 | C |
| ATOM | 2678 | CG1 | VAL | A | 346 | 65.884 | 53.362 | 38.776 | 1.00 | 38.96 | | C |
| ANISOU | 2678 | CG1 | VAL | A | 346 | 5398 | 4923 | 4482 | −1210 | 1114 | −805 | C |
| ATOM | 2679 | CG2 | VAL | A | 346 | 64.250 | 54.903 | 39.876 | 1.00 | 39.36 | | C |
| ANISOU | 2679 | CG2 | VAL | A | 346 | 5715 | 4700 | 4539 | −1185 | 1373 | −809 | C |
| ATOM | 2680 | C | VAL | A | 346 | 63.552 | 52.002 | 37.323 | 1.00 | 41.75 | | C |
| ANISOU | 2680 | C | VAL | A | 346 | 5633 | 5263 | 4969 | −962 | 1192 | −553 | C |
| ATOM | 2681 | O | VAL | A | 346 | 62.806 | 51.070 | 37.650 | 1.00 | 42.47 | | O |
| ANISOU | 2681 | O | VAL | A | 346 | 5731 | 5381 | 5027 | −906 | 1186 | −515 | O |
| ATOM | 2682 | N | GLN | A | 347 | 64.414 | 51.924 | 36.304 | 1.00 | 36.88 | | N |
| ANISOU | 2682 | N | GLN | A | 347 | 4906 | 4714 | 4395 | −986 | 1119 | −542 | N |
| ATOM | 2683 | CA | GLN | A | 347 | 64.643 | 50.714 | 35.539 | 1.00 | 36.65 | | C |
| ANISOU | 2683 | CA | GLN | A | 347 | 4761 | 4796 | 4367 | −933 | 1011 | −489 | C |
| ATOM | 2684 | CB | GLN | A | 347 | 64.067 | 50.847 | 34.135 | 1.00 | 37.18 | | C |
| ANISOU | 2684 | CB | GLN | A | 347 | 4729 | 4860 | 4540 | −875 | 1058 | −395 | C |
| ATOM | 2685 | CG | GLN | A | 347 | 62.557 | 50.952 | 34.125 | 1.00 | 49.64 | | C |
| ANISOU | 2685 | CG | GLN | A | 347 | 6322 | 6386 | 6152 | −799 | 1168 | −332 | C |
| ATOM | 2686 | CD | GLN | A | 347 | 62.026 | 51.408 | 32.797 | 1.00 | 70.73 | | C |
| ANISOU | 2686 | CD | GLN | A | 347 | 8906 | 9059 | 8910 | −725 | 1224 | −254 | C |
| ATOM | 2687 | OE1 | GLN | A | 347 | 62.528 | 51.023 | 31.730 | 1.00 | 66.82 | | O |
| ANISOU | 2687 | OE1 | GLN | A | 347 | 8311 | 8629 | 8448 | −713 | 1155 | −217 | O |
| ATOM | 2688 | NE2 | GLN | A | 347 | 60.985 | 52.235 | 32.836 | 1.00 | 63.12 | | N |
| ANISOU | 2688 | NE2 | GLN | A | 347 | 7982 | 8030 | 7971 | −654 | 1351 | −227 | N |
| ATOM | 2689 | C | GLN | A | 347 | 66.135 | 50.448 | 35.515 | 1.00 | 44.10 | | C |
| ANISOU | 2689 | C | GLN | A | 347 | 5648 | 5854 | 5256 | −987 | 882 | −560 | C |
| ATOM | 2690 | O | GLN | A | 347 | 66.896 | 51.230 | 34.948 | 1.00 | 43.52 | | O |
| ANISOU | 2690 | O | GLN | A | 347 | 5517 | 5796 | 5222 | −1062 | 889 | −593 | O |
| ATOM | 2691 | N | ILE | A | 348 | 66.558 | 49.379 | 36.199 | 1.00 | 43.57 | | N |
| ANISOU | 2691 | N | ILE | A | 348 | 5606 | 5871 | 5078 | −947 | 772 | −590 | N |
| ATOM | 2692 | CA | ILE | A | 348 | 67.958 | 48.975 | 36.306 | 1.00 | 44.45 | | C |
| ANISOU | 2692 | CA | ILE | A | 348 | 5651 | 6135 | 5103 | −955 | 634 | −664 | C |
| ATOM | 2693 | CB | ILE | A | 348 | 68.357 | 48.710 | 37.780 | 1.00 | 47.29 | | C |
| ANISOU | 2693 | CB | ILE | A | 348 | 6117 | 6539 | 5314 | −947 | 570 | −754 | C |
| ATOM | 2694 | CG1 | ILE | A | 348 | 68.010 | 49.909 | 38.689 | 1.00 | 47.81 | | C |
| ANISOU | 2694 | CG1 | ILE | A | 348 | 6283 | 6496 | 5387 | −1052 | 675 | −822 | C |
| ATOM | 2695 | CD1 | ILE | A | 348 | 67.370 | 49.521 | 39.986 | 1.00 | 57.90 | | C |
| ANISOU | 2695 | CD1 | ILE | A | 348 | 7722 | 7710 | 6568 | −1006 | 697 | −833 | C |
| ATOM | 2696 | CG2 | ILE | A | 348 | 69.839 | 48.352 | 37.864 | 1.00 | 47.80 | | C |
| ANISOU | 2696 | CG2 | ILE | A | 348 | 6080 | 6810 | 5272 | −935 | 419 | −843 | C |
| ATOM | 2697 | C | ILE | A | 348 | 68.230 | 47.757 | 35.426 | 1.00 | 51.57 | | C |
| ANISOU | 2697 | C | ILE | A | 348 | 6474 | 7130 | 5991 | −851 | 538 | −598 | C |
| ATOM | 2698 | O | ILE | A | 348 | 67.651 | 46.693 | 35.651 | 1.00 | 52.30 | | O |
| ANISOU | 2698 | O | ILE | A | 348 | 6648 | 7192 | 6031 | −764 | 522 | −544 | O |
| ATOM | 2699 | N | ARG | A | 349 | 69.120 | 47.906 | 34.447 | 1.00 | 49.42 | | N |
| ANISOU | 2699 | N | ARG | A | 349 | 6060 | 6964 | 5756 | −867 | 487 | −607 | N |
| ATOM | 2700 | CA | ARG | A | 349 | 69.498 | 46.817 | 33.557 | 1.00 | 50.29 | | C |
| ANISOU | 2700 | CA | ARG | A | 349 | 6094 | 7169 | 5845 | −761 | 396 | −552 | C |
| ATOM | 2701 | CB | ARG | A | 349 | 68.454 | 46.613 | 32.431 | 1.00 | 49.73 | | C |
| ANISOU | 2701 | CB | ARG | A | 349 | 6005 | 7002 | 5888 | −732 | 469 | −438 | C |
| ATOM | 2702 | CG | ARG | A | 349 | 68.538 | 47.590 | 31.266 | 1.00 | 52.50 | | C |
| ANISOU | 2702 | CG | ARG | A | 349 | 6245 | 7342 | 6363 | −793 | 536 | −410 | C |
| ATOM | 2703 | CD | ARG | A | 349 | 67.432 | 47.311 | 30.274 | 1.00 | 56.56 | | C |
| ANISOU | 2703 | CD | ARG | A | 349 | 6742 | 7786 | 6961 | −737 | 593 | −304 | C |
| ATOM | 2704 | NE | ARG | A | 349 | 66.209 | 48.023 | 30.625 | 1.00 | 65.13 | | N |
| ANISOU | 2704 | NE | ARG | A | 349 | 7897 | 8747 | 8102 | −758 | 716 | −277 | N |
| ATOM | 2705 | CZ | ARG | A | 349 | 65.257 | 48.353 | 29.759 | 1.00 | 88.21 | | C |
| ANISOU | 2705 | CZ | ARG | A | 349 | 10783 | 11625 | 11108 | −721 | 794 | −203 | C |
| ATOM | 2706 | NH1 | ARG | A | 349 | 65.373 | 48.027 | 28.474 | 1.00 | 80.98 | | N |
| ANISOU | 2706 | NH1 | ARG | A | 349 | 9773 | 10764 | 10234 | −674 | 763 | −147 | N |
| ATOM | 2707 | NH2 | ARG | A | 349 | 64.179 | 49.012 | 30.169 | 1.00 | 74.54 | | N |
| ANISOU | 2707 | NH2 | ARG | A | 349 | 9106 | 9810 | 9407 | −714 | 903 | −188 | N |
| ATOM | 2708 | C | ARG | A | 349 | 70.910 | 47.024 | 33.011 | 1.00 | 57.39 | | C |
| ANISOU | 2708 | C | ARG | A | 349 | 6835 | 8251 | 6720 | −784 | 310 | −617 | C |
| ATOM | 2709 | O | ARG | A | 349 | 71.408 | 48.148 | 33.038 | 1.00 | 57.60 | | O |
| ANISOU | 2709 | O | ARG | A | 349 | 6799 | 8301 | 6785 | −920 | 354 | −689 | O |
| ATOM | 2710 | O | THR | A | 350 | 71.713 | 46.666 | 29.940 | 1.00 | 63.98 | | O |
| ANISOU | 2710 | O | THR | A | 350 | 7394 | 9227 | 7688 | −719 | 279 | −500 | O |
| ATOM | 2711 | N | THR | A | 350 | 71.547 | 45.947 | 32.512 | 1.00 | 55.45 | | N |
| ANISOU | 2711 | N | THR | A | 350 | 6533 | 8134 | 6402 | −656 | 200 | −596 | N |
| ATOM | 2712 | CA | THR | A | 350 | 72.888 | 46.005 | 31.927 | 1.00 | 55.87 | | C |
| ANISOU | 2712 | CA | THR | A | 350 | 6411 | 8397 | 6419 | −653 | 117 | −655 | C |
| ATOM | 2713 | C | THR | A | 350 | 72.816 | 46.397 | 30.438 | 1.00 | 63.66 | | C |
| ANISOU | 2713 | C | THR | A | 350 | 7289 | 9359 | 7538 | −697 | 179 | −587 | C |
| ATOM | 2714 | CB | THR | A | 350 | 73.634 | 44.694 | 32.162 | 1.00 | 57.38 | | C |
| ANISOU | 2714 | CB | THR | A | 350 | 6609 | 8747 | 6446 | −460 | −29 | −668 | C |
| ATOM | 2715 | OG1 | THR | A | 350 | 72.916 | 43.629 | 31.545 | 1.00 | 56.81 | | O |
| ANISOU | 2715 | OG1 | THR | A | 350 | 6638 | 8567 | 6382 | −335 | −27 | −555 | O |
| ATOM | 2716 | CG2 | THR | A | 350 | 73.849 | 44.401 | 33.627 | 1.00 | 54.25 | | C |
| ANISOU | 2716 | CG2 | THR | A | 350 | 6322 | 8394 | 5896 | −401 | −94 | −742 | C |
| ATOM | 2717 | N | ARG | A | 351A | 73.971 | 46.435 | 29.767 | 1.00 | 61.98 | | N |
| ANISOU | 2717 | N | ARG | A | 351A | 6907 | 9337 | 7305 | −703 | 125 | −630 | N |
| ATOM | 2718 | CA | ARG | A | 351A | 74.038 | 46.751 | 28.313 | 1.00 | 62.31 | | C |
| ANISOU | 2718 | CA | ARG | A | 351A | 6853 | 9368 | 7455 | −736 | 184 | −566 | C |
| ATOM | 2719 | CB | ARG | A | 351A | 75.495 | 46.904 | 27.869 | 1.00 | 61.80 | | C |
| ANISOU | 2719 | CB | ARG | A | 351A | 6589 | 9547 | 7345 | −776 | 133 | −646 | C |
| ATOM | 2720 | CG | ARG | A | 351A | 75.974 | 48.347 | 27.795 | 1.00 | 68.08 | | C |
| ANISOU | 2720 | CG | ARG | A | 351A | 7311 | 10350 | 8205 | −1014 | 241 | −725 | C |
| ATOM | 2721 | CD | ARG | A | 351A | 75.064 | 49.215 | 26.950 | 1.00 | 72.97 | | C |
| ANISOU | 2721 | CD | ARG | A | 351A | 8004 | 10751 | 8969 | −1100 | 395 | −632 | C |
| ATOM | 2722 | NE | ARG | A | 351A | 75.188 | 50.624 | 27.291 | 1.00 | 82.67 | | N |
| ANISOU | 2722 | NE | ARG | A | 351A | 9287 | 11891 | 10236 | −1321 | 517 | −709 | N |
| ATOM | 2723 | CZ | ARG | A | 351A | 74.491 | 51.597 | 26.720 | 1.00 | 96.43 | | C |
| ANISOU | 2723 | CZ | ARG | A | 351A | 11155 | 13406 | 12077 | −1398 | 671 | −646 | C |
| ATOM | 2724 | NH1 | ARG | A | 351A | 74.671 | 52.851 | 27.099 | 1.00 | 83.93 | | N |
| ANISOU | 2724 | NH1 | ARG | A | 351A | 9628 | 11693 | 10569 | −1267 | 712 | −507 | N |
| ATOM | 2725 | NH2 | ARG | A | 351A | 73.617 | 51.315 | 25.772 | 1.00 | 77.20 | | N |
| ANISOU | 2725 | NH2 | ARG | A | 351A | 8803 | 10876 | 9653 | −1596 | 787 | −726 | N |
| ATOM | 2726 | C | ARG | A | 351A | 73.348 | 45.642 | 27.507 | 1.00 | 67.08 | | C |
| ANISOU | 2726 | C | ARG | A | 351A | 7511 | 9903 | 8072 | −577 | 158 | −448 | C |
| ATOM | 2727 | O | ARG | A | 351A | 72.619 | 45.971 | 26.554 | 1.00 | 66.73 | | O |
| ANISOU | 2727 | O | ARG | A | 351A | 7469 | 9747 | 8137 | −598 | 239 | −364 | O |
| ATOM | 2728 | N | GLU | A | 352 | 73.574 | 44.381 | 27.891 | 1.00 | 63.54 | | N |
| ANISOU | 2728 | N | GLU | A | 352 | 7120 | 9527 | 7496 | −413 | 46 | −447 | N |
| ATOM | 2729 | CA | GLU | A | 352 | 72.998 | 43.193 | 27.190 | 1.00 | 63.31 | | C |
| ANISOU | 2729 | CA | GLU | A | 352 | 7180 | 9435 | 7439 | −266 | 12 | −356 | C |
| ATOM | 2730 | CB | GLU | A | 352 | 73.617 | 41.905 | 27.739 | 1.00 | 64.86 | | C |
| ANISOU | 2730 | CB | GLU | A | 352 | 7473 | 9719 | 7451 | −87 | −107 | −384 | C |
| ATOM | 2731 | CG | GLU | A | 352 | 74.174 | 40.994 | 26.661 | 1.00 | 81.29 | | C |
| ANISOU | 2731 | CG | GLU | A | 352 | 9706 | 11710 | 9470 | 60 | −134 | −302 | C |
| ATOM | 2732 | CD | GLU | A | 352 | 74.830 | 39.727 | 27.181 | 1.00 | 118.18 | | C |
| ANISOU | 2732 | CD | GLU | A | 352 | 14597 | 16171 | 14134 | 36 | −74 | −255 | C |
| ATOM | 2733 | OE1 | GLU | A | 352 | 76.029 | 39.528 | 26.903 | 1.00 | 116.58 | | O |
| ANISOU | 2733 | OE1 | GLU | A | 352 | 14446 | 15849 | 13999 | 6 | −18 | −185 | O |
| ATOM | 2734 | OE2 | GLU | A | 352 | 74.141 | 38.943 | 27.863 | 1.00 | 116.27 | | O |
| ANISOU | 2734 | OE2 | GLU | A | 352 | 14471 | 15898 | 13811 | 40 | −81 | −294 | O |
| ATOM | 2735 | C | GLU | A | 352 | 71.466 | 43.148 | 27.305 | 1.00 | 66.39 | | C |
| ANISOU | 2735 | C | GLU | A | 352 | 7699 | 9608 | 7920 | −318 | 108 | −280 | C |
| ATOM | 2736 | O | GLU | A | 352 | 70.810 | 42.907 | 26.273 | 1.00 | 66.14 | | O |
| ANISOU | 2736 | O | GLU | A | 352 | 7646 | 9513 | 7972 | −319 | 154 | −206 | O |
| ATOM | 2737 | N | MET | A | 353 | 70.924 | 43.370 | 28.508 | 1.00 | 62.04 | | N |
| ANISOU | 2737 | N | MET | A | 353 | 7265 | 8964 | 7344 | −363 | 141 | −306 | N |
| ATOM | 2738 | CA | MET | A | 353 | 69.474 | 43.417 | 28.740 | 1.00 | 61.14 | | C |
| ANISOU | 2738 | CA | MET | A | 353 | 7261 | 8677 | 7291 | −415 | 236 | −255 | C |
| ATOM | 2739 | CB | MET | A | 353 | 69.200 | 43.552 | 30.248 | 1.00 | 63.56 | | C |
| ANISOU | 2739 | CB | MET | A | 353 | 7686 | 8933 | 7532 | −450 | 252 | −311 | C |
| ATOM | 2740 | CG | MET | A | 353 | 69.886 | 42.521 | 31.116 | 1.00 | 66.88 | | C |
| ANISOU | 2740 | CG | MET | A | 353 | 8215 | 9417 | 7779 | −333 | 148 | −349 | C |
| ATOM | 2741 | SD | MET | A | 353 | 69.544 | 42.934 | 32.836 | 1.00 | 70.83 | | S |
| ANISOU | 2741 | SD | MET | A | 353 | 8861 | 9847 | 8204 | −380 | 182 | −409 | S |
| ATOM | 2742 | CE | MET | A | 353 | 70.830 | 41.993 | 33.682 | 1.00 | 67.38 | | C |
| ANISOU | 2742 | CE | MET | A | 353 | 8493 | 9568 | 7542 | −205 | 27 | −474 | C |
| ATOM | 2743 | C | MET | A | 353 | 68.780 | 44.571 | 27.988 | 1.00 | 62.22 | | C |
| ANISOU | 2743 | C | MET | A | 353 | 7317 | 8744 | 7580 | −508 | 346 | −215 | C |
| ATOM | 2744 | O | MET | A | 353 | 67.540 | 44.594 | 27.883 | 1.00 | 59.53 | | O |
| ANISOU | 2744 | O | MET | A | 353 | 7023 | 8302 | 7294 | −524 | 421 | −165 | O |
| ATOM | 2745 | N | HIS | A | 354 | 69.588 | 45.537 | 27.503 | 1.00 | 59.81 | | N |
| ANISOU | 2745 | N | HIS | A | 354 | 6896 | 8502 | 7328 | −565 | 362 | −243 | N |
| ATOM | 2746 | CA | HIS | A | 354 | 69.078 | 46.679 | 26.780 | 1.00 | 61.41 | | C |
| ANISOU | 2746 | CA | HIS | A | 354 | 7057 | 8627 | 7649 | −632 | 473 | −204 | C |
| ATOM | 2747 | CB | HIS | A | 354 | 69.929 | 47.929 | 26.999 | 1.00 | 62.50 | | C |
| ANISOU | 2747 | CB | HIS | A | 354 | 7154 | 8785 | 7808 | −754 | 521 | −276 | C |
| ATOM | 2748 | CG | HIS | A | 354 | 69.156 | 49.176 | 26.705 | 1.00 | 66.65 | | C |
| ANISOU | 2748 | CG | HIS | A | 354 | 7729 | 9168 | 8429 | −813 | 664 | −238 | C |
| ATOM | 2749 | ND1 | HIS | A | 354 | 69.684 | 50.186 | 25.927 | 1.00 | 68.91 | | N |
| ANISOU | 2749 | ND1 | HIS | A | 354 | 7980 | 9440 | 8764 | −892 | 740 | −241 | N |
| ATOM | 2750 | CE1 | HIS | A | 354 | 68.739 | 51.116 | 25.865 | 1.00 | 68.66 | | C |
| ANISOU | 2750 | CE1 | HIS | A | 354 | 8044 | 9252 | 8790 | −892 | 867 | −195 | C |
| ATOM | 2751 | NE2 | HIS | A | 354 | 67.635 | 50.745 | 26.524 | 1.00 | 68.84 | | N |
| ANISOU | 2751 | NE2 | HIS | A | 354 | 8129 | 9218 | 8811 | −816 | 871 | −166 | N |
| ATOM | 2752 | CD2 | HIS | A | 354 | 67.881 | 49.504 | 27.046 | 1.00 | 68.96 | | C |
| ANISOU | 2752 | CD2 | HIS | A | 354 | 8109 | 9331 | 8760 | −781 | 748 | −192 | C |
| ATOM | 2753 | C | HIS | A | 354 | 68.856 | 46.395 | 25.284 | 1.00 | 69.50 | | C |
| ANISOU | 2753 | C | HIS | A | 354 | 8009 | 9670 | 8729 | −568 | 476 | −121 | C |
| ATOM | 2754 | O | HIS | A | 354 | 69.816 | 46.174 | 24.524 | 1.00 | 70.84 | | O |
| ANISOU | 2754 | O | HIS | A | 354 | 8089 | 9946 | 8881 | −543 | 422 | −124 | O |
| ATOM | 2755 | O | ARG | A | 355 | 65.409 | 47.701 | 24.103 | 1.00 | 78.44 | | O |
| ANISOU | 2755 | O | ARG | A | 355 | 9171 | 10588 | 10045 | −490 | 723 | 54 | O |
| ATOM | 2756 | N | ARG | A | 355 | 67.569 | 46.380 | 24.872 | 1.00 | 65.64 | | N |
| ANISOU | 2756 | N | ARG | A | 355 | 7548 | 9098 | 8296 | −536 | 538 | −52 | N |
| ATOM | 2757 | CA | ARG | A | 355 | 67.129 | 46.122 | 23.491 | 1.00 | 65.14 | | C |
| ANISOU | 2757 | CA | ARG | A | 355 | 7422 | 9055 | 8274 | −469 | 541 | 23 | C |
| ATOM | 2758 | C | ARG | A | 355 | 65.933 | 47.061 | 23.155 | 1.00 | 73.07 | | C |
| ANISOU | 2758 | C | ARG | A | 355 | 8428 | 9982 | 9355 | −456 | 654 | 76 | C |
| ATOM | 2759 | CB | ARG | A | 355 | 66.780 | 44.617 | 23.278 | 1.00 | 63.03 | | C |
| ANISOU | 2759 | CB | ARG | A | 355 | 7181 | 8823 | 7945 | −406 | 459 | 42 | C |
| ATOM | 2760 | CG | ARG | A | 355 | 67.970 | 43.640 | 23.332 | 1.00 | 68.86 | | C |
| ANISOU | 2760 | CG | ARG | A | 355 | 7928 | 9645 | 8590 | −355 | 348 | 10 | C |
| ATOM | 2761 | CD | ARG | A | 355 | 67.542 | 42.176 | 23.243 | 1.00 | 87.77 | | C |
| ANISOU | 2761 | CD | ARG | A | 355 | 10417 | 12026 | 10906 | −297 | 292 | 27 | C |
| ATOM | 2762 | NE | ARG | A | 355 | 68.482 | 41.371 | 22.448 | 1.00 | 108.80 | | N |
| ANISOU | 2762 | NE | ARG | A | 355 | 13064 | 14773 | 13504 | −202 | 205 | 35 | N |
| ATOM | 2763 | CZ | ARG | A | 355 | 68.144 | 40.508 | 21.483 | 1.00 | 120.60 | | C |
| ANISOU | 2763 | CZ | ARG | A | 355 | 14583 | 16264 | 14974 | −150 | 179 | 74 | C |
| ATOM | 2764 | NH1 | ARG | A | 355 | 69.083 | 39.841 | 20.821 | 1.00 | 101.54 | | N |
| ANISOU | 2764 | NH1 | ARG | A | 355 | 12164 | 13925 | 12491 | −47 | 105 | 77 | N |
| ATOM | 2765 | NH2 | ARG | A | 355 | 66.865 | 40.311 | 21.172 | 1.00 | 103.56 | | N |
| ANISOU | 2765 | NH2 | ARG | A | 355 | 12450 | 14046 | 12852 | −202 | 229 | 101 | N |
| ATOM | 2766 | OXT | ARG | A | 355 | 65.546 | 47.179 | 21.962 | 1.00 | 73.83 | | O |
| ANISOU | 2766 | OXT | ARG | A | 355 | 8467 | 10099 | 9487 | −395 | 673 | 137 | O |
| TER | 2767 | | ARG | A | 355 | | | | | | | |
| HETATM | 2768 | MN | MN | B | 1 | 67.628 | 48.309 | 2.819 | 1.00 | 74.59 | | MN |
| HETATM | 2769 | O3D | G4P | C | 1 | 64.559 | 45.249 | 0.538 | 0.40 | 180.06 | | O |
| HETATM | 2770 | PD | G4P | C | 1 | 64.264 | 46.123 | −0.658 | 0.40 | 180.39 | | P |
| HETATM | 2771 | O1D | G4P | C | 1 | 65.399 | 46.208 | −1.649 | 0.40 | 180.10 | | O |
| HETATM | 2772 | O2D | G4P | C | 1 | 62.906 | 45.904 | −1.278 | 0.40 | 180.06 | | O |
| HETATM | 2773 | O3C | G4P | C | 1 | 64.178 | 47.609 | −0.045 | 0.40 | 181.87 | | O |
| HETATM | 2774 | PC | G4P | C | 1 | 63.205 | 48.715 | −0.691 | 0.40 | 183.70 | | P |
| HETATM | 2775 | O1C | G4P | C | 1 | 62.945 | 48.344 | −2.130 | 0.40 | 183.65 | | O |
| HETATM | 2776 | O2C | G4P | C | 1 | 62.048 | 48.944 | 0.252 | 0.40 | 183.48 | | O |
| HETATM | 2777 | O3′ | G4P | C | 1 | 64.138 | 50.023 | −0.679 | 1.00 | 185.78 | | O |
| HETATM | 2778 | C3′ | G4P | C | 1 | 63.591 | 51.339 | −0.660 | 1.00 | 186.99 | | C |
| HETATM | 2779 | C4′ | G4P | C | 1 | 63.321 | 51.791 | −2.083 | 1.00 | 190.08 | | C |
| HETATM | 2780 | C5′ | G4P | C | 1 | 61.843 | 52.015 | −2.332 | 1.00 | 197.09 | | C |
| HETATM | 2781 | O5′ | G4P | C | 1 | 61.530 | 51.439 | −3.592 | 1.00 | 203.76 | | O |
| HETATM | 2782 | PA | G4P | C | 1 | 59.999 | 51.332 | −4.056 | 1.00 | 209.12 | | P |
| HETATM | 2783 | O1A | G4P | C | 1 | 59.966 | 50.940 | −5.509 | 1.00 | 210.34 | | O |
| HETATM | 2784 | O2A | G4P | C | 1 | 59.244 | 52.547 | −3.583 | 1.00 | 209.72 | | O |
| HETATM | 2785 | O3A | G4P | C | 1 | 59.544 | 50.054 | −3.206 | 1.00 | 209.50 | | O |
| HETATM | 2786 | PB | G4P | C | 1 | 59.447 | 48.649 | −3.962 | 1.00 | 209.20 | | P |
| HETATM | 2787 | O3B | G4P | C | 1 | 58.249 | 48.815 | −4.862 | 1.00 | 208.49 | | O |
| HETATM | 2788 | O2B | G4P | C | 1 | 59.266 | 47.647 | −2.850 | 1.00 | 209.44 | | O |
| HETATM | 2789 | O1B | G4P | C | 1 | 60.766 | 48.557 | −4.687 | 1.00 | 209.08 | | O |
| HETATM | 2790 | C2′ | G4P | C | 1 | 64.584 | 52.346 | −0.109 | 1.00 | 184.21 | | C |
| HETATM | 2791 | O2′ | G4P | C | 1 | 65.710 | 51.747 | 0.541 | 1.00 | 183.77 | | O |
| HETATM | 2792 | C1′ | G4P | C | 1 | 65.069 | 53.109 | −1.321 | 1.00 | 181.56 | | C |
| HETATM | 2793 | O4′ | G4P | C | 1 | 64.020 | 53.019 | −2.273 | 1.00 | 185.56 | | O |
| HETATM | 2794 | N9 | G4P | C | 1 | 65.270 | 54.501 | −0.904 | 1.00 | 175.49 | | N |
| HETATM | 2795 | C4 | G4P | C | 1 | 64.597 | 55.093 | 0.068 | 1.00 | 172.47 | | C |
| HETATM | 2796 | C5 | G4P | C | 1 | 65.130 | 56.447 | 0.173 | 1.00 | 171.61 | | C |
| HETATM | 2797 | N7 | G4P | C | 1 | 66.112 | 56.538 | −0.738 | 1.00 | 172.57 | | N |
| HETATM | 2798 | C8 | G4P | C | 1 | 66.188 | 55.350 | −1.382 | 1.00 | 173.60 | | C |
| HETATM | 2799 | N3 | G4P | C | 1 | 63.588 | 54.729 | 0.878 | 1.00 | 170.68 | | N |
| HETATM | 2800 | C2 | G4P | C | 1 | 63.083 | 55.594 | 1.785 | 1.00 | 169.20 | | C |
| HETATM | 2801 | N2 | G4P | C | 1 | 62.073 | 55.167 | 2.572 | 1.00 | 168.87 | | N |
| HETATM | 2802 | N1 | G4P | C | 1 | 63.535 | 56.845 | 1.935 | 1.00 | 168.96 | | N |
| HETATM | 2803 | C6 | G4P | C | 1 | 64.525 | 57.328 | 1.187 | 1.00 | 169.79 | | C |
| HETATM | 2804 | O6 | G4P | C | 1 | 64.932 | 58.494 | 1.337 | 1.00 | 169.27 | | O |
| HETATM | 2805 | N | ARG | D | 1 | 65.353 | 55.400 | −5.166 | 1.00 | 140.03 | | N |
| HETATM | 2806 | CA | ARG | D | 1 | 66.433 | 55.455 | −6.188 | 1.00 | 142.92 | | C |
| HETATM | 2807 | C | ARG | D | 1 | 66.321 | 56.768 | −6.972 | 1.00 | 151.16 | | C |
| HETATM | 2808 | O | ARG | D | 1 | 65.222 | 57.102 | −7.415 | 1.00 | 150.87 | | O |
| HETATM | 2809 | CB | ARG | D | 1 | 66.344 | 54.246 | −7.124 | 1.00 | 143.06 | | C |
| HETATM | 2810 | CG | ARG | D | 1 | 66.145 | 52.918 | −6.407 | 1.00 | 151.43 | | C |
| HETATM | 2811 | CD | ARG | D | 1 | 66.291 | 51.731 | −7.340 | 1.00 | 158.24 | | C |
| HETATM | 2812 | NE | ARG | D | 1 | 66.532 | 50.488 | −6.621 | 1.00 | 164.17 | | N |
| HETATM | 2813 | CZ | ARG | D | 1 | 65.803 | 50.057 | −5.597 | 1.00 | 176.06 | | C |
| HETATM | 2814 | NH1 | ARG | D | 1 | 64.778 | 50.772 | −5.168 | 1.00 | 162.79 | | N |
| HETATM | 2815 | NH2 | ARG | D | 1 | 66.102 | 48.914 | −5.008 | 1.00 | 160.80 | | N |
| HETATM | 2816 | OXT | ARG | D | 1 | 67.355 | 57.433 | −7.123 | 1.00 | 168.58 | | O |
| HETATM | 2818 | NA | NA | E | 1 | 83.212 | 44.223 | −5.080 | 1.00 | 37.91 | | NA1+ |
| HETATM | 2819 | O | HOH | W | 1 | 80.797 | 47.711 | 26.220 | 1.00 | 38.71 | | O |
| HETATM | 2820 | O | HOH | W | 2 | 65.346 | 59.709 | 17.664 | 1.00 | 44.69 | | O |
| HETATM | 2821 | O | HOH | W | 3 | 56.724 | 50.775 | 23.198 | 1.00 | 31.32 | | O |
| HETATM | 2822 | O | HOH | W | 4 | 79.017 | 49.744 | −8.217 | 1.00 | 39.44 | | O |
| HETATM | 2823 | O | HOH | W | 5 | 85.819 | 53.221 | −9.039 | 1.00 | 46.34 | | O |
| HETATM | 2824 | O | HOH | W | 6 | 65.823 | 36.020 | 28.531 | 1.00 | 45.26 | | O |
| HETATM | 2825 | O | HOH | W | 7 | 64.898 | 39.395 | 24.076 | 1.00 | 40.60 | | O |
| HETATM | 2826 | O | HOH | W | 8 | 62.535 | 36.469 | 22.495 | 1.00 | 57.59 | | O |
| HETATM | 2827 | O | HOH | W | 9 | 55.824 | 39.730 | 26.426 | 1.00 | 36.57 | | O |
| HETATM | 2828 | O | HOH | W | 10 | 69.736 | 57.927 | 3.585 | 1.00 | 43.95 | | O |
| HETATM | 2829 | O | HOH | W | 11 | 53.796 | 37.655 | 16.334 | 1.00 | 19.95 | | O |
| HETATM | 2830 | O | HOH | W | 12 | 74.026 | 43.580 | −11.117 | 1.00 | 41.86 | | O |
| HETATM | 2831 | O | HOH | W | 13 | 54.153 | 64.083 | 9.753 | 1.00 | 32.74 | | O |
| HETATM | 2832 | O | HOH | W | 14 | 52.710 | 65.976 | 12.005 | 1.00 | 30.98 | | O |
| HETATM | 2833 | O | HOH | W | 15 | 73.133 | 47.785 | 24.130 | 1.00 | 44.63 | | O |
| HETATM | 2834 | O | HOH | W | 16 | 66.361 | 37.167 | 24.749 | 1.00 | 81.18 | | O |
| HETATM | 2835 | O | HOH | W | 17 | 65.223 | 41.792 | 25.702 | 1.00 | 29.94 | | O |
| HETATM | 2836 | O | HOH | W | 18 | 87.281 | 54.930 | 3.892 | 1.00 | 60.97 | | O |
| HETATM | 2837 | O | HOH | W | 19 | 90.371 | 45.316 | −1.396 | 1.00 | 30.56 | | O |
| HETATM | 2838 | O | HOH | W | 20 | 89.325 | 47.159 | 1.194 | 1.00 | 17.67 | | O |
| HETATM | 2839 | O | HOH | W | 21 | 70.407 | 54.340 | −8.198 | 1.00 | 45.61 | | O |
| HETATM | 2840 | O | HOH | W | 22 | 78.200 | 57.068 | −4.443 | 1.00 | 36.27 | | O |
| HETATM | 2841 | O | HOH | W | 23 | 40.555 | 41.081 | 12.565 | 1.00 | 56.39 | | O |
| HETATM | 2842 | O | HOH | W | 24 | 63.015 | 63.123 | 11.365 | 1.00 | 39.23 | | O |
| HETATM | 2843 | O | HOH | W | 25 | 63.487 | 59.332 | 15.472 | 1.00 | 56.07 | | O |
| HETATM | 2844 | O | HOH | W | 26 | 65.989 | 64.553 | 12.586 | 1.00 | 57.29 | | O |
| HETATM | 2845 | O | HOH | W | 27 | 83.396 | 53.807 | 35.437 | 1.00 | 52.30 | | O |
| HETATM | 2846 | O | HOH | W | 28 | 84.290 | 47.627 | 25.812 | 1.00 | 34.57 | | O |
| HETATM | 2847 | O | HOH | W | 29 | 81.797 | 44.598 | 25.409 | 1.00 | 38.33 | | O |
| HETATM | 2848 | O | HOH | W | 30 | 82.600 | 39.029 | 40.298 | 1.00 | 44.48 | | O |
| HETATM | 2849 | O | HOH | W | 31 | 57.879 | 49.664 | −8.016 | 1.00 | 59.08 | | O |
| HETATM | 2850 | O | HOH | W | 32 | 69.815 | 50.795 | −8.510 | 1.00 | 52.71 | | O |
| HETATM | 2851 | O | HOH | W | 33 | 74.938 | 27.999 | 6.002 | 1.00 | 47.91 | | O |
| HETATM | 2852 | O | HOH | W | 34 | 72.077 | 44.158 | 23.650 | 1.00 | 32.69 | | O |
| HETATM | 2853 | O | HOH | W | 35 | 67.034 | 54.594 | 27.568 | 1.00 | 42.25 | | O |
| HETATM | 2854 | O | HOH | W | 36 | 54.612 | 58.180 | 31.155 | 1.00 | 76.86 | | O |
| HETATM | 2855 | O | HOH | W | 37 | 50.614 | 60.442 | 38.118 | 1.00 | 53.89 | | O |
| HETATM | 2856 | O | HOH | W | 38 | 43.135 | 51.259 | 4.478 | 1.00 | 53.83 | | O |
| HETATM | 2857 | O | HOH | W | 39 | 43.367 | 53.189 | −2.288 | 1.00 | 41.07 | | O |
| HETATM | 2858 | O | HOH | W | 40 | 46.931 | 47.528 | −2.561 | 1.00 | 47.69 | | O |
| HETATM | 2859 | O | HOH | W | 41 | 61.768 | 48.313 | 4.712 | 1.00 | 44.43 | | O |
| HETATM | 2860 | O | HOH | W | 42 | 58.814 | 44.875 | 3.617 | 1.00 | 56.52 | | O |
| HETATM | 2861 | O | HOH | W | 43 | 41.651 | 37.498 | 4.845 | 1.00 | 45.41 | | O |
| HETATM | 2862 | O | HOH | W | 44 | 64.902 | 64.046 | 45.347 | 1.00 | 34.46 | | O |
| HETATM | 2863 | O | HOH | W | 45 | 56.219 | 37.742 | 23.111 | 1.00 | 34.71 | | O |
| HETATM | 2864 | O | HOH | W | 46 | 70.713 | 43.103 | −1.765 | 1.00 | 77.08 | | O |
| HETATM | 2865 | O | HOH | W | 47 | 82.545 | 50.563 | −7.699 | 1.00 | 36.84 | | O |
| HETATM | 2866 | O | HOH | W | 48 | 82.249 | 43.205 | −7.318 | 1.00 | 46.35 | | O |
| HETATM | 2867 | O | HOH | W | 49 | 70.951 | 42.871 | 21.519 | 1.00 | 40.79 | | O |
| HETATM | 2868 | O | HOH | W | 50 | 85.459 | 51.910 | 36.873 | 1.00 | 45.79 | | O |
| HETATM | 2869 | O | HOH | W | 51 | 42.687 | 56.204 | 32.181 | 1.00 | 65.43 | | O |
| HETATM | 2870 | O | HOH | W | 52 | 44.158 | 53.464 | 33.701 | 1.00 | 70.53 | | O |
| HETATM | 2871 | O | HOH | W | 53 | 43.416 | 50.906 | 16.138 | 1.00 | 54.06 | | O |
| HETATM | 2872 | O | HOH | W | 54 | 60.149 | 61.053 | −4.657 | 1.00 | 51.05 | | O |
| HETATM | 2873 | O | HOH | W | 55 | 63.767 | 45.928 | 26.035 | 1.00 | 32.24 | | O |
| HETATM | 2874 | O | HOH | W | 56 | 60.168 | 44.883 | 25.300 | 1.00 | 41.92 | | O |
| HETATM | 2875 | O | HOH | W | 57 | 54.680 | 56.278 | 28.016 | 1.00 | 65.79 | | O |
| HETATM | 2876 | O | HOH | W | 58 | 69.383 | 39.063 | 26.972 | 1.00 | 70.37 | | O |
| HETATM | 2877 | O | HOH | W | 59 | 62.441 | 54.620 | −4.809 | 1.00 | 74.63 | | O |
| HETATM | 2878 | O | HOH | W | 60 | 58.747 | 39.643 | 39.034 | 1.00 | 38.58 | | O |
| HETATM | 2879 | O | HOH | W | 61 | 52.661 | 31.878 | 7.452 | 1.00 | 35.22 | | O |
| HETATM | 2880 | O | HOH | W | 62 | 48.363 | 37.890 | 0.370 | 1.00 | 47.86 | | O |
| HETATM | 2881 | O | HOH | W | 63 | 42.822 | 37.898 | 1.408 | 1.00 | 32.26 | | O |
| HETATM | 2882 | O | HOH | W | 64 | 79.418 | 32.427 | −8.205 | 1.00 | 33.43 | | O |
| HETATM | 2883 | O | HOH | W | 65 | 56.555 | 47.643 | 2.093 | 1.00 | 44.49 | | O |
| HETATM | 2884 | O | HOH | W | 66 | 67.858 | 48.232 | −7.973 | 1.00 | 82.74 | | O |
| HETATM | 2885 | O | HOH | W | 67 | 70.243 | 51.886 | −5.798 | 1.00 | 31.38 | | O |
| HETATM | 2886 | O | HOH | W | 68 | 72.367 | 51.174 | −9.749 | 1.00 | 45.94 | | O |
| HETATM | 2887 | O | HOH | W | 69 | 63.021 | 48.638 | 26.516 | 1.00 | 35.36 | | O |
| HETATM | 2888 | O | HOH | W | 70 | 57.221 | 48.084 | −10.754 | 1.00 | 63.68 | | O |
| HETATM | 2889 | O | HOH | W | 71 | 60.860 | 46.805 | 1.706 | 1.00 | 57.50 | | O |
| HETATM | 2890 | O | HOH | W | 72 | 85.606 | 40.687 | 41.737 | 1.00 | 43.49 | | O |
| HETATM | 2891 | O | HOH | W | 73 | 78.070 | 56.119 | 47.688 | 1.00 | 39.30 | | O |
| HETATM | 2892 | O | HOH | W | 74 | 41.165 | 51.871 | 33.820 | 1.00 | 66.75 | | O |
| HETATM | 2893 | O | HOH | W | 75 | 71.681 | 37.108 | −5.458 | 1.00 | 39.83 | | O |
| HETATM | 2894 | O | HOH | W | 76 | 69.609 | 35.852 | −3.206 | 1.00 | 51.70 | | O |
| HETATM | 2895 | O | HOH | W | 77 | 79.535 | 46.334 | −7.773 | 1.00 | 35.69 | | O |
| HETATM | 2896 | O | HOH | W | 78 | 58.820 | 48.645 | 0.342 | 1.00 | 60.73 | | O |
| HETATM | 2897 | O | HOH | W | 79 | 60.364 | 50.696 | 1.664 | 1.00 | 54.93 | | O |
| HETATM | 2898 | O | HOH | W | 80 | 67.556 | 44.548 | 1.184 | 1.00 | 50.82 | | O |
| HETATM | 2899 | O | HOH | W | 81 | 62.393 | 50.423 | −7.561 | 1.00 | 75.31 | | O |
| HETATM | 2900 | O | HOH | W | 82 | 61.866 | 53.511 | −7.581 | 1.00 | 57.63 | | O |
| HETATM | 2901 | O | HOH | W | 83 | 81.109 | 35.359 | −6.936 | 1.00 | 32.29 | | O |
| HETATM | 2902 | O | HOH | W | 84 | 87.952 | 42.025 | 39.183 | 1.00 | 67.84 | | O |
| HETATM | 2903 | O | HOH | W | 85 | 75.446 | 59.241 | 31.594 | 1.00 | 67.88 | | O |
| HETATM | 2904 | O | HOH | W | 86 | 78.845 | 58.338 | 32.738 | 1.00 | 45.56 | | O |
| HETATM | 2905 | O | HOH | W | 87 | 70.055 | 47.379 | −9.368 | 1.00 | 49.36 | | O |
| HETATM | 2906 | O | HOH | W | 88 | 67.502 | 44.775 | −2.287 | 1.00 | 106.44 | | O |
| HETATM | 2907 | O | HOH | W | 89 | 66.726 | 65.750 | −3.806 | 1.00 | 59.49 | | O |
| HETATM | 2908 | O | HOH | W | 90 | 55.750 | 53.812 | 21.562 | 1.00 | 36.15 | | O |
| HETATM | 2909 | O | HOH | W | 91 | 66.759 | 51.836 | 18.235 | 1.00 | 42.47 | | O |
| HETATM | 2910 | O | HOH | W | 92 | 69.979 | 52.217 | 17.965 | 1.00 | 51.42 | | O |
| HETATM | 2911 | O | HOH | W | 93 | 68.018 | 50.466 | 30.213 | 1.00 | 131.92 | | O |
| HETATM | 2913 | O | HOH | W | 94 | 68.187 | 58.996 | 2.286 | 1.00 | 30.00 | | O |
| HETATM | 2914 | O | HOH | W | 95 | 62.548 | 46.749 | −5.662 | 1.00 | 30.00 | | O |
| HETATM | 2915 | O | HOH | W | 96 | 65.130 | 46.839 | −4.509 | 1.00 | 30.00 | | O |
| END | | | | | | | | | | | | |

TABLE 4
Rel allosteric site crystal structure atomic coordinates:

| | | | | | | | | | | | | |
|---|---|---|---|---|---|---|---|---|---|---|---|---|
| ATOM | 1 | N | ASP | A | 16 | 10.554 | 7.836 | −15.584 | 1.00 | 10.00 | A | N |
| ATOM | 3 | CA | ASP | A | 16 | 11.633 | 7.950 | −14.615 | 1.00 | 10.00 | A | C |
| ATOM | 4 | CB | ASP | A | 16 | 12.950 | 8.363 | −15.293 | 1.00 | 10.00 | A | C |
| ATOM | 5 | CG | ASP | A | 16 | 12.877 | 9.685 | −16.038 | 1.00 | 10.00 | A | C |
| ATOM | 6 | OD1 | ASP | A | 16 | 12.810 | 10.741 | −15.382 | 1.00 | 10.00 | A | O |
| ATOM | 7 | OD2 | ASP | A | 16 | 12.908 | 9.670 | −17.290 | 1.00 | 10.00 | A | O |
| ATOM | 8 | C | ASP | A | 16 | 11.253 | 8.931 | −13.509 | 1.00 | 10.00 | A | C |
| ATOM | 9 | O | ASP | A | 16 | 10.442 | 9.833 | −13.730 | 1.00 | 10.00 | A | O |
| ATOM | 10 | N | PRO | A | 17 | 11.814 | 8.752 | −12.300 | 1.00 | 10.00 | A | N |
| ATOM | 11 | CA | PRO | A | 17 | 11.534 | 9.619 | −11.151 | 1.00 | 10.00 | A | C |
| ATOM | 12 | CB | PRO | A | 17 | 12.434 | 9.067 | −10.038 | 1.00 | 10.00 | A | C |
| ATOM | 13 | CG | PRO | A | 17 | 13.443 | 8.222 | −10.735 | 1.00 | 10.00 | A | C |
| ATOM | 14 | CD | PRO | A | 17 | 12.755 | 7.678 | −11.949 | 1.00 | 10.00 | A | C |
| ATOM | 15 | C | PRO | A | 17 | 11.862 | 11.086 | −11.425 | 1.00 | 10.00 | A | C |
| ATOM | 16 | O | PRO | A | 17 | 11.203 | 11.976 | −10.890 | 1.00 | 10.00 | A | O |
| ATOM | 17 | N | GLU | A | 18 | 12.855 | 11.328 | −12.279 | 1.00 | 10.00 | A | N |
| ATOM | 19 | CA | GLU | A | 18 | 13.264 | 12.691 | −12.621 | 1.00 | 10.00 | A | C |
| ATOM | 20 | CB | GLU | A | 18 | 14.481 | 12.676 | −13.547 | 1.00 | 10.00 | A | C |
| ATOM | 21 | CG | GLU | A | 18 | 15.745 | 13.248 | −12.928 | 1.00 | 10.00 | A | C |
| ATOM | 22 | CD | GLU | A | 18 | 16.509 | 14.135 | −13.890 | 1.00 | 10.00 | A | C |
| ATOM | 23 | OE1 | GLU | A | 18 | 16.318 | 15.369 | −13.849 | 1.00 | 10.00 | A | O |
| ATOM | 24 | OE2 | GLU | A | 18 | 17.293 | 13.608 | −14.706 | 1.00 | 10.00 | A | O |
| ATOM | 25 | C | GLU | A | 18 | 12.122 | 13.463 | −13.277 | 1.00 | 10.00 | A | C |
| ATOM | 26 | O | GLU | A | 18 | 12.023 | 14.682 | −13.136 | 1.00 | 10.00 | A | O |
| ATOM | 27 | N | LYS | A | 19 | 11.278 | 12.753 | −14.010 | 1.00 | 10.00 | A | N |
| ATOM | 29 | CA | LYS | A | 19 | 10.137 | 13.365 | −14.674 | 1.00 | 10.00 | A | C |
| ATOM | 30 | CB | LYS | A | 19 | 9.979 | 12.808 | −16.089 | 1.00 | 10.00 | A | C |
| ATOM | 31 | CG | LYS | A | 19 | 11.144 | 13.114 | −17.017 | 1.00 | 10.00 | A | C |
| ATOM | 32 | CD | LYS | A | 19 | 11.252 | 14.602 | −17.305 | 1.00 | 10.00 | A | C |
| ATOM | 33 | CE | LYS | A | 19 | 12.565 | 14.941 | −17.989 | 1.00 | 10.00 | A | C |
| ATOM | 34 | NZ | LYS | A | 19 | 12.773 | 14.144 | −19.226 | 1.00 | 10.00 | A | N |
| ATOM | 38 | C | LYS | A | 19 | 8.856 | 13.140 | −13.872 | 1.00 | 10.00 | A | C |
| ATOM | 39 | O | LYS | A | 19 | 7.840 | 13.793 | −14.109 | 1.00 | 10.00 | A | O |
| ATOM | 40 | N | TRP | A | 20 | 8.927 | 12.225 | −12.912 | 1.00 | 10.00 | A | N |
| ATOM | 42 | CA | TRP | A | 20 | 7.789 | 11.896 | −12.073 | 1.00 | 10.00 | A | C |
| ATOM | 43 | CB | TRP | A | 20 | 7.970 | 10.504 | −11.462 | 1.00 | 10.00 | A | C |
| ATOM | 44 | CG | TRP | A | 20 | 6.786 | 9.602 | −11.650 | 1.00 | 10.00 | A | C |
| ATOM | 45 | CD1 | TRP | A | 20 | 6.720 | 8.492 | −12.445 | 1.00 | 10.00 | A | C |
| ATOM | 46 | CD2 | TRP | A | 20 | 5.495 | 9.729 | −11.034 | 1.00 | 10.00 | A | C |
| ATOM | 47 | NE1 | TRP | A | 20 | 5.471 | 7.920 | −12.358 | 1.00 | 10.00 | A | N |
| ATOM | 49 | CE2 | TRP | A | 20 | 4.702 | 8.663 | −11.501 | 1.00 | 10.00 | A | C |
| ATOM | 50 | CE3 | TRP | A | 20 | 4.932 | 10.640 | −10.133 | 1.00 | 10.00 | A | C |
| ATOM | 51 | CZ2 | TRP | A | 20 | 3.381 | 8.484 | −11.099 | 1.00 | 10.00 | A | C |
| ATOM | 52 | CZ3 | TRP | A | 20 | 3.621 | 10.457 | −9.737 | 1.00 | 10.00 | A | C |
| ATOM | 53 | CH2 | TRP | A | 20 | 2.861 | 9.390 | −10.219 | 1.00 | 10.00 | A | C |
| ATOM | 54 | C | TRP | A | 20 | 7.602 | 12.923 | −10.966 | 1.00 | 10.00 | A | C |
| ATOM | 55 | O | TRP | A | 20 | 6.540 | 13.539 | −10.855 | 1.00 | 10.00 | A | O |
| ATOM | 56 | N | ILE | A | 21 | 8.639 | 13.123 | −10.161 | 1.00 | 10.00 | A | N |
| ATOM | 58 | CA | ILE | A | 21 | 8.568 | 14.062 | −9.046 | 1.00 | 10.00 | A | C |
| ATOM | 59 | CB | ILE | A | 21 | 9.690 | 13.830 | −8.009 | 1.00 | 10.00 | A | C |
| ATOM | 60 | CG1 | ILE | A | 21 | 9.377 | 14.555 | −6.695 | 1.00 | 10.00 | A | C |
| ATOM | 61 | CG2 | ILE | A | 21 | 11.043 | 14.269 | −8.549 | 1.00 | 10.00 | A | C |
| ATOM | 62 | CD1 | ILE | A | 21 | 8.169 | 14.014 | −5.962 | 1.00 | 10.00 | A | C |
| ATOM | 63 | C | ILE | A | 21 | 8.572 | 15.510 | −9.528 | 1.00 | 10.00 | A | C |
| ATOM | 64 | O | ILE | A | 21 | 8.108 | 16.403 | −8.829 | 1.00 | 10.00 | A | O |
| ATOM | 65 | N | ALA | A | 22 | 9.076 | 15.731 | −10.736 | 1.00 | 10.00 | A | N |
| ATOM | 67 | CA | ALA | A | 22 | 9.131 | 17.071 | −11.315 | 1.00 | 10.00 | A | C |
| ATOM | 68 | CB | ALA | A | 22 | 9.822 | 17.034 | −12.669 | 1.00 | 10.00 | A | C |
| ATOM | 69 | C | ALA | A | 22 | 7.736 | 17.674 | −11.453 | 1.00 | 10.00 | A | C |
| ATOM | 70 | O | ALA | A | 22 | 7.570 | 18.893 | −11.425 | 1.00 | 10.00 | A | O |
| ATOM | 71 | N | SER | A | 23 | 6.739 | 16.810 | −11.593 | 1.00 | 10.00 | A | N |
| ATOM | 73 | CA | SER | A | 23 | 5.361 | 17.241 | −11.742 | 1.00 | 10.00 | A | C |
| ATOM | 74 | CB | SER | A | 23 | 4.600 | 16.206 | −12.575 | 1.00 | 10.00 | A | C |
| ATOM | 75 | OG | SER | A | 23 | 5.436 | 15.655 | −13.589 | 1.00 | 10.00 | A | O |
| ATOM | 77 | C | SER | A | 23 | 4.675 | 17.426 | −10.384 | 1.00 | 10.00 | A | C |
| ATOM | 78 | O | SER | A | 23 | 3.526 | 17.870 | −10.312 | 1.00 | 10.00 | A | O |
| ATOM | 79 | N | LEU | A | 24 | 5.376 | 17.090 | −9.305 | 1.00 | 10.00 | A | N |
| ATOM | 81 | CA | LEU | A | 24 | 4.811 | 17.215 | −7.966 | 1.00 | 10.00 | A | C |
| ATOM | 82 | CB | LEU | A | 24 | 4.616 | 15.834 | −7.327 | 1.00 | 10.00 | A | C |
| ATOM | 83 | CG | LEU | A | 24 | 3.323 | 15.080 | −7.669 | 1.00 | 10.00 | A | C |
| ATOM | 84 | CD1 | LEU | A | 24 | 2.108 | 15.993 | −7.579 | 1.00 | 10.00 | A | C |
| ATOM | 85 | CD2 | LEU | A | 24 | 3.408 | 14.409 | −9.034 | 1.00 | 10.00 | A | C |
| ATOM | 86 | C | LEU | A | 24 | 5.677 | 18.087 | −7.054 | 1.00 | 10.00 | A | C |
| ATOM | 87 | O | LEU | A | 24 | 5.245 | 18.467 | −5.963 | 1.00 | 10.00 | A | O |
| ATOM | 88 | N | GLY | A | 25 | 6.892 | 18.393 | −7.496 | 1.00 | 10.00 | A | N |
| ATOM | 90 | CA | GLY | A | 25 | 7.792 | 19.202 | −6.701 | 1.00 | 10.00 | A | C |
| ATOM | 91 | C | GLY | A | 25 | 8.254 | 20.445 | −7.431 | 1.00 | 10.00 | A | C |
| ATOM | 92 | O | GLY | A | 25 | 8.153 | 20.533 | −8.655 | 1.00 | 10.00 | A | O |
| ATOM | 93 | N | ILE | A | 26 | 8.770 | 21.402 | −6.679 | 1.00 | 10.00 | A | N |
| ATOM | 95 | CA | ILE | A | 26 | 9.250 | 22.660 | −7.247 | 1.00 | 10.00 | A | C |
| ATOM | 96 | CB | ILE | A | 26 | 9.171 | 23.803 | −6.202 | 1.00 | 10.00 | A | C |
| ATOM | 97 | CG1 | ILE | A | 26 | 9.309 | 25.181 | −6.860 | 1.00 | 10.00 | A | C |
| ATOM | 98 | CG2 | ILE | A | 26 | 10.206 | 23.618 | −5.102 | 1.00 | 10.00 | A | C |
| ATOM | 99 | CD1 | ILE | A | 26 | 9.032 | 26.342 | −5.924 | 1.00 | 10.00 | A | C |
| ATOM | 100 | C | ILE | A | 26 | 10.686 | 22.512 | −7.769 | 1.00 | 10.00 | A | C |
| ATOM | 101 | O | ILE | A | 26 | 11.384 | 21.563 | −7.413 | 1.00 | 10.00 | A | O |
| ATOM | 102 | N | THR | A | 27 | 11.109 | 23.445 | −8.619 | 1.00 | 10.00 | A | N |
| ATOM | 104 | CA | THR | A | 27 | 12.454 | 23.430 | −9.192 | 1.00 | 10.00 | A | C |
| ATOM | 105 | CB | THR | A | 27 | 12.708 | 24.660 | −10.105 | 1.00 | 10.00 | A | C |
| ATOM | 106 | OG1 | THR | A | 27 | 14.098 | 24.760 | −10.440 | 1.00 | 10.00 | A | O |
| ATOM | 108 | CG2 | THR | A | 27 | 12.253 | 25.949 | −9.439 | 1.00 | 10.00 | A | C |
| ATOM | 109 | C | THR | A | 27 | 13.541 | 23.331 | −8.116 | 1.00 | 10.00 | A | C |
| ATOM | 110 | O | THR | A | 27 | 14.510 | 22.594 | −8.277 | 1.00 | 10.00 | A | O |
| ATOM | 111 | N | SER | A | 28 | 13.361 | 24.058 | −7.016 | 1.00 | 10.00 | A | N |
| ATOM | 113 | CA | SER | A | 28 | 14.320 | 24.057 | −5.917 | 1.00 | 10.00 | A | C |
| ATOM | 114 | CB | SER | A | 28 | 13.876 | 25.064 | −4.861 | 1.00 | 10.00 | A | C |
| ATOM | 115 | OG | SER | A | 28 | 13.200 | 26.153 | −5.473 | 1.00 | 10.00 | A | O |
| ATOM | 117 | C | SER | A | 28 | 14.460 | 22.665 | −5.301 | 1.00 | 10.00 | A | C |
| ATOM | 118 | O | SER | A | 28 | 15.501 | 22.318 | −4.735 | 1.00 | 10.00 | A | O |
| ATOM | 119 | N | GLN | A | 29 | 13.417 | 21.864 | −5.440 | 1.00 | 10.00 | A | N |
| ATOM | 121 | CA | GLN | A | 29 | 13.411 | 20.513 | −4.909 | 1.00 | 10.00 | A | C |
| ATOM | 122 | CB | GLN | A | 29 | 11.977 | 20.075 | −4.588 | 1.00 | 10.00 | A | C |
| ATOM | 123 | CG | GLN | A | 29 | 11.443 | 20.578 | −3.256 | 1.00 | 10.00 | A | C |
| ATOM | 124 | CD | GLN | A | 29 | 9.984 | 20.230 | −3.052 | 1.00 | 10.00 | A | C |
| ATOM | 125 | OE1 | GLN | A | 29 | 9.194 | 20.231 | −3.996 | 1.00 | 10.00 | A | O |
| ATOM | 126 | NE2 | GLN | A | 29 | 9.617 | 19.921 | −1.821 | 1.00 | 10.00 | A | N |
| ATOM | 129 | C | GLN | A | 29 | 14.024 | 19.552 | −5.918 | 1.00 | 10.00 | A | C |
| ATOM | 130 | O | GLN | A | 29 | 14.758 | 18.630 | −5.557 | 1.00 | 10.00 | A | O |
| ATOM | 131 | N | LYS | A | 30 | 13.729 | 19.792 | −7.189 | 1.00 | 10.00 | A | N |
| ATOM | 133 | CA | LYS | A | 30 | 14.229 | 18.956 | −8.274 | 1.00 | 10.00 | A | C |
| ATOM | 134 | CB | LYS | A | 30 | 13.620 | 19.384 | −9.614 | 1.00 | 10.00 | A | C |
| ATOM | 135 | CG | LYS | A | 30 | 14.212 | 18.669 | −10.818 | 1.00 | 10.00 | A | C |
| ATOM | 136 | CD | LYS | A | 30 | 13.260 | 18.658 | −12.000 | 1.00 | 10.00 | A | C |
| ATOM | 137 | CE | LYS | A | 30 | 13.828 | 17.836 | −13.145 | 1.00 | 10.00 | A | C |
| ATOM | 138 | NZ | LYS | A | 30 | 14.140 | 16.445 | −12.727 | 1.00 | 10.00 | A | N |
| ATOM | 142 | C | LYS | A | 30 | 15.757 | 18.966 | −8.342 | 1.00 | 10.00 | A | C |
| ATOM | 143 | O | LYS | A | 30 | 16.378 | 17.960 | −8.689 | 1.00 | 10.00 | A | O |
| ATOM | 144 | N | SER | A | 31 | 16.362 | 20.095 | −7.983 | 1.00 | 10.00 | A | N |
| ATOM | 146 | CA | SER | A | 31 | 17.813 | 20.234 | −8.002 | 1.00 | 10.00 | A | C |
| ATOM | 147 | CB | SER | A | 31 | 18.215 | 21.637 | −7.546 | 1.00 | 10.00 | A | C |
| ATOM | 148 | OG | SER | A | 31 | 17.098 | 22.340 | −7.032 | 1.00 | 10.00 | A | O |
| ATOM | 150 | C | SER | A | 31 | 18.496 | 19.179 | −7.132 | 1.00 | 10.00 | A | C |
| ATOM | 151 | O | SER | A | 31 | 19.619 | 18.758 | −7.409 | 1.00 | 10.00 | A | O |
| ATOM | 152 | N | CYS | A | 32 | 17.801 | 18.739 | −6.093 | 1.00 | 10.00 | A | N |
| ATOM | 154 | CA | CYS | A | 32 | 18.338 | 17.745 | −5.180 | 1.00 | 10.00 | A | C |
| ATOM | 155 | CB | CYS | A | 32 | 17.749 | 17.958 | −3.788 | 1.00 | 10.00 | A | C |
| ATOM | 156 | SG | CYS | A | 32 | 17.720 | 19.688 | −3.262 | 1.00 | 10.00 | A | S |
| ATOM | 158 | C | CYS | A | 32 | 18.040 | 16.327 | −5.665 | 1.00 | 10.00 | A | C |
| ATOM | 159 | O | CYS | A | 32 | 18.756 | 15.383 | −5.328 | 1.00 | 10.00 | A | O |
| ATOM | 160 | N | GLU | A | 33 | 16.985 | 16.188 | −6.460 | 1.00 | 10.00 | A | N |
| ATOM | 162 | CA | GLU | A | 33 | 16.583 | 14.889 | −6.987 | 1.00 | 10.00 | A | C |
| ATOM | 163 | CB | GLU | A | 33 | 15.146 | 14.954 | −7.534 | 1.00 | 10.00 | A | C |
| ATOM | 164 | CG | GLU | A | 33 | 14.892 | 14.193 | −8.827 | 1.00 | 10.00 | A | C |
| ATOM | 165 | CD | GLU | A | 33 | 14.600 | 15.124 | −9.988 | 1.00 | 10.00 | A | C |
| ATOM | 166 | OE1 | GLU | A | 33 | 13.424 | 15.264 | −10.363 | 1.00 | 10.00 | A | O |
| ATOM | 167 | OE2 | GLU | A | 33 | 15.548 | 15.729 | −10.529 | 1.00 | 10.00 | A | O |
| ATOM | 168 | C | GLU | A | 33 | 17.585 | 14.383 | −8.030 | 1.00 | 10.00 | A | C |
| ATOM | 169 | O | GLU | A | 33 | 18.122 | 13.277 | −7.906 | 1.00 | 10.00 | A | O |
| ATOM | 170 | N | CYS | A | 34 | 17.856 | 15.206 | −9.036 | 1.00 | 10.00 | A | N |
| ATOM | 172 | CA | CYS | A | 34 | 18.797 | 14.844 | −10.088 | 1.00 | 10.00 | A | C |
| ATOM | 173 | CB | CYS | A | 34 | 18.781 | 15.894 | −11.200 | 1.00 | 10.00 | A | C |
| ATOM | 174 | SG | CYS | A | 34 | 18.594 | 17.596 | −10.613 | 1.00 | 10.00 | A | s |
| ATOM | 176 | C | CYS | A | 34 | 20.206 | 14.685 | −9.526 | 1.00 | 10.00 | A | C |
| ATOM | 177 | O | CYS | A | 34 | 20.973 | 13.832 | −9.975 | 1.00 | 10.00 | A | O |
| ATOM | 178 | N | LEU | A | 35 | 20.525 | 15.503 | −8.526 | 1.00 | 10.00 | A | N |
| ATOM | 180 | CA | LEU | A | 35 | 21.833 | 15.463 | −7.888 | 1.00 | 10.00 | A | C |
| ATOM | 181 | CB | LEU | A | 35 | 21.972 | 16.613 | −6.884 | 1.00 | 10.00 | A | C |
| ATOM | 182 | CG | LEU | A | 35 | 23.352 | 16.805 | −6.246 | 1.00 | 10.00 | A | C |
| ATOM | 183 | CD1 | LEU | A | 35 | 24.372 | 17.233 | −7.289 | 1.00 | 10.00 | A | C |
| ATOM | 184 | CD2 | LEU | A | 35 | 23.284 | 17.822 | −5.117 | 1.00 | 10.00 | A | C |
| ATOM | 185 | C | LEU | A | 35 | 22.038 | 14.128 | −7.185 | 1.00 | 10.00 | A | C |
| ATOM | 186 | O | LEU | A | 35 | 23.160 | 13.641 | −7.065 | 1.00 | 10.00 | A | O |
| ATOM | 187 | N | ALA | A | 36 | 20.943 | 13.533 | −6.733 | 1.00 | 10.00 | A | N |
| ATOM | 189 | CA | ALA | A | 36 | 21.001 | 12.256 | −6.047 | 1.00 | 10.00 | A | C |
| ATOM | 190 | CB | ALA | A | 36 | 19.752 | 12.041 | −5.205 | 1.00 | 10.00 | A | C |
| ATOM | 191 | C | ALA | A | 36 | 21.172 | 11.124 | −7.048 | 1.00 | 10.00 | A | C |
| ATOM | 192 | O | ALA | A | 36 | 22.026 | 10.255 | −6.873 | 1.00 | 10.00 | A | O |
| ATOM | 193 | N | GLU | A | 37 | 20.365 | 11.151 | −8.105 | 1.00 | 10.00 | A | N |
| ATOM | 195 | CA | GLU | A | 37 | 20.423 | 10.121 | −9.138 | 1.00 | 10.00 | A | C |
| ATOM | 196 | CB | GLU | A | 37 | 19.310 | 10.323 | −10.167 | 1.00 | 10.00 | A | C |
| ATOM | 197 | CG | GLU | A | 37 | 19.097 | 9.131 | −11.086 | 1.00 | 10.00 | A | C |
| ATOM | 198 | CD | GLU | A | 37 | 19.231 | 9.485 | −12.553 | 1.00 | 10.00 | A | C |
| ATOM | 199 | OE1 | GLU | A | 37 | 20.377 | 9.610 | −13.041 | 1.00 | 10.00 | A | O |
| ATOM | 200 | OE2 | GLU | A | 37 | 18.196 | 9.633 | −13.227 | 1.00 | 10.00 | A | O |
| ATOM | 201 | C | GLU | A | 37 | 21.778 | 10.114 | −9.830 | 1.00 | 10.00 | A | C |
| ATOM | 202 | O | GLU | A | 37 | 22.400 | 9.065 | −9.990 | 1.00 | 10.00 | A | O |
| ATOM | 203 | N | THR | A | 38 | 22.237 | 11.292 | −10.226 | 1.00 | 10.00 | A | N |
| ATOM | 205 | CA | THR | A | 38 | 23.520 | 11.418 | −10.896 | 1.00 | 10.00 | A | C |
| ATOM | 206 | CB | THR | A | 38 | 23.739 | 12.837 | −11.480 | 1.00 | 10.00 | A | C |
| ATOM | 207 | OG1 | THR | A | 38 | 24.640 | 12.772 | −12.590 | 1.00 | 10.00 | A | O |
| ATOM | 209 | CG2 | THR | A | 38 | 24.277 | 13.809 | −10.440 | 1.00 | 10.00 | A | C |
| ATOM | 210 | C | THR | A | 38 | 24.673 | 11.003 | −9.980 | 1.00 | 10.00 | A | C |
| ATOM | 211 | O | THR | A | 38 | 25.679 | 10.461 | −10.442 | 1.00 | 10.00 | A | O |
| ATOM | 212 | N | TRP | A | 39 | 24.506 | 11.227 | −8.680 | 1.00 | 10.00 | A | N |
| ATOM | 214 | CA | TRP | A | 39 | 25.530 | 10.870 | −7.710 | 1.00 | 10.00 | A | C |
| ATOM | 215 | CB | TRP | A | 39 | 25.171 | 11.422 | −6.329 | 1.00 | 10.00 | A | C |
| ATOM | 216 | CG | TRP | A | 39 | 26.324 | 11.468 | −5.371 | 1.00 | 10.00 | A | C |
| ATOM | 217 | CD1 | TRP | A | 39 | 26.433 | 10.798 | −4.188 | 1.00 | 10.00 | A | C |
| ATOM | 218 | CD2 | TRP | A | 39 | 27.530 | 12.229 | −5.516 | 1.00 | 10.00 | A | C |
| ATOM | 219 | NE1 | TRP | A | 39 | 27.632 | 11.092 | −3.586 | 1.00 | 10.00 | A | N |
| ATOM | 221 | CE2 | TRP | A | 39 | 28.326 | 11.967 | −4.382 | 1.00 | 10.00 | A | C |
| ATOM | 222 | CE3 | TRP | A | 39 | 28.015 | 13.105 | −6.496 | 1.00 | 10.00 | A | C |
| ATOM | 223 | CZ2 | TRP | A | 39 | 29.580 | 12.547 | −4.201 | 1.00 | 10.00 | A | C |
| ATOM | 224 | CZ3 | TRP | A | 39 | 29.259 | 13.680 | −6.314 | 1.00 | 10.00 | A | C |
| ATOM | 225 | CH2 | TRP | A | 39 | 30.029 | 13.397 | −5.177 | 1.00 | 10.00 | A | C |
| ATOM | 226 | C | TRP | A | 39 | 25.672 | 9.358 | −7.648 | 1.00 | 10.00 | A | C |
| ATOM | 227 | O | TRP | A | 39 | 26.780 | 8.824 | −7.719 | 1.00 | 10.00 | A | O |
| ATOM | 228 | N | ALA | A | 40 | 24.538 | 8.676 | −7.536 | 1.00 | 10.00 | A | N |
| ATOM | 230 | CA | ALA | A | 40 | 24.524 | 7.222 | −7.468 | 1.00 | 10.00 | A | C |
| ATOM | 231 | CB | ALA | A | 40 | 23.125 | 6.721 | −7.141 | 1.00 | 10.00 | A | C |
| ATOM | 232 | C | ALA | A | 40 | 25.023 | 6.612 | −8.773 | 1.00 | 10.00 | A | C |
| ATOM | 233 | O | ALA | A | 40 | 25.848 | 5.704 | −8.764 | 1.00 | 10.00 | A | O |
| ATOM | 234 | N | TYR | A | 41 | 24.538 | 7.139 | −9.895 | 1.00 | 10.00 | A | N |
| ATOM | 236 | CA | TYR | A | 41 | 24.927 | 6.645 | −11.215 | 1.00 | 10.00 | A | C |
| ATOM | 237 | CB | TYR | A | 41 | 24.182 | 7.406 | −12.319 | 1.00 | 10.00 | A | C |
| ATOM | 238 | CG | TYR | A | 41 | 24.492 | 6.916 | −13.717 | 1.00 | 10.00 | A | C |
| ATOM | 239 | CD1 | TYR | A | 41 | 24.173 | 5.623 | −14.107 | 1.00 | 10.00 | A | C |
| ATOM | 240 | CD2 | TYR | A | 41 | 25.113 | 7.744 | −14.641 | 1.00 | 10.00 | A | C |
| ATOM | 241 | CE1 | TYR | A | 41 | 24.466 | 5.166 | −15.377 | 1.00 | 10.00 | A | C |
| ATOM | 242 | CE2 | TYR | A | 41 | 25.406 | 7.300 | −15.915 | 1.00 | 10.00 | A | C |
| ATOM | 243 | CZ | TYR | A | 41 | 25.081 | 6.012 | −16.278 | 1.00 | 10.00 | A | C |
| ATOM | 244 | OH | TYR | A | 41 | 25.382 | 5.560 | −17.543 | 1.00 | 10.00 | A | O |
| ATOM | 246 | C | TYR | A | 41 | 26.438 | 6.737 | −11.432 | 1.00 | 10.00 | A | C |
| ATOM | 247 | O | TYR | A | 41 | 27.042 | 5.855 | −12.045 | 1.00 | 10.00 | A | O |
| ATOM | 248 | N | CYS | A | 42 | 27.041 | 7.805 | −10.929 | 1.00 | 10.00 | A | N |
| ATOM | 250 | CA | CYS | A | 42 | 28.479 | 8.001 | −11.069 | 1.00 | 10.00 | A | C |
| ATOM | 251 | CB | CYS | A | 42 | 28.862 | 9.449 | −10.760 | 1.00 | 10.00 | A | C |
| ATOM | 252 | SG | CYS | A | 42 | 28.213 | 10.658 | −11.939 | 1.00 | 10.00 | A | S |
| ATOM | 254 | C | CYS | A | 42 | 29.265 | 7.036 | −10.182 | 1.00 | 10.00 | A | C |
| ATOM | 255 | O | CYS | A | 42 | 30.330 | 6.557 | −10.567 | 1.00 | 10.00 | A | O |
| ATOM | 256 | N | LEU | A | 43 | 28.730 | 6.743 | −9.002 | 1.00 | 10.00 | A | N |
| ATOM | 258 | CA | LEU | A | 43 | 29.392 | 5.843 | −8.070 | 1.00 | 10.00 | A | C |
| ATOM | 259 | CB | LEU | A | 43 | 28.933 | 6.117 | −6.636 | 1.00 | 10.00 | A | C |
| ATOM | 260 | CG | LEU | A | 43 | 29.260 | 7.510 | −6.097 | 1.00 | 10.00 | A | C |
| ATOM | 261 | CD1 | LEU | A | 43 | 28.447 | 7.809 | −4.852 | 1.00 | 10.00 | A | C |
| ATOM | 262 | CD2 | LEU | A | 43 | 30.749 | 7.643 | −5.817 | 1.00 | 10.00 | A | C |
| ATOM | 263 | C | LEU | A | 43 | 29.177 | 4.381 | −8.447 | 1.00 | 10.00 | A | C |
| ATOM | 264 | O | LEU | A | 43 | 30.113 | 3.586 | −8.435 | 1.00 | 10.00 | A | O |
| ATOM | 265 | N | GLN | A | 44 | 27.954 | 4.036 | −8.821 | 1.00 | 10.00 | A | N |
| ATOM | 267 | CA | GLN | A | 44 | 27.626 | 2.662 | −9.199 | 1.00 | 10.00 | A | C |
| ATOM | 268 | CB | GLN | A | 44 | 26.182 | 2.343 | −8.822 | 1.00 | 10.00 | A | C |
| ATOM | 269 | CG | GLN | A | 44 | 25.750 | 2.846 | −7.459 | 1.00 | 10.00 | A | C |
| ATOM | 270 | CD | GLN | A | 44 | 24.245 | 3.002 | −7.373 | 1.00 | 10.00 | A | C |
| ATOM | 271 | OE1 | GLN | A | 44 | 23.580 | 3.234 | −8.379 | 1.00 | 10.00 | A | O |
| ATOM | 272 | NE2 | GLN | A | 44 | 23.697 | 2.877 | −6.174 | 1.00 | 10.00 | A | N |
| ATOM | 275 | C | GLN | A | 44 | 27.796 | 2.452 | −10.702 | 1.00 | 10.00 | A | C |
| ATOM | 276 | O | GLN | A | 44 | 26.968 | 1.809 | −11.350 | 1.00 | 10.00 | A | O |
| ATOM | 277 | N | GLN | A | 45 | 28.874 | 2.974 | −11.254 | 1.00 | 10.00 | A | N |
| ATOM | 279 | CA | GLN | A | 45 | 29.123 | 2.847 | −12.680 | 1.00 | 10.00 | A | C |
| ATOM | 280 | CB | GLN | A | 45 | 29.994 | 3.996 | −13.195 | 1.00 | 10.00 | A | C |
| ATOM | 281 | CG | GLN | A | 45 | 30.189 | 3.988 | −14.706 | 1.00 | 10.00 | A | C |
| ATOM | 282 | CD | GLN | A | 45 | 28.887 | 4.121 | −15.475 | 1.00 | 10.00 | A | C |
| ATOM | 283 | OE1 | GLN | A | 45 | 28.703 | 3.497 | −16.519 | 1.00 | 10.00 | A | O |
| ATOM | 284 | NE2 | GLN | A | 45 | 27.978 | 4.948 | −14.979 | 1.00 | 10.00 | A | N |
| ATOM | 287 | C | GLN | A | 45 | 29.747 | 1.501 | −13.036 | 1.00 | 10.00 | A | C |
| ATOM | 288 | O | GLN | A | 45 | 29.183 | 0.730 | −13.813 | 1.00 | 10.00 | A | O |
| ATOM | 289 | N | THR | A | 46 | 30.908 | 1.219 | −12.466 | 1.00 | 10.00 | A | N |
| ATOM | 291 | CA | THR | A | 46 | 31.609 | −0.029 | −12.737 | 1.00 | 10.00 | A | C |
| ATOM | 292 | CB | THR | A | 46 | 33.126 | 0.201 | −12.766 | 1.00 | 10.00 | A | C |
| ATOM | 293 | OG1 | THR | A | 46 | 33.476 | 1.130 | −11.734 | 1.00 | 10.00 | A | O |
| ATOM | 295 | CG2 | THR | A | 46 | 33.546 | 0.771 | −14.111 | 1.00 | 10.00 | A | C |
| ATOM | 296 | C | THR | A | 46 | 31.270 | −1.115 | −11.716 | 1.00 | 10.00 | A | C |
| ATOM | 297 | O | THR | A | 46 | 31.990 | −2.106 | −11.585 | 1.00 | 10.00 | A | O |
| ATOM | 298 | N | GLN | A | 47 | 30.166 | −0.933 | −11.009 | 1.00 | 10.00 | A | N |
| ATOM | 300 | CA | GLN | A | 47 | 29.729 | −1.894 | −10.011 | 1.00 | 10.00 | A | C |
| ATOM | 301 | CB | GLN | A | 47 | 29.852 | −1.297 | −8.605 | 1.00 | 10.00 | A | C |
| ATOM | 302 | CG | GLN | A | 47 | 29.644 | −2.295 | −7.474 | 1.00 | 10.00 | A | C |
| ATOM | 303 | CD | GLN | A | 47 | 30.577 | −3.489 | −7.567 | 1.00 | 10.00 | A | C |
| ATOM | 304 | OE1 | GLN | A | 47 | 31.684 | −3.391 | −8.099 | 1.00 | 10.00 | A | O |
| ATOM | 305 | NE2 | GLN | A | 47 | 30.142 | −4.621 | −7.040 | 1.00 | 10.00 | A | N |
| ATOM | 308 | C | GLN | A | 47 | 28.289 | −2.302 | −10.293 | 1.00 | 10.00 | A | C |
| ATOM | 309 | O | GLN | A | 47 | 27.394 | −1.457 | −10.322 | 1.00 | 10.00 | A | O |
| ATOM | 310 | N | GLY | A | 48 | 28.077 | −3.587 | −10.534 | 1.00 | 10.00 | A | N |
| ATOM | 312 | CA | GLY | A | 48 | 26.747 | −4.080 | −10.819 | 1.00 | 10.00 | A | C |
| ATOM | 313 | C | GLY | A | 48 | 26.331 | −5.184 | −9.871 | 1.00 | 10.00 | A | C |
| ATOM | 314 | O | GLY | A | 48 | 27.167 | −5.751 | −9.167 | 1.00 | 10.00 | A | O |
| ATOM | 315 | N | HIS | A | 49 | 25.043 | −5.493 | −9.862 | 1.00 | 10.00 | A | N |
| ATOM | 317 | CA | HIS | A | 49 | 24.502 | −6.538 | −9.008 | 1.00 | 10.00 | A | C |
| ATOM | 318 | CB | HIS | A | 49 | 23.067 | −6.182 | −8.602 | 1.00 | 10.00 | A | C |
| ATOM | 319 | CG | HIS | A | 49 | 22.905 | −5.747 | −7.179 | 1.00 | 10.00 | A | C |
| ATOM | 320 | ND1 | HIS | A | 49 | 21.789 | −5.055 | −6.768 | 1.00 | 10.00 | A | N |
| ATOM | 321 | CD2 | HIS | A | 49 | 23.721 | −5.948 | −6.115 | 1.00 | 10.00 | A | C |
| ATOM | 322 | CE1 | HIS | A | 49 | 21.947 | −4.854 | −5.472 | 1.00 | 10.00 | A | C |
| ATOM | 323 | NE2 | HIS | A | 49 | 23.097 | −5.376 | −5.036 | 1.00 | 10.00 | A | N |
| ATOM | 325 | C | HIS | A | 49 | 24.504 | −7.862 | −9.763 | 1.00 | 10.00 | A | C |
| ATOM | 326 | O | HIS | A | 49 | 24.526 | −7.869 | −11.001 | 1.00 | 10.00 | A | O |
| ATOM | 327 | N | PRO | A | 50 | 24.497 | −8.995 | −9.035 | 1.00 | 10.00 | A | N |
| ATOM | 328 | CA | PRO | A | 50 | 24.484 | −10.334 | −9.637 | 1.00 | 10.00 | A | C |
| ATOM | 329 | CB | PRO | A | 50 | 24.307 | −11.262 | −8.429 | 1.00 | 10.00 | A | C |
| ATOM | 330 | CG | PRO | A | 50 | 24.870 | −10.492 | −7.287 | 1.00 | 10.00 | A | C |
| ATOM | 331 | CD | PRO | A | 50 | 24.521 | −9.058 | −7.560 | 1.00 | 10.00 | A | C |
| ATOM | 332 | C | PRO | A | 50 | 23.313 | −10.509 | −10.600 | 1.00 | 10.00 | A | C |
| ATOM | 333 | O | PRO | A | 50 | 22.349 | −9.742 | −10.565 | 1.00 | 10.00 | A | O |
| ATOM | 334 | N | ASP | A | 51 | 23.412 | −11.516 | −11.464 | 1.00 | 10.00 | A | N |
| ATOM | 336 | CA | ASP | A | 51 | 22.370 | −11.811 | −12.451 | 1.00 | 10.00 | A | C |
| ATOM | 337 | CB | ASP | A | 51 | 20.992 | −12.002 | −11.792 | 1.00 | 10.00 | A | C |
| ATOM | 338 | CG | ASP | A | 51 | 20.995 | −13.060 | −10.711 | 1.00 | 10.00 | A | C |
| ATOM | 339 | OD1 | ASP | A | 51 | 21.301 | −14.226 | −11.020 | 1.00 | 10.00 | A | O |
| ATOM | 340 | OD2 | ASP | A | 51 | 20.686 | −12.727 | −9.547 | 1.00 | 10.00 | A | O |
| ATOM | 341 | C | ASP | A | 51 | 22.311 | −10.731 | −13.524 | 1.00 | 10.00 | A | C |
| ATOM | 342 | O | ASP | A | 51 | 21.313 | −10.591 | −14.230 | 1.00 | 10.00 | A | O |
| ATOM | 343 | N | ALA | A | 52 | 23.408 | −9.984 | −13.643 | 1.00 | 10.00 | A | N |
| ATOM | 345 | CA | ALA | A | 52 | 23.534 | −8.905 | −14.621 | 1.00 | 10.00 | A | C |
| ATOM | 346 | CB | ALA | A | 52 | 23.535 | −9.443 | −16.044 | 1.00 | 10.00 | A | C |
| ATOM | 347 | C | ALA | A | 52 | 22.459 | −7.836 | −14.437 | 1.00 | 10.00 | A | C |
| ATOM | 348 | O | ALA | A | 52 | 21.748 | −7.489 | −15.384 | 1.00 | 10.00 | A | O |
| ATOM | 349 | N | SER | A | 53 | 22.343 | −7.315 | −13.223 | 1.00 | 10.00 | A | N |
| ATOM | 351 | CA | SER | A | 53 | 21.364 | −6.283 | −12.936 | 1.00 | 10.00 | A | C |
| ATOM | 352 | CB | SER | A | 53 | 20.399 | −6.730 | −11.835 | 1.00 | 10.00 | A | C |
| ATOM | 353 | OG | SER | A | 53 | 21.090 | −7.337 | −10.754 | 1.00 | 10.00 | A | O |
| ATOM | 355 | C | SER | A | 53 | 22.051 | −4.962 | −12.580 | 1.00 | 10.00 | A | C |
| ATOM | 356 | O | SER | A | 53 | 23.049 | −4.939 | −11.849 | 1.00 | 10.00 | A | O |
| ATOM | 357 | N | LEU | A | 54 | 21.535 | −3.869 | −13.131 | 1.00 | 10.00 | A | N |
| ATOM | 359 | CA | LEU | A | 54 | 22.090 | −2.546 | −12.882 | 1.00 | 10.00 | A | C |
| ATOM | 360 | CB | LEU | A | 54 | 21.419 | −1.513 | −13.796 | 1.00 | 10.00 | A | C |
| ATOM | 361 | CG | LEU | A | 54 | 22.316 | −0.825 | −14.833 | 1.00 | 10.00 | A | C |
| ATOM | 362 | CD1 | LEU | A | 54 | 23.131 | 0.286 | −14.190 | 1.00 | 10.00 | A | C |
| ATOM | 363 | CD2 | LEU | A | 54 | 23.232 | −1.831 | −15.516 | 1.00 | 10.00 | A | C |
| ATOM | 364 | C | LEU | A | 54 | 21.906 | −2.157 | −11.421 | 1.00 | 10.00 | A | C |
| ATOM | 365 | O | LEU | A | 54 | 20.818 | −2.300 | −10.869 | 1.00 | 10.00 | A | O |
| ATOM | 366 | N | LEU | A | 55 | 22.964 | −1.652 | −10.805 | 1.00 | 10.00 | A | N |
| ATOM | 368 | CA | LEU | A | 55 | 22.914 | −1.256 | −9.403 | 1.00 | 10.00 | A | C |
| ATOM | 369 | CB | LEU | A | 55 | 24.316 | −0.926 | −8.884 | 1.00 | 10.00 | A | C |
| ATOM | 370 | CG | LEU | A | 55 | 24.494 | −0.887 | −7.362 | 1.00 | 10.00 | A | C |
| ATOM | 371 | CD1 | LEU | A | 55 | 23.878 | −2.115 | −6.713 | 1.00 | 10.00 | A | C |
| ATOM | 372 | CD2 | LEU | A | 55 | 25.967 | −0.783 | −6.998 | 1.00 | 10.00 | A | C |
| ATOM | 373 | C | LEU | A | 55 | 21.955 | −0.089 | −9.182 | 1.00 | 10.00 | A | C |
| ATOM | 374 | O | LEU | A | 55 | 21.265 | −0.029 | −8.168 | 1.00 | 10.00 | A | O |
| ATOM | 375 | N | LEU | A | 56 | 21.907 | 0.825 | −10.140 | 1.00 | 10.00 | A | N |
| ATOM | 377 | CA | LEU | A | 56 | 21.023 | 1.977 | −10.042 | 1.00 | 10.00 | A | C |
| ATOM | 378 | CB | LEU | A | 56 | 21.470 | 3.088 | −10.998 | 1.00 | 10.00 | A | C |
| ATOM | 379 | CG | LEU | A | 56 | 20.673 | 4.394 | −10.947 | 1.00 | 10.00 | A | C |
| ATOM | 380 | CD1 | LEU | A | 56 | 20.888 | 5.102 | −9.620 | 1.00 | 10.00 | A | C |
| ATOM | 381 | CD2 | LEU | A | 56 | 21.049 | 5.301 | −12.107 | 1.00 | 10.00 | A | C |
| ATOM | 382 | C | LEU | A | 56 | 19.581 | 1.580 | −10.337 | 1.00 | 10.00 | A | C |
| ATOM | 383 | O | LEU | A | 56 | 18.648 | 2.228 | −9.871 | 1.00 | 10.00 | A | O |
| ATOM | 384 | N | TRP | A | 57 | 19.407 | 0.501 | −11.092 | 1.00 | 10.00 | A | N |
| ATOM | 386 | CA | TRP | A | 57 | 18.078 | 0.023 | −11.460 | 1.00 | 10.00 | A | C |
| ATOM | 387 | CB | TRP | A | 57 | 18.168 | −1.248 | −12.308 | 1.00 | 10.00 | A | C |
| ATOM | 388 | CG | TRP | A | 57 | 16.876 | −1.995 | −12.416 | 1.00 | 10.00 | A | C |
| ATOM | 389 | CD1 | TRP | A | 57 | 15.730 | −1.571 | −13.022 | 1.00 | 10.00 | A | C |
| ATOM | 390 | CD2 | TRP | A | 57 | 16.598 | −3.299 | −11.898 | 1.00 | 10.00 | A | C |
| ATOM | 391 | NE1 | TRP | A | 57 | 14.753 | −2.526 | −12.903 | 1.00 | 10.00 | A | N |
| ATOM | 393 | CE2 | TRP | A | 57 | 15.261 | −3.598 | −12.218 | 1.00 | 10.00 | A | C |
| ATOM | 394 | CE3 | TRP | A | 57 | 17.351 | −4.240 | −11.191 | 1.00 | 10.00 | A | C |
| ATOM | 395 | CZ2 | TRP | A | 57 | 14.658 | −4.799 | −11.857 | 1.00 | 10.00 | A | C |
| ATOM | 396 | CZ3 | TRP | A | 57 | 16.751 | −5.433 | −10.835 | 1.00 | 10.00 | A | C |
| ATOM | 397 | CH2 | TRP | A | 57 | 15.419 | −5.704 | −11.168 | 1.00 | 10.00 | A | C |
| ATOM | 398 | C | TRP | A | 57 | 17.191 | −0.192 | −10.232 | 1.00 | 10.00 | A | C |
| ATOM | 399 | O | TRP | A | 57 | 16.089 | 0.352 | −10.158 | 1.00 | 10.00 | A | O |
| ATOM | 400 | N | ARG | A | 58 | 17.682 | −0.963 | −9.263 | 1.00 | 10.00 | A | N |
| ATOM | 402 | CA | ARG | A | 58 | 16.912 | −1.233 | −8.051 | 1.00 | 10.00 | A | C |
| ATOM | 403 | CB | ARG | A | 58 | 17.557 | −2.321 | −7.192 | 1.00 | 10.00 | A | C |
| ATOM | 404 | CG | ARG | A | 58 | 17.093 | −3.721 | −7.556 | 1.00 | 10.00 | A | C |
| ATOM | 405 | CD | ARG | A | 58 | 17.882 | −4.791 | −6.826 | 1.00 | 10.00 | A | C |
| ATOM | 406 | NE | ARG | A | 58 | 17.538 | −6.127 | −7.305 | 1.00 | 10.00 | A | N |
| ATOM | 408 | CZ | ARG | A | 58 | 18.317 | −6.856 | −8.105 | 1.00 | 10.00 | A | C |
| ATOM | 409 | NH1 | ARG | A | 58 | 19.483 | −6.376 | −8.524 | 1.00 | 10.00 | A | N |
| ATOM | 412 | NH2 | ARG | A | 58 | 17.938 | −8.068 | −8.463 | 1.00 | 10.00 | A | N |
| ATOM | 415 | C | ARG | A | 58 | 16.681 | 0.041 | −7.259 | 1.00 | 10.00 | A | C |
| ATOM | 416 | O | ARG | A | 58 | 15.627 | 0.212 | −6.652 | 1.00 | 10.00 | A | O |
| ATOM | 417 | N | GLY | A | 59 | 17.664 | 0.936 | −7.285 | 1.00 | 10.00 | A | N |
| ATOM | 419 | CA | GLY | A | 59 | 17.533 | 2.198 | −6.588 | 1.00 | 10.00 | A | C |
| ATOM | 420 | C | GLY | A | 59 | 16.433 | 3.040 | −7.201 | 1.00 | 10.00 | A | C |
| ATOM | 421 | O | GLY | A | 59 | 15.597 | 3.598 | −6.492 | 1.00 | 10.00 | A | O |
| ATOM | 422 | N | VAL | A | 60 | 16.416 | 3.103 | −8.531 | 1.00 | 10.00 | A | N |
| ATOM | 424 | CA | VAL | A | 60 | 15.407 | 3.865 | −9.257 | 1.00 | 10.00 | A | C |
| ATOM | 425 | CB | VAL | A | 60 | 15.672 | 3.859 | −10.781 | 1.00 | 10.00 | A | C |
| ATOM | 426 | CG1 | VAL | A | 60 | 14.506 | 4.468 | −11.552 | 1.00 | 10.00 | A | C |
| ATOM | 427 | CG2 | VAL | A | 60 | 16.957 | 4.609 | −11.094 | 1.00 | 10.00 | A | C |
| ATOM | 428 | C | VAL | A | 60 | 14.017 | 3.311 | −8.963 | 1.00 | 10.00 | A | C |
| ATOM | 429 | O | VAL | A | 60 | 13.064 | 4.071 | −8.792 | 1.00 | 10.00 | A | O |
| ATOM | 430 | N | GLU | A | 61 | 13.916 | 1.985 | −8.885 | 1.00 | 10.00 | A | N |
| ATOM | 432 | CA | GLU | A | 61 | 12.647 | 1.325 | −8.590 | 1.00 | 10.00 | A | C |
| ATOM | 433 | CB | GLU | A | 61 | 12.836 | −0.194 | −8.538 | 1.00 | 10.00 | A | C |
| ATOM | 434 | CG | GLU | A | 61 | 12.877 | −0.868 | −9.899 | 1.00 | 10.00 | A | C |
| ATOM | 435 | CD | GLU | A | 61 | 11.507 | −0.966 | −10.536 | 1.00 | 10.00 | A | C |
| ATOM | 436 | OE1 | GLU | A | 61 | 11.206 | −0.161 | −11.441 | 1.00 | 10.00 | A | O |
| ATOM | 437 | OE2 | GLU | A | 61 | 10.724 | −1.852 | −10.136 | 1.00 | 10.00 | A | O |
| ATOM | 438 | C | GLU | A | 61 | 12.078 | 1.824 | −7.265 | 1.00 | 10.00 | A | C |
| ATOM | 439 | O | GLU | A | 61 | 10.882 | 2.084 | −7.143 | 1.00 | 10.00 | A | O |
| ATOM | 440 | N | MET | A | 62 | 12.953 | 1.979 | −6.280 | 1.00 | 10.00 | A | N |
| ATOM | 442 | CA | MET | A | 62 | 12.543 | 2.447 | −4.963 | 1.00 | 10.00 | A | C |
| ATOM | 443 | CB | MET | A | 62 | 13.692 | 2.302 | −3.964 | 1.00 | 10.00 | A | C |
| ATOM | 444 | CG | MET | A | 62 | 14.303 | 0.912 | −3.900 | 1.00 | 10.00 | A | C |
| ATOM | 445 | SD | MET | A | 62 | 15.865 | 0.878 | −2.995 | 1.00 | 10.00 | A | S |
| ATOM | 446 | CE | MET | A | 62 | 16.495 | −0.735 | −3.464 | 1.00 | 10.00 | A | C |
| ATOM | 447 | C | MET | A | 62 | 12.099 | 3.906 | −5.029 | 1.00 | 10.00 | A | C |
| ATOM | 448 | O | MET | A | 62 | 11.056 | 4.276 | −4.493 | 1.00 | 10.00 | A | O |
| ATOM | 449 | N | VAL | A | 63 | 12.895 | 4.723 | −5.713 | 1.00 | 10.00 | A | N |
| ATOM | 451 | CA | VAL | A | 63 | 12.608 | 6.149 | −5.859 | 1.00 | 10.00 | A | C |
| ATOM | 452 | CB | VAL | A | 63 | 13.743 | 6.878 | −6.607 | 1.00 | 10.00 | A | C |
| ATOM | 453 | CG1 | VAL | A | 63 | 13.513 | 8.382 | −6.619 | 1.00 | 10.00 | A | C |
| ATOM | 454 | CG2 | VAL | A | 63 | 15.079 | 6.558 | −5.971 | 1.00 | 10.00 | A | C |
| ATOM | 455 | C | VAL | A | 63 | 11.299 | 6.374 | −6.606 | 1.00 | 10.00 | A | C |
| ATOM | 456 | O | VAL | A | 63 | 10.525 | 7.272 | −6.265 | 1.00 | 10.00 | A | O |
| ATOM | 457 | N | GLU | A | 64 | 11.055 | 5.549 | −7.614 | 1.00 | 10.00 | A | N |
| ATOM | 459 | CA | GLU | A | 64 | 9.841 | 5.649 | −8.411 | 1.00 | 10.00 | A | C |
| ATOM | 460 | CB | GLU | A | 64 | 9.816 | 4.576 | −9.501 | 1.00 | 10.00 | A | C |
| ATOM | 461 | CG | GLU | A | 64 | 8.749 | 4.801 | −10.561 | 1.00 | 10.00 | A | C |
| ATOM | 462 | CD | GLU | A | 64 | 8.817 | 3.781 | −11.673 | 1.00 | 10.00 | A | C |
| ATOM | 463 | OE1 | GLU | A | 64 | 8.085 | 2.772 | −11.607 | 1.00 | 10.00 | A | O |
| ATOM | 464 | OE2 | GLU | A | 64 | 9.597 | 3.981 | −12.623 | 1.00 | 10.00 | A | O |
| ATOM | 465 | C | GLU | A | 64 | 8.606 | 5.544 | −7.523 | 1.00 | 10.00 | A | C |
| ATOM | 466 | O | GLU | A | 64 | 7.698 | 6.368 | −7.612 | 1.00 | 10.00 | A | O |
| ATOM | 467 | N | ILE | A | 65 | 8.601 | 4.549 | −6.640 | 1.00 | 10.00 | A | N |
| ATOM | 469 | CA | ILE | A | 65 | 7.487 | 4.338 | −5.722 | 1.00 | 10.00 | A | C |
| ATOM | 470 | CB | ILE | A | 65 | 7.720 | 3.089 | −4.844 | 1.00 | 10.00 | A | C |
| ATOM | 471 | CG1 | ILE | A | 65 | 7.939 | 1.858 | −5.724 | 1.00 | 10.00 | A | C |
| ATOM | 472 | CG2 | ILE | A | 65 | 6.548 | 2.865 | −3.900 | 1.00 | 10.00 | A | C |
| ATOM | 473 | CD1 | ILE | A | 65 | 8.577 | 0.696 | −4.998 | 1.00 | 10.00 | A | C |
| ATOM | 474 | C | ILE | A | 65 | 7.283 | 5.568 | −4.829 | 1.00 | 10.00 | A | C |
| ATOM | 475 | O | ILE | A | 65 | 6.153 | 5.982 | −4.567 | 1.00 | 10.00 | A | O |
| ATOM | 476 | N | LEU | A | 66 | 8.391 | 6.157 | −4.381 | 1.00 | 10.00 | A | N |
| ATOM | 478 | CA | LEU | A | 66 | 8.340 | 7.346 | −3.533 | 1.00 | 10.00 | A | C |
| ATOM | 479 | CB | LEU | A | 66 | 9.729 | 7.685 | −2.988 | 1.00 | 10.00 | A | C |
| ATOM | 480 | CG | LEU | A | 66 | 10.355 | 6.657 | −2.047 | 1.00 | 10.00 | A | C |
| ATOM | 481 | CD1 | LEU | A | 66 | 11.792 | 7.033 | −1.735 | 1.00 | 10.00 | A | C |
| ATOM | 482 | CD2 | LEU | A | 66 | 9.545 | 6.542 | −0.765 | 1.00 | 10.00 | A | C |
| ATOM | 483 | C | LEU | A | 66 | 7.784 | 8.531 | −4.314 | 1.00 | 10.00 | A | C |
| ATOM | 484 | O | LEU | A | 66 | 7.117 | 9.407 | −3.756 | 1.00 | 10.00 | A | O |
| ATOM | 485 | N | SER | A | 67 | 8.051 | 8.537 | −5.614 | 1.00 | 10.00 | A | N |
| ATOM | 487 | CA | SER | A | 67 | 7.580 | 9.597 | −6.489 | 1.00 | 10.00 | A | C |
| ATOM | 488 | CB | SER | A | 67 | 8.344 | 9.572 | −7.819 | 1.00 | 10.00 | A | C |
| ATOM | 489 | OG | SER | A | 67 | 9.741 | 9.492 | −7.603 | 1.00 | 10.00 | A | O |
| ATOM | 491 | C | SER | A | 67 | 6.080 | 9.451 | −6.738 | 1.00 | 10.00 | A | C |
| ATOM | 492 | O | SER | A | 67 | 5.349 | 10.441 | −6.762 | 1.00 | 10.00 | A | O |
| ATOM | 493 | N | THR | A | 68 | 5.634 | 8.208 | −6.909 | 1.00 | 10.00 | A | N |
| ATOM | 495 | CA | THR | A | 68 | 4.227 | 7.914 | −7.158 | 1.00 | 10.00 | A | C |
| ATOM | 496 | CB | THR | A | 68 | 4.016 | 6.414 | −7.453 | 1.00 | 10.00 | A | C |
| ATOM | 497 | OG1 | THR | A | 68 | 4.997 | 5.974 | −8.403 | 1.00 | 10.00 | A | O |
| ATOM | 499 | CG2 | THR | A | 68 | 2.626 | 6.161 | −8.020 | 1.00 | 10.00 | A | C |
| ATOM | 500 | C | THR | A | 68 | 3.344 | 8.339 | −5.980 | 1.00 | 10.00 | A | C |
| ATOM | 501 | O | THR | A | 68 | 2.201 | 8.770 | −6.170 | 1.00 | 10.00 | A | O |
| ATOM | 502 | N | LEU | A | 69 | 3.875 | 8.223 | −4.767 | 1.00 | 10.00 | A | N |
| ATOM | 504 | CA | LEU | A | 69 | 3.129 | 8.604 | −3.578 | 1.00 | 10.00 | A | C |
| ATOM | 505 | CB | LEU | A | 69 | 3.513 | 7.734 | −2.380 | 1.00 | 10.00 | A | C |
| ATOM | 506 | CG | LEU | A | 69 | 2.639 | 6.498 | −2.136 | 1.00 | 10.00 | A | C |
| ATOM | 507 | CD1 | LEU | A | 69 | 2.896 | 5.426 | −3.186 | 1.00 | 10.00 | A | C |
| ATOM | 508 | CD2 | LEU | A | 69 | 2.865 | 5.947 | −0.737 | 1.00 | 10.00 | A | C |
| ATOM | 509 | C | LEU | A | 69 | 3.309 | 10.086 | −3.264 | 1.00 | 10.00 | A | C |
| ATOM | 510 | O | LEU | A | 69 | 2.750 | 10.596 | −2.292 | 1.00 | 10.00 | A | O |
| ATOM | 511 | N | SER | A | 70 | 4.082 | 10.771 | −4.107 | 1.00 | 10.00 | A | N |
| ATOM | 513 | CA | SER | A | 70 | 4.342 | 12.197 | −3.954 | 1.00 | 10.00 | A | C |
| ATOM | 514 | CB | SER | A | 70 | 3.062 | 13.006 | −4.170 | 1.00 | 10.00 | A | C |
| ATOM | 515 | OG | SER | A | 70 | 2.358 | 12.557 | −5.319 | 1.00 | 10.00 | A | O |
| ATOM | 517 | C | SER | A | 70 | 4.972 | 12.532 | −2.604 | 1.00 | 10.00 | A | C |
| ATOM | 518 | O | SER | A | 70 | 4.453 | 13.354 | −1.846 | 1.00 | 10.00 | A | O |
| ATOM | 519 | N | MET | A | 71 | 6.087 | 11.886 | −2.303 | 1.00 | 10.00 | A | N |
| ATOM | 521 | CA | MET | A | 71 | 6.797 | 12.143 | −1.051 | 1.00 | 10.00 | A | C |
| ATOM | 522 | CB | MET | A | 71 | 7.595 | 10.915 | −0.601 | 1.00 | 10.00 | A | C |
| ATOM | 523 | CG | MET | A | 71 | 6.804 | 9.943 | 0.259 | 1.00 | 10.00 | A | C |
| ATOM | 524 | SD | MET | A | 71 | 6.243 | 10.673 | 1.813 | 1.00 | 10.00 | A | S |
| ATOM | 525 | CE | MET | A | 71 | 7.804 | 10.940 | 2.655 | 1.00 | 10.00 | A | C |
| ATOM | 526 | C | MET | A | 71 | 7.721 | 13.353 | −1.202 | 1.00 | 10.00 | A | C |
| ATOM | 527 | O | MET | A | 71 | 7.702 | 14.034 | −2.226 | 1.00 | 10.00 | A | O |
| ATOM | 528 | N | ASP | A | 72 | 8.522 | 13.628 | −0.181 | 1.00 | 10.00 | A | N |
| ATOM | 530 | CA | ASP | A | 72 | 9.447 | 14.750 | −0.228 | 1.00 | 10.00 | A | C |
| ATOM | 531 | CB | ASP | A | 72 | 9.819 | 15.198 | 1.187 | 1.00 | 10.00 | A | C |
| ATOM | 532 | CG | ASP | A | 72 | 10.444 | 16.583 | 1.238 | 1.00 | 10.00 | A | C |
| ATOM | 533 | OD1 | ASP | A | 72 | 11.503 | 16.796 | 0.609 | 1.00 | 10.00 | A | O |
| ATOM | 534 | OD2 | ASP | A | 72 | 9.879 | 17.467 | 1.923 | 1.00 | 10.00 | A | O |
| ATOM | 535 | C | ASP | A | 72 | 10.696 | 14.342 | −0.997 | 1.00 | 10.00 | A | C |
| ATOM | 536 | O | ASP | A | 72 | 11.081 | 13.169 | −0.990 | 1.00 | 10.00 | A | O |
| ATOM | 537 | N | ILE | A | 73 | 11.328 | 15.303 | −1.653 | 1.00 | 10.00 | A | N |
| ATOM | 539 | CA | ILE | A | 73 | 12.527 | 15.039 | −2.435 | 1.00 | 10.00 | A | C |
| ATOM | 540 | CB | ILE | A | 73 | 13.010 | 16.271 | −3.227 | 1.00 | 10.00 | A | C |
| ATOM | 541 | CG1 | ILE | A | 73 | 13.095 | 17.508 | −2.326 | 1.00 | 10.00 | A | C |
| ATOM | 542 | CG2 | ILE | A | 73 | 12.088 | 16.525 | −4.410 | 1.00 | 10.00 | A | C |
| ATOM | 543 | CD1 | ILE | A | 73 | 14.498 | 17.841 | −1.862 | 1.00 | 10.00 | A | C |
| ATOM | 544 | C | ILE | A | 73 | 13.655 | 14.511 | −1.560 | 1.00 | 10.00 | A | C |
| ATOM | 545 | O | ILE | A | 73 | 14.529 | 13.792 | −2.037 | 1.00 | 10.00 | A | O |
| ATOM | 546 | N | ASP | A | 74 | 13.626 | 14.856 | −0.278 | 1.00 | 10.00 | A | N |
| ATOM | 548 | CA | ASP | A | 74 | 14.652 | 14.398 | 0.654 | 1.00 | 10.00 | A | C |
| ATOM | 549 | CB | ASP | A | 74 | 14.633 | 15.171 | 1.979 | 1.00 | 10.00 | A | C |
| ATOM | 550 | CG | ASP | A | 74 | 13.345 | 15.047 | 2.783 | 1.00 | 10.00 | A | C |
| ATOM | 551 | OD1 | ASP | A | 74 | 12.566 | 14.101 | 2.554 | 1.00 | 10.00 | A | O |
| ATOM | 552 | OD2 | ASP | A | 74 | 13.108 | 15.914 | 3.654 | 1.00 | 10.00 | A | O |
| ATOM | 553 | C | ASP | A | 74 | 14.540 | 12.897 | 0.885 | 1.00 | 10.00 | A | C |
| ATOM | 554 | O | ASP | A | 74 | 15.518 | 12.229 | 1.220 | 1.00 | 10.00 | A | O |
| ATOM | 555 | N | THR | A | 75 | 13.343 | 12.371 | 0.686 | 1.00 | 10.00 | A | N |
| ATOM | 557 | CA | THR | A | 75 | 13.097 | 10.957 | 0.854 | 1.00 | 10.00 | A | C |
| ATOM | 558 | CB | THR | A | 75 | 11.608 | 10.701 | 1.129 | 1.00 | 10.00 | A | C |
| ATOM | 559 | OG1 | THR | A | 75 | 11.064 | 11.805 | 1.871 | 1.00 | 10.00 | A | O |
| ATOM | 561 | CG2 | THR | A | 75 | 11.433 | 9.425 | 1.936 | 1.00 | 10.00 | A | C |
| ATOM | 562 | C | THR | A | 75 | 13.515 | 10.224 | −0.416 | 1.00 | 10.00 | A | C |
| ATOM | 563 | O | THR | A | 75 | 14.048 | 9.116 | −0.366 | 1.00 | 10.00 | A | O |
| ATOM | 564 | N | LEU | A | 76 | 13.286 | 10.875 | −1.554 | 1.00 | 10.00 | A | N |
| ATOM | 566 | CA | LEU | A | 76 | 13.642 | 10.316 | −2.854 | 1.00 | 10.00 | A | C |
| ATOM | 567 | CB | LEU | A | 76 | 13.153 | 11.227 | −3.989 | 1.00 | 10.00 | A | C |
| ATOM | 568 | CG | LEU | A | 76 | 11.781 | 10.899 | −4.584 | 1.00 | 10.00 | A | C |
| ATOM | 569 | CD1 | LEU | A | 76 | 10.667 | 11.250 | −3.614 | 1.00 | 10.00 | A | C |
| ATOM | 570 | CD2 | LEU | A | 76 | 11.587 | 11.609 | −5.912 | 1.00 | 10.00 | A | C |
| ATOM | 571 | C | LEU | A | 76 | 15.153 | 10.140 | −2.954 | 1.00 | 10.00 | A | C |
| ATOM | 572 | O | LEU | A | 76 | 15.642 | 9.131 | −3.466 | 1.00 | 10.00 | A | O |
| ATOM | 573 | N | ARG | A | 77 | 15.882 | 11.125 | −2.439 | 1.00 | 10.00 | A | N |
| ATOM | 575 | CA | ARG | A | 77 | 17.336 | 11.097 | −2.452 | 1.00 | 10.00 | A | C |
| ATOM | 576 | CB | ARG | A | 77 | 17.901 | 12.386 | −1.848 | 1.00 | 10.00 | A | C |
| ATOM | 577 | CG | ARG | A | 77 | 17.540 | 13.646 | −2.610 | 1.00 | 10.00 | A | C |
| ATOM | 578 | CD | ARG | A | 77 | 18.107 | 14.880 | −1.931 | 1.00 | 10.00 | A | C |
| ATOM | 579 | NE | ARG | A | 77 | 19.526 | 15.054 | −2.222 | 1.00 | 10.00 | A | N |
| ATOM | 581 | CZ | ARG | A | 77 | 20.247 | 16.090 | −1.810 | 1.00 | 10.00 | A | C |
| ATOM | 582 | NH1 | ARG | A | 77 | 19.691 | 17.055 | −1.088 | 1.00 | 10.00 | A | N |
| ATOM | 585 | NH2 | ARG | A | 77 | 21.525 | 16.172 | −2.137 | 1.00 | 10.00 | A | N |
| ATOM | 588 | C | ARG | A | 77 | 17.857 | 9.910 | −1.660 | 1.00 | 10.00 | A | C |
| ATOM | 589 | O | ARG | A | 77 | 18.817 | 9.263 | −2.061 | 1.00 | 10.00 | A | O |
| ATOM | 590 | N | ALA | A | 78 | 17.201 | 9.624 | −0.542 | 1.00 | 10.00 | A | N |
| ATOM | 592 | CA | ALA | A | 78 | 17.596 | 8.521 | 0.327 | 1.00 | 10.00 | A | C |
| ATOM | 593 | CB | ALA | A | 78 | 16.727 | 8.490 | 1.572 | 1.00 | 10.00 | A | C |
| ATOM | 594 | C | ALA | A | 78 | 17.543 | 7.179 | −0.391 | 1.00 | 10.00 | A | C |
| ATOM | 595 | O | ALA | A | 78 | 18.413 | 6.336 | −0.201 | 1.00 | 10.00 | A | O |
| ATOM | 596 | N | ALA | A | 79 | 16.521 | 6.992 | −1.221 | 1.00 | 10.00 | A | N |
| ATOM | 598 | CA | ALA | A | 79 | 16.367 | 5.744 | −1.961 | 1.00 | 10.00 | A | C |
| ATOM | 599 | CB | ALA | A | 79 | 14.976 | 5.647 | −2.560 | 1.00 | 10.00 | A | C |
| ATOM | 600 | C | ALA | A | 79 | 17.436 | 5.610 | −3.041 | 1.00 | 10.00 | A | C |
| ATOM | 601 | O | ALA | A | 79 | 17.885 | 4.512 | −3.355 | 1.00 | 10.00 | A | O |
| ATOM | 602 | N | LEU | A | 80 | 17.851 | 6.735 | −3.600 | 1.00 | 10.00 | A | N |
| ATOM | 604 | CA | LEU | A | 80 | 18.875 | 6.737 | −4.637 | 1.00 | 10.00 | A | C |
| ATOM | 605 | CB | LEU | A | 80 | 18.848 | 8.056 | −5.410 | 1.00 | 10.00 | A | C |
| ATOM | 606 | CG | LEU | A | 80 | 17.827 | 8.147 | −6.546 | 1.00 | 10.00 | A | C |
| ATOM | 607 | CD1 | LEU | A | 80 | 17.532 | 9.595 | −6.909 | 1.00 | 10.00 | A | C |
| ATOM | 608 | CD2 | LEU | A | 80 | 18.299 | 7.365 | −7.758 | 1.00 | 10.00 | A | C |
| ATOM | 609 | C | LEU | A | 80 | 20.252 | 6.517 | −4.028 | 1.00 | 10.00 | A | C |
| ATOM | 610 | O | LEU | A | 80 | 21.107 | 5.848 | −4.605 | 1.00 | 10.00 | A | O |
| ATOM | 611 | N | LEU | A | 81 | 20.454 | 7.071 | −2.846 | 1.00 | 10.00 | A | N |
| ATOM | 613 | CA | LEU | A | 81 | 21.725 | 6.948 | −2.151 | 1.00 | 10.00 | A | C |
| ATOM | 614 | CB | LEU | A | 81 | 21.998 | 8.201 | −1.322 | 1.00 | 10.00 | A | C |
| ATOM | 615 | CG | LEU | A | 81 | 21.849 | 9.544 | −2.044 | 1.00 | 10.00 | A | C |
| ATOM | 616 | CD1 | LEU | A | 81 | 21.946 | 10.691 | −1.052 | 1.00 | 10.00 | A | C |
| ATOM | 617 | CD2 | LEU | A | 81 | 22.897 | 9.693 | −3.136 | 1.00 | 10.00 | A | C |
| ATOM | 618 | C | LEU | A | 81 | 21.739 | 5.721 | −1.248 | 1.00 | 10.00 | A | C |
| ATOM | 619 | O | LEU | A | 81 | 22.691 | 5.510 | −0.495 | 1.00 | 10.00 | A | O |
| ATOM | 620 | N | PHE | A | 82 | 20.681 | 4.921 | −1.326 | 1.00 | 10.00 | A | N |
| ATOM | 622 | CA | PHE | A | 82 | 20.565 | 3.715 | −0.514 | 1.00 | 10.00 | A | C |
| ATOM | 623 | CB | PHE | A | 82 | 19.160 | 3.098 | −0.598 | 1.00 | 10.00 | A | C |
| ATOM | 624 | CG | PHE | A | 82 | 19.031 | 1.771 | 0.096 | 1.00 | 10.00 | A | C |
| ATOM | 625 | CD1 | PHE | A | 82 | 19.325 | 1.647 | 1.442 | 1.00 | 10.00 | A | C |
| ATOM | 626 | CD2 | PHE | A | 82 | 18.622 | 0.646 | −0.599 | 1.00 | 10.00 | A | C |
| ATOM | 627 | CE1 | PHE | A | 82 | 19.213 | 0.426 | 2.084 | 1.00 | 10.00 | A | C |
| ATOM | 628 | CE2 | PHE | A | 82 | 18.509 | −0.579 | 0.035 | 1.00 | 10.00 | A | C |
| ATOM | 629 | CZ | PHE | A | 82 | 18.804 | −0.689 | 1.379 | 1.00 | 10.00 | A | C |
| ATOM | 630 | C | PHE | A | 82 | 21.667 | 2.692 | −0.824 | 1.00 | 10.00 | A | C |
| ATOM | 631 | O | PHE | A | 82 | 22.438 | 2.332 | 0.066 | 1.00 | 10.00 | A | O |
| ATOM | 632 | N | PRO | A | 83 | 21.785 | 2.217 | −2.083 | 1.00 | 10.00 | A | N |
| ATOM | 633 | CA | PRO | A | 83 | 22.808 | 1.242 | −2.460 | 1.00 | 10.00 | A | C |
| ATOM | 634 | CB | PRO | A | 83 | 22.273 | 0.639 | −3.776 | 1.00 | 10.00 | A | C |
| ATOM | 635 | CG | PRO | A | 83 | 20.957 | 1.310 | −4.044 | 1.00 | 10.00 | A | C |
| ATOM | 636 | CD | PRO | A | 83 | 20.954 | 2.577 | −3.242 | 1.00 | 10.00 | A | C |
| ATOM | 637 | C | PRO | A | 83 | 24.162 | 1.921 | −2.699 | 1.00 | 10.00 | A | C |
| ATOM | 638 | O | PRO | A | 83 | 24.725 | 1.835 | −3.789 | 1.00 | 10.00 | A | O |
| ATOM | 639 | N | LEU | A | 84 | 24.667 | 2.615 | −1.684 | 1.00 | 10.00 | A | N |
| ATOM | 641 | CA | LEU | A | 84 | 25.952 | 3.301 | −1.798 | 1.00 | 10.00 | A | C |
| ATOM | 642 | CB | LEU | A | 84 | 25.753 | 4.810 | −1.960 | 1.00 | 10.00 | A | C |
| ATOM | 643 | CG | LEU | A | 84 | 25.243 | 5.283 | −3.326 | 1.00 | 10.00 | A | C |
| ATOM | 644 | CD1 | LEU | A | 84 | 25.087 | 6.796 | −3.346 | 1.00 | 10.00 | A | C |
| ATOM | 645 | CD2 | LEU | A | 84 | 26.179 | 4.827 | −4.436 | 1.00 | 10.00 | A | C |
| ATOM | 646 | C | LEU | A | 84 | 26.853 | 3.017 | −0.600 | 1.00 | 10.00 | A | C |
| ATOM | 647 | O | LEU | A | 84 | 27.993 | 2.573 | −0.760 | 1.00 | 10.00 | A | O |
| ATOM | 648 | N | ALA | A | 85 | 26.324 | 3.255 | 0.595 | 1.00 | 10.00 | A | N |
| ATOM | 650 | CA | ALA | A | 85 | 27.072 | 3.051 | 1.838 | 1.00 | 10.00 | A | C |
| ATOM | 651 | CB | ALA | A | 85 | 26.194 | 3.345 | 3.045 | 1.00 | 10.00 | A | C |
| ATOM | 652 | C | ALA | A | 85 | 27.684 | 1.654 | 1.941 | 1.00 | 10.00 | A | C |
| ATOM | 653 | O | ALA | A | 85 | 28.833 | 1.499 | 2.361 | 1.00 | 10.00 | A | O |
| ATOM | 654 | N | ASP | A | 86 | 26.924 | 0.645 | 1.545 | 1.00 | 10.00 | A | N |
| ATOM | 656 | CA | ASP | A | 86 | 27.388 | −0.737 | 1.601 | 1.00 | 10.00 | A | C |
| ATOM | 657 | CB | ASP | A | 86 | 26.412 | −1.604 | 2.403 | 1.00 | 10.00 | A | C |
| ATOM | 658 | CG | ASP | A | 86 | 26.453 | −1.326 | 3.891 | 1.00 | 10.00 | A | C |
| ATOM | 659 | OD1 | ASP | A | 86 | 25.377 | −1.134 | 4.492 | 1.00 | 10.00 | A | O |
| ATOM | 660 | OD2 | ASP | A | 86 | 27.560 | −1.293 | 4.466 | 1.00 | 10.00 | A | O |
| ATOM | 661 | C | ASP | A | 86 | 27.563 | −1.310 | 0.204 | 1.00 | 10.00 | A | C |
| ATOM | 662 | O | ASP | A | 86 | 27.372 | −2.510 | −0.021 | 1.00 | 10.00 | A | O |
| ATOM | 663 | N | ALA | A | 87 | 27.926 | −0.450 | −0.734 | 1.00 | 10.00 | A | N |
| ATOM | 665 | CA | ALA | A | 87 | 28.130 | −0.866 | −2.114 | 1.00 | 10.00 | A | C |
| ATOM | 666 | CB | ALA | A | 87 | 26.933 | −0.488 | −2.971 | 1.00 | 10.00 | A | C |
| ATOM | 667 | C | ALA | A | 87 | 29.404 | −0.263 | −2.688 | 1.00 | 10.00 | A | C |
| ATOM | 668 | O | ALA | A | 87 | 30.159 | −0.938 | −3.389 | 1.00 | 10.00 | A | O |
| ATOM | 669 | N | ASN | A | 88 | 29.649 | 1.001 | −2.384 | 1.00 | 10.00 | A | N |
| ATOM | 671 | CA | ASN | A | 88 | 30.835 | 1.681 | −2.881 | 1.00 | 10.00 | A | C |
| ATOM | 672 | CB | ASN | A | 88 | 30.455 | 2.913 | −3.700 | 1.00 | 10.00 | A | C |
| ATOM | 673 | CG | ASN | A | 88 | 29.849 | 2.565 | −5.042 | 1.00 | 10.00 | A | C |
| ATOM | 674 | OD1 | ASN | A | 88 | 28.628 | 2.521 | −5.193 | 1.00 | 10.00 | A | O |
| ATOM | 675 | ND2 | ASN | A | 88 | 30.695 | 2.315 | −6.030 | 1.00 | 10.00 | A | N |
| ATOM | 678 | C | ASN | A | 88 | 31.755 | 2.074 | −1.739 | 1.00 | 10.00 | A | C |
| ATOM | 679 | O | ASN | A | 88 | 31.528 | 1.694 | −0.588 | 1.00 | 10.00 | A | O |
| ATOM | 680 | N | VAL | A | 89 | 32.801 | 2.823 | −2.065 | 1.00 | 10.00 | A | N |
| ATOM | 682 | CA | VAL | A | 89 | 33.763 | 3.273 | −1.070 | 1.00 | 10.00 | A | C |
| ATOM | 683 | CB | VAL | A | 89 | 35.135 | 3.605 | −1.715 | 1.00 | 10.00 | A | C |
| ATOM | 684 | CG1 | VAL | A | 89 | 36.202 | 3.832 | −0.654 | 1.00 | 10.00 | A | C |
| ATOM | 685 | CG2 | VAL | A | 89 | 35.565 | 2.504 | −2.673 | 1.00 | 10.00 | A | C |
| ATOM | 686 | C | VAL | A | 89 | 33.225 | 4.508 | −0.356 | 1.00 | 10.00 | A | C |
| ATOM | 687 | O | VAL | A | 89 | 33.660 | 4.847 | 0.743 | 1.00 | 10.00 | A | O |
| ATOM | 688 | N | VAL | A | 90 | 32.271 | 5.173 | −0.991 | 1.00 | 10.00 | A | N |
| ATOM | 690 | CA | VAL | A | 90 | 31.664 | 6.370 | −0.426 | 1.00 | 10.00 | A | C |
| ATOM | 691 | CB | VAL | A | 90 | 30.739 | 7.075 | −1.446 | 1.00 | 10.00 | A | C |
| ATOM | 692 | CG1 | VAL | A | 90 | 29.600 | 6.164 | −1.881 | 1.00 | 10.00 | A | C |
| ATOM | 693 | CG2 | VAL | A | 90 | 30.207 | 8.392 | −0.897 | 1.00 | 10.00 | A | C |
| ATOM | 694 | C | VAL | A | 90 | 30.893 | 6.037 | 0.854 | 1.00 | 10.00 | A | C |
| ATOM | 695 | O | VAL | A | 90 | 30.101 | 5.093 | 0.896 | 1.00 | 10.00 | A | O |
| ATOM | 696 | N | SER | A | 91 | 31.153 | 6.799 | 1.899 | 1.00 | 10.00 | A | N |
| ATOM | 698 | CA | SER | A | 91 | 30.495 | 6.590 | 3.172 | 1.00 | 10.00 | A | C |
| ATOM | 699 | CB | SER | A | 91 | 31.547 | 6.543 | 4.281 | 1.00 | 10.00 | A | C |
| ATOM | 700 | OG | SER | A | 91 | 32.294 | 7.750 | 4.319 | 1.00 | 10.00 | A | O |
| ATOM | 702 | C | SER | A | 91 | 29.503 | 7.711 | 3.447 | 1.00 | 10.00 | A | C |
| ATOM | 703 | O | SER | A | 91 | 29.447 | 8.702 | 2.710 | 1.00 | 10.00 | A | O |
| ATOM | 704 | N | GLU | A | 92 | 28.727 | 7.559 | 4.511 | 1.00 | 10.00 | A | N |
| ATOM | 706 | CA | GLU | A | 92 | 27.749 | 8.566 | 4.896 | 1.00 | 10.00 | A | C |
| ATOM | 707 | CB | GLU | A | 92 | 26.839 | 8.059 | 6.017 | 1.00 | 10.00 | A | C |
| ATOM | 708 | CG | GLU | A | 92 | 27.564 | 7.324 | 7.135 | 1.00 | 10.00 | A | C |
| ATOM | 709 | CD | GLU | A | 92 | 27.735 | 5.847 | 6.844 | 1.00 | 10.00 | A | C |
| ATOM | 710 | OE1 | GLU | A | 92 | 28.752 | 5.471 | 6.218 | 1.00 | 10.00 | A | O |
| ATOM | 711 | OE2 | GLU | A | 92 | 26.856 | 5.057 | 7.238 | 1.00 | 10.00 | A | O |
| ATOM | 712 | C | GLU | A | 92 | 28.451 | 9.854 | 5.316 | 1.00 | 10.00 | A | C |
| ATOM | 713 | O | GLU | A | 92 | 27.854 | 10.929 | 5.316 | 1.00 | 10.00 | A | O |
| ATOM | 714 | N | ASP | A | 93 | 29.734 | 9.730 | 5.651 | 1.00 | 10.00 | A | N |
| ATOM | 716 | CA | ASP | A | 93 | 30.537 | 10.872 | 6.054 | 1.00 | 10.00 | A | C |
| ATOM | 717 | CB | ASP | A | 93 | 31.882 | 10.408 | 6.611 | 1.00 | 10.00 | A | C |
| ATOM | 718 | CG | ASP | A | 93 | 32.300 | 11.214 | 7.816 | 1.00 | 10.00 | A | C |
| ATOM | 719 | OD1 | ASP | A | 93 | 32.841 | 12.326 | 7.640 | 1.00 | 10.00 | A | O |
| ATOM | 720 | OD2 | ASP | A | 93 | 32.079 | 10.751 | 8.951 | 1.00 | 10.00 | A | O |
| ATOM | 721 | C | ASP | A | 93 | 30.755 | 11.779 | 4.853 | 1.00 | 10.00 | A | C |
| ATOM | 722 | O | ASP | A | 93 | 30.722 | 13.004 | 4.964 | 1.00 | 10.00 | A | O |
| ATOM | 723 | N | VAL | A | 94 | 30.973 | 11.154 | 3.701 | 1.00 | 10.00 | A | N |
| ATOM | 725 | CA | VAL | A | 94 | 31.164 | 11.881 | 2.454 | 1.00 | 10.00 | A | C |
| ATOM | 726 | CB | VAL | A | 94 | 31.849 | 11.009 | 1.384 | 1.00 | 10.00 | A | C |
| ATOM | 727 | CG1 | VAL | A | 94 | 32.025 | 11.776 | 0.083 | 1.00 | 10.00 | A | C |
| ATOM | 728 | CG2 | VAL | A | 94 | 33.189 | 10.514 | 1.897 | 1.00 | 10.00 | A | C |
| ATOM | 729 | C | VAL | A | 94 | 29.814 | 12.354 | 1.943 | 1.00 | 10.00 | A | C |
| ATOM | 730 | O | VAL | A | 94 | 29.684 | 13.451 | 1.402 | 1.00 | 10.00 | A | O |
| ATOM | 731 | N | LEU | A | 95 | 28.799 | 11.522 | 2.138 | 1.00 | 10.00 | A | N |
| ATOM | 733 | CA | LEU | A | 95 | 27.448 | 11.861 | 1.723 | 1.00 | 10.00 | A | C |
| ATOM | 734 | CB | LEU | A | 95 | 26.501 | 10.684 | 1.960 | 1.00 | 10.00 | A | C |
| ATOM | 735 | CG | LEU | A | 95 | 26.701 | 9.468 | 1.049 | 1.00 | 10.00 | A | C |
| ATOM | 736 | CD1 | LEU | A | 95 | 25.859 | 8.294 | 1.526 | 1.00 | 10.00 | A | C |
| ATOM | 737 | CD2 | LEU | A | 95 | 26.364 | 9.819 | −0.390 | 1.00 | 10.00 | A | C |
| ATOM | 738 | C | LEU | A | 95 | 26.968 | 13.093 | 2.479 | 1.00 | 10.00 | A | C |
| ATOM | 739 | O | LEU | A | 95 | 26.164 | 13.866 | 1.971 | 1.00 | 10.00 | A | O |
| ATOM | 740 | N | ARG | A | 96 | 27.503 | 13.283 | 3.682 | 1.00 | 10.00 | A | N |
| ATOM | 742 | CA | ARG | A | 96 | 27.155 | 14.427 | 4.525 | 1.00 | 10.00 | A | C |
| ATOM | 743 | CB | ARG | A | 96 | 27.802 | 14.274 | 5.904 | 1.00 | 10.00 | A | C |
| ATOM | 744 | CG | ARG | A | 96 | 27.312 | 15.266 | 6.948 | 1.00 | 10.00 | A | C |
| ATOM | 745 | CD | ARG | A | 96 | 28.340 | 15.460 | 8.049 | 1.00 | 10.00 | A | C |
| ATOM | 746 | NE | ARG | A | 96 | 28.590 | 14.228 | 8.798 | 1.00 | 10.00 | A | N |
| ATOM | 748 | CZ | ARG | A | 96 | 29.731 | 13.547 | 8.750 | 1.00 | 10.00 | A | C |
| ATOM | 749 | NH1 | ARG | A | 96 | 30.727 | 13.978 | 7.988 | 1.00 | 10.00 | A | N |
| ATOM | 752 | NH2 | ARG | A | 96 | 29.876 | 12.438 | 9.464 | 1.00 | 10.00 | A | N |
| ATOM | 755 | C | ARG | A | 96 | 27.586 | 15.749 | 3.878 | 1.00 | 10.00 | A | C |
| ATOM | 756 | O | ARG | A | 96 | 27.183 | 16.827 | 4.308 | 1.00 | 10.00 | A | O |
| ATOM | 757 | N | GLU | A | 97 | 28.424 | 15.657 | 2.856 | 1.00 | 10.00 | A | N |
| ATOM | 759 | CA | GLU | A | 97 | 28.891 | 16.834 | 2.139 | 1.00 | 10.00 | A | C |
| ATOM | 760 | CB | GLU | A | 97 | 30.242 | 16.555 | 1.471 | 1.00 | 10.00 | A | C |
| ATOM | 761 | CG | GLU | A | 97 | 31.368 | 16.216 | 2.439 | 1.00 | 10.00 | A | C |
| ATOM | 762 | CD | GLU | A | 97 | 32.676 | 15.883 | 1.742 | 1.00 | 10.00 | A | C |
| ATOM | 763 | OE1 | GLU | A | 97 | 32.775 | 16.098 | 0.514 | 1.00 | 10.00 | A | O |
| ATOM | 764 | OE2 | GLU | A | 97 | 33.622 | 15.419 | 2.418 | 1.00 | 10.00 | A | O |
| ATOM | 765 | C | GLU | A | 97 | 27.868 | 17.230 | 1.080 | 1.00 | 10.00 | A | C |
| ATOM | 766 | O | GLU | A | 97 | 27.591 | 18.410 | 0.869 | 1.00 | 10.00 | A | O |
| ATOM | 767 | N | SER | A | 98 | 27.294 | 16.229 | 0.430 | 1.00 | 10.00 | A | N |
| ATOM | 769 | CA | SER | A | 98 | 26.315 | 16.461 | −0.619 | 1.00 | 10.00 | A | C |
| ATOM | 770 | CB | SER | A | 98 | 26.351 | 15.298 | −1.612 | 1.00 | 10.00 | A | C |
| ATOM | 771 | OG | SER | A | 98 | 27.628 | 14.683 | −1.621 | 1.00 | 10.00 | A | O |
| ATOM | 773 | C | SER | A | 98 | 24.902 | 16.621 | −0.053 | 1.00 | 10.00 | A | C |
| ATOM | 774 | O | SER | A | 98 | 24.119 | 17.444 | −0.530 | 1.00 | 10.00 | A | O |
| ATOM | 775 | N | VAL | A | 99 | 24.579 | 15.839 | 0.968 | 1.00 | 10.00 | A | N |
| ATOM | 777 | CA | VAL | A | 99 | 23.253 | 15.892 | 1.575 | 1.00 | 10.00 | A | C |
| ATOM | 778 | CB | VAL | A | 99 | 22.524 | 14.530 | 1.503 | 1.00 | 10.00 | A | C |
| ATOM | 779 | CG1 | VAL | A | 99 | 22.517 | 14.003 | 0.083 | 1.00 | 10.00 | A | C |
| ATOM | 780 | CG2 | VAL | A | 99 | 23.144 | 13.510 | 2.449 | 1.00 | 10.00 | A | C |
| ATOM | 781 | C | VAL | A | 99 | 23.338 | 16.345 | 3.024 | 1.00 | 10.00 | A | C |
| ATOM | 782 | O | VAL | A | 99 | 24.390 | 16.244 | 3.650 | 1.00 | 10.00 | A | O |
| ATOM | 783 | N | GLY | A | 100 | 22.242 | 16.870 | 3.545 | 1.00 | 10.00 | A | N |
| ATOM | 785 | CA | GLY | A | 100 | 22.220 | 17.317 | 4.924 | 1.00 | 10.00 | A | C |
| ATOM | 786 | C | GLY | A | 100 | 22.070 | 16.150 | 5.881 | 1.00 | 10.00 | A | C |
| ATOM | 787 | O | GLY | A | 100 | 21.809 | 15.021 | 5.454 | 1.00 | 10.00 | A | O |
| ATOM | 788 | N | LYS | A | 101 | 22.222 | 16.418 | 7.174 | 1.00 | 10.00 | A | N |
| ATOM | 790 | CA | LYS | A | 101 | 22.110 | 15.379 | 8.195 | 1.00 | 10.00 | A | C |
| ATOM | 791 | CB | LYS | A | 101 | 22.341 | 15.955 | 9.597 | 1.00 | 10.00 | A | C |
| ATOM | 792 | CG | LYS | A | 101 | 23.769 | 16.409 | 9.862 | 1.00 | 10.00 | A | C |
| ATOM | 793 | CD | LYS | A | 101 | 23.937 | 16.895 | 11.296 | 1.00 | 10.00 | A | C |
| ATOM | 794 | CE | LYS | A | 101 | 25.366 | 17.336 | 11.573 | 1.00 | 10.00 | A | C |
| ATOM | 795 | NZ | LYS | A | 101 | 25.548 | 17.821 | 12.969 | 1.00 | 10.00 | A | N |
| ATOM | 799 | C | LYS | A | 101 | 20.758 | 14.673 | 8.132 | 1.00 | 10.00 | A | C |
| ATOM | 800 | O | LYS | A | 101 | 20.641 | 13.509 | 8.517 | 1.00 | 10.00 | A | O |
| ATOM | 801 | N | SER | A | 102 | 19.748 | 15.386 | 7.636 | 1.00 | 10.00 | A | N |
| ATOM | 803 | CA | SER | A | 102 | 18.399 | 14.842 | 7.503 | 1.00 | 10.00 | A | C |
| ATOM | 804 | CB | SER | A | 102 | 17.446 | 15.941 | 7.034 | 1.00 | 10.00 | A | C |
| ATOM | 805 | OG | SER | A | 102 | 17.767 | 17.177 | 7.657 | 1.00 | 10.00 | A | O |
| ATOM | 807 | C | SER | A | 102 | 18.366 | 13.663 | 6.529 | 1.00 | 10.00 | A | C |
| ATOM | 808 | O | SER | A | 102 | 17.553 | 12.752 | 6.665 | 1.00 | 10.00 | A | O |
| ATOM | 809 | N | VAL | A | 103 | 19.261 | 13.680 | 5.550 | 1.00 | 10.00 | A | N |
| ATOM | 811 | CA | VAL | A | 103 | 19.329 | 12.603 | 4.569 | 1.00 | 10.00 | A | C |
| ATOM | 812 | CB | VAL | A | 103 | 19.756 | 13.113 | 3.172 | 1.00 | 10.00 | A | C |
| ATOM | 813 | CG1 | VAL | A | 103 | 19.437 | 12.075 | 2.102 | 1.00 | 10.00 | A | C |
| ATOM | 814 | CG2 | VAL | A | 103 | 19.084 | 14.440 | 2.851 | 1.00 | 10.00 | A | C |
| ATOM | 815 | C | VAL | A | 103 | 20.306 | 11.540 | 5.051 | 1.00 | 10.00 | A | C |
| ATOM | 816 | O | VAL | A | 103 | 20.139 | 10.351 | 4.777 | 1.00 | 10.00 | A | O |
| ATOM | 817 | N | VAL | A | 104 | 21.315 | 11.981 | 5.795 | 1.00 | 10.00 | A | N |
| ATOM | 819 | CA | VAL | A | 104 | 22.326 | 11.079 | 6.337 | 1.00 | 10.00 | A | C |
| ATOM | 820 | CB | VAL | A | 104 | 23.460 | 11.847 | 7.046 | 1.00 | 10.00 | A | C |
| ATOM | 821 | CG1 | VAL | A | 104 | 24.465 | 10.888 | 7.659 | 1.00 | 10.00 | A | C |
| ATOM | 822 | CG2 | VAL | A | 104 | 24.154 | 12.789 | 6.075 | 1.00 | 10.00 | A | C |
| ATOM | 823 | C | VAL | A | 104 | 21.695 | 10.083 | 7.306 | 1.00 | 10.00 | A | C |
| ATOM | 824 | O | VAL | A | 104 | 21.969 | 8.884 | 7.242 | 1.00 | 10.00 | A | O |
| ATOM | 825 | N | ASN | A | 105 | 20.825 | 10.578 | 8.185 | 1.00 | 10.00 | A | N |
| ATOM | 827 | CA | ASN | A | 105 | 20.154 | 9.712 | 9.152 | 1.00 | 10.00 | A | C |
| ATOM | 828 | CB | ASN | A | 105 | 19.468 | 10.498 | 10.278 | 1.00 | 10.00 | A | C |
| ATOM | 829 | CG | ASN | A | 105 | 18.140 | 11.118 | 9.887 | 1.00 | 10.00 | A | C |
| ATOM | 830 | OD1 | ASN | A | 105 | 18.081 | 12.288 | 9.529 | 1.00 | 10.00 | A | O |
| ATOM | 831 | ND2 | ASN | A | 105 | 17.063 | 10.346 | 9.962 | 1.00 | 10.00 | A | N |
| ATOM | 834 | C | ASN | A | 105 | 19.185 | 8.750 | 8.473 | 1.00 | 10.00 | A | C |
| ATOM | 835 | O | ASN | A | 105 | 18.835 | 7.715 | 9.036 | 1.00 | 10.00 | A | O |
| ATOM | 836 | N | LEU | A | 106 | 18.754 | 9.087 | 7.265 | 1.00 | 10.00 | A | N |
| ATOM | 838 | CA | LEU | A | 106 | 17.847 | 8.222 | 6.527 | 1.00 | 10.00 | A | C |
| ATOM | 839 | CB | LEU | A | 106 | 17.265 | 8.938 | 5.305 | 1.00 | 10.00 | A | C |
| ATOM | 840 | CG | LEU | A | 106 | 16.386 | 10.162 | 5.566 | 1.00 | 10.00 | A | C |
| ATOM | 841 | CD1 | LEU | A | 106 | 15.515 | 10.467 | 4.358 | 1.00 | 10.00 | A | C |
| ATOM | 842 | CD2 | LEU | A | 106 | 15.534 | 9.974 | 6.813 | 1.00 | 10.00 | A | C |
| ATOM | 843 | C | LEU | A | 106 | 18.592 | 6.971 | 6.080 | 1.00 | 10.00 | A | C |
| ATOM | 844 | O | LEU | A | 106 | 18.140 | 5.850 | 6.310 | 1.00 | 10.00 | A | O |
| ATOM | 845 | N | ILE | A | 107 | 19.747 | 7.186 | 5.454 | 1.00 | 10.00 | A | N |
| ATOM | 847 | CA | ILE | A | 107 | 20.588 | 6.095 | 4.972 | 1.00 | 10.00 | A | C |
| ATOM | 848 | CB | ILE | A | 107 | 21.792 | 6.632 | 4.165 | 1.00 | 10.00 | A | C |
| ATOM | 849 | CG1 | ILE | A | 107 | 21.309 | 7.584 | 3.068 | 1.00 | 10.00 | A | C |
| ATOM | 850 | CG2 | ILE | A | 107 | 22.593 | 5.488 | 3.564 | 1.00 | 10.00 | A | C |
| ATOM | 851 | CD1 | ILE | A | 107 | 22.387 | 8.507 | 2.543 | 1.00 | 10.00 | A | C |
| ATOM | 852 | C | ILE | A | 107 | 21.090 | 5.272 | 6.157 | 1.00 | 10.00 | A | C |
| ATOM | 853 | O | ILE | A | 107 | 21.224 | 4.050 | 6.076 | 1.00 | 10.00 | A | O |
| ATOM | 854 | N | HIS | A | 108 | 21.347 | 5.961 | 7.261 | 1.00 | 10.00 | A | N |
| ATOM | 856 | CA | HIS | A | 108 | 21.812 | 5.319 | 8.482 | 1.00 | 10.00 | A | C |
| ATOM | 857 | CB | HIS | A | 108 | 22.288 | 6.378 | 9.485 | 1.00 | 10.00 | A | C |
| ATOM | 858 | CG | HIS | A | 108 | 22.872 | 5.826 | 10.748 | 1.00 | 10.00 | A | C |
| ATOM | 859 | ND1 | HIS | A | 108 | 22.302 | 6.084 | 11.973 | 1.00 | 10.00 | A | N |
| ATOM | 860 | CD2 | HIS | A | 108 | 23.974 | 5.056 | 10.925 | 1.00 | 10.00 | A | C |
| ATOM | 861 | CE1 | HIS | A | 108 | 23.063 | 5.470 | 12.862 | 1.00 | 10.00 | A | C |
| ATOM | 862 | NE2 | HIS | A | 108 | 24.084 | 4.838 | 12.273 | 1.00 | 10.00 | A | N |
| ATOM | 864 | C | HIS | A | 108 | 20.699 | 4.473 | 9.098 | 1.00 | 10.00 | A | C |
| ATOM | 865 | O | HIS | A | 108 | 20.932 | 3.349 | 9.541 | 1.00 | 10.00 | A | O |
| ATOM | 866 | N | GLY | A | 109 | 19.491 | 5.017 | 9.106 | 1.00 | 10.00 | A | N |
| ATOM | 868 | CA | GLY | A | 109 | 18.353 | 4.313 | 9.666 | 1.00 | 10.00 | A | C |
| ATOM | 869 | C | GLY | A | 109 | 17.935 | 3.113 | 8.837 | 1.00 | 10.00 | A | C |
| ATOM | 870 | O | GLY | A | 109 | 17.701 | 2.036 | 9.372 | 1.00 | 10.00 | A | O |
| ATOM | 871 | N | VAL | A | 110 | 17.846 | 3.291 | 7.522 | 1.00 | 10.00 | A | N |
| ATOM | 873 | CA | VAL | A | 110 | 17.452 | 2.199 | 6.635 | 1.00 | 10.00 | A | C |
| ATOM | 874 | CB | VAL | A | 110 | 17.191 | 2.684 | 5.189 | 1.00 | 10.00 | A | C |
| ATOM | 875 | CG1 | VAL | A | 110 | 18.477 | 3.130 | 4.509 | 1.00 | 10.00 | A | C |
| ATOM | 876 | CG2 | VAL | A | 110 | 16.477 | 1.618 | 4.368 | 1.00 | 10.00 | A | C |
| ATOM | 877 | C | VAL | A | 110 | 18.504 | 1.087 | 6.646 | 1.00 | 10.00 | A | C |
| ATOM | 878 | O | VAL | A | 110 | 18.198 | −0.078 | 6.383 | 1.00 | 10.00 | A | O |
| ATOM | 879 | N | ARG | A | 111 | 19.736 | 1.459 | 6.980 | 1.00 | 10.00 | A | N |
| ATOM | 881 | CA | ARG | A | 111 | 20.845 | 0.518 | 7.037 | 1.00 | 10.00 | A | C |
| ATOM | 882 | CB | ARG | A | 111 | 22.155 | 1.259 | 7.329 | 1.00 | 10.00 | A | C |
| ATOM | 883 | CG | ARG | A | 111 | 23.394 | 0.387 | 7.243 | 1.00 | 10.00 | A | C |
| ATOM | 884 | CD | ARG | A | 111 | 24.472 | 0.849 | 8.211 | 1.00 | 10.00 | A | C |
| ATOM | 885 | NE | ARG | A | 111 | 25.349 | 1.869 | 7.634 | 1.00 | 10.00 | A | N |
| ATOM | 887 | CZ | ARG | A | 111 | 26.421 | 1.593 | 6.892 | 1.00 | 10.00 | A | C |
| ATOM | 888 | NH1 | ARG | A | 111 | 26.750 | 0.334 | 6.631 | 1.00 | 10.00 | A | N |
| ATOM | 891 | NH2 | ARG | A | 111 | 27.184 | 2.576 | 6.438 | 1.00 | 10.00 | A | N |
| ATOM | 894 | C | ARG | A | 111 | 20.602 | −0.558 | 8.096 | 1.00 | 10.00 | A | C |
| ATOM | 895 | O | ARG | A | 111 | 21.048 | −1.691 | 7.942 | 1.00 | 10.00 | A | O |
| ATOM | 896 | N | ASP | A | 112 | 19.885 | −0.202 | 9.159 | 1.00 | 10.00 | A | N |
| ATOM | 898 | CA | ASP | A | 112 | 19.597 | −1.143 | 10.241 | 1.00 | 10.00 | A | C |
| ATOM | 899 | CB | ASP | A | 112 | 19.131 | −0.406 | 11.503 | 1.00 | 10.00 | A | C |
| ATOM | 900 | CG | ASP | A | 112 | 17.665 | −0.642 | 11.835 | 1.00 | 10.00 | A | C |
| ATOM | 901 | OD1 | ASP | A | 112 | 17.371 | −1.523 | 12.665 | 1.00 | 10.00 | A | O |
| ATOM | 902 | OD2 | ASP | A | 112 | 16.802 | 0.059 | 11.266 | 1.00 | 10.00 | A | O |
| ATOM | 903 | C | ASP | A | 112 | 18.578 | −2.204 | 9.823 | 1.00 | 10.00 | A | C |
| ATOM | 904 | O | ASP | A | 112 | 18.645 | −3.350 | 10.274 | 1.00 | 10.00 | A | O |
| ATOM | 905 | N | MET | A | 113 | 17.638 | −1.821 | 8.962 | 1.00 | 10.00 | A | N |
| ATOM | 907 | CA | MET | A | 113 | 16.619 | −2.754 | 8.495 | 1.00 | 10.00 | A | C |
| ATOM | 908 | CB | MET | A | 113 | 15.412 | −2.019 | 7.929 | 1.00 | 10.00 | A | C |
| ATOM | 909 | CG | MET | A | 113 | 14.094 | −2.719 | 8.217 | 1.00 | 10.00 | A | C |
| ATOM | 910 | SD | MET | A | 113 | 13.528 | −2.467 | 9.911 | 1.00 | 10.00 | A | S |
| ATOM | 911 | CE | MET | A | 113 | 12.337 | −3.796 | 10.065 | 1.00 | 10.00 | A | C |
| ATOM | 912 | C | MET | A | 113 | 17.193 | −3.695 | 7.456 | 1.00 | 10.00 | A | C |
| ATOM | 913 | O | MET | A | 113 | 16.768 | −4.846 | 7.332 | 1.00 | 10.00 | A | O |
| ATOM | 914 | N | ALA | A | 114 | 18.138 | −3.181 | 6.686 | 1.00 | 10.00 | A | N |
| ATOM | 916 | CA | ALA | A | 114 | 18.808 | −3.978 | 5.678 | 1.00 | 10.00 | A | C |
| ATOM | 917 | CB | ALA | A | 114 | 19.553 | −3.073 | 4.709 | 1.00 | 10.00 | A | C |
| ATOM | 918 | C | ALA | A | 114 | 19.787 | −4.891 | 6.392 | 1.00 | 10.00 | A | C |
| ATOM | 919 | O | ALA | A | 114 | 19.788 | −6.106 | 6.189 | 1.00 | 10.00 | A | O |
| ATOM | 920 | N | ALA | A | 115 | 20.587 | −4.268 | 7.259 | 1.00 | 10.00 | A | N |
| ATOM | 922 | CA | ALA | A | 115 | 21.589 | −4.942 | 8.072 | 1.00 | 10.00 | A | C |
| ATOM | 923 | CB | ALA | A | 115 | 20.963 | −5.605 | 9.287 | 1.00 | 10.00 | A | C |
| ATOM | 924 | C | ALA | A | 115 | 22.457 | −5.910 | 7.281 | 1.00 | 10.00 | A | C |
| ATOM | 925 | O | ALA | A | 115 | 22.152 | −7.100 | 7.172 | 1.00 | 10.00 | A | O |
| ATOM | 926 | N | ILE | A | 116 | 23.564 | −5.400 | 6.754 | 1.00 | 10.00 | A | N |
| ATOM | 928 | CA | ILE | A | 116 | 24.490 | −6.212 | 5.978 | 1.00 | 10.00 | A | C |
| ATOM | 929 | CB | ILE | A | 116 | 25.621 | −5.365 | 5.349 | 1.00 | 10.00 | A | C |
| ATOM | 930 | CG1 | ILE | A | 116 | 26.214 | −6.078 | 4.129 | 1.00 | 10.00 | A | C |
| ATOM | 931 | CG2 | ILE | A | 116 | 26.705 | −5.036 | 6.373 | 1.00 | 10.00 | A | C |
| ATOM | 932 | CD1 | ILE | A | 116 | 27.156 | −5.222 | 3.308 | 1.00 | 10.00 | A | C |
| ATOM | 933 | C | ILE | A | 116 | 25.071 | −7.349 | 6.821 | 1.00 | 10.00 | A | C |
| ATOM | 934 | O | ILE | A | 116 | 25.669 | −8.281 | 6.284 | 1.00 | 10.00 | A | O |
| ATOM | 935 | N | ARG | A | 117 | 24.870 | −7.263 | 8.145 | 1.00 | 10.00 | A | N |
| ATOM | 937 | CA | ARG | A | 117 | 25.343 | −8.276 | 9.093 | 1.00 | 10.00 | A | C |
| ATOM | 938 | CB | ARG | A | 117 | 24.713 | −8.037 | 10.472 | 1.00 | 10.00 | A | C |
| ATOM | 939 | CG | ARG | A | 117 | 25.205 | −8.982 | 11.559 | 1.00 | 10.00 | A | C |
| ATOM | 940 | CD | ARG | A | 117 | 24.227 | −9.047 | 12.724 | 1.00 | 10.00 | A | C |
| ATOM | 941 | NE | ARG | A | 117 | 24.802 | −9.728 | 13.878 | 1.00 | 10.00 | A | N |
| ATOM | 943 | CZ | ARG | A | 117 | 24.209 | −10.728 | 14.537 | 1.00 | 10.00 | A | C |
| ATOM | 944 | NH1 | ARG | A | 117 | 23.015 | −11.171 | 14.158 | 1.00 | 10.00 | A | N |
| ATOM | 947 | NH2 | ARG | A | 117 | 24.814 | −11.286 | 15.577 | 1.00 | 10.00 | A | N |
| ATOM | 950 | C | ARG | A | 117 | 24.992 | −9.680 | 8.607 | 1.00 | 10.00 | A | C |
| ATOM | 951 | O | ARG | A | 117 | 25.844 | −10.568 | 8.577 | 1.00 | 10.00 | A | O |
| ATOM | 952 | N | GLN | A | 118 | 23.740 | −9.868 | 8.216 | 1.00 | 10.00 | A | N |
| ATOM | 954 | CA | GLN | A | 118 | 23.285 | −11.159 | 7.723 | 1.00 | 10.00 | A | C |
| ATOM | 955 | CB | GLN | A | 118 | 22.405 | −11.881 | 8.744 | 1.00 | 10.00 | A | C |
| ATOM | 956 | CG | GLN | A | 118 | 23.152 | −12.408 | 9.959 | 1.00 | 10.00 | A | C |
| ATOM | 957 | CD | GLN | A | 118 | 22.234 | −13.094 | 10.947 | 1.00 | 10.00 | A | C |
| ATOM | 958 | OE1 | GLN | A | 118 | 21.671 | −12.453 | 11.830 | 1.00 | 10.00 | A | O |
| ATOM | 959 | NE2 | GLN | A | 118 | 22.093 | −14.403 | 10.817 | 1.00 | 10.00 | A | N |
| ATOM | 962 | C | GLN | A | 118 | 22.565 | −11.013 | 6.388 | 1.00 | 10.00 | A | C |
| ATOM | 963 | O | GLN | A | 118 | 21.978 | −11.973 | 5.891 | 1.00 | 10.00 | A | O |
| ATOM | 964 | N | LEU | A | 119 | 22.566 | −9.796 | 5.843 | 1.00 | 10.00 | A | N |
| ATOM | 966 | CA | LEU | A | 119 | 21.961 | −9.547 | 4.538 | 1.00 | 10.00 | A | C |
| ATOM | 967 | CB | LEU | A | 119 | 22.110 | −8.064 | 4.172 | 1.00 | 10.00 | A | C |
| ATOM | 968 | CG | LEU | A | 119 | 21.585 | −7.632 | 2.801 | 1.00 | 10.00 | A | C |
| ATOM | 969 | CD1 | LEU | A | 119 | 20.149 | −7.145 | 2.899 | 1.00 | 10.00 | A | C |
| ATOM | 970 | CD2 | LEU | A | 119 | 22.483 | −6.566 | 2.189 | 1.00 | 10.00 | A | C |
| ATOM | 971 | C | LEU | A | 119 | 22.726 | −10.398 | 3.540 | 1.00 | 10.00 | A | C |
| ATOM | 972 | O | LEU | A | 119 | 22.155 | −11.173 | 2.769 | 1.00 | 10.00 | A | O |
| ATOM | 973 | N | LYS | A | 120 | 24.044 | −10.258 | 3.597 | 1.00 | 10.00 | A | N |
| ATOM | 975 | CA | LYS | A | 120 | 24.927 | −11.030 | 2.760 | 1.00 | 10.00 | A | C |
| ATOM | 976 | CB | LYS | A | 120 | 26.016 | −10.154 | 2.145 | 1.00 | 10.00 | A | C |
| ATOM | 977 | CG | LYS | A | 120 | 25.486 | −9.191 | 1.092 | 1.00 | 10.00 | A | C |
| ATOM | 978 | CD | LYS | A | 120 | 24.624 | −9.913 | 0.063 | 1.00 | 10.00 | A | C |
| ATOM | 979 | CE | LYS | A | 120 | 23.926 | −8.929 | −0.865 | 1.00 | 10.00 | A | C |
| ATOM | 980 | NZ | LYS | A | 120 | 22.813 | −9.565 | −1.621 | 1.00 | 10.00 | A | N |
| ATOM | 984 | C | LYS | A | 120 | 25.505 | −12.154 | 3.605 | 1.00 | 10.00 | A | C |
| ATOM | 985 | O | LYS | A | 120 | 24.847 | −12.590 | 4.549 | 1.00 | 10.00 | A | O |
| ATOM | 986 | N | ALA | A | 121 | 26.713 | −12.611 | 3.279 | 1.00 | 10.00 | A | N |
| ATOM | 988 | CA | ALA | A | 121 | 27.366 | −13.702 | 4.011 | 1.00 | 10.00 | A | C |
| ATOM | 989 | CB | ALA | A | 121 | 27.541 | −13.375 | 5.490 | 1.00 | 10.00 | A | C |
| ATOM | 990 | C | ALA | A | 121 | 26.604 | −15.008 | 3.812 | 1.00 | 10.00 | A | C |
| ATOM | 991 | O | ALA | A | 121 | 27.058 | −15.895 | 3.092 | 1.00 | 10.00 | A | O |
| ATOM | 992 | N | THR | A | 122 | 25.440 | −15.107 | 4.437 | 1.00 | 10.00 | A | N |
| ATOM | 994 | CA | THR | A | 122 | 24.588 | −16.273 | 4.316 | 1.00 | 10.00 | A | C |
| ATOM | 995 | CB | THR | A | 122 | 23.529 | −16.275 | 5.434 | 1.00 | 10.00 | A | C |
| ATOM | 996 | OG1 | THR | A | 122 | 23.991 | −15.467 | 6.530 | 1.00 | 10.00 | A | O |
| ATOM | 998 | CG2 | THR | A | 122 | 23.278 | −17.690 | 5.925 | 1.00 | 10.00 | A | C |
| ATOM | 999 | C | THR | A | 122 | 23.887 | −16.229 | 2.961 | 1.00 | 10.00 | A | C |
| ATOM | 1000 | O | THR | A | 122 | 23.399 | −17.242 | 2.457 | 1.00 | 10.00 | A | O |
| ATOM | 1001 | N | HIS | A | 123 | 23.856 | −15.024 | 2.386 | 1.00 | 10.00 | A | N |
| ATOM | 1003 | CA | HIS | A | 123 | 23.247 | −14.772 | 1.081 | 1.00 | 10.00 | A | C |
| ATOM | 1004 | CB | HIS | A | 123 | 24.045 | −15.415 | −0.059 | 1.00 | 10.00 | A | C |
| ATOM | 1005 | CG | HIS | A | 123 | 25.361 | −14.748 | −0.323 | 1.00 | 10.00 | A | C |
| ATOM | 1006 | ND1 | HIS | A | 123 | 25.537 | −13.397 | −0.115 | 1.00 | 10.00 | A | N |
| ATOM | 1007 | CD2 | HIS | A | 123 | 26.519 | −15.283 | −0.775 | 1.00 | 10.00 | A | C |
| ATOM | 1008 | CE1 | HIS | A | 123 | 26.790 | −13.144 | −0.443 | 1.00 | 10.00 | A | C |
| ATOM | 1009 | NE2 | HIS | A | 123 | 27.420 | −14.252 | −0.848 | 1.00 | 10.00 | A | N |
| ATOM | 1011 | C | HIS | A | 123 | 21.782 | −15.180 | 1.036 | 1.00 | 10.00 | A | C |
| ATOM | 1012 | O | HIS | A | 123 | 21.365 | −15.973 | 0.191 | 1.00 | 10.00 | A | O |
| ATOM | 1013 | N | THR | A | 124 | 21.007 | −14.629 | 1.953 | 1.00 | 10.00 | A | N |
| ATOM | 1015 | CA | THR | A | 124 | 19.586 | −14.927 | 2.027 | 1.00 | 10.00 | A | C |
| ATOM | 1016 | CB | THR | A | 124 | 19.332 | −16.351 | 2.592 | 1.00 | 10.00 | A | C |
| ATOM | 1017 | OG1 | THR | A | 124 | 17.928 | −16.649 | 2.624 | 1.00 | 10.00 | A | O |
| ATOM | 1019 | CG2 | THR | A | 124 | 19.913 | −16.496 | 3.989 | 1.00 | 10.00 | A | C |
| ATOM | 1020 | C | THR | A | 124 | 18.864 | −13.880 | 2.872 | 1.00 | 10.00 | A | C |
| ATOM | 1021 | O | THR | A | 124 | 17.702 | −14.061 | 3.225 | 1.00 | 10.00 | A | O |
| ATOM | 1022 | N | ASP | A | 125 | 19.561 | −12.776 | 3.172 | 1.00 | 10.00 | A | N |
| ATOM | 1024 | CA | ASP | A | 125 | 18.996 | −11.694 | 3.974 | 1.00 | 10.00 | A | C |
| ATOM | 1025 | CB | ASP | A | 125 | 17.919 | −10.935 | 3.180 | 1.00 | 10.00 | A | C |
| ATOM | 1026 | CG | ASP | A | 125 | 18.379 | −10.486 | 1.802 | 1.00 | 10.00 | A | C |
| ATOM | 1027 | OD1 | ASP | A | 125 | 19.584 | −10.202 | 1.625 | 1.00 | 10.00 | A | O |
| ATOM | 1028 | OD2 | ASP | A | 125 | 17.535 | −10.423 | 0.886 | 1.00 | 10.00 | A | O |
| ATOM | 1029 | C | ASP | A | 125 | 18.416 | −12.238 | 5.285 | 1.00 | 10.00 | A | C |
| ATOM | 1030 | O | ASP | A | 125 | 17.245 | −12.018 | 5.599 | 1.00 | 10.00 | A | O |
| ATOM | 1031 | N | SER | A | 126 | 19.252 | −12.940 | 6.047 | 1.00 | 10.00 | A | N |
| ATOM | 1033 | CA | SER | A | 126 | 18.838 | −13.554 | 7.307 | 1.00 | 10.00 | A | C |
| ATOM | 1034 | CB | SER | A | 126 | 19.973 | −14.415 | 7.873 | 1.00 | 10.00 | A | C |
| ATOM | 1035 | OG | SER | A | 126 | 20.555 | −15.221 | 6.861 | 1.00 | 10.00 | A | O |
| ATOM | 1037 | C | SER | A | 126 | 18.351 | −12.542 | 8.350 | 1.00 | 10.00 | A | C |
| ATOM | 1038 | O | SER | A | 126 | 17.226 | −12.645 | 8.841 | 1.00 | 10.00 | A | O |
| ATOM | 1039 | N | VAL | A | 127 | 19.185 | −11.559 | 8.672 | 1.00 | 10.00 | A | N |
| ATOM | 1041 | CA | VAL | A | 127 | 18.828 | −10.554 | 9.672 | 1.00 | 10.00 | A | C |
| ATOM | 1042 | CB | VAL | A | 127 | 20.081 | −9.876 | 10.272 | 1.00 | 10.00 | A | C |
| ATOM | 1043 | CG1 | VAL | A | 127 | 20.721 | −8.927 | 9.277 | 1.00 | 10.00 | A | C |
| ATOM | 1044 | CG2 | VAL | A | 127 | 19.762 | −9.167 | 11.580 | 1.00 | 10.00 | A | C |
| ATOM | 1045 | C | VAL | A | 127 | 17.870 | −9.514 | 9.093 | 1.00 | 10.00 | A | C |
| ATOM | 1046 | O | VAL | A | 127 | 17.193 | −8.792 | 9.826 | 1.00 | 10.00 | A | O |
| ATOM | 1047 | N | SER | A | 128 | 17.814 | −9.442 | 7.773 | 1.00 | 10.00 | A | N |
| ATOM | 1049 | CA | SER | A | 128 | 16.925 | −8.513 | 7.113 | 1.00 | 10.00 | A | C |
| ATOM | 1050 | CB | SER | A | 128 | 17.289 | −8.438 | 5.639 | 1.00 | 10.00 | A | C |
| ATOM | 1051 | OG | SER | A | 128 | 18.695 | −8.563 | 5.490 | 1.00 | 10.00 | A | O |
| ATOM | 1053 | C | SER | A | 128 | 15.486 | −8.979 | 7.300 | 1.00 | 10.00 | A | C |
| ATOM | 1054 | O | SER | A | 128 | 14.575 | −8.164 | 7.460 | 1.00 | 10.00 | A | O |
| ATOM | 1055 | N | SER | A | 129 | 15.305 | −10.298 | 7.302 | 1.00 | 10.00 | A | N |
| ATOM | 1057 | CA | SER | A | 129 | 13.995 | −10.900 | 7.490 | 1.00 | 10.00 | A | C |
| ATOM | 1058 | CB | SER | A | 129 | 13.919 | −12.230 | 6.730 | 1.00 | 10.00 | A | C |
| ATOM | 1059 | OG | SER | A | 129 | 15.120 | −12.975 | 6.880 | 1.00 | 10.00 | A | O |
| ATOM | 1061 | C | SER | A | 129 | 13.735 | −11.116 | 8.977 | 1.00 | 10.00 | A | C |
| ATOM | 1062 | O | SER | A | 129 | 12.710 | −10.694 | 9.511 | 1.00 | 10.00 | A | O |
| ATOM | 1063 | N | GLU | A | 130 | 14.679 | −11.776 | 9.634 | 1.00 | 10.00 | A | N |
| ATOM | 1065 | CA | GLU | A | 130 | 14.593 | −12.041 | 11.057 | 1.00 | 10.00 | A | C |
| ATOM | 1066 | CB | GLU | A | 130 | 15.136 | −13.434 | 11.375 | 1.00 | 10.00 | A | C |
| ATOM | 1067 | CG | GLU | A | 130 | 14.269 | −14.566 | 10.858 | 1.00 | 10.00 | A | C |
| ATOM | 1068 | CD | GLU | A | 130 | 12.863 | −14.507 | 11.407 | 1.00 | 10.00 | A | C |
| ATOM | 1069 | OE1 | GLU | A | 130 | 12.701 | −14.467 | 12.644 | 1.00 | 10.00 | A | O |
| ATOM | 1070 | OE2 | GLU | A | 130 | 11.909 | −14.511 | 10.607 | 1.00 | 10.00 | A | O |
| ATOM | 1071 | C | GLU | A | 130 | 15.384 | −10.983 | 11.804 | 1.00 | 10.00 | A | C |
| ATOM | 1072 | O | GLU | A | 130 | 16.613 | −10.962 | 11.749 | 1.00 | 10.00 | A | O |
| ATOM | 1073 | N | GLN | A | 131 | 14.662 | −10.117 | 12.499 | 1.00 | 10.00 | A | N |
| ATOM | 1075 | CA | GLN | A | 131 | 15.261 | −9.013 | 13.234 | 1.00 | 10.00 | A | C |
| ATOM | 1076 | CB | GLN | A | 131 | 14.202 | −8.262 | 14.039 | 1.00 | 10.00 | A | C |
| ATOM | 1077 | CG | GLN | A | 131 | 13.195 | −7.528 | 13.173 | 1.00 | 10.00 | A | C |
| ATOM | 1078 | CD | GLN | A | 131 | 12.418 | −6.478 | 13.936 | 1.00 | 10.00 | A | C |
| ATOM | 1079 | OE1 | GLN | A | 131 | 11.345 | −6.747 | 14.470 | 1.00 | 10.00 | A | O |
| ATOM | 1080 | NE2 | GLN | A | 131 | 12.970 | −5.280 | 14.011 | 1.00 | 10.00 | A | N |
| ATOM | 1083 | C | GLN | A | 131 | 16.423 | −9.432 | 14.125 | 1.00 | 10.00 | A | C |
| ATOM | 1084 | O | GLN | A | 131 | 17.518 | −8.893 | 13.996 | 1.00 | 10.00 | A | O |
| ATOM | 1085 | N | VAL | A | 132 | 16.178 | −10.396 | 15.016 | 1.00 | 10.00 | A | N |
| ATOM | 1087 | CA | VAL | A | 132 | 17.197 | −10.892 | 15.954 | 1.00 | 10.00 | A | C |
| ATOM | 1088 | CB | VAL | A | 132 | 18.071 | −12.057 | 15.416 | 1.00 | 10.00 | A | C |
| ATOM | 1089 | CG1 | VAL | A | 132 | 17.216 | −13.287 | 15.139 | 1.00 | 10.00 | A | C |
| ATOM | 1090 | CG2 | VAL | A | 132 | 18.866 | −11.666 | 14.179 | 1.00 | 10.00 | A | C |
| ATOM | 1091 | C | VAL | A | 132 | 18.033 | −9.772 | 16.587 | 1.00 | 10.00 | A | C |
| ATOM | 1092 | O | VAL | A | 132 | 19.267 | −9.789 | 16.550 | 1.00 | 10.00 | A | O |
| ATOM | 1093 | N | ASP | A | 133 | 17.322 | −8.791 | 17.145 | 1.00 | 10.00 | A | N |
| ATOM | 1095 | CA | ASP | A | 133 | 17.920 | −7.627 | 17.813 | 1.00 | 10.00 | A | C |
| ATOM | 1096 | CB | ASP | A | 133 | 18.984 | −8.040 | 18.838 | 1.00 | 10.00 | A | C |
| ATOM | 1097 | CG | ASP | A | 133 | 19.112 | −7.077 | 20.005 | 1.00 | 10.00 | A | C |
| ATOM | 1098 | OD1 | ASP | A | 133 | 18.121 | −6.394 | 20.341 | 1.00 | 10.00 | A | O |
| ATOM | 1099 | OD2 | ASP | A | 133 | 20.202 | −7.026 | 20.613 | 1.00 | 10.00 | A | O |
| ATOM | 1100 | C | ASP | A | 133 | 18.459 | −6.576 | 16.831 | 1.00 | 10.00 | A | C |
| ATOM | 1101 | O | ASP | A | 133 | 18.877 | −5.496 | 17.245 | 1.00 | 10.00 | A | O |
| ATOM | 1102 | N | ASN | A | 134 | 18.432 | −6.892 | 15.536 | 1.00 | 10.00 | A | N |
| ATOM | 1104 | CA | ASN | A | 134 | 18.907 | −5.984 | 14.480 | 1.00 | 10.00 | A | C |
| ATOM | 1105 | CB | ASN | A | 134 | 17.993 | −4.761 | 14.307 | 1.00 | 10.00 | A | C |
| ATOM | 1106 | CG | ASN | A | 134 | 16.755 | −5.060 | 13.487 | 1.00 | 10.00 | A | C |
| ATOM | 1107 | OD1 | ASN | A | 134 | 15.751 | −5.529 | 14.018 | 1.00 | 10.00 | A | O |
| ATOM | 1108 | ND2 | ASN | A | 134 | 16.810 | −4.772 | 12.194 | 1.00 | 10.00 | A | N |
| ATOM | 1111 | C | ASN | A | 134 | 20.349 | −5.544 | 14.705 | 1.00 | 10.00 | A | C |
| ATOM | 1112 | O | ASN | A | 134 | 21.287 | −6.307 | 14.477 | 1.00 | 10.00 | A | O |
| ATOM | 1113 | N | VAL | A | 135 | 20.511 | −4.316 | 15.181 | 1.00 | 10.00 | A | N |
| ATOM | 1115 | CA | VAL | A | 135 | 21.826 | −3.747 | 15.447 | 1.00 | 10.00 | A | C |
| ATOM | 1116 | CB | VAL | A | 135 | 22.000 | −2.383 | 14.744 | 1.00 | 10.00 | A | C |
| ATOM | 1117 | CG1 | VAL | A | 135 | 22.183 | −2.589 | 13.248 | 1.00 | 10.00 | A | C |
| ATOM | 1118 | CG2 | VAL | A | 135 | 20.797 | −1.485 | 15.004 | 1.00 | 10.00 | A | C |
| ATOM | 1119 | C | VAL | A | 135 | 22.050 | −3.599 | 16.952 | 1.00 | 10.00 | A | C |
| ATOM | 1120 | O | VAL | A | 135 | 22.635 | −2.623 | 17.424 | 1.00 | 10.00 | A | O |
| ATOM | 1121 | N | ARG | A | 136 | 21.565 | −4.587 | 17.692 | 1.00 | 10.00 | A | N |
| ATOM | 1123 | CA | ARG | A | 136 | 21.681 | −4.617 | 19.147 | 1.00 | 10.00 | A | C |
| ATOM | 1124 | CB | ARG | A | 136 | 23.148 | −4.660 | 19.610 | 1.00 | 10.00 | A | C |
| ATOM | 1125 | CG | ARG | A | 136 | 23.362 | −5.403 | 20.923 | 1.00 | 10.00 | A | C |
| ATOM | 1126 | CD | ARG | A | 136 | 24.734 | −5.122 | 21.515 | 1.00 | 10.00 | A | C |
| ATOM | 1127 | NE | ARG | A | 136 | 25.770 | −6.015 | 20.995 | 1.00 | 10.00 | A | N |
| ATOM | 1129 | CZ | ARG | A | 136 | 27.012 | −6.070 | 21.477 | 1.00 | 10.00 | A | C |
| ATOM | 1130 | NH1 | ARG | A | 136 | 27.372 | −5.285 | 22.481 | 1.00 | 10.00 | A | N |
| ATOM | 1133 | NH2 | ARG | A | 136 | 27.892 | −6.924 | 20.966 | 1.00 | 10.00 | A | N |
| ATOM | 1136 | C | ARG | A | 136 | 20.909 | −3.455 | 19.780 | 1.00 | 10.00 | A | C |
| ATOM | 1137 | O | ARG | A | 136 | 19.770 | −3.187 | 19.406 | 1.00 | 10.00 | A | O |
| ATOM | 1138 | N | ARG | A | 137 | 21.537 | −2.742 | 20.706 | 1.00 | 10.00 | A | N |
| ATOM | 1140 | CA | ARG | A | 137 | 20.884 | −1.631 | 21.388 | 1.00 | 10.00 | A | C |
| ATOM | 1141 | CB | ARG | A | 137 | 21.632 | −1.283 | 22.677 | 1.00 | 10.00 | A | C |
| ATOM | 1142 | CG | ARG | A | 137 | 20.849 | −0.412 | 23.647 | 1.00 | 10.00 | A | C |
| ATOM | 1143 | CD | ARG | A | 137 | 20.091 | −1.253 | 24.658 | 1.00 | 10.00 | A | C |
| ATOM | 1144 | NE | ARG | A | 137 | 18.955 | −1.958 | 24.067 | 1.00 | 10.00 | A | N |
| ATOM | 1146 | CZ | ARG | A | 137 | 17.706 | −1.842 | 24.511 | 1.00 | 10.00 | A | C |
| ATOM | 1147 | NH1 | ARG | A | 137 | 17.438 | −1.043 | 25.539 | 1.00 | 10.00 | A | N |
| ATOM | 1150 | NH2 | ARG | A | 137 | 16.725 | −2.519 | 23.927 | 1.00 | 10.00 | A | N |
| ATOM | 1153 | C | ARG | A | 137 | 20.775 | −0.407 | 20.487 | 1.00 | 10.00 | A | C |
| ATOM | 1154 | O | ARG | A | 137 | 20.005 | 0.515 | 20.766 | 1.00 | 10.00 | A | O |
| ATOM | 1155 | N | MET | A | 138 | 21.529 | −0.412 | 19.392 | 1.00 | 10.00 | A | N |
| ATOM | 1157 | CA | MET | A | 138 | 21.522 | 0.706 | 18.457 | 1.00 | 10.00 | A | C |
| ATOM | 1158 | CB | MET | A | 138 | 22.628 | 0.579 | 17.411 | 1.00 | 10.00 | A | C |
| ATOM | 1159 | CG | MET | A | 138 | 24.007 | 0.963 | 17.925 | 1.00 | 10.00 | A | C |
| ATOM | 1160 | SD | MET | A | 138 | 25.298 | 0.730 | 16.688 | 1.00 | 10.00 | A | S |
| ATOM | 1161 | CE | MET | A | 138 | 25.171 | −1.035 | 16.399 | 1.00 | 10.00 | A | C |
| ATOM | 1162 | C | MET | A | 138 | 20.159 | 0.875 | 17.796 | 1.00 | 10.00 | A | C |
| ATOM | 1163 | O | MET | A | 138 | 19.861 | 1.930 | 17.240 | 1.00 | 10.00 | A | O |
| ATOM | 1164 | N | LEU | A | 139 | 19.328 | −0.162 | 17.882 | 1.00 | 10.00 | A | N |
| ATOM | 1166 | CA | LEU | A | 139 | 17.983 | −0.125 | 17.316 | 1.00 | 10.00 | A | C |
| ATOM | 1167 | CB | LEU | A | 139 | 17.271 | −1.464 | 17.558 | 1.00 | 10.00 | A | C |
| ATOM | 1168 | CG | LEU | A | 139 | 15.858 | −1.594 | 16.987 | 1.00 | 10.00 | A | C |
| ATOM | 1169 | CD1 | LEU | A | 139 | 15.860 | −1.328 | 15.492 | 1.00 | 10.00 | A | C |
| ATOM | 1170 | CD2 | LEU | A | 139 | 15.284 | −2.969 | 17.279 | 1.00 | 10.00 | A | C |
| ATOM | 1171 | C | LEU | A | 139 | 17.190 | 1.021 | 17.941 | 1.00 | 10.00 | A | C |
| ATOM | 1172 | O | LEU | A | 139 | 16.295 | 1.594 | 17.321 | 1.00 | 10.00 | A | O |
| ATOM | 1173 | N | LEU | A | 140 | 17.548 | 1.362 | 19.173 | 1.00 | 10.00 | A | N |
| ATOM | 1175 | CA | LEU | A | 140 | 16.896 | 2.441 | 19.889 | 1.00 | 10.00 | A | C |
| ATOM | 1176 | CB | LEU | A | 140 | 16.332 | 1.948 | 21.226 | 1.00 | 10.00 | A | C |
| ATOM | 1177 | CG | LEU | A | 140 | 15.100 | 1.042 | 21.149 | 1.00 | 10.00 | A | C |
| ATOM | 1178 | CD1 | LEU | A | 140 | 14.742 | 0.505 | 22.526 | 1.00 | 10.00 | A | C |
| ATOM | 1179 | CD2 | LEU | A | 140 | 13.924 | 1.788 | 20.542 | 1.00 | 10.00 | A | C |
| ATOM | 1180 | C | LEU | A | 140 | 17.876 | 3.585 | 20.120 | 1.00 | 10.00 | A | C |
| ATOM | 1181 | O | LEU | A | 140 | 17.773 | 4.313 | 21.105 | 1.00 | 10.00 | A | O |
| ATOM | 1182 | N | ALA | A | 141 | 18.840 | 3.725 | 19.217 | 1.00 | 10.00 | A | N |
| ATOM | 1184 | CA | ALA | A | 141 | 19.837 | 4.781 | 19.323 | 1.00 | 10.00 | A | C |
| ATOM | 1185 | CB | ALA | A | 141 | 21.199 | 4.193 | 19.654 | 1.00 | 10.00 | A | C |
| ATOM | 1186 | C | ALA | A | 141 | 19.907 | 5.600 | 18.036 | 1.00 | 10.00 | A | C |
| ATOM | 1187 | O | ALA | A | 141 | 20.605 | 6.612 | 17.968 | 1.00 | 10.00 | A | O |
| ATOM | 1188 | N | MET | A | 142 | 19.183 | 5.158 | 17.018 | 1.00 | 10.00 | A | N |
| ATOM | 1190 | CA | MET | A | 142 | 19.162 | 5.851 | 15.732 | 1.00 | 10.00 | A | C |
| ATOM | 1191 | CB | MET | A | 142 | 19.156 | 4.841 | 14.584 | 1.00 | 10.00 | A | C |
| ATOM | 1192 | CG | MET | A | 142 | 20.271 | 3.813 | 14.648 | 1.00 | 10.00 | A | C |
| ATOM | 1193 | SD | MET | A | 142 | 19.947 | 2.385 | 13.599 | 1.00 | 10.00 | A | S |
| ATOM | 1194 | CE | MET | A | 142 | 18.347 | 1.870 | 14.221 | 1.00 | 10.00 | A | C |
| ATOM | 1195 | C | MET | A | 142 | 17.935 | 6.745 | 15.627 | 1.00 | 10.00 | A | C |
| ATOM | 1196 | O | MET | A | 142 | 16.938 | 6.517 | 16.313 | 1.00 | 10.00 | A | O |
| ATOM | 1197 | N | VAL | A | 143 | 18.013 | 7.753 | 14.765 | 1.00 | 10.00 | A | N |
| ATOM | 1199 | CA | VAL | A | 143 | 16.906 | 8.682 | 14.568 | 1.00 | 10.00 | A | C |
| ATOM | 1200 | CB | VAL | A | 143 | 17.326 | 9.907 | 13.726 | 1.00 | 10.00 | A | C |
| ATOM | 1201 | CG1 | VAL | A | 143 | 16.197 | 10.924 | 13.661 | 1.00 | 10.00 | A | C |
| ATOM | 1202 | CG2 | VAL | A | 143 | 18.588 | 10.546 | 14.288 | 1.00 | 10.00 | A | C |
| ATOM | 1203 | C | VAL | A | 143 | 15.735 | 7.974 | 13.891 | 1.00 | 10.00 | A | C |
| ATOM | 1204 | O | VAL | A | 143 | 15.825 | 7.585 | 12.725 | 1.00 | 10.00 | A | O |
| ATOM | 1205 | N | ASP | A | 144 | 14.635 | 7.827 | 14.620 | 1.00 | 10.00 | A | N |
| ATOM | 1207 | CA | ASP | A | 144 | 13.446 | 7.147 | 14.106 | 1.00 | 10.00 | A | C |
| ATOM | 1208 | CB | ASP | A | 144 | 12.635 | 6.515 | 15.250 | 1.00 | 10.00 | A | C |
| ATOM | 1209 | CG | ASP | A | 144 | 11.858 | 7.521 | 16.082 | 1.00 | 10.00 | A | C |
| ATOM | 1210 | OD1 | ASP | A | 144 | 12.470 | 8.489 | 16.576 | 1.00 | 10.00 | A | O |
| ATOM | 1211 | OD2 | ASP | A | 144 | 10.632 | 7.340 | 16.260 | 1.00 | 10.00 | A | O |
| ATOM | 1212 | C | ASP | A | 144 | 12.565 | 8.066 | 13.252 | 1.00 | 10.00 | A | C |
| ATOM | 1213 | O | ASP | A | 144 | 11.337 | 8.007 | 13.315 | 1.00 | 10.00 | A | O |
| ATOM | 1214 | N | ASP | A | 145 | 13.199 | 8.908 | 12.441 | 1.00 | 10.00 | A | N |
| ATOM | 1216 | CA | ASP | A | 145 | 12.472 | 9.824 | 11.567 | 1.00 | 10.00 | A | C |
| ATOM | 1217 | CB | ASP | A | 145 | 13.447 | 10.683 | 10.755 | 1.00 | 10.00 | A | C |
| ATOM | 1218 | CG | ASP | A | 145 | 12.753 | 11.802 | 10.001 | 1.00 | 10.00 | A | C |
| ATOM | 1219 | OD1 | ASP | A | 145 | 12.163 | 11.532 | 8.938 | 1.00 | 10.00 | A | O |
| ATOM | 1220 | OD2 | ASP | A | 145 | 12.791 | 12.960 | 10.474 | 1.00 | 10.00 | A | O |
| ATOM | 1221 | C | ASP | A | 145 | 11.562 | 9.038 | 10.631 | 1.00 | 10.00 | A | C |
| ATOM | 1222 | O | ASP | A | 145 | 11.951 | 7.983 | 10.122 | 1.00 | 10.00 | A | O |
| ATOM | 1223 | N | PHE | A | 146 | 10.355 | 9.549 | 10.423 | 1.00 | 10.00 | A | N |
| ATOM | 1225 | CA | PHE | A | 146 | 9.370 | 8.892 | 9.569 | 1.00 | 10.00 | A | C |
| ATOM | 1226 | CB | PHE | A | 146 | 8.057 | 9.692 | 9.508 | 1.00 | 10.00 | A | C |
| ATOM | 1227 | CG | PHE | A | 146 | 8.167 | 11.041 | 8.848 | 1.00 | 10.00 | A | C |
| ATOM | 1228 | CD1 | PHE | A | 146 | 8.760 | 12.106 | 9.500 | 1.00 | 10.00 | A | C |
| ATOM | 1229 | CD2 | PHE | A | 146 | 7.669 | 11.241 | 7.572 | 1.00 | 10.00 | A | C |
| ATOM | 1230 | CE1 | PHE | A | 146 | 8.855 | 13.343 | 8.894 | 1.00 | 10.00 | A | C |
| ATOM | 1231 | CE2 | PHE | A | 146 | 7.761 | 12.475 | 6.958 | 1.00 | 10.00 | A | C |
| ATOM | 1232 | CZ | PHE | A | 146 | 8.356 | 13.528 | 7.620 | 1.00 | 10.00 | A | C |
| ATOM | 1233 | C | PHE | A | 146 | 9.907 | 8.598 | 8.169 | 1.00 | 10.00 | A | C |
| ATOM | 1234 | O | PHE | A | 146 | 9.535 | 7.595 | 7.554 | 1.00 | 10.00 | A | O |
| ATOM | 1235 | N | ARG | A | 147 | 10.801 | 9.453 | 7.680 | 1.00 | 10.00 | A | N |
| ATOM | 1237 | CA | ARG | A | 147 | 11.382 | 9.276 | 6.356 | 1.00 | 10.00 | A | C |
| ATOM | 1238 | CB | ARG | A | 147 | 12.217 | 10.489 | 5.949 | 1.00 | 10.00 | A | C |
| ATOM | 1239 | CG | ARG | A | 147 | 11.391 | 11.686 | 5.507 | 1.00 | 10.00 | A | C |
| ATOM | 1240 | CD | ARG | A | 147 | 12.201 | 12.970 | 5.558 | 1.00 | 10.00 | A | C |
| ATOM | 1241 | NE | ARG | A | 147 | 12.582 | 13.318 | 6.926 | 1.00 | 10.00 | A | N |
| ATOM | 1243 | CZ | ARG | A | 147 | 13.165 | 14.467 | 7.272 | 1.00 | 10.00 | A | C |
| ATOM | 1244 | NH1 | ARG | A | 147 | 13.444 | 15.387 | 6.354 | 1.00 | 10.00 | A | N |
| ATOM | 1247 | NH2 | ARG | A | 147 | 13.459 | 14.699 | 8.545 | 1.00 | 10.00 | A | N |
| ATOM | 1250 | C | ARG | A | 147 | 12.203 | 7.994 | 6.273 | 1.00 | 10.00 | A | C |
| ATOM | 1251 | O | ARG | A | 147 | 12.281 | 7.372 | 5.215 | 1.00 | 10.00 | A | O |
| ATOM | 1252 | N | CYS | A | 148 | 12.792 | 7.589 | 7.397 | 1.00 | 10.00 | A | N |
| ATOM | 1254 | CA | CYS | A | 148 | 13.591 | 6.371 | 7.444 | 1.00 | 10.00 | A | C |
| ATOM | 1255 | CB | CYS | A | 148 | 14.331 | 6.260 | 8.779 | 1.00 | 10.00 | A | C |
| ATOM | 1256 | SG | CYS | A | 148 | 15.107 | 7.791 | 9.341 | 1.00 | 10.00 | A | S |
| ATOM | 1258 | C | CYS | A | 148 | 12.693 | 5.152 | 7.265 | 1.00 | 10.00 | A | C |
| ATOM | 1259 | O | CYS | A | 148 | 13.093 | 4.154 | 6.672 | 1.00 | 10.00 | A | O |
| ATOM | 1260 | N | VAL | A | 149 | 11.479 | 5.248 | 7.788 | 1.00 | 10.00 | A | N |
| ATOM | 1262 | CA | VAL | A | 149 | 10.518 | 4.158 | 7.691 | 1.00 | 10.00 | A | C |
| ATOM | 1263 | CB | VAL | A | 149 | 9.353 | 4.330 | 8.695 | 1.00 | 10.00 | A | C |
| ATOM | 1264 | CG1 | VAL | A | 149 | 8.429 | 3.120 | 8.669 | 1.00 | 10.00 | A | C |
| ATOM | 1265 | CG2 | VAL | A | 149 | 9.896 | 4.558 | 10.098 | 1.00 | 10.00 | A | C |
| ATOM | 1266 | C | VAL | A | 149 | 9.960 | 4.066 | 6.278 | 1.00 | 10.00 | A | C |
| ATOM | 1267 | O | VAL | A | 149 | 9.834 | 2.979 | 5.724 | 1.00 | 10.00 | A | O |
| ATOM | 1268 | N | VAL | A | 150 | 9.654 | 5.220 | 5.691 | 1.00 | 10.00 | A | N |
| ATOM | 1270 | CA | VAL | A | 150 | 9.100 | 5.277 | 4.338 | 1.00 | 10.00 | A | C |
| ATOM | 1271 | CB | VAL | A | 150 | 8.793 | 6.726 | 3.904 | 1.00 | 10.00 | A | C |
| ATOM | 1272 | CG1 | VAL | A | 150 | 8.206 | 6.765 | 2.500 | 1.00 | 10.00 | A | C |
| ATOM | 1273 | CG2 | VAL | A | 150 | 7.837 | 7.384 | 4.887 | 1.00 | 10.00 | A | C |
| ATOM | 1274 | C | VAL | A | 150 | 10.025 | 4.610 | 3.316 | 1.00 | 10.00 | A | C |
| ATOM | 1275 | O | VAL | A | 150 | 9.575 | 3.826 | 2.476 | 1.00 | 10.00 | A | O |
| ATOM | 1276 | N | ILE | A | 151 | 11.320 | 4.899 | 3.410 | 1.00 | 10.00 | A | N |
| ATOM | 1278 | CA | ILE | A | 151 | 12.290 | 4.314 | 2.484 | 1.00 | 10.00 | A | C |
| ATOM | 1279 | CB | ILE | A | 151 | 13.666 | 5.015 | 2.506 | 1.00 | 10.00 | A | C |
| ATOM | 1280 | CG1 | ILE | A | 151 | 14.219 | 5.105 | 3.931 | 1.00 | 10.00 | A | C |
| ATOM | 1281 | CG2 | ILE | A | 151 | 13.568 | 6.396 | 1.873 | 1.00 | 10.00 | A | C |
| ATOM | 1282 | CD1 | ILE | A | 151 | 15.605 | 5.705 | 4.012 | 1.00 | 10.00 | A | C |
| ATOM | 1283 | C | ILE | A | 151 | 12.438 | 2.813 | 2.708 | 1.00 | 10.00 | A | C |
| ATOM | 1284 | O | ILE | A | 151 | 12.654 | 2.058 | 1.761 | 1.00 | 10.00 | A | O |
| ATOM | 1285 | N | LYS | A | 152 | 12.313 | 2.387 | 3.966 | 1.00 | 10.00 | A | N |
| ATOM | 1287 | CA | LYS | A | 152 | 12.403 | 0.970 | 4.310 | 1.00 | 10.00 | A | C |
| ATOM | 1288 | CB | LYS | A | 152 | 12.294 | 0.779 | 5.827 | 1.00 | 10.00 | A | C |
| ATOM | 1289 | CG | LYS | A | 152 | 13.546 | 1.136 | 6.609 | 1.00 | 10.00 | A | C |
| ATOM | 1290 | CD | LYS | A | 152 | 13.317 | 0.969 | 8.104 | 1.00 | 10.00 | A | C |
| ATOM | 1291 | CE | LYS | A | 152 | 14.590 | 1.226 | 8.890 | 1.00 | 10.00 | A | C |
| ATOM | 1292 | NZ | LYS | A | 152 | 14.520 | 0.669 | 10.267 | 1.00 | 10.00 | A | N |
| ATOM | 1296 | C | LYS | A | 152 | 11.261 | 0.222 | 3.635 | 1.00 | 10.00 | A | C |
| ATOM | 1297 | O | LYS | A | 152 | 11.444 | −0.854 | 3.066 | 1.00 | 10.00 | A | O |
| ATOM | 1298 | N | LEU | A | 153 | 10.083 | 0.821 | 3.696 | 1.00 | 10.00 | A | N |
| ATOM | 1300 | CA | LEU | A | 153 | 8.894 | 0.246 | 3.095 | 1.00 | 10.00 | A | C |
| ATOM | 1301 | CB | LEU | A | 153 | 7.662 | 1.062 | 3.487 | 1.00 | 10.00 | A | C |
| ATOM | 1302 | CG | LEU | A | 153 | 7.404 | 1.232 | 4.986 | 1.00 | 10.00 | A | C |
| ATOM | 1303 | CD1 | LEU | A | 153 | 6.305 | 2.255 | 5.221 | 1.00 | 10.00 | A | C |
| ATOM | 1304 | CD2 | LEU | A | 153 | 7.044 | −0.101 | 5.625 | 1.00 | 10.00 | A | C |
| ATOM | 1305 | C | LEU | A | 153 | 9.025 | 0.214 | 1.580 | 1.00 | 10.00 | A | C |
| ATOM | 1306 | O | LEU | A | 153 | 8.818 | −0.828 | 0.959 | 1.00 | 10.00 | A | O |
| ATOM | 1307 | N | ALA | A | 154 | 9.395 | 1.354 | 0.998 | 1.00 | 10.00 | A | N |
| ATOM | 1309 | CA | ALA | A | 154 | 9.546 | 1.479 | −0.452 | 1.00 | 10.00 | A | C |
| ATOM | 1310 | CB | ALA | A | 154 | 9.998 | 2.881 | −0.828 | 1.00 | 10.00 | A | C |
| ATOM | 1311 | C | ALA | A | 154 | 10.513 | 0.446 | −1.014 | 1.00 | 10.00 | A | C |
| ATOM | 1312 | O | ALA | A | 154 | 10.233 | −0.184 | −2.035 | 1.00 | 10.00 | A | O |
| ATOM | 1313 | N | GLU | A | 155 | 11.643 | 0.274 | −0.341 | 1.00 | 10.00 | A | N |
| ATOM | 1315 | CA | GLU | A | 155 | 12.650 | −0.686 | −0.771 | 1.00 | 10.00 | A | C |
| ATOM | 1316 | CB | GLU | A | 155 | 13.867 | −0.636 | 0.160 | 1.00 | 10.00 | A | C |
| ATOM | 1317 | CG | GLU | A | 155 | 14.984 | −1.601 | −0.206 | 1.00 | 10.00 | A | C |
| ATOM | 1318 | CD | GLU | A | 155 | 15.083 | −2.770 | 0.753 | 1.00 | 10.00 | A | C |
| ATOM | 1319 | OE1 | GLU | A | 155 | 14.193 | −2.911 | 1.614 | 1.00 | 10.00 | A | O |
| ATOM | 1320 | OE2 | GLU | A | 155 | 16.053 | −3.549 | 0.651 | 1.00 | 10.00 | A | O |
| ATOM | 1321 | C | GLU | A | 155 | 12.065 | −2.096 | −0.826 | 1.00 | 10.00 | A | C |
| ATOM | 1322 | O | GLU | A | 155 | 12.236 | −2.817 | −1.813 | 1.00 | 10.00 | A | O |
| ATOM | 1323 | N | ARG | A | 156 | 11.341 | −2.462 | 0.224 | 1.00 | 10.00 | A | N |
| ATOM | 1325 | CA | ARG | A | 156 | 10.726 | −3.780 | 0.318 | 1.00 | 10.00 | A | C |
| ATOM | 1326 | CB | ARG | A | 156 | 10.215 | −4.048 | 1.729 | 1.00 | 10.00 | A | C |
| ATOM | 1327 | CG | ARG | A | 156 | 11.312 | −4.291 | 2.745 | 1.00 | 10.00 | A | C |
| ATOM | 1328 | CD | ARG | A | 156 | 12.292 | −5.347 | 2.264 | 1.00 | 10.00 | A | C |
| ATOM | 1329 | NE | ARG | A | 156 | 12.913 | −6.054 | 3.378 | 1.00 | 10.00 | A | N |
| ATOM | 1331 | CZ | ARG | A | 156 | 14.076 | −5.716 | 3.929 | 1.00 | 10.00 | A | C |
| ATOM | 1332 | NH1 | ARG | A | 156 | 14.758 | −4.666 | 3.484 | 1.00 | 10.00 | A | N |
| ATOM | 1335 | NH2 | ARG | A | 156 | 14.561 | −6.442 | 4.923 | 1.00 | 10.00 | A | N |
| ATOM | 1338 | C | ARG | A | 156 | 9.604 | −3.959 | −0.697 | 1.00 | 10.00 | A | C |
| ATOM | 1339 | O | ARG | A | 156 | 9.348 | −5.072 | −1.151 | 1.00 | 10.00 | A | O |
| ATOM | 1340 | N | ILE | A | 157 | 8.925 | −2.869 | −1.046 | 1.00 | 10.00 | A | N |
| ATOM | 1342 | CA | ILE | A | 157 | 7.845 | −2.934 | −2.029 | 1.00 | 10.00 | A | C |
| ATOM | 1343 | CB | ILE | A | 157 | 7.150 | −1.570 | −2.228 | 1.00 | 10.00 | A | C |
| ATOM | 1344 | CG1 | ILE | A | 157 | 6.483 | −1.115 | −0.928 | 1.00 | 10.00 | A | C |
| ATOM | 1345 | CG2 | ILE | A | 157 | 6.119 | −1.648 | −3.345 | 1.00 | 10.00 | A | C |
| ATOM | 1346 | CD1 | ILE | A | 157 | 6.057 | 0.335 | −0.935 | 1.00 | 10.00 | A | C |
| ATOM | 1347 | C | ILE | A | 157 | 8.405 | −3.423 | −3.361 | 1.00 | 10.00 | A | C |
| ATOM | 1348 | O | ILE | A | 157 | 7.835 | −4.302 | −4.004 | 1.00 | 10.00 | A | O |
| ATOM | 1349 | N | ALA | A | 158 | 9.540 | −2.856 | −3.752 | 1.00 | 10.00 | A | N |
| ATOM | 1351 | CA | ALA | A | 158 | 10.202 | −3.245 | −4.990 | 1.00 | 10.00 | A | C |
| ATOM | 1352 | CB | ALA | A | 158 | 11.376 | −2.319 | −5.274 | 1.00 | 10.00 | A | C |
| ATOM | 1353 | C | ALA | A | 158 | 10.684 | −4.691 | −4.893 | 1.00 | 10.00 | A | C |
| ATOM | 1354 | O | ALA | A | 158 | 10.638 | −5.442 | −5.866 | 1.00 | 10.00 | A | O |
| ATOM | 1355 | N | HIS | A | 159 | 11.130 | −5.072 | −3.702 | 1.00 | 10.00 | A | N |
| ATOM | 1357 | CA | HIS | A | 159 | 11.618 | −6.419 | −3.461 | 1.00 | 10.00 | A | C |
| ATOM | 1358 | CB | HIS | A | 159 | 12.306 | −6.502 | −2.097 | 1.00 | 10.00 | A | C |
| ATOM | 1359 | CG | HIS | A | 159 | 13.218 | −7.676 | −1.949 | 1.00 | 10.00 | A | C |
| ATOM | 1360 | ND1 | HIS | A | 159 | 12.806 | −8.822 | −1.320 | 1.00 | 10.00 | A | N |
| ATOM | 1361 | CD2 | HIS | A | 159 | 14.502 | −7.827 | −2.359 | 1.00 | 10.00 | A | C |
| ATOM | 1362 | CE1 | HIS | A | 159 | 13.836 | −9.644 | −1.356 | 1.00 | 10.00 | A | C |
| ATOM | 1363 | NE2 | HIS | A | 159 | 14.881 | −9.086 | −1.974 | 1.00 | 10.00 | A | N |
| ATOM | 1365 | C | HIS | A | 159 | 10.481 | −7.437 | −3.554 | 1.00 | 10.00 | A | C |
| ATOM | 1366 | O | HIS | A | 159 | 10.642 | −8.500 | −4.143 | 1.00 | 10.00 | A | O |
| ATOM | 1367 | N | LEU | A | 160 | 9.323 | −7.095 | −2.995 | 1.00 | 10.00 | A | N |
| ATOM | 1369 | CA | LEU | A | 160 | 8.163 | −7.985 | −3.025 | 1.00 | 10.00 | A | C |
| ATOM | 1370 | CB | LEU | A | 160 | 7.070 | −7.513 | −2.072 | 1.00 | 10.00 | A | C |
| ATOM | 1371 | CG | LEU | A | 160 | 7.189 | −7.995 | −0.627 | 1.00 | 10.00 | A | C |
| ATOM | 1372 | CD1 | LEU | A | 160 | 5.978 | −7.552 | 0.168 | 1.00 | 10.00 | A | C |
| ATOM | 1373 | CD2 | LEU | A | 160 | 7.339 | −9.510 | −0.573 | 1.00 | 10.00 | A | C |
| ATOM | 1374 | C | LEU | A | 160 | 7.620 | −8.150 | −4.439 | 1.00 | 10.00 | A | C |
| ATOM | 1375 | O | LEU | A | 160 | 6.891 | −9.100 | −4.736 | 1.00 | 10.00 | A | O |
| ATOM | 1376 | N | ARG | A | 161 | 7.967 | −7.213 | −5.304 | 1.00 | 10.00 | A | N |
| ATOM | 1378 | CA | ARG | A | 161 | 7.550 | −7.278 | −6.693 | 1.00 | 10.00 | A | C |
| ATOM | 1379 | CB | ARG | A | 161 | 7.406 | −5.879 | −7.288 | 1.00 | 10.00 | A | C |
| ATOM | 1380 | CG | ARG | A | 161 | 6.163 | −5.155 | −6.805 | 1.00 | 10.00 | A | C |
| ATOM | 1381 | CD | ARG | A | 161 | 6.217 | −3.673 | −7.120 | 1.00 | 10.00 | A | C |
| ATOM | 1382 | NE | ARG | A | 161 | 5.067 | −2.964 | −6.557 | 1.00 | 10.00 | A | N |
| ATOM | 1384 | CZ | ARG | A | 161 | 4.826 | −1.669 | −6.734 | 1.00 | 10.00 | A | C |
| ATOM | 1385 | NH1 | ARG | A | 161 | 5.656 | −0.928 | −7.461 | 1.00 | 10.00 | A | N |
| ATOM | 1388 | NH2 | ARG | A | 161 | 3.751 | −1.117 | −6.180 | 1.00 | 10.00 | A | N |
| ATOM | 1391 | C | ARG | A | 161 | 8.562 | −8.109 | −7.467 | 1.00 | 10.00 | A | C |
| ATOM | 1392 | O | ARG | A | 161 | 8.206 | −8.878 | −8.359 | 1.00 | 10.00 | A | O |
| ATOM | 1393 | N | GLU | A | 162 | 9.830 | −7.966 | −7.091 | 1.00 | 10.00 | A | N |
| ATOM | 1395 | CA | GLU | A | 162 | 10.906 | −8.721 | −7.720 | 1.00 | 10.00 | A | C |
| ATOM | 1396 | CB | GLU | A | 162 | 12.270 | −8.136 | −7.326 | 1.00 | 10.00 | A | C |
| ATOM | 1397 | CG | GLU | A | 162 | 13.452 | −8.701 | −8.105 | 1.00 | 10.00 | A | C |
| ATOM | 1398 | CD | GLU | A | 162 | 14.795 | −8.297 | −7.518 | 1.00 | 10.00 | A | C |
| ATOM | 1399 | OE1 | GLU | A | 162 | 14.954 | −7.120 | −7.134 | 1.00 | 10.00 | A | O |
| ATOM | 1400 | OE2 | GLU | A | 162 | 15.704 | −9.153 | −7.444 | 1.00 | 10.00 | A | O |
| ATOM | 1401 | C | GLU | A | 162 | 10.818 | −10.175 | −7.266 | 1.00 | 10.00 | A | C |
| ATOM | 1402 | O | GLU | A | 162 | 11.229 | −11.088 | −7.974 | 1.00 | 10.00 | A | O |
| ATOM | 1403 | N | VAL | A | 163 | 10.246 | −10.370 | −6.077 | 1.00 | 10.00 | A | N |
| ATOM | 1405 | CA | VAL | A | 163 | 10.088 | −11.694 | −5.478 | 1.00 | 10.00 | A | C |
| ATOM | 1406 | CB | VAL | A | 163 | 9.521 | −11.612 | −4.035 | 1.00 | 10.00 | A | C |
| ATOM | 1407 | CG1 | VAL | A | 163 | 8.031 | −11.936 | −3.976 | 1.00 | 10.00 | A | C |
| ATOM | 1408 | CG2 | VAL | A | 163 | 10.311 | −12.512 | −3.100 | 1.00 | 10.00 | A | C |
| ATOM | 1409 | C | VAL | A | 163 | 9.247 | −12.636 | −6.341 | 1.00 | 10.00 | A | C |
| ATOM | 1410 | O | VAL | A | 163 | 9.323 | −13.853 | −6.190 | 1.00 | 10.00 | A | O |
| ATOM | 1411 | N | LYS | A | 164 | 8.469 | −12.081 | −7.260 | 1.00 | 10.00 | A | N |
| ATOM | 1413 | CA | LYS | A | 164 | 7.636 | −12.898 | −8.134 | 1.00 | 10.00 | A | C |
| ATOM | 1414 | CB | LYS | A | 164 | 6.499 | −12.078 | −8.744 | 1.00 | 10.00 | A | C |
| ATOM | 1415 | CG | LYS | A | 164 | 6.902 | −11.211 | −9.924 | 1.00 | 10.00 | A | C |
| ATOM | 1416 | CD | LYS | A | 164 | 5.746 | −10.338 | −10.374 | 1.00 | 10.00 | A | C |
| ATOM | 1417 | CE | LYS | A | 164 | 6.035 | −9.686 | −11.713 | 1.00 | 10.00 | A | C |
| ATOM | 1418 | NZ | LYS | A | 164 | 4.887 | −8.868 | −12.185 | 1.00 | 10.00 | A | N |
| ATOM | 1422 | C | LYS | A | 164 | 8.478 | −13.550 | −9.224 | 1.00 | 10.00 | A | C |
| ATOM | 1423 | O | LYS | A | 164 | 8.023 | −14.459 | −9.917 | 1.00 | 10.00 | A | O |
| ATOM | 1424 | N | ASP | A | 165 | 9.715 | −13.088 | −9.352 | 1.00 | 10.00 | A | N |
| ATOM | 1426 | CA | ASP | A | 165 | 10.639 | −13.617 | −10.345 | 1.00 | 10.00 | A | C |
| ATOM | 1427 | CB | ASP | A | 165 | 10.725 | −12.644 | −11.523 | 1.00 | 10.00 | A | C |
| ATOM | 1428 | CG | ASP | A | 165 | 11.319 | −13.267 | −12.766 | 1.00 | 10.00 | A | C |
| ATOM | 1429 | OD1 | ASP | A | 165 | 12.366 | −12.773 | −13.241 | 1.00 | 10.00 | A | O |
| ATOM | 1430 | OD2 | ASP | A | 165 | 10.744 | −14.252 | −13.275 | 1.00 | 10.00 | A | O |
| ATOM | 1431 | C | ASP | A | 165 | 12.018 | −13.815 | −9.713 | 1.00 | 10.00 | A | C |
| ATOM | 1432 | O | ASP | A | 165 | 13.033 | −13.951 | −10.401 | 1.00 | 10.00 | A | O |
| ATOM | 1433 | N | ALA | A | 166 | 12.038 | −13.842 | −8.386 | 1.00 | 10.00 | A | N |
| ATOM | 1435 | CA | ALA | A | 166 | 13.270 | −14.005 | −7.629 | 1.00 | 10.00 | A | C |
| ATOM | 1436 | CB | ALA | A | 166 | 13.222 | −13.104 | −6.403 | 1.00 | 10.00 | A | C |
| ATOM | 1437 | C | ALA | A | 166 | 13.450 | −15.463 | −7.208 | 1.00 | 10.00 | A | C |
| ATOM | 1438 | O | ALA | A | 166 | 12.556 | −16.279 | −7.432 | 1.00 | 10.00 | A | O |
| ATOM | 1439 | N | PRO | A | 167 | 14.619 | −15.824 | −6.622 | 1.00 | 10.00 | A | N |
| ATOM | 1440 | CA | PRO | A | 167 | 14.886 | −17.195 | −6.157 | 1.00 | 10.00 | A | C |
| ATOM | 1441 | CB | PRO | A | 167 | 16.124 | −17.024 | −5.279 | 1.00 | 10.00 | A | C |
| ATOM | 1442 | CG | PRO | A | 167 | 16.845 | −15.866 | −5.872 | 1.00 | 10.00 | A | C |
| ATOM | 1443 | CD | PRO | A | 167 | 15.787 | −14.939 | −6.401 | 1.00 | 10.00 | A | C |
| ATOM | 1444 | C | PRO | A | 167 | 13.725 | −17.756 | −5.335 | 1.00 | 10.00 | A | C |
| ATOM | 1445 | O | PRO | A | 167 | 13.286 | −17.139 | −4.361 | 1.00 | 10.00 | A | O |
| ATOM | 1446 | N | GLU | A | 168 | 13.245 | −18.925 | −5.744 | 1.00 | 10.00 | A | N |
| ATOM | 1448 | CA | GLU | A | 168 | 12.114 | −19.591 | −5.101 | 1.00 | 10.00 | A | C |
| ATOM | 1449 | CB | GLU | A | 168 | 11.866 | −20.975 | −5.713 | 1.00 | 10.00 | A | C |
| ATOM | 1450 | CG | GLU | A | 168 | 12.156 | −21.078 | −7.201 | 1.00 | 10.00 | A | C |
| ATOM | 1451 | CD | GLU | A | 168 | 11.300 | −20.151 | −8.034 | 1.00 | 10.00 | A | C |
| ATOM | 1452 | OE1 | GLU | A | 168 | 10.088 | −20.414 | −8.164 | 1.00 | 10.00 | A | O |
| ATOM | 1453 | OE2 | GLU | A | 168 | 11.842 | −19.160 | −8.573 | 1.00 | 10.00 | A | O |
| ATOM | 1454 | C | GLU | A | 168 | 12.286 | −19.723 | −3.591 | 1.00 | 10.00 | A | C |
| ATOM | 1455 | O | GLU | A | 168 | 11.385 | −19.382 | −2.831 | 1.00 | 10.00 | A | O |
| ATOM | 1456 | N | ASP | A | 169 | 13.446 | −20.198 | −3.158 | 1.00 | 10.00 | A | N |
| ATOM | 1458 | CA | ASP | A | 169 | 13.705 | −20.386 | −1.730 | 1.00 | 10.00 | A | C |
| ATOM | 1459 | CB | ASP | A | 169 | 15.015 | −21.144 | −1.496 | 1.00 | 10.00 | A | C |
| ATOM | 1460 | CG | ASP | A | 169 | 14.800 | −22.631 | −1.321 | 1.00 | 10.00 | A | C |
| ATOM | 1461 | OD1 | ASP | A | 169 | 14.311 | −23.043 | −0.250 | 1.00 | 10.00 | A | O |
| ATOM | 1462 | OD2 | ASP | A | 169 | 15.113 | −23.399 | −2.257 | 1.00 | 10.00 | A | O |
| ATOM | 1463 | C | ASP | A | 169 | 13.710 | −19.066 | −0.968 | 1.00 | 10.00 | A | C |
| ATOM | 1464 | O | ASP | A | 169 | 13.066 | −18.936 | 0.076 | 1.00 | 10.00 | A | O |
| ATOM | 1465 | N | GLU | A | 170 | 14.428 | −18.089 | −1.501 | 1.00 | 10.00 | A | N |
| ATOM | 1467 | CA | GLU | A | 170 | 14.529 | −16.773 | −0.876 | 1.00 | 10.00 | A | C |
| ATOM | 1468 | CB | GLU | A | 170 | 15.595 | −15.913 | −1.566 | 1.00 | 10.00 | A | C |
| ATOM | 1469 | CG | GLU | A | 170 | 17.027 | −16.233 | −1.154 | 1.00 | 10.00 | A | C |
| ATOM | 1470 | CD | GLU | A | 170 | 17.441 | −17.658 | −1.480 | 1.00 | 10.00 | A | C |
| ATOM | 1471 | OE1 | GLU | A | 170 | 17.062 | −18.165 | −2.556 | 1.00 | 10.00 | A | O |
| ATOM | 1472 | OE2 | GLU | A | 170 | 18.152 | −18.282 | −0.663 | 1.00 | 10.00 | A | O |
| ATOM | 1473 | C | GLU | A | 170 | 13.178 | −16.056 | −0.840 | 1.00 | 10.00 | A | C |
| ATOM | 1474 | O | GLU | A | 170 | 12.954 | −15.192 | 0.011 | 1.00 | 10.00 | A | O |
| ATOM | 1475 | N | ARG | A | 171 | 12.276 | −16.431 | −1.754 | 1.00 | 10.00 | A | N |
| ATOM | 1477 | CA | ARG | A | 171 | 10.941 | −15.833 | −1.815 | 1.00 | 10.00 | A | C |
| ATOM | 1478 | CB | ARG | A | 171 | 10.069 | −16.517 | −2.874 | 1.00 | 10.00 | A | C |
| ATOM | 1479 | CG | ARG | A | 171 | 10.429 | −16.231 | −4.320 | 1.00 | 10.00 | A | C |
| ATOM | 1480 | CD | ARG | A | 171 | 9.415 | −16.871 | −5.261 | 1.00 | 10.00 | A | C |
| ATOM | 1481 | NE | ARG | A | 171 | 9.970 | −17.149 | −6.586 | 1.00 | 10.00 | A | N |
| ATOM | 1483 | CZ | ARG | A | 171 | 9.350 | −16.868 | −7.730 | 1.00 | 10.00 | A | C |
| ATOM | 1484 | NH1 | ARG | A | 171 | 8.158 | −16.284 | −7.722 | 1.00 | 10.00 | A | N |
| ATOM | 1487 | NH2 | ARG | A | 171 | 9.913 | −17.202 | −8.882 | 1.00 | 10.00 | A | N |
| ATOM | 1490 | C | ARG | A | 171 | 10.230 | −15.954 | −0.472 | 1.00 | 10.00 | A | C |
| ATOM | 1491 | O | ARG | A | 171 | 9.692 | −14.978 | 0.047 | 1.00 | 10.00 | A | O |
| ATOM | 1492 | N | VAL | A | 172 | 10.252 | −17.158 | 0.092 | 1.00 | 10.00 | A | N |
| ATOM | 1494 | CA | VAL | A | 172 | 9.585 | −17.432 | 1.364 | 1.00 | 10.00 | A | C |
| ATOM | 1495 | CB | VAL | A | 172 | 9.661 | −18.921 | 1.754 | 1.00 | 10.00 | A | C |
| ATOM | 1496 | CG1 | VAL | A | 172 | 8.655 | −19.232 | 2.853 | 1.00 | 10.00 | A | C |
| ATOM | 1497 | CG2 | VAL | A | 172 | 9.433 | −19.811 | 0.542 | 1.00 | 10.00 | A | C |
| ATOM | 1498 | C | VAL | A | 172 | 10.132 | −16.586 | 2.512 | 1.00 | 10.00 | A | C |
| ATOM | 1499 | O | VAL | A | 172 | 9.364 | −15.986 | 3.265 | 1.00 | 10.00 | A | O |
| ATOM | 1500 | N | LEU | A | 173 | 11.456 | −16.533 | 2.636 | 1.00 | 10.00 | A | N |
| ATOM | 1502 | CA | LEU | A | 173 | 12.084 | −15.775 | 3.714 | 1.00 | 10.00 | A | C |
| ATOM | 1503 | CB | LEU | A | 173 | 13.601 | −15.987 | 3.741 | 1.00 | 10.00 | A | C |
| ATOM | 1504 | CG | LEU | A | 173 | 14.284 | −15.757 | 5.095 | 1.00 | 10.00 | A | C |
| ATOM | 1505 | CD1 | LEU | A | 173 | 13.668 | −16.650 | 6.160 | 1.00 | 10.00 | A | C |
| ATOM | 1506 | CD2 | LEU | A | 173 | 15.783 | −15.997 | 4.999 | 1.00 | 10.00 | A | C |
| ATOM | 1507 | C | LEU | A | 173 | 11.732 | −14.290 | 3.645 | 1.00 | 10.00 | A | C |
| ATOM | 1508 | O | LEU | A | 173 | 11.434 | −13.667 | 4.666 | 1.00 | 10.00 | A | O |
| ATOM | 1509 | N | ALA | A | 174 | 11.745 | −13.736 | 2.438 | 1.00 | 10.00 | A | N |
| ATOM | 1511 | CA | ALA | A | 174 | 11.415 | −12.330 | 2.242 | 1.00 | 10.00 | A | C |
| ATOM | 1512 | CB | ALA | A | 174 | 11.802 | −11.889 | 0.838 | 1.00 | 10.00 | A | C |
| ATOM | 1513 | C | ALA | A | 174 | 9.927 | −12.094 | 2.476 | 1.00 | 10.00 | A | C |
| ATOM | 1514 | O | ALA | A | 174 | 9.533 | −11.159 | 3.174 | 1.00 | 10.00 | A | O |
| ATOM | 1515 | N | ALA | A | 175 | 9.105 | −12.968 | 1.904 | 1.00 | 10.00 | A | N |
| ATOM | 1517 | CA | ALA | A | 175 | 7.657 | −12.866 | 2.036 | 1.00 | 10.00 | A | C |
| ATOM | 1518 | CB | ALA | A | 175 | 6.968 | −13.953 | 1.225 | 1.00 | 10.00 | A | C |
| ATOM | 1519 | C | ALA | A | 175 | 7.215 | −12.928 | 3.495 | 1.00 | 10.00 | A | C |
| ATOM | 1520 | O | ALA | A | 175 | 6.377 | −12.137 | 3.926 | 1.00 | 10.00 | A | O |
| ATOM | 1521 | N | LYS | A | 176 | 7.794 | −13.857 | 4.252 | 1.00 | 10.00 | A | N |
| ATOM | 1523 | CA | LYS | A | 176 | 7.446 | −14.021 | 5.657 | 1.00 | 10.00 | A | C |
| ATOM | 1524 | CB | LYS | A | 176 | 8.114 | −15.268 | 6.238 | 1.00 | 10.00 | A | C |
| ATOM | 1525 | CG | LYS | A | 176 | 7.570 | −15.678 | 7.596 | 1.00 | 10.00 | A | C |
| ATOM | 1526 | CD | LYS | A | 176 | 8.396 | −16.797 | 8.206 | 1.00 | 10.00 | A | C |
| ATOM | 1527 | CE | LYS | A | 176 | 7.868 | −17.183 | 9.577 | 1.00 | 10.00 | A | C |
| ATOM | 1528 | NZ | LYS | A | 176 | 8.844 | −18.002 | 10.334 | 1.00 | 10.00 | A | N |
| ATOM | 1532 | C | LYS | A | 176 | 7.834 | −12.787 | 6.465 | 1.00 | 10.00 | A | C |
| ATOM | 1533 | O | LYS | A | 176 | 7.104 | −12.366 | 7.361 | 1.00 | 10.00 | A | O |
| ATOM | 1534 | N | GLU | A | 177 | 8.975 | −12.204 | 6.126 | 1.00 | 10.00 | A | N |
| ATOM | 1536 | CA | GLU | A | 177 | 9.464 | −11.015 | 6.805 | 1.00 | 10.00 | A | C |
| ATOM | 1537 | CB | GLU | A | 177 | 10.814 | −10.607 | 6.213 | 1.00 | 10.00 | A | C |
| ATOM | 1538 | CG | GLU | A | 177 | 11.203 | −9.162 | 6.475 | 1.00 | 10.00 | A | C |
| ATOM | 1539 | CD | GLU | A | 177 | 11.790 | −8.490 | 5.250 | 1.00 | 10.00 | A | C |
| ATOM | 1540 | OE1 | GLU | A | 177 | 11.029 | −7.842 | 4.501 | 1.00 | 10.00 | A | O |
| ATOM | 1541 | OE2 | GLU | A | 177 | 13.014 | −8.600 | 5.032 | 1.00 | 10.00 | A | O |
| ATOM | 1542 | C | GLU | A | 177 | 8.469 | −9.871 | 6.643 | 1.00 | 10.00 | A | C |
| ATOM | 1543 | O | GLU | A | 177 | 8.083 | −9.211 | 7.614 | 1.00 | 10.00 | A | O |
| ATOM | 1544 | N | CYS | A | 178 | 8.045 | −9.665 | 5.409 | 1.00 | 10.00 | A | N |
| ATOM | 1546 | CA | CYS | A | 178 | 7.114 | −8.602 | 5.090 | 1.00 | 10.00 | A | C |
| ATOM | 1547 | CB | CYS | A | 178 | 7.025 | −8.399 | 3.581 | 1.00 | 10.00 | A | C |
| ATOM | 1548 | SG | CYS | A | 178 | 8.560 | −7.811 | 2.818 | 1.00 | 10.00 | A | S |
| ATOM | 1550 | C | CYS | A | 178 | 5.728 | −8.816 | 5.698 | 1.00 | 10.00 | A | C |
| ATOM | 1551 | O | CYS | A | 178 | 4.987 | −7.860 | 5.893 | 1.00 | 10.00 | A | O |
| ATOM | 1552 | N | THR | A | 179 | 5.376 | −10.056 | 6.003 | 1.00 | 10.00 | A | N |
| ATOM | 1554 | CA | THR | A | 179 | 4.070 | −10.333 | 6.583 | 1.00 | 10.00 | A | C |
| ATOM | 1555 | CB | THR | A | 179 | 3.484 | −11.672 | 6.103 | 1.00 | 10.00 | A | C |
| ATOM | 1556 | OG1 | THR | A | 179 | 4.540 | −12.559 | 5.720 | 1.00 | 10.00 | A | O |
| ATOM | 1558 | CG2 | THR | A | 179 | 2.544 | −11.452 | 4.929 | 1.00 | 10.00 | A | C |
| ATOM | 1559 | C | THR | A | 179 | 4.092 | −10.308 | 8.107 | 1.00 | 10.00 | A | C |
| ATOM | 1560 | O | THR | A | 179 | 3.155 | −9.823 | 8.739 | 1.00 | 10.00 | A | O |
| ATOM | 1561 | N | ASN | A | 180 | 5.166 | −10.807 | 8.695 | 1.00 | 10.00 | A | N |
| ATOM | 1563 | CA | ASN | A | 180 | 5.279 | −10.863 | 10.146 | 1.00 | 10.00 | A | C |
| ATOM | 1564 | CB | ASN | A | 180 | 6.192 | −12.015 | 10.569 | 1.00 | 10.00 | A | C |
| ATOM | 1565 | CG | ASN | A | 180 | 5.905 | −12.507 | 11.974 | 1.00 | 10.00 | A | C |
| ATOM | 1566 | OD1 | ASN | A | 180 | 6.532 | −12.077 | 12.943 | 1.00 | 10.00 | A | O |
| ATOM | 1567 | ND2 | ASN | A | 180 | 4.948 | −13.416 | 12.098 | 1.00 | 10.00 | A | N |
| ATOM | 1570 | C | ASN | A | 180 | 5.765 | −9.552 | 10.757 | 1.00 | 10.00 | A | C |
| ATOM | 1571 | O | ASN | A | 180 | 5.449 | −9.248 | 11.904 | 1.00 | 10.00 | A | O |
| ATOM | 1572 | N | ILE | A | 181 | 6.523 | −8.768 | 10.001 | 1.00 | 10.00 | A | N |
| ATOM | 1574 | CA | ILE | A | 181 | 7.052 | −7.511 | 10.524 | 1.00 | 10.00 | A | C |
| ATOM | 1575 | CB | ILE | A | 181 | 8.595 | −7.507 | 10.550 | 1.00 | 10.00 | A | C |
| ATOM | 1576 | CG1 | ILE | A | 181 | 9.119 | −8.725 | 11.314 | 1.00 | 10.00 | A | C |
| ATOM | 1577 | CG2 | ILE | A | 181 | 9.109 | −6.225 | 11.188 | 1.00 | 10.00 | A | C |
| ATOM | 1578 | CD1 | ILE | A | 181 | 10.611 | −8.927 | 11.183 | 1.00 | 10.00 | A | C |
| ATOM | 1579 | C | ILE | A | 181 | 6.549 | −6.276 | 9.769 | 1.00 | 10.00 | A | C |
| ATOM | 1580 | O | ILE | A | 181 | 5.897 | −5.412 | 10.354 | 1.00 | 10.00 | A | O |
| ATOM | 1581 | N | TYR | A | 182 | 6.843 | −6.202 | 8.476 | 1.00 | 10.00 | A | N |
| ATOM | 1583 | CA | TYR | A | 182 | 6.452 | −5.047 | 7.659 | 1.00 | 10.00 | A | C |
| ATOM | 1584 | CB | TYR | A | 182 | 7.021 | −5.135 | 6.244 | 1.00 | 10.00 | A | C |
| ATOM | 1585 | CG | TYR | A | 182 | 8.420 | −4.581 | 6.130 | 1.00 | 10.00 | A | C |
| ATOM | 1586 | CD1 | TYR | A | 182 | 8.630 | −3.243 | 5.824 | 1.00 | 10.00 | A | C |
| ATOM | 1587 | CD2 | TYR | A | 182 | 9.532 | −5.388 | 6.333 | 1.00 | 10.00 | A | C |
| ATOM | 1588 | CE1 | TYR | A | 182 | 9.904 | −2.724 | 5.721 | 1.00 | 10.00 | A | C |
| ATOM | 1589 | CE2 | TYR | A | 182 | 10.812 | −4.876 | 6.232 | 1.00 | 10.00 | A | C |
| ATOM | 1590 | CZ | TYR | A | 182 | 10.990 | −3.543 | 5.927 | 1.00 | 10.00 | A | C |
| ATOM | 1591 | OH | TYR | A | 182 | 12.262 | −3.028 | 5.817 | 1.00 | 10.00 | A | O |
| ATOM | 1593 | C | TYR | A | 182 | 4.945 | −4.769 | 7.638 | 1.00 | 10.00 | A | C |
| ATOM | 1594 | O | TYR | A | 182 | 4.517 | −3.637 | 7.870 | 1.00 | 10.00 | A | O |
| ATOM | 1595 | N | ALA | A | 183 | 4.151 | −5.791 | 7.353 | 1.00 | 10.00 | A | N |
| ATOM | 1597 | CA | ALA | A | 183 | 2.689 | −5.648 | 7.305 | 1.00 | 10.00 | A | C |
| ATOM | 1598 | CB | ALA | A | 183 | 1.996 | −6.964 | 6.969 | 1.00 | 10.00 | A | C |
| ATOM | 1599 | C | ALA | A | 183 | 2.113 | −5.004 | 8.580 | 1.00 | 10.00 | A | C |
| ATOM | 1600 | O | ALA | A | 183 | 1.451 | −3.964 | 8.500 | 1.00 | 10.00 | A | O |
| ATOM | 1601 | N | PRO | A | 184 | 2.352 | −5.590 | 9.777 | 1.00 | 10.00 | A | N |
| ATOM | 1602 | CA | PRO | A | 184 | 1.858 | −5.015 | 11.034 | 1.00 | 10.00 | A | C |
| ATOM | 1603 | CB | PRO | A | 184 | 2.430 | −5.941 | 12.111 | 1.00 | 10.00 | A | C |
| ATOM | 1604 | CG | PRO | A | 184 | 2.692 | −7.224 | 11.403 | 1.00 | 10.00 | A | C |
| ATOM | 1605 | CD | PRO | A | 184 | 3.086 | −6.847 | 10.007 | 1.00 | 10.00 | A | C |
| ATOM | 1606 | C | PRO | A | 184 | 2.379 | −3.593 | 11.240 | 1.00 | 10.00 | A | C |
| ATOM | 1607 | O | PRO | A | 184 | 1.650 | −2.719 | 11.708 | 1.00 | 10.00 | A | O |
| ATOM | 1608 | N | LEU | A | 185 | 3.638 | −3.372 | 10.864 | 1.00 | 10.00 | A | N |
| ATOM | 1610 | CA | LEU | A | 185 | 4.268 | −2.067 | 10.996 | 1.00 | 10.00 | A | C |
| ATOM | 1611 | CB | LEU | A | 185 | 5.729 | −2.141 | 10.550 | 1.00 | 10.00 | A | C |
| ATOM | 1612 | CG | LEU | A | 185 | 6.544 | −0.853 | 10.685 | 1.00 | 10.00 | A | C |
| ATOM | 1613 | CD1 | LEU | A | 185 | 7.161 | −0.745 | 12.072 | 1.00 | 10.00 | A | C |
| ATOM | 1614 | CD2 | LEU | A | 185 | 7.615 | −0.778 | 9.609 | 1.00 | 10.00 | A | C |
| ATOM | 1615 | C | LEU | A | 185 | 3.528 | −1.020 | 10.169 | 1.00 | 10.00 | A | C |
| ATOM | 1616 | O | LEU | A | 185 | 3.193 | 0.054 | 10.668 | 1.00 | 10.00 | A | O |
| ATOM | 1617 | N | ALA | A | 186 | 3.268 | −1.350 | 8.907 | 1.00 | 10.00 | A | N |
| ATOM | 1619 | CA | ALA | A | 186 | 2.562 | −0.452 | 8.000 | 1.00 | 10.00 | A | C |
| ATOM | 1620 | CB | ALA | A | 186 | 2.568 | −1.020 | 6.589 | 1.00 | 10.00 | A | C |
| ATOM | 1621 | C | ALA | A | 186 | 1.133 | −0.227 | 8.473 | 1.00 | 10.00 | A | C |
| ATOM | 1622 | O | ALA | A | 186 | 0.579 | 0.869 | 8.336 | 1.00 | 10.00 | A | O |
| ATOM | 1623 | N | ASN | A | 187 | 0.545 | −1.275 | 9.039 | 1.00 | 10.00 | A | N |
| ATOM | 1625 | CA | ASN | A | 187 | −0.816 | −1.206 | 9.552 | 1.00 | 10.00 | A | C |
| ATOM | 1626 | CB | ASN | A | 187 | −1.311 | −2.587 | 10.002 | 1.00 | 10.00 | A | C |
| ATOM | 1627 | CG | ASN | A | 187 | −2.791 | −2.592 | 10.344 | 1.00 | 10.00 | A | C |
| ATOM | 1628 | OD1 | ASN | A | 187 | −3.646 | −2.518 | 9.459 | 1.00 | 10.00 | A | O |
| ATOM | 1629 | ND2 | ASN | A | 187 | −3.110 | −2.694 | 11.624 | 1.00 | 10.00 | A | N |
| ATOM | 1632 | C | ASN | A | 187 | −0.888 | −0.224 | 10.711 | 1.00 | 10.00 | A | C |
| ATOM | 1633 | O | ASN | A | 187 | −1.810 | 0.586 | 10.794 | 1.00 | 10.00 | A | O |
| ATOM | 1634 | N | ARG | A | 188 | 0.103 | −0.286 | 11.589 | 1.00 | 10.00 | A | N |
| ATOM | 1636 | CA | ARG | A | 188 | 0.161 | 0.599 | 12.744 | 1.00 | 10.00 | A | C |
| ATOM | 1637 | CB | ARG | A | 188 | 1.266 | 0.162 | 13.707 | 1.00 | 10.00 | A | C |
| ATOM | 1638 | CG | ARG | A | 188 | 1.057 | −1.213 | 14.322 | 1.00 | 10.00 | A | C |
| ATOM | 1639 | CD | ARG | A | 188 | 2.311 | −1.693 | 15.035 | 1.00 | 10.00 | A | C |
| ATOM | 1640 | NE | ARG | A | 188 | 2.301 | −3.136 | 15.251 | 1.00 | 10.00 | A | N |
| ATOM | 1642 | CZ | ARG | A | 188 | 3.353 | −3.837 | 15.680 | 1.00 | 10.00 | A | C |
| ATOM | 1643 | NH1 | ARG | A | 188 | 4.508 | −3.234 | 15.941 | 1.00 | 10.00 | A | N |
| ATOM | 1646 | NH2 | ARG | A | 188 | 3.253 | −5.150 | 15.838 | 1.00 | 10.00 | A | N |
| ATOM | 1649 | C | ARG | A | 188 | 0.392 | 2.042 | 12.311 | 1.00 | 10.00 | A | C |
| ATOM | 1650 | O | ARG | A | 188 | −0.029 | 2.976 | 12.985 | 1.00 | 10.00 | A | O |
| ATOM | 1651 | N | LEU | A | 189 | 1.061 | 2.214 | 11.177 | 1.00 | 10.00 | A | N |
| ATOM | 1653 | CA | LEU | A | 189 | 1.341 | 3.540 | 10.650 | 1.00 | 10.00 | A | C |
| ATOM | 1654 | CB | LEU | A | 189 | 2.507 | 3.491 | 9.662 | 1.00 | 10.00 | A | C |
| ATOM | 1655 | CG | LEU | A | 189 | 3.177 | 4.828 | 9.334 | 1.00 | 10.00 | A | C |
| ATOM | 1656 | CD1 | LEU | A | 189 | 3.655 | 5.515 | 10.604 | 1.00 | 10.00 | A | C |
| ATOM | 1657 | CD2 | LEU | A | 189 | 4.339 | 4.614 | 8.378 | 1.00 | 10.00 | A | C |
| ATOM | 1658 | C | LEU | A | 189 | 0.102 | 4.132 | 9.987 | 1.00 | 10.00 | A | C |
| ATOM | 1659 | O | LEU | A | 189 | −0.094 | 5.347 | 9.998 | 1.00 | 10.00 | A | O |
| ATOM | 1660 | N | GLY | A | 190 | −0.728 | 3.268 | 9.417 | 1.00 | 10.00 | A | N |
| ATOM | 1662 | CA | GLY | A | 190 | −1.946 | 3.721 | 8.774 | 1.00 | 10.00 | A | C |
| ATOM | 1663 | C | GLY | A | 190 | −1.930 | 3.535 | 7.270 | 1.00 | 10.00 | A | C |
| ATOM | 1664 | O | GLY | A | 190 | −2.860 | 3.951 | 6.577 | 1.00 | 10.00 | A | O |
| ATOM | 1665 | N | ILE | A | 191 | −0.887 | 2.902 | 6.757 | 1.00 | 10.00 | A | N |
| ATOM | 1667 | CA | ILE | A | 191 | −0.776 | 2.677 | 5.322 | 1.00 | 10.00 | A | C |
| ATOM | 1668 | CB | ILE | A | 191 | 0.684 | 2.750 | 4.831 | 1.00 | 10.00 | A | C |
| ATOM | 1669 | CG1 | ILE | A | 191 | 1.391 | 3.960 | 5.441 | 1.00 | 10.00 | A | C |
| ATOM | 1670 | CG2 | ILE | A | 191 | 0.720 | 2.835 | 3.312 | 1.00 | 10.00 | A | C |
| ATOM | 1671 | CD1 | ILE | A | 191 | 2.852 | 4.066 | 5.070 | 1.00 | 10.00 | A | C |
| ATOM | 1672 | C | ILE | A | 191 | −1.380 | 1.336 | 4.922 | 1.00 | 10.00 | A | C |
| ATOM | 1673 | O | ILE | A | 191 | −0.690 | 0.316 | 4.876 | 1.00 | 10.00 | A | O |
| ATOM | 1674 | N | GLY | A | 192 | −2.676 | 1.343 | 4.640 | 1.00 | 10.00 | A | N |
| ATOM | 1676 | CA | GLY | A | 192 | −3.354 | 0.131 | 4.238 | 1.00 | 10.00 | A | C |
| ATOM | 1677 | C | GLY | A | 192 | −3.157 | −0.165 | 2.767 | 1.00 | 10.00 | A | C |
| ATOM | 1678 | O | GLY | A | 192 | −3.158 | −1.325 | 2.356 | 1.00 | 10.00 | A | O |
| ATOM | 1679 | N | GLN | A | 193 | −2.972 | 0.891 | 1.982 | 1.00 | 10.00 | A | N |
| ATOM | 1681 | CA | GLN | A | 193 | −2.771 | 0.773 | 0.544 | 1.00 | 10.00 | A | C |
| ATOM | 1682 | CB | GLN | A | 193 | −2.638 | 2.161 | −0.085 | 1.00 | 10.00 | A | C |
| ATOM | 1683 | CG | GLN | A | 193 | −2.041 | 2.171 | −1.483 | 1.00 | 10.00 | A | C |
| ATOM | 1684 | CD | GLN | A | 193 | −2.050 | 3.551 | −2.103 | 1.00 | 10.00 | A | C |
| ATOM | 1685 | OE1 | GLN | A | 193 | −1.111 | 4.330 | −1.931 | 1.00 | 10.00 | A | O |
| ATOM | 1686 | NE2 | GLN | A | 193 | −3.108 | 3.861 | −2.832 | 1.00 | 10.00 | A | N |
| ATOM | 1689 | C | GLN | A | 193 | −1.553 | −0.083 | 0.222 | 1.00 | 10.00 | A | C |
| ATOM | 1690 | O | GLN | A | 193 | −1.592 | −0.916 | −0.679 | 1.00 | 10.00 | A | O |
| ATOM | 1691 | N | LEU | A | 194 | −0.473 | 0.115 | 0.961 | 1.00 | 10.00 | A | N |
| ATOM | 1693 | CA | LEU | A | 194 | 0.737 | −0.660 | 0.728 | 1.00 | 10.00 | A | C |
| ATOM | 1694 | CB | LEU | A | 194 | 1.975 | 0.096 | 1.212 | 1.00 | 10.00 | A | C |
| ATOM | 1695 | CG | LEU | A | 194 | 2.274 | 1.418 | 0.500 | 1.00 | 10.00 | A | C |
| ATOM | 1696 | CD1 | LEU | A | 194 | 3.509 | 2.087 | 1.089 | 1.00 | 10.00 | A | C |
| ATOM | 1697 | CD2 | LEU | A | 194 | 2.436 | 1.198 | −0.997 | 1.00 | 10.00 | A | C |
| ATOM | 1698 | C | LEU | A | 194 | 0.645 | −2.020 | 1.399 | 1.00 | 10.00 | A | C |
| ATOM | 1699 | O | LEU | A | 194 | 1.001 | −3.036 | 0.810 | 1.00 | 10.00 | A | O |
| ATOM | 1700 | N | LYS | A | 195 | 0.121 | −2.030 | 2.617 | 1.00 | 10.00 | A | N |
| ATOM | 1702 | CA | LYS | A | 195 | −0.008 | −3.255 | 3.397 | 1.00 | 10.00 | A | C |
| ATOM | 1703 | CB | LYS | A | 195 | −0.677 | −2.990 | 4.748 | 1.00 | 10.00 | A | C |
| ATOM | 1704 | CG | LYS | A | 195 | −0.689 | −4.203 | 5.668 | 1.00 | 10.00 | A | C |
| ATOM | 1705 | CD | LYS | A | 195 | −1.524 | −3.958 | 6.916 | 1.00 | 10.00 | A | C |
| ATOM | 1706 | CE | LYS | A | 195 | −3.002 | −3.809 | 6.590 | 1.00 | 10.00 | A | C |
| ATOM | 1707 | NZ | LYS | A | 195 | −3.704 | −5.117 | 6.481 | 1.00 | 10.00 | A | N |
| ATOM | 1711 | C | LYS | A | 195 | −0.733 | −4.371 | 2.648 | 1.00 | 10.00 | A | C |
| ATOM | 1712 | O | LYS | A | 195 | −0.215 | −5.482 | 2.547 | 1.00 | 10.00 | A | O |
| ATOM | 1713 | N | TRP | A | 196 | −1.912 | −4.075 | 2.100 | 1.00 | 10.00 | A | N |
| ATOM | 1715 | CA | TRP | A | 196 | −2.679 | −5.093 | 1.379 | 1.00 | 10.00 | A | C |
| ATOM | 1716 | CB | TRP | A | 196 | −4.093 | −4.609 | 1.000 | 1.00 | 10.00 | A | C |
| ATOM | 1717 | CG | TRP | A | 196 | −4.189 | −3.847 | −0.293 | 1.00 | 10.00 | A | C |
| ATOM | 1718 | CD1 | TRP | A | 196 | −4.110 | −2.494 | −0.460 | 1.00 | 10.00 | A | C |
| ATOM | 1719 | CD2 | TRP | A | 196 | −4.398 | −4.397 | −1.601 | 1.00 | 10.00 | A | C |
| ATOM | 1720 | NE1 | TRP | A | 196 | −4.245 | −2.170 | −1.787 | 1.00 | 10.00 | A | N |
| ATOM | 1722 | CE2 | TRP | A | 196 | −4.421 | −3.321 | −2.508 | 1.00 | 10.00 | A | C |
| ATOM | 1723 | CE3 | TRP | A | 196 | −4.559 | −5.695 | −2.093 | 1.00 | 10.00 | A | C |
| ATOM | 1724 | CZ2 | TRP | A | 196 | −4.601 | −3.504 | −3.878 | 1.00 | 10.00 | A | C |
| ATOM | 1725 | CZ3 | TRP | A | 196 | −4.738 | −5.872 | −3.451 | 1.00 | 10.00 | A | C |
| ATOM | 1726 | CH2 | TRP | A | 196 | −4.757 | −4.784 | −4.329 | 1.00 | 10.00 | A | C |
| ATOM | 1727 | C | TRP | A | 196 | −1.903 | −5.643 | 0.180 | 1.00 | 10.00 | A | C |
| ATOM | 1728 | O | TRP | A | 196 | −1.952 | −6.838 | −0.107 | 1.00 | 10.00 | A | O |
| ATOM | 1729 | N | GLU | A | 197 | −1.162 | −4.771 | −0.495 | 1.00 | 10.00 | A | N |
| ATOM | 1731 | CA | GLU | A | 197 | −0.370 | −5.178 | −1.648 | 1.00 | 10.00 | A | C |
| ATOM | 1732 | CB | GLU | A | 197 | 0.177 | −3.952 | −2.385 | 1.00 | 10.00 | A | C |
| ATOM | 1733 | CG | GLU | A | 197 | 0.348 | −4.158 | −3.885 | 1.00 | 10.00 | A | C |
| ATOM | 1734 | CD | GLU | A | 197 | 0.817 | −2.910 | −4.604 | 1.00 | 10.00 | A | C |
| ATOM | 1735 | OE1 | GLU | A | 197 | −0.039 | −2.087 | −4.991 | 1.00 | 10.00 | A | O |
| ATOM | 1736 | OE2 | GLU | A | 197 | 2.041 | −2.745 | −4.790 | 1.00 | 10.00 | A | O |
| ATOM | 1737 | C | GLU | A | 197 | 0.773 | −6.086 | −1.199 | 1.00 | 10.00 | A | C |
| ATOM | 1738 | O | GLU | A | 197 | 1.124 | −7.049 | −1.874 | 1.00 | 10.00 | A | O |
| ATOM | 1739 | N | LEU | A | 198 | 1.341 | −5.767 | −0.044 | 1.00 | 10.00 | A | N |
| ATOM | 1741 | CA | LEU | A | 198 | 2.438 | −6.548 | 0.516 | 1.00 | 10.00 | A | C |
| ATOM | 1742 | CB | LEU | A | 198 | 3.055 | −5.839 | 1.727 | 1.00 | 10.00 | A | C |
| ATOM | 1743 | CG | LEU | A | 198 | 3.513 | −4.392 | 1.537 | 1.00 | 10.00 | A | C |
| ATOM | 1744 | CD1 | LEU | A | 198 | 4.052 | −3.833 | 2.845 | 1.00 | 10.00 | A | C |
| ATOM | 1745 | CD2 | LEU | A | 198 | 4.546 | −4.277 | 0.425 | 1.00 | 10.00 | A | C |
| ATOM | 1746 | C | LEU | A | 198 | 1.961 | −7.929 | 0.940 | 1.00 | 10.00 | A | C |
| ATOM | 1747 | O | LEU | A | 198 | 2.521 | −8.939 | 0.520 | 1.00 | 10.00 | A | O |
| ATOM | 1748 | N | GLU | A | 199 | 0.917 | −7.954 | 1.768 | 1.00 | 10.00 | A | N |
| ATOM | 1750 | CA | GLU | A | 199 | 0.349 | −9.197 | 2.283 | 1.00 | 10.00 | A | C |
| ATOM | 1751 | CB | GLU | A | 199 | −0.916 | −8.919 | 3.114 | 1.00 | 10.00 | A | C |
| ATOM | 1752 | CG | GLU | A | 199 | −0.699 | −8.019 | 4.327 | 1.00 | 10.00 | A | C |
| ATOM | 1753 | CD | GLU | A | 199 | −1.978 | −7.738 | 5.100 | 1.00 | 10.00 | A | C |
| ATOM | 1754 | OE1 | GLU | A | 199 | −2.241 | −8.438 | 6.100 | 1.00 | 10.00 | A | O |
| ATOM | 1755 | OE2 | GLU | A | 199 | −2.716 | −6.800 | 4.726 | 1.00 | 10.00 | A | O |
| ATOM | 1756 | C | GLU | A | 199 | 0.045 | −10.188 | 1.159 | 1.00 | 10.00 | A | C |
| ATOM | 1757 | O | GLU | A | 199 | 0.469 | −11.347 | 1.210 | 1.00 | 10.00 | A | O |
| ATOM | 1758 | N | ASP | A | 200 | −0.663 | −9.718 | 0.134 | 1.00 | 10.00 | A | N |
| ATOM | 1760 | CA | ASP | A | 200 | −1.027 | −10.562 | −1.003 | 1.00 | 10.00 | A | C |
| ATOM | 1761 | CB | ASP | A | 200 | −1.918 | −9.805 | −1.988 | 1.00 | 10.00 | A | C |
| ATOM | 1762 | CG | ASP | A | 200 | −2.257 | −10.643 | −3.203 | 1.00 | 10.00 | A | C |
| ATOM | 1763 | OD1 | ASP | A | 200 | −1.654 | −10.417 | −4.270 | 1.00 | 10.00 | A | O |
| ATOM | 1764 | OD2 | ASP | A | 200 | −3.114 | −11.547 | −3.080 | 1.00 | 10.00 | A | O |
| ATOM | 1765 | C | ASP | A | 200 | 0.210 | −11.077 | −1.729 | 1.00 | 10.00 | A | C |
| ATOM | 1766 | O | ASP | A | 200 | 0.329 | −12.275 | −2.003 | 1.00 | 10.00 | A | O |
| ATOM | 1767 | N | TYR | A | 201 | 1.146 | −10.170 | −2.008 | 1.00 | 10.00 | A | N |
| ATOM | 1769 | CA | TYR | A | 201 | 2.377 | −10.522 | −2.715 | 1.00 | 10.00 | A | C |
| ATOM | 1770 | CB | TYR | A | 201 | 3.223 | −9.283 | −3.009 | 1.00 | 10.00 | A | C |
| ATOM | 1771 | CG | TYR | A | 201 | 2.684 | −8.420 | −4.130 | 1.00 | 10.00 | A | C |
| ATOM | 1772 | CD1 | TYR | A | 201 | 1.479 | −8.724 | −4.758 | 1.00 | 10.00 | A | C |
| ATOM | 1773 | CD2 | TYR | A | 201 | 3.376 | −7.295 | −4.557 | 1.00 | 10.00 | A | C |
| ATOM | 1774 | CE1 | TYR | A | 201 | 0.984 | −7.933 | −5.774 | 1.00 | 10.00 | A | C |
| ATOM | 1775 | CE2 | TYR | A | 201 | 2.885 | −6.499 | −5.574 | 1.00 | 10.00 | A | C |
| ATOM | 1776 | CZ | TYR | A | 201 | 1.689 | −6.823 | −6.178 | 1.00 | 10.00 | A | C |
| ATOM | 1777 | OH | TYR | A | 201 | 1.196 | −6.033 | −7.192 | 1.00 | 10.00 | A | O |
| ATOM | 1779 | C | TYR | A | 201 | 3.191 | −11.558 | −1.951 | 1.00 | 10.00 | A | C |
| ATOM | 1780 | O | TYR | A | 201 | 3.926 | −12.346 | −2.547 | 1.00 | 10.00 | A | O |
| ATOM | 1781 | N | CYS | A | 202 | 3.057 | −11.557 | −0.635 | 1.00 | 10.00 | A | N |
| ATOM | 1783 | CA | CYS | A | 202 | 3.765 | −12.516 | 0.200 | 1.00 | 10.00 | A | C |
| ATOM | 1784 | CB | CYS | A | 202 | 3.782 | −12.058 | 1.657 | 1.00 | 10.00 | A | C |
| ATOM | 1785 | SG | CYS | A | 202 | 4.541 | −10.440 | 1.926 | 1.00 | 10.00 | A | S |
| ATOM | 1787 | C | CYS | A | 202 | 3.113 | −13.889 | 0.094 | 1.00 | 10.00 | A | C |
| ATOM | 1788 | O | CYS | A | 202 | 3.796 | −14.906 | −0.013 | 1.00 | 10.00 | A | O |
| ATOM | 1789 | N | PHE | A | 203 | 1.782 | −13.905 | 0.093 | 1.00 | 10.00 | A | N |
| ATOM | 1791 | CA | PHE | A | 203 | 1.028 | −15.152 | 0.009 | 1.00 | 10.00 | A | C |
| ATOM | 1792 | CB | PHE | A | 203 | −0.449 | −14.926 | 0.354 | 1.00 | 10.00 | A | C |
| ATOM | 1793 | CG | PHE | A | 203 | −0.680 | −14.527 | 1.787 | 1.00 | 10.00 | A | C |
| ATOM | 1794 | CD1 | PHE | A | 203 | −1.556 | −13.502 | 2.103 | 1.00 | 10.00 | A | C |
| ATOM | 1795 | CD2 | PHE | A | 203 | −0.016 | −15.173 | 2.816 | 1.00 | 10.00 | A | C |
| ATOM | 1796 | CE1 | PHE | A | 203 | −1.767 | −13.130 | 3.414 | 1.00 | 10.00 | A | C |
| ATOM | 1797 | CE2 | PHE | A | 203 | −0.224 | −14.807 | 4.132 | 1.00 | 10.00 | A | C |
| ATOM | 1798 | CZ | PHE | A | 203 | −1.101 | −13.782 | 4.431 | 1.00 | 10.00 | A | C |
| ATOM | 1799 | C | PHE | A | 203 | 1.168 | −15.817 | −1.355 | 1.00 | 10.00 | A | C |
| ATOM | 1800 | O | PHE | A | 203 | 0.830 | −16.992 | −1.505 | 1.00 | 10.00 | A | O |
| ATOM | 1801 | N | ARG | A | 204 | 1.668 | −15.057 | −2.338 | 1.00 | 10.00 | A | N |
| ATOM | 1803 | CA | ARG | A | 204 | 1.870 | −15.565 | −3.696 | 1.00 | 10.00 | A | C |
| ATOM | 1804 | CB | ARG | A | 204 | 2.630 | −14.544 | −4.556 | 1.00 | 10.00 | A | C |
| ATOM | 1805 | CG | ARG | A | 204 | 1.785 | −13.378 | −5.051 | 1.00 | 10.00 | A | C |
| ATOM | 1806 | CD | ARG | A | 204 | 2.369 | −12.766 | −6.320 | 1.00 | 10.00 | A | C |
| ATOM | 1807 | NE | ARG | A | 204 | 3.335 | −11.695 | −6.048 | 1.00 | 10.00 | A | N |
| ATOM | 1809 | CZ | ARG | A | 204 | 3.589 | −10.691 | −6.898 | 1.00 | 10.00 | A | C |
| ATOM | 1810 | NH1 | ARG | A | 204 | 2.961 | −10.623 | −8.062 | 1.00 | 10.00 | A | N |
| ATOM | 1813 | NH2 | ARG | A | 204 | 4.477 | −9.754 | −6.596 | 1.00 | 10.00 | A | N |
| ATOM | 1816 | C | ARG | A | 204 | 2.623 | −16.891 | −3.689 | 1.00 | 10.00 | A | C |
| ATOM | 1817 | O | ARG | A | 204 | 2.267 | −17.826 | −4.407 | 1.00 | 10.00 | A | O |
| ATOM | 1818 | N | TYR | A | 205 | 3.678 | −16.960 | −2.890 | 1.00 | 10.00 | A | N |
| ATOM | 1820 | CA | TYR | A | 205 | 4.466 | −18.176 | −2.784 | 1.00 | 10.00 | A | C |
| ATOM | 1821 | CB | TYR | A | 205 | 5.928 | −17.901 | −3.153 | 1.00 | 10.00 | A | C |
| ATOM | 1822 | CG | TYR | A | 205 | 6.736 | −19.142 | −3.452 | 1.00 | 10.00 | A | C |
| ATOM | 1823 | CD1 | TYR | A | 205 | 6.449 | −19.929 | −4.559 | 1.00 | 10.00 | A | C |
| ATOM | 1824 | CD2 | TYR | A | 205 | 7.789 | −19.521 | −2.634 | 1.00 | 10.00 | A | C |
| ATOM | 1825 | CE1 | TYR | A | 205 | 7.187 | −21.060 | −4.845 | 1.00 | 10.00 | A | C |
| ATOM | 1826 | CE2 | TYR | A | 205 | 8.532 | −20.654 | −2.909 | 1.00 | 10.00 | A | C |
| ATOM | 1827 | CZ | TYR | A | 205 | 8.228 | −21.419 | −4.015 | 1.00 | 10.00 | A | C |
| ATOM | 1828 | OH | TYR | A | 205 | 8.968 | −22.549 | −4.292 | 1.00 | 10.00 | A | O |
| ATOM | 1830 | C | TYR | A | 205 | 4.359 | −18.784 | −1.384 | 1.00 | 10.00 | A | C |
| ATOM | 1831 | O | TYR | A | 205 | 4.716 | −19.944 | −1.169 | 1.00 | 10.00 | A | O |
| ATOM | 1832 | N | LEU | A | 206 | 3.853 | −17.998 | −0.435 | 1.00 | 10.00 | A | N |
| ATOM | 1834 | CA | LEU | A | 206 | 3.699 | −18.457 | 0.940 | 1.00 | 10.00 | A | C |
| ATOM | 1835 | CB | LEU | A | 206 | 3.417 | −17.282 | 1.878 | 1.00 | 10.00 | A | C |
| ATOM | 1836 | CG | LEU | A | 206 | 4.529 | −16.919 | 2.866 | 1.00 | 10.00 | A | C |
| ATOM | 1837 | CD1 | LEU | A | 206 | 4.178 | −15.640 | 3.613 | 1.00 | 10.00 | A | C |
| ATOM | 1838 | CD2 | LEU | A | 206 | 4.768 | −18.060 | 3.844 | 1.00 | 10.00 | A | C |
| ATOM | 1839 | C | LEU | A | 206 | 2.587 | −19.491 | 1.052 | 1.00 | 10.00 | A | C |
| ATOM | 1840 | O | LEU | A | 206 | 2.757 | −20.529 | 1.696 | 1.00 | 10.00 | A | O |
| ATOM | 1841 | N | HIS | A | 207 | 1.455 | −19.215 | 0.417 | 1.00 | 10.00 | A | N |
| ATOM | 1843 | CA | HIS | A | 207 | 0.321 | −20.127 | 0.458 | 1.00 | 10.00 | A | C |
| ATOM | 1844 | CB | HIS | A | 207 | −0.758 | −19.616 | 1.417 | 1.00 | 10.00 | A | C |
| ATOM | 1845 | CG | HIS | A | 207 | −0.426 | −19.776 | 2.869 | 1.00 | 10.00 | A | C |
| ATOM | 1846 | ND1 | HIS | A | 207 | −0.730 | −20.932 | 3.553 | 1.00 | 10.00 | A | N |
| ATOM | 1847 | CD2 | HIS | A | 207 | 0.163 | −18.899 | 3.720 | 1.00 | 10.00 | A | C |
| ATOM | 1848 | CE1 | HIS | A | 207 | −0.323 | −20.736 | 4.795 | 1.00 | 10.00 | A | C |
| ATOM | 1849 | NE2 | HIS | A | 207 | 0.221 | −19.524 | 4.942 | 1.00 | 10.00 | A | N |
| ATOM | 1851 | C | HIS | A | 207 | −0.273 | −20.319 | −0.933 | 1.00 | 10.00 | A | C |
| ATOM | 1852 | O | HIS | A | 207 | −1.316 | −19.747 | −1.249 | 1.00 | 10.00 | A | O |
| ATOM | 1853 | N | PRO | A | 208 | 0.385 | −21.125 | −1.788 | 1.00 | 10.00 | A | N |
| ATOM | 1854 | CA | PRO | A | 208 | −0.090 | −21.385 | −3.153 | 1.00 | 10.00 | A | C |
| ATOM | 1855 | CB | PRO | A | 208 | 0.948 | −22.359 | −3.724 | 1.00 | 10.00 | A | C |
| ATOM | 1856 | CG | PRO | A | 208 | 2.153 | −22.181 | −2.865 | 1.00 | 10.00 | A | C |
| ATOM | 1857 | CD | PRO | A | 208 | 1.641 | −21.837 | −1.497 | 1.00 | 10.00 | A | C |
| ATOM | 1858 | C | PRO | A | 208 | −1.470 | −22.029 | −3.152 | 1.00 | 10.00 | A | C |
| ATOM | 1859 | O | PRO | A | 208 | −2.323 | −21.689 | −3.965 | 1.00 | 10.00 | A | O |
| ATOM | 1860 | N | THR | A | 209 | −1.688 | −22.936 | −2.210 | 1.00 | 10.00 | A | N |
| ATOM | 1862 | CA | THR | A | 209 | −2.957 | −23.638 | −2.090 | 1.00 | 10.00 | A | C |
| ATOM | 1863 | CB | THR | A | 209 | −2.857 | −24.733 | −1.016 | 1.00 | 10.00 | A | C |
| ATOM | 1864 | OG1 | THR | A | 209 | −1.729 | −24.455 | −0.174 | 1.00 | 10.00 | A | O |
| ATOM | 1866 | CG2 | THR | A | 209 | −2.679 | −26.098 | −1.656 | 1.00 | 10.00 | A | C |
| ATOM | 1867 | C | THR | A | 209 | −4.095 | −22.684 | −1.736 | 1.00 | 10.00 | A | C |
| ATOM | 1868 | O | THR | A | 209 | −5.221 | −22.844 | −2.205 | 1.00 | 10.00 | A | O |
| ATOM | 1869 | N | GLU | A | 210 | −3.797 | −21.697 | −0.900 | 1.00 | 10.00 | A | N |
| ATOM | 1871 | CA | GLU | A | 210 | −4.794 | −20.717 | −0.482 | 1.00 | 10.00 | A | C |
| ATOM | 1872 | CB | GLU | A | 210 | −4.377 | −20.031 | 0.820 | 1.00 | 10.00 | A | C |
| ATOM | 1873 | CG | GLU | A | 210 | −4.613 | −20.868 | 2.068 | 1.00 | 10.00 | A | C |
| ATOM | 1874 | CD | GLU | A | 210 | −6.082 | −21.109 | 2.340 | 1.00 | 10.00 | A | C |
| ATOM | 1875 | OE1 | GLU | A | 210 | −6.661 | −20.384 | 3.173 | 1.00 | 10.00 | A | O |
| ATOM | 1876 | OE2 | GLU | A | 210 | −6.669 | −22.021 | 1.720 | 1.00 | 10.00 | A | O |
| ATOM | 1877 | C | GLU | A | 210 | −5.026 | −19.686 | −1.575 | 1.00 | 10.00 | A | C |
| ATOM | 1878 | O | GLU | A | 210 | −6.157 | −19.290 | −1.835 | 1.00 | 10.00 | A | O |
| ATOM | 1879 | N | TYR | A | 211 | −3.949 | −19.261 | −2.218 | 1.00 | 10.00 | A | N |
| ATOM | 1881 | CA | TYR | A | 211 | −4.040 | −18.284 | −3.292 | 1.00 | 10.00 | A | C |
| ATOM | 1882 | CB | TYR | A | 211 | −2.643 | −17.831 | −3.718 | 1.00 | 10.00 | A | C |
| ATOM | 1883 | CG | TYR | A | 211 | −2.588 | −16.424 | −4.274 | 1.00 | 10.00 | A | C |
| ATOM | 1884 | CD1 | TYR | A | 211 | −1.403 | −15.910 | −4.782 | 1.00 | 10.00 | A | C |
| ATOM | 1885 | CD2 | TYR | A | 211 | −3.713 | −15.608 | −4.296 | 1.00 | 10.00 | A | C |
| ATOM | 1886 | CE1 | TYR | A | 211 | −1.340 | −14.632 | −5.298 | 1.00 | 10.00 | A | C |
| ATOM | 1887 | CE2 | TYR | A | 211 | −3.659 | −14.326 | −4.809 | 1.00 | 10.00 | A | C |
| ATOM | 1888 | CZ | TYR | A | 211 | −2.468 | −13.846 | −5.310 | 1.00 | 10.00 | A | C |
| ATOM | 1889 | OH | TYR | A | 211 | −2.405 | −12.579 | −5.833 | 1.00 | 10.00 | A | O |
| ATOM | 1891 | C | TYR | A | 211 | −4.792 | −18.869 | −4.483 | 1.00 | 10.00 | A | C |
| ATOM | 1892 | O | TYR | A | 211 | −5.521 | −18.166 | −5.175 | 1.00 | 10.00 | A | O |
| ATOM | 1893 | N | LYS | A | 212 | −4.625 | −20.169 | −4.706 | 1.00 | 10.00 | A | N |
| ATOM | 1895 | CA | LYS | A | 212 | −5.296 | −20.847 | −5.807 | 1.00 | 10.00 | A | C |
| ATOM | 1896 | CB | LYS | A | 212 | −4.426 | −21.968 | −6.384 | 1.00 | 10.00 | A | C |
| ATOM | 1897 | CG | LYS | A | 212 | −3.183 | −21.482 | −7.119 | 1.00 | 10.00 | A | C |
| ATOM | 1898 | CD | LYS | A | 212 | −3.535 | −20.513 | −8.238 | 1.00 | 10.00 | A | C |
| ATOM | 1899 | CE | LYS | A | 212 | −2.296 | −20.086 | −9.010 | 1.00 | 10.00 | A | C |
| ATOM | 1900 | NZ | LYS | A | 212 | −2.527 | −18.849 | −9.807 | 1.00 | 10.00 | A | N |
| ATOM | 1904 | C | LYS | A | 212 | −6.666 | −21.377 | −5.389 | 1.00 | 10.00 | A | C |
| ATOM | 1905 | O | LYS | A | 212 | −7.274 | −22.183 | −6.095 | 1.00 | 10.00 | A | O |
| ATOM | 1906 | N | ARG | A | 213 | −7.149 | −20.914 | −4.240 | 1.00 | 10.00 | A | N |
| ATOM | 1908 | CA | ARG | A | 213 | −8.461 | −21.313 | −3.739 | 1.00 | 10.00 | A | C |
| ATOM | 1909 | CB | ARG | A | 213 | −8.469 | −21.337 | −2.210 | 1.00 | 10.00 | A | C |
| ATOM | 1910 | CG | ARG | A | 213 | −8.790 | −22.695 | −1.613 | 1.00 | 10.00 | A | C |
| ATOM | 1911 | CD | ARG | A | 213 | −9.767 | −22.568 | −0.458 | 1.00 | 10.00 | A | C |
| ATOM | 1912 | NE | ARG | A | 213 | −9.207 | −21.839 | 0.681 | 1.00 | 10.00 | A | N |
| ATOM | 1914 | CZ | ARG | A | 213 | −9.938 | −21.191 | 1.582 | 1.00 | 10.00 | A | C |
| ATOM | 1915 | NH1 | ARG | A | 213 | −11.263 | −21.176 | 1.487 | 1.00 | 10.00 | A | N |
| ATOM | 1918 | NH2 | ARG | A | 213 | −9.343 | −20.562 | 2.588 | 1.00 | 10.00 | A | N |
| ATOM | 1921 | C | ARG | A | 213 | −9.509 | −20.327 | −4.246 | 1.00 | 10.00 | A | C |
| ATOM | 1922 | O | ARG | A | 213 | −10.680 | −20.376 | −3.864 | 1.00 | 10.00 | A | O |
| ATOM | 1923 | N | ILE | A | 214 | −9.053 | −19.415 | −5.088 | 1.00 | 10.00 | A | N |
| ATOM | 1925 | CA | ILE | A | 214 | −9.907 | −18.400 | −5.683 | 1.00 | 10.00 | A | C |
| ATOM | 1926 | CB | ILE | A | 214 | −9.062 | −17.172 | −6.111 | 1.00 | 10.00 | A | C |
| ATOM | 1927 | CG1 | ILE | A | 214 | −9.934 | −16.040 | −6.668 | 1.00 | 10.00 | A | C |
| ATOM | 1928 | CG2 | ILE | A | 214 | −7.980 | −17.570 | −7.105 | 1.00 | 10.00 | A | C |
| ATOM | 1929 | CD1 | ILE | A | 214 | −9.166 | −14.769 | −6.973 | 1.00 | 10.00 | A | C |
| ATOM | 1930 | C | ILE | A | 214 | −10.651 | −18.990 | −6.889 | 1.00 | 10.00 | A | C |
| ATOM | 1931 | O | ILE | A | 214 | −10.138 | −19.877 | −7.574 | 1.00 | 10.00 | A | O |
| ATOM | 1932 | N | ALA | A | 215 | −11.863 | −18.507 | −7.133 | 1.00 | 10.00 | A | N |
| ATOM | 1934 | CA | ALA | A | 215 | −12.676 | −18.992 | −8.241 | 1.00 | 10.00 | A | C |
| ATOM | 1935 | CB | ALA | A | 215 | −14.147 | −18.667 | −8.000 | 1.00 | 10.00 | A | C |
| ATOM | 1936 | C | ALA | A | 215 | −12.211 | −18.421 | −9.577 | 1.00 | 10.00 | A | C |
| ATOM | 1937 | O | ALA | A | 215 | −12.648 | −18.861 | −10.637 | 1.00 | 10.00 | A | O |
| ATOM | 1938 | N | LYS | A | 216 | −11.316 | −17.445 | −9.516 | 1.00 | 10.00 | A | N |
| ATOM | 1940 | CA | LYS | A | 216 | −10.789 | −16.807 | −10.716 | 1.00 | 10.00 | A | C |
| ATOM | 1941 | CB | LYS | A | 216 | −11.004 | −15.293 | −10.657 | 1.00 | 10.00 | A | C |
| ATOM | 1942 | CG | LYS | A | 216 | −12.420 | −14.876 | −10.301 | 1.00 | 10.00 | A | C |
| ATOM | 1943 | CD | LYS | A | 216 | −13.354 | −14.991 | −11.491 | 1.00 | 10.00 | A | C |
| ATOM | 1944 | CE | LYS | A | 216 | −14.774 | −14.620 | −11.105 | 1.00 | 10.00 | A | C |
| ATOM | 1945 | NZ | LYS | A | 216 | −15.662 | −14.536 | −12.290 | 1.00 | 10.00 | A | N |
| ATOM | 1949 | C | LYS | A | 216 | −9.306 | −17.108 | −10.877 | 1.00 | 10.00 | A | C |
| ATOM | 1950 | O | LYS | A | 216 | −8.492 | −16.725 | −10.039 | 1.00 | 10.00 | A | O |
| ATOM | 1951 | N | LEU | A | 217 | −8.954 | −17.776 | −11.963 | 1.00 | 10.00 | A | N |
| ATOM | 1953 | CA | LEU | A | 217 | −7.562 | −18.126 | −12.224 | 1.00 | 10.00 | A | C |
| ATOM | 1954 | CB | LEU | A | 217 | −7.460 | −19.424 | −13.030 | 1.00 | 10.00 | A | C |
| ATOM | 1955 | CG | LEU | A | 217 | −7.336 | −20.722 | −12.220 | 1.00 | 10.00 | A | C |
| ATOM | 1956 | CD1 | LEU | A | 217 | −6.180 | −20.639 | −11.233 | 1.00 | 10.00 | A | C |
| ATOM | 1957 | CD2 | LEU | A | 217 | −8.638 | −21.049 | −11.504 | 1.00 | 10.00 | A | C |
| ATOM | 1958 | C | LEU | A | 217 | −6.822 | −16.993 | −12.930 | 1.00 | 10.00 | A | C |
| ATOM | 1959 | O | LEU | A | 217 | −5.764 | −17.197 | −13.522 | 1.00 | 10.00 | A | O |
| ATOM | 1960 | N | LEU | A | 218 | −7.390 | −15.801 | −12.866 | 1.00 | 10.00 | A | N |
| ATOM | 1962 | CA | LEU | A | 218 | −6.793 | −14.630 | −13.480 | 1.00 | 10.00 | A | C |
| ATOM | 1963 | CB | LEU | A | 218 | −7.736 | −14.015 | −14.519 | 1.00 | 10.00 | A | C |
| ATOM | 1964 | CG | LEU | A | 218 | −8.122 | −14.900 | −15.701 | 1.00 | 10.00 | A | C |
| ATOM | 1965 | CD1 | LEU | A | 218 | −9.400 | −15.671 | −15.400 | 1.00 | 10.00 | A | C |
| ATOM | 1966 | CD2 | LEU | A | 218 | −8.285 | −14.065 | −16.959 | 1.00 | 10.00 | A | C |
| ATOM | 1967 | C | LEU | A | 218 | −6.486 | −13.601 | −12.407 | 1.00 | 10.00 | A | C |
| ATOM | 1968 | O | LEU | A | 218 | −7.370 | −13.216 | −11.647 | 1.00 | 10.00 | A | O |
| ATOM | 1969 | N | HIS | A | 219 | −5.237 | −13.171 | −12.336 | 1.00 | 10.00 | A | N |
| ATOM | 1971 | CA | HIS | A | 219 | −4.832 | −12.181 | −11.350 | 1.00 | 10.00 | A | C |
| ATOM | 1972 | CB | HIS | A | 219 | −3.419 | −12.457 | −10.819 | 1.00 | 10.00 | A | C |
| ATOM | 1973 | CG | HIS | A | 219 | −2.385 | −12.728 | −11.868 | 1.00 | 10.00 | A | C |
| ATOM | 1974 | ND1 | HIS | A | 219 | −1.761 | −11.710 | −12.554 | 1.00 | 10.00 | A | N |
| ATOM | 1975 | CD2 | HIS | A | 219 | −1.898 | −13.918 | −12.299 | 1.00 | 10.00 | A | C |
| ATOM | 1976 | CE1 | HIS | A | 219 | −0.911 | −12.299 | −13.375 | 1.00 | 10.00 | A | C |
| ATOM | 1977 | NE2 | HIS | A | 219 | −0.960 | −13.628 | −13.255 | 1.00 | 10.00 | A | N |
| ATOM | 1979 | C | HIS | A | 219 | −4.931 | −10.771 | −11.914 | 1.00 | 10.00 | A | C |
| ATOM | 1980 | O | HIS | A | 219 | −5.201 | −10.591 | −13.106 | 1.00 | 10.00 | A | O |
| ATOM | 1981 | N | GLU | A | 220 | −4.731 | −9.788 | −11.043 | 1.00 | 10.00 | A | N |
| ATOM | 1983 | CA | GLU | A | 220 | −4.778 | −8.379 | −11.421 | 1.00 | 10.00 | A | C |
| ATOM | 1984 | CB | GLU | A | 220 | −3.800 | −8.071 | −12.570 | 1.00 | 10.00 | A | C |
| ATOM | 1985 | CG | GLU | A | 220 | −3.893 | −6.663 | −13.143 | 1.00 | 10.00 | A | C |
| ATOM | 1986 | CD | GLU | A | 220 | −3.714 | −5.587 | −12.097 | 1.00 | 10.00 | A | C |
| ATOM | 1987 | OE1 | GLU | A | 220 | −2.558 | −5.335 | −11.694 | 1.00 | 10.00 | A | O |
| ATOM | 1988 | OE2 | GLU | A | 220 | −4.725 | −4.979 | −11.682 | 1.00 | 10.00 | A | O |
| ATOM | 1989 | C | GLU | A | 220 | −6.205 | −7.946 | −11.756 | 1.00 | 10.00 | A | C |
| ATOM | 1990 | O | GLU | A | 220 | −6.600 | −7.894 | −12.927 | 1.00 | 10.00 | A | O |
| ATOM | 1991 | N | ARG | A | 221 | −6.976 | −7.649 | −10.726 | 1.00 | 10.00 | A | N |
| ATOM | 1993 | CA | ARG | A | 221 | −8.346 | −7.215 | −10.908 | 1.00 | 10.00 | A | C |
| ATOM | 1994 | CB | ARG | A | 221 | −9.288 | −8.010 | −10.003 | 1.00 | 10.00 | A | C |
| ATOM | 1995 | CG | ARG | A | 221 | −10.763 | −7.784 | −10.289 | 1.00 | 10.00 | A | C |
| ATOM | 1996 | CD | ARG | A | 221 | −11.177 | −8.412 | −11.609 | 1.00 | 10.00 | A | C |
| ATOM | 1997 | NE | ARG | A | 221 | −11.257 | −9.863 | −11.514 | 1.00 | 10.00 | A | N |
| ATOM | 1999 | CZ | ARG | A | 221 | −11.376 | −10.679 | −12.552 | 1.00 | 10.00 | A | C |
| ATOM | 2000 | NH1 | ARG | A | 221 | −11.433 | −10.201 | −13.785 | 1.00 | 10.00 | A | N |
| ATOM | 2003 | NH2 | ARG | A | 221 | −11.440 | −11.981 | −12.341 | 1.00 | 10.00 | A | N |
| ATOM | 2006 | C | ARG | A | 221 | −8.455 | −5.728 | −10.614 | 1.00 | 10.00 | A | C |
| ATOM | 2007 | O | ARG | A | 221 | −9.376 | −5.060 | −11.079 | 1.00 | 10.00 | A | O |
| ATOM | 2008 | N | ARG | A | 222 | −7.501 | −5.227 | −9.831 | 1.00 | 10.00 | A | N |
| ATOM | 2010 | CA | ARG | A | 222 | −7.434 | −3.821 | −9.454 | 1.00 | 10.00 | A | C |
| ATOM | 2011 | CB | ARG | A | 222 | −6.091 | −3.510 | −8.782 | 1.00 | 10.00 | A | C |
| ATOM | 2012 | CG | ARG | A | 222 | −5.903 | −2.046 | −8.408 | 1.00 | 10.00 | A | C |
| ATOM | 2013 | CD | ARG | A | 222 | −6.302 | −1.786 | −6.966 | 1.00 | 10.00 | A | C |
| ATOM | 2014 | NE | ARG | A | 222 | −5.205 | −1.213 | −6.189 | 1.00 | 10.00 | A | N |
| ATOM | 2016 | CZ | ARG | A | 222 | −5.328 | −0.142 | −5.397 | 1.00 | 10.00 | A | C |
| ATOM | 2017 | NH1 | ARG | A | 222 | −6.500 | 0.481 | −5.280 | 1.00 | 10.00 | A | N |
| ATOM | 2020 | NH2 | ARG | A | 222 | −4.275 | 0.311 | −4.717 | 1.00 | 10.00 | A | N |
| ATOM | 2023 | C | ARG | A | 222 | −7.650 | −2.896 | −10.651 | 1.00 | 10.00 | A | C |
| ATOM | 2024 | O | ARG | A | 222 | −8.399 | −1.923 | −10.557 | 1.00 | 10.00 | A | O |
| ATOM | 2025 | N | LEU | A | 223 | −7.007 | −3.219 | −11.770 | 1.00 | 10.00 | A | N |
| ATOM | 2027 | CA | LEU | A | 223 | −7.121 | −2.427 | −12.992 | 1.00 | 10.00 | A | C |
| ATOM | 2028 | CB | LEU | A | 223 | −6.326 | −3.096 | −14.116 | 1.00 | 10.00 | A | C |
| ATOM | 2029 | CG | LEU | A | 223 | −6.186 | −2.297 | −15.412 | 1.00 | 10.00 | A | C |
| ATOM | 2030 | CD1 | LEU | A | 223 | −5.312 | −1.074 | −15.190 | 1.00 | 10.00 | A | C |
| ATOM | 2031 | CD2 | LEU | A | 223 | −5.621 | −3.171 | −16.521 | 1.00 | 10.00 | A | C |
| ATOM | 2032 | C | LEU | A | 223 | −8.580 | −2.246 | −13.422 | 1.00 | 10.00 | A | C |
| ATOM | 2033 | O | LEU | A | 223 | −8.969 | −1.194 | −13.932 | 1.00 | 10.00 | A | O |
| ATOM | 2034 | N | ASP | A | 224 | −9.387 | −3.275 | −13.215 | 1.00 | 10.00 | A | N |
| ATOM | 2036 | CA | ASP | A | 224 | −10.797 | −3.228 | −13.587 | 1.00 | 10.00 | A | C |
| ATOM | 2037 | CB | ASP | A | 224 | −11.258 | −4.611 | −14.053 | 1.00 | 10.00 | A | C |
| ATOM | 2038 | CG | ASP | A | 224 | −12.038 | −4.572 | −15.351 | 1.00 | 10.00 | A | C |
| ATOM | 2039 | OD1 | ASP | A | 224 | −11.607 | −3.865 | −16.288 | 1.00 | 10.00 | A | O |
| ATOM | 2040 | OD2 | ASP | A | 224 | −13.068 | −5.273 | −15.454 | 1.00 | 10.00 | A | O |
| ATOM | 2041 | C | ASP | A | 224 | −11.640 | −2.767 | −12.402 | 1.00 | 10.00 | A | C |
| ATOM | 2042 | O | ASP | A | 224 | −12.631 | −2.058 | −12.560 | 1.00 | 10.00 | A | O |
| ATOM | 2043 | N | ARG | A | 225 | −11.214 | −3.165 | −11.213 | 1.00 | 10.00 | A | N |
| ATOM | 2045 | CA | ARG | A | 225 | −11.898 | −2.825 | −9.976 | 1.00 | 10.00 | A | C |
| ATOM | 2046 | CB | ARG | A | 225 | −11.186 | −3.509 | −8.810 | 1.00 | 10.00 | A | C |
| ATOM | 2047 | CG | ARG | A | 225 | −12.016 | −3.657 | −7.549 | 1.00 | 10.00 | A | C |
| ATOM | 2048 | CD | ARG | A | 225 | −11.457 | −4.760 | −6.660 | 1.00 | 10.00 | A | C |
| ATOM | 2049 | NE | ARG | A | 225 | −10.086 | −4.481 | −6.232 | 1.00 | 10.00 | A | N |
| ATOM | 2051 | CZ | ARG | A | 225 | −9.034 | −5.259 | −6.517 | 1.00 | 10.00 | A | C |
| ATOM | 2052 | NH1 | ARG | A | 225 | −9.185 | −6.364 | −7.222 | 1.00 | 10.00 | A | N |
| ATOM | 2055 | NH2 | ARG | A | 225 | −7.826 | −4.939 | −6.074 | 1.00 | 10.00 | A | N |
| ATOM | 2058 | C | ARG | A | 225 | −11.948 | −1.313 | −9.754 | 1.00 | 10.00 | A | C |
| ATOM | 2059 | O | ARG | A | 225 | −13.021 | −0.749 | −9.527 | 1.00 | 10.00 | A | O |
| ATOM | 2060 | N | GLU | A | 226 | −10.790 | −0.660 | −9.832 | 1.00 | 10.00 | A | N |
| ATOM | 2062 | CA | GLU | A | 226 | −10.706 | 0.787 | −9.630 | 1.00 | 10.00 | A | C |
| ATOM | 2063 | CB | GLU | A | 226 | −9.258 | 1.270 | −9.707 | 1.00 | 10.00 | A | C |
| ATOM | 2064 | CG | GLU | A | 226 | −8.361 | 0.703 | −8.627 | 1.00 | 10.00 | A | C |
| ATOM | 2065 | CD | GLU | A | 226 | −8.913 | 0.929 | −7.235 | 1.00 | 10.00 | A | C |
| ATOM | 2066 | OE1 | GLU | A | 226 | −8.882 | −0.020 | −6.424 | 1.00 | 10.00 | A | O |
| ATOM | 2067 | OE2 | GLU | A | 226 | −9.355 | 2.061 | −6.939 | 1.00 | 10.00 | A | O |
| ATOM | 2068 | C | GLU | A | 226 | −11.558 | 1.545 | −10.641 | 1.00 | 10.00 | A | C |
| ATOM | 2069 | O | GLU | A | 226 | −12.276 | 2.483 | −10.283 | 1.00 | 10.00 | A | O |
| ATOM | 2070 | N | HIS | A | 227 | −11.479 | 1.121 | −11.899 | 1.00 | 10.00 | A | N |
| ATOM | 2072 | CA | HIS | A | 227 | −12.235 | 1.741 | −12.978 | 1.00 | 10.00 | A | C |
| ATOM | 2073 | CB | HIS | A | 227 | −11.923 | 1.030 | −14.299 | 1.00 | 10.00 | A | C |
| ATOM | 2074 | CG | HIS | A | 227 | −12.356 | 1.780 | −15.518 | 1.00 | 10.00 | A | C |
| ATOM | 2075 | ND1 | HIS | A | 227 | −11.494 | 2.612 | −16.196 | 1.00 | 10.00 | A | N |
| ATOM | 2076 | CD2 | HIS | A | 227 | −13.561 | 1.784 | −16.140 | 1.00 | 10.00 | A | C |
| ATOM | 2077 | CE1 | HIS | A | 227 | −12.188 | 3.099 | −17.210 | 1.00 | 10.00 | A | C |
| ATOM | 2078 | NE2 | HIS | A | 227 | −13.440 | 2.626 | −17.214 | 1.00 | 10.00 | A | N |
| ATOM | 2080 | C | HIS | A | 227 | −13.730 | 1.685 | −12.685 | 1.00 | 10.00 | A | C |
| ATOM | 2081 | O | HIS | A | 227 | −14.427 | 2.698 | −12.756 | 1.00 | 10.00 | A | O |
| ATOM | 2082 | N | TYR | A | 228 | −14.206 | 0.500 | −12.323 | 1.00 | 10.00 | A | N |
| ATOM | 2084 | CA | TYR | A | 228 | −15.619 | 0.295 | −12.014 | 1.00 | 10.00 | A | C |
| ATOM | 2085 | CB | TYR | A | 228 | −15.919 | −1.185 | −11.759 | 1.00 | 10.00 | A | C |
| ATOM | 2086 | CG | TYR | A | 228 | −16.162 | −2.003 | −13.012 | 1.00 | 10.00 | A | C |
| ATOM | 2087 | CD1 | TYR | A | 228 | −17.196 | −2.932 | −13.075 | 1.00 | 10.00 | A | C |
| ATOM | 2088 | CD2 | TYR | A | 228 | −15.353 | −1.854 | −14.132 | 1.00 | 10.00 | A | C |
| ATOM | 2089 | CE1 | TYR | A | 228 | −17.413 | −3.686 | −14.216 | 1.00 | 10.00 | A | C |
| ATOM | 2090 | CE2 | TYR | A | 228 | −15.564 | −2.601 | −15.275 | 1.00 | 10.00 | A | C |
| ATOM | 2091 | CZ | TYR | A | 228 | −16.594 | −3.516 | −15.311 | 1.00 | 10.00 | A | C |
| ATOM | 2092 | OH | TYR | A | 228 | −16.801 | −4.272 | −16.444 | 1.00 | 10.00 | A | O |
| ATOM | 2094 | C | TYR | A | 228 | −16.050 | 1.131 | −10.813 | 1.00 | 10.00 | A | C |
| ATOM | 2095 | O | TYR | A | 228 | −17.117 | 1.750 | −10.828 | 1.00 | 10.00 | A | O |
| ATOM | 2096 | N | ILE | A | 229 | −15.213 | 1.147 | −9.779 | 1.00 | 10.00 | A | N |
| ATOM | 2098 | CA | ILE | A | 229 | −15.500 | 1.912 | −8.572 | 1.00 | 10.00 | A | C |
| ATOM | 2099 | CB | ILE | A | 229 | −14.393 | 1.739 | −7.504 | 1.00 | 10.00 | A | C |
| ATOM | 2100 | CG1 | ILE | A | 229 | −14.396 | 0.308 | −6.958 | 1.00 | 10.00 | A | C |
| ATOM | 2101 | CG2 | ILE | A | 229 | −14.572 | 2.745 | −6.375 | 1.00 | 10.00 | A | C |
| ATOM | 2102 | CD1 | ILE | A | 229 | −13.233 | −0.010 | −6.042 | 1.00 | 10.00 | A | C |
| ATOM | 2103 | C | ILE | A | 229 | −15.678 | 3.395 | −8.894 | 1.00 | 10.00 | A | C |
| ATOM | 2104 | O | ILE | A | 229 | −16.685 | 3.999 | −8.530 | 1.00 | 10.00 | A | O |
| ATOM | 2105 | N | GLU | A | 230 | −14.710 | 3.966 | −9.605 | 1.00 | 10.00 | A | N |
| ATOM | 2107 | CA | GLU | A | 230 | −14.761 | 5.379 | −9.963 | 1.00 | 10.00 | A | C |
| ATOM | 2108 | CB | GLU | A | 230 | −13.477 | 5.815 | −10.660 | 1.00 | 10.00 | A | C |
| ATOM | 2109 | CG | GLU | A | 230 | −12.624 | 6.774 | −9.845 | 1.00 | 10.00 | A | C |
| ATOM | 2110 | CD | GLU | A | 230 | −13.328 | 8.085 | −9.551 | 1.00 | 10.00 | A | C |
| ATOM | 2111 | OE1 | GLU | A | 230 | −13.448 | 8.451 | −8.361 | 1.00 | 10.00 | A | O |
| ATOM | 2112 | OE2 | GLU | A | 230 | −13.752 | 8.767 | −10.505 | 1.00 | 10.00 | A | O |
| ATOM | 2113 | C | GLU | A | 230 | −15.978 | 5.702 | −10.827 | 1.00 | 10.00 | A | C |
| ATOM | 2114 | O | GLU | A | 230 | −16.481 | 6.825 | −10.806 | 1.00 | 10.00 | A | O |
| ATOM | 2115 | N | GLU | A | 231 | −16.445 | 4.716 | −11.593 | 1.00 | 10.00 | A | N |
| ATOM | 2117 | CA | GLU | A | 231 | −17.614 | 4.912 | −12.446 | 1.00 | 10.00 | A | C |
| ATOM | 2118 | CB | GLU | A | 231 | −17.776 | 3.786 | −13.477 | 1.00 | 10.00 | A | C |
| ATOM | 2119 | CG | GLU | A | 231 | −16.707 | 3.762 | −14.557 | 1.00 | 10.00 | A | C |
| ATOM | 2120 | CD | GLU | A | 231 | −17.204 | 3.214 | −15.884 | 1.00 | 10.00 | A | C |
| ATOM | 2121 | OE1 | GLU | A | 231 | −18.024 | 2.272 | −15.882 | 1.00 | 10.00 | A | O |
| ATOM | 2122 | OE2 | GLU | A | 231 | −16.773 | 3.728 | −16.938 | 1.00 | 10.00 | A | O |
| ATOM | 2123 | C | GLU | A | 231 | −18.872 | 5.038 | −11.597 | 1.00 | 10.00 | A | C |
| ATOM | 2124 | O | GLU | A | 231 | −19.633 | 5.993 | −11.747 | 1.00 | 10.00 | A | O |
| ATOM | 2125 | N | PHE | A | 232 | −19.067 | 4.084 | −10.688 | 1.00 | 10.00 | A | N |
| ATOM | 2127 | CA | PHE | A | 232 | −20.228 | 4.087 | −9.802 | 1.00 | 10.00 | A | C |
| ATOM | 2128 | CB | PHE | A | 232 | −20.260 | 2.829 | −8.922 | 1.00 | 10.00 | A | C |
| ATOM | 2129 | CG | PHE | A | 232 | −20.410 | 1.549 | −9.691 | 1.00 | 10.00 | A | C |
| ATOM | 2130 | CD1 | PHE | A | 232 | −21.314 | 1.454 | −10.737 | 1.00 | 10.00 | A | C |
| ATOM | 2131 | CD2 | PHE | A | 232 | −19.648 | 0.437 | −9.368 | 1.00 | 10.00 | A | C |
| ATOM | 2132 | CE1 | PHE | A | 232 | −21.454 | 0.278 | −11.448 | 1.00 | 10.00 | A | C |
| ATOM | 2133 | CE2 | PHE | A | 232 | −19.783 | −0.741 | −10.075 | 1.00 | 10.00 | A | C |
| ATOM | 2134 | CZ | PHE | A | 232 | −20.688 | −0.822 | −11.116 | 1.00 | 10.00 | A | C |
| ATOM | 2135 | C | PHE | A | 232 | −20.218 | 5.331 | −8.928 | 1.00 | 10.00 | A | C |
| ATOM | 2136 | O | PHE | A | 232 | −21.238 | 6.006 | −8.776 | 1.00 | 10.00 | A | O |
| ATOM | 2137 | N | VAL | A | 233 | −19.053 | 5.632 | −8.367 | 1.00 | 10.00 | A | N |
| ATOM | 2139 | CA | VAL | A | 233 | −18.892 | 6.801 | −7.518 | 1.00 | 10.00 | A | C |
| ATOM | 2140 | CB | VAL | A | 233 | −17.485 | 6.832 | −6.882 | 1.00 | 10.00 | A | C |
| ATOM | 2141 | CG1 | VAL | A | 233 | −17.234 | 8.151 | −6.176 | 1.00 | 10.00 | A | C |
| ATOM | 2142 | CG2 | VAL | A | 233 | −17.322 | 5.678 | −5.902 | 1.00 | 10.00 | A | C |
| ATOM | 2143 | C | VAL | A | 233 | −19.124 | 8.068 | −8.336 | 1.00 | 10.00 | A | C |
| ATOM | 2144 | O | VAL | A | 233 | −19.728 | 9.030 | −7.858 | 1.00 | 10.00 | A | O |
| ATOM | 2145 | N | GLY | A | 234 | −18.670 | 8.039 | −9.583 | 1.00 | 10.00 | A | N |
| ATOM | 2147 | CA | GLY | A | 234 | −18.833 | 9.171 | −10.468 | 1.00 | 10.00 | A | C |
| ATOM | 2148 | C | GLY | A | 234 | −20.290 | 9.494 | −10.711 | 1.00 | 10.00 | A | C |
| ATOM | 2149 | O | GLY | A | 234 | −20.685 | 10.662 | −10.677 | 1.00 | 10.00 | A | O |
| ATOM | 2150 | N | HIS | A | 235 | −21.083 | 8.459 | −10.956 | 1.00 | 10.00 | A | N |
| ATOM | 2152 | CA | HIS | A | 235 | −22.513 | 8.624 | −11.194 | 1.00 | 10.00 | A | C |
| ATOM | 2153 | CB | HIS | A | 235 | −23.170 | 7.275 | −11.498 | 1.00 | 10.00 | A | C |
| ATOM | 2154 | CG | HIS | A | 235 | −22.812 | 6.716 | −12.837 | 1.00 | 10.00 | A | C |
| ATOM | 2155 | ND1 | HIS | A | 235 | −22.401 | 5.412 | −12.982 | 1.00 | 10.00 | A | N |
| ATOM | 2156 | CD2 | HIS | A | 235 | −22.816 | 7.320 | −14.049 | 1.00 | 10.00 | A | C |
| ATOM | 2157 | CE1 | HIS | A | 235 | −22.164 | 5.250 | −14.272 | 1.00 | 10.00 | A | C |
| ATOM | 2158 | NE2 | HIS | A | 235 | −22.401 | 6.379 | −14.952 | 1.00 | 10.00 | A | N |
| ATOM | 2160 | C | HIS | A | 235 | −23.181 | 9.254 | −9.982 | 1.00 | 10.00 | A | C |
| ATOM | 2161 | O | HIS | A | 235 | −23.947 | 10.215 | −10.102 | 1.00 | 10.00 | A | O |
| ATOM | 2162 | N | LEU | A | 236 | −22.863 | 8.721 | −8.811 | 1.00 | 10.00 | A | N |
| ATOM | 2164 | CA | LEU | A | 236 | −23.421 | 9.221 | −7.568 | 1.00 | 10.00 | A | C |
| ATOM | 2165 | CB | LEU | A | 236 | −22.971 | 8.358 | −6.388 | 1.00 | 10.00 | A | C |
| ATOM | 2166 | CG | LEU | A | 236 | −23.291 | 6.865 | −6.483 | 1.00 | 10.00 | A | C |
| ATOM | 2167 | CD1 | LEU | A | 236 | −22.810 | 6.137 | −5.238 | 1.00 | 10.00 | A | C |
| ATOM | 2168 | CD2 | LEU | A | 236 | −24.783 | 6.649 | −6.689 | 1.00 | 10.00 | A | C |
| ATOM | 2169 | C | LEU | A | 236 | −23.026 | 10.671 | −7.340 | 1.00 | 10.00 | A | C |
| ATOM | 2170 | O | LEU | A | 236 | −23.854 | 11.483 | −6.941 | 1.00 | 10.00 | A | O |
| ATOM | 2171 | N | ARG | A | 237 | −21.761 | 10.998 | −7.603 | 1.00 | 10.00 | A | N |
| ATOM | 2173 | CA | ARG | A | 237 | −21.275 | 12.365 | −7.424 | 1.00 | 10.00 | A | C |
| ATOM | 2174 | CB | ARG | A | 237 | −19.768 | 12.471 | −7.696 | 1.00 | 10.00 | A | C |
| ATOM | 2175 | CG | ARG | A | 237 | −18.881 | 11.798 | −6.656 | 1.00 | 10.00 | A | C |
| ATOM | 2176 | CD | ARG | A | 237 | −17.400 | 12.017 | −6.951 | 1.00 | 10.00 | A | C |
| ATOM | 2177 | NE | ARG | A | 237 | −17.032 | 11.582 | −8.300 | 1.00 | 10.00 | A | N |
| ATOM | 2179 | CZ | ARG | A | 237 | −16.031 | 10.744 | −8.574 | 1.00 | 10.00 | A | C |
| ATOM | 2180 | NH1 | ARG | A | 237 | −15.281 | 10.251 | −7.594 | 1.00 | 10.00 | A | N |
| ATOM | 2183 | NH2 | ARG | A | 237 | −15.771 | 10.402 | −9.833 | 1.00 | 10.00 | A | N |
| ATOM | 2186 | C | ARG | A | 237 | −22.038 | 13.346 | −8.315 | 1.00 | 10.00 | A | C |
| ATOM | 2187 | O | ARG | A | 237 | −22.405 | 14.438 | −7.878 | 1.00 | 10.00 | A | O |
| ATOM | 2188 | N | ALA | A | 238 | −22.293 | 12.936 | −9.555 | 1.00 | 10.00 | A | N |
| ATOM | 2190 | CA | ALA | A | 238 | −23.005 | 13.775 | −10.513 | 1.00 | 10.00 | A | C |
| ATOM | 2191 | CB | ALA | A | 238 | −22.913 | 13.189 | −11.915 | 1.00 | 10.00 | A | C |
| ATOM | 2192 | C | ALA | A | 238 | −24.463 | 13.972 | −10.105 | 1.00 | 10.00 | A | C |
| ATOM | 2193 | O | ALA | A | 238 | −24.972 | 15.093 | −10.114 | 1.00 | 10.00 | A | O |
| ATOM | 2194 | N | GLU | A | 239 | −25.128 | 12.878 | −9.743 | 1.00 | 10.00 | A | N |
| ATOM | 2196 | CA | GLU | A | 239 | −26.529 | 12.932 | −9.335 | 1.00 | 10.00 | A | C |
| ATOM | 2197 | CB | GLU | A | 239 | −27.081 | 11.528 | −9.128 | 1.00 | 10.00 | A | C |
| ATOM | 2198 | CG | GLU | A | 239 | −27.196 | 10.704 | −10.396 | 1.00 | 10.00 | A | C |
| ATOM | 2199 | CD | GLU | A | 239 | −28.240 | 11.242 | −11.351 | 1.00 | 10.00 | A | C |
| ATOM | 2200 | OE1 | GLU | A | 239 | −29.413 | 11.384 | −10.943 | 1.00 | 10.00 | A | O |
| ATOM | 2201 | OE2 | GLU | A | 239 | −27.894 | 11.517 | −12.519 | 1.00 | 10.00 | A | O |
| ATOM | 2202 | C | GLU | A | 239 | −26.696 | 13.728 | −8.049 | 1.00 | 10.00 | A | C |
| ATOM | 2203 | O | GLU | A | 239 | −27.598 | 14.559 | −7.929 | 1.00 | 10.00 | A | O |
| ATOM | 2204 | N | MET | A | 240 | −25.814 | 13.462 | −7.093 | 1.00 | 10.00 | A | N |
| ATOM | 2206 | CA | MET | A | 240 | −25.830 | 14.136 | −5.802 | 1.00 | 10.00 | A | C |
| ATOM | 2207 | CB | MET | A | 240 | −24.700 | 13.585 | −4.927 | 1.00 | 10.00 | A | C |
| ATOM | 2208 | CG | MET | A | 240 | −24.572 | 14.223 | −3.559 | 1.00 | 10.00 | A | C |
| ATOM | 2209 | SD | MET | A | 240 | −23.094 | 13.679 | −2.684 | 1.00 | 10.00 | A | S |
| ATOM | 2210 | CE | MET | A | 240 | −21.811 | 14.215 | −3.818 | 1.00 | 10.00 | A | C |
| ATOM | 2211 | C | MET | A | 240 | −25.677 | 15.643 | −5.972 | 1.00 | 10.00 | A | C |
| ATOM | 2212 | O | MET | A | 240 | −26.418 | 16.426 | −5.374 | 1.00 | 10.00 | A | O |
| ATOM | 2213 | N | LYS | A | 241 | −24.726 | 16.040 | −6.809 | 1.00 | 10.00 | A | N |
| ATOM | 2215 | CA | LYS | A | 241 | −24.464 | 17.451 | −7.061 | 1.00 | 10.00 | A | C |
| ATOM | 2216 | CB | LYS | A | 241 | −23.192 | 17.621 | −7.896 | 1.00 | 10.00 | A | C |
| ATOM | 2217 | CG | LYS | A | 241 | −22.660 | 19.044 | −7.951 | 1.00 | 10.00 | A | C |
| ATOM | 2218 | CD | LYS | A | 241 | −21.334 | 19.101 | −8.695 | 1.00 | 10.00 | A | C |
| ATOM | 2219 | CE | LYS | A | 241 | −20.764 | 20.510 | −8.719 | 1.00 | 10.00 | A | C |
| ATOM | 2220 | NZ | LYS | A | 241 | −19.435 | 20.558 | −9.391 | 1.00 | 10.00 | A | N |
| ATOM | 2224 | C | LYS | A | 241 | −25.644 | 18.112 | −7.762 | 1.00 | 10.00 | A | C |
| ATOM | 2225 | O | LYS | A | 241 | −25.937 | 19.288 | −7.532 | 1.00 | 10.00 | A | O |
| ATOM | 2226 | N | ALA | A | 242 | −26.328 | 17.355 | −8.614 | 1.00 | 10.00 | A | N |
| ATOM | 2228 | CA | ALA | A | 242 | −27.478 | 17.880 | −9.342 | 1.00 | 10.00 | A | C |
| ATOM | 2229 | CB | ALA | A | 242 | −27.888 | 16.940 | −10.464 | 1.00 | 10.00 | A | C |
| ATOM | 2230 | C | ALA | A | 242 | −28.644 | 18.143 | −8.397 | 1.00 | 10.00 | A | C |
| ATOM | 2231 | O | ALA | A | 242 | −29.401 | 19.095 | −8.580 | 1.00 | 10.00 | A | O |
| ATOM | 2232 | N | GLU | A | 243 | −28.788 | 17.288 | −7.392 | 1.00 | 10.00 | A | N |
| ATOM | 2234 | CA | GLU | A | 243 | −29.851 | 17.440 | −6.408 | 1.00 | 10.00 | A | C |
| ATOM | 2235 | CB | GLU | A | 243 | −30.088 | 16.133 | −5.653 | 1.00 | 10.00 | A | C |
| ATOM | 2236 | CG | GLU | A | 243 | −30.772 | 15.056 | −6.481 | 1.00 | 10.00 | A | C |
| ATOM | 2237 | CD | GLU | A | 243 | −32.110 | 15.505 | −7.039 | 1.00 | 10.00 | A | C |
| ATOM | 2238 | OE1 | GLU | A | 243 | −33.139 | 15.329 | −6.351 | 1.00 | 10.00 | A | O |
| ATOM | 2239 | OE2 | GLU | A | 243 | −32.141 | 16.030 | −8.167 | 1.00 | 10.00 | A | O |
| ATOM | 2240 | C | GLU | A | 243 | −29.522 | 18.565 | −5.435 | 1.00 | 10.00 | A | C |
| ATOM | 2241 | O | GLU | A | 243 | −30.415 | 19.202 | −4.877 | 1.00 | 10.00 | A | O |
| ATOM | 2242 | N | GLY | A | 244 | −28.233 | 18.797 | −5.228 | 1.00 | 10.00 | A | N |
| ATOM | 2244 | CA | GLY | A | 244 | −27.807 | 19.861 | −4.342 | 1.00 | 10.00 | A | C |
| ATOM | 2245 | C | GLY | A | 244 | −27.165 | 19.362 | −3.065 | 1.00 | 10.00 | A | C |
| ATOM | 2246 | O | GLY | A | 244 | −26.864 | 20.153 | −2.169 | 1.00 | 10.00 | A | O |
| ATOM | 2247 | N | VAL | A | 245 | −26.946 | 18.059 | −2.979 | 1.00 | 10.00 | A | N |
| ATOM | 2249 | CA | VAL | A | 245 | −26.333 | 17.463 | −1.796 | 1.00 | 10.00 | A | C |
| ATOM | 2250 | CB | VAL | A | 245 | −26.785 | 15.997 | −1.597 | 1.00 | 10.00 | A | C |
| ATOM | 2251 | CG1 | VAL | A | 245 | −26.406 | 15.496 | −0.209 | 1.00 | 10.00 | A | C |
| ATOM | 2252 | CG2 | VAL | A | 245 | −28.286 | 15.861 | −1.820 | 1.00 | 10.00 | A | C |
| ATOM | 2253 | C | VAL | A | 245 | −24.805 | 17.524 | −1.900 | 1.00 | 10.00 | A | C |
| ATOM | 2254 | O | VAL | A | 245 | −24.236 | 17.318 | −2.976 | 1.00 | 10.00 | A | O |
| ATOM | 2255 | N | LYS | A | 246 | −24.148 | 17.829 | −0.792 | 1.00 | 10.00 | A | N |
| ATOM | 2257 | CA | LYS | A | 246 | −22.692 | 17.922 | −0.765 | 1.00 | 10.00 | A | C |
| ATOM | 2258 | CB | LYS | A | 246 | −22.237 | 19.317 | −0.327 | 1.00 | 10.00 | A | C |
| ATOM | 2259 | CG | LYS | A | 246 | −22.831 | 20.462 | −1.128 | 1.00 | 10.00 | A | C |
| ATOM | 2260 | CD | LYS | A | 246 | −23.929 | 21.167 | −0.348 | 1.00 | 10.00 | A | C |
| ATOM | 2261 | CE | LYS | A | 246 | −24.343 | 22.474 | −1.010 | 1.00 | 10.00 | A | C |
| ATOM | 2262 | NZ | LYS | A | 246 | −25.103 | 22.252 | −2.269 | 1.00 | 10.00 | A | N |
| ATOM | 2266 | C | LYS | A | 246 | −22.095 | 16.875 | 0.165 | 1.00 | 10.00 | A | C |
| ATOM | 2267 | O | LYS | A | 246 | −22.101 | 17.042 | 1.386 | 1.00 | 10.00 | A | O |
| ATOM | 2268 | N | ALA | A | 247 | −21.581 | 15.799 | −0.412 | 1.00 | 10.00 | A | N |
| ATOM | 2270 | CA | ALA | A | 247 | −20.972 | 14.729 | 0.367 | 1.00 | 10.00 | A | C |
| ATOM | 2271 | CB | ALA | A | 247 | −21.953 | 13.587 | 0.559 | 1.00 | 10.00 | A | C |
| ATOM | 2272 | C | ALA | A | 247 | −19.700 | 14.228 | −0.308 | 1.00 | 10.00 | A | C |
| ATOM | 2273 | O | ALA | A | 247 | −19.483 | 14.463 | −1.500 | 1.00 | 10.00 | A | O |
| ATOM | 2274 | N | GLU | A | 248 | −18.863 | 13.541 | 0.457 | 1.00 | 10.00 | A | N |
| ATOM | 2276 | CA | GLU | A | 248 | −17.615 | 13.005 | −0.065 | 1.00 | 10.00 | A | C |
| ATOM | 2277 | CB | GLU | A | 248 | −16.497 | 13.162 | 0.960 | 1.00 | 10.00 | A | C |
| ATOM | 2278 | CG | GLU | A | 248 | −16.303 | 14.585 | 1.446 | 1.00 | 10.00 | A | C |
| ATOM | 2279 | CD | GLU | A | 248 | −14.919 | 14.814 | 2.000 | 1.00 | 10.00 | A | C |
| ATOM | 2280 | OE1 | GLU | A | 248 | −14.692 | 14.498 | 3.183 | 1.00 | 10.00 | A | O |
| ATOM | 2281 | OE2 | GLU | A | 248 | −14.051 | 15.309 | 1.250 | 1.00 | 10.00 | A | O |
| ATOM | 2282 | C | GLU | A | 248 | −17.775 | 11.541 | −0.443 | 1.00 | 10.00 | A | C |
| ATOM | 2283 | O | GLU | A | 248 | −17.858 | 10.669 | 0.420 | 1.00 | 10.00 | A | O |
| ATOM | 2284 | N | VAL | A | 249 | −17.833 | 11.277 | −1.733 | 1.00 | 10.00 | A | N |
| ATOM | 2286 | CA | VAL | A | 249 | −17.989 | 9.914 | −2.218 | 1.00 | 10.00 | A | C |
| ATOM | 2287 | CB | VAL | A | 249 | −19.133 | 9.799 | −3.252 | 1.00 | 10.00 | A | C |
| ATOM | 2288 | CG1 | VAL | A | 249 | −19.498 | 8.339 | −3.477 | 1.00 | 10.00 | A | C |
| ATOM | 2289 | CG2 | VAL | A | 249 | −20.353 | 10.594 | −2.806 | 1.00 | 10.00 | A | C |
| ATOM | 2290 | C | VAL | A | 249 | −16.692 | 9.417 | −2.847 | 1.00 | 10.00 | A | C |
| ATOM | 2291 | O | VAL | A | 249 | −16.158 | 10.042 | −3.764 | 1.00 | 10.00 | A | O |
| ATOM | 2292 | N | TYR | A | 250 | −16.184 | 8.298 | −2.341 | 1.00 | 10.00 | A | N |
| ATOM | 2294 | CA | TYR | A | 250 | −14.955 | 7.709 | −2.854 | 1.00 | 10.00 | A | C |
| ATOM | 2295 | CB | TYR | A | 250 | −13.711 | 8.451 | −2.338 | 1.00 | 10.00 | A | C |
| ATOM | 2296 | CG | TYR | A | 250 | −13.655 | 8.661 | −0.835 | 1.00 | 10.00 | A | C |
| ATOM | 2297 | CD1 | TYR | A | 250 | −13.371 | 7.609 | 0.030 | 1.00 | 10.00 | A | C |
| ATOM | 2298 | CD2 | TYR | A | 250 | −13.876 | 9.916 | −0.281 | 1.00 | 10.00 | A | C |
| ATOM | 2299 | CE1 | TYR | A | 250 | −13.314 | 7.799 | 1.399 | 1.00 | 10.00 | A | C |
| ATOM | 2300 | CE2 | TYR | A | 250 | −13.820 | 10.115 | 1.085 | 1.00 | 10.00 | A | C |
| ATOM | 2301 | CZ | TYR | A | 250 | −13.540 | 9.053 | 1.921 | 1.00 | 10.00 | A | C |
| ATOM | 2302 | OH | TYR | A | 250 | −13.480 | 9.248 | 3.287 | 1.00 | 10.00 | A | O |
| ATOM | 2304 | C | TYR | A | 250 | −14.884 | 6.225 | −2.523 | 1.00 | 10.00 | A | C |
| ATOM | 2305 | O | TYR | A | 250 | −15.519 | 5.753 | −1.577 | 1.00 | 10.00 | A | O |
| ATOM | 2306 | N | GLY | A | 251 | −14.130 | 5.490 | −3.318 | 1.00 | 10.00 | A | N |
| ATOM | 2308 | CA | GLY | A | 251 | −13.989 | 4.070 | −3.095 | 1.00 | 10.00 | A | C |
| ATOM | 2309 | C | GLY | A | 251 | −12.757 | 3.729 | −2.279 | 1.00 | 10.00 | A | C |
| ATOM | 2310 | O | GLY | A | 251 | −11.644 | 4.130 | −2.632 | 1.00 | 10.00 | A | O |
| ATOM | 2311 | N | ARG | A | 252 | −12.949 | 2.998 | −1.189 | 1.00 | 10.00 | A | N |
| ATOM | 2313 | CA | ARG | A | 252 | −11.843 | 2.599 | −0.331 | 1.00 | 10.00 | A | C |
| ATOM | 2314 | CB | ARG | A | 252 | −12.041 | 3.123 | 1.093 | 1.00 | 10.00 | A | C |
| ATOM | 2315 | CG | ARG | A | 252 | −10.743 | 3.330 | 1.864 | 1.00 | 10.00 | A | C |
| ATOM | 2316 | CD | ARG | A | 252 | −10.856 | 2.846 | 3.303 | 1.00 | 10.00 | A | C |
| ATOM | 2317 | NE | ARG | A | 252 | −10.845 | 1.384 | 3.391 | 1.00 | 10.00 | A | N |
| ATOM | 2319 | CZ | ARG | A | 252 | −10.770 | 0.696 | 4.534 | 1.00 | 10.00 | A | C |
| ATOM | 2320 | NH1 | ARG | A | 252 | −10.692 | 1.330 | 5.701 | 1.00 | 10.00 | A | N |
| ATOM | 2323 | NH2 | ARG | A | 252 | −10.775 | −0.633 | 4.497 | 1.00 | 10.00 | A | N |
| ATOM | 2326 | C | ARG | A | 252 | −11.712 | 1.082 | −0.319 | 1.00 | 10.00 | A | C |
| ATOM | 2327 | O | ARG | A | 252 | −12.671 | 0.373 | −0.014 | 1.00 | 10.00 | A | O |
| ATOM | 2328 | N | PRO | A | 253 | −10.524 | 0.568 | −0.660 | 1.00 | 10.00 | A | N |
| ATOM | 2329 | CA | PRO | A | 253 | −10.250 | −0.875 | −0.695 | 1.00 | 10.00 | A | C |
| ATOM | 2330 | CB | PRO | A | 253 | −8.785 | −0.957 | −1.135 | 1.00 | 10.00 | A | C |
| ATOM | 2331 | CG | PRO | A | 253 | −8.522 | 0.347 | −1.803 | 1.00 | 10.00 | A | C |
| ATOM | 2332 | CD | PRO | A | 253 | −9.349 | 1.352 | −1.059 | 1.00 | 10.00 | A | C |
| ATOM | 2333 | C | PRO | A | 253 | −10.415 | −1.536 | 0.674 | 1.00 | 10.00 | A | C |
| ATOM | 2334 | O | PRO | A | 253 | −10.575 | −0.858 | 1.693 | 1.00 | 10.00 | A | O |
| ATOM | 2335 | N | LYS | A | 254 | −10.365 | −2.861 | 0.692 | 1.00 | 10.00 | A | N |
| ATOM | 2337 | CA | LYS | A | 254 | −10.505 | −3.620 | 1.922 | 1.00 | 10.00 | A | C |
| ATOM | 2338 | CB | LYS | A | 254 | −11.662 | −4.618 | 1.804 | 1.00 | 10.00 | A | C |
| ATOM | 2339 | CG | LYS | A | 254 | −13.020 | −3.950 | 1.657 | 1.00 | 10.00 | A | C |
| ATOM | 2340 | CD | LYS | A | 254 | −14.119 | −4.954 | 1.352 | 1.00 | 10.00 | A | C |
| ATOM | 2341 | CE | LYS | A | 254 | −15.476 | −4.271 | 1.275 | 1.00 | 10.00 | A | C |
| ATOM | 2342 | NZ | LYS | A | 254 | −16.559 | −5.227 | 0.934 | 1.00 | 10.00 | A | N |
| ATOM | 2346 | C | LYS | A | 254 | −9.197 | −4.334 | 2.237 | 1.00 | 10.00 | A | C |
| ATOM | 2347 | O | LYS | A | 254 | −8.494 | −4.782 | 1.331 | 1.00 | 10.00 | A | O |
| ATOM | 2348 | N | HIS | A | 255 | −8.863 | −4.416 | 3.516 | 1.00 | 10.00 | A | N |
| ATOM | 2350 | CA | HIS | A | 255 | −7.627 | −5.058 | 3.949 | 1.00 | 10.00 | A | C |
| ATOM | 2351 | CB | HIS | A | 255 | −7.329 | −4.732 | 5.413 | 1.00 | 10.00 | A | C |
| ATOM | 2352 | CG | HIS | A | 255 | −6.975 | −3.296 | 5.674 | 1.00 | 10.00 | A | C |
| ATOM | 2353 | ND1 | HIS | A | 255 | −6.068 | −2.959 | 6.648 | 1.00 | 10.00 | A | N |
| ATOM | 2354 | CD2 | HIS | A | 255 | −7.438 | −2.163 | 5.084 | 1.00 | 10.00 | A | C |
| ATOM | 2355 | CE1 | HIS | A | 255 | −5.995 | −1.638 | 6.635 | 1.00 | 10.00 | A | C |
| ATOM | 2356 | NE2 | HIS | A | 255 | −6.806 | −1.116 | 5.708 | 1.00 | 10.00 | A | N |
| ATOM | 2358 | C | HIS | A | 255 | −7.664 | −6.568 | 3.733 | 1.00 | 10.00 | A | C |
| ATOM | 2359 | O | HIS | A | 255 | −8.736 | −7.178 | 3.705 | 1.00 | 10.00 | A | O |
| ATOM | 2360 | N | ILE | A | 256 | −6.482 | −7.162 | 3.595 | 1.00 | 10.00 | A | N |
| ATOM | 2362 | CA | ILE | A | 256 | −6.357 | −8.596 | 3.366 | 1.00 | 10.00 | A | C |
| ATOM | 2363 | CB | ILE | A | 256 | −4.887 | −9.037 | 3.210 | 1.00 | 10.00 | A | C |
| ATOM | 2364 | CG1 | ILE | A | 256 | −4.222 | −8.284 | 2.057 | 1.00 | 10.00 | A | C |
| ATOM | 2365 | CG2 | ILE | A | 256 | −4.787 | −10.541 | 2.993 | 1.00 | 10.00 | A | C |
| ATOM | 2366 | CD1 | ILE | A | 256 | −4.861 | −8.524 | 0.704 | 1.00 | 10.00 | A | C |
| ATOM | 2367 | C | ILE | A | 256 | −7.045 | −9.419 | 4.456 | 1.00 | 10.00 | A | C |
| ATOM | 2368 | O | ILE | A | 256 | −7.757 | −10.369 | 4.155 | 1.00 | 10.00 | A | O |
| ATOM | 2369 | N | TYR | A | 257 | −6.848 | −9.046 | 5.717 | 1.00 | 10.00 | A | N |
| ATOM | 2371 | CA | TYR | A | 257 | −7.474 | −9.775 | 6.821 | 1.00 | 10.00 | A | C |
| ATOM | 2372 | CB | TYR | A | 257 | −6.929 | −9.330 | 8.189 | 1.00 | 10.00 | A | C |
| ATOM | 2373 | CG | TYR | A | 257 | −7.265 | −7.911 | 8.593 | 1.00 | 10.00 | A | C |
| ATOM | 2374 | CD1 | TYR | A | 257 | −6.409 | −6.862 | 8.289 | 1.00 | 10.00 | A | C |
| ATOM | 2375 | CD2 | TYR | A | 257 | −8.433 | −7.621 | 9.289 | 1.00 | 10.00 | A | C |
| ATOM | 2376 | CE1 | TYR | A | 257 | −6.706 | −5.567 | 8.663 | 1.00 | 10.00 | A | C |
| ATOM | 2377 | CE2 | TYR | A | 257 | −8.739 | −6.329 | 9.664 | 1.00 | 10.00 | A | C |
| ATOM | 2378 | CZ | TYR | A | 257 | −7.873 | −5.306 | 9.347 | 1.00 | 10.00 | A | C |
| ATOM | 2379 | OH | TYR | A | 257 | −8.176 | −4.011 | 9.717 | 1.00 | 10.00 | A | O |
| ATOM | 2381 | C | TYR | A | 257 | −9.001 | −9.689 | 6.763 | 1.00 | 10.00 | A | C |
| ATOM | 2382 | O | TYR | A | 257 | −9.702 | −10.598 | 7.208 | 1.00 | 10.00 | A | O |
| ATOM | 2383 | N | SER | A | 258 | −9.506 | −8.604 | 6.185 | 1.00 | 10.00 | A | N |
| ATOM | 2385 | CA | SER | A | 258 | −10.937 | −8.399 | 6.059 | 1.00 | 10.00 | A | C |
| ATOM | 2386 | CB | SER | A | 258 | −11.226 | −6.927 | 5.774 | 1.00 | 10.00 | A | C |
| ATOM | 2387 | OG | SER | A | 258 | −10.380 | −6.093 | 6.557 | 1.00 | 10.00 | A | O |
| ATOM | 2389 | C | SER | A | 258 | −11.514 | −9.285 | 4.954 | 1.00 | 10.00 | A | C |
| ATOM | 2390 | O | SER | A | 258 | −12.558 | −9.915 | 5.129 | 1.00 | 10.00 | A | O |
| ATOM | 2391 | N | ILE | A | 259 | −10.819 | −9.353 | 3.822 | 1.00 | 10.00 | A | N |
| ATOM | 2393 | CA | ILE | A | 259 | −11.274 | −10.174 | 2.704 | 1.00 | 10.00 | A | C |
| ATOM | 2394 | CB | ILE | A | 259 | −10.678 | −9.729 | 1.354 | 1.00 | 10.00 | A | C |
| ATOM | 2395 | CG1 | ILE | A | 259 | −9.151 | −9.626 | 1.437 | 1.00 | 10.00 | A | C |
| ATOM | 2396 | CG2 | ILE | A | 259 | −11.290 | −8.405 | 0.923 | 1.00 | 10.00 | A | C |
| ATOM | 2397 | CD1 | ILE | A | 259 | −8.487 | −9.222 | 0.139 | 1.00 | 10.00 | A | C |
| ATOM | 2398 | C | ILE | A | 259 | −10.987 | −11.654 | 2.954 | 1.00 | 10.00 | A | C |
| ATOM | 2399 | O | ILE | A | 259 | −11.672 | −12.524 | 2.422 | 1.00 | 10.00 | A | O |
| ATOM | 2400 | N | TRP | A | 260 | −9.984 | −11.921 | 3.785 | 1.00 | 10.00 | A | N |
| ATOM | 2402 | CA | TRP | A | 260 | −9.601 | −13.286 | 4.132 | 1.00 | 10.00 | A | C |
| ATOM | 2403 | CB | TRP | A | 260 | −8.391 | −13.270 | 5.078 | 1.00 | 10.00 | A | C |
| ATOM | 2404 | CG | TRP | A | 260 | −7.919 | −14.622 | 5.527 | 1.00 | 10.00 | A | C |
| ATOM | 2405 | CD1 | TRP | A | 260 | −7.952 | −15.791 | 4.819 | 1.00 | 10.00 | A | C |
| ATOM | 2406 | CD2 | TRP | A | 260 | −7.316 | −14.940 | 6.789 | 1.00 | 10.00 | A | C |
| ATOM | 2407 | NE1 | TRP | A | 260 | −7.421 | −16.813 | 5.567 | 1.00 | 10.00 | A | N |
| ATOM | 2409 | CE2 | TRP | A | 260 | −7.019 | −16.317 | 6.780 | 1.00 | 10.00 | A | C |
| ATOM | 2410 | CE3 | TRP | A | 260 | −7.002 | −14.191 | 7.927 | 1.00 | 10.00 | A | C |
| ATOM | 2411 | CZ2 | TRP | A | 260 | −6.423 | −16.958 | 7.862 | 1.00 | 10.00 | A | C |
| ATOM | 2412 | CZ3 | TRP | A | 260 | −6.409 | −14.829 | 8.998 | 1.00 | 10.00 | A | C |
| ATOM | 2413 | CH2 | TRP | A | 260 | −6.126 | −16.198 | 8.960 | 1.00 | 10.00 | A | C |
| ATOM | 2414 | C | TRP | A | 260 | −10.773 | −14.021 | 4.776 | 1.00 | 10.00 | A | C |
| ATOM | 2415 | O | TRP | A | 260 | −11.156 | −15.105 | 4.330 | 1.00 | 10.00 | A | O |
| ATOM | 2416 | N | ARG | A | 261 | −11.349 | −13.420 | 5.815 | 1.00 | 10.00 | A | N |
| ATOM | 2418 | CA | ARG | A | 261 | −12.484 | −14.026 | 6.501 | 1.00 | 10.00 | A | C |
| ATOM | 2419 | CB | ARG | A | 261 | −12.781 | −13.331 | 7.836 | 1.00 | 10.00 | A | C |
| ATOM | 2420 | CG | ARG | A | 261 | −13.155 | −11.858 | 7.726 | 1.00 | 10.00 | A | C |
| ATOM | 2421 | CD | ARG | A | 261 | −13.301 | −11.220 | 9.102 | 1.00 | 10.00 | A | C |
| ATOM | 2422 | NE | ARG | A | 261 | −13.922 | −9.897 | 9.033 | 1.00 | 10.00 | A | N |
| ATOM | 2424 | CZ | ARG | A | 261 | −13.736 | −8.933 | 9.938 | 1.00 | 10.00 | A | C |
| ATOM | 2425 | NH1 | ARG | A | 261 | −12.943 | −9.139 | 10.985 | 1.00 | 10.00 | A | N |
| ATOM | 2428 | NH2 | ARG | A | 261 | −14.348 | −7.759 | 9.799 | 1.00 | 10.00 | A | N |
| ATOM | 2431 | C | ARG | A | 261 | −13.712 | −14.052 | 5.595 | 1.00 | 10.00 | A | C |
| ATOM | 2432 | O | ARG | A | 261 | −14.572 | −14.923 | 5.722 | 1.00 | 10.00 | A | O |
| ATOM | 2433 | N | LYS | A | 262 | −13.773 | −13.105 | 4.664 | 1.00 | 10.00 | A | N |
| ATOM | 2435 | CA | LYS | A | 262 | −14.880 | −13.030 | 3.720 | 1.00 | 10.00 | A | C |
| ATOM | 2436 | CB | LYS | A | 262 | −14.823 | −11.717 | 2.937 | 1.00 | 10.00 | A | C |
| ATOM | 2437 | CG | LYS | A | 262 | −15.601 | −10.567 | 3.557 | 1.00 | 10.00 | A | C |
| ATOM | 2438 | CD | LYS | A | 262 | −17.101 | −10.810 | 3.482 | 1.00 | 10.00 | A | C |
| ATOM | 2439 | CE | LYS | A | 262 | −17.885 | −9.506 | 3.580 | 1.00 | 10.00 | A | C |
| ATOM | 2440 | NZ | LYS | A | 262 | −18.434 | −9.073 | 2.262 | 1.00 | 10.00 | A | N |
| ATOM | 2444 | C | LYS | A | 262 | −14.812 | −14.201 | 2.748 | 1.00 | 10.00 | A | C |
| ATOM | 2445 | O | LYS | A | 262 | −15.808 | −14.884 | 2.508 | 1.00 | 10.00 | A | O |
| ATOM | 2446 | N | MET | A | 263 | −13.620 | −14.428 | 2.203 | 1.00 | 10.00 | A | N |
| ATOM | 2448 | CA | MET | A | 263 | −13.383 | −15.514 | 1.260 | 1.00 | 10.00 | A | C |
| ATOM | 2449 | CB | MET | A | 263 | −11.973 | −15.396 | 0.660 | 1.00 | 10.00 | A | C |
| ATOM | 2450 | CG | MET | A | 263 | −11.531 | −16.597 | −0.160 | 1.00 | 10.00 | A | C |
| ATOM | 2451 | SD | MET | A | 263 | −10.167 | −16.229 | −1.282 | 1.00 | 10.00 | A | S |
| ATOM | 2452 | CE | MET | A | 263 | −8.873 | −15.765 | −0.128 | 1.00 | 10.00 | A | C |
| ATOM | 2453 | C | MET | A | 263 | −13.575 | −16.875 | 1.924 | 1.00 | 10.00 | A | C |
| ATOM | 2454 | O | MET | A | 263 | −13.977 | −17.842 | 1.275 | 1.00 | 10.00 | A | O |
| ATOM | 2455 | N | GLN | A | 264 | −13.291 | −16.941 | 3.220 | 1.00 | 10.00 | A | N |
| ATOM | 2457 | CA | GLN | A | 264 | −13.435 | −18.176 | 3.972 | 1.00 | 10.00 | A | C |
| ATOM | 2458 | CB | GLN | A | 264 | −12.668 | −18.072 | 5.294 | 1.00 | 10.00 | A | C |
| ATOM | 2459 | CG | GLN | A | 264 | −12.707 | −19.328 | 6.149 | 1.00 | 10.00 | A | C |
| ATOM | 2460 | CD | GLN | A | 264 | −11.894 | −20.470 | 5.567 | 1.00 | 10.00 | A | C |
| ATOM | 2461 | OE1 | GLN | A | 264 | −11.774 | −20.612 | 4.347 | 1.00 | 10.00 | A | O |
| ATOM | 2462 | NE2 | GLN | A | 264 | −11.332 | −21.291 | 6.436 | 1.00 | 10.00 | A | N |
| ATOM | 2465 | C | GLN | A | 264 | −14.907 | −18.483 | 4.240 | 1.00 | 10.00 | A | C |
| ATOM | 2466 | O | GLN | A | 264 | −15.384 | −19.597 | 3.993 | 1.00 | 10.00 | A | O |
| ATOM | 2467 | N | LYS | A | 265 | −15.624 | −17.482 | 4.726 | 1.00 | 10.00 | A | N |
| ATOM | 2469 | CA | LYS | A | 265 | −17.036 | −17.634 | 5.042 | 1.00 | 10.00 | A | C |
| ATOM | 2470 | CB | LYS | A | 265 | −17.528 | −16.450 | 5.879 | 1.00 | 10.00 | A | C |
| ATOM | 2471 | CG | LYS | A | 265 | −17.469 | −16.669 | 7.385 | 1.00 | 10.00 | A | C |
| ATOM | 2472 | CD | LYS | A | 265 | −16.084 | −17.101 | 7.836 | 1.00 | 10.00 | A | C |
| ATOM | 2473 | CE | LYS | A | 265 | −16.072 | −17.458 | 9.309 | 1.00 | 10.00 | A | C |
| ATOM | 2474 | NZ | LYS | A | 265 | −14.849 | −18.213 | 9.686 | 1.00 | 10.00 | A | N |
| ATOM | 2478 | C | LYS | A | 265 | −17.885 | −17.795 | 3.785 | 1.00 | 10.00 | A | C |
| ATOM | 2479 | O | LYS | A | 265 | −18.791 | −18.626 | 3.742 | 1.00 | 10.00 | A | O |
| ATOM | 2480 | N | LYS | A | 266 | −17.588 | −17.011 | 2.761 | 1.00 | 10.00 | A | N |
| ATOM | 2482 | CA | LYS | A | 266 | −18.330 | −17.081 | 1.509 | 1.00 | 10.00 | A | C |
| ATOM | 2483 | CB | LYS | A | 266 | −18.806 | −15.690 | 1.077 | 1.00 | 10.00 | A | C |
| ATOM | 2484 | CG | LYS | A | 266 | −19.921 | −15.707 | 0.041 | 1.00 | 10.00 | A | C |
| ATOM | 2485 | CD | LYS | A | 266 | −20.618 | −14.358 | −0.053 | 1.00 | 10.00 | A | C |
| ATOM | 2486 | CE | LYS | A | 266 | −19.670 | −13.271 | −0.530 | 1.00 | 10.00 | A | C |
| ATOM | 2487 | NZ | LYS | A | 266 | −20.288 | −11.924 | −0.440 | 1.00 | 10.00 | A | N |
| ATOM | 2491 | C | LYS | A | 266 | −17.475 | −17.735 | 0.427 | 1.00 | 10.00 | A | C |
| ATOM | 2492 | O | LYS | A | 266 | −17.236 | −17.164 | −0.639 | 1.00 | 10.00 | A | O |
| ATOM | 2493 | N | ASN | A | 267 | −17.013 | −18.939 | 0.726 | 1.00 | 10.00 | A | N |
| ATOM | 2495 | CA | ASN | A | 267 | −16.183 | −19.700 | −0.196 | 1.00 | 10.00 | A | C |
| ATOM | 2496 | CB | ASN | A | 267 | −15.548 | −20.901 | 0.520 | 1.00 | 10.00 | A | C |
| ATOM | 2497 | CG | ASN | A | 267 | −16.563 | −21.820 | 1.174 | 1.00 | 10.00 | A | C |
| ATOM | 2498 | OD1 | ASN | A | 267 | −17.056 | −22.767 | 0.559 | 1.00 | 10.00 | A | O |
| ATOM | 2499 | ND2 | ASN | A | 267 | −16.867 | −21.554 | 2.435 | 1.00 | 10.00 | A | N |
| ATOM | 2502 | C | ASN | A | 267 | −16.995 | −20.165 | −1.400 | 1.00 | 10.00 | A | C |
| ATOM | 2503 | O | ASN | A | 267 | −18.205 | −20.366 | −1.287 | 1.00 | 10.00 | A | O |
| ATOM | 2504 | N | LEU | A | 268 | −16.324 | −20.301 | −2.549 | 1.00 | 10.00 | A | N |
| ATOM | 2506 | CA | LEU | A | 268 | −16.952 | −20.739 | −3.802 | 1.00 | 10.00 | A | C |
| ATOM | 2507 | CB | LEU | A | 268 | −17.741 | −22.048 | −3.640 | 1.00 | 10.00 | A | C |
| ATOM | 2508 | CG | LEU | A | 268 | −16.922 | −23.300 | −3.309 | 1.00 | 10.00 | A | C |
| ATOM | 2509 | CD1 | LEU | A | 268 | −17.843 | −24.483 | −3.057 | 1.00 | 10.00 | A | C |
| ATOM | 2510 | CD2 | LEU | A | 268 | −15.947 | −23.617 | −4.430 | 1.00 | 10.00 | A | C |
| ATOM | 2511 | C | LEU | A | 268 | −17.809 | −19.642 | −4.440 | 1.00 | 10.00 | A | C |
| ATOM | 2512 | O | LEU | A | 268 | −17.839 | −19.495 | −5.662 | 1.00 | 10.00 | A | O |
| ATOM | 2513 | N | ALA | A | 269 | −18.487 | −18.864 | −3.611 | 1.00 | 10.00 | A | N |
| ATOM | 2515 | CA | ALA | A | 269 | −19.339 | −17.784 | −4.090 | 1.00 | 10.00 | A | C |
| ATOM | 2516 | CB | ALA | A | 269 | −20.684 | −17.824 | −3.381 | 1.00 | 10.00 | A | C |
| ATOM | 2517 | C | ALA | A | 269 | −18.664 | −16.430 | −3.888 | 1.00 | 10.00 | A | C |
| ATOM | 2518 | O | ALA | A | 269 | −19.309 | −15.380 | −3.946 | 1.00 | 10.00 | A | O |
| ATOM | 2519 | N | PHE | A | 270 | −17.359 | −16.468 | −3.659 | 1.00 | 10.00 | A | N |
| ATOM | 2521 | CA | PHE | A | 270 | −16.578 | −15.256 | −3.441 | 1.00 | 10.00 | A | C |
| ATOM | 2522 | CB | PHE | A | 270 | −15.253 | −15.588 | −2.741 | 1.00 | 10.00 | A | C |
| ATOM | 2523 | CG | PHE | A | 270 | −14.468 | −14.380 | −2.306 | 1.00 | 10.00 | A | C |
| ATOM | 2524 | CD1 | PHE | A | 270 | −14.953 | −13.541 | −1.316 | 1.00 | 10.00 | A | C |
| ATOM | 2525 | CD2 | PHE | A | 270 | −13.245 | −14.081 | −2.892 | 1.00 | 10.00 | A | C |
| ATOM | 2526 | CE1 | PHE | A | 270 | −14.238 | −12.427 | −0.917 | 1.00 | 10.00 | A | C |
| ATOM | 2527 | CE2 | PHE | A | 270 | −12.526 | −12.968 | −2.495 | 1.00 | 10.00 | A | C |
| ATOM | 2528 | CZ | PHE | A | 270 | −13.021 | −12.140 | −1.507 | 1.00 | 10.00 | A | C |
| ATOM | 2529 | C | PHE | A | 270 | −16.309 | −14.547 | −4.764 | 1.00 | 10.00 | A | C |
| ATOM | 2530 | O | PHE | A | 270 | −16.196 | −15.186 | −5.813 | 1.00 | 10.00 | A | O |
| ATOM | 2531 | N | ASP | A | 271 | −16.217 | −13.226 | −4.711 | 1.00 | 10.00 | A | N |
| ATOM | 2533 | CA | ASP | A | 271 | −15.958 | −12.429 | −5.901 | 1.00 | 10.00 | A | C |
| ATOM | 2534 | CB | ASP | A | 271 | −17.256 | −11.820 | −6.427 | 1.00 | 10.00 | A | C |
| ATOM | 2535 | CG | ASP | A | 271 | −17.141 | −11.387 | −7.873 | 1.00 | 10.00 | A | C |
| ATOM | 2536 | OD1 | ASP | A | 271 | −17.820 | −11.988 | −8.732 | 1.00 | 10.00 | A | O |
| ATOM | 2537 | OD2 | ASP | A | 271 | −16.370 | −10.451 | −8.154 | 1.00 | 10.00 | A | O |
| ATOM | 2538 | C | ASP | A | 271 | −14.955 | −11.331 | −5.575 | 1.00 | 10.00 | A | C |
| ATOM | 2539 | O | ASP | A | 271 | −15.145 | −10.577 | −4.625 | 1.00 | 10.00 | A | O |
| ATOM | 2540 | N | GLU | A | 272 | −13.896 | −11.240 | −6.362 | 1.00 | 10.00 | A | N |
| ATOM | 2542 | CA | GLU | A | 272 | −12.854 | −10.242 | −6.134 | 1.00 | 10.00 | A | C |
| ATOM | 2543 | CB | GLU | A | 272 | −11.471 | −10.809 | −6.467 | 1.00 | 10.00 | A | C |
| ATOM | 2544 | CG | GLU | A | 272 | −11.486 | −12.112 | −7.256 | 1.00 | 10.00 | A | C |
| ATOM | 2545 | CD | GLU | A | 272 | −12.035 | −11.953 | −8.660 | 1.00 | 10.00 | A | C |
| ATOM | 2546 | OE1 | GLU | A | 272 | −11.245 | −11.695 | −9.586 | 1.00 | 10.00 | A | O |
| ATOM | 2547 | OE2 | GLU | A | 272 | −13.262 | −12.098 | −8.840 | 1.00 | 10.00 | A | O |
| ATOM | 2548 | C | GLU | A | 272 | −13.110 | −8.960 | −6.925 | 1.00 | 10.00 | A | C |
| ATOM | 2549 | O | GLU | A | 272 | −12.407 | −7.963 | −6.763 | 1.00 | 10.00 | A | O |
| ATOM | 2550 | N | LEU | A | 273 | −14.124 | −8.987 | −7.766 | 1.00 | 10.00 | A | N |
| ATOM | 2552 | CA | LEU | A | 273 | −14.468 | −7.830 | −8.575 | 1.00 | 10.00 | A | C |
| ATOM | 2553 | CB | LEU | A | 273 | −14.741 | −8.257 | −10.022 | 1.00 | 10.00 | A | C |
| ATOM | 2554 | CG | LEU | A | 273 | −15.167 | −7.155 | −10.995 | 1.00 | 10.00 | A | C |
| ATOM | 2555 | CD1 | LEU | A | 273 | −14.073 | −6.110 | −11.149 | 1.00 | 10.00 | A | C |
| ATOM | 2556 | CD2 | LEU | A | 273 | −15.534 | −7.753 | −12.342 | 1.00 | 10.00 | A | C |
| ATOM | 2557 | C | LEU | A | 273 | −15.676 | −7.105 | −7.998 | 1.00 | 10.00 | A | C |
| ATOM | 2558 | O | LEU | A | 273 | −15.845 | −5.903 | −8.195 | 1.00 | 10.00 | A | O |
| ATOM | 2559 | N | PHE | A | 274 | −16.513 | −7.839 | −7.280 | 1.00 | 10.00 | A | N |
| ATOM | 2561 | CA | PHE | A | 274 | −17.712 | −7.260 | −6.685 | 1.00 | 10.00 | A | C |
| ATOM | 2562 | CB | PHE | A | 274 | −18.936 | −8.148 | −6.932 | 1.00 | 10.00 | A | C |
| ATOM | 2563 | CG | PHE | A | 274 | −19.231 | −8.439 | −8.379 | 1.00 | 10.00 | A | C |
| ATOM | 2564 | CD1 | PHE | A | 274 | −18.746 | −7.620 | −9.386 | 1.00 | 10.00 | A | C |
| ATOM | 2565 | CD2 | PHE | A | 274 | −19.996 | −9.538 | −8.730 | 1.00 | 10.00 | A | C |
| ATOM | 2566 | CE1 | PHE | A | 274 | −19.020 | −7.892 | −10.712 | 1.00 | 10.00 | A | C |
| ATOM | 2567 | CE2 | PHE | A | 274 | −20.272 | −9.817 | −10.054 | 1.00 | 10.00 | A | C |
| ATOM | 2568 | CZ | PHE | A | 274 | −19.784 | −8.993 | −11.046 | 1.00 | 10.00 | A | C |
| ATOM | 2569 | C | PHE | A | 274 | −17.569 | −6.997 | −5.187 | 1.00 | 10.00 | A | C |
| ATOM | 2570 | O | PHE | A | 274 | −17.751 | −5.866 | −4.734 | 1.00 | 10.00 | A | O |
| ATOM | 2571 | N | ASP | A | 275 | −17.240 | −8.040 | −4.422 | 1.00 | 10.00 | A | N |
| ATOM | 2573 | CA | ASP | A | 275 | −17.123 | −7.925 | −2.962 | 1.00 | 10.00 | A | C |
| ATOM | 2574 | CB | ASP | A | 275 | −16.934 | −9.296 | −2.301 | 1.00 | 10.00 | A | C |
| ATOM | 2575 | CG | ASP | A | 275 | −17.530 | −9.368 | −0.904 | 1.00 | 10.00 | A | C |
| ATOM | 2576 | OD1 | ASP | A | 275 | −16.869 | −8.940 | 0.066 | 1.00 | 10.00 | A | O |
| ATOM | 2577 | OD2 | ASP | A | 275 | −18.671 | −9.863 | −0.769 | 1.00 | 10.00 | A | O |
| ATOM | 2578 | C | ASP | A | 275 | −16.021 | −6.965 | −2.517 | 1.00 | 10.00 | A | C |
| ATOM | 2579 | O | ASP | A | 275 | −16.177 | −6.249 | −1.527 | 1.00 | 10.00 | A | O |
| ATOM | 2580 | N | VAL | A | 276 | −14.921 | −6.927 | −3.254 | 1.00 | 10.00 | A | N |
| ATOM | 2582 | CA | VAL | A | 276 | −13.796 | −6.055 | −2.909 | 1.00 | 10.00 | A | C |
| ATOM | 2583 | CB | VAL | A | 276 | −12.448 | −6.593 | −3.448 | 1.00 | 10.00 | A | C |
| ATOM | 2584 | CG1 | VAL | A | 276 | −11.277 | −5.991 | −2.679 | 1.00 | 10.00 | A | C |
| ATOM | 2585 | CG2 | VAL | A | 276 | −12.406 | −8.111 | −3.354 | 1.00 | 10.00 | A | C |
| ATOM | 2586 | C | VAL | A | 276 | −14.031 | −4.609 | −3.369 | 1.00 | 10.00 | A | C |
| ATOM | 2587 | O | VAL | A | 276 | −13.101 | −3.892 | −3.732 | 1.00 | 10.00 | A | O |
| ATOM | 2588 | N | ARG | A | 277 | −15.287 | −4.188 | −3.350 | 1.00 | 10.00 | A | N |
| ATOM | 2590 | CA | ARG | A | 277 | −15.649 | −2.837 | −3.737 | 1.00 | 10.00 | A | C |
| ATOM | 2591 | CB | ARG | A | 277 | −16.498 | −2.838 | −5.007 | 1.00 | 10.00 | A | C |
| ATOM | 2592 | CG | ARG | A | 277 | −15.777 | −3.322 | −6.252 | 1.00 | 10.00 | A | C |
| ATOM | 2593 | CD | ARG | A | 277 | −16.607 | −3.054 | −7.496 | 1.00 | 10.00 | A | C |
| ATOM | 2594 | NE | ARG | A | 277 | −17.965 | −3.588 | −7.373 | 1.00 | 10.00 | A | N |
| ATOM | 2596 | CZ | ARG | A | 277 | −18.753 | −3.854 | −8.411 | 1.00 | 10.00 | A | C |
| ATOM | 2597 | NH1 | ARG | A | 277 | −18.320 | −3.646 | −9.647 | 1.00 | 10.00 | A | N |
| ATOM | 2600 | NH2 | ARG | A | 277 | −19.979 | −4.317 | −8.212 | 1.00 | 10.00 | A | N |
| ATOM | 2603 | C | ARG | A | 277 | −16.430 | −2.172 | −2.615 | 1.00 | 10.00 | A | C |
| ATOM | 2604 | O | ARG | A | 277 | −17.509 | −2.628 | −2.249 | 1.00 | 10.00 | A | O |
| ATOM | 2605 | N | ALA | A | 278 | −15.883 | −1.103 | −2.070 | 1.00 | 10.00 | A | N |
| ATOM | 2607 | CA | ALA | A | 278 | −16.536 | −0.389 | −0.991 | 1.00 | 10.00 | A | C |
| ATOM | 2608 | CB | ALA | A | 278 | −15.762 | −0.563 | 0.307 | 1.00 | 10.00 | A | C |
| ATOM | 2609 | C | ALA | A | 278 | −16.699 | 1.084 | −1.341 | 1.00 | 10.00 | A | C |
| ATOM | 2610 | O | ALA | A | 278 | −15.731 | 1.850 | −1.322 | 1.00 | 10.00 | A | O |
| ATOM | 2611 | N | VAL | A | 279 | −17.922 | 1.467 | −1.678 | 1.00 | 10.00 | A | N |
| ATOM | 2613 | CA | VAL | A | 279 | −18.229 | 2.844 | −2.033 | 1.00 | 10.00 | A | C |
| ATOM | 2614 | CB | VAL | A | 279 | −19.404 | 2.915 | −3.032 | 1.00 | 10.00 | A | C |
| ATOM | 2615 | CG1 | VAL | A | 279 | −19.688 | 4.359 | −3.430 | 1.00 | 10.00 | A | C |
| ATOM | 2616 | CG2 | VAL | A | 279 | −19.109 | 2.065 | −4.263 | 1.00 | 10.00 | A | C |
| ATOM | 2617 | C | VAL | A | 279 | −18.588 | 3.619 | −0.772 | 1.00 | 10.00 | A | C |
| ATOM | 2618 | O | VAL | A | 279 | −19.671 | 3.451 | −0.220 | 1.00 | 10.00 | A | O |
| ATOM | 2619 | N | ARG | A | 280 | −17.679 | 4.458 | −0.316 | 1.00 | 10.00 | A | N |
| ATOM | 2621 | CA | ARG | A | 280 | −17.901 | 5.232 | 0.891 | 1.00 | 10.00 | A | C |
| ATOM | 2622 | CB | ARG | A | 280 | −16.600 | 5.349 | 1.683 | 1.00 | 10.00 | A | C |
| ATOM | 2623 | CG | ARG | A | 280 | −16.732 | 6.103 | 2.992 | 1.00 | 10.00 | A | C |
| ATOM | 2624 | CD | ARG | A | 280 | −15.619 | 5.722 | 3.949 | 1.00 | 10.00 | A | C |
| ATOM | 2625 | NE | ARG | A | 280 | −15.742 | 4.339 | 4.400 | 1.00 | 10.00 | A | N |
| ATOM | 2627 | CZ | ARG | A | 280 | −15.075 | 3.829 | 5.429 | 1.00 | 10.00 | A | C |
| ATOM | 2628 | NH1 | ARG | A | 280 | −14.228 | 4.579 | 6.125 | 1.00 | 10.00 | A | N |
| ATOM | 2631 | NH2 | ARG | A | 280 | −15.266 | 2.558 | 5.760 | 1.00 | 10.00 | A | N |
| ATOM | 2634 | C | ARG | A | 280 | −18.469 | 6.611 | 0.587 | 1.00 | 10.00 | A | C |
| ATOM | 2635 | O | ARG | A | 280 | −17.941 | 7.343 | −0.251 | 1.00 | 10.00 | A | O |
| ATOM | 2636 | N | ILE | A | 281 | −19.549 | 6.948 | 1.277 | 1.00 | 10.00 | A | N |
| ATOM | 2638 | CA | ILE | A | 281 | −20.202 | 8.234 | 1.121 | 1.00 | 10.00 | A | C |
| ATOM | 2639 | CB | ILE | A | 281 | −21.664 | 8.080 | 0.645 | 1.00 | 10.00 | A | C |
| ATOM | 2640 | CG1 | ILE | A | 281 | −21.722 | 7.371 | −0.715 | 1.00 | 10.00 | A | C |
| ATOM | 2641 | CG2 | ILE | A | 281 | −22.351 | 9.441 | 0.574 | 1.00 | 10.00 | A | C |
| ATOM | 2642 | CD1 | ILE | A | 281 | −23.122 | 7.193 | −1.258 | 1.00 | 10.00 | A | C |
| ATOM | 2643 | C | ILE | A | 281 | −20.194 | 8.963 | 2.455 | 1.00 | 10.00 | A | C |
| ATOM | 2644 | O | ILE | A | 281 | −20.941 | 8.616 | 3.373 | 1.00 | 10.00 | A | O |
| ATOM | 2645 | N | VAL | A | 282 | −19.324 | 9.951 | 2.565 | 1.00 | 10.00 | A | N |
| ATOM | 2647 | CA | VAL | A | 282 | −19.203 | 10.734 | 3.786 | 1.00 | 10.00 | A | C |
| ATOM | 2648 | CB | VAL | A | 282 | −17.735 | 11.051 | 4.126 | 1.00 | 10.00 | A | C |
| ATOM | 2649 | CG1 | VAL | A | 282 | −17.629 | 11.592 | 5.542 | 1.00 | 10.00 | A | C |
| ATOM | 2650 | CG2 | VAL | A | 282 | −16.858 | 9.818 | 3.956 | 1.00 | 10.00 | A | C |
| ATOM | 2651 | C | VAL | A | 282 | −19.989 | 12.037 | 3.673 | 1.00 | 10.00 | A | C |
| ATOM | 2652 | O | VAL | A | 282 | −19.513 | 13.019 | 3.097 | 1.00 | 10.00 | A | O |
| ATOM | 2653 | N | ALA | A | 283 | −21.188 | 12.038 | 4.231 | 1.00 | 10.00 | A | N |
| ATOM | 2655 | CA | ALA | A | 283 | −22.059 | 13.203 | 4.193 | 1.00 | 10.00 | A | C |
| ATOM | 2656 | CB | ALA | A | 283 | −23.500 | 12.787 | 4.437 | 1.00 | 10.00 | A | C |
| ATOM | 2657 | C | ALA | A | 283 | −21.624 | 14.249 | 5.213 | 1.00 | 10.00 | A | C |
| ATOM | 2658 | O | ALA | A | 283 | −20.569 | 14.123 | 5.840 | 1.00 | 10.00 | A | O |
| ATOM | 2659 | N | GLU | A | 284 | −22.427 | 15.290 | 5.371 | 1.00 | 10.00 | A | N |
| ATOM | 2661 | CA | GLU | A | 284 | −22.110 | 16.344 | 6.318 | 1.00 | 10.00 | A | C |
| ATOM | 2662 | CB | GLU | A | 284 | −22.297 | 17.720 | 5.677 | 1.00 | 10.00 | A | C |
| ATOM | 2663 | CG | GLU | A | 284 | −21.464 | 18.811 | 6.325 | 1.00 | 10.00 | A | C |
| ATOM | 2664 | CD | GLU | A | 284 | −20.021 | 18.389 | 6.511 | 1.00 | 10.00 | A | C |
| ATOM | 2665 | OE1 | GLU | A | 284 | −19.329 | 18.167 | 5.494 | 1.00 | 10.00 | A | O |
| ATOM | 2666 | OE2 | GLU | A | 284 | −19.578 | 18.270 | 7.671 | 1.00 | 10.00 | A | O |
| ATOM | 2667 | C | GLU | A | 284 | −22.966 | 16.210 | 7.570 | 1.00 | 10.00 | A | C |
| ATOM | 2668 | O | GLU | A | 284 | −22.453 | 16.007 | 8.670 | 1.00 | 10.00 | A | O |
| ATOM | 2669 | N | ARG | A | 285 | −24.272 | 16.313 | 7.395 | 1.00 | 10.00 | A | N |
| ATOM | 2671 | CA | ARG | A | 285 | −25.203 | 16.197 | 8.502 | 1.00 | 10.00 | A | C |
| ATOM | 2672 | CB | ARG | A | 285 | −26.177 | 17.375 | 8.498 | 1.00 | 10.00 | A | C |
| ATOM | 2673 | CG | ARG | A | 285 | −25.505 | 18.732 | 8.363 | 1.00 | 10.00 | A | C |
| ATOM | 2674 | CD | ARG | A | 285 | −26.501 | 19.862 | 8.541 | 1.00 | 10.00 | A | C |
| ATOM | 2675 | NE | ARG | A | 285 | −26.958 | 19.976 | 9.925 | 1.00 | 10.00 | A | N |
| ATOM | 2677 | CZ | ARG | A | 285 | −28.156 | 20.444 | 10.279 | 1.00 | 10.00 | A | C |
| ATOM | 2678 | NH1 | ARG | A | 285 | −29.017 | 20.840 | 9.351 | 1.00 | 10.00 | A | N |
| ATOM | 2681 | NH2 | ARG | A | 285 | −28.489 | 20.512 | 11.565 | 1.00 | 10.00 | A | N |
| ATOM | 2684 | C | ARG | A | 285 | −25.961 | 14.879 | 8.417 | 1.00 | 10.00 | A | C |
| ATOM | 2685 | O | ARG | A | 285 | −25.893 | 14.179 | 7.400 | 1.00 | 10.00 | A | O |
| ATOM | 2686 | N | LEU | A | 286 | −26.681 | 14.541 | 9.479 | 1.00 | 10.00 | A | N |
| ATOM | 2688 | CA | LEU | A | 286 | −27.444 | 13.298 | 9.520 | 1.00 | 10.00 | A | C |
| ATOM | 2689 | CB | LEU | A | 286 | −28.076 | 13.094 | 10.898 | 1.00 | 10.00 | A | C |
| ATOM | 2690 | CG | LEU | A | 286 | −27.103 | 12.961 | 12.068 | 1.00 | 10.00 | A | C |
| ATOM | 2691 | CD1 | LEU | A | 286 | −27.857 | 12.970 | 13.386 | 1.00 | 10.00 | A | C |
| ATOM | 2692 | CD2 | LEU | A | 286 | −26.273 | 11.695 | 11.936 | 1.00 | 10.00 | A | C |
| ATOM | 2693 | C | LEU | A | 286 | −28.525 | 13.310 | 8.452 | 1.00 | 10.00 | A | C |
| ATOM | 2694 | O | LEU | A | 286 | −28.792 | 12.295 | 7.805 | 1.00 | 10.00 | A | O |
| ATOM | 2695 | N | GLN | A | 287 | −29.122 | 14.481 | 8.259 | 1.00 | 10.00 | A | N |
| ATOM | 2697 | CA | GLN | A | 287 | −30.177 | 14.659 | 7.267 | 1.00 | 10.00 | A | C |
| ATOM | 2698 | CB | GLN | A | 287 | −30.808 | 16.044 | 7.409 | 1.00 | 10.00 | A | C |
| ATOM | 2699 | CG | GLN | A | 287 | −31.289 | 16.340 | 8.821 | 1.00 | 10.00 | A | C |
| ATOM | 2700 | CD | GLN | A | 287 | −31.785 | 17.758 | 8.996 | 1.00 | 10.00 | A | C |
| ATOM | 2701 | OE1 | GLN | A | 287 | −31.334 | 18.679 | 8.317 | 1.00 | 10.00 | A | O |
| ATOM | 2702 | NE2 | GLN | A | 287 | −32.711 | 17.942 | 9.917 | 1.00 | 10.00 | A | N |
| ATOM | 2705 | C | GLN | A | 287 | −29.634 | 14.441 | 5.854 | 1.00 | 10.00 | A | C |
| ATOM | 2706 | O | GLN | A | 287 | −30.361 | 14.016 | 4.955 | 1.00 | 10.00 | A | O |
| ATOM | 2707 | N | ASP | A | 288 | −28.343 | 14.714 | 5.673 | 1.00 | 10.00 | A | N |
| ATOM | 2709 | CA | ASP | A | 288 | −27.693 | 14.535 | 4.373 | 1.00 | 10.00 | A | C |
| ATOM | 2710 | CB | ASP | A | 288 | −26.322 | 15.214 | 4.335 | 1.00 | 10.00 | A | C |
| ATOM | 2711 | CG | ASP | A | 288 | −26.387 | 16.719 | 4.423 | 1.00 | 10.00 | A | C |
| ATOM | 2712 | OD1 | ASP | A | 288 | −27.182 | 17.337 | 3.682 | 1.00 | 10.00 | A | O |
| ATOM | 2713 | OD2 | ASP | A | 288 | −25.634 | 17.295 | 5.232 | 1.00 | 10.00 | A | O |
| ATOM | 2714 | C | ASP | A | 288 | −27.518 | 13.055 | 4.085 | 1.00 | 10.00 | A | C |
| ATOM | 2715 | O | ASP | A | 288 | −27.616 | 12.612 | 2.938 | 1.00 | 10.00 | A | O |
| ATOM | 2716 | N | CYS | A | 289 | −27.268 | 12.290 | 5.142 | 1.00 | 10.00 | A | N |
| ATOM | 2718 | CA | CYS | A | 289 | −27.079 | 10.852 | 5.024 | 1.00 | 10.00 | A | C |
| ATOM | 2719 | CB | CYS | A | 289 | −26.650 | 10.259 | 6.369 | 1.00 | 10.00 | A | C |
| ATOM | 2720 | SG | CYS | A | 289 | −25.271 | 11.114 | 7.164 | 1.00 | 10.00 | A | S |
| ATOM | 2722 | C | CYS | A | 289 | −28.361 | 10.181 | 4.544 | 1.00 | 10.00 | A | C |
| ATOM | 2723 | O | CYS | A | 289 | −28.330 | 9.263 | 3.716 | 1.00 | 10.00 | A | O |
| ATOM | 2724 | N | TYR | A | 290 | −29.491 | 10.652 | 5.062 | 1.00 | 10.00 | A | N |
| ATOM | 2726 | CA | TYR | A | 290 | −30.787 | 10.108 | 4.678 | 1.00 | 10.00 | A | C |
| ATOM | 2727 | CB | TYR | A | 290 | −31.904 | 10.618 | 5.600 | 1.00 | 10.00 | A | C |
| ATOM | 2728 | CG | TYR | A | 290 | −31.800 | 10.133 | 7.030 | 1.00 | 10.00 | A | C |
| ATOM | 2729 | CD1 | TYR | A | 290 | −31.823 | 8.777 | 7.332 | 1.00 | 10.00 | A | C |
| ATOM | 2730 | CD2 | TYR | A | 290 | −31.674 | 11.035 | 8.076 | 1.00 | 10.00 | A | C |
| ATOM | 2731 | CE1 | TYR | A | 290 | −31.721 | 8.333 | 8.637 | 1.00 | 10.00 | A | C |
| ATOM | 2732 | CE2 | TYR | A | 290 | −31.573 | 10.599 | 9.386 | 1.00 | 10.00 | A | C |
| ATOM | 2733 | CZ | TYR | A | 290 | −31.596 | 9.249 | 9.662 | 1.00 | 10.00 | A | C |
| ATOM | 2734 | OH | TYR | A | 290 | −31.495 | 8.812 | 10.968 | 1.00 | 10.00 | A | O |
| ATOM | 2736 | C | TYR | A | 290 | −31.095 | 10.452 | 3.227 | 1.00 | 10.00 | A | C |
| ATOM | 2737 | O | TYR | A | 290 | −31.620 | 9.622 | 2.480 | 1.00 | 10.00 | A | O |
| ATOM | 2738 | N | ALA | A | 291 | −30.758 | 11.677 | 2.832 | 1.00 | 10.00 | A | N |
| ATOM | 2740 | CA | ALA | A | 291 | −30.984 | 12.127 | 1.466 | 1.00 | 10.00 | A | C |
| ATOM | 2741 | CB | ALA | A | 291 | −30.669 | 13.611 | 1.338 | 1.00 | 10.00 | A | C |
| ATOM | 2742 | C | ALA | A | 291 | −30.124 | 11.312 | 0.508 | 1.00 | 10.00 | A | C |
| ATOM | 2743 | O | ALA | A | 291 | −30.570 | 10.926 | −0.571 | 1.00 | 10.00 | A | O |
| ATOM | 2744 | N | ALA | A | 292 | −28.888 | 11.041 | 0.927 | 1.00 | 10.00 | A | N |
| ATOM | 2746 | CA | ALA | A | 292 | −27.955 | 10.256 | 0.122 | 1.00 | 10.00 | A | C |
| ATOM | 2747 | CB | ALA | A | 292 | −26.597 | 10.181 | 0.806 | 1.00 | 10.00 | A | C |
| ATOM | 2748 | C | ALA | A | 292 | −28.506 | 8.854 | −0.104 | 1.00 | 10.00 | A | C |
| ATOM | 2749 | O | ALA | A | 292 | −28.528 | 8.355 | −1.230 | 1.00 | 10.00 | A | O |
| ATOM | 2750 | N | LEU | A | 293 | −28.955 | 8.233 | 0.982 | 1.00 | 10.00 | A | N |
| ATOM | 2752 | CA | LEU | A | 293 | −29.520 | 6.892 | 0.928 | 1.00 | 10.00 | A | C |
| ATOM | 2753 | CB | LEU | A | 293 | −29.963 | 6.456 | 2.327 | 1.00 | 10.00 | A | C |
| ATOM | 2754 | CG | LEU | A | 293 | −30.533 | 5.043 | 2.440 | 1.00 | 10.00 | A | C |
| ATOM | 2755 | CD1 | LEU | A | 293 | −29.447 | 4.007 | 2.182 | 1.00 | 10.00 | A | C |
| ATOM | 2756 | CD2 | LEU | A | 293 | −31.173 | 4.835 | 3.803 | 1.00 | 10.00 | A | C |
| ATOM | 2757 | C | LEU | A | 293 | −30.707 | 6.846 | −0.028 | 1.00 | 10.00 | A | C |
| ATOM | 2758 | O | LEU | A | 293 | −30.901 | 5.871 | −0.748 | 1.00 | 10.00 | A | O |
| ATOM | 2759 | N | GLY | A | 294 | −31.488 | 7.916 | −0.033 | 1.00 | 10.00 | A | N |
| ATOM | 2761 | CA | GLY | A | 294 | −32.649 | 7.991 | −0.894 | 1.00 | 10.00 | A | C |
| ATOM | 2762 | C | GLY | A | 294 | −32.286 | 8.133 | −2.359 | 1.00 | 10.00 | A | C |
| ATOM | 2763 | O | GLY | A | 294 | −32.763 | 7.365 | −3.201 | 1.00 | 10.00 | A | O |
| ATOM | 2764 | N | ILE | A | 295 | −31.423 | 9.094 | −2.671 | 1.00 | 10.00 | A | N |
| ATOM | 2766 | CA | ILE | A | 295 | −31.021 | 9.329 | −4.052 | 1.00 | 10.00 | A | C |
| ATOM | 2767 | CB | ILE | A | 295 | −30.219 | 10.634 | −4.242 | 1.00 | 10.00 | A | C |
| ATOM | 2768 | CG1 | ILE | A | 295 | −28.940 | 10.626 | −3.402 | 1.00 | 10.00 | A | C |
| ATOM | 2769 | CG2 | ILE | A | 295 | −31.084 | 11.839 | −3.900 | 1.00 | 10.00 | A | C |
| ATOM | 2770 | CD1 | ILE | A | 295 | −27.985 | 11.754 | −3.727 | 1.00 | 10.00 | A | C |
| ATOM | 2771 | C | ILE | A | 295 | −30.281 | 8.139 | −4.665 | 1.00 | 10.00 | A | C |
| ATOM | 2772 | O | ILE | A | 295 | −30.499 | 7.807 | −5.832 | 1.00 | 10.00 | A | O |
| ATOM | 2773 | N | VAL | A | 296 | −29.425 | 7.477 | −3.887 | 1.00 | 10.00 | A | N |
| ATOM | 2775 | CA | VAL | A | 296 | −28.684 | 6.326 | −4.405 | 1.00 | 10.00 | A | C |
| ATOM | 2776 | CB | VAL | A | 296 | −27.503 | 5.883 | −3.512 | 1.00 | 10.00 | A | C |
| ATOM | 2777 | CG1 | VAL | A | 296 | −26.502 | 7.016 | −3.340 | 1.00 | 10.00 | A | C |
| ATOM | 2778 | CG2 | VAL | A | 296 | −27.980 | 5.367 | −2.162 | 1.00 | 10.00 | A | C |
| ATOM | 2779 | C | VAL | A | 296 | −29.609 | 5.149 | −4.712 | 1.00 | 10.00 | A | C |
| ATOM | 2780 | O | VAL | A | 296 | −29.408 | 4.432 | −5.696 | 1.00 | 10.00 | A | O |
| ATOM | 2781 | N | HIS | A | 297 | −30.639 | 4.979 | −3.888 | 1.00 | 10.00 | A | N |
| ATOM | 2783 | CA | HIS | A | 297 | −31.604 | 3.900 | −4.074 | 1.00 | 10.00 | A | C |
| ATOM | 2784 | CB | HIS | A | 297 | −32.480 | 3.739 | −2.826 | 1.00 | 10.00 | A | C |
| ATOM | 2785 | CG | HIS | A | 297 | −31.891 | 2.859 | −1.764 | 1.00 | 10.00 | A | C |
| ATOM | 2786 | ND1 | HIS | A | 297 | −32.385 | 2.765 | −0.481 | 1.00 | 10.00 | A | N |
| ATOM | 2788 | CD2 | HIS | A | 297 | −30.826 | 2.022 | −1.813 | 1.00 | 10.00 | A | C |
| ATOM | 2789 | CE1 | HIS | A | 297 | −31.629 | 1.887 | 0.192 | 1.00 | 10.00 | A | C |
| ATOM | 2790 | NE2 | HIS | A | 297 | −30.666 | 1.407 | −0.572 | 1.00 | 10.00 | A | N |
| ATOM | 2791 | C | HIS | A | 297 | −32.480 | 4.177 | −5.289 | 1.00 | 10.00 | A | C |
| ATOM | 2792 | O | HIS | A | 297 | −33.037 | 3.264 | −5.896 | 1.00 | 10.00 | A | O |
| ATOM | 2793 | N | THR | A | 298 | −32.588 | 5.448 | −5.643 | 1.00 | 10.00 | A | N |
| ATOM | 2795 | CA | THR | A | 298 | −33.388 | 5.861 | −6.781 | 1.00 | 10.00 | A | C |
| ATOM | 2796 | CB | THR | A | 298 | −33.853 | 7.320 | −6.618 | 1.00 | 10.00 | A | C |
| ATOM | 2797 | OG1 | THR | A | 298 | −34.445 | 7.477 | −5.317 | 1.00 | 10.00 | A | O |
| ATOM | 2799 | CG2 | THR | A | 298 | −34.887 | 7.677 | −7.679 | 1.00 | 10.00 | A | C |
| ATOM | 2800 | C | THR | A | 298 | −32.597 | 5.697 | −8.075 | 1.00 | 10.00 | A | C |
| ATOM | 2801 | O | THR | A | 298 | −33.168 | 5.518 | −9.154 | 1.00 | 10.00 | A | O |
| ATOM | 2802 | N | HIS | A | 299 | −31.276 | 5.732 | −7.955 | 1.00 | 10.00 | A | N |
| ATOM | 2804 | CA | HIS | A | 299 | −30.401 | 5.581 | −9.108 | 1.00 | 10.00 | A | C |
| ATOM | 2805 | CB | HIS | A | 299 | −29.046 | 6.244 | −8.836 | 1.00 | 10.00 | A | C |
| ATOM | 2806 | CG | HIS | A | 299 | −28.231 | 6.536 | −10.065 | 1.00 | 10.00 | A | C |
| ATOM | 2807 | ND1 | HIS | A | 299 | −28.448 | 5.964 | −11.304 | 1.00 | 10.00 | A | N |
| ATOM | 2809 | CD2 | HIS | A | 299 | −27.173 | 7.370 | −10.226 | 1.00 | 10.00 | A | C |
| ATOM | 2810 | CE1 | HIS | A | 299 | −27.533 | 6.456 | −12.151 | 1.00 | 10.00 | A | C |
| ATOM | 2811 | NE2 | HIS | A | 299 | −26.737 | 7.315 | −11.546 | 1.00 | 10.00 | A | N |
| ATOM | 2812 | C | HIS | A | 299 | −30.205 | 4.102 | −9.427 | 1.00 | 10.00 | A | C |
| ATOM | 2813 | O | HIS | A | 299 | −30.293 | 3.686 | −10.585 | 1.00 | 10.00 | A | O |
| ATOM | 2814 | N | TYR | A | 300 | −29.952 | 3.316 | −8.392 | 1.00 | 10.00 | A | N |
| ATOM | 2816 | CA | TYR | A | 300 | −29.739 | 1.888 | −8.544 | 1.00 | 10.00 | A | C |
| ATOM | 2817 | CB | TYR | A | 300 | −28.259 | 1.546 | −8.343 | 1.00 | 10.00 | A | C |
| ATOM | 2818 | CG | TYR | A | 300 | −27.348 | 2.158 | −9.382 | 1.00 | 10.00 | A | C |
| ATOM | 2819 | CD1 | TYR | A | 300 | −27.438 | 1.773 | −10.711 | 1.00 | 10.00 | A | C |
| ATOM | 2820 | CD2 | TYR | A | 300 | −26.403 | 3.119 | −9.038 | 1.00 | 10.00 | A | C |
| ATOM | 2821 | CE1 | TYR | A | 300 | −26.618 | 2.328 | −11.672 | 1.00 | 10.00 | A | C |
| ATOM | 2822 | CE2 | TYR | A | 300 | −25.576 | 3.677 | −9.996 | 1.00 | 10.00 | A | C |
| ATOM | 2823 | CZ | TYR | A | 300 | −25.688 | 3.280 | −11.312 | 1.00 | 10.00 | A | C |
| ATOM | 2824 | OH | TYR | A | 300 | −24.878 | 3.837 | −12.274 | 1.00 | 10.00 | A | O |
| ATOM | 2826 | C | TYR | A | 300 | −30.597 | 1.125 | −7.549 | 1.00 | 10.00 | A | C |
| ATOM | 2827 | O | TYR | A | 300 | −30.711 | 1.517 | −6.391 | 1.00 | 10.00 | A | O |
| ATOM | 2828 | N | ARG | A | 301 | −31.217 | 0.052 | −8.014 | 1.00 | 10.00 | A | N |
| ATOM | 2830 | CA | ARG | A | 301 | −32.062 | −0.766 | −7.162 | 1.00 | 10.00 | A | C |
| ATOM | 2831 | CB | ARG | A | 301 | −32.942 | −1.699 | −7.992 | 1.00 | 10.00 | A | C |
| ATOM | 2832 | CG | ARG | A | 301 | −34.353 | −1.175 | −8.212 | 1.00 | 10.00 | A | C |
| ATOM | 2833 | CD | ARG | A | 301 | −35.363 | −2.306 | −8.257 | 1.00 | 10.00 | A | C |
| ATOM | 2834 | NE | ARG | A | 301 | −35.210 | −3.199 | −7.113 | 1.00 | 10.00 | A | N |
| ATOM | 2836 | CZ | ARG | A | 301 | −35.015 | −4.512 | −7.214 | 1.00 | 10.00 | A | C |
| ATOM | 2837 | NH1 | ARG | A | 301 | −34.955 | −5.090 | −8.412 | 1.00 | 10.00 | A | N |
| ATOM | 2840 | NH2 | ARG | A | 301 | −34.851 | −5.243 | −6.122 | 1.00 | 10.00 | A | N |
| ATOM | 2843 | C | ARG | A | 301 | −31.224 | −1.566 | −6.176 | 1.00 | 10.00 | A | C |
| ATOM | 2844 | O | ARG | A | 301 | −30.192 | −2.137 | −6.533 | 1.00 | 10.00 | A | O |
| ATOM | 2845 | N | HIS | A | 302 | −31.671 | −1.602 | −4.935 | 1.00 | 10.00 | A | N |
| ATOM | 2847 | CA | HIS | A | 302 | −30.960 | −2.325 | −3.892 | 1.00 | 10.00 | A | C |
| ATOM | 2848 | CB | HIS | A | 302 | −31.115 | −1.624 | −2.535 | 1.00 | 10.00 | A | C |
| ATOM | 2849 | CG | HIS | A | 302 | −32.526 | −1.567 | −2.026 | 1.00 | 10.00 | A | C |
| ATOM | 2850 | ND1 | HIS | A | 302 | −33.138 | −2.587 | −1.327 | 1.00 | 10.00 | A | N |
| ATOM | 2852 | CD2 | HIS | A | 302 | −33.443 | −0.569 | −2.106 | 1.00 | 10.00 | A | C |
| ATOM | 2853 | CE1 | HIS | A | 302 | −34.387 | −2.191 | −1.031 | 1.00 | 10.00 | A | C |
| ATOM | 2854 | NE2 | HIS | A | 302 | −34.620 | −0.968 | −1.477 | 1.00 | 10.00 | A | N |
| ATOM | 2855 | C | HIS | A | 302 | −31.432 | −3.766 | −3.791 | 1.00 | 10.00 | A | C |
| ATOM | 2856 | O | HIS | A | 302 | −32.511 | −4.118 | −4.270 | 1.00 | 10.00 | A | O |
| ATOM | 2857 | N | LEU | A | 303 | −30.621 | −4.592 | −3.154 | 1.00 | 10.00 | A | N |
| ATOM | 2859 | CA | LEU | A | 303 | −30.956 | −5.991 | −2.971 | 1.00 | 10.00 | A | C |
| ATOM | 2860 | CB | LEU | A | 303 | −29.694 | −6.857 | −2.874 | 1.00 | 10.00 | A | C |
| ATOM | 2861 | CG | LEU | A | 303 | −28.997 | −7.221 | −4.191 | 1.00 | 10.00 | A | C |
| ATOM | 2862 | CD1 | LEU | A | 303 | −30.007 | −7.659 | −5.243 | 1.00 | 10.00 | A | C |
| ATOM | 2863 | CD2 | LEU | A | 303 | −28.139 | −6.072 | −4.694 | 1.00 | 10.00 | A | C |
| ATOM | 2864 | C | LEU | A | 303 | −31.811 | −6.160 | −1.721 | 1.00 | 10.00 | A | C |
| ATOM | 2865 | O | LEU | A | 303 | −31.577 | −5.499 | −0.707 | 1.00 | 10.00 | A | O |
| ATOM | 2866 | N | PRO | A | 304 | −32.830 | −7.028 | −1.786 | 1.00 | 10.00 | A | N |
| ATOM | 2867 | CA | PRO | A | 304 | −33.724 | −7.283 | −0.656 | 1.00 | 10.00 | A | C |
| ATOM | 2868 | CB | PRO | A | 304 | −34.734 | −8.294 | −1.212 | 1.00 | 10.00 | A | C |
| ATOM | 2869 | CG | PRO | A | 304 | −34.632 | −8.164 | −2.692 | 1.00 | 10.00 | A | C |
| ATOM | 2870 | CD | PRO | A | 304 | −33.201 | −7.822 | −2.967 | 1.00 | 10.00 | A | C |
| ATOM | 2871 | C | PRO | A | 304 | −32.991 | −7.881 | 0.539 | 1.00 | 10.00 | A | C |
| ATOM | 2872 | O | PRO | A | 304 | −32.208 | −8.826 | 0.395 | 1.00 | 10.00 | A | O |
| ATOM | 2873 | N | ASP | A | 305 | −33.247 | −7.302 | 1.710 | 1.00 | 10.00 | A | N |
| ATOM | 2875 | CA | ASP | A | 305 | −32.650 | −7.743 | 2.974 | 1.00 | 10.00 | A | C |
| ATOM | 2876 | CB | ASP | A | 305 | −32.964 | −9.208 | 3.286 | 1.00 | 10.00 | A | C |
| ATOM | 2877 | CG | ASP | A | 305 | −34.326 | −9.388 | 3.916 | 1.00 | 10.00 | A | C |
| ATOM | 2878 | OD1 | ASP | A | 305 | −34.728 | −8.538 | 4.739 | 1.00 | 10.00 | A | O |
| ATOM | 2879 | OD2 | ASP | A | 305 | −35.002 | −10.387 | 3.596 | 1.00 | 10.00 | A | O |
| ATOM | 2880 | C | ASP | A | 305 | −31.154 | −7.469 | 3.060 | 1.00 | 10.00 | A | C |
| ATOM | 2881 | O | ASP | A | 305 | −30.457 | −8.056 | 3.886 | 1.00 | 10.00 | A | O |
| ATOM | 2882 | N | GLU | A | 306 | −30.670 | −6.559 | 2.225 | 1.00 | 10.00 | A | N |
| ATOM | 2884 | CA | GLU | A | 306 | −29.254 | −6.207 | 2.220 | 1.00 | 10.00 | A | C |
| ATOM | 2885 | CB | GLU | A | 306 | −28.657 | −6.353 | 0.824 | 1.00 | 10.00 | A | C |
| ATOM | 2886 | CG | GLU | A | 306 | −28.405 | −7.797 | 0.417 | 1.00 | 10.00 | A | C |
| ATOM | 2887 | CD | GLU | A | 306 | −27.281 | −8.453 | 1.199 | 1.00 | 10.00 | A | C |
| ATOM | 2888 | OE1 | GLU | A | 306 | −27.248 | −8.327 | 2.441 | 1.00 | 10.00 | A | O |
| ATOM | 2889 | OE2 | GLU | A | 306 | −26.428 | −9.117 | 0.569 | 1.00 | 10.00 | A | O |
| ATOM | 2890 | C | GLU | A | 306 | −29.019 | −4.811 | 2.787 | 1.00 | 10.00 | A | C |
| ATOM | 2891 | O | GLU | A | 306 | −28.052 | −4.128 | 2.438 | 1.00 | 10.00 | A | O |
| ATOM | 2892 | N | PHE | A | 307 | −29.913 | −4.399 | 3.670 | 1.00 | 10.00 | A | N |
| ATOM | 2894 | CA | PHE | A | 307 | −29.807 | −3.104 | 4.312 | 1.00 | 10.00 | A | C |
| ATOM | 2895 | CB | PHE | A | 307 | −31.114 | −2.322 | 4.177 | 1.00 | 10.00 | A | C |
| ATOM | 2896 | CG | PHE | A | 307 | −31.053 | −0.943 | 4.762 | 1.00 | 10.00 | A | C |
| ATOM | 2897 | CD1 | PHE | A | 307 | −31.681 | −0.656 | 5.962 | 1.00 | 10.00 | A | C |
| ATOM | 2898 | CD2 | PHE | A | 307 | −30.358 | 0.065 | 4.114 | 1.00 | 10.00 | A | C |
| ATOM | 2899 | CE1 | PHE | A | 307 | −31.616 | 0.610 | 6.508 | 1.00 | 10.00 | A | C |
| ATOM | 2900 | CE2 | PHE | A | 307 | −30.290 | 1.333 | 4.652 | 1.00 | 10.00 | A | C |
| ATOM | 2901 | CZ | PHE | A | 307 | −30.918 | 1.607 | 5.852 | 1.00 | 10.00 | A | C |
| ATOM | 2902 | C | PHE | A | 307 | −29.458 | −3.282 | 5.780 | 1.00 | 10.00 | A | C |
| ATOM | 2903 | O | PHE | A | 307 | −30.127 | −4.024 | 6.500 | 1.00 | 10.00 | A | O |
| ATOM | 2904 | N | ASP | A | 308 | −28.414 | −2.606 | 6.216 | 1.00 | 10.00 | A | N |
| ATOM | 2906 | CA | ASP | A | 308 | −27.971 | −2.689 | 7.598 | 1.00 | 10.00 | A | C |
| ATOM | 2907 | CB | ASP | A | 308 | −26.692 | −3.520 | 7.692 | 1.00 | 10.00 | A | C |
| ATOM | 2908 | CG | ASP | A | 308 | −26.500 | −4.167 | 9.046 | 1.00 | 10.00 | A | C |
| ATOM | 2909 | OD1 | ASP | A | 308 | −27.360 | −3.982 | 9.938 | 1.00 | 10.00 | A | O |
| ATOM | 2910 | OD2 | ASP | A | 308 | −25.481 | −4.868 | 9.229 | 1.00 | 10.00 | A | O |
| ATOM | 2911 | C | ASP | A | 308 | −27.752 | −1.293 | 8.166 | 1.00 | 10.00 | A | C |
| ATOM | 2912 | O | ASP | A | 308 | −26.719 | −0.661 | 7.933 | 1.00 | 10.00 | A | O |
| ATOM | 2913 | N | ASP | A | 309 | −28.743 | −0.805 | 8.889 | 1.00 | 10.00 | A | N |
| ATOM | 2915 | CA | ASP | A | 309 | −28.675 | 0.523 | 9.482 | 1.00 | 10.00 | A | C |
| ATOM | 2916 | CB | ASP | A | 309 | −30.072 | 1.145 | 9.549 | 1.00 | 10.00 | A | C |
| ATOM | 2917 | CG | ASP | A | 309 | −30.112 | 2.429 | 10.351 | 1.00 | 10.00 | A | C |
| ATOM | 2918 | OD1 | ASP | A | 309 | −30.238 | 2.353 | 11.590 | 1.00 | 10.00 | A | O |
| ATOM | 2919 | OD2 | ASP | A | 309 | −30.027 | 3.517 | 9.747 | 1.00 | 10.00 | A | O |
| ATOM | 2920 | C | ASP | A | 309 | −28.057 | 0.481 | 10.874 | 1.00 | 10.00 | A | C |
| ATOM | 2921 | O | ASP | A | 309 | −28.470 | −0.310 | 11.726 | 1.00 | 10.00 | A | O |
| ATOM | 2922 | N | TYR | A | 310 | −27.058 | 1.327 | 11.092 | 1.00 | 10.00 | A | N |
| ATOM | 2924 | CA | TYR | A | 310 | −26.394 | 1.405 | 12.386 | 1.00 | 10.00 | A | C |
| ATOM | 2925 | CB | TYR | A | 310 | −24.882 | 1.212 | 12.255 | 1.00 | 10.00 | A | C |
| ATOM | 2926 | CG | TYR | A | 310 | −24.467 | 0.006 | 11.446 | 1.00 | 10.00 | A | C |
| ATOM | 2927 | CD1 | TYR | A | 310 | −24.585 | −1.280 | 11.960 | 1.00 | 10.00 | A | C |
| ATOM | 2928 | CD2 | TYR | A | 310 | −23.945 | 0.157 | 10.170 | 1.00 | 10.00 | A | C |
| ATOM | 2929 | CE1 | TYR | A | 310 | −24.192 | −2.379 | 11.222 | 1.00 | 10.00 | A | C |
| ATOM | 2930 | CE2 | TYR | A | 310 | −23.554 | −0.936 | 9.425 | 1.00 | 10.00 | A | C |
| ATOM | 2931 | CZ | TYR | A | 310 | −23.678 | −2.198 | 9.955 | 1.00 | 10.00 | A | C |
| ATOM | 2932 | OH | TYR | A | 310 | −23.277 | −3.286 | 9.215 | 1.00 | 10.00 | A | O |
| ATOM | 2934 | C | TYR | A | 310 | −26.686 | 2.744 | 13.045 | 1.00 | 10.00 | A | C |
| ATOM | 2935 | O | TYR | A | 310 | −26.104 | 3.083 | 14.074 | 1.00 | 10.00 | A | O |
| ATOM | 2936 | N | VAL | A | 311 | −27.585 | 3.508 | 12.439 | 1.00 | 10.00 | A | N |
| ATOM | 2938 | CA | VAL | A | 311 | −27.968 | 4.807 | 12.976 | 1.00 | 10.00 | A | C |
| ATOM | 2939 | CB | VAL | A | 311 | −28.542 | 5.737 | 11.887 | 1.00 | 10.00 | A | C |
| ATOM | 2940 | CG1 | VAL | A | 311 | −28.826 | 7.120 | 12.460 | 1.00 | 10.00 | A | C |
| ATOM | 2941 | CG2 | VAL | A | 311 | −27.584 | 5.841 | 10.707 | 1.00 | 10.00 | A | C |
| ATOM | 2942 | C | VAL | A | 311 | −28.996 | 4.607 | 14.085 | 1.00 | 10.00 | A | C |
| ATOM | 2943 | O | VAL | A | 311 | −28.906 | 5.213 | 15.152 | 1.00 | 10.00 | A | O |
| ATOM | 2944 | N | ALA | A | 312 | −29.962 | 3.733 | 13.833 | 1.00 | 10.00 | A | N |
| ATOM | 2946 | CA | ALA | A | 312 | −30.991 | 3.427 | 14.813 | 1.00 | 10.00 | A | C |
| ATOM | 2947 | CB | ALA | A | 312 | −32.253 | 2.929 | 14.127 | 1.00 | 10.00 | A | C |
| ATOM | 2948 | C | ALA | A | 312 | −30.465 | 2.391 | 15.800 | 1.00 | 10.00 | A | C |
| ATOM | 2949 | O | ALA | A | 312 | −30.864 | 2.362 | 16.964 | 1.00 | 10.00 | A | O |
| ATOM | 2950 | N | ASN | A | 313 | −29.564 | 1.543 | 15.319 | 1.00 | 10.00 | A | N |
| ATOM | 2952 | CA | ASN | A | 313 | −28.952 | 0.511 | 16.152 | 1.00 | 10.00 | A | C |
| ATOM | 2953 | CB | ASN | A | 313 | −29.449 | −0.890 | 15.772 | 1.00 | 10.00 | A | C |
| ATOM | 2954 | CG | ASN | A | 313 | −28.783 | −2.001 | 16.575 | 1.00 | 10.00 | A | C |
| ATOM | 2955 | OD1 | ASN | A | 313 | −28.485 | −3.074 | 16.045 | 1.00 | 10.00 | A | O |
| ATOM | 2956 | ND2 | ASN | A | 313 | −28.563 | −1.768 | 17.863 | 1.00 | 10.00 | A | N |
| ATOM | 2959 | C | ASN | A | 313 | −27.433 | 0.578 | 16.057 | 1.00 | 10.00 | A | C |
| ATOM | 2960 | O | ASN | A | 313 | −26.829 | −0.032 | 15.178 | 1.00 | 10.00 | A | O |
| ATOM | 2961 | N | PRO | A | 314 | −26.801 | 1.347 | 16.947 | 1.00 | 10.00 | A | N |
| ATOM | 2962 | CA | PRO | A | 314 | −25.348 | 1.487 | 16.967 | 1.00 | 10.00 | A | C |
| ATOM | 2963 | CB | PRO | A | 314 | −25.112 | 2.667 | 17.911 | 1.00 | 10.00 | A | C |
| ATOM | 2964 | CG | PRO | A | 314 | −26.300 | 2.682 | 18.806 | 1.00 | 10.00 | A | C |
| ATOM | 2965 | CD | PRO | A | 314 | −27.452 | 2.153 | 17.995 | 1.00 | 10.00 | A | C |
| ATOM | 2966 | C | PRO | A | 314 | −24.662 | 0.236 | 17.505 | 1.00 | 10.00 | A | C |
| ATOM | 2967 | O | PRO | A | 314 | −25.095 | −0.352 | 18.501 | 1.00 | 10.00 | A | O |
| ATOM | 2968 | N | LYS | A | 315 | −23.593 | −0.165 | 16.836 | 1.00 | 10.00 | A | N |
| ATOM | 2970 | CA | LYS | A | 315 | −22.832 | −1.335 | 17.240 | 1.00 | 10.00 | A | C |
| ATOM | 2971 | CB | LYS | A | 315 | −21.814 | −1.704 | 16.154 | 1.00 | 10.00 | A | C |
| ATOM | 2972 | CG | LYS | A | 315 | −22.423 | −2.384 | 14.941 | 1.00 | 10.00 | A | C |
| ATOM | 2973 | CD | LYS | A | 315 | −22.853 | −3.798 | 15.272 | 1.00 | 10.00 | A | C |
| ATOM | 2974 | CE | LYS | A | 315 | −23.482 | −4.477 | 14.073 | 1.00 | 10.00 | A | C |
| ATOM | 2975 | NZ | LYS | A | 315 | −23.829 | −5.886 | 14.369 | 1.00 | 10.00 | A | N |
| ATOM | 2979 | C | LYS | A | 315 | −22.120 | −1.064 | 18.561 | 1.00 | 10.00 | A | C |
| ATOM | 2980 | O | LYS | A | 315 | −21.836 | 0.092 | 18.887 | 1.00 | 10.00 | A | O |
| ATOM | 2981 | N | PRO | A | 316 | −21.828 | −2.118 | 19.346 | 1.00 | 10.00 | A | N |
| ATOM | 2982 | CA | PRO | A | 316 | −21.132 | −1.979 | 20.636 | 1.00 | 10.00 | A | C |
| ATOM | 2983 | CB | PRO | A | 316 | −20.945 | −3.425 | 21.103 | 1.00 | 10.00 | A | C |
| ATOM | 2984 | CG | PRO | A | 316 | −22.010 | −4.190 | 20.396 | 1.00 | 10.00 | A | C |
| ATOM | 2985 | CD | PRO | A | 316 | −22.178 | −3.523 | 19.061 | 1.00 | 10.00 | A | C |
| ATOM | 2986 | C | PRO | A | 316 | −19.768 | −1.308 | 20.474 | 1.00 | 10.00 | A | C |
| ATOM | 2987 | O | PRO | A | 316 | −19.214 | −0.762 | 21.422 | 1.00 | 10.00 | A | O |
| ATOM | 2988 | N | ASN | A | 317 | −19.240 | −1.349 | 19.255 | 1.00 | 10.00 | A | N |
| ATOM | 2990 | CA | ASN | A | 317 | −17.944 | −0.752 | 18.946 | 1.00 | 10.00 | A | C |
| ATOM | 2991 | CB | ASN | A | 317 | −17.317 | −1.447 | 17.730 | 1.00 | 10.00 | A | C |
| ATOM | 2992 | CG | ASN | A | 317 | −17.412 | −2.959 | 17.784 | 1.00 | 10.00 | A | C |
| ATOM | 2993 | OD1 | ASN | A | 317 | −18.444 | −3.537 | 17.432 | 1.00 | 10.00 | A | O |
| ATOM | 2994 | ND2 | ASN | A | 317 | −16.342 | −3.611 | 18.209 | 1.00 | 10.00 | A | N |
| ATOM | 2997 | C | ASN | A | 317 | −18.083 | 0.737 | 18.651 | 1.00 | 10.00 | A | C |
| ATOM | 2998 | O | ASN | A | 317 | −17.099 | 1.412 | 18.347 | 1.00 | 10.00 | A | O |
| ATOM | 2999 | N | GLY | A | 318 | −19.306 | 1.248 | 18.747 | 1.00 | 10.00 | A | N |
| ATOM | 3001 | CA | GLY | A | 318 | −19.544 | 2.650 | 18.471 | 1.00 | 10.00 | A | C |
| ATOM | 3002 | C | GLY | A | 318 | −19.686 | 2.901 | 16.986 | 1.00 | 10.00 | A | C |
| ATOM | 3003 | O | GLY | A | 318 | −19.616 | 4.041 | 16.525 | 1.00 | 10.00 | A | O |
| ATOM | 3004 | N | TYR | A | 319 | −19.888 | 1.824 | 16.241 | 1.00 | 10.00 | A | N |
| ATOM | 3006 | CA | TYR | A | 319 | −20.039 | 1.898 | 14.798 | 1.00 | 10.00 | A | C |
| ATOM | 3007 | CB | TYR | A | 319 | −19.763 | 0.518 | 14.189 | 1.00 | 10.00 | A | C |
| ATOM | 3008 | CG | TYR | A | 319 | −19.652 | 0.478 | 12.678 | 1.00 | 10.00 | A | C |
| ATOM | 3009 | CD1 | TYR | A | 319 | −19.994 | −0.670 | 11.977 | 1.00 | 10.00 | A | C |
| ATOM | 3010 | CD2 | TYR | A | 319 | −19.209 | 1.580 | 11.952 | 1.00 | 10.00 | A | C |
| ATOM | 3011 | CE1 | TYR | A | 319 | −19.898 | −0.726 | 10.602 | 1.00 | 10.00 | A | C |
| ATOM | 3012 | CE2 | TYR | A | 319 | −19.112 | 1.532 | 10.570 | 1.00 | 10.00 | A | C |
| ATOM | 3013 | CZ | TYR | A | 319 | −19.458 | 0.374 | 9.902 | 1.00 | 10.00 | A | C |
| ATOM | 3014 | OH | TYR | A | 319 | −19.357 | 0.317 | 8.528 | 1.00 | 10.00 | A | O |
| ATOM | 3016 | C | TYR | A | 319 | −21.440 | 2.374 | 14.434 | 1.00 | 10.00 | A | C |
| ATOM | 3017 | O | TYR | A | 319 | −22.418 | 1.635 | 14.579 | 1.00 | 10.00 | A | O |
| ATOM | 3018 | N | GLN | A | 320 | −21.525 | 3.615 | 13.984 | 1.00 | 10.00 | A | N |
| ATOM | 3020 | CA | GLN | A | 320 | −22.794 | 4.212 | 13.600 | 1.00 | 10.00 | A | C |
| ATOM | 3021 | CB | GLN | A | 320 | −23.114 | 5.386 | 14.524 | 1.00 | 10.00 | A | C |
| ATOM | 3022 | CG | GLN | A | 320 | −23.018 | 5.057 | 16.003 | 1.00 | 10.00 | A | C |
| ATOM | 3023 | CD | GLN | A | 320 | −22.515 | 6.225 | 16.820 | 1.00 | 10.00 | A | C |
| ATOM | 3024 | OE1 | GLN | A | 320 | −23.299 | 7.045 | 17.305 | 1.00 | 10.00 | A | O |
| ATOM | 3025 | NE2 | GLN | A | 320 | −21.207 | 6.307 | 16.991 | 1.00 | 10.00 | A | N |
| ATOM | 3028 | C | GLN | A | 320 | −22.717 | 4.707 | 12.164 | 1.00 | 10.00 | A | C |
| ATOM | 3029 | O | GLN | A | 320 | −22.085 | 5.724 | 11.885 | 1.00 | 10.00 | A | O |
| ATOM | 3030 | N | SER | A | 321 | −23.338 | 3.977 | 11.251 | 1.00 | 10.00 | A | N |
| ATOM | 3032 | CA | SER | A | 321 | −23.328 | 4.343 | 9.840 | 1.00 | 10.00 | A | C |
| ATOM | 3033 | CB | SER | A | 321 | −22.017 | 3.885 | 9.192 | 1.00 | 10.00 | A | C |
| ATOM | 3034 | OG | SER | A | 321 | −20.890 | 4.484 | 9.810 | 1.00 | 10.00 | A | O |
| ATOM | 3036 | C | SER | A | 321 | −24.506 | 3.709 | 9.103 | 1.00 | 10.00 | A | C |
| ATOM | 3037 | O | SER | A | 321 | −25.369 | 3.077 | 9.715 | 1.00 | 10.00 | A | O |
| ATOM | 3038 | N | ILE | A | 322 | −24.541 | 3.891 | 7.793 | 1.00 | 10.00 | A | N |
| ATOM | 3040 | CA | ILE | A | 322 | −25.588 | 3.322 | 6.961 | 1.00 | 10.00 | A | C |
| ATOM | 3041 | CB | ILE | A | 322 | −26.372 | 4.417 | 6.207 | 1.00 | 10.00 | A | C |
| ATOM | 3042 | CG1 | ILE | A | 322 | −26.879 | 5.487 | 7.180 | 1.00 | 10.00 | A | C |
| ATOM | 3043 | CG2 | ILE | A | 322 | −27.535 | 3.806 | 5.438 | 1.00 | 10.00 | A | C |
| ATOM | 3044 | CD1 | ILE | A | 322 | −27.550 | 6.667 | 6.506 | 1.00 | 10.00 | A | C |
| ATOM | 3045 | C | ILE | A | 322 | −24.969 | 2.361 | 5.947 | 1.00 | 10.00 | A | C |
| ATOM | 3046 | O | ILE | A | 322 | −24.037 | 2.727 | 5.222 | 1.00 | 10.00 | A | O |
| ATOM | 3047 | N | HIS | A | 323 | −25.463 | 1.133 | 5.918 | 1.00 | 10.00 | A | N |
| ATOM | 3049 | CA | HIS | A | 323 | −24.957 | 0.130 | 4.999 | 1.00 | 10.00 | A | C |
| ATOM | 3050 | CB | HIS | A | 323 | −24.307 | −1.023 | 5.774 | 1.00 | 10.00 | A | C |
| ATOM | 3051 | CG | HIS | A | 323 | −23.812 | −2.147 | 4.910 | 1.00 | 10.00 | A | C |
| ATOM | 3052 | ND1 | HIS | A | 323 | −24.443 | −3.365 | 4.793 | 1.00 | 10.00 | A | N |
| ATOM | 3054 | CD2 | HIS | A | 323 | −22.724 | −2.216 | 4.105 | 1.00 | 10.00 | A | C |
| ATOM | 3055 | CE1 | HIS | A | 323 | −23.737 | −4.116 | 3.937 | 1.00 | 10.00 | A | C |
| ATOM | 3056 | NE2 | HIS | A | 323 | −22.684 | −3.465 | 3.489 | 1.00 | 10.00 | A | N |
| ATOM | 3057 | C | HIS | A | 323 | −26.085 | −0.394 | 4.122 | 1.00 | 10.00 | A | C |
| ATOM | 3058 | O | HIS | A | 323 | −27.154 | −0.744 | 4.620 | 1.00 | 10.00 | A | O |
| ATOM | 3059 | N | THR | A | 324 | −25.843 | −0.432 | 2.821 | 1.00 | 10.00 | A | N |
| ATOM | 3061 | CA | THR | A | 324 | −26.825 | −0.923 | 1.871 | 1.00 | 10.00 | A | C |
| ATOM | 3062 | CB | THR | A | 324 | −27.864 | 0.165 | 1.496 | 1.00 | 10.00 | A | C |
| ATOM | 3063 | OG1 | THR | A | 324 | −29.006 | −0.437 | 0.865 | 1.00 | 10.00 | A | O |
| ATOM | 3065 | CG2 | THR | A | 324 | −27.264 | 1.218 | 0.573 | 1.00 | 10.00 | A | C |
| ATOM | 3066 | C | THR | A | 324 | −26.129 | −1.474 | 0.626 | 1.00 | 10.00 | A | C |
| ATOM | 3067 | O | THR | A | 324 | −25.084 | −0.966 | 0.210 | 1.00 | 10.00 | A | O |
| ATOM | 3068 | N | VAL | A | 325 | −26.682 | −2.540 | 0.064 | 1.00 | 10.00 | A | N |
| ATOM | 3070 | CA | VAL | A | 325 | −26.114 | −3.160 | −1.125 | 1.00 | 10.00 | A | C |
| ATOM | 3071 | CB | VAL | A | 325 | −25.841 | −4.667 | −0.909 | 1.00 | 10.00 | A | C |
| ATOM | 3072 | CG1 | VAL | A | 325 | −25.175 | −5.281 | −2.135 | 1.00 | 10.00 | A | C |
| ATOM | 3073 | CG2 | VAL | A | 325 | −24.983 | −4.884 | 0.329 | 1.00 | 10.00 | A | C |
| ATOM | 3074 | C | VAL | A | 325 | −27.048 | −2.980 | −2.316 | 1.00 | 10.00 | A | C |
| ATOM | 3075 | O | VAL | A | 325 | −28.188 | −3.447 | −2.299 | 1.00 | 10.00 | A | O |
| ATOM | 3076 | N | VAL | A | 326 | −26.571 | −2.289 | −3.341 | 1.00 | 10.00 | A | N |
| ATOM | 3078 | CA | VAL | A | 326 | −27.363 | −2.058 | −4.539 | 1.00 | 10.00 | A | C |
| ATOM | 3079 | CB | VAL | A | 326 | −27.438 | −0.560 | −4.908 | 1.00 | 10.00 | A | C |
| ATOM | 3080 | CG1 | VAL | A | 326 | −28.094 | 0.238 | −3.792 | 1.00 | 10.00 | A | C |
| ATOM | 3081 | CG2 | VAL | A | 326 | −26.061 | −0.002 | −5.234 | 1.00 | 10.00 | A | C |
| ATOM | 3082 | C | VAL | A | 326 | −26.808 | −2.848 | −5.722 | 1.00 | 10.00 | A | C |
| ATOM | 3083 | O | VAL | A | 326 | −25.717 | −3.427 | −5.644 | 1.00 | 10.00 | A | O |
| ATOM | 3084 | N | LEU | A | 327 | −27.570 | −2.882 | −6.804 | 1.00 | 10.00 | A | N |
| ATOM | 3086 | CA | LEU | A | 327 | −27.164 | −3.582 | −8.012 | 1.00 | 10.00 | A | C |
| ATOM | 3087 | CB | LEU | A | 327 | −28.295 | −4.499 | −8.488 | 1.00 | 10.00 | A | C |
| ATOM | 3088 | CG | LEU | A | 327 | −27.879 | −5.700 | −9.342 | 1.00 | 10.00 | A | C |
| ATOM | 3089 | CD1 | LEU | A | 327 | −28.862 | −6.844 | −9.155 | 1.00 | 10.00 | A | C |
| ATOM | 3090 | CD2 | LEU | A | 327 | −27.795 | −5.313 | −10.811 | 1.00 | 10.00 | A | C |
| ATOM | 3091 | C | LEU | A | 327 | −26.806 | −2.569 | −9.099 | 1.00 | 10.00 | A | C |
| ATOM | 3092 | O | LEU | A | 327 | −27.616 | −1.707 | −9.448 | 1.00 | 10.00 | A | O |
| ATOM | 3093 | N | GLY | A | 328 | −25.591 | −2.668 | −9.624 | 1.00 | 10.00 | A | N |
| ATOM | 3095 | CA | GLY | A | 328 | −25.151 | −1.748 | −10.655 | 1.00 | 10.00 | A | C |
| ATOM | 3096 | C | GLY | A | 328 | −25.030 | −2.417 | −12.009 | 1.00 | 10.00 | A | C |
| ATOM | 3097 | O | GLY | A | 328 | −25.141 | −3.637 | −12.107 | 1.00 | 10.00 | A | O |
| ATOM | 3098 | N | PRO | A | 329 | −24.784 | −1.639 | −13.075 | 1.00 | 10.00 | A | N |
| ATOM | 3099 | CA | PRO | A | 329 | −24.647 | −2.175 | −14.436 | 1.00 | 10.00 | A | C |
| ATOM | 3100 | CB | PRO | A | 329 | −24.546 | −0.917 | −15.306 | 1.00 | 10.00 | A | C |
| ATOM | 3101 | CG | PRO | A | 329 | −24.038 | 0.138 | −14.386 | 1.00 | 10.00 | A | C |
| ATOM | 3102 | CD | PRO | A | 329 | −24.624 | −0.175 | −13.040 | 1.00 | 10.00 | A | C |
| ATOM | 3103 | C | PRO | A | 329 | −23.389 | −3.021 | −14.589 | 1.00 | 10.00 | A | C |
| ATOM | 3104 | O | PRO | A | 329 | −23.217 | −3.733 | −15.579 | 1.00 | 10.00 | A | O |
| ATOM | 3105 | N | GLY | A | 330 | −22.515 | −2.942 | −13.597 | 1.00 | 10.00 | A | N |
| ATOM | 3107 | CA | GLY | A | 330 | −21.285 | −3.700 | −13.623 | 1.00 | 10.00 | A | C |
| ATOM | 3108 | C | GLY | A | 330 | −21.048 | −4.418 | −12.312 | 1.00 | 10.00 | A | C |
| ATOM | 3109 | O | GLY | A | 330 | −20.072 | −4.150 | −11.612 | 1.00 | 10.00 | A | O |
| ATOM | 3110 | N | GLY | A | 331 | −21.962 | −5.306 | −11.958 | 1.00 | 10.00 | A | N |
| ATOM | 3112 | CA | GLY | A | 331 | −21.830 | −6.052 | −10.729 | 1.00 | 10.00 | A | C |
| ATOM | 3113 | C | GLY | A | 331 | −23.113 | −6.089 | −9.937 | 1.00 | 10.00 | A | C |
| ATOM | 3114 | O | GLY | A | 331 | −23.667 | −5.049 | −9.583 | 1.00 | 10.00 | A | O |
| ATOM | 3115 | N | LYS | A | 332 | −23.566 | −7.293 | −9.626 | 1.00 | 10.00 | A | N |
| ATOM | 3117 | CA | LYS | A | 332 | −24.801 | −7.488 | −8.876 | 1.00 | 10.00 | A | C |
| ATOM | 3118 | CB | LYS | A | 332 | −25.378 | −8.869 | −9.186 | 1.00 | 10.00 | A | C |
| ATOM | 3119 | CG | LYS | A | 332 | −25.842 | −9.028 | −10.628 | 1.00 | 10.00 | A | C |
| ATOM | 3120 | CD | LYS | A | 332 | −25.837 | −10.481 | −11.073 | 1.00 | 10.00 | A | C |
| ATOM | 3121 | CE | LYS | A | 332 | −26.833 | −11.312 | −10.286 | 1.00 | 10.00 | A | C |
| ATOM | 3122 | NZ | LYS | A | 332 | −26.831 | −12.738 | −10.724 | 1.00 | 10.00 | A | N |
| ATOM | 3126 | C | LYS | A | 332 | −24.581 | −7.324 | −7.371 | 1.00 | 10.00 | A | C |
| ATOM | 3127 | O | LYS | A | 332 | −25.376 | −7.796 | −6.559 | 1.00 | 10.00 | A | O |
| ATOM | 3128 | N | THR | A | 333 | −23.498 | −6.650 | −7.008 | 1.00 | 10.00 | A | N |
| ATOM | 3130 | CA | THR | A | 333 | −23.166 | −6.409 | −5.612 | 1.00 | 10.00 | A | C |
| ATOM | 3131 | CB | THR | A | 333 | −22.401 | −7.600 | −4.983 | 1.00 | 10.00 | A | C |
| ATOM | 3132 | OG1 | THR | A | 333 | −23.198 | −8.787 | −5.066 | 1.00 | 10.00 | A | O |
| ATOM | 3134 | CG2 | THR | A | 333 | −22.085 | −7.323 | −3.520 | 1.00 | 10.00 | A | C |
| ATOM | 3135 | C | THR | A | 333 | −22.340 | −5.132 | −5.470 | 1.00 | 10.00 | A | C |
| ATOM | 3136 | O | THR | A | 333 | −21.159 | −5.091 | −5.830 | 1.00 | 10.00 | A | O |
| ATOM | 3137 | N | VAL | A | 334 | −22.982 | −4.074 | −4.998 | 1.00 | 10.00 | A | N |
| ATOM | 3139 | CA | VAL | A | 334 | −22.308 | −2.802 | −4.781 | 1.00 | 10.00 | A | C |
| ATOM | 3140 | CB | VAL | A | 334 | −22.833 | −1.699 | −5.729 | 1.00 | 10.00 | A | C |
| ATOM | 3141 | CG1 | VAL | A | 334 | −22.038 | −0.413 | −5.548 | 1.00 | 10.00 | A | C |
| ATOM | 3142 | CG2 | VAL | A | 334 | −22.779 | −2.163 | −7.178 | 1.00 | 10.00 | A | C |
| ATOM | 3143 | C | VAL | A | 334 | −22.510 | −2.365 | −3.337 | 1.00 | 10.00 | A | C |
| ATOM | 3144 | O | VAL | A | 334 | −23.609 | −1.971 | −2.949 | 1.00 | 10.00 | A | O |
| ATOM | 3145 | N | GLU | A | 335 | −21.453 | −2.457 | −2.544 | 1.00 | 10.00 | A | N |
| ATOM | 3147 | CA | GLU | A | 335 | −21.518 | −2.087 | −1.136 | 1.00 | 10.00 | A | C |
| ATOM | 3148 | CB | GLU | A | 335 | −20.438 | −2.826 | −0.334 | 1.00 | 10.00 | A | C |
| ATOM | 3149 | CG | GLU | A | 335 | −20.458 | −2.526 | 1.157 | 1.00 | 10.00 | A | C |
| ATOM | 3150 | CD | GLU | A | 335 | −19.402 | −3.285 | 1.940 | 1.00 | 10.00 | A | C |
| ATOM | 3151 | OE1 | GLU | A | 335 | −18.994 | −4.382 | 1.502 | 1.00 | 10.00 | A | O |
| ATOM | 3152 | OE2 | GLU | A | 335 | −18.987 | −2.795 | 3.013 | 1.00 | 10.00 | A | O |
| ATOM | 3153 | C | GLU | A | 335 | −21.378 | −0.578 | −0.947 | 1.00 | 10.00 | A | C |
| ATOM | 3154 | O | GLU | A | 335 | −20.342 | 0.005 | −1.273 | 1.00 | 10.00 | A | O |
| ATOM | 3155 | N | ILE | A | 336 | −22.425 | 0.048 | −0.429 | 1.00 | 10.00 | A | N |
| ATOM | 3157 | CA | ILE | A | 336 | −22.412 | 1.480 | −0.179 | 1.00 | 10.00 | A | C |
| ATOM | 3158 | CB | ILE | A | 336 | −23.680 | 2.172 | −0.723 | 1.00 | 10.00 | A | C |
| ATOM | 3159 | CG1 | ILE | A | 336 | −23.844 | 1.894 | −2.218 | 1.00 | 10.00 | A | C |
| ATOM | 3160 | CG2 | ILE | A | 336 | −23.628 | 3.670 | −0.463 | 1.00 | 10.00 | A | C |
| ATOM | 3161 | CD1 | ILE | A | 336 | −25.078 | 2.525 | −2.823 | 1.00 | 10.00 | A | C |
| ATOM | 3162 | C | ILE | A | 336 | −22.292 | 1.741 | 1.316 | 1.00 | 10.00 | A | C |
| ATOM | 3163 | O | ILE | A | 336 | −23.119 | 1.280 | 2.107 | 1.00 | 10.00 | A | O |
| ATOM | 3164 | N | GLN | A | 337 | −21.250 | 2.468 | 1.690 | 1.00 | 10.00 | A | N |
| ATOM | 3166 | CA | GLN | A | 337 | −20.988 | 2.799 | 3.081 | 1.00 | 10.00 | A | C |
| ATOM | 3167 | CB | GLN | A | 337 | −19.546 | 2.427 | 3.439 | 1.00 | 10.00 | A | C |
| ATOM | 3168 | CG | GLN | A | 337 | −19.122 | 1.039 | 2.982 | 1.00 | 10.00 | A | C |
| ATOM | 3169 | CD | GLN | A | 337 | −17.716 | 0.682 | 3.422 | 1.00 | 10.00 | A | C |
| ATOM | 3170 | OE1 | GLN | A | 337 | −16.876 | 1.564 | 3.625 | 1.00 | 10.00 | A | O |
| ATOM | 3171 | NE2 | GLN | A | 337 | −17.445 | −0.611 | 3.564 | 1.00 | 10.00 | A | N |
| ATOM | 3174 | C | GLN | A | 337 | −21.219 | 4.290 | 3.341 | 1.00 | 10.00 | A | C |
| ATOM | 3175 | O | GLN | A | 337 | −20.305 | 5.107 | 3.190 | 1.00 | 10.00 | A | O |
| ATOM | 3176 | N | ILE | A | 338 | −22.437 | 4.639 | 3.737 | 1.00 | 10.00 | A | N |
| ATOM | 3178 | CA | ILE | A | 338 | −22.790 | 6.029 | 4.017 | 1.00 | 10.00 | A | C |
| ATOM | 3179 | CB | ILE | A | 338 | −24.263 | 6.314 | 3.653 | 1.00 | 10.00 | A | C |
| ATOM | 3180 | CG1 | ILE | A | 338 | −24.553 | 5.832 | 2.228 | 1.00 | 10.00 | A | C |
| ATOM | 3181 | CG2 | ILE | A | 338 | −24.579 | 7.804 | 3.786 | 1.00 | 10.00 | A | C |
| ATOM | 3182 | CD1 | ILE | A | 338 | −26.007 | 5.949 | 1.824 | 1.00 | 10.00 | A | C |
| ATOM | 3183 | C | ILE | A | 338 | −22.569 | 6.343 | 5.493 | 1.00 | 10.00 | A | C |
| ATOM | 3184 | O | ILE | A | 338 | −22.813 | 5.496 | 6.352 | 1.00 | 10.00 | A | O |
| ATOM | 3185 | N | ARG | A | 339 | −22.068 | 7.540 | 5.777 | 1.00 | 10.00 | A | N |
| ATOM | 3187 | CA | ARG | A | 339 | −21.822 | 7.971 | 7.149 | 1.00 | 10.00 | A | C |
| ATOM | 3188 | CB | ARG | A | 339 | −20.671 | 7.180 | 7.781 | 1.00 | 10.00 | A | C |
| ATOM | 3189 | CG | ARG | A | 339 | −19.424 | 7.076 | 6.914 | 1.00 | 10.00 | A | C |
| ATOM | 3190 | CD | ARG | A | 339 | −18.794 | 5.693 | 7.020 | 1.00 | 10.00 | A | C |
| ATOM | 3191 | NE | ARG | A | 339 | −19.740 | 4.638 | 6.665 | 1.00 | 10.00 | A | N |
| ATOM | 3193 | CZ | ARG | A | 339 | −19.548 | 3.344 | 6.912 | 1.00 | 10.00 | A | C |
| ATOM | 3194 | NH1 | ARG | A | 339 | −18.441 | 2.922 | 7.510 | 1.00 | 10.00 | A | N |
| ATOM | 3197 | NH2 | ARG | A | 339 | −20.472 | 2.462 | 6.563 | 1.00 | 10.00 | A | N |
| ATOM | 3200 | C | ARG | A | 339 | −21.531 | 9.467 | 7.209 | 1.00 | 10.00 | A | C |
| ATOM | 3201 | O | ARG | A | 339 | −21.214 | 10.084 | 6.191 | 1.00 | 10.00 | A | O |
| ATOM | 3202 | N | THR | A | 340 | −21.656 | 10.042 | 8.401 | 1.00 | 10.00 | A | N |
| ATOM | 3204 | CA | THR | A | 340 | −21.397 | 11.458 | 8.608 | 1.00 | 10.00 | A | C |
| ATOM | 3205 | CB | THR | A | 340 | −22.186 | 11.969 | 9.827 | 1.00 | 10.00 | A | C |
| ATOM | 3206 | OG1 | THR | A | 340 | −22.860 | 10.866 | 10.455 | 1.00 | 10.00 | A | O |
| ATOM | 3208 | CG2 | THR | A | 340 | −23.215 | 12.994 | 9.396 | 1.00 | 10.00 | A | C |
| ATOM | 3209 | C | THR | A | 340 | −19.908 | 11.693 | 8.850 | 1.00 | 10.00 | A | C |
| ATOM | 3210 | O | THR | A | 340 | −19.137 | 10.741 | 8.946 | 1.00 | 10.00 | A | O |
| ATOM | 3211 | N | LYS | A | 341 | −19.514 | 12.955 | 8.955 | 1.00 | 10.00 | A | N |
| ATOM | 3213 | CA | LYS | A | 341 | −18.115 | 13.309 | 9.186 | 1.00 | 10.00 | A | C |
| ATOM | 3214 | CB | LYS | A | 341 | −17.909 | 14.820 | 9.082 | 1.00 | 10.00 | A | C |
| ATOM | 3215 | CG | LYS | A | 341 | −18.051 | 15.377 | 7.679 | 1.00 | 10.00 | A | C |
| ATOM | 3216 | CD | LYS | A | 341 | −16.988 | 14.823 | 6.746 | 1.00 | 10.00 | A | C |
| ATOM | 3217 | CE | LYS | A | 341 | −17.017 | 15.520 | 5.396 | 1.00 | 10.00 | A | C |
| ATOM | 3218 | NZ | LYS | A | 341 | −18.405 | 15.725 | 4.908 | 1.00 | 10.00 | A | N |
| ATOM | 3222 | C | LYS | A | 341 | −17.634 | 12.815 | 10.549 | 1.00 | 10.00 | A | C |
| ATOM | 3223 | O | LYS | A | 341 | −16.573 | 12.202 | 10.662 | 1.00 | 10.00 | A | O |
| ATOM | 3224 | N | GLN | A | 342 | −18.408 | 13.090 | 11.586 | 1.00 | 10.00 | A | N |
| ATOM | 3226 | CA | GLN | A | 342 | −18.046 | 12.647 | 12.925 | 1.00 | 10.00 | A | C |
| ATOM | 3227 | CB | GLN | A | 342 | −18.956 | 13.292 | 13.972 | 1.00 | 10.00 | A | C |
| ATOM | 3228 | CG | GLN | A | 342 | −18.251 | 13.695 | 15.262 | 1.00 | 10.00 | A | C |
| ATOM | 3229 | CD | GLN | A | 342 | −17.541 | 12.540 | 15.940 | 1.00 | 10.00 | A | C |
| ATOM | 3230 | OE1 | GLN | A | 342 | −16.339 | 12.338 | 15.754 | 1.00 | 10.00 | A | O |
| ATOM | 3231 | NE2 | GLN | A | 342 | −18.281 | 11.769 | 16.719 | 1.00 | 10.00 | A | N |
| ATOM | 3234 | C | GLN | A | 342 | −18.145 | 11.130 | 13.006 | 1.00 | 10.00 | A | C |
| ATOM | 3235 | O | GLN | A | 342 | −17.292 | 10.470 | 13.591 | 1.00 | 10.00 | A | O |
| ATOM | 3236 | N | MET | A | 343 | −19.177 | 10.580 | 12.380 | 1.00 | 10.00 | A | N |
| ATOM | 3238 | CA | MET | A | 343 | −19.396 | 9.145 | 12.386 | 1.00 | 10.00 | A | C |
| ATOM | 3239 | CB | MET | A | 343 | −20.744 | 8.794 | 11.766 | 1.00 | 10.00 | A | C |
| ATOM | 3240 | CG | MET | A | 343 | −21.892 | 8.745 | 12.762 | 1.00 | 10.00 | A | C |
| ATOM | 3241 | SD | MET | A | 343 | −22.331 | 10.367 | 13.420 | 1.00 | 10.00 | A | S |
| ATOM | 3242 | CE | MET | A | 343 | −23.645 | 9.922 | 14.556 | 1.00 | 10.00 | A | C |
| ATOM | 3243 | C | MET | A | 343 | −18.269 | 8.389 | 11.695 | 1.00 | 10.00 | A | C |
| ATOM | 3244 | O | MET | A | 343 | −17.812 | 7.361 | 12.198 | 1.00 | 10.00 | A | O |
| ATOM | 3245 | N | HIS | A | 344 | −17.813 | 8.895 | 10.554 | 1.00 | 10.00 | A | N |
| ATOM | 3247 | CA | HIS | A | 344 | −16.735 | 8.239 | 9.822 | 1.00 | 10.00 | A | C |
| ATOM | 3248 | CB | HIS | A | 344 | −16.552 | 8.813 | 8.397 | 1.00 | 10.00 | A | C |
| ATOM | 3249 | CG | HIS | A | 344 | −15.429 | 9.794 | 8.199 | 1.00 | 10.00 | A | C |
| ATOM | 3250 | ND1 | HIS | A | 344 | −15.660 | 11.149 | 8.166 | 1.00 | 10.00 | A | N |
| ATOM | 3251 | CD2 | HIS | A | 344 | −14.104 | 9.565 | 8.013 | 1.00 | 10.00 | A | C |
| ATOM | 3252 | CE1 | HIS | A | 344 | −14.484 | 11.714 | 7.965 | 1.00 | 10.00 | A | C |
| ATOM | 3253 | NE2 | HIS | A | 344 | −13.515 | 10.794 | 7.867 | 1.00 | 10.00 | A | N |
| ATOM | 3255 | C | HIS | A | 344 | −15.445 | 8.219 | 10.642 | 1.00 | 10.00 | A | C |
| ATOM | 3256 | O | HIS | A | 344 | −14.756 | 7.203 | 10.704 | 1.00 | 10.00 | A | O |
| ATOM | 3257 | N | GLU | A | 345 | −15.142 | 9.334 | 11.297 | 1.00 | 10.00 | A | N |
| ATOM | 3259 | CA | GLU | A | 345 | −13.948 | 9.420 | 12.121 | 1.00 | 10.00 | A | C |
| ATOM | 3260 | CB | GLU | A | 345 | −13.658 | 10.865 | 12.522 | 1.00 | 10.00 | A | C |
| ATOM | 3261 | CG | GLU | A | 345 | −12.795 | 11.615 | 11.525 | 1.00 | 10.00 | A | C |
| ATOM | 3262 | CD | GLU | A | 345 | −12.243 | 12.907 | 12.084 | 1.00 | 10.00 | A | C |
| ATOM | 3263 | OE1 | GLU | A | 345 | −11.482 | 12.861 | 13.072 | 1.00 | 10.00 | A | O |
| ATOM | 3264 | OE2 | GLU | A | 345 | −12.553 | 13.978 | 11.523 | 1.00 | 10.00 | A | O |
| ATOM | 3265 | C | GLU | A | 345 | −14.083 | 8.548 | 13.358 | 1.00 | 10.00 | A | C |
| ATOM | 3266 | O | GLU | A | 345 | −13.140 | 7.863 | 13.746 | 1.00 | 10.00 | A | O |
| ATOM | 3267 | N | ASP | A | 346 | −15.264 | 8.559 | 13.963 | 1.00 | 10.00 | A | N |
| ATOM | 3269 | CA | ASP | A | 346 | −15.509 | 7.764 | 15.165 | 1.00 | 10.00 | A | C |
| ATOM | 3270 | CB | ASP | A | 346 | −16.840 | 8.141 | 15.819 | 1.00 | 10.00 | A | C |
| ATOM | 3271 | CG | ASP | A | 346 | −16.698 | 8.452 | 17.298 | 1.00 | 10.00 | A | C |
| ATOM | 3272 | OD1 | ASP | A | 346 | −15.809 | 7.873 | 17.961 | 1.00 | 10.00 | A | O |
| ATOM | 3273 | OD2 | ASP | A | 346 | −17.476 | 9.288 | 17.805 | 1.00 | 10.00 | A | O |
| ATOM | 3274 | C | ASP | A | 346 | −15.481 | 6.273 | 14.862 | 1.00 | 10.00 | A | C |
| ATOM | 3275 | O | ASP | A | 346 | −15.263 | 5.453 | 15.750 | 1.00 | 10.00 | A | O |
| ATOM | 3276 | N | ALA | A | 347 | −15.715 | 5.927 | 13.609 | 1.00 | 10.00 | A | N |
| ATOM | 3278 | CA | ALA | A | 347 | −15.703 | 4.534 | 13.195 | 1.00 | 10.00 | A | C |
| ATOM | 3279 | CB | ALA | A | 347 | −16.607 | 4.322 | 11.990 | 1.00 | 10.00 | A | C |
| ATOM | 3280 | C | ALA | A | 347 | −14.282 | 4.085 | 12.881 | 1.00 | 10.00 | A | C |
| ATOM | 3281 | O | ALA | A | 347 | −13.905 | 2.942 | 13.154 | 1.00 | 10.00 | A | O |
| ATOM | 3282 | N | GLU | A | 348 | −13.501 | 4.995 | 12.304 | 1.00 | 10.00 | A | N |
| ATOM | 3284 | CA | GLU | A | 348 | −12.120 | 4.708 | 11.947 | 1.00 | 10.00 | A | C |
| ATOM | 3285 | CB | GLU | A | 348 | −11.610 | 5.710 | 10.908 | 1.00 | 10.00 | A | C |
| ATOM | 3286 | CG | GLU | A | 348 | −12.267 | 5.601 | 9.544 | 1.00 | 10.00 | A | C |
| ATOM | 3287 | CD | GLU | A | 348 | −12.085 | 4.244 | 8.903 | 1.00 | 10.00 | A | C |
| ATOM | 3288 | OE1 | GLU | A | 348 | −10.947 | 3.729 | 8.900 | 1.00 | 10.00 | A | O |
| ATOM | 3289 | OE2 | GLU | A | 348 | −13.080 | 3.691 | 8.383 | 1.00 | 10.00 | A | O |
| ATOM | 3290 | C | GLU | A | 348 | −11.209 | 4.731 | 13.170 | 1.00 | 10.00 | A | C |
| ATOM | 3291 | O | GLU | A | 348 | −10.339 | 3.872 | 13.323 | 1.00 | 10.00 | A | O |
| ATOM | 3292 | N | LEU | A | 349 | −11.404 | 5.722 | 14.032 | 1.00 | 10.00 | A | N |
| ATOM | 3294 | CA | LEU | A | 349 | −10.595 | 5.851 | 15.237 | 1.00 | 10.00 | A | C |
| ATOM | 3295 | CB | LEU | A | 349 | −10.447 | 7.320 | 15.650 | 1.00 | 10.00 | A | C |
| ATOM | 3296 | CG | LEU | A | 349 | −9.396 | 8.144 | 14.898 | 1.00 | 10.00 | A | C |
| ATOM | 3297 | CD1 | LEU | A | 349 | −8.083 | 7.383 | 14.794 | 1.00 | 10.00 | A | C |
| ATOM | 3298 | CD2 | LEU | A | 349 | −9.899 | 8.559 | 13.524 | 1.00 | 10.00 | A | C |
| ATOM | 3299 | C | LEU | A | 349 | −11.163 | 5.025 | 16.386 | 1.00 | 10.00 | A | C |
| ATOM | 3300 | O | LEU | A | 349 | −10.425 | 4.340 | 17.095 | 1.00 | 10.00 | A | O |
| ATOM | 3301 | N | GLY | A | 350 | −12.475 | 5.086 | 16.564 | 1.00 | 10.00 | A | N |
| ATOM | 3303 | CA | GLY | A | 350 | −13.111 | 4.333 | 17.631 | 1.00 | 10.00 | A | C |
| ATOM | 3304 | C | GLY | A | 350 | −13.967 | 5.200 | 18.534 | 1.00 | 10.00 | A | C |
| ATOM | 3305 | O | GLY | A | 350 | −13.804 | 5.181 | 19.759 | 1.00 | 10.00 | A | O |
| ATOM | 3306 | O2B | G4P | B | 1 | −6.478 | −9.243 | −7.189 | 1.00 | 10.00 | B | O |
| ATOM | 3307 | PB | G4P | B | 1 | −6.087 | −7.709 | −6.896 | 1.00 | 10.00 | B | P |
| ATOM | 3308 | O1B | G4P | B | 1 | −6.711 | −6.887 | −8.132 | 1.00 | 10.00 | B | O |
| ATOM | 3309 | O3B | G4P | B | 1 | −6.604 | −7.231 | −5.595 | 1.00 | 10.00 | B | O |
| ATOM | 3310 | O3A | G4P | B | 1 | −4.488 | −7.601 | −7.045 | 1.00 | 10.00 | B | O |
| ATOM | 3311 | PA | G4P | B | 1 | −3.491 | −8.021 | −5.847 | 1.00 | 10.00 | B | P |
| ATOM | 3312 | O1A | G4P | B | 1 | −2.555 | −6.891 | −5.644 | 1.00 | 10.00 | B | O |
| ATOM | 3313 | O2A | G4P | B | 1 | −4.281 | −8.500 | −4.692 | 1.00 | 10.00 | B | O |
| ATOM | 3314 | O5′ | G4P | B | 1 | −2.677 | −9.259 | −6.484 | 1.00 | 10.00 | B | O |
| ATOM | 3315 | C5′ | G4P | B | 1 | −2.066 | −9.109 | −7.768 | 1.00 | 10.00 | B | C |
| ATOM | 3316 | C4′ | G4P | B | 1 | −1.030 | −10.204 | −8.029 | 1.00 | 10.00 | B | C |
| ATOM | 3317 | O4′ | G4P | B | 1 | −1.548 | −11.539 | −8.051 | 1.00 | 10.00 | B | O |
| ATOM | 3318 | C3′ | G4P | B | 1 | −0.379 | −10.042 | −9.403 | 1.00 | 10.00 | B | C |
| ATOM | 3319 | O3′ | G4P | B | 1 | 0.853 | −9.329 | −9.269 | 1.00 | 10.00 | B | O |
| ATOM | 3320 | PC | G4P | B | 1 | 1.645 | −8.845 | −10.588 | 1.00 | 10.00 | B | P |
| ATOM | 3321 | O1C | G4P | B | 1 | 0.705 | −8.853 | −11.730 | 1.00 | 10.00 | B | O |
| ATOM | 3322 | O2C | G4P | B | 1 | 2.873 | −9.662 | −10.692 | 1.00 | 10.00 | B | O |
| ATOM | 3323 | O3C | G4P | B | 1 | 2.033 | −7.326 | −10.225 | 1.00 | 10.00 | B | O |
| ATOM | 3324 | PD | G4P | B | 1 | 3.407 | −6.971 | −9.469 | 1.00 | 10.00 | B | P |
| ATOM | 3325 | O1D | G4P | B | 1 | 4.559 | −6.820 | −10.586 | 1.00 | 10.00 | B | O |
| ATOM | 3326 | O2D | G4P | B | 1 | 3.196 | −5.491 | −8.870 | 1.00 | 10.00 | B | O |
| ATOM | 3327 | O3D | G4P | B | 1 | 3.769 | −7.943 | −8.414 | 1.00 | 10.00 | B | O |
| ATOM | 3328 | C2′ | G4P | B | 1 | −0.073 | −11.481 | −9.807 | 1.00 | 10.00 | B | C |
| ATOM | 3329 | O2′ | G4P | B | 1 | 1.316 | −11.606 | −10.107 | 1.00 | 10.00 | B | O |
| ATOM | 3331 | C1′ | G4P | B | 1 | −0.387 | −12.239 | −8.517 | 1.00 | 10.00 | B | C |
| ATOM | 3332 | N9 | G4P | B | 1 | −0.616 | −13.671 | −8.814 | 1.00 | 10.00 | B | N |
| ATOM | 3333 | C8 | G4P | B | 1 | −1.772 | −14.326 | −8.730 | 1.00 | 10.00 | B | C |
| ATOM | 3334 | N7 | G4P | B | 1 | −1.590 | −15.595 | −9.095 | 1.00 | 10.00 | B | N |
| ATOM | 3335 | C5 | G4P | B | 1 | −0.308 | −15.757 | −9.404 | 1.00 | 10.00 | B | C |
| ATOM | 3336 | C4 | G4P | B | 1 | 0.320 | −14.528 | −9.226 | 1.00 | 10.00 | B | C |
| ATOM | 3337 | N3 | G4P | B | 1 | 1.631 | −14.382 | −9.461 | 1.00 | 10.00 | B | N |
| ATOM | 3338 | C2 | G4P | B | 1 | 2.379 | −15.415 | −9.882 | 1.00 | 10.00 | B | C |
| ATOM | 3339 | N2 | G4P | B | 1 | 3.675 | −15.233 | −10.111 | 1.00 | 10.00 | B | N |
| ATOM | 3342 | N1 | G4P | B | 1 | 1.800 | −16.673 | −10.081 | 1.00 | 10.00 | B | N |
| ATOM | 3344 | C6 | G4P | B | 1 | 0.434 | −16.846 | −9.840 | 1.00 | 10.00 | B | C |
| ATOM | 3345 | O6 | G4P | B | 1 | −0.102 | −17.939 | −10.011 | 1.00 | 10.00 | B | O |
| END | | | | | | | | | | | | |

1. A method for identifying compounds that modulate Rel hydrolase and/or Rel synthetase activity comprising the step of employing a three dimensional structure represented by a set of atomic coordinates presented in Table 1, 2, 3, or 4 or a subset thereof, or atomic coordinates which deviate from those in Table 1, 2, 3, or 4, or a subset thereof, by a root mean square deviation (RMSD) of residue over protein backbone atoms by no more than 3 Å, and assessing the degree of fit of a candidate compound to said three-dimensional protein structure of Rel.

2. The method according to claim 1, whereby interactions of said candidate compound with one or more amino acid residues of a region on the surface of the protein defined by amino acid residues: Arg43, Ser45, His156, Thr153, Met157, Asn150, Leu154, Lys161, Arg147, Lys143, Glu168, and Ile165 of the Rel amino acid sequence as defined in SEQ ID NO: 1 or an amino acid sequence with at least 70% sequence identity to the amino acid sequence of SEQ ID NO: 1 indicate the candidate compound is a modulator of Rel hydrolase activity, or of Rel hydrolase and synthetase activity.

3. The method according to claim 1, whereby interactions of said candidate compound with one or more amino acid residues of a region on the surface of the protein defined by amino acid residues: Asn327, Tyr329, Lys325, His333, Arg277, Arg349, Gln347, Glu345, Asp272, Arg316, Lys251, Arg249, Ala275, Arg355, Ser255, and Lys186 of the Rel amino acid sequence as defined in SEQ ID NO: 1 or an amino acid sequence with at least 70% sequence identity to the amino acid sequence of SEQ ID NO: 1 indicate the candidate compound is a modulator of Rel synthetase activity or of Rel synthetase and hydrolase activity.

4. The method according to claim 1, whereby interactions of said candidate compound with one or more amino acid residues of a region on the surface of the protein defined by amino acid residues: Lys164, Asp200, Tyr201, Arg204, Tyr211, Lys212, His219, Arg221, Arg222, Arg225 of the Rel amino acid sequence as defined in SEQ ID NO: 1 or an amino acid sequence with at least 70% sequence identity to the amino acid sequence of SEQ ID NO: 1 indicate the candidate compound is an allosteric compound or an effector of the Rel synthetase and/or hydrolase activity.

5. The method according to claim 2, further comprising determining a score of said candidate compound to modulate Rel hydrolase and/or Rel synthetase activity based on the number of interactions with said amino acid residues.

6. The method according to claim 1, further comprising comparing the conformational state of Rel before and after said candidate compound binds to Rel, wherein a change in conformational state is indicative for the candidate compound to be a bona fide modulator of Rel hydrolase and/or Rel synthetase activity, preferably wherein the conformational state of Rel before candidate compound binding is the resting conformational state characterized by the atomic coordinates of Table 1.

7. The method according to claim 1, further comprising comparing the conformational state of Rel with or without said candidate compound binding to Rel, wherein a change in conformational state is indicative for the candidate compound to be a bona fide modulator of Rel hydrolase or Rel hydrolase and synthetase activity, preferably wherein the conformational state of Rel without candidate compound binding is the (P)ppGpp bound conformational state characterized by the atomic coordinates of Table 3.

8. The method according to claim 1, further comprising comparing the conformational state of Rel with or without said candidate compound binding to Rel, wherein a change in conformational state is indicative for the candidate compound to be a bona fide modulator of Rel synthetase or Rel synthetase and hydrolase activity, preferably wherein the conformational state of Rel without candidate compound binding is the AMP-G4P bound conformational state characterized by the atomic coordinates of Table 2.

9. The method according to claim 1, further comprising comparing the conformational state of Rel with or without said candidate compound binding to the allosteric site of Rel, wherein a change in conformational state is indicative for the candidate compound to be a bona fide effector of the Rel hydrolase and/or synthetase activity, preferably wherein the conformational state of Rel without candidate compound binding is the conformational state characterized by the atomic coordinates of Table 4.

10. The method according to claim 7, wherein the candidate compound is considered a Rel hydrolase inhibitor when upon binding with one or more of said Rel amino acid residues, Rel is stabilized in an open state.

11. The method according to claim 8, wherein the candidate compound is considered a Rel synthetase inhibitor when upon binding with one or more of said Rel amino acid residues, Rel is stabilized in a closed state.

12. The method according to claim 1, further comprising testing of the ability of the candidate compounds for modulating Rel synthetase and/or Rel hydrolase activity.

13. The method according to claim 1, which is a computer-implemented method, said computer comprising an inputting device, a processor, a user interface, and an outputting device, wherein said method comprises the steps of:
a) generating a three-dimensional structure of said atomic coordinates, or said subset thereof;
b) fitting the structure of step a) with the structure of a candidate compound by computational modeling; and
c) selecting a candidate compound that possesses energetically favorable interactions with the structure of step a).

14. The method according to claim 13, wherein said fitting comprises superimposing the structure of step a) with the structure of said candidate compound.

15. The method according to claim 13, wherein said modeling comprises docking modeling.

16. The method according to claim 13, wherein said candidate compound of step c) can bind to at least 1 amino acid residue of the structure of step a) without steric interference.

17. An in vitro method for identifying a compound which modulates Rel hydrolase and/or synthetase activity, comprising the steps of:
a) providing a candidate compound;
b) providing a Rel polypeptide;
c) contacting said candidate compound with said Rel polypeptide;
d) determining the hydrolase and/or synthetase activity of Rel in the presence and absence of said candidate compound; and
e) identifying said candidate compound as a compound which modulates Rel hydrolase and/or synthetase activity if a change in activity is detected.

18. The method according to claim 17, wherein said compound is inhibiting the hydrolase and/or synthetase activity of Rel; or wherein said compound is stimulating the hydrolase and/or synthetase activity of Rel.

19. The method according to claim 17, wherein said Rel polypeptide is as defined in SEQ ID NO:1 or has at least 70% sequence identity to the amino acid sequence of as defined in SEQ ID NO: 1.

20. A modulator of Rel hydrolase and/or synthetase activity obtained by the method of claim 1, wherein the modulator is
(A) an inhibitor of the Rel hydrolase and/or synthetase activity, or
(B) an effector of the Rel hydrolase and/or synthetase activity, or a compound increasing the Rel hydrolase and/or synthetase activity, and
wherein said modulator is a compound of formula (I), or an isomer, preferably a stereo-isomer or a tautomer, a solvate, a salt, preferably a pharmaceutically acceptable salt, or a prodrug thereof, 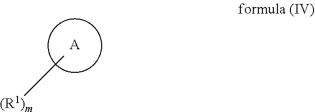
wherein m is an integer selected from 1, 2, 3, 4, 5, 6, or 7;
wherein each R¹ is independently selected from halogen, ═O, nitro, or a group comprising hydroxyl, —SH, —NH₂, C(O)OH, heterocyclyl, heteroaryl, alkyl, haloalkyl, cycloalkyl, cycloalkenyl, hydroxycarbonylalkyl, hydroxycarbonylalkenyl, alkenyl, aryl, heteroalkyl, heteroalkenyl, alkyloxy, arylalkyl, arylalkenyl-, aryl-heteroalkyl-, aryl-heteroalkenyl-, aryl-heteroaryl-; aryl-heterocyclyl-, heterocyclyl-alkyl-, heterocyclyl-alkenyl-, heterocyclyl-heteroalkyl-, heterocyclyl-heteroalkenyl-, heteroaryl-alkyl-, heteroaryl-alkenyl-, heteroaryl-heteroalkyl-, heteroaryl-heteroalkenyl-, alkyloxy-, haloalkoxy-, alkenyloxy-, aryloxy-; heteroaryloxy-, heterocylyloxy-, alkylthio-, alkenylthio-, arylthio-, heteroarylthio-, heterocyclylthio-, aryl-alkyloxy-, heteroaryl-alkyloxy-, heterocyclyl-alkyloxy-, aryl-alkylthio-, heteroaryl-alkylthio-, heterocyclyl-alkylthio-, alkyl-SO2-, alkenyl-SO2-, heteroalkyl-SO2-, heteroalkenyl-SO2-, aryl-SO2-, heteroaryl-SO2-, heterocyclyl-SO2-, Heteroaryl-heteroalkyl-heteroaryl-, Heteroaryl-heteroalkenyl-heteroaryl-, heteroaryl-alkyl-heteroaryl-, Heteroaryl-alkenyl-heteroaryl-, Aryl-heteroaryl-alkyl-, aryl-heteroaryl-heteroalkyl-, aryl-heteroaryl-alkenyl-, aryl-heteroaryl-heteroalkenyl-, Alkyloxy-aryl-alkyl-, Alkyloxy-heteroaryl-alkyl-, Alkyloxy-aryl-alkenyl-, Alkyloxy-heteroaryl-alkenyl-, Alkyloxy-heterocyclyl-alkyl-, Alkyloxy-heterocyclyl-alkenyl-, Alkyloxy-heterocyclyl-heteroalkyl-, Alkyloxy-heterocyclyl-heteroalkenyl-, aryl-alkenyl-heteroaryl-heteroalkyl, aryl-alkyl-heterocyclyl-heteroalkyl, Aryl-heteroalkyl-heteroaryl-, aryl-heteroalkenyl-heteroaryl-, aryl-heteroalkyl-heterocyclyl-, aryl-heteroaryl-heterocyclyl-, Aryl-heteroalkyl-heteroaryl-alkyl-, alkenyl-aryl-heteroalkenyl, Aryl-alkyl-heterocyclyl-SO2-, aryl-alkenyl-heterocyclyl-SO2-, heteroaryl-alkyl-heterocyclyl-SO2-, heteroaryl-alkenyl-heterocyclyl-SO2-, Aryl-heteroalkenyl-heteroaryl-alkyl-, alkenyl-aryl-heteroalkenyl-, Aryl-Alkyl-heterocyclyl-alkyl-, Aryl-Alkyl-heterocyclyl-alkenyl-, Aryl-Alkyl-heterocyclyl-heteroalkyl-, Aryl-Alkyl-heterocyclyl-heteroalkenyl-, Aryl-alkenyl-heterocyclyl-alkyloxy-, Aryl-alkyl-heterocyclyl-alkyloxy-, Aryl-alkenyl-heteroaryl-alkyloxy-, Aryl-alkyl-heteroaryl-alkyloxy-, aryl-imino-, heteroalkyl-aryl-imino-, alkenyl-aryl-imino-; and wherein each of said groups can be unsubstituted or substituted with one or more substituents each independently selected from the group comprising halogen, nitro, oxo, alkyloxy, —C(O)OH, —NH₂, hydroxycarbonylalkyl, hydroxycarbonylalkenyl, hydroxyl, alkyl, alkenyl, haloalkyl, haloalkenyl, heteroalkyl, heteroalkenyl, ═S, —SH, aryl, nitroaryl-, heteroaryl, heterocyclyl; aryl-alkyl-; aryl-alkenyl-; arylheteroalkyl-; arylheteroalkenyl-; heterocyclyl-alkyl-imino, aryl-imino-, heteroalkyl-aryl-imino-; and
wherein cycle A is selected from the group represented by formula (Ia); 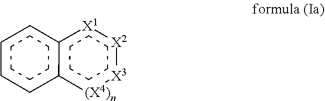
wherein the dotted line represents an optional double bond;
wherein n is an integer selected from 0 or 1;
wherein each of X¹, X², X³, and X⁴ is independently selected from the group comprising N, NH, S, O, C═O, C═S, CH, C(Z¹)₂, and N(Z²), and
wherein each Z¹ is independently selected from selected from the group comprising hydrogen, halogen, nitro, hydroxyl, —SH, —NH₂, —C(O)OH, alkyl, alkenyl, hydroxycarbonylalkyl, hydroxycarbonylalkenyl, heteroalkyl, heteroalkenyl, aryl, heteroaryl, heterocyclyl, alkyloxy, alkylthio, arylalkyl-, aryl-alkenyl-, aryl-heteroalkyl-, aryl-heteroalkenyl-, heterocyclyl-heteroalkyl, heterocyclyl-heteroalkenyl-, heteroaryl-heteroalkyl-, heteroaryl-heteroalkenyl-, Heteroaryl-heteroalkyl-heteroaryl-, Heteroaryl-heteroalkenyl-heteroaryl-, Aryl-heteroaryl-alkyl-, Aryl-heteroaryl-heteroalkyl-, Aryl-heteroaryl-alkenyl, Aryl-heteroaryl-heteroalkenyl, Alkyloxy-aryl-alkyl-, and Alkyloxy-aryl-alkenyl-; and
wherein each Z² is independently selected from the group comprising alkyl, alkenyl, hydroxycarbonylalkyl, hydroxycarbonylalkenyl, heteroalkyl, heteroalkenyl, aryl, heteroaryl, heterocyclyl, aryl-heteroalkyl-, aryl-heteroalkenyl-, heterocyclyl-heteroalkyl, heterocyclyl-heteroalkenyl-, heteroaryl-heteroalkyl-, heteroaryl-heteroalkenyl-, Heteroaryl-heteroalkyl-heteroaryl-, Heteroaryl-heteroalkenyl-heteroaryl-, Aryl-heteroaryl-alkyl-, Aryl-heteroaryl-heteroalkyl-, Aryl-heteroaryl-alkenyl, Aryl-heteroaryl-heteroalkenyl, Alkyloxy-aryl-alkyl-, and Alkyloxy-aryl-alkenyl-; or
wherein when X² and X³ are each independently selected from C(Z¹)₂ or N(Z²) as defined above, two Z¹, or Z¹ together with Z² together with the atom to which they are attached form a ring selected from the group comprising heterocyclyl, cycloalkyl, cycloalkenyl, aryl, and heteroaryl.
or
wherein said modulator is a compound of formula (II), or an isomer, preferably a stereo-isomer or a tautomer, a solvate, a salt, preferably a pharmaceutically acceptable salt, or a prodrug thereof, 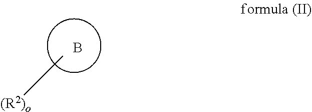
wherein o is an integer selected from 1, 2, 3, 4, 5, 6, or 7; and preferably selected from 1, 2, 3, 4, or 5, and preferably selected from 1, 2, 3, or 4;
wherein each R² is independently selected from halogen, ═S, ═O, nitro, or a group comprising hydroxyl, —SH, —NH₂, —C(O)OH, alkyl, alkenyl, haloalkyl, cycloalkyl, cycloalkenyl, heteroalkyl, heteroalkenyl, aryl, heteroaryl, heterocyclyl, arylalkyl-, arylalkenyl-, aryl-heteroalkyl-, aryl-heteroalkenyl-, aryl-heteroaryl-; aryl-heterocyclyl-, heterocyclyl-alkyl-; heterocyclyl-alkenyl-, heterocyclyl-heteroalkyl-, heterocyclyl-heteroalkenyl-, heteroaryl-alkyl-, heteroaryl-alkenyl-, heteroaryl-heteroalkyl-, heteroaryl-heteroalkenyl-, alkyloxy, haloalkoxy, alkenyloxy, aryloxy; heteroaryloxy, heterocylyloxy, alkylthio, alkenylthio, arylthio, heteroarylthio, heterocyclylthio, aryl-alkyloxy-, heteroaryl-alkyloxy-, heterocyclyl-alkyloxy-, aryl-alkylthio-, heteroaryl-alkylthio-, heterocyclyl-alkylthio-, hydroxycarbonylalkyl-, hydroxycarbonylalkenyl-, alkyl-SO2-, alkenyl-SO2-, heteroalkyl-SO2-, heteroalkenyl-SO2-, aryl-SO2-, heteroaryl-SO2-, heterocyclyl-SO2-, heteroaryl-alkyl-SO2-; heteroaryl-alkenyl-SO2-, heteroaryl-heteroalkyl-SO2-, heteroaryl-heteroalkenyl-SO2- and heteroaryl-NH—SO2-,
and wherein each of said groups can be unsubstituted or substituted with one or more substituents each independently selected from the group comprising halogen, nitro, oxo (═O), alkyloxy, —C(O)OH, —NH₂, hydroxycarbonylalkyl, hydroxycarbonylalkenyl, hydroxyl, alkyl, alkenyl, heteroalkyl, heteroalkenyl ═S, —SH, aryl, heteroaryl, heterocyclyl.
wherein cycle B is selected from the group represented by formula (IIa); 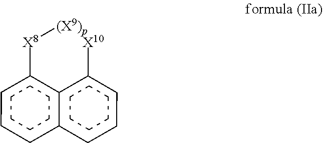
wherein the dotted line represents an optional double bond;
wherein p is an integer selected from 0 or 1;
wherein each of X⁸, X⁹, and X¹⁰ is independently selected from NH, N—OH, S, O, C═O, C═S, C(Z³)₂ and N(Z⁴), and wherein each Z³ is independently selected from the group comprising hydrogen, halogen, nitro, hydroxyl, —SH, —NH₂, —C(O)OH, alkyl, alkenyl, hydroxycarbonylalkyl, hydroxycarbonylalkenyl, heteroalkyl, heteroalkenyl, aryl, heteroaryl, heterocyclyl, and alkyloxy, and wherein each Z⁴ is independently selected from the group comprising alkyl, alkenyl, hydroxycarbonylalkyl, hydroxycarbonylalkenyl, heteroalkyl, heteroalkenyl, aryl, heteroaryl, heterocyclyl, and alkyloxy;
and preferably with the proviso that when p is 1 and X⁹ is N—OH, then X⁸ and X¹⁰ are C═O, and/or
preferably with the proviso that when p is 0 and X⁸ is NH, then X¹⁰ is C═O, or when p is 0 and X⁸ is C═O, then X¹⁰ is NH.
or
wherein said modulator is a compound of formula (III), or an isomer, preferably a stereo-isomer or a tautomer, a solvate, a salt, preferably a pharmaceutically acceptable salt, or a prodrug thereof, 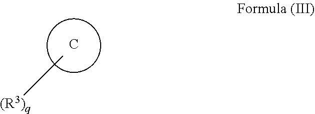
wherein q is an integer selected from 1, 2, 3, 4, 5, or 6; and
wherein each R³ is independently selected from halogen, ═S, ═O, nitro, or a group comprising hydroxyl, —SH, —NH₂, —C(O)OH, alkyl, alkenyl, haloalkyl, hydroxycarbonyl alkyl, hydroxycarbonylalkenyl, cycloalkyl, cycloalkenyl, heteroalkyl, heteroalkenyl, aryl, heteroaryl, heterocyclyl, alkyloxy, alkylthio, heterocyclyl-alkyl-, heterocyclyl-heteroalkyl-, heterocyclyl-heteroaryl, aryl-alkyl-, arylalkenyl-, aryl-heteroalkyl-, aryl-heteroalkenyl-, aryl-heteroaryl-, aryl-heterocyclyl-, heterocyclyl-alkyl-, heterocyclyl-alkenyl-, heterocyclyl-heteroalkenyl-, heteroarylalkyl-, heteroarylalkenyl-, heteroaryl-heteroalkyl-, heteroaryl-heteroalkenyl-, alkyloxy-, alkenyloxy-, aryloxy-, heteroaryloxy-, heterocylyloxy-, alkylthio-, alkenylthio-, arylthio-, heteroarylthio-, heterocyclylthio-, aryl-alkyloxy-, heteroaryl-alkyloxy-, heterocyclyl-alkyloxy-, aryl-alkylthio-, heteroaryl-alkylthio-, heterocyclyl-alkylthio-, alkyl-SO2-, alkenyl-SO2-, heteroalkyl-SO2-, heteroalkenyl-SO2-, aryl-SO2-, heteroaryl-SO2-, heterocyclyl-SO2-, heteroaryl-heteroalkyl-heteroaryl-, Heteroaryl-heteroalkenyl-heteroaryl-, heteroaryl-alkyl-heteroaryl-, Heteroaryl-alkenyl-heteroaryl-, Aryl-heteroaryl-alkyl-, aryl-heteroaryl-alkenyl-, aryl-heteroaryl-heteroalkyl-, aryl-heteroaryl-heteroalkenyl-, aryl-alkenyl-, Alkyloxy-aryl-alkyl-, Alkyloxy-heteroaryl-alkyl-, Alkyloxy-heteroaryl-alkenyl-, Alkyloxy-heterocyclyl-alkyl-, Alkyloxy-heterocyclyl-alkenyl-, Alkyloxy-heterocyclyl-heteroalkyl-, Alkyloxy-heterocyclyl-heteroalkenyl-, Aryl-heteroalkyl-heteroaryl-, aryl-heteroalkenyl-heteroaryl-, Aryl-heteroalkyl-heteroaryl-alkyl-, Aryl-heteroalkenyl-heteroaryl-alkyl-, aryl-heteroalkyl-heterocyclyl-, aryl-heteroaryl-heterocyclyl-, Aryl-alkyl-heterocyclyl Aryl-alkenyl-heterocyclyl, alkenyl-aryl-heteroalkenyl-, Aryl-Alkyl-heterocyclyl-alkyl-, Aryl-Alkyl-heterocyclyl-alkenyl-, Aryl-Alkyl-heterocyclyl-heteroalkyl-, Aryl-Alkyl-heterocyclyl-heteroalkenyl-, Aryl-alkenyl-heterocyclyl-alkyloxy-, Aryl-alkyl-heterocyclyl-alkyloxy-, Aryl-alkenyl-heteroaryl-alkyloxy-, Aryl-alkyl-heteroaryl-alkyloxy-, Aryl-alkyl-heterocyclyl-SO2-, aryl-alkenyl-heterocyclyl-SO2-, heteroaryl-alkyl-heterocyclyl-SO2-, heteroaryl-alkenyl-heterocyclyl-SO2-, aryl-amino, and aryl-NH— and wherein each of said groups can be unsubstituted or substituted with one or more substituents each independently selected from the group comprising halogen, nitro, oxo, alkyloxy, —C(O)OH, —NH₂, hydroxycarbonylalkyl, hydroxycarbonylalkenyl, hydroxyl, alkyl, alkenyl, haloalkyl, haloalkenyl, heteroalkyl, heteroalkenyl ═S, —SH, aryl, heteroaryl, heterocyclyl; aryl-alkyl-; aryl-alkenyl-; arylheteroalkyl-; arylheteroalkenyl-; and heterocyclyl-alkyl-; and
wherein cycle C is selected from the group represented by formula (IIIa); 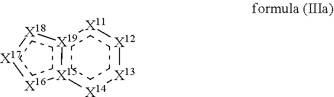
wherein the dotted line represents an optional double bond;
wherein each of X¹⁵ and X¹⁹ is independently selected from N, C, and CH,
wherein each of X¹¹, X¹², X¹³, X¹⁴, X¹⁶, X¹⁷, and X¹⁸ is independently selected from the group comprising N, NH, S, O, C═O, C═S, CH, C(Z⁵)₂ and N(Z⁶), and
wherein each Z⁵ is independently selected from the group comprising hydrogen, halogen, nitro, hydroxyl, —SH, —NH₂, —C(O)OH, alkyl, heteroalkyl, alkenyl, heteroalkenyl, haloalkyl, aryl, heteroaryl, heterocyclyl, alkyloxy, alkylthio, heterocyclyl-alkyl-, heterocyclyl-alkenyl-, heterocyclyl-heteroalkyl-, heterocyclyl-heteroalkenyl-, aryl-alkyl-, aryl-alkenyl-, aryl-heteroalkyl-; aryl-heteroalkenyl-, heteroaryl-alkyl-, heteroaryl-alkenyl-, heteroaryl-heteroalkyl-, heteroaryl-heteroalkenyl-, heteroaryl-alkyl-heteroaryl-, Heteroaryl-heteroalkyl-heteroaryl-, Aryl-heteroaryl-alkyl-, Aryl-heteroaryl-alkenyl, aryl-heteroaryl-heteroalkyl-, Aryl-heteroaryl-heteroalkenyl, aryl-heteroalkyl-heterocyclyl-, hydroxycarbonylalkyl, and hydroxycarbonylalkenyl; and
wherein each Z⁶ is independently selected from the group comprising alkyl, alkenyl, haloalkyl, hydroxycarbonylalkyl, hydroxycarbonylalkenyl, heteroalkyl, heteroalkenyl, aryl, heteroaryl, heterocyclyl, alkyloxy, arylalkyl-, arylalkenyl-, arylheteroalkyl-, arylheteroalkenyl-, heteroaryl-alkyl-, heteroaryl-alkenyl-, heterocyclyl-alkyl-, heterocyclyl-alkenyl-, heterocyclyl-heteroalkyl-; heterocyclyl-heteroalkenyl-, heteroaryl-heteroalkyl-, heteroaryl-heteroalkenyl-, heteroaryl-alkyl-heteroaryl-, Heteroaryl-heteroalkyl-heteroaryl-, Aryl-heteroaryl-alkyl-, Aryl-heteroaryl-alkenyl, aryl-heteroaryl-heteroalkyl-, and Aryl-heteroaryl-heteroalkenyl.
or
wherein said modulator is a compound of formula (IV) or an isomer, preferably a stereo-isomer or a tautomer, a solvate, a salt, preferably a pharmaceutically acceptable salt, or a prodrug thereof, 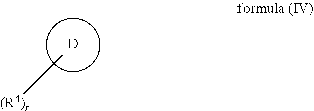
wherein cycle D is selected from the group heteroaryl, aryl, heterocyclyl, and cycloalkyl;
wherein r is an integer selected from 1, 2, 3, 4, 5 or 6; and preferably selected from 1, 2, 3 or 4; and
wherein each R⁴ is independently selected from halogen, nitro, or a group comprising hydroxyl, —NH₂, —C(O)OH, alkyl, alkenyl, haloalkyl, cycloalkyl, cycloalkenyl, heteroalkyl, heteroalkenyl, aryl, heteroaryl, heterocyclyl, arylalkyl-, arylalkenyl-, aryl-heteroalkyl-, aryl-heteroalkenyl-, aryl-heteroaryl-; aryl-heterocyclyl-, alkyl-heteroaryl-, alkenyl-heteroaryl-, heteroalkyl-heteroaryl-, heteroalkyl-heterocyclyl, heteroalkenyl-heteroaryl-, heteroalkenyl-heterocyclyl-, heterocyclyl-alkyl-; heterocyclyl-alkenyl-, heterocyclyl-heteroalkyl-, heterocyclyl-heteroalkenyl-, heterocyclyl-heterocyclyl-, heteroaryl-alkyl-, heteroaryl-alkenyl-, heteroaryl-heteroalkyl-, heteroaryl-heteroalkenyl-, alkyloxy, haloalkoxy, alkenyloxy, aryloxy; heteroaryloxy, heterocylyloxy, alkylthio, alkenylthio, arylthio, heteroarylthio, heterocyclylthio, aryl-alkyloxy-, heteroaryl-alkyloxy-, heterocyclyl-alkyloxy-, aryl-alkylthio-, heteroaryl-alkylthio-, heterocyclyl-alkylthio-, hydroxycarbonylalkyl, hydroxycarbonylalkenyl, alkyl-SO2-, alkenyl-SO2-, heteroalkyl-SO2-, heteroalkenyl-SO2-, aryl-SO2-, heteroaryl-SO2-, heterocyclyl-SO2-, heteroaryl-alkyl-SO2-; heteroaryl-alkenyl-SO2-, heteroaryl-heteroalkyl-SO2-, heteroaryl-heteroalkenyl-SO2-, heteroaryl-heteroalkyl-heteroaryl-, heteroaryl-heteroalkenyl-heteroaryl-, heteroaryl-alkyl-heteroaryl-, heteroaryl-alkenyl-heteroaryl-, Aryl-heteroaryl-alkyl-, aryl-heteroaryl-alkenyl-, aryl-heteroaryl-heteroalkyl-, aryl-heteroaryl-heteroalkenyl-, aryl-heteroalkyl-heteroaryl-; aryl-heteroalkyl-aryl-; aryl-heteroalkyl-heteroaryl-heteroalkyl-; aryl-heteroalkyl-heteroaryl-heteroalkenyl, heteroaryl-heterocylcyl-alkyl, and aryl-NH—, aryl-NH-heteroaryl-heteroalkenyl-, nitroaryl-, nitroaryl-NH—, nitroaryl-NH-heteroaryl-heteroalkenyl-; and wherein each of said groups can be unsubstituted or substituted with one or more substituents each independently selected from the group comprising halogen, nitro, oxo, alkyloxy, —C(O)OH, —NH₂, hydroxycarbonylalkyl, hydroxycarbonylalkenyl, hydroxyl, alkyl, alkenyl, heteroalkyl, heteroalkenyl ═S, —SH, aryl, nitroaryl-, nitroaryl-NH, heteroaryl, and heterocyclyl.

21-23. (canceled)

24. The modulator according to claim 20, for use in treating infections with antibiotic (multi)resistant bacteria.

25. The modulator according to claim 20, for use in treating infections with dormant, latent or persistent bacteria.

26. (canceled)

27. A computer system
(A) the computer system comprising:
a) a database containing information comprising the atomic coordinates, or a subset thereof as defined in any one of Tables 1 to 4, stored on a computer readable storage medium; and
b) an user interface to view the information; or
(B) the computer system, intended to generate three dimensional structural representations of a Rel enzyme, Rel enzyme homologues or analogues, complexes of Rel enzyme with binding compounds or modulators, or complexes of Rel enzyme homologues or analogues with binding compounds or modulators, or, to analyse or optimise binding of compounds or modulators to said Rel enzyme or homologues or analogues, or complexes thereof, the system containing computer-readable data comprising one or more of:
(a) the coordinates of the Rel enzyme structure, listed in any one of Tables 1 to 4, optionally varied by a root mean square deviation of residue backbone atoms of not more than 3 Å, or selected coordinates thereof;
(b) the coordinates of a Rel enzyme homologue or analogue generated by homology modeling of the target based on the data in (a);
(c) the coordinates of a candidate binding compound or modulator generated by interpreting X-ray crystallographic data or NMR data by reference to the coordinates of the Rel enzyme structure, listed in any one of Tables 1 to 4, optionally varied by a root mean square deviation of residue backbone atoms of not more than 3 Å, or selected coordinates thereof, and
(d) structure factor data derivable from the coordinates of (a), (b) or (c).

28. (canceled)

29. A crystal of Rel,
wherein the crystal of Rel is in its unbound resting state, comprising a structure characterized by the atomic coordinates or a subset thereof as defined in Table 1, or
wherein the crystal of Rel is in its synthetase active form, comprising a structure characterized by the atomic coordinates or a subset thereof as defined in Table 2, or
wherein the crystal of Rel is in its hydrolase active form, comprising a structure characterized by the atomic coordinates or a subset thereof as defined in Table 3, or
wherein the crystal of Rel is in its allosteric state, comprising a structure characterized by the atomic coordinates or a subset thereof as defined in Table 4.

30-32. (canceled)

33. A method for producing a medicament, pharmaceutical composition or drug, the process comprising: (a) providing a compound according to claim 20 and (b) preparing a medicament, pharmaceutical composition or drug containing said compound.

34. (canceled)

35. A computer-readable storage medium, comprising a data storage material encoded with
(A) computer readable data, wherein the data comprises one or more of
(a) the coordinates of the Rel enzyme structure, listed in any one of Tables 1 to 4, optionally varied by a root mean square deviation of residue backbone atoms of not more than 3 Å, or selected coordinates thereof;
(b) the coordinates of a Rel enzyme homologue or analogue generated by homology modeling of the target based on the data in (a);
(c) the coordinates of a candidate binding compound or modulator generated by interpreting X-ray crystallographic data or NMR data by reference to the coordinates of the Rel enzyme structure, listed in any one of Tables 1 to 4, optionally varied by a root mean square deviation of residue backbone atoms of not more than 3 Å, or selected coordinates thereof, and
(d) structure factor data derivable from the coordinates of (a), (b) or (c); or
(B) a first set of computer-readable data comprising a Fourier transform of at least a portion of the structural coordinates of the Rel enzyme listed in any one of Tables 1 to 4, optionally varied by a root mean square deviation of residue backbone atoms of not more than 3 Å, or selected coordinates thereof; which data, when combined with a second set of machine readable data comprising an X-ray diffraction pattern of a molecule or molecular complex of unknown structure, using a machine programmed with the instructions for using said first set of data and said second set of data, can determine at least a portion of the structure coordinates corresponding to the second set of machine readable data.

36. (canceled)

37. The computer system according to claim 27 further comprising a database containing information on the three dimensional structure of candidate compounds or modulators which are small molecules.

38. The computer-readable storage medium according to claim 35 further comprising a database containing information on the three dimensional structure of candidate compounds or modulators which are small molecules.

39. The method according to claim 3, further comprising determining a score of said candidate compound to modulate Rel hydrolase and/or Rel synthetase activity based on the number of interactions with said amino acid residues.

40. The method according to claim 4, further comprising determining a score of said candidate compound to modulate Rel hydrolase and/or Rel synthetase activity based on the number of interactions with said amino acid residues.
